(19) United States
(12) Patent Application Publication
(10) Pub. No.: US 2025/0374824 A1
(43) Pub. Date: Dec. 4, 2025

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: HaeYeon KIM, Gyeonggi-do (KR); Yoo-Jin DOH, Gyeonggi-do (KR); Mi-Ja LEE, Gyeonggi-do (KR); Chi-Sik KIM, Gyeonggi-do (KR); Eun-Joung CHOI, Gyeonggi-do (KR); Young-Kwang KIM, Gyeonggi-do (KR); Jeong-Eun YANG, Gyeonggi-do (KR); Hee-Ryong KANG, Gyeonggi-do (KR)

(21) Appl. No.: 19/212,061

(22) Filed: May 19, 2025

(30) Foreign Application Priority Data
May 30, 2024 (KR) 10-2024-0070682
Nov. 18, 2024 (KR) 10-2024-0163783
Apr. 25, 2025 (KR) 10-2025-0054429

(51) Int. Cl. H10K 85/60; C07D 307/77; C07D 413/10; C07D 487/04; C09K 11/02; C09K 11/06; H10K 50/11; H10K 101/00

(52) U.S. Cl. CPC H10K 85/622; C07D 307/77; C07D 413/10; C07D 487/04; C09K 11/02; C09K 11/06; H10K 85/615; H10K 85/623; H10K 85/626; H10K 85/6572; H10K 85/6574; C09K 2211/1007; C09K 2211/1011; C09K 2211/1018; H10K 50/11; H10K 2101/90

(57) ABSTRACT

The present disclosure relates to an organic electroluminescent compound and an organic electroluminescent device comprising the same. By comprising the organic electroluminescent compound according to the present disclosure, an organic electroluminescent device exhibiting lower driving voltage and/or higher efficiency and/or longer lifespan characteristics can be provided compared to a conventional organic electroluminescent device.

### TECHNICAL FIELD

The present disclosure relates to an organic electroluminescent compound and an organic electroluminescent device comprising the same.

### BACKGROUND ART

The TPD/Alq₃ bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. Therefore, an OLED having high luminous efficiency is required for long-term use and high display resolution.

Korean Patent Application Laid-Open Nos. 10-2020-0037654 and 10-2023-0006841 disclose an organic electroluminescent device having a light-emitting unit and a charge-generating layer, but do not specifically disclose an organic electroluminescent device wherein a plurality of light-emitting units having a plurality of light-emitting layers and/or a charge-generating layer comprise a compound according to the present disclosure, as in the present disclosure.

### DISCLOSURE OF THE INVENTION

### Technical Problem

The object of the present disclosure is firstly, to provide an organic electroluminescent compound that is effective in producing an organic electroluminescent device having low driving voltage and/or high efficiency and/or long lifespan characteristics, and secondly, to provide an organic electroluminescent device comprising the organic electroluminescent compound according to the present disclosure.

### Solution to Problem

As a result of intensive studies to solve the technical problem above, the present inventors found that the aforementioned objective can be achieved by an organic electroluminescent compound represented by the following Formula 1, and an organic electroluminescent device comprising the same, thereby completing the present invention.

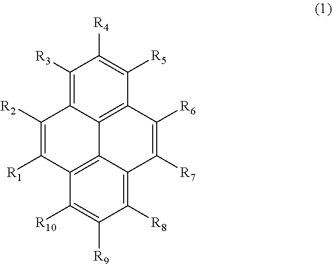

In Formula 1,

R₁ to R₁₀ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s);
provided that at least one of R₁ to R₁₀ is -L-Ar;
L represents a single bond, a substituted or unsubstituted (C₆-C₃₀)arylene, or a substituted or unsubstituted (3- to 30-membered)heteroarylene; and
Ar is represented by the following Formula 1A:

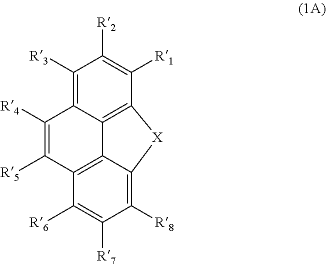

wherein in Formula 1A,
X represents —O—, —NR′₉, or —CR′₁₀R′₁₁;
any one of R′₁ to R′₈ is a site linked to the above L, and R′₁ to R′₈ which do not link to the above L each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s); and
R′₉ to R′₁₁ each independently represent a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or R′₁₀ and R′₁₁ may be linked to each other to form a ring.

### Advantageous Effects of Invention

By comprising the organic electroluminescent compound according to the present disclosure, an organic electroluminescent device having low driving voltage and/or high efficiency and/or long lifespan characteristics can be manufactured.

### EMBODIMENTS OF THE INVENTION

Hereinafter, the present disclosure will be described in detail. However, the following description is intended to explain the invention, and is not meant in any way to restrict the scope of the invention.

The present disclosure relates to an organic electroluminescent compound represented by Formula 1 and an organic electroluminescent device comprising the organic electroluminescent compound.

The present disclosure relates to an organic electroluminescent device comprising the organic electroluminescent compound represented by Formula 1 and an organic electroluminescent compound represented by Formula 2.

Herein, the term “organic electroluminescent compound” in the present disclosure means a compound that may be used in an organic electroluminescent device, and this may be comprised in any material layer constituting an organic electroluminescent device, as necessary.

Herein, the term “organic electroluminescent material” refers to a material that may be used in an organic electroluminescent device and may comprise at least one compound. Such a material may be included in any layer constituting the device, as needed. For example, the organic electroluminescent material may be a hole injection material, hole transport material, hole auxiliary material, light-emitting auxiliary material, electron-blocking material, light-emitting material (including host and dopant materials), electron buffer material, hole-blocking material, electron transport material, or electron injection material.

Herein, the “(C₁-C₃₀)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. Herein, the “(C₃-C₃₀)cycloalkyl” is meant to be a cyclic hydrocarbon substituent of saturated or partially unsaturated monocyclic or polycyclic ring having 3 to 30 ring backbone carbon atoms, in which the number of carbon atoms is preferably 3 to 20, and more preferably 3 to 7. Non-limiting examples of monocyclic cycloalkyl may include cyclopropyl, cyclobutyl, cyclopentyl, cyclohexyl, cyclohexenyl, cyclohexadienyl, cycloheptyl, cycloheptatrienyl, cyclooctyl, etc. The “(3- to 7-membered)heterocycloalkyl” in the present disclosure is meant to be a cyclic hydrocarbon substituent of a saturated or partially unsaturated monocyclic or polycyclic ring having 3 to 20 ring backbone carbon atoms, in which the number of carbon atoms is preferably 3 to 7, and more preferably 5 to 7 and including at least one heteroatom selected from the group consisting of B, N, O, S, Si, and P, preferably O, S, and N. The above heterocycloalkyl may include tetrahydrofuran, pyrrolidine, thiolan, tetrahydropyran, etc. The “(C₆-C₃₀)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15. The above aryl may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl (spirobifluorenyl), spiro[fluorene-benzofluorene]yl, azulenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, etc. The “(3- to 30-membered)heteroaryl(ene)” in the present disclosure is an aryl having 3 to 30 ring backbone atoms and including at least one heteroatom selected from the group consisting of B, N, O, S, Si, P, Se, and Ge in which the number of the ring backbone atoms is preferably 5 to 25. The number of the heteroatoms in the heteroaryl is preferably 1 to 4. The above heteroaryl may be a monocyclic ring, or a fused ring condensed with at least one benzene ring, and may be partially saturated. Also, the above heteroaryl herein may be one formed by linking at least one heteroaryl or aryl group to a heteroaryl group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified as a heteroaryl(ene) with electronic properties or a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, “a fused ring of a (C₃-C₃₀) aliphatic ring and a (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of a (C₃-C₃₀) aliphatic ring and a (C₆-C₃₀) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably this may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of ring backbone atoms is 5 to 20; according to another embodiment of the present disclosure, the number of ring backbone atoms is 5 to 15.

In addition, the term “substituted” in the expression “substituted or unsubstituted” means that a hydrogen atom in a certain functional group is replaced with another atom or functional group, i.e., a substituent. Unless otherwise specified, the substituents may not be limited to hydrogen at positions where the substituents may be substituted, and when two or more hydrogen atoms are each replaced with a substituent in a functional group, the substituents may be the same as or different from each other. The maximum number of substituents that can be substituted for a certain functional group may be the total number of valences that can be substituted for each atom forming the functional group. Preferably, the substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or dialkylamino, the substituted mono- or dialkenylamino, the substituted mono- or diarylamino, the substituted mono- or diheteroarylamino, the substituted alkylalkenylamino, the substituted alkylarylamino, the substituted alkylheteroarylamino, the substituted alkenylarylamino, the substituted alkenylheteroarylamino, the substituted arylheteroarylamino, the substituted arene ring, and the substituted heteroarene ring in the formulas of the present disclosure each independently may be substituted with least one selected from the group consisting of: deuterium; halogen; cyano; carboxyl; nitro; hydroxyl; phosphine oxide; (C₁-C₃₀)alkyl unsubstituted or substituted with deuterium; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (3- to 30-membered)heteroaryl unsubstituted or substituted with at least one of deuterium and (C₆-C₃₀)aryl; (C₆-C₃₀)aryl unsubstituted or substituted with at least one of deuterium, (C₁-C₃₀)alkyl, (C₆-C₃₀)aryl and (3- to 30-membered)heteroaryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; amino; mono- or di(C₁-C₃₀)alkylamino; mono- or di(C₂-C₃₀)alkenylamino; mono- or di(C₆-C₃₀)arylamino; mono- or di(3- to 30-membered)heteroarylamino; (C₁-C₃₀)alkyl(C₂-C₃₀)alkenylamino; (C₁-C₃₀)alkyl(C₆-C₃₀)arylamino; (C₁-C₃₀)alkyl(3- to 30-membered)heteroarylamino; (C₂-C₃₀)alkenyl(C₆-C₃₀)arylamino; (C₂-C₃₀)alkenyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; di(C₆-C₃₀)arylboronyl; (C₆-C₃₀)arylphosphinyl; di(C₁-C₃₀)alkylboronyl; (C₁-C₃₀)alkyl(C₆-C₃₀)arylboronyl; (C₆-C₃₀)ar(C₁-C₃₀)alkyl; and (C₁-C₃₀)alkyl(C₆-C₃₀)aryl; for example, the substituent may be a substituted or unsubstituted methyl, a substituted or unsubstituted phenyl, a substituted or unsubstituted naphthyl, etc.

If substituents are not indicated in the chemical formula or compound structure in the present specification, it may mean that all positions where substituents could be located are occupied by hydrogen or deuterium. That is, in the case of deuterium, which is an isotope of hydrogen, some hydrogen atoms may be replaced by deuterium, and the deuterium content may range from 0% to 100%. In cases where substituents are not indicated in the chemical formula or compound structure in the present specification, and where the deuterium content is 0%, the hydrogen content is 100%, and substituents such as hydrogen are not explicitly excluded, hydrogen and deuterium may be used interchangeably within the compound. The above-mentioned deuterium is an isotope of hydrogen, consisting of one proton and one neutron, and has a nucleus called a deuteron. It can be represented as hydrogen-2 and its elemental symbol can be written as D or ²H. The isotope, which has the same atomic number (Z) but a different mass number (A), refers to atoms with the same number of protons but a different number of neutrons.

Herein, “combinations thereof” refers to the formation of a known or chemically stable arrangement that can be conceived by a person skilled in the art through the combination of one or more components from the applicable list. For example, alkyl and deuterium may be combined to form a partially or fully deuterated alkyl group; halogen and alkyl may be combined to form a halogenated alkyl substituent; and halogen, alkyl, and aryl may be combined to form a halogenated arylalkyl. For instance, a preferred combination of substituents may include up to 50 non-hydrogen and non-deuterium atoms, or up to 40 non-hydrogen and non-deuterium atoms, or up to 30 non-hydrogen and non-deuterium atoms, and in many cases, a preferred combination of substituents may include up to 20 non-hydrogen and non-deuterium atoms.

In the formulas of the present disclosure, when multiple substituents are represented by the same symbol, each substituent represented by the same symbol may be the same or different.

Hereinafter, the organic electroluminescent compound according to one embodiment will be described in detail.

The organic electroluminescent compound according to one embodiment of the present disclosure is represented by the following Formula 1.

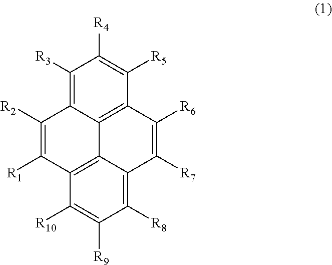

In Formula 1,
R₁ to R₁₀ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s);
provided that at least one of R₁ to R₁₀ is -L-Ar;
L represents a single bond, a substituted or unsubstituted (C₆-C₃₀)arylene, or a substituted or unsubstituted (3- to 30-membered)heteroarylene; and
Ar is represents by the following Formula 1A:

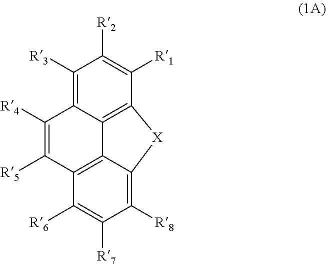

wherein in Formula 1A,
X represents —O—, —NR′₉, or —CR′₁₀R′₁₁;
any one of R′₁ to R′₈ is a site linked to the above L, and R′₁ to R′₈ which do not link to the above L each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s); and
R′₉ to R′₁₁ each independently represent a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or R′₁₀ and R′₁₁ may be linked to each other to form a ring.

According to one embodiment, the organic electroluminescent compound represented by Formula 1 may comprise at least one deuterium.

In one embodiment, R₁ to R₁₀ each independently may be hydrogen, deuterium, halogen, cyano, a substituted or unsubstituted (C₆-C₃₀)aryl, a substituted or unsubstituted (5- to 30-membered)heteroaryl, or a substituted or unsubstituted fused ring of a (C₃-C₃₀) aliphatic ring and a (C₆-C₃₀) aromatic ring, preferably, hydrogen, deuterium, or (C₆-C₂₅)aryl unsubstituted or substituted with (C₆-C₃₀)aryl, (5- to 25-membered)heteroaryl unsubstituted or substituted with (C₆-C₃₀)aryl, or a substituted or unsubstituted fused ring of (C₅-C₂₅) aliphatic ring and (C₆-C₂₅) aromatic ring, more preferably hydrogen, deuterium, or (C₆-C₁₈)aryl unsubstituted or substituted with (C₆-C₃₀)aryl, (5- to 18-membered)heteroaryl unsubstituted or substituted with (C₆-C₃₀)aryl, or a substituted or unsubstituted fused ring of a (C₅-C₁₈) aliphatic ring and a (C₆-C₁₈) aromatic ring. For example, R₁ to R₁₀ each independently may be hydrogen, deuterium, phenyl unsubstituted or substituted with tert-butyl or naphthyl, a substituted or unsubstituted p-biphenyl, a substituted or unsubstituted m-biphenyl, a substituted or unsubstituted o-biphenyl, naphthyl unsubstituted or substituted with phenyl, a substituted or unsubstituted p-terphenyl, a substituted or unsubstituted m-terphenyl, a substituted or unsubstituted o-terphenyl, a substituted or unsubstituted pyrenyl, a substituted or unsubstituted phenanthrenyl, 1,1,4,4-tetramethyltetralinyl, benzoxazolyl unsubstituted or substituted with phenyl, or a substituted or unsubstituted indolo[3,2,1-jk]carbazolyl.

In one embodiment, R₃ may be -L-Ar, preferably R₃ may be -L-Ar, and R₈ may be deuterium, halogen, cyano, a substituted or unsubstituted (C₁-C₃₀)alkyl, a substituted or unsubstituted (C₆-C₃₀)aryl, or a substituted or unsubstituted (3- to 30-membered)heteroaryl, more preferably R₃ may be -L-Ar, and R₈ may be a substituted or unsubstituted (C₆-C₃₀)aryl or a substituted or unsubstituted (3- to 30-membered)heteroaryl.

In one embodiment, L may be a single bond or a substituted or unsubstituted (C₆-C₃₀)arylene, preferably a single bond or (C₆-C₂₅)arylene unsubstituted or substituted with (C₆-C₃₀)aryl, and more preferably a single bond or (C₆-C₁₈)arylene unsubstituted or substituted with (C₆-C₃₀)aryl. For example, L may be a single bond; phenyl unsubstituted or substituted with tert-butyl; naphthyl; or phenylene unsubstituted or substituted with 1,1,4,4-tetramethyltetralinyl.

In one embodiment, X may be —O—.

In one embodiment, any one of R′₁ to R′₈ may be a site linked to the above L, and R′₁ to R′₈ which do not link to the above L each independently may be hydrogen, deuterium, or a substituted or unsubstituted (C₆-C₃₀)aryl; or may be linked to the adjacent substituents to form a ring(s), preferably hydrogen or a substituted or unsubstituted (C₆-C₂₅)aryl; or may be linked to the adjacent substituents to form a substituted or unsubstituted (3- to 30-membered) monocyclic or polycyclic, alicyclic, or aromatic ring or a combination thereof, more preferably hydrogen or a substituted or unsubstituted (C₆-C₁₈)aryl; or may be linked to the adjacent substituents to form a substituted or unsubstituted (3- to 30-membered) monocyclic or polycyclic, aromatic ring. For example, R′₁ to R′₈ which do not link to the above L each independently may be hydrogen, deuterium, a substituted or unsubstituted phenyl, or a substituted or unsubstituted naphthyl, or may be linked to the adjacent substituents to form benzene ring.

The organic electroluminescent compound represented by Formula 1 according to one embodiment can be represented by the following Formula 1-1.

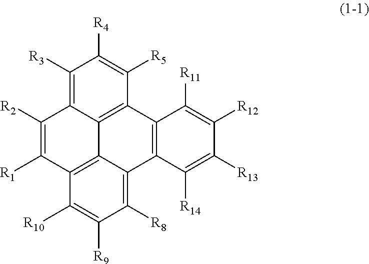

In Formula 1-1,
R₁ to R₄ and R₈ to R₁₀ are as defined in Formula 1; and
R₁₁ to R₁₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s).

According to one embodiment, the organic electroluminescent compound represented by Formula 1 may be more specifically illustrated by the following compounds, but is not limited thereto:

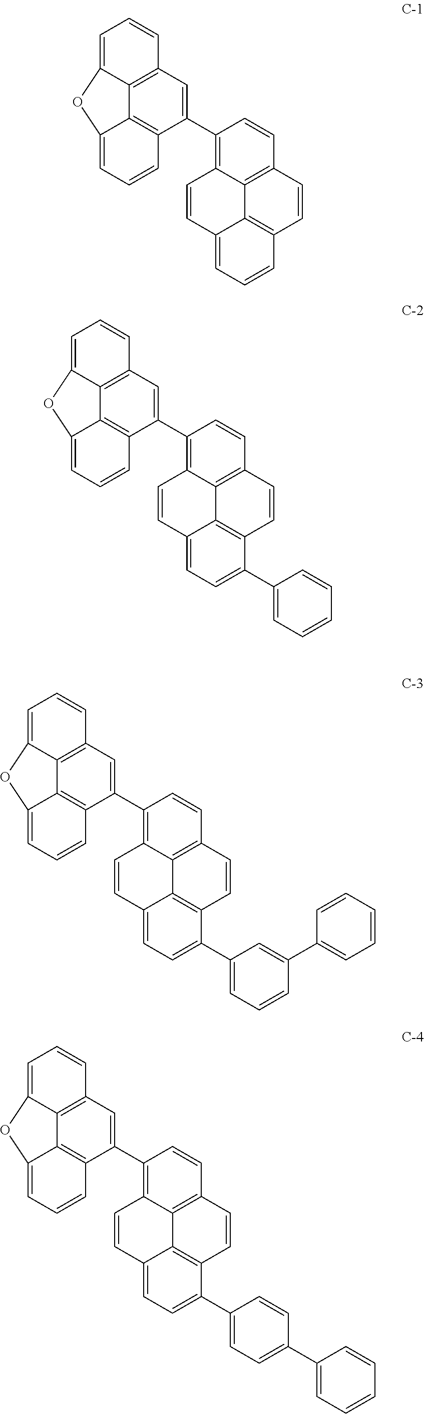 [SMILES: c1cc2ccc3ccc(-c4cc5cccc6oc7cccc4c7c56)c4ccc(c1)c2c34 ; c1ccc(-c2ccc3ccc4c(-c5cc6cccc7oc8cccc5c8c67)ccc5ccc2c3c54)cc1 ; c1ccc(-c2cccc(-c3ccc4ccc5c(-c6cc7cccc8oc9cccc6c9c78)ccc6ccc3c4c65)c2)cc1 ; c1ccc(-c2ccc(-c3ccc4ccc5c(-c6cc7cccc8oc9cccc6c9c78)ccc6ccc3c4c65)cc2)cc1]

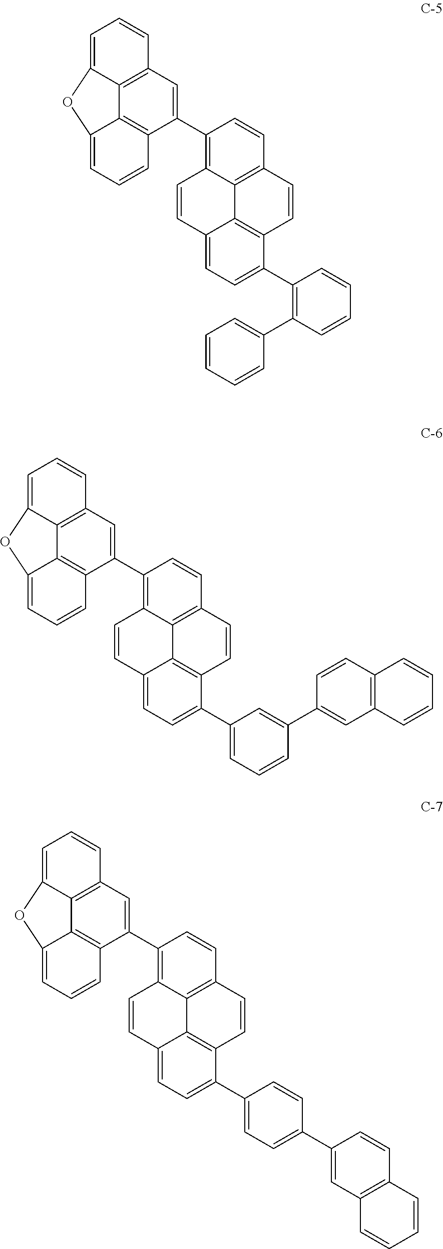 [SMILES: c1ccc(-c2ccccc2-c2ccc3ccc4c(-c5cc6cccc7oc8cccc5c8c67)ccc5ccc2c3c54)cc1 ; c1cc(-c2ccc3ccccc3c2)cc(-c2ccc3ccc4c(-c5cc6cccc7oc8cccc5c8c67)ccc5ccc2c3c54)c1 ; c1ccc2cc(-c3ccc(-c4ccc5ccc6c(-c7cc8cccc9oc%10cccc7c%10c89)ccc7ccc4c5c76)cc3)ccc2c1]

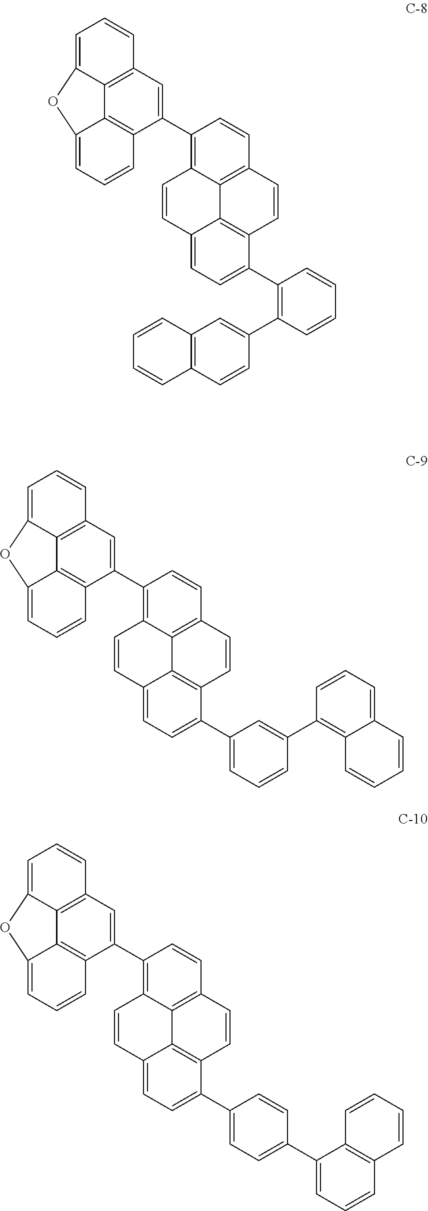 [SMILES: c1ccc(-c2ccc3ccc4c(-c5cc6cccc7oc8cccc5c8c67)ccc5ccc2c3c54)c(-c2ccc3ccccc3c2)c1 ; c1cc(-c2cccc3ccccc23)cc(-c2ccc3ccc4c(-c5cc6cccc7oc8cccc5c8c67)ccc5ccc2c3c54)c1 ; c1ccc2c(-c3ccc(-c4ccc5ccc6c(-c7cc8cccc9oc%10cccc7c%10c89)ccc7ccc4c5c76)cc3)cccc2c1]

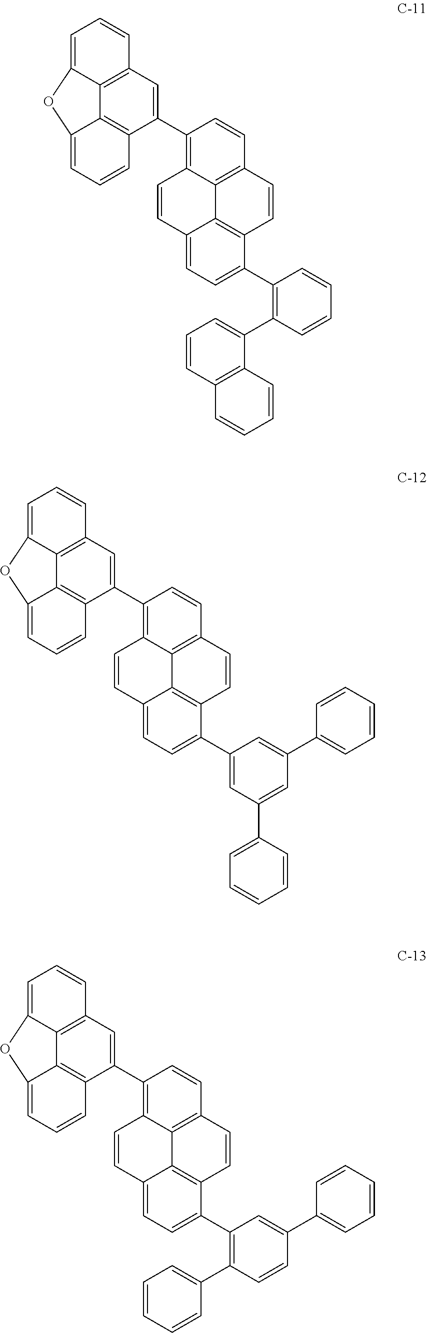 [SMILES: c1ccc(-c2ccc3ccc4c(-c5cc6cccc7oc8cccc5c8c67)ccc5ccc2c3c54)c(-c2cccc3ccccc23)c1 ; c1ccc(-c2cc(-c3ccccc3)cc(-c3ccc4ccc5c(-c6cc7cccc8oc9cccc6c9c78)ccc6ccc3c4c65)c2)cc1 ; c1ccc(-c2ccc(-c3ccccc3)c(-c3ccc4ccc5c(-c6cc7cccc8oc9cccc6c9c78)ccc6ccc3c4c65)c2)cc1]

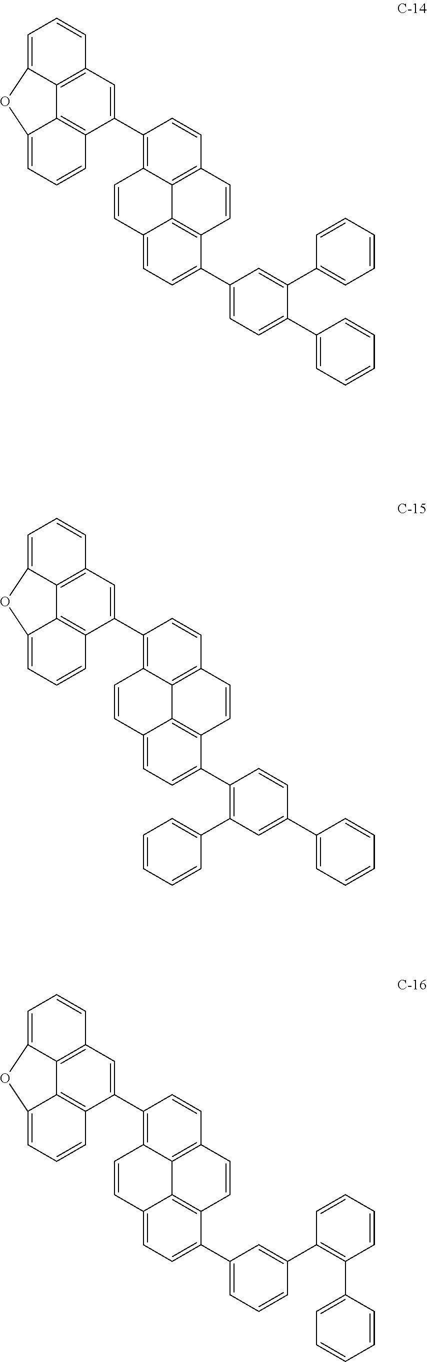 [SMILES: c1ccc(-c2ccc(-c3ccc4ccc5c(-c6cc7cccc8oc9cccc6c9c78)ccc6ccc3c4c65)cc2-c2ccccc2)cc1 ; c1ccc(-c2ccc(-c3ccc4ccc5c(-c6cc7cccc8oc9cccc6c9c78)ccc6ccc3c4c65)c(-c3ccccc3)c2)cc1 ; c1ccc(-c2ccccc2-c2cccc(-c3ccc4ccc5c(-c6cc7cccc8oc9cccc6c9c78)ccc6ccc3c4c65)c2)cc1]

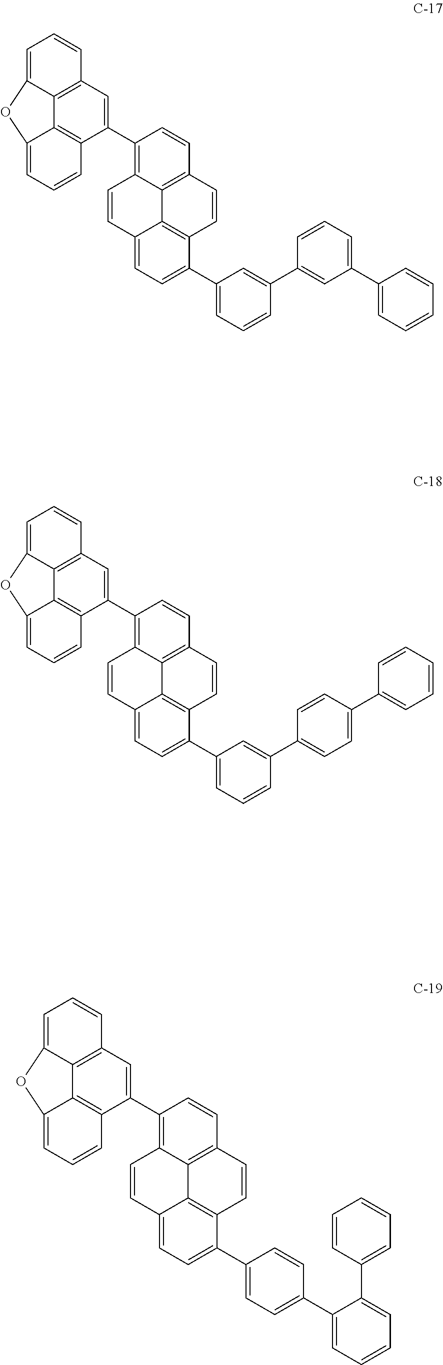 [SMILES: c1ccc(-c2cccc(-c3cccc(-c4ccc5ccc6c(-c7cc8cccc9oc%10cccc7c%10c89)ccc7ccc4c5c76)c3)c2)cc1 ; c1ccc(-c2ccc(-c3cccc(-c4ccc5ccc6c(-c7cc8cccc9oc%10cccc7c%10c89)ccc7ccc4c5c76)c3)cc2)cc1 ; c1ccc(-c2ccccc2-c2ccc(-c3ccc4ccc5c(-c6cc7cccc8oc9cccc6c9c78)ccc6ccc3c4c65)cc2)cc1]

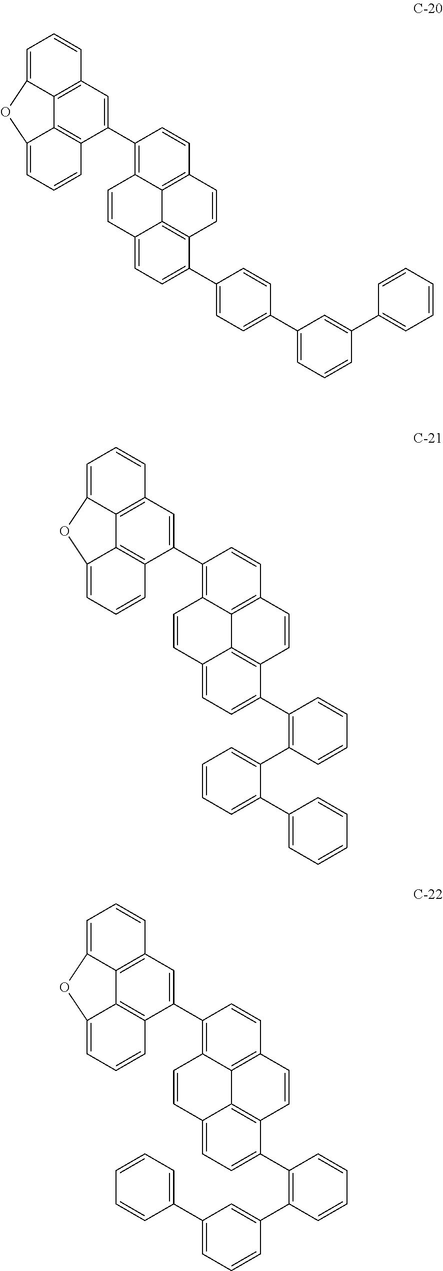 [SMILES: c1ccc(-c2cccc(-c3ccc(-c4ccc5ccc6c(-c7cc8cccc9oc%10cccc7c%10c89)ccc7ccc4c5c76)cc3)c2)cc1 ; c1ccc(-c2ccccc2-c2ccccc2-c2ccc3ccc4c(-c5cc6cccc7oc8cccc5c8c67)ccc5ccc2c3c54)cc1 ; c1ccc(-c2cccc(-c3ccccc3-c3ccc4ccc5c(-c6cc7cccc8oc9cccc6c9c78)ccc6ccc3c4c65)c2)cc1]

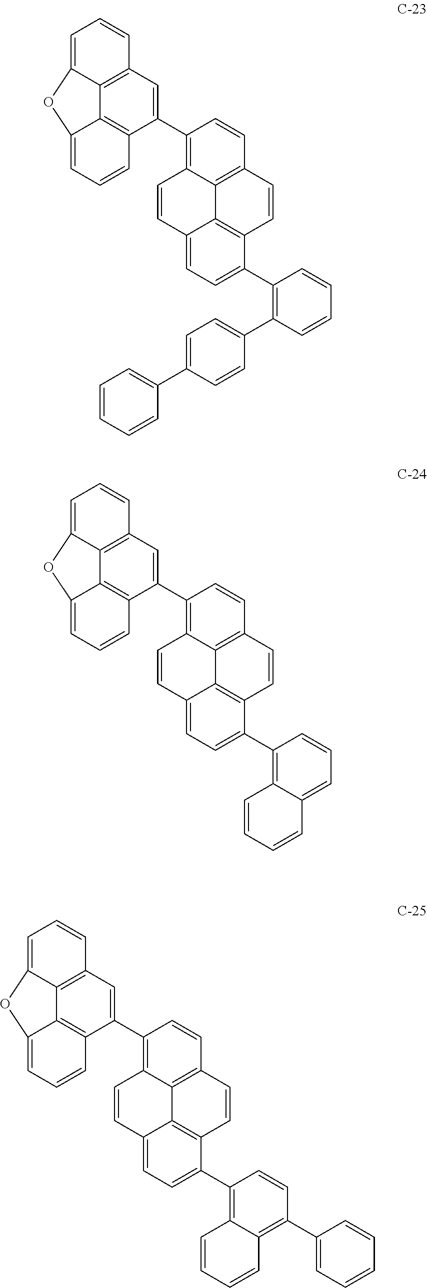 [SMILES: c1ccc(-c2ccc(-c3ccccc3-c3ccc4ccc5c(-c6cc7cccc8oc9cccc6c9c78)ccc6ccc3c4c65)cc2)cc1 ; c1ccc2c(-c3ccc4ccc5c(-c6cc7cccc8oc9cccc6c9c78)ccc6ccc3c4c65)cccc2c1 ; c1ccc(-c2ccc(-c3ccc4ccc5c(-c6cc7cccc8oc9cccc6c9c78)ccc6ccc3c4c65)c3ccccc23)cc1]

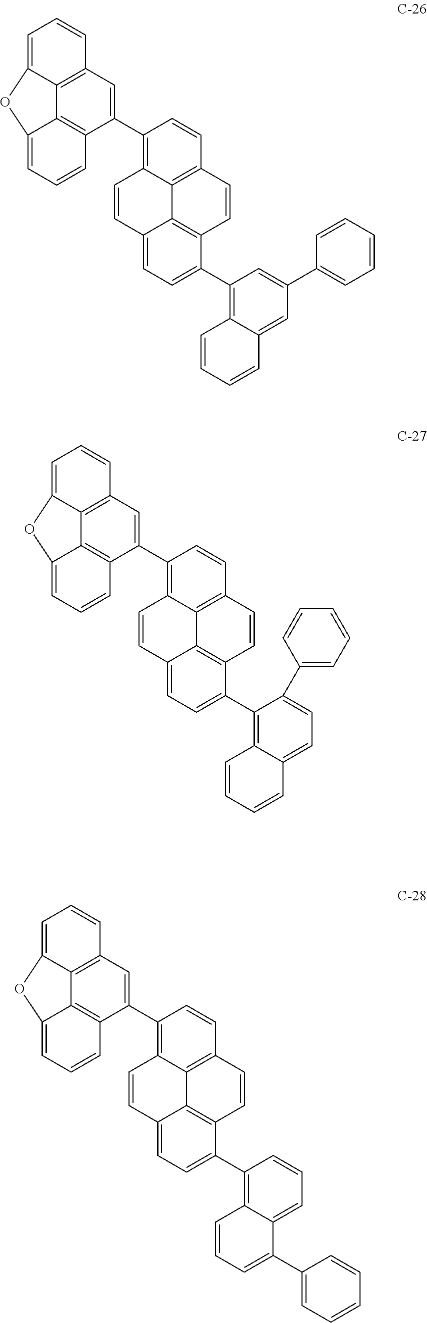 [SMILES: c1ccc(-c2cc(-c3ccc4ccc5c(-c6cc7cccc8oc9cccc6c9c78)ccc6ccc3c4c65)c3ccccc3c2)cc1 ; c1ccc(-c2ccc3ccccc3c2-c2ccc3ccc4c(-c5cc6cccc7oc8cccc5c8c67)ccc5ccc2c3c54)cc1 ; c1ccc(-c2cccc3c(-c4ccc5ccc6c(-c7cc8cccc9oc%10cccc7c%10c89)ccc7ccc4c5c76)cccc23)cc1]

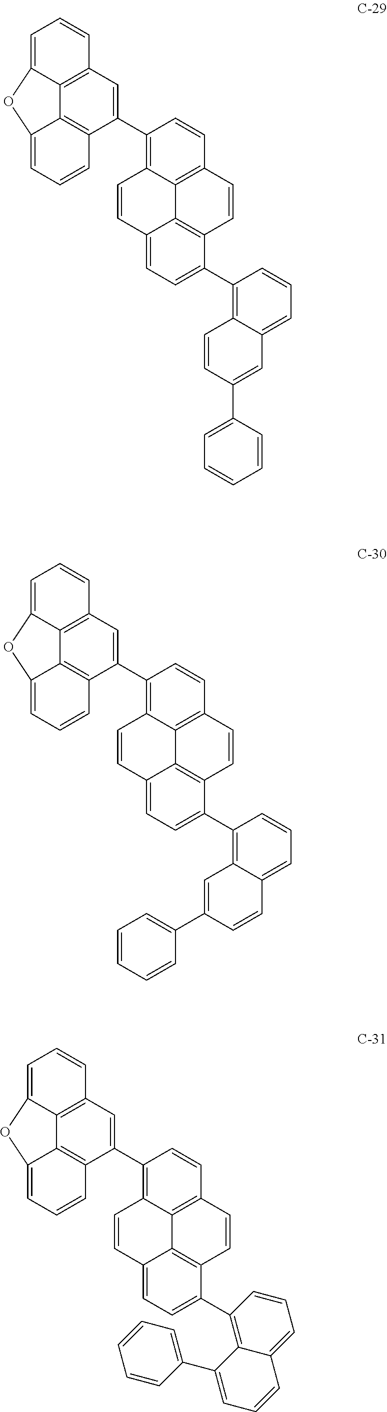 [SMILES: c1ccc(-c2ccc3c(-c4ccc5ccc6c(-c7cc8cccc9oc%10cccc7c%10c89)ccc7ccc4c5c76)cccc3c2)cc1 ; c1ccc(-c2ccc3cccc(-c4ccc5ccc6c(-c7cc8cccc9oc%10cccc7c%10c89)ccc7ccc4c5c76)c3c2)cc1 ; c1ccc(-c2cccc3cccc(-c4ccc5ccc6c(-c7cc8cccc9oc%10cccc7c%10c89)ccc7ccc4c5c76)c23)cc1]

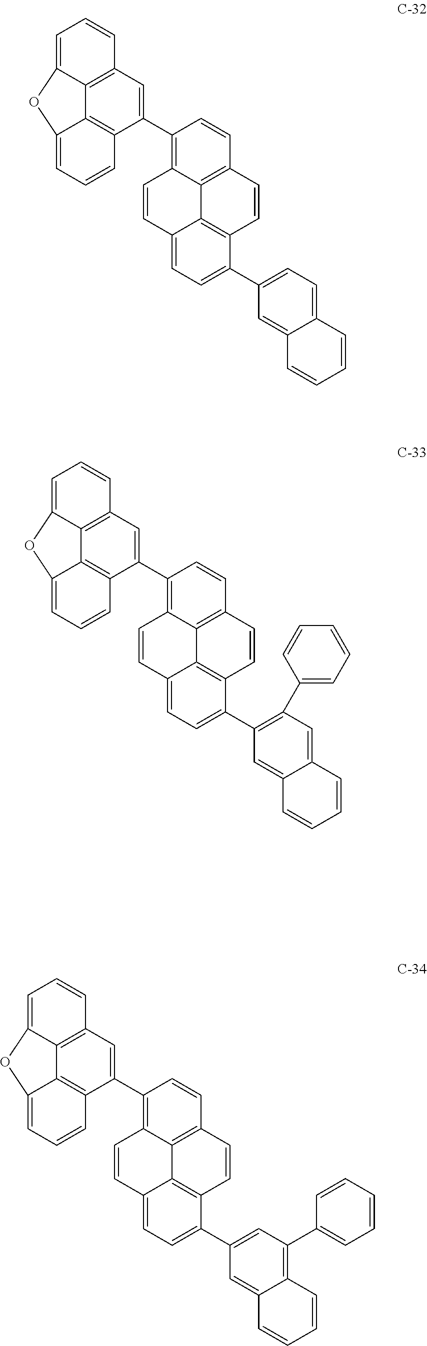 [SMILES: c1ccc2cc(-c3ccc4ccc5c(-c6cc7cccc8oc9cccc6c9c78)ccc6ccc3c4c65)ccc2c1 ; c1ccc(-c2cc3ccccc3cc2-c2ccc3ccc4c(-c5cc6cccc7oc8cccc5c8c67)ccc5ccc2c3c54)cc1 ; c1ccc(-c2cc(-c3ccc4ccc5c(-c6cc7cccc8oc9cccc6c9c78)ccc6ccc3c4c65)cc3ccccc23)cc1]

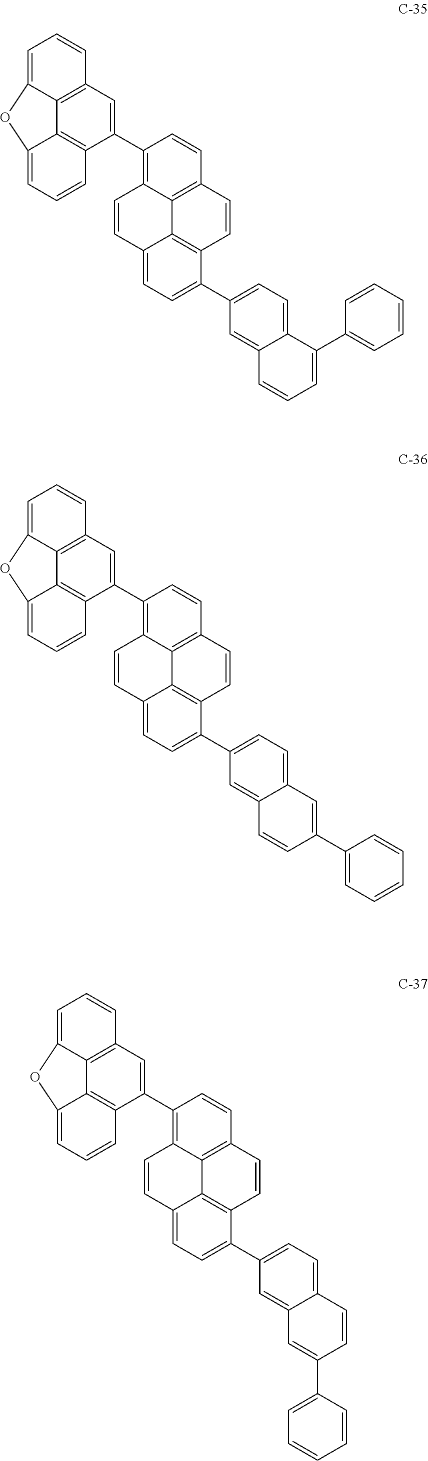 [SMILES: c1ccc(-c2cccc3cc(-c4ccc5ccc6c(-c7cc8cccc9oc%10cccc7c%10c89)ccc7ccc4c5c76)ccc23)cc1 ; c1ccc(-c2ccc3cc(-c4ccc5ccc6c(-c7cc8cccc9oc%10cccc7c%10c89)ccc7ccc4c5c76)ccc3c2)cc1 ; c1ccc(-c2ccc3ccc(-c4ccc5ccc6c(-c7cc8cccc9oc%10cccc7c%10c89)ccc7ccc4c5c76)cc3c2)cc1]

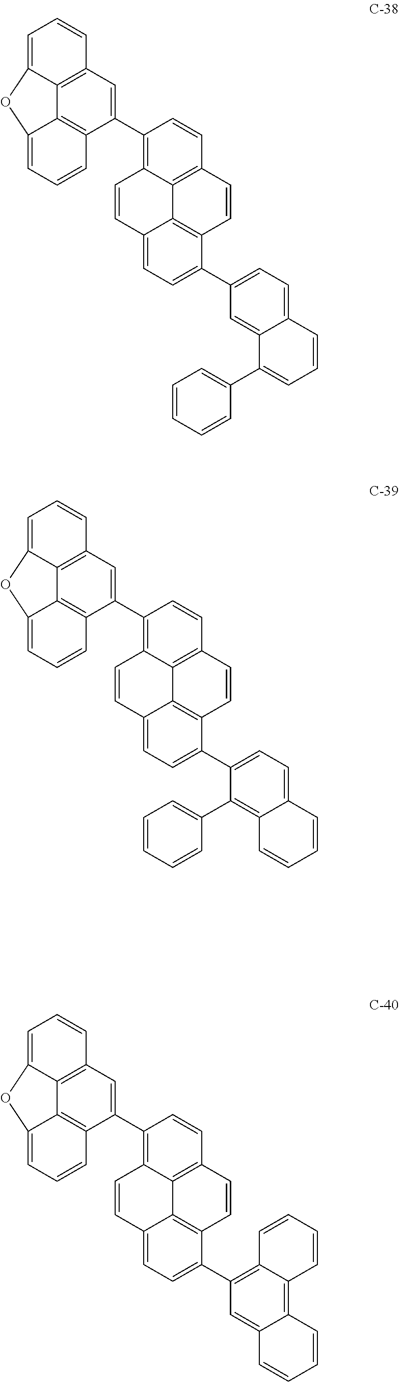 [SMILES: c1ccc(-c2cccc3ccc(-c4ccc5ccc6c(-c7cc8cccc9oc%10cccc7c%10c89)ccc7ccc4c5c76)cc23)cc1 ; c1ccc(-c2c(-c3ccc4ccc5c(-c6cc7cccc8oc9cccc6c9c78)ccc6ccc3c4c65)ccc3ccccc23)cc1 ; c1ccc2c(c1)cc(-c1ccc3ccc4c(-c5cc6cccc7oc8cccc5c8c67)ccc5ccc1c3c54)c1ccccc12]

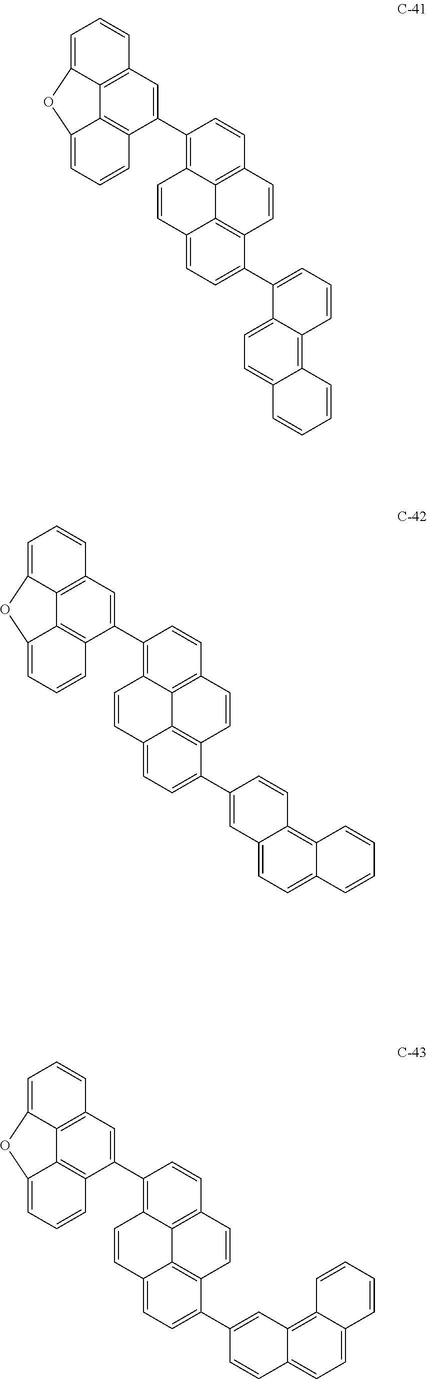 [SMILES: c1ccc2c(c1)ccc1c(-c3ccc4ccc5c(-c6cc7cccc8oc9cccc6c9c78)ccc6ccc3c4c65)cccc12 ; c1ccc2c(c1)ccc1cc(-c3ccc4ccc5c(-c6cc7cccc8oc9cccc6c9c78)ccc6ccc3c4c65)ccc12 ; c1ccc2c(c1)ccc1ccc(-c3ccc4ccc5c(-c6cc7cccc8oc9cccc6c9c78)ccc6ccc3c4c65)cc12]

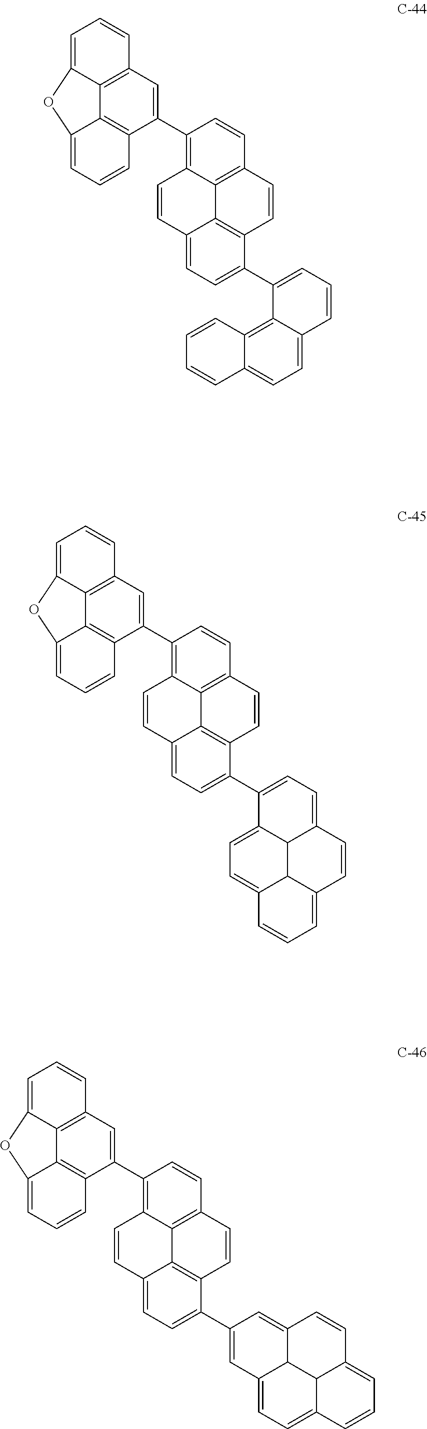 [SMILES: c1ccc2c(c1)ccc1cccc(-c3ccc4ccc5c(-c6cc7cccc8oc9cccc6c9c78)ccc6ccc3c4c65)c12 ; C1=CC2=CC=C3C(c4ccc5ccc6c(-c7cc8cccc9oc%10cccc7c%10c89)ccc7ccc4c5c76)=CC=C4C=CC(=C1)C2C43 ; C1=CC2=CC=C3C=C(c4ccc5ccc6c(-c7cc8cccc9oc%10cccc7c%10c89)ccc7ccc4c5c76)C=C4C=CC(=C1)C2C34]

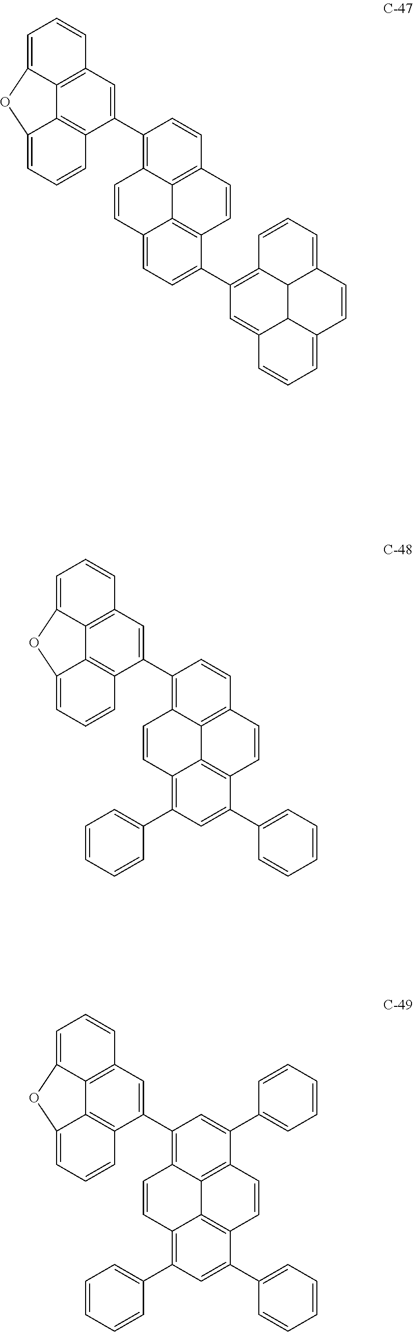 [SMILES: C1=CC2=CC(c3ccc4ccc5c(-c6cc7cccc8oc9cccc6c9c78)ccc6ccc3c4c65)=C3C=CC=C4C=CC(=C1)C2C43 ; c1ccc(-c2cc(-c3ccccc3)c3ccc4c(-c5cc6cccc7oc8cccc5c8c67)ccc5ccc2c3c54)cc1 ; c1ccc(-c2cc(-c3ccccc3)c3ccc4c(-c5cc6cccc7oc8cccc5c8c67)cc(-c5ccccc5)c5ccc2c3c54)cc1]

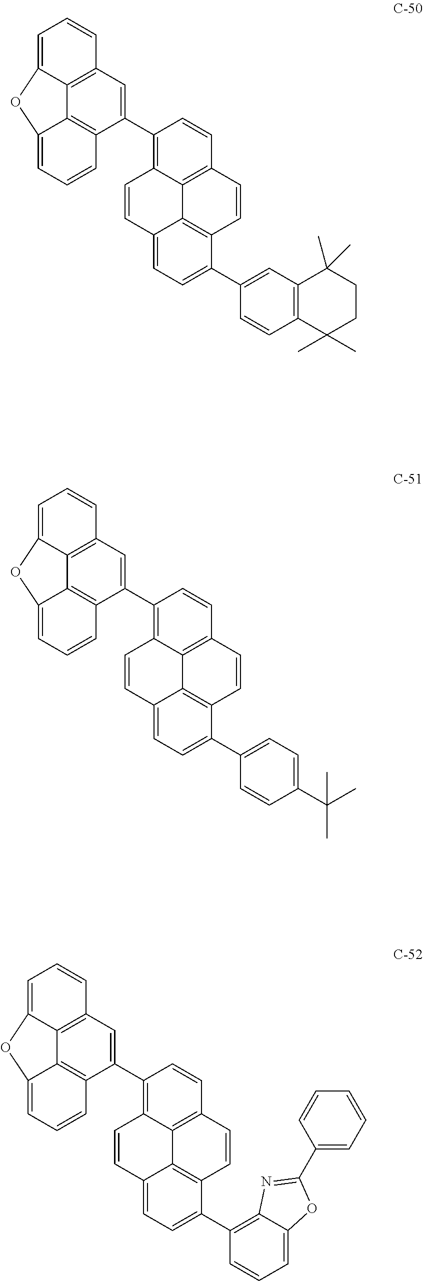 [SMILES: CC1(C)CCC(C)(C)c2cc(-c3ccc4ccc5c(-c6cc7cccc8oc9cccc6c9c78)ccc6ccc3c4c65)ccc21 ; CC(C)(C)c1ccc(-c2ccc3ccc4c(-c5cc6cccc7oc8cccc5c8c67)ccc5ccc2c3c54)cc1 ; c1ccc(-c2nc3c(-c4ccc5ccc6c(-c7cc8cccc9oc%10cccc7c%10c89)ccc7ccc4c5c76)cccc3o2)cc1]

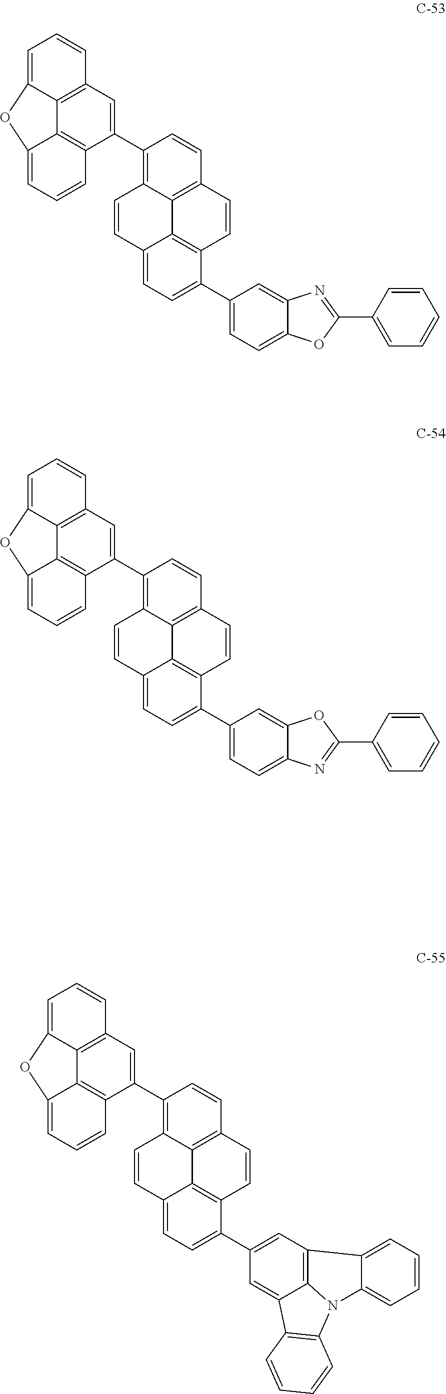 [SMILES: c1ccc(-c2nc3cc(-c4ccc5ccc6c(-c7cc8cccc9oc%10cccc7c%10c89)ccc7ccc4c5c76)ccc3o2)cc1 ; c1ccc(-c2nc3ccc(-c4ccc5ccc6c(-c7cc8cccc9oc%10cccc7c%10c89)ccc7ccc4c5c76)cc3o2)cc1 ; c1cc2cc(-c3ccc4ccc5c(-c6cc7c8ccccc8n8c9ccccc9c(c6)c78)ccc6ccc3c4c65)c3cccc4oc(c1)c2c43]

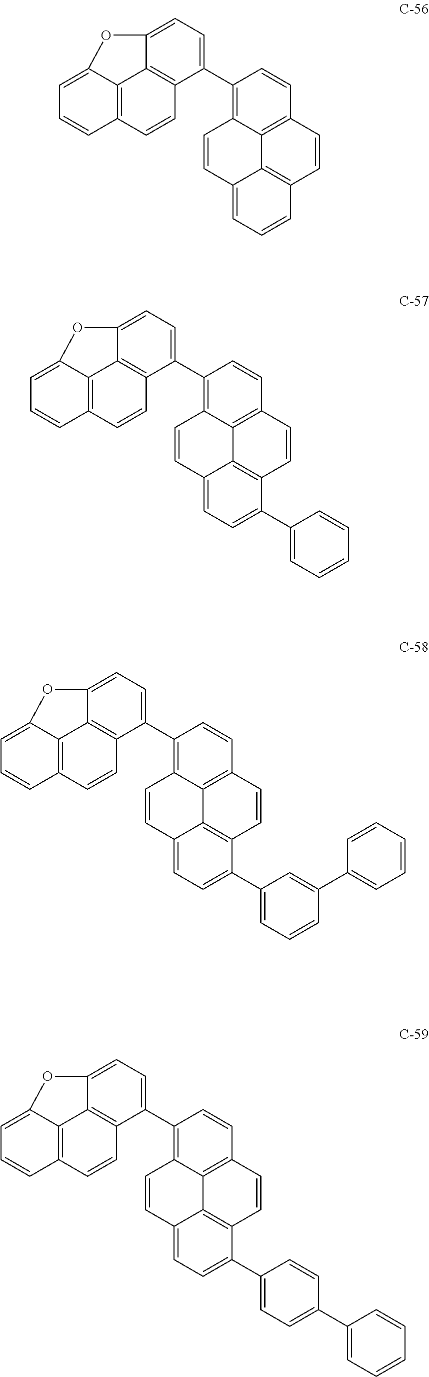 [SMILES: c1cc2ccc3ccc(-c4ccc5oc6cccc7ccc4c5c76)c4ccc(c1)c2c34 ; c1ccc(-c2ccc3ccc4c(-c5ccc6oc7cccc8ccc5c6c87)ccc5ccc2c3c54)cc1 ; c1ccc(-c2cccc(-c3ccc4ccc5c(-c6ccc7oc8cccc9ccc6c7c98)ccc6ccc3c4c65)c2)cc1 ; c1ccc(-c2ccc(-c3ccc4ccc5c(-c6ccc7oc8cccc9ccc6c7c98)ccc6ccc3c4c65)cc2)cc1]

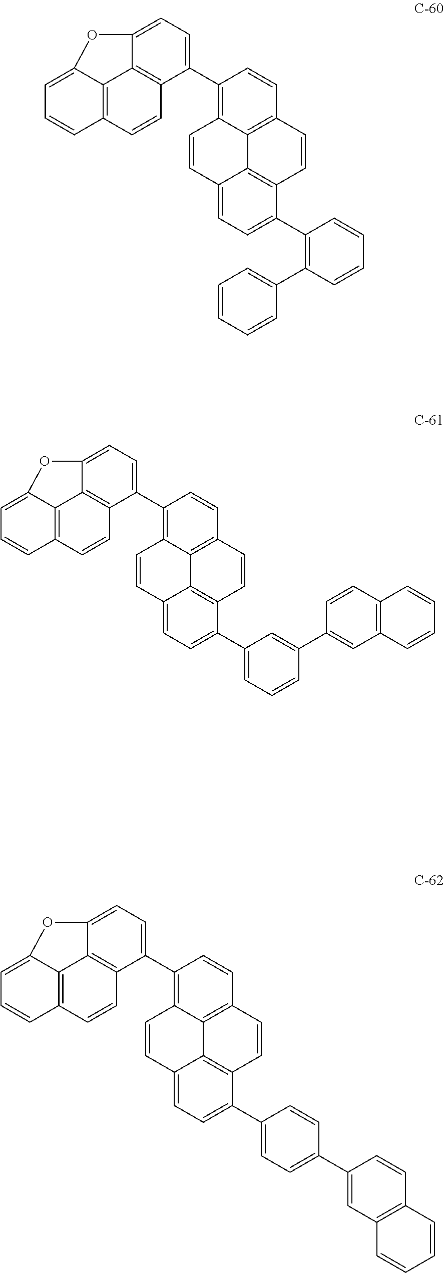 [SMILES: c1ccc(-c2ccccc2-c2ccc3ccc4c(-c5ccc6oc7cccc8ccc5c6c87)ccc5ccc2c3c54)cc1 ; c1cc(-c2ccc3ccccc3c2)cc(-c2ccc3ccc4c(-c5ccc6oc7cccc8ccc5c6c87)ccc5ccc2c3c54)c1 ; c1ccc2cc(-c3ccc(-c4ccc5ccc6c(-c7ccc8oc9cccc%10ccc7c8c%109)ccc7ccc4c5c76)cc3)ccc2c1]

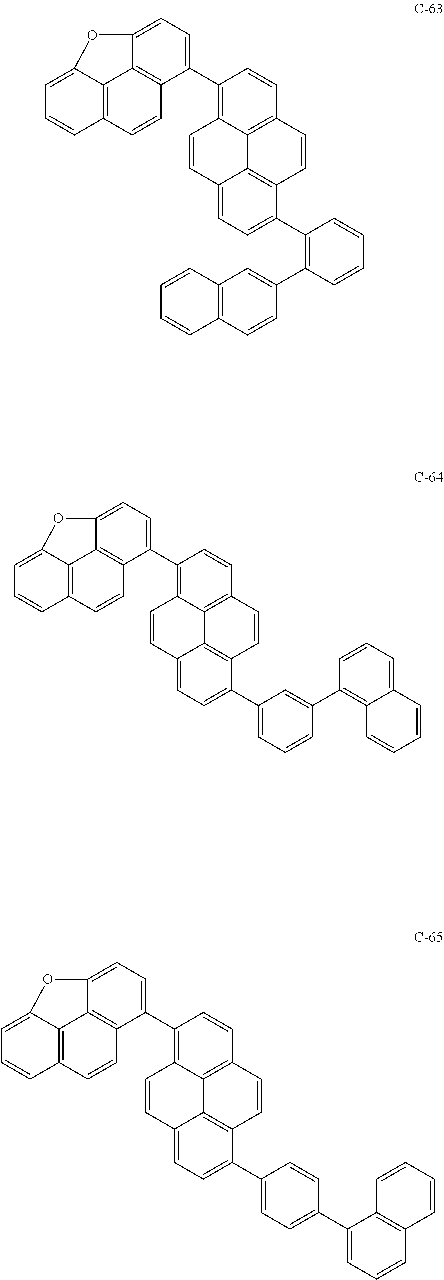 [SMILES: c1ccc(-c2ccc3ccc4c(-c5ccc6oc7cccc8ccc5c6c87)ccc5ccc2c3c54)c(-c2ccc3ccccc3c2)c1 ; c1cc(-c2cccc3ccccc23)cc(-c2ccc3ccc4c(-c5ccc6oc7cccc8ccc5c6c87)ccc5ccc2c3c54)c1 ; c1ccc2c(-c3ccc(-c4ccc5ccc6c(-c7ccc8oc9cccc%10ccc7c8c%109)ccc7ccc4c5c76)cc3)cccc2c1]

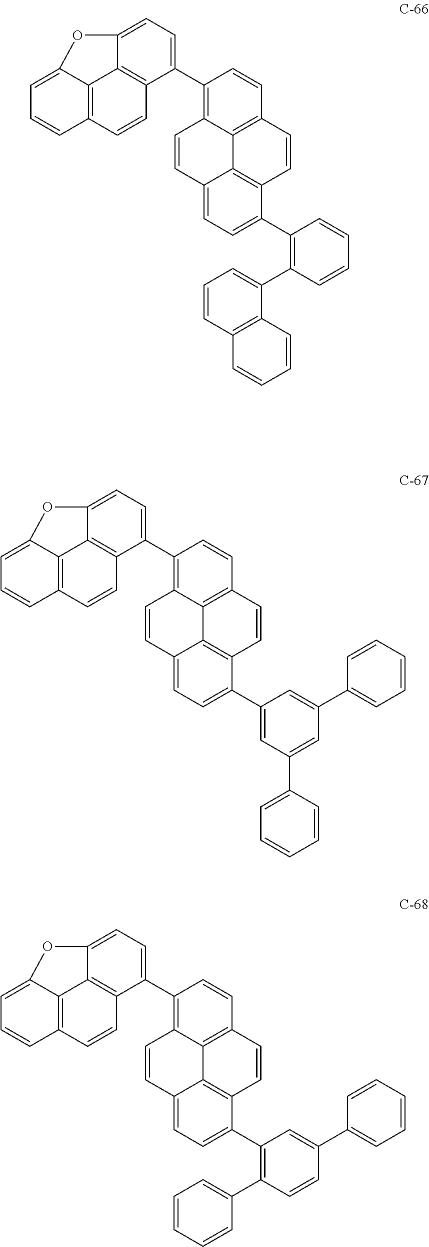 [SMILES: c1ccc(-c2ccc3ccc4c(-c5ccc6oc7cccc8ccc5c6c87)ccc5ccc2c3c54)c(-c2cccc3ccccc23)c1 ; c1ccc(-c2cc(-c3ccccc3)cc(-c3ccc4ccc5c(-c6ccc7oc8cccc9ccc6c7c98)ccc6ccc3c4c65)c2)cc1 ; c1ccc(-c2ccc(-c3ccccc3)c(-c3ccc4ccc5c(-c6ccc7oc8cccc9ccc6c7c98)ccc6ccc3c4c65)c2)cc1]

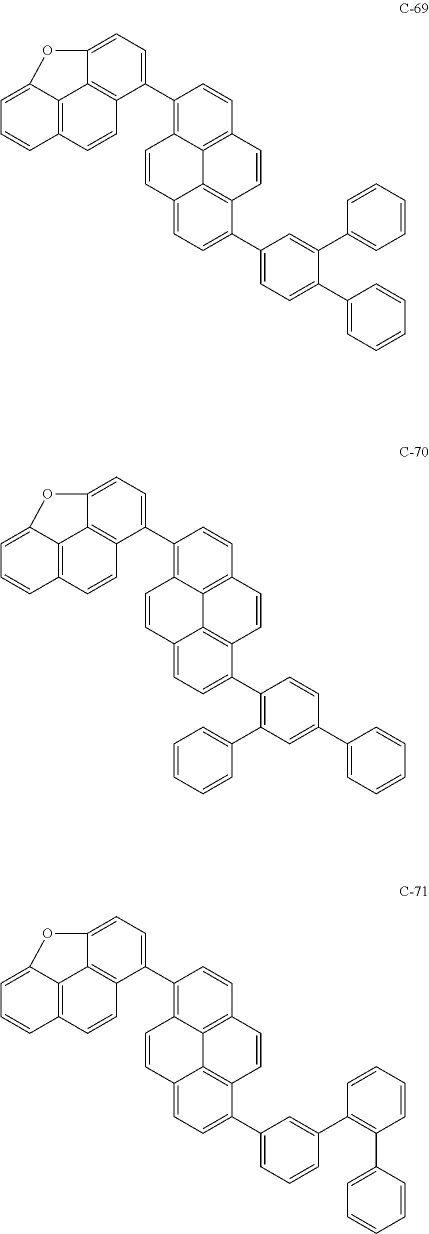 [SMILES: c1ccc(-c2ccc(-c3ccc4ccc5c(-c6ccc7oc8cccc9ccc6c7c98)ccc6ccc3c4c65)cc2-c2ccccc2)cc1 ; c1ccc(-c2ccc(-c3ccc4ccc5c(-c6ccc7oc8cccc9ccc6c7c98)ccc6ccc3c4c65)c(-c3ccccc3)c2)cc1 ; c1ccc(-c2ccccc2-c2cccc(-c3ccc4ccc5c(-c6ccc7oc8cccc9ccc6c7c98)ccc6ccc3c4c65)c2)cc1]

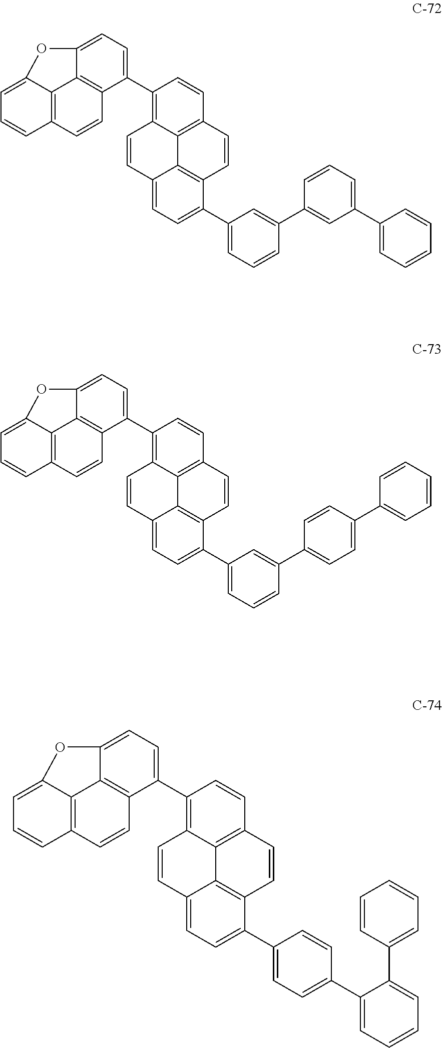 [SMILES: c1ccc(-c2cccc(-c3cccc(-c4ccc5ccc6c(-c7ccc8oc9cccc%10ccc7c8c%109)ccc7ccc4c5c76)c3)c2)cc1 ; c1ccc(-c2ccc(-c3cccc(-c4ccc5ccc6c(-c7ccc8oc9cccc%10ccc7c8c%109)ccc7ccc4c5c76)c3)cc2)cc1 ; c1ccc(-c2ccccc2-c2ccc(-c3ccc4ccc5c(-c6ccc7oc8cccc9ccc6c7c98)ccc6ccc3c4c65)cc2)cc1]

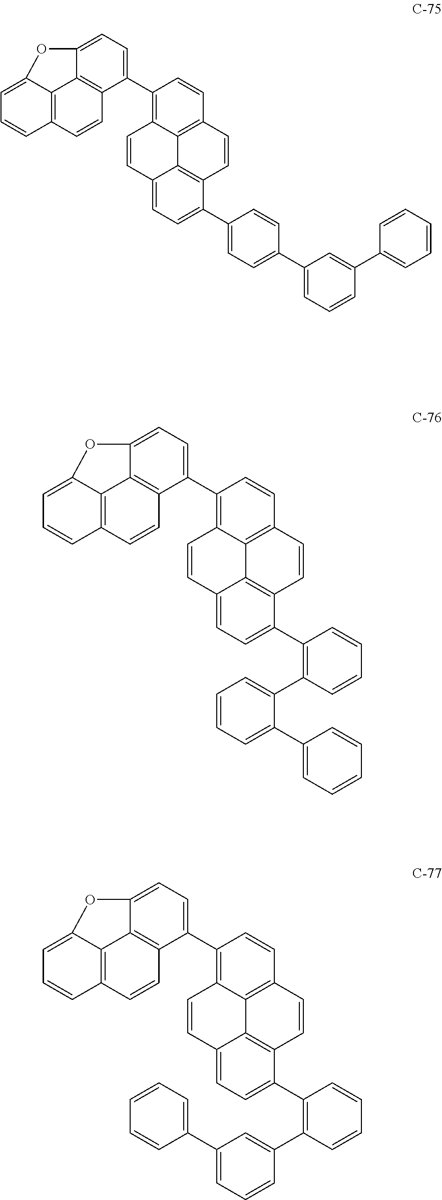 [SMILES: c1ccc(-c2cccc(-c3ccc(-c4ccc5ccc6c(-c7ccc8oc9cccc%10ccc7c8c%109)ccc7ccc4c5c76)cc3)c2)cc1 ; c1ccc(-c2ccccc2-c2ccccc2-c2ccc3ccc4c(-c5ccc6oc7cccc8ccc5c6c87)ccc5ccc2c3c54)cc1 ; c1ccc(-c2cccc(-c3ccccc3-c3ccc4ccc5c(-c6ccc7oc8cccc9ccc6c7c98)ccc6ccc3c4c65)c2)cc1]

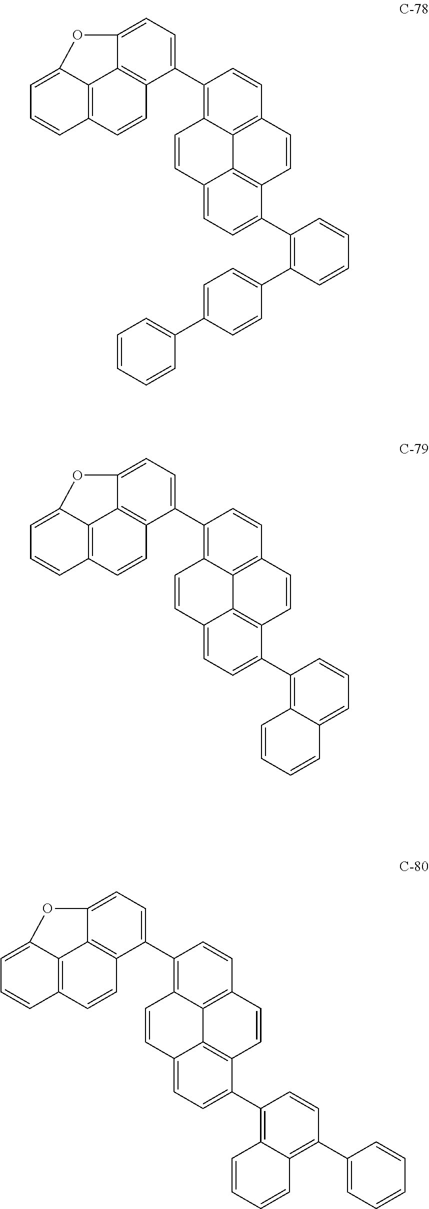 [SMILES: c1ccc(-c2ccc(-c3ccccc3-c3ccc4ccc5c(-c6ccc7oc8cccc9ccc6c7c98)ccc6ccc3c4c65)cc2)cc1 ; c1ccc2c(-c3ccc4ccc5c(-c6ccc7oc8cccc9ccc6c7c98)ccc6ccc3c4c65)cccc2c1 ; c1ccc(-c2ccc(-c3ccc4ccc5c(-c6ccc7oc8cccc9ccc6c7c98)ccc6ccc3c4c65)c3ccccc23)cc1]

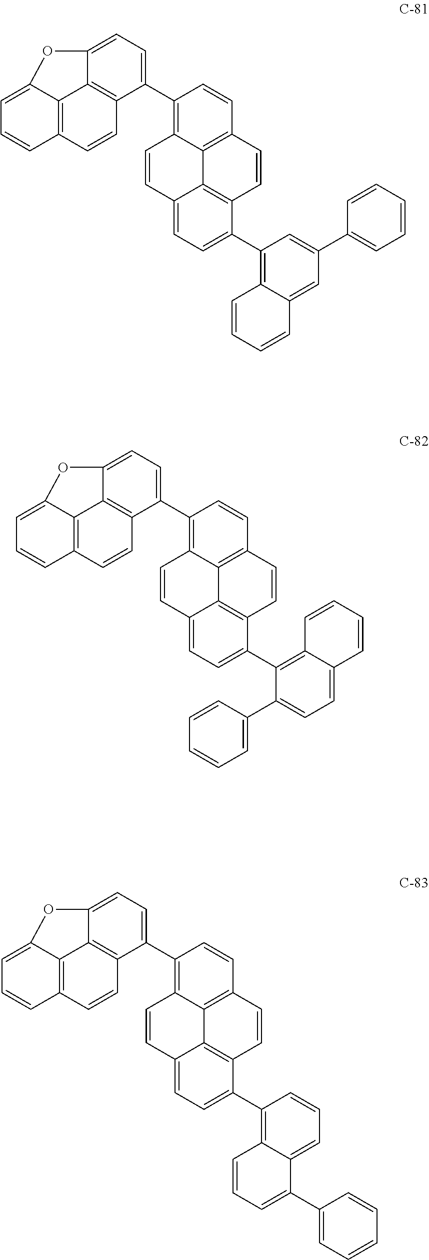 [SMILES: c1ccc(-c2cc(-c3ccc4ccc5c(-c6ccc7oc8cccc9ccc6c7c98)ccc6ccc3c4c65)c3ccccc3c2)cc1 ; c1ccc(-c2ccc3ccccc3c2-c2ccc3ccc4c(-c5ccc6oc7cccc8ccc5c6c87)ccc5ccc2c3c54)cc1 ; c1ccc(-c2cccc3c(-c4ccc5ccc6c(-c7ccc8oc9cccc%10ccc7c8c%109)ccc7ccc4c5c76)cccc23)cc1]

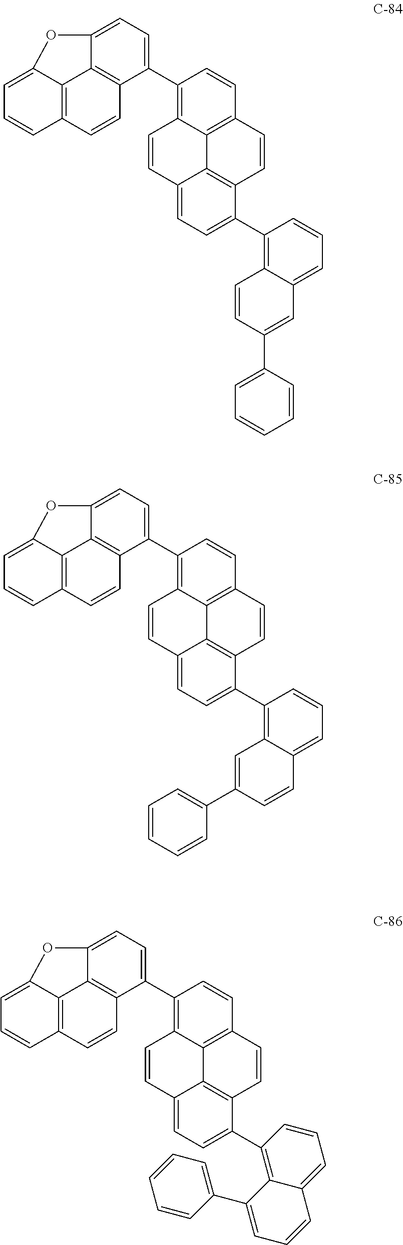 [SMILES: c1ccc(-c2ccc3c(-c4ccc5ccc6c(-c7ccc8oc9cccc%10ccc7c8c%109)ccc7ccc4c5c76)cccc3c2)cc1 ; c1ccc(-c2ccc3cccc(-c4ccc5ccc6c(-c7ccc8oc9cccc%10ccc7c8c%109)ccc7ccc4c5c76)c3c2)cc1 ; c1ccc(-c2cccc3cccc(-c4ccc5ccc6c(-c7ccc8oc9cccc%10ccc7c8c%109)ccc7ccc4c5c76)c23)cc1]

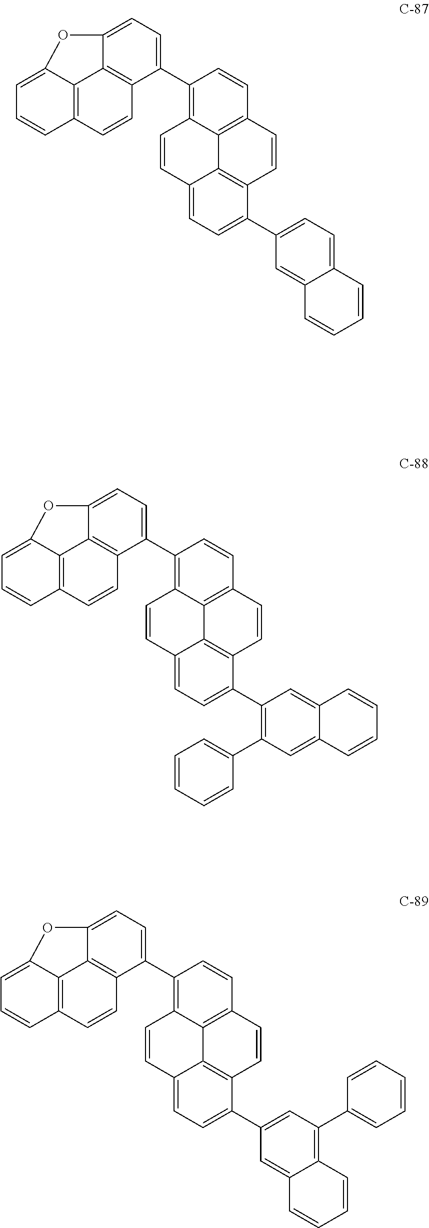 [SMILES: c1ccc2cc(-c3ccc4ccc5c(-c6ccc7oc8cccc9ccc6c7c98)ccc6ccc3c4c65)ccc2c1 ; c1ccc(-c2cc3ccccc3cc2-c2ccc3ccc4c(-c5ccc6oc7cccc8ccc5c6c87)ccc5ccc2c3c54)cc1 ; c1ccc(-c2cc(-c3ccc4ccc5c(-c6ccc7oc8cccc9ccc6c7c98)ccc6ccc3c4c65)cc3ccccc23)cc1]

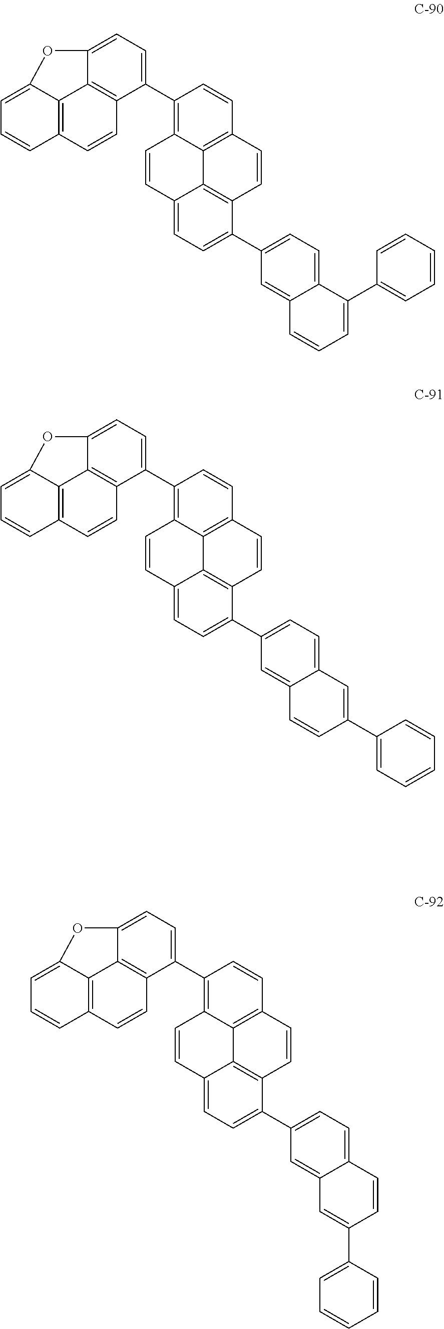 [SMILES: c1ccc(-c2cccc3cc(-c4ccc5ccc6c(-c7ccc8oc9cccc%10ccc7c8c%109)ccc7ccc4c5c76)ccc23)cc1 ; c1ccc(-c2ccc3cc(-c4ccc5ccc6c(-c7ccc8oc9cccc%10ccc7c8c%109)ccc7ccc4c5c76)ccc3c2)cc1 ; c1ccc(-c2ccc3ccc(-c4ccc5ccc6c(-c7ccc8oc9cccc%10ccc7c8c%109)ccc7ccc4c5c76)cc3c2)cc1]

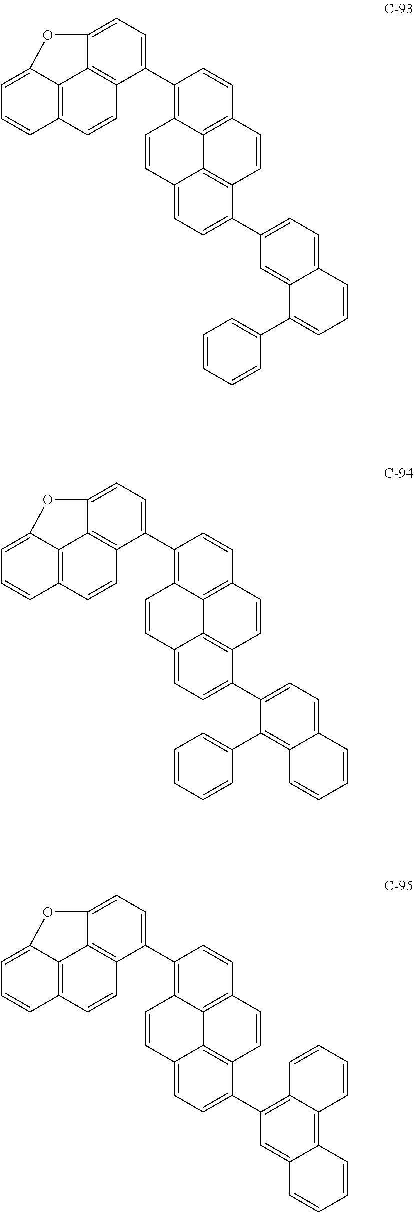 [SMILES: c1ccc(-c2cccc3ccc(-c4ccc5ccc6c(-c7ccc8oc9cccc%10ccc7c8c%109)ccc7ccc4c5c76)cc23)cc1 ; c1ccc(-c2c(-c3ccc4ccc5c(-c6ccc7oc8cccc9ccc6c7c98)ccc6ccc3c4c65)ccc3ccccc23)cc1 ; c1ccc2c(c1)cc(-c1ccc3ccc4c(-c5ccc6oc7cccc8ccc5c6c87)ccc5ccc1c3c54)c1ccccc12]

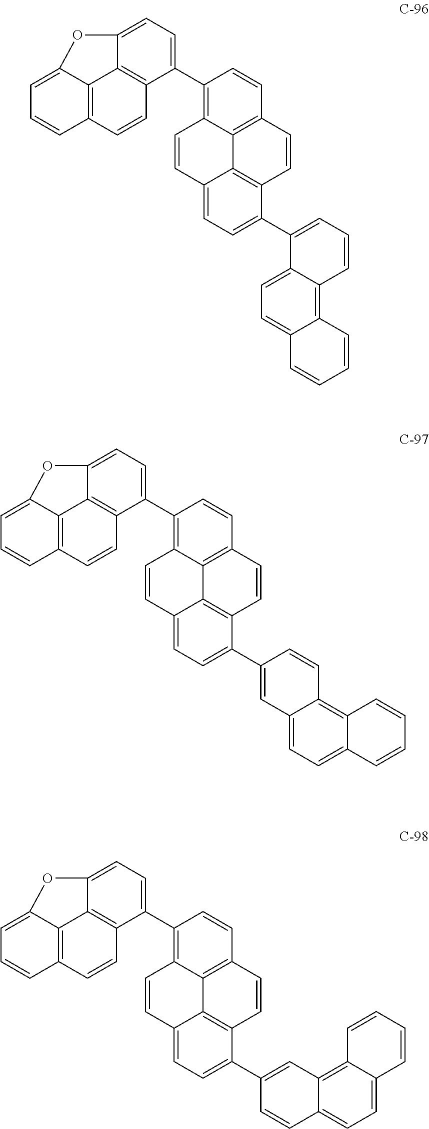 [SMILES: c1ccc2c(c1)ccc1c(-c3ccc4ccc5c(-c6ccc7oc8cccc9ccc6c7c98)ccc6ccc3c4c65)cccc12 ; c1ccc2c(c1)ccc1cc(-c3ccc4ccc5c(-c6ccc7oc8cccc9ccc6c7c98)ccc6ccc3c4c65)ccc12 ; c1ccc2c(c1)ccc1ccc(-c3ccc4ccc5c(-c6ccc7oc8cccc9ccc6c7c98)ccc6ccc3c4c65)cc12]

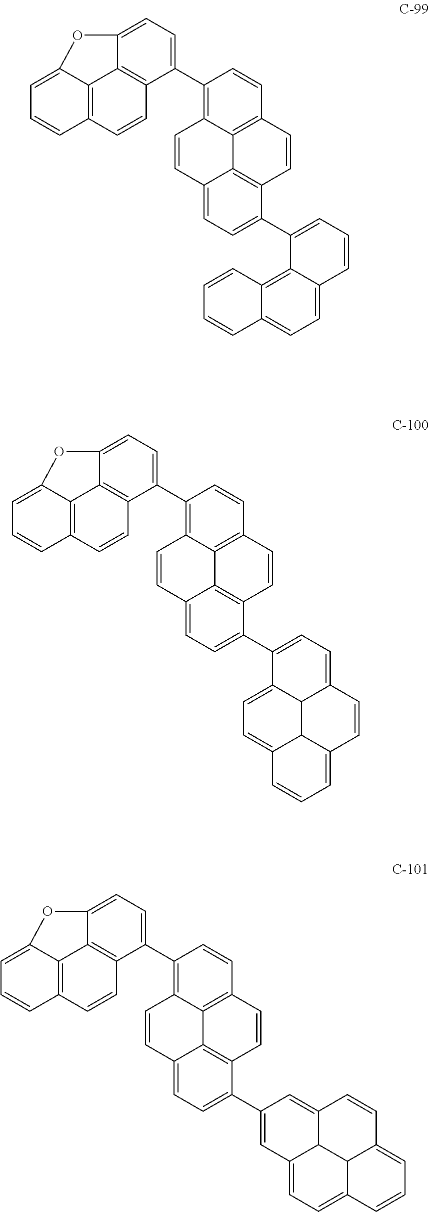 [SMILES: c1ccc2c(c1)ccc1cccc(-c3ccc4ccc5c(-c6ccc7oc8cccc9ccc6c7c98)ccc6ccc3c4c65)c12 ; C1=CC2=CC=C3C(c4ccc5ccc6c(-c7ccc8oc9cccc%10ccc7c8c%109)ccc7ccc4c5c76)=CC=C4C=CC(=C1)C2C43 ; C1=CC2=CC=C3C=C(c4ccc5ccc6c(-c7ccc8oc9cccc%10ccc7c8c%109)ccc7ccc4c5c76)C=C4C=CC(=C1)C2C34]

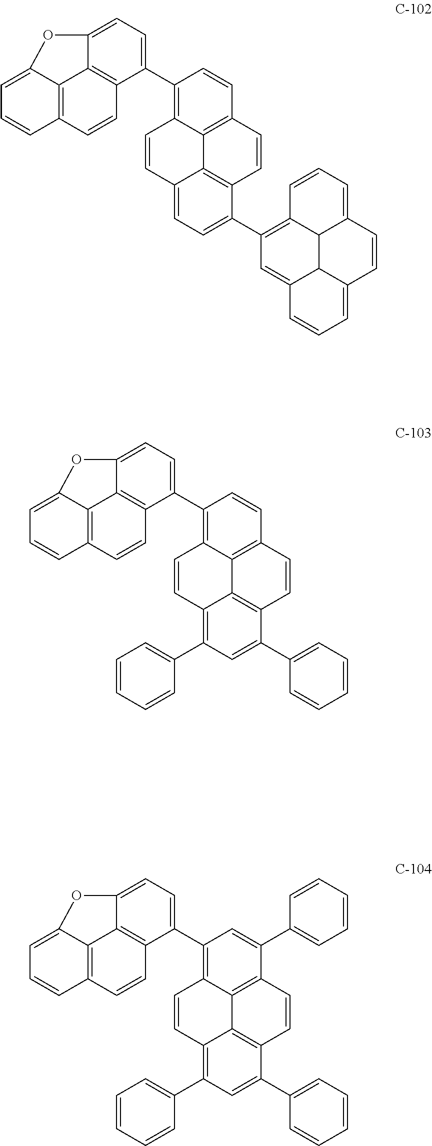 [SMILES: C1=CC2=CC(c3ccc4ccc5c(-c6ccc7oc8cccc9ccc6c7c98)ccc6ccc3c4c65)=C3C=CC=C4C=CC(=C1)C2C43 ; c1ccc(-c2cc(-c3ccccc3)c3ccc4c(-c5ccc6oc7cccc8ccc5c6c87)ccc5ccc2c3c54)cc1 ; c1ccc(-c2cc(-c3ccccc3)c3ccc4c(-c5ccc6oc7cccc8ccc5c6c87)cc(-c5ccccc5)c5ccc2c3c54)cc1]

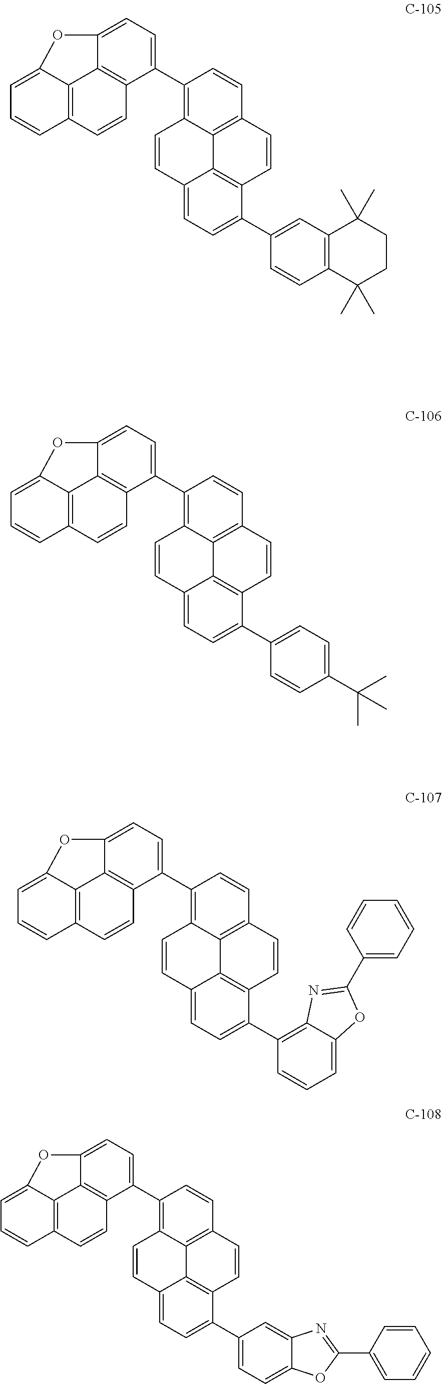 [SMILES: CC1(C)CCC(C)(C)c2cc(-c3ccc4ccc5c(-c6ccc7oc8cccc9ccc6c7c98)ccc6ccc3c4c65)ccc21 ; CC(C)(C)c1ccc(-c2ccc3ccc4c(-c5ccc6oc7cccc8ccc5c6c87)ccc5ccc2c3c54)cc1 ; c1ccc(-c2nc3c(-c4ccc5ccc6c(-c7ccc8oc9cccc%10ccc7c8c%109)ccc7ccc4c5c76)cccc3o2)cc1 ; c1ccc(-c2nc3cc(-c4ccc5ccc6c(-c7ccc8oc9cccc%10ccc7c8c%109)ccc7ccc4c5c76)ccc3o2)cc1]

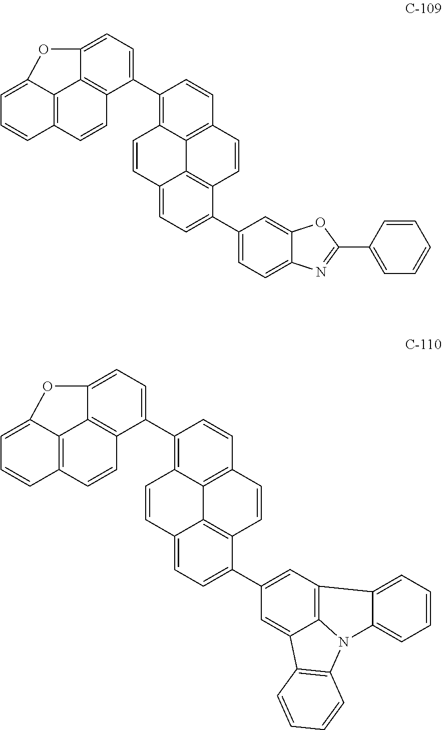 [SMILES: c1ccc(-c2nc3ccc(-c4ccc5ccc6c(-c7ccc8oc9cccc%10ccc7c8c%109)ccc7ccc4c5c76)cc3o2)cc1 ; c1cc2ccc3c(-c4ccc5ccc6c(-c7cc8c9ccccc9n9c%10ccccc%10c(c7)c89)ccc7ccc4c5c76)ccc4oc(c1)c2c43]

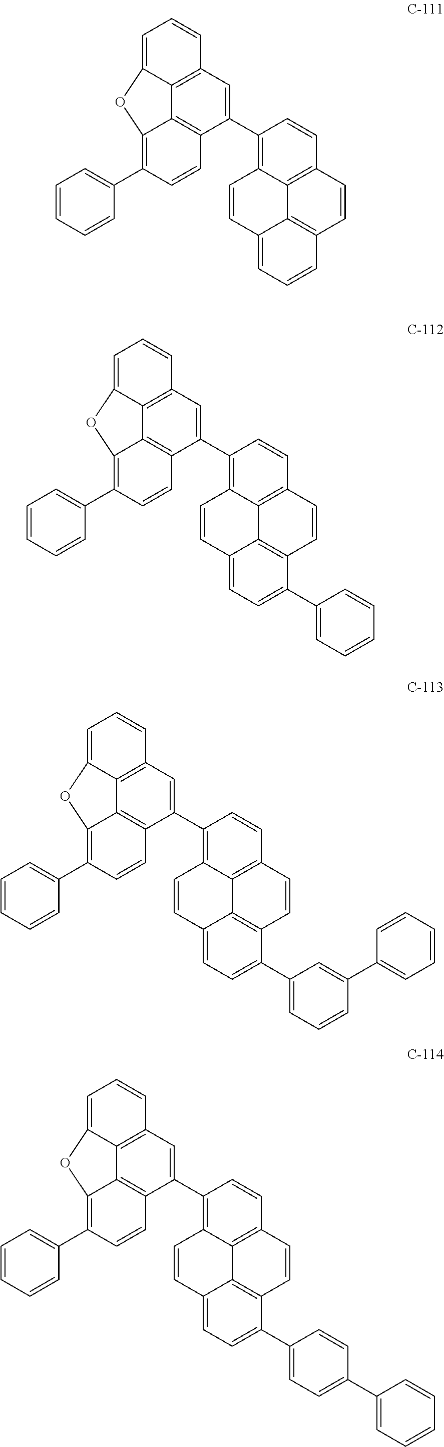 [SMILES: c1ccc(-c2ccc3c(-c4ccc5ccc6cccc7ccc4c5c67)cc4cccc5oc2c3c45)cc1 ; c1ccc(-c2ccc3ccc4c(-c5cc6cccc7oc8c(-c9ccccc9)ccc5c8c67)ccc5ccc2c3c54)cc1 ; c1ccc(-c2cccc(-c3ccc4ccc5c(-c6cc7cccc8oc9c(-c%10ccccc%10)ccc6c9c78)ccc6ccc3c4c65)c2)cc1 ; c1ccc(-c2ccc(-c3ccc4ccc5c(-c6cc7cccc8oc9c(-c%10ccccc%10)ccc6c9c78)ccc6ccc3c4c65)cc2)cc1]

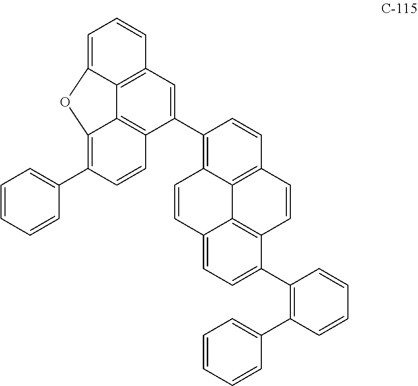 [SMILES: c1ccc(-c2ccccc2-c2ccc3ccc4c(-c5cc6cccc7oc8c(-c9ccccc9)ccc5c8c67)ccc5ccc2c3c54)cc1]

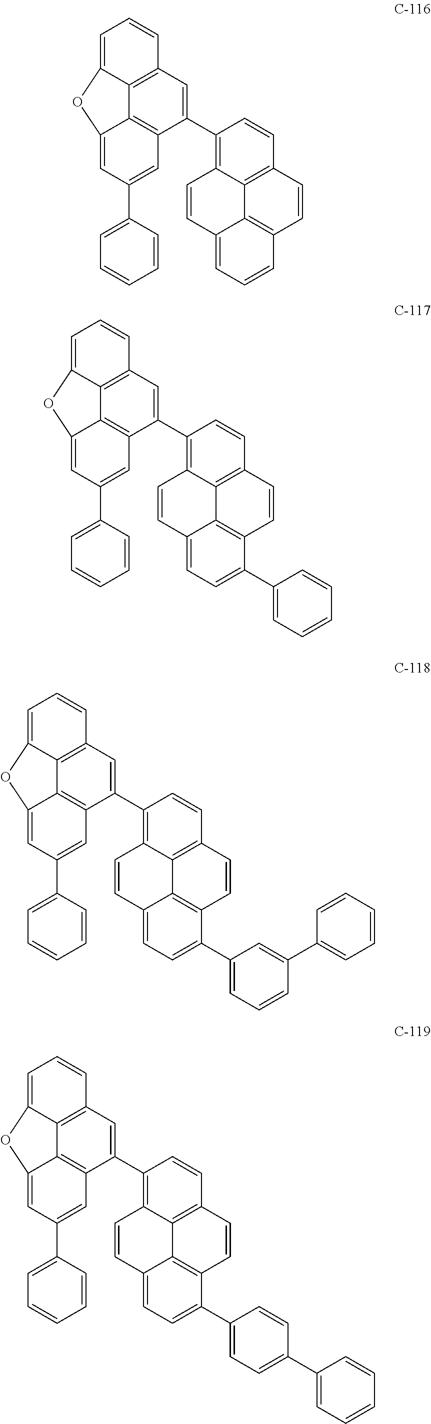

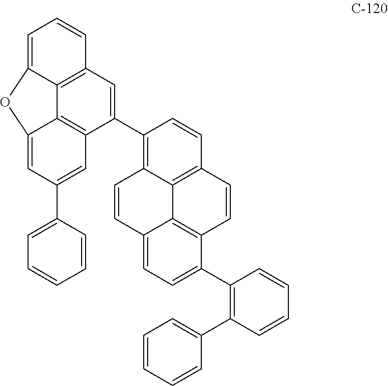 [SMILES: c1ccc(-c2cc3oc4cccc5cc(-c6ccc7ccc8c(-c9ccccc9-c9ccccc9)ccc9ccc6c7c98)c(c2)c3c54)cc1]

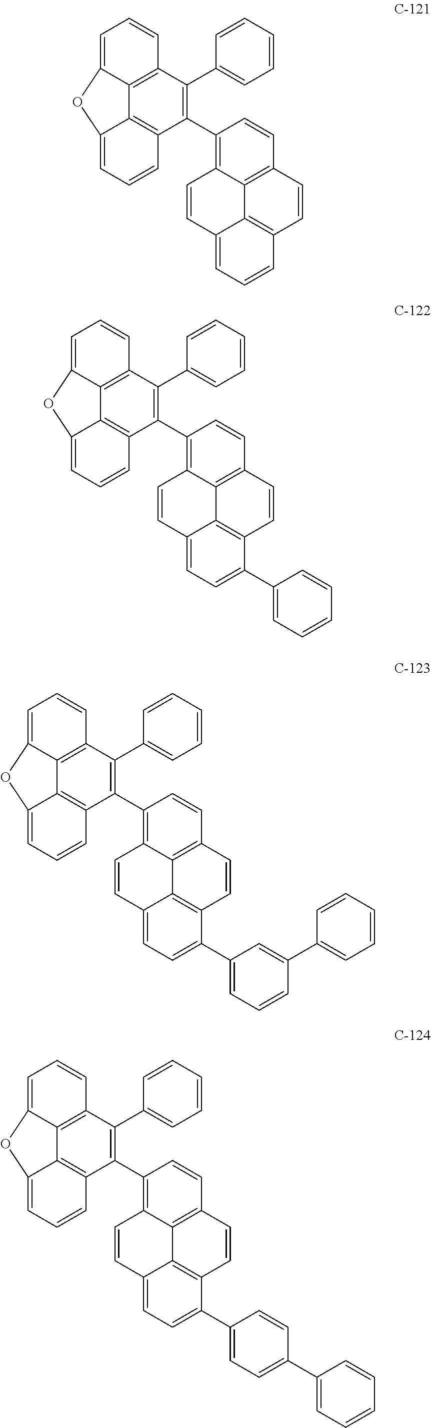

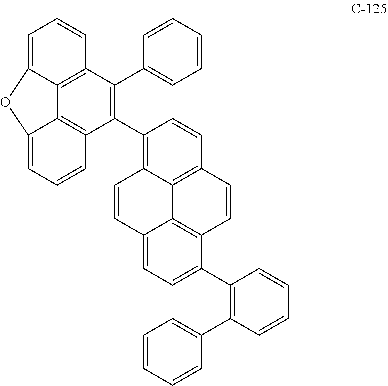 [SMILES: c1ccc(-c2ccccc2-c2ccc3ccc4c(-c5c(-c6ccccc6)c6cccc7oc8cccc5c8c76)ccc5ccc2c3c54)cc1]

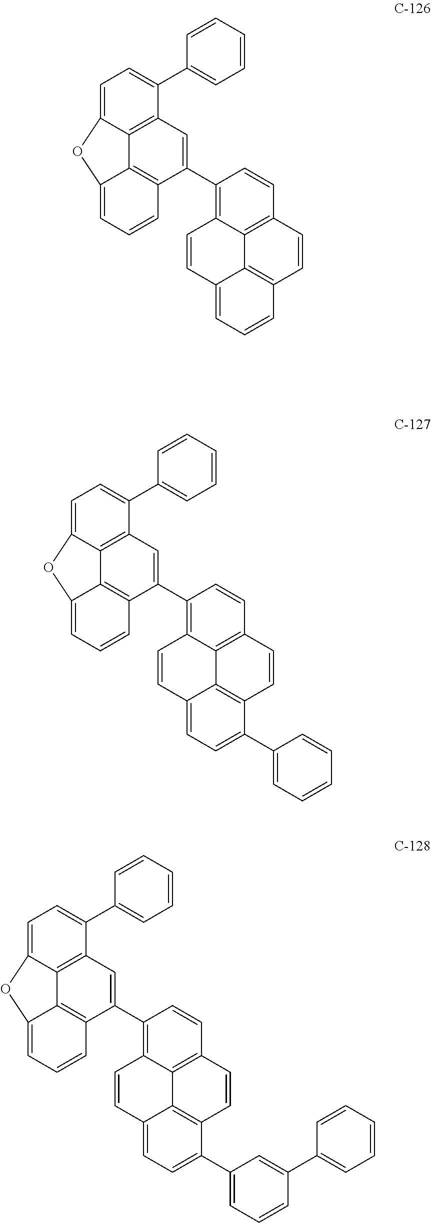 [SMILES: c1ccc(-c2ccc3oc4cccc5c(-c6ccc7ccc8cccc9ccc6c7c89)cc2c3c45)cc1 ; c1ccc(-c2ccc3ccc4c(-c5cc6c(-c7ccccc7)ccc7oc8cccc5c8c76)ccc5ccc2c3c54)cc1 ; c1ccc(-c2cccc(-c3ccc4ccc5c(-c6cc7c(-c8ccccc8)ccc8oc9cccc6c9c87)ccc6ccc3c4c65)c2)cc1]

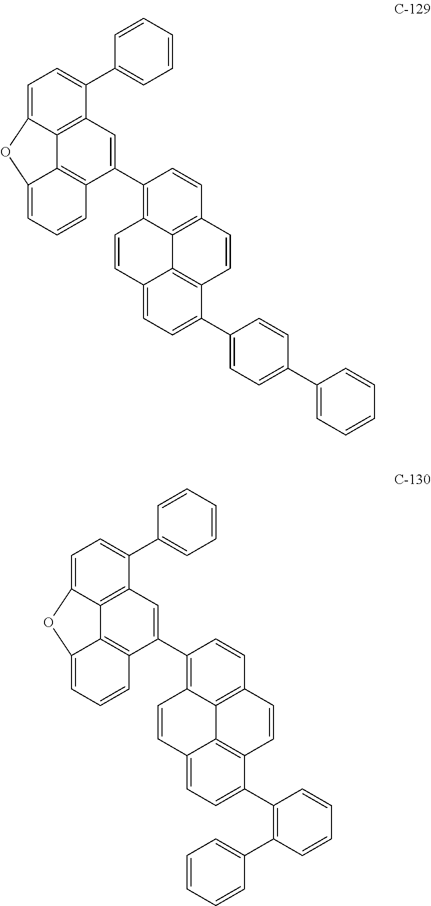 [SMILES: c1ccc(-c2ccc(-c3ccc4ccc5c(-c6cc7c(-c8ccccc8)ccc8oc9cccc6c9c87)ccc6ccc3c4c65)cc2)cc1 ; c1ccc(-c2ccccc2-c2ccc3ccc4c(-c5cc6c(-c7ccccc7)ccc7oc8cccc5c8c76)ccc5ccc2c3c54)cc1]

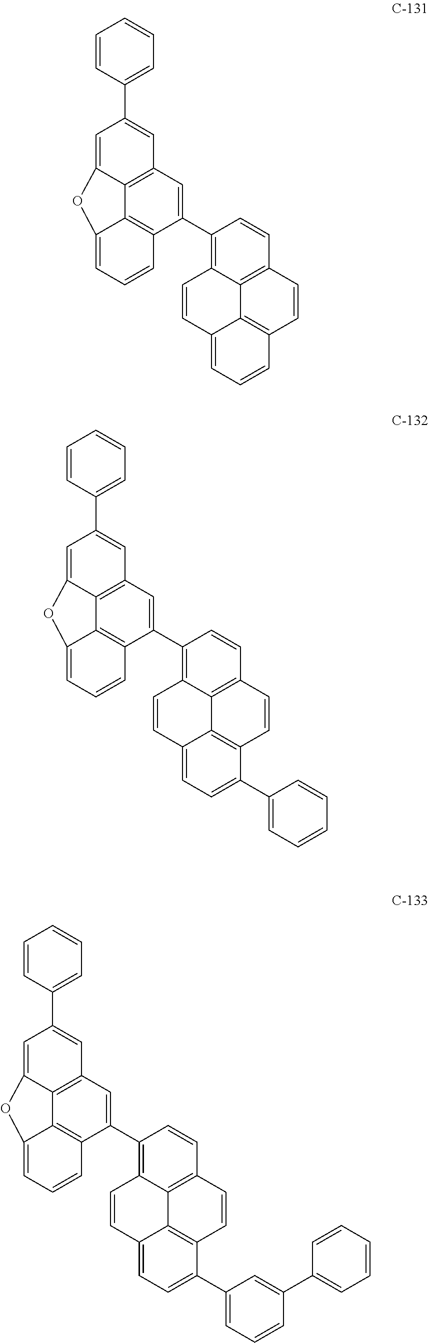 [SMILES: c1ccc(-c2cc3cc(-c4ccc5ccc6cccc7ccc4c5c67)c4cccc5oc(c2)c3c54)cc1 ; c1ccc(-c2cc3cc(-c4ccc5ccc6c(-c7ccccc7)ccc7ccc4c5c76)c4cccc5oc(c2)c3c54)cc1 ; c1ccc(-c2cccc(-c3ccc4ccc5c(-c6cc7cc(-c8ccccc8)cc8oc9cccc6c9c78)ccc6ccc3c4c65)c2)cc1]

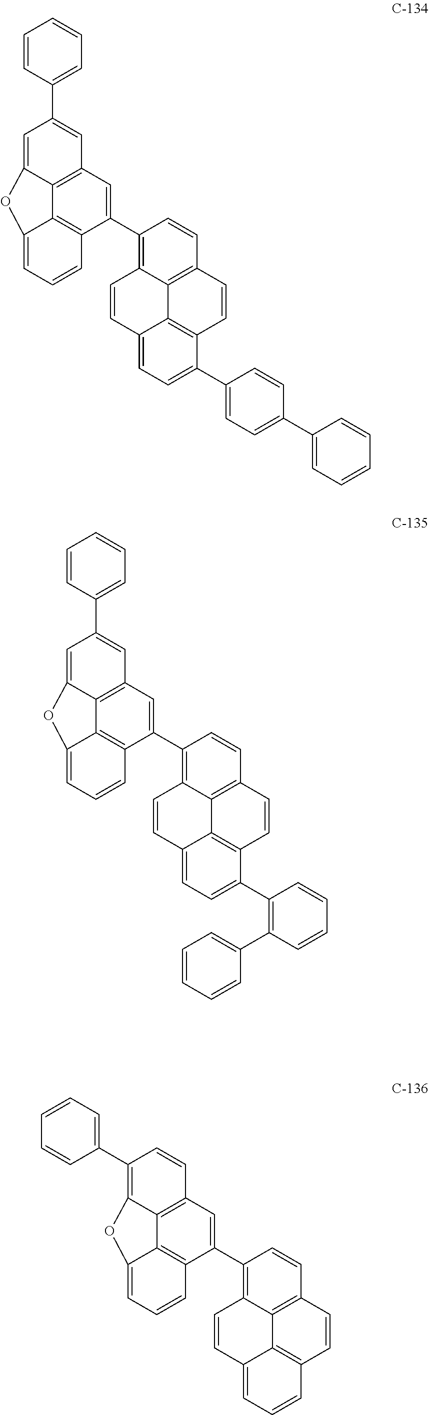 [SMILES: c1ccc(-c2ccc(-c3ccc4ccc5c(-c6cc7cc(-c8ccccc8)cc8oc9cccc6c9c78)ccc6ccc3c4c65)cc2)cc1 ; c1ccc(-c2cc3cc(-c4ccc5ccc6c(-c7ccccc7-c7ccccc7)ccc7ccc4c5c76)c4cccc5oc(c2)c3c54)cc1 ; c1ccc(-c2ccc3cc(-c4ccc5ccc6cccc7ccc4c5c67)c4cccc5oc2c3c54)cc1]

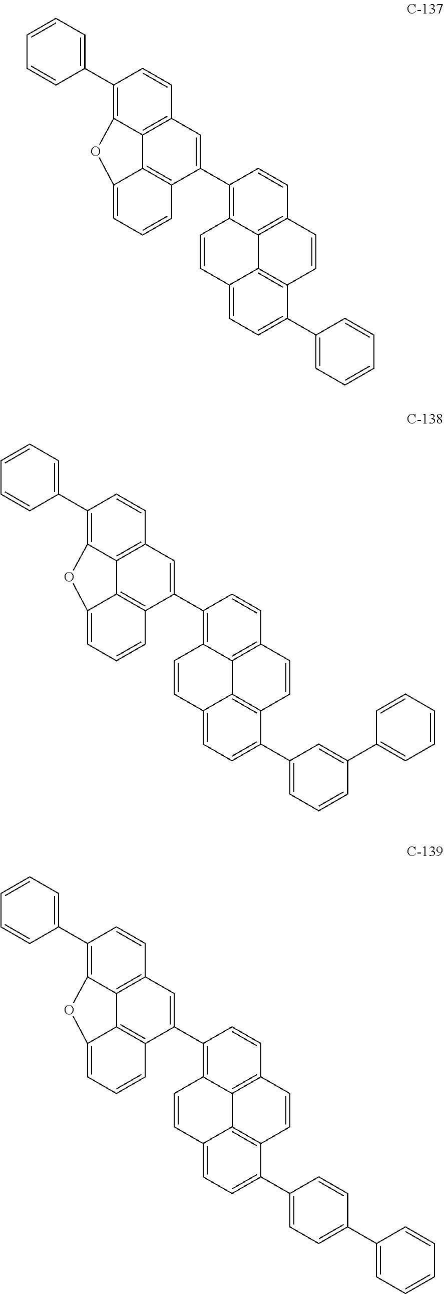 [SMILES: c1ccc(-c2ccc3ccc4c(-c5cc6ccc(-c7ccccc7)c7oc8cccc5c8c67)ccc5ccc2c3c54)cc1 ; c1ccc(-c2cccc(-c3ccc4ccc5c(-c6cc7ccc(-c8ccccc8)c8oc9cccc6c9c78)ccc6ccc3c4c65)c2)cc1 ; c1ccc(-c2ccc(-c3ccc4ccc5c(-c6cc7ccc(-c8ccccc8)c8oc9cccc6c9c78)ccc6ccc3c4c65)cc2)cc1]

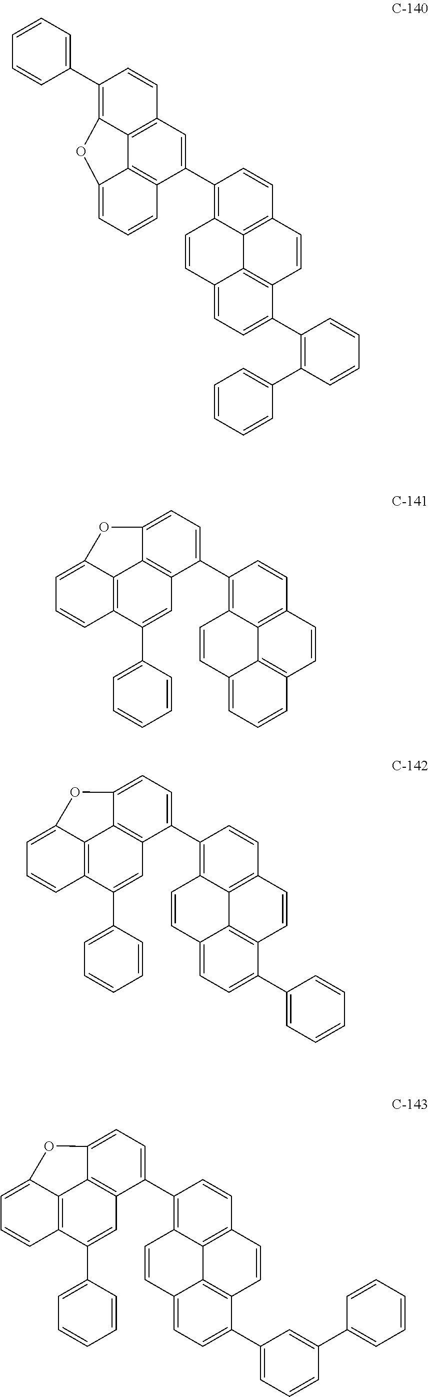 [SMILES: c1ccc(-c2ccccc2-c2ccc3ccc4c(-c5cc6ccc(-c7ccccc7)c7oc8cccc5c8c67)ccc5ccc2c3c54)cc1 ; c1ccc(-c2cc3c(-c4ccc5ccc6cccc7ccc4c5c67)ccc4oc5cccc2c5c43)cc1 ; c1ccc(-c2ccc3ccc4c(-c5ccc6oc7cccc8c(-c9ccccc9)cc5c6c78)ccc5ccc2c3c54)cc1 ; c1ccc(-c2cccc(-c3ccc4ccc5c(-c6ccc7oc8cccc9c(-c%10ccccc%10)cc6c7c89)ccc6ccc3c4c65)c2)cc1]

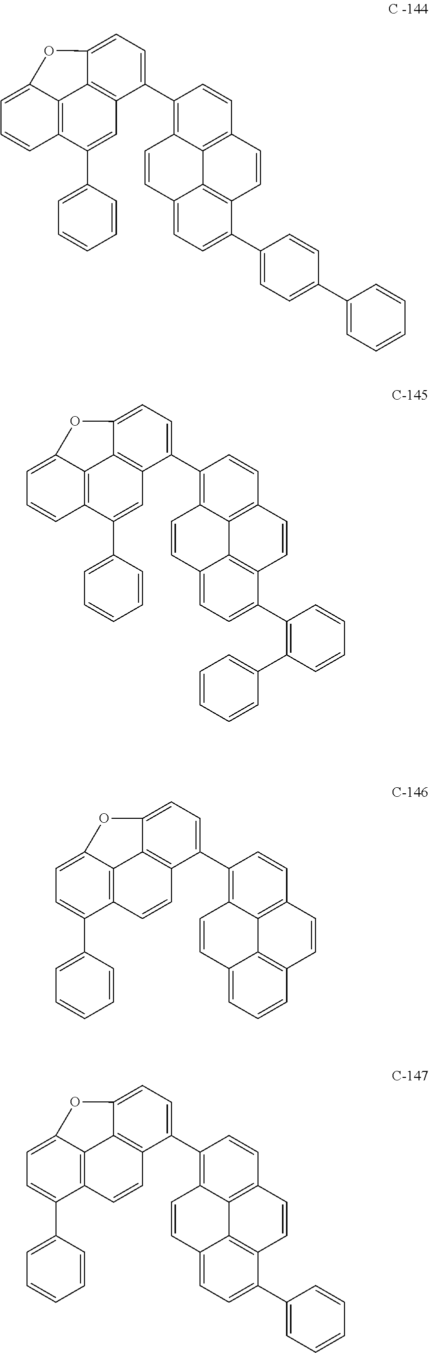 [SMILES: c1ccc(-c2ccc(-c3ccc4ccc5c(-c6ccc7oc8cccc9c(-c%10ccccc%10)cc6c7c89)ccc6ccc3c4c65)cc2)cc1 ; c1ccc(-c2ccccc2-c2ccc3ccc4c(-c5ccc6oc7cccc8c(-c9ccccc9)cc5c6c78)ccc5ccc2c3c54)cc1 ; c1ccc(-c2ccc3oc4ccc(-c5ccc6ccc7cccc8ccc5c6c78)c5ccc2c3c45)cc1 ; c1ccc(-c2ccc3ccc4c(-c5ccc6oc7ccc(-c8ccccc8)c8ccc5c6c78)ccc5ccc2c3c54)cc1]

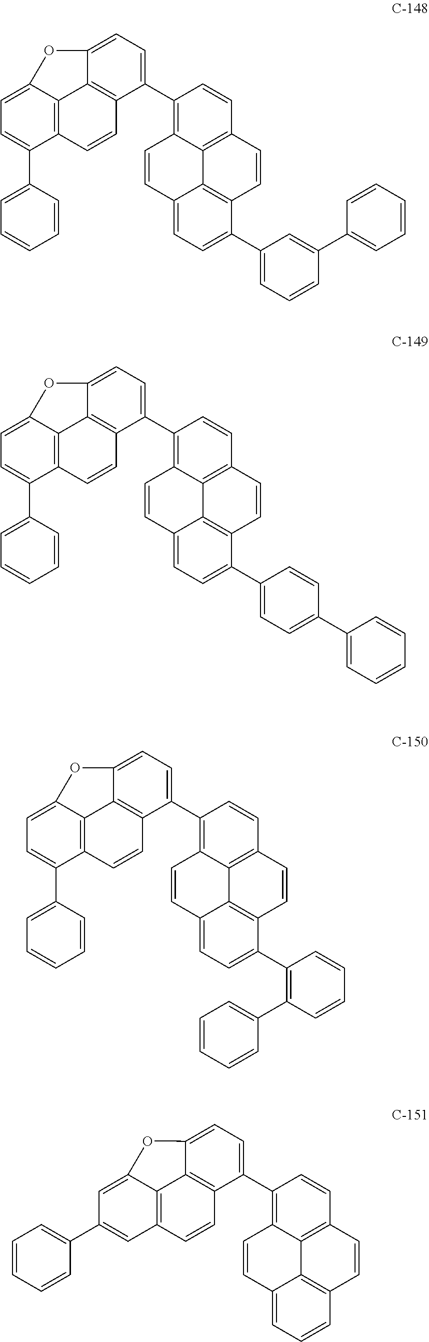 [SMILES: c1ccc(-c2cccc(-c3ccc4ccc5c(-c6ccc7oc8ccc(-c9ccccc9)c9ccc6c7c89)ccc6ccc3c4c65)c2)cc1 ; c1ccc(-c2ccc(-c3ccc4ccc5c(-c6ccc7oc8ccc(-c9ccccc9)c9ccc6c7c89)ccc6ccc3c4c65)cc2)cc1 ; c1ccc(-c2ccccc2-c2ccc3ccc4c(-c5ccc6oc7ccc(-c8ccccc8)c8ccc5c6c78)ccc5ccc2c3c54)cc1 ; c1ccc(-c2cc3ccc4c(-c5ccc6ccc7cccc8ccc5c6c78)ccc5oc(c2)c3c54)cc1]

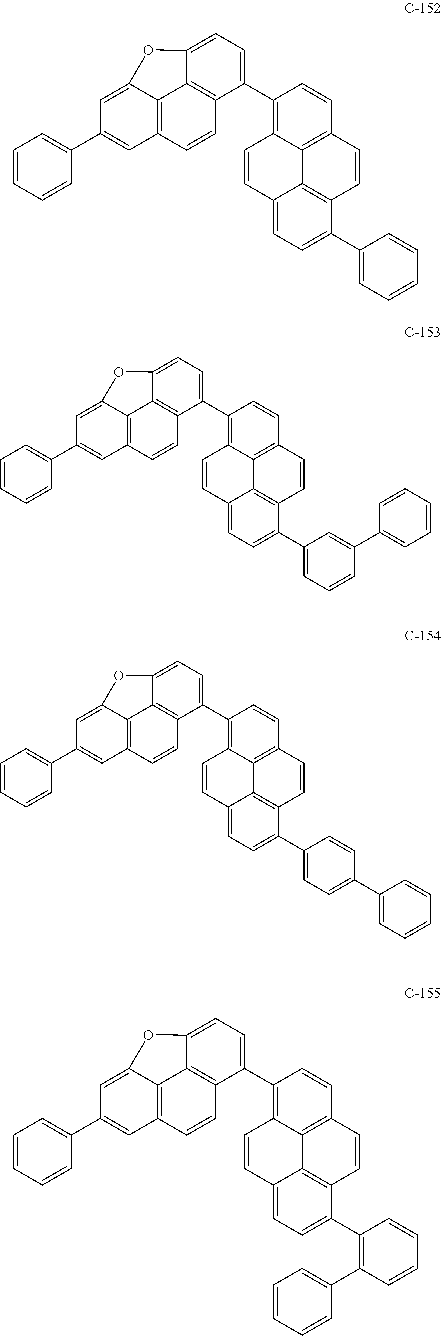 [SMILES: c1ccc(-c2cc3ccc4c(-c5ccc6ccc7c(-c8ccccc8)ccc8ccc5c6c87)ccc5oc(c2)c3c54)cc1 ; c1ccc(-c2cccc(-c3ccc4ccc5c(-c6ccc7oc8cc(-c9ccccc9)cc9ccc6c7c98)ccc6ccc3c4c65)c2)cc1 ; c1ccc(-c2ccc(-c3ccc4ccc5c(-c6ccc7oc8cc(-c9ccccc9)cc9ccc6c7c98)ccc6ccc3c4c65)cc2)cc1 ; c1ccc(-c2cc3ccc4c(-c5ccc6ccc7c(-c8ccccc8-c8ccccc8)ccc8ccc5c6c87)ccc5oc(c2)c3c54)cc1]

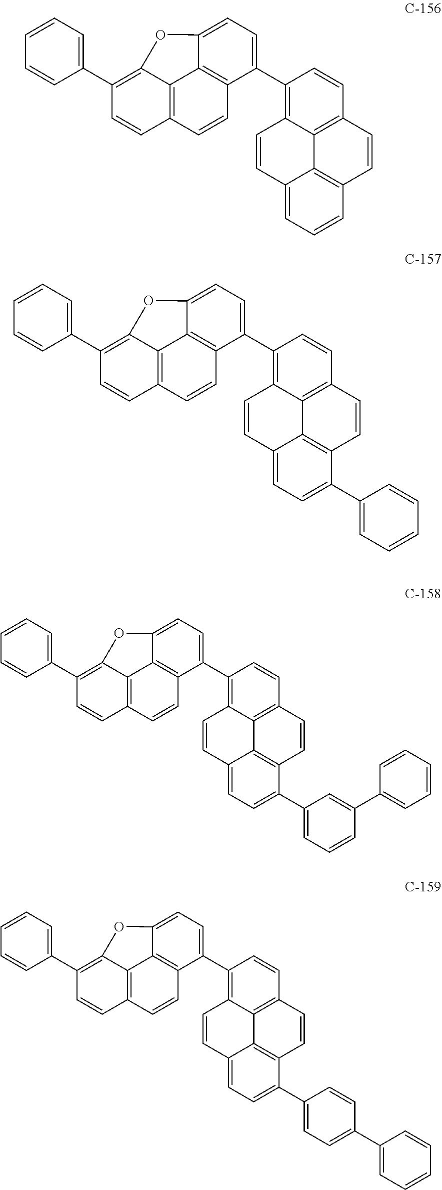 [SMILES: c1ccc(-c2ccc3ccc4c(-c5ccc6ccc7cccc8ccc5c6c78)ccc5oc2c3c54)cc1 ; c1ccc(-c2ccc3ccc4c(-c5ccc6oc7c(-c8ccccc8)ccc8ccc5c6c87)ccc5ccc2c3c54)cc1 ; c1ccc(-c2cccc(-c3ccc4ccc5c(-c6ccc7oc8c(-c9ccccc9)ccc9ccc6c7c98)ccc6ccc3c4c65)c2)cc1 ; c1ccc(-c2ccc(-c3ccc4ccc5c(-c6ccc7oc8c(-c9ccccc9)ccc9ccc6c7c98)ccc6ccc3c4c65)cc2)cc1]

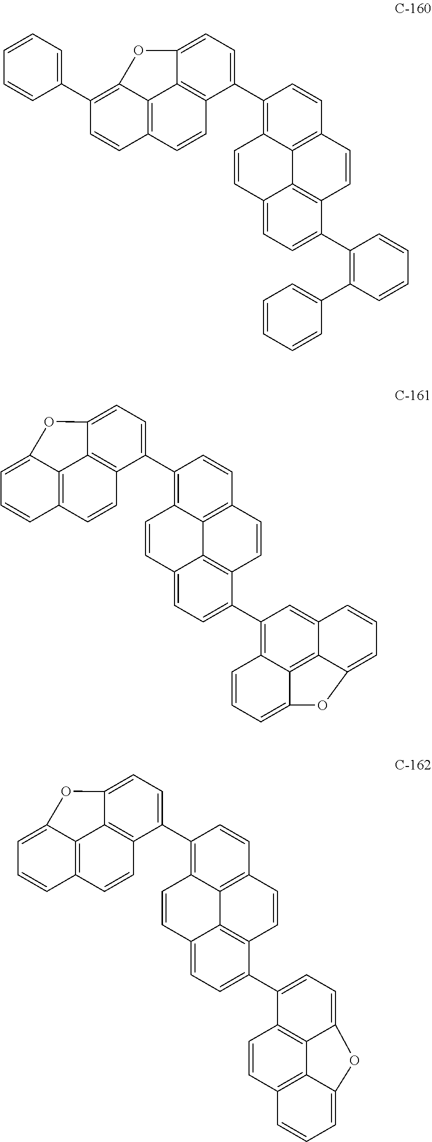 [SMILES: c1ccc(-c2ccccc2-c2ccc3ccc4c(-c5ccc6oc7c(-c8ccccc8)ccc8ccc5c6c87)ccc5ccc2c3c54)cc1 ; c1cc2ccc3c(-c4ccc5ccc6c(-c7cc8cccc9oc%10cccc7c%10c89)ccc7ccc4c5c76)ccc4oc(c1)c2c43 ; c1cc2ccc3c(-c4ccc5ccc6c(-c7ccc8oc9cccc%10ccc7c8c%109)ccc7ccc4c5c76)ccc4oc(c1)c2c43]

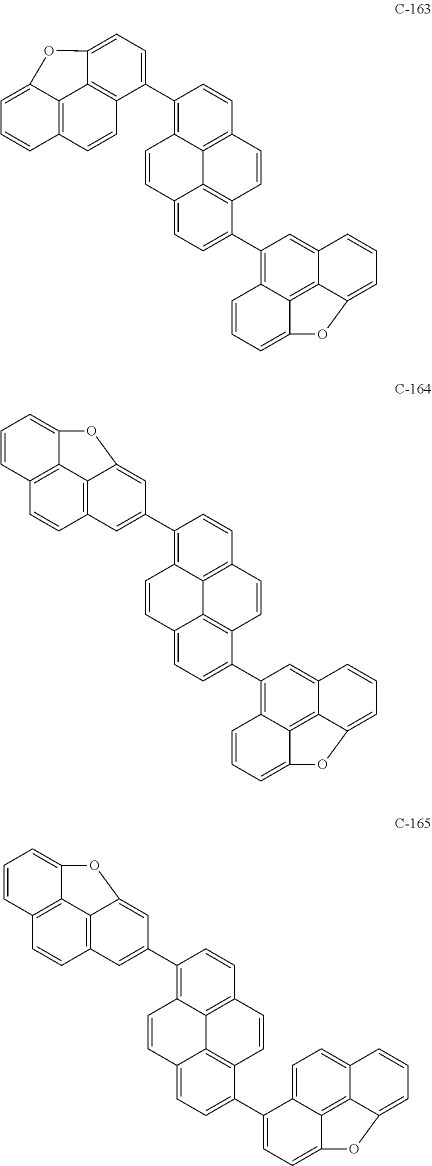 [SMILES: c1cc2ccc3c(-c4ccc5ccc6c(-c7cc8cccc9oc%10cccc7c%10c89)ccc7ccc4c5c76)ccc4oc(c1)c2c43 ; c1cc2ccc3cc(-c4ccc5ccc6c(-c7cc8cccc9oc%10cccc7c%10c89)ccc7ccc4c5c76)cc4oc(c1)c2c34 ; c1cc2ccc3cc(-c4ccc5ccc6c(-c7ccc8oc9cccc%10ccc7c8c%109)ccc7ccc4c5c76)cc4oc(c1)c2c34]

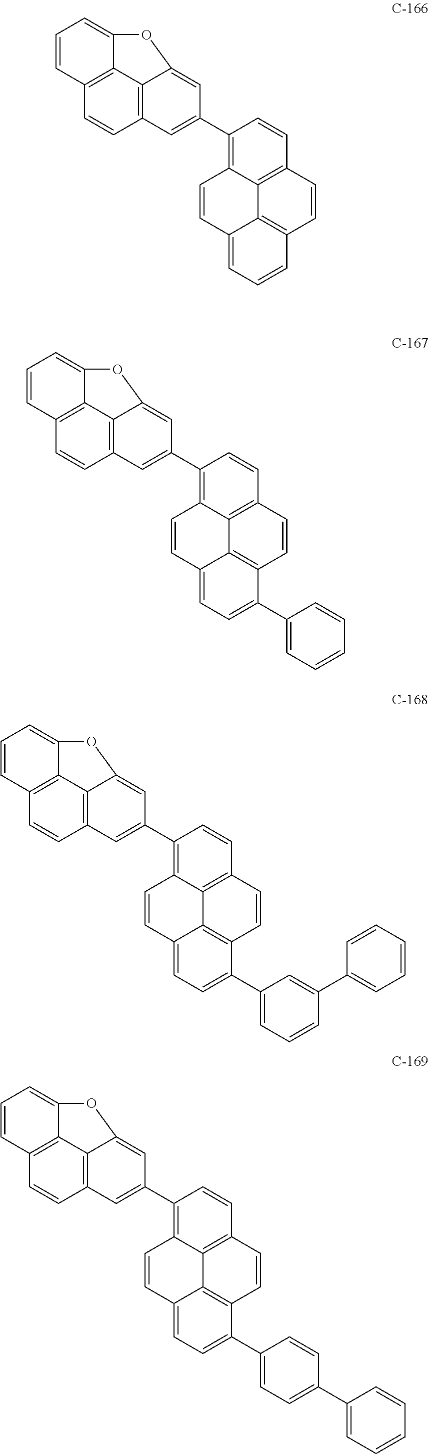 [SMILES: c1cc2ccc3ccc(-c4cc5ccc6cccc7oc(c4)c5c67)c4ccc(c1)c2c34 ; c1ccc(-c2ccc3ccc4c(-c5cc6ccc7cccc8oc(c5)c6c78)ccc5ccc2c3c54)cc1 ; c1ccc(-c2cccc(-c3ccc4ccc5c(-c6cc7ccc8cccc9oc(c6)c7c89)ccc6ccc3c4c65)c2)cc1 ; c1ccc(-c2ccc(-c3ccc4ccc5c(-c6cc7ccc8cccc9oc(c6)c7c89)ccc6ccc3c4c65)cc2)cc1]

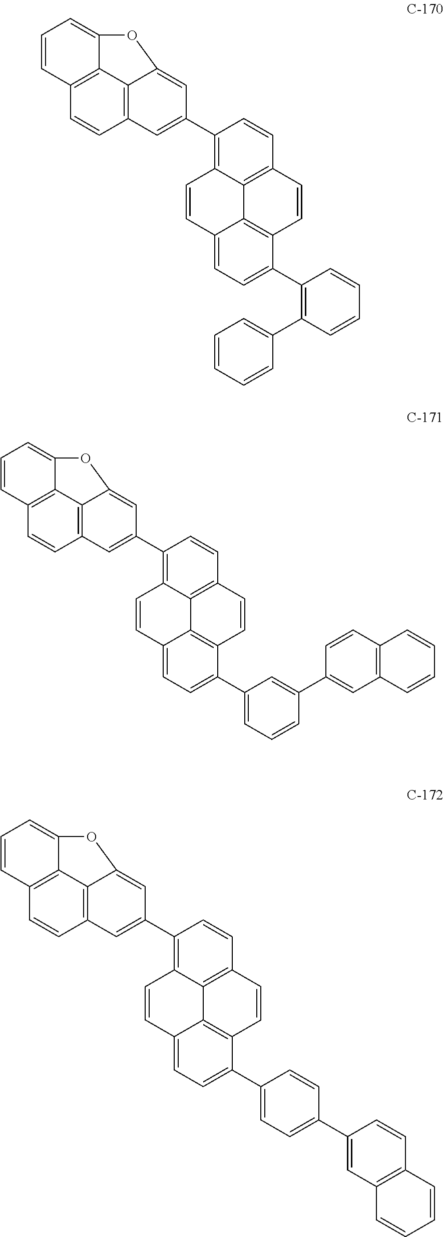 [SMILES: c1ccc(-c2ccccc2-c2ccc3ccc4c(-c5cc6ccc7cccc8oc(c5)c6c78)ccc5ccc2c3c54)cc1 ; c1cc(-c2ccc3ccccc3c2)cc(-c2ccc3ccc4c(-c5cc6ccc7cccc8oc(c5)c6c78)ccc5ccc2c3c54)c1 ; c1ccc2cc(-c3ccc(-c4ccc5ccc6c(-c7cc8ccc9cccc%10oc(c7)c8c9%10)ccc7ccc4c5c76)cc3)ccc2c1]

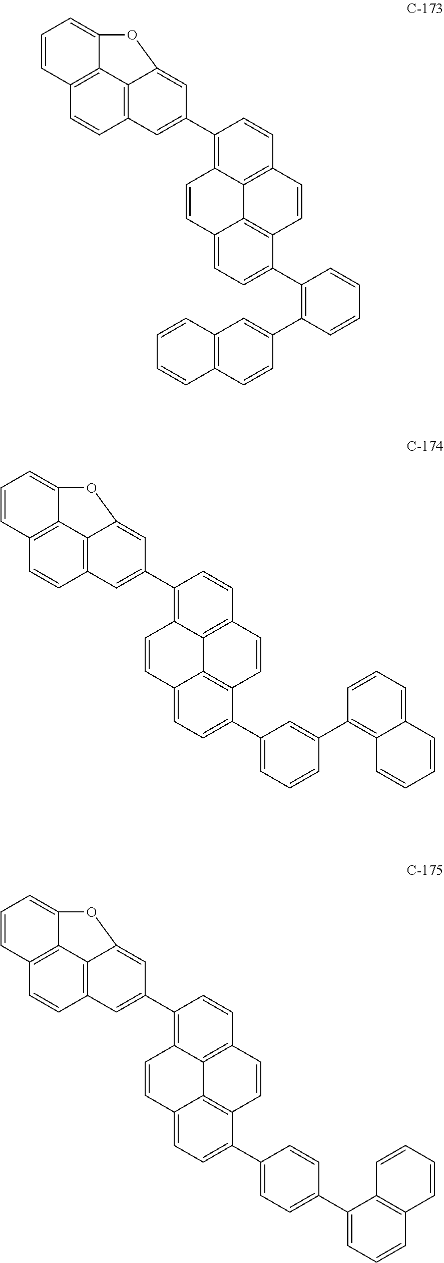 [SMILES: c1ccc(-c2ccc3ccc4c(-c5cc6ccc7cccc8oc(c5)c6c78)ccc5ccc2c3c54)c(-c2ccc3ccccc3c2)c1 ; c1cc(-c2cccc3ccccc23)cc(-c2ccc3ccc4c(-c5cc6ccc7cccc8oc(c5)c6c78)ccc5ccc2c3c54)c1 ; c1ccc2c(-c3ccc(-c4ccc5ccc6c(-c7cc8ccc9cccc%10oc(c7)c8c9%10)ccc7ccc4c5c76)cc3)cccc2c1]

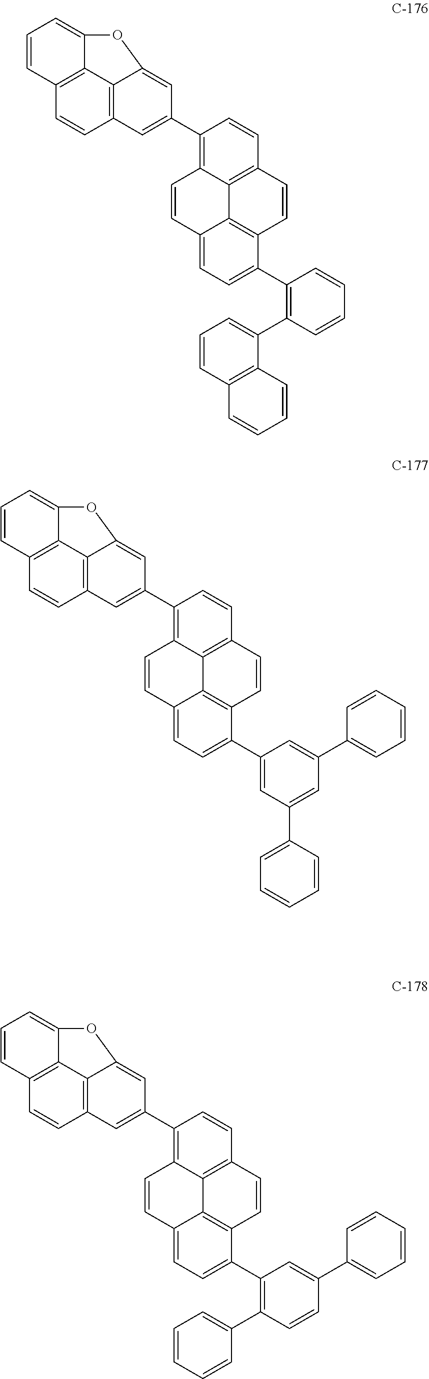 [SMILES: c1ccc(-c2ccc3ccc4c(-c5cc6ccc7cccc8oc(c5)c6c78)ccc5ccc2c3c54)c(-c2cccc3ccccc23)c1 ; c1ccc(-c2cc(-c3ccccc3)cc(-c3ccc4ccc5c(-c6cc7ccc8cccc9oc(c6)c7c89)ccc6ccc3c4c65)c2)cc1 ; c1ccc(-c2ccc(-c3ccccc3)c(-c3ccc4ccc5c(-c6cc7ccc8cccc9oc(c6)c7c89)ccc6ccc3c4c65)c2)cc1]

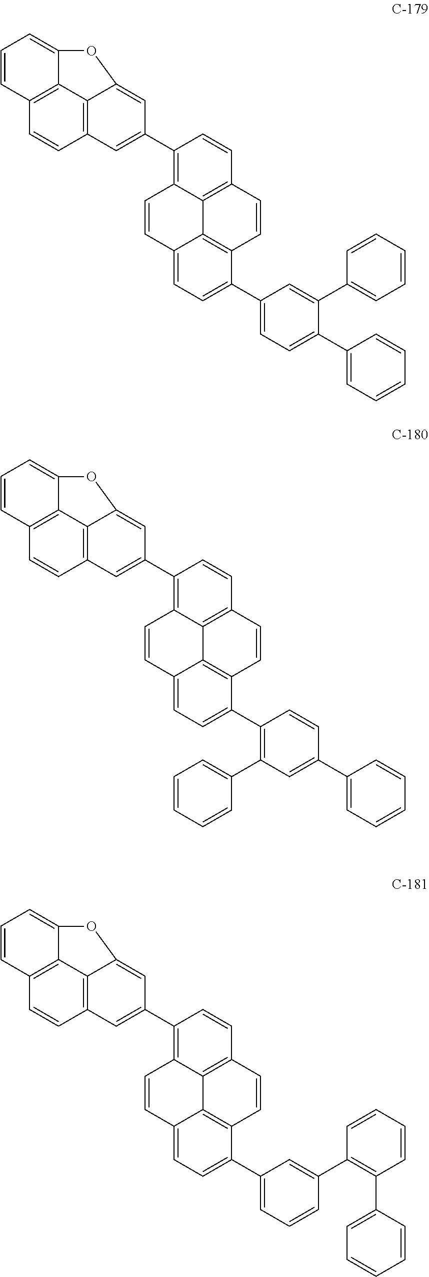 [SMILES: c1ccc(-c2ccc(-c3ccc4ccc5c(-c6cc7ccc8cccc9oc(c6)c7c89)ccc6ccc3c4c65)cc2-c2ccccc2)cc1 ; c1ccc(-c2ccc(-c3ccc4ccc5c(-c6cc7ccc8cccc9oc(c6)c7c89)ccc6ccc3c4c65)c(-c3ccccc3)c2)cc1 ; c1ccc(-c2ccccc2-c2cccc(-c3ccc4ccc5c(-c6cc7ccc8cccc9oc(c6)c7c89)ccc6ccc3c4c65)c2)cc1]

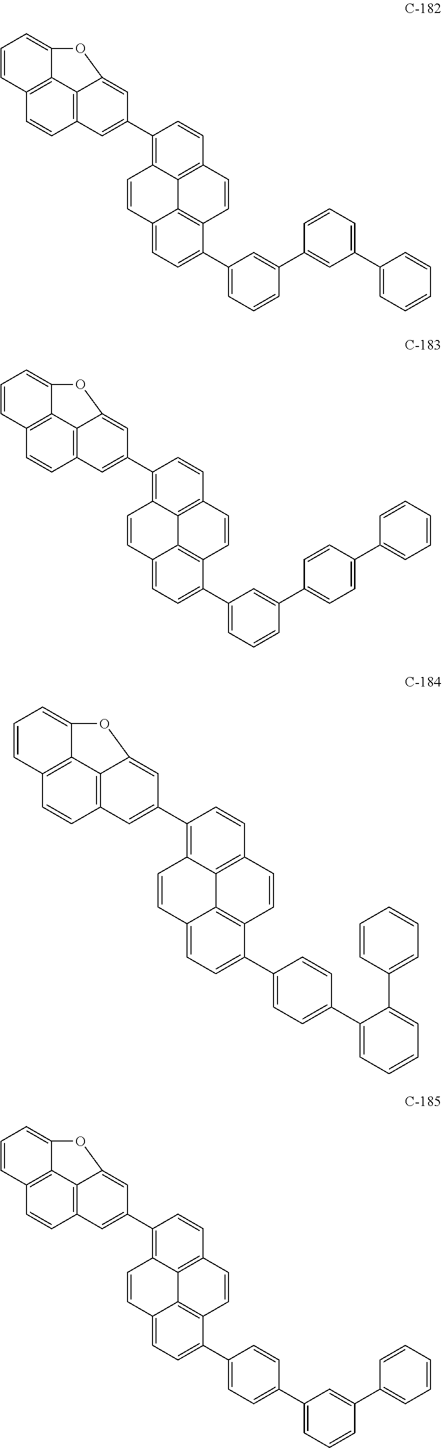 [SMILES: c1ccc(-c2cccc(-c3cccc(-c4ccc5ccc6c(-c7cc8ccc9cccc%10oc(c7)c8c9%10)ccc7ccc4c5c76)c3)c2)cc1 ; c1ccc(-c2ccc(-c3cccc(-c4ccc5ccc6c(-c7cc8ccc9cccc%10oc(c7)c8c9%10)ccc7ccc4c5c76)c3)cc2)cc1 ; c1ccc(-c2ccccc2-c2ccc(-c3ccc4ccc5c(-c6cc7ccc8cccc9oc(c6)c7c89)ccc6ccc3c4c65)cc2)cc1 ; c1ccc(-c2cccc(-c3ccc(-c4ccc5ccc6c(-c7cc8ccc9cccc%10oc(c7)c8c9%10)ccc7ccc4c5c76)cc3)c2)cc1]

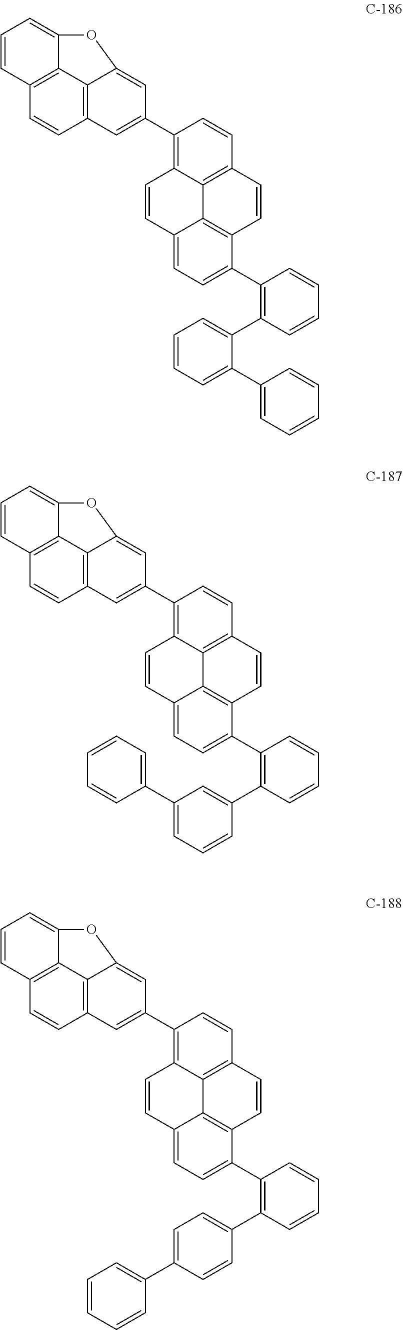 [SMILES: c1ccc(-c2ccccc2-c2ccccc2-c2ccc3ccc4c(-c5cc6ccc7cccc8oc(c5)c6c78)ccc5ccc2c3c54)cc1 ; c1ccc(-c2cccc(-c3ccccc3-c3ccc4ccc5c(-c6cc7ccc8cccc9oc(c6)c7c89)ccc6ccc3c4c65)c2)cc1 ; c1ccc(-c2ccc(-c3ccccc3-c3ccc4ccc5c(-c6cc7ccc8cccc9oc(c6)c7c89)ccc6ccc3c4c65)cc2)cc1]

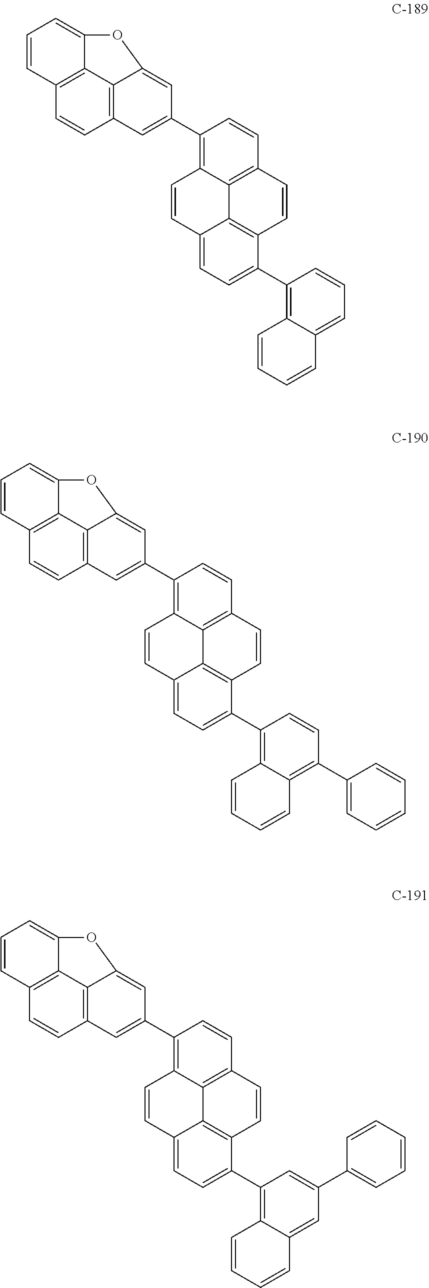 [SMILES: c1ccc2c(-c3ccc4ccc5c(-c6cc7ccc8cccc9oc(c6)c7c89)ccc6ccc3c4c65)cccc2c1 ; c1ccc(-c2ccc(-c3ccc4ccc5c(-c6cc7ccc8cccc9oc(c6)c7c89)ccc6ccc3c4c65)c3ccccc23)cc1 ; c1ccc(-c2cc(-c3ccc4ccc5c(-c6cc7ccc8cccc9oc(c6)c7c89)ccc6ccc3c4c65)c3ccccc3c2)cc1]

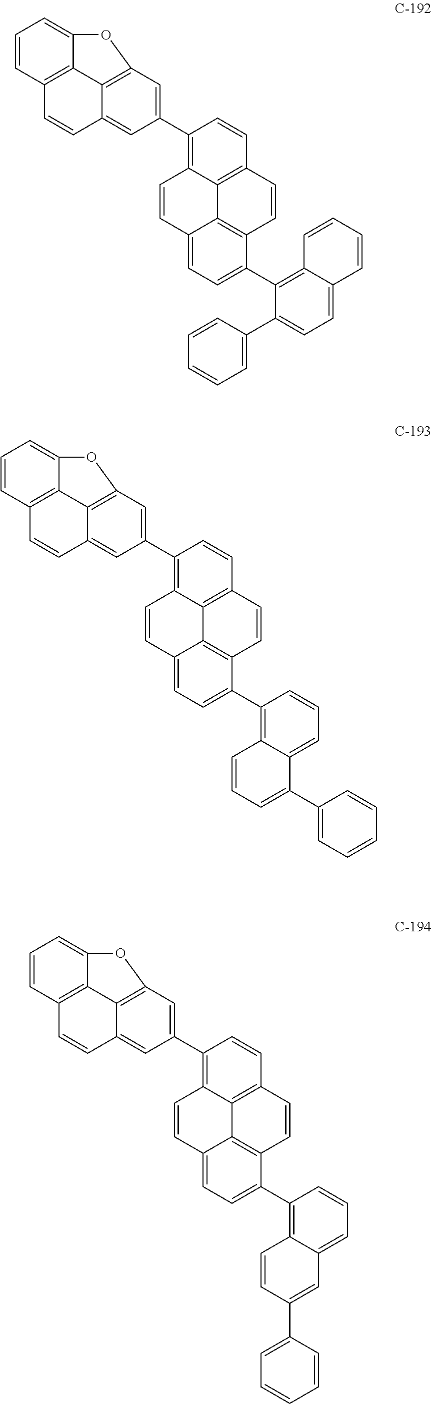 [SMILES: c1ccc(-c2ccc3ccccc3c2-c2ccc3ccc4c(-c5cc6ccc7cccc8oc(c5)c6c78)ccc5ccc2c3c54)cc1 ; c1ccc(-c2cccc3c(-c4ccc5ccc6c(-c7cc8ccc9cccc%10oc(c7)c8c9%10)ccc7ccc4c5c76)cccc23)cc1 ; c1ccc(-c2ccc3c(-c4ccc5ccc6c(-c7cc8ccc9cccc%10oc(c7)c8c9%10)ccc7ccc4c5c76)cccc3c2)cc1]

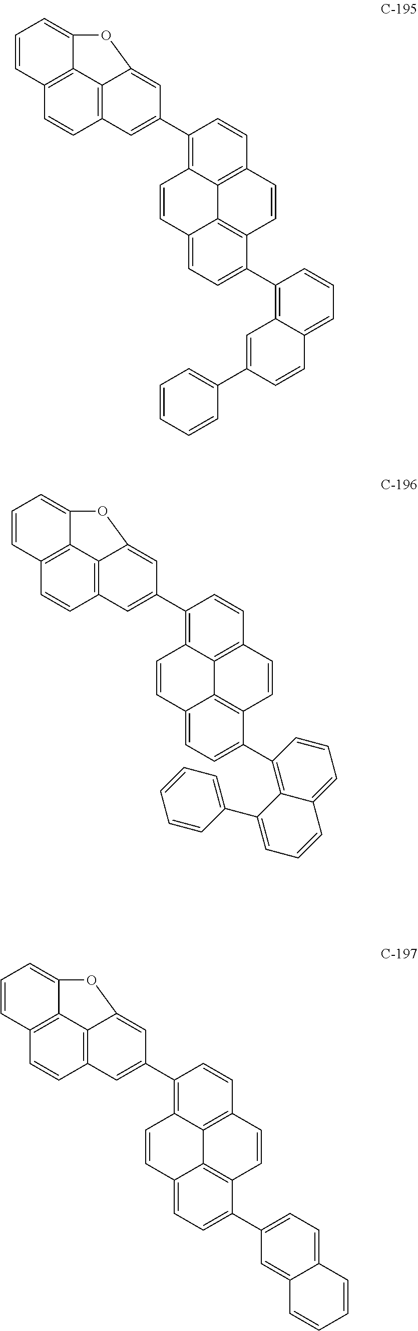 [SMILES: c1ccc(-c2ccc3cccc(-c4ccc5ccc6c(-c7cc8ccc9cccc%10oc(c7)c8c9%10)ccc7ccc4c5c76)c3c2)cc1 ; c1ccc(-c2cccc3cccc(-c4ccc5ccc6c(-c7cc8ccc9cccc%10oc(c7)c8c9%10)ccc7ccc4c5c76)c23)cc1 ; c1ccc2cc(-c3ccc4ccc5c(-c6cc7ccc8cccc9oc(c6)c7c89)ccc6ccc3c4c65)ccc2c1]

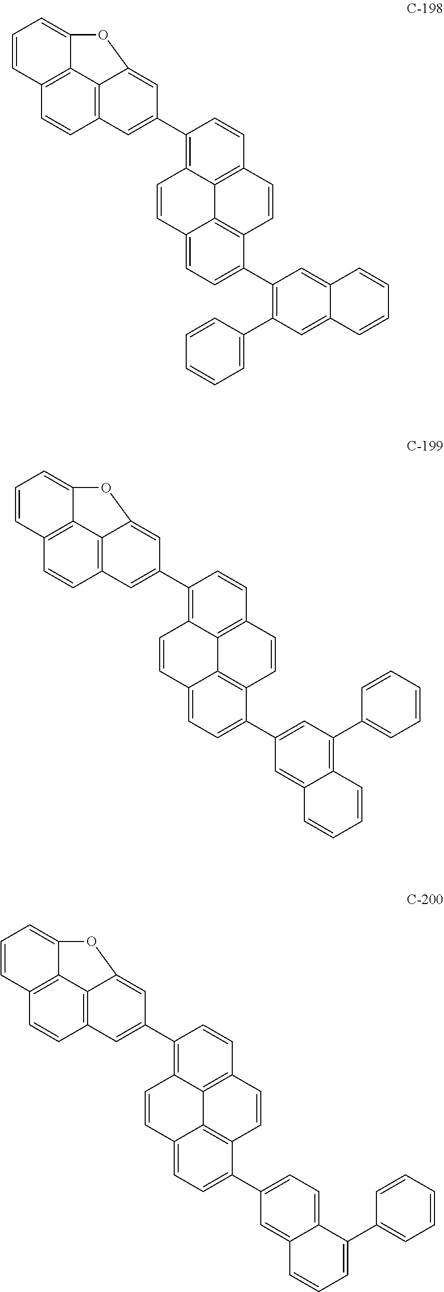 [SMILES: c1ccc(-c2cc3ccccc3cc2-c2ccc3ccc4c(-c5cc6ccc7cccc8oc(c5)c6c78)ccc5ccc2c3c54)cc1 ; c1ccc(-c2cc(-c3ccc4ccc5c(-c6cc7ccc8cccc9oc(c6)c7c89)ccc6ccc3c4c65)cc3ccccc23)cc1 ; c1ccc(-c2cccc3cc(-c4ccc5ccc6c(-c7cc8ccc9cccc%10oc(c7)c8c9%10)ccc7ccc4c5c76)ccc23)cc1]

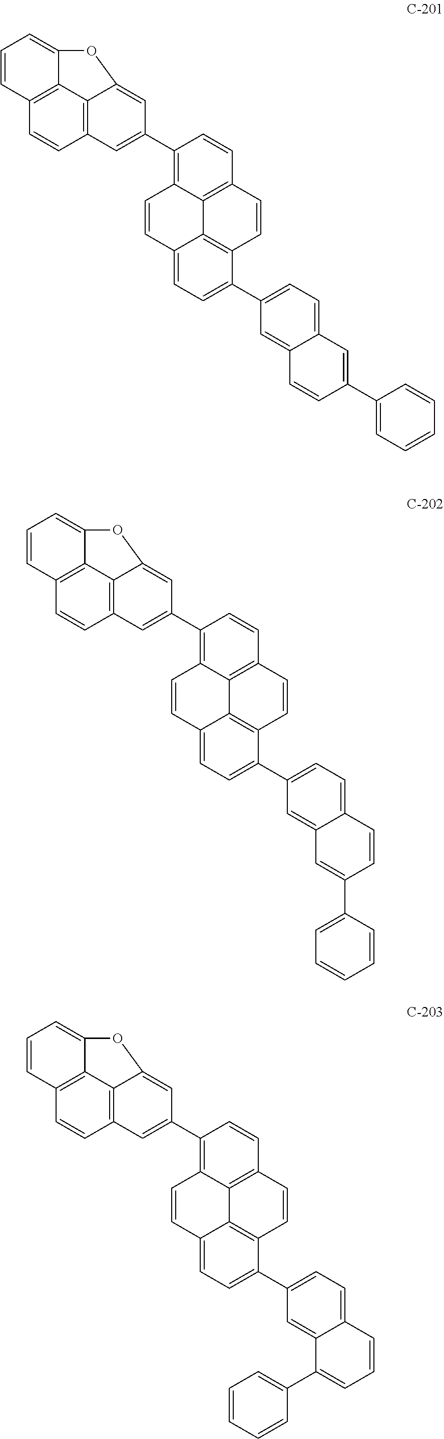 [SMILES: c1ccc(-c2ccc3cc(-c4ccc5ccc6c(-c7cc8ccc9cccc%10oc(c7)c8c9%10)ccc7ccc4c5c76)ccc3c2)cc1 ; c1ccc(-c2ccc3ccc(-c4ccc5ccc6c(-c7cc8ccc9cccc%10oc(c7)c8c9%10)ccc7ccc4c5c76)cc3c2)cc1 ; c1ccc(-c2cccc3ccc(-c4ccc5ccc6c(-c7cc8ccc9cccc%10oc(c7)c8c9%10)ccc7ccc4c5c76)cc23)cc1]

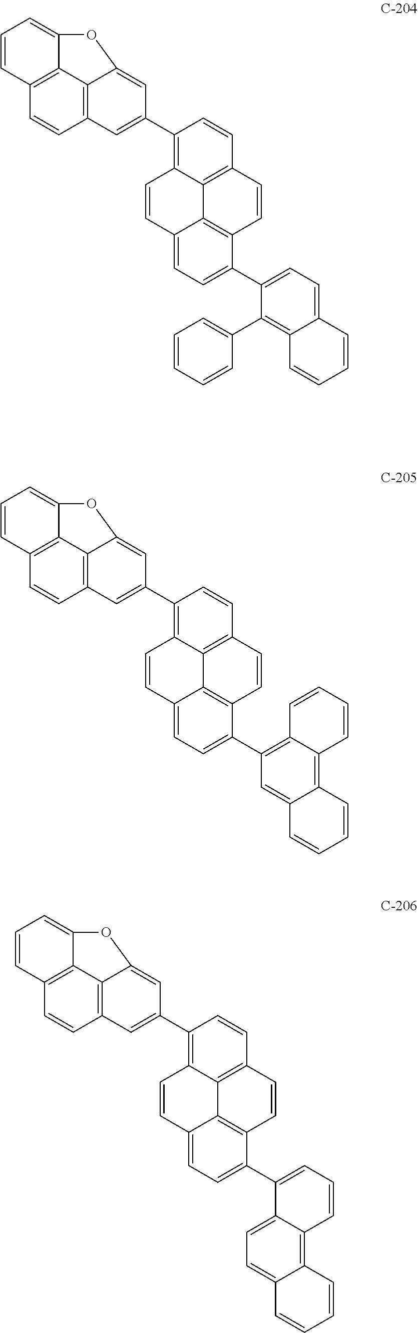 [SMILES: c1ccc(-c2c(-c3ccc4ccc5c(-c6cc7ccc8cccc9oc(c6)c7c89)ccc6ccc3c4c65)ccc3ccccc23)cc1 ; c1ccc2c(c1)cc(-c1ccc3ccc4c(-c5cc6ccc7cccc8oc(c5)c6c78)ccc5ccc1c3c54)c1ccccc12 ; c1ccc2c(c1)ccc1c(-c3ccc4ccc5c(-c6cc7ccc8cccc9oc(c6)c7c89)ccc6ccc3c4c65)cccc12]

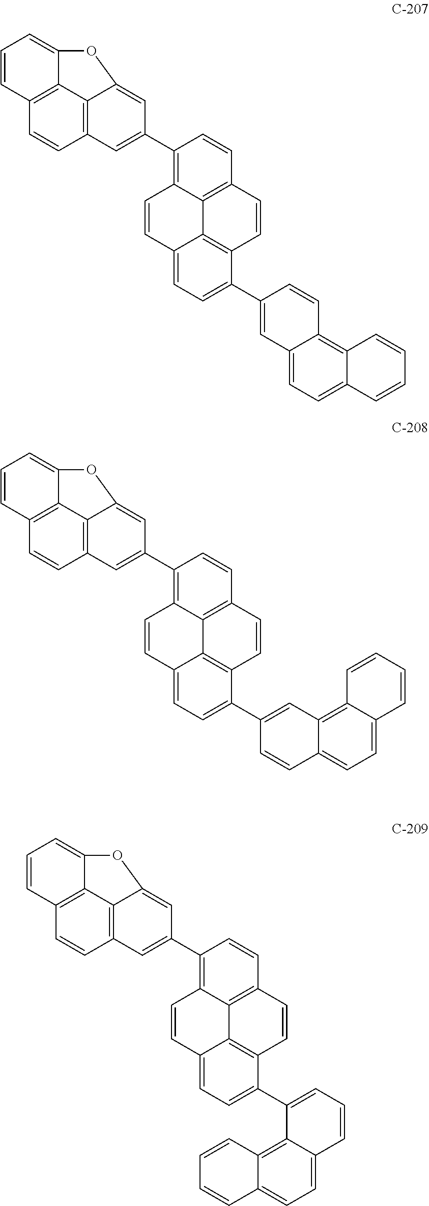 [SMILES: c1ccc2c(c1)ccc1cc(-c3ccc4ccc5c(-c6cc7ccc8cccc9oc(c6)c7c89)ccc6ccc3c4c65)ccc12 ; c1ccc2c(c1)ccc1ccc(-c3ccc4ccc5c(-c6cc7ccc8cccc9oc(c6)c7c89)ccc6ccc3c4c65)cc12 ; c1ccc2c(c1)ccc1cccc(-c3ccc4ccc5c(-c6cc7ccc8cccc9oc(c6)c7c89)ccc6ccc3c4c65)c12]

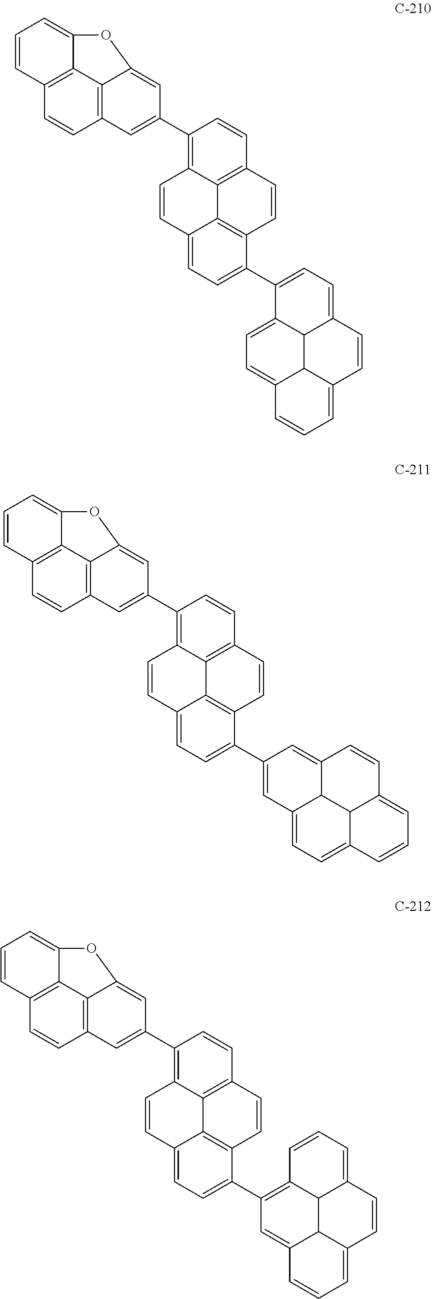 [SMILES: C1=CC2=CC=C3C(c4ccc5ccc6c(-c7cc8ccc9cccc%10oc(c7)c8c9%10)ccc7ccc4c5c76)=CC=C4C=CC(=C1)C2C43 ; C1=CC2=CC=C3C=C(c4ccc5ccc6c(-c7cc8ccc9cccc%10oc(c7)c8c9%10)ccc7ccc4c5c76)C=C4C=CC(=C1)C2C34 ; C1=CC2=CC(c3ccc4ccc5c(-c6cc7ccc8cccc9oc(c6)c7c89)ccc6ccc3c4c65)=C3C=CC=C4C=CC(=C1)C2C43]

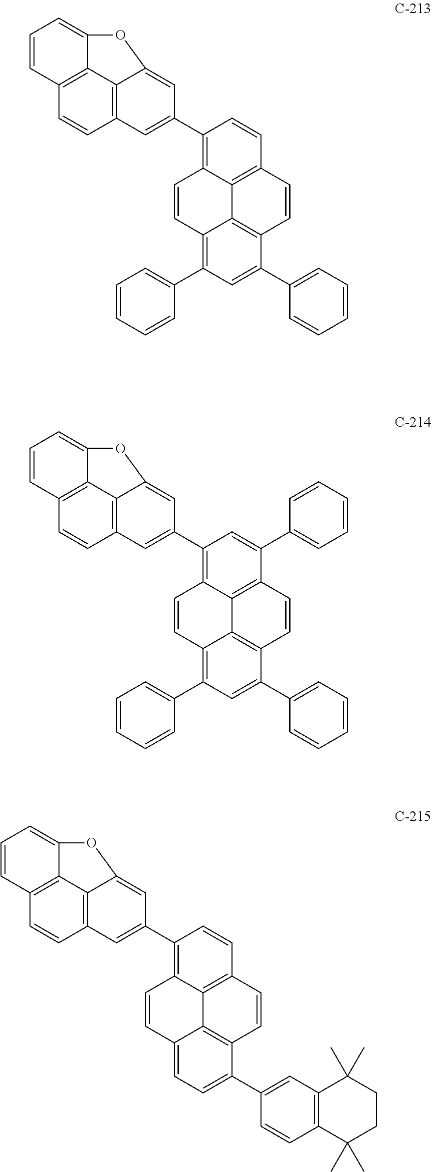 [SMILES: c1ccc(-c2cc(-c3ccccc3)c3ccc4c(-c5cc6ccc7cccc8oc(c5)c6c78)ccc5ccc2c3c54)cc1 ; c1ccc(-c2cc(-c3ccccc3)c3ccc4c(-c5cc6ccc7cccc8oc(c5)c6c78)cc(-c5ccccc5)c5ccc2c3c54)cc1 ; CC1(C)CCC(C)(C)c2cc(-c3ccc4ccc5c(-c6cc7ccc8cccc9oc(c6)c7c89)ccc6ccc3c4c65)ccc21]

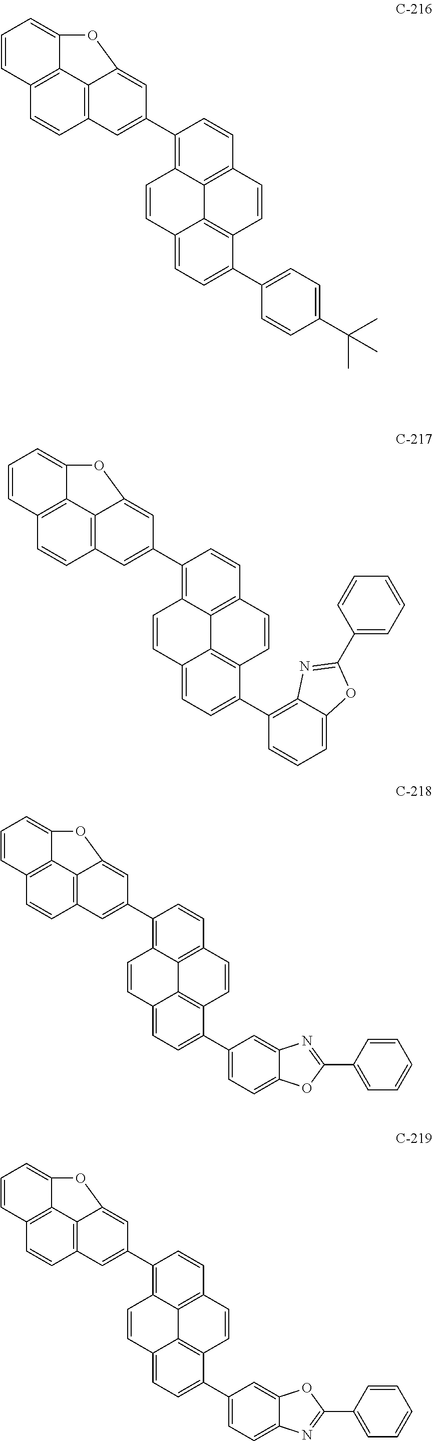 [SMILES: CC(C)(C)c1ccc(-c2ccc3ccc4c(-c5cc6ccc7cccc8oc(c5)c6c78)ccc5ccc2c3c54)cc1 ; c1ccc(-c2nc3c(-c4ccc5ccc6c(-c7cc8ccc9cccc%10oc(c7)c8c9%10)ccc7ccc4c5c76)cccc3o2)cc1 ; c1ccc(-c2nc3cc(-c4ccc5ccc6c(-c7cc8ccc9cccc%10oc(c7)c8c9%10)ccc7ccc4c5c76)ccc3o2)cc1 ; c1ccc(-c2nc3ccc(-c4ccc5ccc6c(-c7cc8ccc9cccc%10oc(c7)c8c9%10)ccc7ccc4c5c76)cc3o2)cc1]

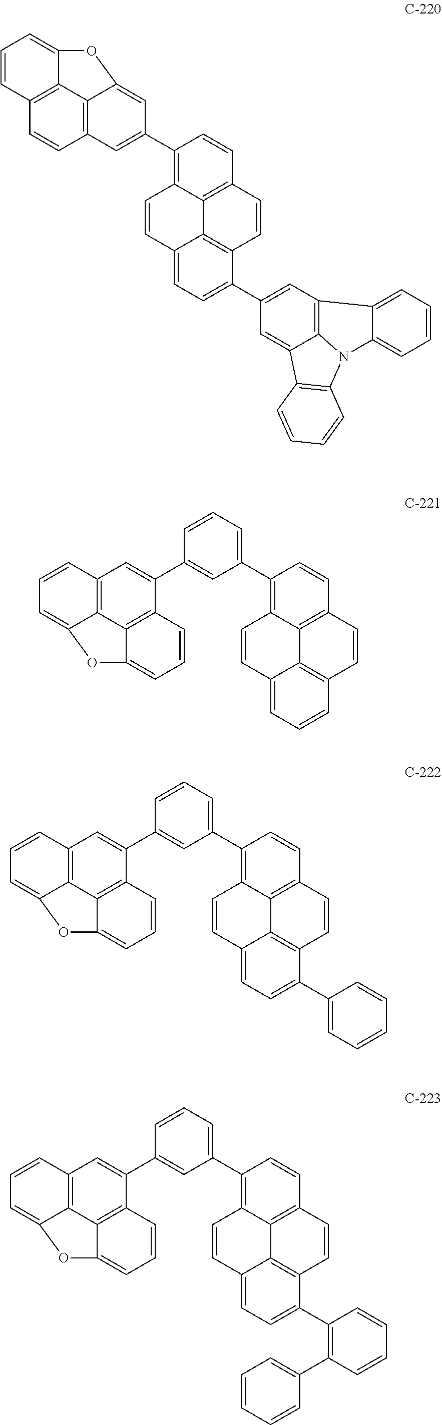 [SMILES: c1cc2ccc3cc(-c4ccc5ccc6c(-c7cc8c9ccccc9n9c%10ccccc%10c(c7)c89)ccc7ccc4c5c76)cc4oc(c1)c2c34 ; c1cc(-c2ccc3ccc4cccc5ccc2c3c45)cc(-c2cc3cccc4oc5cccc2c5c34)c1 ; c1ccc(-c2ccc3ccc4c(-c5cccc(-c6cc7cccc8oc9cccc6c9c78)c5)ccc5ccc2c3c54)cc1 ; c1ccc(-c2ccccc2-c2ccc3ccc4c(-c5cccc(-c6cc7cccc8oc9cccc6c9c78)c5)ccc5ccc2c3c54)cc1]

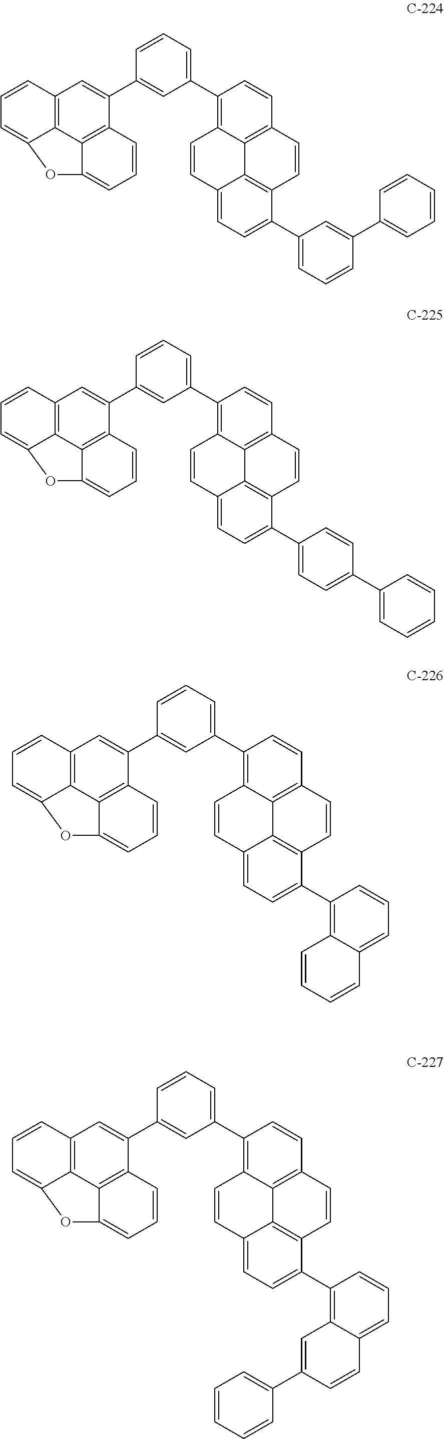 [SMILES: c1ccc(-c2cccc(-c3ccc4ccc5c(-c6cccc(-c7cc8cccc9oc%10cccc7c%10c89)c6)ccc6ccc3c4c65)c2)cc1 ; c1ccc(-c2ccc(-c3ccc4ccc5c(-c6cccc(-c7cc8cccc9oc%10cccc7c%10c89)c6)ccc6ccc3c4c65)cc2)cc1 ; c1cc(-c2ccc3ccc4c(-c5cccc6ccccc56)ccc5ccc2c3c54)cc(-c2cc3cccc4oc5cccc2c5c34)c1 ; c1ccc(-c2ccc3cccc(-c4ccc5ccc6c(-c7cccc(-c8cc9cccc%10oc%11cccc8c%11c9%10)c7)ccc7ccc4c5c76)c3c2)cc1]

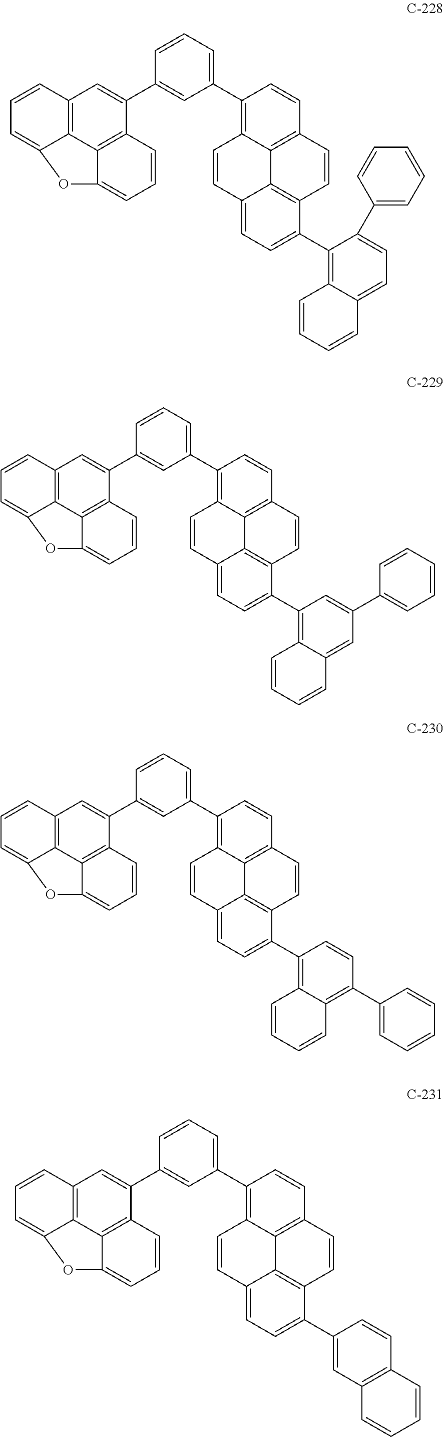 [SMILES: c1ccc(-c2ccc3ccccc3c2-c2ccc3ccc4c(-c5cccc(-c6cc7cccc8oc9cccc6c9c78)c5)ccc5ccc2c3c54)cc1 ; c1ccc(-c2cc(-c3ccc4ccc5c(-c6cccc(-c7cc8cccc9oc%10cccc7c%10c89)c6)ccc6ccc3c4c65)c3ccccc3c2)cc1 ; c1ccc(-c2ccc(-c3ccc4ccc5c(-c6cccc(-c7cc8cccc9oc%10cccc7c%10c89)c6)ccc6ccc3c4c65)c3ccccc23)cc1 ; c1cc(-c2ccc3ccc4c(-c5ccc6ccccc6c5)ccc5ccc2c3c54)cc(-c2cc3cccc4oc5cccc2c5c34)c1]

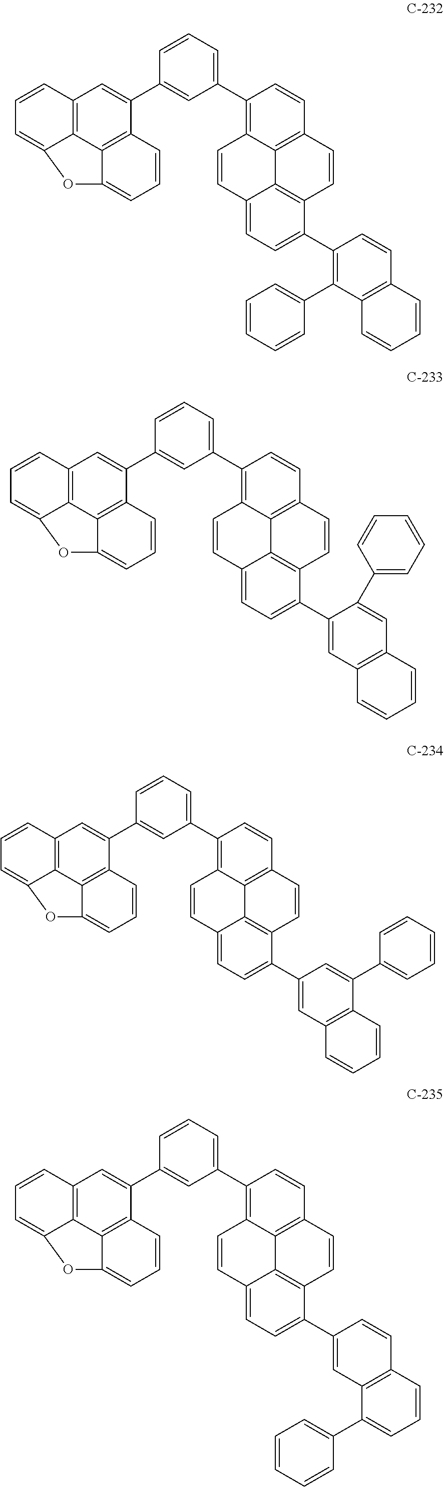 [SMILES: c1ccc(-c2c(-c3ccc4ccc5c(-c6cccc(-c7cc8cccc9oc%10cccc7c%10c89)c6)ccc6ccc3c4c65)ccc3ccccc23)cc1 ; c1ccc(-c2cc3ccccc3cc2-c2ccc3ccc4c(-c5cccc(-c6cc7cccc8oc9cccc6c9c78)c5)ccc5ccc2c3c54)cc1 ; c1ccc(-c2cc(-c3ccc4ccc5c(-c6cccc(-c7cc8cccc9oc%10cccc7c%10c89)c6)ccc6ccc3c4c65)cc3ccccc23)cc1 ; c1ccc(-c2cccc3ccc(-c4ccc5ccc6c(-c7cccc(-c8cc9cccc%10oc%11cccc8c%11c9%10)c7)ccc7ccc4c5c76)cc23)cc1]

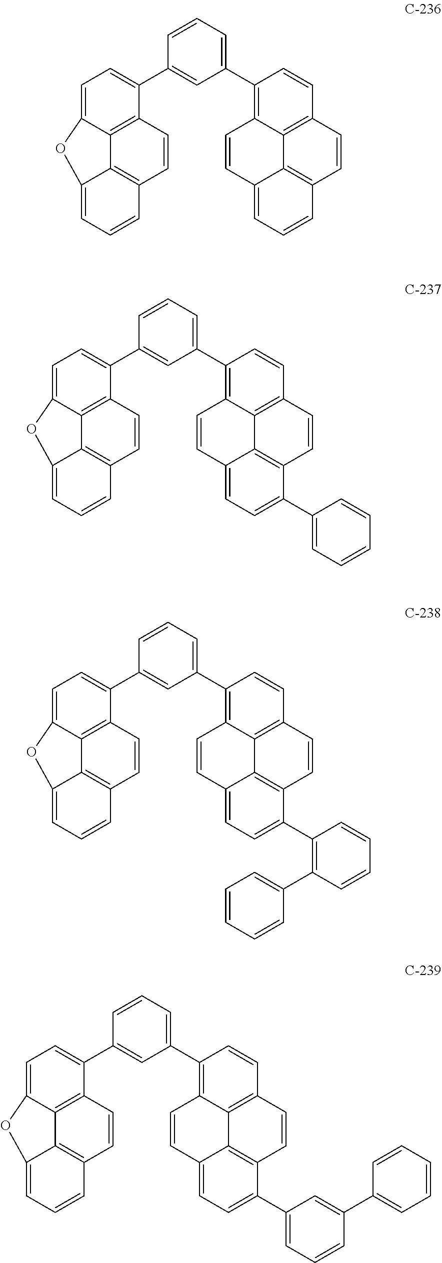 [SMILES: c1cc(-c2ccc3ccc4cccc5ccc2c3c45)cc(-c2ccc3oc4cccc5ccc2c3c54)c1 ; c1ccc(-c2ccc3ccc4c(-c5cccc(-c6ccc7oc8cccc9ccc6c7c98)c5)ccc5ccc2c3c54)cc1 ; c1ccc(-c2ccccc2-c2ccc3ccc4c(-c5cccc(-c6ccc7oc8cccc9ccc6c7c98)c5)ccc5ccc2c3c54)cc1 ; c1ccc(-c2cccc(-c3ccc4ccc5c(-c6cccc(-c7ccc8oc9cccc%10ccc7c8c%109)c6)ccc6ccc3c4c65)c2)cc1]

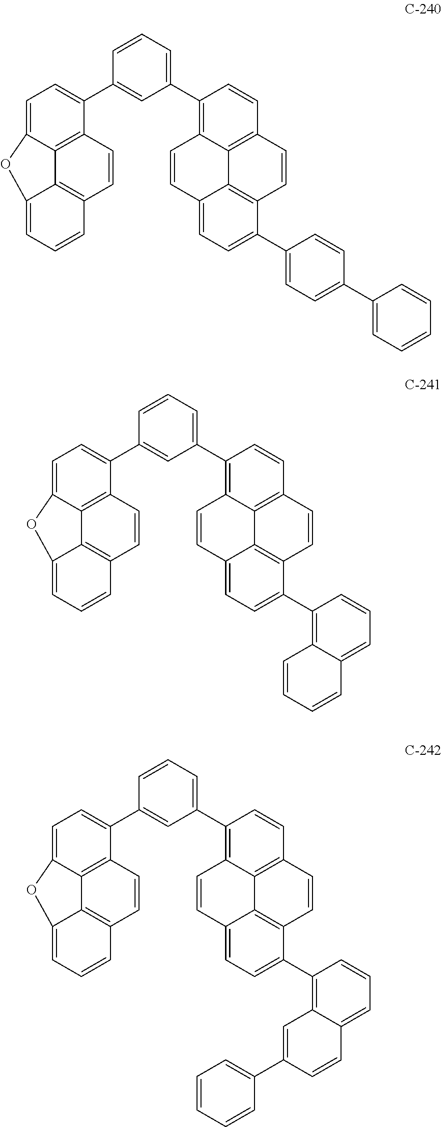 [SMILES: c1ccc(-c2ccc(-c3ccc4ccc5c(-c6cccc(-c7ccc8oc9cccc%10ccc7c8c%109)c6)ccc6ccc3c4c65)cc2)cc1 ; c1cc(-c2ccc3ccc4c(-c5cccc6ccccc56)ccc5ccc2c3c54)cc(-c2ccc3oc4cccc5ccc2c3c54)c1 ; c1ccc(-c2ccc3cccc(-c4ccc5ccc6c(-c7cccc(-c8ccc9oc%10cccc%11ccc8c9c%11%10)c7)ccc7ccc4c5c76)c3c2)cc1]

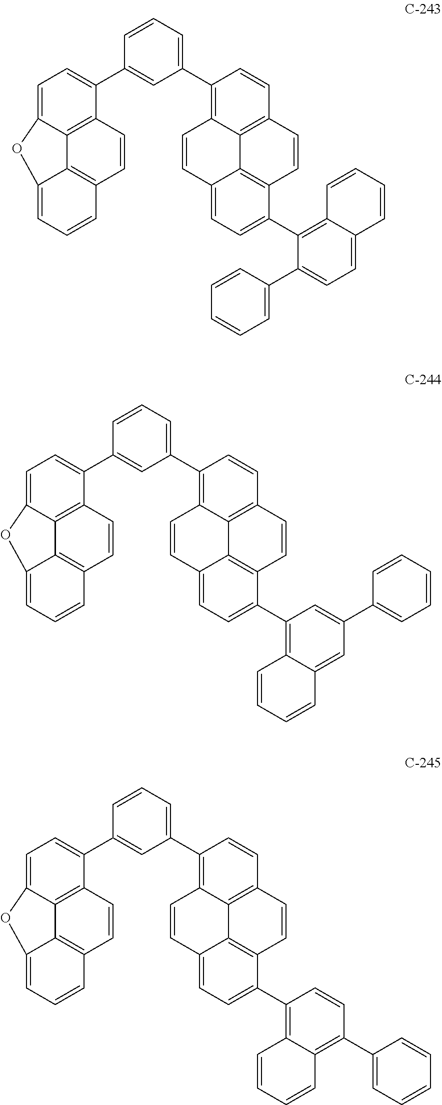 [SMILES: c1ccc(-c2ccc3ccccc3c2-c2ccc3ccc4c(-c5cccc(-c6ccc7oc8cccc9ccc6c7c98)c5)ccc5ccc2c3c54)cc1 ; c1ccc(-c2cc(-c3ccc4ccc5c(-c6cccc(-c7ccc8oc9cccc%10ccc7c8c%109)c6)ccc6ccc3c4c65)c3ccccc3c2)cc1 ; c1ccc(-c2ccc(-c3ccc4ccc5c(-c6cccc(-c7ccc8oc9cccc%10ccc7c8c%109)c6)ccc6ccc3c4c65)c3ccccc23)cc1]

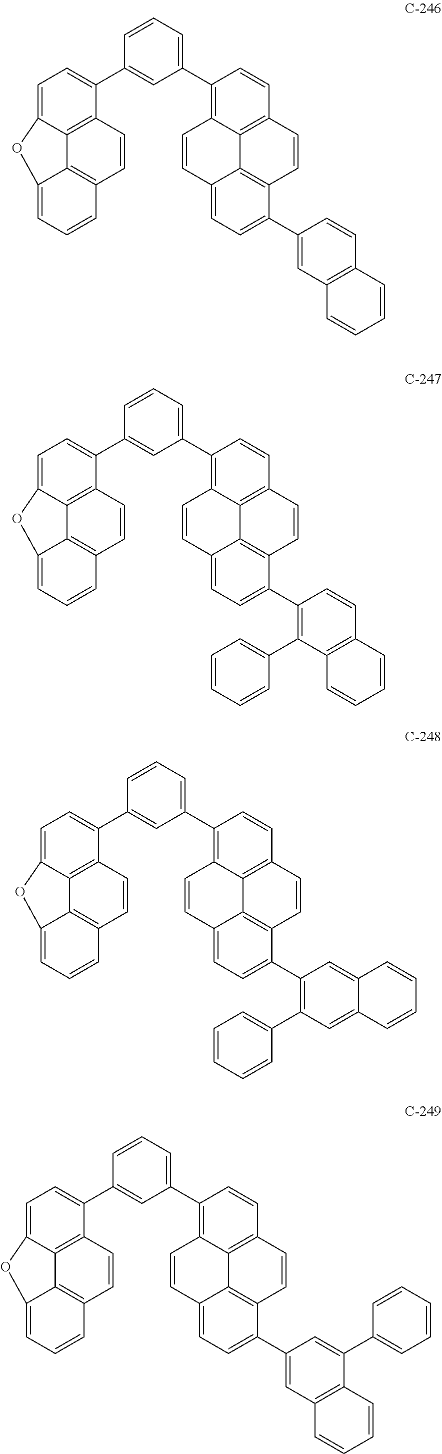 [SMILES: c1cc(-c2ccc3ccc4c(-c5ccc6ccccc6c5)ccc5ccc2c3c54)cc(-c2ccc3oc4cccc5ccc2c3c54)c1 ; c1ccc(-c2c(-c3ccc4ccc5c(-c6cccc(-c7ccc8oc9cccc%10ccc7c8c%109)c6)ccc6ccc3c4c65)ccc3ccccc23)cc1 ; c1ccc(-c2cc3ccccc3cc2-c2ccc3ccc4c(-c5cccc(-c6ccc7oc8cccc9ccc6c7c98)c5)ccc5ccc2c3c54)cc1 ; c1ccc(-c2cc(-c3ccc4ccc5c(-c6cccc(-c7ccc8oc9cccc%10ccc7c8c%109)c6)ccc6ccc3c4c65)cc3ccccc23)cc1]

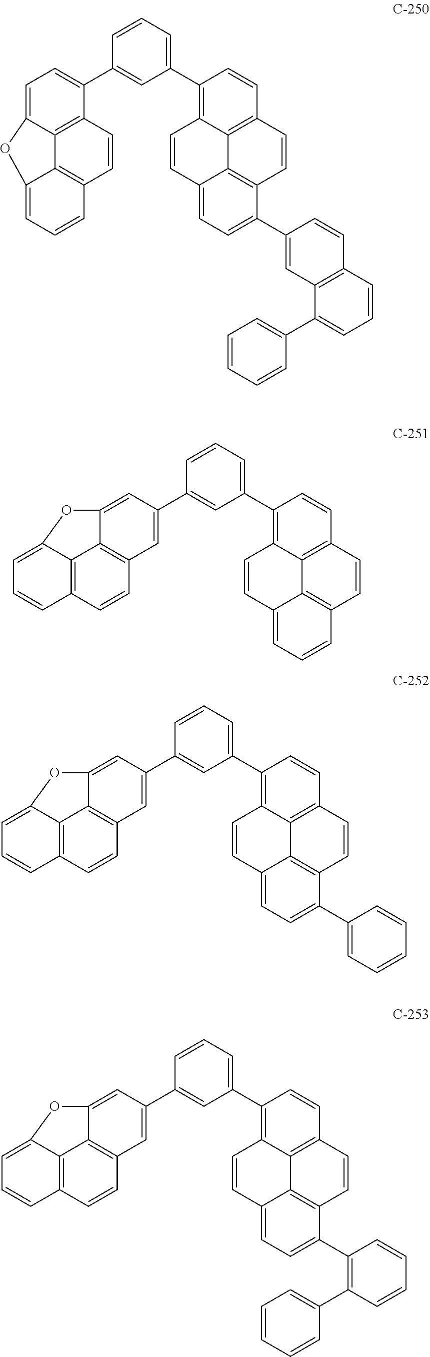 [SMILES: c1ccc(-c2cccc3ccc(-c4ccc5ccc6c(-c7cccc(-c8ccc9oc%10cccc%11ccc8c9c%11%10)c7)ccc7ccc4c5c76)cc23)cc1 ; c1cc(-c2cc3ccc4cccc5oc(c2)c3c45)cc(-c2ccc3ccc4cccc5ccc2c3c45)c1 ; c1ccc(-c2ccc3ccc4c(-c5cccc(-c6cc7ccc8cccc9oc(c6)c7c89)c5)ccc5ccc2c3c54)cc1 ; c1ccc(-c2ccccc2-c2ccc3ccc4c(-c5cccc(-c6cc7ccc8cccc9oc(c6)c7c89)c5)ccc5ccc2c3c54)cc1]

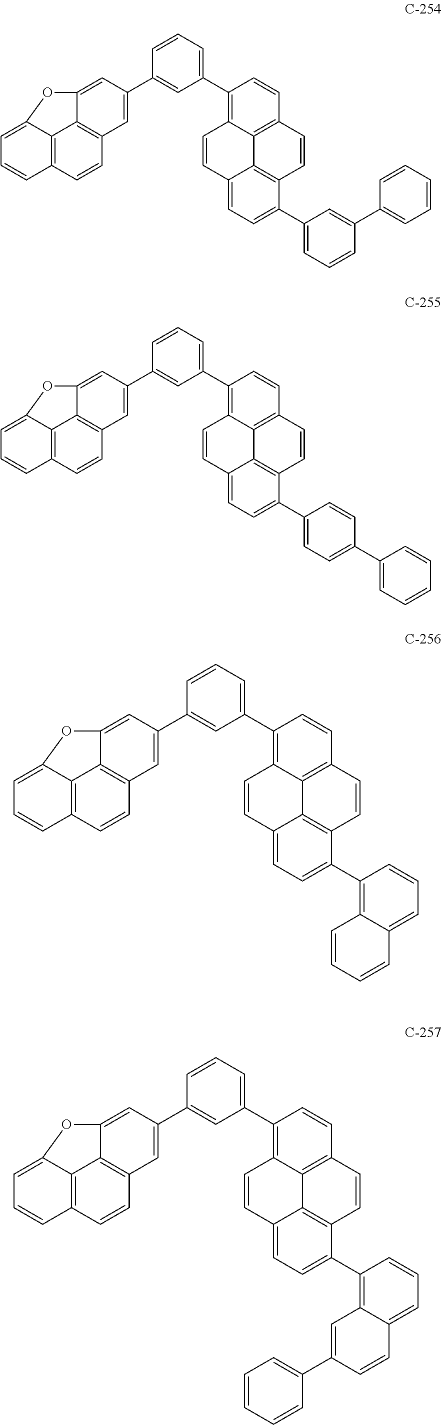 [SMILES: c1ccc(-c2cccc(-c3ccc4ccc5c(-c6cccc(-c7cc8ccc9cccc%10oc(c7)c8c9%10)c6)ccc6ccc3c4c65)c2)cc1 ; c1ccc(-c2ccc(-c3ccc4ccc5c(-c6cccc(-c7cc8ccc9cccc%10oc(c7)c8c9%10)c6)ccc6ccc3c4c65)cc2)cc1 ; c1cc(-c2cc3ccc4cccc5oc(c2)c3c45)cc(-c2ccc3ccc4c(-c5cccc6ccccc56)ccc5ccc2c3c54)c1 ; c1ccc(-c2ccc3cccc(-c4ccc5ccc6c(-c7cccc(-c8cc9ccc%10cccc%11oc(c8)c9c%10%11)c7)ccc7ccc4c5c76)c3c2)cc1]

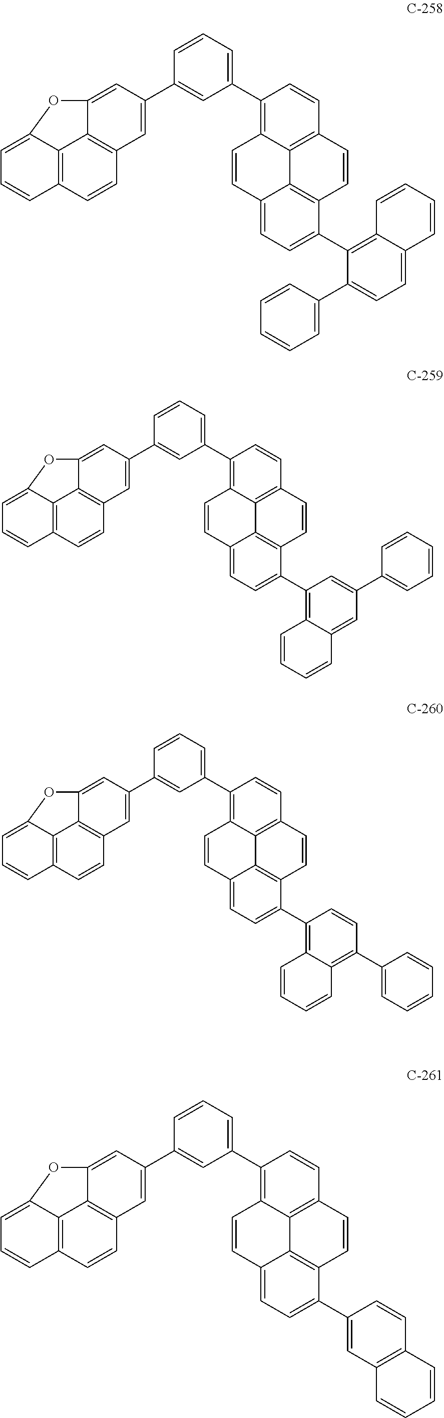 [SMILES: c1ccc(-c2ccc3ccccc3c2-c2ccc3ccc4c(-c5cccc(-c6cc7ccc8cccc9oc(c6)c7c89)c5)ccc5ccc2c3c54)cc1 ; c1ccc(-c2cc(-c3ccc4ccc5c(-c6cccc(-c7cc8ccc9cccc%10oc(c7)c8c9%10)c6)ccc6ccc3c4c65)c3ccccc3c2)cc1 ; c1ccc(-c2ccc(-c3ccc4ccc5c(-c6cccc(-c7cc8ccc9cccc%10oc(c7)c8c9%10)c6)ccc6ccc3c4c65)c3ccccc23)cc1 ; c1cc(-c2cc3ccc4cccc5oc(c2)c3c45)cc(-c2ccc3ccc4c(-c5ccc6ccccc6c5)ccc5ccc2c3c54)c1]

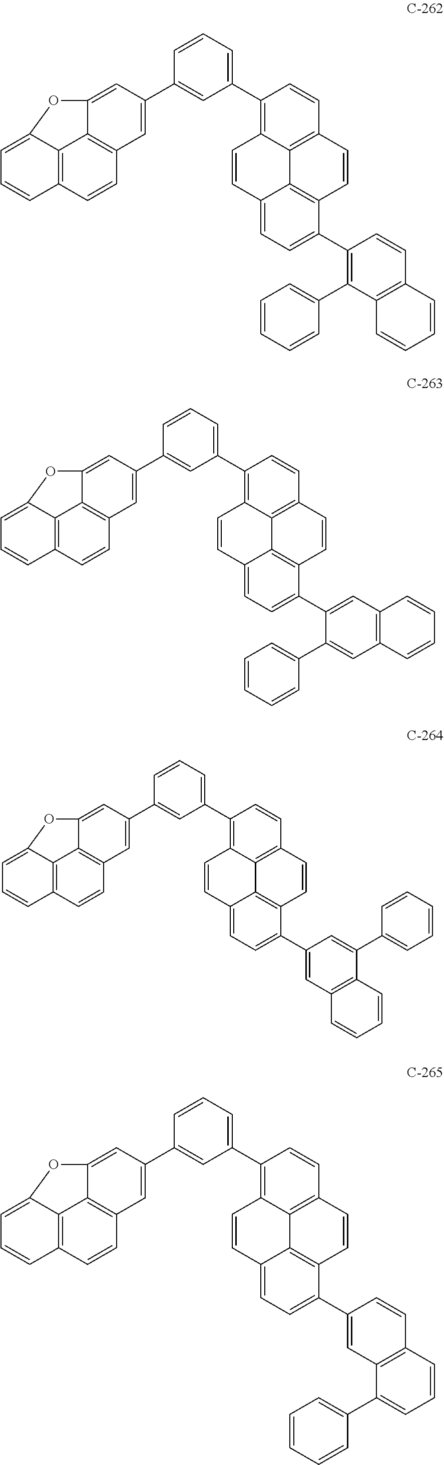 [SMILES: c1ccc(-c2c(-c3ccc4ccc5c(-c6cccc(-c7cc8ccc9cccc%10oc(c7)c8c9%10)c6)ccc6ccc3c4c65)ccc3ccccc23)cc1 ; c1ccc(-c2cc3ccccc3cc2-c2ccc3ccc4c(-c5cccc(-c6cc7ccc8cccc9oc(c6)c7c89)c5)ccc5ccc2c3c54)cc1 ; c1ccc(-c2cc(-c3ccc4ccc5c(-c6cccc(-c7cc8ccc9cccc%10oc(c7)c8c9%10)c6)ccc6ccc3c4c65)cc3ccccc23)cc1 ; c1ccc(-c2cccc3ccc(-c4ccc5ccc6c(-c7cccc(-c8cc9ccc%10cccc%11oc(c8)c9c%10%11)c7)ccc7ccc4c5c76)cc23)cc1]

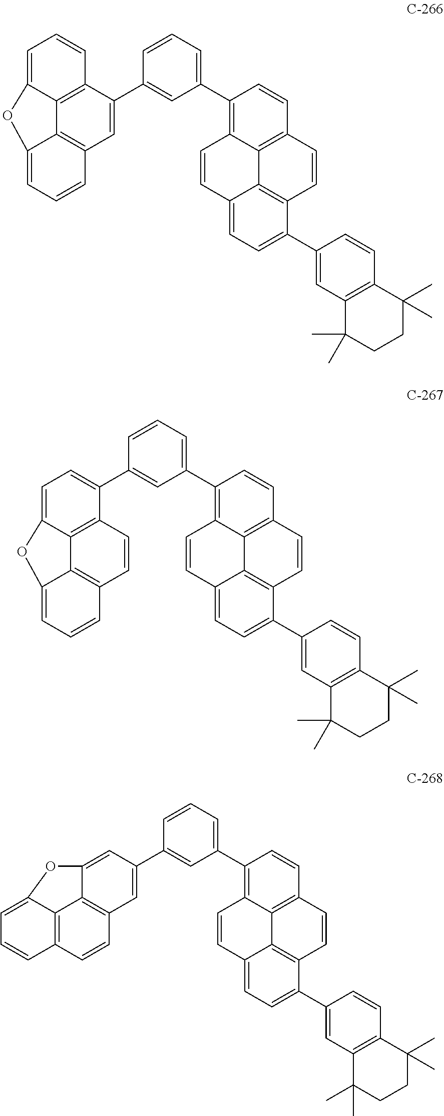 [SMILES: CC1(C)CCC(C)(C)c2cc(-c3ccc4ccc5c(-c6cccc(-c7cc8cccc9oc%10cccc7c%10c89)c6)ccc6ccc3c4c65)ccc21 ; CC1(C)CCC(C)(C)c2cc(-c3ccc4ccc5c(-c6cccc(-c7ccc8oc9cccc%10ccc7c8c%109)c6)ccc6ccc3c4c65)ccc21 ; CC1(C)CCC(C)(C)c2cc(-c3ccc4ccc5c(-c6cccc(-c7cc8ccc9cccc%10oc(c7)c8c9%10)c6)ccc6ccc3c4c65)ccc21]

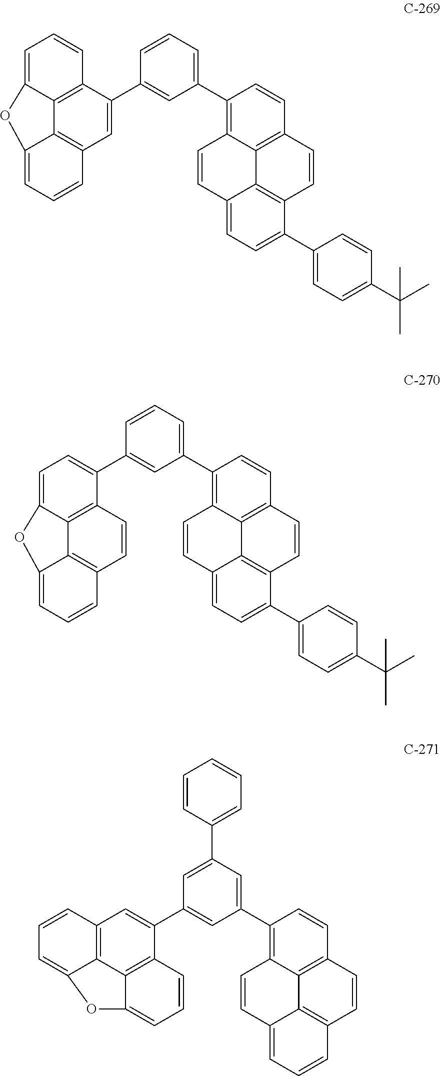 [SMILES: CC(C)(C)c1ccc(-c2ccc3ccc4c(-c5cccc(-c6cc7cccc8oc9cccc6c9c78)c5)ccc5ccc2c3c54)cc1 ; CC(C)(C)c1ccc(-c2ccc3ccc4c(-c5cccc(-c6ccc7oc8cccc9ccc6c7c98)c5)ccc5ccc2c3c54)cc1 ; c1ccc(-c2cc(-c3ccc4ccc5cccc6ccc3c4c56)cc(-c3cc4cccc5oc6cccc3c6c45)c2)cc1]

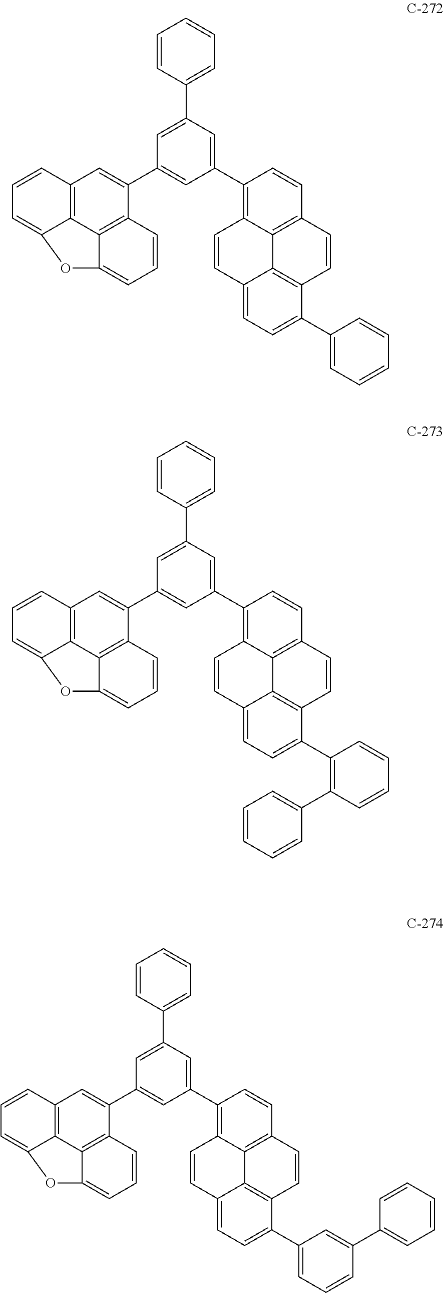 [SMILES: c1ccc(-c2cc(-c3ccc4ccc5c(-c6ccccc6)ccc6ccc3c4c65)cc(-c3cc4cccc5oc6cccc3c6c45)c2)cc1 ; c1ccc(-c2cc(-c3ccc4ccc5c(-c6ccccc6-c6ccccc6)ccc6ccc3c4c65)cc(-c3cc4cccc5oc6cccc3c6c45)c2)cc1 ; c1ccc(-c2cccc(-c3ccc4ccc5c(-c6cc(-c7ccccc7)cc(-c7cc8cccc9oc%10cccc7c%10c89)c6)ccc6ccc3c4c65)c2)cc1]

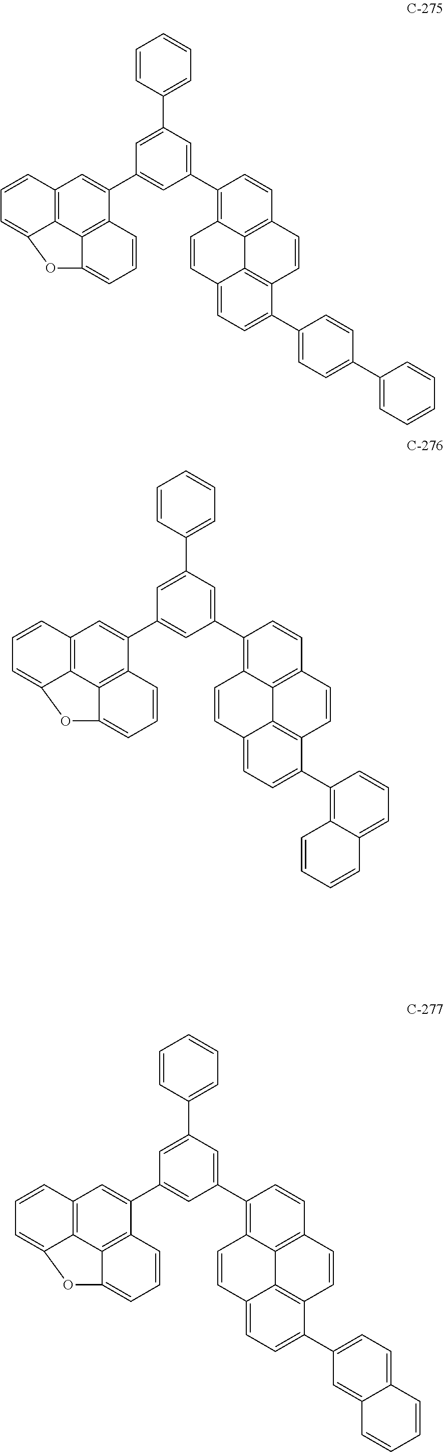 [SMILES: c1ccc(-c2ccc(-c3ccc4ccc5c(-c6cc(-c7ccccc7)cc(-c7cc8cccc9oc%10cccc7c%10c89)c6)ccc6ccc3c4c65)cc2)cc1 ; c1ccc(-c2cc(-c3ccc4ccc5c(-c6cccc7ccccc67)ccc6ccc3c4c65)cc(-c3cc4cccc5oc6cccc3c6c45)c2)cc1 ; c1ccc(-c2cc(-c3ccc4ccc5c(-c6ccc7ccccc7c6)ccc6ccc3c4c65)cc(-c3cc4cccc5oc6cccc3c6c45)c2)cc1]

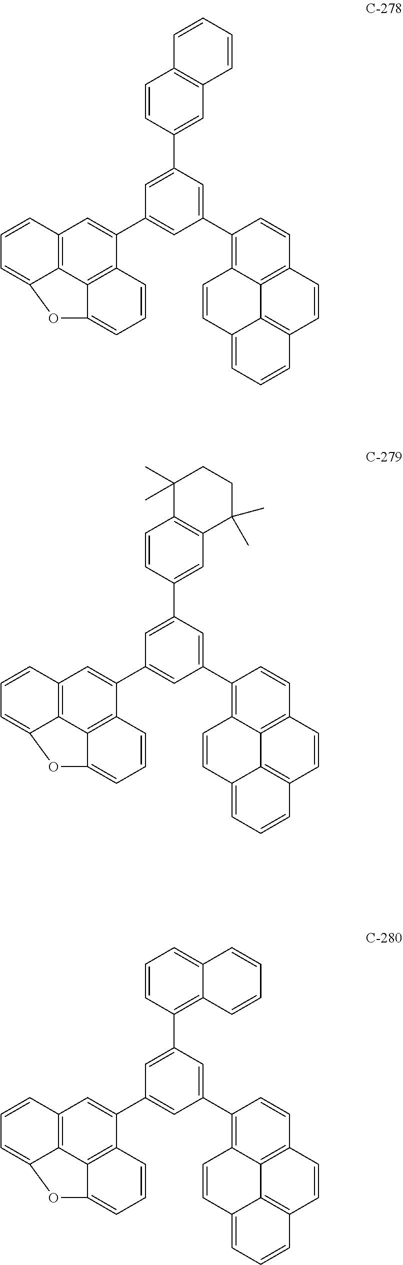 [SMILES: c1ccc2cc(-c3cc(-c4ccc5ccc6cccc7ccc4c5c67)cc(-c4cc5cccc6oc7cccc4c7c56)c3)ccc2c1 ; CC1(C)CCC(C)(C)c2cc(-c3cc(-c4ccc5ccc6cccc7ccc4c5c67)cc(-c4cc5cccc6oc7cccc4c7c56)c3)ccc21 ; c1ccc2c(-c3cc(-c4ccc5ccc6cccc7ccc4c5c67)cc(-c4cc5cccc6oc7cccc4c7c56)c3)cccc2c1]

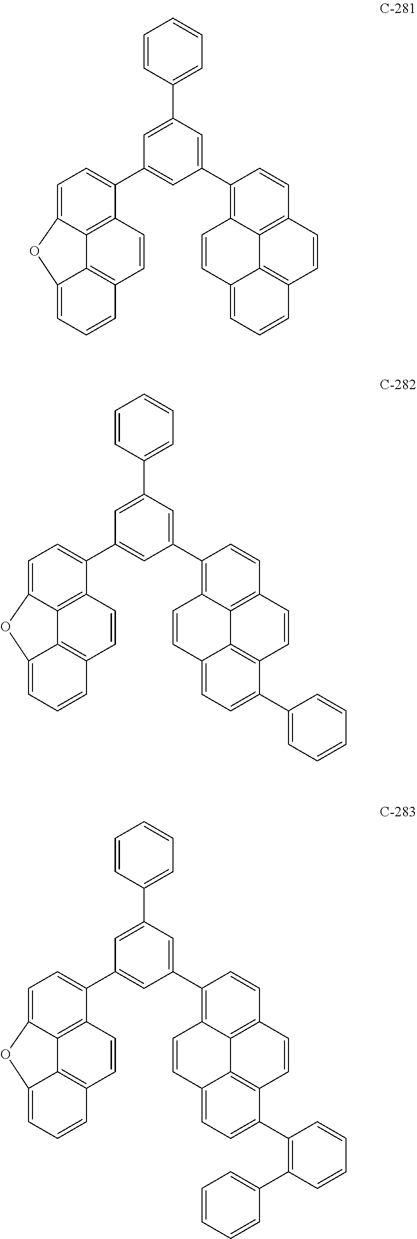 [SMILES: c1ccc(-c2cc(-c3ccc4ccc5cccc6ccc3c4c56)cc(-c3ccc4oc5cccc6ccc3c4c65)c2)cc1 ; c1ccc(-c2cc(-c3ccc4ccc5c(-c6ccccc6)ccc6ccc3c4c65)cc(-c3ccc4oc5cccc6ccc3c4c65)c2)cc1 ; c1ccc(-c2cc(-c3ccc4ccc5c(-c6ccccc6-c6ccccc6)ccc6ccc3c4c65)cc(-c3ccc4oc5cccc6ccc3c4c65)c2)cc1]

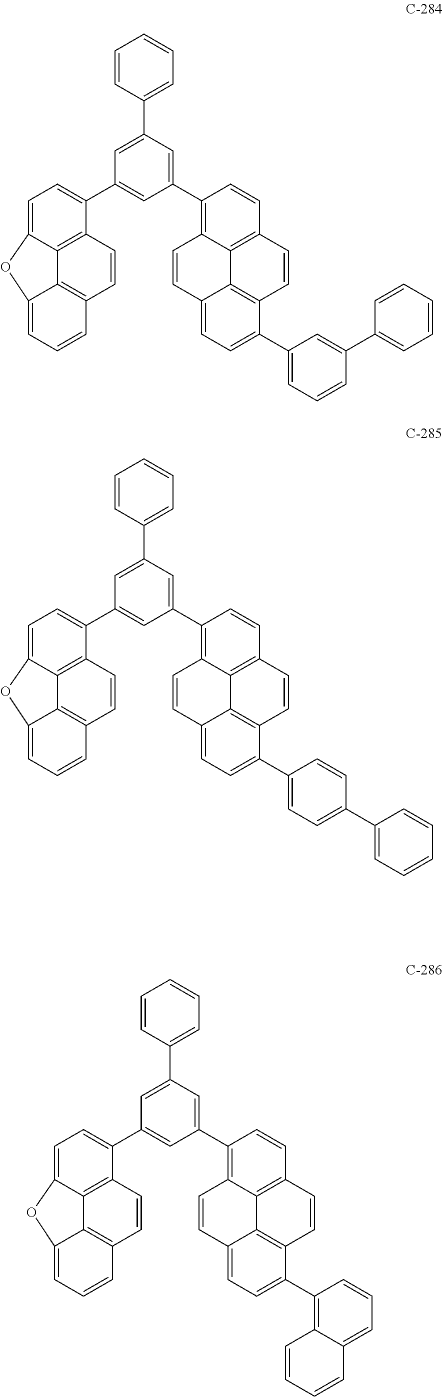 [SMILES: c1ccc(-c2cccc(-c3ccc4ccc5c(-c6cc(-c7ccccc7)cc(-c7ccc8oc9cccc%10ccc7c8c%109)c6)ccc6ccc3c4c65)c2)cc1 ; c1ccc(-c2ccc(-c3ccc4ccc5c(-c6cc(-c7ccccc7)cc(-c7ccc8oc9cccc%10ccc7c8c%109)c6)ccc6ccc3c4c65)cc2)cc1 ; c1ccc(-c2cc(-c3ccc4ccc5c(-c6cccc7ccccc67)ccc6ccc3c4c65)cc(-c3ccc4oc5cccc6ccc3c4c65)c2)cc1]

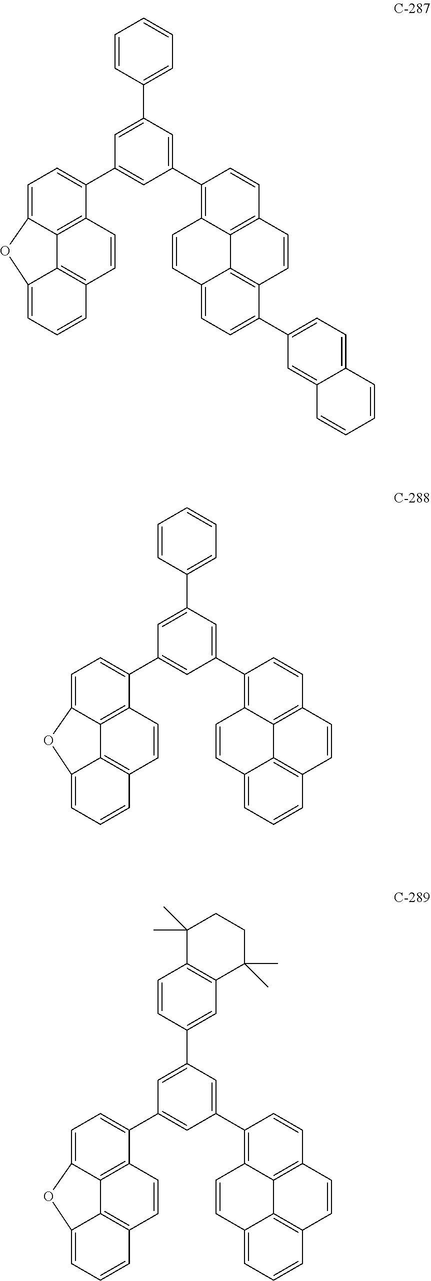 [SMILES: c1ccc(-c2cc(-c3ccc4ccc5c(-c6ccc7ccccc7c6)ccc6ccc3c4c65)cc(-c3ccc4oc5cccc6ccc3c4c65)c2)cc1 ; c1ccc(-c2cc(-c3ccc4ccc5cccc6ccc3c4c56)cc(-c3ccc4oc5cccc6ccc3c4c65)c2)cc1 ; CC1(C)CCC(C)(C)c2cc(-c3cc(-c4ccc5ccc6cccc7ccc4c5c67)cc(-c4ccc5oc6cccc7ccc4c5c76)c3)ccc21]

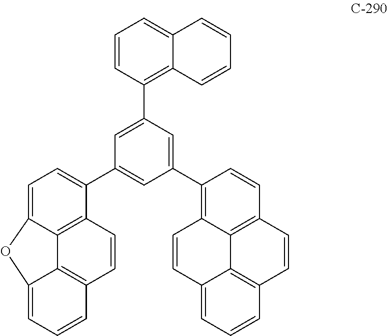 [SMILES: c1ccc2c(-c3cc(-c4ccc5ccc6cccc7ccc4c5c67)cc(-c4ccc5oc6cccc7ccc4c5c76)c3)cccc2c1]

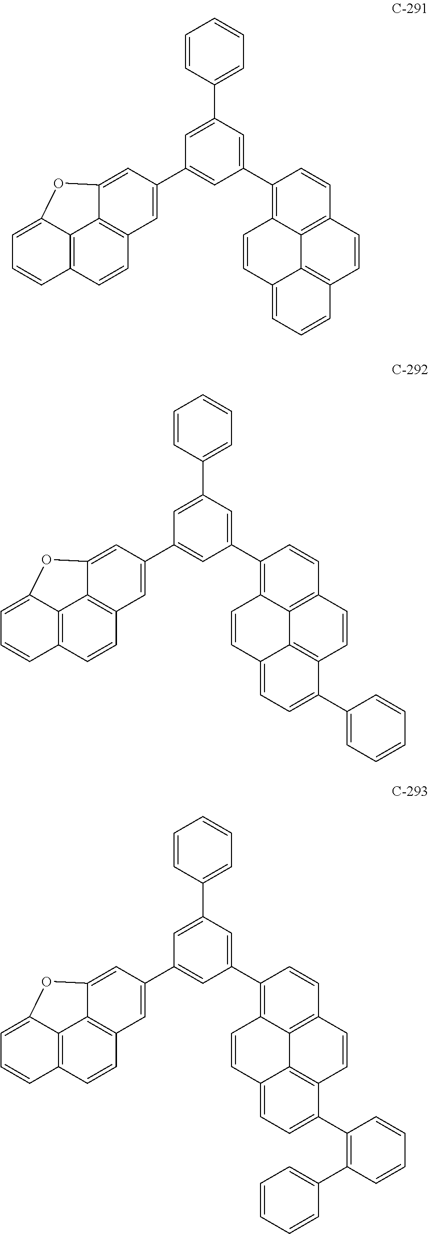 [SMILES: c1ccc(-c2cc(-c3cc4ccc5cccc6oc(c3)c4c56)cc(-c3ccc4ccc5cccc6ccc3c4c56)c2)cc1 ; c1ccc(-c2cc(-c3cc4ccc5cccc6oc(c3)c4c56)cc(-c3ccc4ccc5c(-c6ccccc6)ccc6ccc3c4c65)c2)cc1 ; c1ccc(-c2cc(-c3cc4ccc5cccc6oc(c3)c4c56)cc(-c3ccc4ccc5c(-c6ccccc6-c6ccccc6)ccc6ccc3c4c65)c2)cc1]

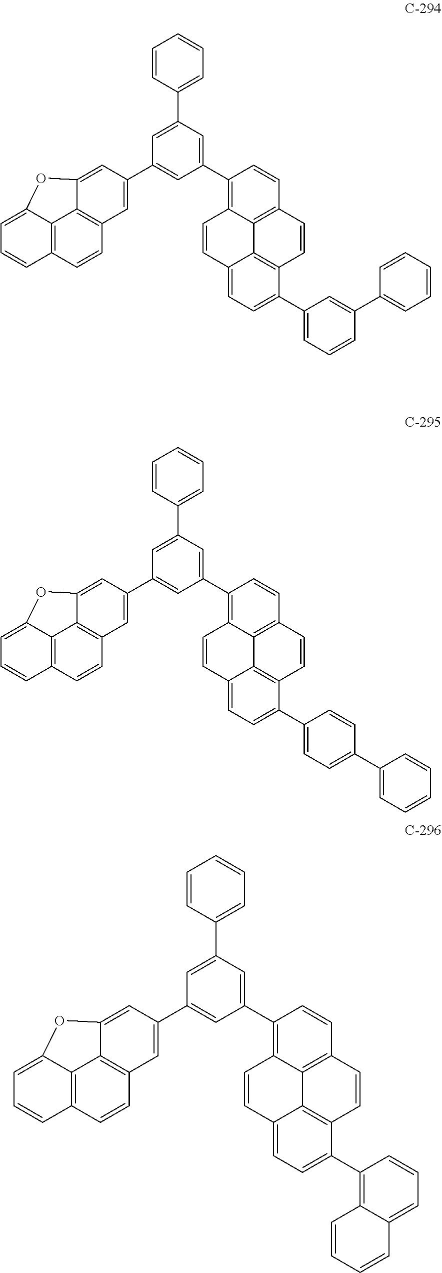 [SMILES: c1ccc(-c2cccc(-c3ccc4ccc5c(-c6cc(-c7ccccc7)cc(-c7cc8ccc9cccc%10oc(c7)c8c9%10)c6)ccc6ccc3c4c65)c2)cc1 ; c1ccc(-c2ccc(-c3ccc4ccc5c(-c6cc(-c7ccccc7)cc(-c7cc8ccc9cccc%10oc(c7)c8c9%10)c6)ccc6ccc3c4c65)cc2)cc1 ; c1ccc(-c2cc(-c3cc4ccc5cccc6oc(c3)c4c56)cc(-c3ccc4ccc5c(-c6cccc7ccccc67)ccc6ccc3c4c65)c2)cc1]

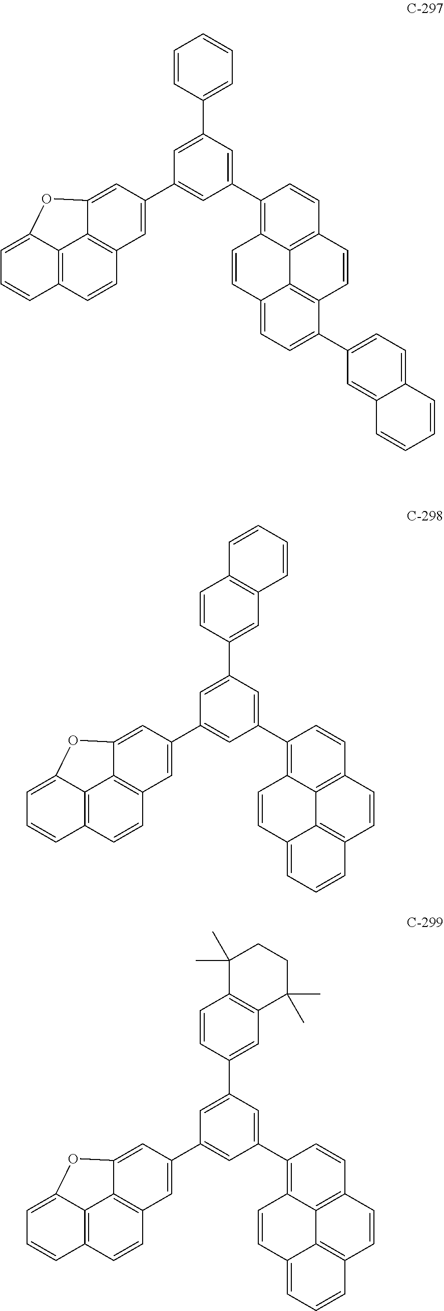 [SMILES: c1ccc(-c2cc(-c3cc4ccc5cccc6oc(c3)c4c56)cc(-c3ccc4ccc5c(-c6ccc7ccccc7c6)ccc6ccc3c4c65)c2)cc1 ; c1ccc2cc(-c3cc(-c4cc5ccc6cccc7oc(c4)c5c67)cc(-c4ccc5ccc6cccc7ccc4c5c67)c3)ccc2c1 ; CC1(C)CCC(C)(C)c2cc(-c3cc(-c4cc5ccc6cccc7oc(c4)c5c67)cc(-c4ccc5ccc6cccc7ccc4c5c67)c3)ccc21]

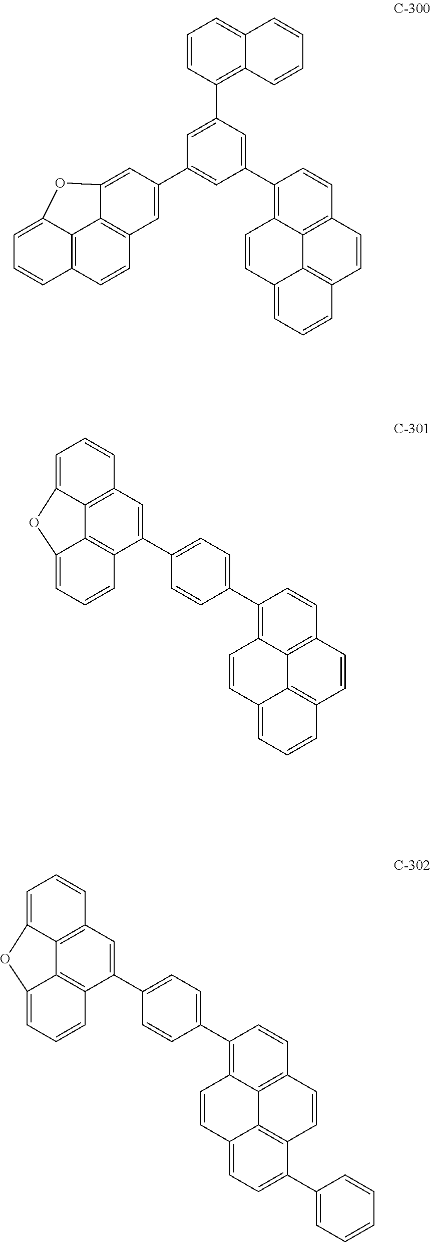 [SMILES: c1ccc2c(-c3cc(-c4cc5ccc6cccc7oc(c4)c5c67)cc(-c4ccc5ccc6cccc7ccc4c5c67)c3)cccc2c1 ; c1cc2ccc3ccc(-c4ccc(-c5cc6cccc7oc8cccc5c8c67)cc4)c4ccc(c1)c2c34 ; c1ccc(-c2ccc3ccc4c(-c5ccc(-c6cc7cccc8oc9cccc6c9c78)cc5)ccc5ccc2c3c54)cc1]

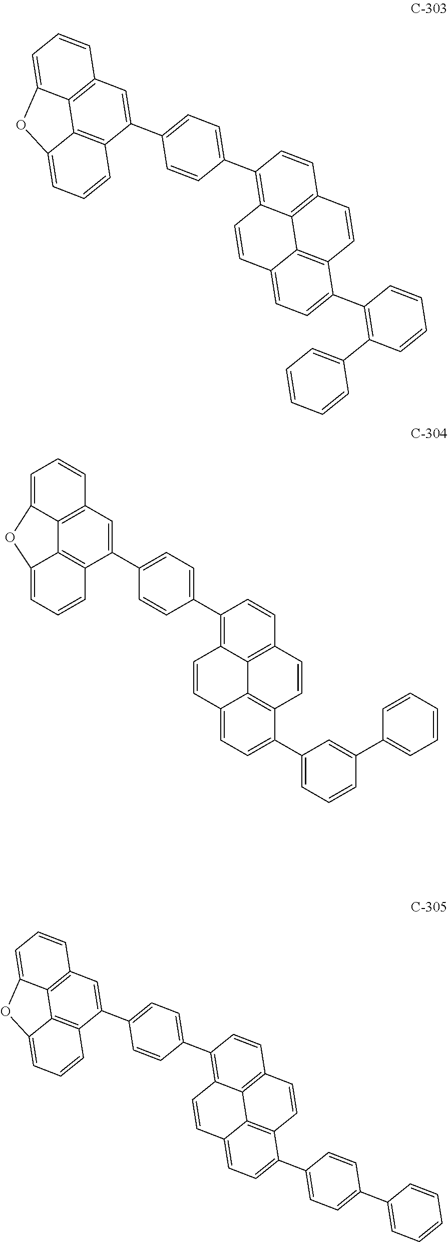 [SMILES: c1ccc(-c2ccccc2-c2ccc3ccc4c(-c5ccc(-c6cc7cccc8oc9cccc6c9c78)cc5)ccc5ccc2c3c54)cc1 ; c1ccc(-c2cccc(-c3ccc4ccc5c(-c6ccc(-c7cc8cccc9oc%10cccc7c%10c89)cc6)ccc6ccc3c4c65)c2)cc1 ; c1ccc(-c2ccc(-c3ccc4ccc5c(-c6ccc(-c7cc8cccc9oc%10cccc7c%10c89)cc6)ccc6ccc3c4c65)cc2)cc1]

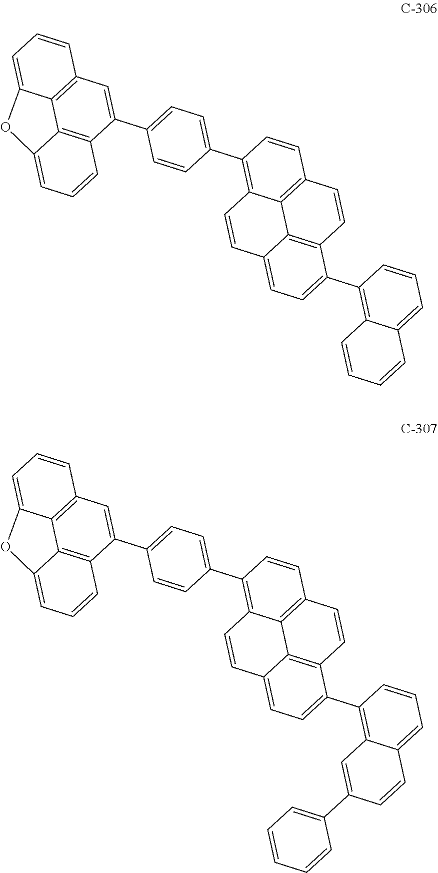 [SMILES: c1ccc2c(-c3ccc4ccc5c(-c6ccc(-c7cc8cccc9oc%10cccc7c%10c89)cc6)ccc6ccc3c4c65)cccc2c1 ; c1ccc(-c2ccc3cccc(-c4ccc5ccc6c(-c7ccc(-c8cc9cccc%10oc%11cccc8c%11c9%10)cc7)ccc7ccc4c5c76)c3c2)cc1]

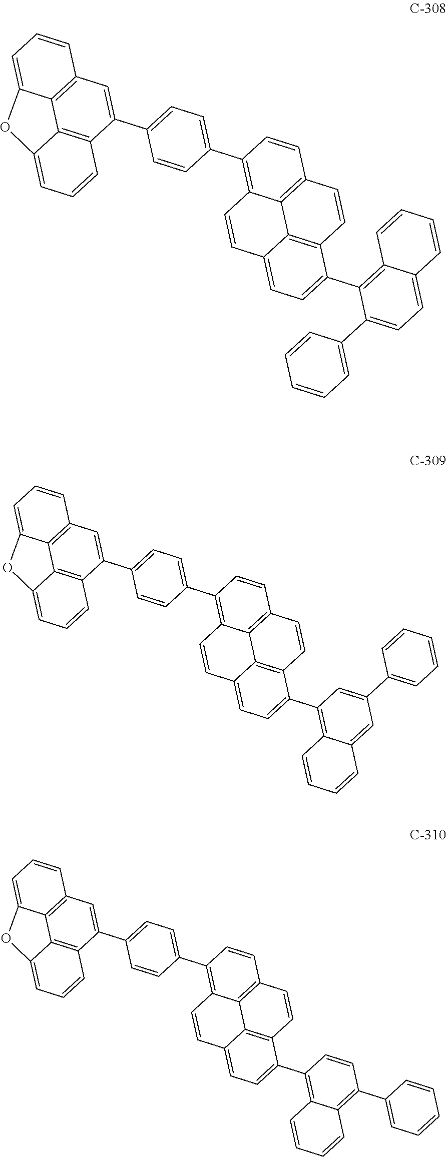 [SMILES: c1ccc(-c2ccc3ccccc3c2-c2ccc3ccc4c(-c5ccc(-c6cc7cccc8oc9cccc6c9c78)cc5)ccc5ccc2c3c54)cc1 ; c1ccc(-c2cc(-c3ccc4ccc5c(-c6ccc(-c7cc8cccc9oc%10cccc7c%10c89)cc6)ccc6ccc3c4c65)c3ccccc3c2)cc1 ; c1ccc(-c2ccc(-c3ccc4ccc5c(-c6ccc(-c7cc8cccc9oc%10cccc7c%10c89)cc6)ccc6ccc3c4c65)c3ccccc23)cc1]

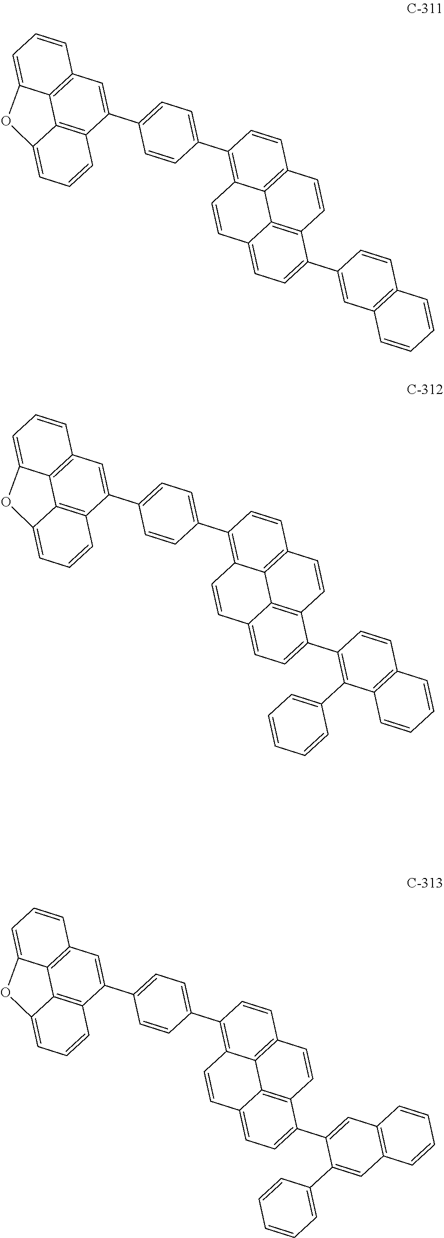 [SMILES: c1ccc2cc(-c3ccc4ccc5c(-c6ccc(-c7cc8cccc9oc%10cccc7c%10c89)cc6)ccc6ccc3c4c65)ccc2c1 ; c1ccc(-c2c(-c3ccc4ccc5c(-c6ccc(-c7cc8cccc9oc%10cccc7c%10c89)cc6)ccc6ccc3c4c65)ccc3ccccc23)cc1 ; c1ccc(-c2cc3ccccc3cc2-c2ccc3ccc4c(-c5ccc(-c6cc7cccc8oc9cccc6c9c78)cc5)ccc5ccc2c3c54)cc1]

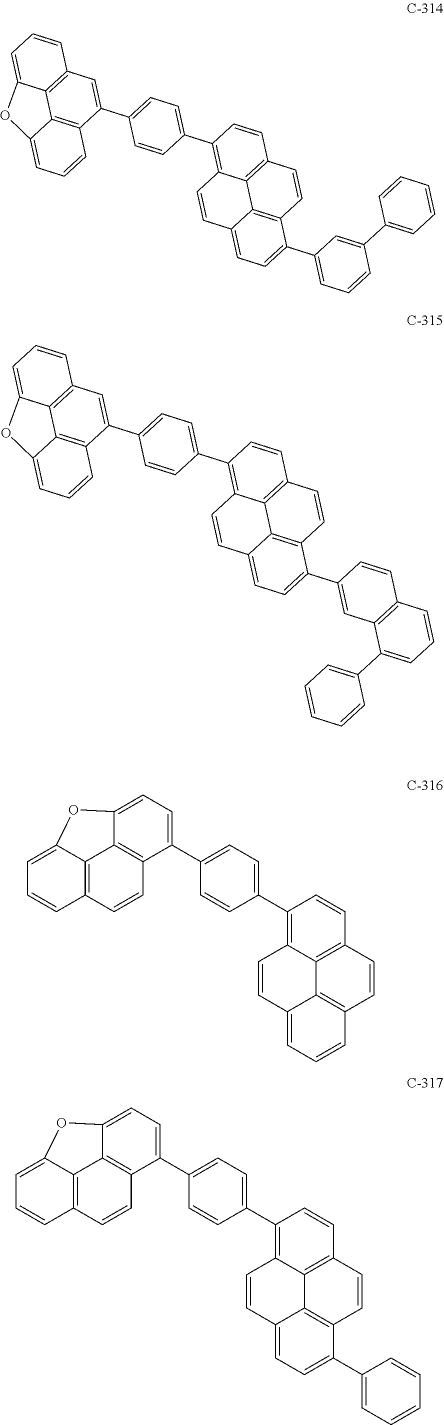 [SMILES: c1ccc(-c2cccc(-c3ccc4ccc5c(-c6ccc(-c7cc8cccc9oc%10cccc7c%10c89)cc6)ccc6ccc3c4c65)c2)cc1 ; c1ccc(-c2cccc3ccc(-c4ccc5ccc6c(-c7ccc(-c8cc9cccc%10oc%11cccc8c%11c9%10)cc7)ccc7ccc4c5c76)cc23)cc1 ; c1cc2ccc3ccc(-c4ccc(-c5ccc6oc7cccc8ccc5c6c87)cc4)c4ccc(c1)c2c34 ; c1ccc(-c2ccc3ccc4c(-c5ccc(-c6ccc7oc8cccc9ccc6c7c98)cc5)ccc5ccc2c3c54)cc1]

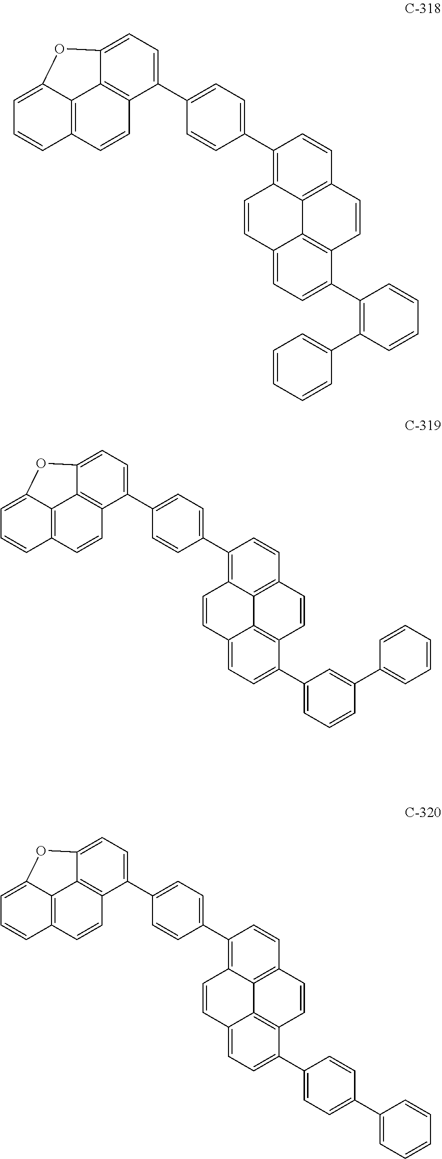 [SMILES: c1ccc(-c2ccccc2-c2ccc3ccc4c(-c5ccc(-c6ccc7oc8cccc9ccc6c7c98)cc5)ccc5ccc2c3c54)cc1 ; c1ccc(-c2cccc(-c3ccc4ccc5c(-c6ccc(-c7ccc8oc9cccc%10ccc7c8c%109)cc6)ccc6ccc3c4c65)c2)cc1 ; c1ccc(-c2ccc(-c3ccc4ccc5c(-c6ccc(-c7ccc8oc9cccc%10ccc7c8c%109)cc6)ccc6ccc3c4c65)cc2)cc1]

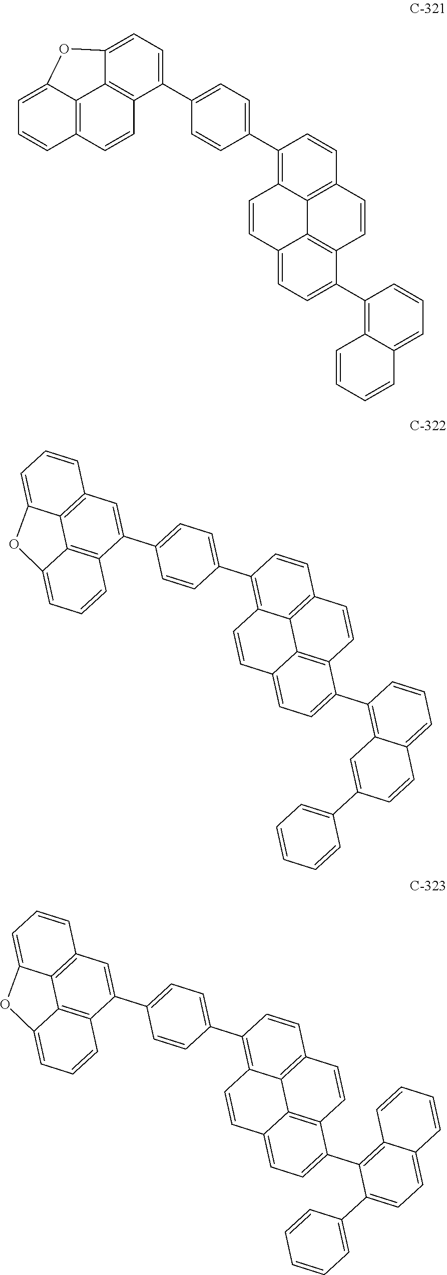 [SMILES: c1ccc2c(-c3ccc4ccc5c(-c6ccc(-c7ccc8oc9cccc%10ccc7c8c%109)cc6)ccc6ccc3c4c65)cccc2c1 ; c1ccc(-c2ccc3cccc(-c4ccc5ccc6c(-c7ccc(-c8cc9cccc%10oc%11cccc8c%11c9%10)cc7)ccc7ccc4c5c76)c3c2)cc1 ; c1ccc(-c2ccc3ccccc3c2-c2ccc3ccc4c(-c5ccc(-c6cc7cccc8oc9cccc6c9c78)cc5)ccc5ccc2c3c54)cc1]

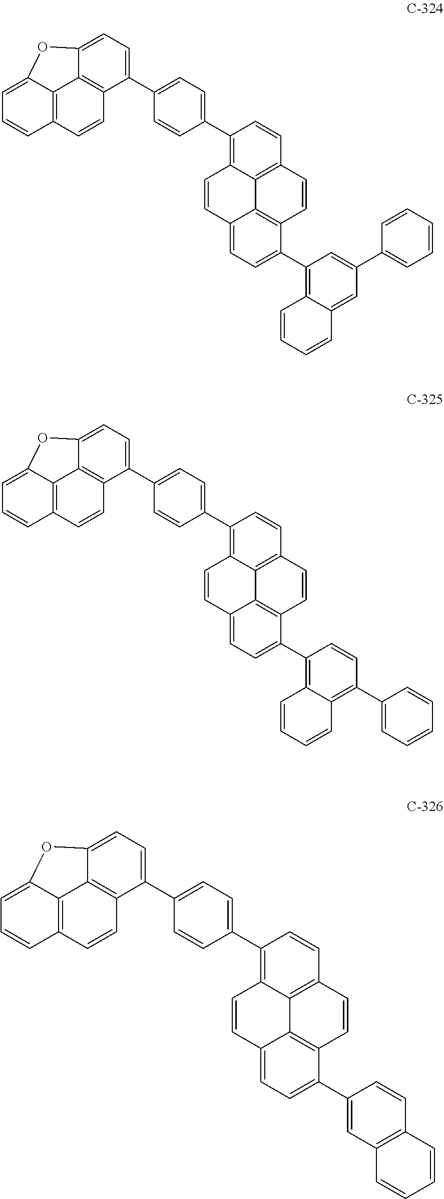 [SMILES: c1ccc(-c2cc(-c3ccc4ccc5c(-c6ccc(-c7ccc8oc9cccc%10ccc7c8c%109)cc6)ccc6ccc3c4c65)c3ccccc3c2)cc1 ; c1ccc(-c2ccc(-c3ccc4ccc5c(-c6ccc(-c7ccc8oc9cccc%10ccc7c8c%109)cc6)ccc6ccc3c4c65)c3ccccc23)cc1 ; c1ccc2cc(-c3ccc4ccc5c(-c6ccc(-c7ccc8oc9cccc%10ccc7c8c%109)cc6)ccc6ccc3c4c65)ccc2c1]

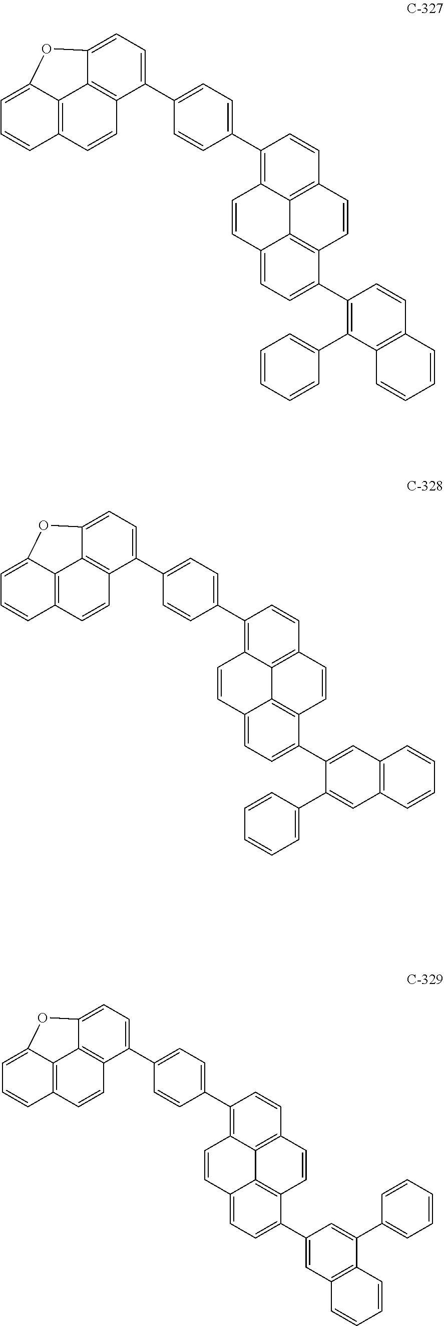 [SMILES: c1ccc(-c2c(-c3ccc4ccc5c(-c6ccc(-c7ccc8oc9cccc%10ccc7c8c%109)cc6)ccc6ccc3c4c65)ccc3ccccc23)cc1 ; c1ccc(-c2cc3ccccc3cc2-c2ccc3ccc4c(-c5ccc(-c6ccc7oc8cccc9ccc6c7c98)cc5)ccc5ccc2c3c54)cc1 ; c1ccc(-c2cc(-c3ccc4ccc5c(-c6ccc(-c7ccc8oc9cccc%10ccc7c8c%109)cc6)ccc6ccc3c4c65)cc3ccccc23)cc1]

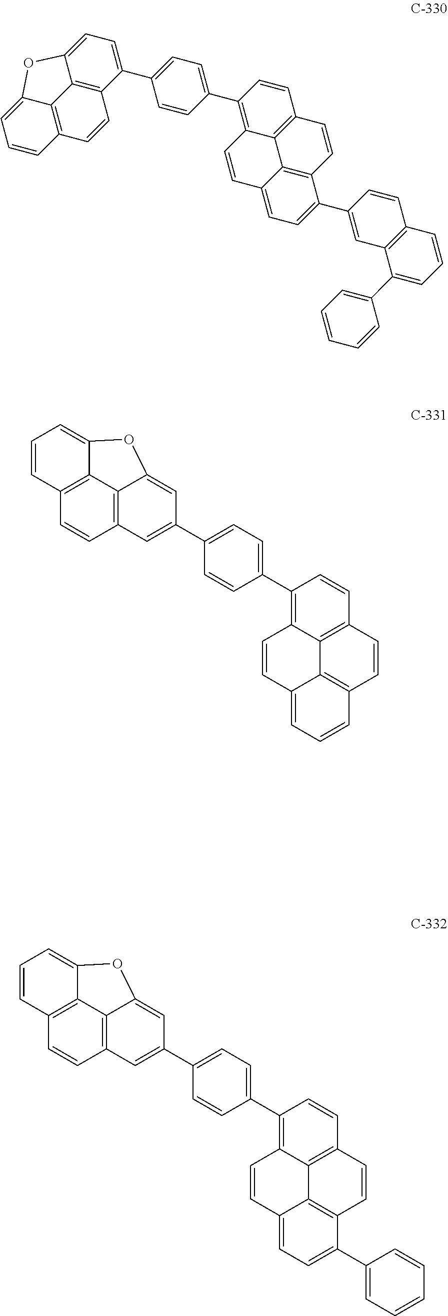 [SMILES: c1ccc(-c2cccc3ccc(-c4ccc5ccc6c(-c7ccc(-c8ccc9oc%10cccc%11ccc8c9c%11%10)cc7)ccc7ccc4c5c76)cc23)cc1 ; c1cc2ccc3ccc(-c4ccc(-c5cc6ccc7cccc8oc(c5)c6c78)cc4)c4ccc(c1)c2c34 ; c1ccc(-c2ccc3ccc4c(-c5ccc(-c6cc7ccc8cccc9oc(c6)c7c89)cc5)ccc5ccc2c3c54)cc1]

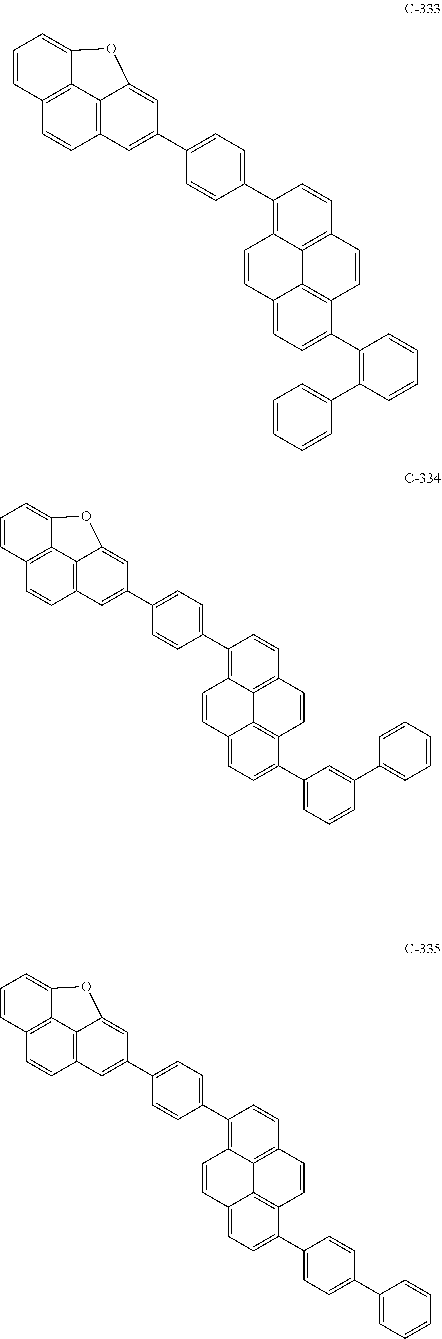 [SMILES: c1ccc(-c2ccccc2-c2ccc3ccc4c(-c5ccc(-c6cc7ccc8cccc9oc(c6)c7c89)cc5)ccc5ccc2c3c54)cc1 ; c1ccc(-c2cccc(-c3ccc4ccc5c(-c6ccc(-c7cc8ccc9cccc%10oc(c7)c8c9%10)cc6)ccc6ccc3c4c65)c2)cc1 ; c1ccc(-c2ccc(-c3ccc4ccc5c(-c6ccc(-c7cc8ccc9cccc%10oc(c7)c8c9%10)cc6)ccc6ccc3c4c65)cc2)cc1]

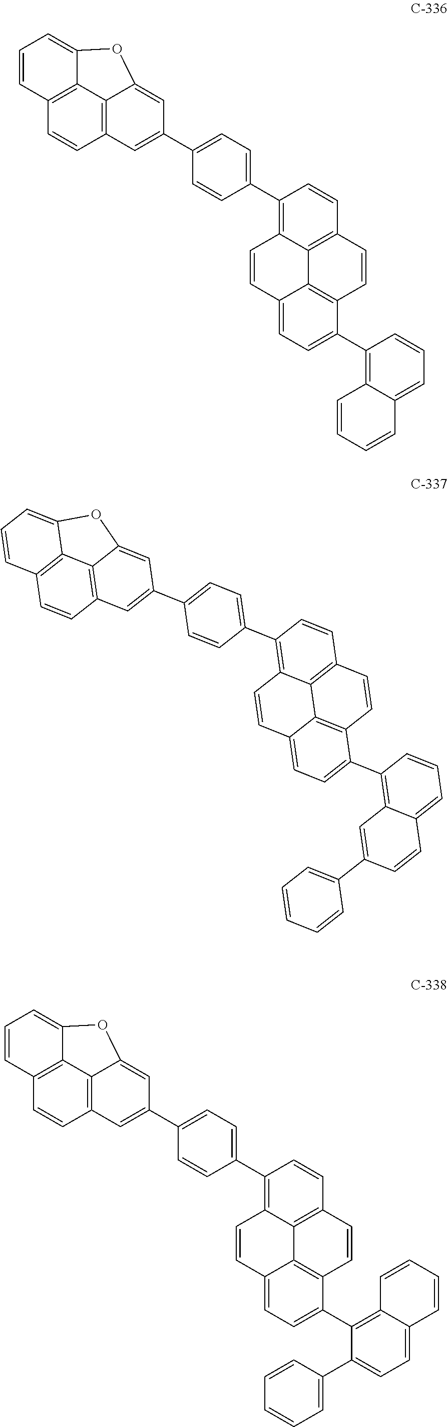 [SMILES: c1ccc2c(-c3ccc4ccc5c(-c6ccc(-c7cc8ccc9cccc%10oc(c7)c8c9%10)cc6)ccc6ccc3c4c65)cccc2c1 ; c1ccc(-c2ccc3cccc(-c4ccc5ccc6c(-c7ccc(-c8cc9ccc%10cccc%11oc(c8)c9c%10%11)cc7)ccc7ccc4c5c76)c3c2)cc1 ; c1ccc(-c2ccc3ccccc3c2-c2ccc3ccc4c(-c5ccc(-c6cc7ccc8cccc9oc(c6)c7c89)cc5)ccc5ccc2c3c54)cc1]

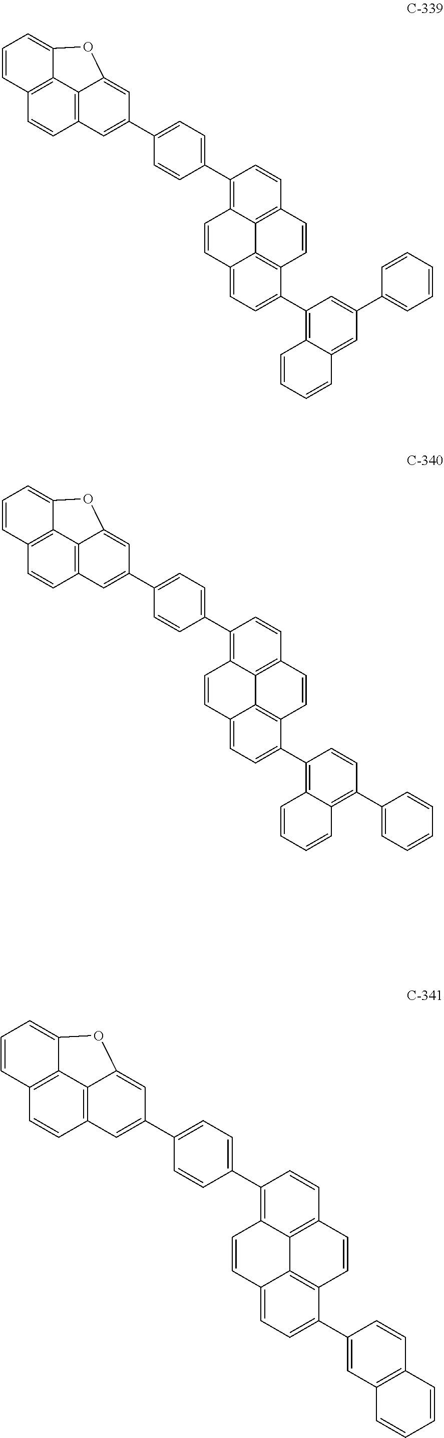 [SMILES: c1ccc(-c2cc(-c3ccc4ccc5c(-c6ccc(-c7cc8ccc9cccc%10oc(c7)c8c9%10)cc6)ccc6ccc3c4c65)c3ccccc3c2)cc1 ; c1ccc(-c2ccc(-c3ccc4ccc5c(-c6ccc(-c7cc8ccc9cccc%10oc(c7)c8c9%10)cc6)ccc6ccc3c4c65)c3ccccc23)cc1 ; c1ccc2cc(-c3ccc4ccc5c(-c6ccc(-c7cc8ccc9cccc%10oc(c7)c8c9%10)cc6)ccc6ccc3c4c65)ccc2c1]

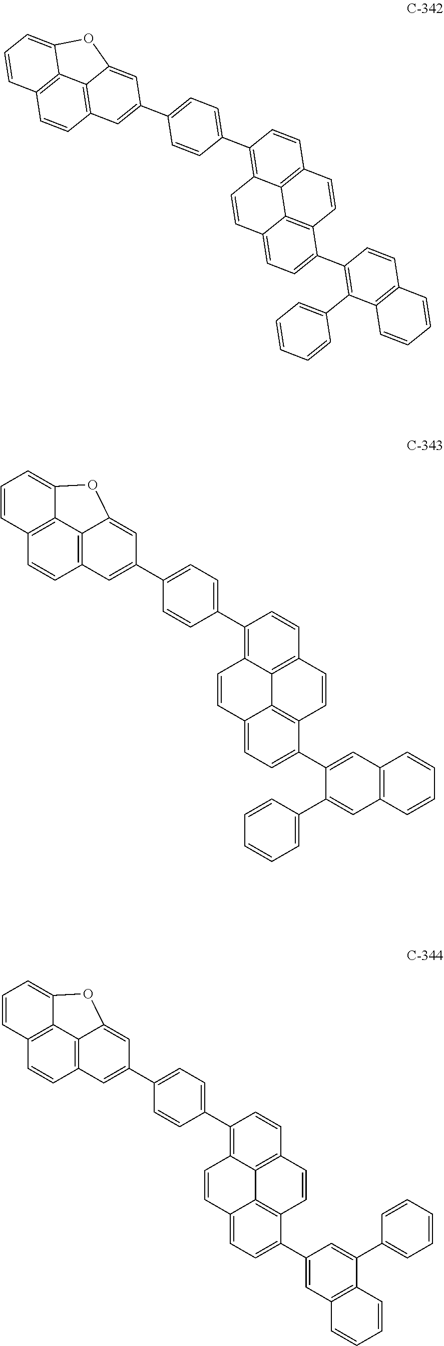 [SMILES: c1ccc(-c2c(-c3ccc4ccc5c(-c6ccc(-c7cc8ccc9cccc%10oc(c7)c8c9%10)cc6)ccc6ccc3c4c65)ccc3ccccc23)cc1 ; c1ccc(-c2cc3ccccc3cc2-c2ccc3ccc4c(-c5ccc(-c6cc7ccc8cccc9oc(c6)c7c89)cc5)ccc5ccc2c3c54)cc1 ; c1ccc(-c2cc(-c3ccc4ccc5c(-c6ccc(-c7cc8ccc9cccc%10oc(c7)c8c9%10)cc6)ccc6ccc3c4c65)cc3ccccc23)cc1]

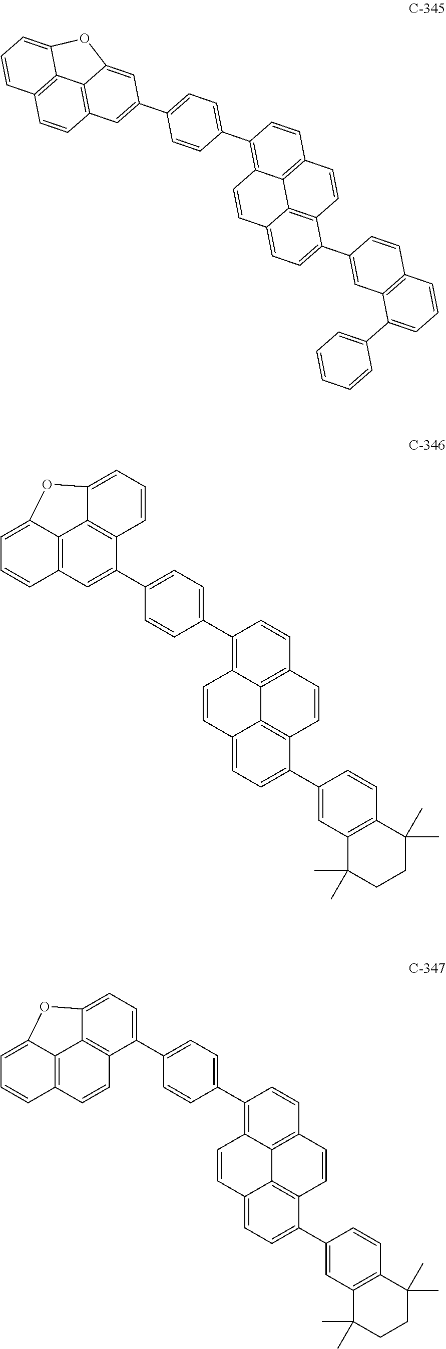 [SMILES: c1ccc(-c2cccc3ccc(-c4ccc5ccc6c(-c7ccc(-c8cc9ccc%10cccc%11oc(c8)c9c%10%11)cc7)ccc7ccc4c5c76)cc23)cc1 ; CC1(C)CCC(C)(C)c2cc(-c3ccc4ccc5c(-c6ccc(-c7cc8cccc9oc%10cccc7c%10c89)cc6)ccc6ccc3c4c65)ccc21 ; CC1(C)CCC(C)(C)c2cc(-c3ccc4ccc5c(-c6ccc(-c7ccc8oc9cccc%10ccc7c8c%109)cc6)ccc6ccc3c4c65)ccc21]

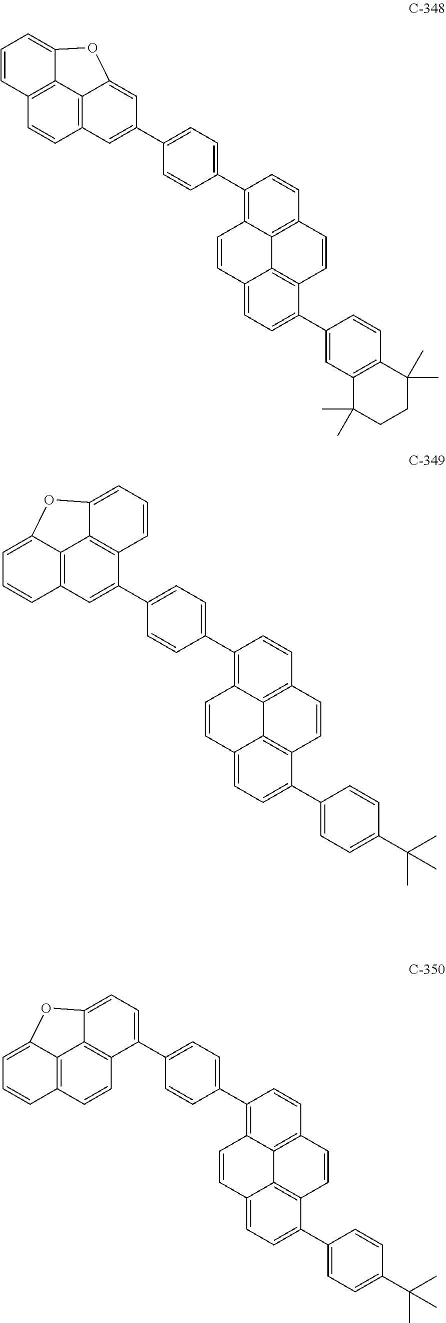 [SMILES: CC1(C)CCC(C)(C)c2cc(-c3ccc4ccc5c(-c6ccc(-c7cc8ccc9cccc%10oc(c7)c8c9%10)cc6)ccc6ccc3c4c65)ccc21 ; CC(C)(C)c1ccc(-c2ccc3ccc4c(-c5ccc(-c6cc7cccc8oc9cccc6c9c78)cc5)ccc5ccc2c3c54)cc1 ; CC(C)(C)c1ccc(-c2ccc3ccc4c(-c5ccc(-c6ccc7oc8cccc9ccc6c7c98)cc5)ccc5ccc2c3c54)cc1]

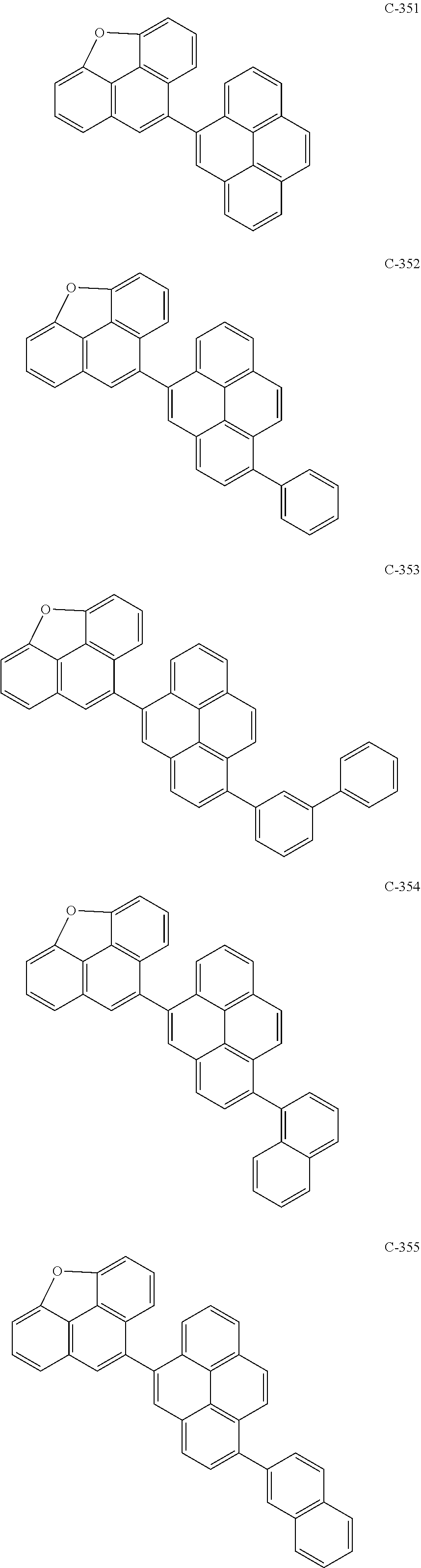 [SMILES: c1cc2ccc3cccc4c(-c5cc6cccc7oc8cccc5c8c67)cc(c1)c2c34 ; c1ccc(-c2ccc3cc(-c4cc5cccc6oc7cccc4c7c56)c4cccc5ccc2c3c54)cc1 ; c1ccc(-c2cccc(-c3ccc4cc(-c5cc6cccc7oc8cccc5c8c67)c5cccc6ccc3c4c65)c2)cc1 ; c1ccc2c(-c3ccc4cc(-c5cc6cccc7oc8cccc5c8c67)c5cccc6ccc3c4c65)cccc2c1 ; c1ccc2cc(-c3ccc4cc(-c5cc6cccc7oc8cccc5c8c67)c5cccc6ccc3c4c65)ccc2c1]

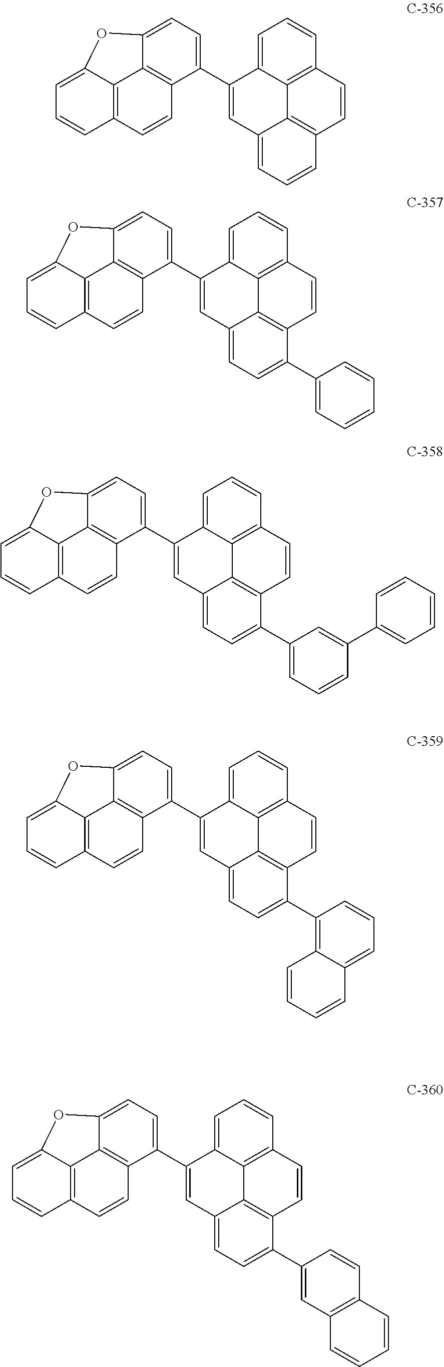

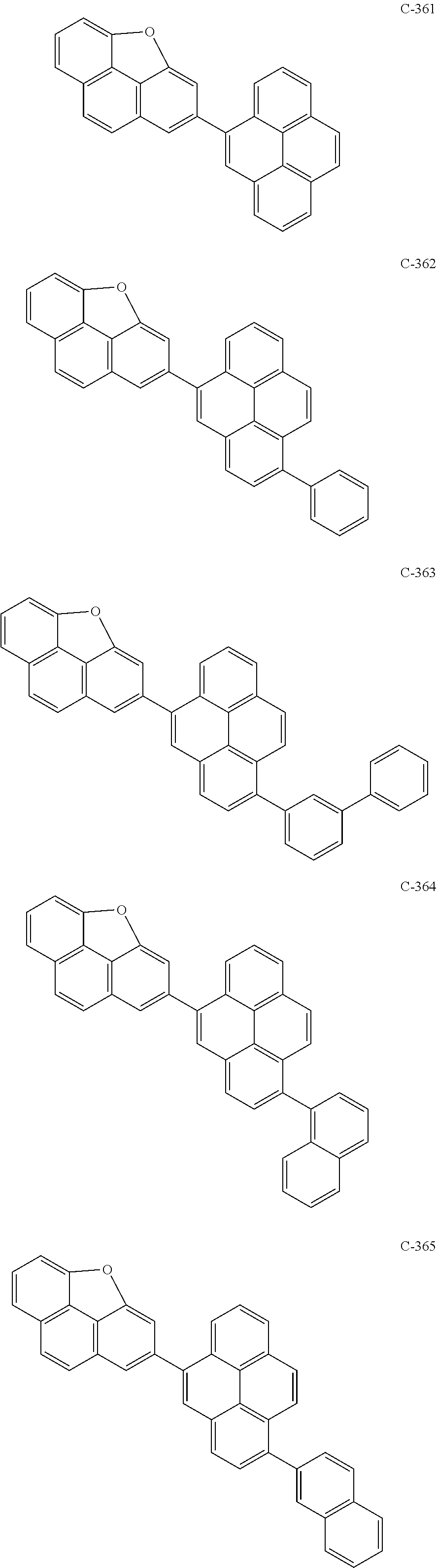 [SMILES: c1cc2ccc3cccc4c(-c5cc6ccc7cccc8oc(c5)c6c78)cc(c1)c2c34 ; c1ccc(-c2ccc3cc(-c4cc5ccc6cccc7oc(c4)c5c67)c4cccc5ccc2c3c54)cc1 ; c1ccc(-c2cccc(-c3ccc4cc(-c5cc6ccc7cccc8oc(c5)c6c78)c5cccc6ccc3c4c65)c2)cc1 ; c1ccc2c(-c3ccc4cc(-c5cc6ccc7cccc8oc(c5)c6c78)c5cccc6ccc3c4c65)cccc2c1 ; c1ccc2cc(-c3ccc4cc(-c5cc6ccc7cccc8oc(c5)c6c78)c5cccc6ccc3c4c65)ccc2c1]

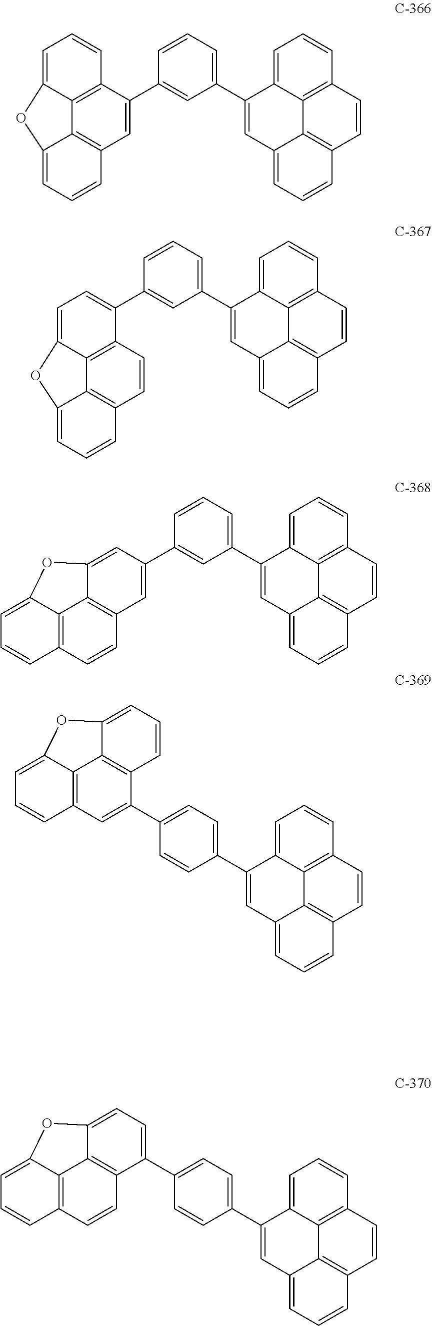

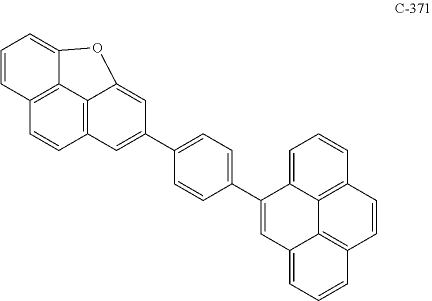 [SMILES: c1cc2ccc3cccc4c(-c5ccc(-c6cc7ccc8cccc9oc(c6)c7c89)cc5)cc(c1)c2c34]

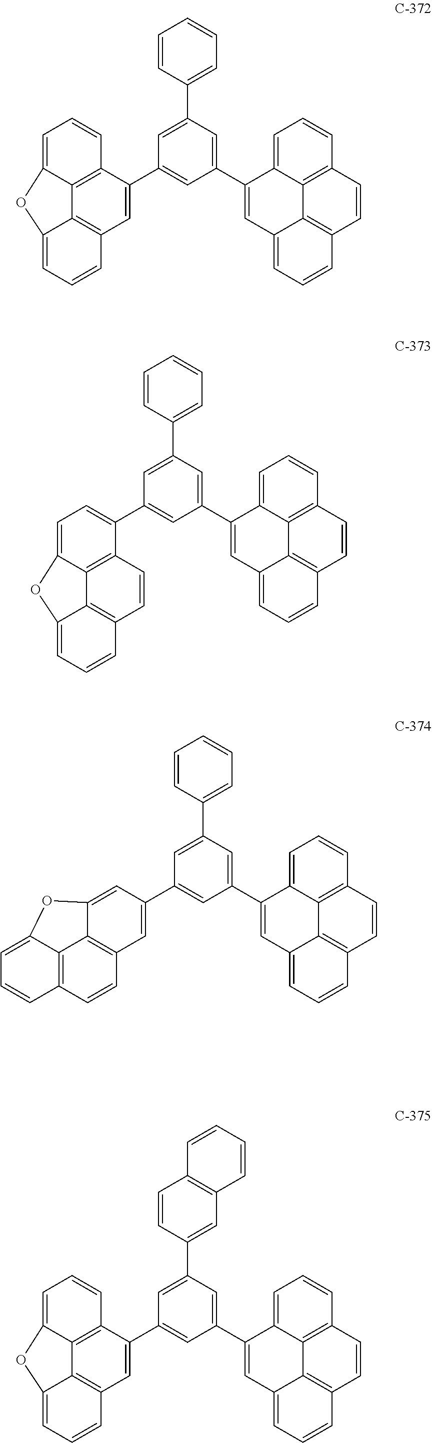 [SMILES: c1ccc(-c2cc(-c3cc4cccc5ccc6cccc3c6c54)cc(-c3cc4cccc5oc6cccc3c6c45)c2)cc1 ; c1ccc(-c2cc(-c3cc4cccc5ccc6cccc3c6c54)cc(-c3ccc4oc5cccc6ccc3c4c65)c2)cc1 ; c1ccc(-c2cc(-c3cc4ccc5cccc6oc(c3)c4c56)cc(-c3cc4cccc5ccc6cccc3c6c54)c2)cc1 ; c1ccc2cc(-c3cc(-c4cc5cccc6ccc7cccc4c7c65)cc(-c4cc5cccc6oc7cccc4c7c56)c3)ccc2c1]

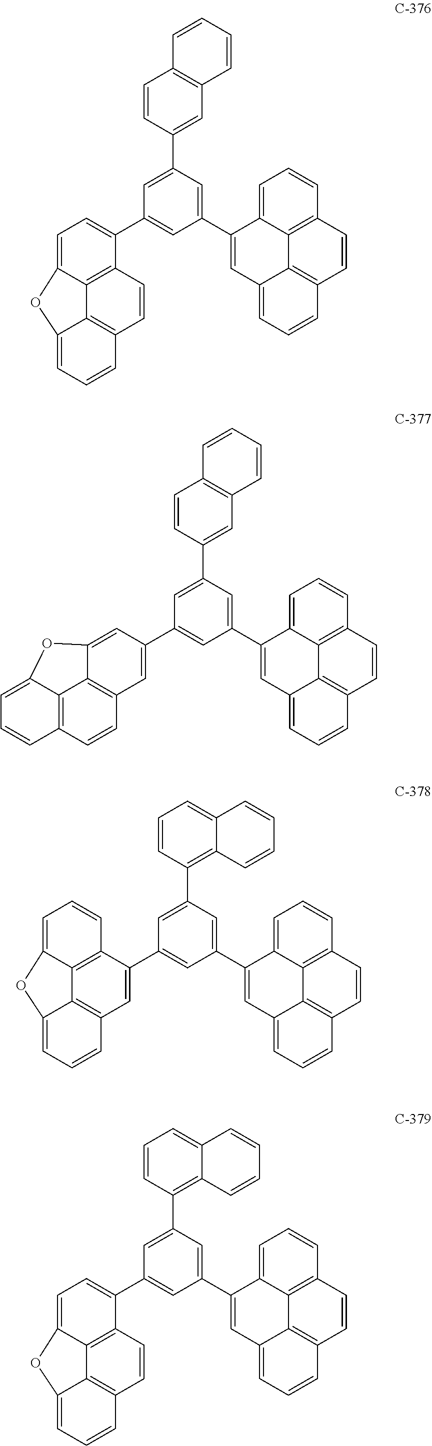 [SMILES: c1ccc2cc(-c3cc(-c4cc5cccc6ccc7cccc4c7c65)cc(-c4ccc5oc6cccc7ccc4c5c76)c3)ccc2c1 ; c1ccc2cc(-c3cc(-c4cc5ccc6cccc7oc(c4)c5c67)cc(-c4cc5cccc6ccc7cccc4c7c65)c3)ccc2c1 ; c1ccc2c(-c3cc(-c4cc5cccc6ccc7cccc4c7c65)cc(-c4cc5cccc6oc7cccc4c7c56)c3)cccc2c1 ; c1ccc2c(-c3cc(-c4cc5cccc6ccc7cccc4c7c65)cc(-c4ccc5oc6cccc7ccc4c5c76)c3)cccc2c1]

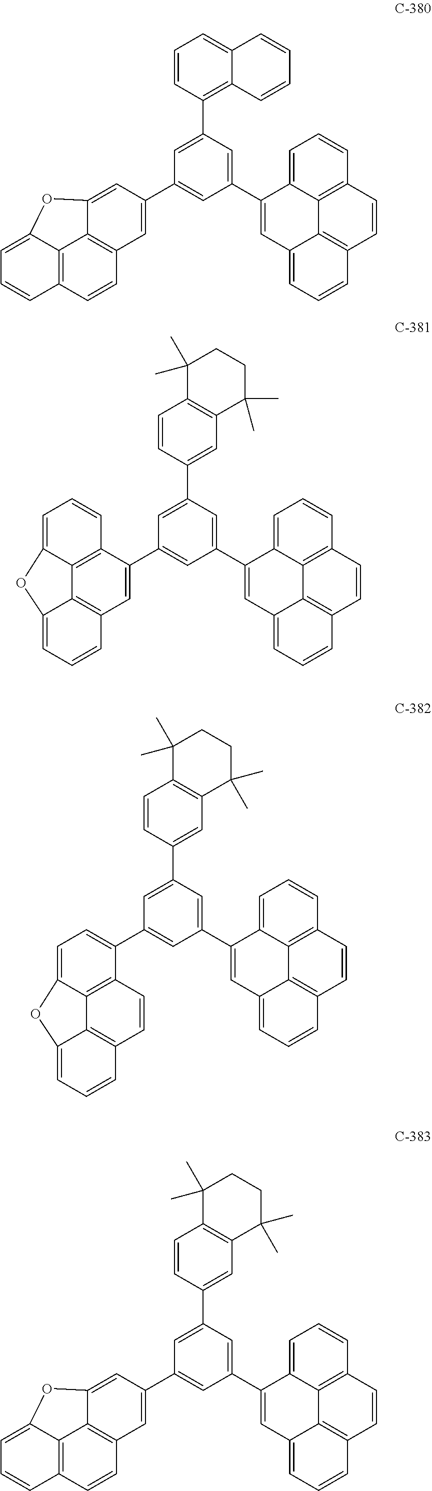 [SMILES: c1ccc2c(-c3cc(-c4cc5ccc6cccc7oc(c4)c5c67)cc(-c4cc5cccc6ccc7cccc4c7c65)c3)cccc2c1 ; CC1(C)CCC(C)(C)c2cc(-c3cc(-c4cc5cccc6ccc7cccc4c7c65)cc(-c4cc5cccc6oc7cccc4c7c56)c3)ccc21 ; CC1(C)CCC(C)(C)c2cc(-c3cc(-c4cc5cccc6ccc7cccc4c7c65)cc(-c4ccc5oc6cccc7ccc4c5c76)c3)ccc21 ; CC1(C)CCC(C)(C)c2cc(-c3cc(-c4cc5ccc6cccc7oc(c4)c5c67)cc(-c4cc5cccc6ccc7cccc4c7c65)c3)ccc21]

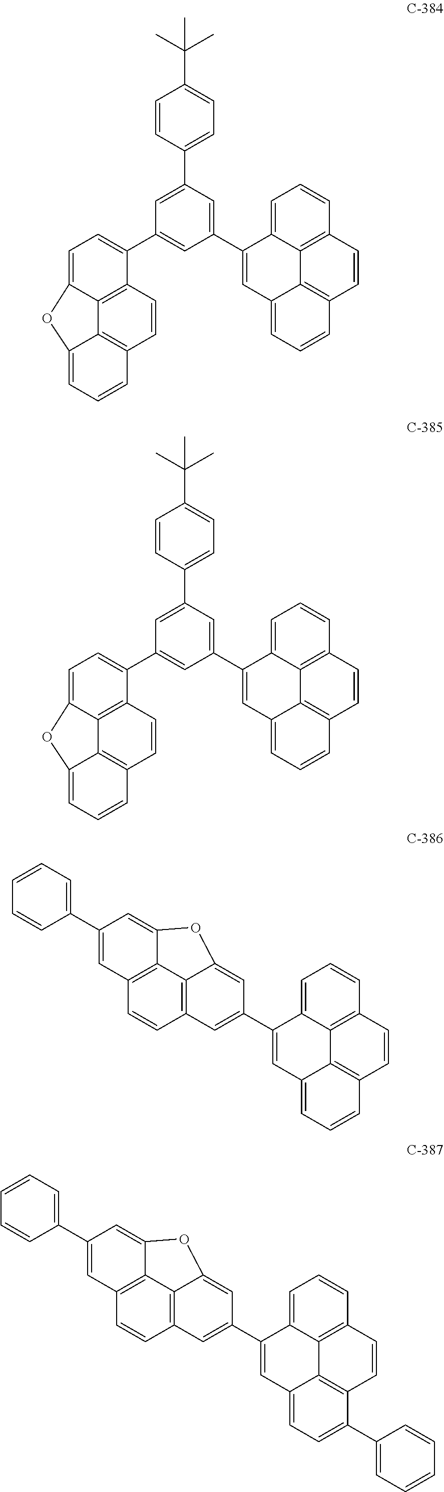 [SMILES: CC(C)(C)c1ccc(-c2cc(-c3cc4cccc5ccc6cccc3c6c54)cc(-c3ccc4oc5cccc6ccc3c4c65)c2)cc1 ; CC(C)(C)c1ccc(-c2cc(-c3cc4cccc5ccc6cccc3c6c54)cc(-c3ccc4oc5cccc6ccc3c4c65)c2)cc1 ; c1ccc(-c2cc3ccc4cc(-c5cc6cccc7ccc8cccc5c8c76)cc5oc(c2)c3c45)cc1 ; c1ccc(-c2cc3ccc4cc(-c5cc6ccc(-c7ccccc7)c7ccc8cccc5c8c67)cc5oc(c2)c3c45)cc1]

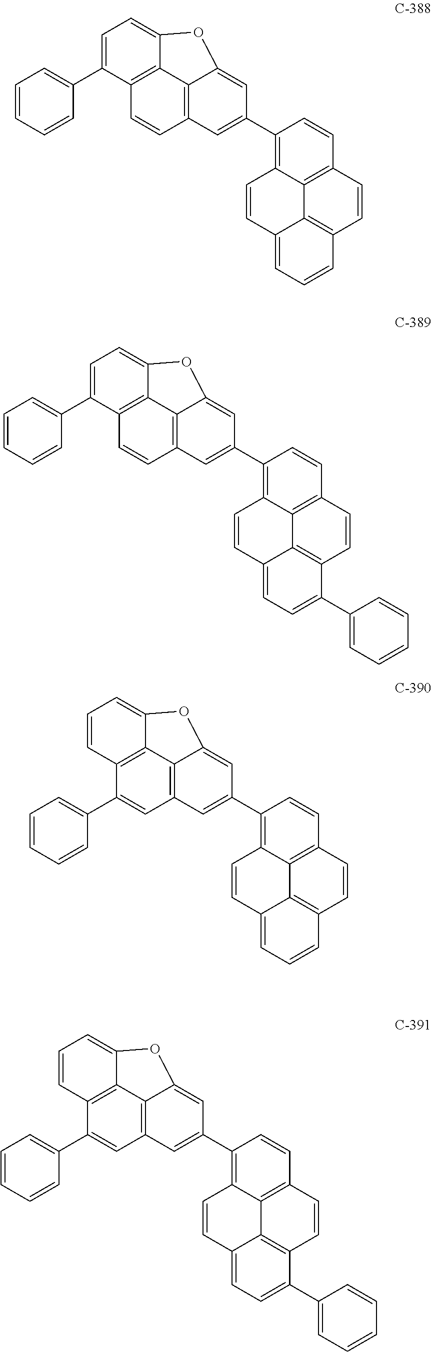 [SMILES: c1ccc(-c2ccc3oc4cc(-c5ccc6ccc7cccc8ccc5c6c78)cc5ccc2c3c54)cc1 ; c1ccc(-c2ccc3ccc4c(-c5cc6ccc7c(-c8ccccc8)ccc8oc(c5)c6c87)ccc5ccc2c3c54)cc1 ; c1ccc(-c2cc3cc(-c4ccc5ccc6cccc7ccc4c5c67)cc4oc5cccc2c5c34)cc1 ; c1ccc(-c2ccc3ccc4c(-c5cc6cc(-c7ccccc7)c7cccc8oc(c5)c6c87)ccc5ccc2c3c54)cc1]

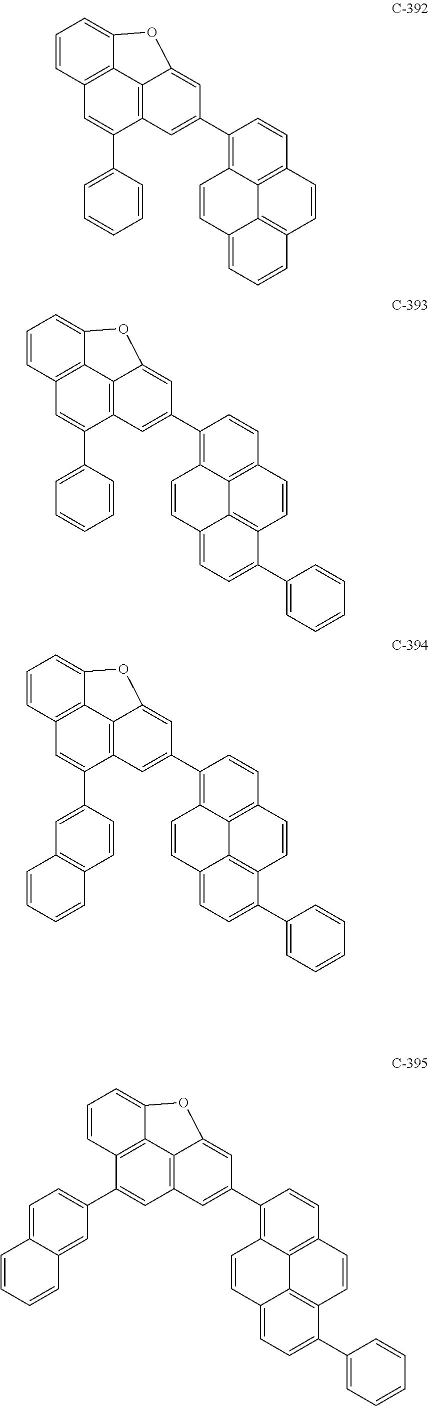 [SMILES: c1ccc(-c2cc3cccc4oc5cc(-c6ccc7ccc8cccc9ccc6c7c89)cc2c5c34)cc1 ; c1ccc(-c2ccc3ccc4c(-c5cc6oc7cccc8cc(-c9ccccc9)c(c5)c6c87)ccc5ccc2c3c54)cc1 ; c1ccc(-c2ccc3ccc4c(-c5cc6oc7cccc8cc(-c9ccc%10ccccc%10c9)c(c5)c6c87)ccc5ccc2c3c54)cc1 ; c1ccc(-c2ccc3ccc4c(-c5cc6cc(-c7ccc8ccccc8c7)c7cccc8oc(c5)c6c87)ccc5ccc2c3c54)cc1]

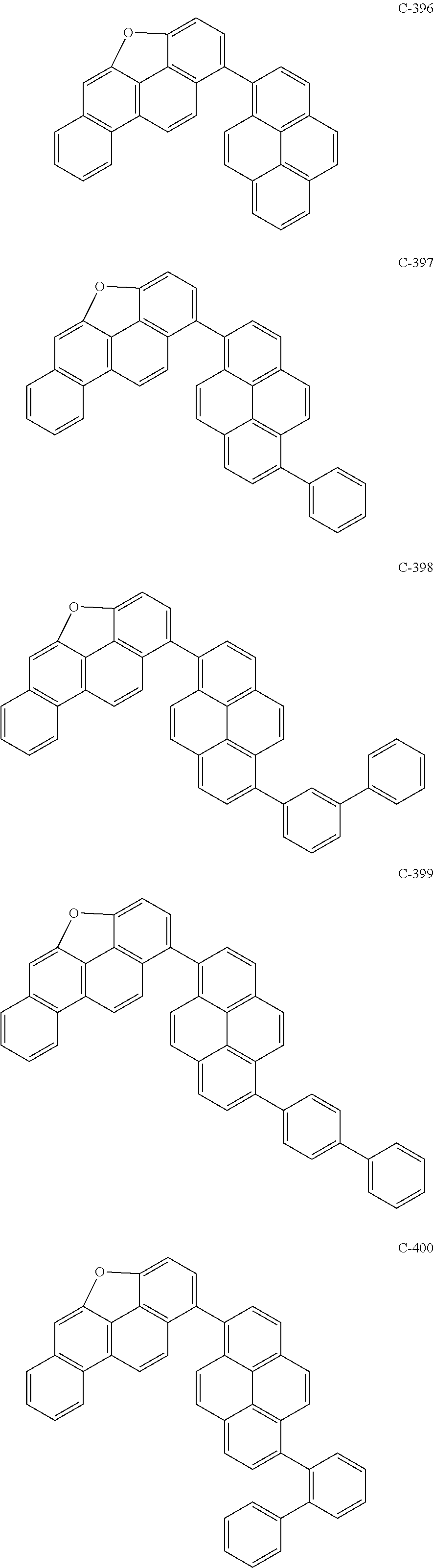 [SMILES: c1ccc2c(c1)cc1oc3ccc(-c4ccc5ccc6cccc7ccc4c5c67)c4ccc2c1c34 ; c1ccc(-c2ccc3ccc4c(-c5ccc6oc7cc8ccccc8c8ccc5c6c78)ccc5ccc2c3c54)cc1 ; c1ccc(-c2cccc(-c3ccc4ccc5c(-c6ccc7oc8cc9ccccc9c9ccc6c7c89)ccc6ccc3c4c65)c2)cc1 ; c1ccc(-c2ccc(-c3ccc4ccc5c(-c6ccc7oc8cc9ccccc9c9ccc6c7c89)ccc6ccc3c4c65)cc2)cc1 ; c1ccc(-c2ccccc2-c2ccc3ccc4c(-c5ccc6oc7cc8ccccc8c8ccc5c6c78)ccc5ccc2c3c54)cc1]

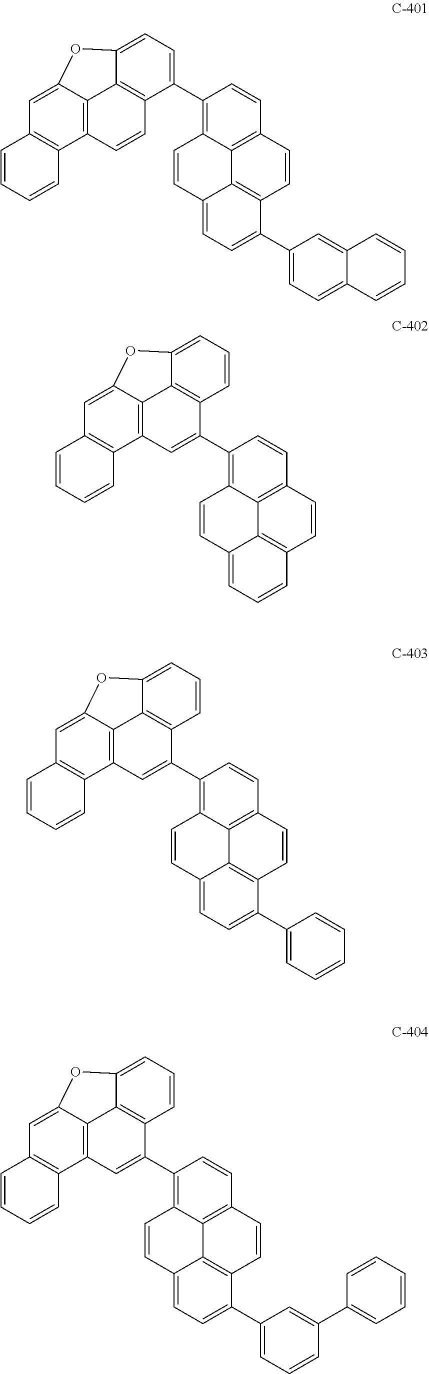 [SMILES: c1ccc2cc(-c3ccc4ccc5c(-c6ccc7oc8cc9ccccc9c9ccc6c7c89)ccc6ccc3c4c65)ccc2c1 ; c1ccc2c(c1)cc1oc3cccc4c(-c5ccc6ccc7cccc8ccc5c6c78)cc2c1c34 ; c1ccc(-c2ccc3ccc4c(-c5cc6c7ccccc7cc7oc8cccc5c8c76)ccc5ccc2c3c54)cc1 ; c1ccc(-c2cccc(-c3ccc4ccc5c(-c6cc7c8ccccc8cc8oc9cccc6c9c87)ccc6ccc3c4c65)c2)cc1]

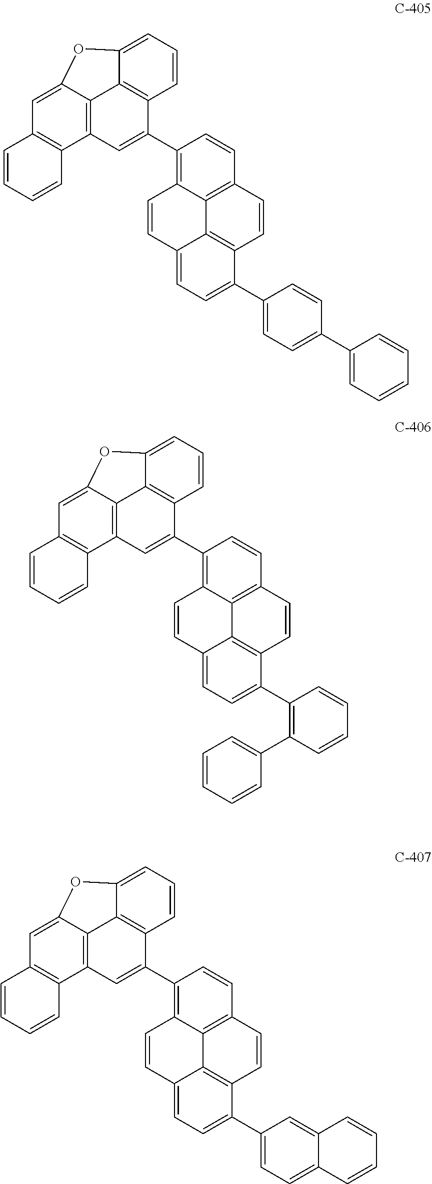 [SMILES: c1ccc(-c2ccc(-c3ccc4ccc5c(-c6cc7c8ccccc8cc8oc9cccc6c9c87)ccc6ccc3c4c65)cc2)cc1 ; c1ccc(-c2ccccc2-c2ccc3ccc4c(-c5cc6c7ccccc7cc7oc8cccc5c8c76)ccc5ccc2c3c54)cc1 ; c1ccc2cc(-c3ccc4ccc5c(-c6cc7c8ccccc8cc8oc9cccc6c9c87)ccc6ccc3c4c65)ccc2c1]

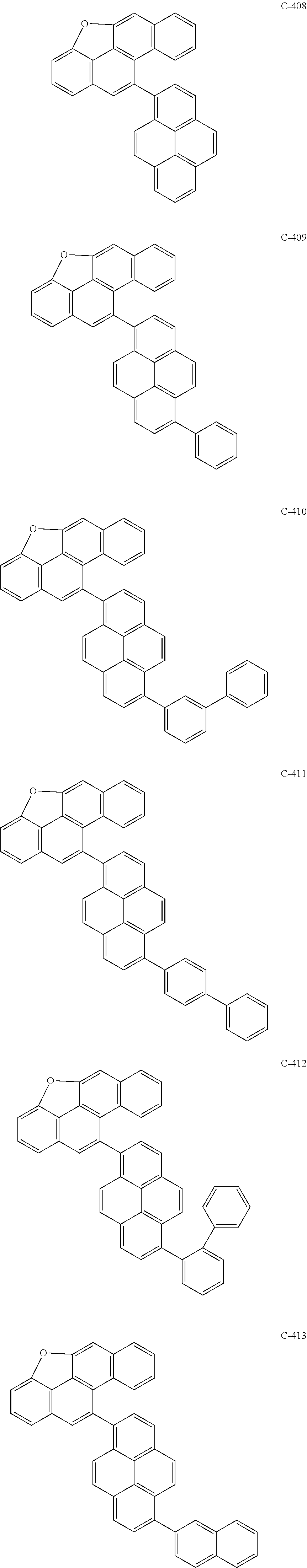 [SMILES: c1ccc2c(c1)cc1oc3cccc4cc(-c5ccc6ccc7cccc8ccc5c6c78)c2c1c43 ; c1ccc(-c2ccc3ccc4c(-c5cc6cccc7oc8cc9ccccc9c5c8c67)ccc5ccc2c3c54)cc1 ; c1ccc(-c2cccc(-c3ccc4ccc5c(-c6cc7cccc8oc9cc%10ccccc%10c6c9c78)ccc6ccc3c4c65)c2)cc1 ; c1ccc(-c2ccc(-c3ccc4ccc5c(-c6cc7cccc8oc9cc%10ccccc%10c6c9c78)ccc6ccc3c4c65)cc2)cc1 ; c1ccc(-c2ccccc2-c2ccc3ccc4c(-c5cc6cccc7oc8cc9ccccc9c5c8c67)ccc5ccc2c3c54)cc1 ; c1ccc2cc(-c3ccc4ccc5c(-c6cc7cccc8oc9cc%10ccccc%10c6c9c78)ccc6ccc3c4c65)ccc2c1]

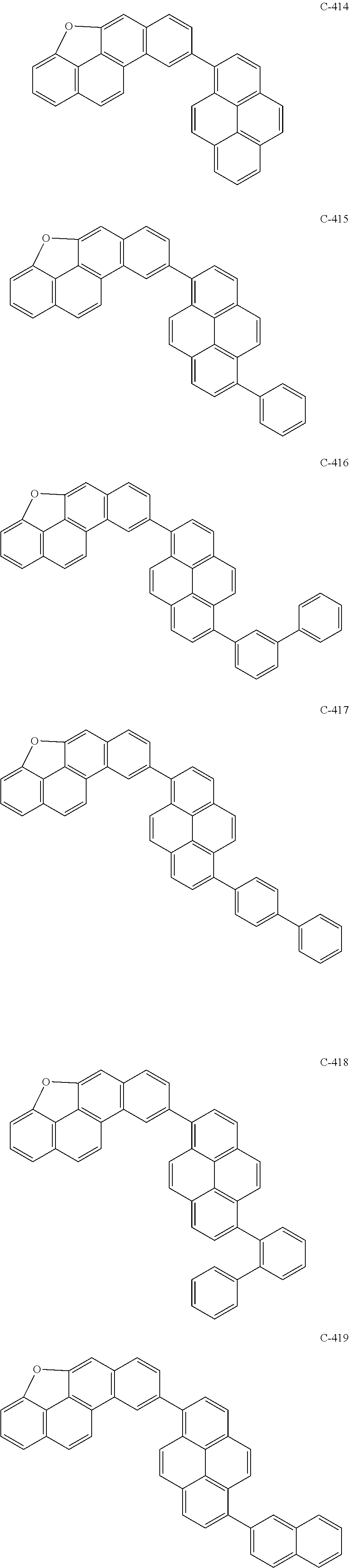 [SMILES: c1cc2ccc3ccc(-c4ccc5cc6oc7cccc8ccc(c5c4)c6c87)c4ccc(c1)c2c34 ; c1ccc(-c2ccc3ccc4c(-c5ccc6cc7oc8cccc9ccc(c6c5)c7c98)ccc5ccc2c3c54)cc1 ; c1ccc(-c2cccc(-c3ccc4ccc5c(-c6ccc7cc8oc9cccc%10ccc(c7c6)c8c%109)ccc6ccc3c4c65)c2)cc1 ; c1ccc(-c2ccc(-c3ccc4ccc5c(-c6ccc7cc8oc9cccc%10ccc(c7c6)c8c%109)ccc6ccc3c4c65)cc2)cc1 ; c1ccc(-c2ccccc2-c2ccc3ccc4c(-c5ccc6cc7oc8cccc9ccc(c6c5)c7c98)ccc5ccc2c3c54)cc1 ; c1ccc2cc(-c3ccc4ccc5c(-c6ccc7cc8oc9cccc%10ccc(c7c6)c8c%109)ccc6ccc3c4c65)ccc2c1]

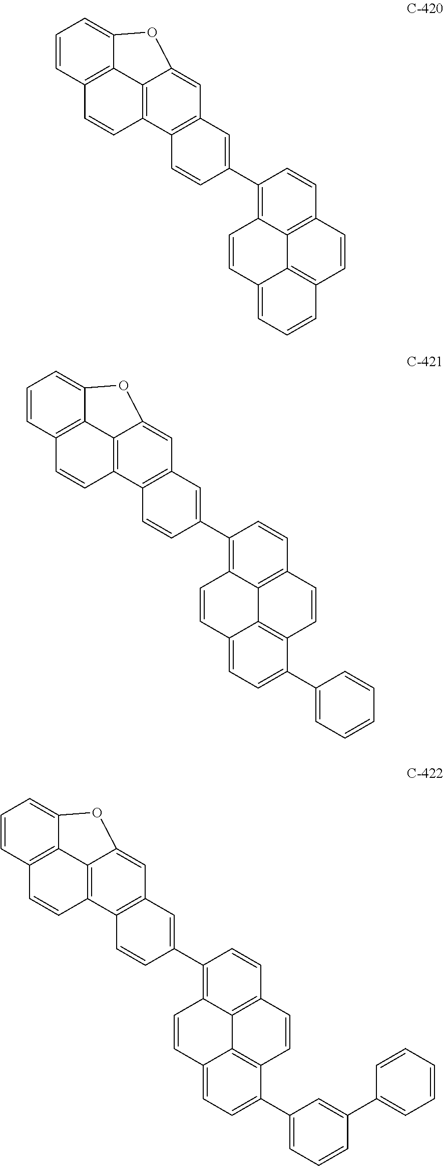 [SMILES: c1cc2ccc3ccc(-c4ccc5c(c4)cc4oc6cccc7ccc5c4c76)c4ccc(c1)c2c34 ; c1ccc(-c2ccc3ccc4c(-c5ccc6c(c5)cc5oc7cccc8ccc6c5c87)ccc5ccc2c3c54)cc1 ; c1ccc(-c2cccc(-c3ccc4ccc5c(-c6ccc7c(c6)cc6oc8cccc9ccc7c6c98)ccc6ccc3c4c65)c2)cc1]

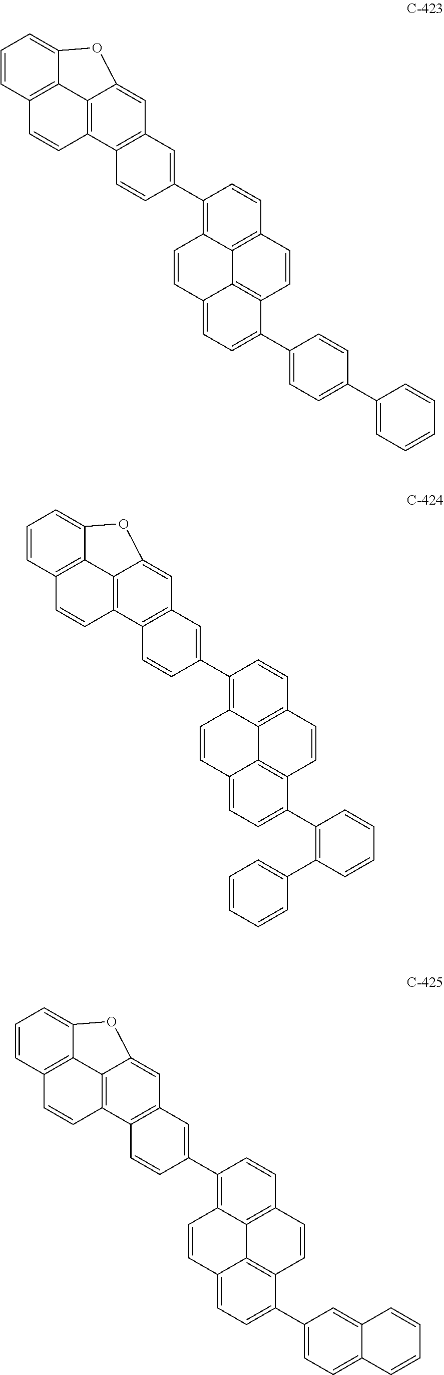 [SMILES: c1ccc(-c2ccc(-c3ccc4ccc5c(-c6ccc7c(c6)cc6oc8cccc9ccc7c6c98)ccc6ccc3c4c65)cc2)cc1 ; c1ccc(-c2ccccc2-c2ccc3ccc4c(-c5ccc6c(c5)cc5oc7cccc8ccc6c5c87)ccc5ccc2c3c54)cc1 ; c1ccc2cc(-c3ccc4ccc5c(-c6ccc7c(c6)cc6oc8cccc9ccc7c6c98)ccc6ccc3c4c65)ccc2c1]

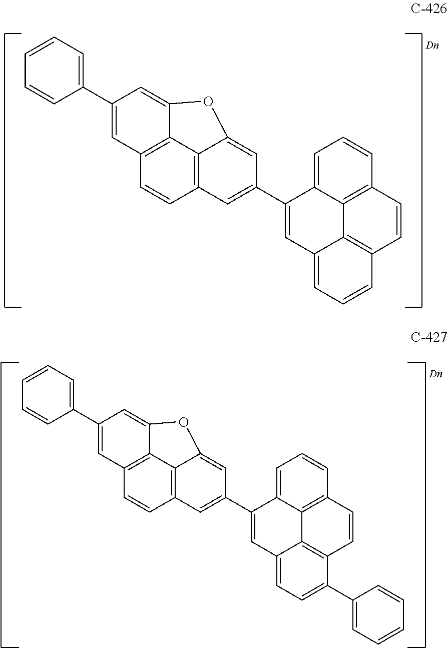

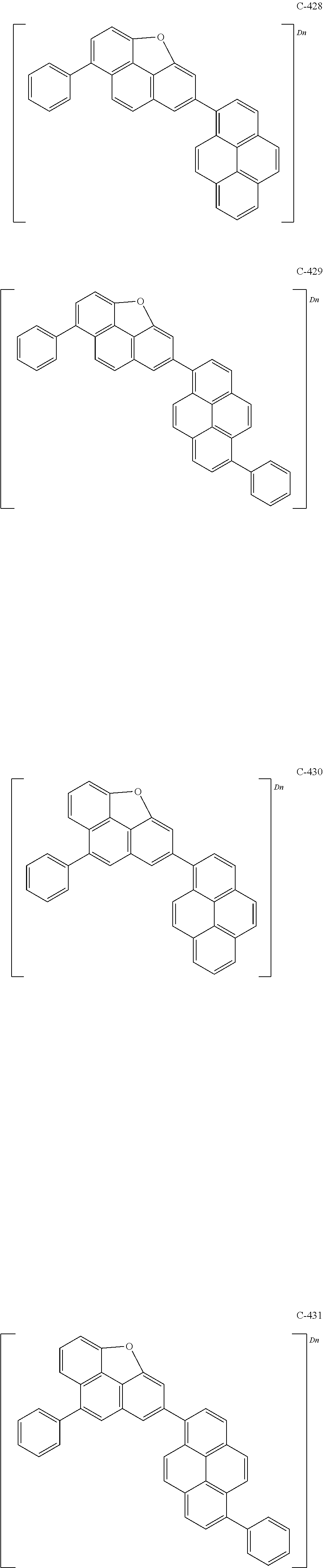

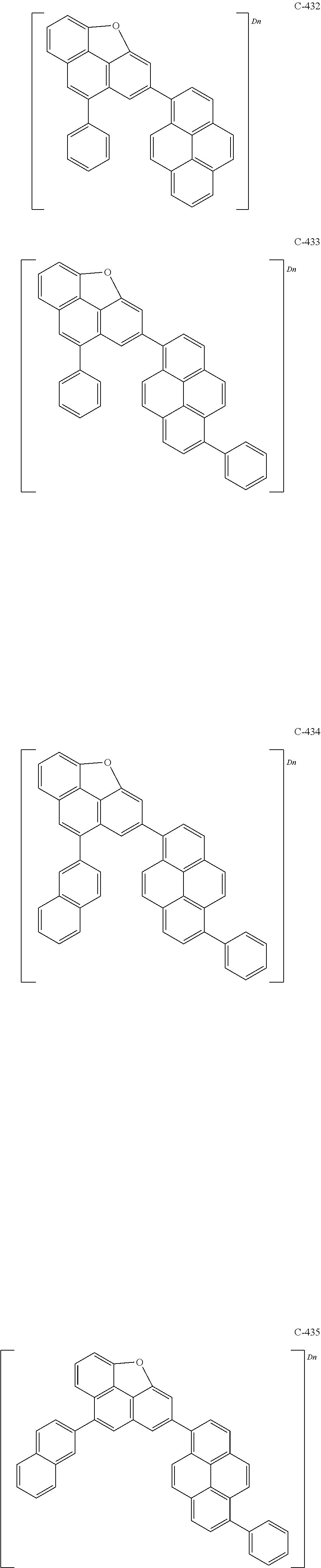

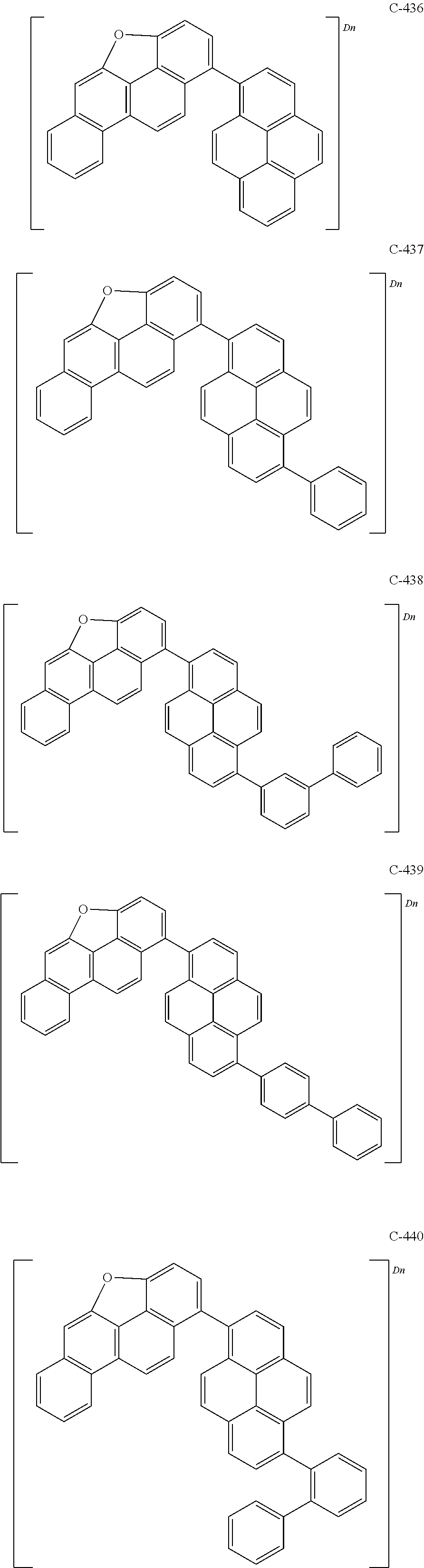

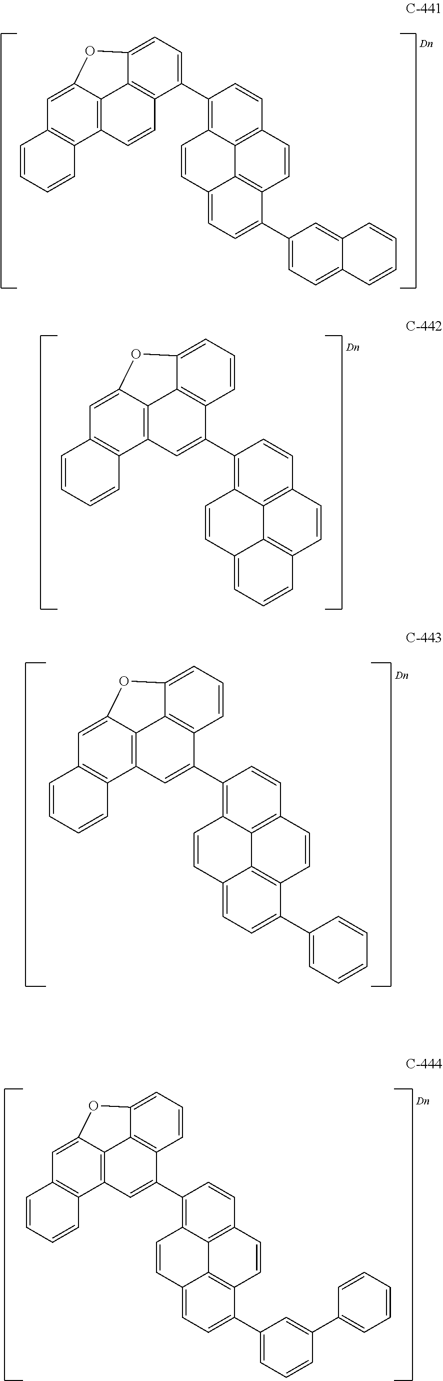

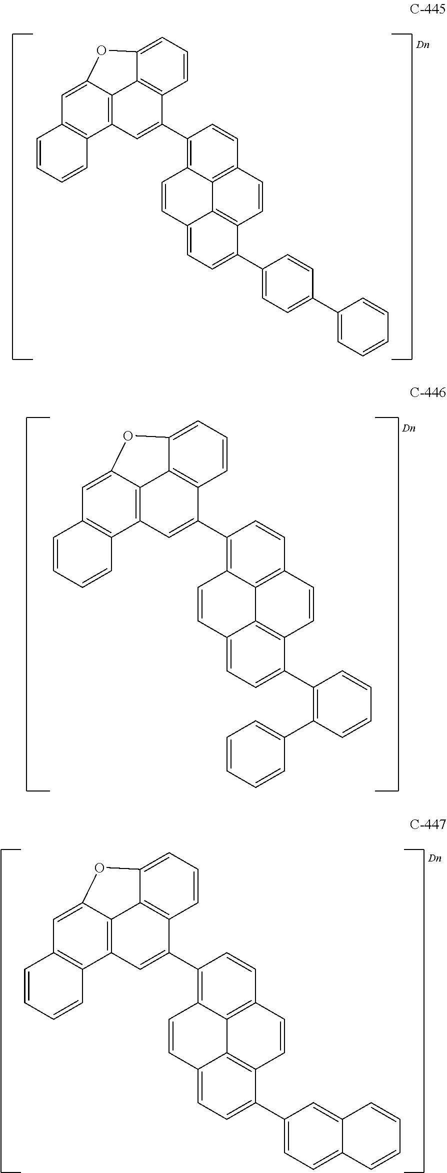

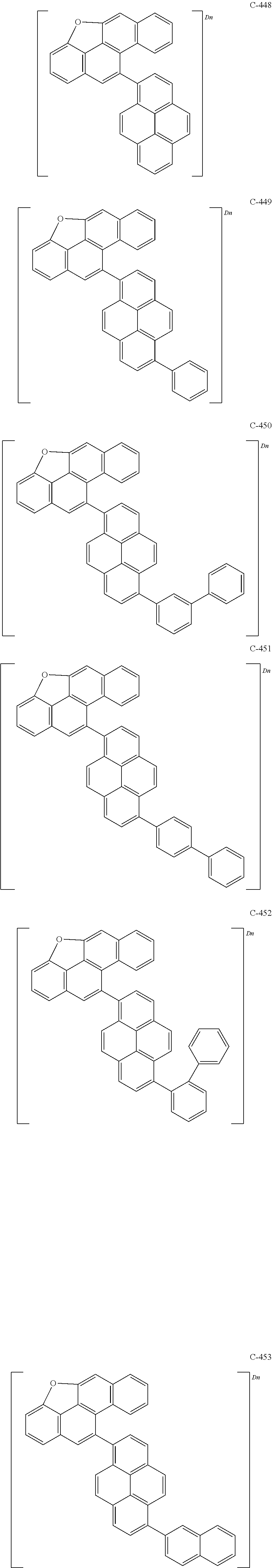

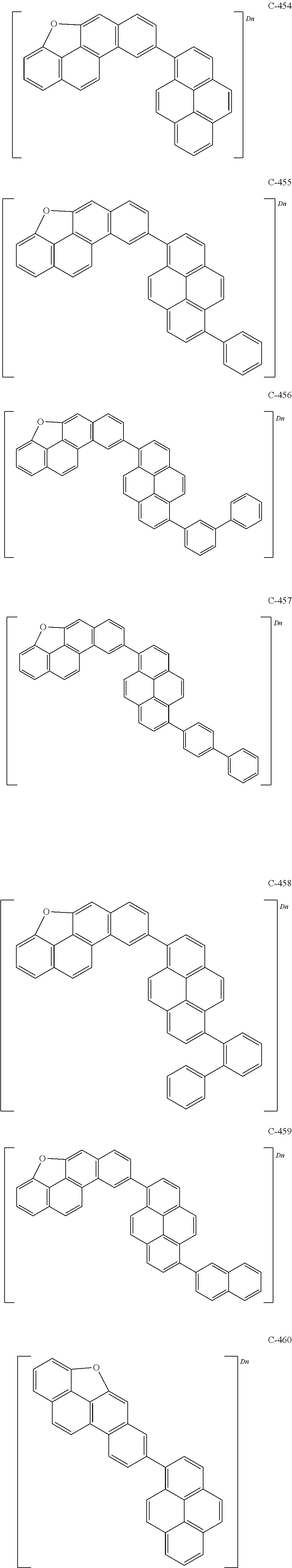

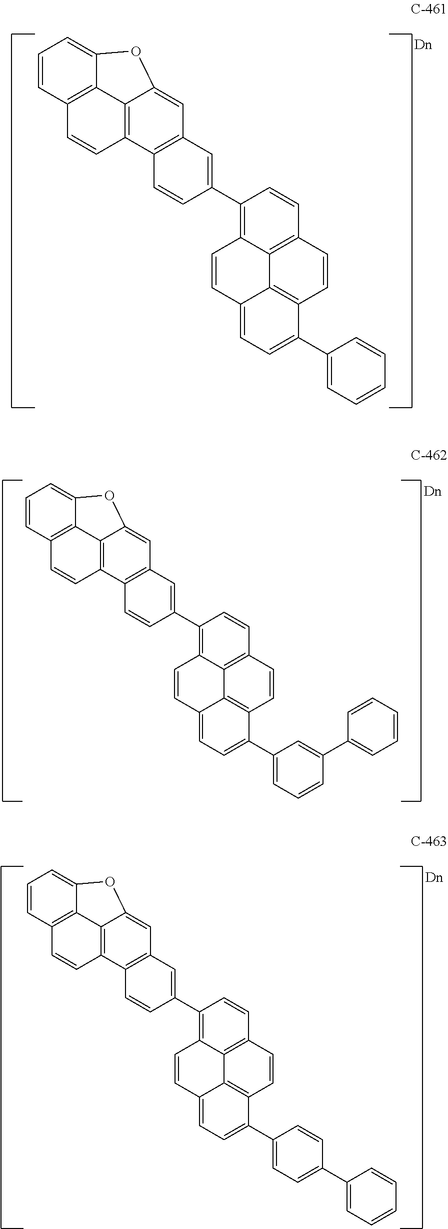

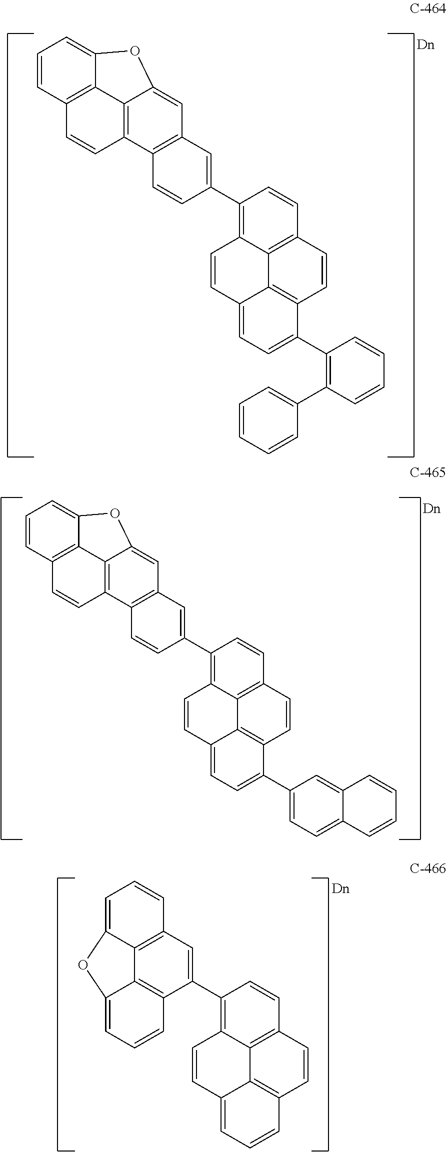

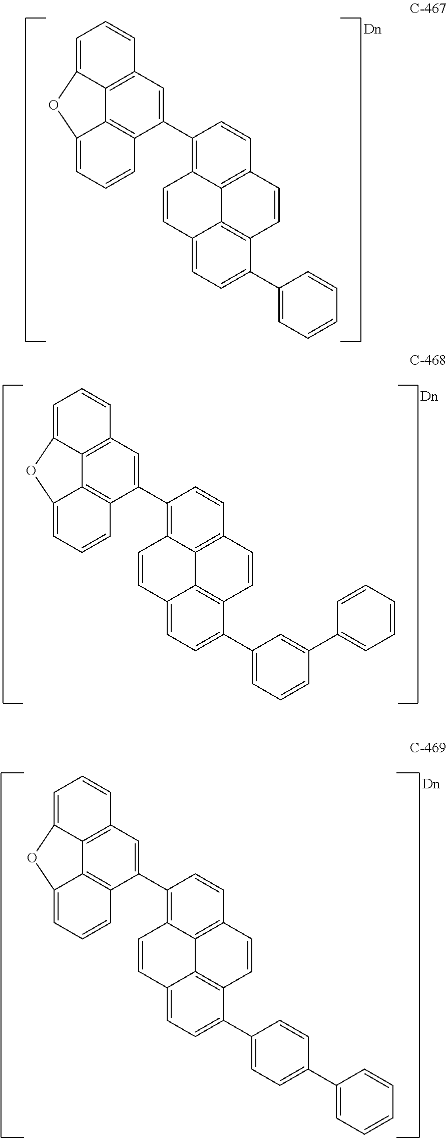

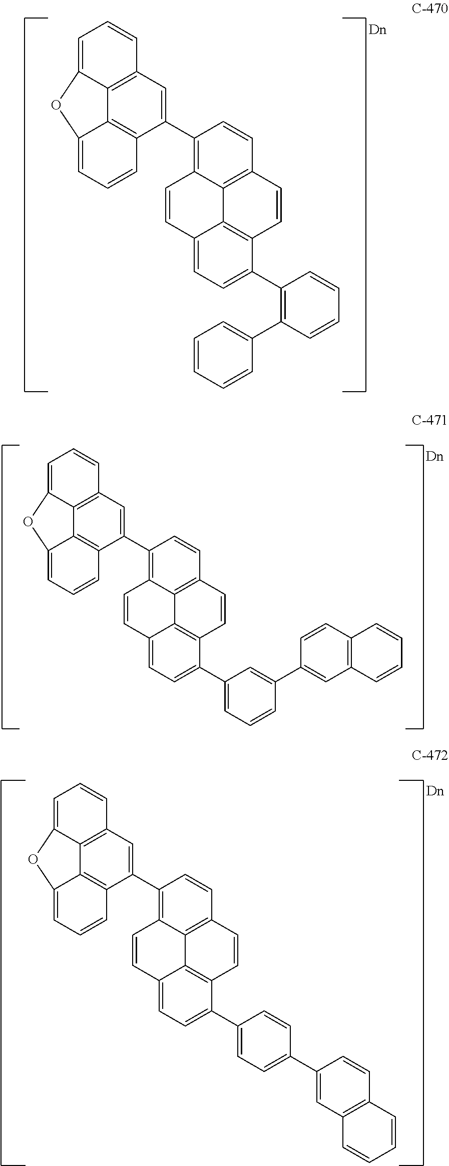

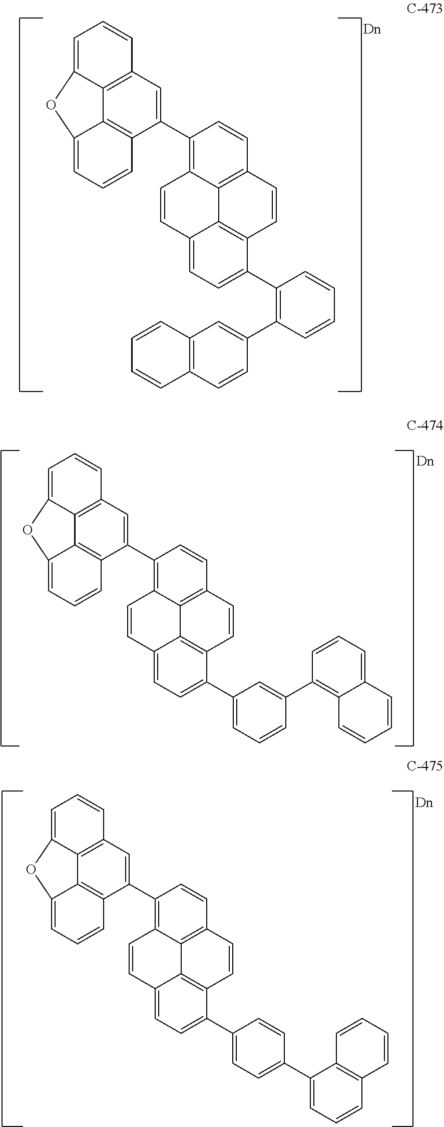

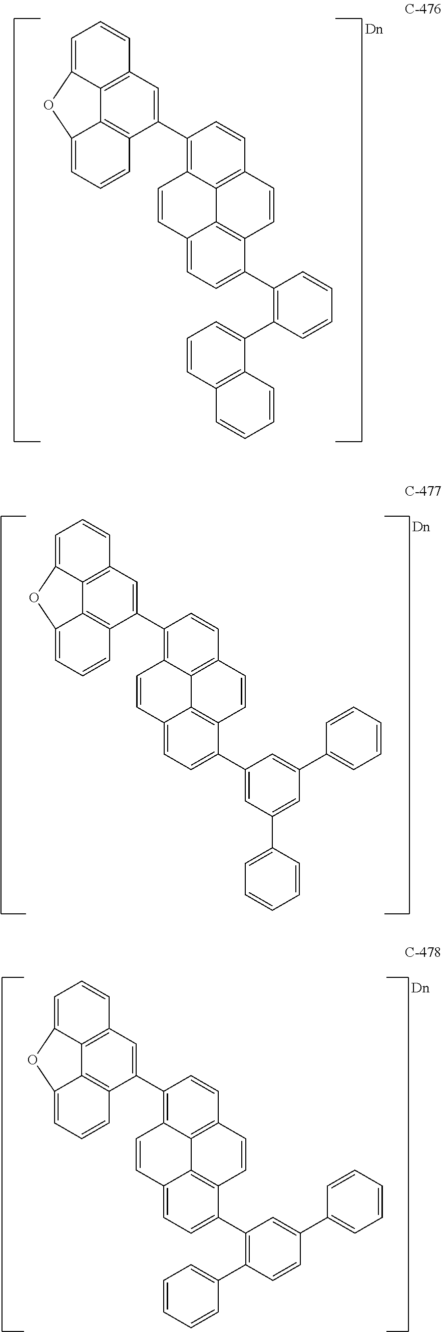

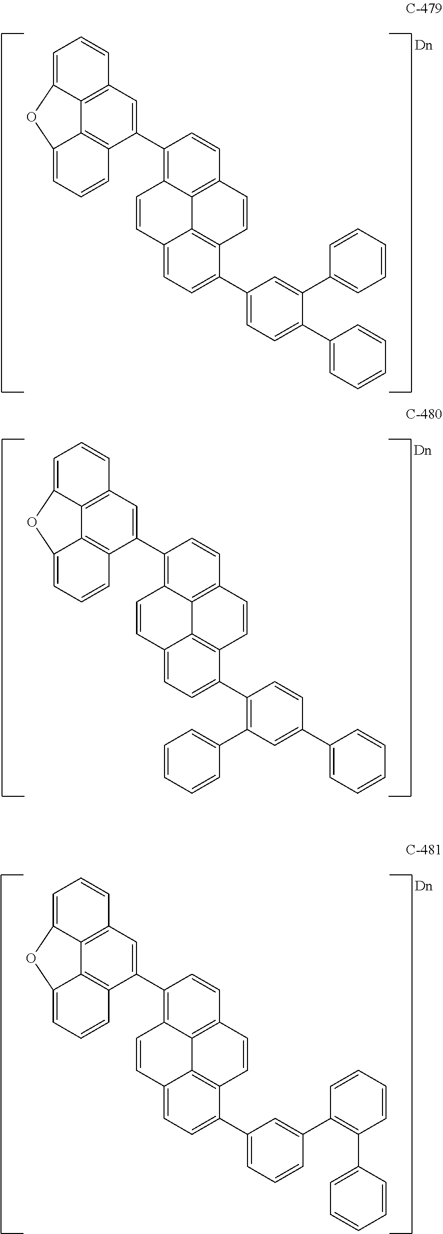

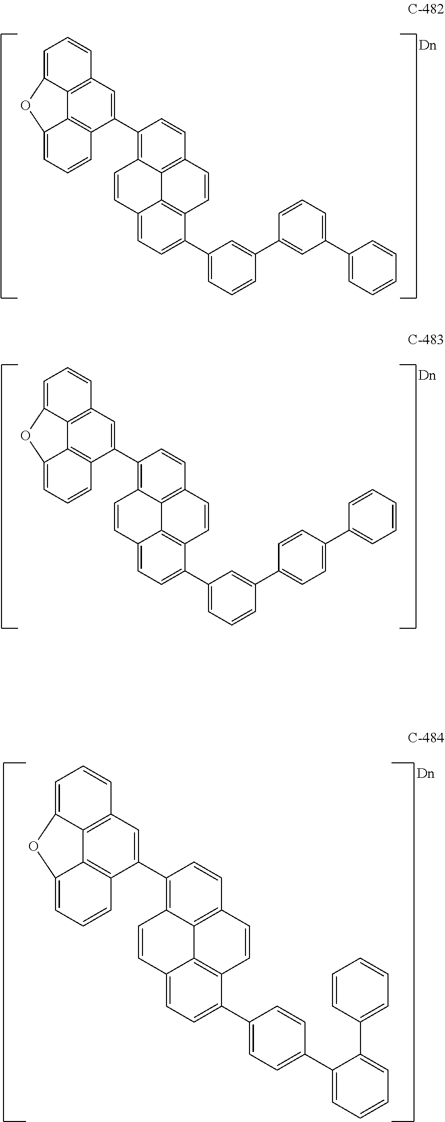

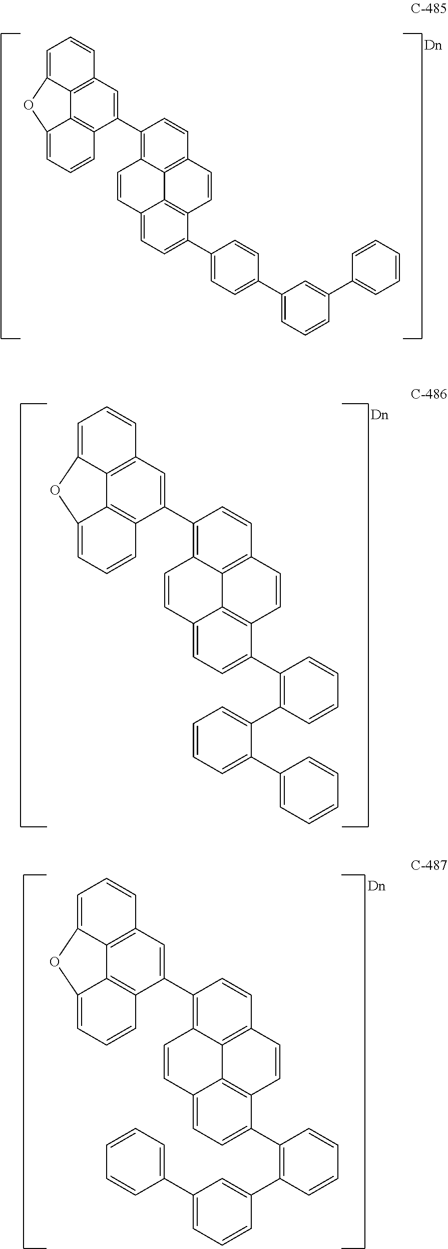

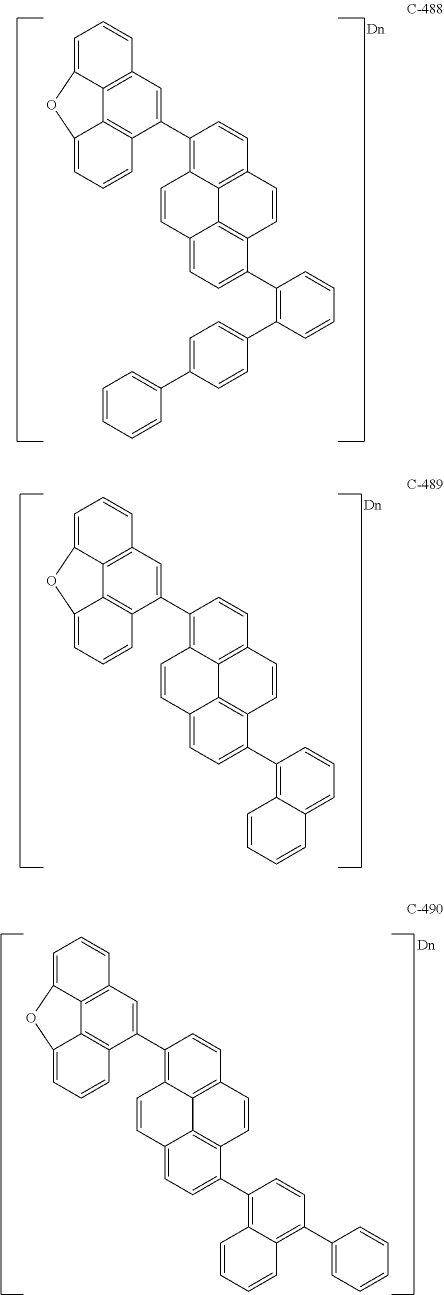

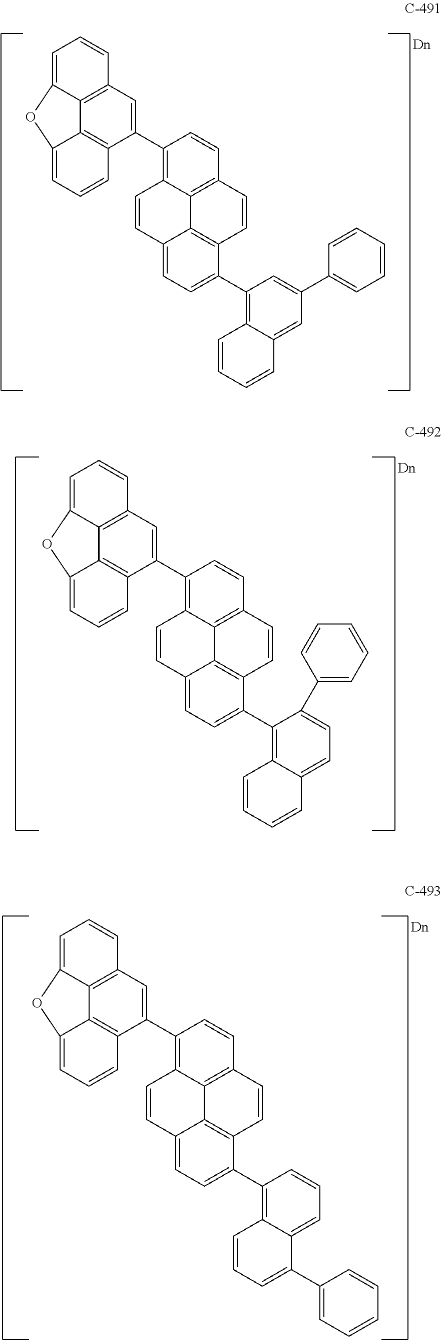

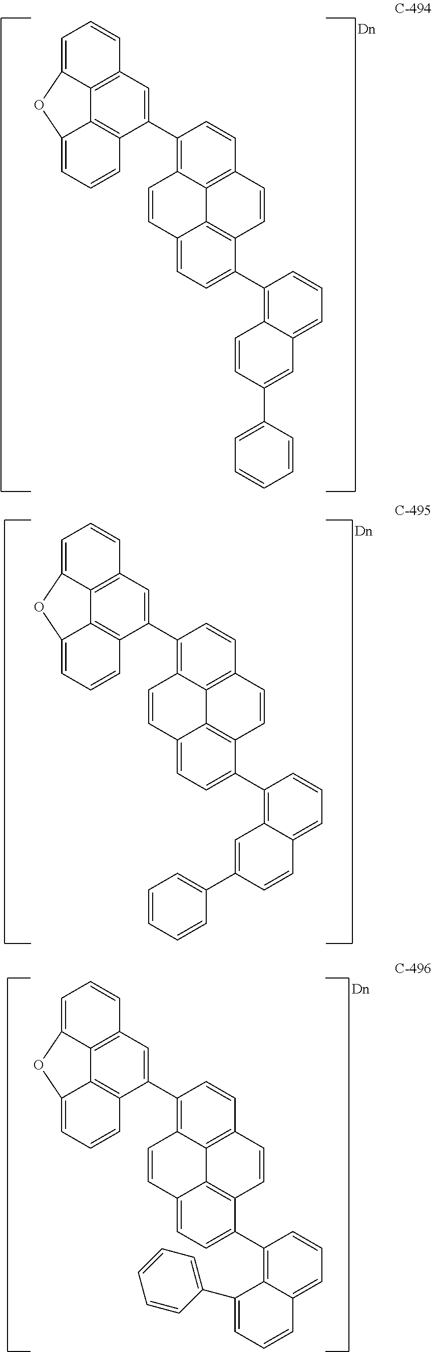

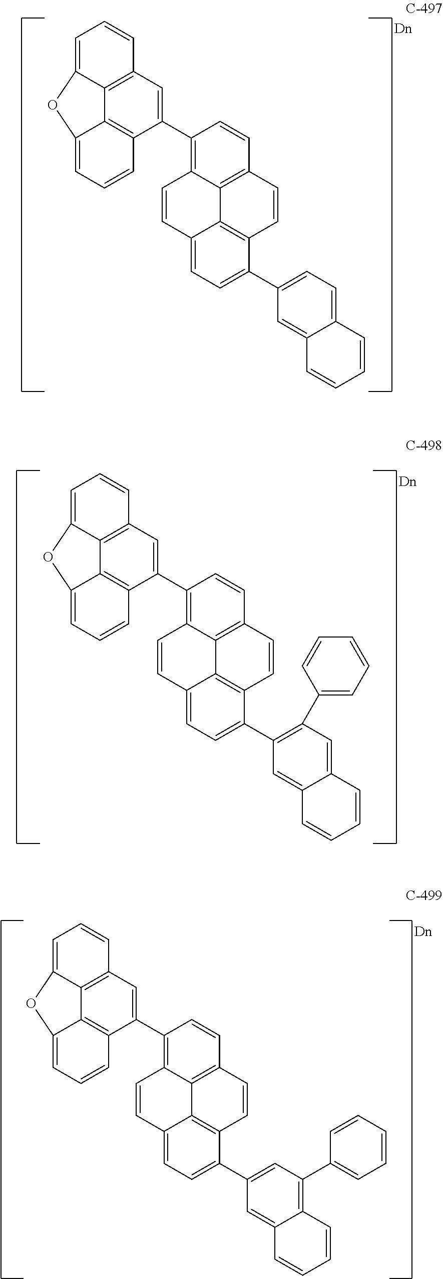

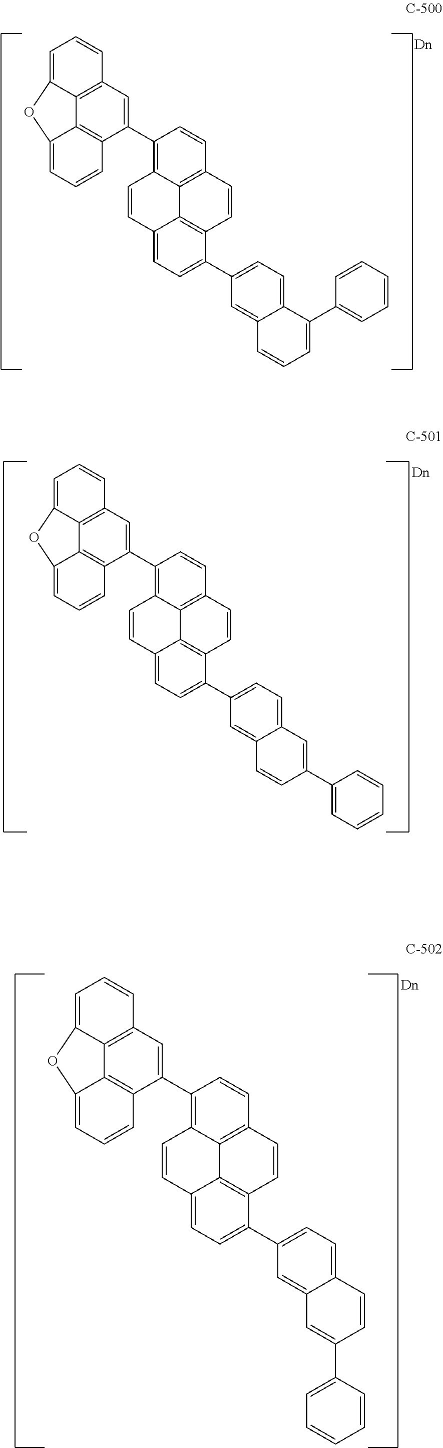

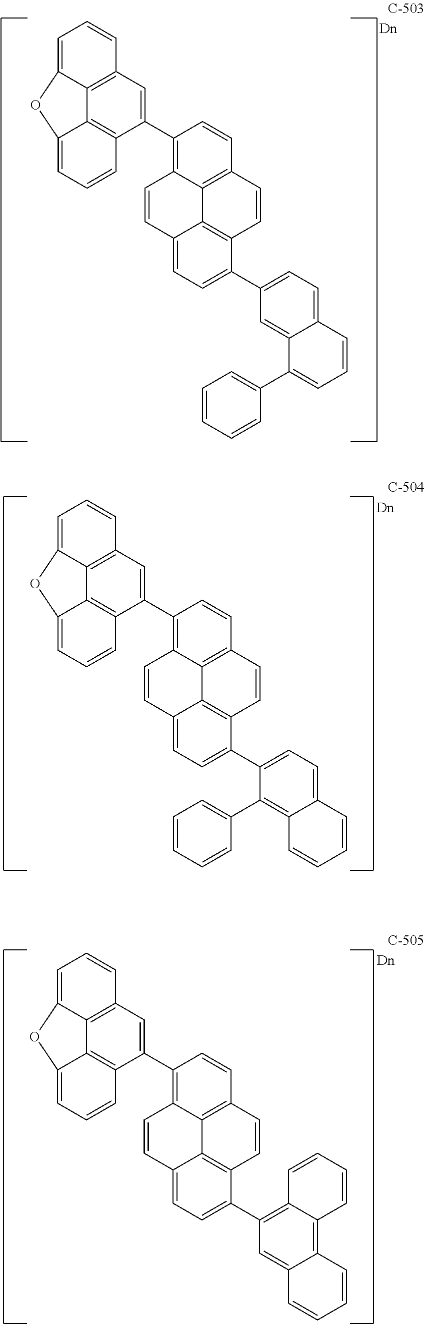

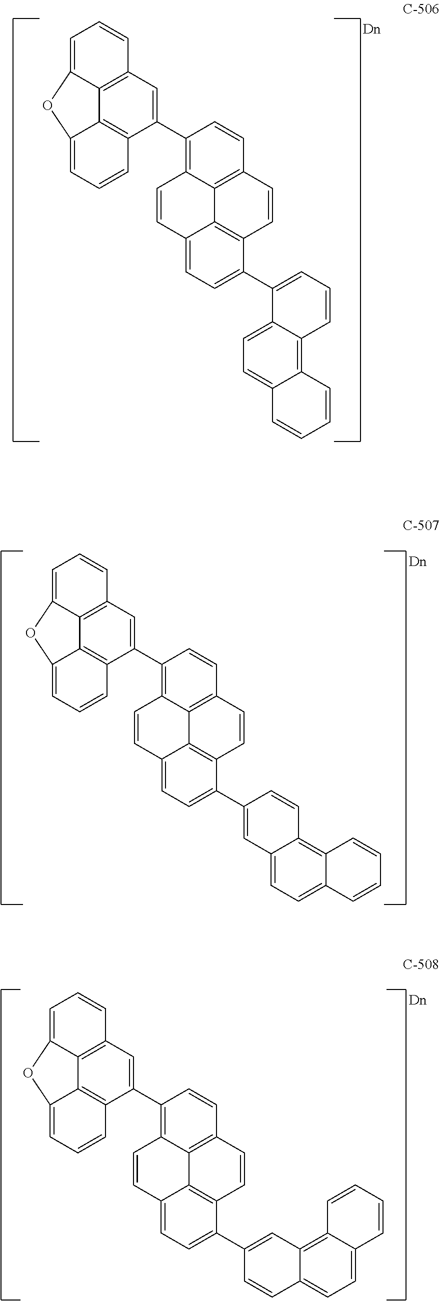

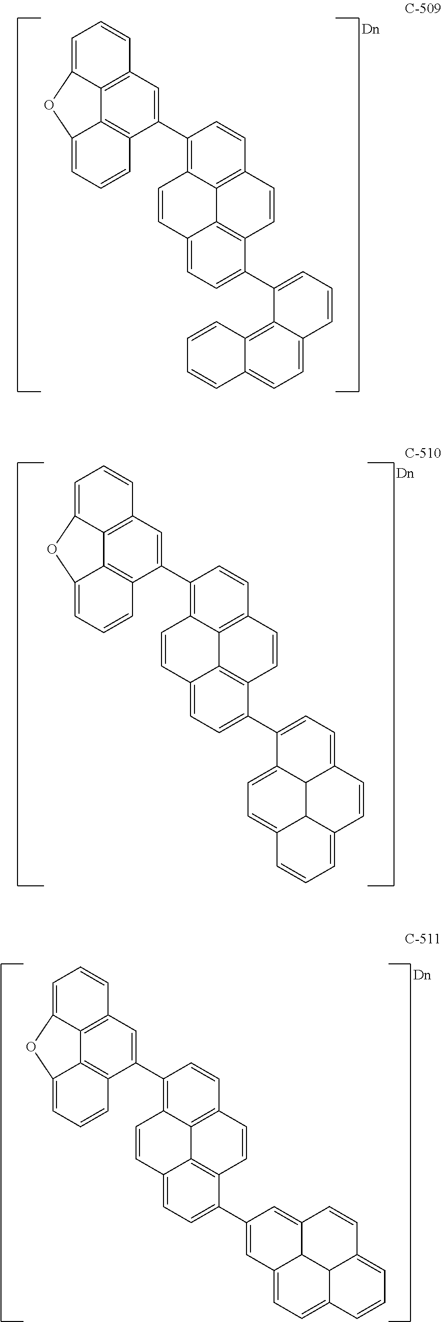

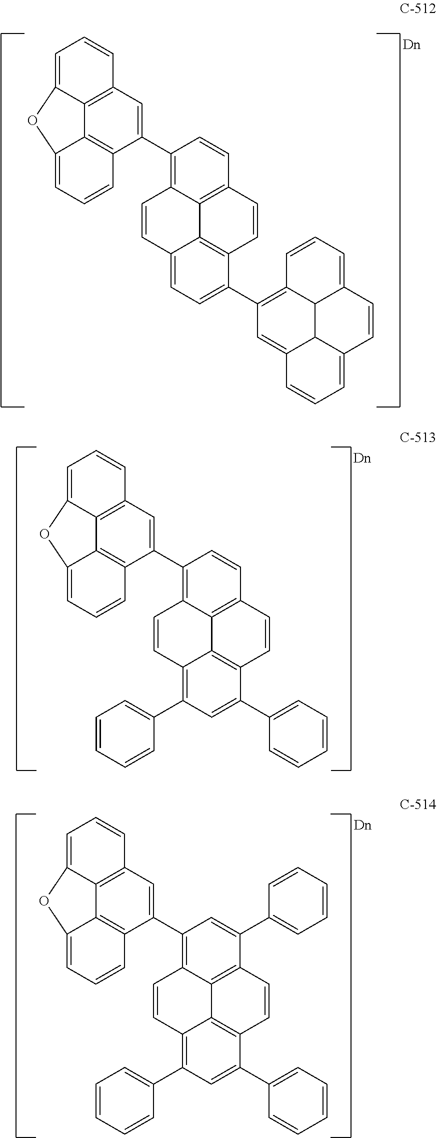

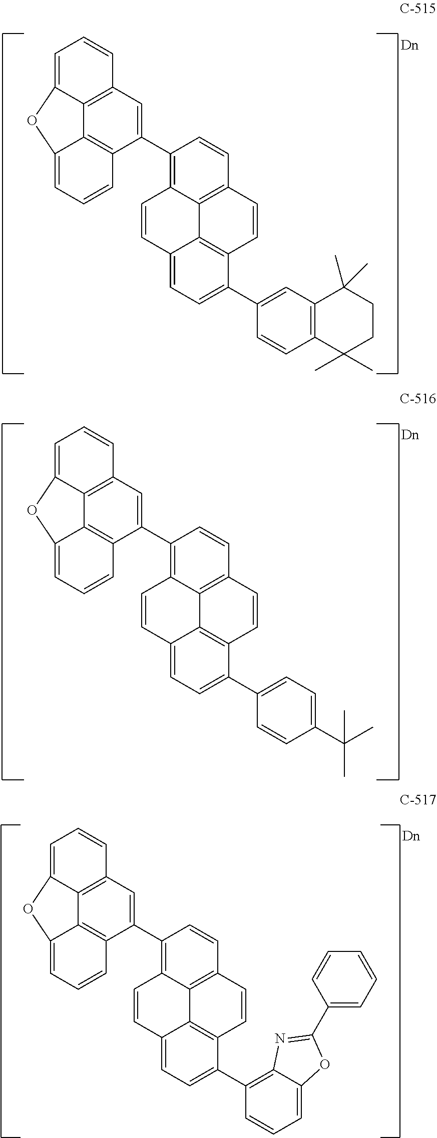

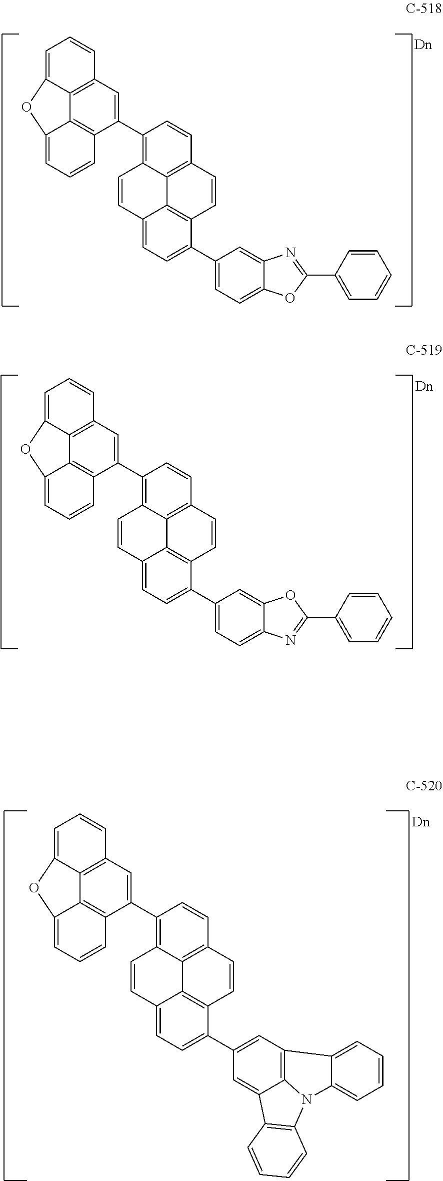

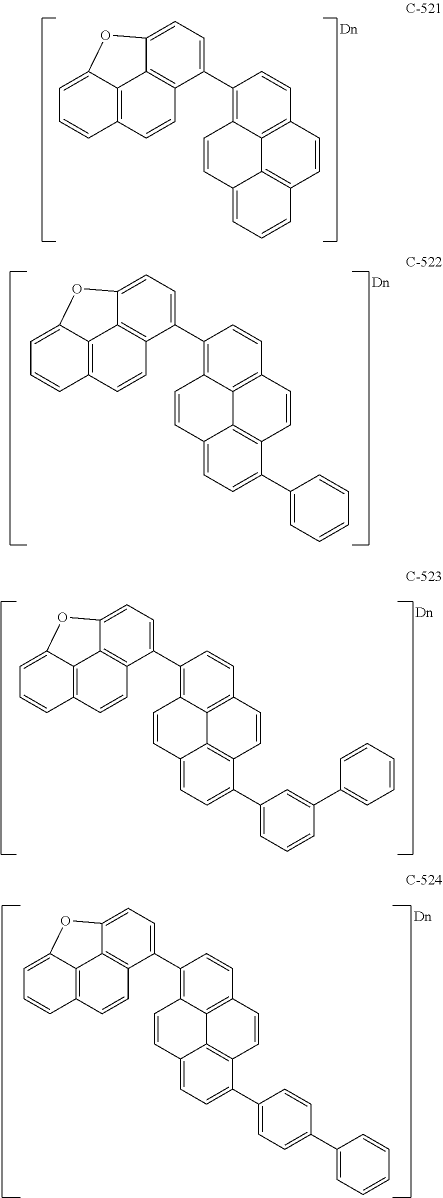

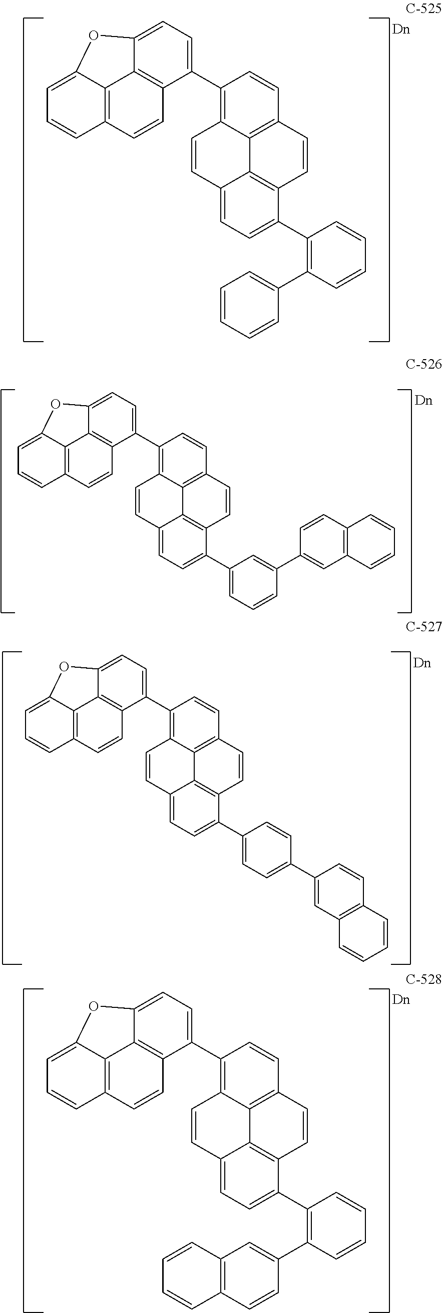

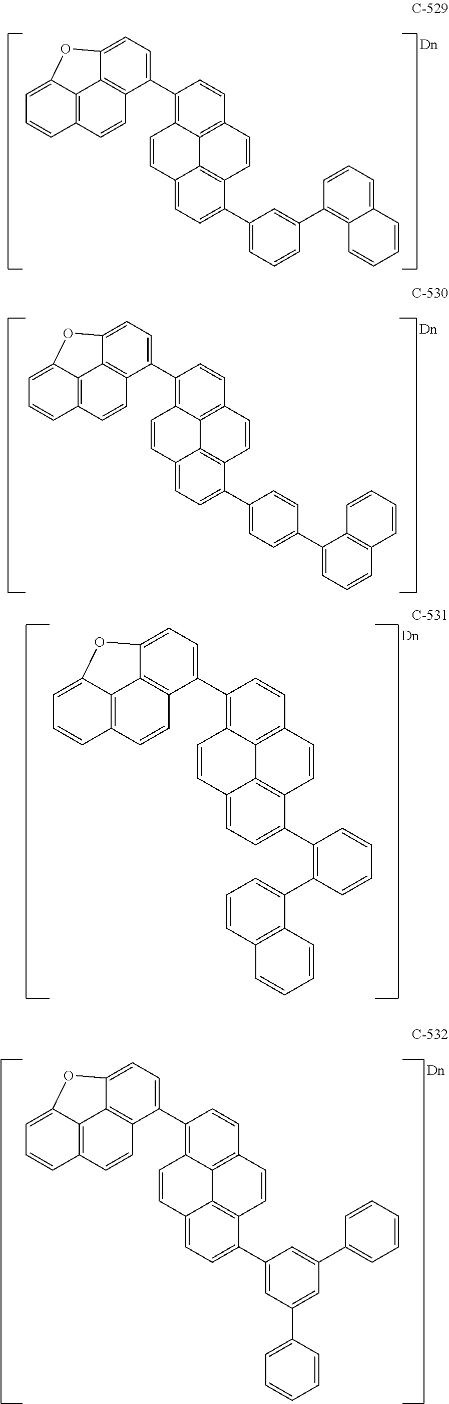

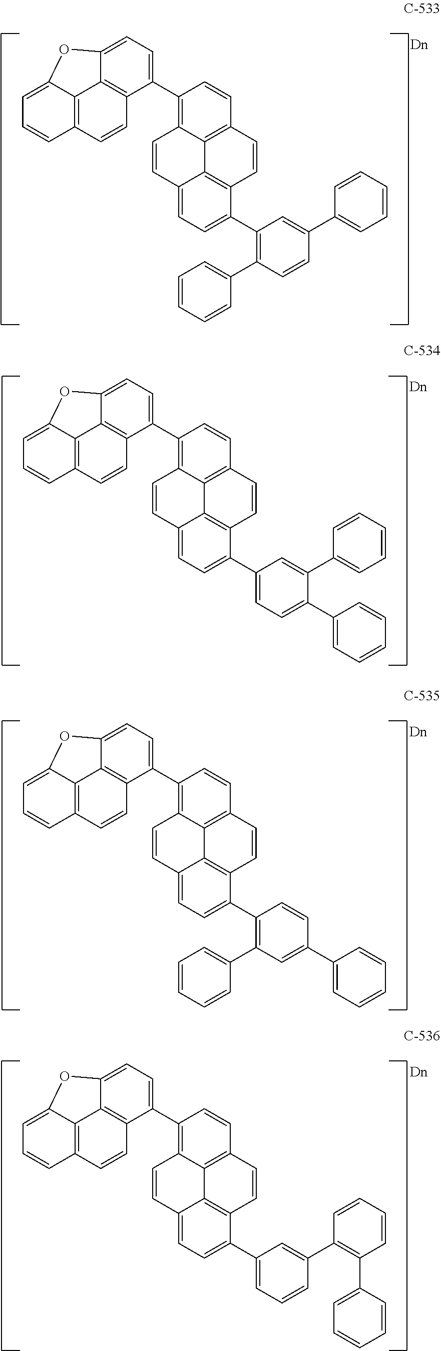

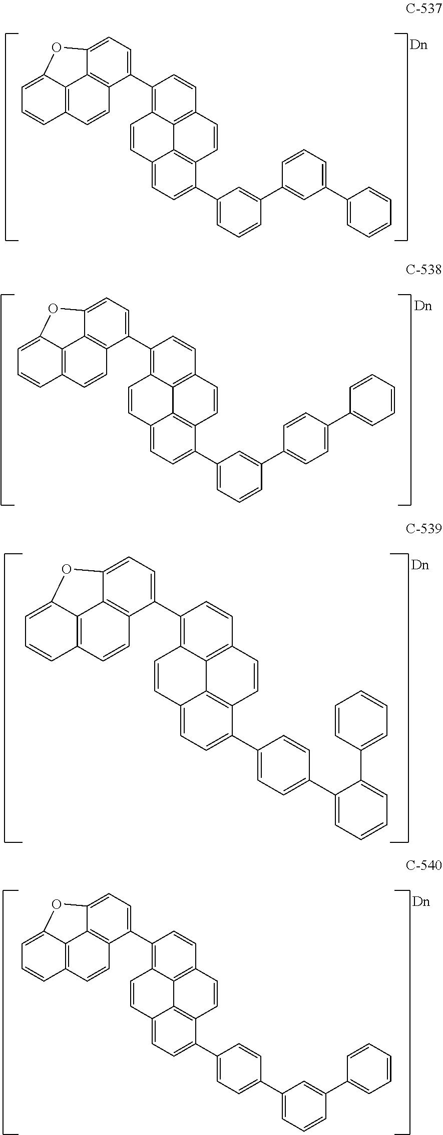

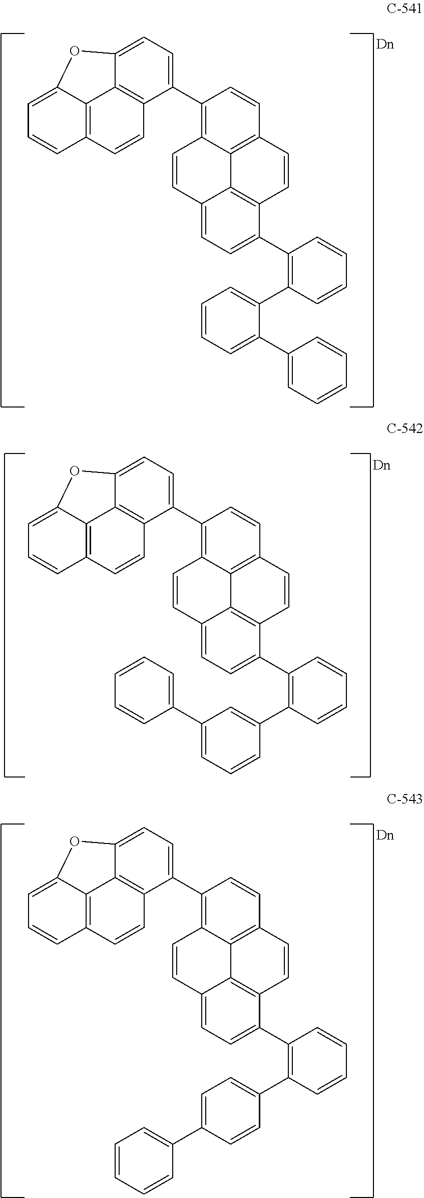

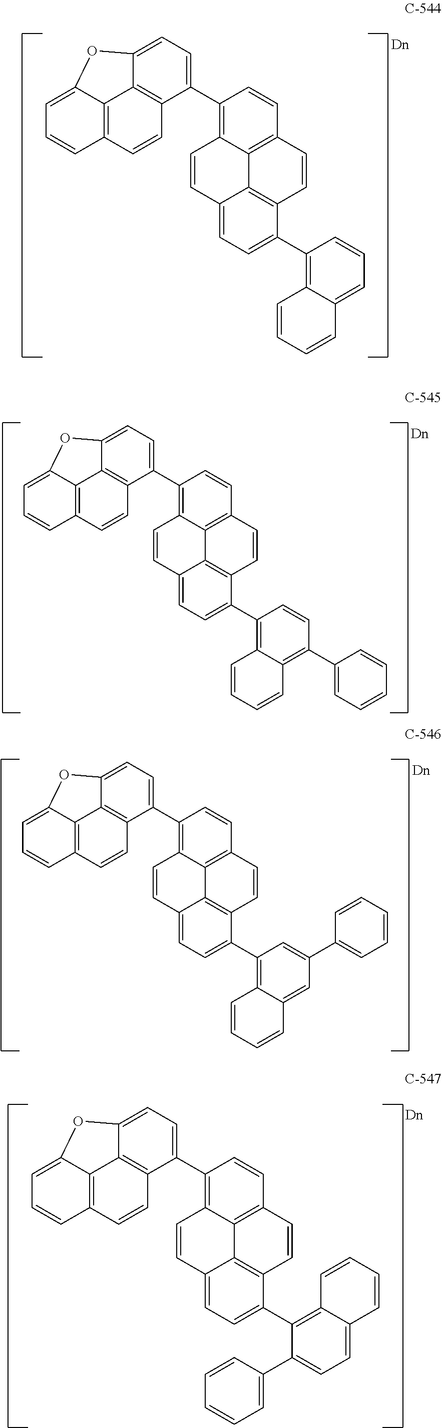

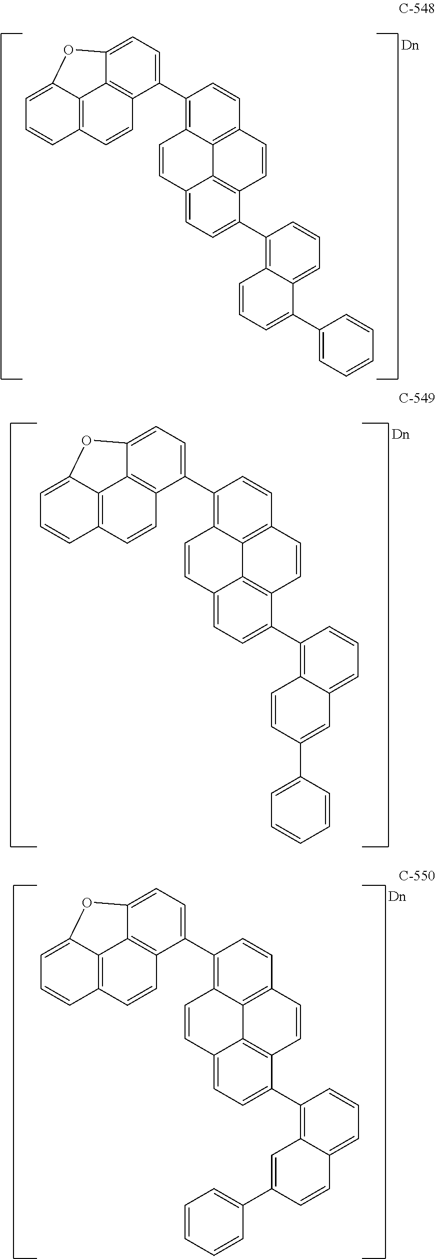

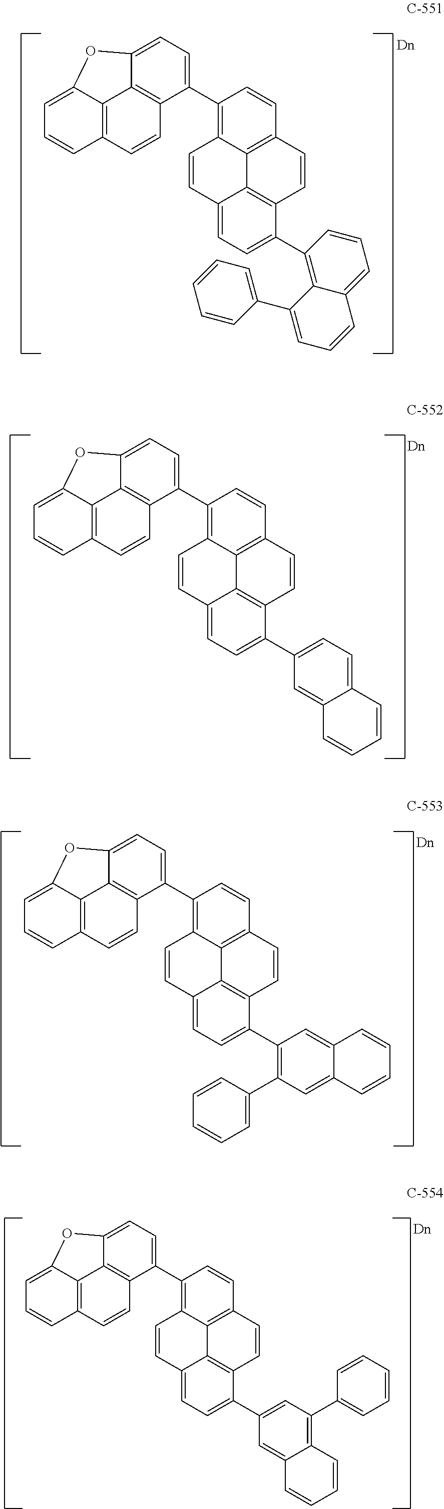

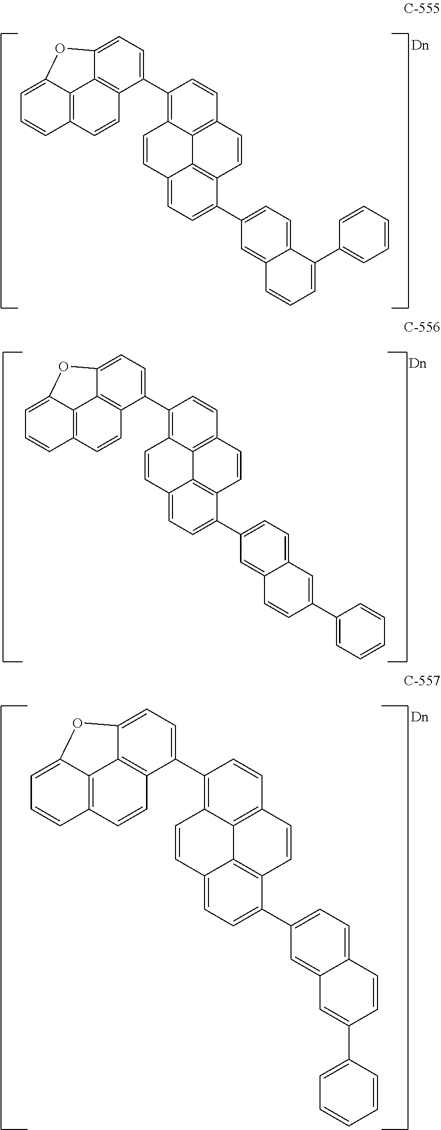

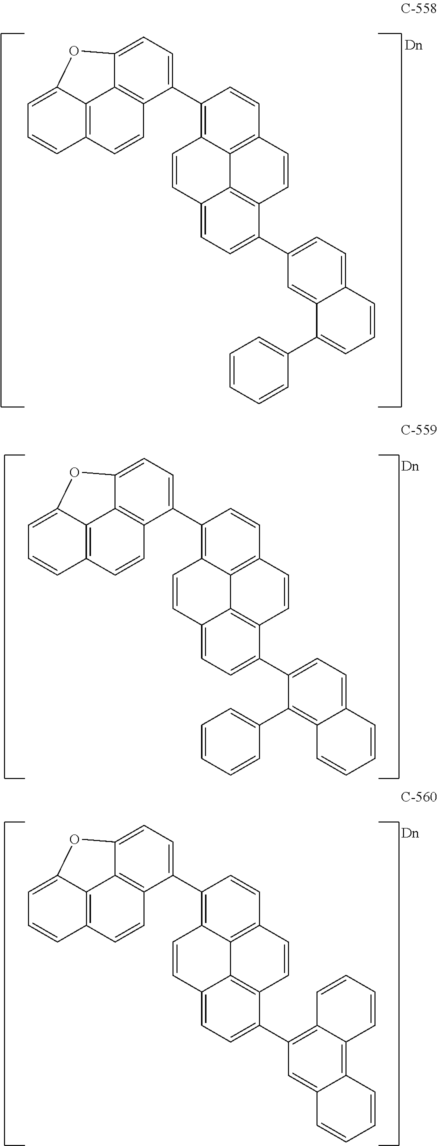

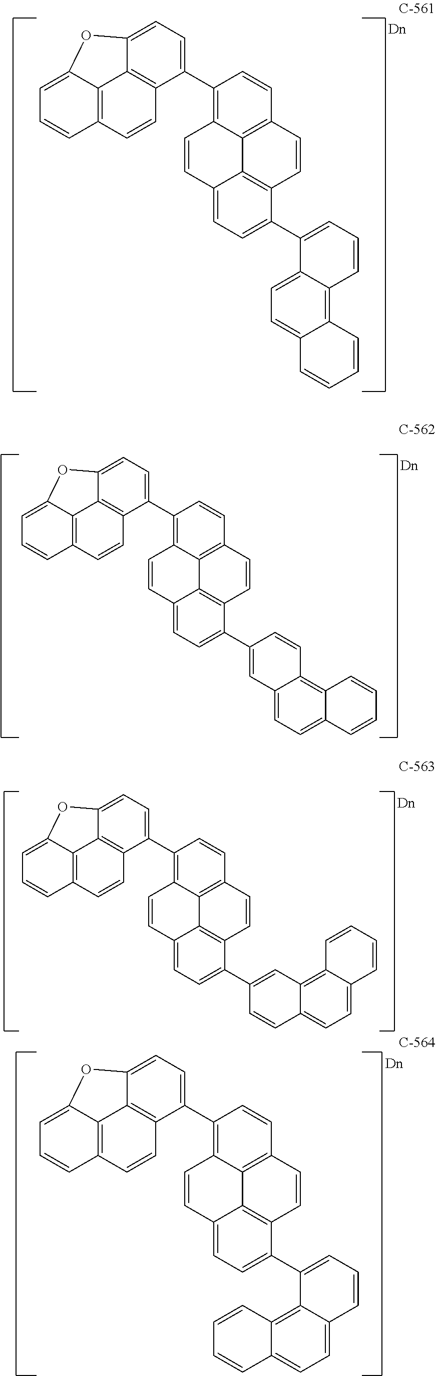

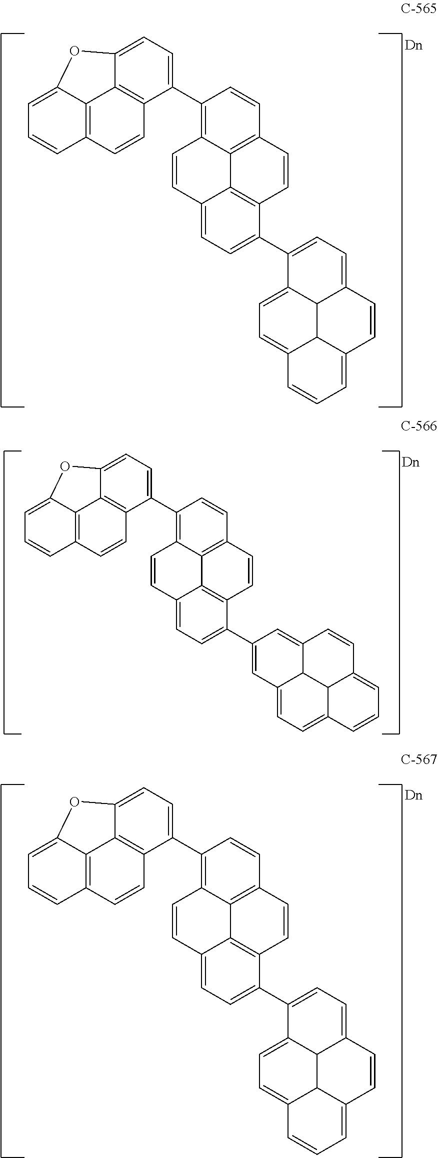

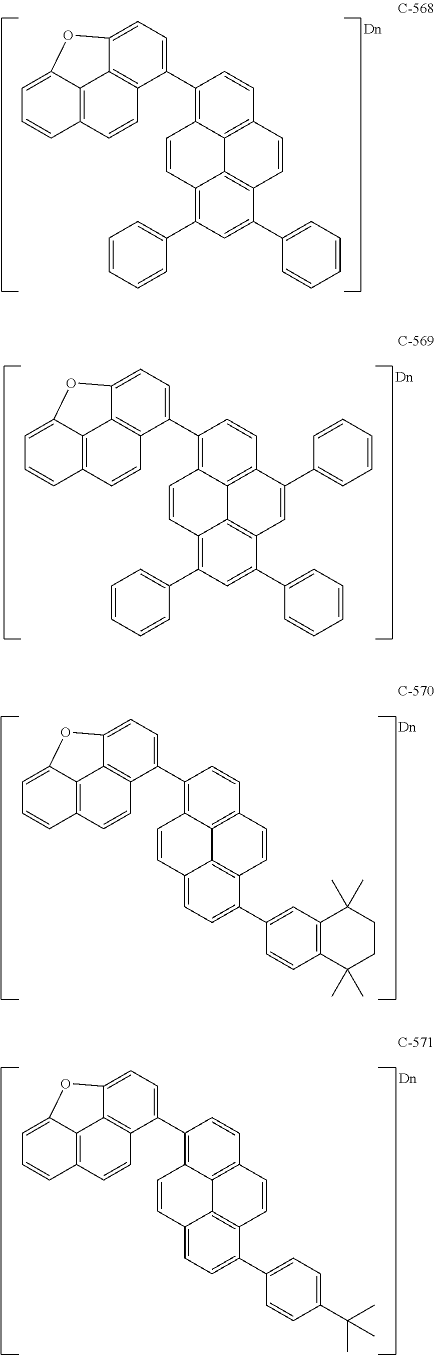

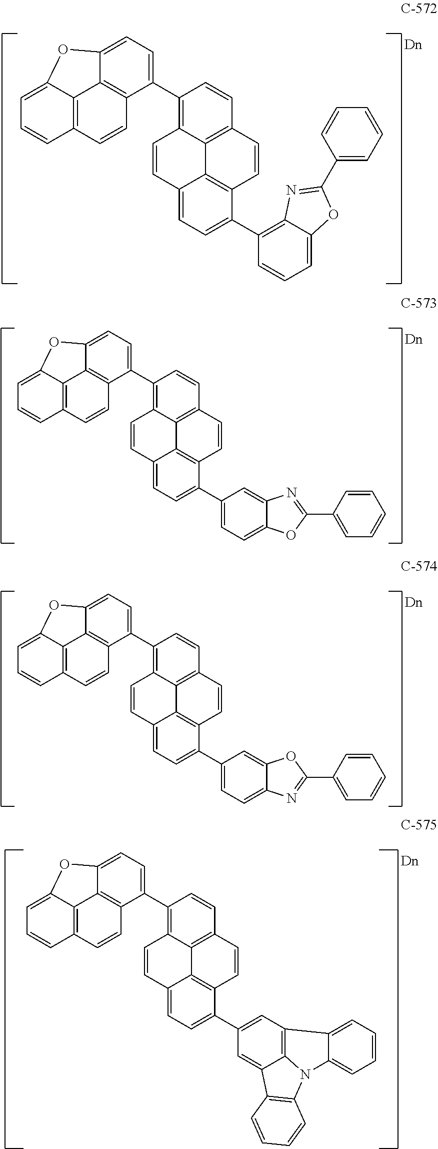

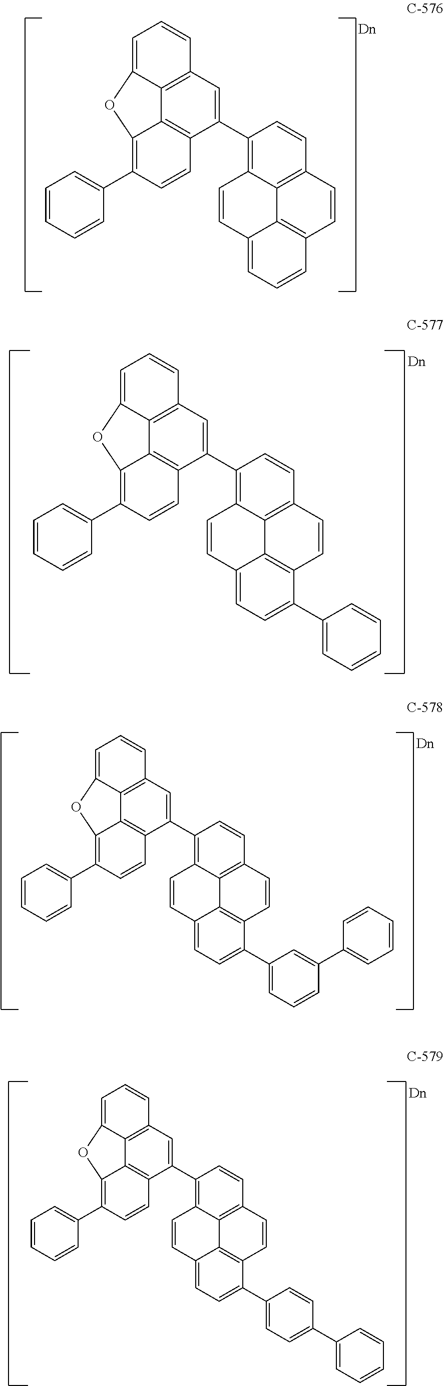

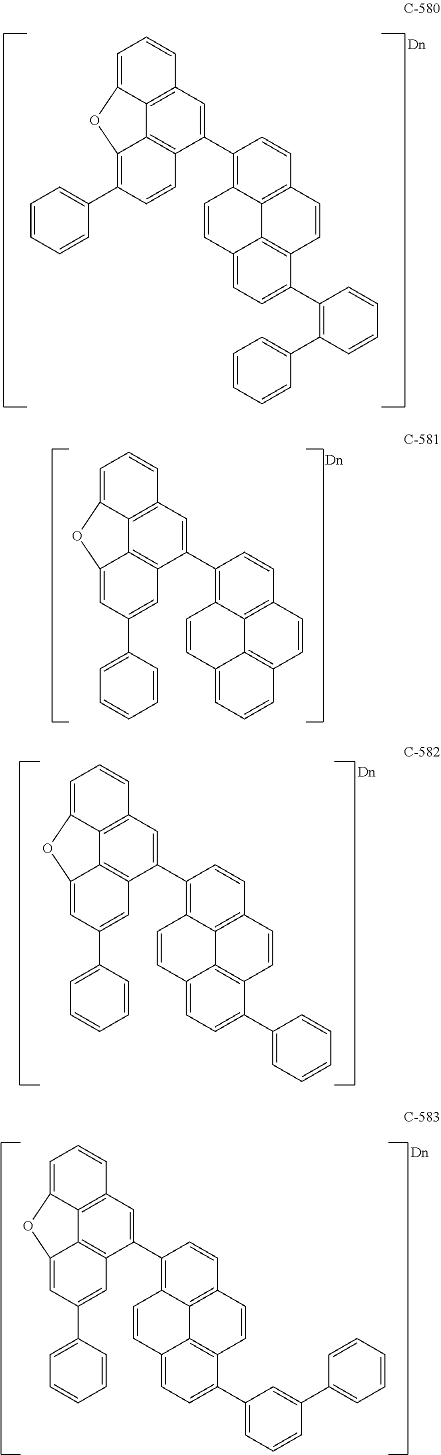

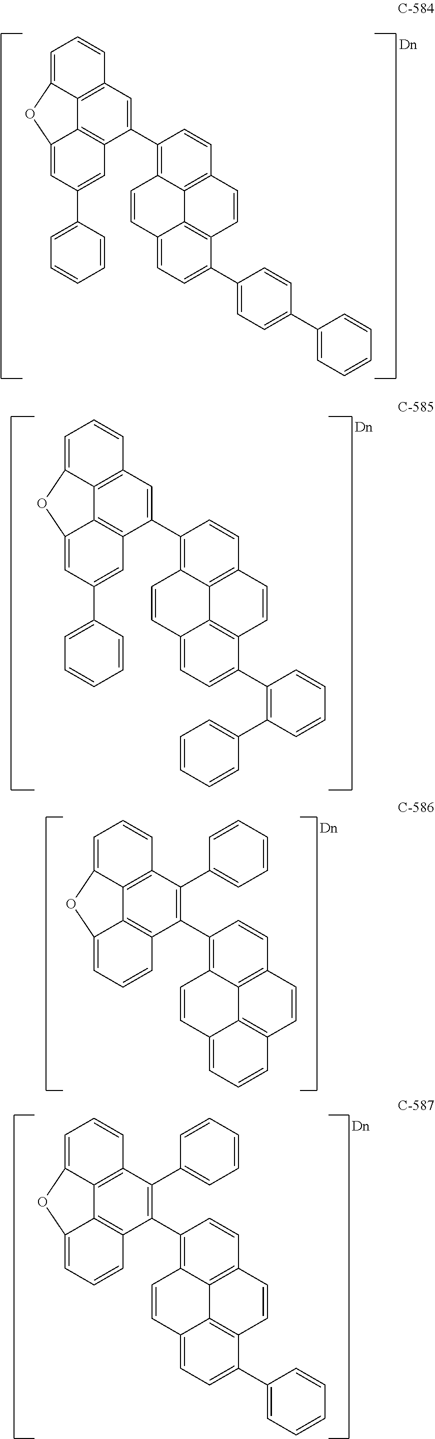

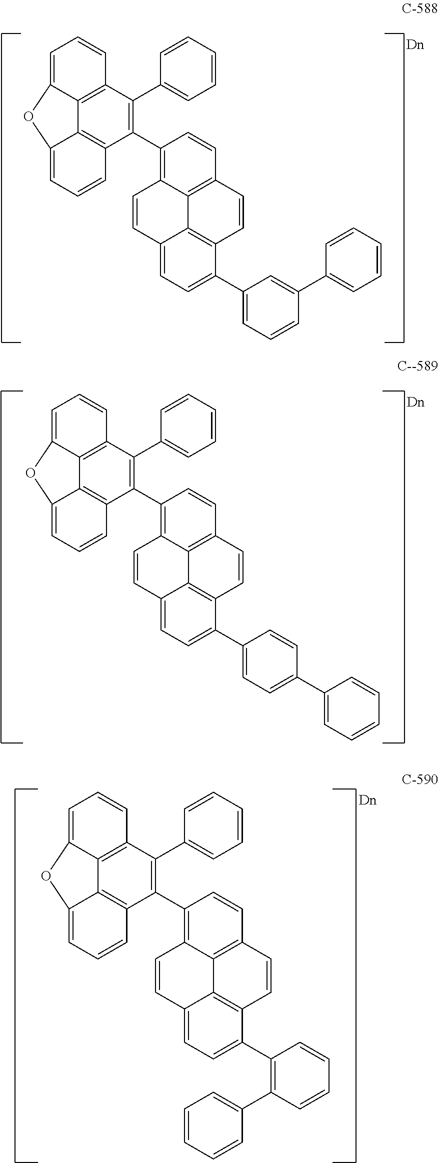

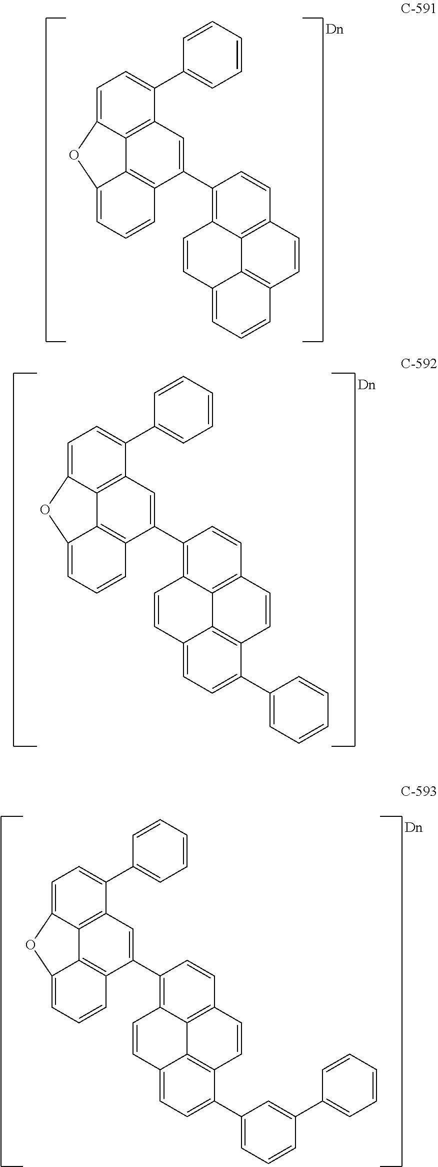

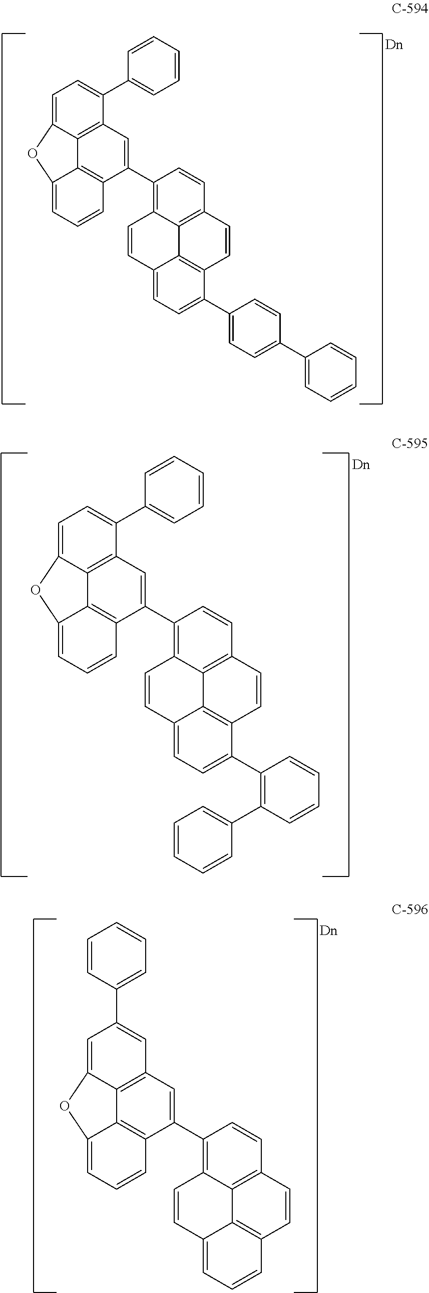

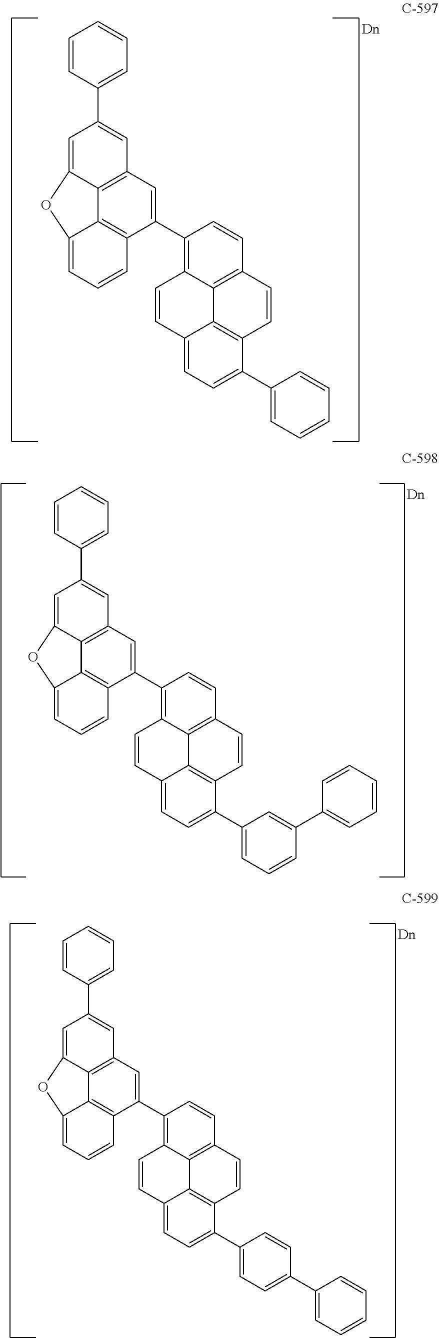

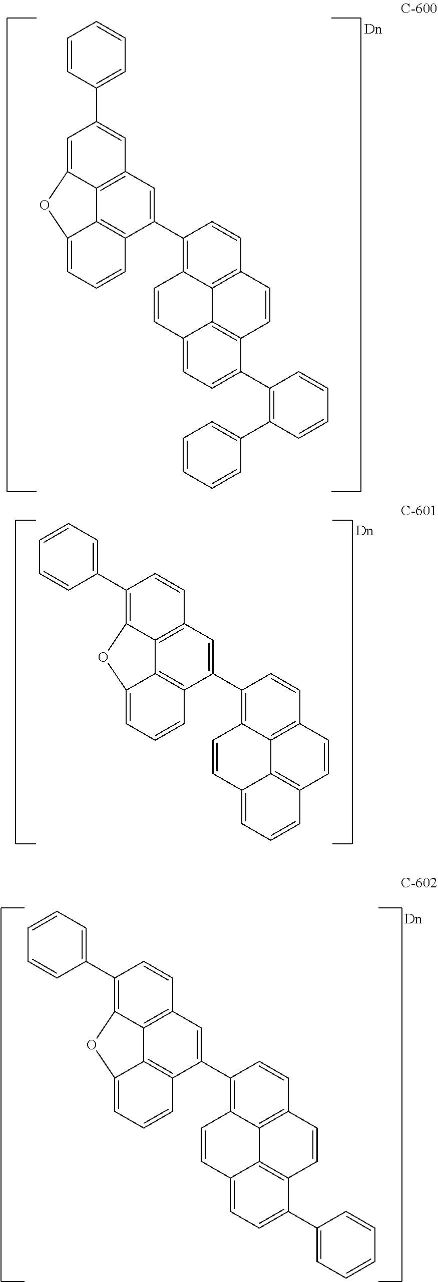

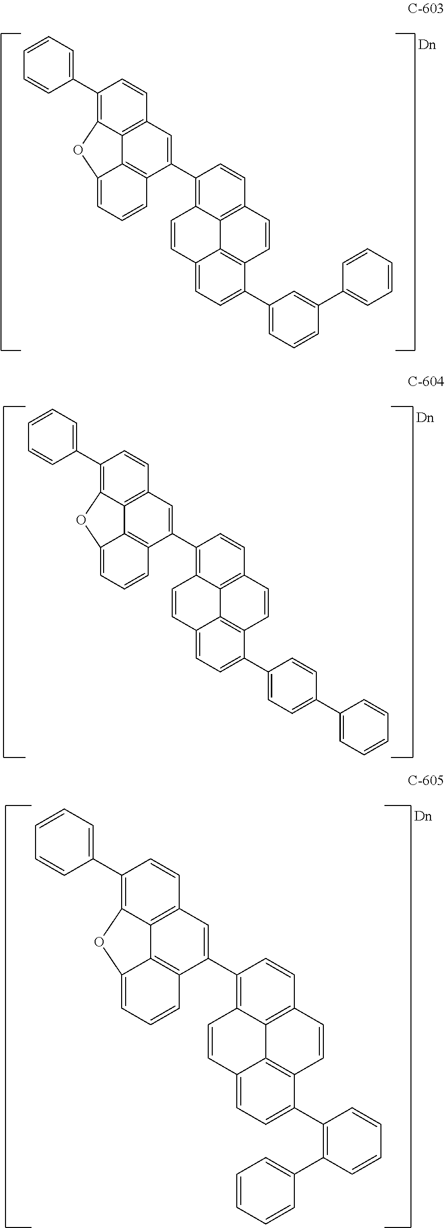

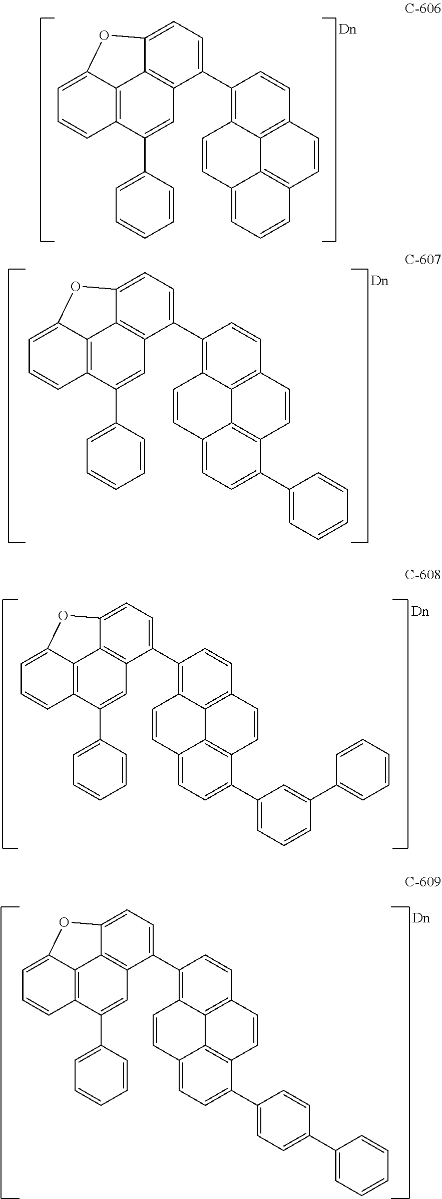

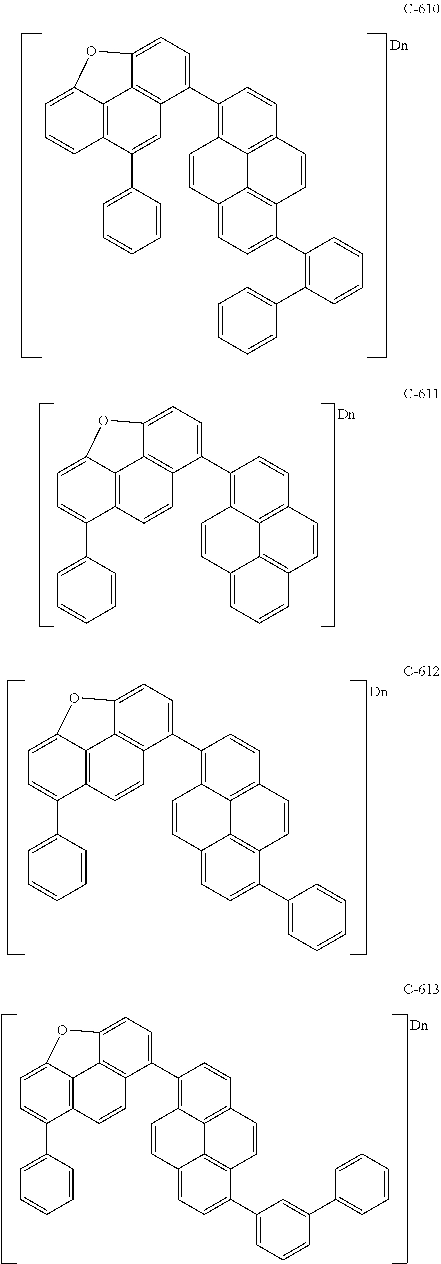

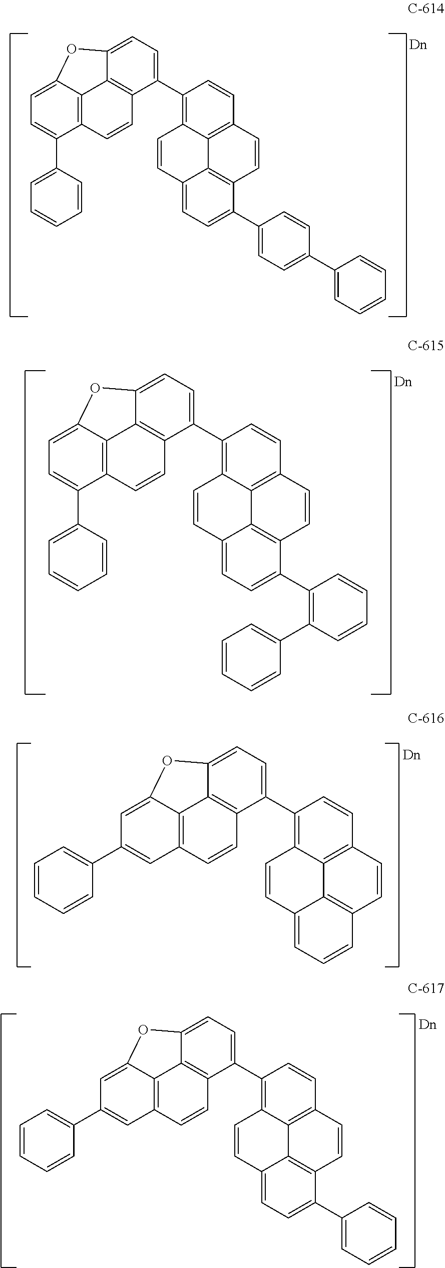

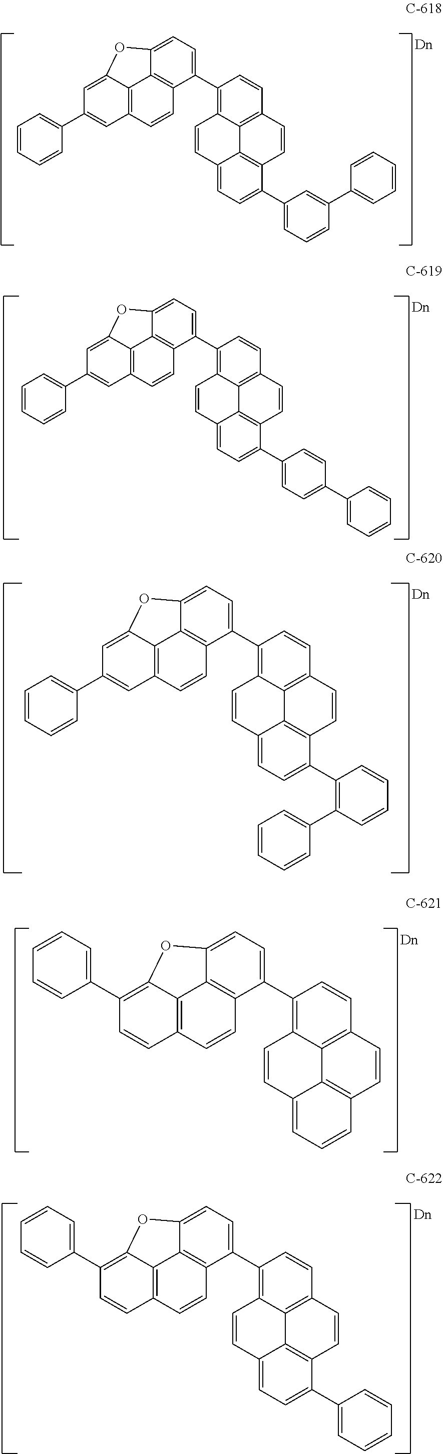

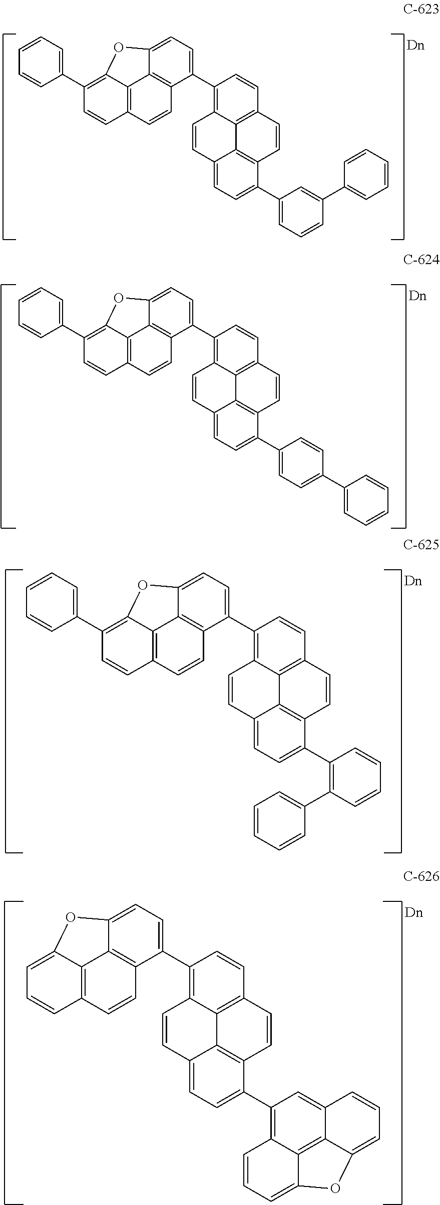

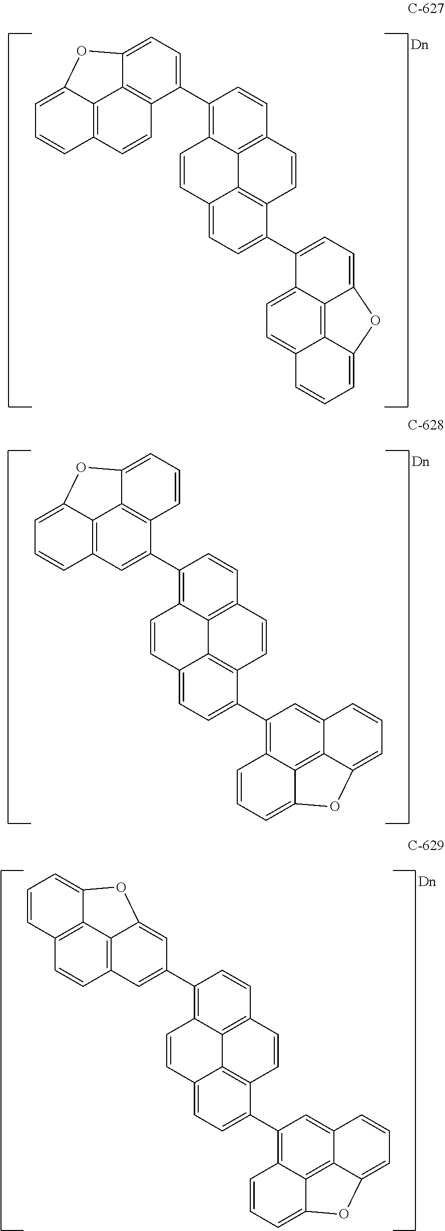

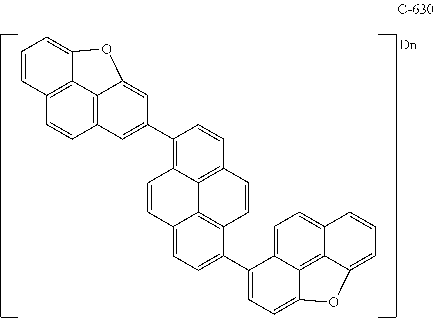

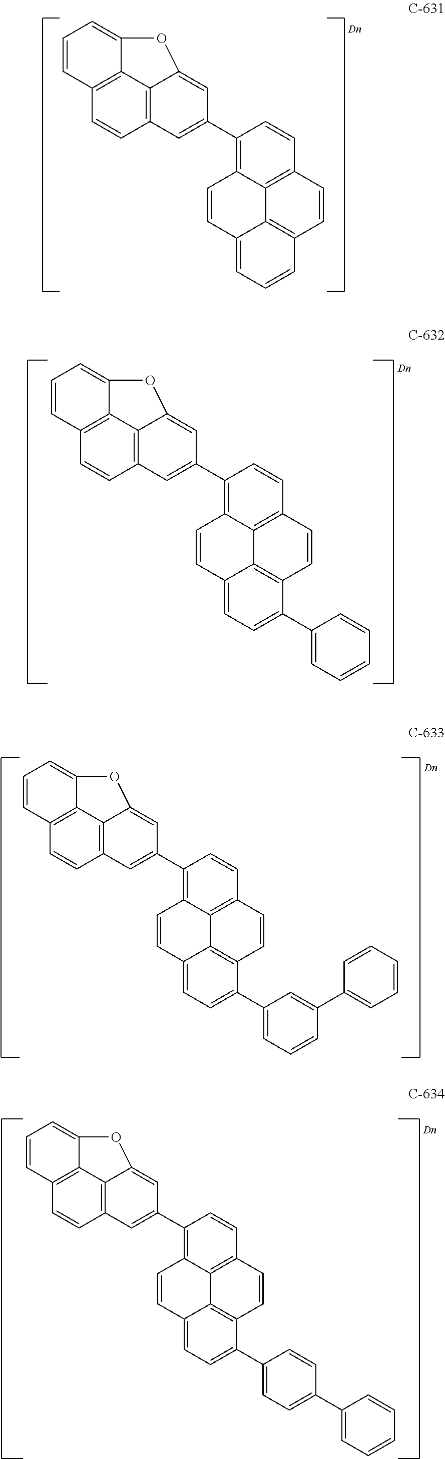

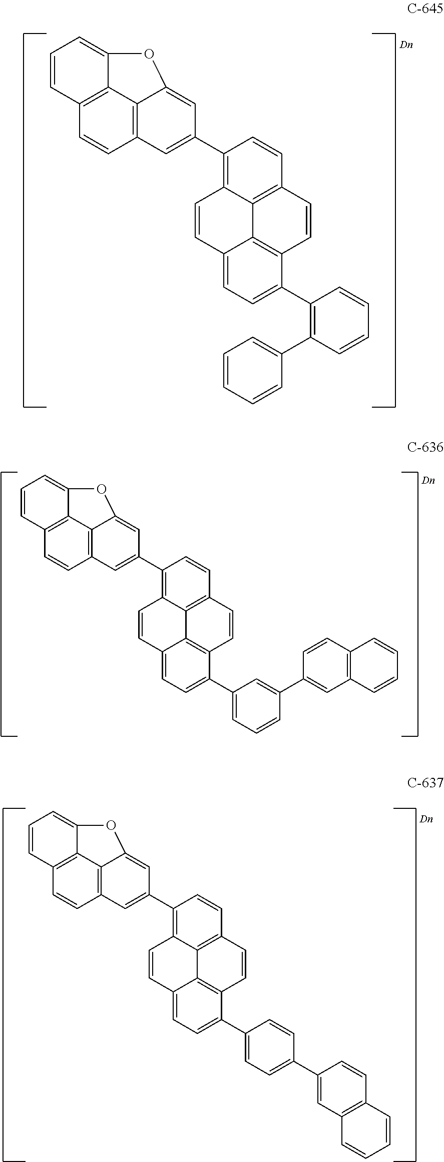

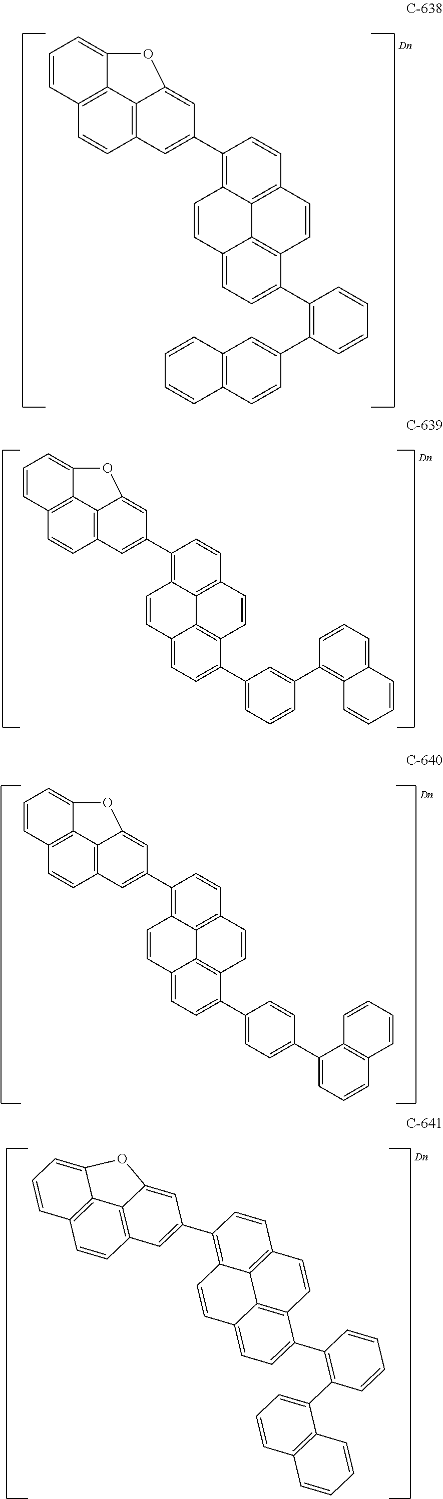

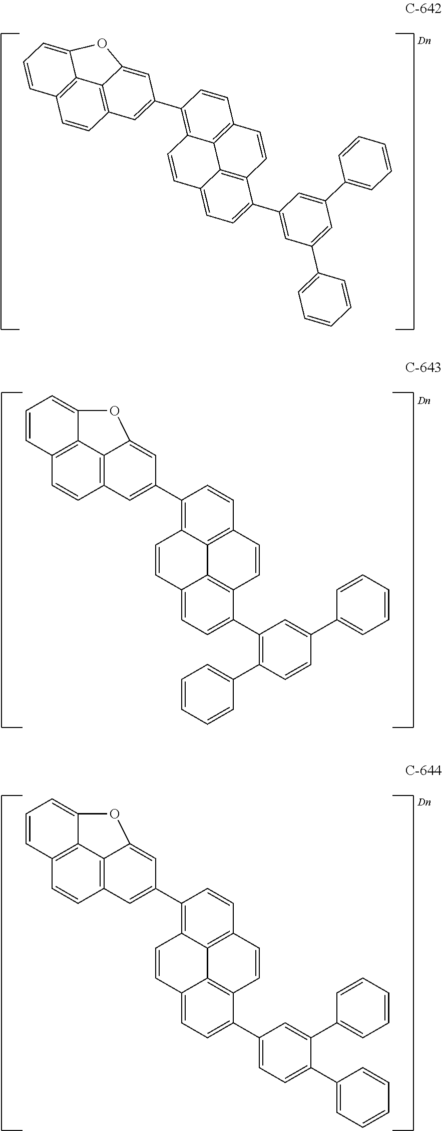

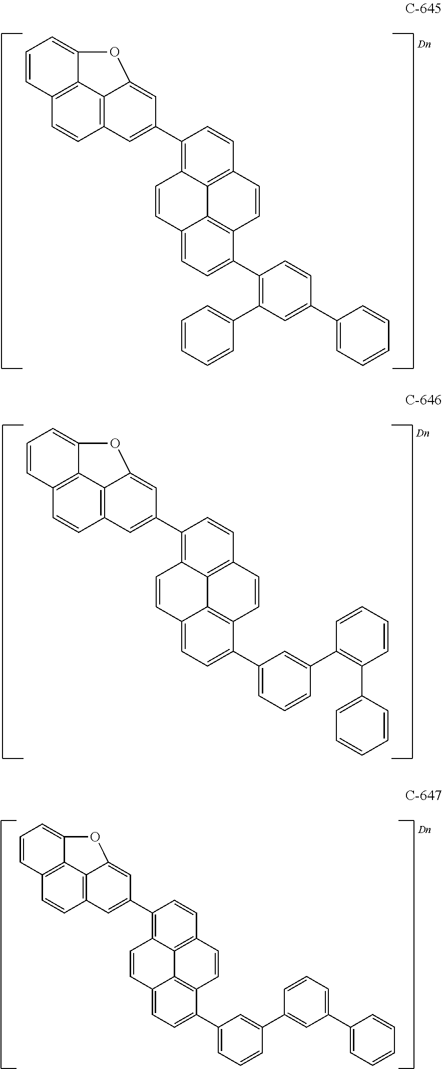

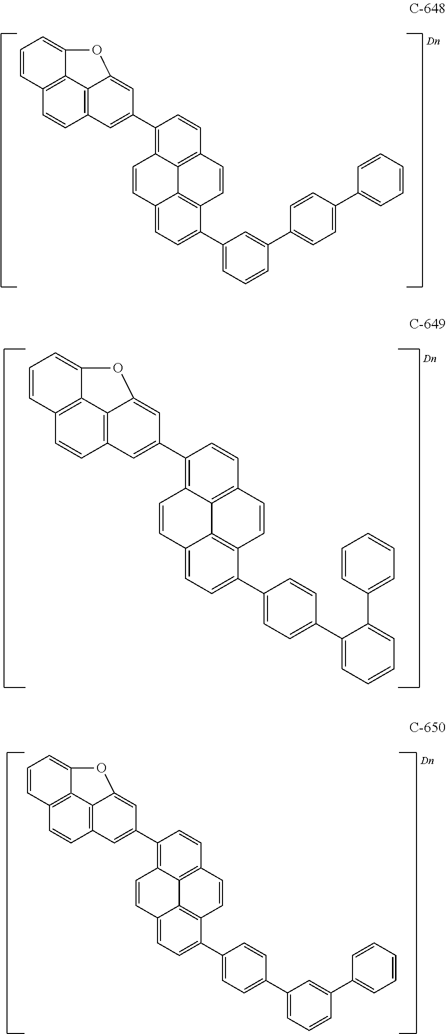

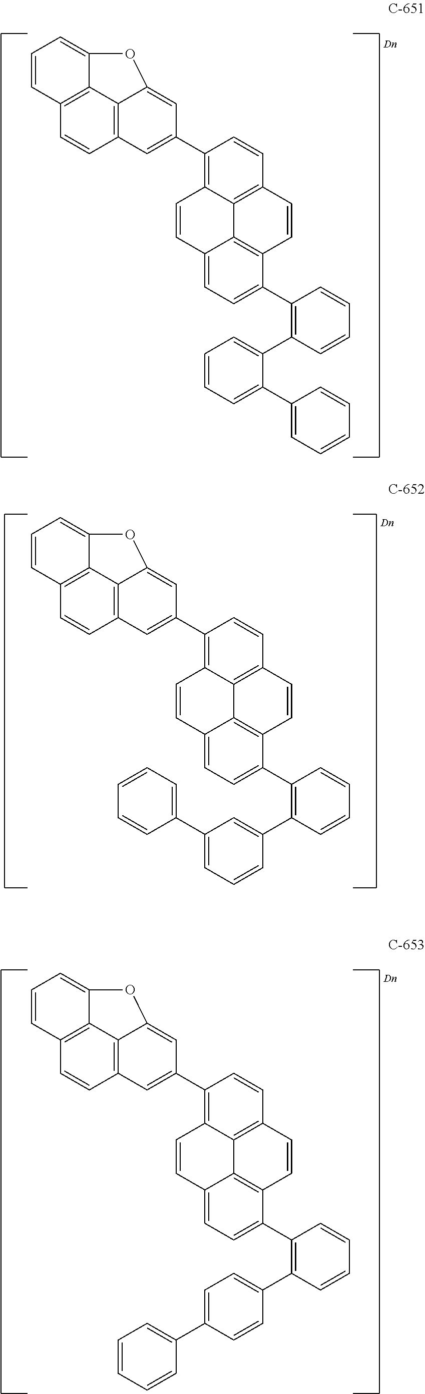

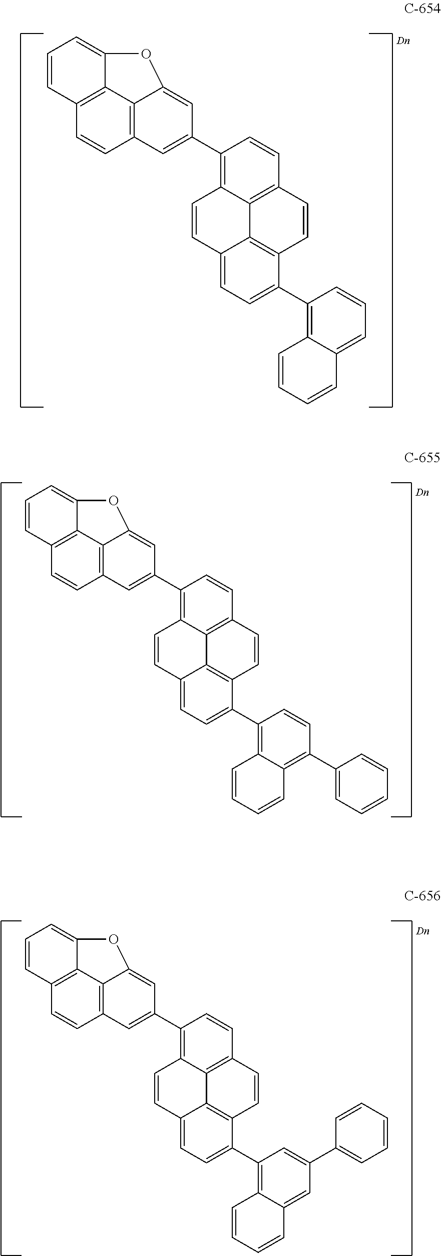

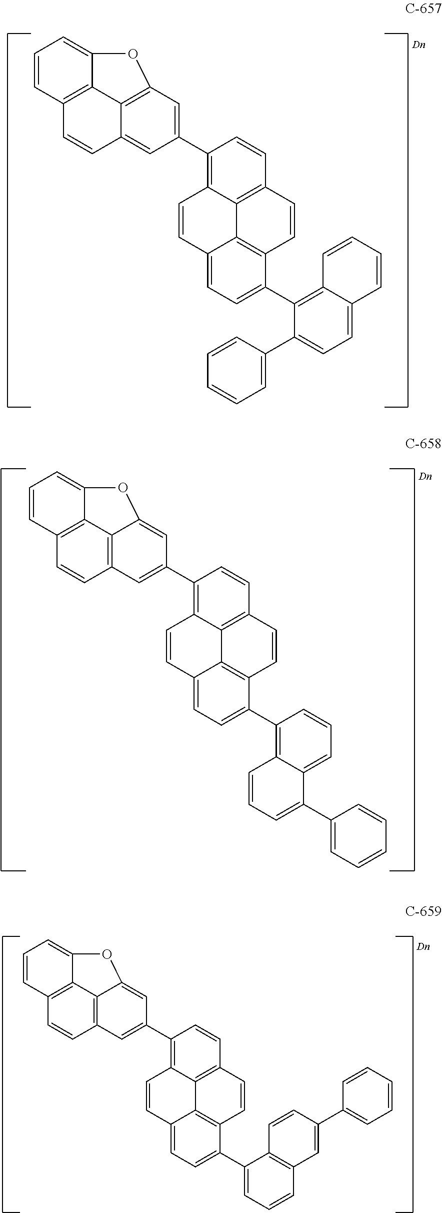

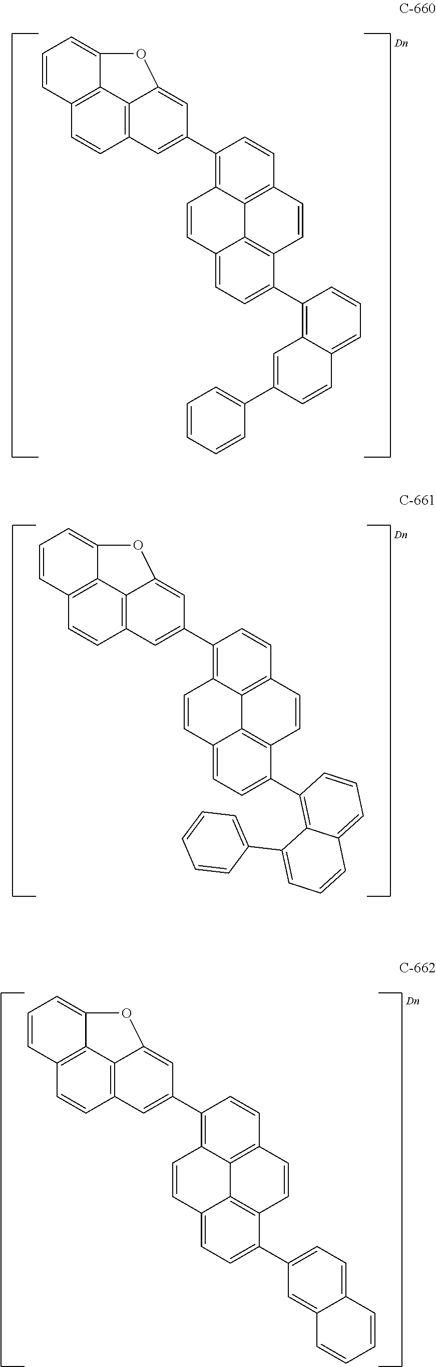

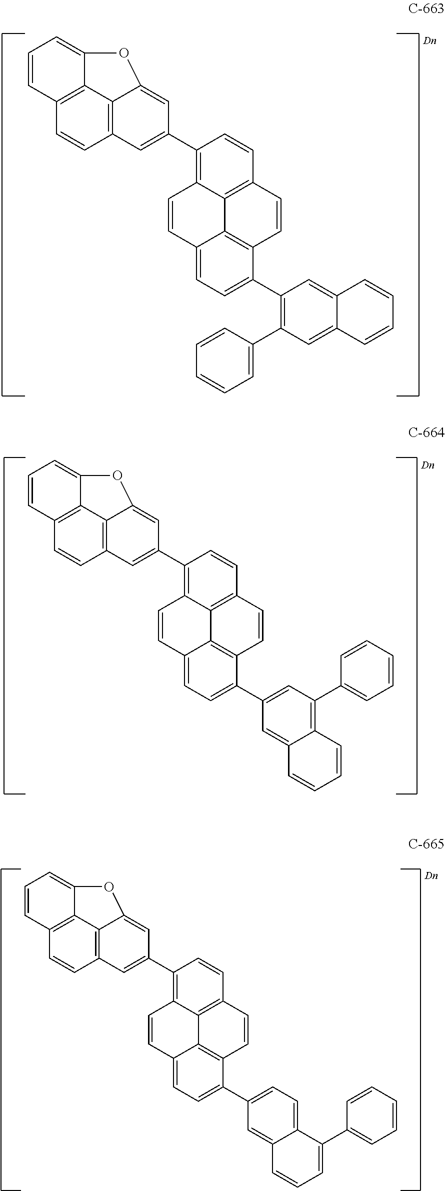

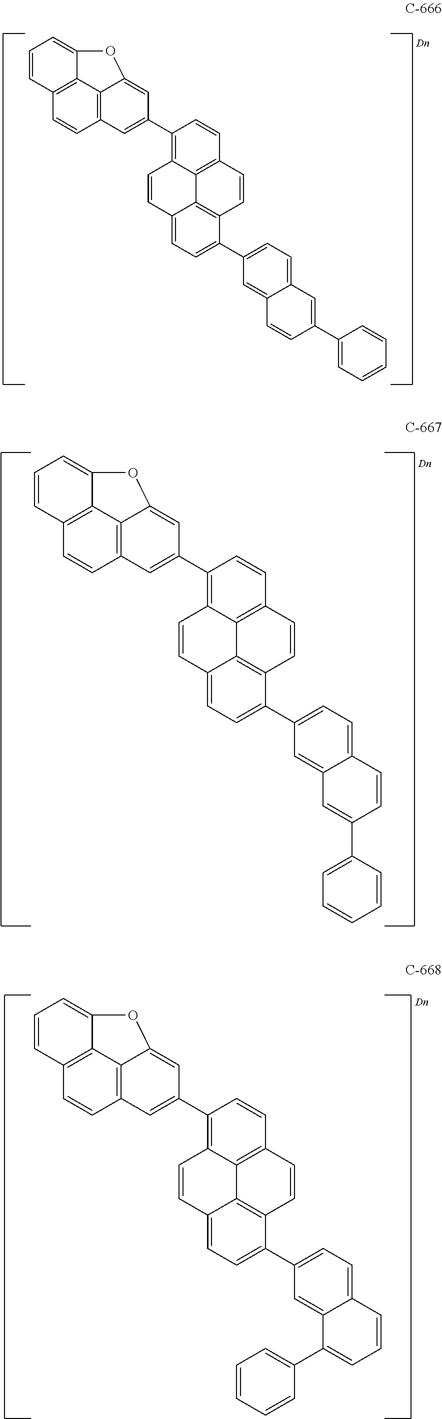

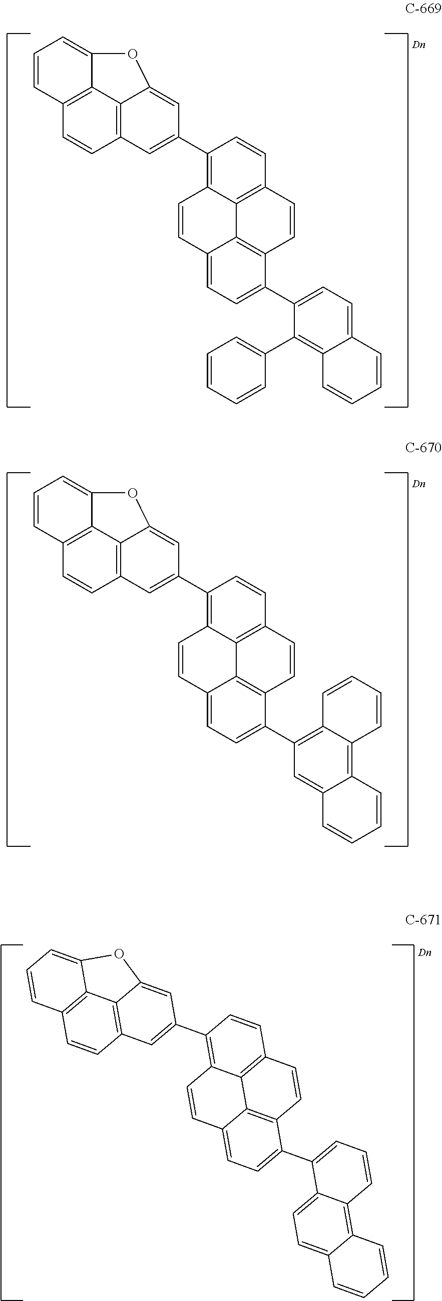

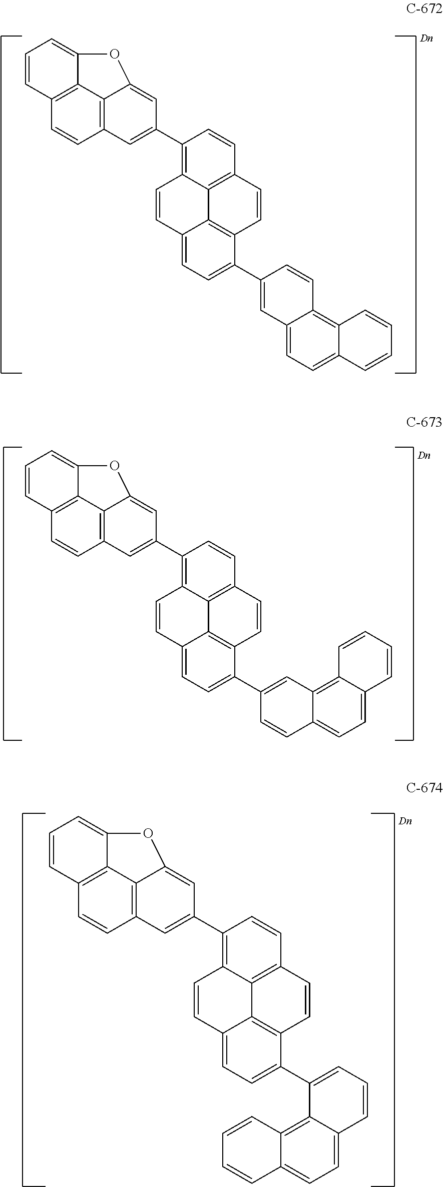

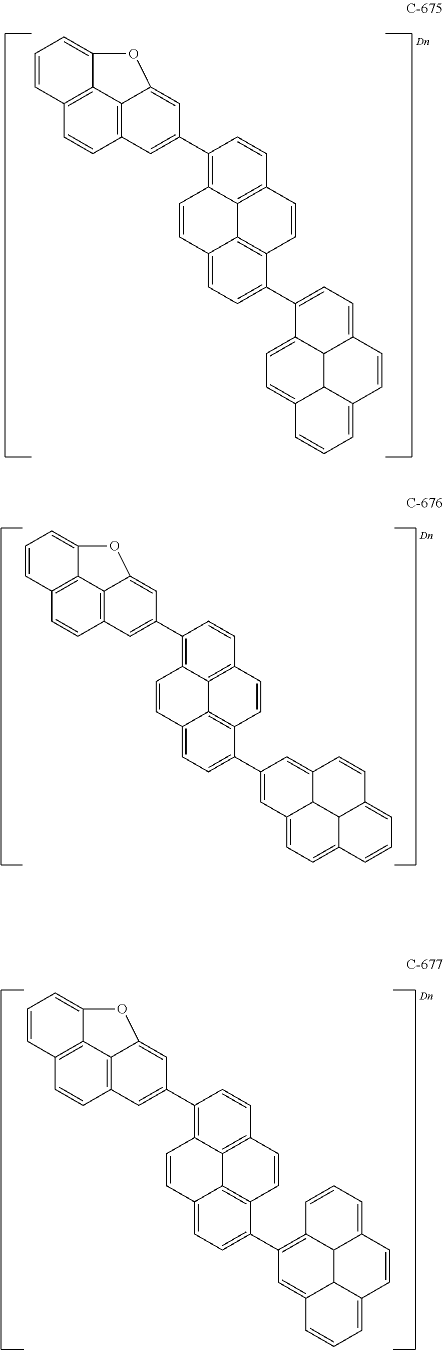

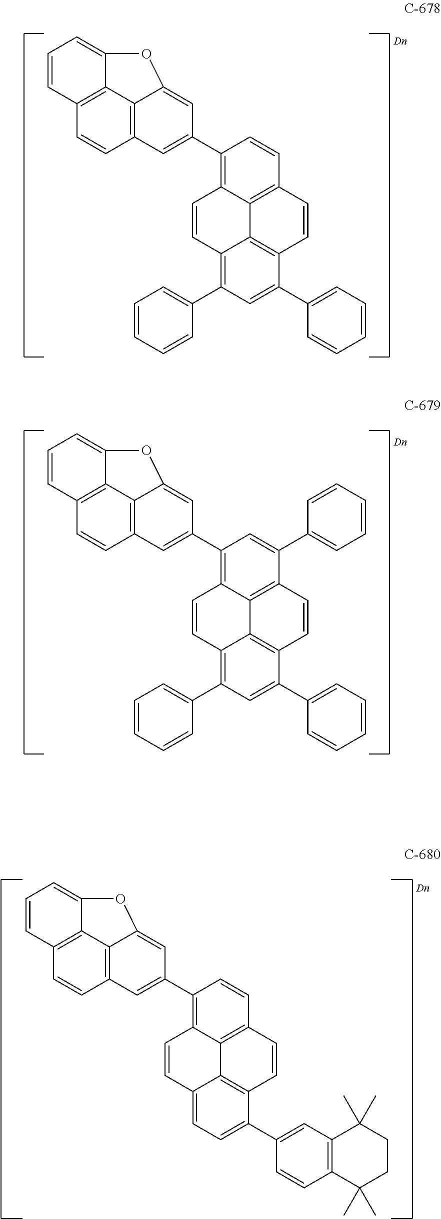

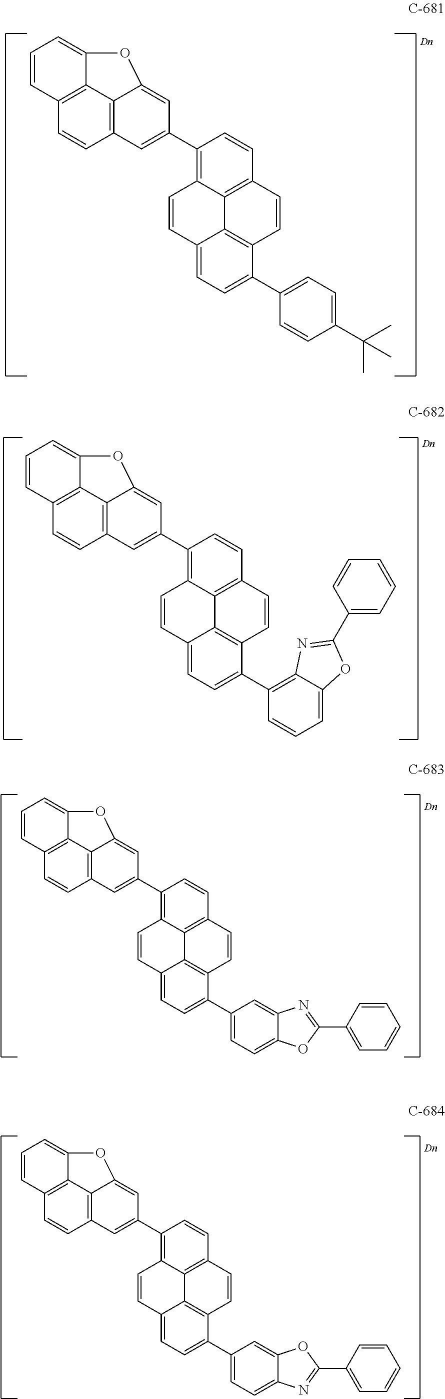

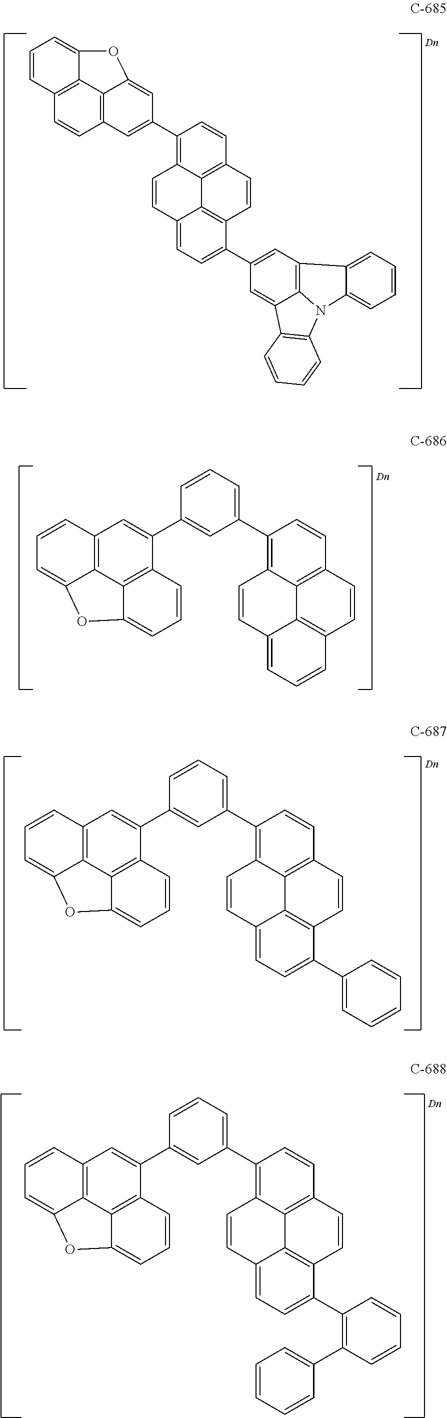

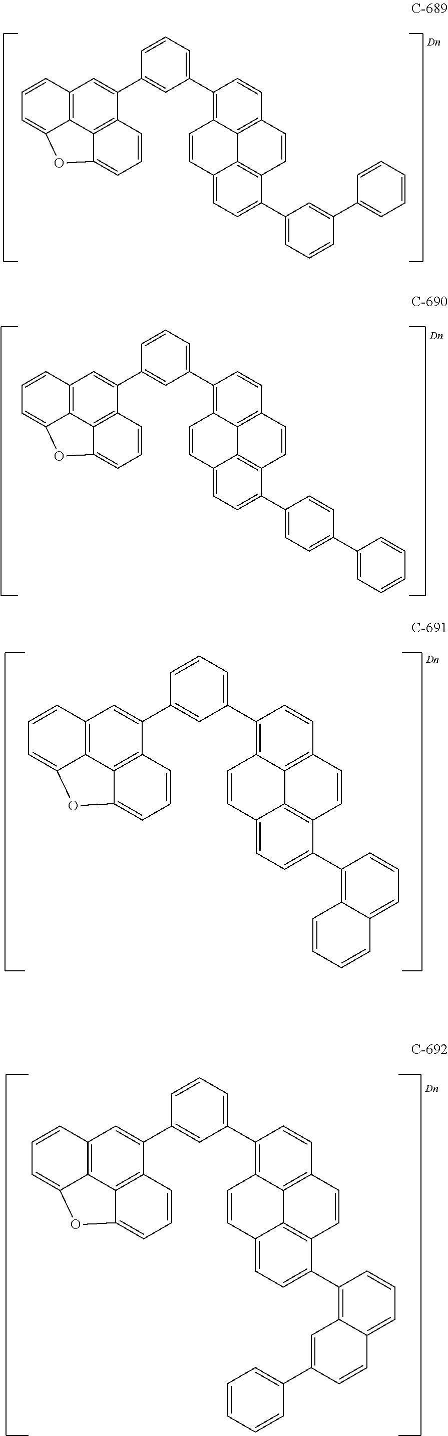

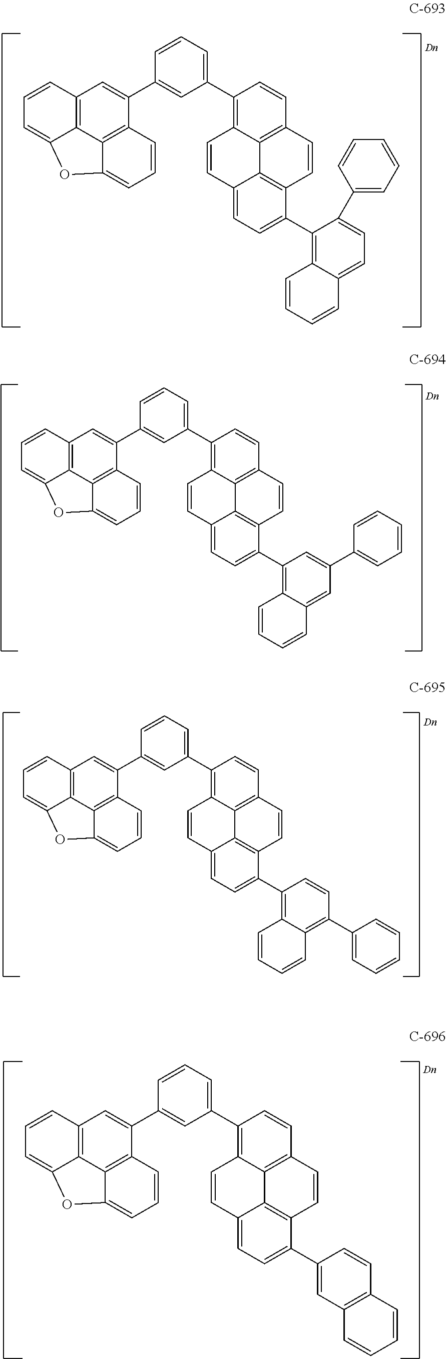

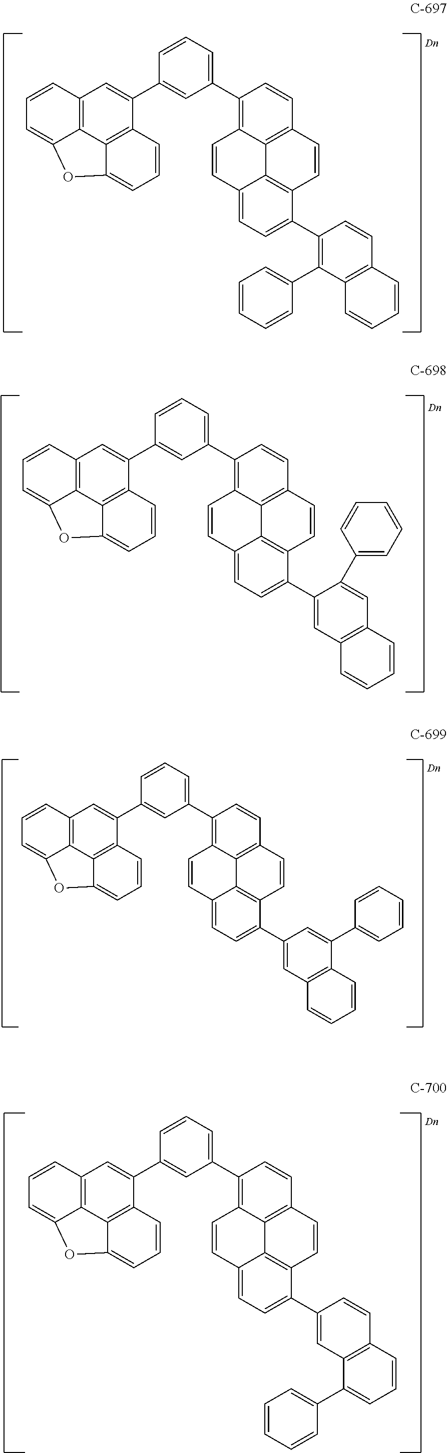

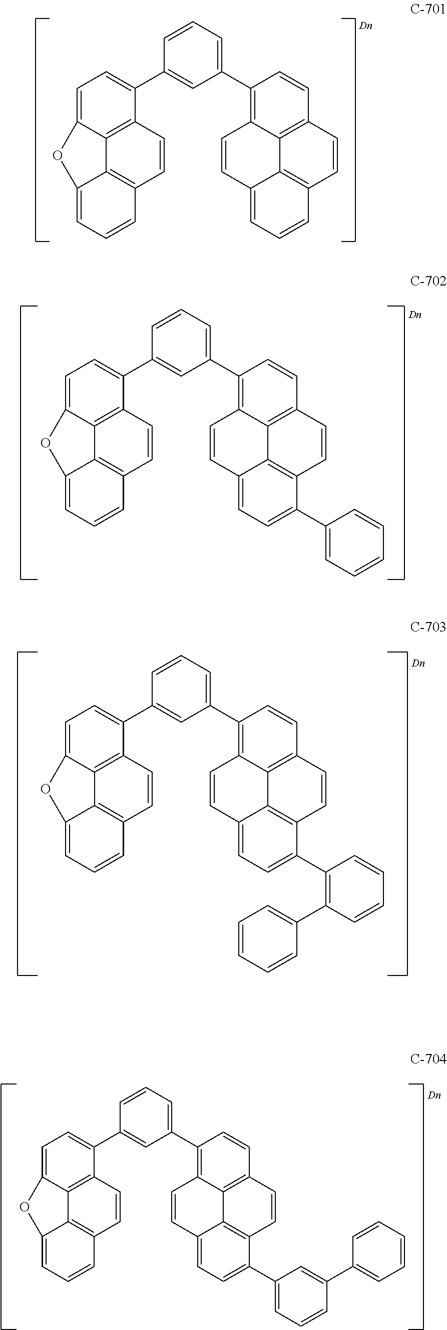

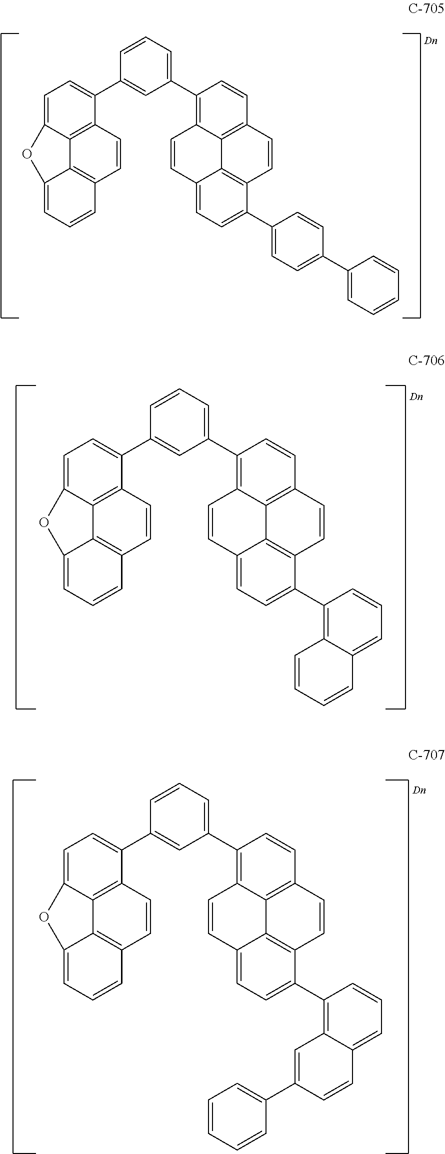

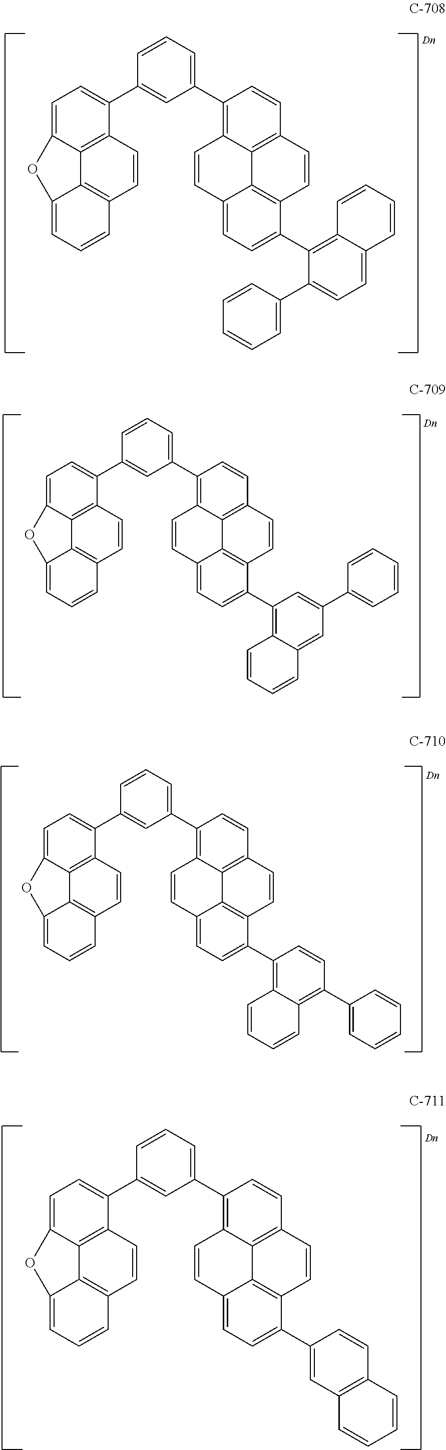

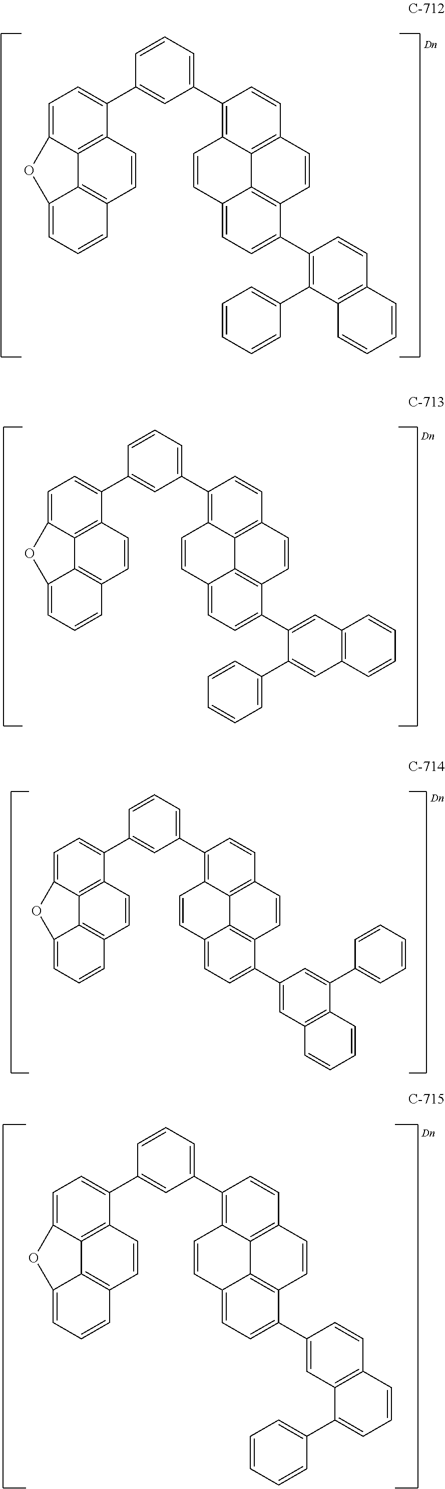

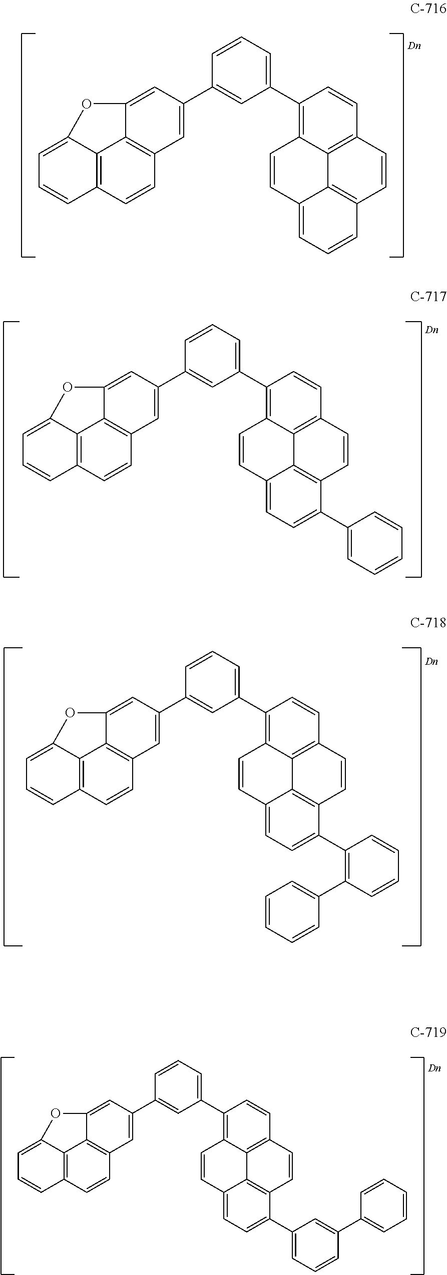

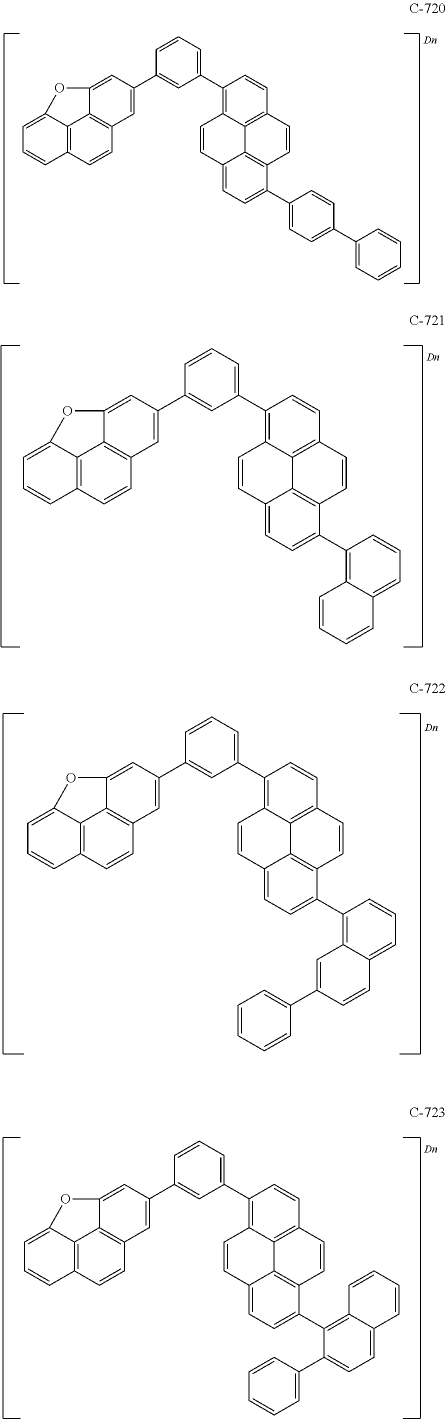

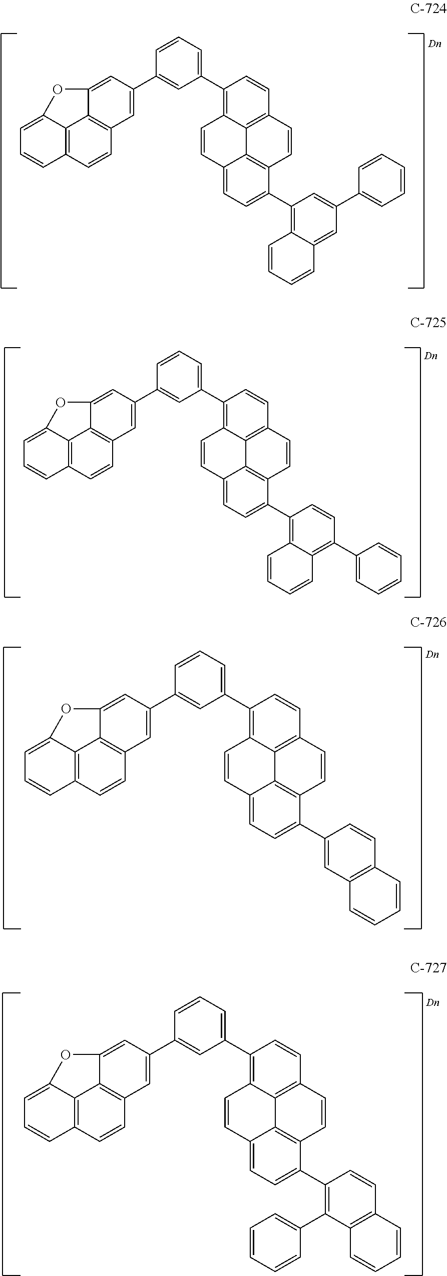

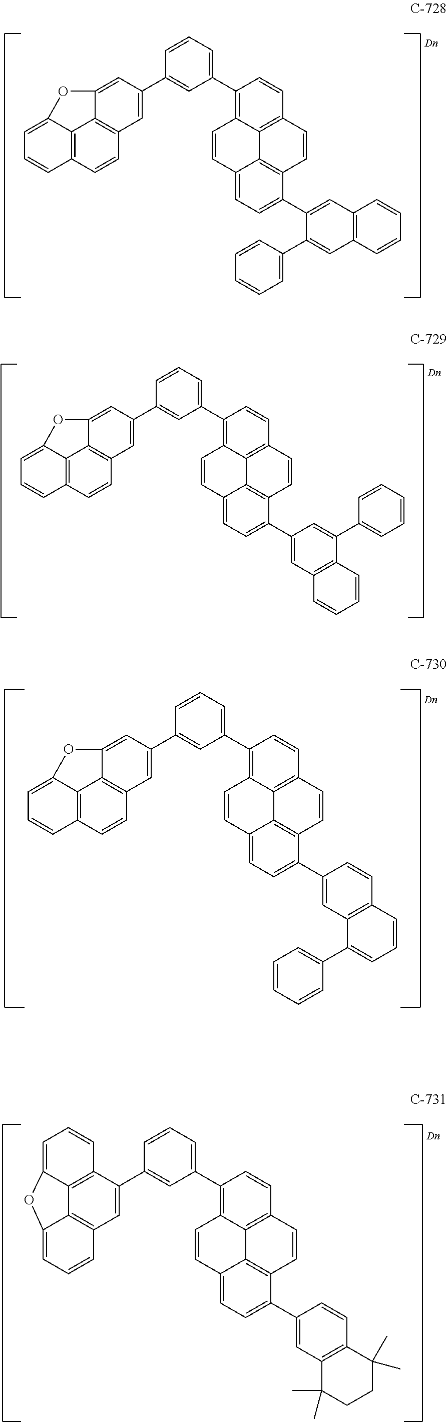

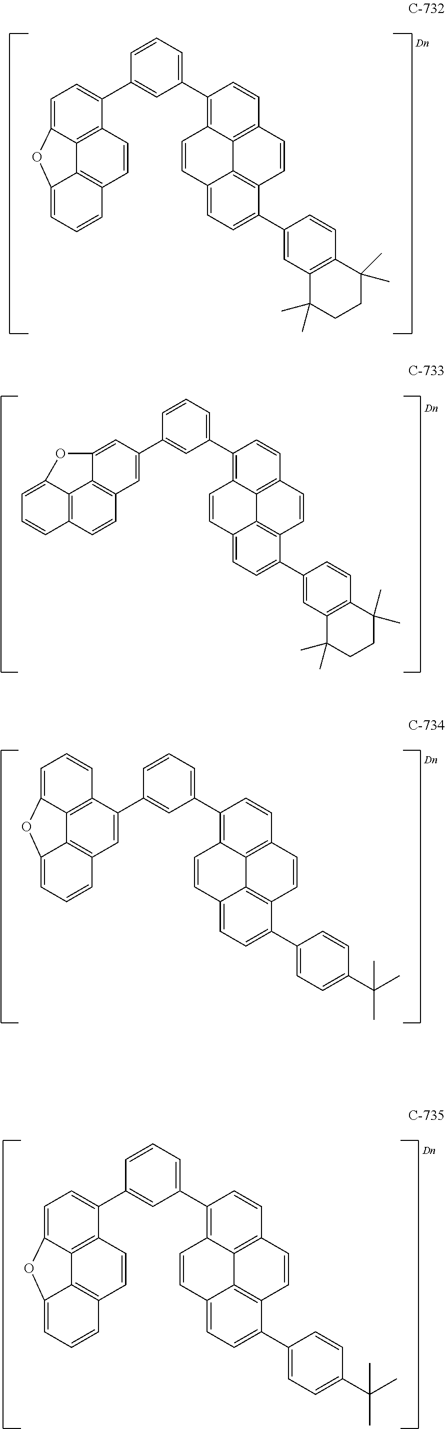

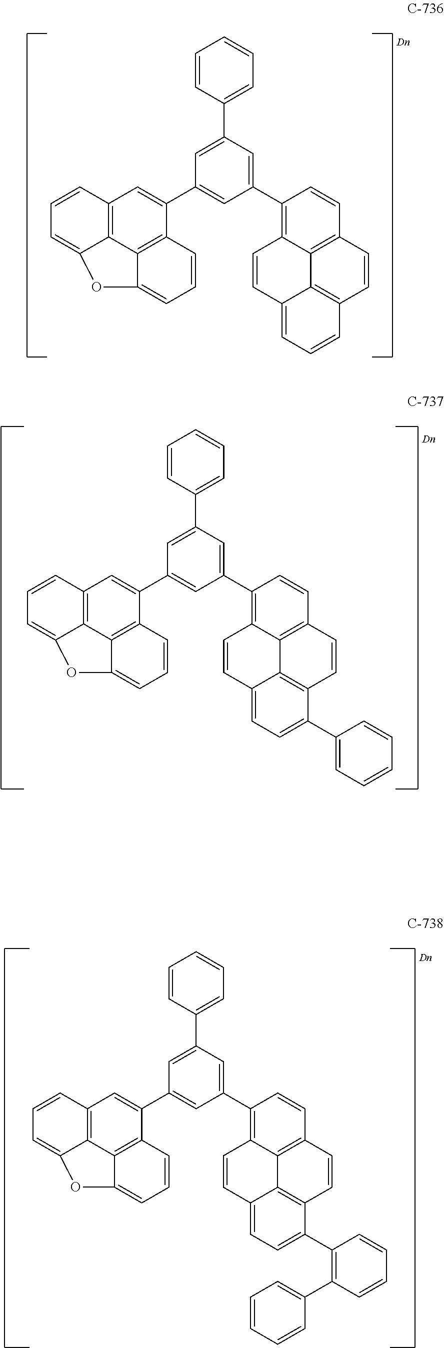

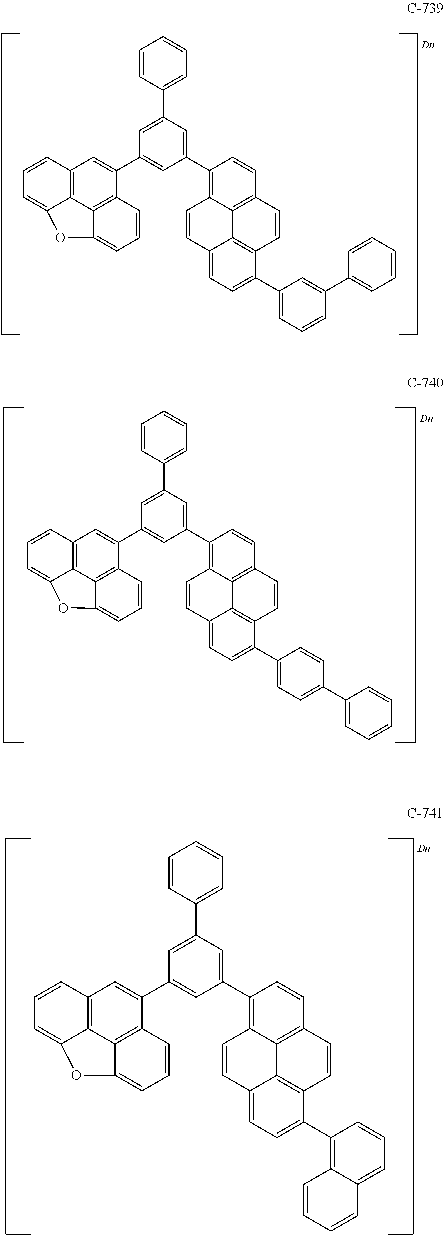

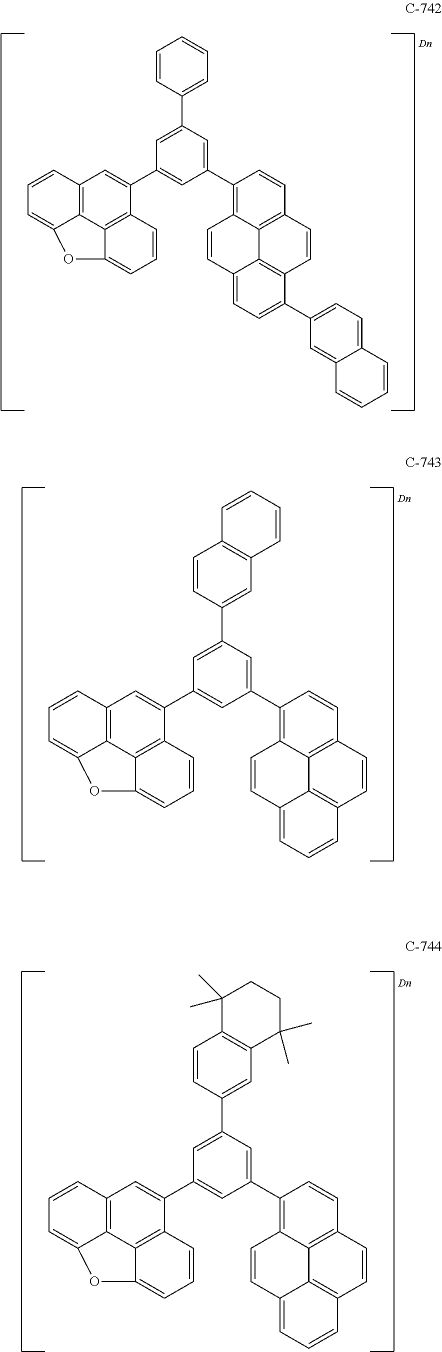

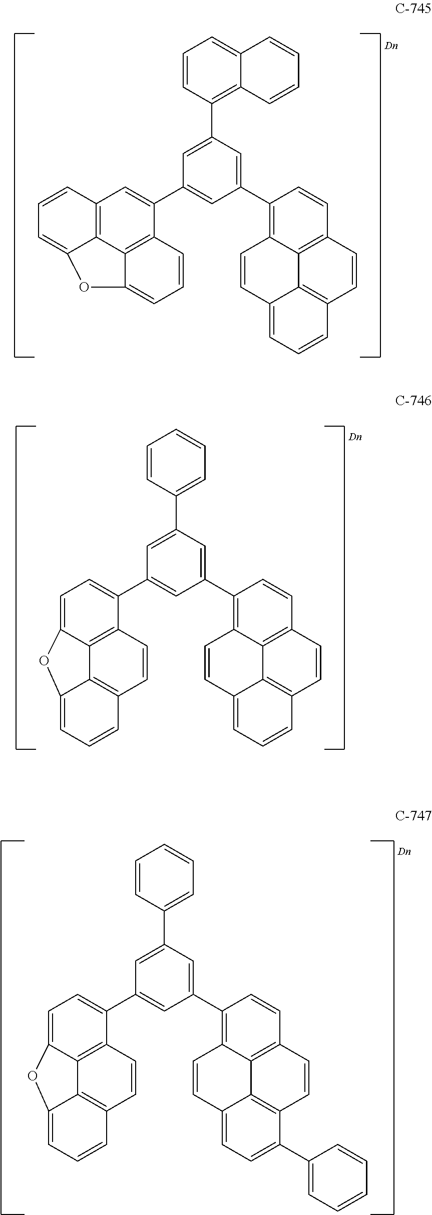

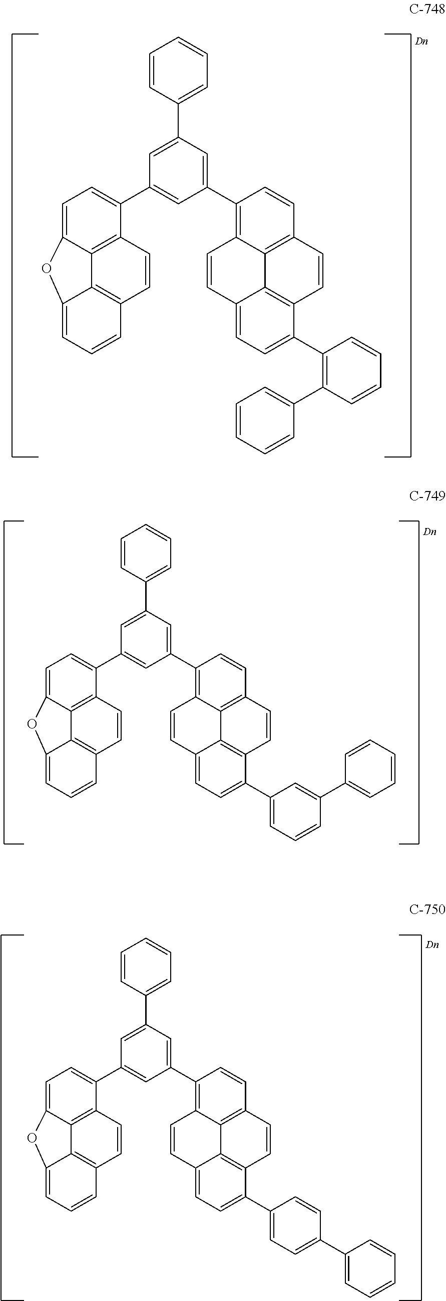

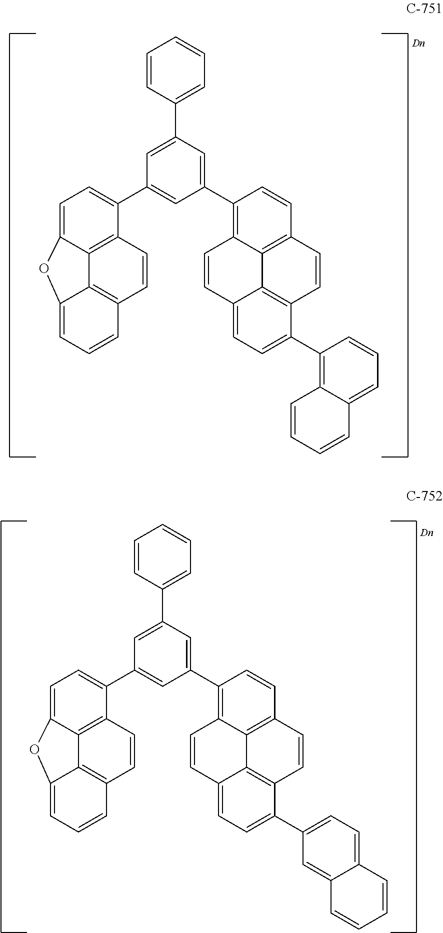

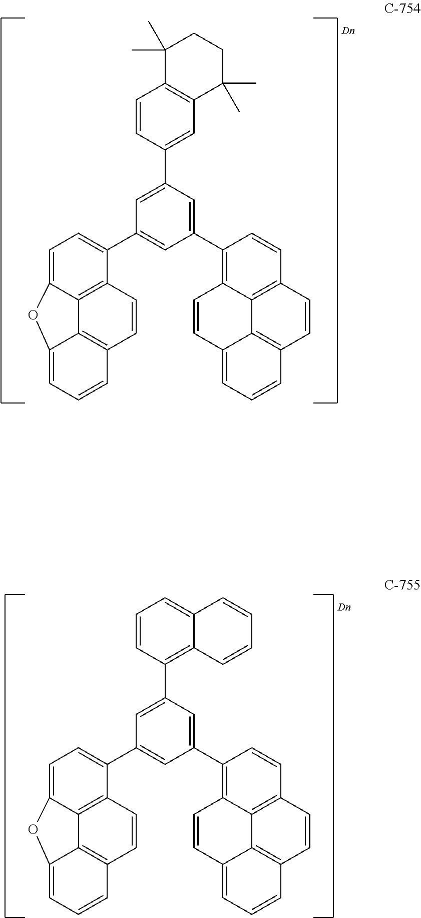

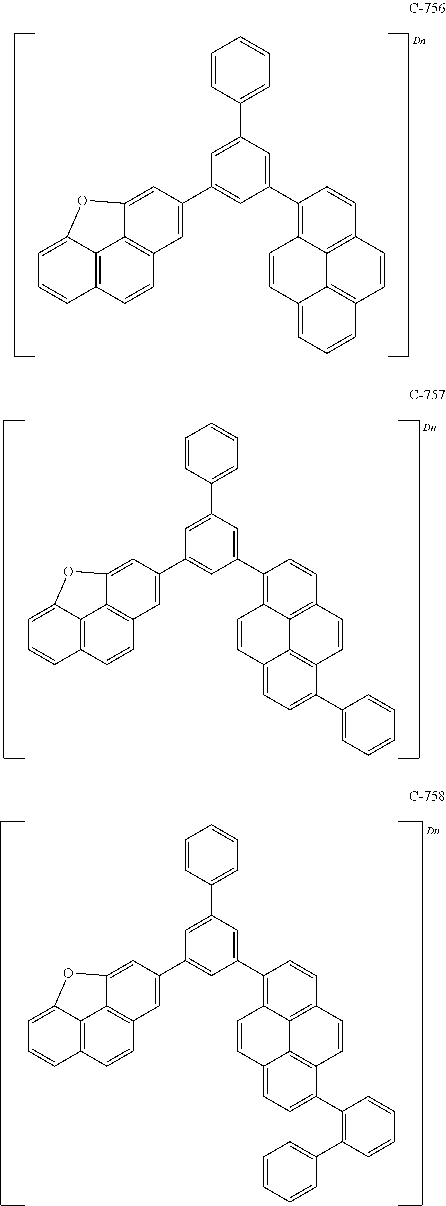

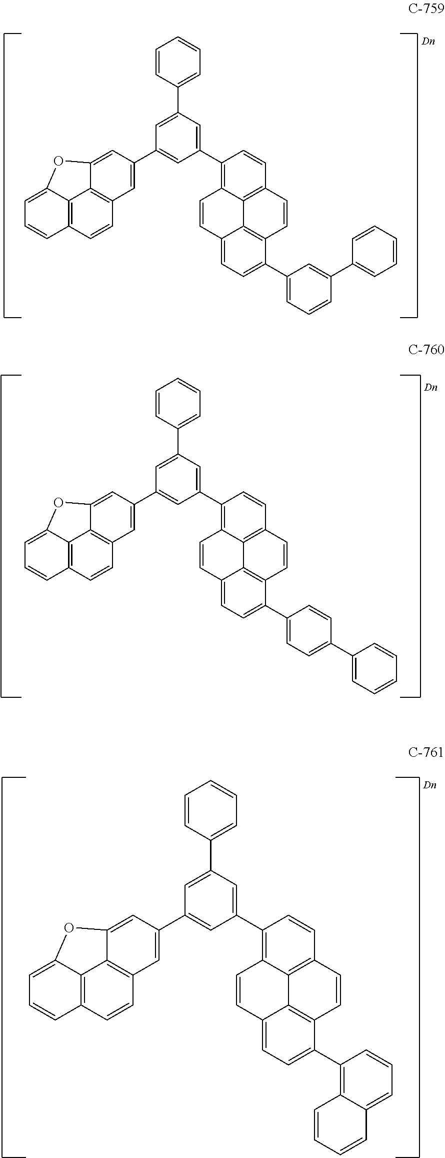

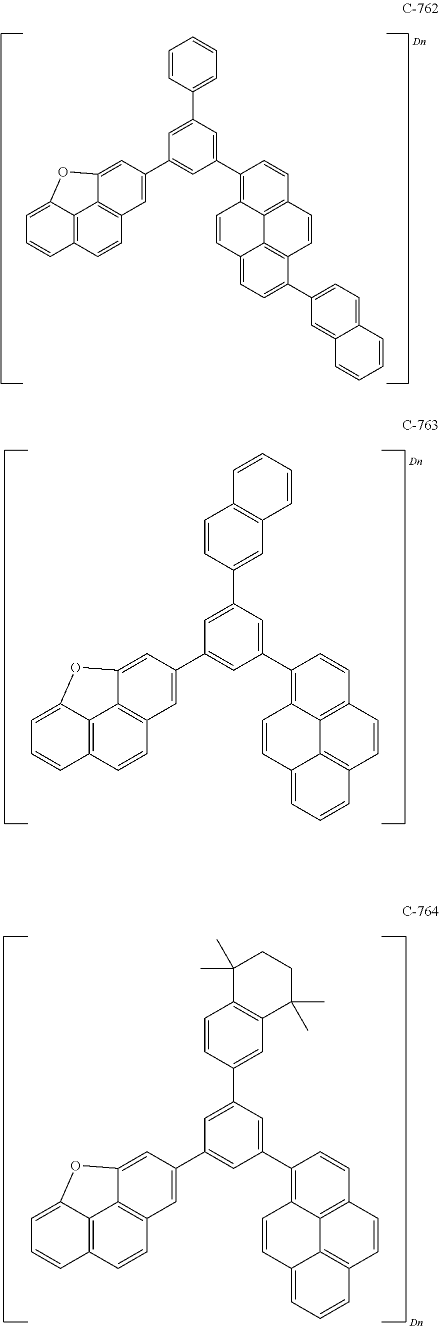

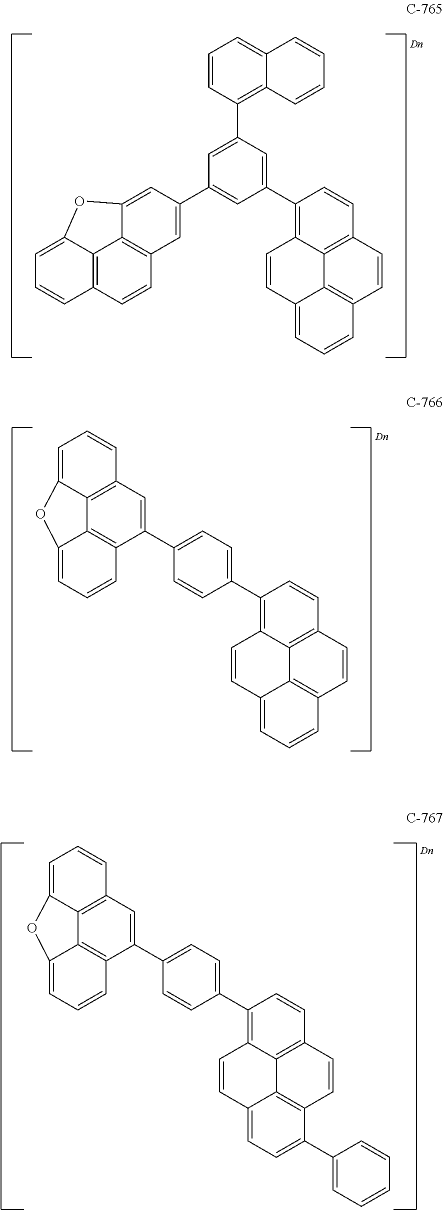

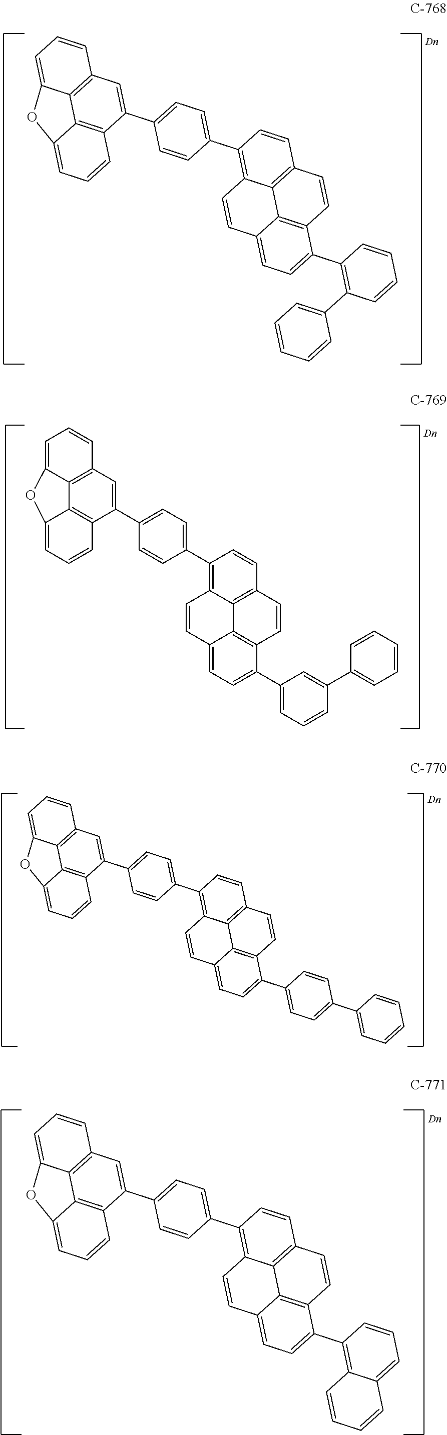

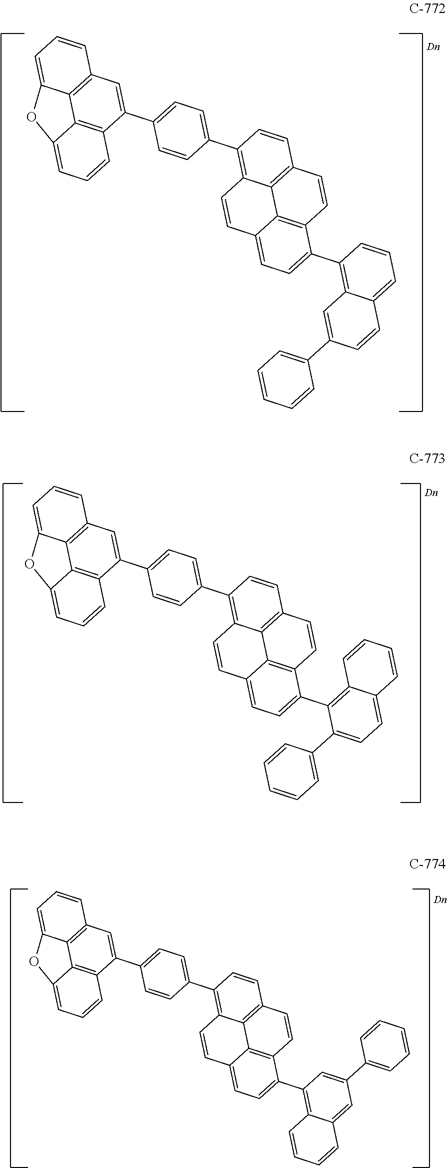

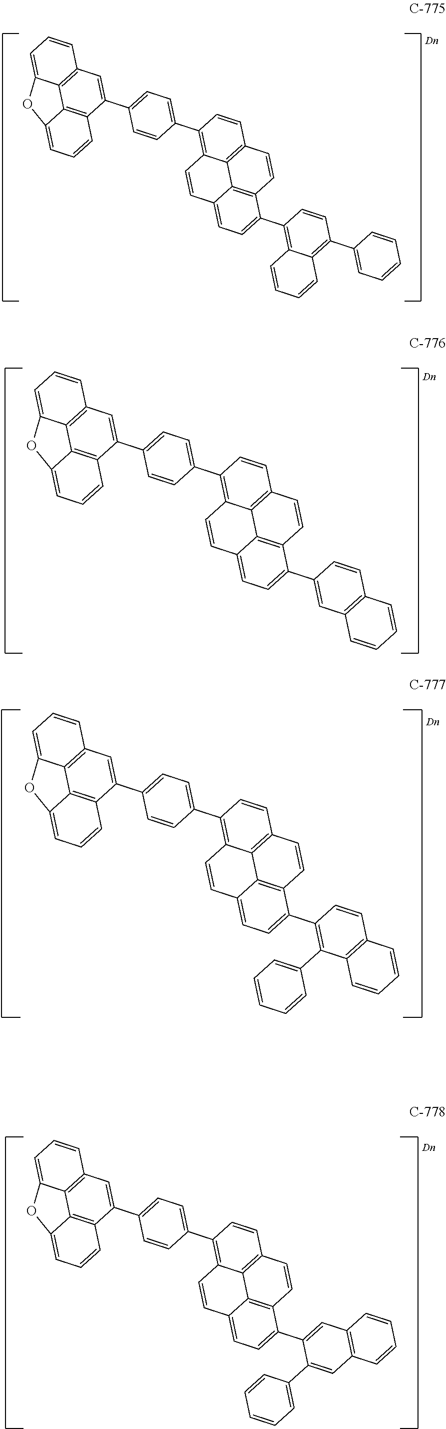

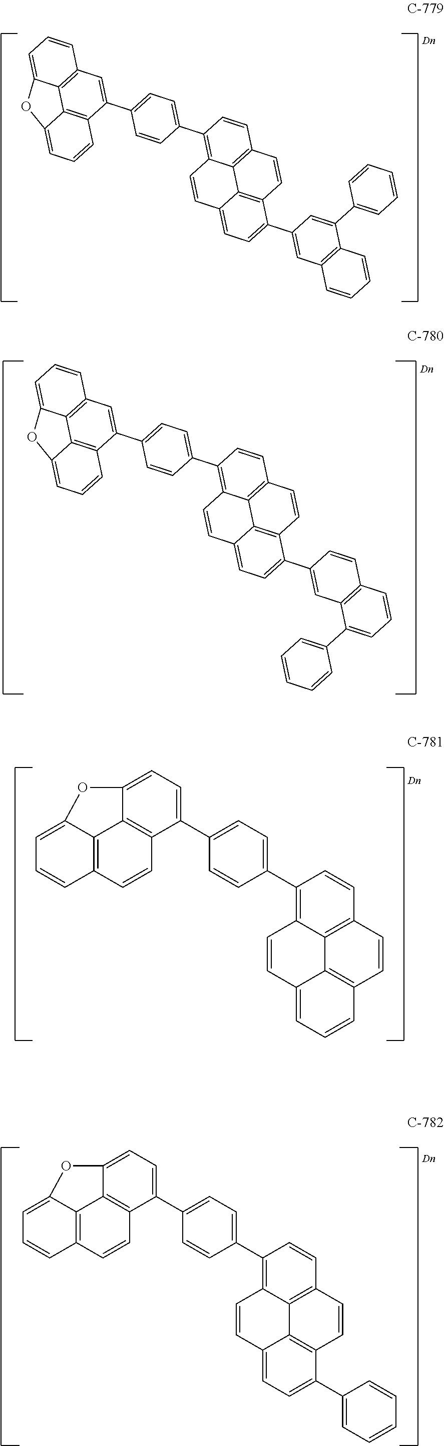

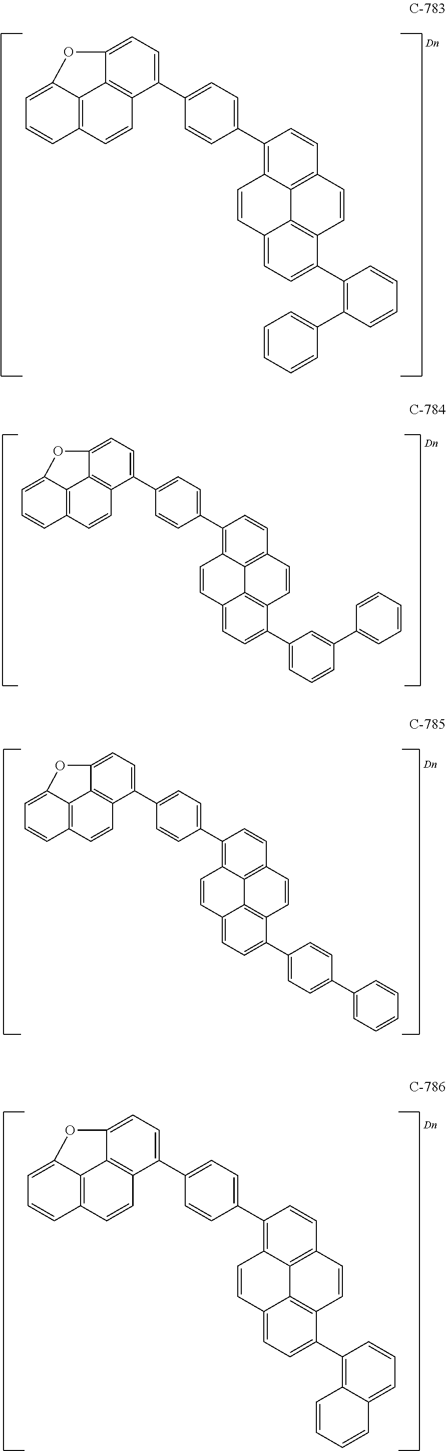

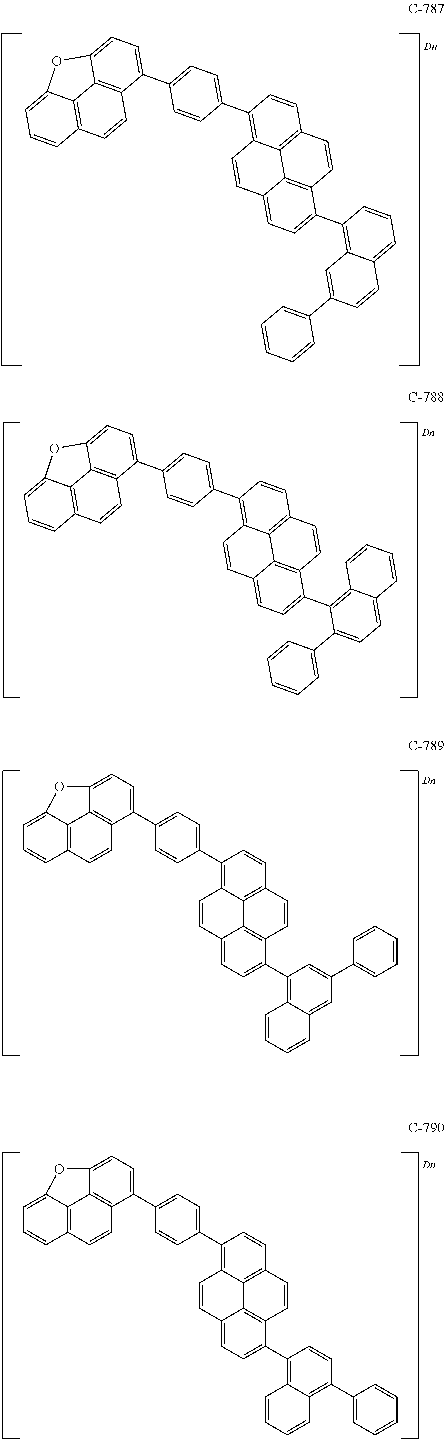

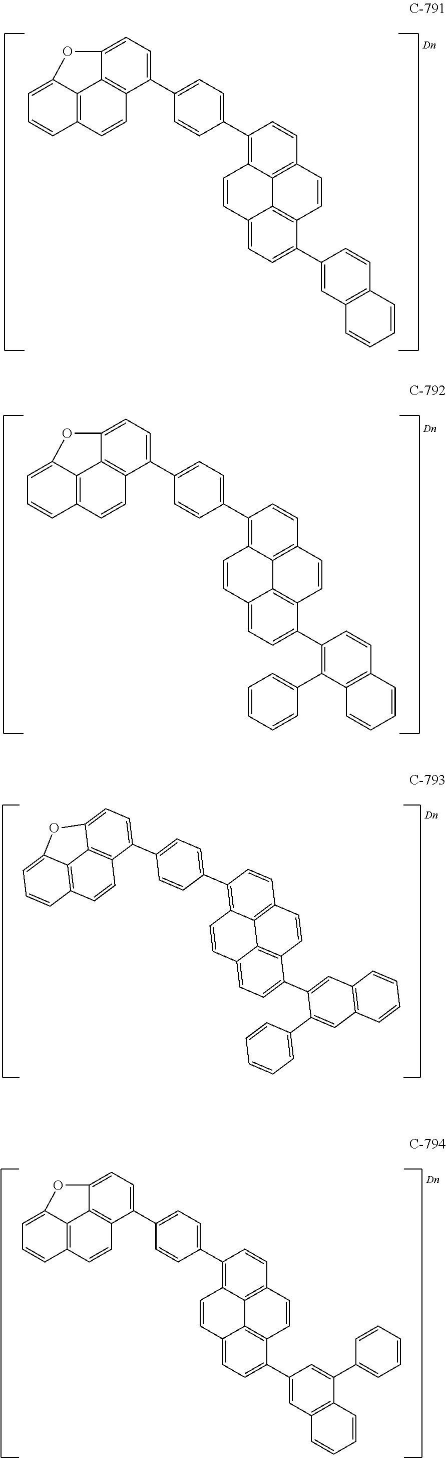

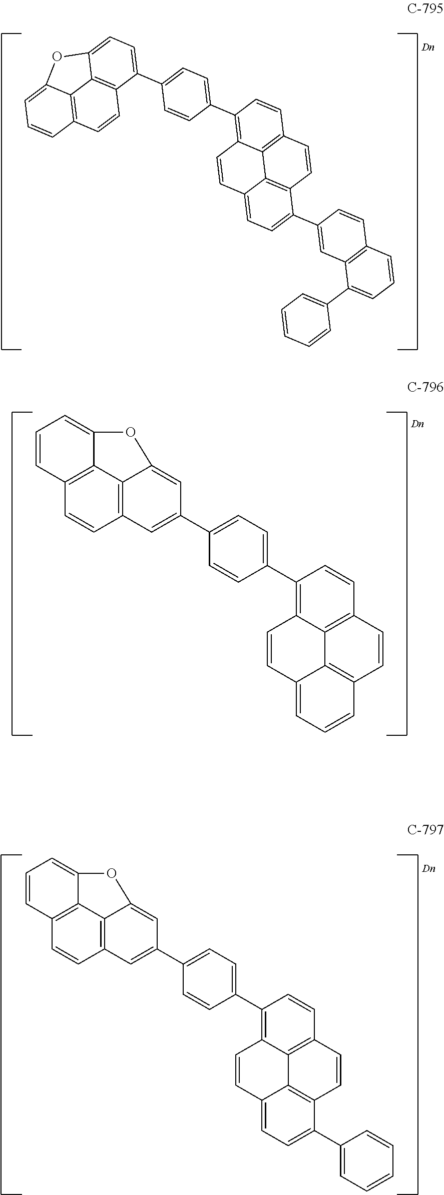

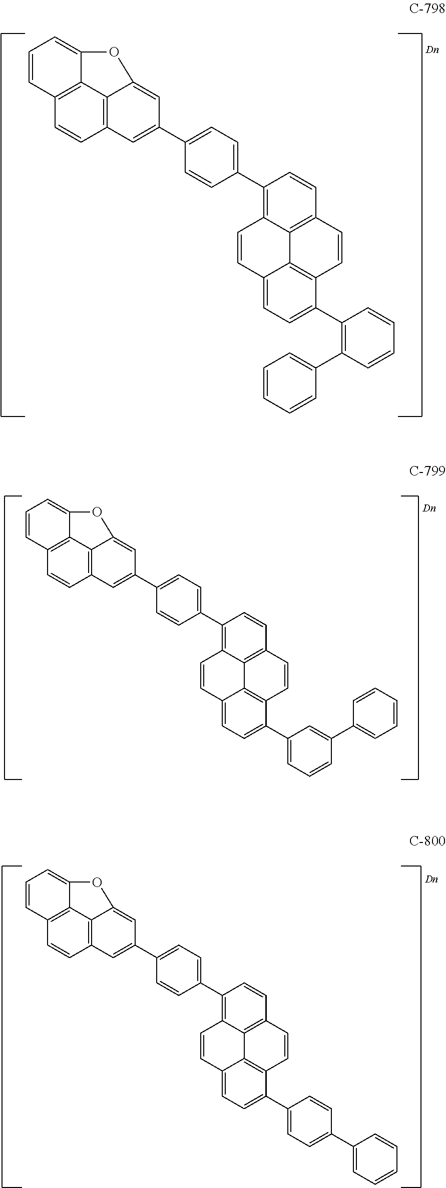

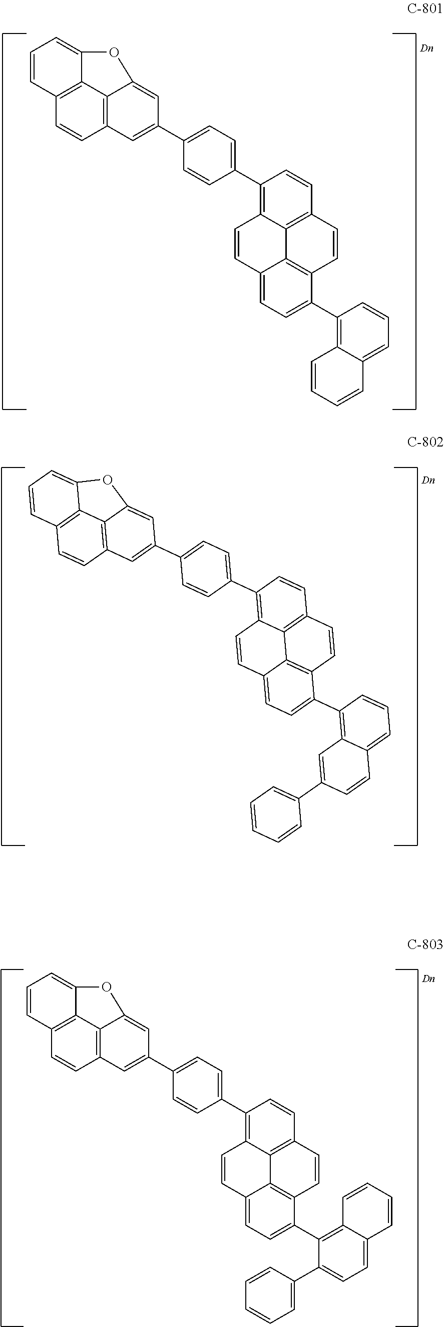

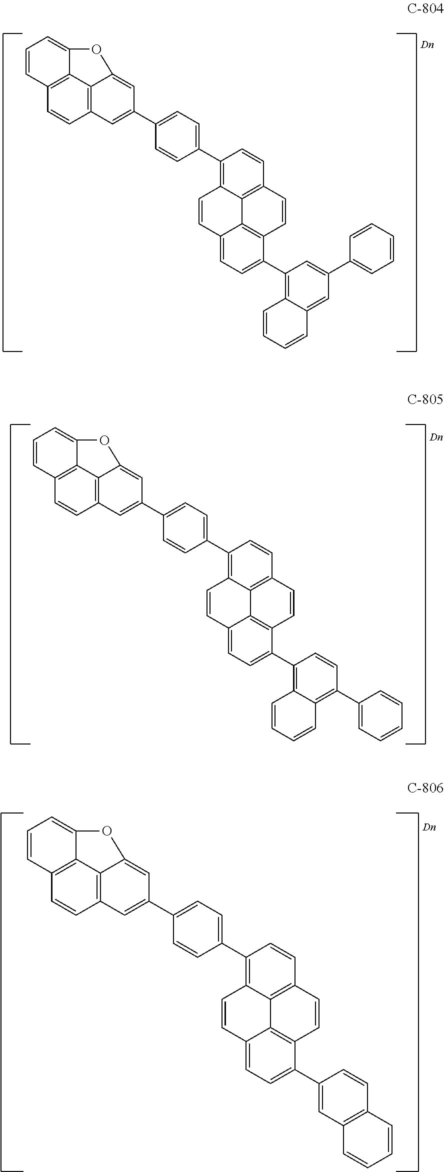

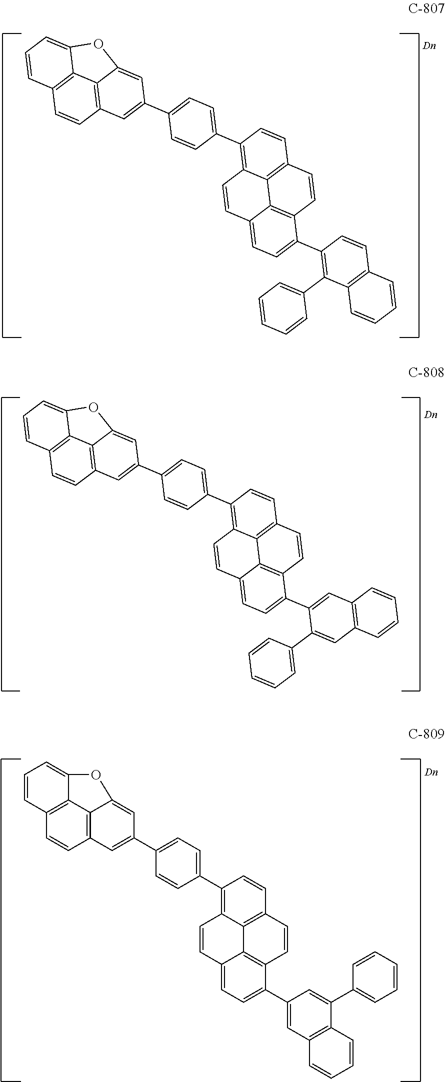

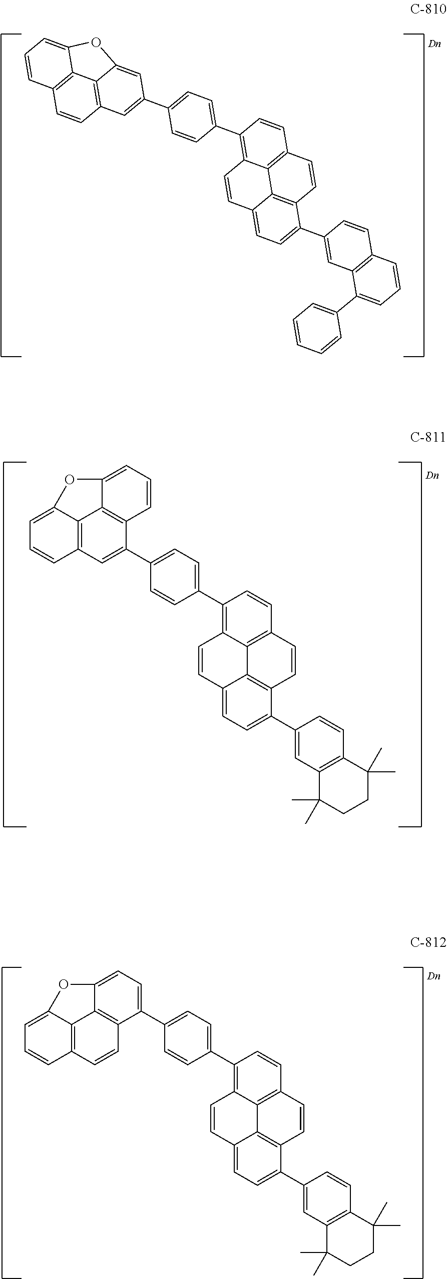

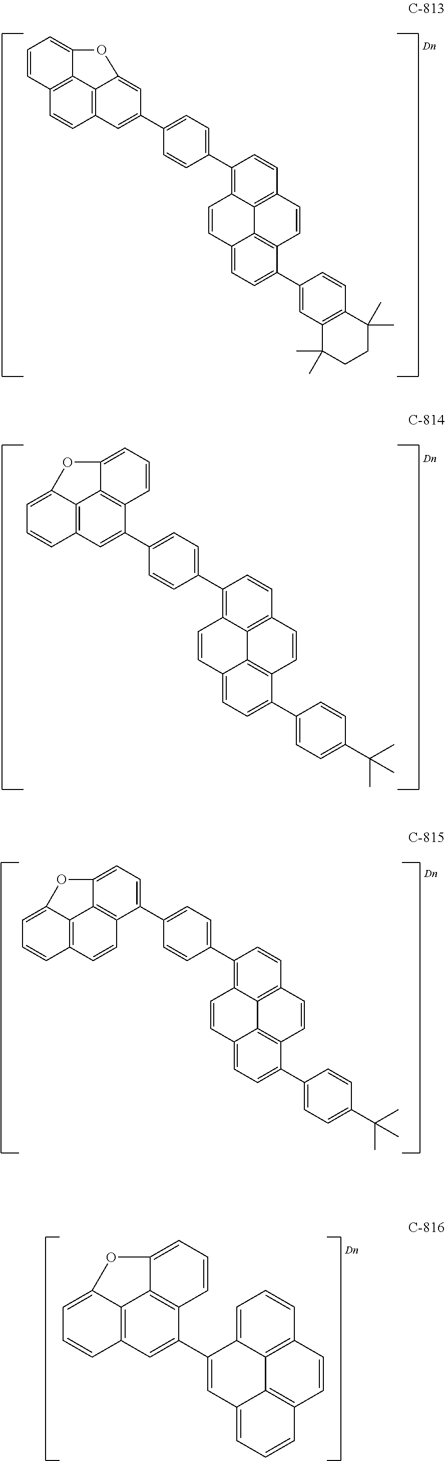

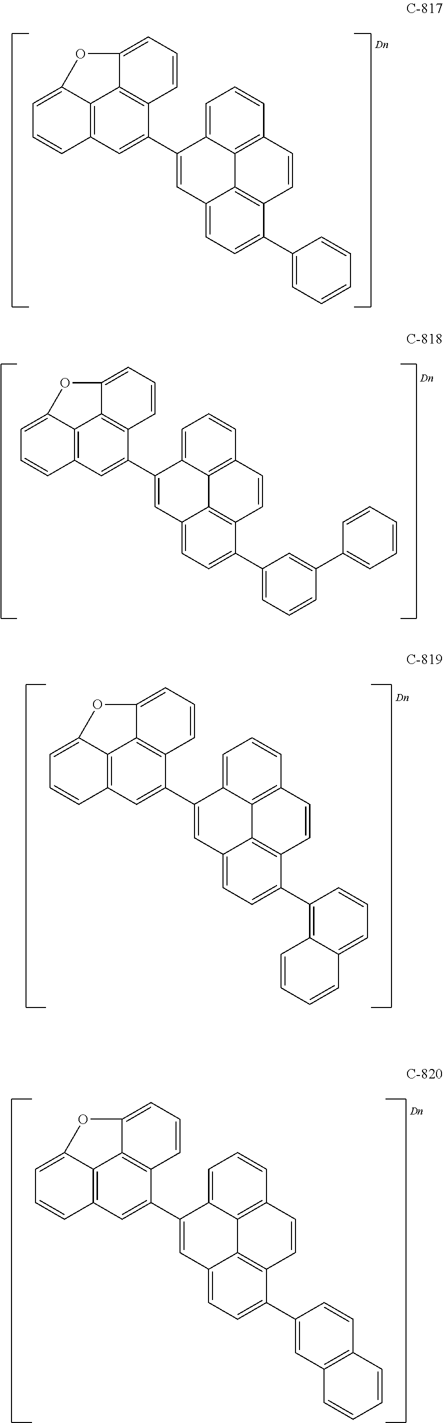

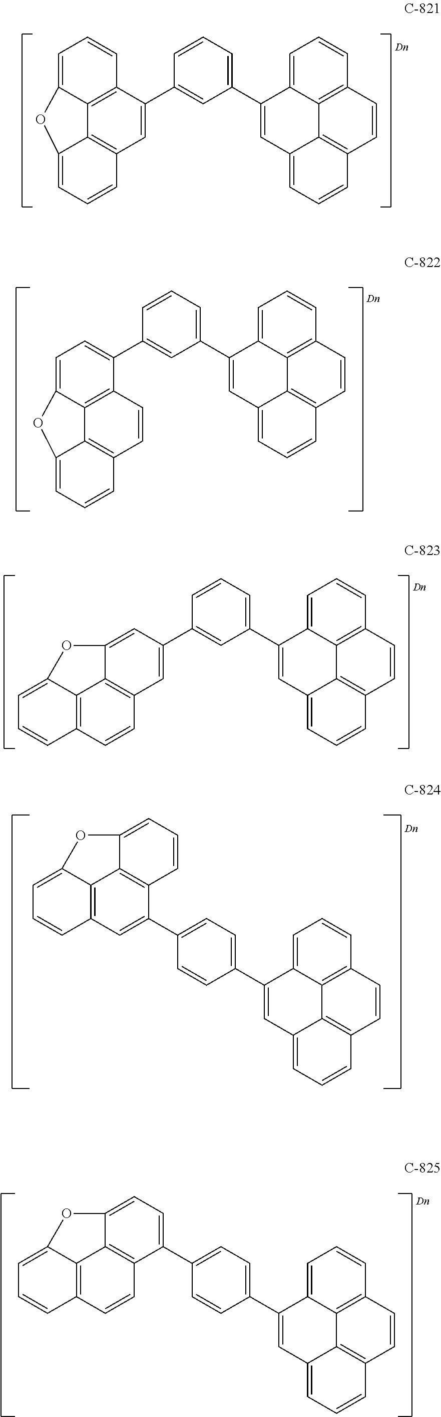

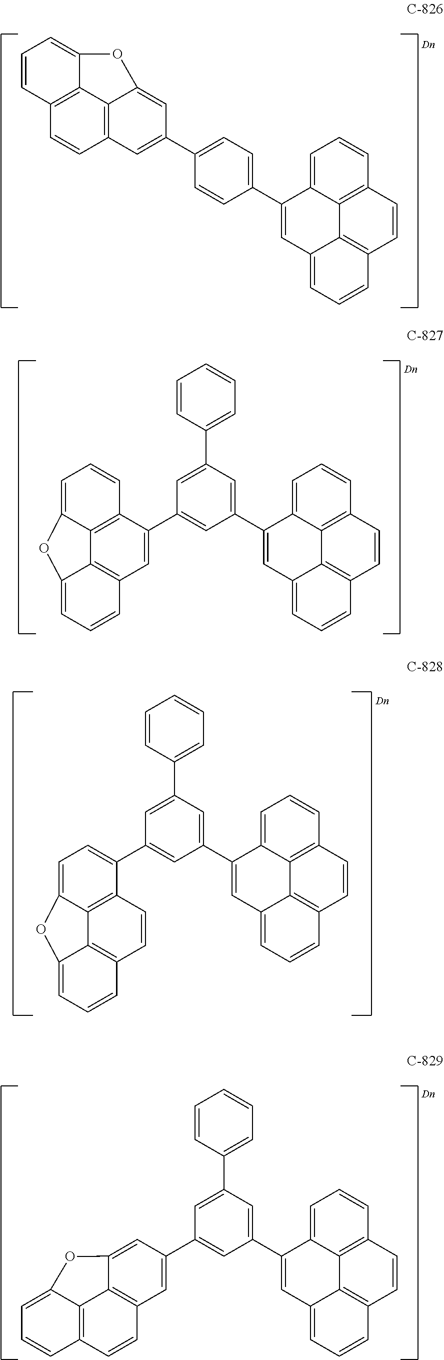

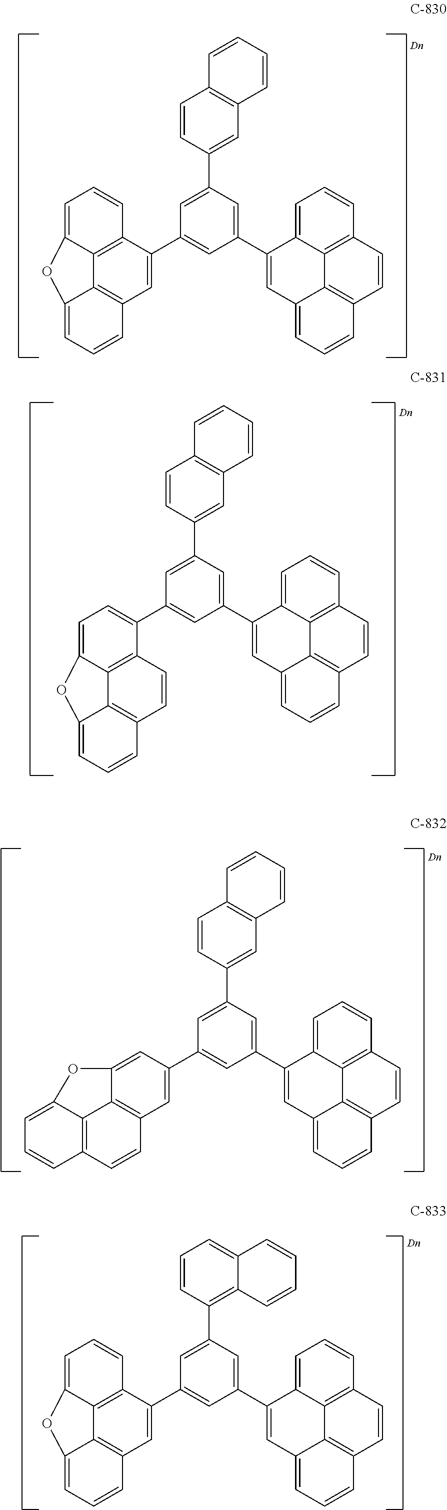

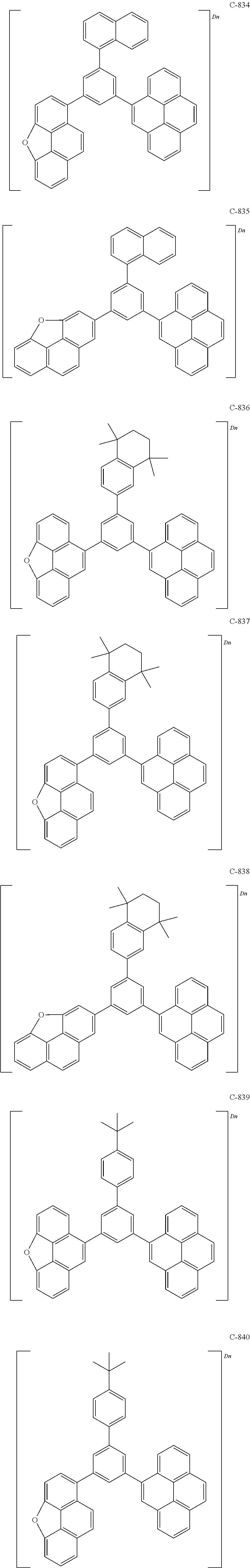

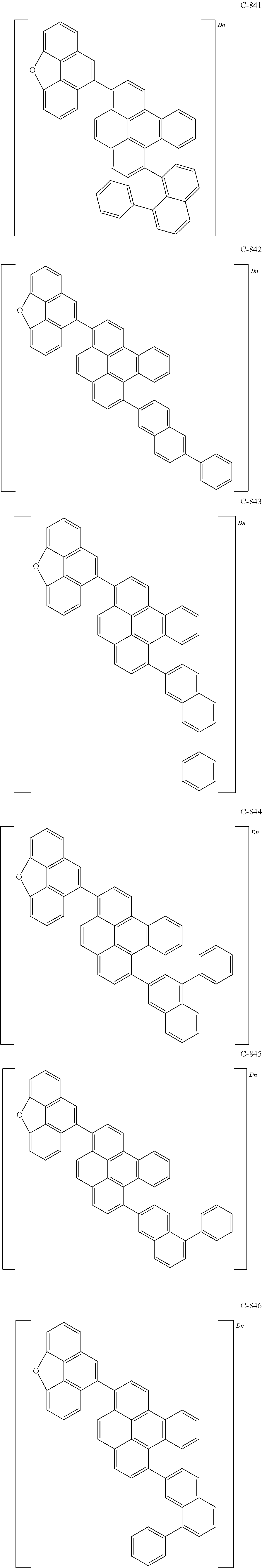

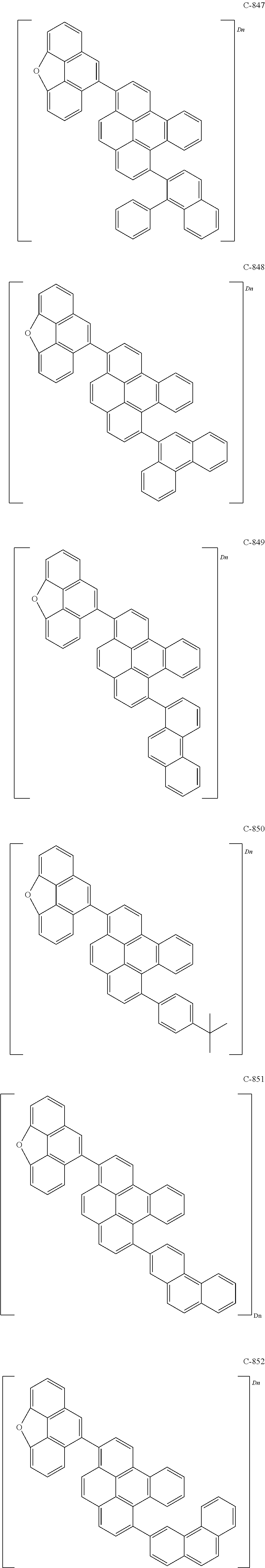

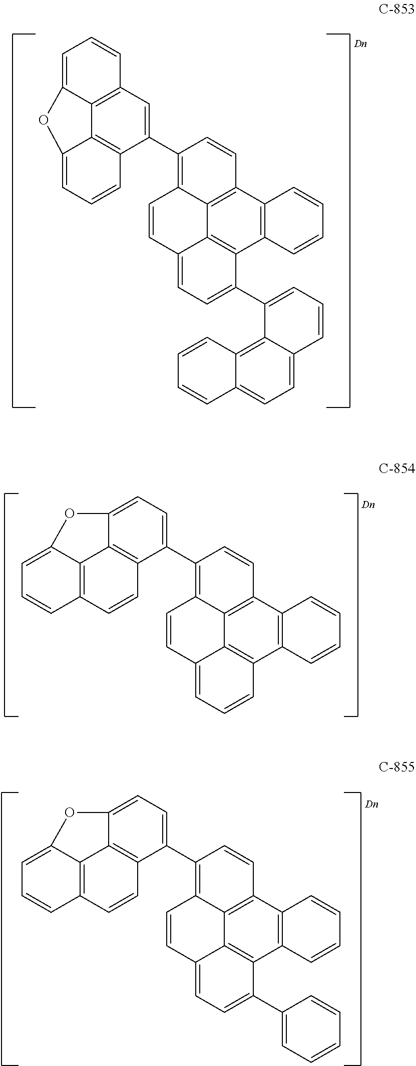

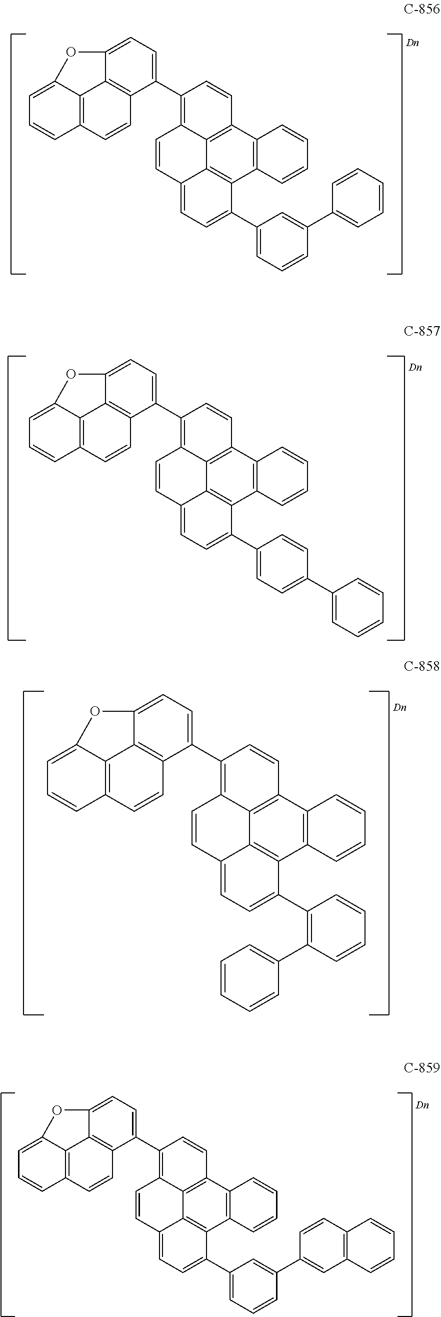

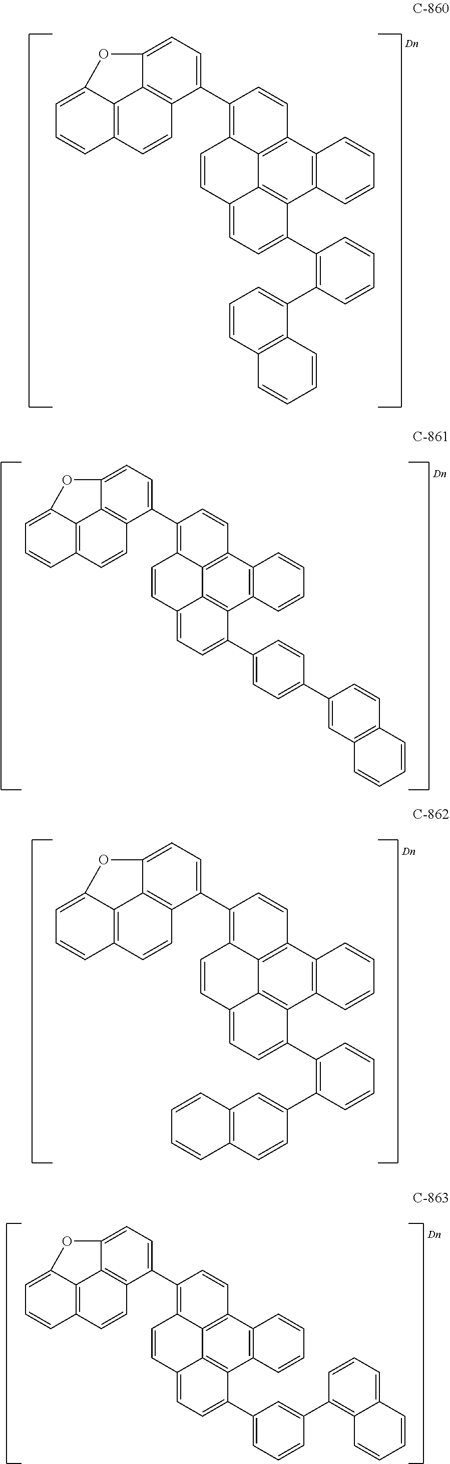

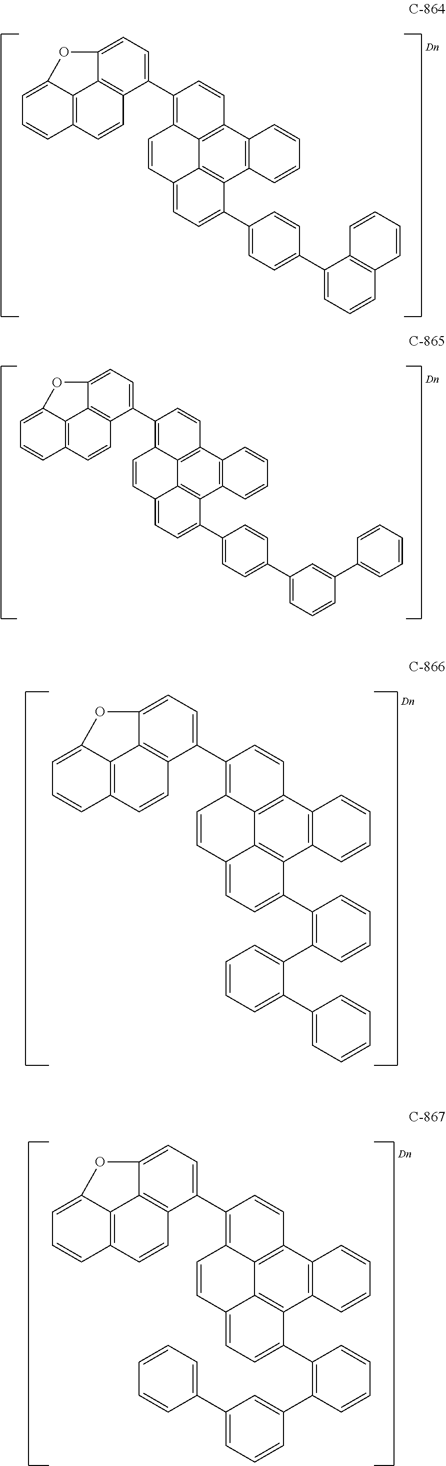

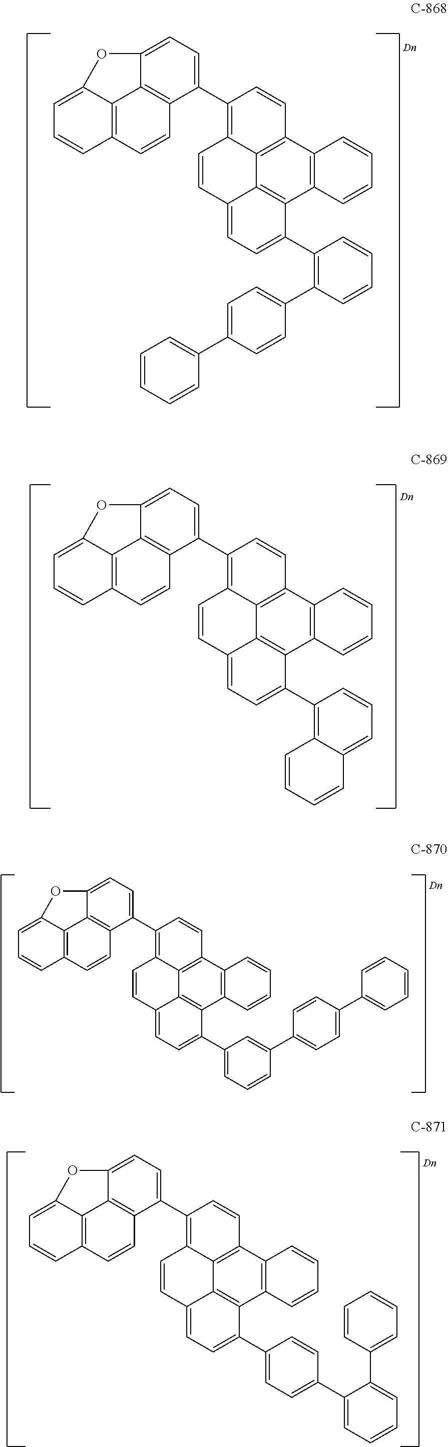

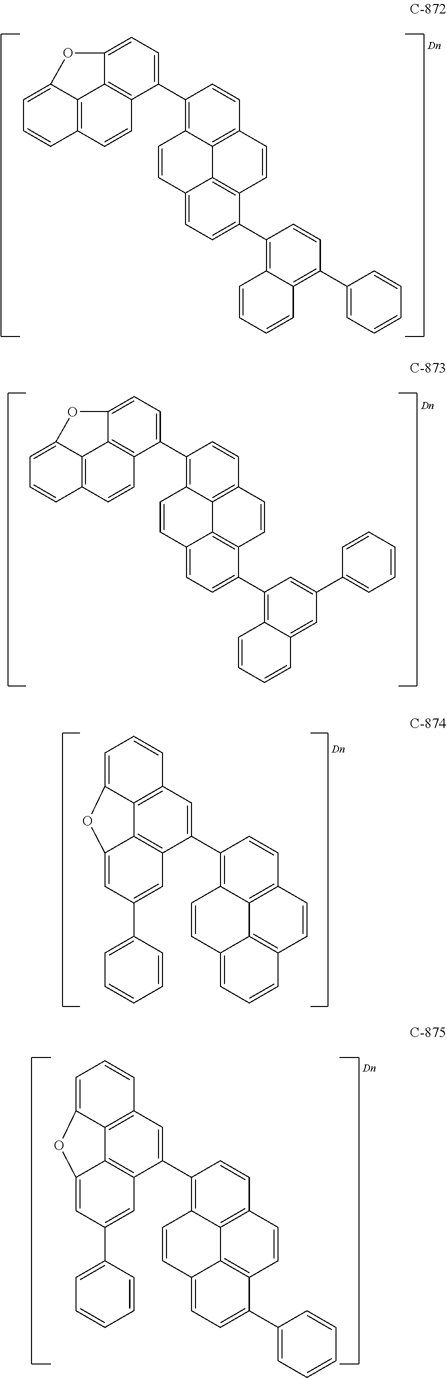

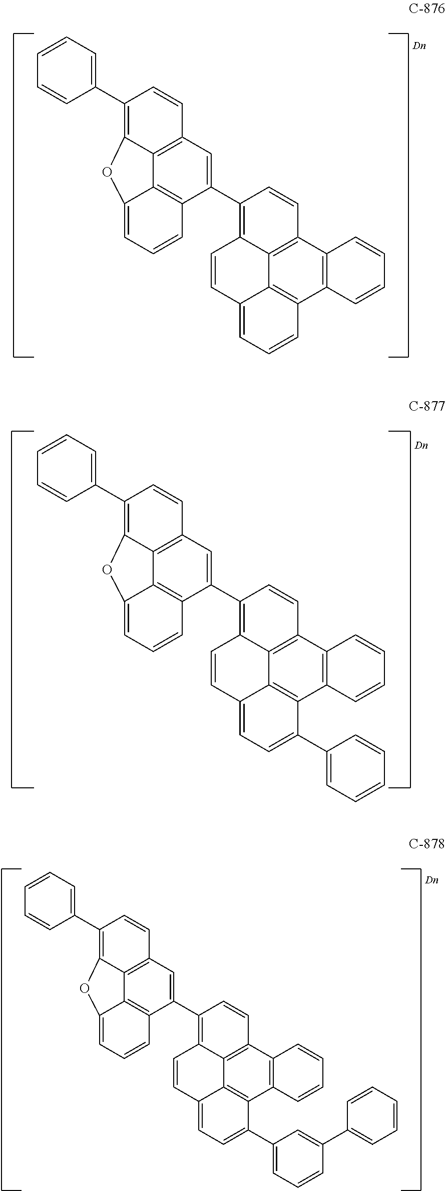

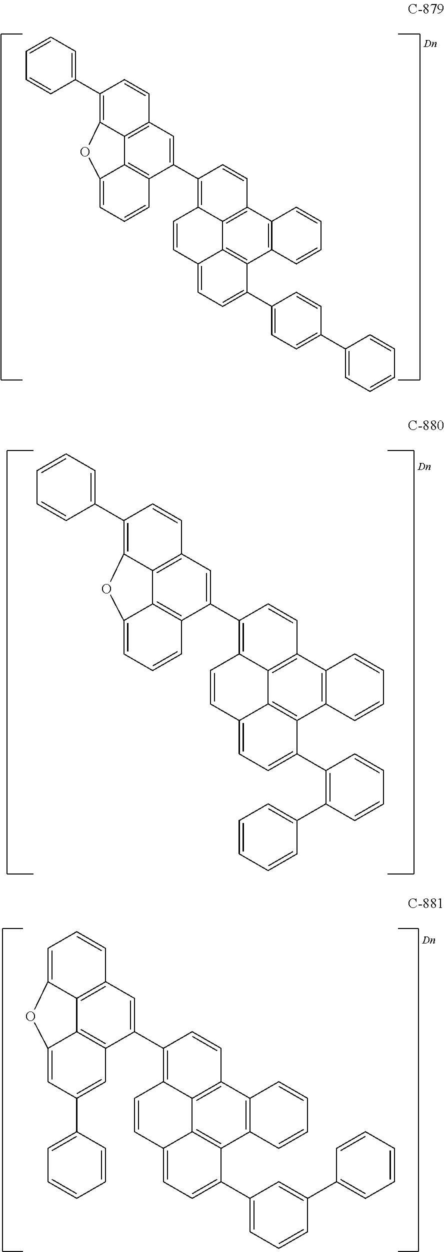

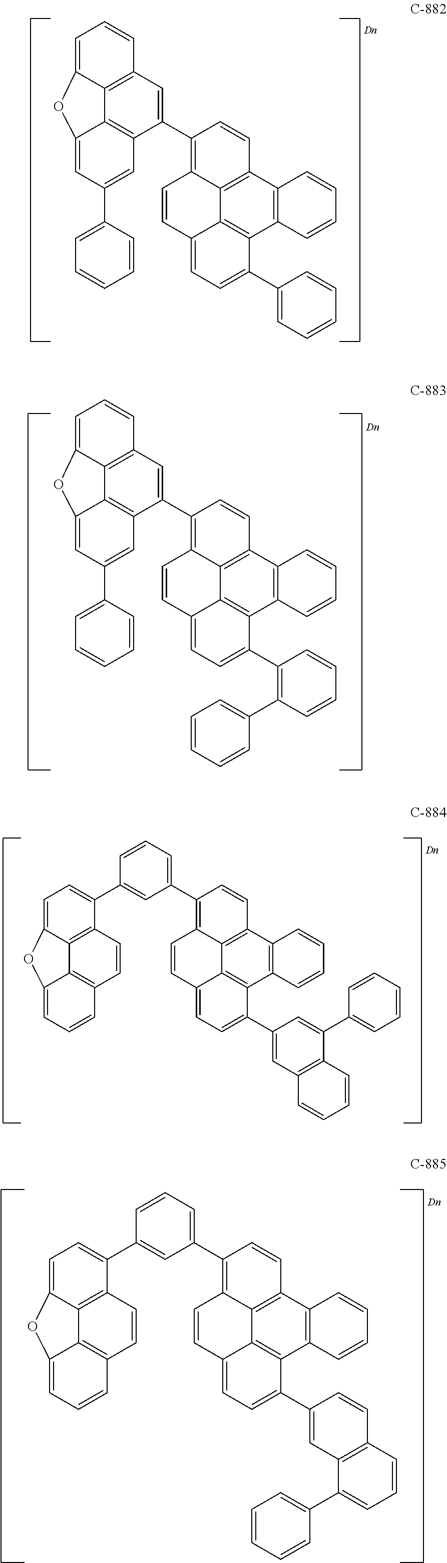

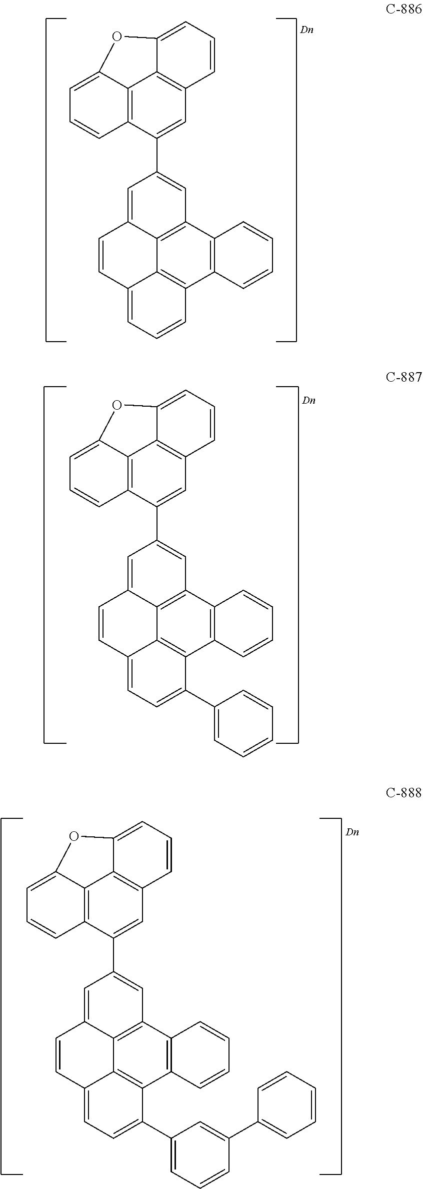

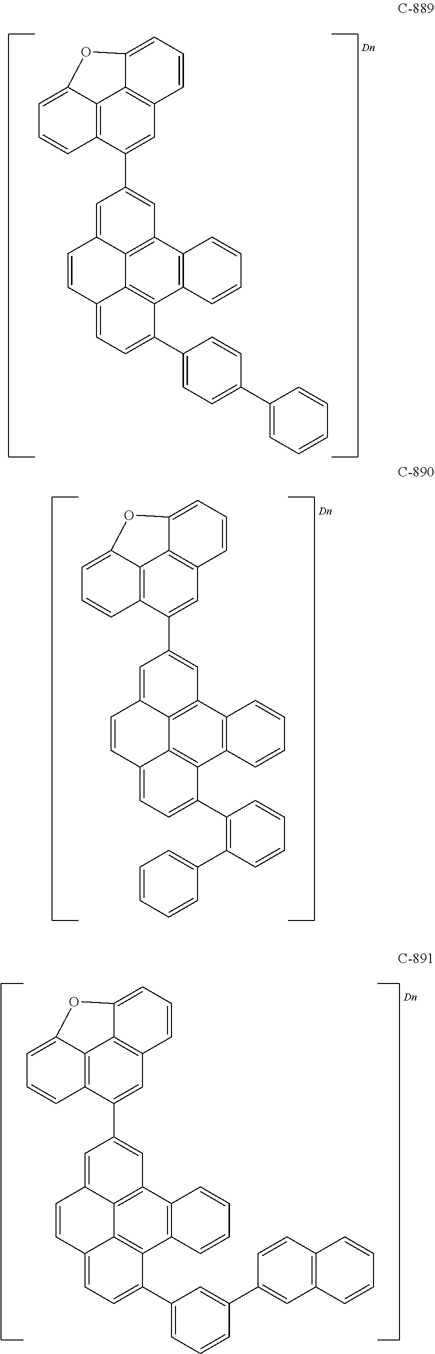

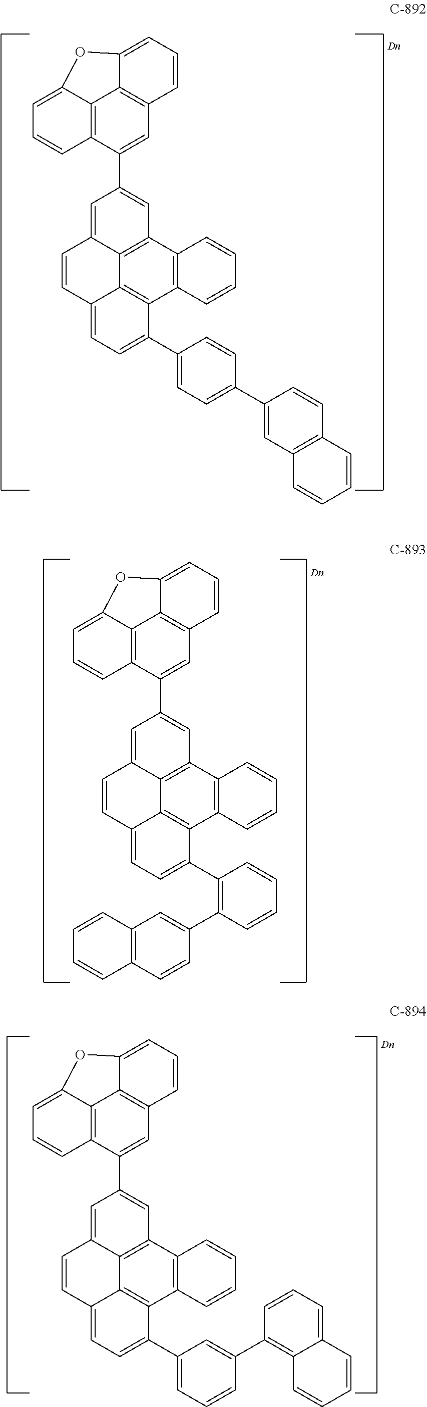

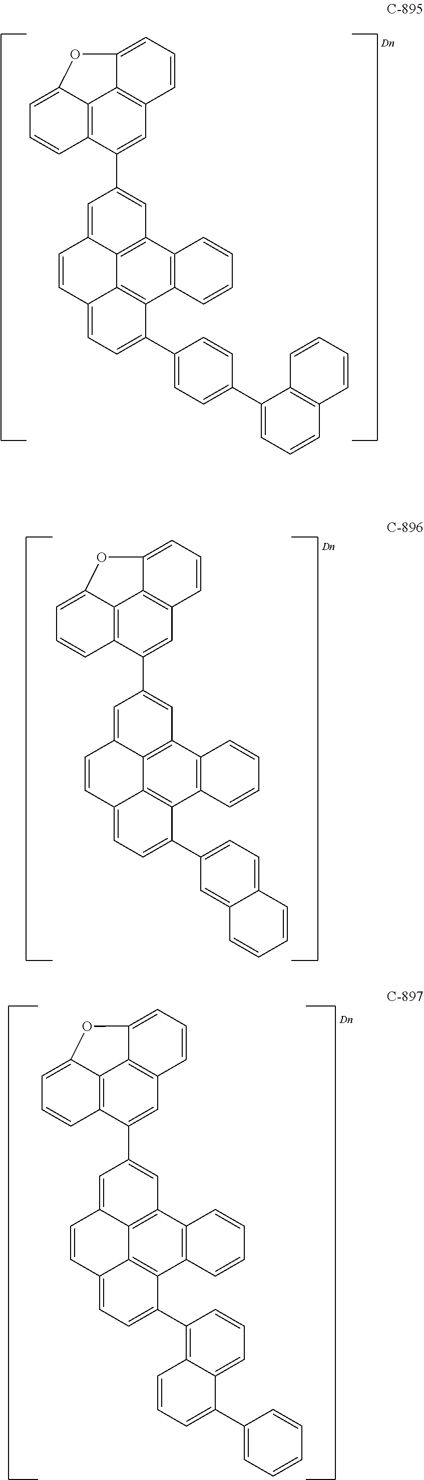

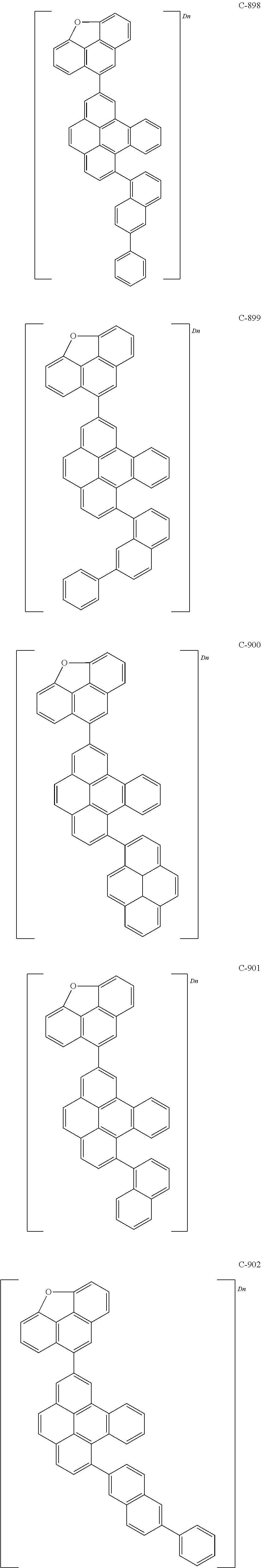

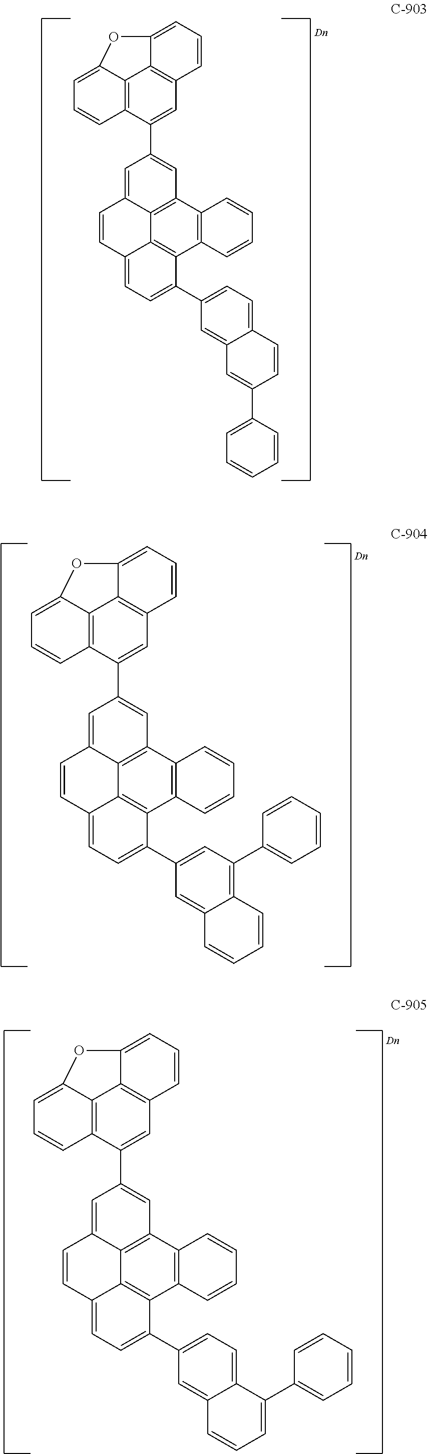

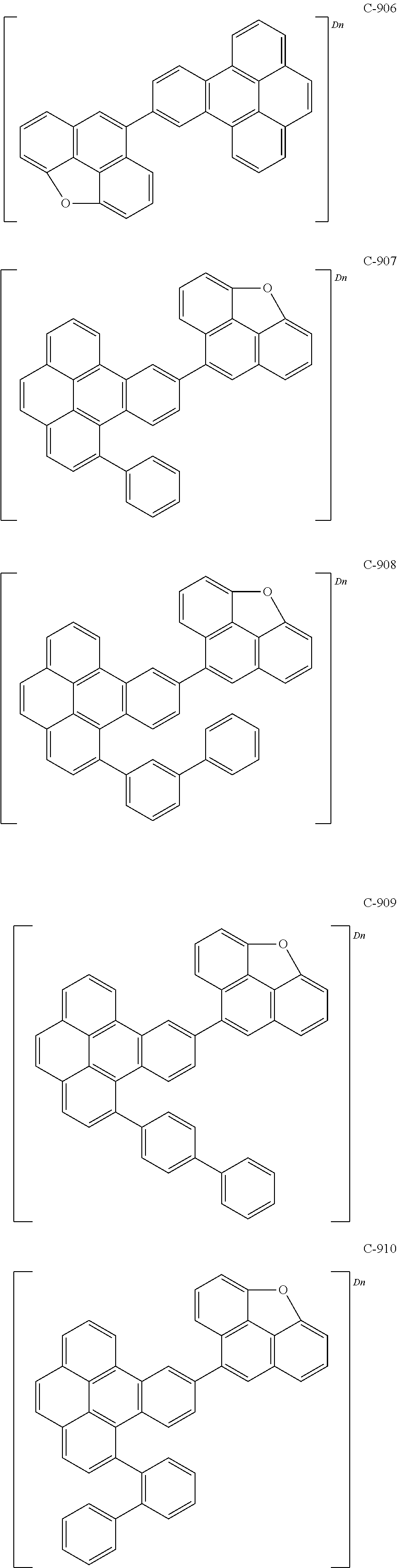

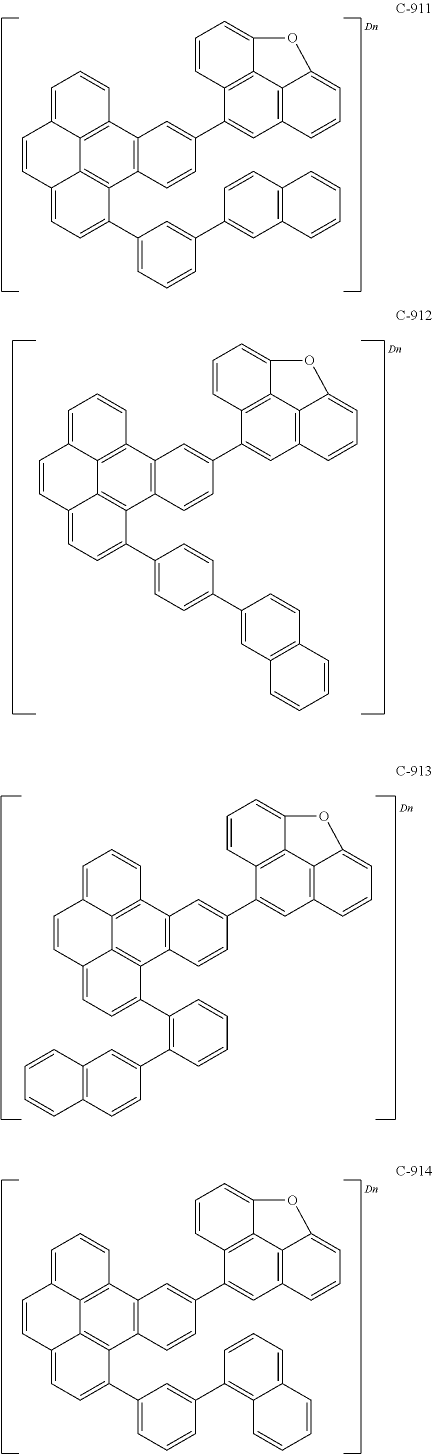

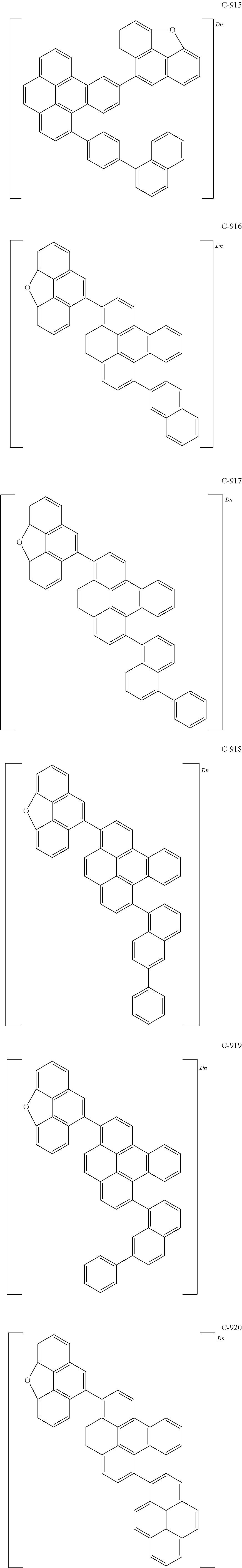

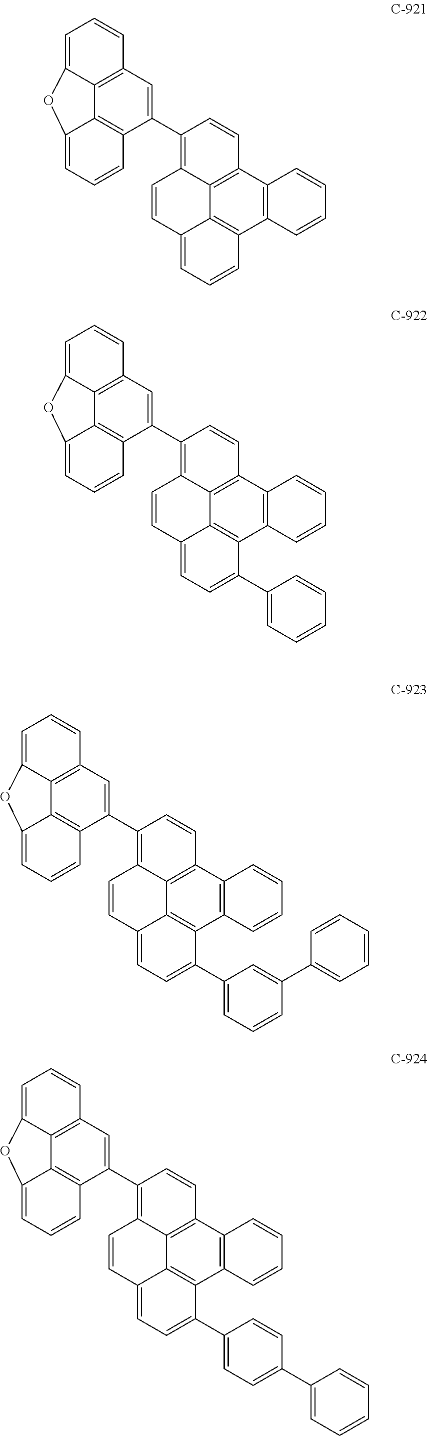 [SMILES: c1cc2cc(-c3ccc4c5ccccc5c5cccc6ccc3c4c65)c3cccc4oc(c1)c2c43 ; c1ccc(-c2ccc3ccc4c(-c5cc6cccc7oc8cccc5c8c67)ccc5c6ccccc6c2c3c45)cc1 ; c1ccc(-c2cccc(-c3ccc4ccc5c(-c6cc7cccc8oc9cccc6c9c78)ccc6c7ccccc7c3c4c56)c2)cc1 ; c1ccc(-c2ccc(-c3ccc4ccc5c(-c6cc7cccc8oc9cccc6c9c78)ccc6c7ccccc7c3c4c56)cc2)cc1]

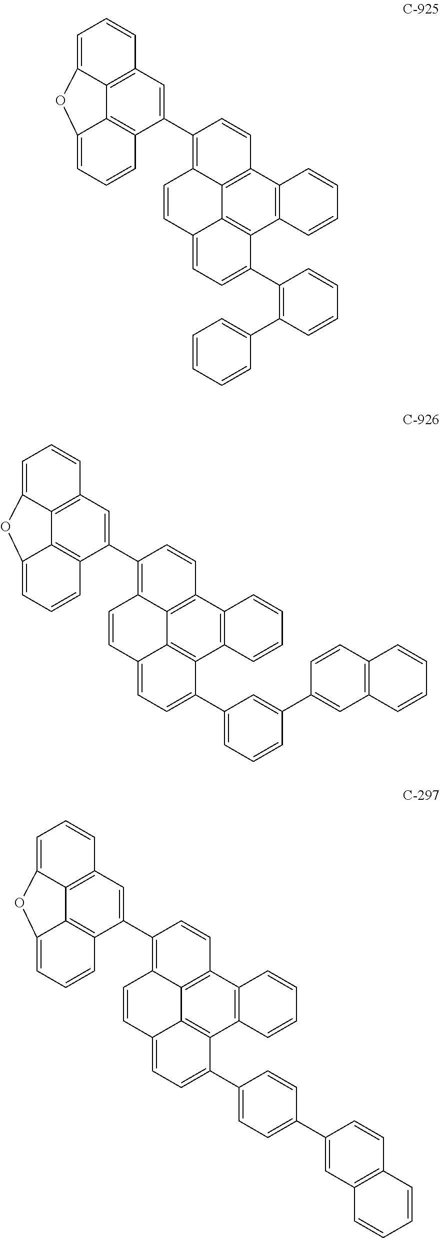 [SMILES: c1ccc(-c2ccccc2-c2ccc3ccc4c(-c5cc6cccc7oc8cccc5c8c67)ccc5c6ccccc6c2c3c45)cc1 ; c1cc(-c2ccc3ccccc3c2)cc(-c2ccc3ccc4c(-c5cc6cccc7oc8cccc5c8c67)ccc5c6ccccc6c2c3c45)c1 ; c1ccc2cc(-c3ccc(-c4ccc5ccc6c(-c7cc8cccc9oc%10cccc7c%10c89)ccc7c8ccccc8c4c5c67)cc3)ccc2c1]

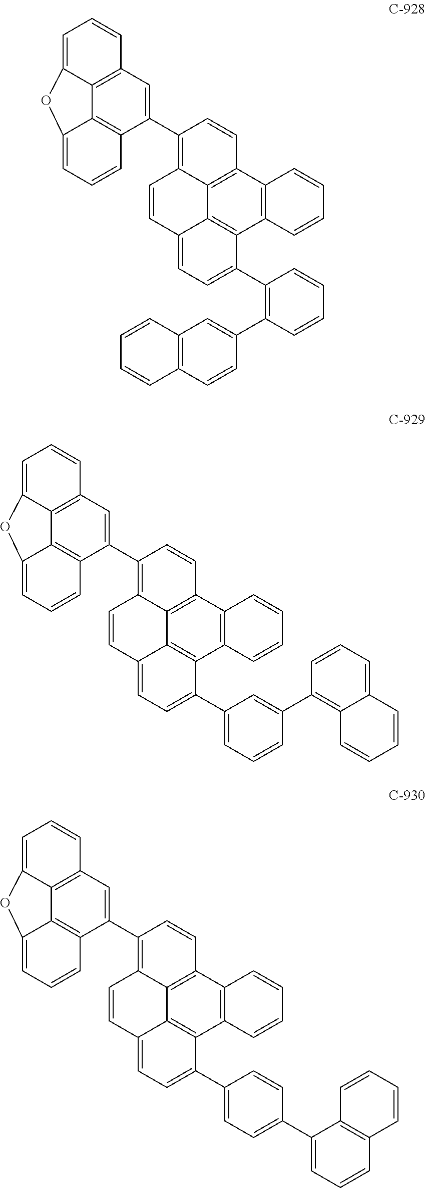 [SMILES: c1ccc(-c2ccc3ccc4c(-c5cc6cccc7oc8cccc5c8c67)ccc5c6ccccc6c2c3c45)c(-c2ccc3ccccc3c2)c1 ; c1cc(-c2cccc3ccccc23)cc(-c2ccc3ccc4c(-c5cc6cccc7oc8cccc5c8c67)ccc5c6ccccc6c2c3c45)c1 ; c1ccc2c(-c3ccc(-c4ccc5ccc6c(-c7cc8cccc9oc%10cccc7c%10c89)ccc7c8ccccc8c4c5c67)cc3)cccc2c1]

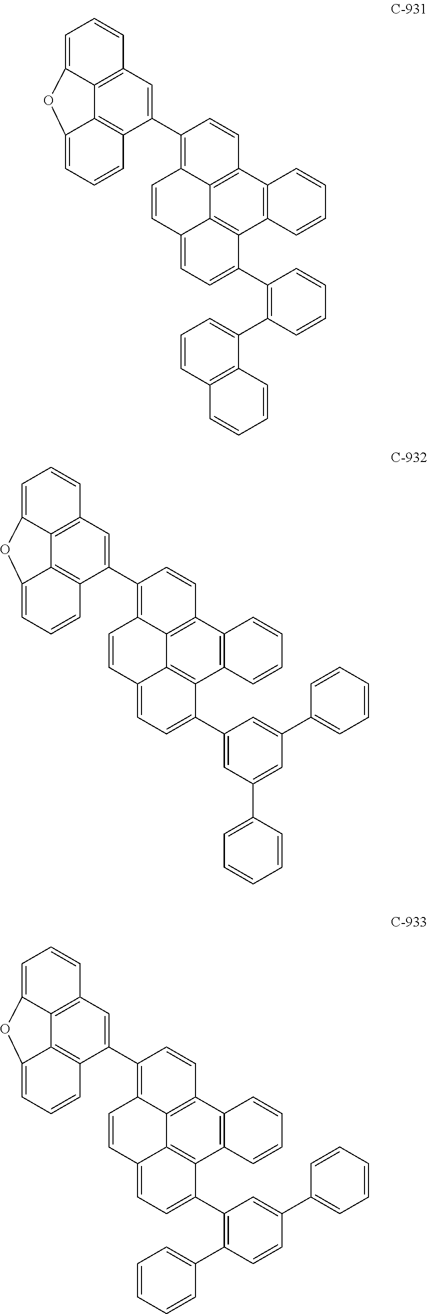 [SMILES: c1ccc(-c2ccc3ccc4c(-c5cc6cccc7oc8cccc5c8c67)ccc5c6ccccc6c2c3c45)c(-c2cccc3ccccc23)c1 ; c1ccc(-c2cc(-c3ccccc3)cc(-c3ccc4ccc5c(-c6cc7cccc8oc9cccc6c9c78)ccc6c7ccccc7c3c4c56)c2)cc1 ; c1ccc(-c2ccc(-c3ccccc3)c(-c3ccc4ccc5c(-c6cc7cccc8oc9cccc6c9c78)ccc6c7ccccc7c3c4c56)c2)cc1]

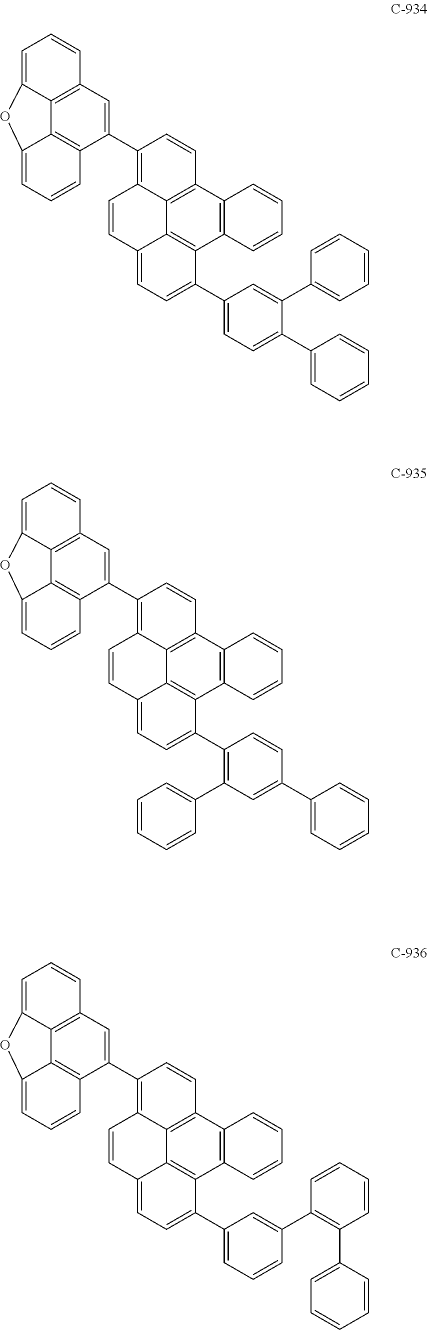 [SMILES: c1ccc(-c2ccc(-c3ccc4ccc5c(-c6cc7cccc8oc9cccc6c9c78)ccc6c7ccccc7c3c4c56)cc2-c2ccccc2)cc1 ; c1ccc(-c2ccc(-c3ccc4ccc5c(-c6cc7cccc8oc9cccc6c9c78)ccc6c7ccccc7c3c4c56)c(-c3ccccc3)c2)cc1 ; c1ccc(-c2ccccc2-c2cccc(-c3ccc4ccc5c(-c6cc7cccc8oc9cccc6c9c78)ccc6c7ccccc7c3c4c56)c2)cc1]

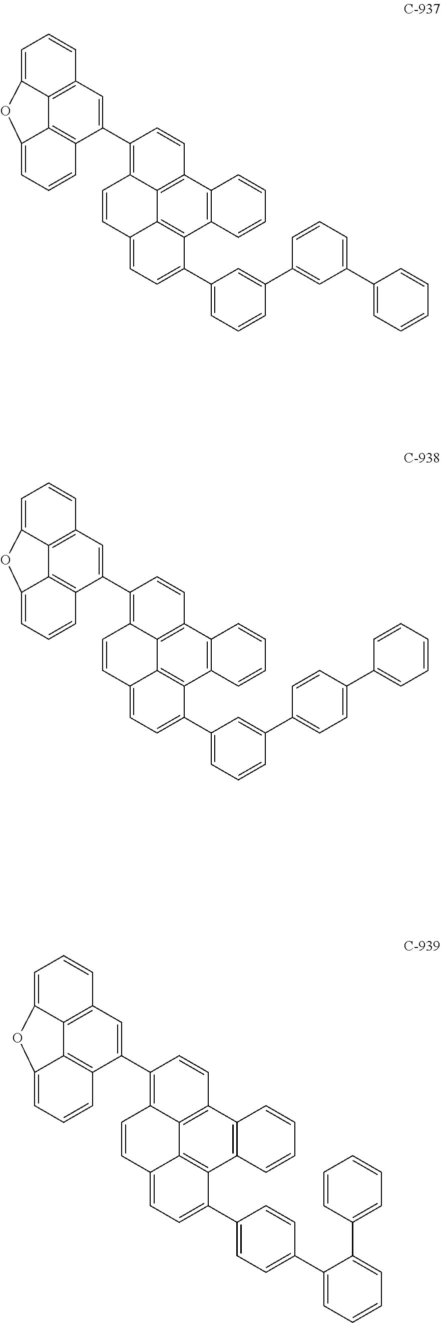 [SMILES: c1ccc(-c2cccc(-c3cccc(-c4ccc5ccc6c(-c7cc8cccc9oc%10cccc7c%10c89)ccc7c8ccccc8c4c5c67)c3)c2)cc1 ; c1ccc(-c2ccc(-c3cccc(-c4ccc5ccc6c(-c7cc8cccc9oc%10cccc7c%10c89)ccc7c8ccccc8c4c5c67)c3)cc2)cc1 ; c1ccc(-c2ccccc2-c2ccc(-c3ccc4ccc5c(-c6cc7cccc8oc9cccc6c9c78)ccc6c7ccccc7c3c4c56)cc2)cc1]

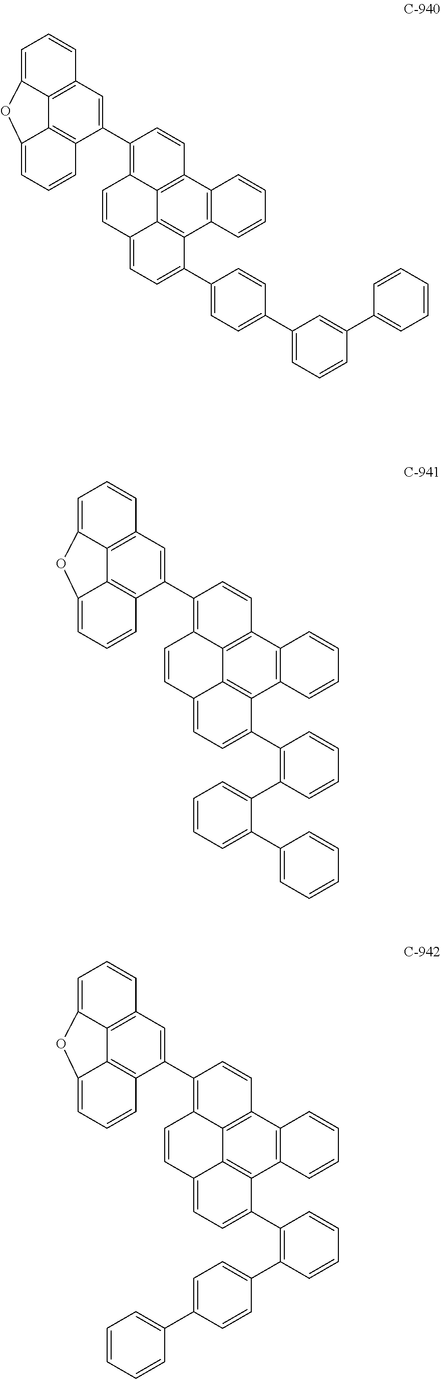 [SMILES: c1ccc(-c2cccc(-c3ccc(-c4ccc5ccc6c(-c7cc8cccc9oc%10cccc7c%10c89)ccc7c8ccccc8c4c5c67)cc3)c2)cc1 ; c1ccc(-c2ccccc2-c2ccccc2-c2ccc3ccc4c(-c5cc6cccc7oc8cccc5c8c67)ccc5c6ccccc6c2c3c45)cc1 ; c1ccc(-c2ccc(-c3ccccc3-c3ccc4ccc5c(-c6cc7cccc8oc9cccc6c9c78)ccc6c7ccccc7c3c4c56)cc2)cc1]

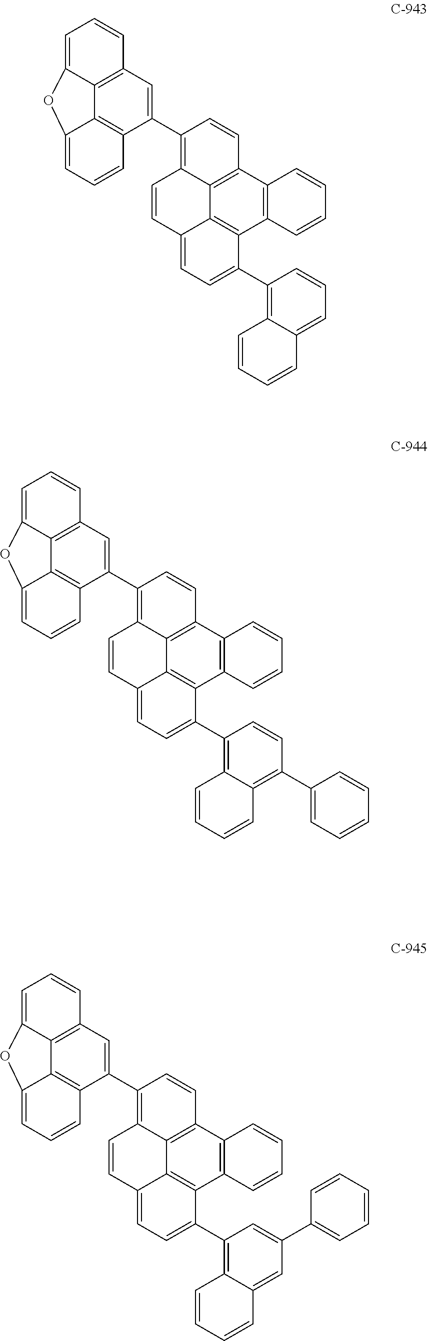 [SMILES: c1ccc2c(-c3ccc4ccc5c(-c6cc7cccc8oc9cccc6c9c78)ccc6c7ccccc7c3c4c56)cccc2c1 ; c1ccc(-c2ccc(-c3ccc4ccc5c(-c6cc7cccc8oc9cccc6c9c78)ccc6c7ccccc7c3c4c56)c3ccccc23)cc1 ; c1ccc(-c2cc(-c3ccc4ccc5c(-c6cc7cccc8oc9cccc6c9c78)ccc6c7ccccc7c3c4c56)c3ccccc3c2)cc1]

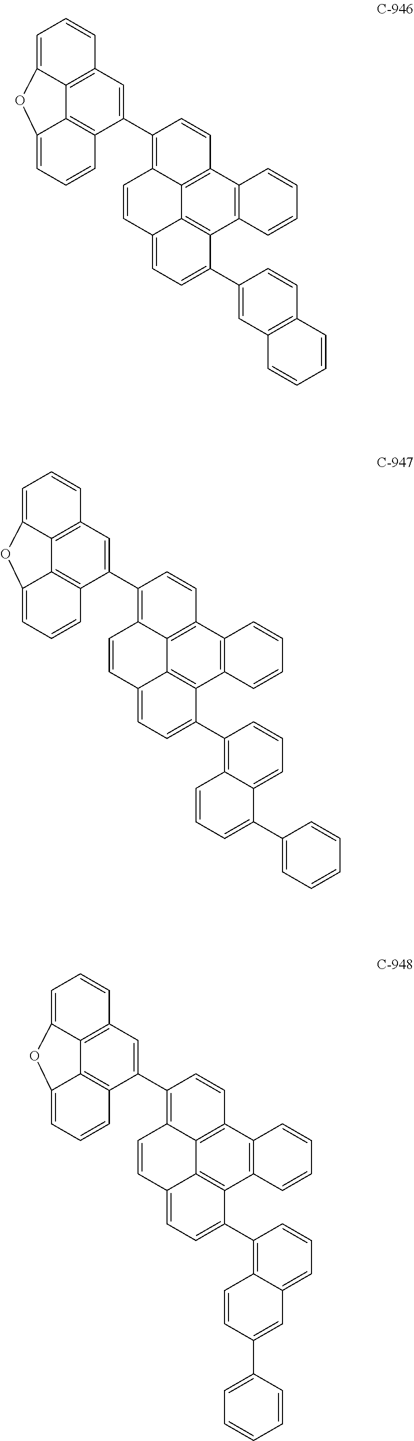 [SMILES: c1ccc2cc(-c3ccc4ccc5c(-c6cc7cccc8oc9cccc6c9c78)ccc6c7ccccc7c3c4c56)ccc2c1 ; c1ccc(-c2cccc3c(-c4ccc5ccc6c(-c7cc8cccc9oc%10cccc7c%10c89)ccc7c8ccccc8c4c5c67)cccc23)cc1 ; c1ccc(-c2ccc3c(-c4ccc5ccc6c(-c7cc8cccc9oc%10cccc7c%10c89)ccc7c8ccccc8c4c5c67)cccc3c2)cc1]

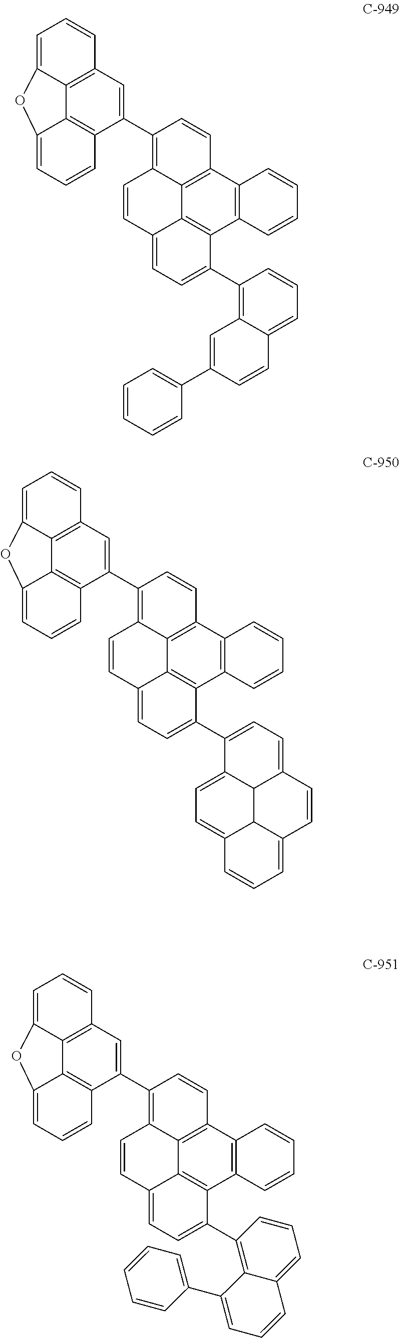 [SMILES: c1ccc(-c2ccc3cccc(-c4ccc5ccc6c(-c7cc8cccc9oc%10cccc7c%10c89)ccc7c8ccccc8c4c5c67)c3c2)cc1 ; C1=CC2=CC=C3C(c4ccc5ccc6c(-c7cc8cccc9oc%10cccc7c%10c89)ccc7c8ccccc8c4c5c67)=CC=C4C=CC(=C1)C2C43 ; c1ccc(-c2cccc3cccc(-c4ccc5ccc6c(-c7cc8cccc9oc%10cccc7c%10c89)ccc7c8ccccc8c4c5c67)c23)cc1]

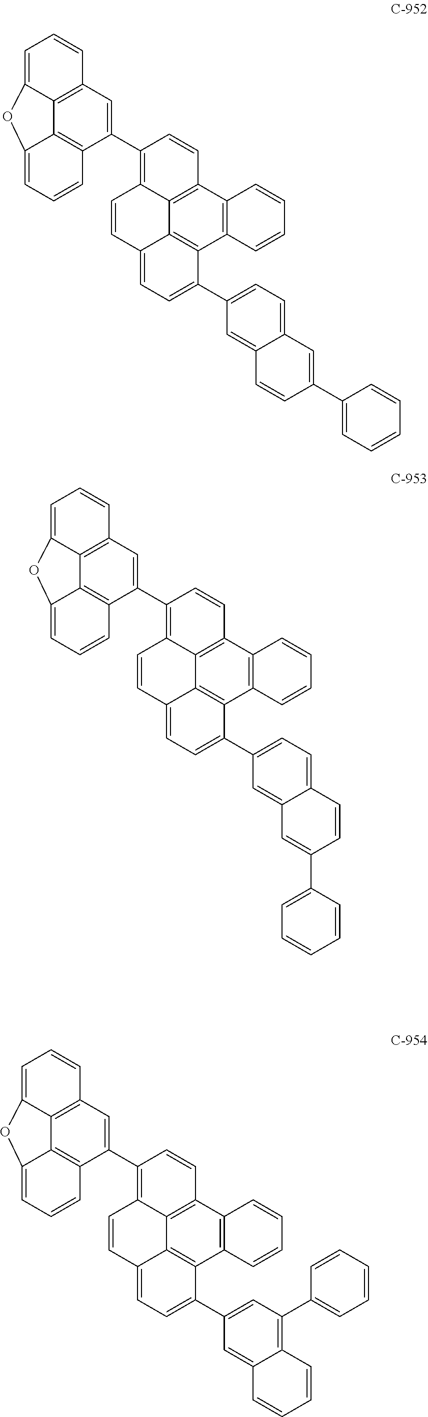 [SMILES: c1ccc(-c2ccc3cc(-c4ccc5ccc6c(-c7cc8cccc9oc%10cccc7c%10c89)ccc7c8ccccc8c4c5c67)ccc3c2)cc1 ; c1ccc(-c2ccc3ccc(-c4ccc5ccc6c(-c7cc8cccc9oc%10cccc7c%10c89)ccc7c8ccccc8c4c5c67)cc3c2)cc1 ; c1ccc(-c2cc(-c3ccc4ccc5c(-c6cc7cccc8oc9cccc6c9c78)ccc6c7ccccc7c3c4c56)cc3ccccc23)cc1]

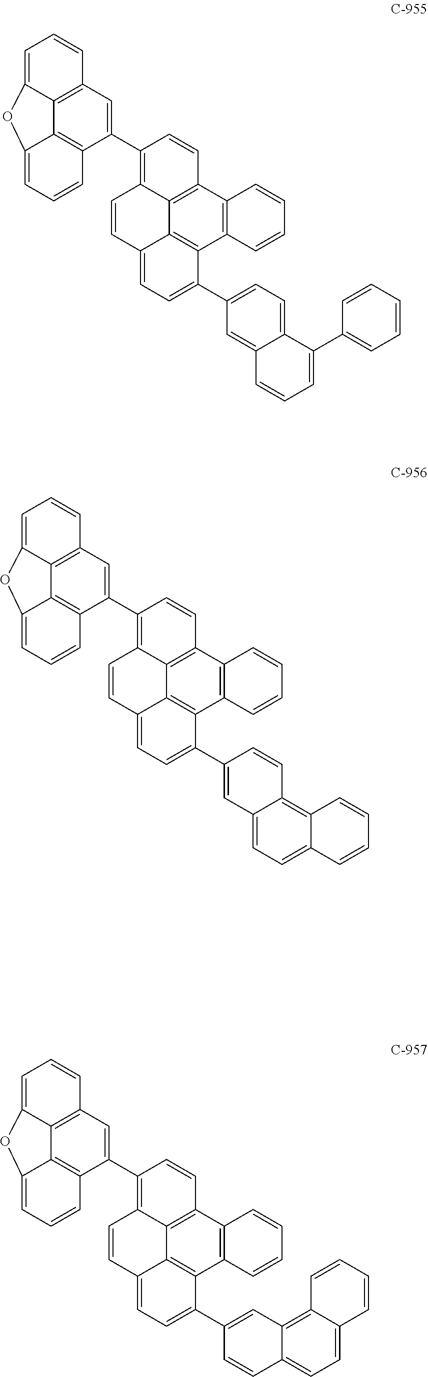 [SMILES: c1ccc(-c2cccc3cc(-c4ccc5ccc6c(-c7cc8cccc9oc%10cccc7c%10c89)ccc7c8ccccc8c4c5c67)ccc23)cc1 ; c1ccc2c(c1)ccc1cc(-c3ccc4ccc5c(-c6cc7cccc8oc9cccc6c9c78)ccc6c7ccccc7c3c4c56)ccc12 ; c1ccc2c(c1)ccc1ccc(-c3ccc4ccc5c(-c6cc7cccc8oc9cccc6c9c78)ccc6c7ccccc7c3c4c56)cc12]

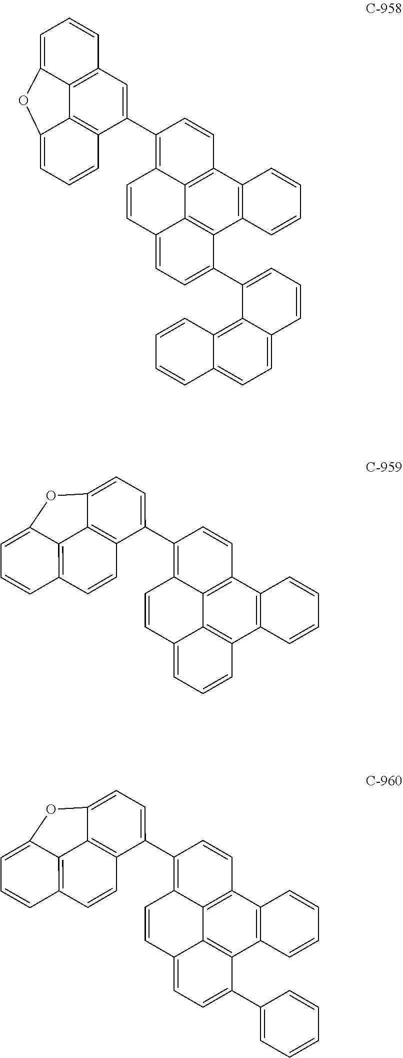 [SMILES: c1ccc2c(c1)ccc1cccc(-c3ccc4ccc5c(-c6cc7cccc8oc9cccc6c9c78)ccc6c7ccccc7c3c4c56)c12 ; c1cc2ccc3c(-c4ccc5c6ccccc6c6cccc7ccc4c5c76)ccc4oc(c1)c2c43 ; c1ccc(-c2ccc3ccc4c(-c5ccc6oc7cccc8ccc5c6c87)ccc5c6ccccc6c2c3c45)cc1]

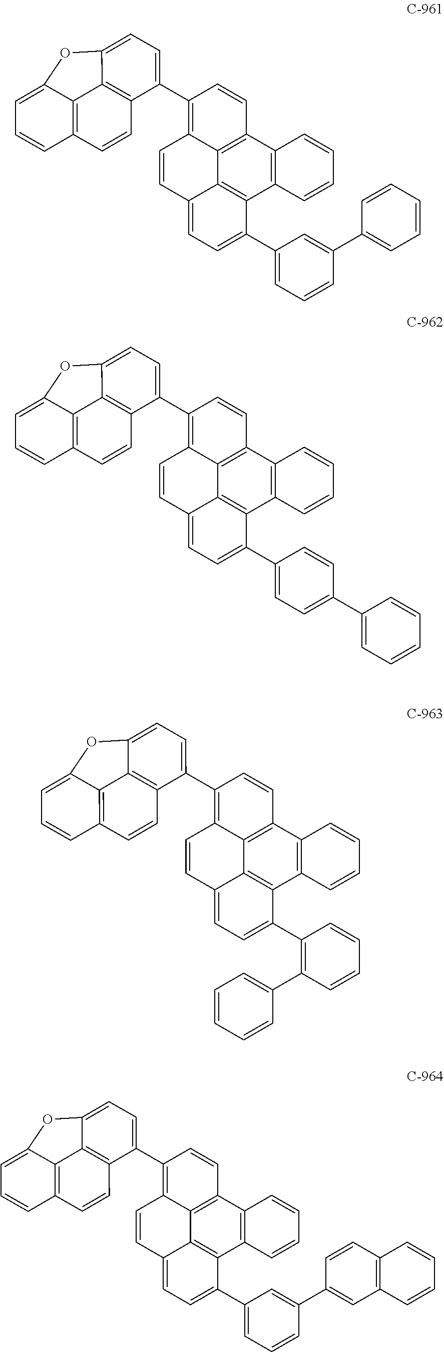 [SMILES: c1ccc(-c2cccc(-c3ccc4ccc5c(-c6ccc7oc8cccc9ccc6c7c98)ccc6c7ccccc7c3c4c56)c2)cc1 ; c1ccc(-c2ccc(-c3ccc4ccc5c(-c6ccc7oc8cccc9ccc6c7c98)ccc6c7ccccc7c3c4c56)cc2)cc1 ; c1ccc(-c2ccccc2-c2ccc3ccc4c(-c5ccc6oc7cccc8ccc5c6c87)ccc5c6ccccc6c2c3c45)cc1 ; c1cc(-c2ccc3ccccc3c2)cc(-c2ccc3ccc4c(-c5ccc6oc7cccc8ccc5c6c87)ccc5c6ccccc6c2c3c45)c1]

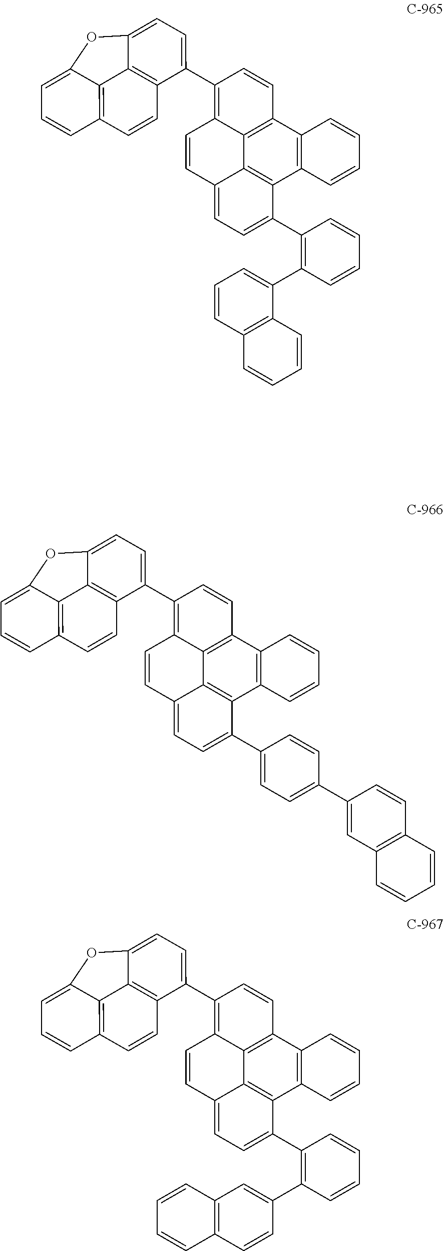 [SMILES: c1ccc(-c2ccc3ccc4c(-c5ccc6oc7cccc8ccc5c6c87)ccc5c6ccccc6c2c3c45)c(-c2cccc3ccccc23)c1 ; c1ccc2cc(-c3ccc(-c4ccc5ccc6c(-c7ccc8oc9cccc%10ccc7c8c%109)ccc7c8ccccc8c4c5c67)cc3)ccc2c1 ; c1ccc(-c2ccc3ccc4c(-c5ccc6oc7cccc8ccc5c6c87)ccc5c6ccccc6c2c3c45)c(-c2ccc3ccccc3c2)c1]

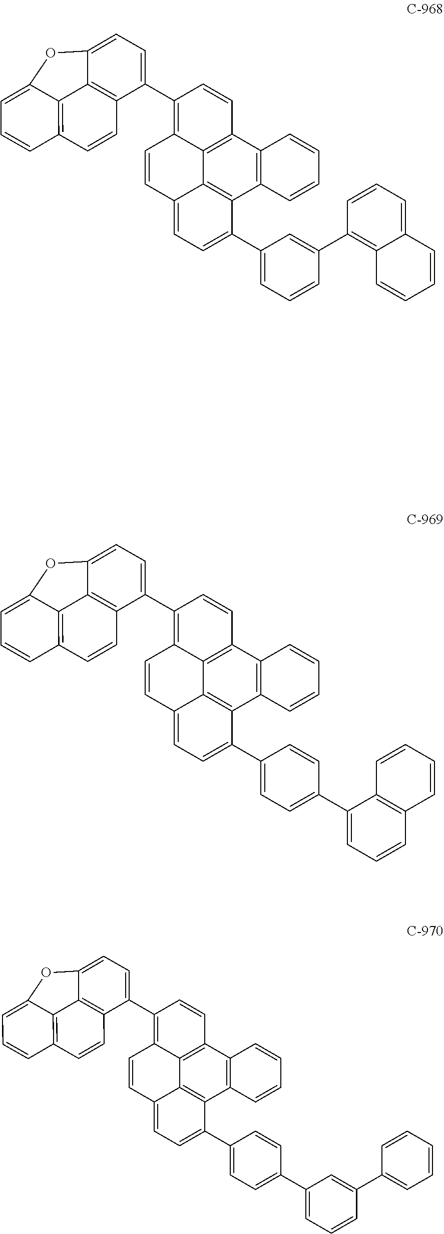 [SMILES: c1cc(-c2cccc3ccccc23)cc(-c2ccc3ccc4c(-c5ccc6oc7cccc8ccc5c6c87)ccc5c6ccccc6c2c3c45)c1 ; c1ccc2c(-c3ccc(-c4ccc5ccc6c(-c7ccc8oc9cccc%10ccc7c8c%109)ccc7c8ccccc8c4c5c67)cc3)cccc2c1 ; c1ccc(-c2cccc(-c3ccc(-c4ccc5ccc6c(-c7ccc8oc9cccc%10ccc7c8c%109)ccc7c8ccccc8c4c5c67)cc3)c2)cc1]

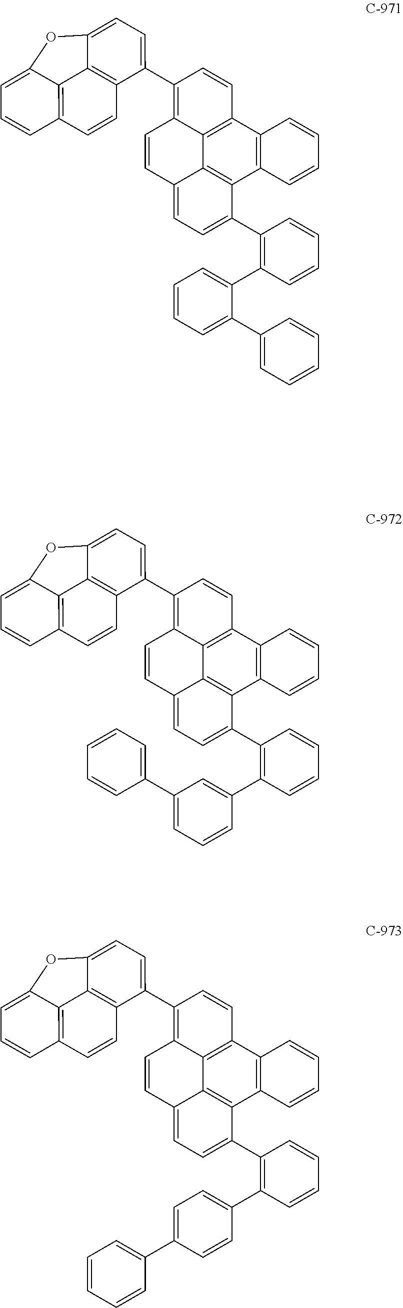 [SMILES: c1ccc(-c2ccccc2-c2ccccc2-c2ccc3ccc4c(-c5ccc6oc7cccc8ccc5c6c87)ccc5c6ccccc6c2c3c45)cc1 ; c1ccc(-c2cccc(-c3ccccc3-c3ccc4ccc5c(-c6ccc7oc8cccc9ccc6c7c98)ccc6c7ccccc7c3c4c56)c2)cc1 ; c1ccc(-c2ccc(-c3ccccc3-c3ccc4ccc5c(-c6ccc7oc8cccc9ccc6c7c98)ccc6c7ccccc7c3c4c56)cc2)cc1]

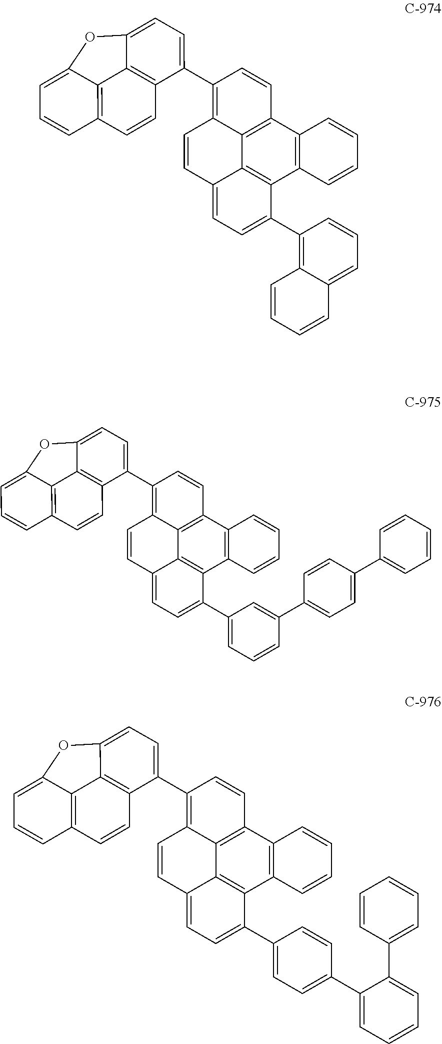 [SMILES: c1ccc2c(-c3ccc4ccc5c(-c6ccc7oc8cccc9ccc6c7c98)ccc6c7ccccc7c3c4c56)cccc2c1 ; c1ccc(-c2ccc(-c3cccc(-c4ccc5ccc6c(-c7ccc8oc9cccc%10ccc7c8c%109)ccc7c8ccccc8c4c5c67)c3)cc2)cc1 ; c1ccc(-c2ccccc2-c2ccc(-c3ccc4ccc5c(-c6ccc7oc8cccc9ccc6c7c98)ccc6c7ccccc7c3c4c56)cc2)cc1]

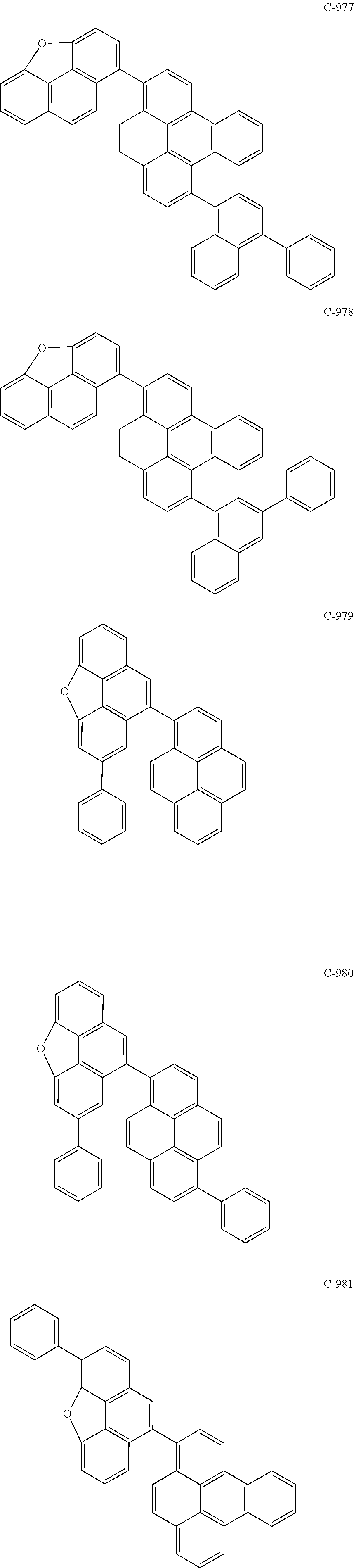 [SMILES: c1ccc(-c2ccc(-c3ccc4ccc5c(-c6ccc7oc8cccc9ccc6c7c98)ccc6c7ccccc7c3c4c56)c3ccccc23)cc1 ; c1ccc(-c2cc(-c3ccc4ccc5c(-c6ccc7oc8cccc9ccc6c7c98)ccc6c7ccccc7c3c4c56)c3ccccc3c2)cc1 ; c1ccc(-c2cc3oc4cccc5cc(-c6ccc7ccc8cccc9ccc6c7c89)c(c2)c3c54)cc1 ; c1ccc(-c2cc3oc4cccc5cc(-c6ccc7ccc8c(-c9ccccc9)ccc9ccc6c7c98)c(c2)c3c54)cc1 ; c1ccc(-c2ccc3cc(-c4ccc5c6ccccc6c6cccc7ccc4c5c76)c4cccc5oc2c3c54)cc1]

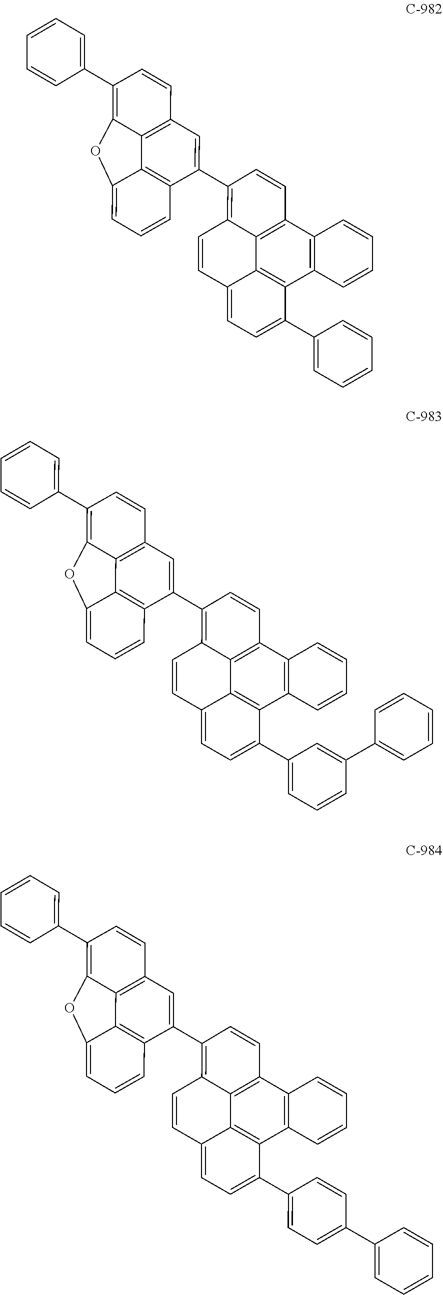 [SMILES: c1ccc(-c2ccc3cc(-c4ccc5c6ccccc6c6c(-c7ccccc7)ccc7ccc4c5c76)c4cccc5oc2c3c54)cc1 ; c1ccc(-c2cccc(-c3ccc4ccc5c(-c6cc7ccc(-c8ccccc8)c8oc9cccc6c9c78)ccc6c7ccccc7c3c4c56)c2)cc1 ; c1ccc(-c2ccc(-c3ccc4ccc5c(-c6cc7ccc(-c8ccccc8)c8oc9cccc6c9c78)ccc6c7ccccc7c3c4c56)cc2)cc1]

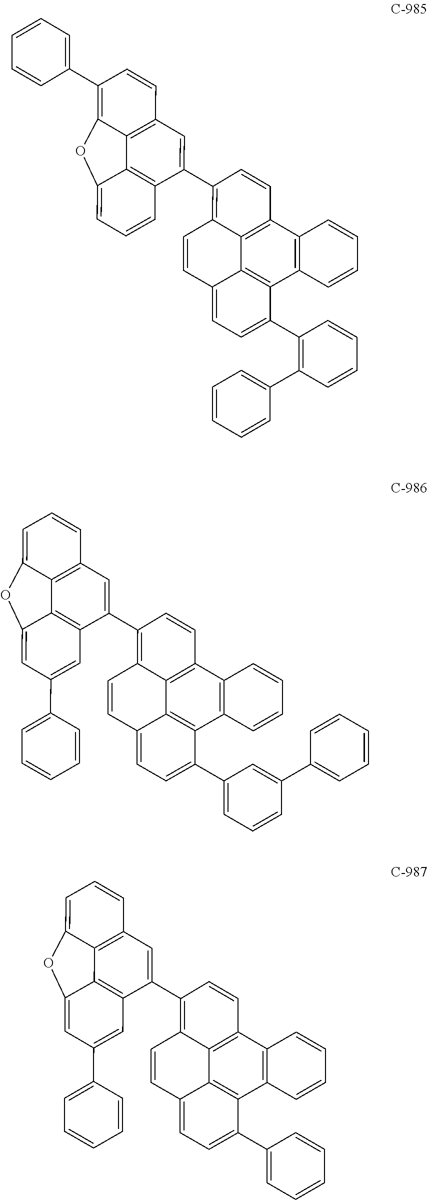 [SMILES: c1ccc(-c2ccccc2-c2ccc3ccc4c(-c5cc6ccc(-c7ccccc7)c7oc8cccc5c8c67)ccc5c6ccccc6c2c3c45)cc1 ; c1ccc(-c2cccc(-c3ccc4ccc5c(-c6cc7cccc8oc9cc(-c%10ccccc%10)cc6c9c78)ccc6c7ccccc7c3c4c56)c2)cc1 ; c1ccc(-c2cc3oc4cccc5cc(-c6ccc7c8ccccc8c8c(-c9ccccc9)ccc9ccc6c7c98)c(c2)c3c54)cc1]

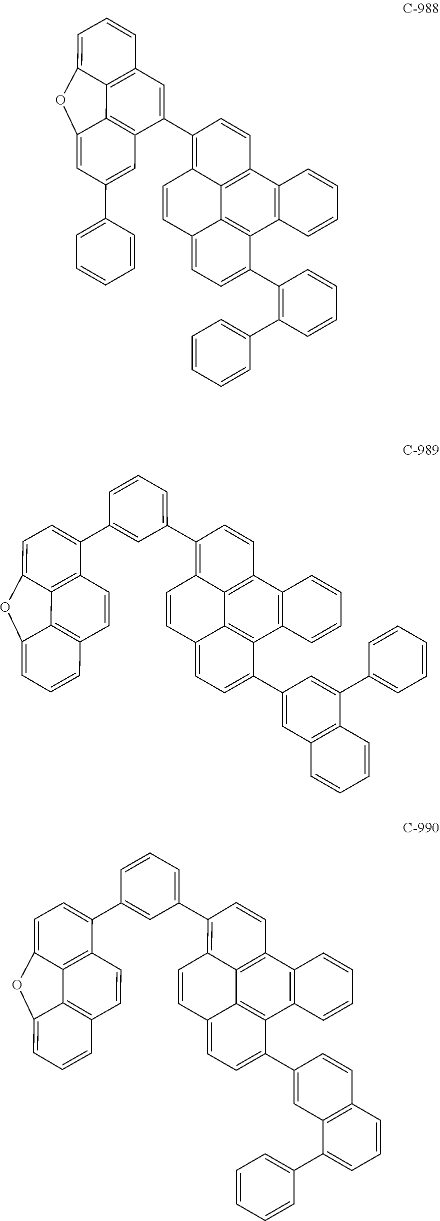 [SMILES: c1ccc(-c2cc3oc4cccc5cc(-c6ccc7c8ccccc8c8c(-c9ccccc9-c9ccccc9)ccc9ccc6c7c98)c(c2)c3c54)cc1 ; c1ccc(-c2cc(-c3ccc4ccc5c(-c6cccc(-c7ccc8oc9cccc%10ccc7c8c%109)c6)ccc6c7ccccc7c3c4c56)cc3ccccc23)cc1 ; c1ccc(-c2cccc3ccc(-c4ccc5ccc6c(-c7cccc(-c8ccc9oc%10cccc%11ccc8c9c%11%10)c7)ccc7c8ccccc8c4c5c67)cc23)cc1]

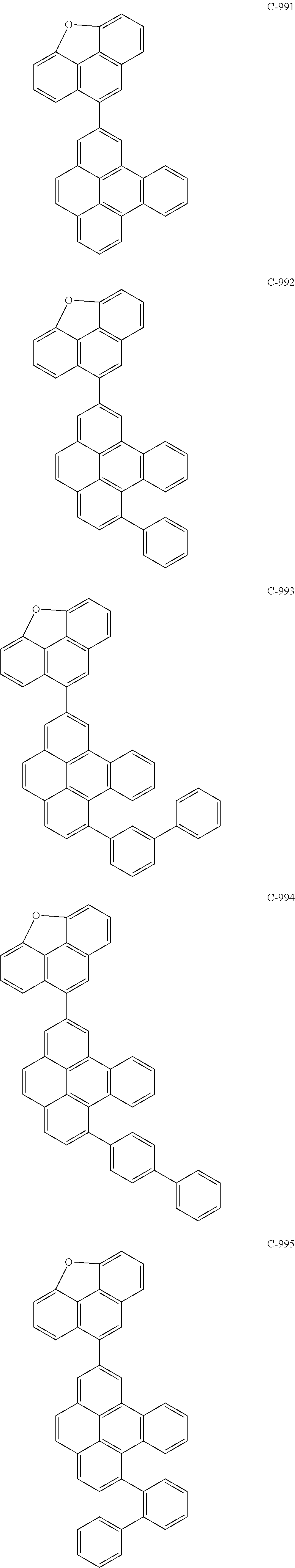 [SMILES: c1cc2cc(-c3cc4ccc5cccc6c7ccccc7c(c3)c4c56)c3cccc4oc(c1)c2c43 ; c1ccc(-c2ccc3ccc4cc(-c5cc6cccc7oc8cccc5c8c67)cc5c6ccccc6c2c3c45)cc1 ; c1ccc(-c2cccc(-c3ccc4ccc5cc(-c6cc7cccc8oc9cccc6c9c78)cc6c7ccccc7c3c4c56)c2)cc1 ; c1ccc(-c2ccc(-c3ccc4ccc5cc(-c6cc7cccc8oc9cccc6c9c78)cc6c7ccccc7c3c4c56)cc2)cc1 ; c1ccc(-c2ccccc2-c2ccc3ccc4cc(-c5cc6cccc7oc8cccc5c8c67)cc5c6ccccc6c2c3c45)cc1]

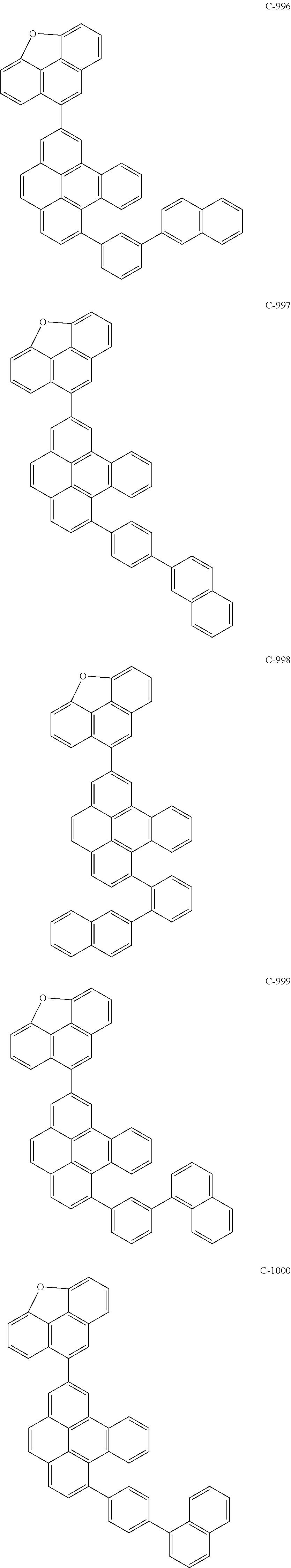 [SMILES: c1cc(-c2ccc3ccccc3c2)cc(-c2ccc3ccc4cc(-c5cc6cccc7oc8cccc5c8c67)cc5c6ccccc6c2c3c45)c1 ; c1ccc2cc(-c3ccc(-c4ccc5ccc6cc(-c7cc8cccc9oc%10cccc7c%10c89)cc7c8ccccc8c4c5c67)cc3)ccc2c1 ; c1ccc(-c2ccc3ccc4cc(-c5cc6cccc7oc8cccc5c8c67)cc5c6ccccc6c2c3c45)c(-c2ccc3ccccc3c2)c1 ; c1cc(-c2cccc3ccccc23)cc(-c2ccc3ccc4cc(-c5cc6cccc7oc8cccc5c8c67)cc5c6ccccc6c2c3c45)c1 ; c1ccc2c(-c3ccc(-c4ccc5ccc6cc(-c7cc8cccc9oc%10cccc7c%10c89)cc7c8ccccc8c4c5c67)cc3)cccc2c1]

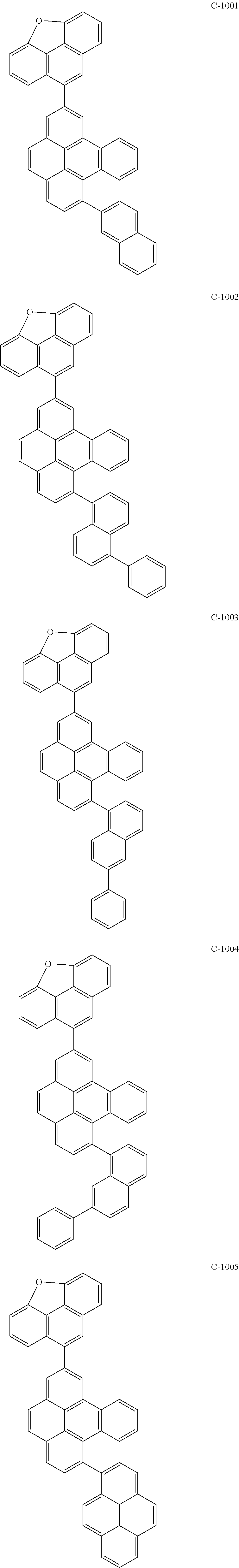 [SMILES: c1ccc2cc(-c3ccc4ccc5cc(-c6cc7cccc8oc9cccc6c9c78)cc6c7ccccc7c3c4c56)ccc2c1 ; c1ccc(-c2cccc3c(-c4ccc5ccc6cc(-c7cc8cccc9oc%10cccc7c%10c89)cc7c8ccccc8c4c5c67)cccc23)cc1 ; c1ccc(-c2ccc3c(-c4ccc5ccc6cc(-c7cc8cccc9oc%10cccc7c%10c89)cc7c8ccccc8c4c5c67)cccc3c2)cc1 ; c1ccc(-c2ccc3cccc(-c4ccc5ccc6cc(-c7cc8cccc9oc%10cccc7c%10c89)cc7c8ccccc8c4c5c67)c3c2)cc1 ; C1=CC2=CC=C3C(c4ccc5ccc6cc(-c7cc8cccc9oc%10cccc7c%10c89)cc7c8ccccc8c4c5c67)=CC=C4C=CC(=C1)C2C43]

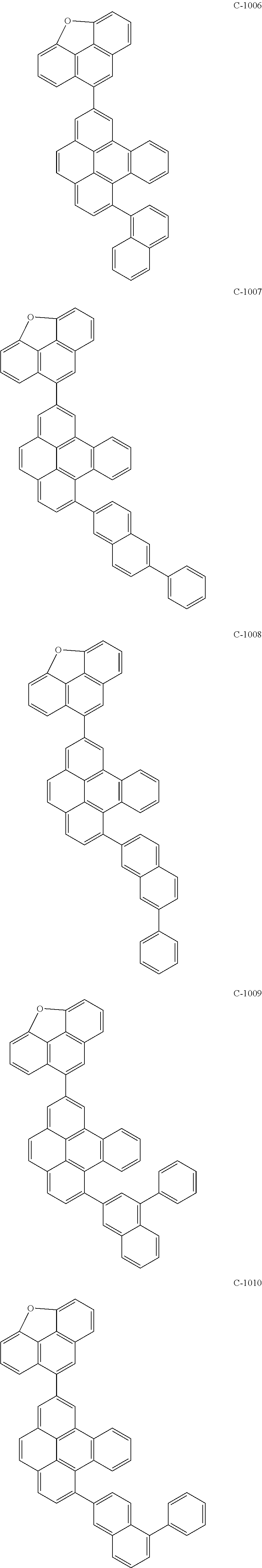 [SMILES: c1ccc2c(-c3ccc4ccc5cc(-c6cc7cccc8oc9cccc6c9c78)cc6c7ccccc7c3c4c56)cccc2c1 ; c1ccc(-c2ccc3cc(-c4ccc5ccc6cc(-c7cc8cccc9oc%10cccc7c%10c89)cc7c8ccccc8c4c5c67)ccc3c2)cc1 ; c1ccc(-c2ccc3ccc(-c4ccc5ccc6cc(-c7cc8cccc9oc%10cccc7c%10c89)cc7c8ccccc8c4c5c67)cc3c2)cc1 ; c1ccc(-c2cc(-c3ccc4ccc5cc(-c6cc7cccc8oc9cccc6c9c78)cc6c7ccccc7c3c4c56)cc3ccccc23)cc1 ; c1ccc(-c2cccc3cc(-c4ccc5ccc6cc(-c7cc8cccc9oc%10cccc7c%10c89)cc7c8ccccc8c4c5c67)ccc23)cc1]

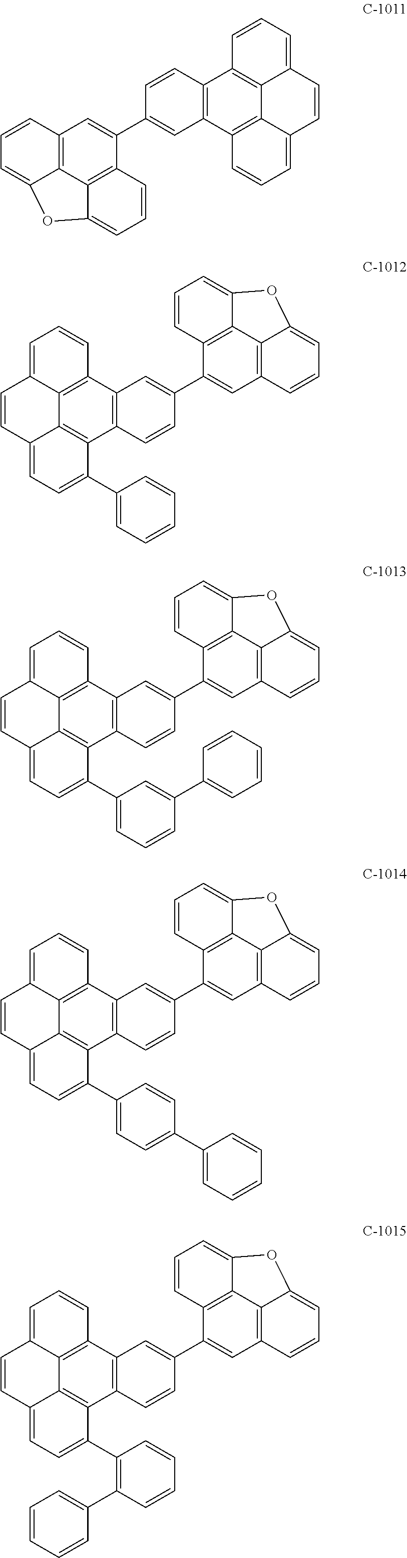 [SMILES: c1cc2cc(-c3ccc4c(c3)c3cccc5ccc6cccc4c6c53)c3cccc4oc(c1)c2c43 ; c1ccc(-c2ccc3ccc4cccc5c6cc(-c7cc8cccc9oc%10cccc7c%10c89)ccc6c2c3c45)cc1 ; c1ccc(-c2cccc(-c3ccc4ccc5cccc6c7cc(-c8cc9cccc%10oc%11cccc8c%11c9%10)ccc7c3c4c56)c2)cc1 ; c1ccc(-c2ccc(-c3ccc4ccc5cccc6c7cc(-c8cc9cccc%10oc%11cccc8c%11c9%10)ccc7c3c4c56)cc2)cc1 ; c1ccc(-c2ccccc2-c2ccc3ccc4cccc5c6cc(-c7cc8cccc9oc%10cccc7c%10c89)ccc6c2c3c45)cc1]

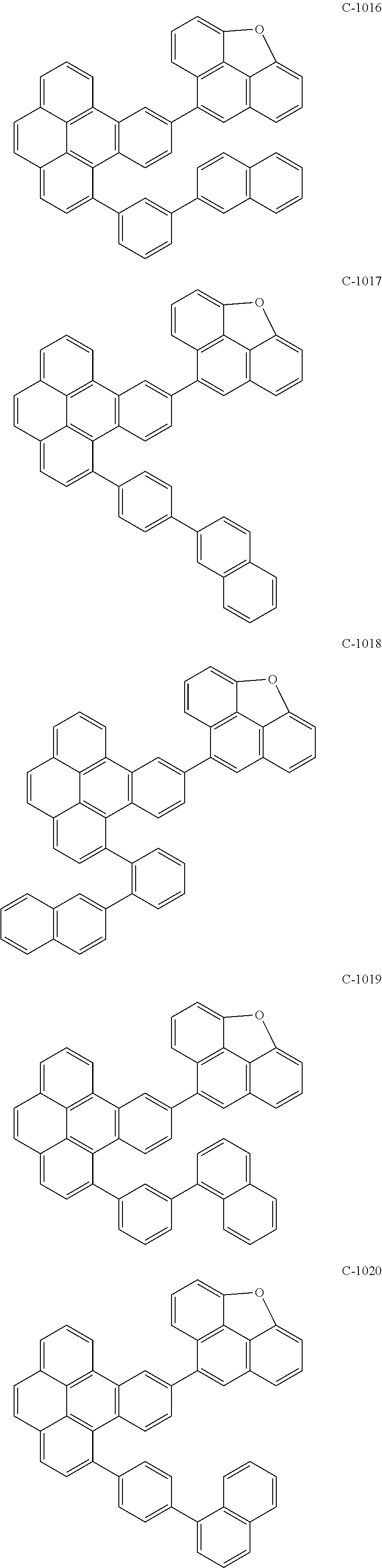 [SMILES: c1cc(-c2ccc3ccccc3c2)cc(-c2ccc3ccc4cccc5c6cc(-c7cc8cccc9oc%10cccc7c%10c89)ccc6c2c3c45)c1 ; c1ccc2cc(-c3ccc(-c4ccc5ccc6cccc7c8cc(-c9cc%10cccc%11oc%12cccc9c%12c%10%11)ccc8c4c5c67)cc3)ccc2c1 ; c1ccc(-c2ccc3ccc4cccc5c6cc(-c7cc8cccc9oc%10cccc7c%10c89)ccc6c2c3c45)c(-c2ccc3ccccc3c2)c1 ; c1cc(-c2cccc3ccccc23)cc(-c2ccc3ccc4cccc5c6cc(-c7cc8cccc9oc%10cccc7c%10c89)ccc6c2c3c45)c1 ; c1ccc2c(-c3ccc(-c4ccc5ccc6cccc7c8cc(-c9cc%10cccc%11oc%12cccc9c%12c%10%11)ccc8c4c5c67)cc3)cccc2c1]

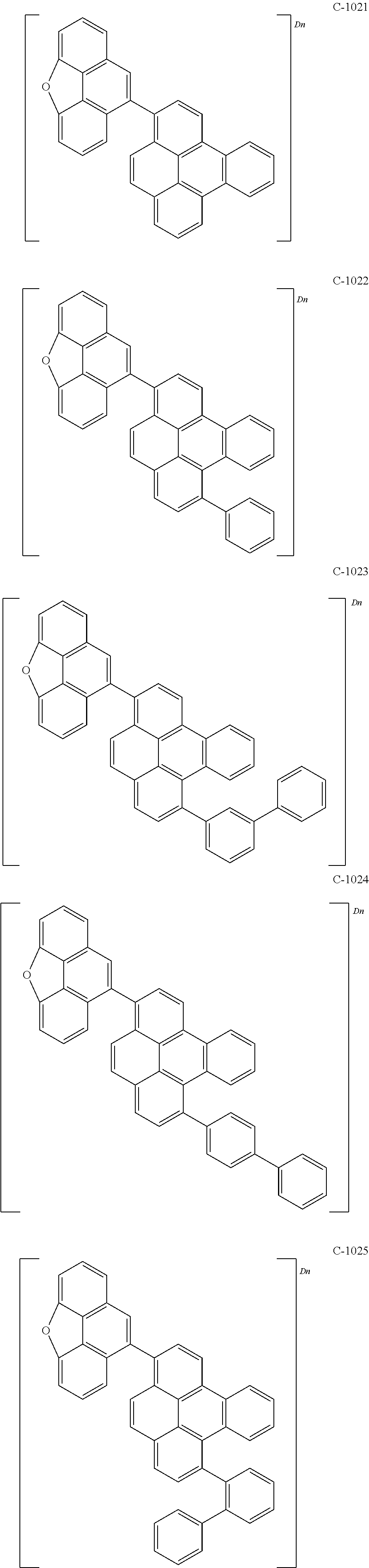

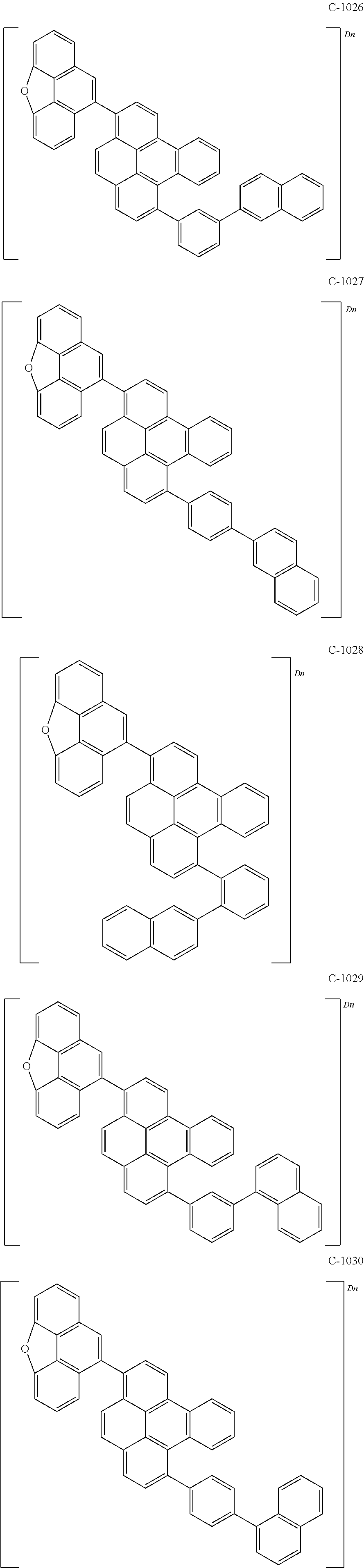

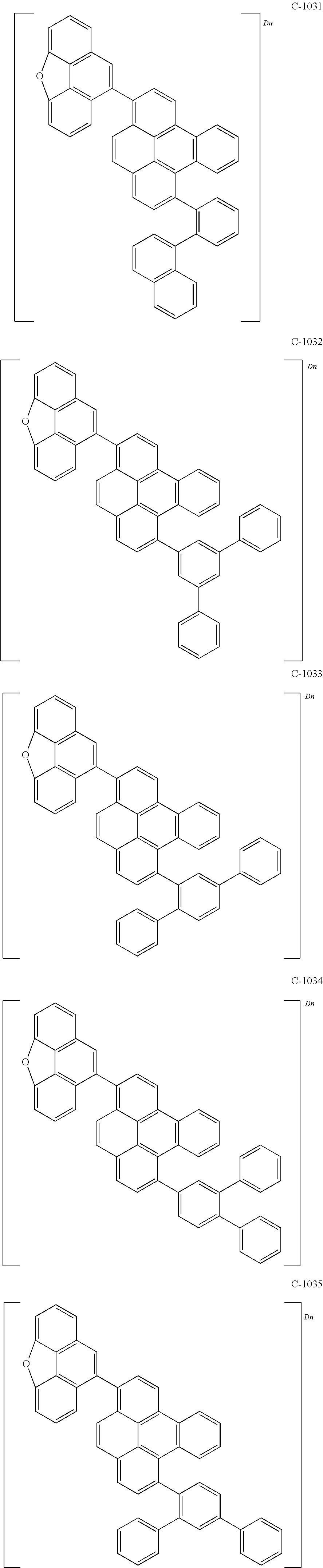

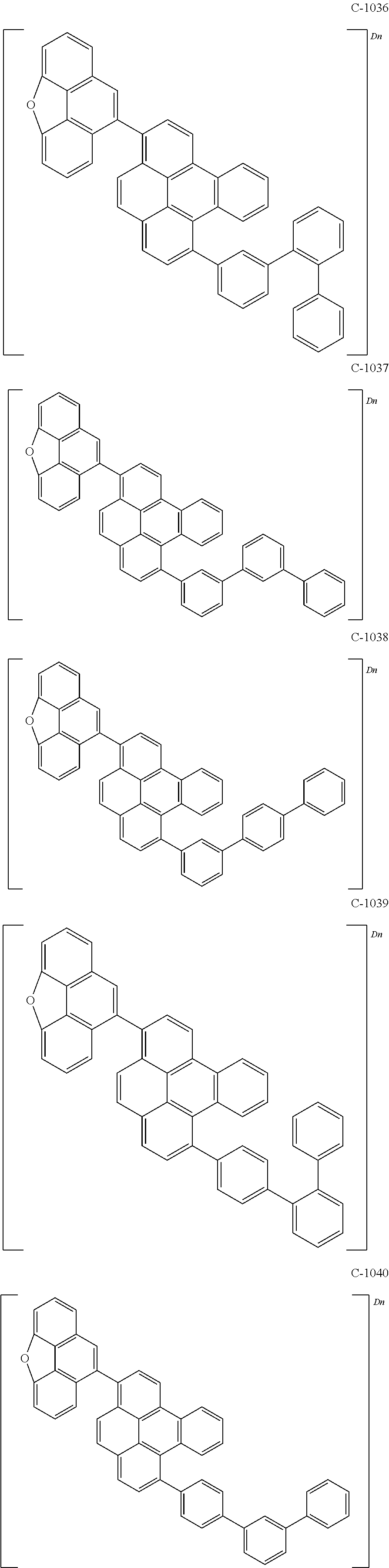

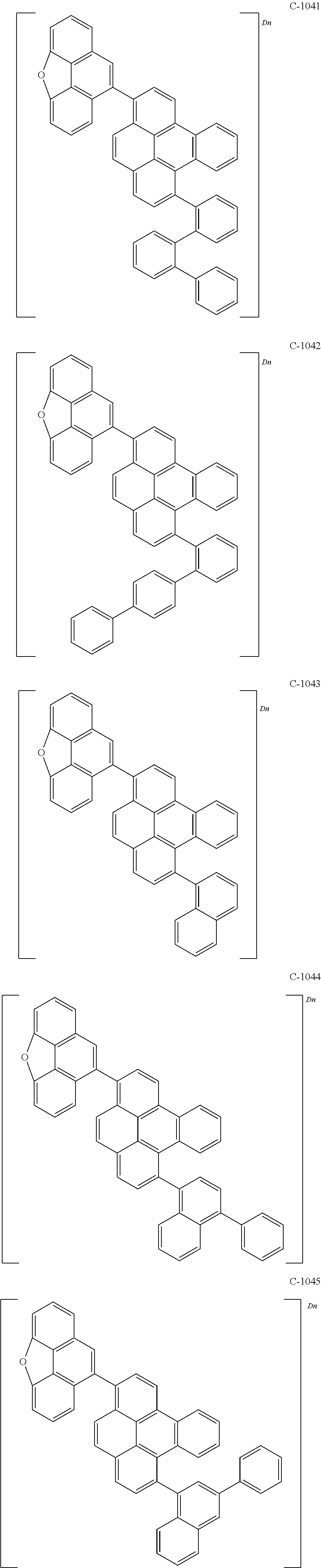

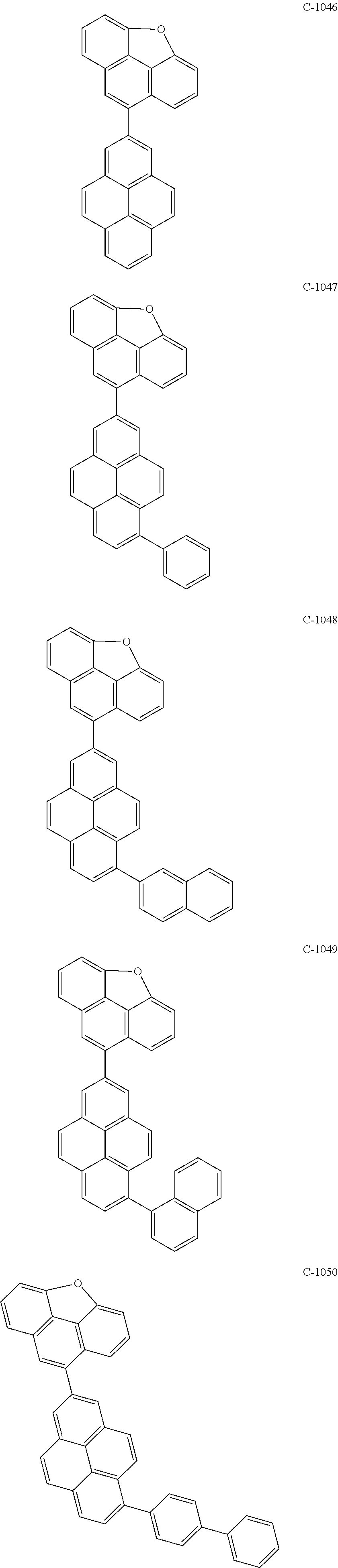 [SMILES: c1cc2ccc3cc(-c4cc5cccc6oc7cccc4c7c56)cc4ccc(c1)c2c34 ; c1ccc(-c2ccc3ccc4cc(-c5cc6cccc7oc8cccc5c8c67)cc5ccc2c3c45)cc1 ; c1ccc2cc(-c3ccc4ccc5cc(-c6cc7cccc8oc9cccc6c9c78)cc6ccc3c4c56)ccc2c1 ; c1ccc2c(-c3ccc4ccc5cc(-c6cc7cccc8oc9cccc6c9c78)cc6ccc3c4c56)cccc2c1 ; c1ccc(-c2ccc(-c3ccc4ccc5cc(-c6cc7cccc8oc9cccc6c9c78)cc6ccc3c4c56)cc2)cc1]

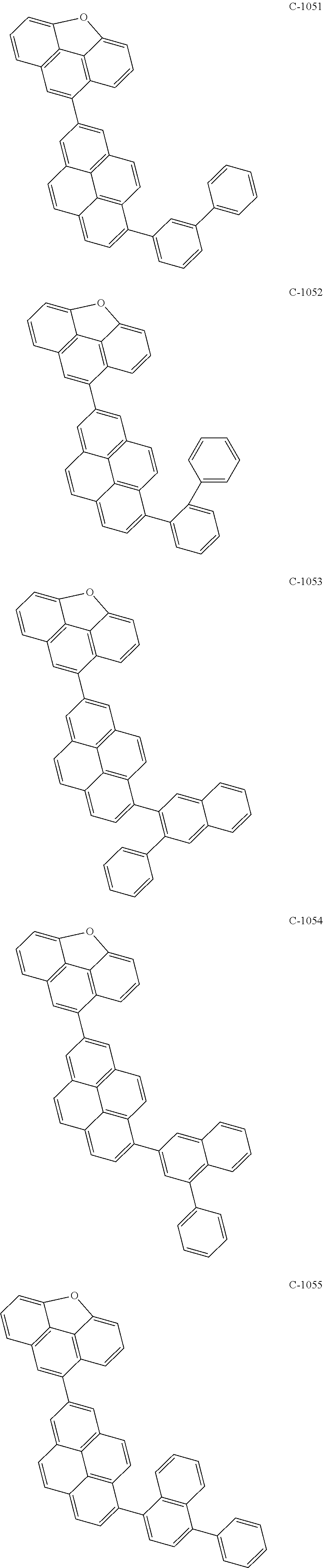 [SMILES: c1ccc(-c2cccc(-c3ccc4ccc5cc(-c6cc7cccc8oc9cccc6c9c78)cc6ccc3c4c56)c2)cc1 ; c1ccc(-c2ccccc2-c2ccc3ccc4cc(-c5cc6cccc7oc8cccc5c8c67)cc5ccc2c3c45)cc1 ; c1ccc(-c2cc3ccccc3cc2-c2ccc3ccc4cc(-c5cc6cccc7oc8cccc5c8c67)cc5ccc2c3c45)cc1 ; c1ccc(-c2cc(-c3ccc4ccc5cc(-c6cc7cccc8oc9cccc6c9c78)cc6ccc3c4c56)cc3ccccc23)cc1 ; c1ccc(-c2ccc(-c3ccc4ccc5cc(-c6cc7cccc8oc9cccc6c9c78)cc6ccc3c4c56)c3ccccc23)cc1]

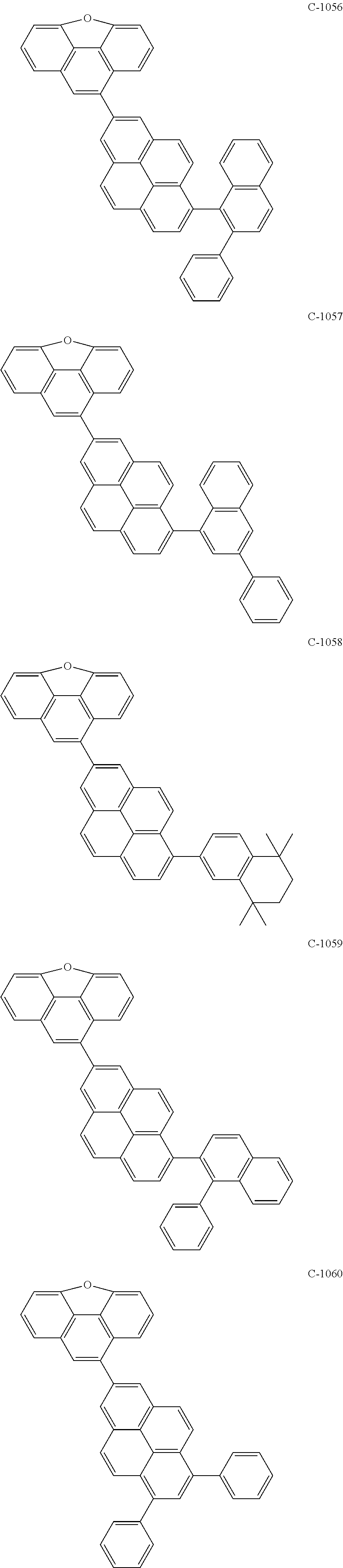 [SMILES: c1ccc(-c2ccc3ccccc3c2-c2ccc3ccc4cc(-c5cc6cccc7oc8cccc5c8c67)cc5ccc2c3c45)cc1 ; c1ccc(-c2cc(-c3ccc4ccc5cc(-c6cc7cccc8oc9cccc6c9c78)cc6ccc3c4c56)c3ccccc3c2)cc1 ; CC1(C)CCC(C)(C)c2cc(-c3ccc4ccc5cc(-c6cc7cccc8oc9cccc6c9c78)cc6ccc3c4c56)ccc21 ; c1ccc(-c2c(-c3ccc4ccc5cc(-c6cc7cccc8oc9cccc6c9c78)cc6ccc3c4c56)ccc3ccccc23)cc1 ; c1ccc(-c2cc(-c3ccccc3)c3ccc4cc(-c5cc6cccc7oc8cccc5c8c67)cc5ccc2c3c54)cc1]

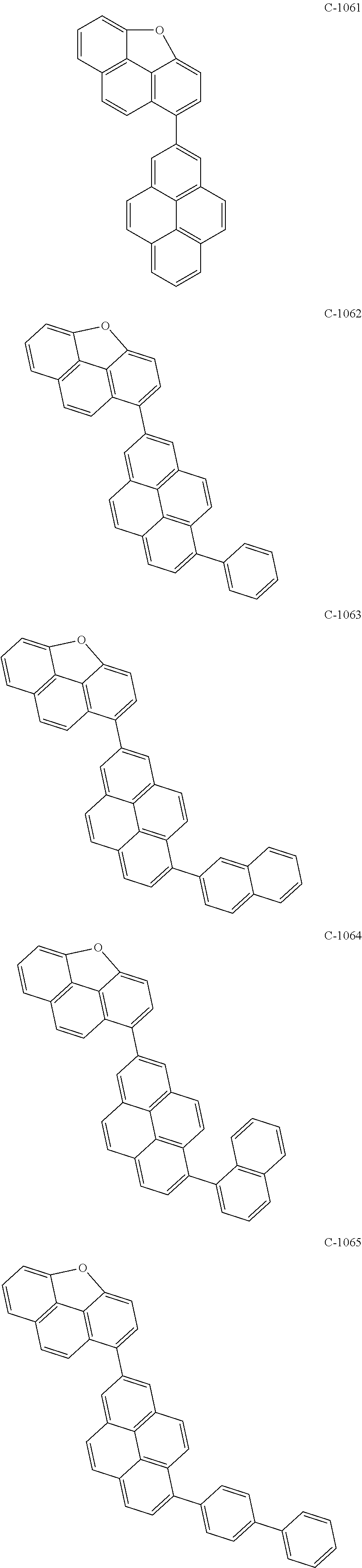 [SMILES: c1cc2ccc3cc(-c4ccc5oc6cccc7ccc4c5c76)cc4ccc(c1)c2c34 ; c1ccc(-c2ccc3ccc4cc(-c5ccc6oc7cccc8ccc5c6c87)cc5ccc2c3c45)cc1 ; c1ccc2cc(-c3ccc4ccc5cc(-c6ccc7oc8cccc9ccc6c7c98)cc6ccc3c4c56)ccc2c1 ; c1ccc2c(-c3ccc4ccc5cc(-c6ccc7oc8cccc9ccc6c7c98)cc6ccc3c4c56)cccc2c1 ; c1ccc(-c2ccc(-c3ccc4ccc5cc(-c6ccc7oc8cccc9ccc6c7c98)cc6ccc3c4c56)cc2)cc1]

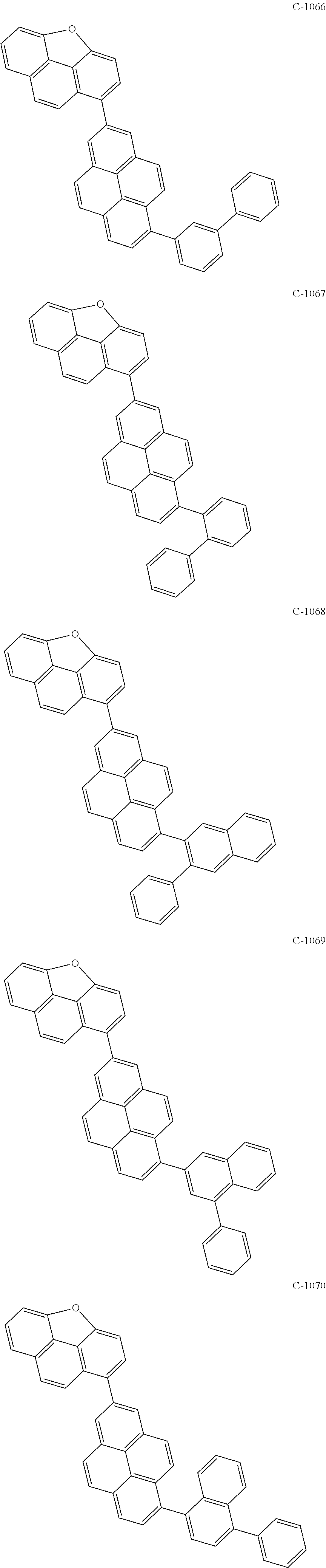 [SMILES: c1ccc(-c2cccc(-c3ccc4ccc5cc(-c6ccc7oc8cccc9ccc6c7c98)cc6ccc3c4c56)c2)cc1 ; c1ccc(-c2ccccc2-c2ccc3ccc4cc(-c5ccc6oc7cccc8ccc5c6c87)cc5ccc2c3c45)cc1 ; c1ccc(-c2cc3ccccc3cc2-c2ccc3ccc4cc(-c5ccc6oc7cccc8ccc5c6c87)cc5ccc2c3c45)cc1 ; c1ccc(-c2cc(-c3ccc4ccc5cc(-c6ccc7oc8cccc9ccc6c7c98)cc6ccc3c4c56)cc3ccccc23)cc1 ; c1ccc(-c2ccc(-c3ccc4ccc5cc(-c6ccc7oc8cccc9ccc6c7c98)cc6ccc3c4c56)c3ccccc23)cc1]

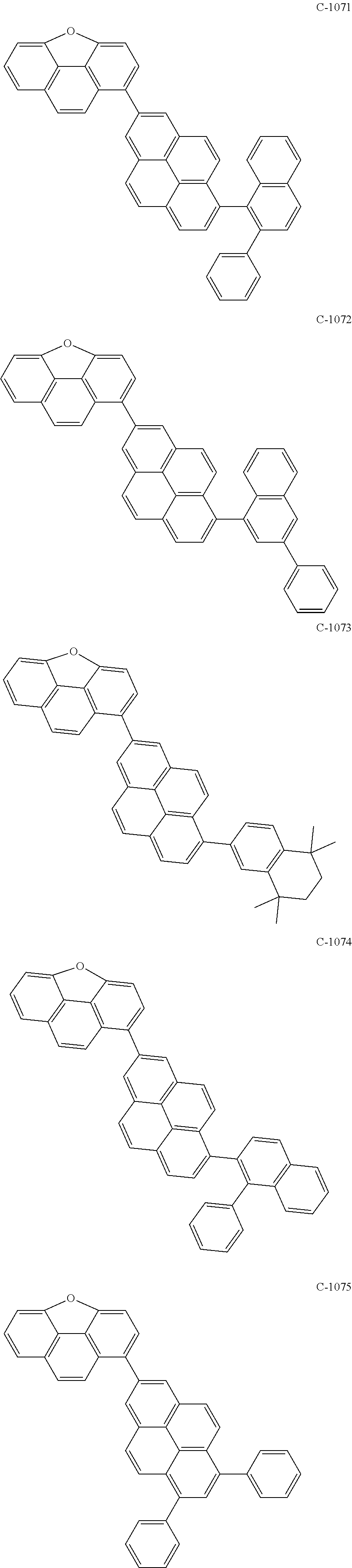 [SMILES: c1ccc(-c2ccc3ccccc3c2-c2ccc3ccc4cc(-c5ccc6oc7cccc8ccc5c6c87)cc5ccc2c3c45)cc1 ; c1ccc(-c2cc(-c3ccc4ccc5cc(-c6ccc7oc8cccc9ccc6c7c98)cc6ccc3c4c56)c3ccccc3c2)cc1 ; CC1(C)CCC(C)(C)c2cc(-c3ccc4ccc5cc(-c6ccc7oc8cccc9ccc6c7c98)cc6ccc3c4c56)ccc21 ; c1ccc(-c2c(-c3ccc4ccc5cc(-c6ccc7oc8cccc9ccc6c7c98)cc6ccc3c4c56)ccc3ccccc23)cc1 ; c1ccc(-c2cc(-c3ccccc3)c3ccc4cc(-c5ccc6oc7cccc8ccc5c6c87)cc5ccc2c3c54)cc1]

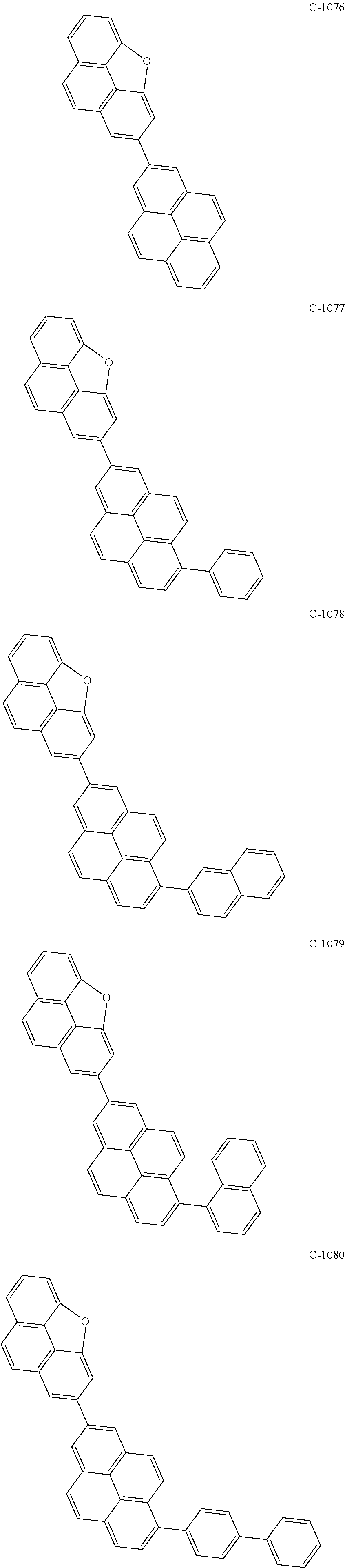 [SMILES: c1cc2ccc3cc(-c4cc5ccc6cccc7oc(c4)c5c67)cc4ccc(c1)c2c34 ; c1ccc(-c2ccc3ccc4cc(-c5cc6ccc7cccc8oc(c5)c6c78)cc5ccc2c3c45)cc1 ; c1ccc2cc(-c3ccc4ccc5cc(-c6cc7ccc8cccc9oc(c6)c7c89)cc6ccc3c4c56)ccc2c1 ; c1ccc2c(-c3ccc4ccc5cc(-c6cc7ccc8cccc9oc(c6)c7c89)cc6ccc3c4c56)cccc2c1 ; c1ccc(-c2ccc(-c3ccc4ccc5cc(-c6cc7ccc8cccc9oc(c6)c7c89)cc6ccc3c4c56)cc2)cc1]

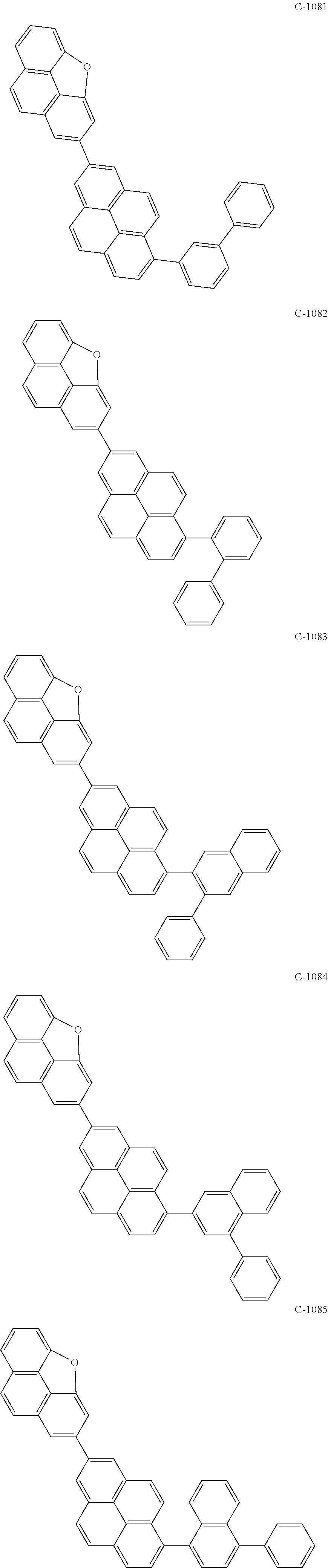 [SMILES: c1ccc(-c2cccc(-c3ccc4ccc5cc(-c6cc7ccc8cccc9oc(c6)c7c89)cc6ccc3c4c56)c2)cc1 ; c1ccc(-c2ccccc2-c2ccc3ccc4cc(-c5cc6ccc7cccc8oc(c5)c6c78)cc5ccc2c3c45)cc1 ; c1ccc(-c2cc3ccccc3cc2-c2ccc3ccc4cc(-c5cc6ccc7cccc8oc(c5)c6c78)cc5ccc2c3c45)cc1 ; c1ccc(-c2cc(-c3ccc4ccc5cc(-c6cc7ccc8cccc9oc(c6)c7c89)cc6ccc3c4c56)cc3ccccc23)cc1 ; c1ccc(-c2ccc(-c3ccc4ccc5cc(-c6cc7ccc8cccc9oc(c6)c7c89)cc6ccc3c4c56)c3ccccc23)cc1]

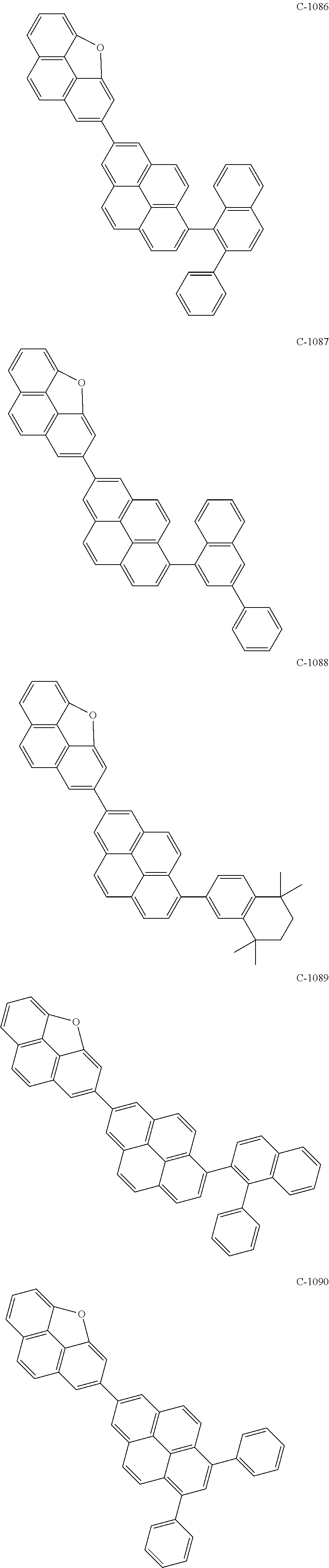 [SMILES: c1ccc(-c2ccc3ccccc3c2-c2ccc3ccc4cc(-c5cc6ccc7cccc8oc(c5)c6c78)cc5ccc2c3c45)cc1 ; c1ccc(-c2cc(-c3ccc4ccc5cc(-c6cc7ccc8cccc9oc(c6)c7c89)cc6ccc3c4c56)c3ccccc3c2)cc1 ; CC1(C)CCC(C)(C)c2cc(-c3ccc4ccc5cc(-c6cc7ccc8cccc9oc(c6)c7c89)cc6ccc3c4c56)ccc21 ; c1ccc(-c2c(-c3ccc4ccc5cc(-c6cc7ccc8cccc9oc(c6)c7c89)cc6ccc3c4c56)ccc3ccccc23)cc1 ; c1ccc(-c2cc(-c3ccccc3)c3ccc4cc(-c5cc6ccc7cccc8oc(c5)c6c78)cc5ccc2c3c54)cc1]

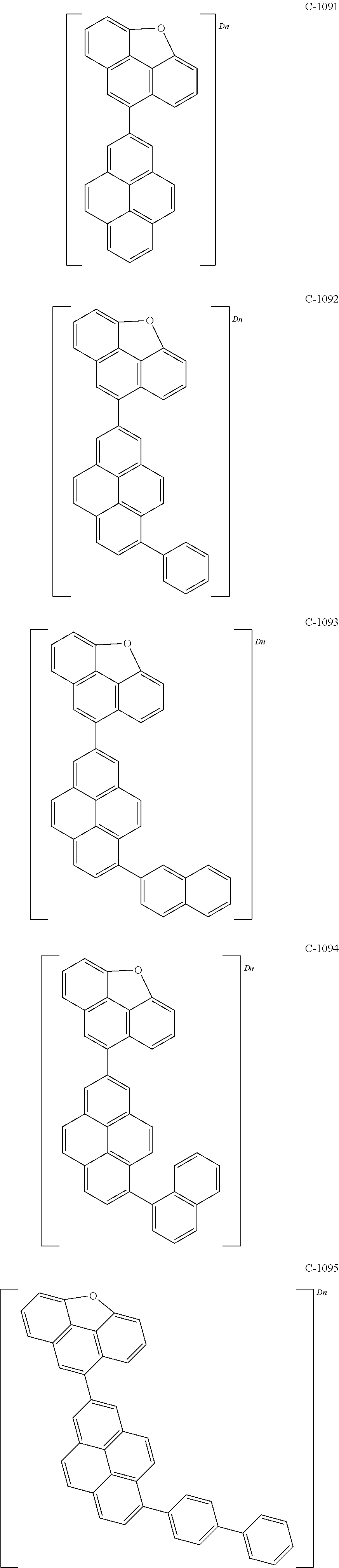

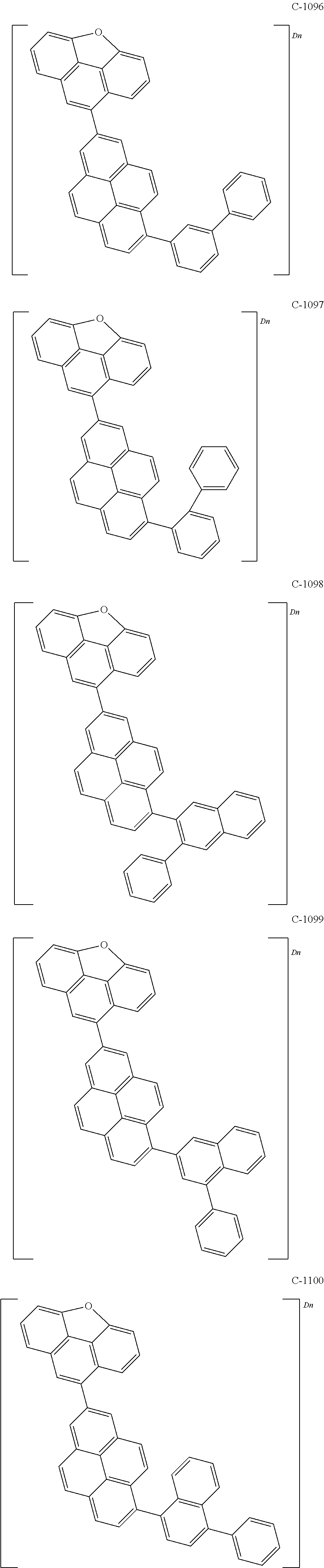

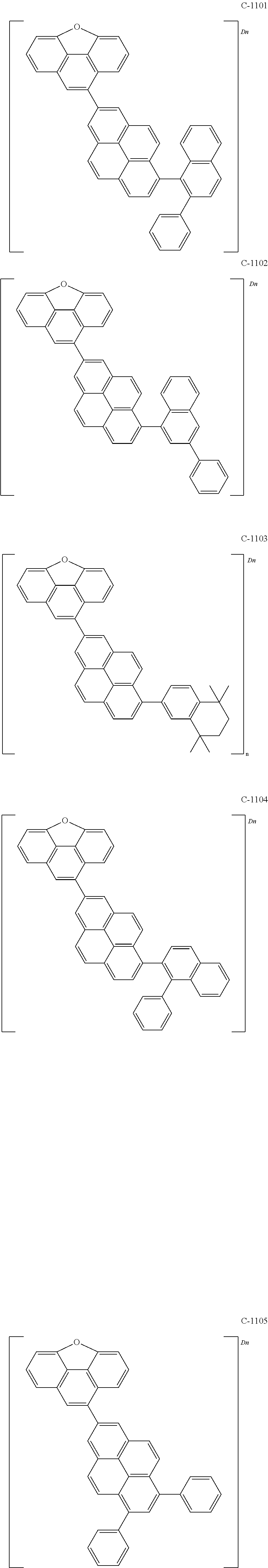

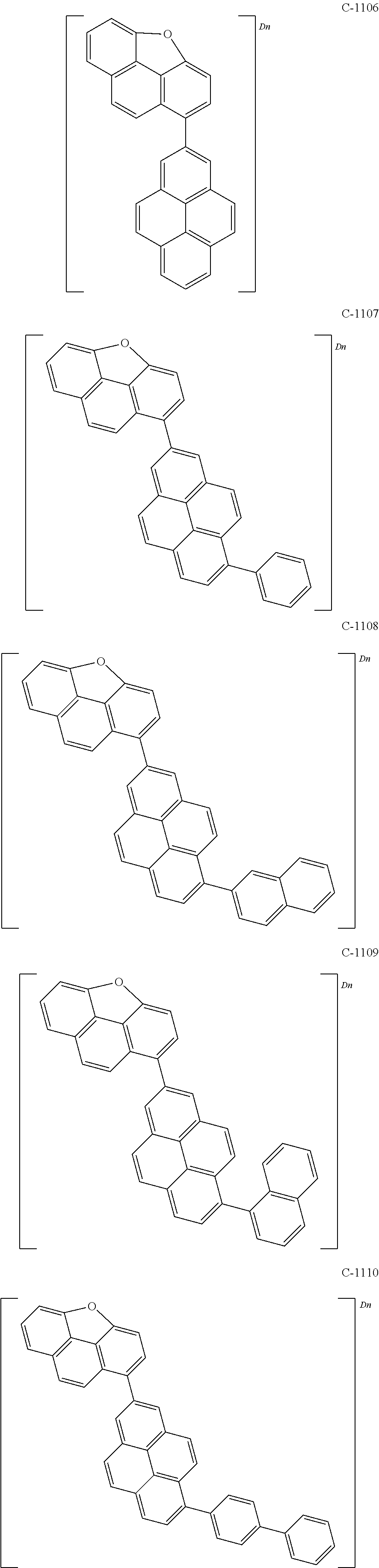

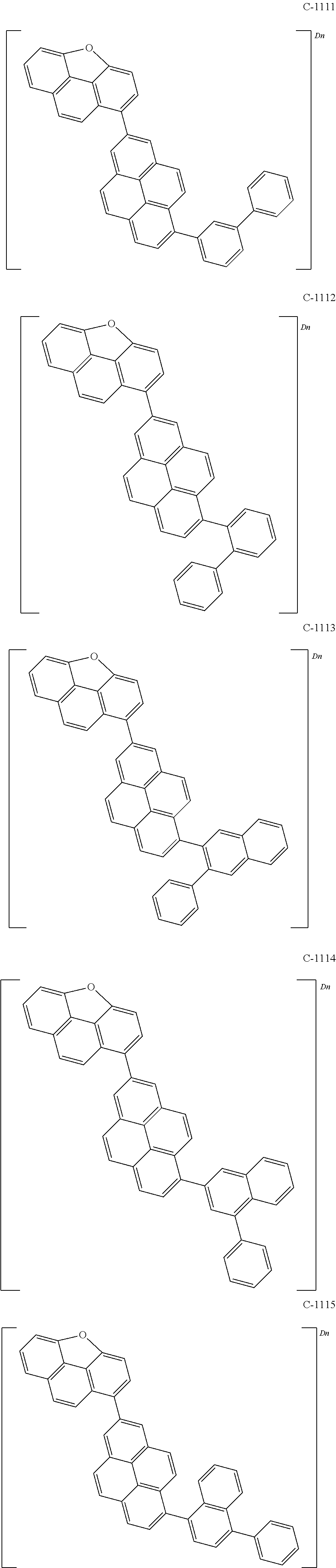

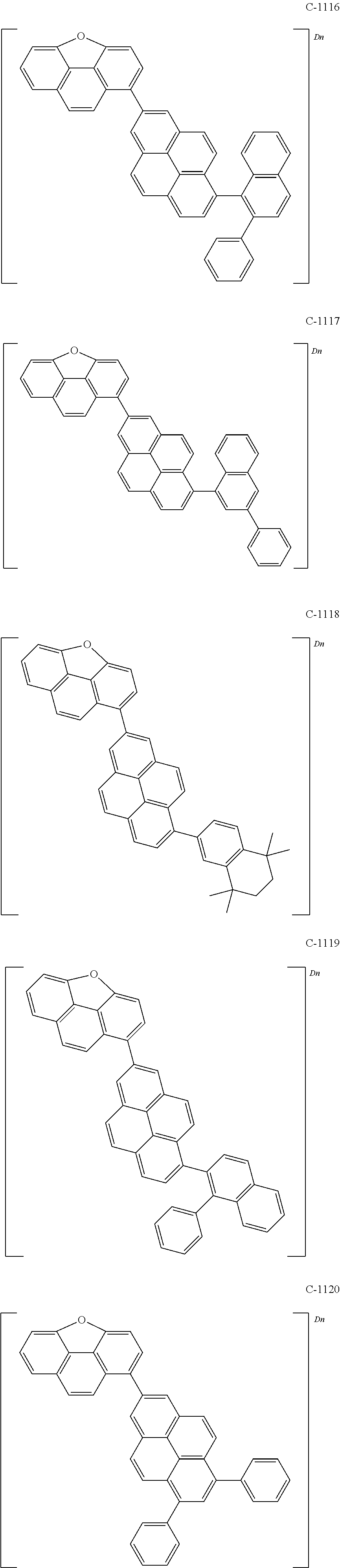

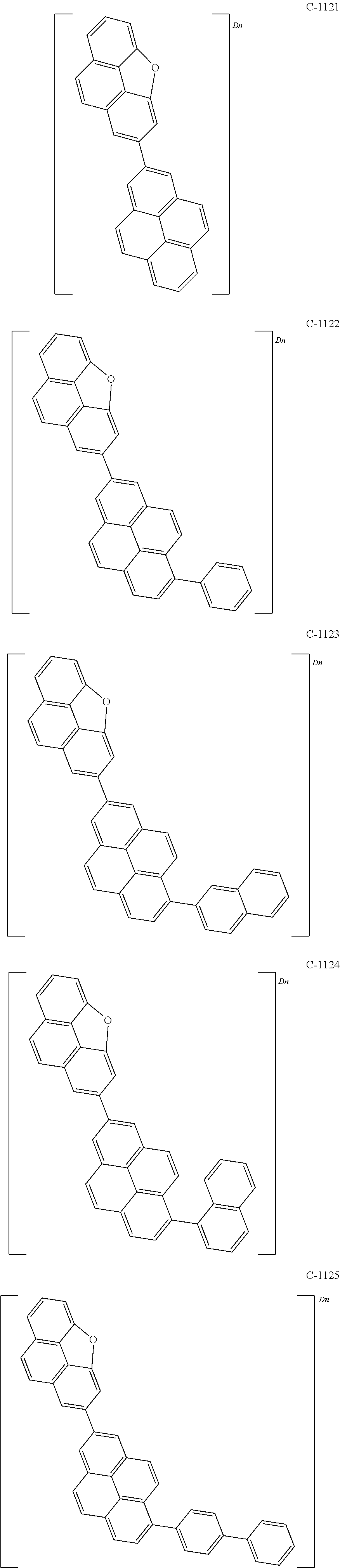

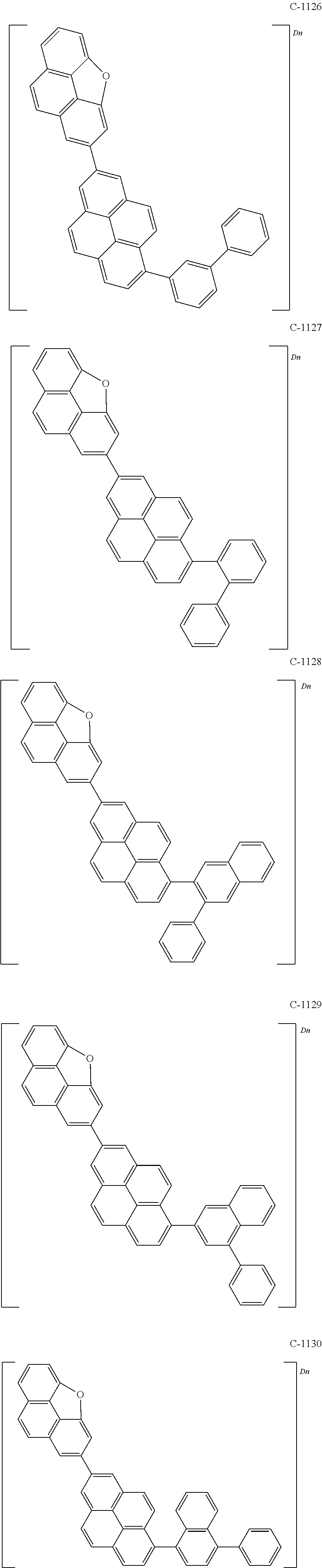

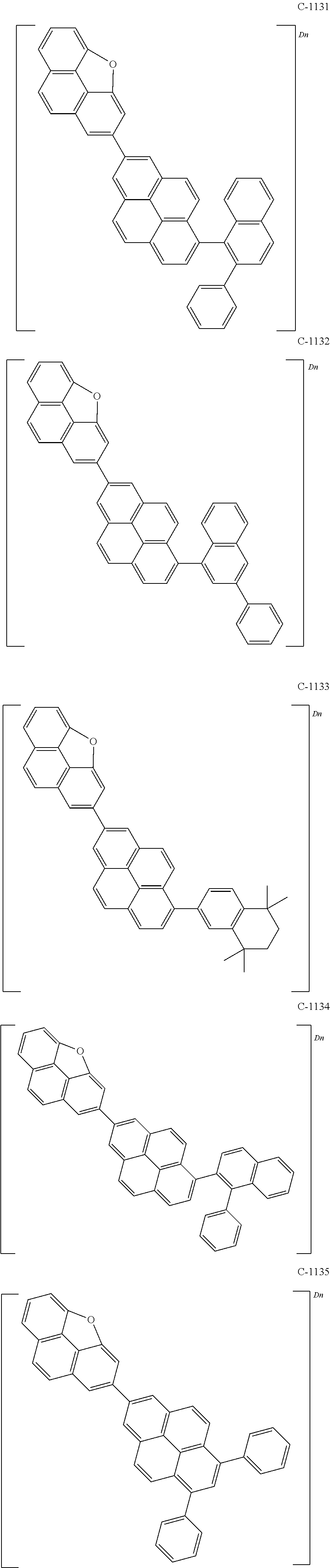

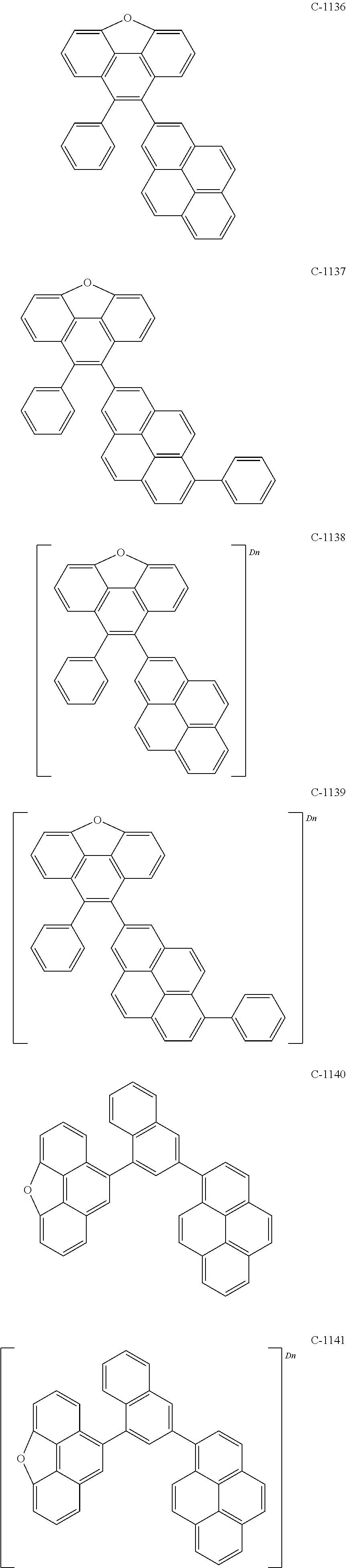

In the above compounds, Dₙ indicates that n hydrogen atoms are replaced with deuterium, and n ranges from 1 to the total number of hydrogen atoms in the compound.

An organic electroluminescent compound according to another embodiment of the present disclosure is represented by the following Formula 2.

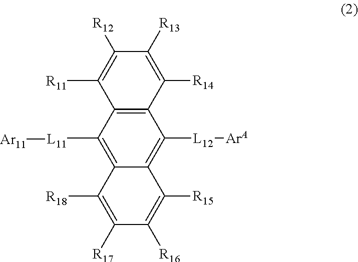

In Formula 2,
R₁₁ to R₁₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of a (C₃-C₃₀) aliphatic ring and a (C₆-C₃₀) aromatic ring, or -L₁₃-N(Ar₁₃)(Ar₁₄);
L₁₁ and L₁₂ each independently represent a single bond, a substituted or unsubstituted (C₆-C₃₀)arylene, or a substituted or unsubstituted (3- to 30-membered)heteroarylene;
Ar₁₁ represents a substituted or unsubstituted (C₆-C₃₀)aryl or a substituted or unsubstituted (3- to 30-membered)heteroaryl; and
Ar^{A} represents a substituted or unsubstituted (C₆-C₃₀)aryl, a substituted or unsubstituted (3- to 30-membered)heteroaryl, or the following Formula A-1:

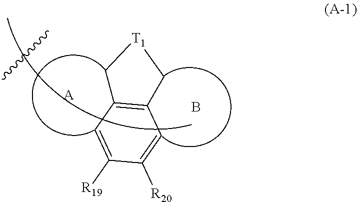

wherein in Formula A-1,
T₁ represents —O—, —S—, —CRₐR_{b}, or —NR_{c};
ring A and ring B each independently represent a substituted or unsubstituted (C₆-C₃₀) arene ring, or a substituted or unsubstituted (3- to 30-membered)heteroarene ring;
R₁₉ and R₂₀ each independently represent a site linked to L₁₂, or hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of a (C₃-C₃₀) aliphatic ring and a (C₆-C₃₀) aromatic ring, or -L₁₃-N(Ar₁₃)(Ar₁₄);
Rₐ and R_{b} each independently represent a substituted or unsubstituted (C₁-C₃₀)alkyl, or a substituted or unsubstituted (C₆-C₃₀)aryl; or may be linked to each other to form a ring;
R_{c} represents a substituted or unsubstituted (C₁-C₃₀)alkyl or a substituted or unsubstituted (C₆-C₃₀)aryl;
L₁₃ represents a single bond, a substituted or unsubstituted (C₆-C₃₀)arylene, or a substituted or unsubstituted (3- to 30-membered)heteroarylene; and
Ar₁₃ and Ar₁₄ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted fused ring of a (C₃-C₃₀) aliphatic ring and a (C₆-C₃₀) aromatic ring, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl.

In one embodiment, R₁₁ to R₁₈ each independently may be hydrogen or deuterium.

In one embodiment, R₁₉ and R₂₀ each independently may be a site linked to L₁₂, hydrogen, or deuterium.

In one embodiment, L₁₁ and L₁₂ each independently may be a single bond or a substituted or unsubstituted (C₆-C₃₀)arylene, preferably a single bond or (C₆-C₂₅)arylene unsubstituted or substituted with (C₆-C₃₀)aryl, more preferably a single bond or (C₆-C₁₈)arylene unsubstituted or substituted with (C₆-C₃₀)aryl. For example, L₁₁ and L₁₂ each independently may be phenylene unsubstituted or substituted with phenyl, naphthylene unsubstituted or substituted with phenyl, a substituted or unsubstituted phenanthrenylene, wherein the substituents may be further substituted with at least one deuterium.

In one embodiment, Ar₁₁ and Ar^{A} each independently may be a substituted or unsubstituted (C₆-C₃₀)aryl or a substituted or unsubstituted (5- to 30-membered)heteroaryl, preferably a substituted or unsubstituted (C₆-C₃₀)aryl or a substituted or unsubstituted (5- to 25-membered)heteroaryl, more preferably a substituted or unsubstituted (C₆-C₂₅)aryl or a substituted or unsubstituted (5- to 18-membered)heteroaryl. For example, Ar₁₁ and Ar^{A} each independently may be phenyl unsubstituted or substituted with naphthyl, naphthyl unsubstituted or substituted with phenyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted dimethylfluorenyl, a substituted or unsubstituted diphenylfluorenyl, a substituted or unsubstituted dimethylbenzofluorenyl, a substituted or unsubstituted spirobifluorenyl, a substituted or unsubstituted phenanthrenyl, a substituted or unsubstituted triphenylenyl, a substituted or unsubstituted carbazolyl, a substituted or unsubstituted dibenzofuranyl, a substituted or unsubstituted dibenzothiophenyl, a substituted or unsubstituted benzocarbazolyl, a substituted or unsubstituted naphthobenzofuranyl, or a substituted or unsubstituted naphthobenzothiophenyl, wherein the substituents can be further substituted with at least one deuterium. For example, Ar₁₁ may be phenyl unsubstituted or substituted with deuterium, biphenyl unsubstituted or substituted with deuterium, terphenyl unsubstituted or substituted with deuterium, naphthyl unsubstituted or substituted with deuterium, phenanthrenyl unsubstituted or substituted with deuterium, or a combination thereof. For example, Ar^{A} may be phenyl unsubstituted or substituted with deuterium, biphenyl unsubstituted or substituted with deuterium, terphenyl unsubstituted or substituted with deuterium, naphthyl unsubstituted or substituted with deuterium, phenanthrenyl unsubstituted or substituted with deuterium, dibenzofuranyl unsubstituted or substituted with deuterium, dibenzothiophenyl unsubstituted or substituted with deuterium, or a combination thereof.

In one embodiment, Ar^{A} may be the substituent represented by Formula A-1.

In one embodiment, T₁ in Formula A-1 may be —O—, —S—, or —CRₐR_{b}.

In one embodiment, ring A and ring B in Formula A-1 each independently may be a benzene ring or a naphthalene ring.

In one embodiment, all of ring A and ring B in Formula A-1 may be a substituted or unsubstituted benzene ring, preferably a benzene ring unsubstituted or substituted with deuterium or (C₆-C₃₀)aryl, more preferably a benzene ring unsubstituted or substituted with deuterium, phenyl, naphthyl, or biphenyl.

According to one embodiment, the organic electroluminescent compound represented by Formula 2 may be more specifically illustrated by the following compounds, but is not limited thereto:

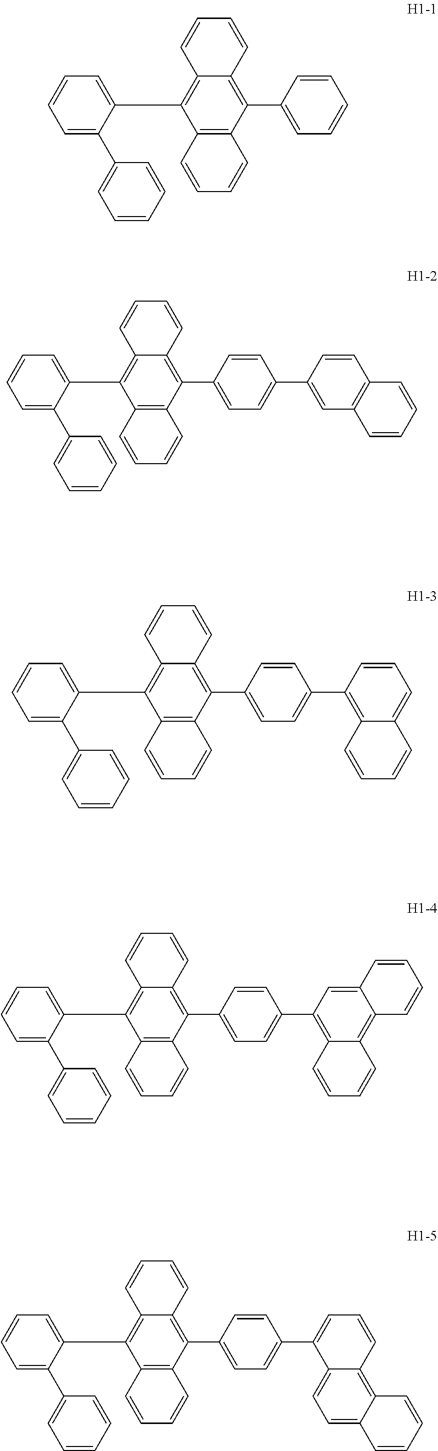

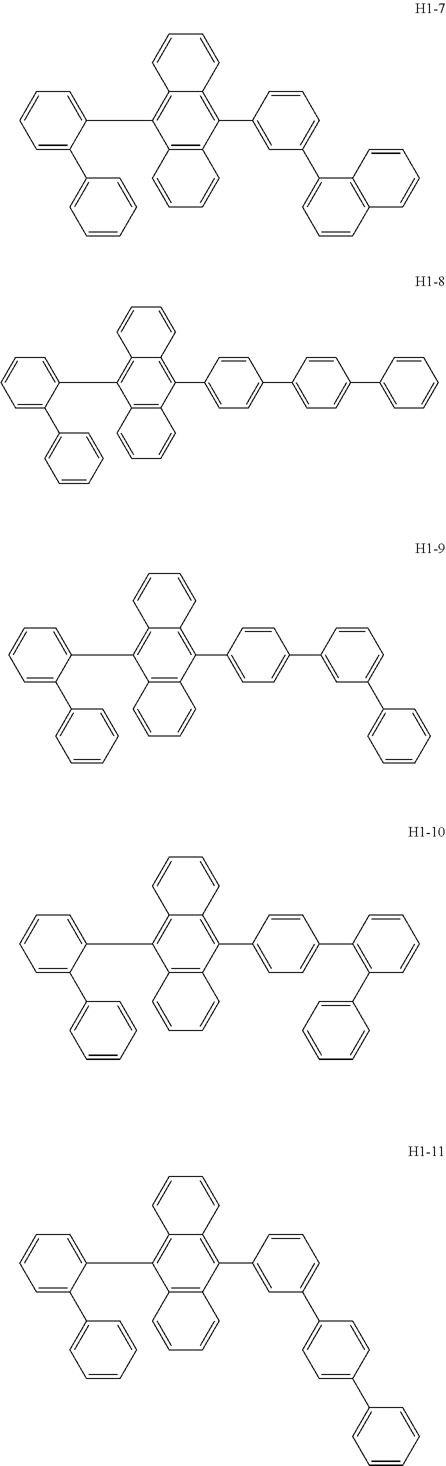

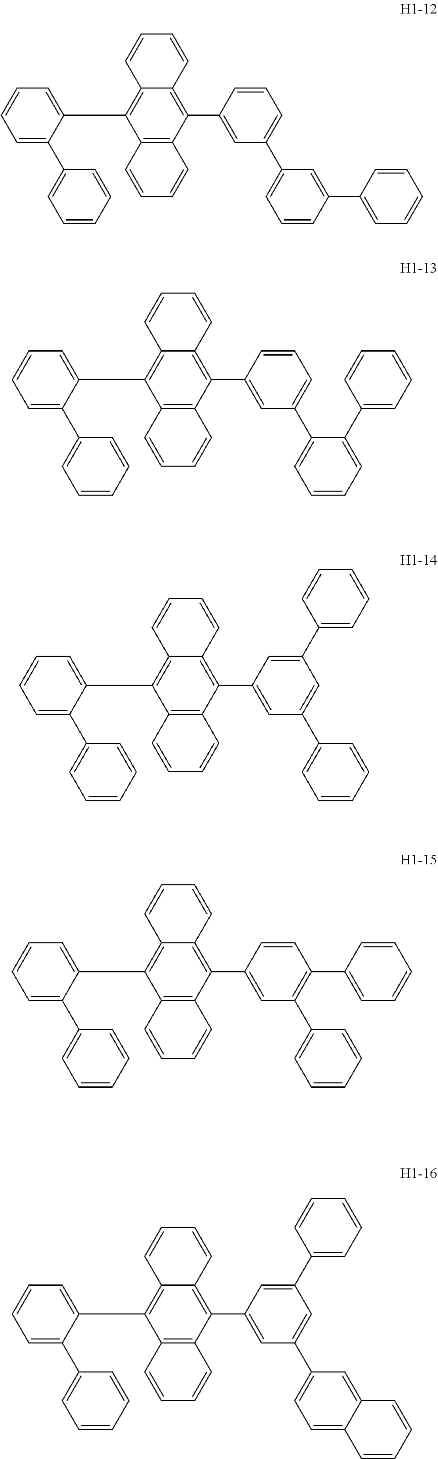

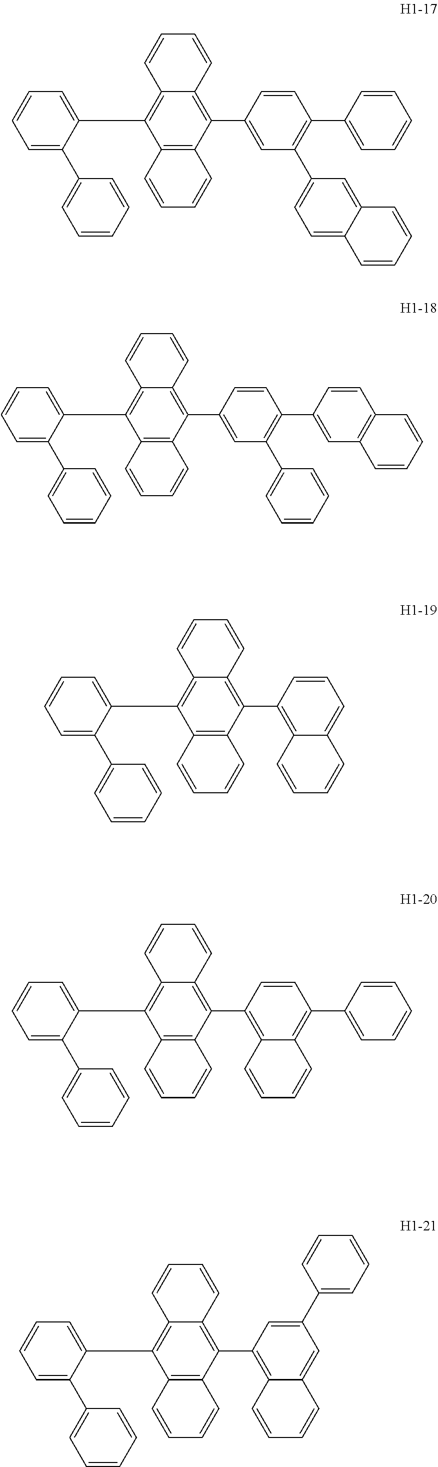

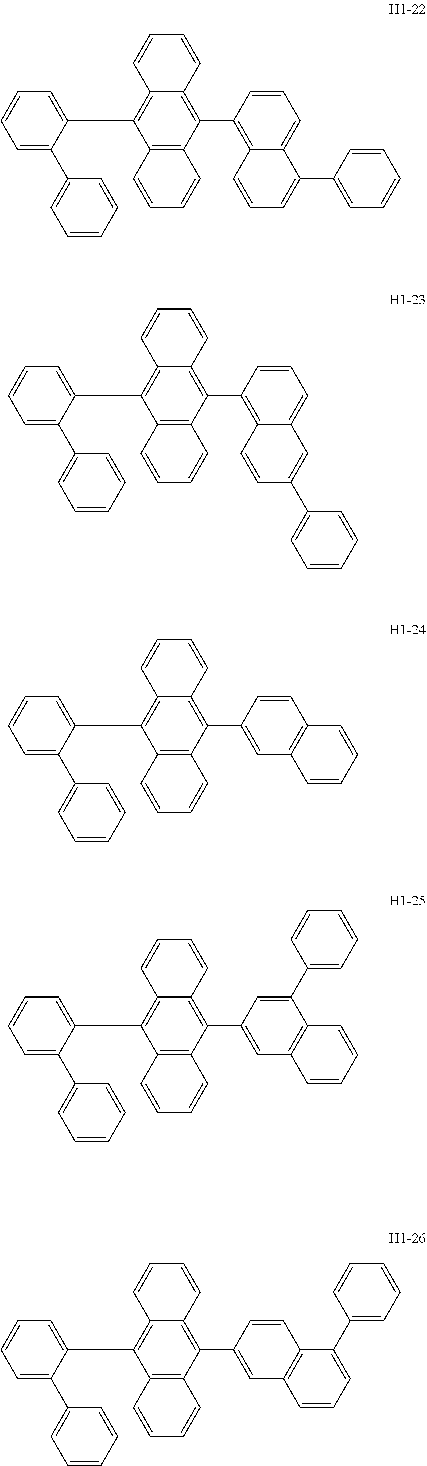

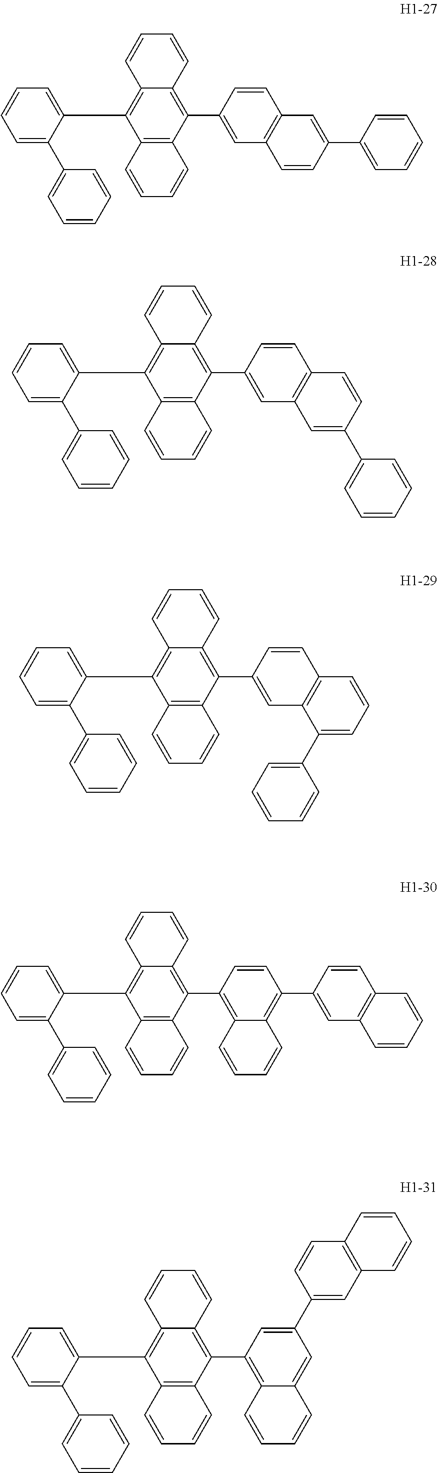

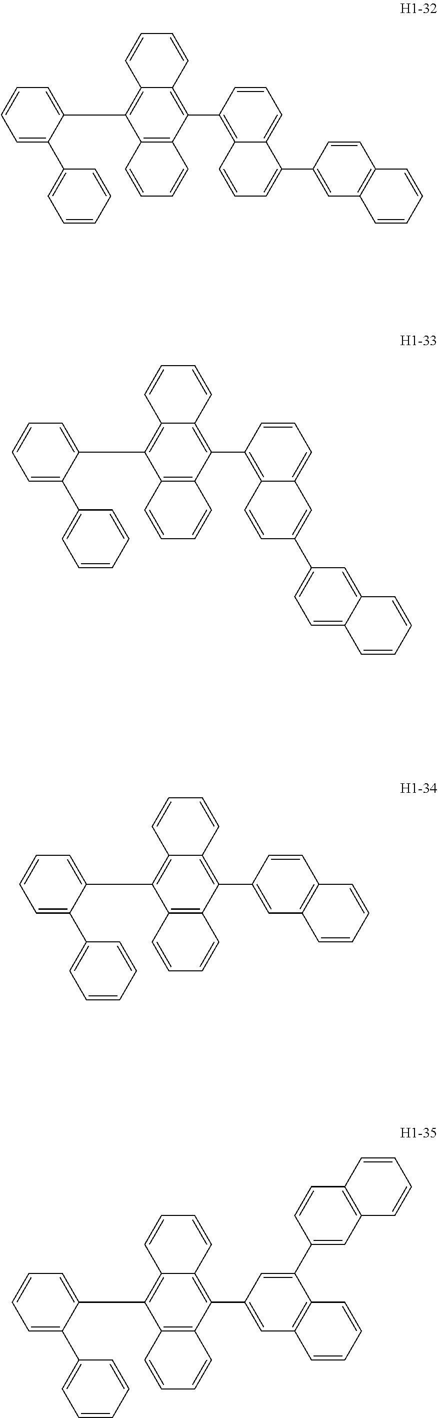

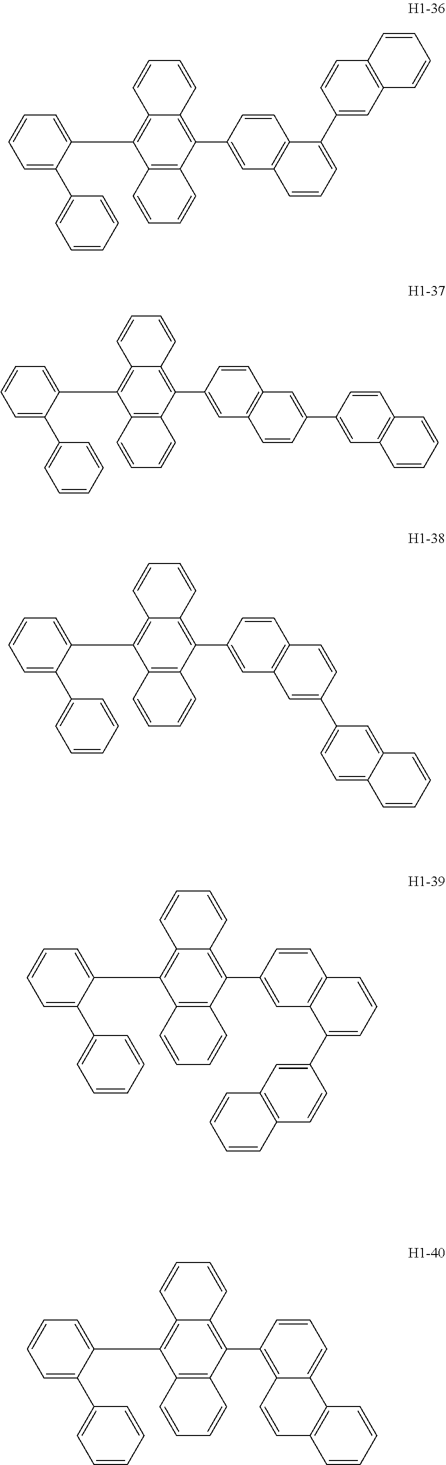

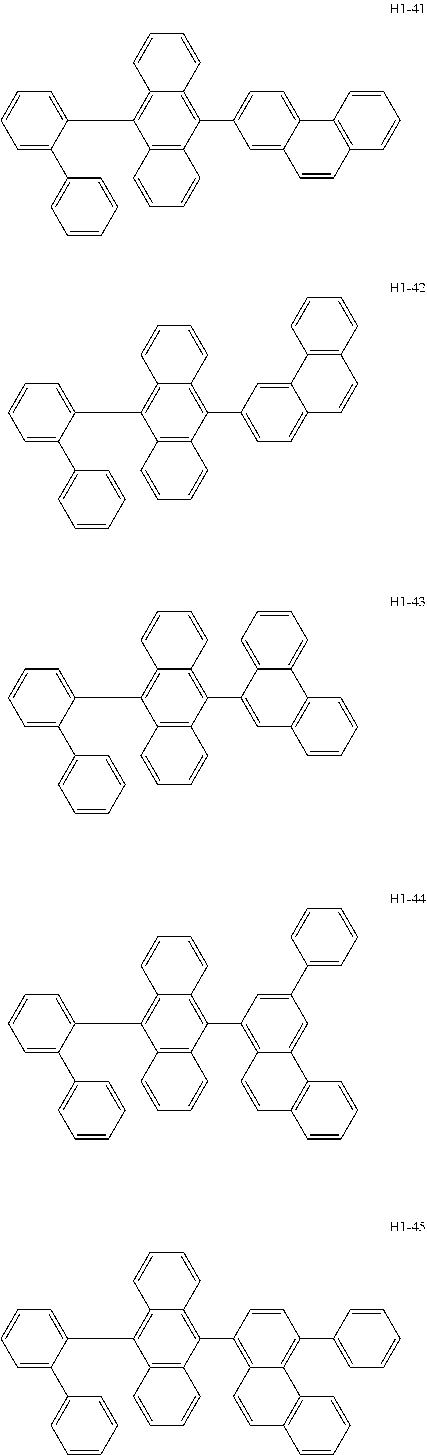

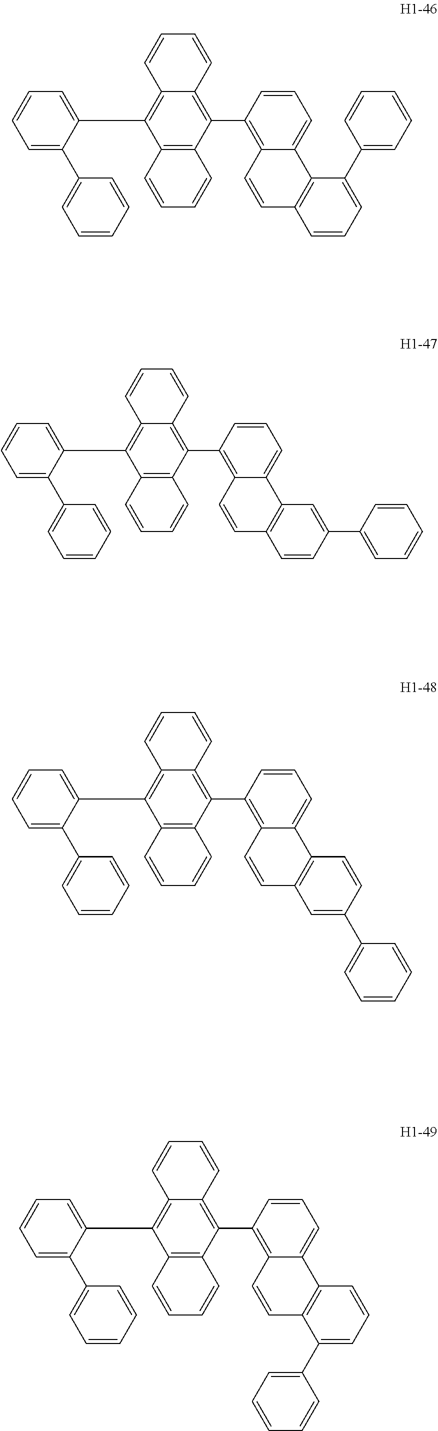

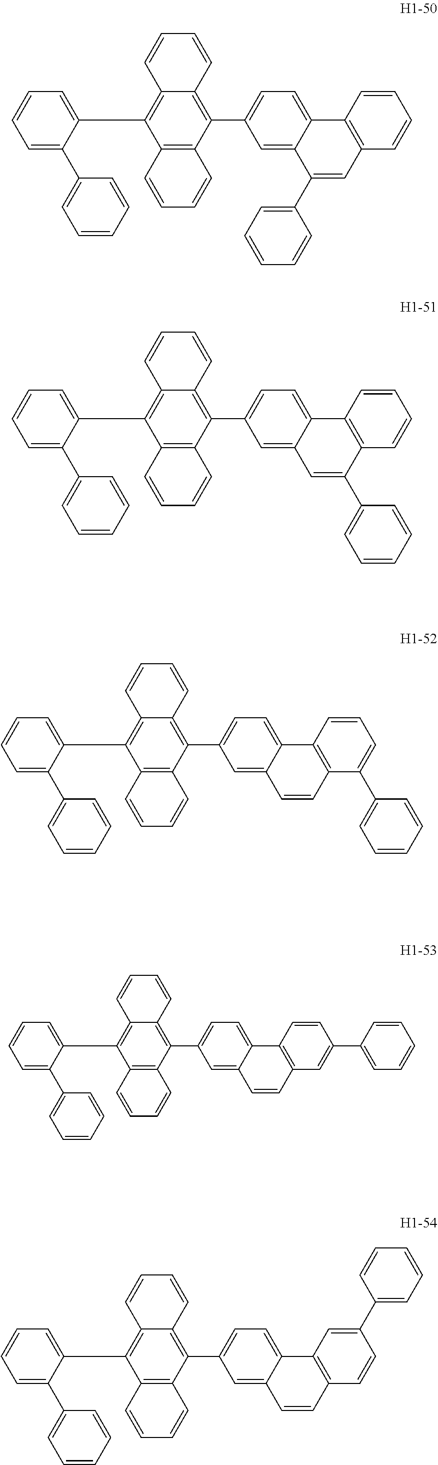

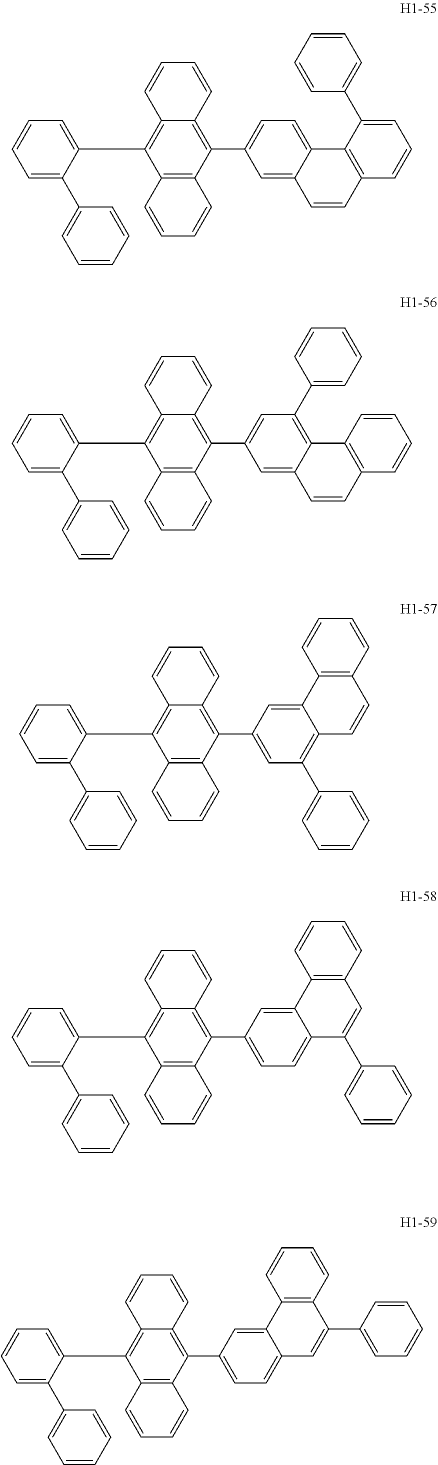

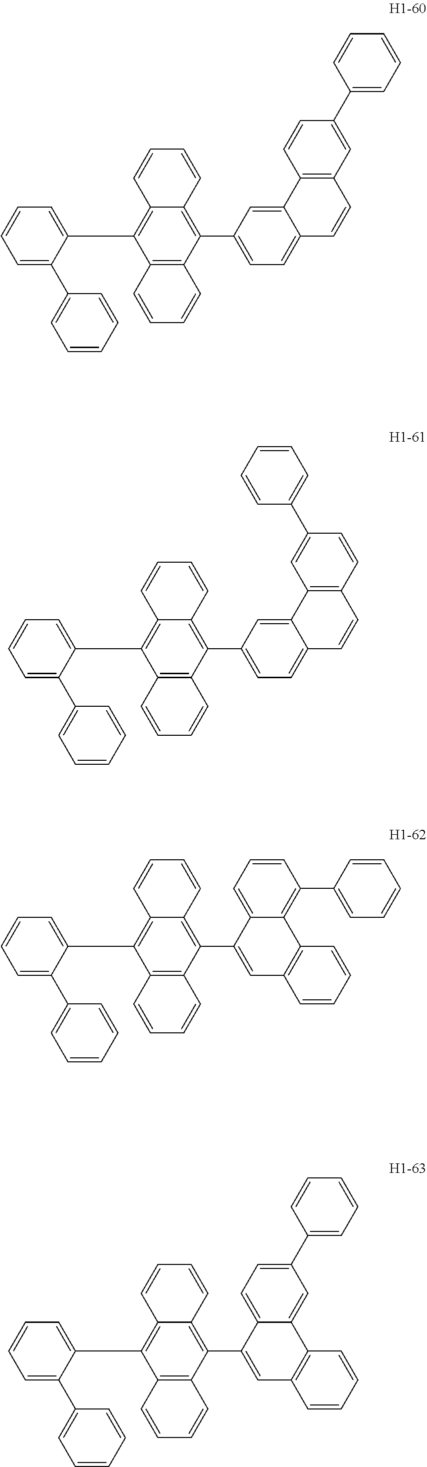

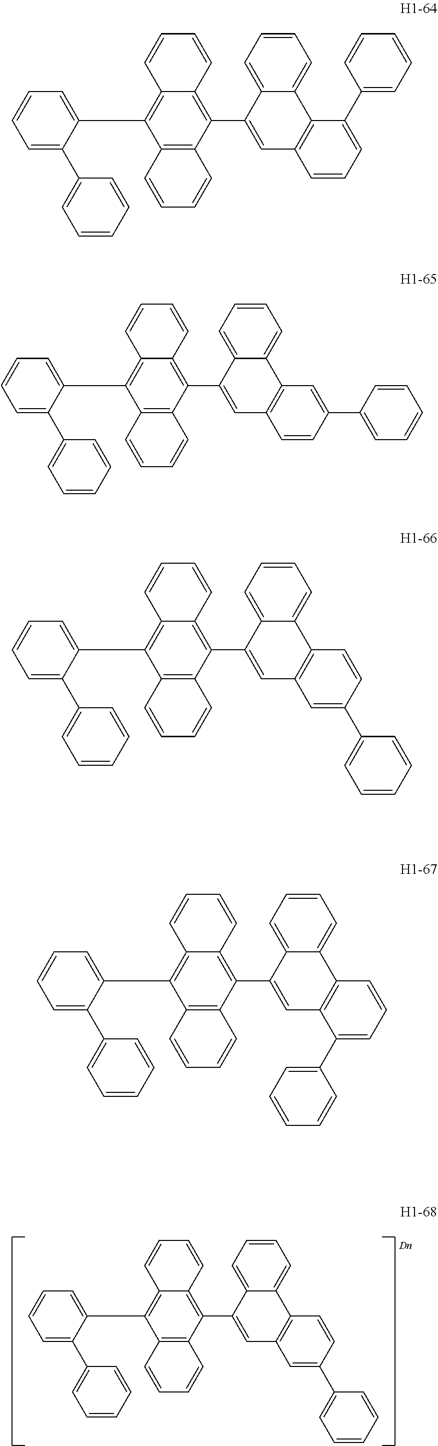

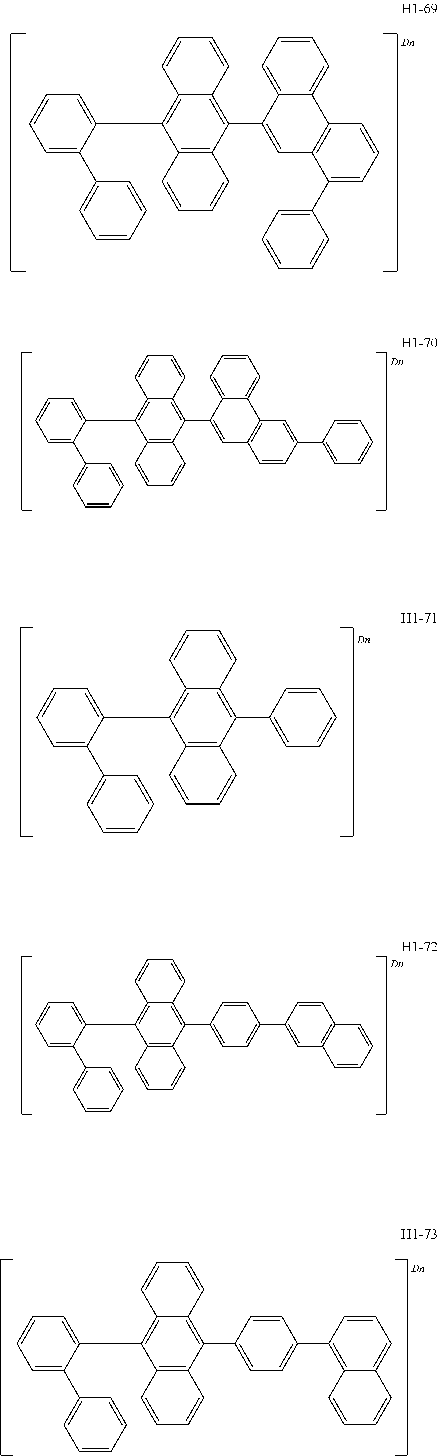

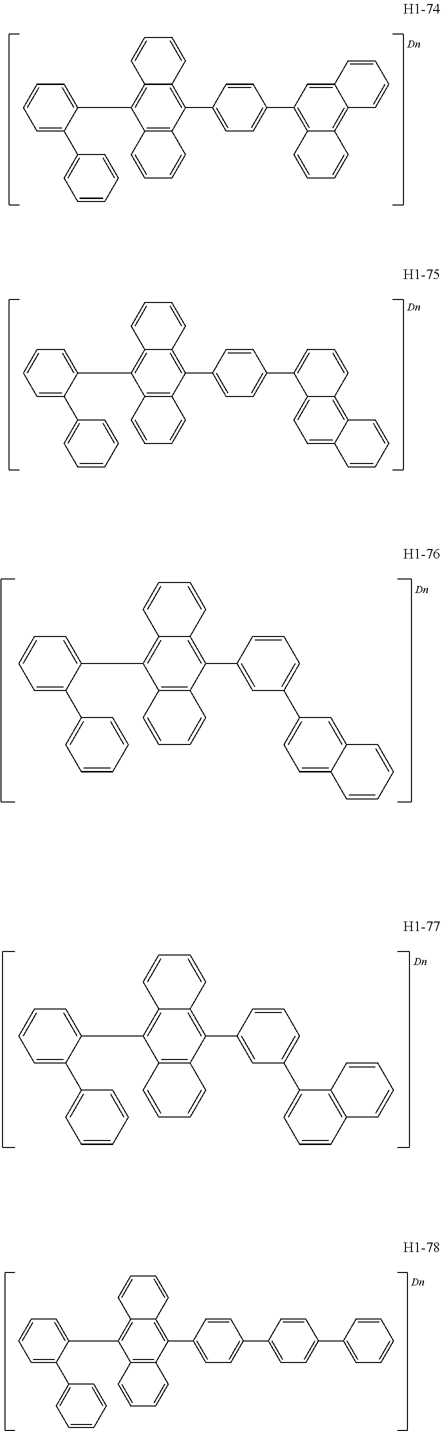

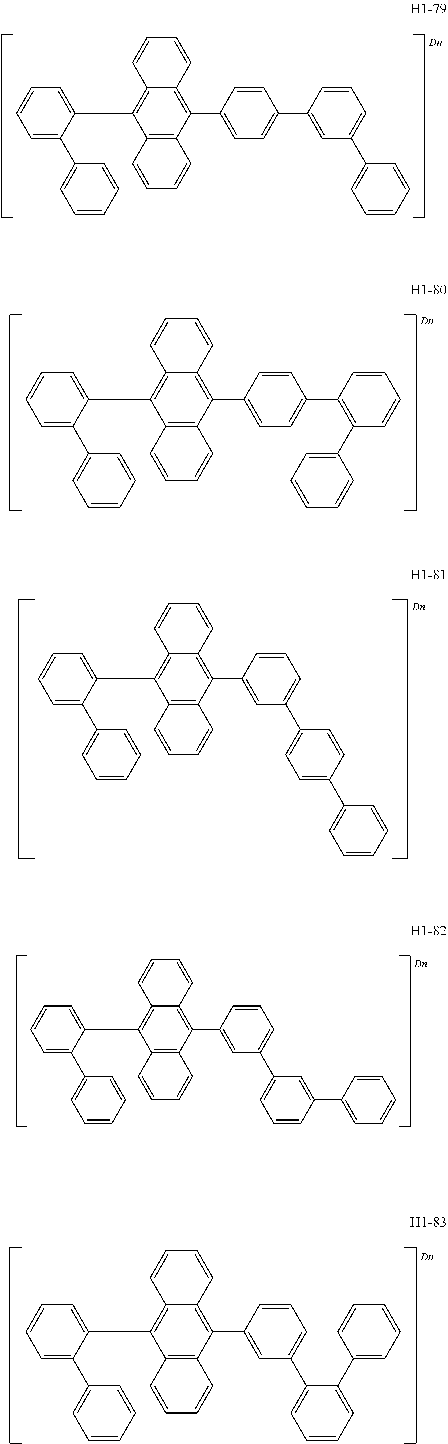

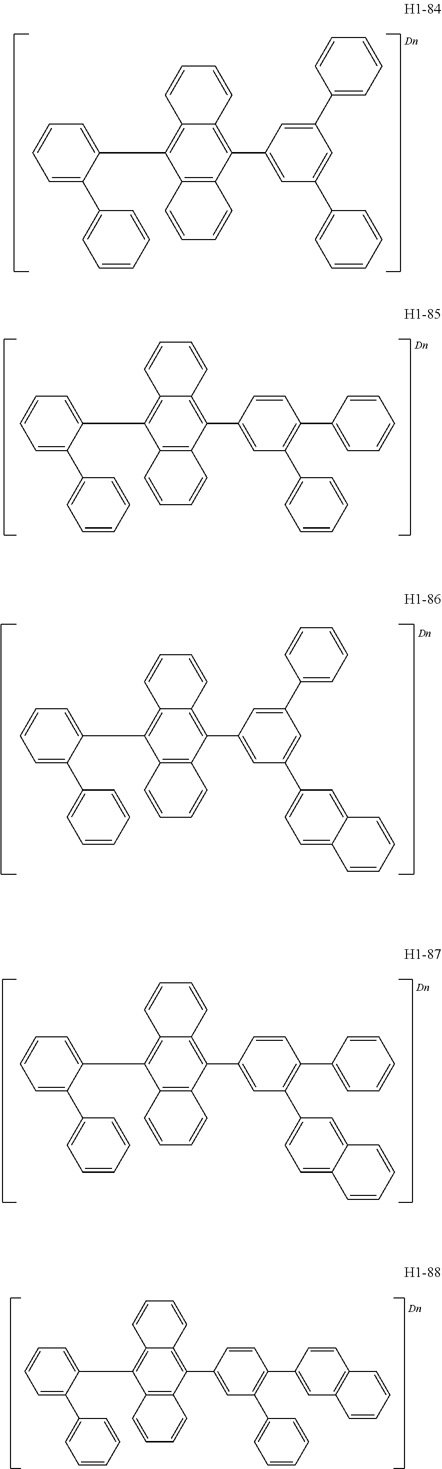

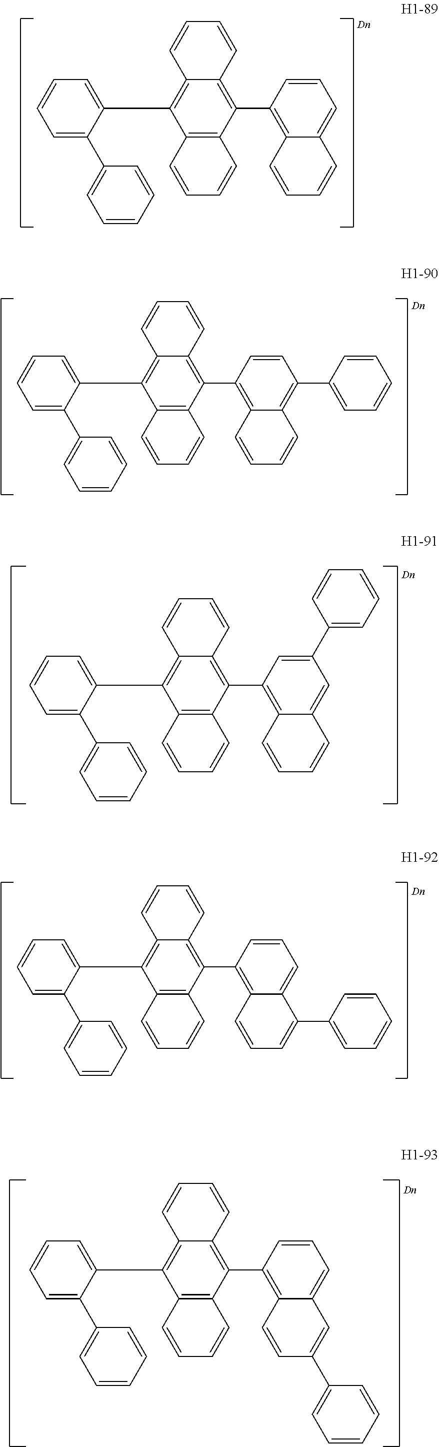

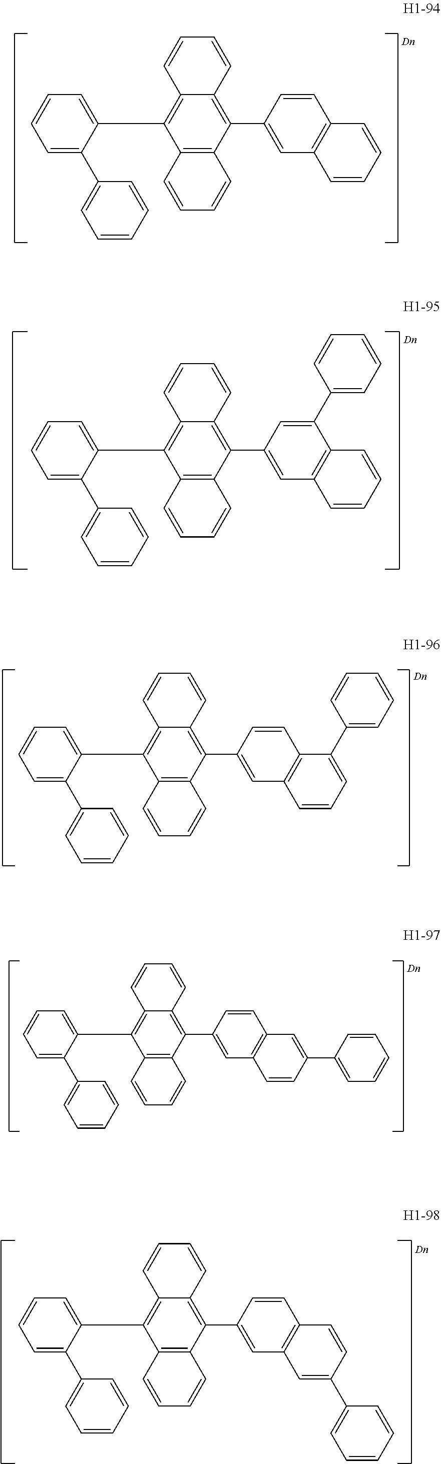

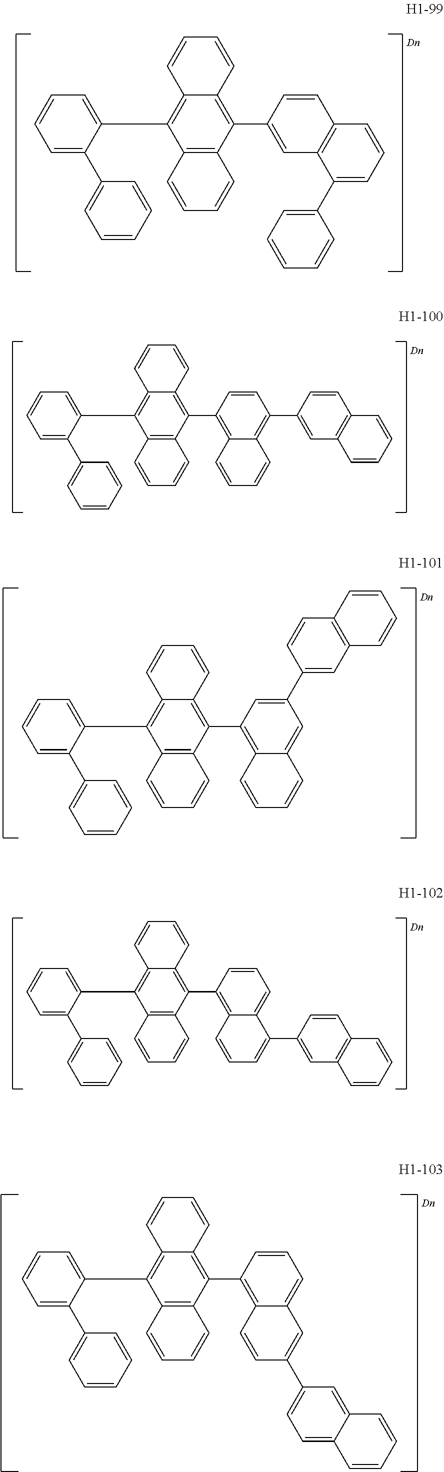

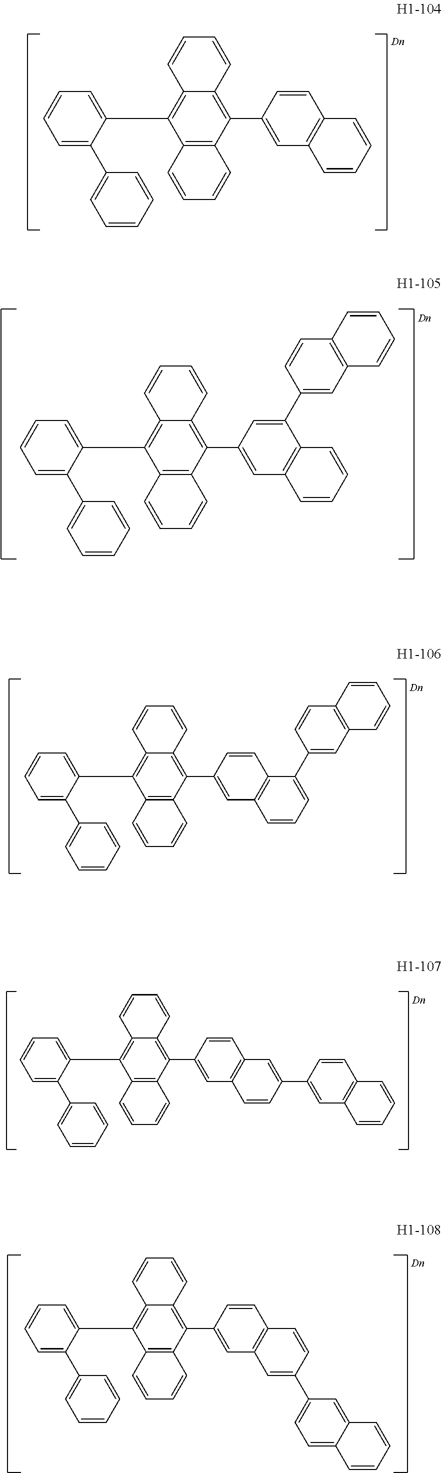

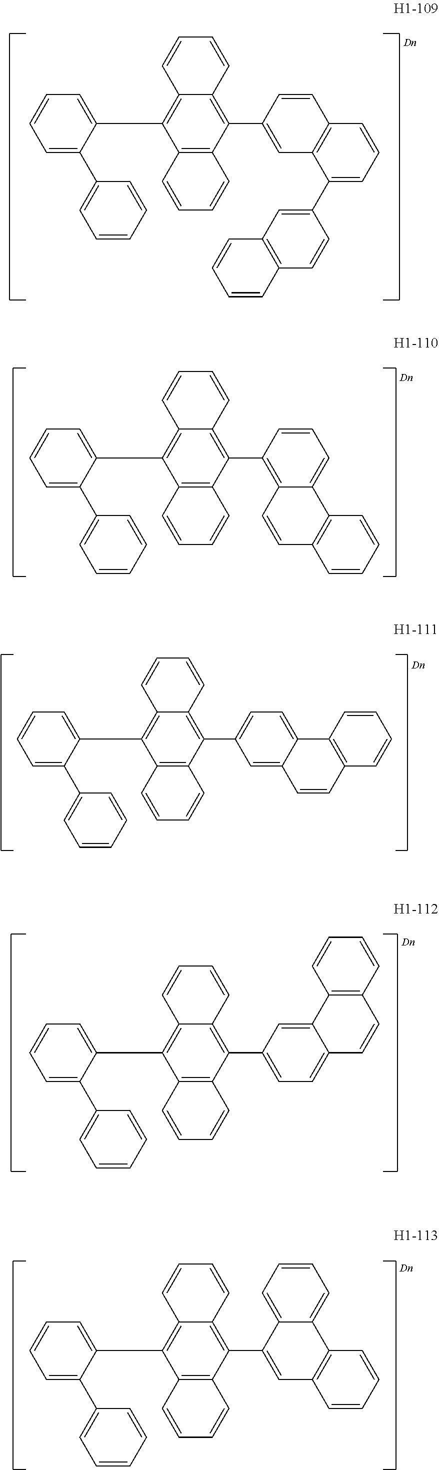

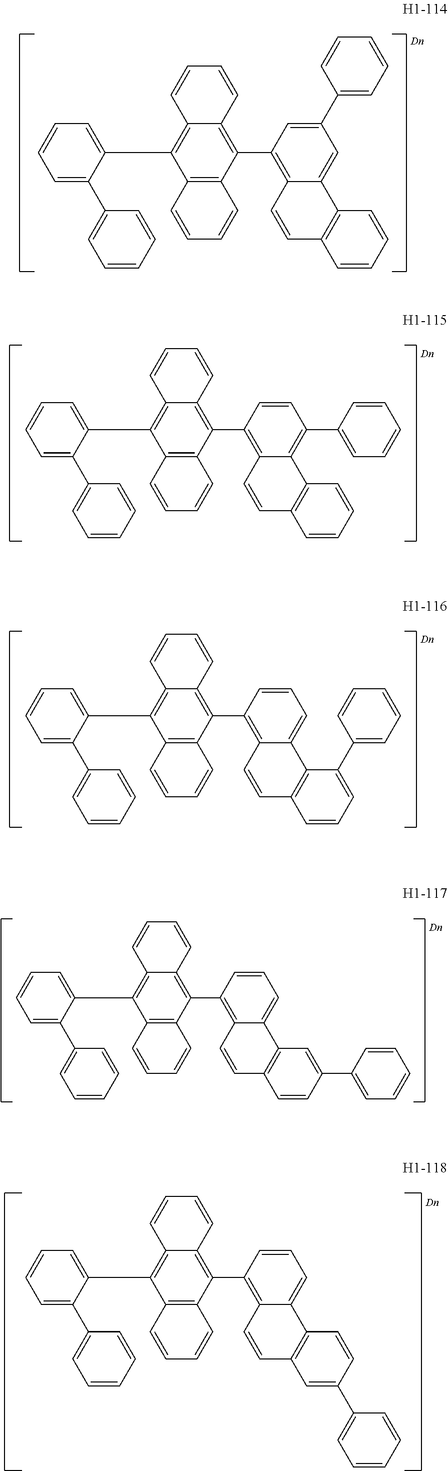

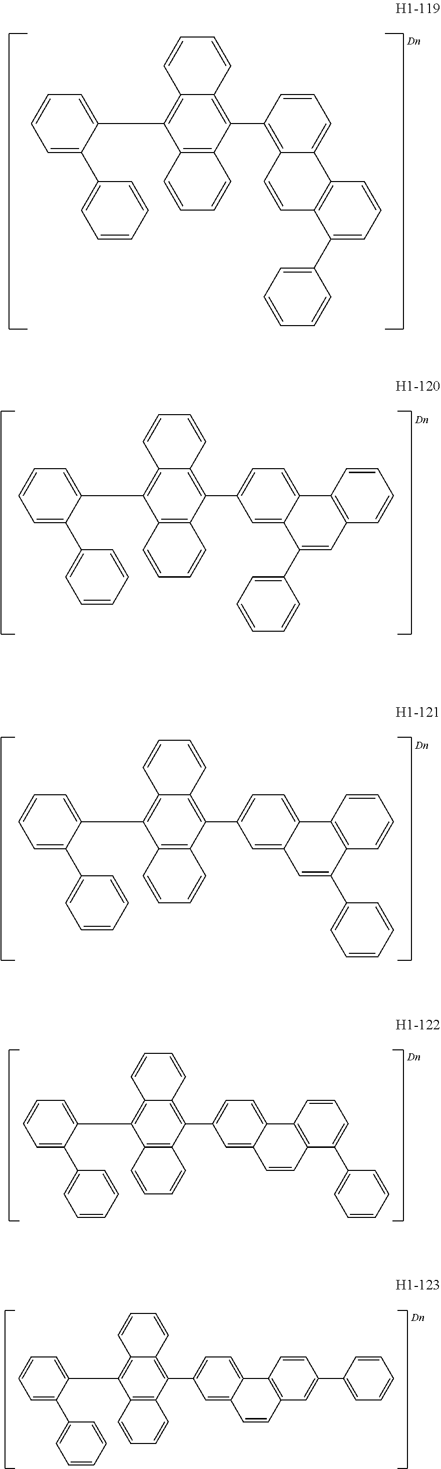

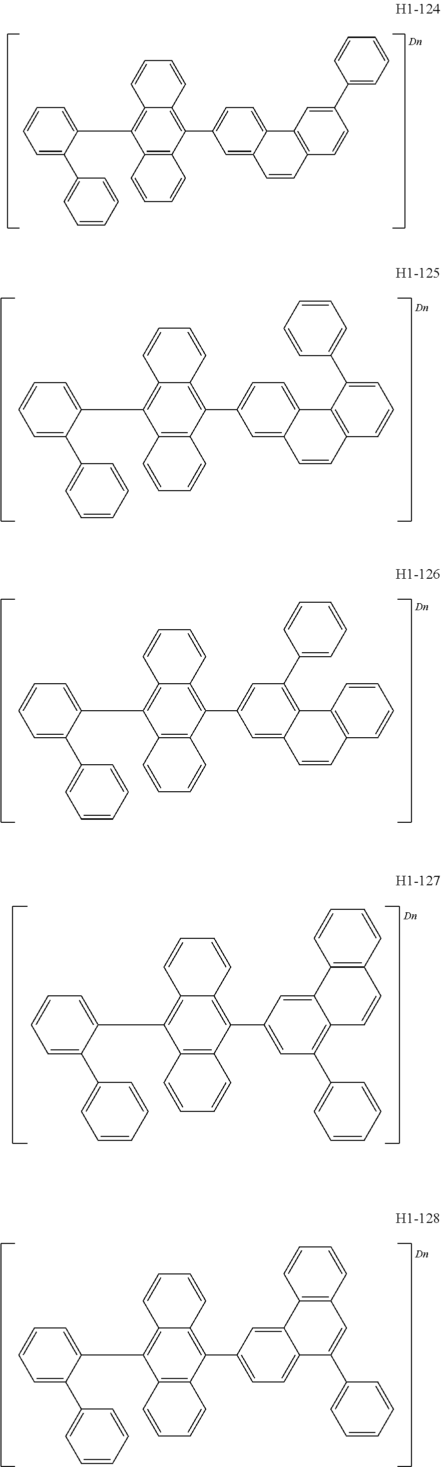

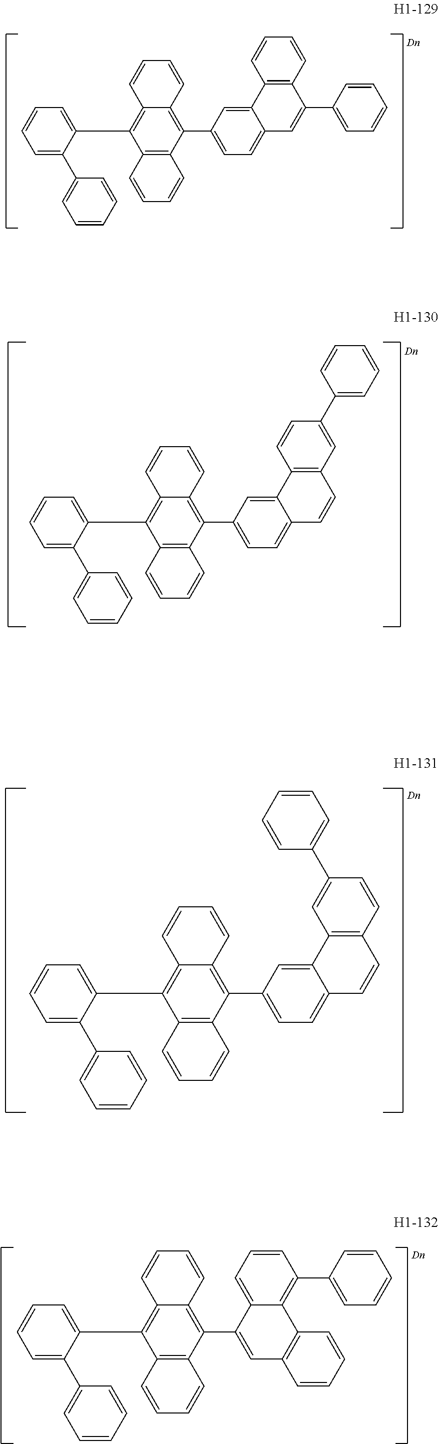

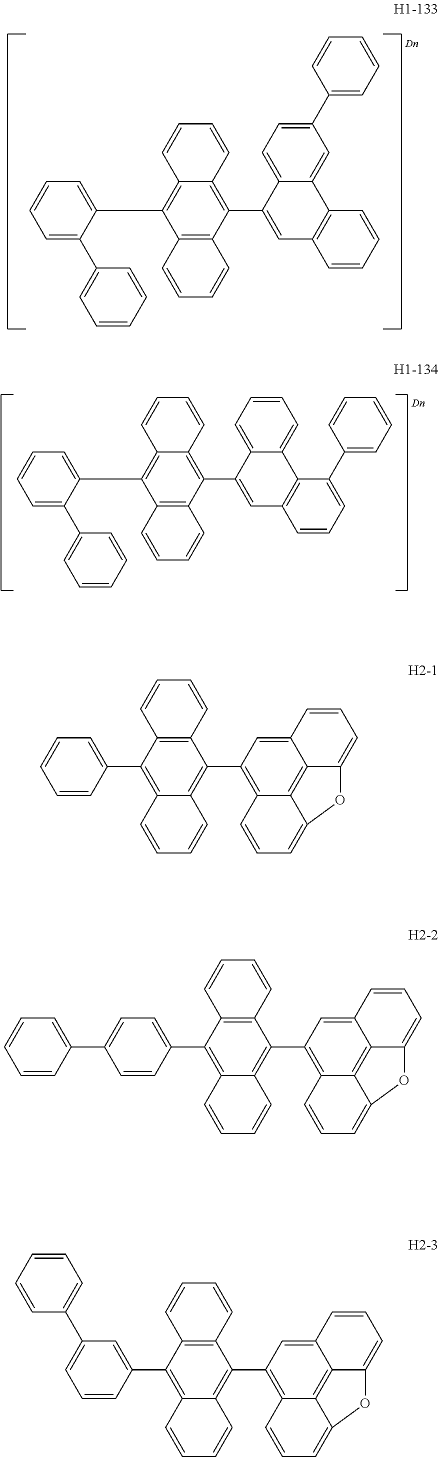

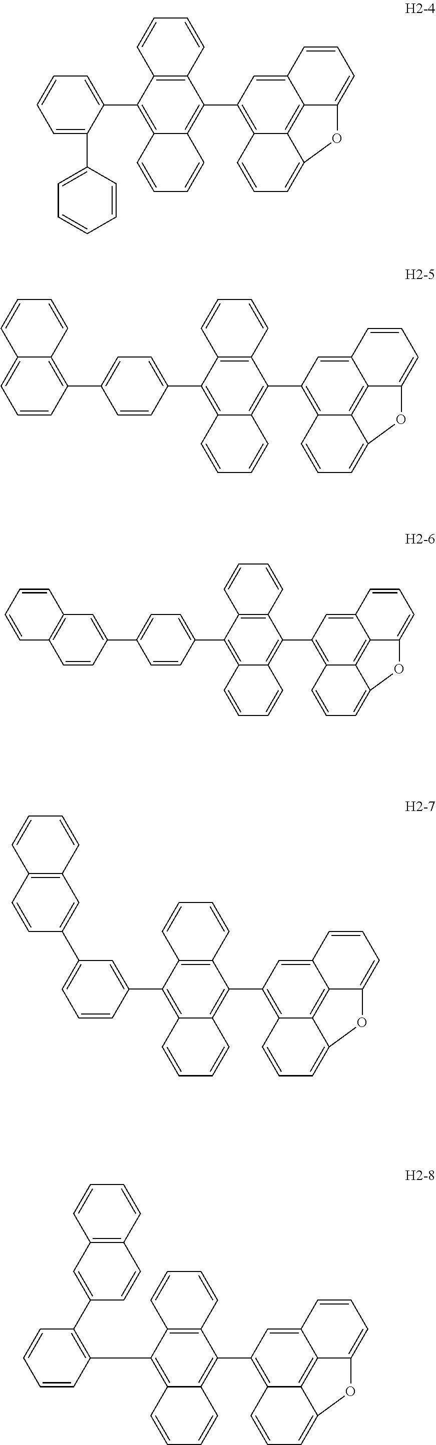 [SMILES: c1ccc(-c2ccccc2-c2c3ccccc3c(-c3cc4cccc5oc6cccc3c6c45)c3ccccc23)cc1 ; c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1 ; c1cc(-c2ccc3ccccc3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccccc3c2)c1]

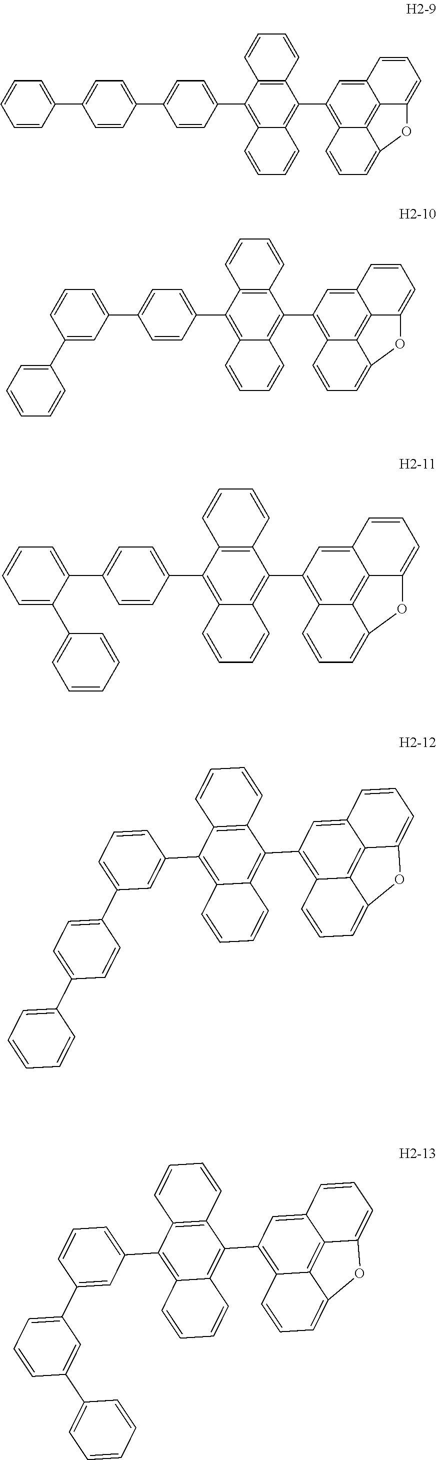 [SMILES: c1ccc(-c2ccc(-c3ccc(-c4c5ccccc5c(-c5cc6cccc7oc8cccc5c8c67)c5ccccc45)cc3)cc2)cc1 ; c1ccc(-c2cccc(-c3ccc(-c4c5ccccc5c(-c5cc6cccc7oc8cccc5c8c67)c5ccccc45)cc3)c2)cc1 ; c1ccc(-c2ccccc2-c2ccc(-c3c4ccccc4c(-c4cc5cccc6oc7cccc4c7c56)c4ccccc34)cc2)cc1 ; c1ccc(-c2ccc(-c3cccc(-c4c5ccccc5c(-c5cc6cccc7oc8cccc5c8c67)c5ccccc45)c3)cc2)cc1 ; c1ccc(-c2cccc(-c3cccc(-c4c5ccccc5c(-c5cc6cccc7oc8cccc5c8c67)c5ccccc45)c3)c2)cc1]

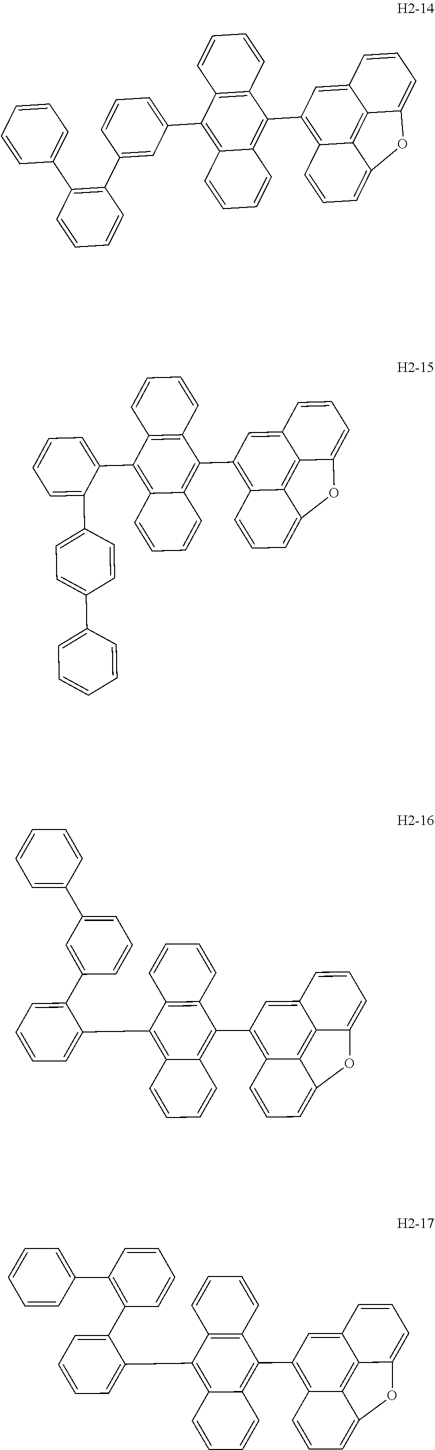 [SMILES: c1ccc(-c2ccccc2-c2cccc(-c3c4ccccc4c(-c4cc5cccc6oc7cccc4c7c56)c4ccccc34)c2)cc1 ; c1ccc(-c2ccc(-c3ccccc3-c3c4ccccc4c(-c4cc5cccc6oc7cccc4c7c56)c4ccccc34)cc2)cc1 ; c1ccc(-c2cccc(-c3ccccc3-c3c4ccccc4c(-c4cc5cccc6oc7cccc4c7c56)c4ccccc34)c2)cc1 ; c1ccc(-c2ccccc2-c2ccccc2-c2c3ccccc3c(-c3cc4cccc5oc6cccc3c6c45)c3ccccc23)cc1]

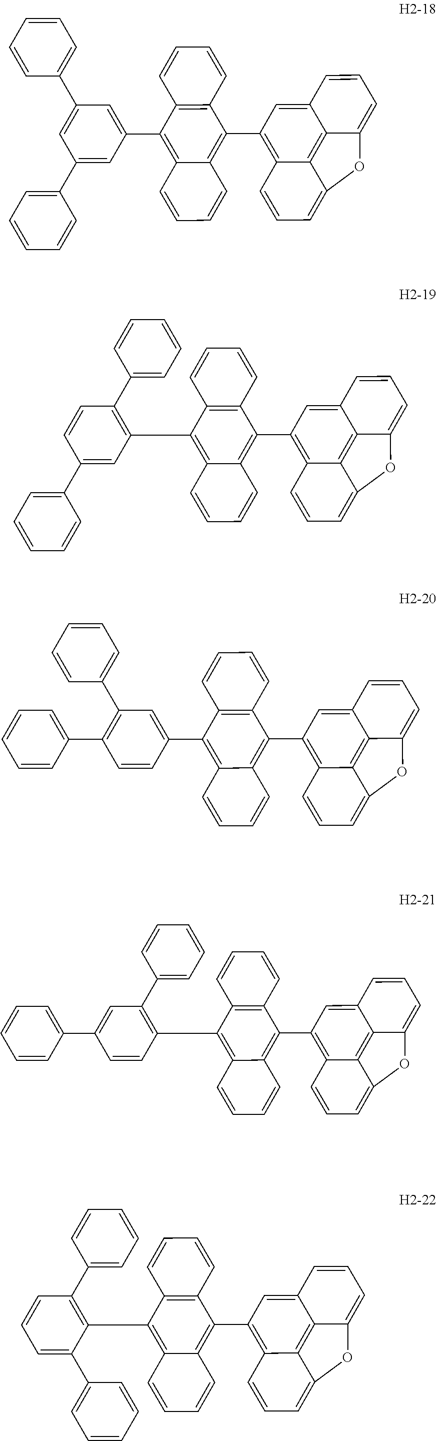 [SMILES: c1ccc(-c2cc(-c3ccccc3)cc(-c3c4ccccc4c(-c4cc5cccc6oc7cccc4c7c56)c4ccccc34)c2)cc1 ; c1ccc(-c2ccc(-c3ccccc3)c(-c3c4ccccc4c(-c4cc5cccc6oc7cccc4c7c56)c4ccccc34)c2)cc1 ; c1ccc(-c2ccc(-c3c4ccccc4c(-c4cc5cccc6oc7cccc4c7c56)c4ccccc34)cc2-c2ccccc2)cc1 ; c1ccc(-c2ccc(-c3c4ccccc4c(-c4cc5cccc6oc7cccc4c7c56)c4ccccc34)c(-c3ccccc3)c2)cc1 ; c1ccc(-c2cccc(-c3ccccc3)c2-c2c3ccccc3c(-c3cc4cccc5oc6cccc3c6c45)c3ccccc23)cc1]

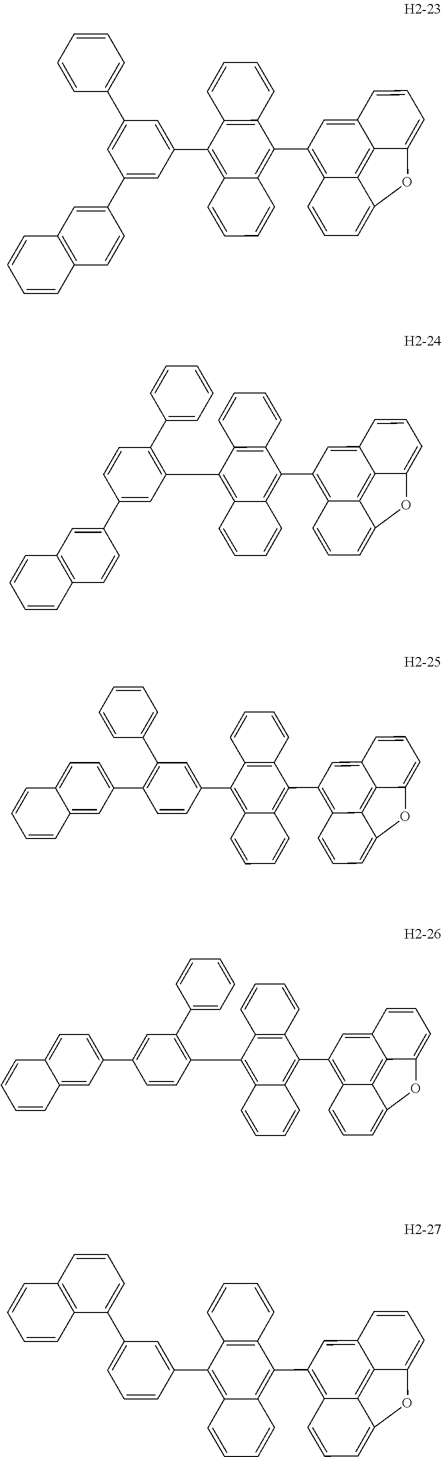 [SMILES: c1ccc(-c2cc(-c3ccc4ccccc4c3)cc(-c3c4ccccc4c(-c4cc5cccc6oc7cccc4c7c56)c4ccccc34)c2)cc1 ; c1ccc(-c2ccc(-c3ccc4ccccc4c3)cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)ccc2-c2ccc3ccccc3c2)cc1 ; c1ccc(-c2cc(-c3ccc4ccccc4c3)ccc2-c2c3ccccc3c(-c3cc4cccc5oc6cccc3c6c45)c3ccccc23)cc1 ; c1cc(-c2cccc3ccccc23)cc(-c2c3ccccc3c(-c3cc4cccc5oc6cccc3c6c45)c3ccccc23)c1]

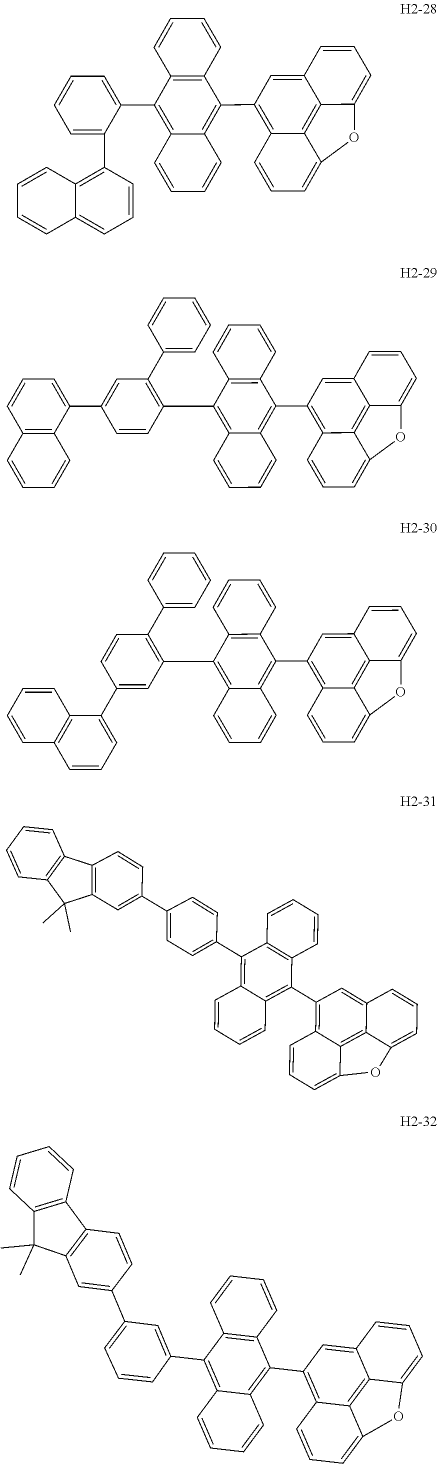 [SMILES: c1ccc(-c2c3ccccc3c(-c3cc4cccc5oc6cccc3c6c45)c3ccccc23)c(-c2cccc3ccccc23)c1 ; c1ccc(-c2cc(-c3cccc4ccccc34)ccc2-c2c3ccccc3c(-c3cc4cccc5oc6cccc3c6c45)c3ccccc23)cc1 ; c1ccc(-c2ccc(-c3cccc4ccccc34)cc2-c2c3ccccc3c(-c3cc4cccc5oc6cccc3c6c45)c3ccccc23)cc1 ; CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21]

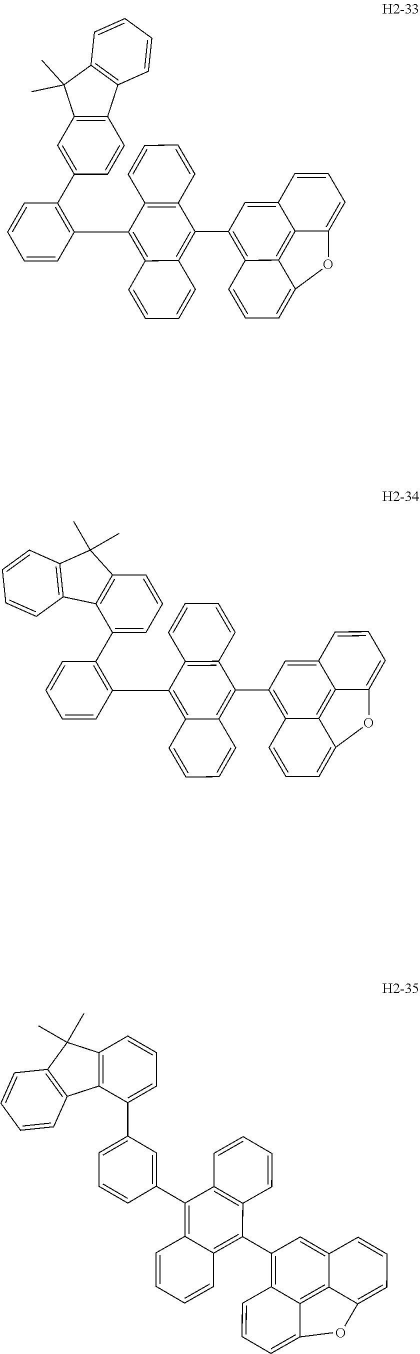 [SMILES: CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21]

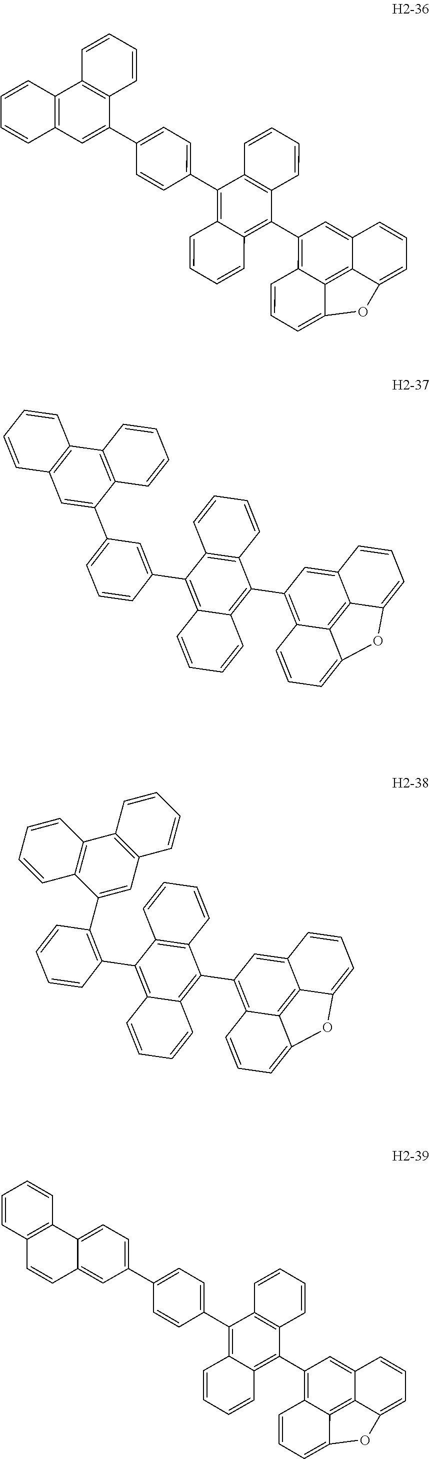 [SMILES: c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1 ; c1ccc2c(c1)ccc1cc(-c3ccc(-c4c5ccccc5c(-c5cc6cccc7oc8cccc5c8c67)c5ccccc45)cc3)ccc12]

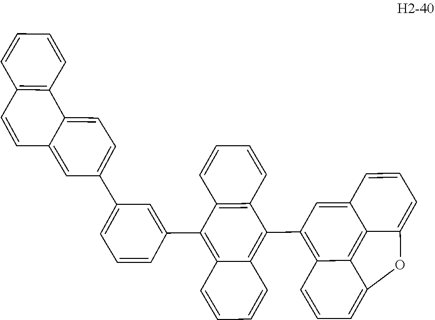 [SMILES: c1cc(-c2ccc3c(ccc4ccccc43)c2)cc(-c2c3ccccc3c(-c3cc4cccc5oc6cccc3c6c45)c3ccccc23)c1]

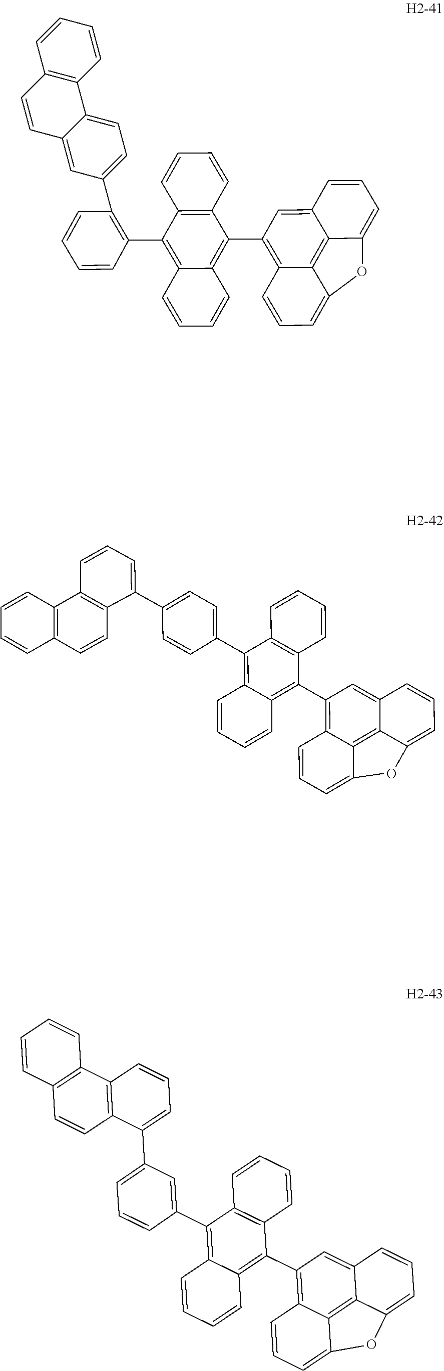 [SMILES: c1ccc(-c2c3ccccc3c(-c3cc4cccc5oc6cccc3c6c45)c3ccccc23)c(-c2ccc3c(ccc4ccccc43)c2)c1 ; c1ccc2c(c1)ccc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2ccc2ccccc23)c1]

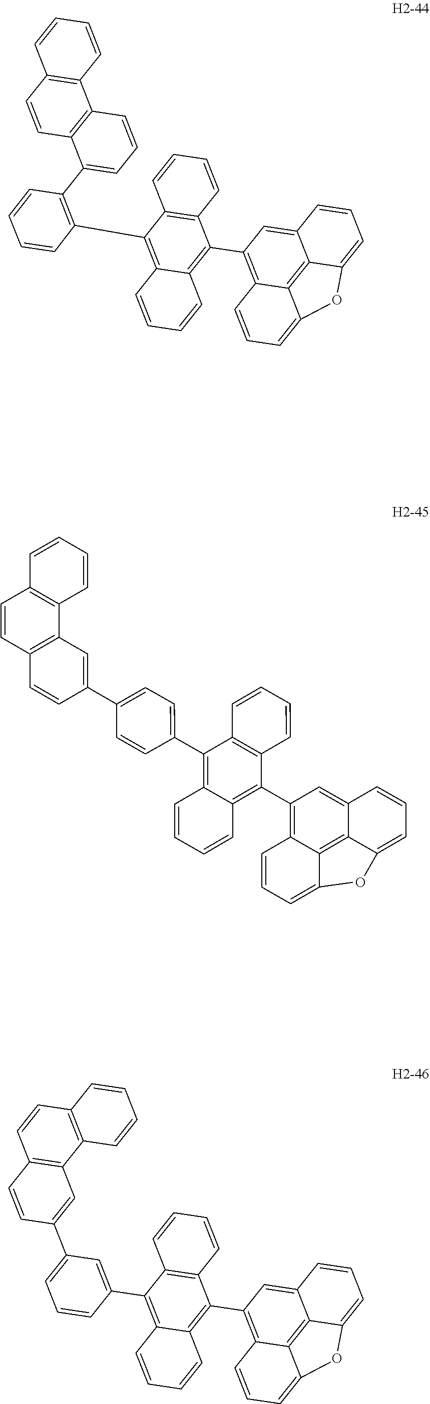 [SMILES: c1ccc(-c2cccc3c2ccc2ccccc23)c(-c2c3ccccc3c(-c3cc4cccc5oc6cccc3c6c45)c3ccccc23)c1 ; c1ccc2c(c1)ccc1ccc(-c3ccc(-c4c5ccccc5c(-c5cc6cccc7oc8cccc5c8c67)c5ccccc45)cc3)cc12 ; c1cc(-c2ccc3ccc4ccccc4c3c2)cc(-c2c3ccccc3c(-c3cc4cccc5oc6cccc3c6c45)c3ccccc23)c1]

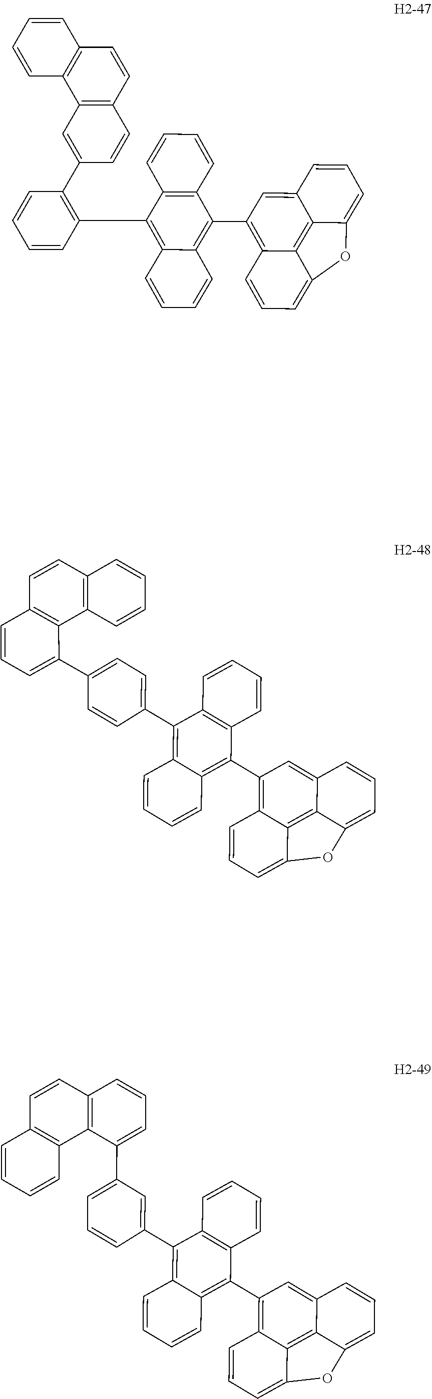 [SMILES: c1ccc(-c2c3ccccc3c(-c3cc4cccc5oc6cccc3c6c45)c3ccccc23)c(-c2ccc3ccc4ccccc4c3c2)c1 ; c1ccc2c(c1)ccc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3ccc4ccccc4c23)c1]

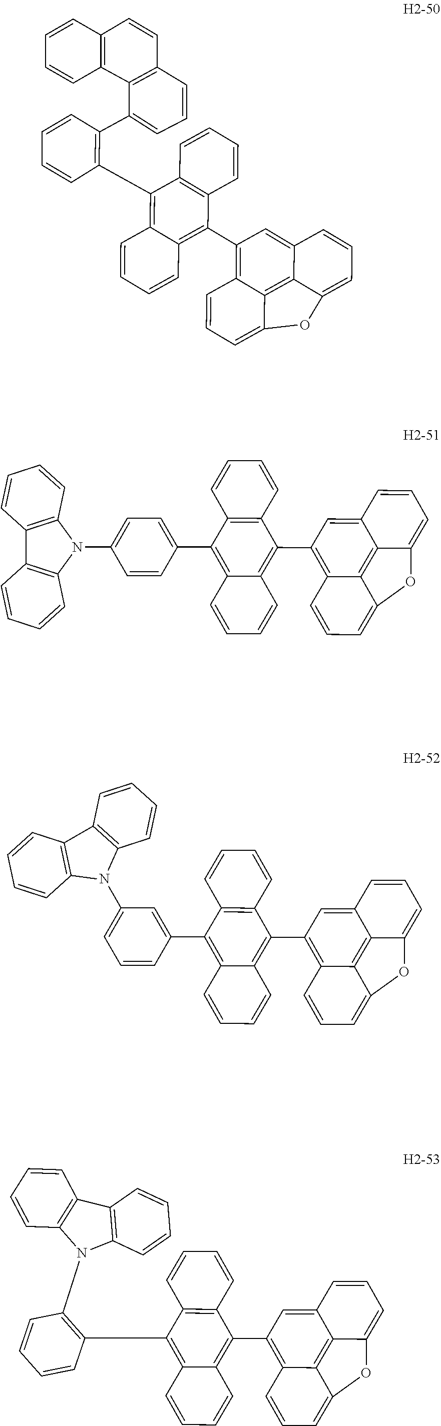 [SMILES: c1ccc(-c2cccc3ccc4ccccc4c23)c(-c2c3ccccc3c(-c3cc4cccc5oc6cccc3c6c45)c3ccccc23)c1 ; c1cc2cc(-c3c4ccccc4c(-c4ccc(-n5c6ccccc6c6ccccc65)cc4)c4ccccc34)c3cccc4oc(c1)c2c43 ; c1cc(-c2c3ccccc3c(-c3cc4cccc5oc6cccc3c6c45)c3ccccc23)cc(-n2c3ccccc3c3ccccc32)c1 ; c1ccc(-n2c3ccccc3c3ccccc32)c(-c2c3ccccc3c(-c3cc4cccc5oc6cccc3c6c45)c3ccccc23)c1]

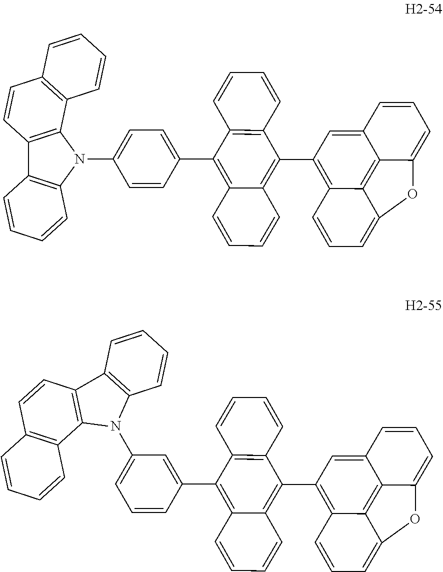 [SMILES: c1ccc2c(c1)ccc1c3ccccc3n(-c3ccc(-c4c5ccccc5c(-c5cc6cccc7oc8cccc5c8c67)c5ccccc45)cc3)c21 ; c1cc(-c2c3ccccc3c(-c3cc4cccc5oc6cccc3c6c45)c3ccccc23)cc(-n2c3ccccc3c3ccc4ccccc4c32)c1]

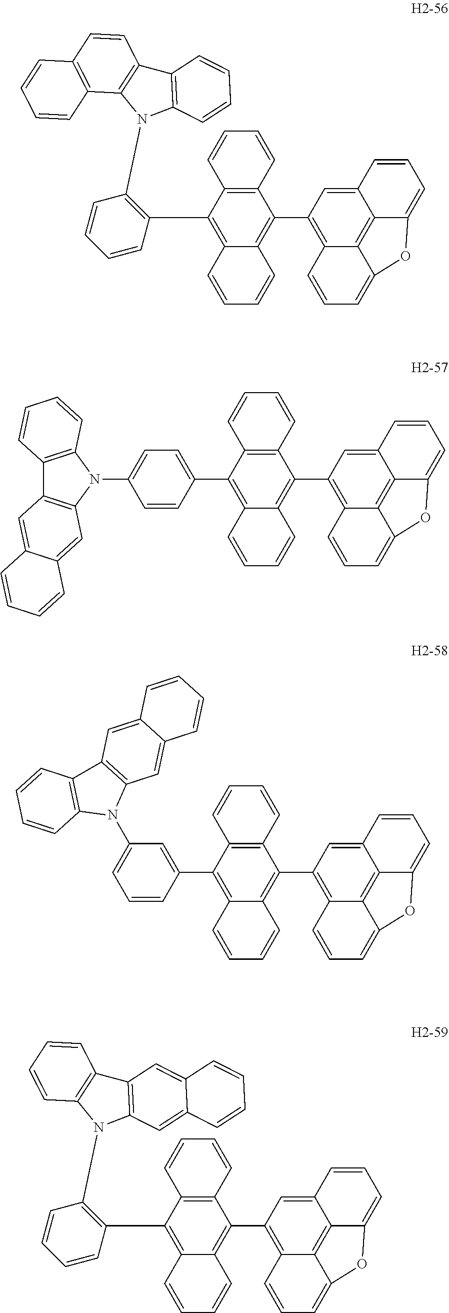 [SMILES: c1ccc(-n2c3ccccc3c3ccc4ccccc4c32)c(-c2c3ccccc3c(-c3cc4cccc5oc6cccc3c6c45)c3ccccc23)c1 ; c1ccc2cc3c(cc2c1)c1ccccc1n3-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3cc4ccccc4cc32)c1 ; c1ccc(-n2c3ccccc3c3cc4ccccc4cc32)c(-c2c3ccccc3c(-c3cc4cccc5oc6cccc3c6c45)c3ccccc23)c1]

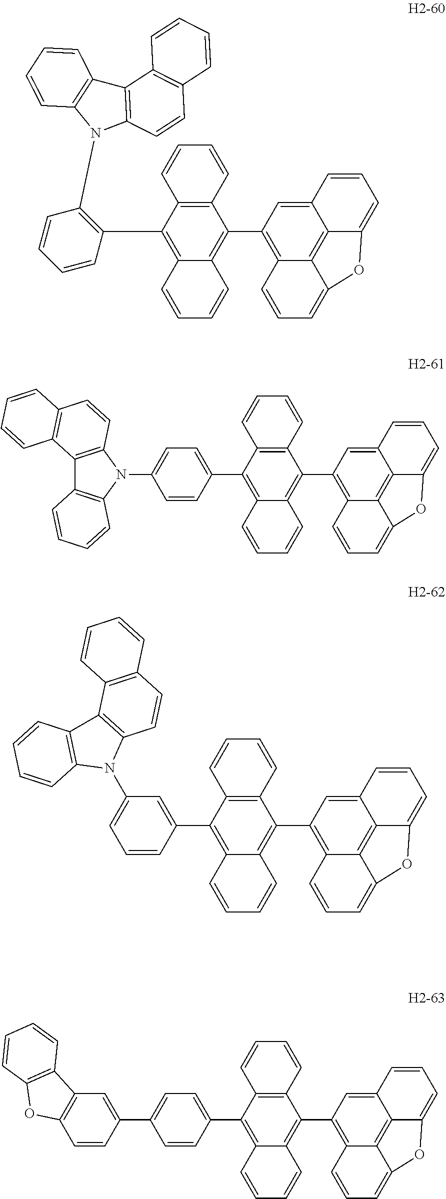 [SMILES: c1ccc(-n2c3ccccc3c3c4ccccc4ccc32)c(-c2c3ccccc3c(-c3cc4cccc5oc6cccc3c6c45)c3ccccc23)c1 ; c1ccc2c(c1)ccc1c2c2ccccc2n1-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3c4ccccc4ccc32)c1 ; c1ccc2c(c1)oc1ccc(-c3ccc(-c4c5ccccc5c(-c5cc6cccc7oc8cccc5c8c67)c5ccccc45)cc3)cc12]

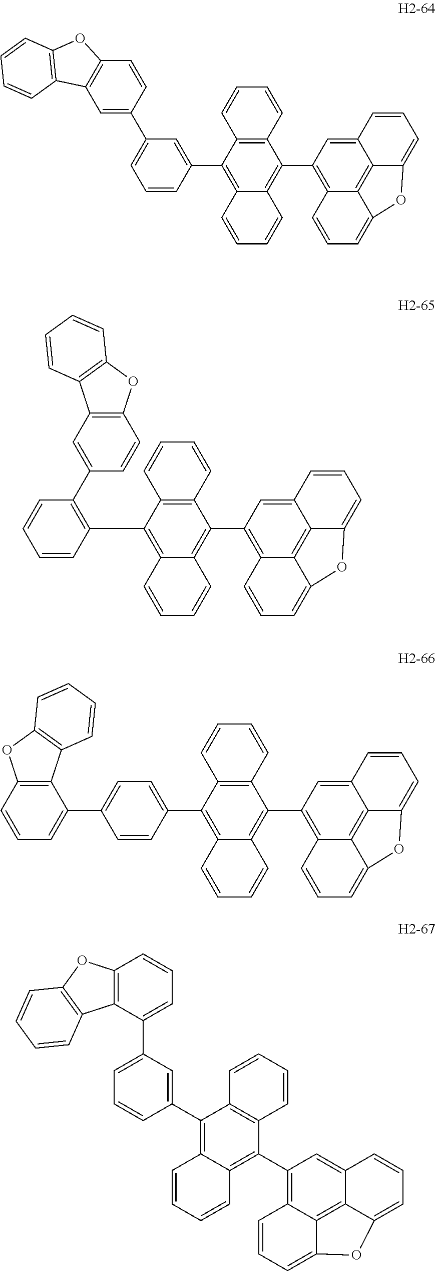 [SMILES: c1cc(-c2ccc3oc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3oc4ccccc4c3c2)c1 ; c1ccc2c(c1)oc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3oc4ccccc4c23)c1]

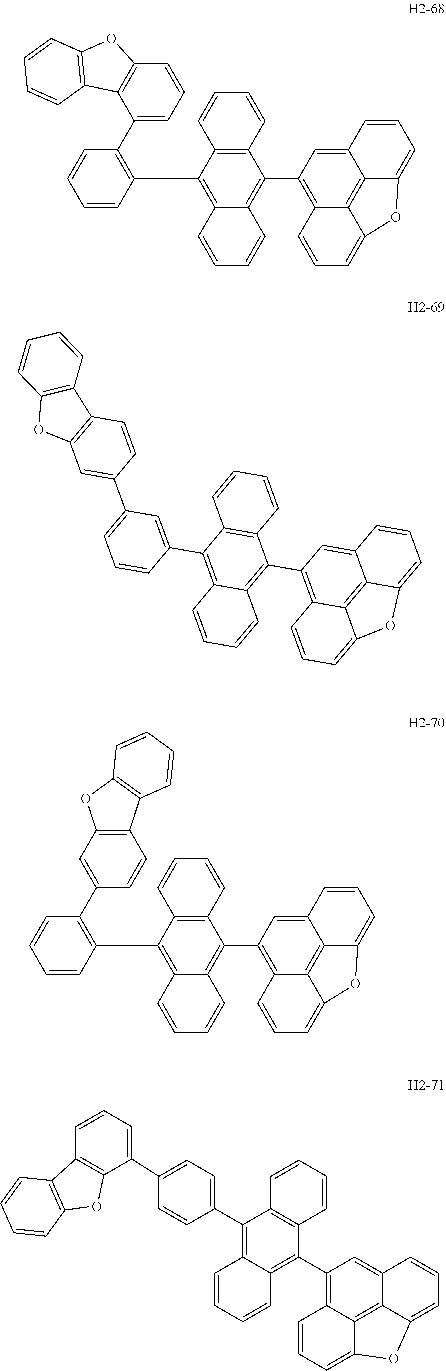 [SMILES: c1ccc(-c2cccc3oc4ccccc4c23)c(-c2c3ccccc3c(-c3cc4cccc5oc6cccc3c6c45)c3ccccc23)c1 ; c1cc(-c2ccc3c(c2)oc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7oc8cccc5c8c67)c5ccccc45)cc3)cccc12]

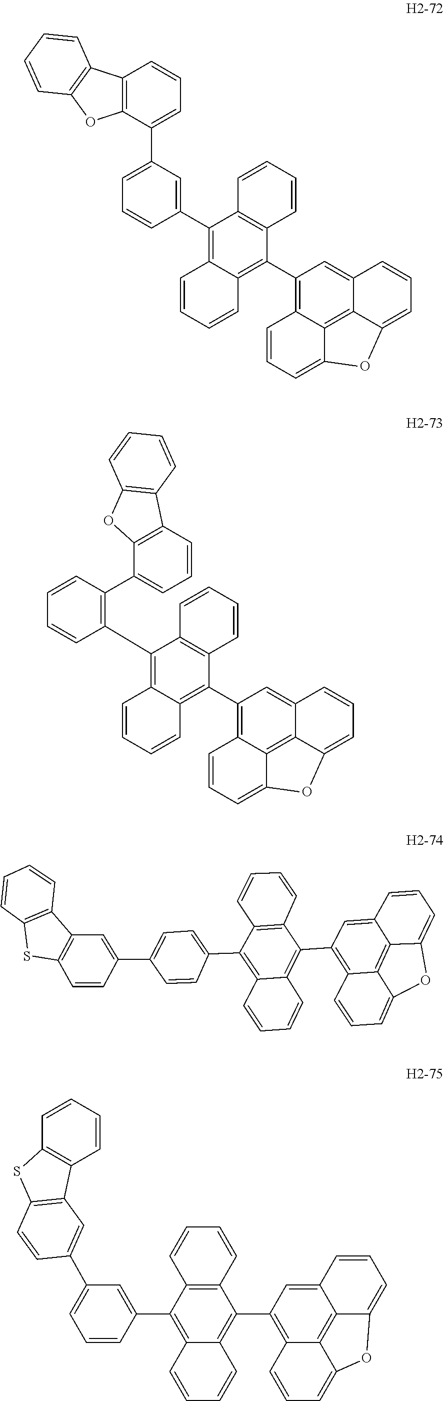 [SMILES: c1cc(-c2c3ccccc3c(-c3cc4cccc5oc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5oc6cccc3c6c45)c3ccccc23)c1 ; c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1]

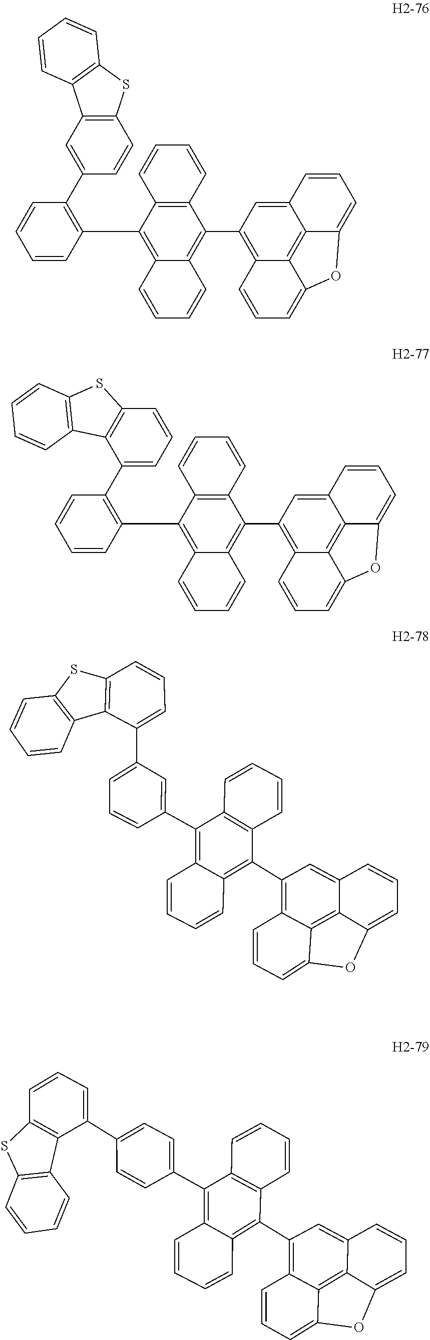 [SMILES: c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1 ; c1cc2cc(-c3c4ccccc4c(-c4ccc(-c5cccc6sc7ccccc7c56)cc4)c4ccccc34)c3cccc4oc(c1)c2c43]

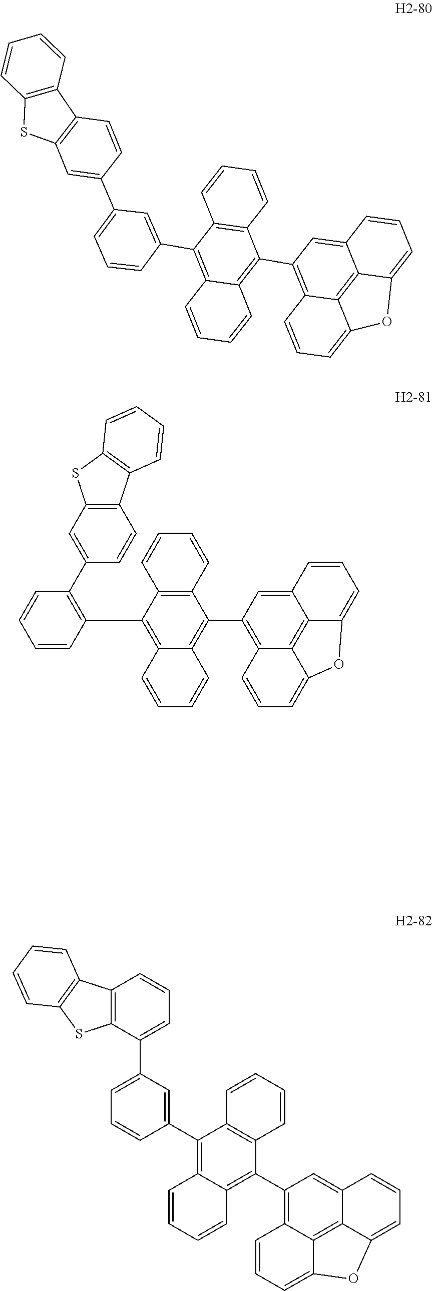 [SMILES: c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1]

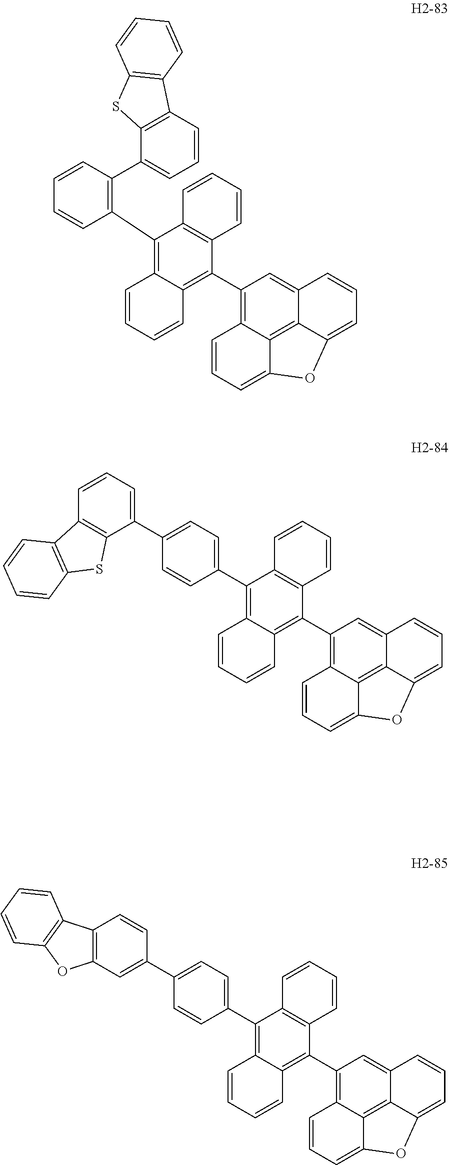 [SMILES: c1ccc(-c2cccc3c2sc2ccccc23)c(-c2c3ccccc3c(-c3cc4cccc5oc6cccc3c6c45)c3ccccc23)c1 ; c1cc2cc(-c3c4ccccc4c(-c4ccc(-c5cccc6c5sc5ccccc56)cc4)c4ccccc34)c3cccc4oc(c1)c2c43 ; c1ccc2c(c1)oc1cc(-c3ccc(-c4c5ccccc5c(-c5cc6cccc7oc8cccc5c8c67)c5ccccc45)cc3)ccc12]

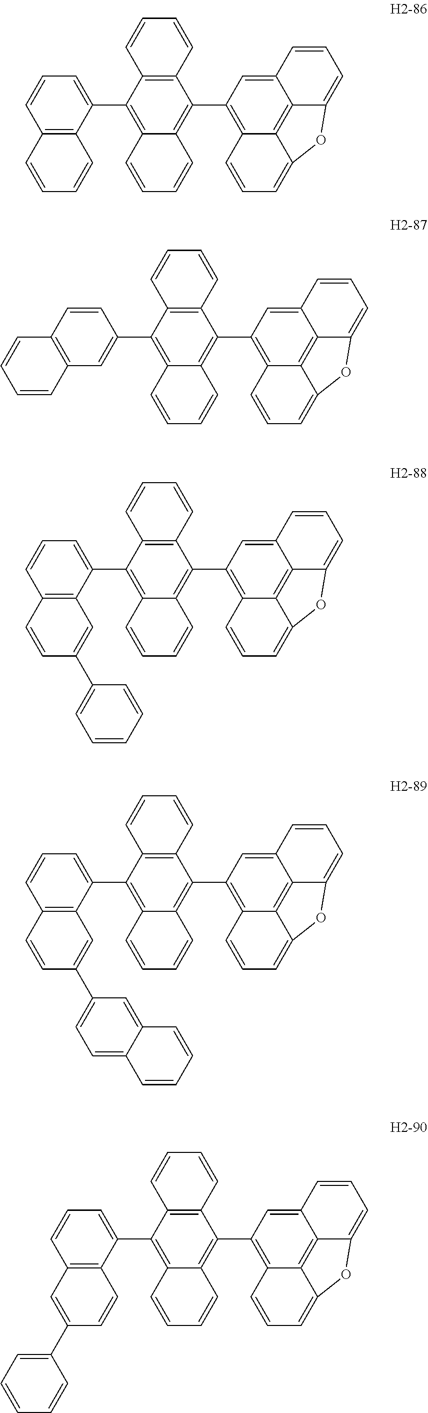 [SMILES: c1ccc2c(-c3c4ccccc4c(-c4cc5cccc6oc7cccc4c7c56)c4ccccc34)cccc2c1 ; c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1]

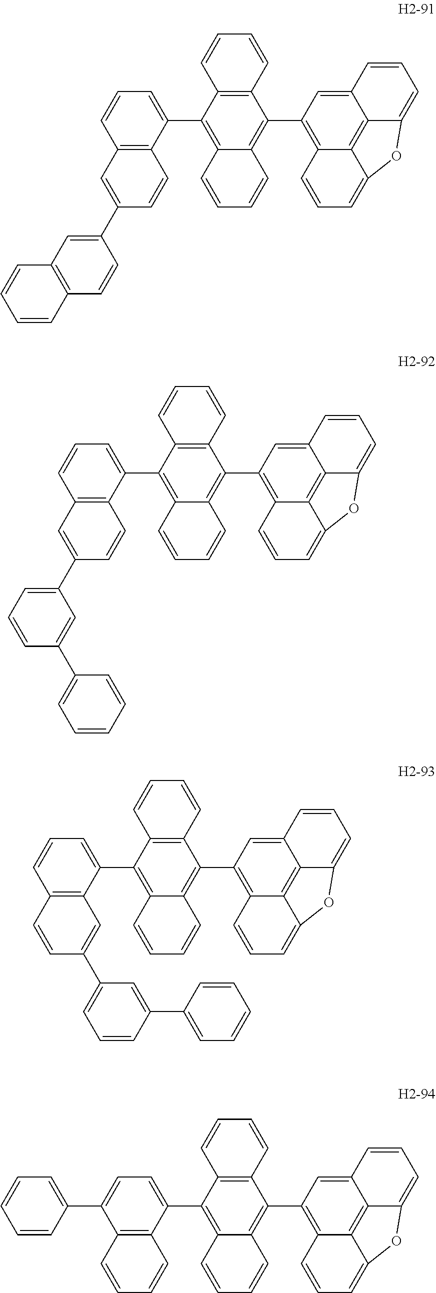 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1]

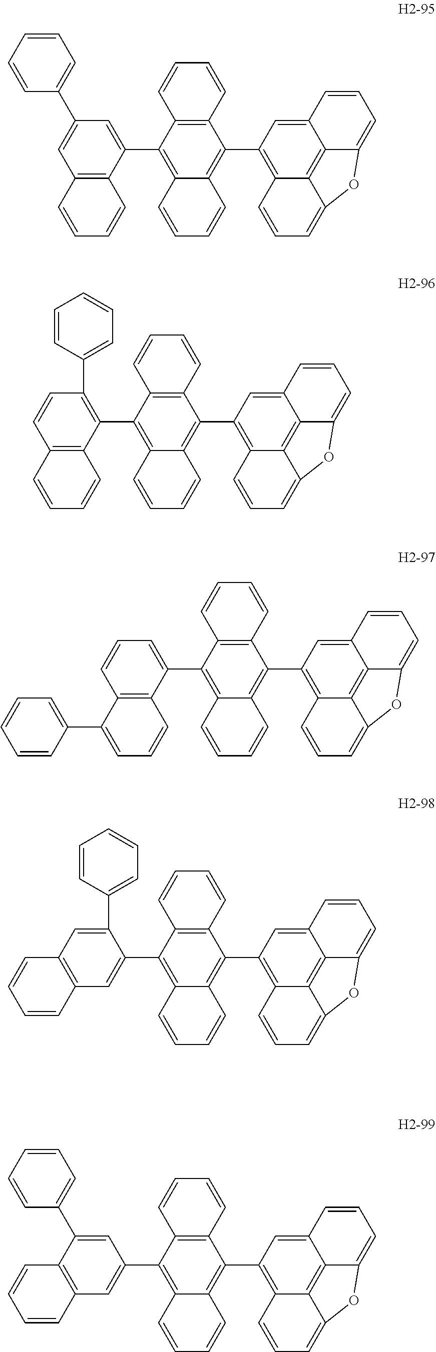 [SMILES: c1ccc(-c2cc(-c3c4ccccc4c(-c4cc5cccc6oc7cccc4c7c56)c4ccccc34)c3ccccc3c2)cc1 ; c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1]

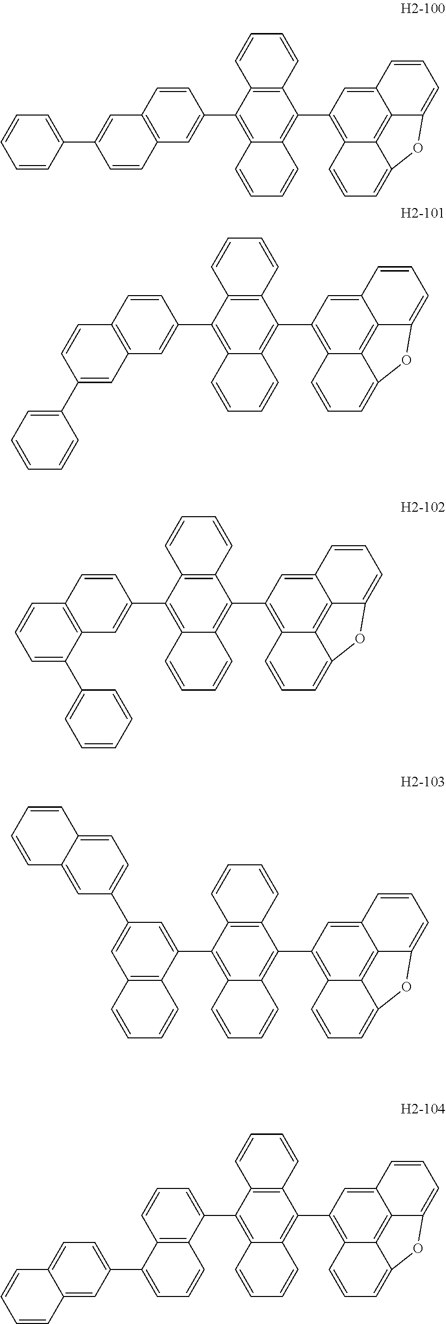

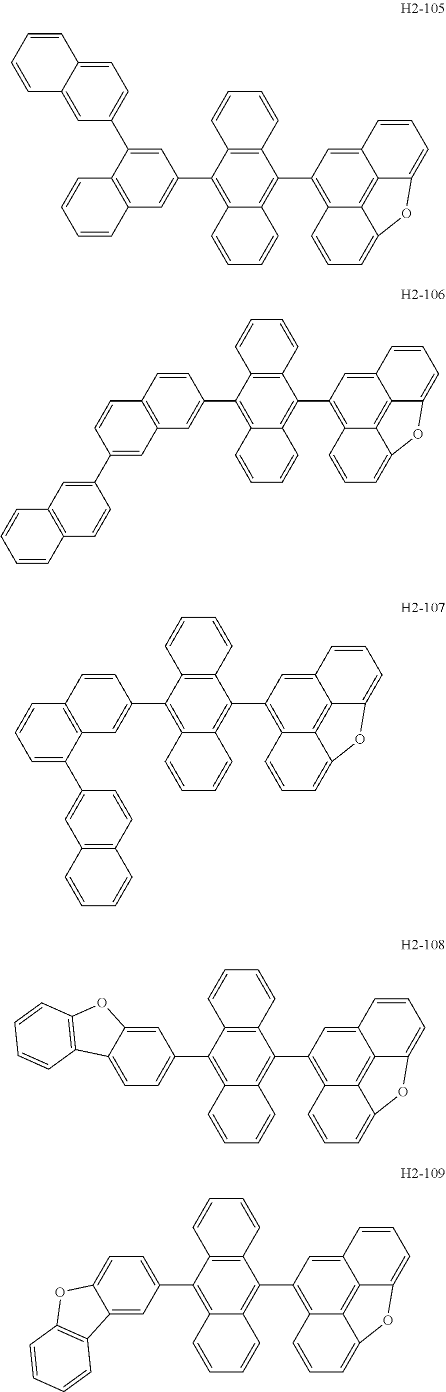 [SMILES: c1ccc2cc(-c3cc(-c4c5ccccc5c(-c5cc6cccc7oc8cccc5c8c67)c5ccccc45)cc4ccccc34)ccc2c1 ; c1ccc2cc(-c3ccc4ccc(-c5c6ccccc6c(-c6cc7cccc8oc9cccc6c9c78)c6ccccc56)cc4c3)ccc2c1 ; c1ccc2cc(-c3cccc4ccc(-c5c6ccccc6c(-c6cc7cccc8oc9cccc6c9c78)c6ccccc56)cc34)ccc2c1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12]

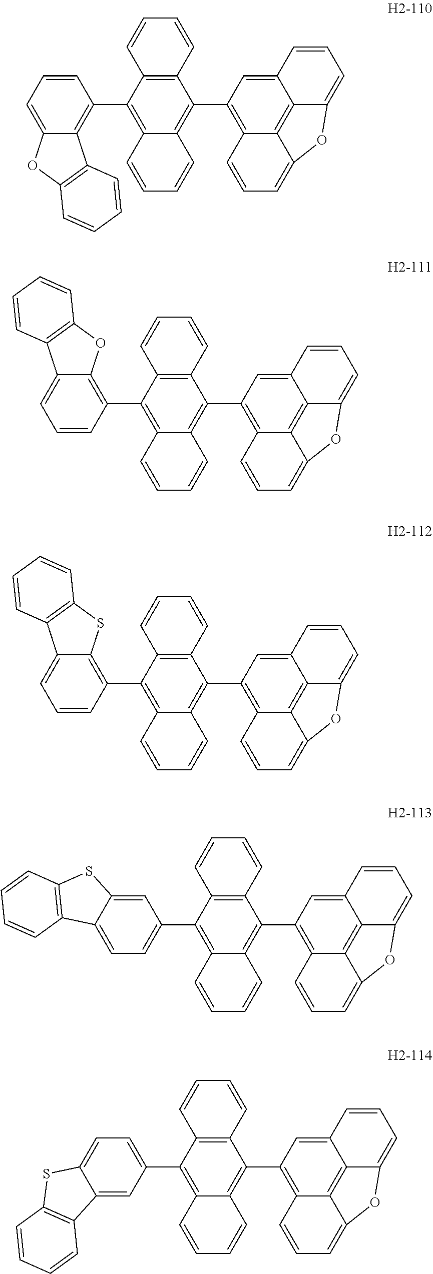 [SMILES: c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43]

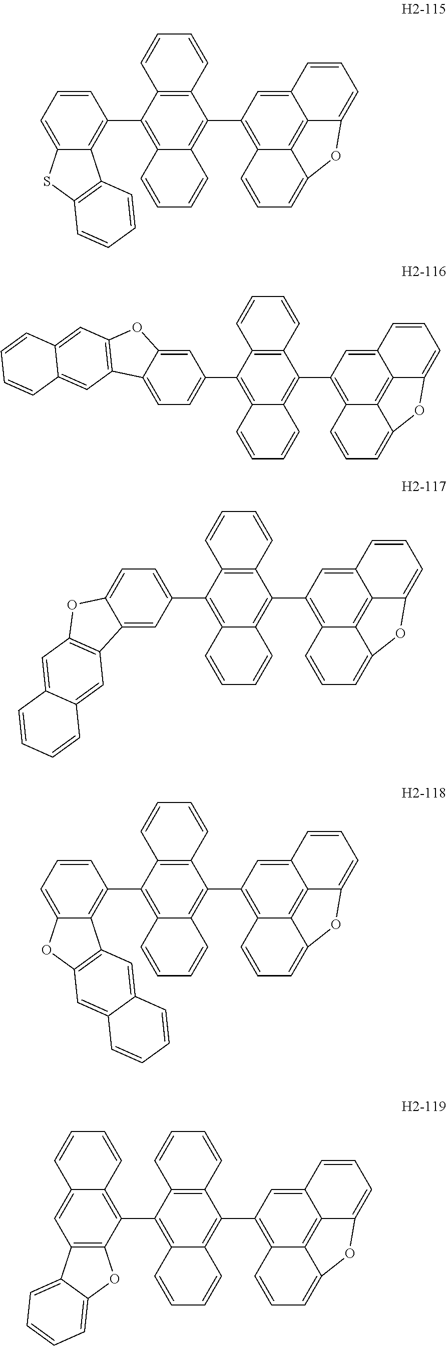 [SMILES: c1cc2cc(-c3c4ccccc4c(-c4cccc5sc6ccccc6c45)c4ccccc34)c3cccc4oc(c1)c2c43 ; c1ccc2cc3c(cc2c1)oc1cc(-c2c4ccccc4c(-c4cc5cccc6oc7cccc4c7c56)c4ccccc24)ccc13 ; c1ccc2cc3c(cc2c1)oc1ccc(-c2c4ccccc4c(-c4cc5cccc6oc7cccc4c7c56)c4ccccc24)cc13 ; c1ccc2cc3c(cc2c1)oc1cccc(-c2c4ccccc4c(-c4cc5cccc6oc7cccc4c7c56)c4ccccc24)c13 ; c1ccc2c(-c3c4ccccc4c(-c4cc5cccc6oc7cccc4c7c56)c4ccccc34)c3oc4ccccc4c3cc2c1]

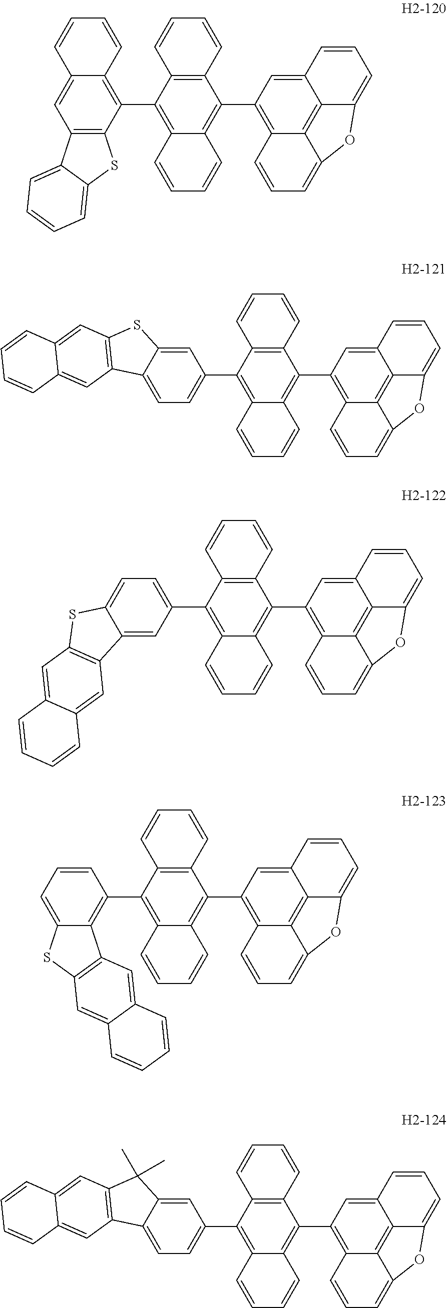 [SMILES: c1ccc2c(-c3c4ccccc4c(-c4cc5cccc6oc7cccc4c7c56)c4ccccc34)c3sc4ccccc4c3cc2c1 ; c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13 ; CC1(C)c2cc(-c3c4ccccc4c(-c4cc5cccc6oc7cccc4c7c56)c4ccccc34)ccc2-c2cc3ccccc3cc21]

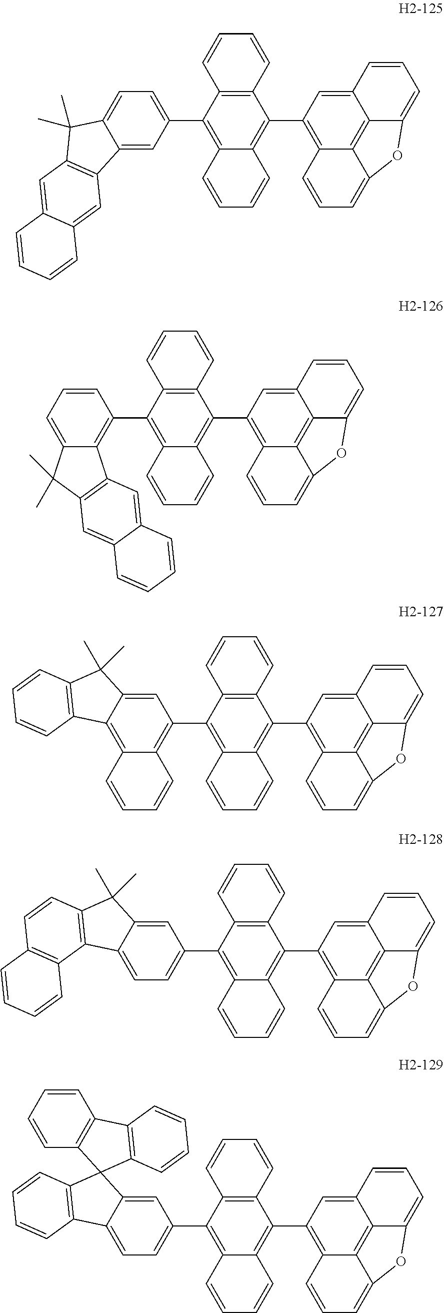 [SMILES: CC1(C)c2ccc(-c3c4ccccc4c(-c4cc5cccc6oc7cccc4c7c56)c4ccccc34)cc2-c2cc3ccccc3cc21 ; CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6oc7cccc4c7c56)c4ccccc34)cc21]

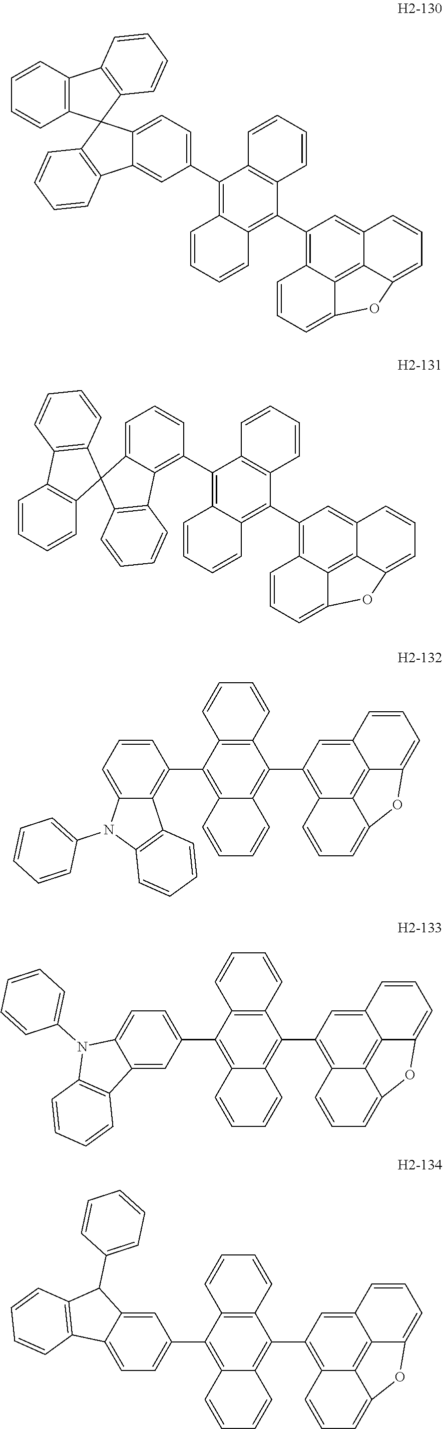 [SMILES: c1ccc2c(c1)-c1ccccc1C21c2ccccc2-c2cc(-c3c4ccccc4c(-c4cc5cccc6oc7cccc4c7c56)c4ccccc34)ccc21 ; c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1 ; c1ccc(-n2c3ccccc3c3cc(-c4c5ccccc5c(-c5cc6cccc7oc8cccc5c8c67)c5ccccc45)ccc32)cc1 ; c1ccc(C2c3ccccc3-c3ccc(-c4c5ccccc5c(-c5cc6cccc7oc8cccc5c8c67)c5ccccc45)cc32)cc1]

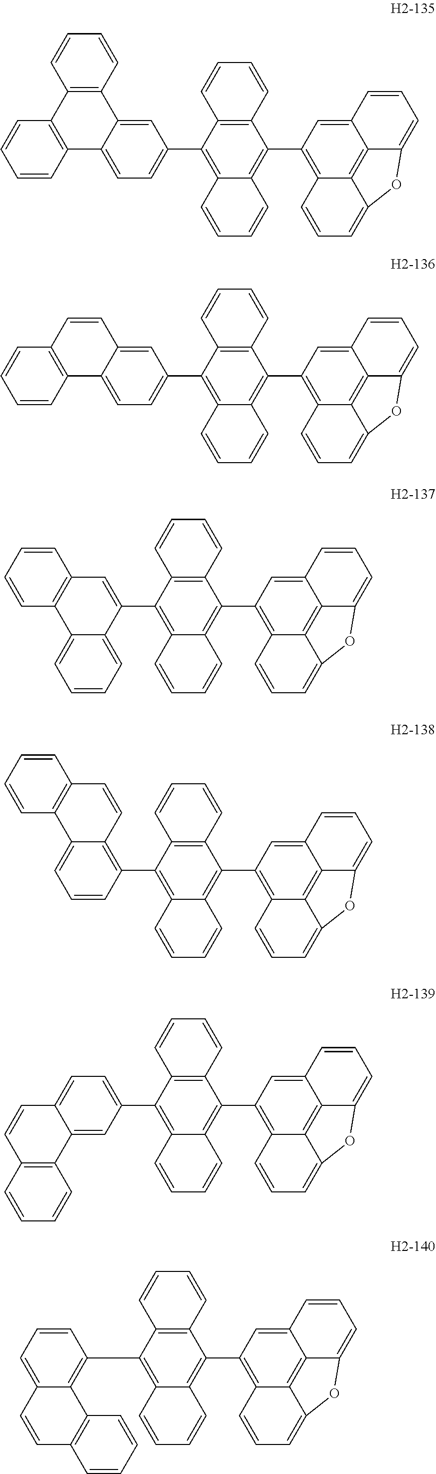 [SMILES: c1cc2cc(-c3c4ccccc4c(-c4ccc5c6ccccc6c6ccccc6c5c4)c4ccccc34)c3cccc4oc(c1)c2c43 ; c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; c1ccc2c(c1)ccc1c(-c3c4ccccc4c(-c4cc5cccc6oc7cccc4c7c56)c4ccccc34)cccc12 ; c1ccc2c(c1)ccc1ccc(-c3c4ccccc4c(-c4cc5cccc6oc7cccc4c7c56)c4ccccc34)cc12 ; c1ccc2c(c1)ccc1cccc(-c3c4ccccc4c(-c4cc5cccc6oc7cccc4c7c56)c4ccccc34)c12]

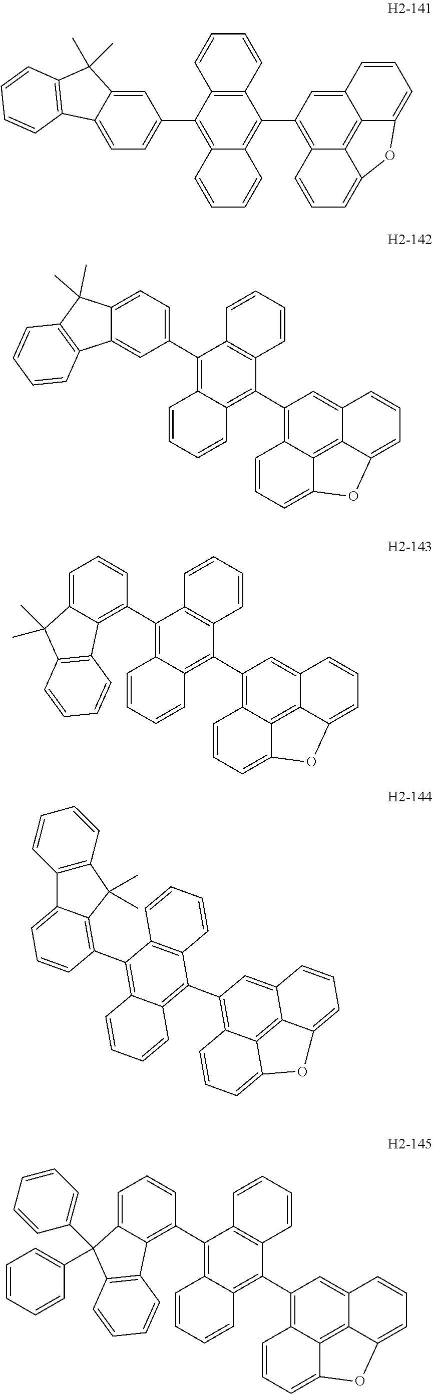 [SMILES: CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21 ; CC1(C)c2ccccc2-c2cccc(-c3c4ccccc4c(-c4cc5cccc6oc7cccc4c7c56)c4ccccc34)c21 ; c1ccc(C2(c3ccccc3)c3ccccc3-c3c(-c4c5ccccc5c(-c5cc6cccc7oc8cccc5c8c67)c5ccccc45)cccc32)cc1]

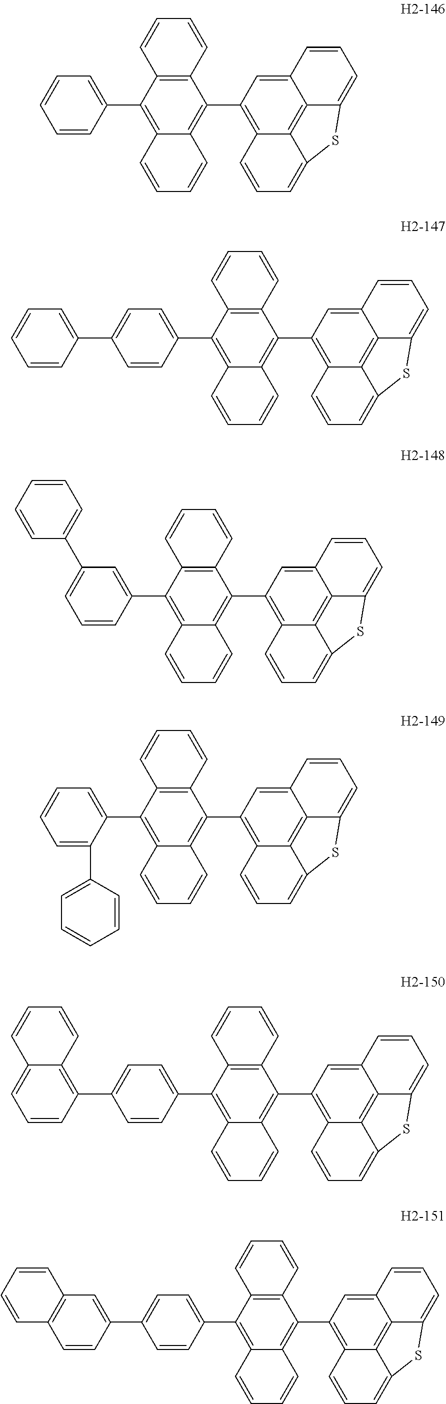 [SMILES: c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc(-c2ccccc2-c2c3ccccc3c(-c3cc4cccc5sc6cccc3c6c45)c3ccccc23)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1]

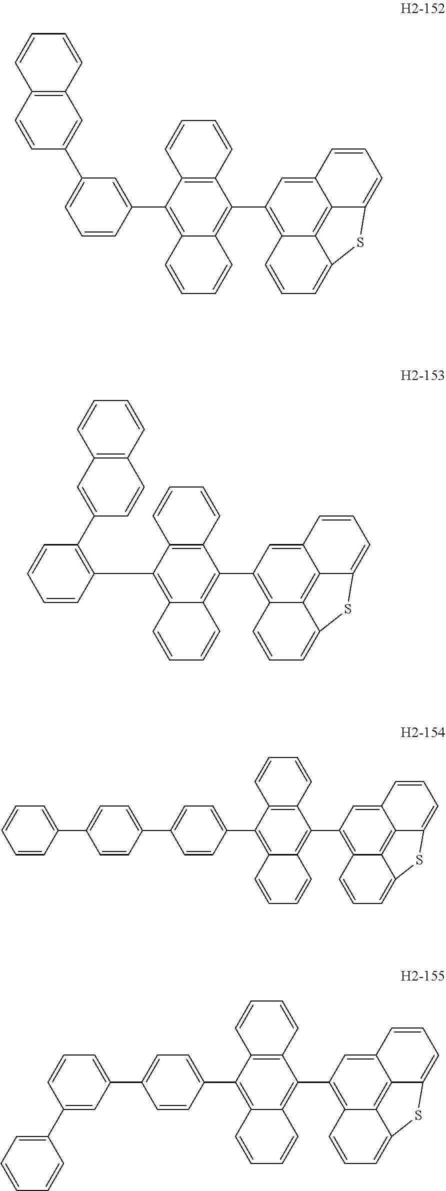 [SMILES: c1cc(-c2ccc3ccccc3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3ccccc3c2)c1 ; c1ccc(-c2ccc(-c3ccc(-c4c5ccccc5c(-c5cc6cccc7sc8cccc5c8c67)c5ccccc45)cc3)cc2)cc1 ; c1ccc(-c2cccc(-c3ccc(-c4c5ccccc5c(-c5cc6cccc7sc8cccc5c8c67)c5ccccc45)cc3)c2)cc1]

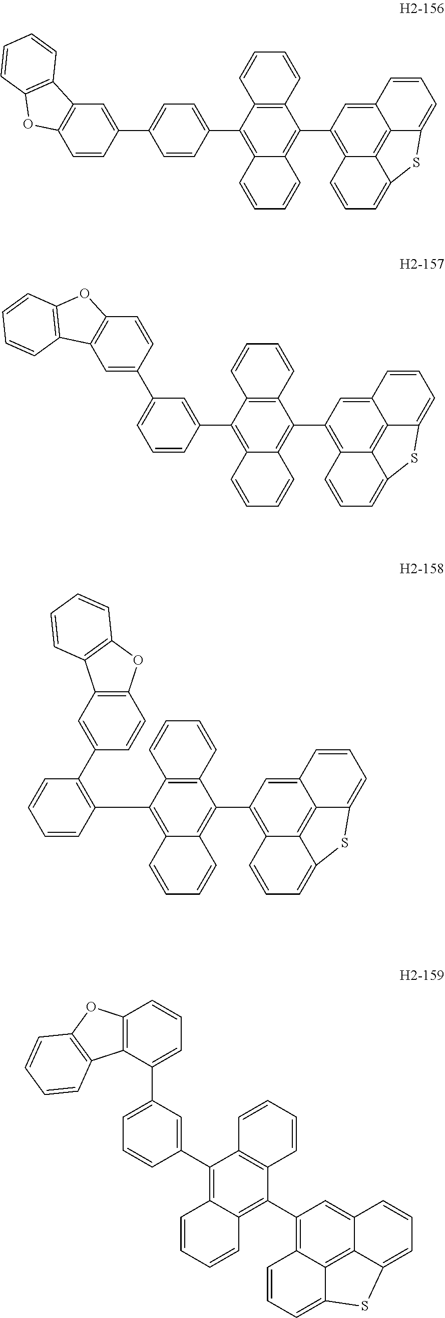 [SMILES: c1ccc2c(c1)oc1ccc(-c3ccc(-c4c5ccccc5c(-c5cc6cccc7sc8cccc5c8c67)c5ccccc45)cc3)cc12 ; c1cc(-c2ccc3oc4ccccc4c3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3oc4ccccc4c3c2)c1 ; c1cc(-c2c3ccccc3c(-c3cc4cccc5sc6cccc3c6c45)c3ccccc23)cc(-c2cccc3oc4ccccc4c23)c1]

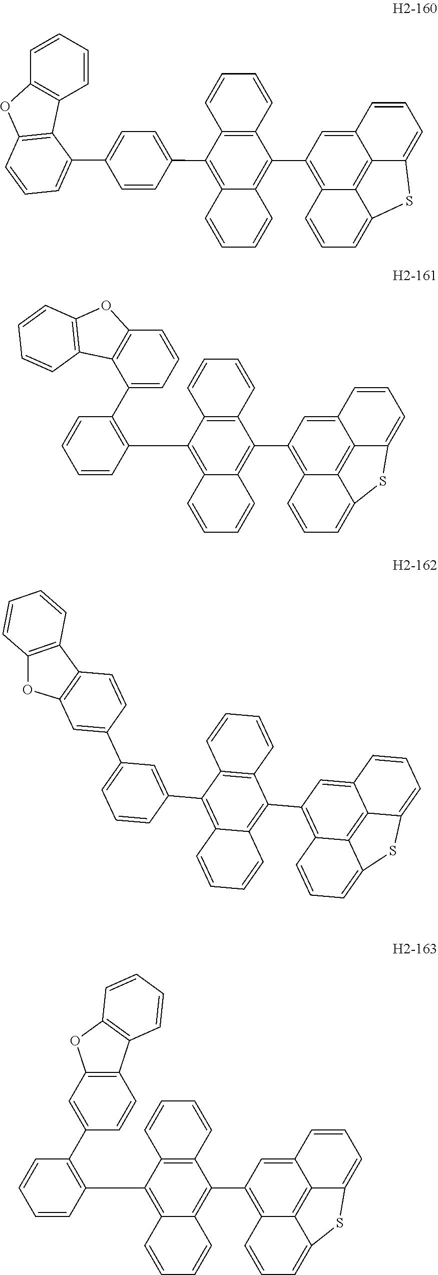 [SMILES: c1ccc2c(c1)oc1cccc(-c3ccc(-c4c5ccccc5c(-c5cc6cccc7sc8cccc5c8c67)c5ccccc45)cc3)c12 ; c1ccc(-c2cccc3oc4ccccc4c23)c(-c2c3ccccc3c(-c3cc4cccc5sc6cccc3c6c45)c3ccccc23)c1 ; c1cc(-c2ccc3c(c2)oc2ccccc23)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3c(c2)oc2ccccc23)c1]

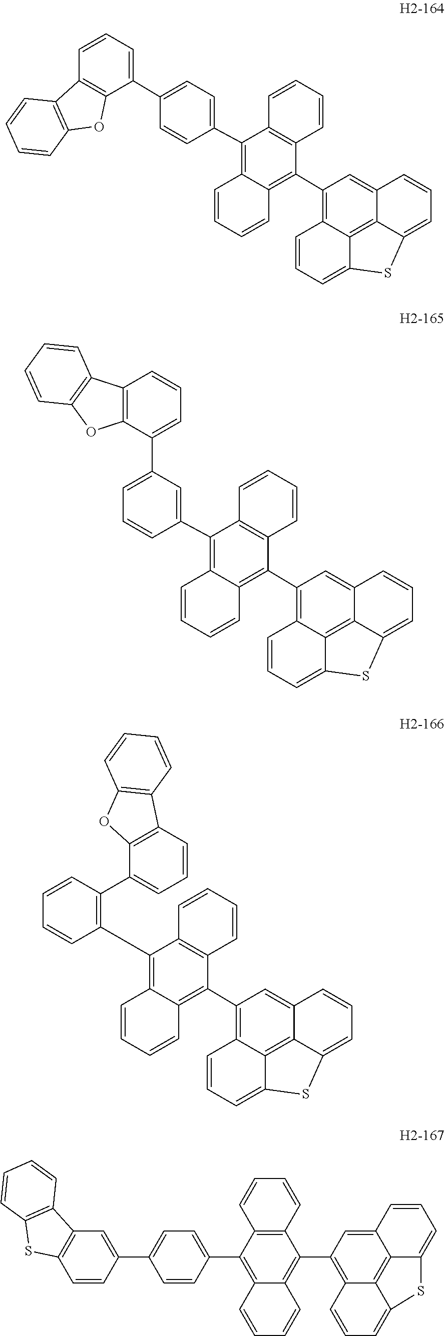 [SMILES: c1ccc2c(c1)oc1c(-c3ccc(-c4c5ccccc5c(-c5cc6cccc7sc8cccc5c8c67)c5ccccc45)cc3)cccc12 ; c1cc(-c2c3ccccc3c(-c3cc4cccc5sc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5sc6cccc3c6c45)c3ccccc23)c1 ; c1ccc2c(c1)sc1ccc(-c3ccc(-c4c5ccccc5c(-c5cc6cccc7sc8cccc5c8c67)c5ccccc45)cc3)cc12]

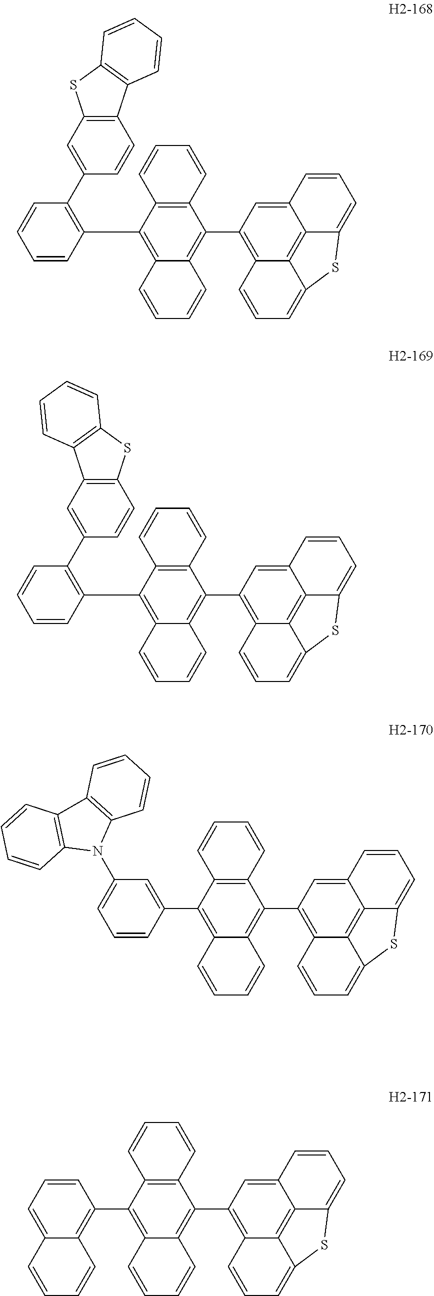 [SMILES: c1ccc(-c2c3ccccc3c(-c3cc4cccc5sc6cccc3c6c45)c3ccccc23)c(-c2ccc3c(c2)sc2ccccc23)c1 ; c1ccc(-c2c3ccccc3c(-c3cc4cccc5sc6cccc3c6c45)c3ccccc23)c(-c2ccc3sc4ccccc4c3c2)c1 ; c1cc(-c2c3ccccc3c(-c3cc4cccc5sc6cccc3c6c45)c3ccccc23)cc(-n2c3ccccc3c3ccccc32)c1 ; c1ccc2c(-c3c4ccccc4c(-c4cc5cccc6sc7cccc4c7c56)c4ccccc34)cccc2c1]

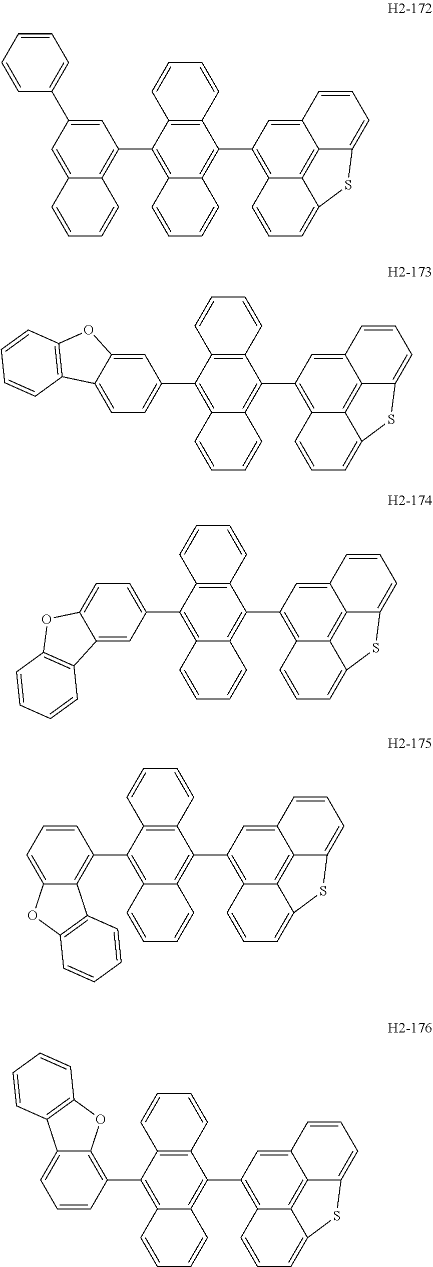 [SMILES: c1ccc(-c2cc(-c3c4ccccc4c(-c4cc5cccc6sc7cccc4c7c56)c4ccccc34)c3ccccc3c2)cc1 ; c1ccc2c(c1)oc1cc(-c3c4ccccc4c(-c4cc5cccc6sc7cccc4c7c56)c4ccccc34)ccc12 ; c1ccc2c(c1)oc1ccc(-c3c4ccccc4c(-c4cc5cccc6sc7cccc4c7c56)c4ccccc34)cc12 ; c1ccc2c(c1)oc1cccc(-c3c4ccccc4c(-c4cc5cccc6sc7cccc4c7c56)c4ccccc34)c12 ; c1ccc2c(c1)oc1c(-c3c4ccccc4c(-c4cc5cccc6sc7cccc4c7c56)c4ccccc34)cccc12]

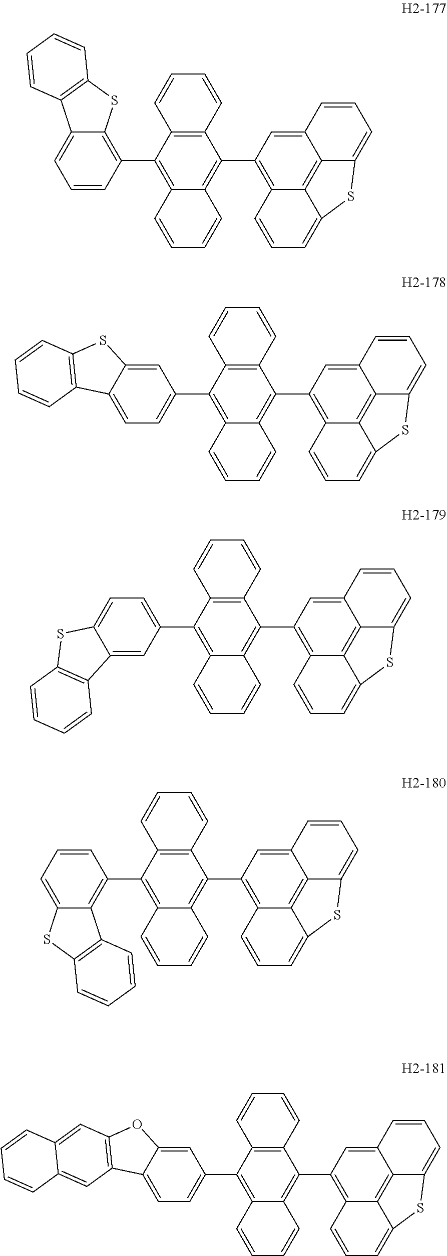 [SMILES: c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; c1ccc2cc3c(cc2c1)oc1cc(-c2c4ccccc4c(-c4cc5cccc6sc7cccc4c7c56)c4ccccc24)ccc13]

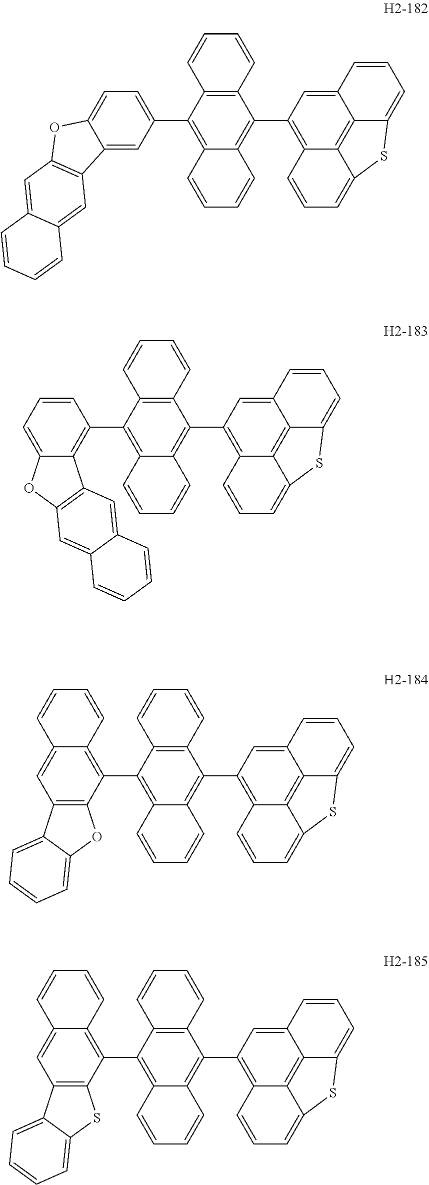 [SMILES: c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1 ; c1ccc2c(-c3c4ccccc4c(-c4cc5cccc6sc7cccc4c7c56)c4ccccc34)c3sc4ccccc4c3cc2c1]

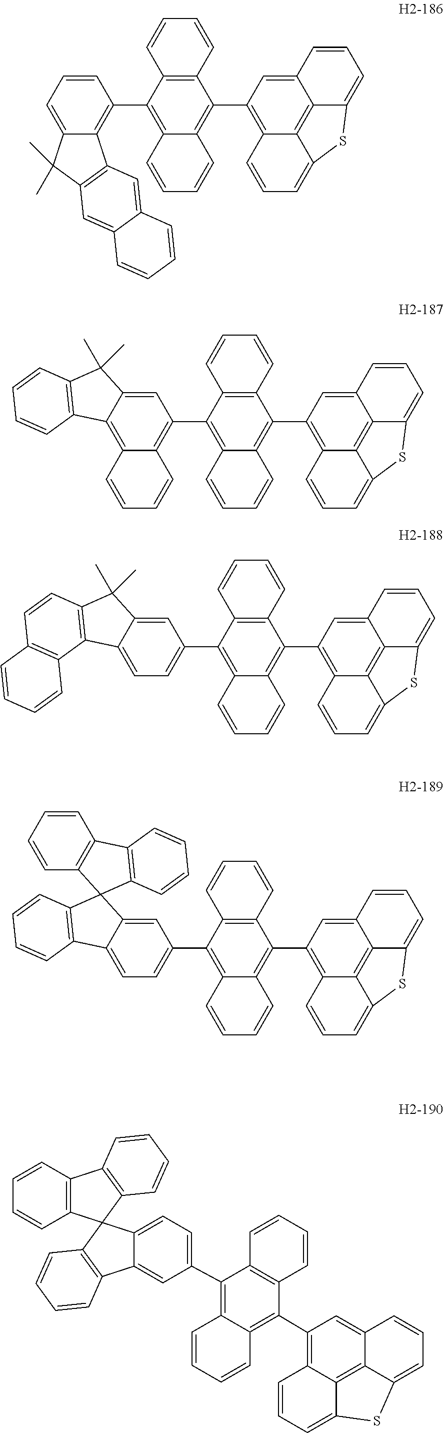 [SMILES: CC1(C)c2cc3ccccc3cc2-c2c(-c3c4ccccc4c(-c4cc5cccc6sc7cccc4c7c56)c4ccccc34)cccc21 ; CC1(C)c2ccccc2-c2c1cc(-c1c3ccccc3c(-c3cc4cccc5sc6cccc3c6c45)c3ccccc13)c1ccccc21 ; CC1(C)c2cc(-c3c4ccccc4c(-c4cc5cccc6sc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6sc7cccc4c7c56)c4ccccc34)cc21 ; c1ccc2c(c1)-c1ccccc1C21c2ccccc2-c2cc(-c3c4ccccc4c(-c4cc5cccc6sc7cccc4c7c56)c4ccccc34)ccc21]

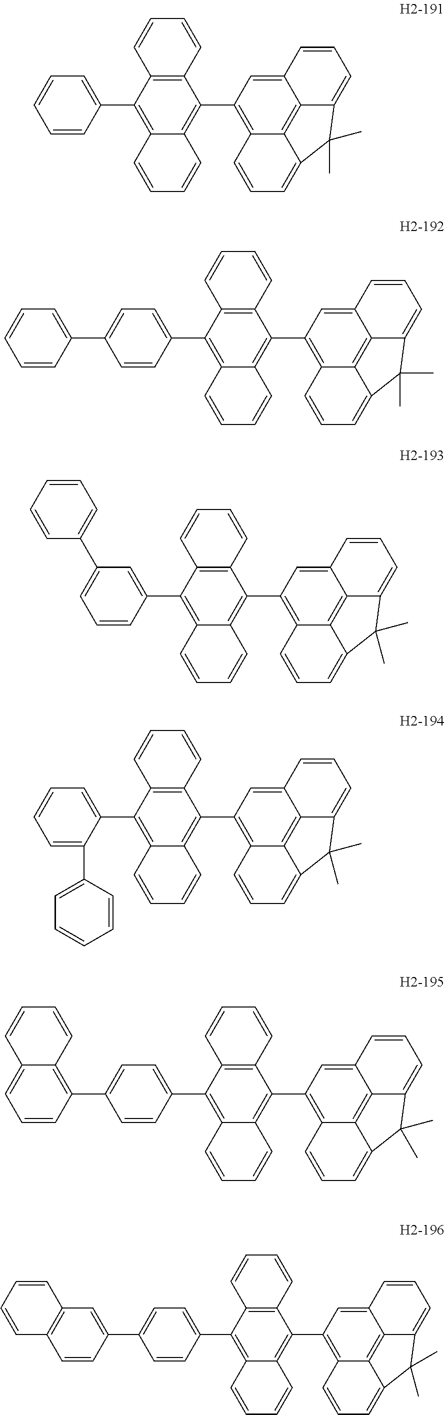

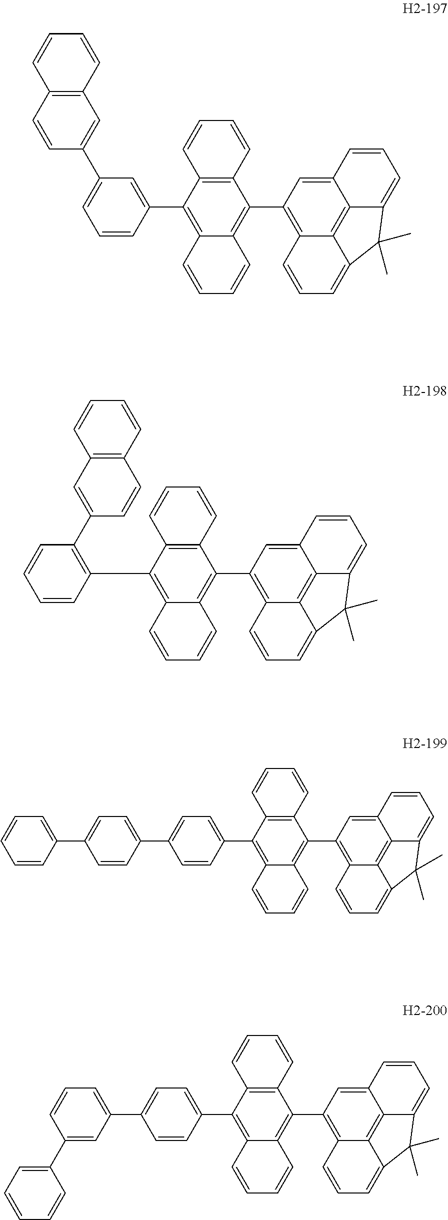

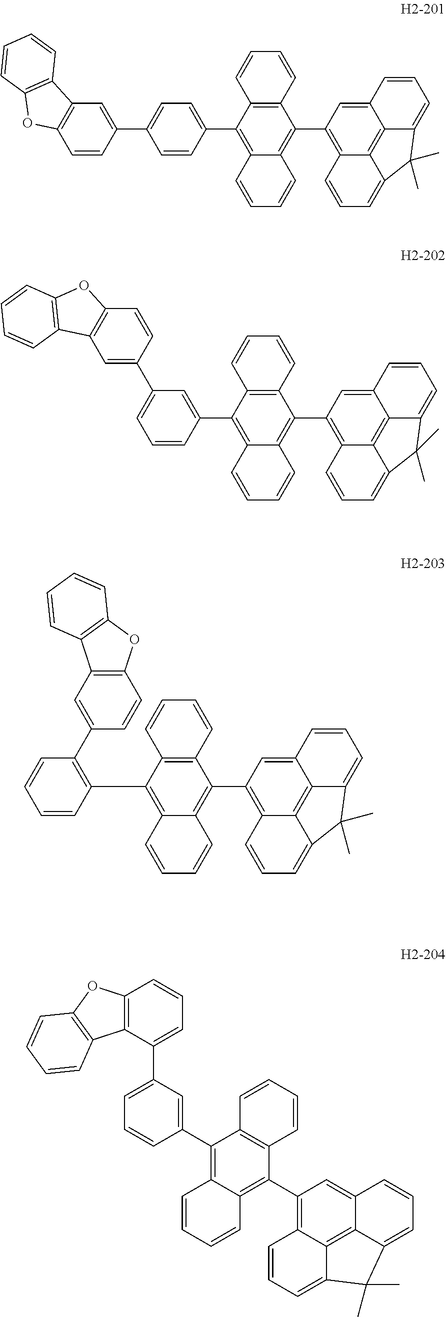 [SMILES: CC1(C)c2cccc3cc(-c4c5ccccc5c(-c5ccc(-c6ccc7oc8ccccc8c7c6)cc5)c5ccccc45)c4cccc1c4c23 ; CC1(C)c2cccc3cc(-c4c5ccccc5c(-c5cccc(-c6ccc7oc8ccccc8c7c6)c5)c5ccccc45)c4cccc1c4c23 ; CC1(C)c2cccc3cc(-c4c5ccccc5c(-c5ccccc5-c5ccc6oc7ccccc7c6c5)c5ccccc45)c4cccc1c4c23 ; CC1(C)c2cccc3cc(-c4c5ccccc5c(-c5cccc(-c6cccc7oc8ccccc8c67)c5)c5ccccc45)c4cccc1c4c23]

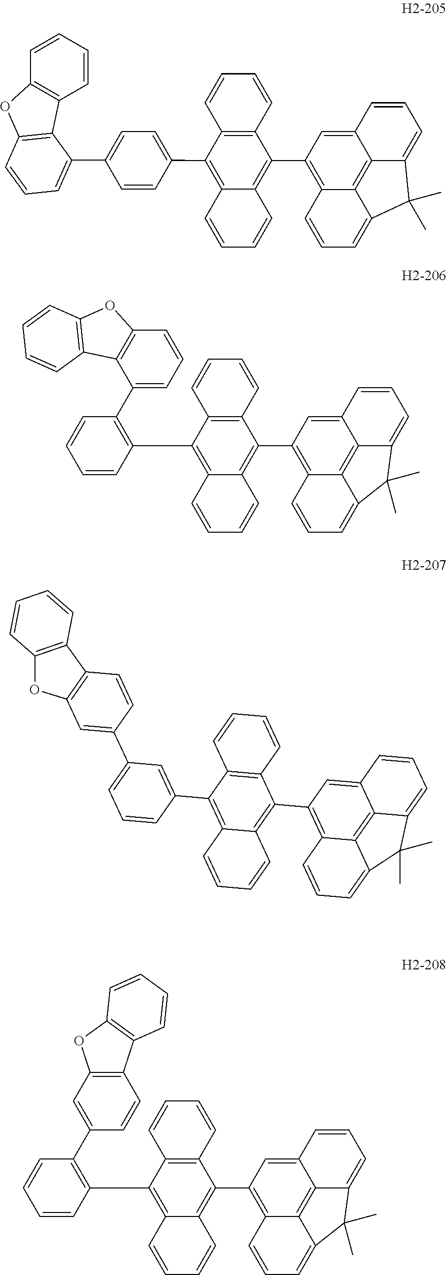 [SMILES: CC1(C)c2cccc3cc(-c4c5ccccc5c(-c5ccc(-c6cccc7oc8ccccc8c67)cc5)c5ccccc45)c4cccc1c4c23 ; CC1(C)c2cccc3cc(-c4c5ccccc5c(-c5ccccc5-c5cccc6oc7ccccc7c56)c5ccccc45)c4cccc1c4c23 ; CC1(C)c2cccc3cc(-c4c5ccccc5c(-c5cccc(-c6ccc7c(c6)oc6ccccc67)c5)c5ccccc45)c4cccc1c4c23 ; CC1(C)c2cccc3cc(-c4c5ccccc5c(-c5ccccc5-c5ccc6c(c5)oc5ccccc56)c5ccccc45)c4cccc1c4c23]

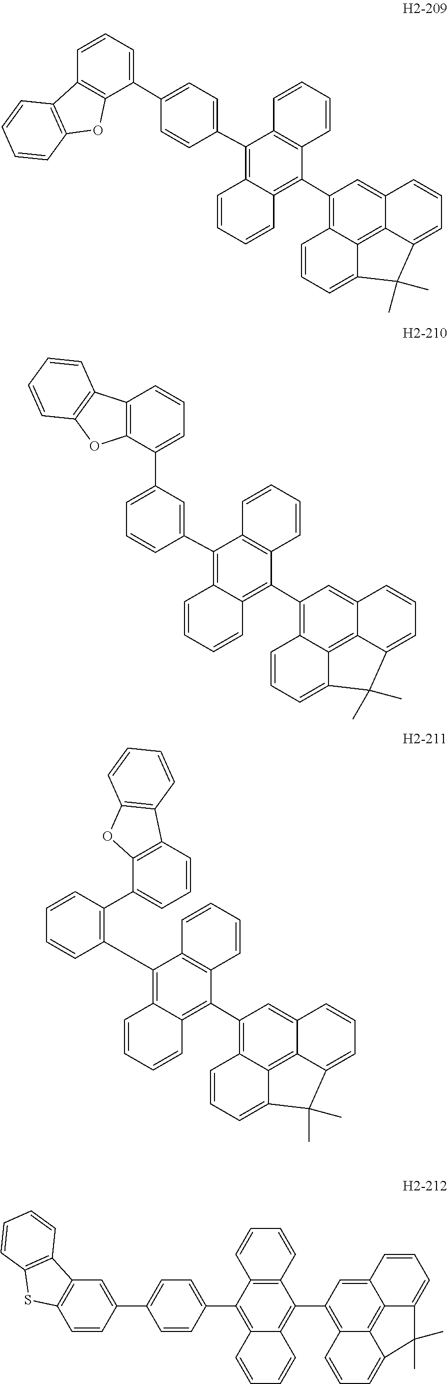 [SMILES: CC1(C)c2cccc3cc(-c4c5ccccc5c(-c5ccc(-c6cccc7c6oc6ccccc67)cc5)c5ccccc45)c4cccc1c4c23 ; CC1(C)c2cccc3cc(-c4c5ccccc5c(-c5cccc(-c6cccc7c6oc6ccccc67)c5)c5ccccc45)c4cccc1c4c23 ; CC1(C)c2cccc3cc(-c4c5ccccc5c(-c5ccccc5-c5cccc6c5oc5ccccc56)c5ccccc45)c4cccc1c4c23 ; CC1(C)c2cccc3cc(-c4c5ccccc5c(-c5ccc(-c6ccc7sc8ccccc8c7c6)cc5)c5ccccc45)c4cccc1c4c23]

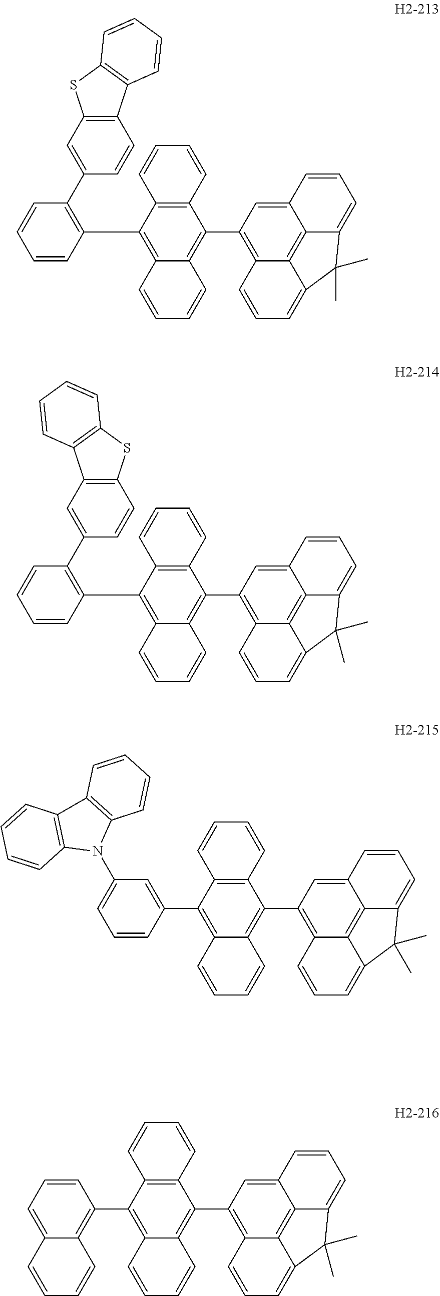

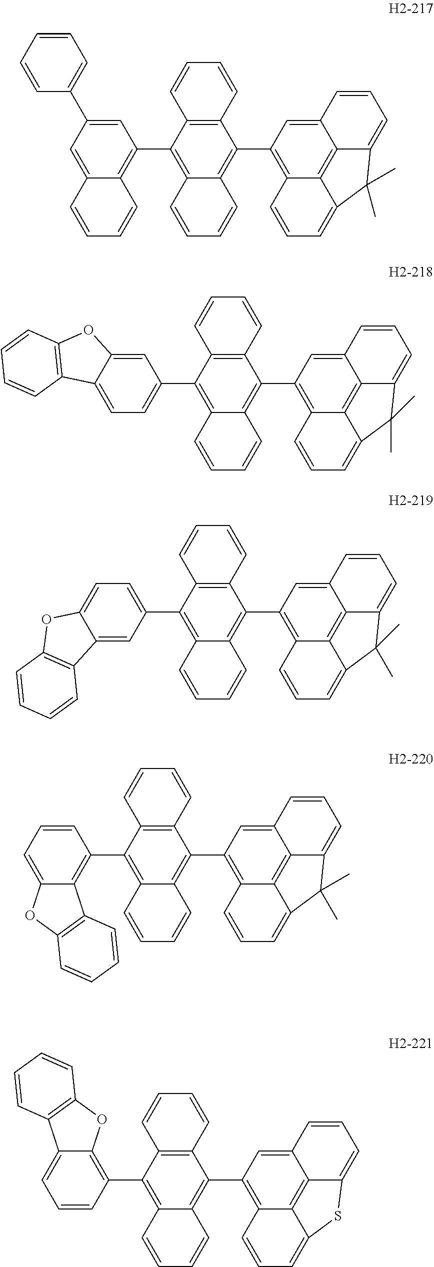

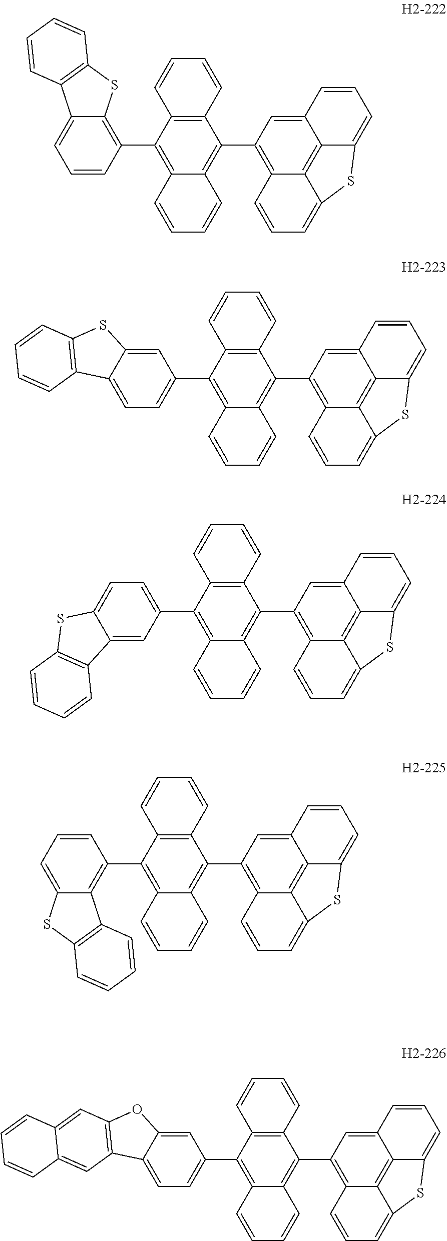 [SMILES: c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; c1ccc2cc3c(cc2c1)oc1cc(-c2c4ccccc4c(-c4cc5cccc6sc7cccc4c7c56)c4ccccc24)ccc13]

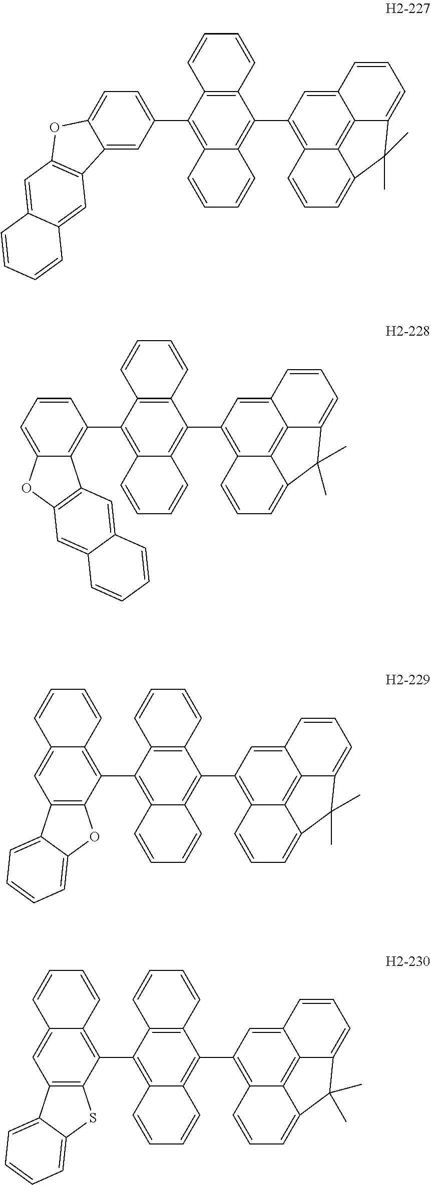 [SMILES: CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23 ; CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23]

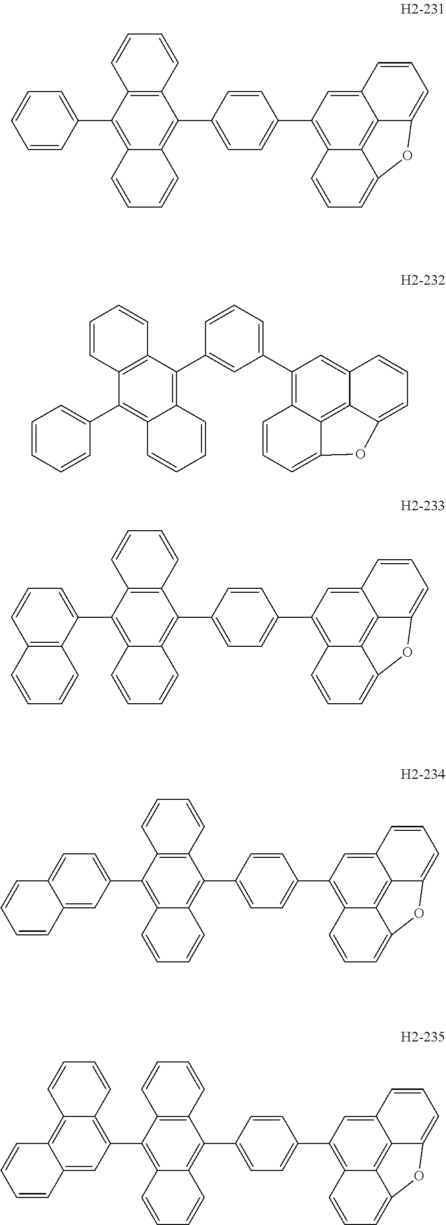 [SMILES: c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12]

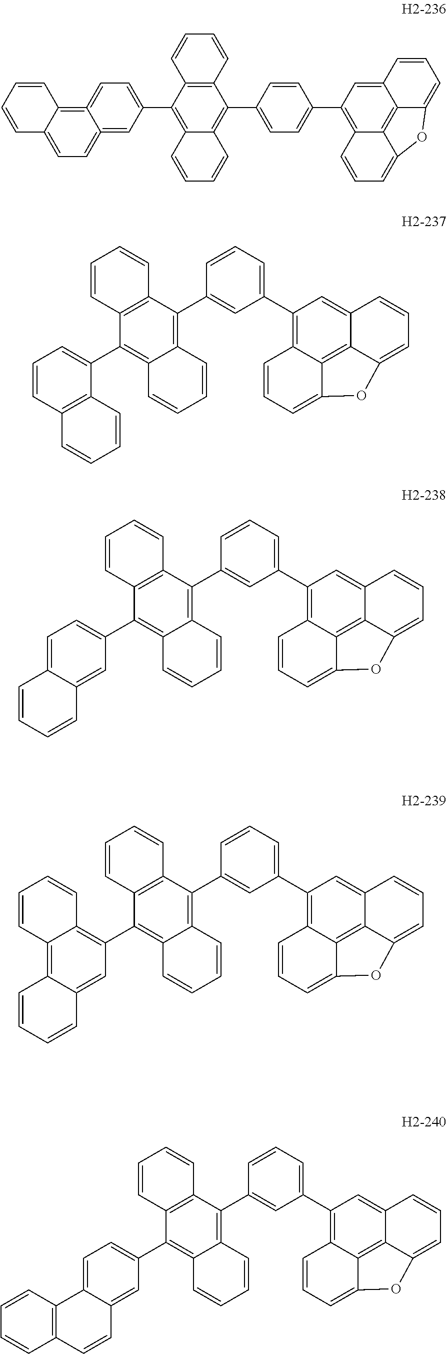

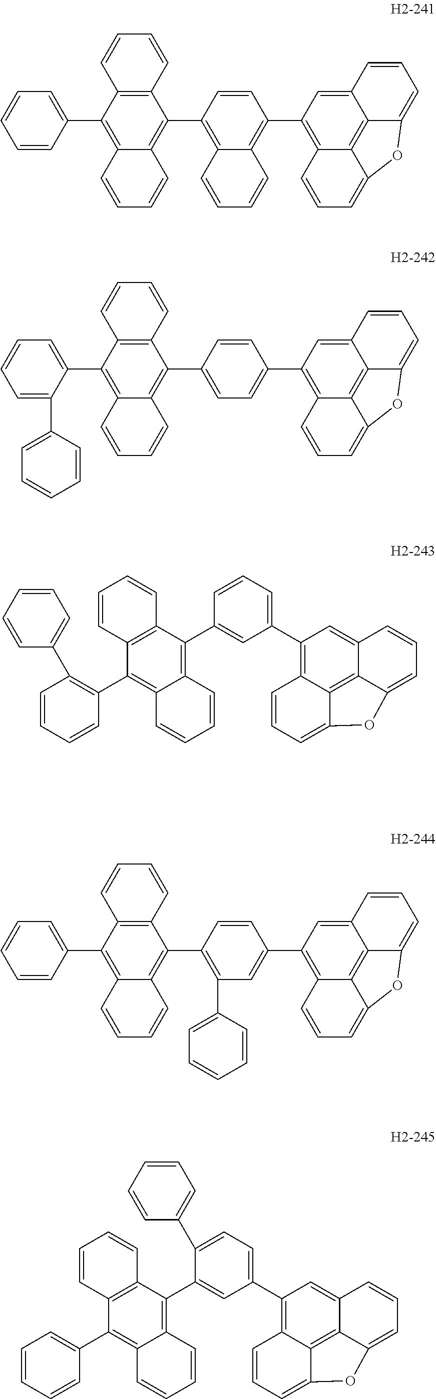 [SMILES: c1ccc(-c2c3ccccc3c(-c3ccc(-c4cc5cccc6oc7cccc4c7c56)c4ccccc34)c3ccccc23)cc1 ; c1ccc(-c2ccccc2-c2c3ccccc3c(-c3ccc(-c4cc5cccc6oc7cccc4c7c56)cc3)c3ccccc23)cc1 ; c1ccc(-c2ccccc2-c2c3ccccc3c(-c3cccc(-c4cc5cccc6oc7cccc4c7c56)c3)c3ccccc23)cc1 ; c1ccc(-c2cc(-c3cc4cccc5oc6cccc3c6c45)ccc2-c2c3ccccc3c(-c3ccccc3)c3ccccc23)cc1 ; c1ccc(-c2ccc(-c3cc4cccc5oc6cccc3c6c45)cc2-c2c3ccccc3c(-c3ccccc3)c3ccccc23)cc1]

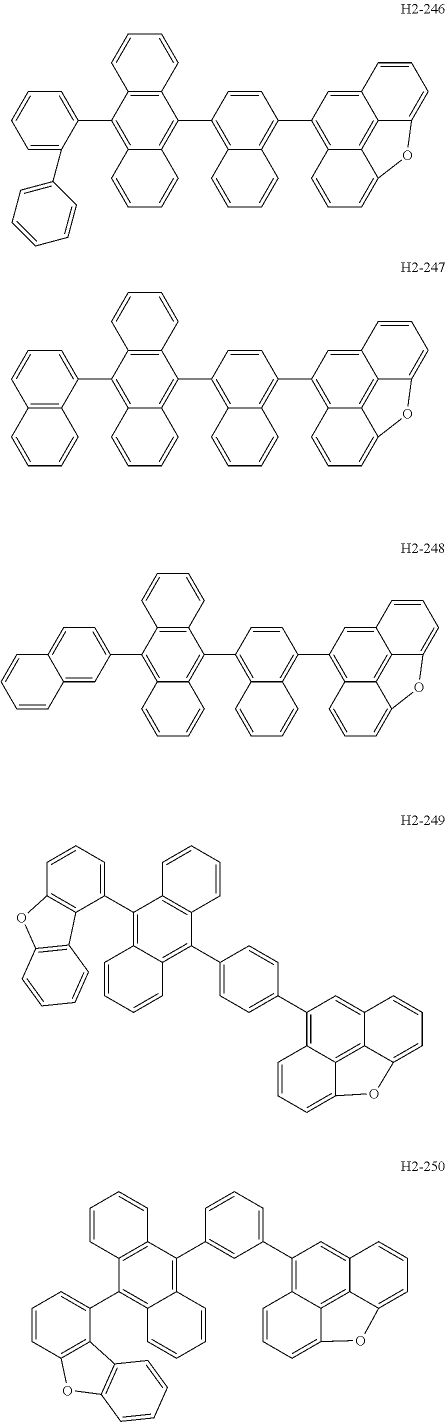 [SMILES: c1ccc(-c2ccccc2-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1]

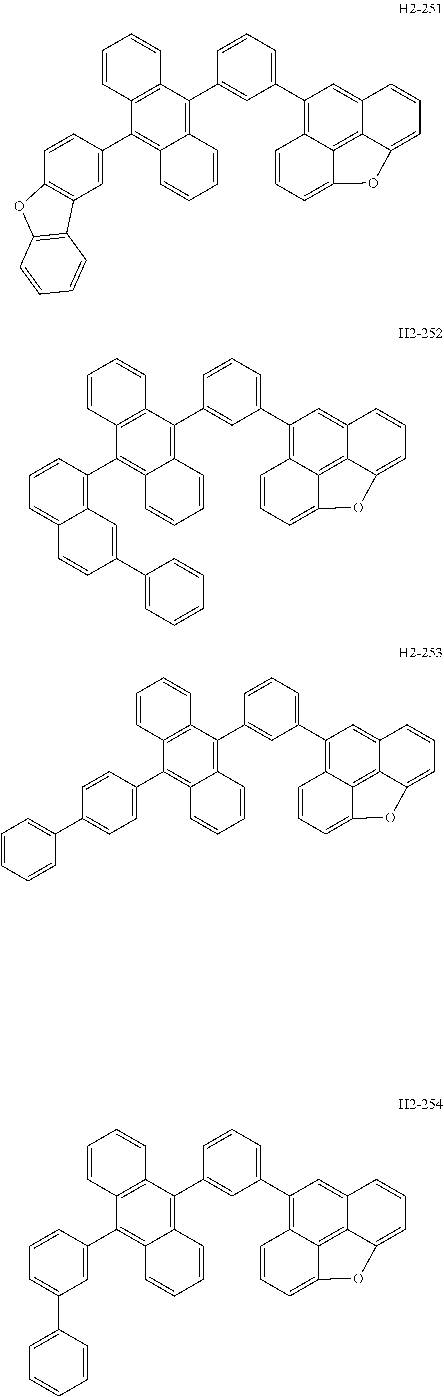 [SMILES: c1cc(-c2c3ccccc3c(-c3ccc4oc5ccccc5c4c3)c3ccccc23)cc(-c2cc3cccc4oc5cccc2c5c34)c1 ; c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1 ; c1ccc(-c2cccc(-c3c4ccccc4c(-c4cccc(-c5cc6cccc7oc8cccc5c8c67)c4)c4ccccc34)c2)cc1]

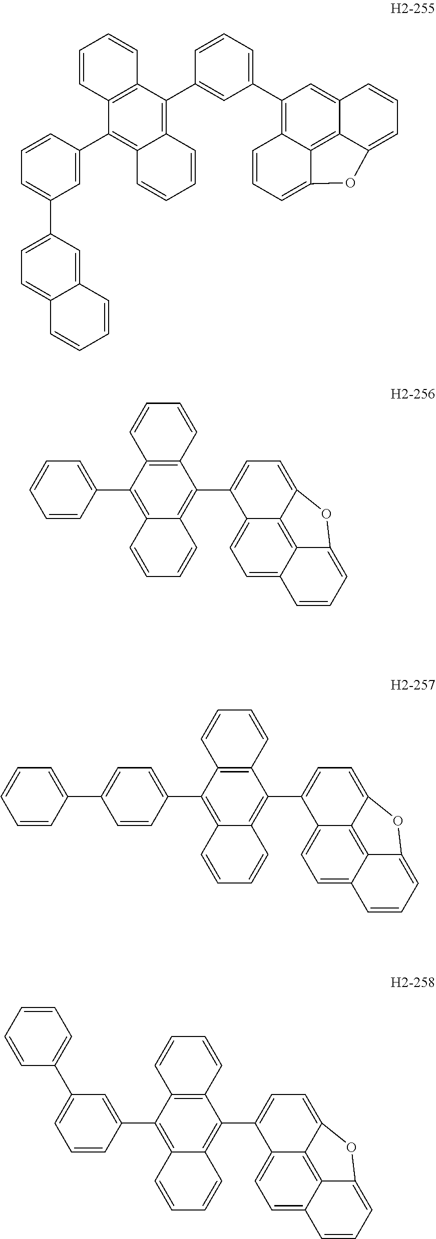 [SMILES: c1cc(-c2ccc3ccccc3c2)cc(-c2c3ccccc3c(-c3cccc(-c4cc5cccc6oc7cccc4c7c56)c3)c3ccccc23)c1 ; c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1]

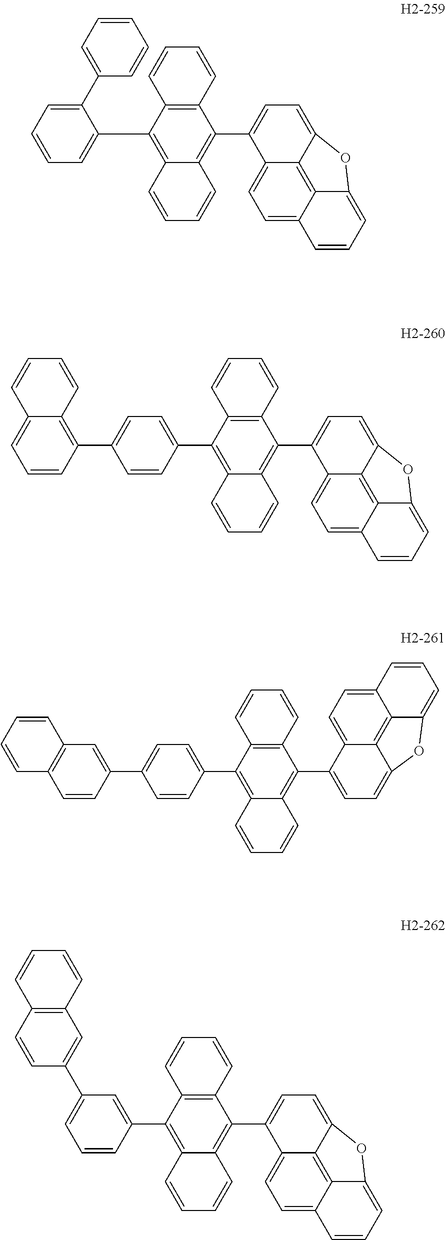 [SMILES: c1ccc(-c2ccccc2-c2c3ccccc3c(-c3ccc4oc5cccc6ccc3c4c65)c3ccccc23)cc1 ; c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1 ; c1cc(-c2ccc3ccccc3c2)cc(-c2c3ccccc3c(-c3ccc4oc5cccc6ccc3c4c65)c3ccccc23)c1]

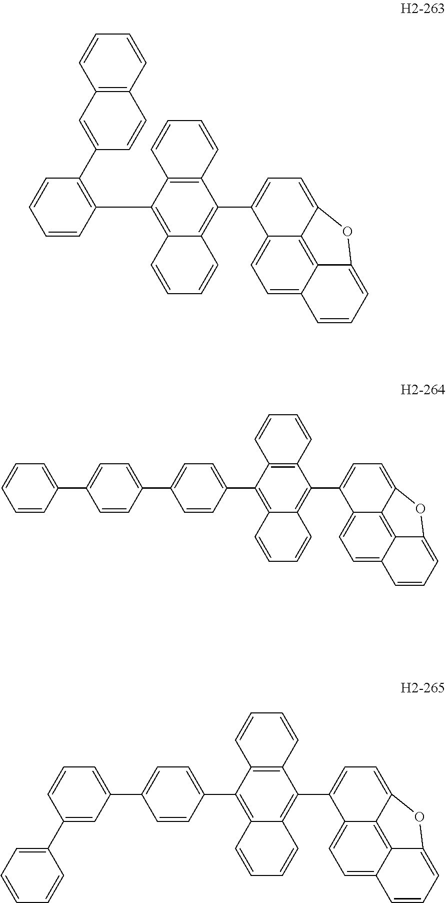 [SMILES: c1ccc(-c2c3ccccc3c(-c3ccc4oc5cccc6ccc3c4c65)c3ccccc23)c(-c2ccc3ccccc3c2)c1 ; c1ccc(-c2ccc(-c3ccc(-c4c5ccccc5c(-c5ccc6oc7cccc8ccc5c6c87)c5ccccc45)cc3)cc2)cc1 ; c1ccc(-c2cccc(-c3ccc(-c4c5ccccc5c(-c5ccc6oc7cccc8ccc5c6c87)c5ccccc45)cc3)c2)cc1]

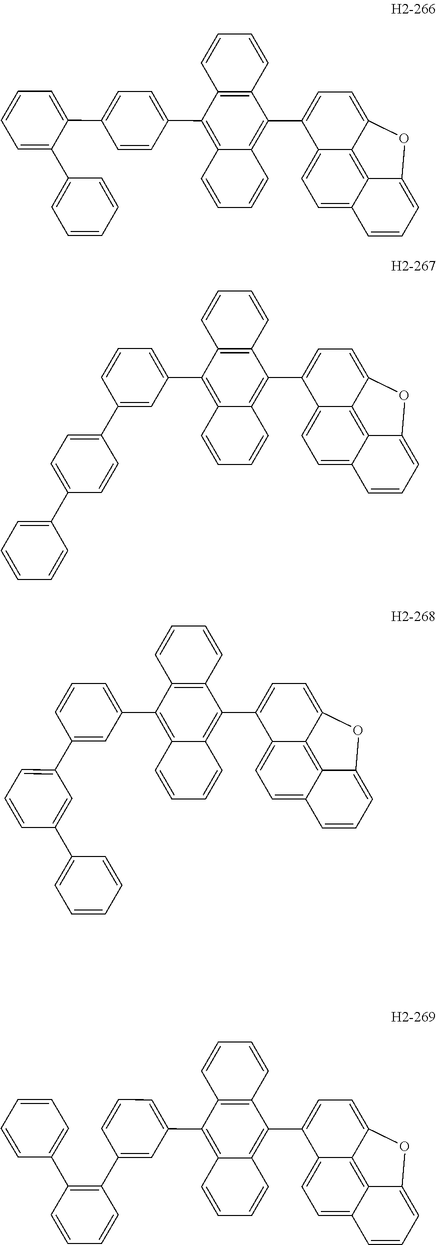 [SMILES: c1ccc(-c2ccccc2-c2ccc(-c3c4ccccc4c(-c4ccc5oc6cccc7ccc4c5c76)c4ccccc34)cc2)cc1 ; c1ccc(-c2ccc(-c3cccc(-c4c5ccccc5c(-c5ccc6oc7cccc8ccc5c6c87)c5ccccc45)c3)cc2)cc1 ; c1ccc(-c2cccc(-c3cccc(-c4c5ccccc5c(-c5ccc6oc7cccc8ccc5c6c87)c5ccccc45)c3)c2)cc1 ; c1ccc(-c2ccccc2-c2cccc(-c3c4ccccc4c(-c4ccc5oc6cccc7ccc4c5c76)c4ccccc34)c2)cc1]

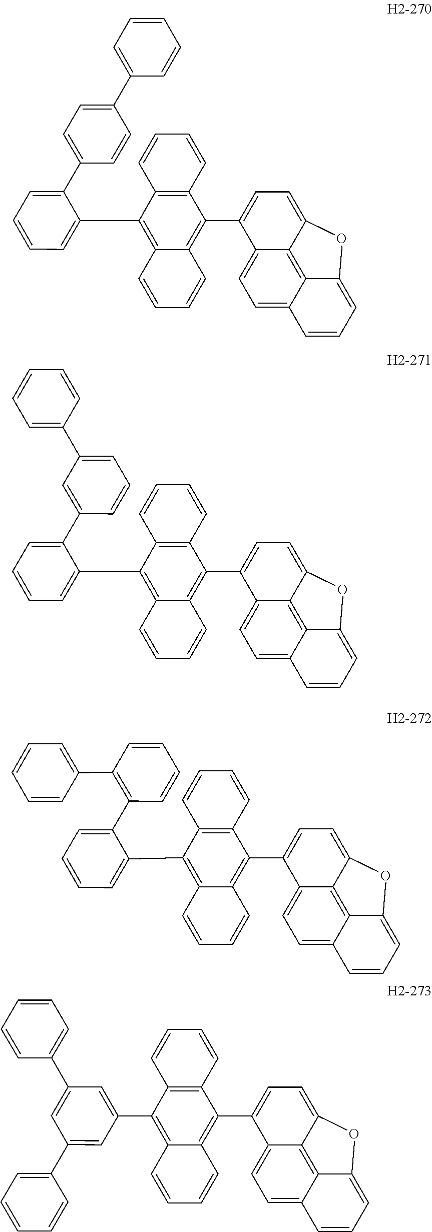 [SMILES: c1ccc(-c2ccc(-c3ccccc3-c3c4ccccc4c(-c4ccc5oc6cccc7ccc4c5c76)c4ccccc34)cc2)cc1 ; c1ccc(-c2cccc(-c3ccccc3-c3c4ccccc4c(-c4ccc5oc6cccc7ccc4c5c76)c4ccccc34)c2)cc1 ; c1ccc(-c2ccccc2-c2ccccc2-c2c3ccccc3c(-c3ccc4oc5cccc6ccc3c4c65)c3ccccc23)cc1 ; c1ccc(-c2cc(-c3ccccc3)cc(-c3c4ccccc4c(-c4ccc5oc6cccc7ccc4c5c76)c4ccccc34)c2)cc1]

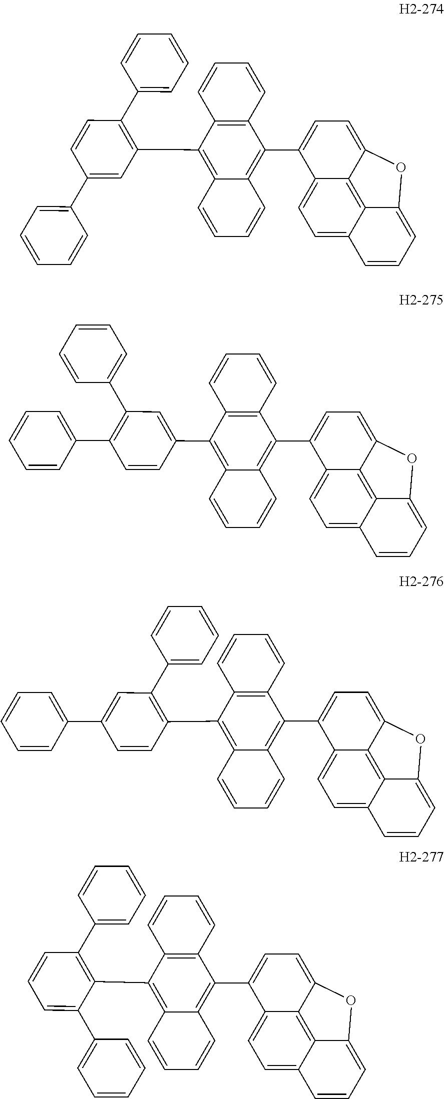 [SMILES: c1ccc(-c2ccc(-c3ccccc3)c(-c3c4ccccc4c(-c4ccc5oc6cccc7ccc4c5c76)c4ccccc34)c2)cc1 ; c1ccc(-c2ccc(-c3c4ccccc4c(-c4ccc5oc6cccc7ccc4c5c76)c4ccccc34)cc2-c2ccccc2)cc1 ; c1ccc(-c2ccc(-c3c4ccccc4c(-c4ccc5oc6cccc7ccc4c5c76)c4ccccc34)c(-c3ccccc3)c2)cc1 ; c1ccc(-c2cccc(-c3ccccc3)c2-c2c3ccccc3c(-c3ccc4oc5cccc6ccc3c4c65)c3ccccc23)cc1]

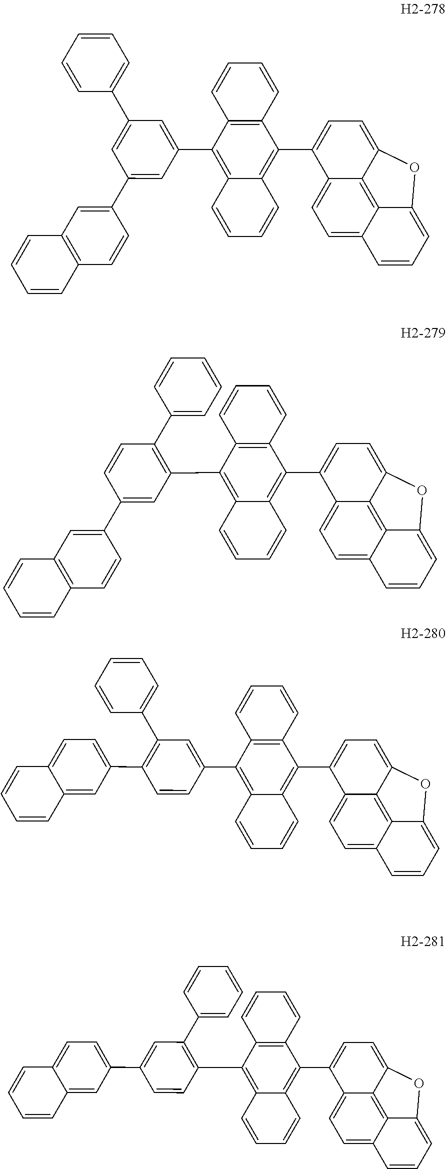 [SMILES: c1ccc(-c2cc(-c3ccc4ccccc4c3)cc(-c3c4ccccc4c(-c4ccc5oc6cccc7ccc4c5c76)c4ccccc34)c2)cc1 ; c1ccc(-c2ccc(-c3ccc4ccccc4c3)cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)ccc2-c2ccc3ccccc3c2)cc1 ; c1ccc(-c2cc(-c3ccc4ccccc4c3)ccc2-c2c3ccccc3c(-c3ccc4oc5cccc6ccc3c4c65)c3ccccc23)cc1]

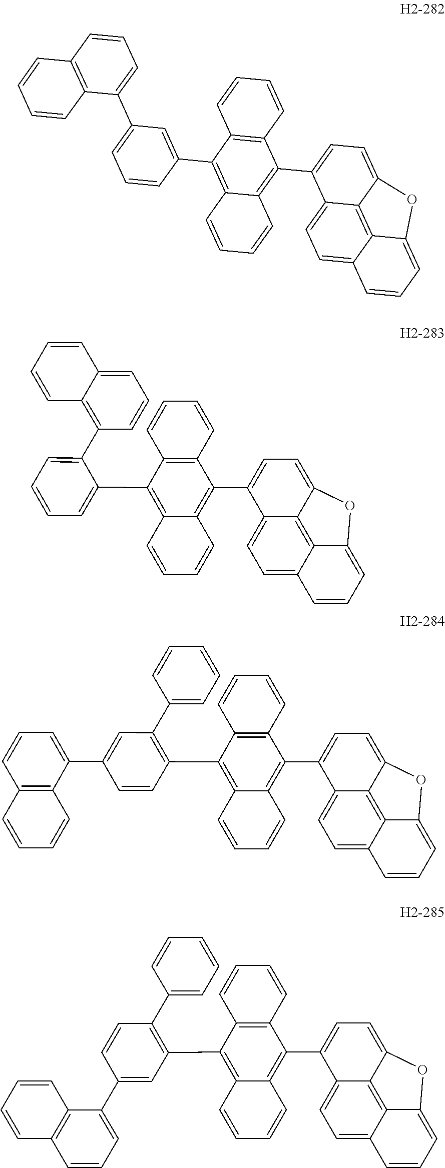 [SMILES: c1cc(-c2cccc3ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2cccc3ccccc23)c1 ; c1ccc(-c2cc(-c3cccc4ccccc34)ccc2-c2c3ccccc3c(-c3ccc4oc5cccc6ccc3c4c65)c3ccccc23)cc1 ; c1ccc(-c2ccc(-c3cccc4ccccc34)cc2-c2c3ccccc3c(-c3ccc4oc5cccc6ccc3c4c65)c3ccccc23)cc1]

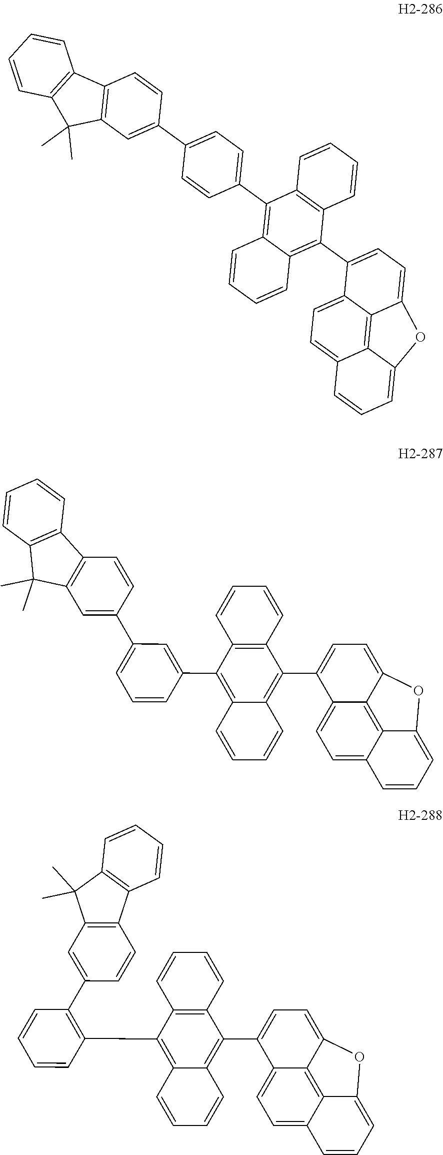 [SMILES: CC1(C)c2ccccc2-c2ccc(-c3ccc(-c4c5ccccc5c(-c5ccc6oc7cccc8ccc5c6c87)c5ccccc45)cc3)cc21 ; CC1(C)c2ccccc2-c2ccc(-c3cccc(-c4c5ccccc5c(-c5ccc6oc7cccc8ccc5c6c87)c5ccccc45)c3)cc21 ; CC1(C)c2ccccc2-c2ccc(-c3ccccc3-c3c4ccccc4c(-c4ccc5oc6cccc7ccc4c5c76)c4ccccc34)cc21]

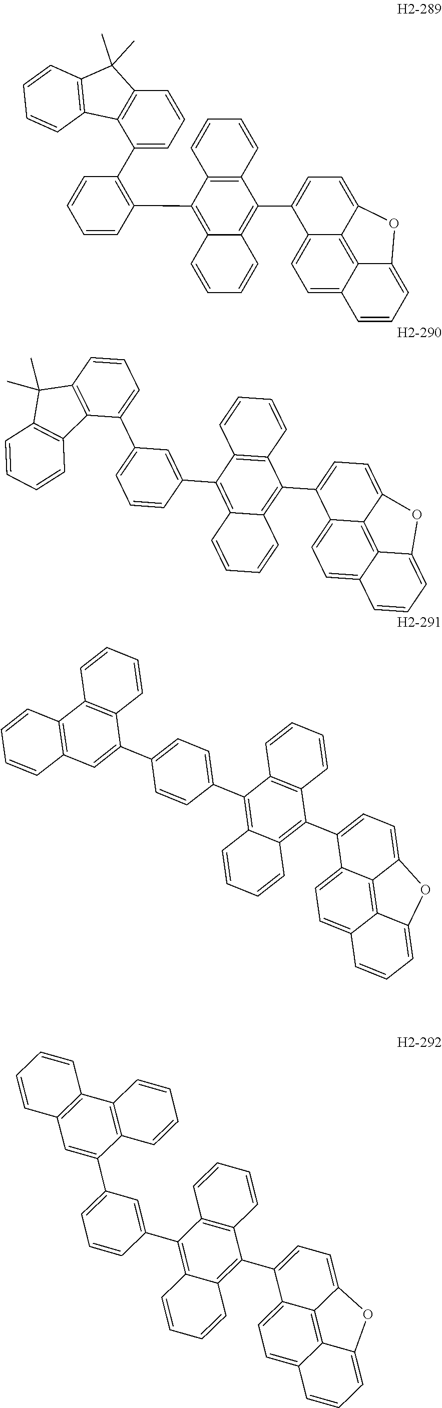 [SMILES: CC1(C)c2ccccc2-c2c(-c3ccccc3-c3c4ccccc4c(-c4ccc5oc6cccc7ccc4c5c76)c4ccccc34)cccc21 ; CC1(C)c2ccccc2-c2c(-c3cccc(-c4c5ccccc5c(-c5ccc6oc7cccc8ccc5c6c87)c5ccccc45)c3)cccc21 ; c1ccc2c(c1)cc(-c1ccc(-c3c4ccccc4c(-c4ccc5oc6cccc7ccc4c5c76)c4ccccc34)cc1)c1ccccc12 ; c1cc(-c2c3ccccc3c(-c3ccc4oc5cccc6ccc3c4c65)c3ccccc23)cc(-c2cc3ccccc3c3ccccc23)c1]

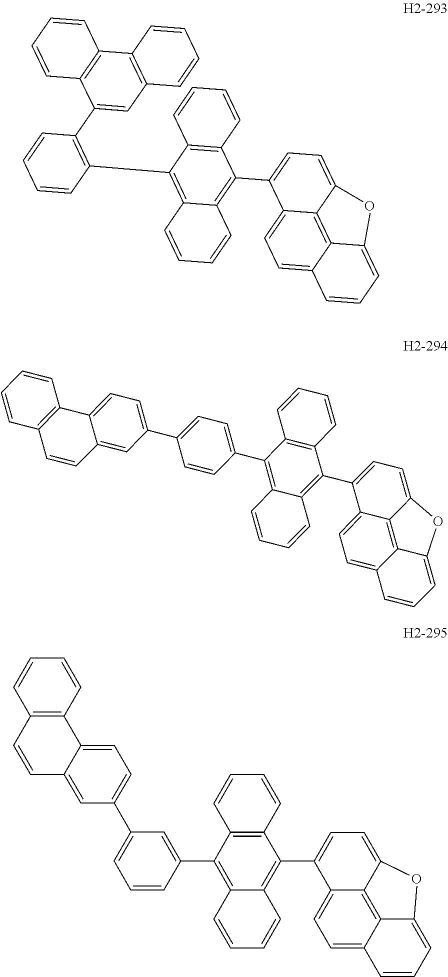 [SMILES: c1ccc(-c2cc3ccccc3c3ccccc23)c(-c2c3ccccc3c(-c3ccc4oc5cccc6ccc3c4c65)c3ccccc23)c1 ; c1ccc2c(c1)ccc1cc(-c3ccc(-c4c5ccccc5c(-c5ccc6oc7cccc8ccc5c6c87)c5ccccc45)cc3)ccc12 ; c1cc(-c2ccc3c(ccc4ccccc43)c2)cc(-c2c3ccccc3c(-c3ccc4oc5cccc6ccc3c4c65)c3ccccc23)c1]

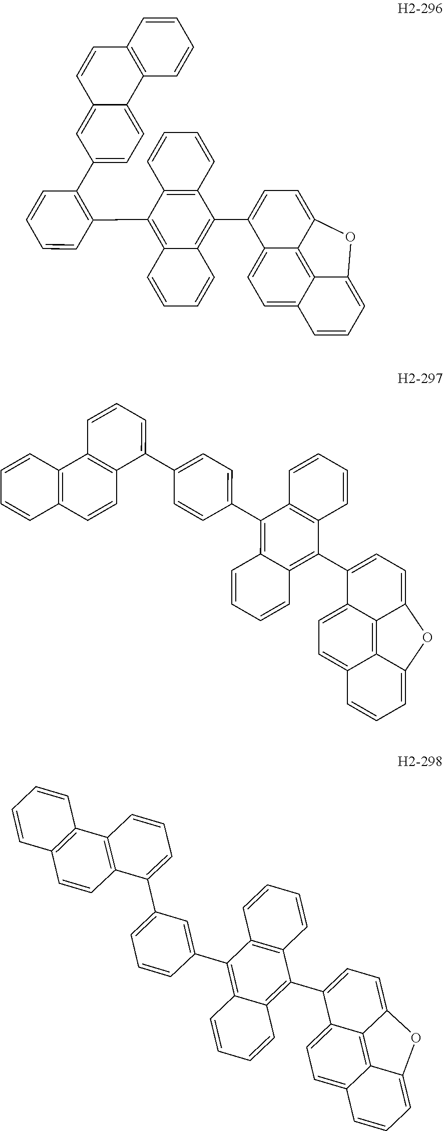 [SMILES: c1ccc(-c2c3ccccc3c(-c3ccc4oc5cccc6ccc3c4c65)c3ccccc23)c(-c2ccc3c(ccc4ccccc43)c2)c1 ; c1ccc2c(c1)ccc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2ccc2ccccc23)c1]

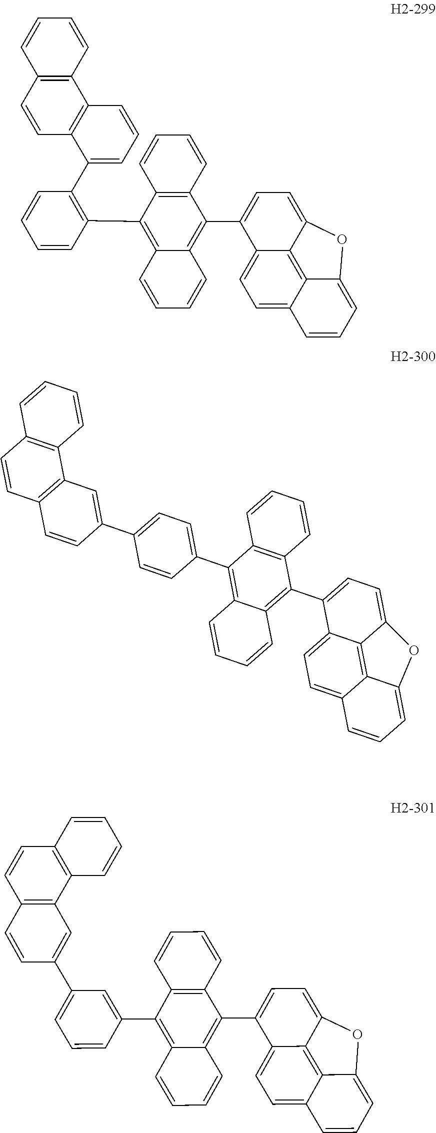 [SMILES: c1ccc(-c2cccc3c2ccc2ccccc23)c(-c2c3ccccc3c(-c3ccc4oc5cccc6ccc3c4c65)c3ccccc23)c1 ; c1ccc2c(c1)ccc1ccc(-c3ccc(-c4c5ccccc5c(-c5ccc6oc7cccc8ccc5c6c87)c5ccccc45)cc3)cc12 ; c1cc(-c2ccc3ccc4ccccc4c3c2)cc(-c2c3ccccc3c(-c3ccc4oc5cccc6ccc3c4c65)c3ccccc23)c1]

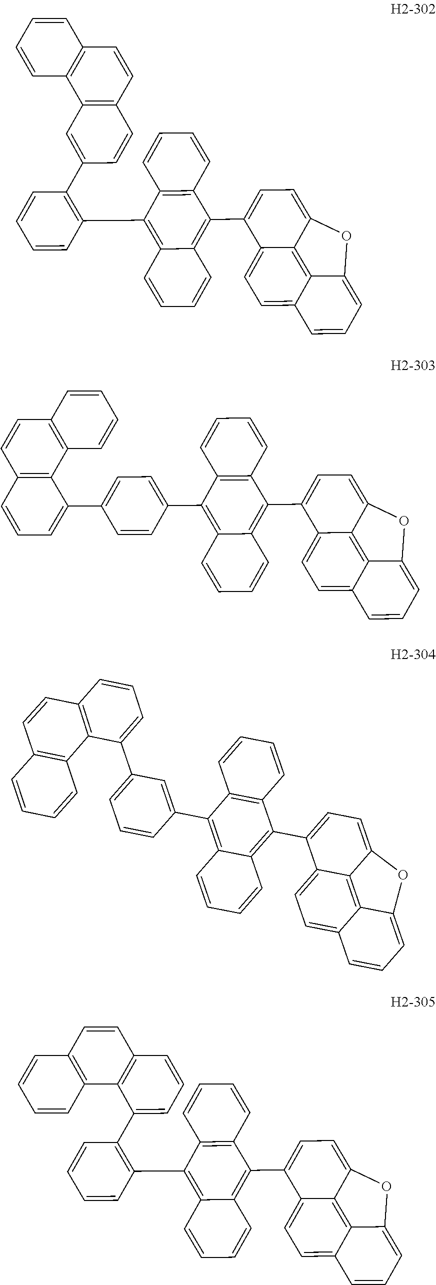 [SMILES: c1ccc(-c2c3ccccc3c(-c3ccc4oc5cccc6ccc3c4c65)c3ccccc23)c(-c2ccc3ccc4ccccc4c3c2)c1 ; c1ccc2c(c1)ccc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3ccc4ccccc4c23)c1 ; c1ccc(-c2cccc3ccc4ccccc4c23)c(-c2c3ccccc3c(-c3ccc4oc5cccc6ccc3c4c65)c3ccccc23)c1]

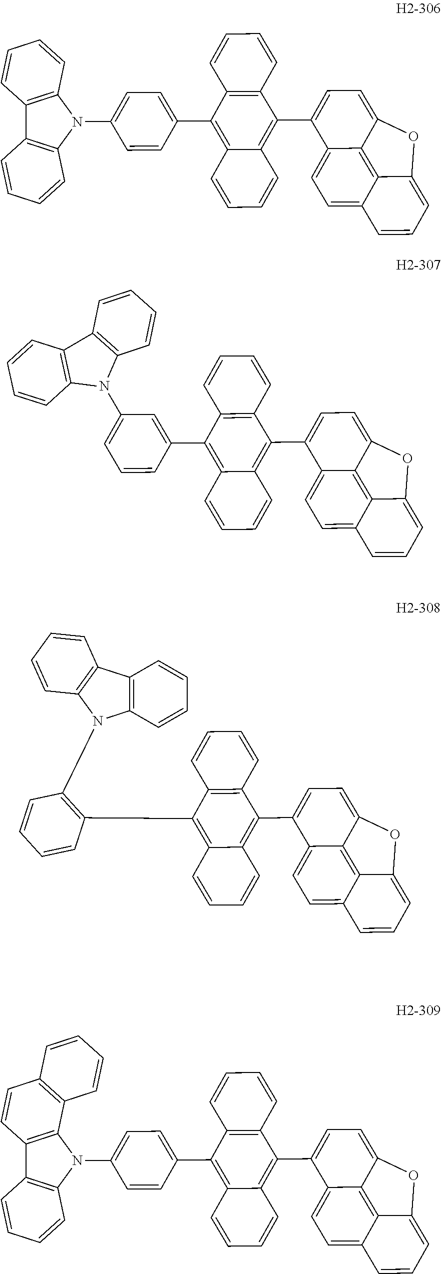 [SMILES: c1cc2ccc3c(-c4c5ccccc5c(-c5ccc(-n6c7ccccc7c7ccccc76)cc5)c5ccccc45)ccc4oc(c1)c2c43 ; c1cc(-c2c3ccccc3c(-c3ccc4oc5cccc6ccc3c4c65)c3ccccc23)cc(-n2c3ccccc3c3ccccc32)c1 ; c1ccc(-n2c3ccccc3c3ccccc32)c(-c2c3ccccc3c(-c3ccc4oc5cccc6ccc3c4c65)c3ccccc23)c1 ; c1ccc2c(c1)ccc1c3ccccc3n(-c3ccc(-c4c5ccccc5c(-c5ccc6oc7cccc8ccc5c6c87)c5ccccc45)cc3)c21]

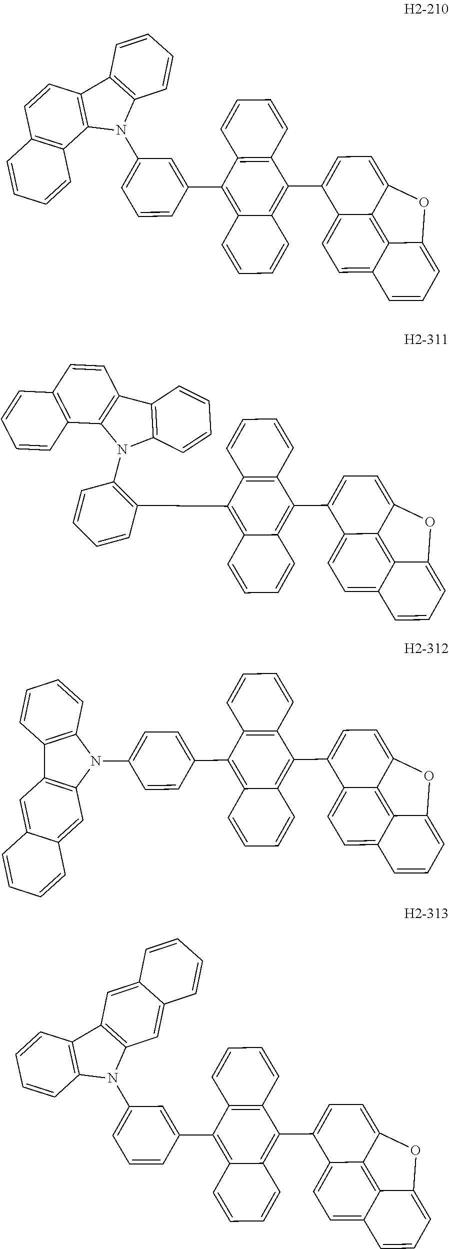 [SMILES: c1cc(-c2c3ccccc3c(-c3ccc4oc5cccc6ccc3c4c65)c3ccccc23)cc(-n2c3ccccc3c3ccc4ccccc4c32)c1 ; c1ccc(-n2c3ccccc3c3ccc4ccccc4c32)c(-c2c3ccccc3c(-c3ccc4oc5cccc6ccc3c4c65)c3ccccc23)c1 ; c1ccc2cc3c(cc2c1)c1ccccc1n3-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3cc4ccccc4cc32)c1]

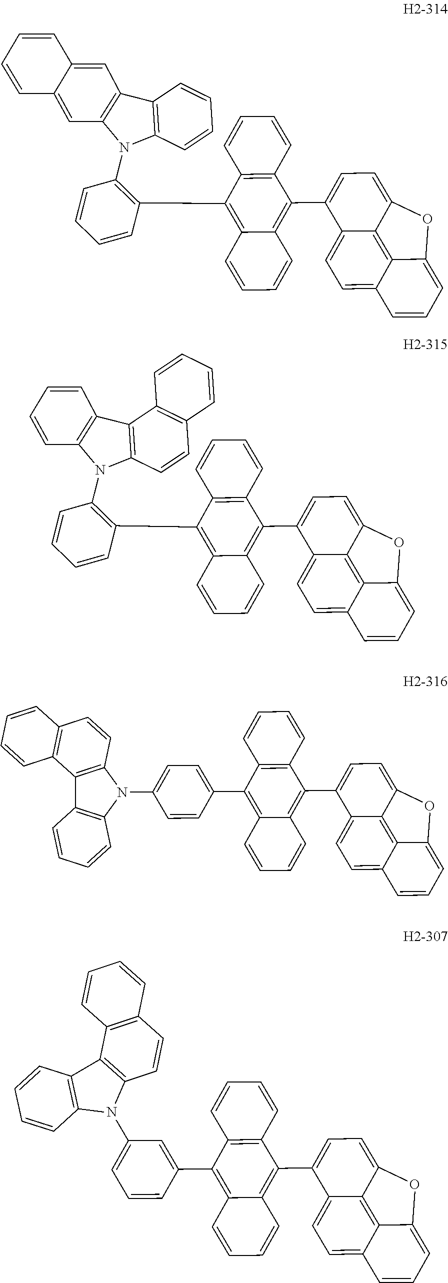 [SMILES: c1ccc(-n2c3ccccc3c3cc4ccccc4cc32)c(-c2c3ccccc3c(-c3ccc4oc5cccc6ccc3c4c65)c3ccccc23)c1 ; c1ccc(-n2c3ccccc3c3c4ccccc4ccc32)c(-c2c3ccccc3c(-c3ccc4oc5cccc6ccc3c4c65)c3ccccc23)c1 ; c1ccc2c(c1)ccc1c2c2ccccc2n1-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3c4ccccc4ccc32)c1]

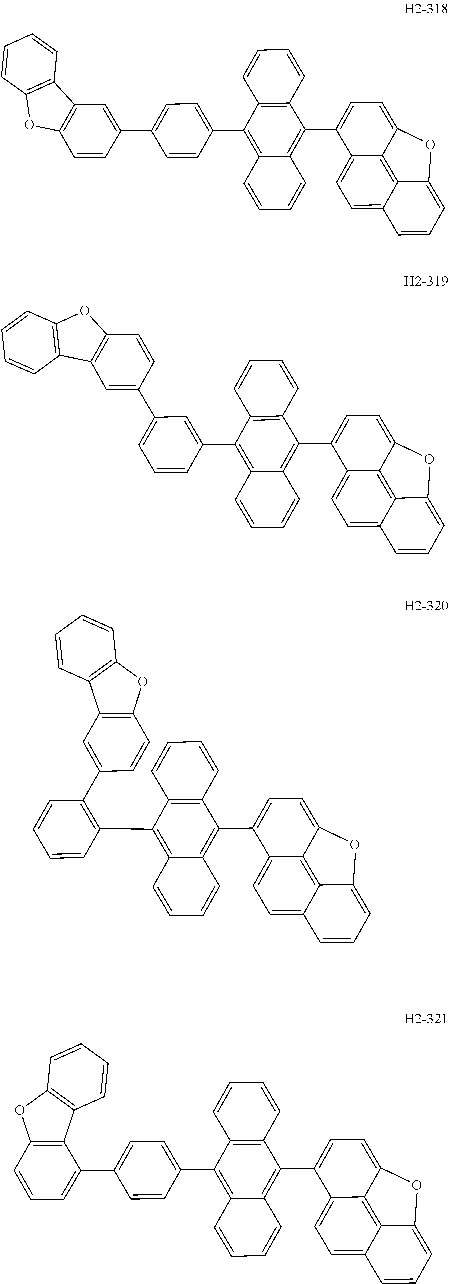 [SMILES: c1ccc2c(c1)oc1ccc(-c3ccc(-c4c5ccccc5c(-c5ccc6oc7cccc8ccc5c6c87)c5ccccc45)cc3)cc12 ; c1cc(-c2ccc3oc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3oc4ccccc4c3c2)c1 ; c1ccc2c(c1)oc1cccc(-c3ccc(-c4c5ccccc5c(-c5ccc6oc7cccc8ccc5c6c87)c5ccccc45)cc3)c12]

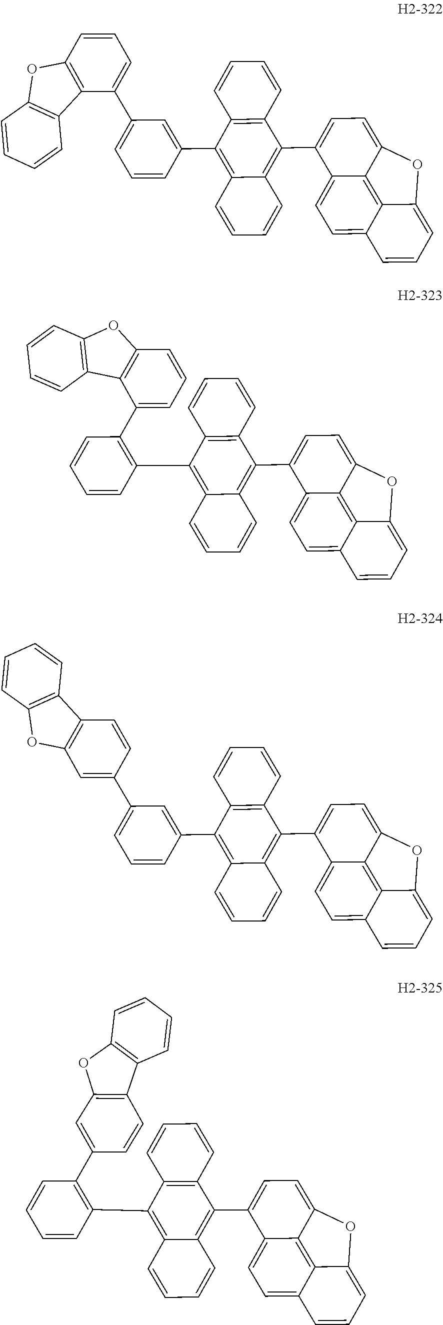 [SMILES: c1cc(-c2c3ccccc3c(-c3ccc4oc5cccc6ccc3c4c65)c3ccccc23)cc(-c2cccc3oc4ccccc4c23)c1 ; c1ccc(-c2cccc3oc4ccccc4c23)c(-c2c3ccccc3c(-c3ccc4oc5cccc6ccc3c4c65)c3ccccc23)c1 ; c1cc(-c2ccc3c(c2)oc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)oc2ccccc23)c1]

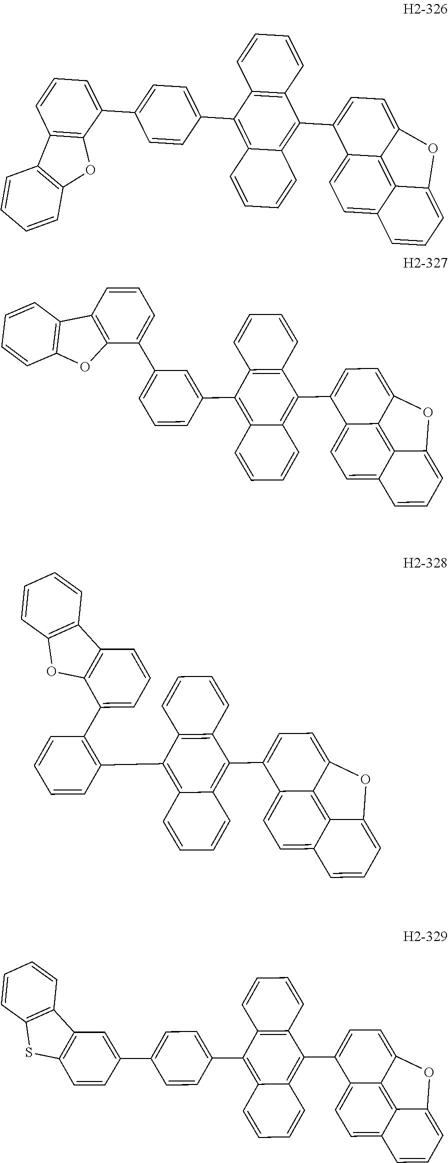 [SMILES: c1ccc2c(c1)oc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2oc2ccccc23)c1 ; c1ccc(-c2cccc3c2oc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6ccc7sc8ccccc8c7c6)cc5)c5ccccc45)ccc4oc(c1)c2c43]

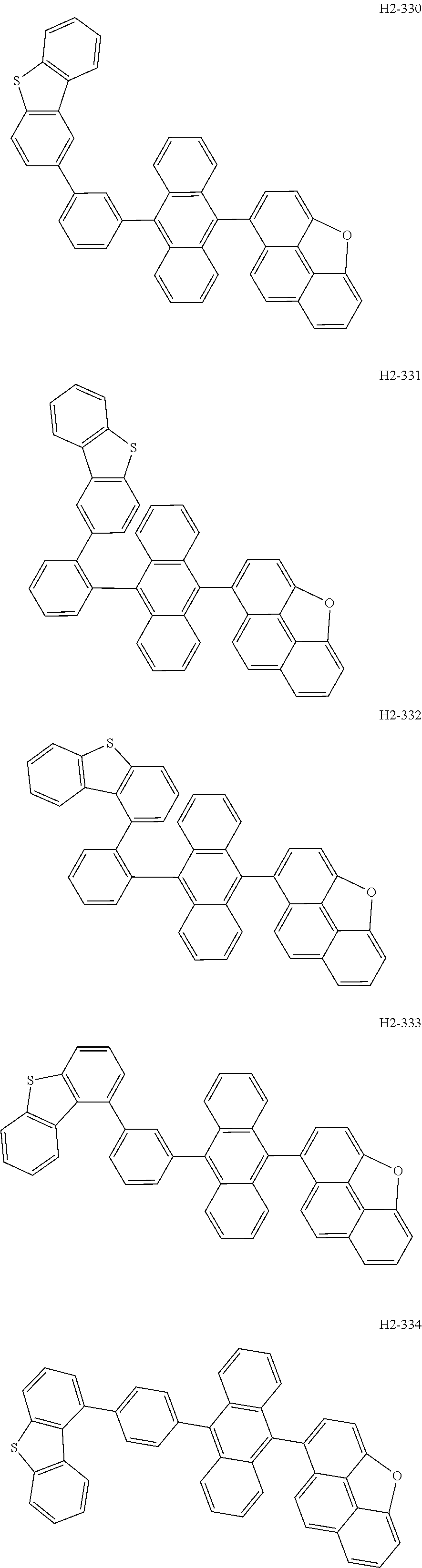 [SMILES: c1cc(-c2ccc3sc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3sc4ccccc4c3c2)c1 ; c1ccc(-c2cccc3sc4ccccc4c23)c(-c2c3ccccc3c(-c3ccc4oc5cccc6ccc3c4c65)c3ccccc23)c1 ; c1cc(-c2c3ccccc3c(-c3ccc4oc5cccc6ccc3c4c65)c3ccccc23)cc(-c2cccc3sc4ccccc4c23)c1 ; c1cc2ccc3c(-c4c5ccccc5c(-c5ccc(-c6cccc7sc8ccccc8c67)cc5)c5ccccc45)ccc4oc(c1)c2c43]

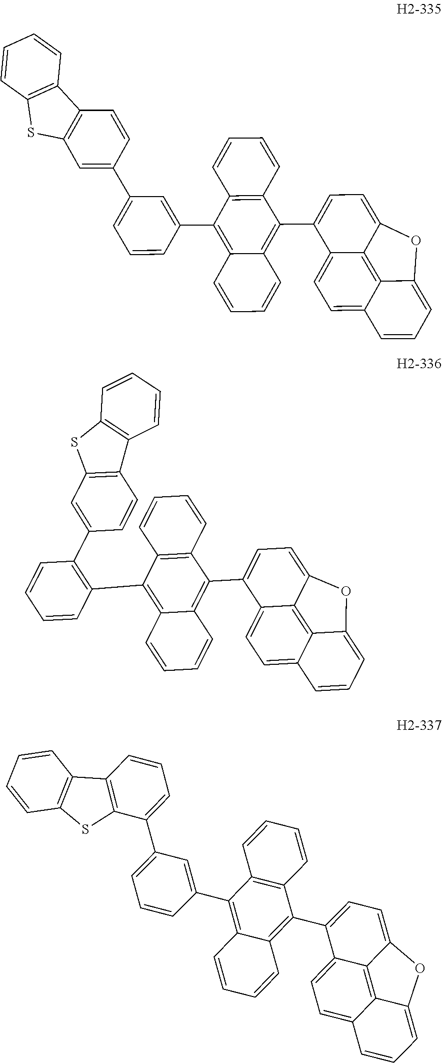 [SMILES: c1cc(-c2ccc3c(c2)sc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)sc2ccccc23)c1 ; c1cc(-c2c3ccccc3c(-c3ccc4oc5cccc6ccc3c4c65)c3ccccc23)cc(-c2cccc3c2sc2ccccc23)c1]

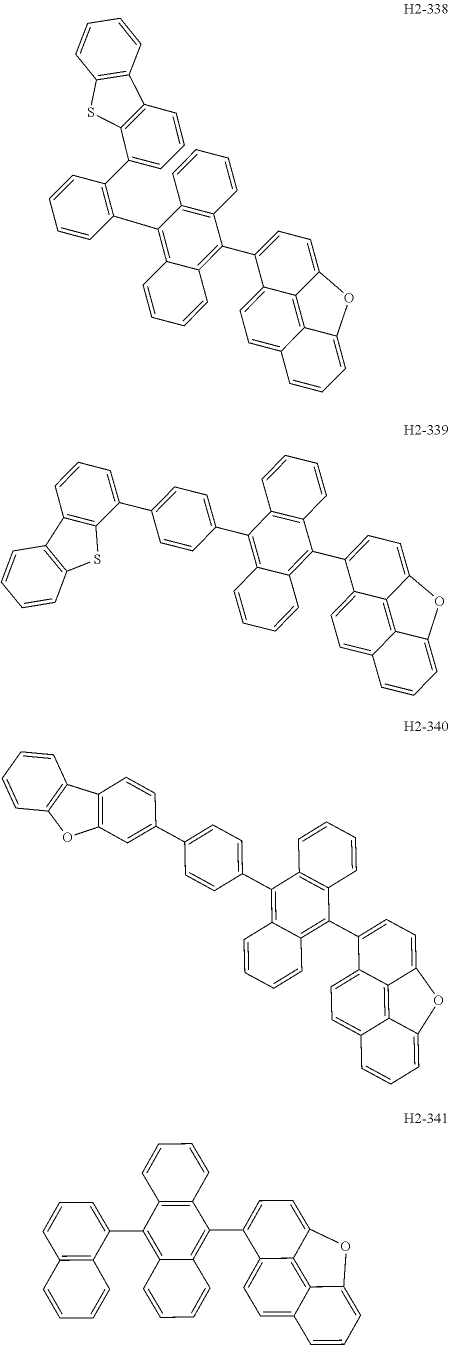 [SMILES: c1ccc(-c2cccc3c2sc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6cccc7c6sc6ccccc67)cc5)c5ccccc45)ccc4oc(c1)c2c43 ; c1ccc2c(c1)oc1cc(-c3ccc(-c4c5ccccc5c(-c5ccc6oc7cccc8ccc5c6c87)c5ccccc45)cc3)ccc12 ; c1ccc2c(-c3c4ccccc4c(-c4ccc5oc6cccc7ccc4c5c76)c4ccccc34)cccc2c1]

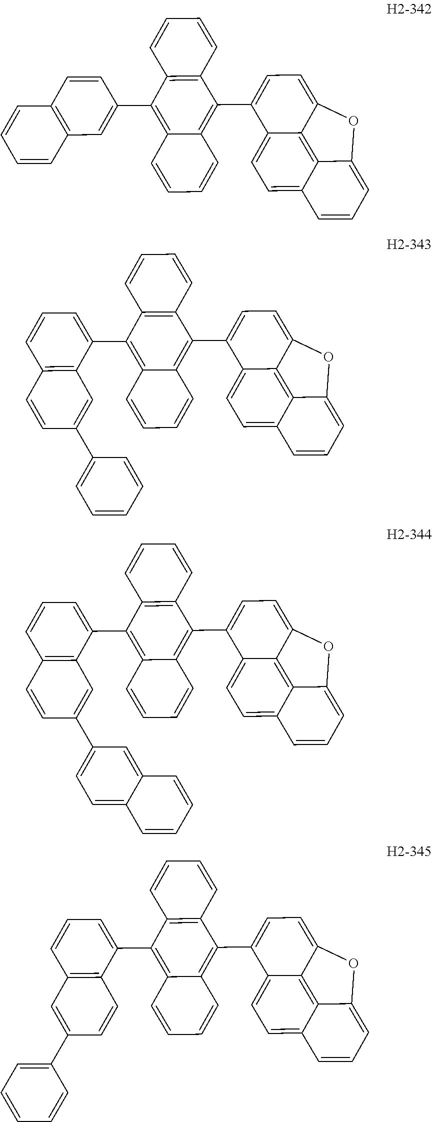

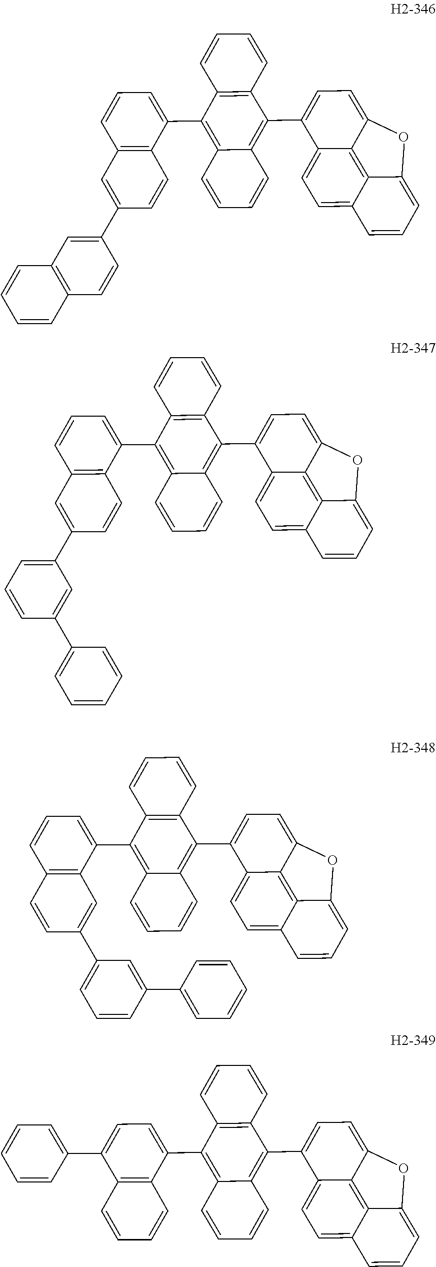 [SMILES: c1ccc2cc(-c3ccc4c(-c5c6ccccc6c(-c6ccc7oc8cccc9ccc6c7c98)c6ccccc56)cccc4c3)ccc2c1 ; c1ccc(-c2cccc(-c3ccc4c(-c5c6ccccc6c(-c6ccc7oc8cccc9ccc6c7c98)c6ccccc56)cccc4c3)c2)cc1 ; c1ccc(-c2cccc(-c3ccc4cccc(-c5c6ccccc6c(-c6ccc7oc8cccc9ccc6c7c98)c6ccccc56)c4c3)c2)cc1 ; c1ccc(-c2ccc(-c3c4ccccc4c(-c4ccc5oc6cccc7ccc4c5c76)c4ccccc34)c3ccccc23)cc1]

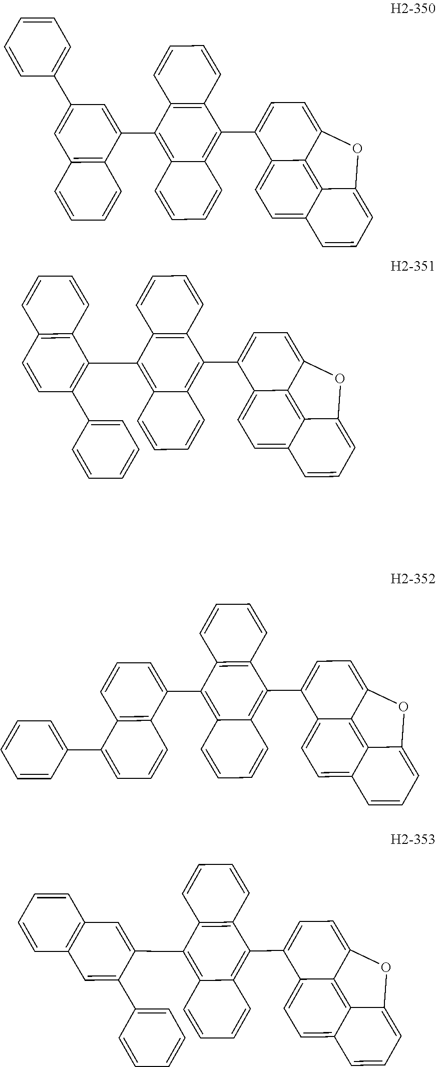 [SMILES: c1ccc(-c2cc(-c3c4ccccc4c(-c4ccc5oc6cccc7ccc4c5c76)c4ccccc34)c3ccccc3c2)cc1 ; c1ccc(-c2ccc3ccccc3c2-c2c3ccccc3c(-c3ccc4oc5cccc6ccc3c4c65)c3ccccc23)cc1 ; c1ccc(-c2cccc3c(-c4c5ccccc5c(-c5ccc6oc7cccc8ccc5c6c87)c5ccccc45)cccc23)cc1 ; c1ccc(-c2cc3ccccc3cc2-c2c3ccccc3c(-c3ccc4oc5cccc6ccc3c4c65)c3ccccc23)cc1]

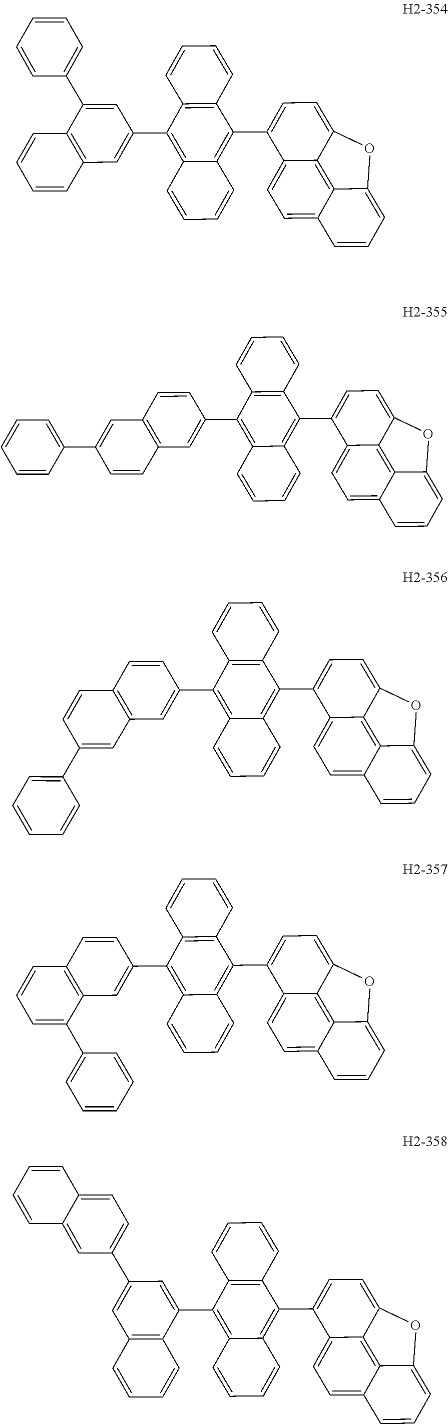 [SMILES: c1ccc(-c2cc(-c3c4ccccc4c(-c4ccc5oc6cccc7ccc4c5c76)c4ccccc34)cc3ccccc23)cc1 ; c1ccc(-c2ccc3cc(-c4c5ccccc5c(-c5ccc6oc7cccc8ccc5c6c87)c5ccccc45)ccc3c2)cc1 ; c1ccc(-c2ccc3ccc(-c4c5ccccc5c(-c5ccc6oc7cccc8ccc5c6c87)c5ccccc45)cc3c2)cc1 ; c1ccc(-c2cccc3ccc(-c4c5ccccc5c(-c5ccc6oc7cccc8ccc5c6c87)c5ccccc45)cc23)cc1 ; c1ccc2cc(-c3cc(-c4c5ccccc5c(-c5ccc6oc7cccc8ccc5c6c87)c5ccccc45)c4ccccc4c3)ccc2c1]

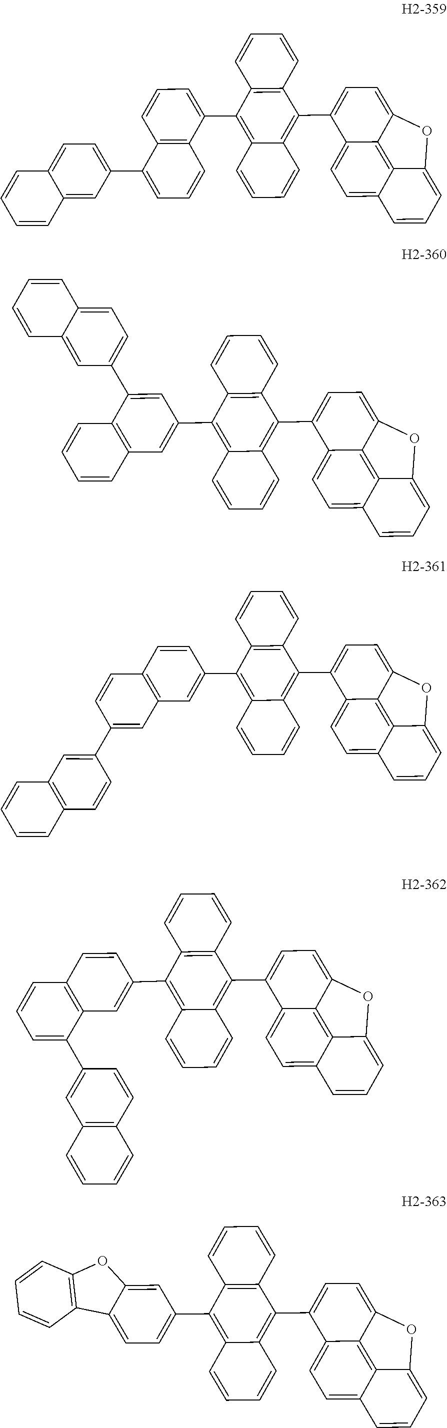 [SMILES: c1ccc2cc(-c3cccc4c(-c5c6ccccc6c(-c6ccc7oc8cccc9ccc6c7c98)c6ccccc56)cccc34)ccc2c1 ; c1ccc2cc(-c3cc(-c4c5ccccc5c(-c5ccc6oc7cccc8ccc5c6c87)c5ccccc45)cc4ccccc34)ccc2c1 ; c1ccc2cc(-c3ccc4ccc(-c5c6ccccc6c(-c6ccc7oc8cccc9ccc6c7c98)c6ccccc56)cc4c3)ccc2c1 ; c1ccc2cc(-c3cccc4ccc(-c5c6ccccc6c(-c6ccc7oc8cccc9ccc6c7c98)c6ccccc56)cc34)ccc2c1 ; c1ccc2c(c1)oc1cc(-c3c4ccccc4c(-c4ccc5oc6cccc7ccc4c5c76)c4ccccc34)ccc12]

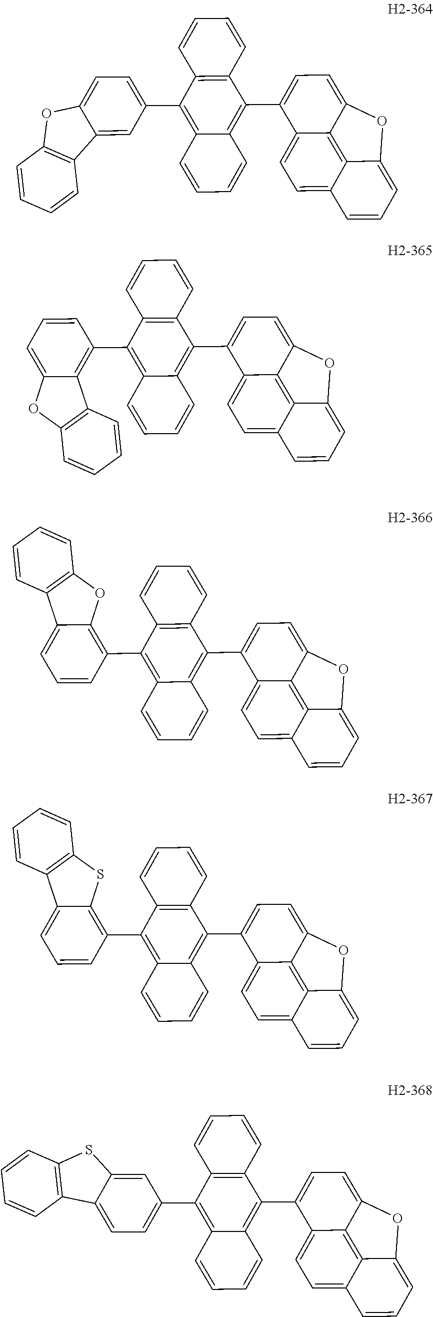 [SMILES: c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12 ; c1ccc2c(c1)oc1c(-c3c4ccccc4c(-c4ccc5oc6cccc7ccc4c5c76)c4ccccc34)cccc12 ; c1cc2ccc3c(-c4c5ccccc5c(-c5cccc6c5sc5ccccc56)c5ccccc45)ccc4oc(c1)c2c43 ; c1cc2ccc3c(-c4c5ccccc5c(-c5ccc6c(c5)sc5ccccc56)c5ccccc45)ccc4oc(c1)c2c43]

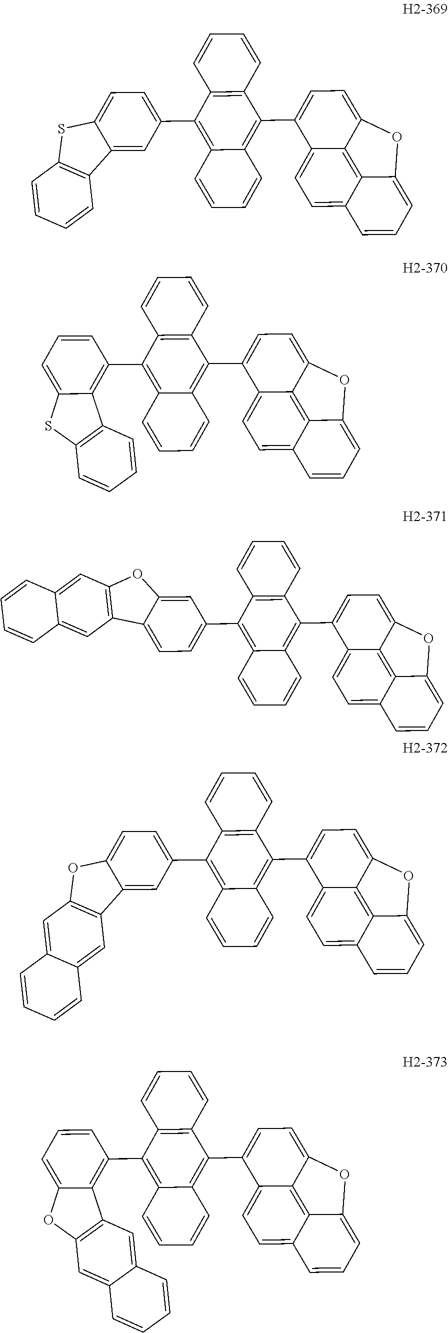 [SMILES: c1cc2ccc3c(-c4c5ccccc5c(-c5ccc6sc7ccccc7c6c5)c5ccccc45)ccc4oc(c1)c2c43 ; c1cc2ccc3c(-c4c5ccccc5c(-c5cccc6sc7ccccc7c56)c5ccccc45)ccc4oc(c1)c2c43 ; c1ccc2cc3c(cc2c1)oc1cc(-c2c4ccccc4c(-c4ccc5oc6cccc7ccc4c5c76)c4ccccc24)ccc13 ; c1ccc2cc3c(cc2c1)oc1ccc(-c2c4ccccc4c(-c4ccc5oc6cccc7ccc4c5c76)c4ccccc24)cc13 ; c1ccc2cc3c(cc2c1)oc1cccc(-c2c4ccccc4c(-c4ccc5oc6cccc7ccc4c5c76)c4ccccc24)c13]

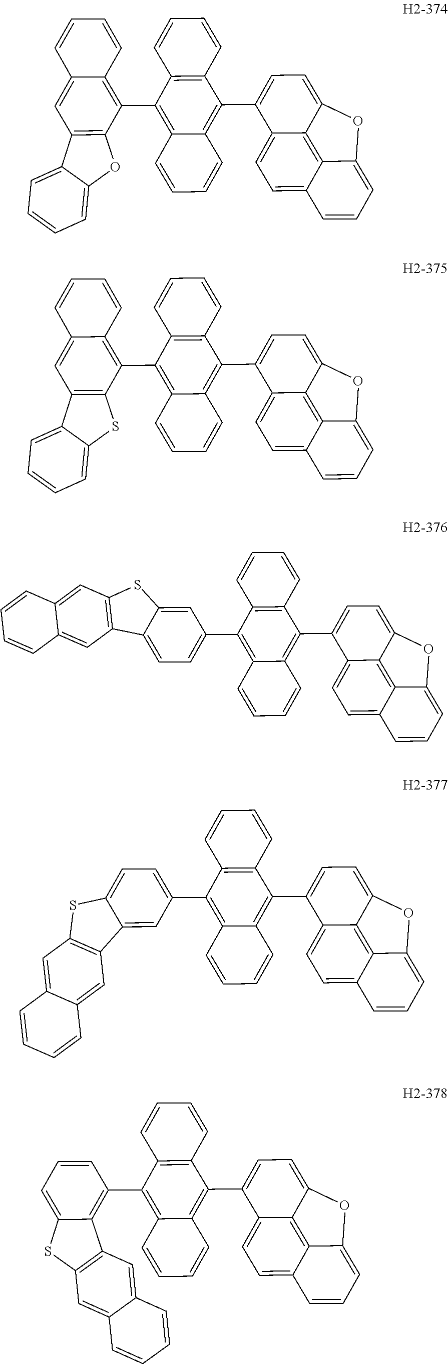 [SMILES: c1ccc2c(-c3c4ccccc4c(-c4ccc5oc6cccc7ccc4c5c76)c4ccccc34)c3oc4ccccc4c3cc2c1 ; c1ccc2c(-c3c4ccccc4c(-c4ccc5oc6cccc7ccc4c5c76)c4ccccc34)c3sc4ccccc4c3cc2c1 ; c1ccc2cc3c(cc2c1)sc1cc(-c2c4ccccc4c(-c4ccc5oc6cccc7ccc4c5c76)c4ccccc24)ccc13 ; c1ccc2cc3c(cc2c1)sc1ccc(-c2c4ccccc4c(-c4ccc5oc6cccc7ccc4c5c76)c4ccccc24)cc13 ; c1ccc2cc3c(cc2c1)sc1cccc(-c2c4ccccc4c(-c4ccc5oc6cccc7ccc4c5c76)c4ccccc24)c13]

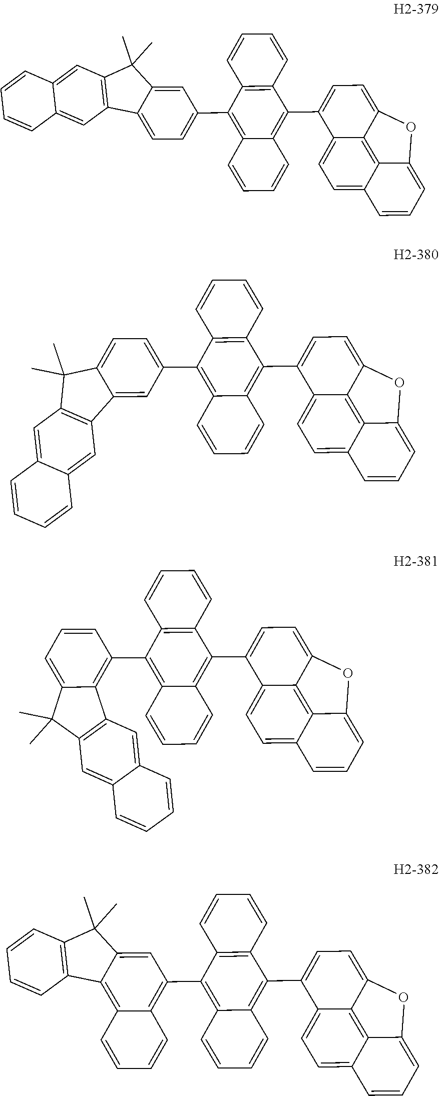 [SMILES: CC1(C)c2cc(-c3c4ccccc4c(-c4ccc5oc6cccc7ccc4c5c76)c4ccccc34)ccc2-c2cc3ccccc3cc21 ; CC1(C)c2ccc(-c3c4ccccc4c(-c4ccc5oc6cccc7ccc4c5c76)c4ccccc34)cc2-c2cc3ccccc3cc21 ; CC1(C)c2cc3ccccc3cc2-c2c(-c3c4ccccc4c(-c4ccc5oc6cccc7ccc4c5c76)c4ccccc34)cccc21 ; CC1(C)c2ccccc2-c2c1cc(-c1c3ccccc3c(-c3ccc4oc5cccc6ccc3c4c65)c3ccccc13)c1ccccc21]

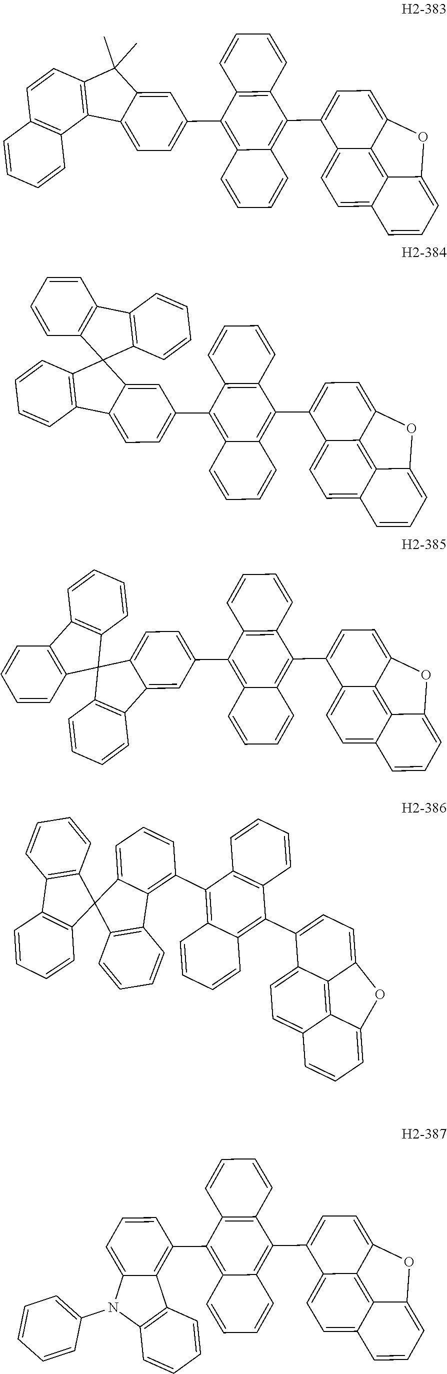 [SMILES: CC1(C)c2cc(-c3c4ccccc4c(-c4ccc5oc6cccc7ccc4c5c76)c4ccccc34)ccc2-c2c1ccc1ccccc21 ; c1ccc2c(c1)-c1ccccc1C21c2ccccc2-c2ccc(-c3c4ccccc4c(-c4ccc5oc6cccc7ccc4c5c76)c4ccccc34)cc21 ; c1ccc2c(c1)-c1ccccc1C21c2ccccc2-c2cc(-c3c4ccccc4c(-c4ccc5oc6cccc7ccc4c5c76)c4ccccc34)ccc21 ; c1ccc2c(c1)-c1ccccc1C21c2ccccc2-c2c(-c3c4ccccc4c(-c4ccc5oc6cccc7ccc4c5c76)c4ccccc34)cccc21 ; c1ccc(-n2c3ccccc3c3c(-c4c5ccccc5c(-c5ccc6oc7cccc8ccc5c6c87)c5ccccc45)cccc32)cc1]

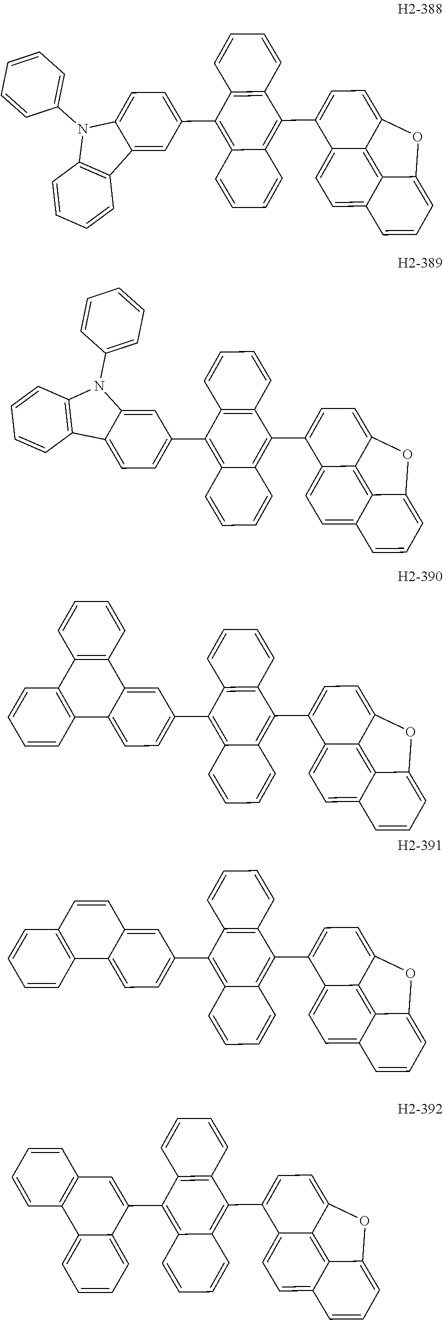 [SMILES: c1ccc(-n2c3ccccc3c3cc(-c4c5ccccc5c(-c5ccc6oc7cccc8ccc5c6c87)c5ccccc45)ccc32)cc1 ; c1ccc(-n2c3ccccc3c3ccc(-c4c5ccccc5c(-c5ccc6oc7cccc8ccc5c6c87)c5ccccc45)cc32)cc1 ; c1cc2ccc3c(-c4c5ccccc5c(-c5ccc6c7ccccc7c7ccccc7c6c5)c5ccccc45)ccc4oc(c1)c2c43 ; c1ccc2c(c1)ccc1cc(-c3c4ccccc4c(-c4ccc5oc6cccc7ccc4c5c76)c4ccccc34)ccc12 ; c1ccc2c(c1)cc(-c1c3ccccc3c(-c3ccc4oc5cccc6ccc3c4c65)c3ccccc13)c1ccccc12]

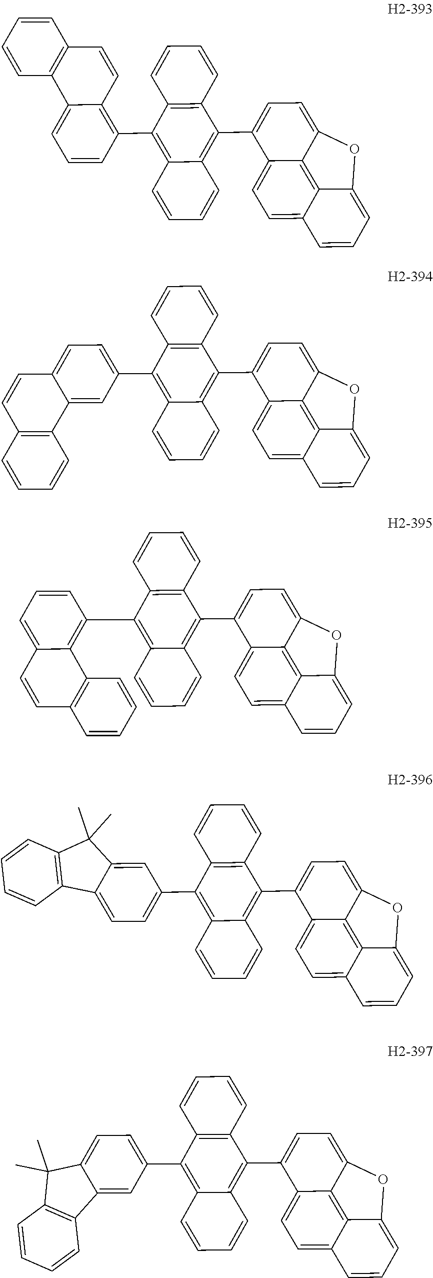 [SMILES: c1ccc2c(c1)ccc1c(-c3c4ccccc4c(-c4ccc5oc6cccc7ccc4c5c76)c4ccccc34)cccc12 ; c1ccc2c(c1)ccc1ccc(-c3c4ccccc4c(-c4ccc5oc6cccc7ccc4c5c76)c4ccccc34)cc12 ; c1ccc2c(c1)ccc1cccc(-c3c4ccccc4c(-c4ccc5oc6cccc7ccc4c5c76)c4ccccc34)c12 ; CC1(C)c2ccccc2-c2ccc(-c3c4ccccc4c(-c4ccc5oc6cccc7ccc4c5c76)c4ccccc34)cc21 ; CC1(C)c2ccccc2-c2cc(-c3c4ccccc4c(-c4ccc5oc6cccc7ccc4c5c76)c4ccccc34)ccc21]

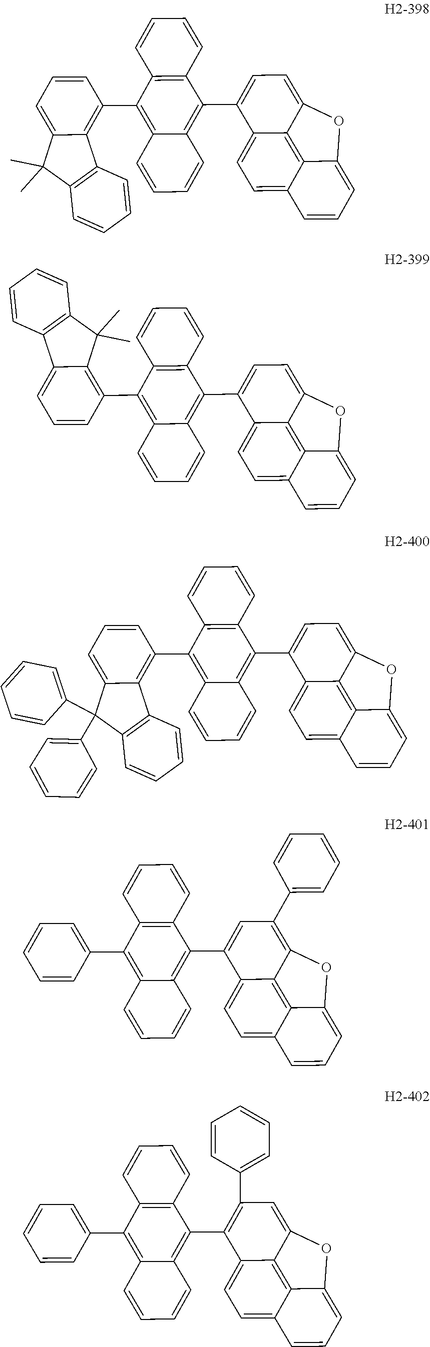 [SMILES: CC1(C)c2ccccc2-c2c(-c3c4ccccc4c(-c4ccc5oc6cccc7ccc4c5c76)c4ccccc34)cccc21 ; CC1(C)c2ccccc2-c2cccc(-c3c4ccccc4c(-c4ccc5oc6cccc7ccc4c5c76)c4ccccc34)c21 ; c1ccc(C2(c3ccccc3)c3ccccc3-c3c(-c4c5ccccc5c(-c5ccc6oc7cccc8ccc5c6c87)c5ccccc45)cccc32)cc1 ; c1ccc(-c2c3ccccc3c(-c3cc(-c4ccccc4)c4oc5cccc6ccc3c4c65)c3ccccc23)cc1 ; c1ccc(-c2cc3oc4cccc5ccc(c2-c2c6ccccc6c(-c6ccccc6)c6ccccc26)c3c54)cc1]

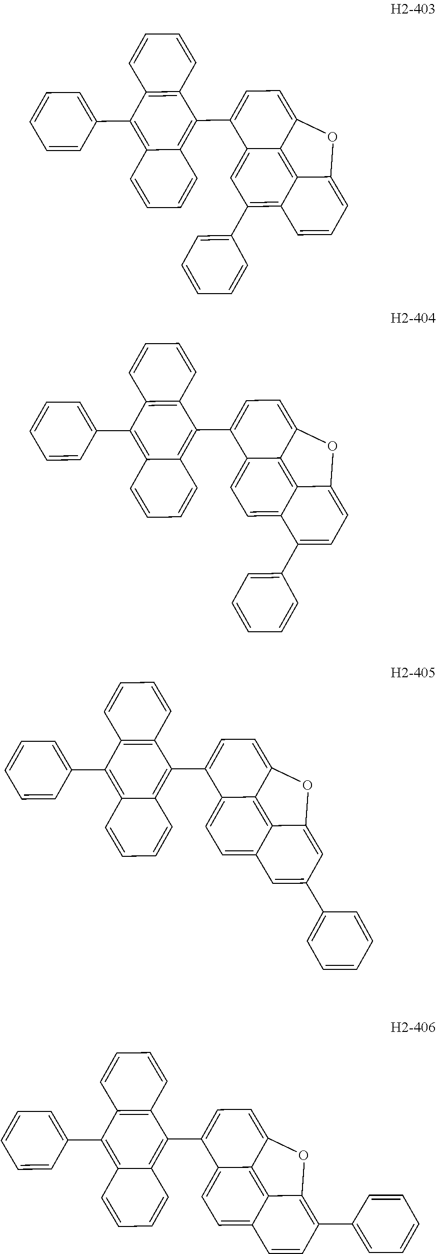 [SMILES: c1ccc(-c2c3ccccc3c(-c3ccc4oc5cccc6c(-c7ccccc7)cc3c4c56)c3ccccc23)cc1 ; c1ccc(-c2c3ccccc3c(-c3ccc4oc5ccc(-c6ccccc6)c6ccc3c4c56)c3ccccc23)cc1 ; c1ccc(-c2cc3ccc4c(-c5c6ccccc6c(-c6ccccc6)c6ccccc56)ccc5oc(c2)c3c54)cc1 ; c1ccc(-c2c3ccccc3c(-c3ccc4oc5c(-c6ccccc6)ccc6ccc3c4c65)c3ccccc23)cc1]

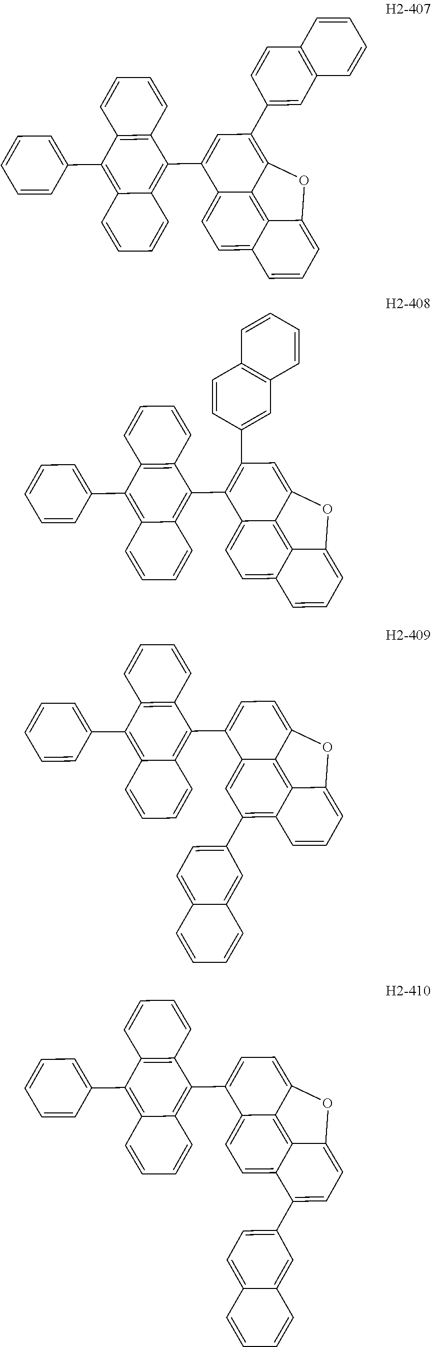

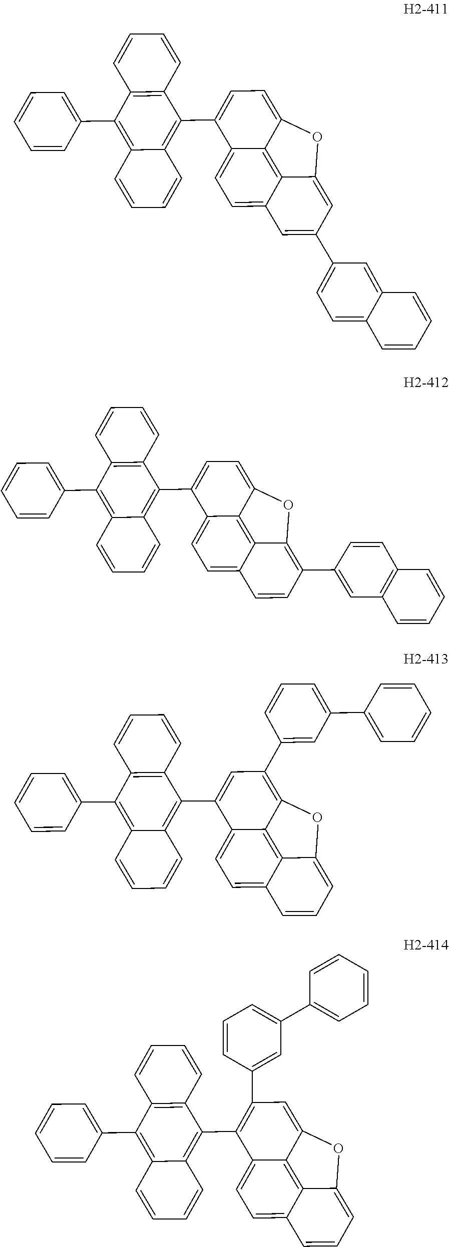

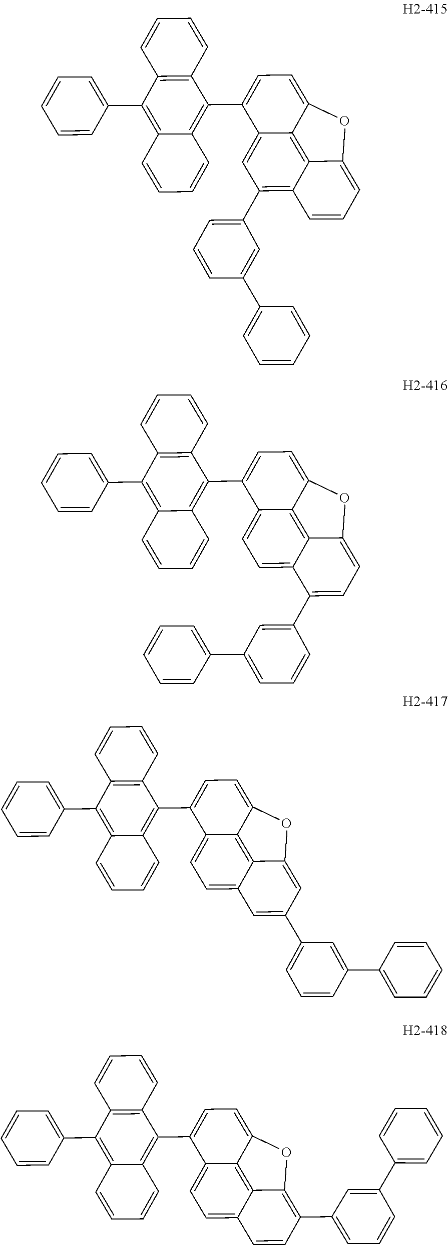 [SMILES: c1ccc(-c2cccc(-c3cc4c(-c5c6ccccc6c(-c6ccccc6)c6ccccc56)ccc5oc6cccc3c6c54)c2)cc1 ; c1ccc(-c2cccc(-c3ccc4oc5ccc(-c6c7ccccc7c(-c7ccccc7)c7ccccc67)c6ccc3c4c56)c2)cc1 ; c1ccc(-c2cccc(-c3cc4ccc5c(-c6c7ccccc7c(-c7ccccc7)c7ccccc67)ccc6oc(c3)c4c65)c2)cc1 ; c1ccc(-c2cccc(-c3ccc4ccc5c(-c6c7ccccc7c(-c7ccccc7)c7ccccc67)ccc6oc3c4c65)c2)cc1]

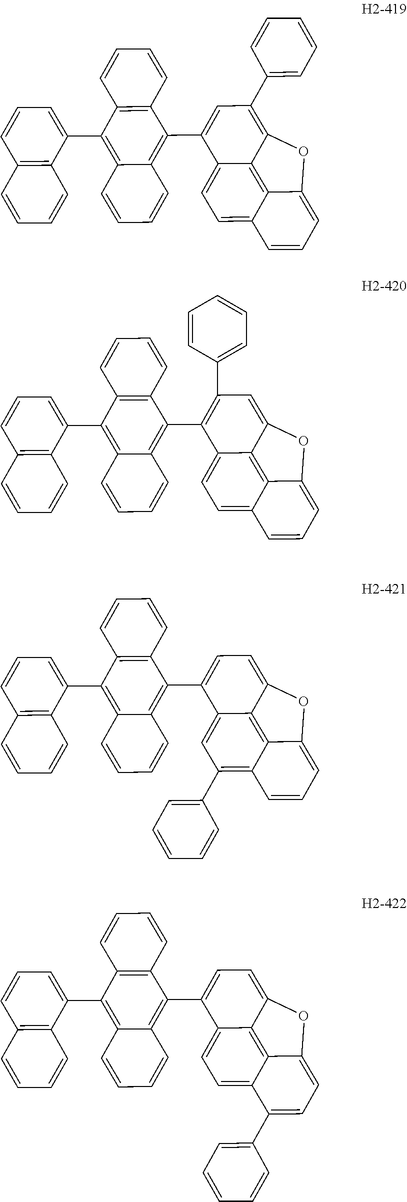 [SMILES: c1ccc(-c2cc(-c3c4ccccc4c(-c4cccc5ccccc45)c4ccccc34)c3ccc4cccc5oc2c3c45)cc1 ; c1ccc(-c2cc3oc4cccc5ccc(c2-c2c6ccccc6c(-c6cccc7ccccc67)c6ccccc26)c3c54)cc1 ; c1ccc(-c2cc3c(-c4c5ccccc5c(-c5cccc6ccccc56)c5ccccc45)ccc4oc5cccc2c5c43)cc1 ; c1ccc(-c2ccc3oc4ccc(-c5c6ccccc6c(-c6cccc7ccccc67)c6ccccc56)c5ccc2c3c45)cc1]

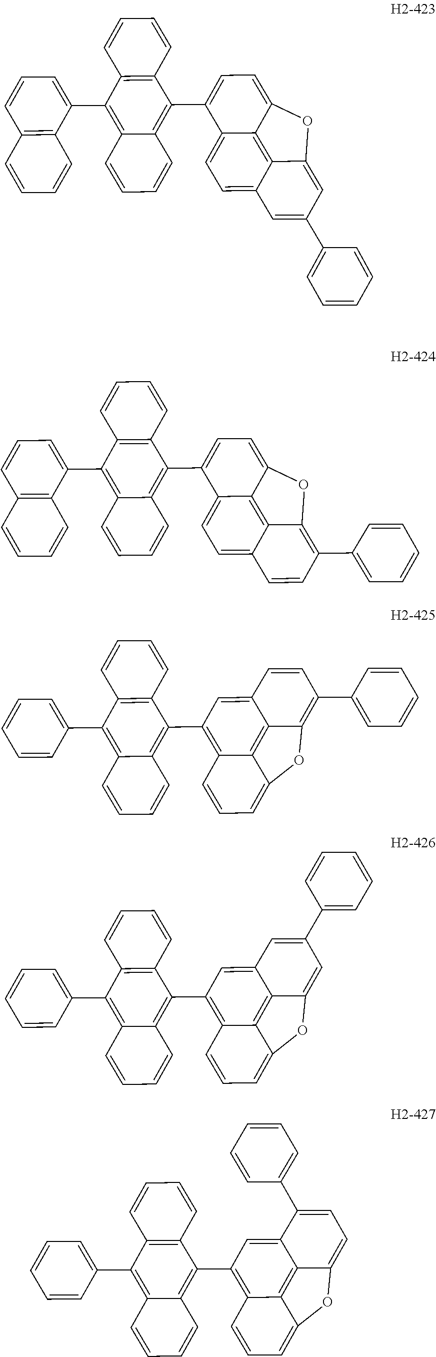

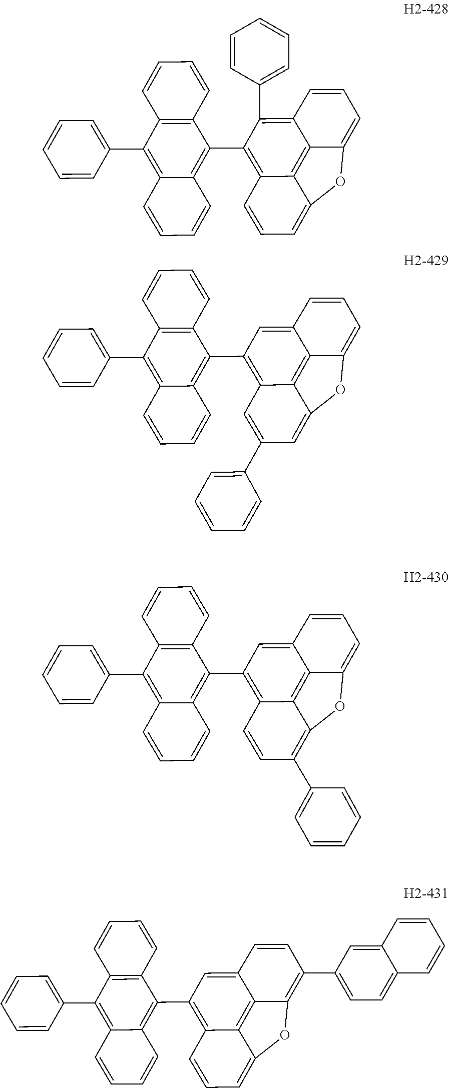

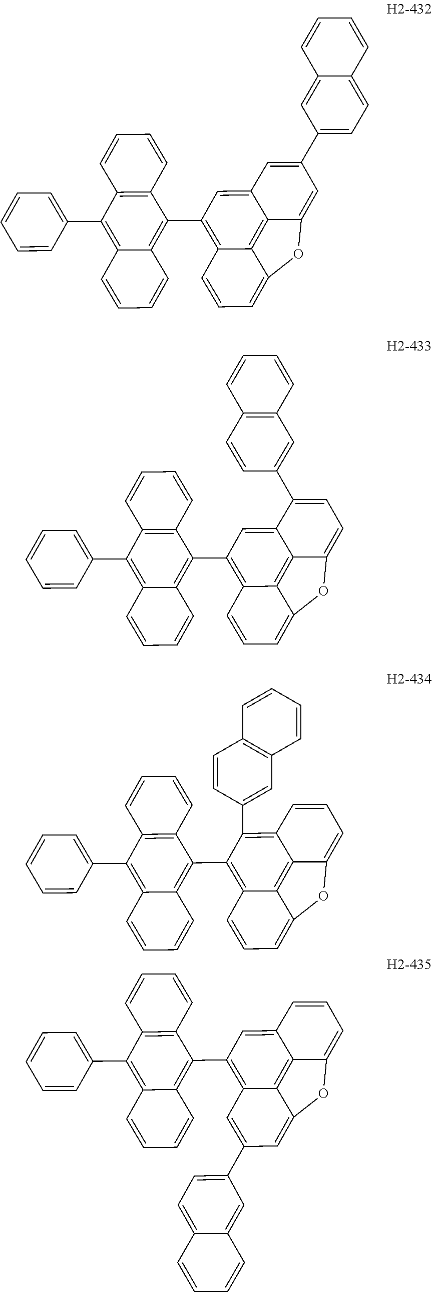

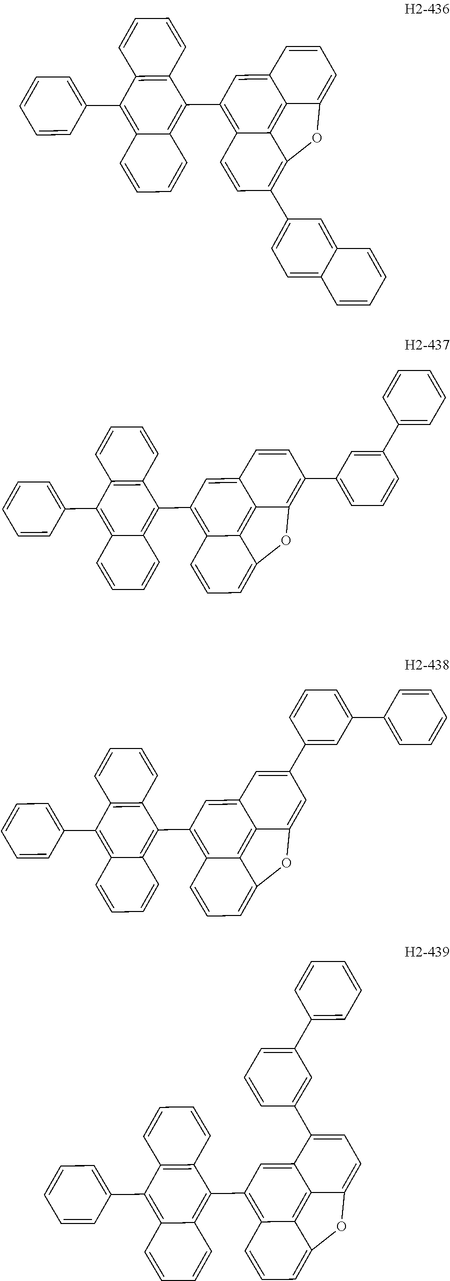

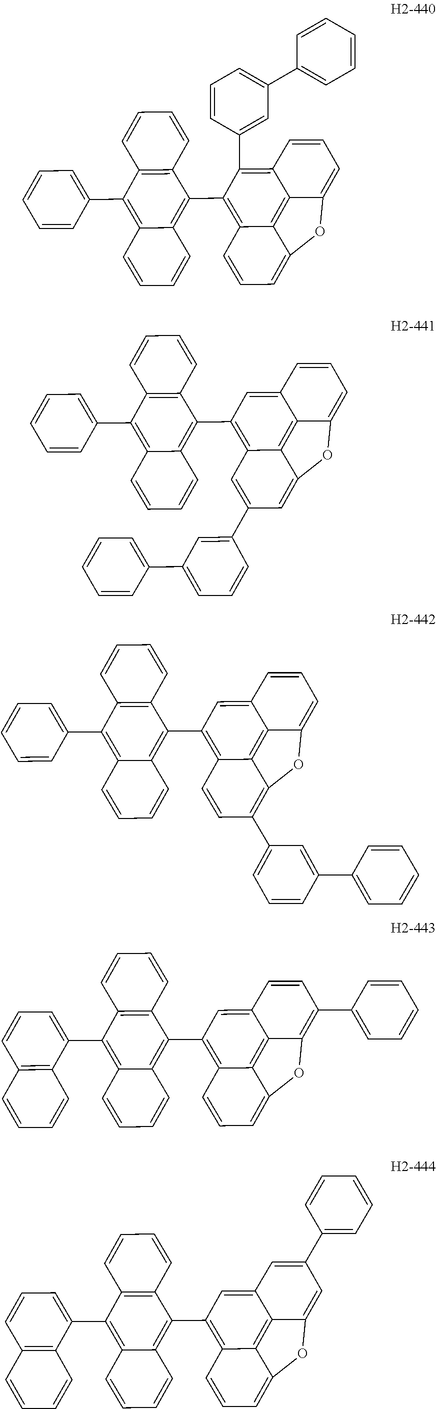

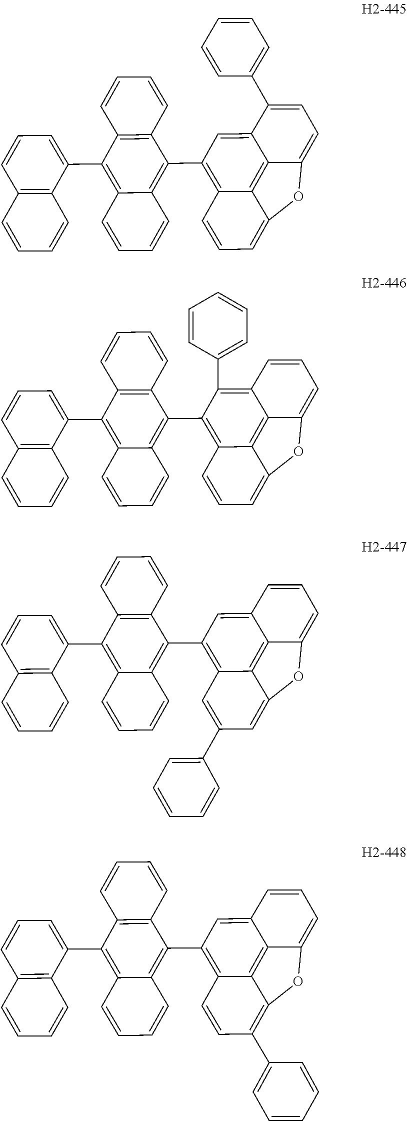

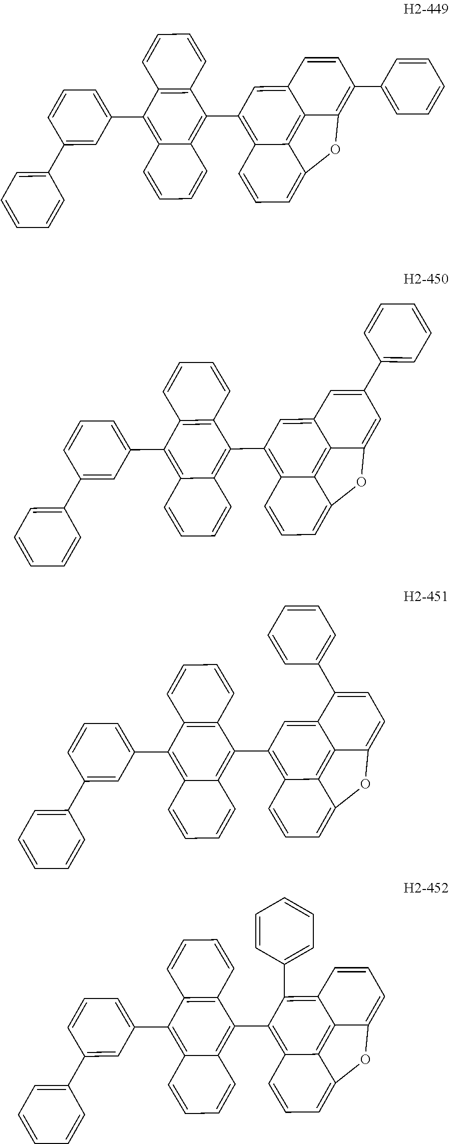

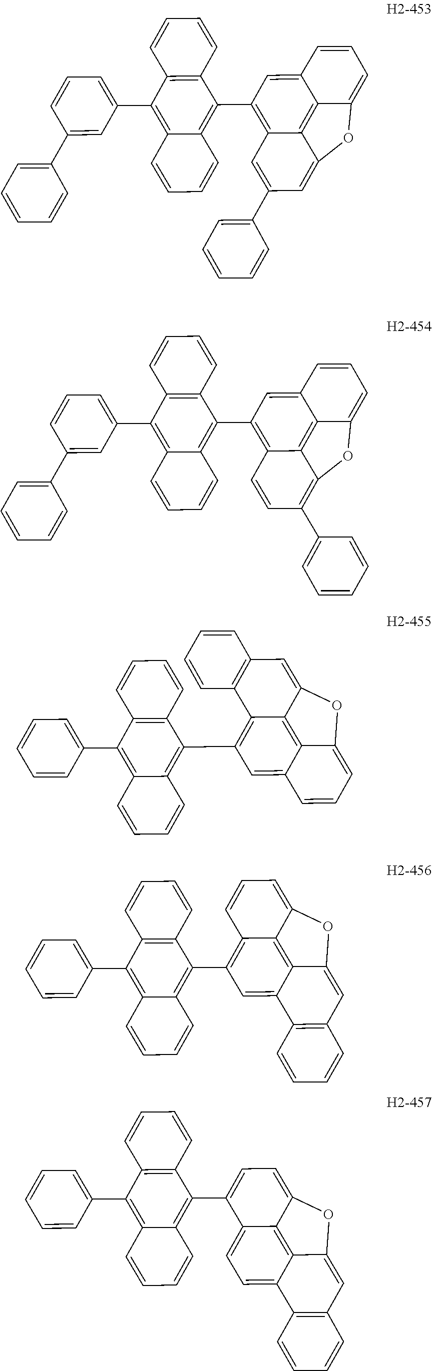

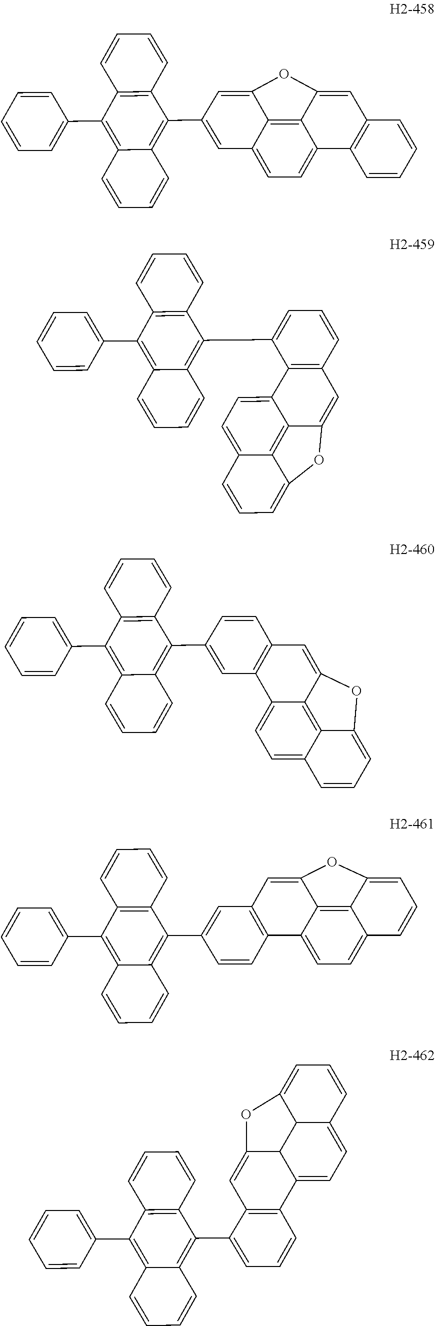 [SMILES: c1ccc(-c2c3ccccc3c(-c3cc4ccc5c6ccccc6cc6oc(c3)c4c65)c3ccccc23)cc1 ; c1ccc(-c2c3ccccc3c(-c3cccc4cc5oc6cccc7ccc(c34)c5c76)c3ccccc23)cc1 ; c1ccc(-c2c3ccccc3c(-c3ccc4cc5oc6cccc7ccc(c4c3)c5c76)c3ccccc23)cc1 ; c1ccc(-c2c3ccccc3c(-c3ccc4c(c3)cc3oc5cccc6ccc4c3c65)c3ccccc23)cc1 ; C1=CC2=CC=C3c4cccc(-c5c6ccccc6c(-c6ccccc6)c6ccccc56)c4C=C4OC(=C1)C2C43]

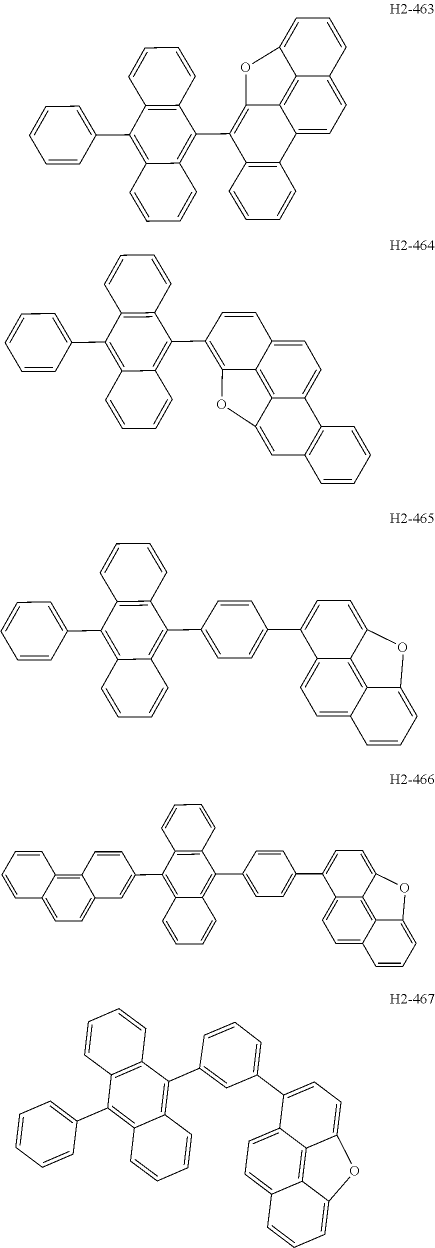

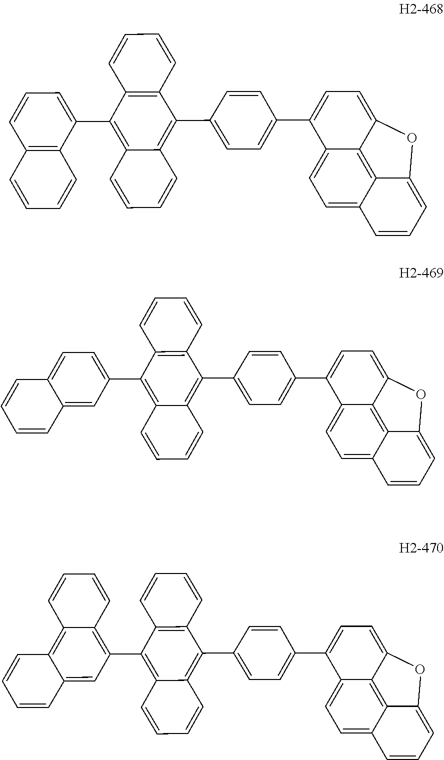 [SMILES: c1ccc2c(-c3c4ccccc4c(-c4ccc(-c5ccc6oc7cccc8ccc5c6c87)cc4)c4ccccc34)cccc2c1 ; c1ccc2cc(-c3c4ccccc4c(-c4ccc(-c5ccc6oc7cccc8ccc5c6c87)cc4)c4ccccc34)ccc2c1 ; c1ccc2c(c1)cc(-c1c3ccccc3c(-c3ccc(-c4ccc5oc6cccc7ccc4c5c76)cc3)c3ccccc13)c1ccccc12]

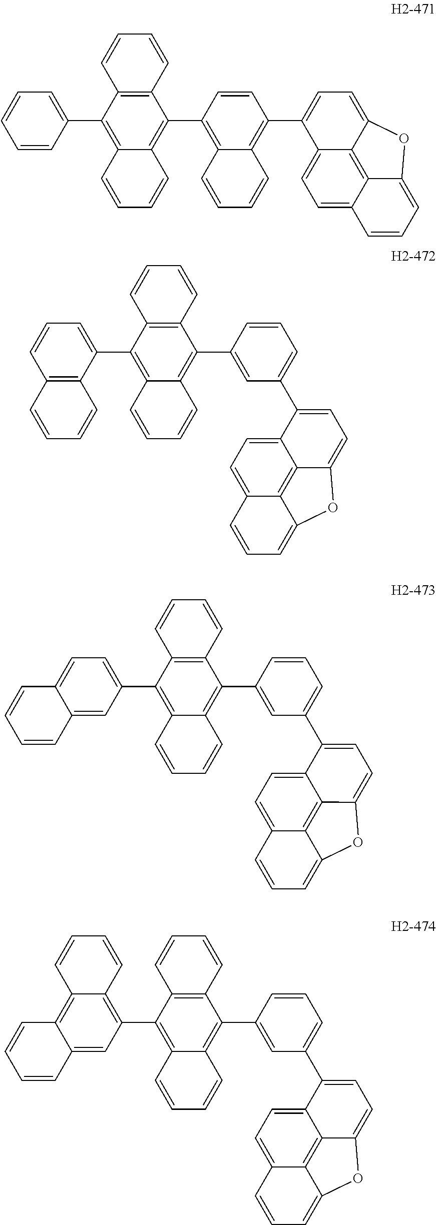 [SMILES: c1ccc(-c2c3ccccc3c(-c3ccc(-c4ccc5oc6cccc7ccc4c5c76)c4ccccc34)c3ccccc23)cc1 ; c1cc(-c2c3ccccc3c(-c3cccc4ccccc34)c3ccccc23)cc(-c2ccc3oc4cccc5ccc2c3c54)c1 ; c1cc(-c2c3ccccc3c(-c3ccc4ccccc4c3)c3ccccc23)cc(-c2ccc3oc4cccc5ccc2c3c54)c1 ; c1cc(-c2c3ccccc3c(-c3cc4ccccc4c4ccccc34)c3ccccc23)cc(-c2ccc3oc4cccc5ccc2c3c54)c1]

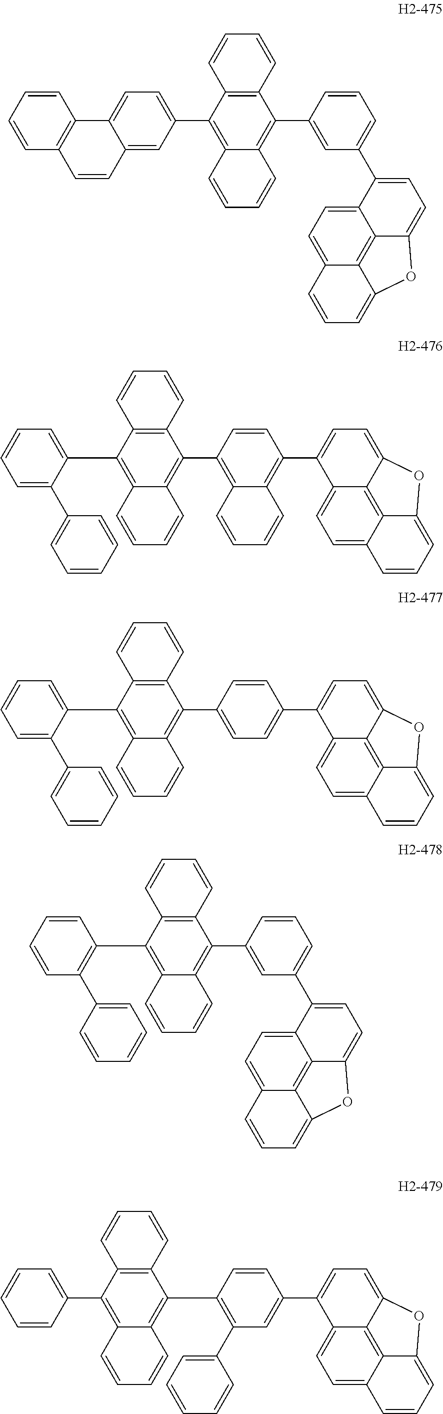 [SMILES: c1cc(-c2c3ccccc3c(-c3ccc4c(ccc5ccccc54)c3)c3ccccc23)cc(-c2ccc3oc4cccc5ccc2c3c54)c1 ; c1ccc(-c2ccccc2-c2c3ccccc3c(-c3ccc(-c4ccc5oc6cccc7ccc4c5c76)c4ccccc34)c3ccccc23)cc1 ; c1ccc(-c2ccccc2-c2c3ccccc3c(-c3ccc(-c4ccc5oc6cccc7ccc4c5c76)cc3)c3ccccc23)cc1 ; c1ccc(-c2ccccc2-c2c3ccccc3c(-c3cccc(-c4ccc5oc6cccc7ccc4c5c76)c3)c3ccccc23)cc1 ; c1ccc(-c2cc(-c3ccc4oc5cccc6ccc3c4c65)ccc2-c2c3ccccc3c(-c3ccccc3)c3ccccc23)cc1]

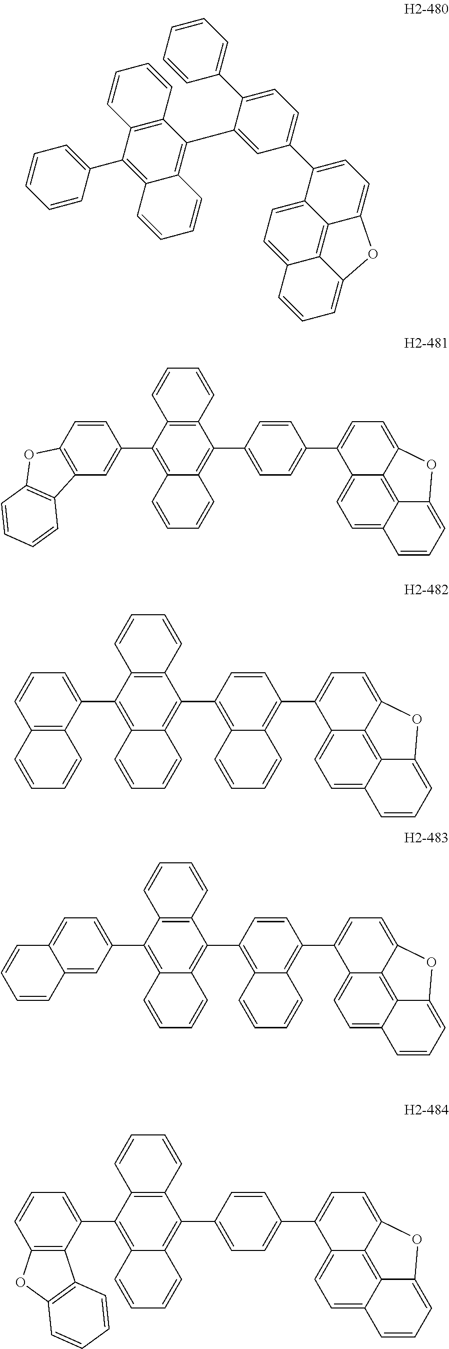 [SMILES: c1ccc(-c2ccc(-c3ccc4oc5cccc6ccc3c4c65)cc2-c2c3ccccc3c(-c3ccccc3)c3ccccc23)cc1 ; c1ccc2c(c1)oc1ccc(-c3c4ccccc4c(-c4ccc(-c5ccc6oc7cccc8ccc5c6c87)cc4)c4ccccc34)cc12 ; c1ccc2c(-c3c4ccccc4c(-c4ccc(-c5ccc6oc7cccc8ccc5c6c87)c5ccccc45)c4ccccc34)cccc2c1 ; c1ccc2cc(-c3c4ccccc4c(-c4ccc(-c5ccc6oc7cccc8ccc5c6c87)c5ccccc45)c4ccccc34)ccc2c1 ; c1ccc2c(c1)oc1cccc(-c3c4ccccc4c(-c4ccc(-c5ccc6oc7cccc8ccc5c6c87)cc4)c4ccccc34)c12]

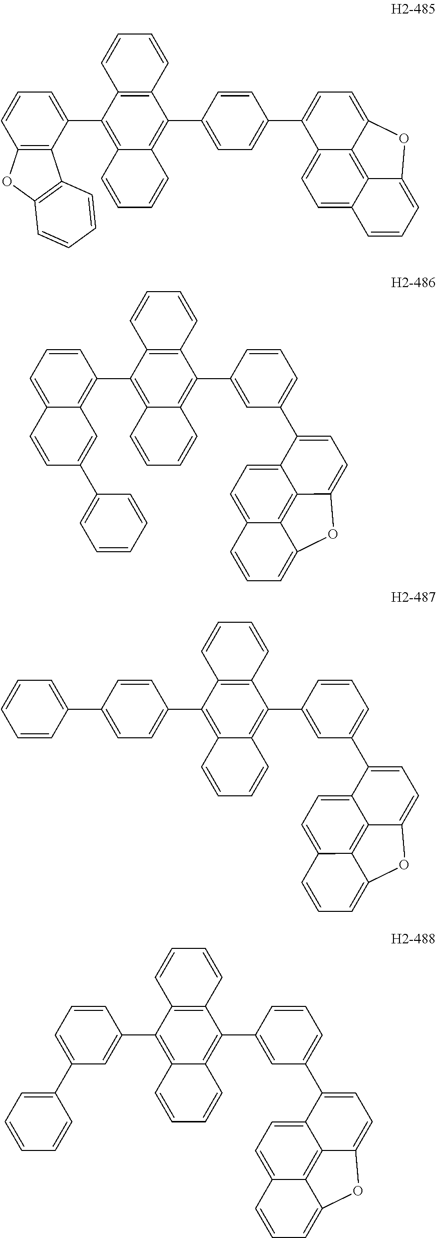

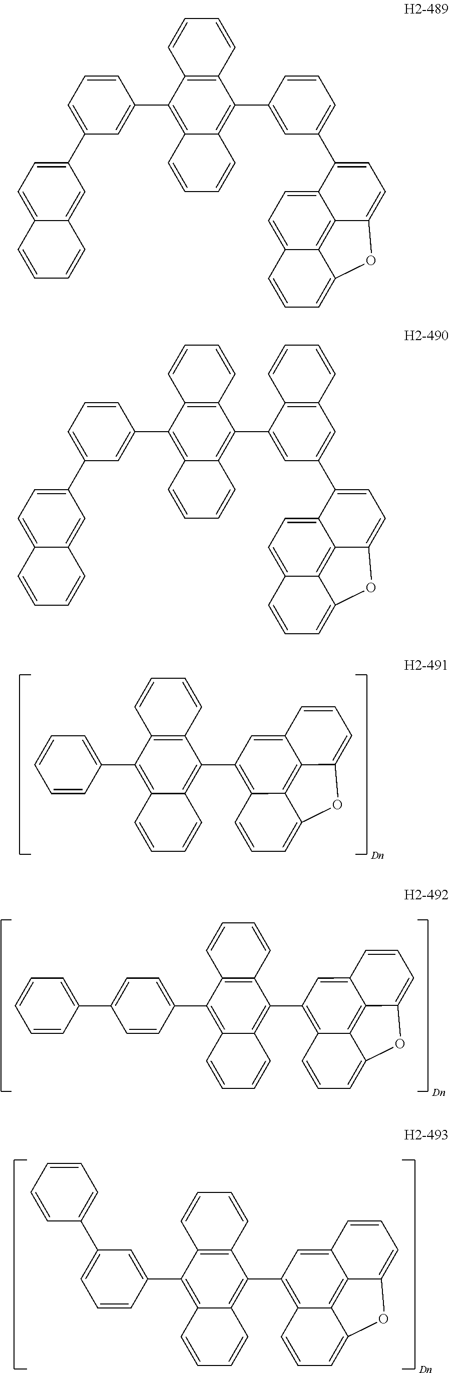

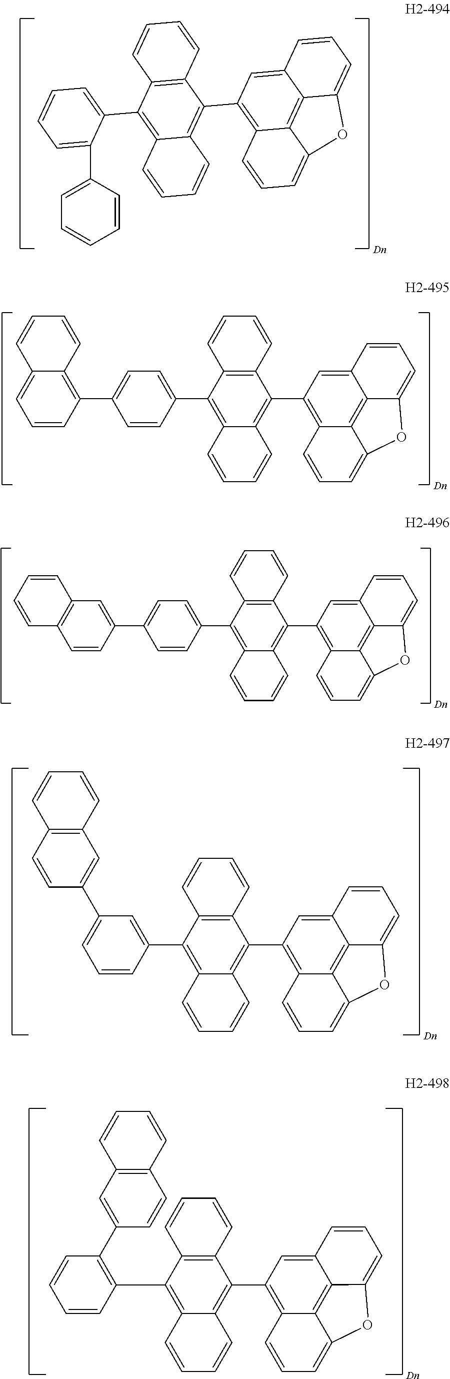

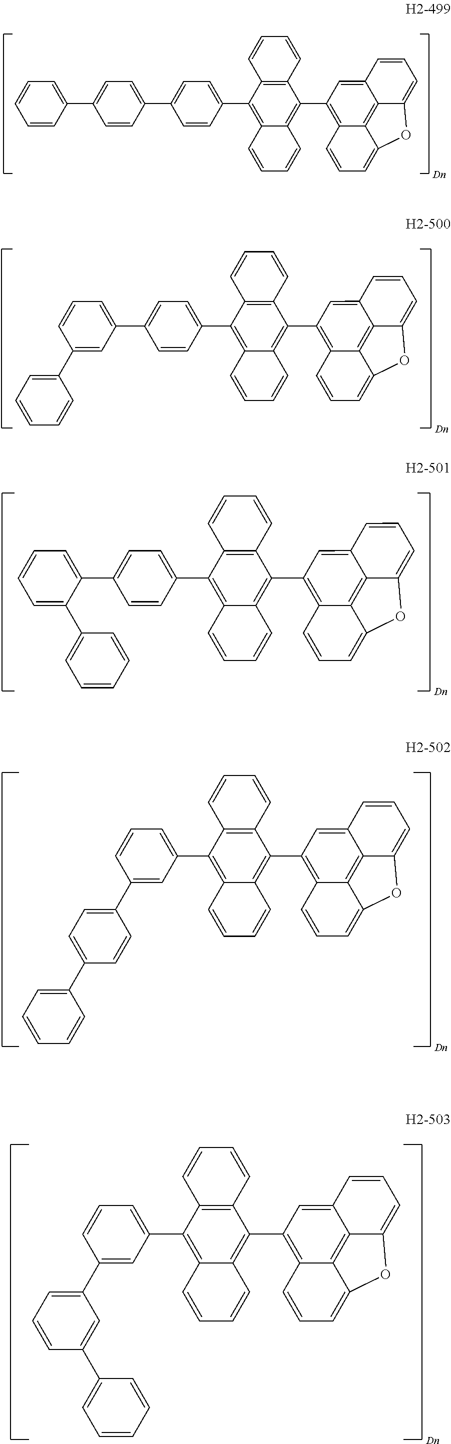

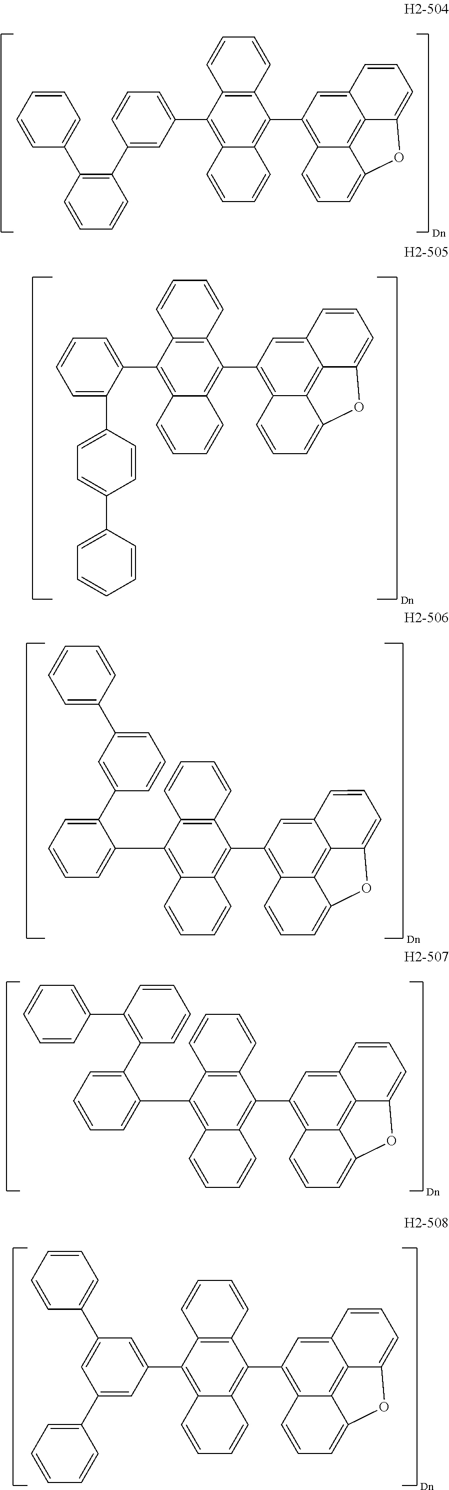

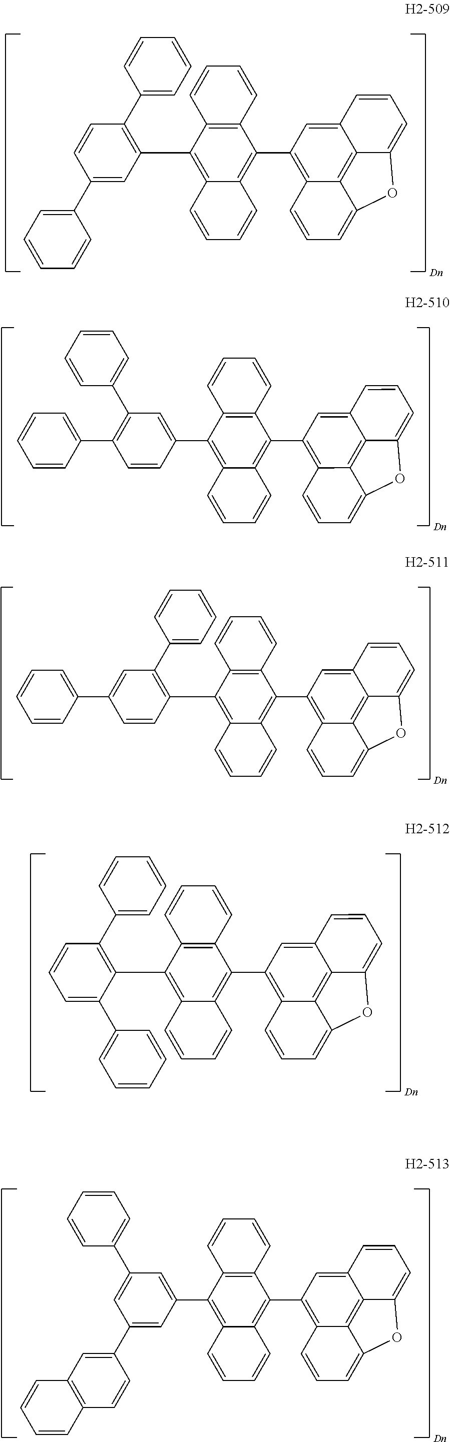

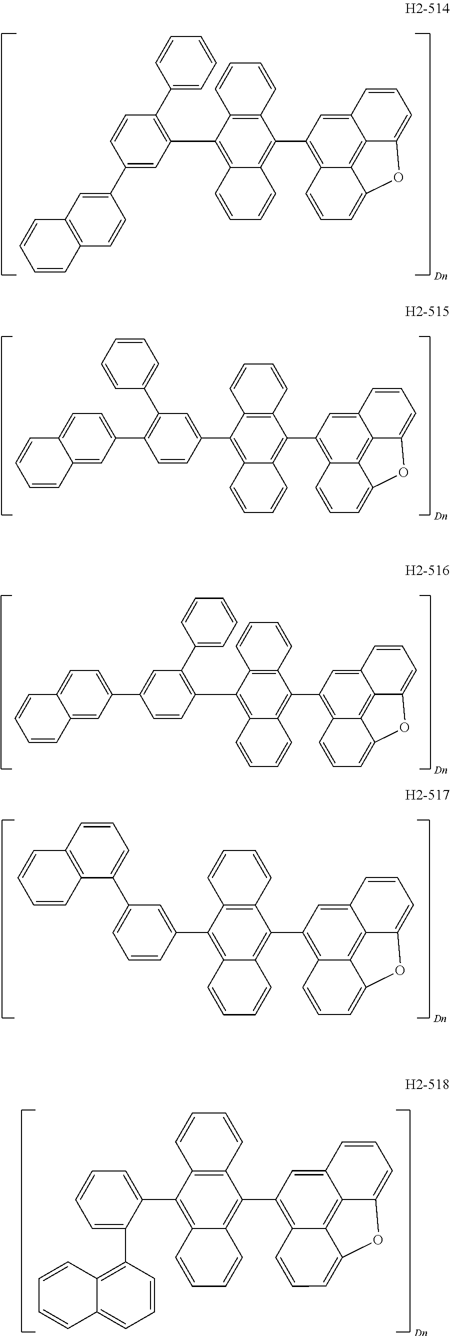

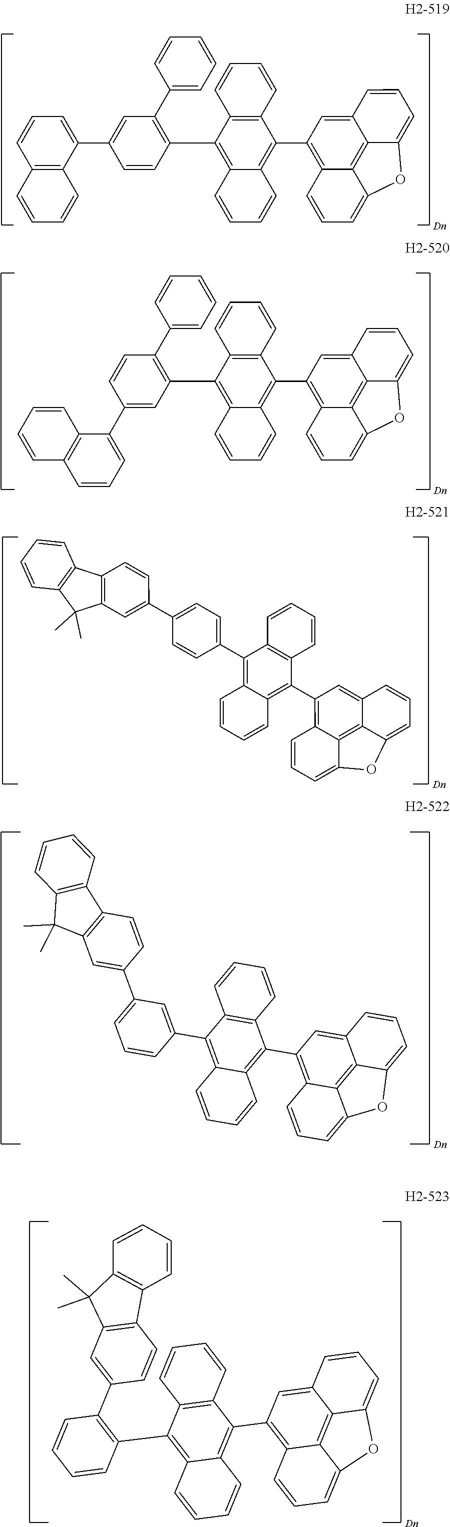

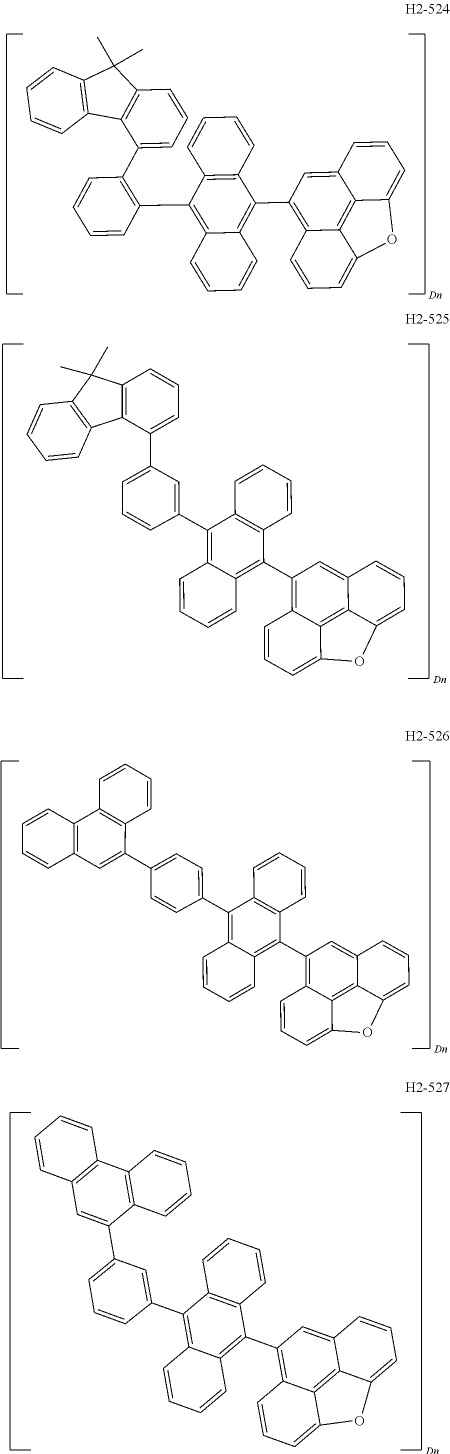

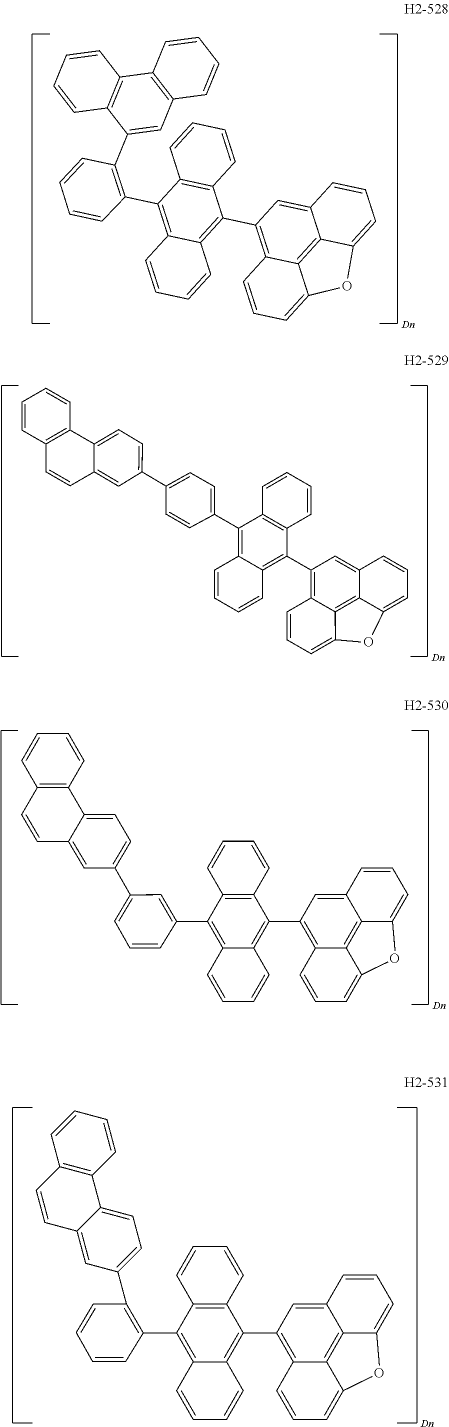

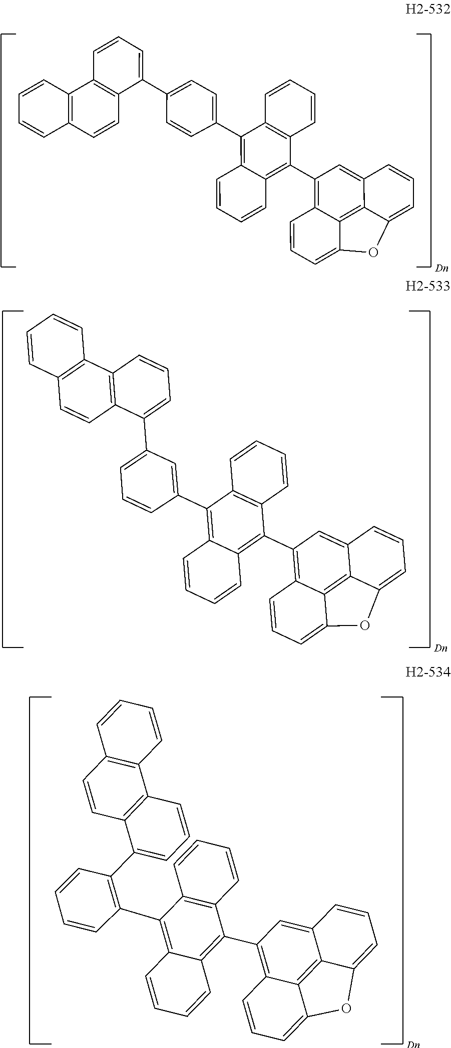

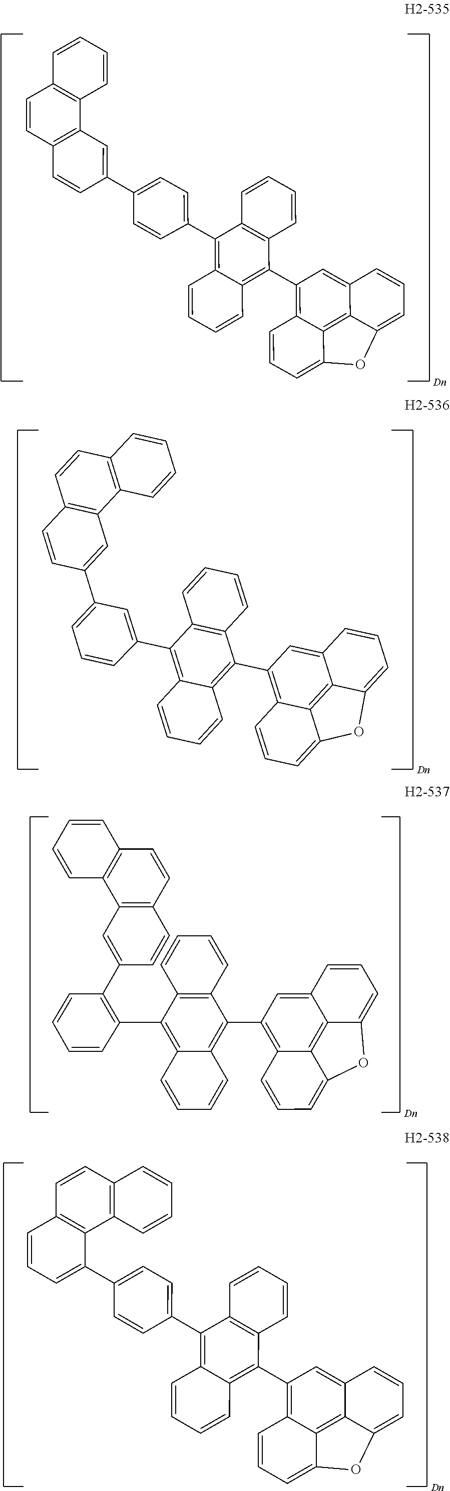

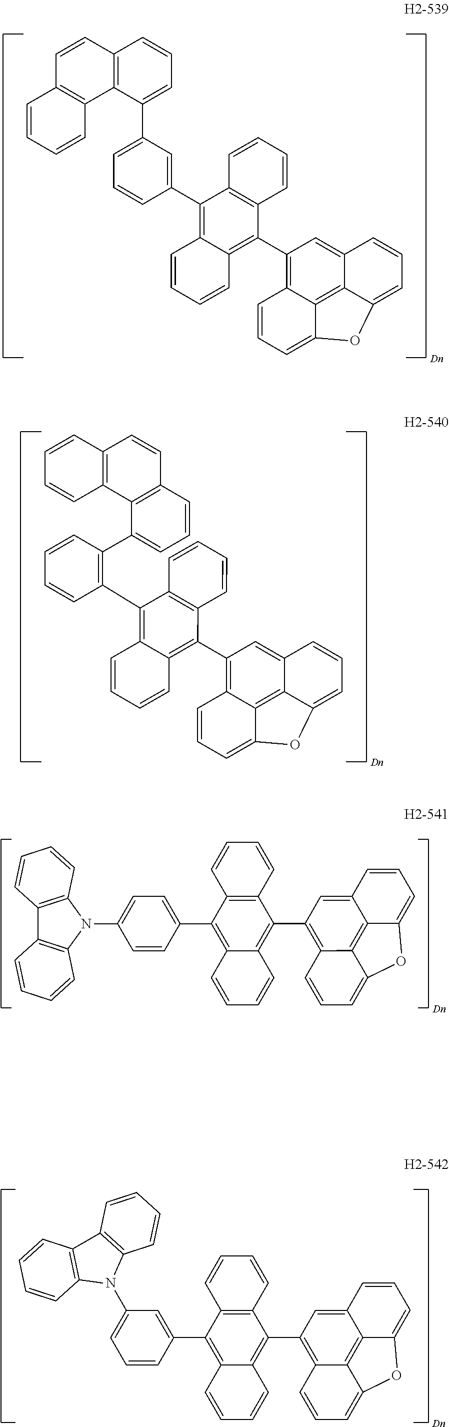

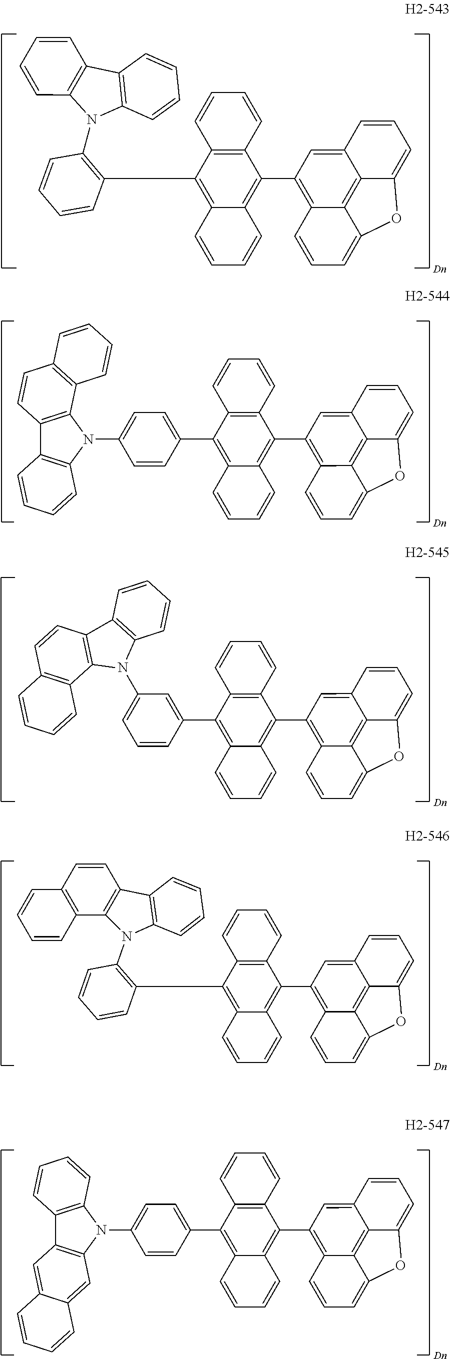

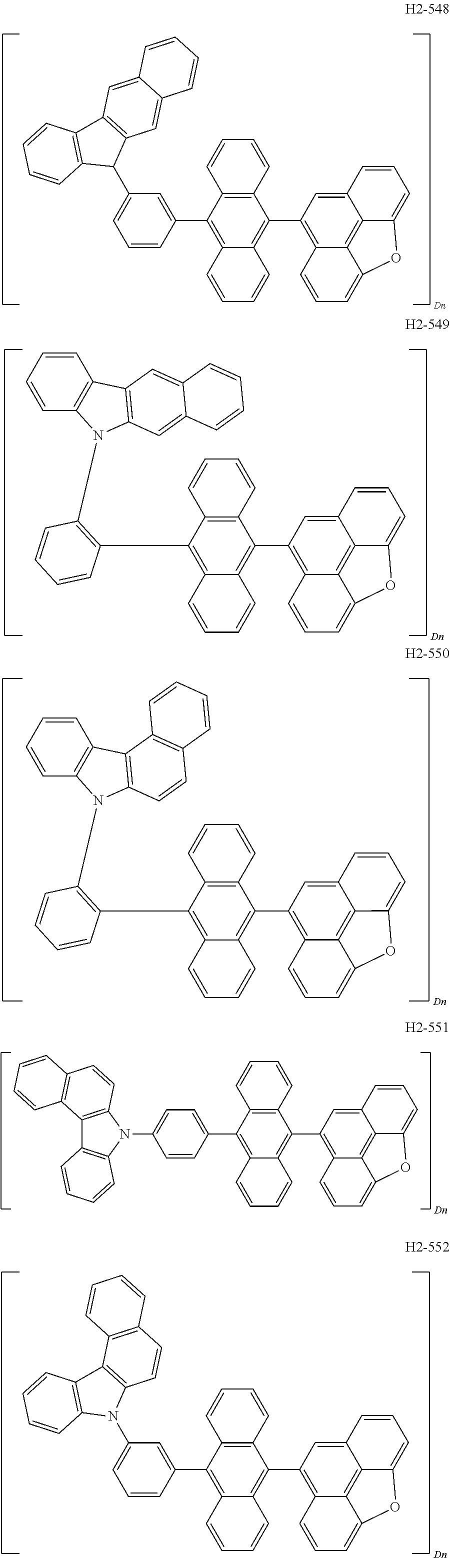

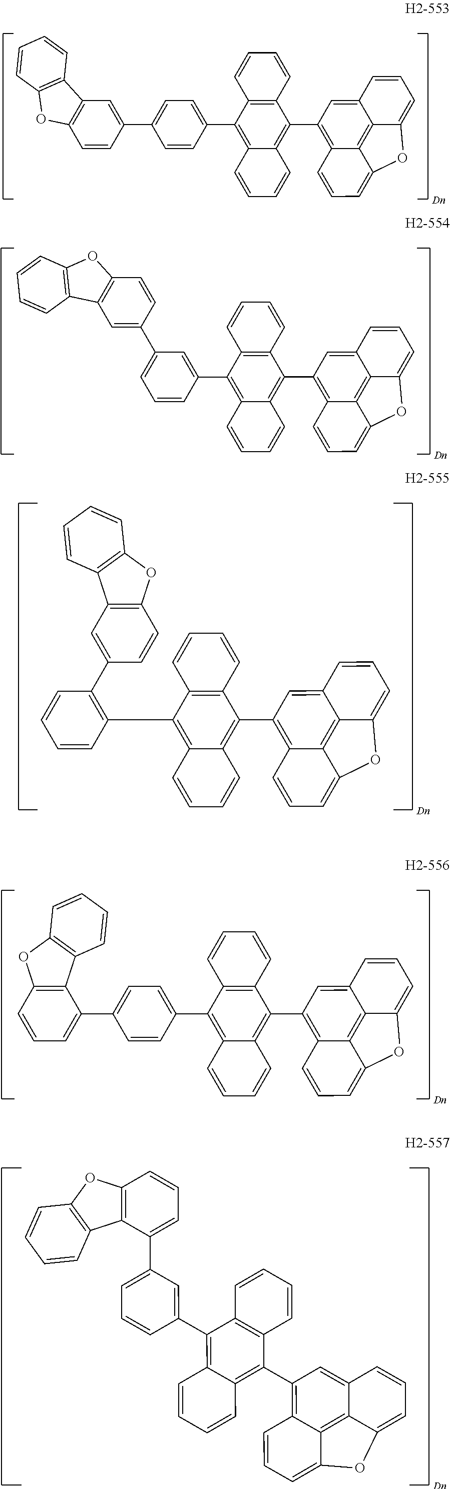

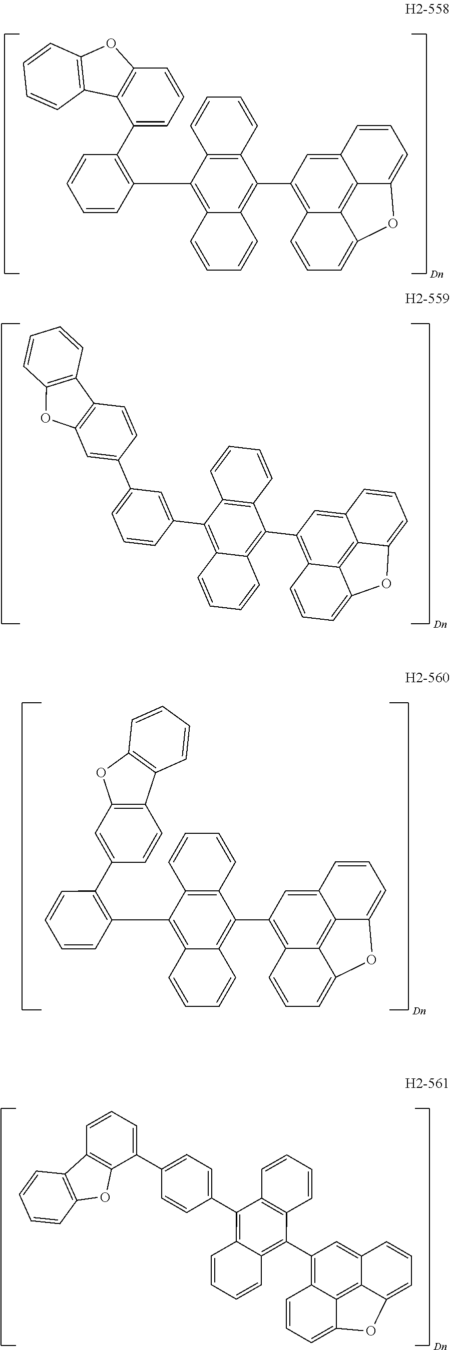

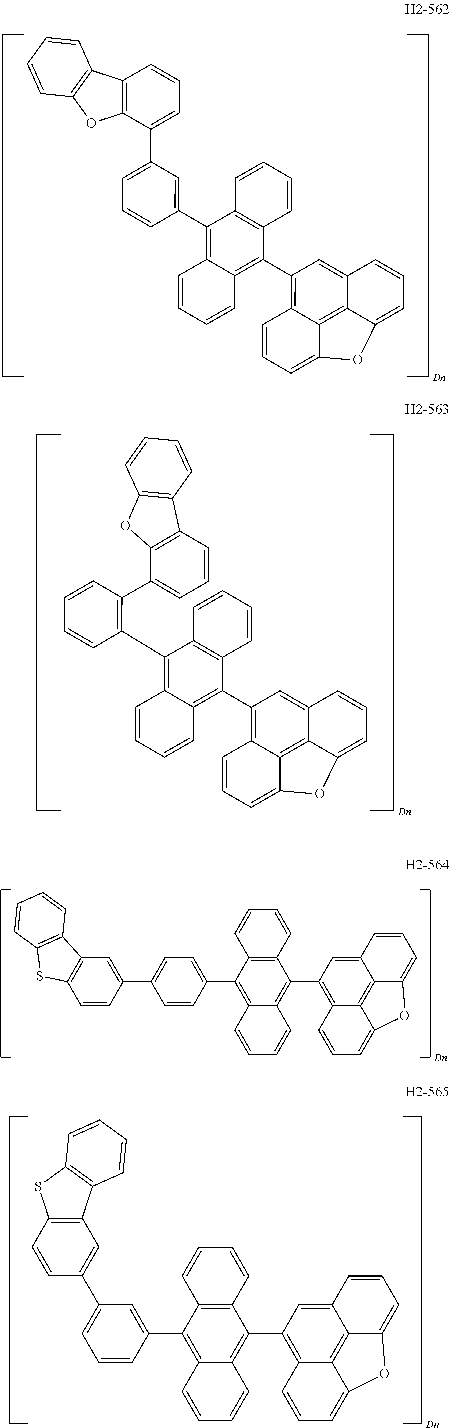

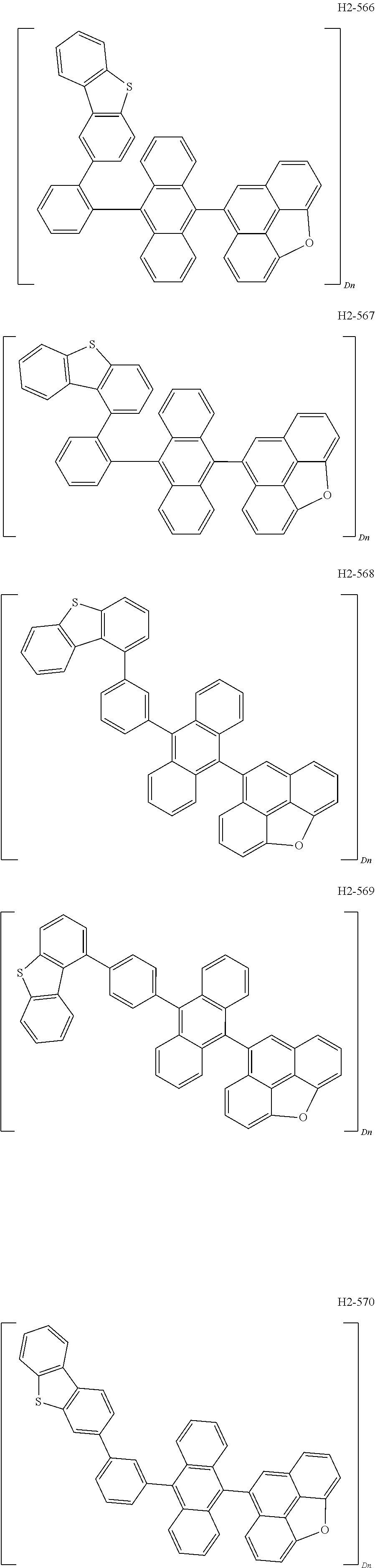

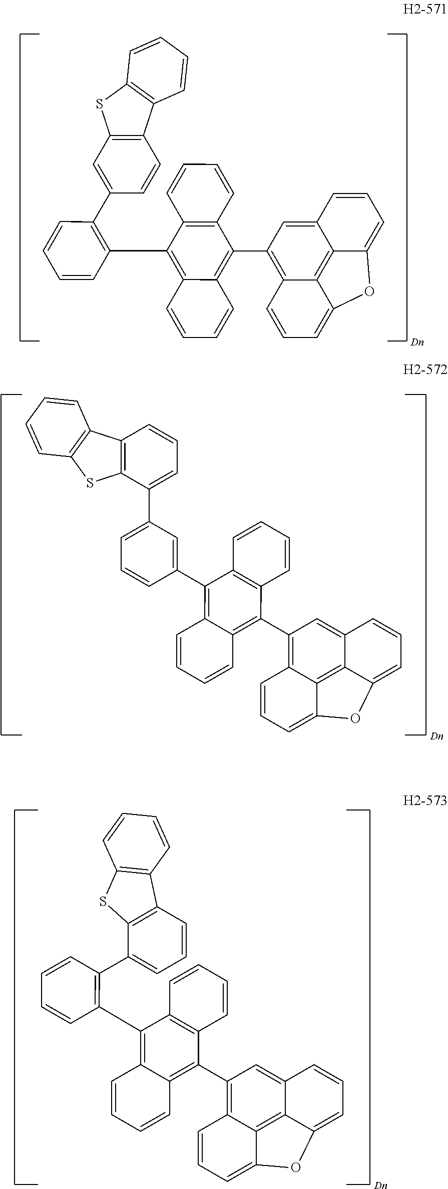

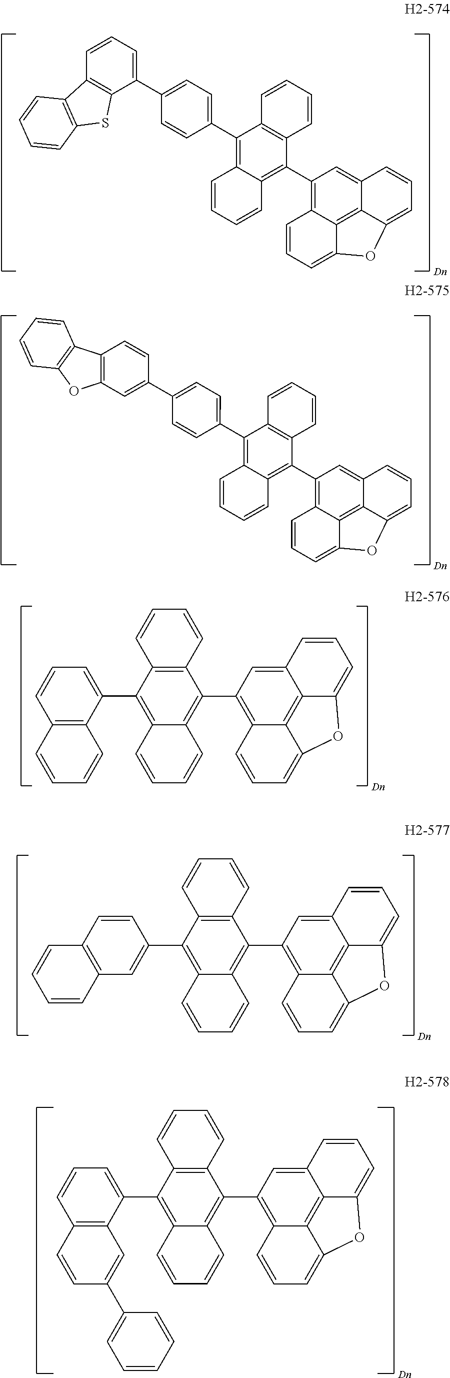

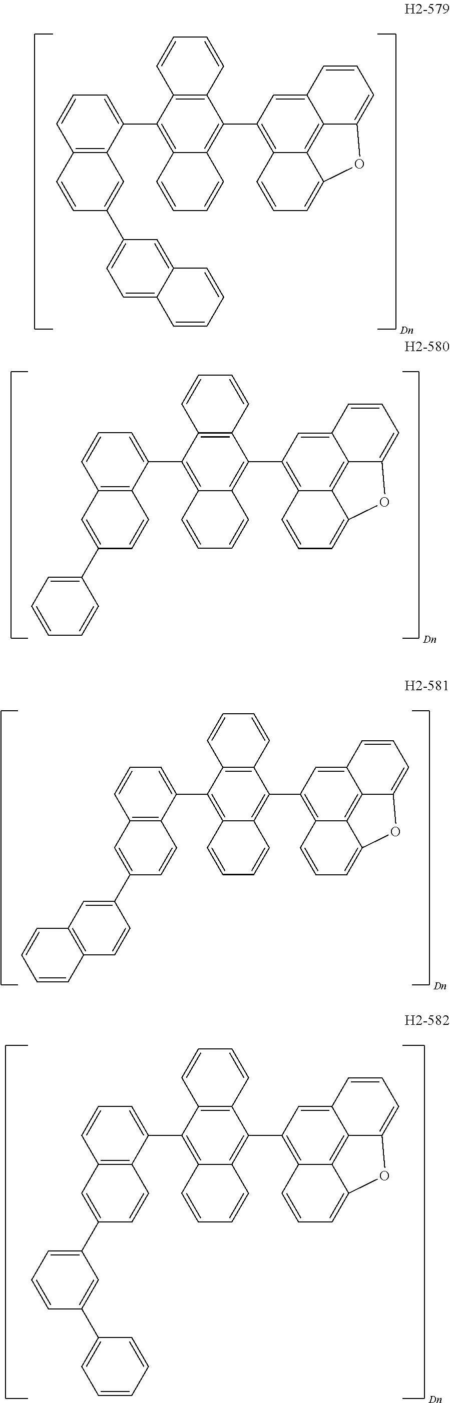

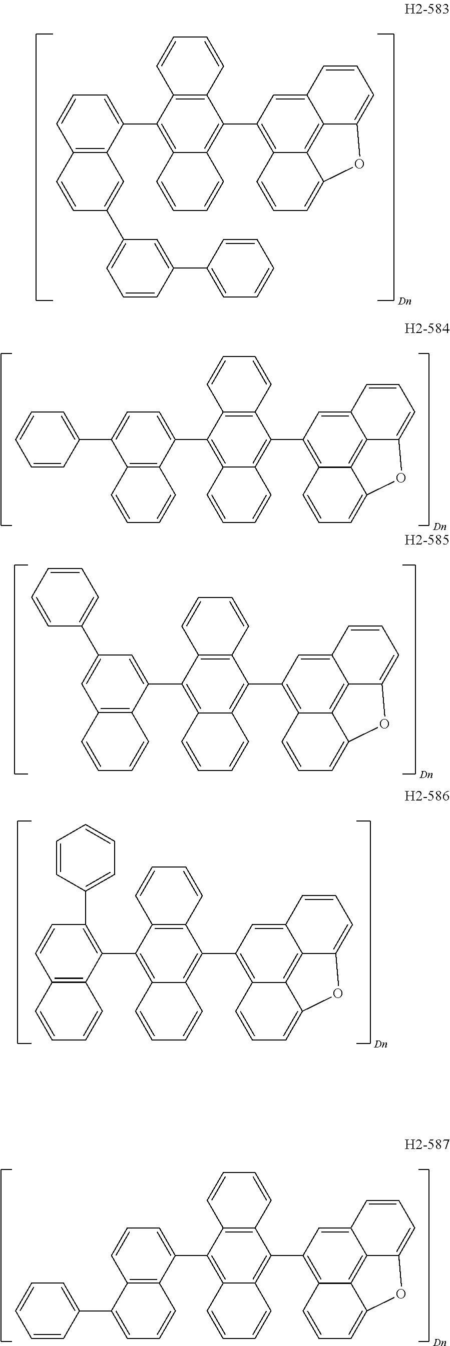

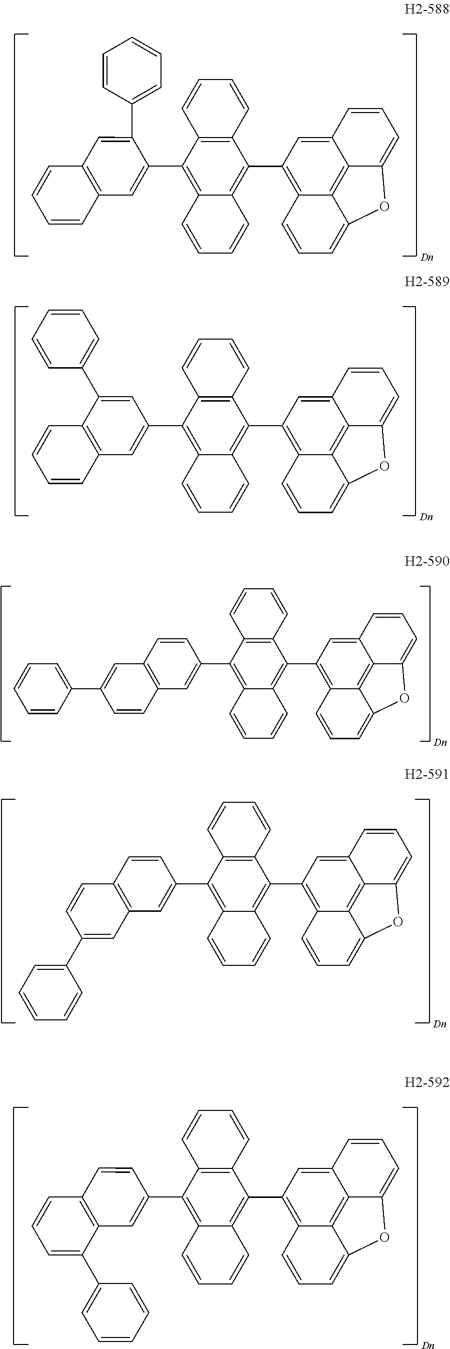

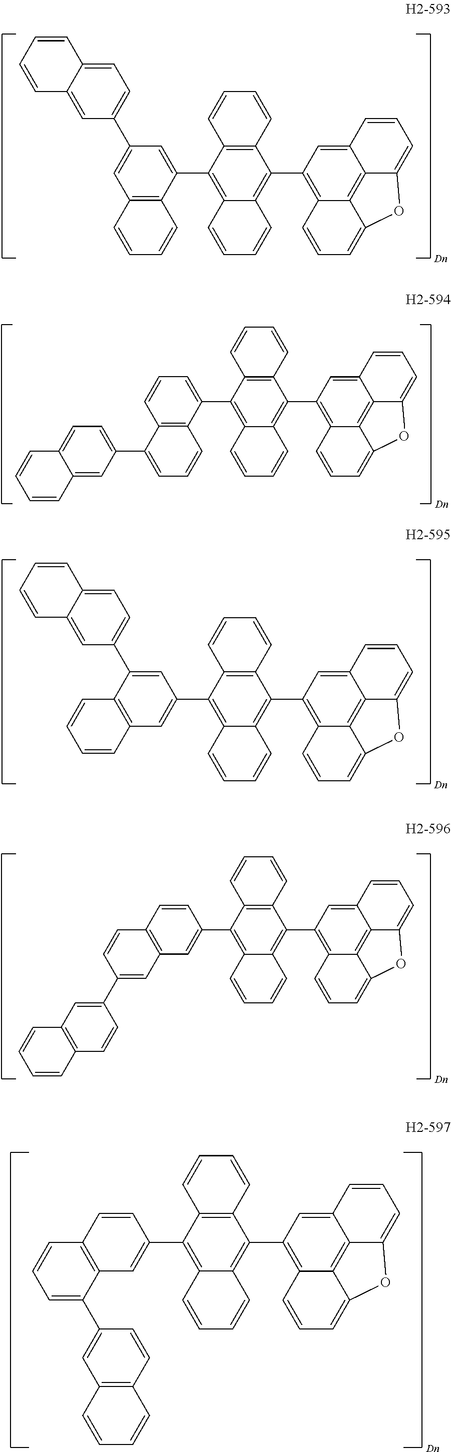

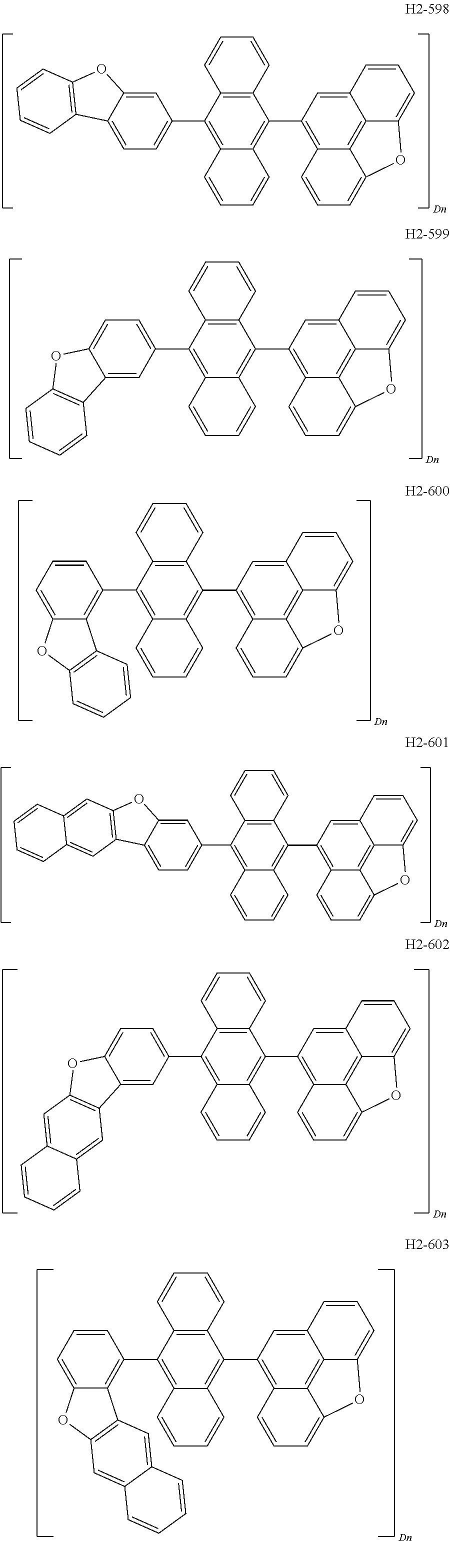

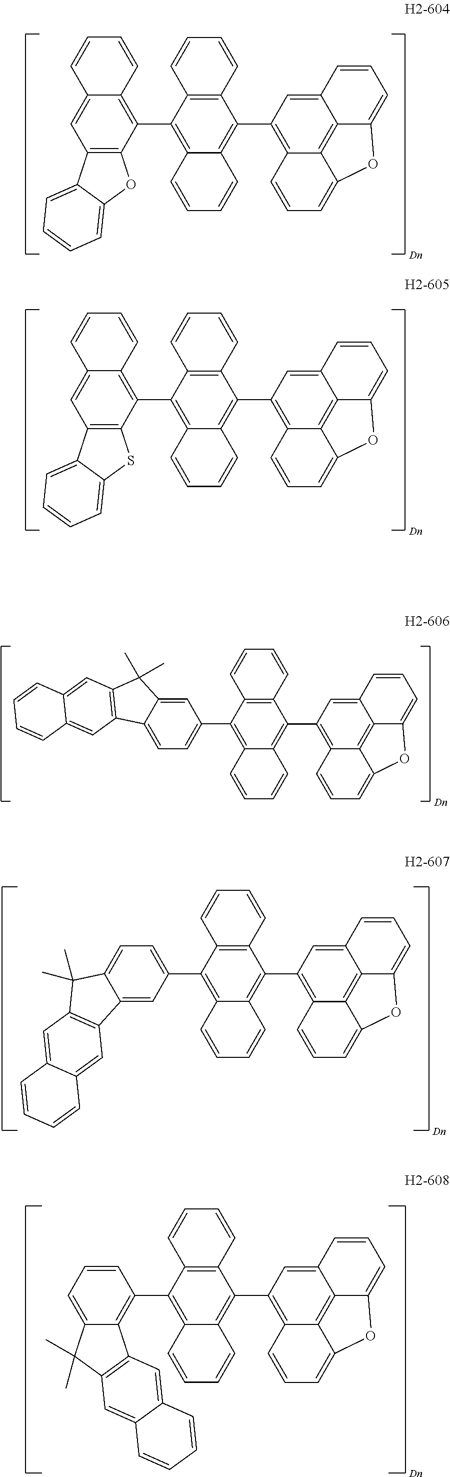

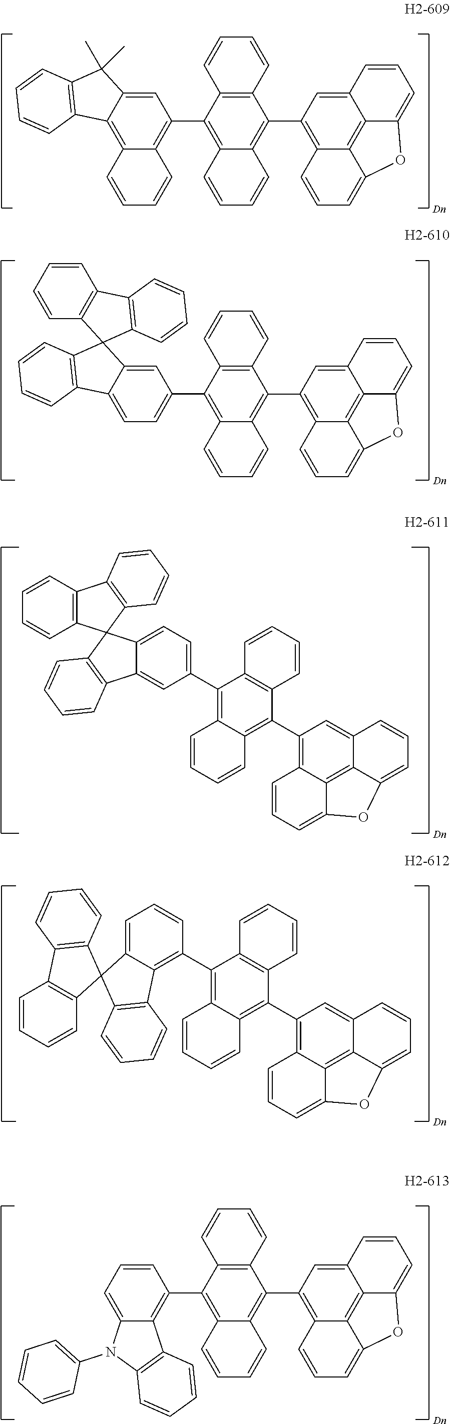

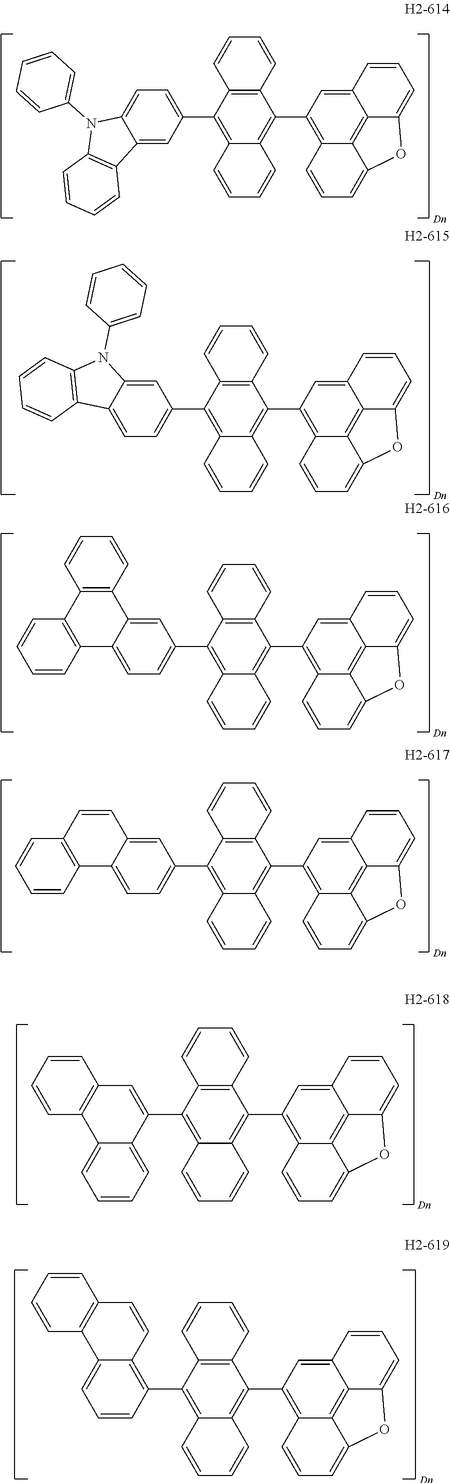

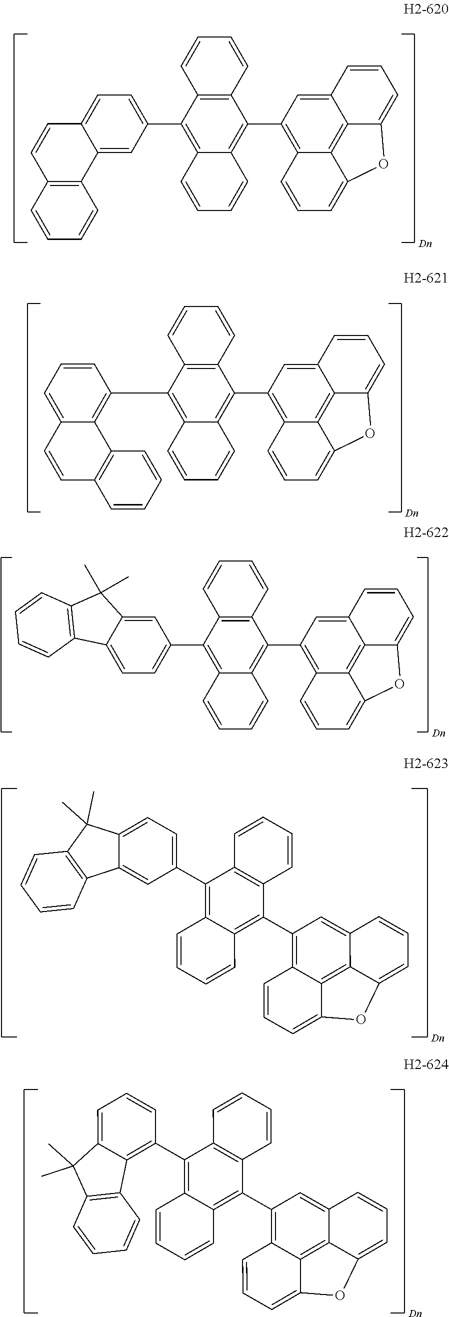

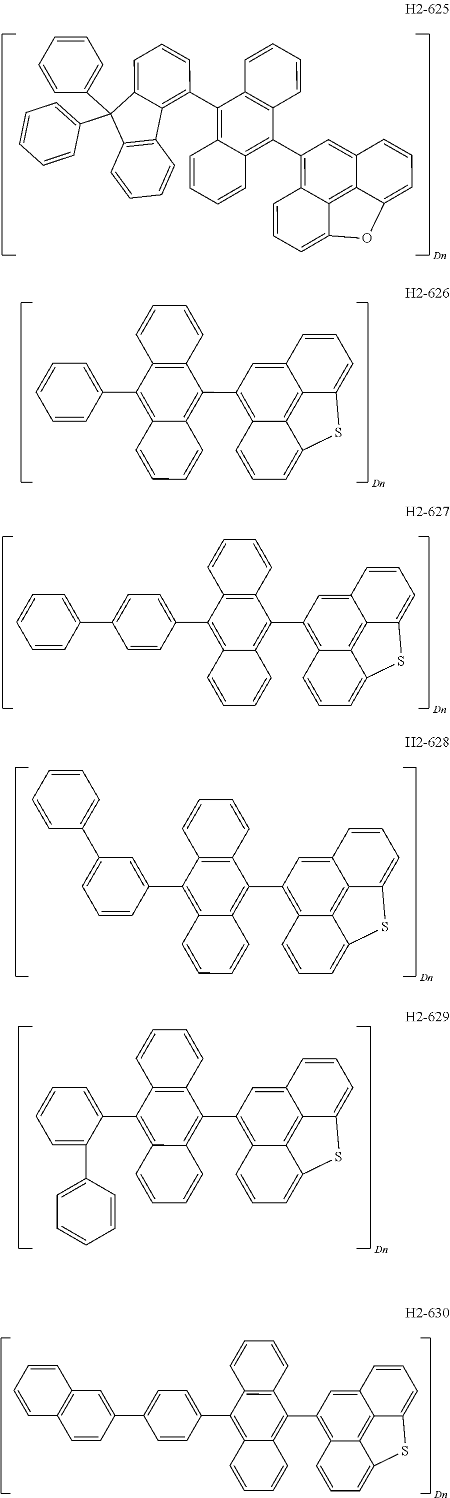

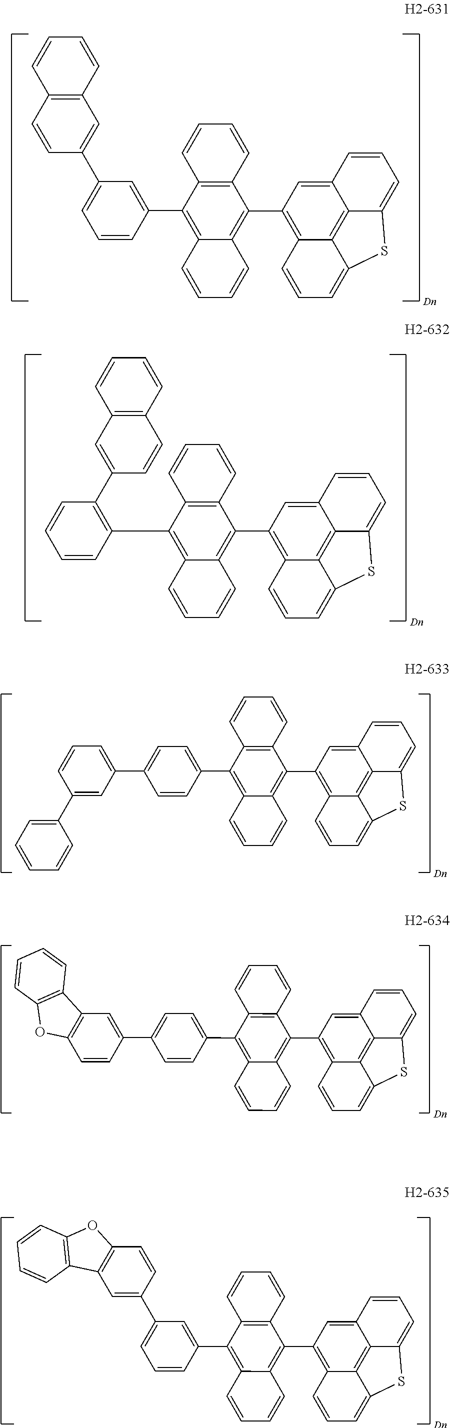

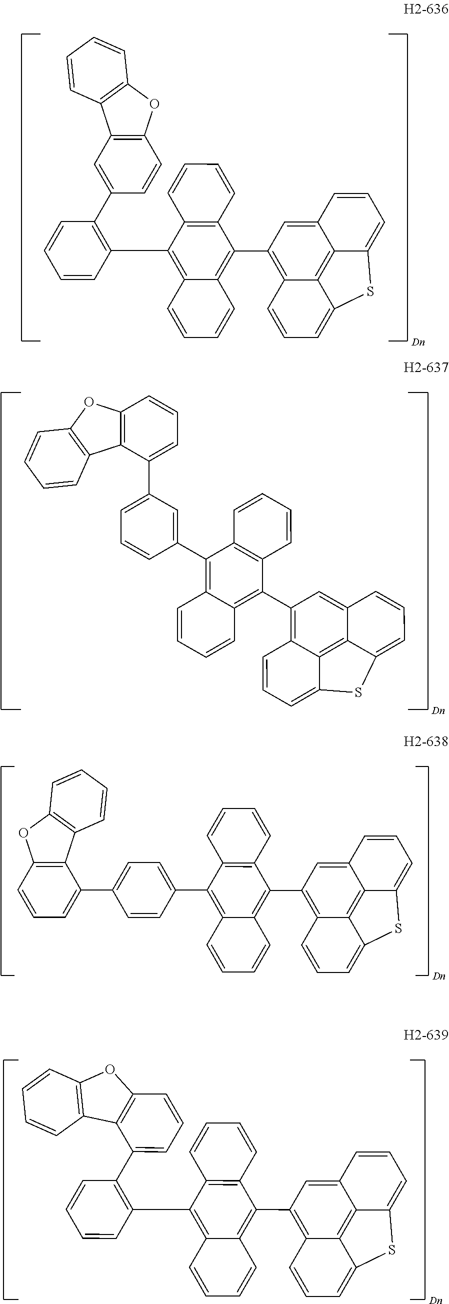

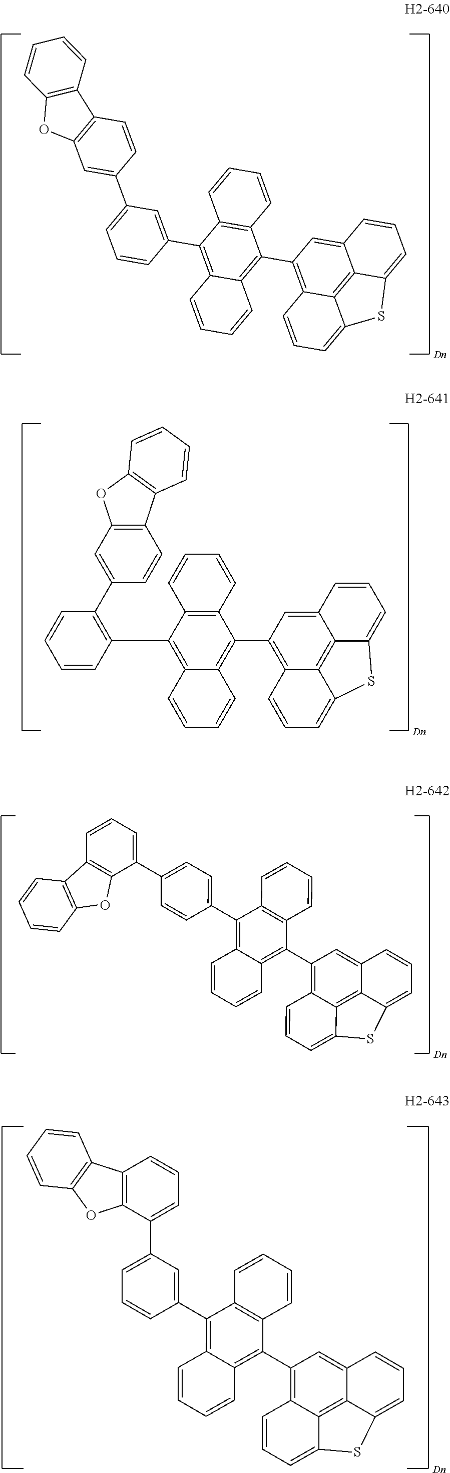

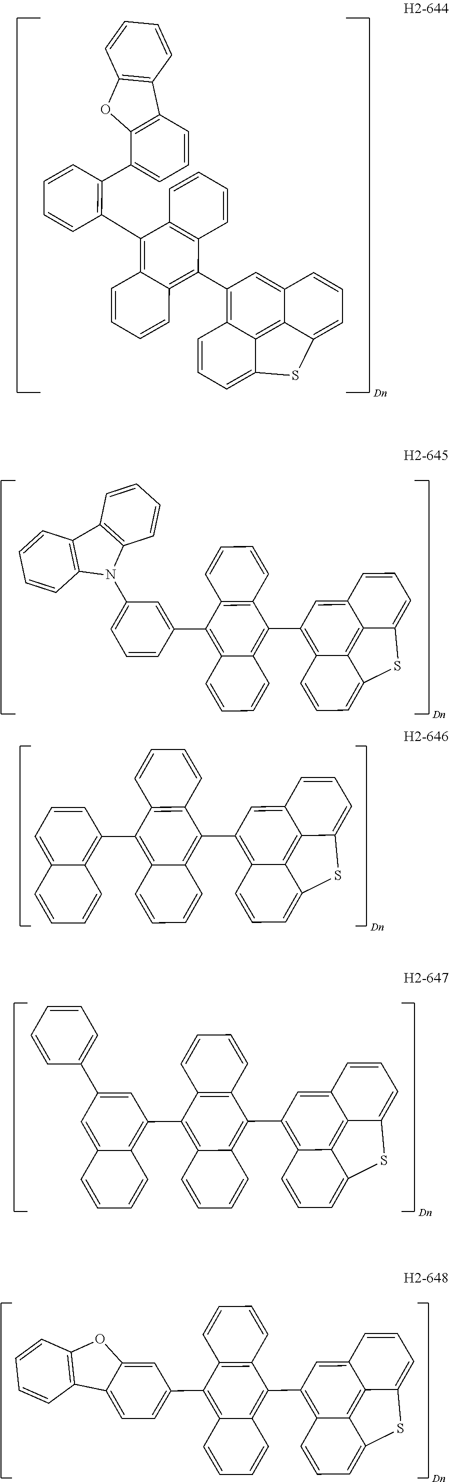

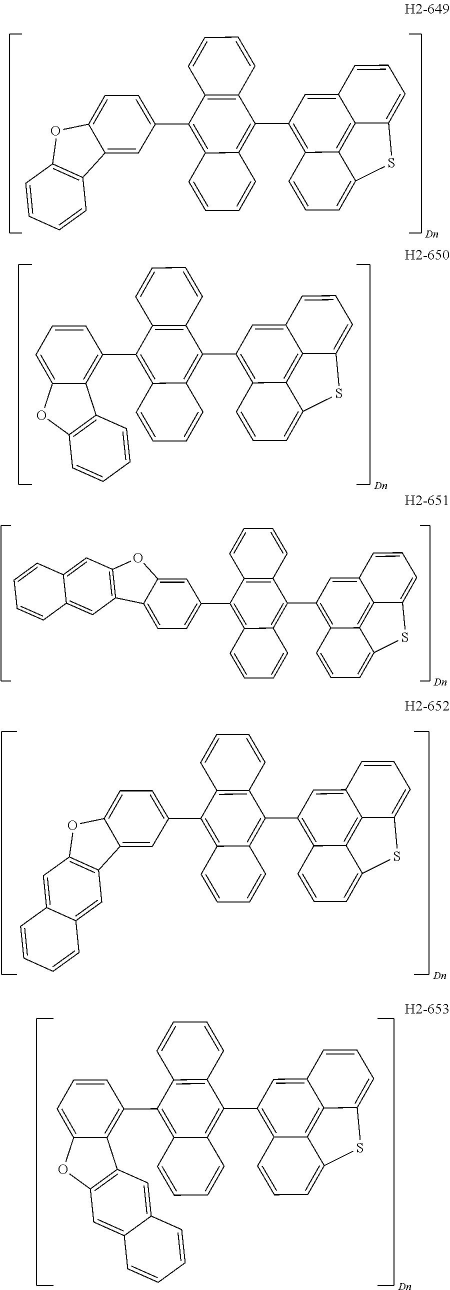

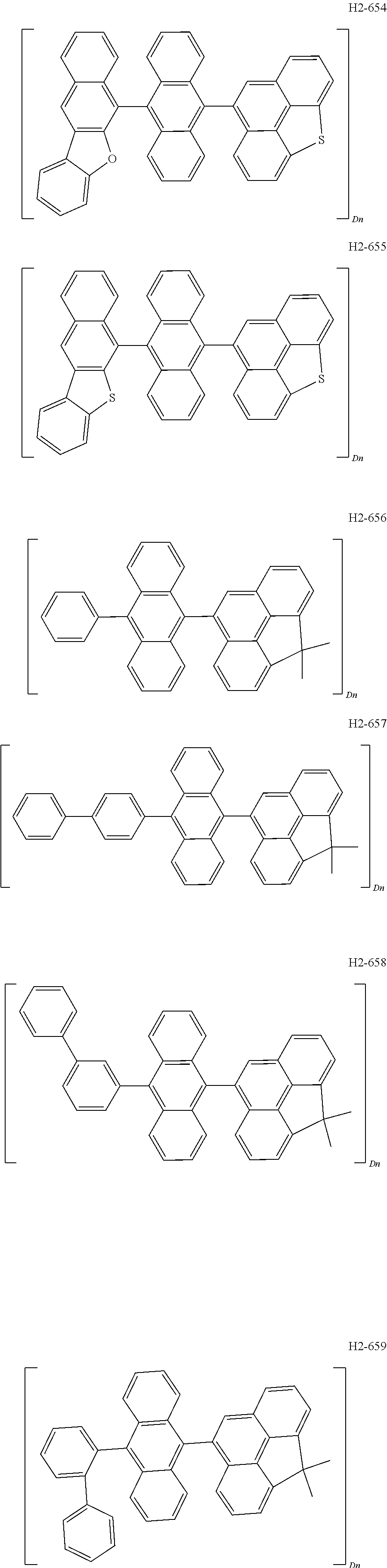

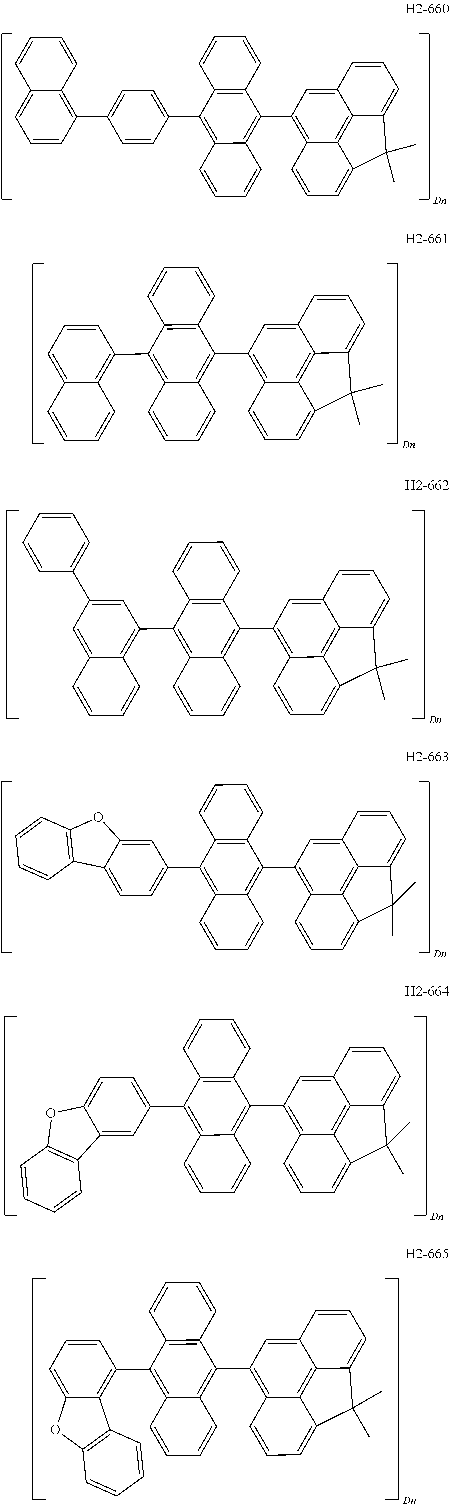

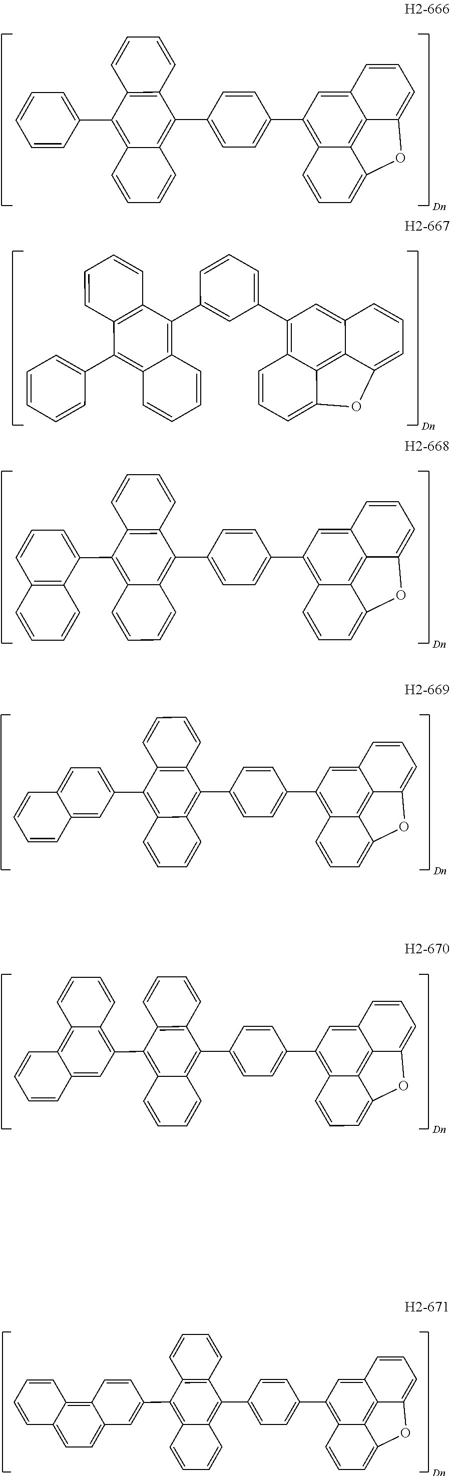

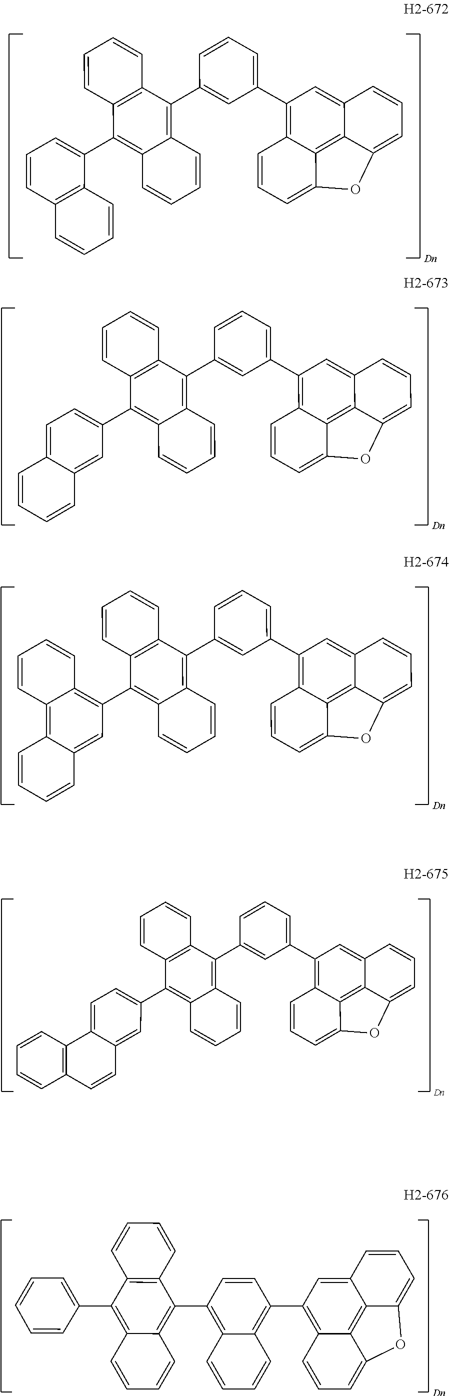

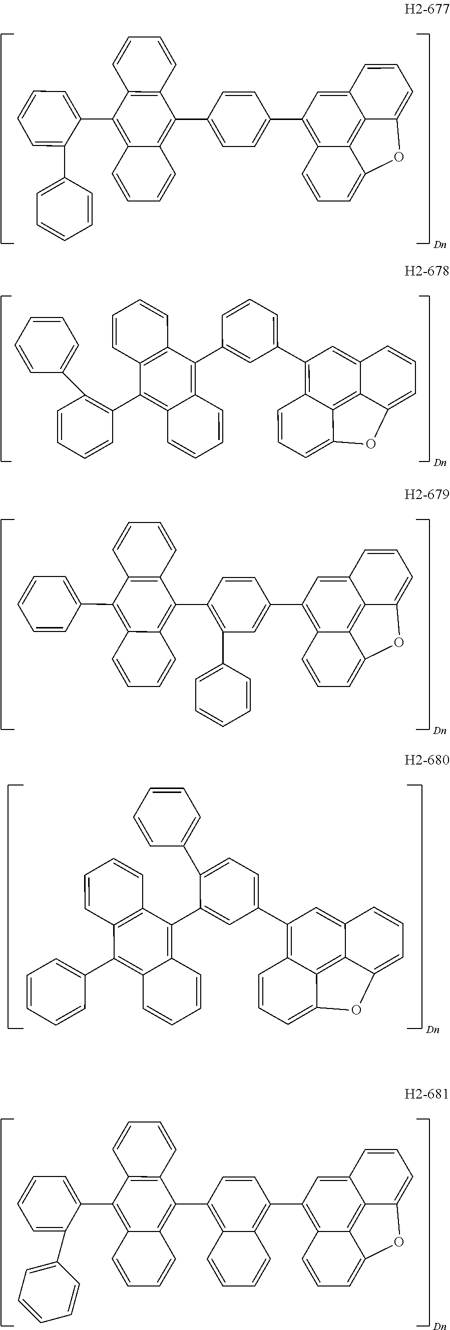

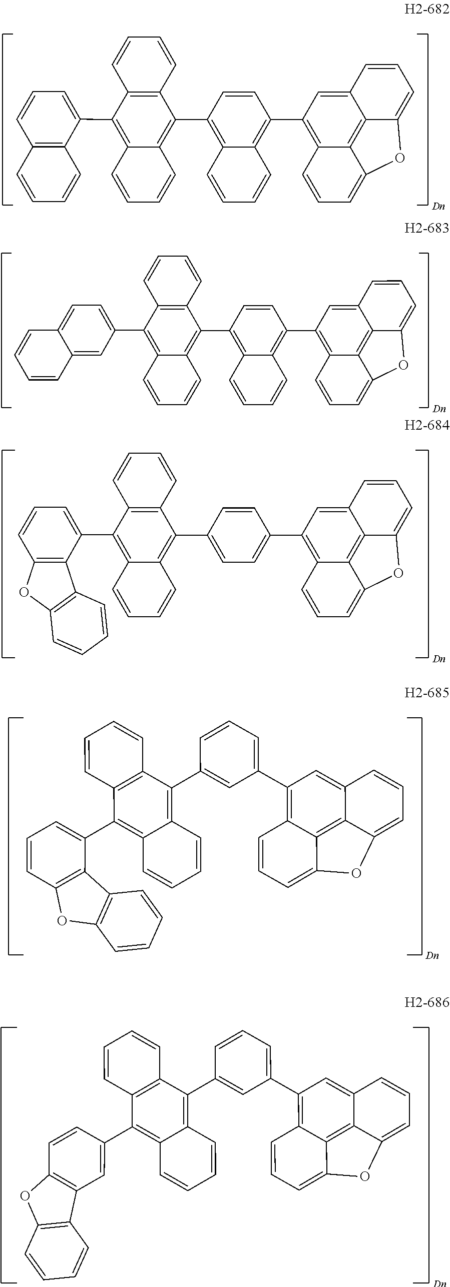

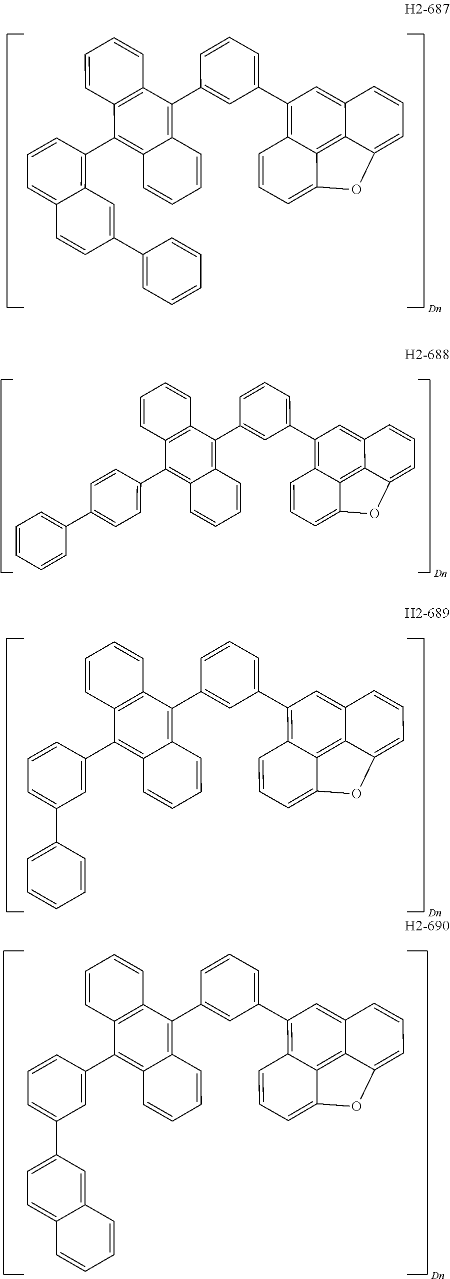

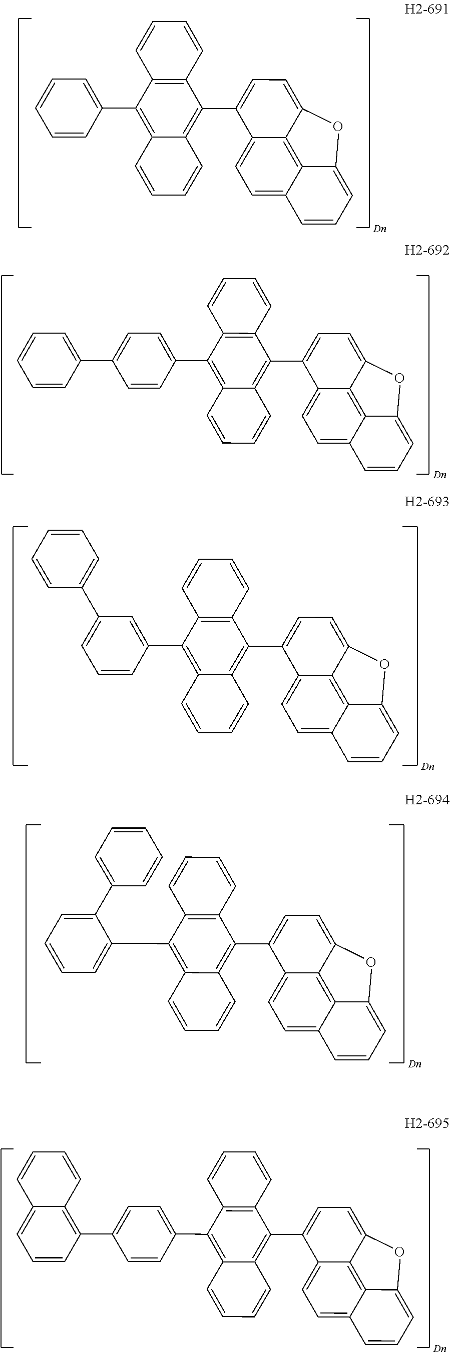

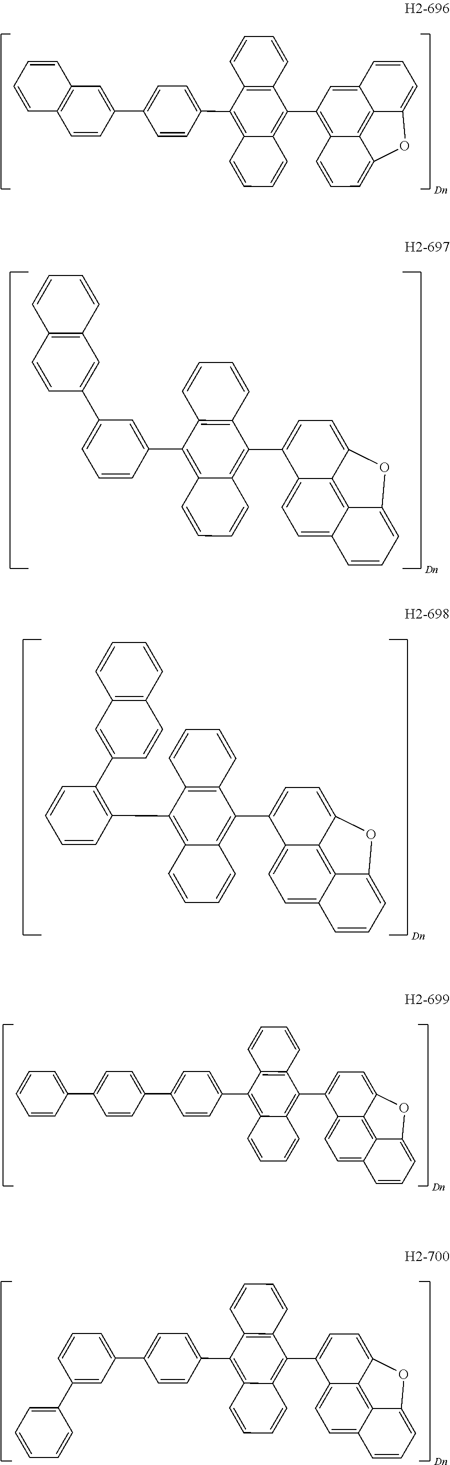

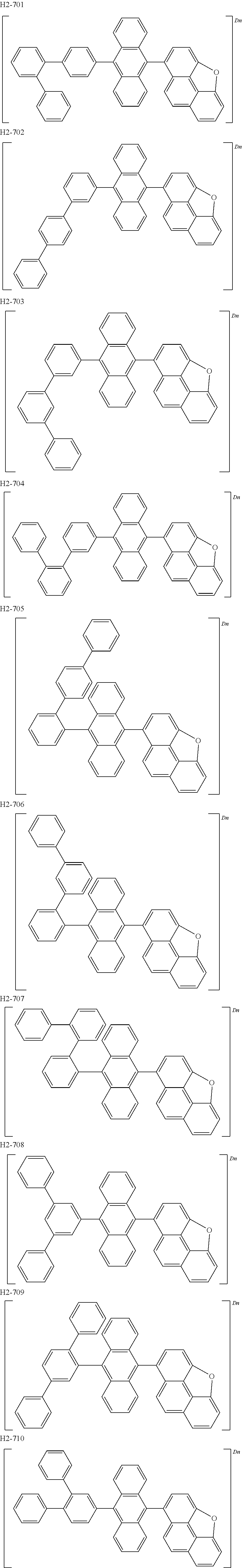

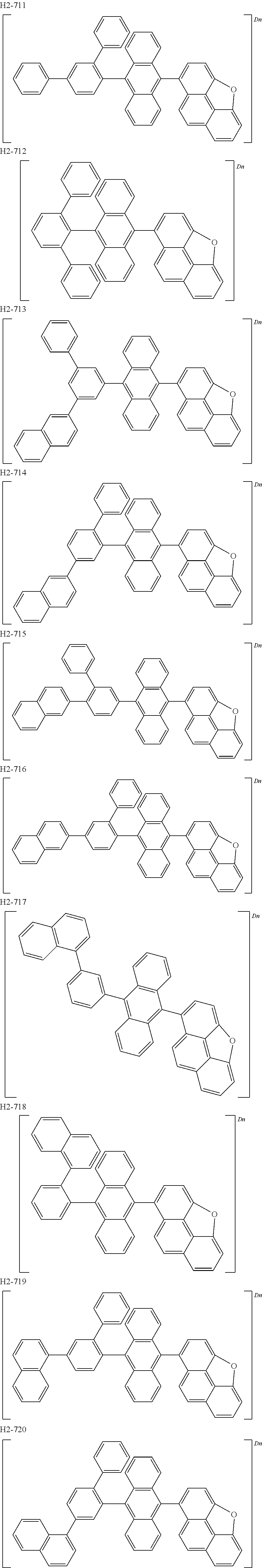

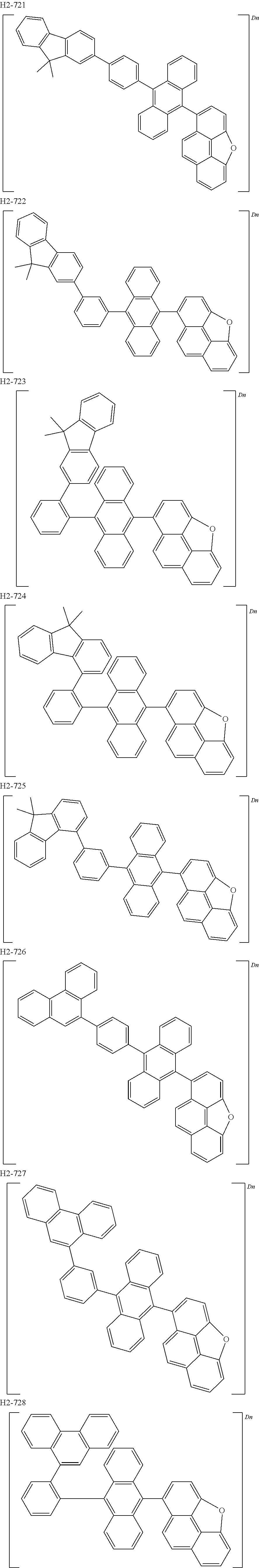

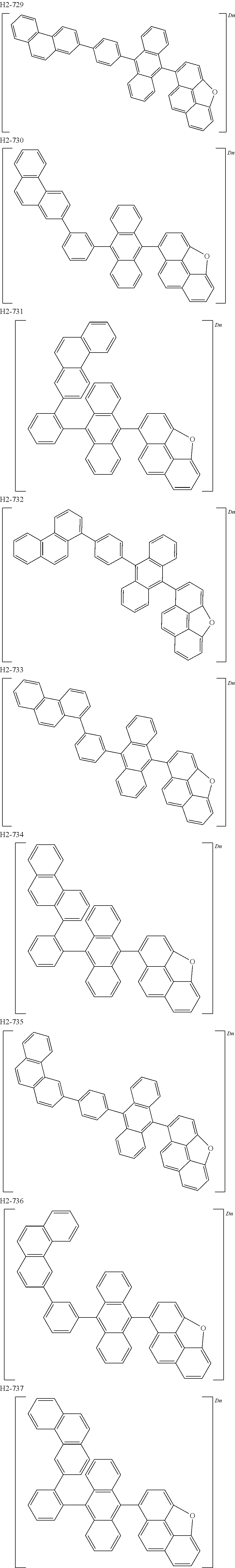

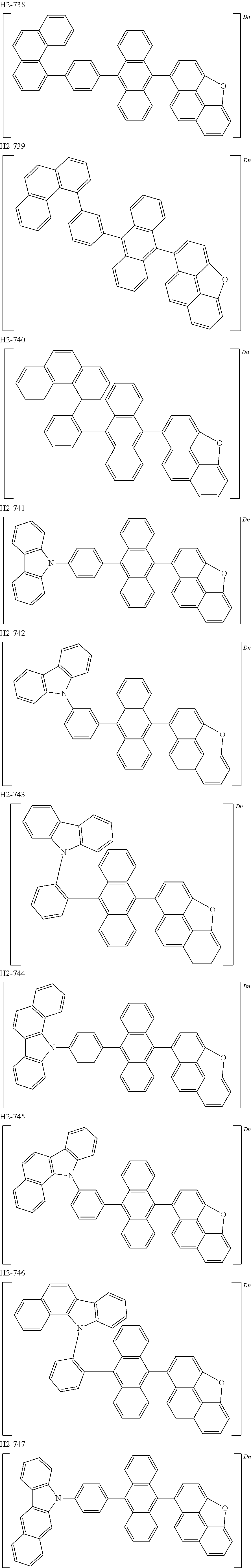

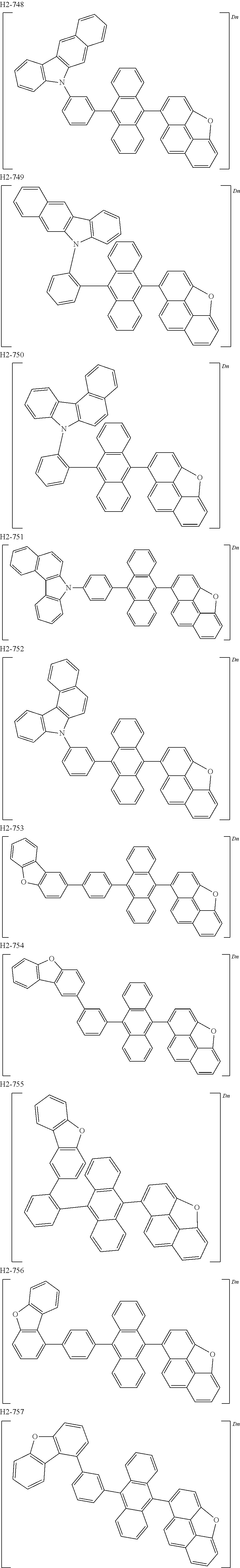

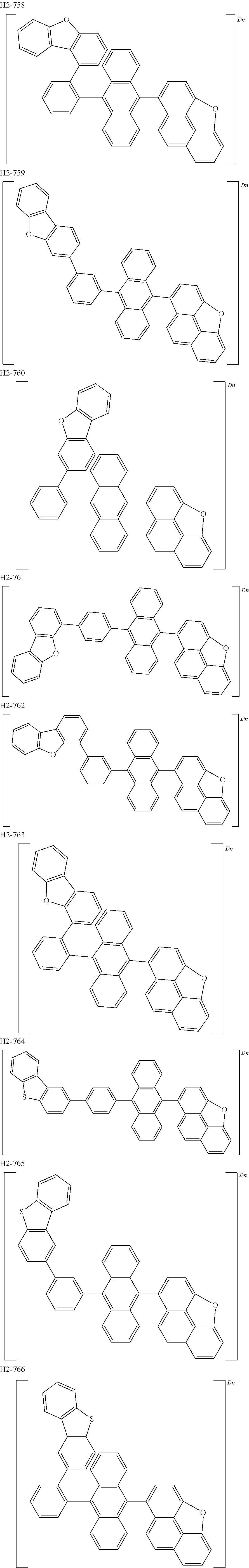

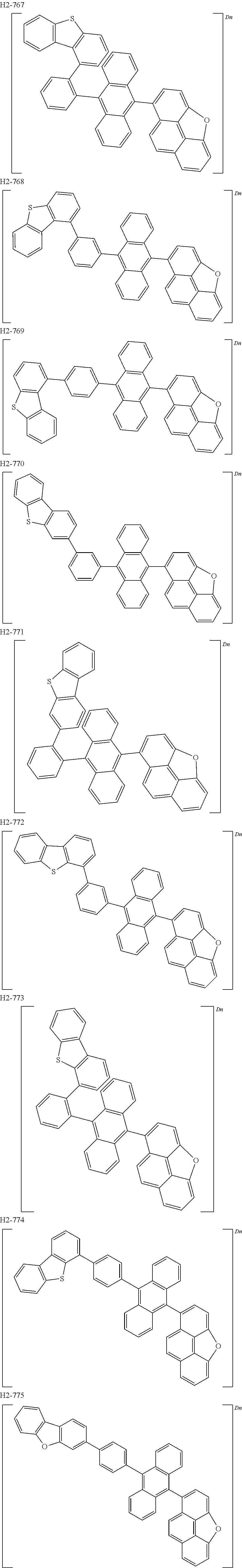

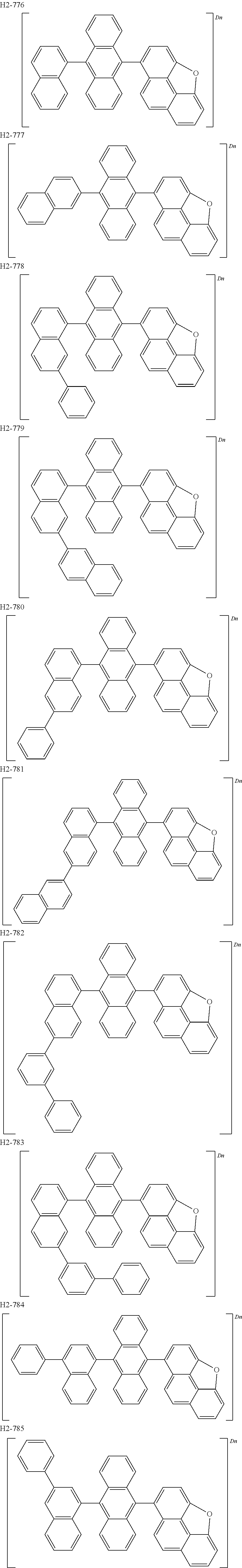

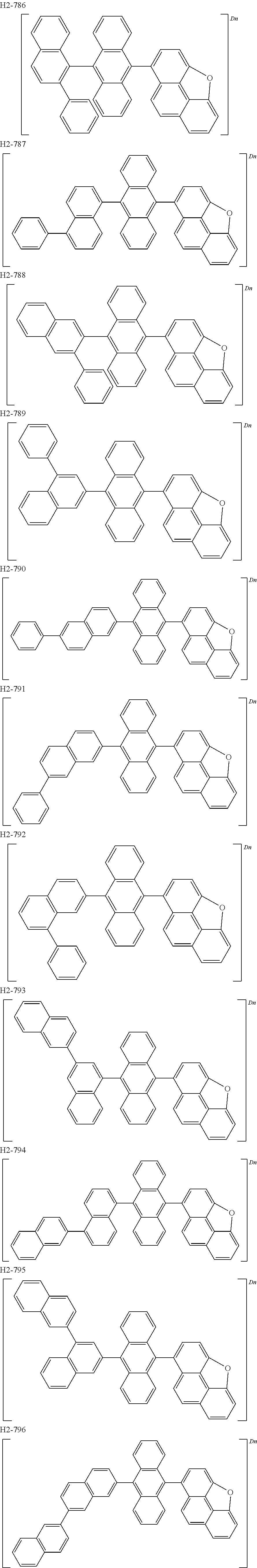

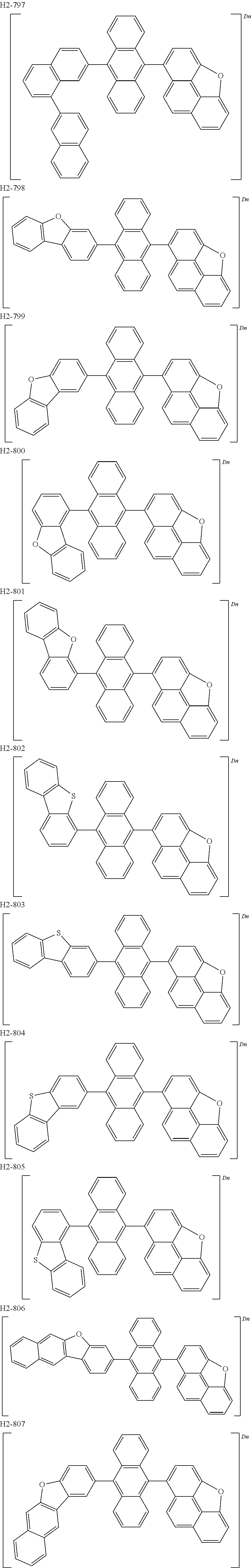

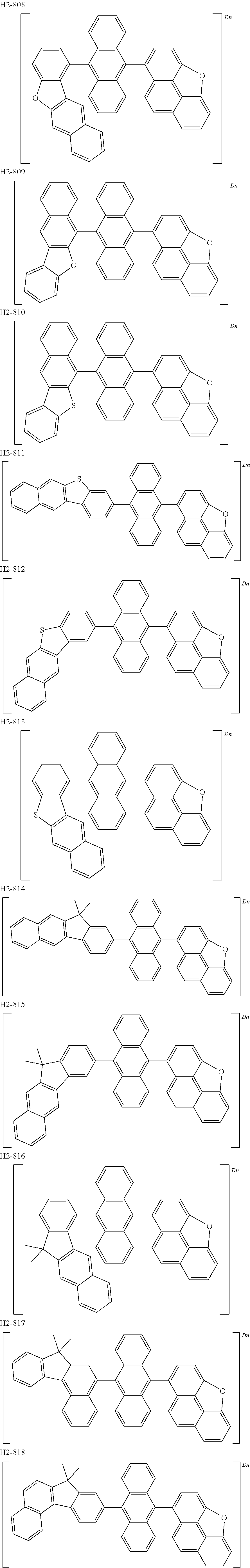

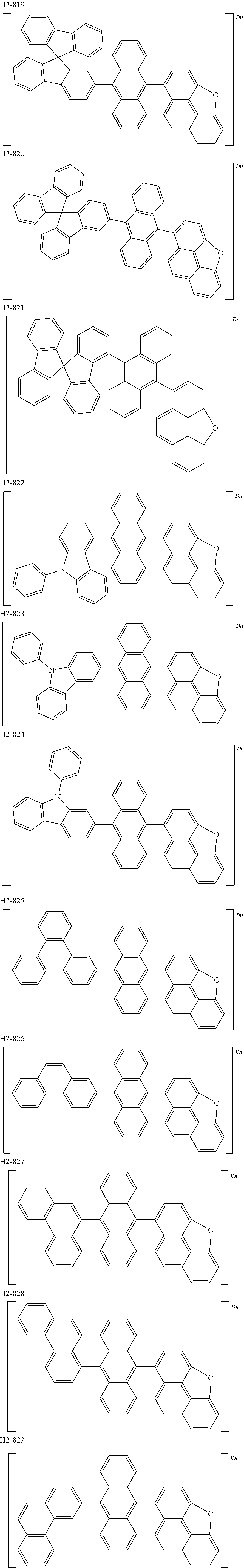

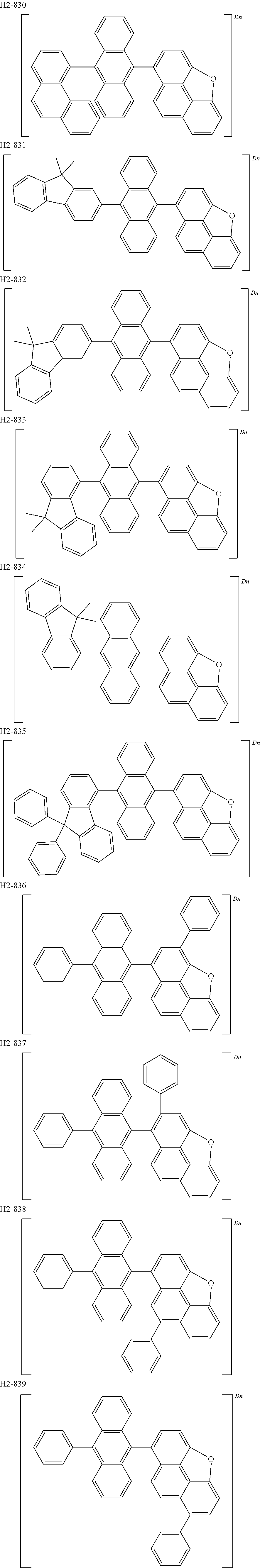

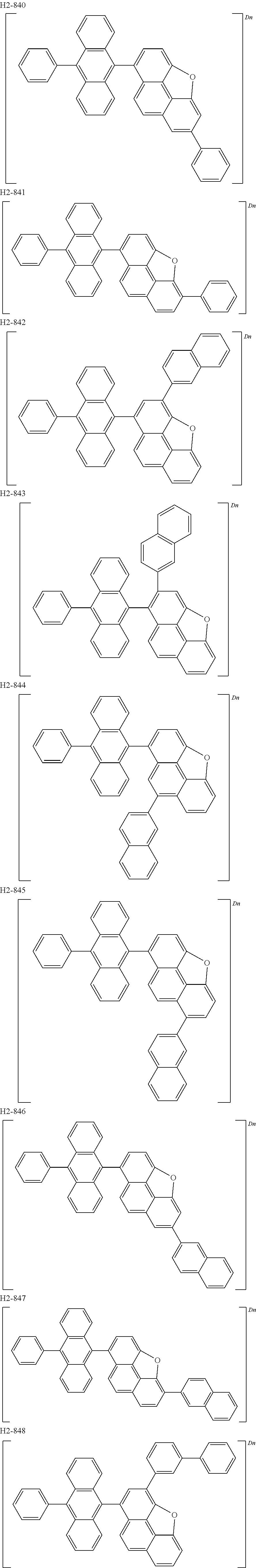

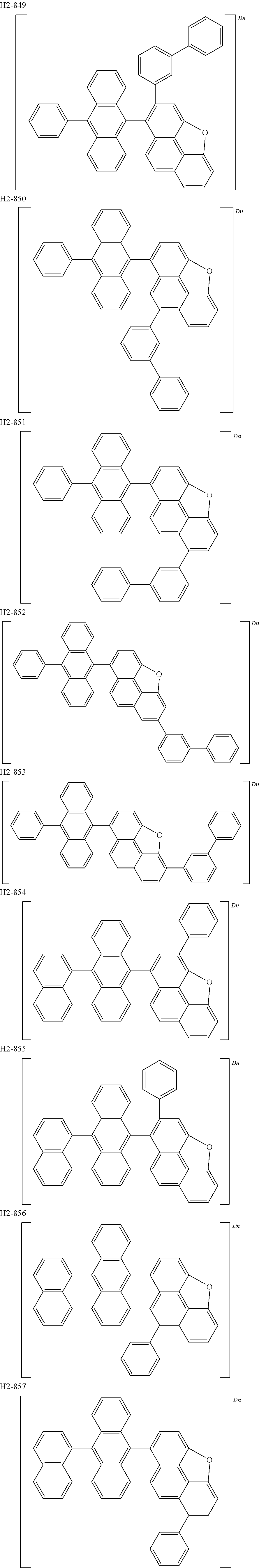

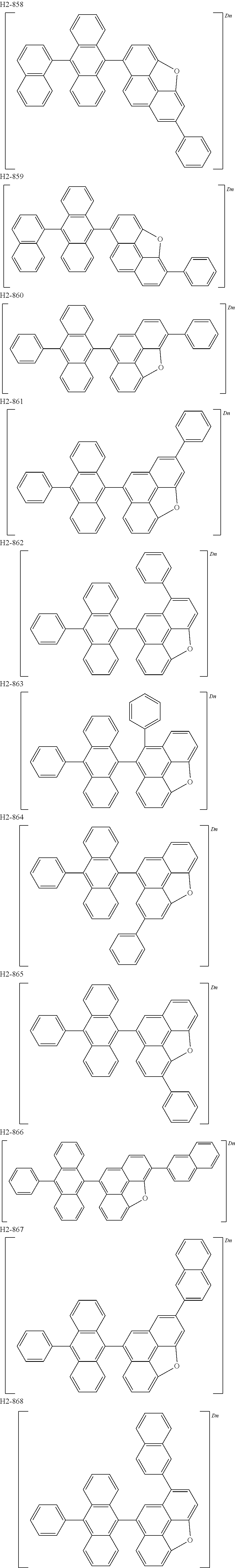

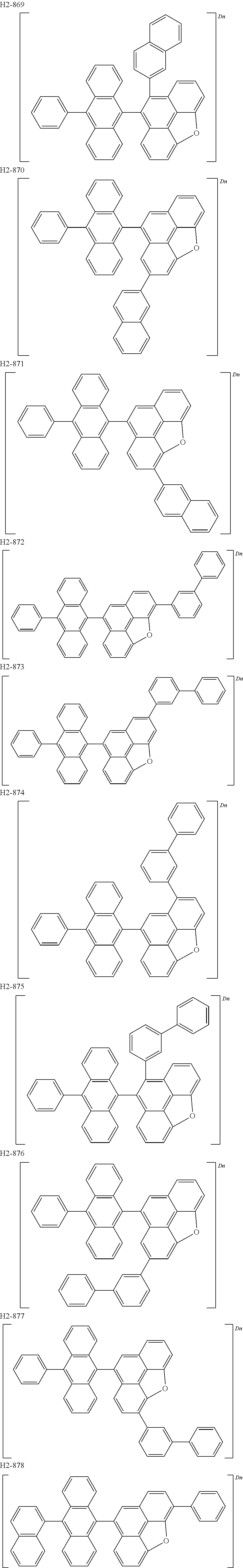

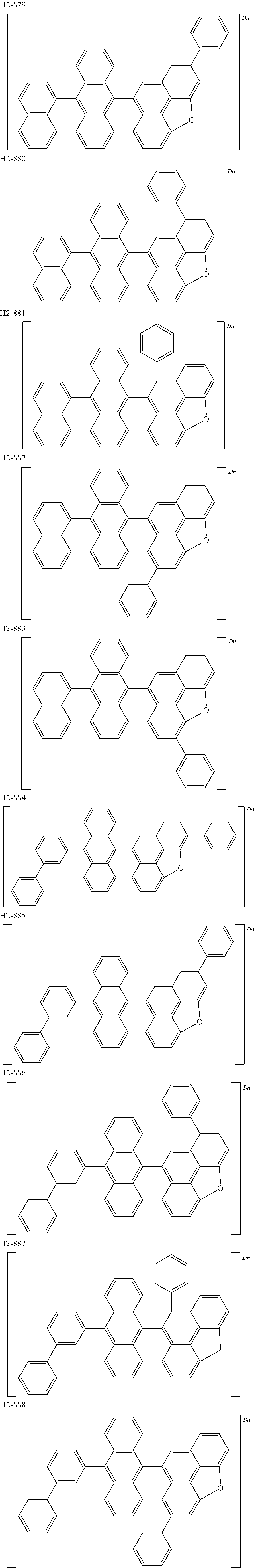

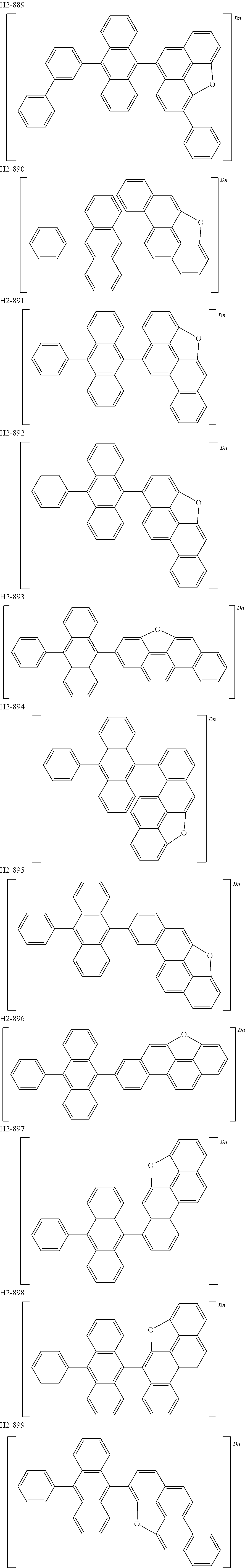

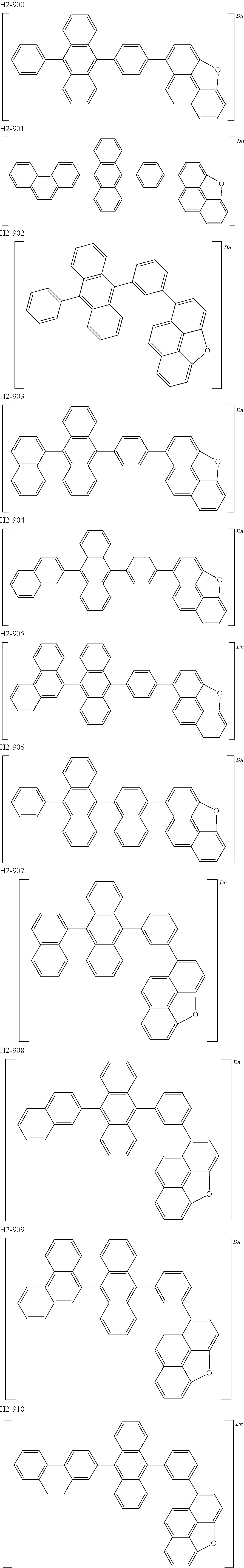

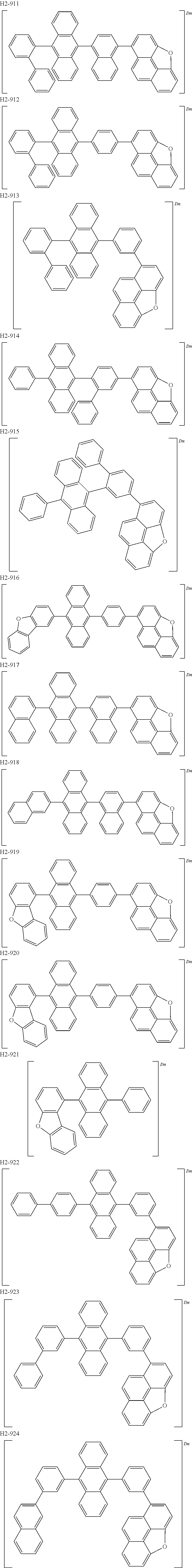

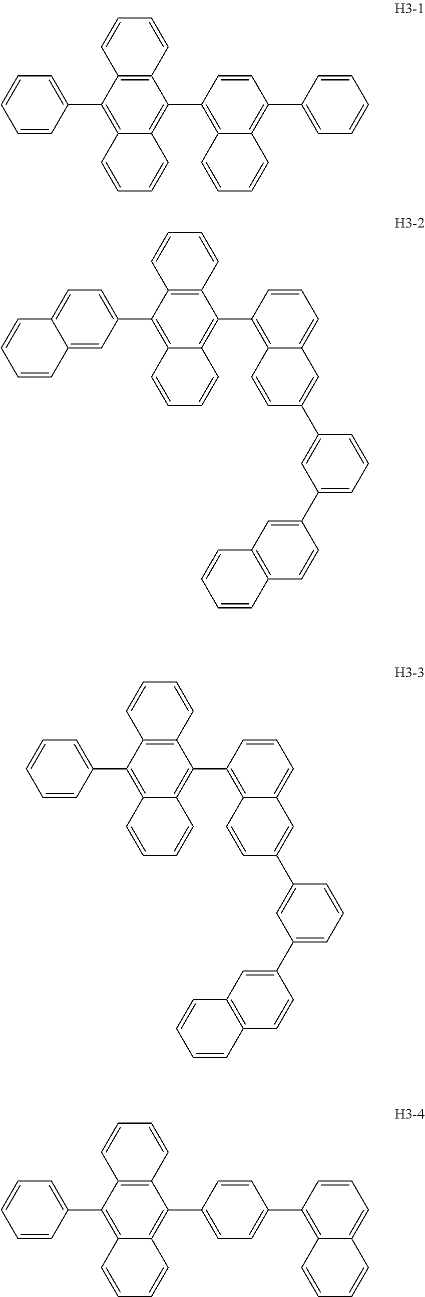

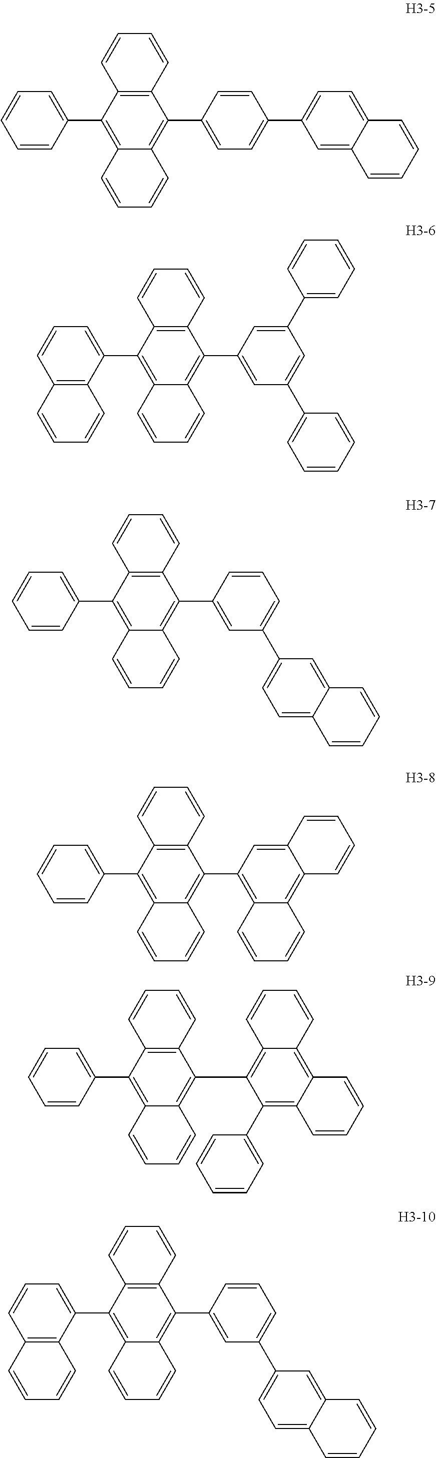

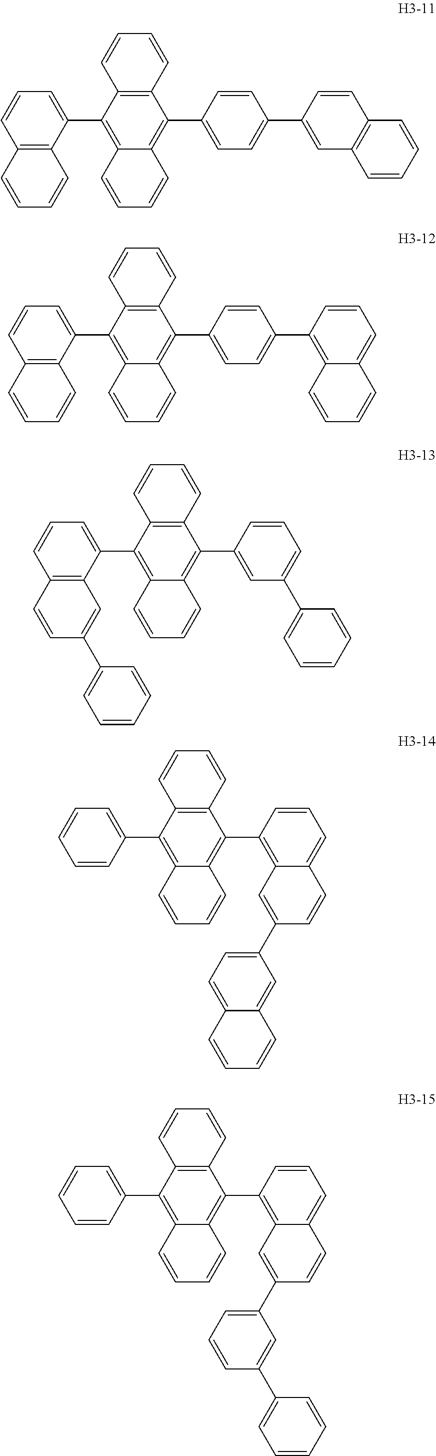

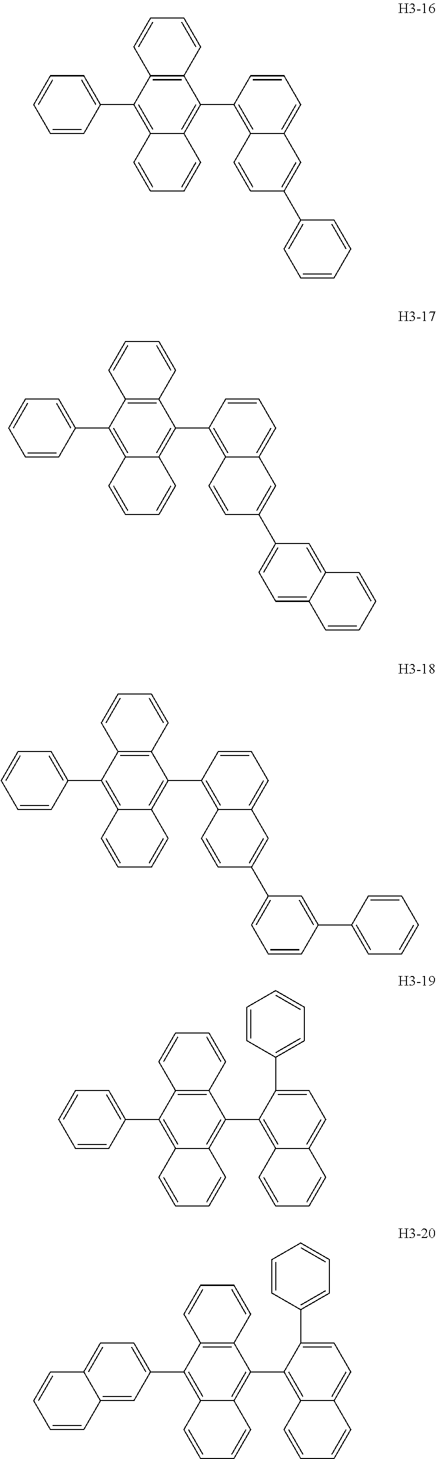

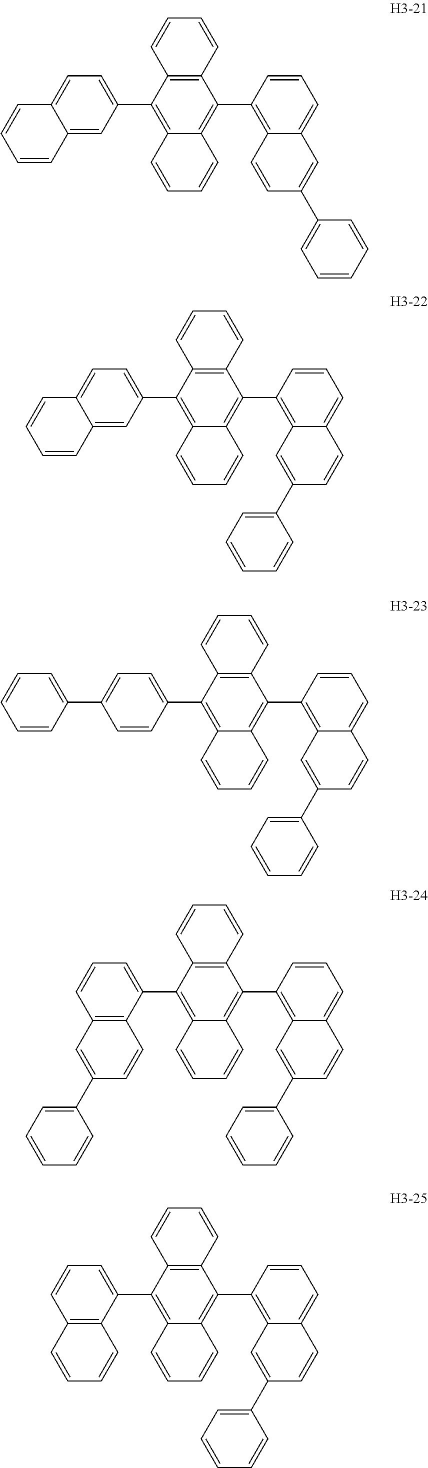

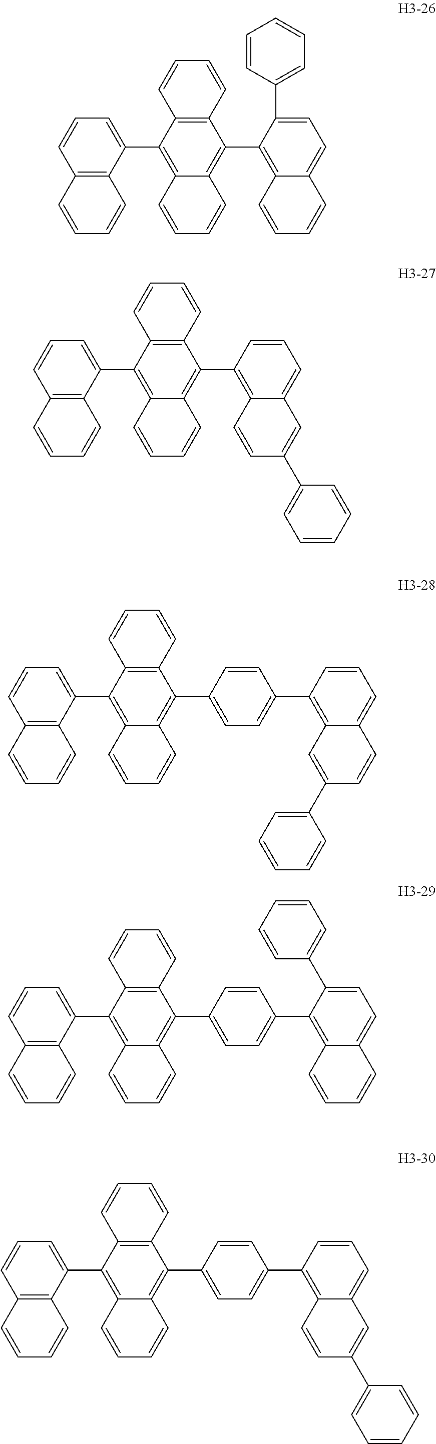

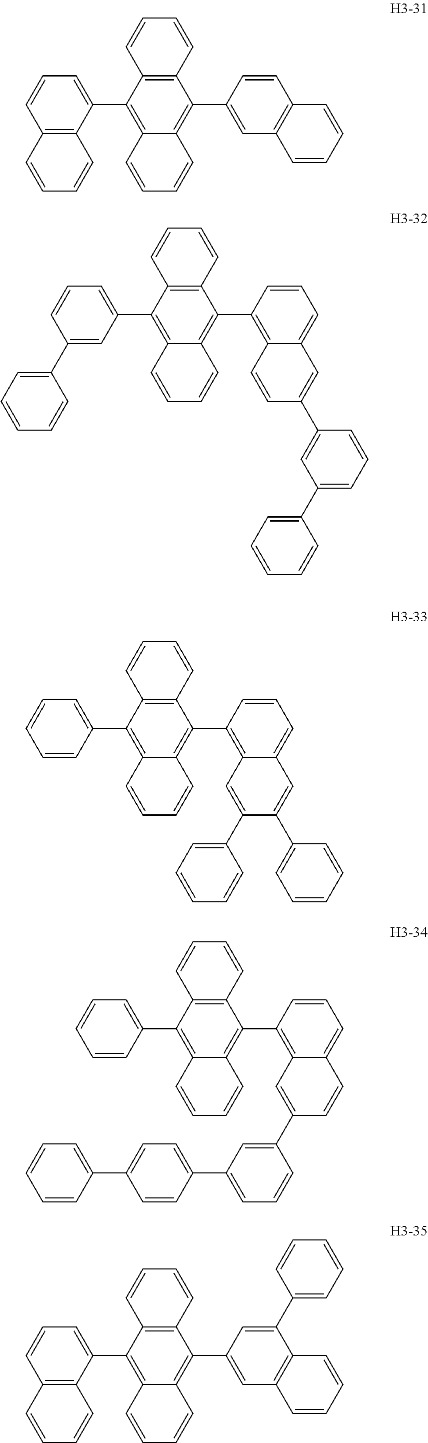

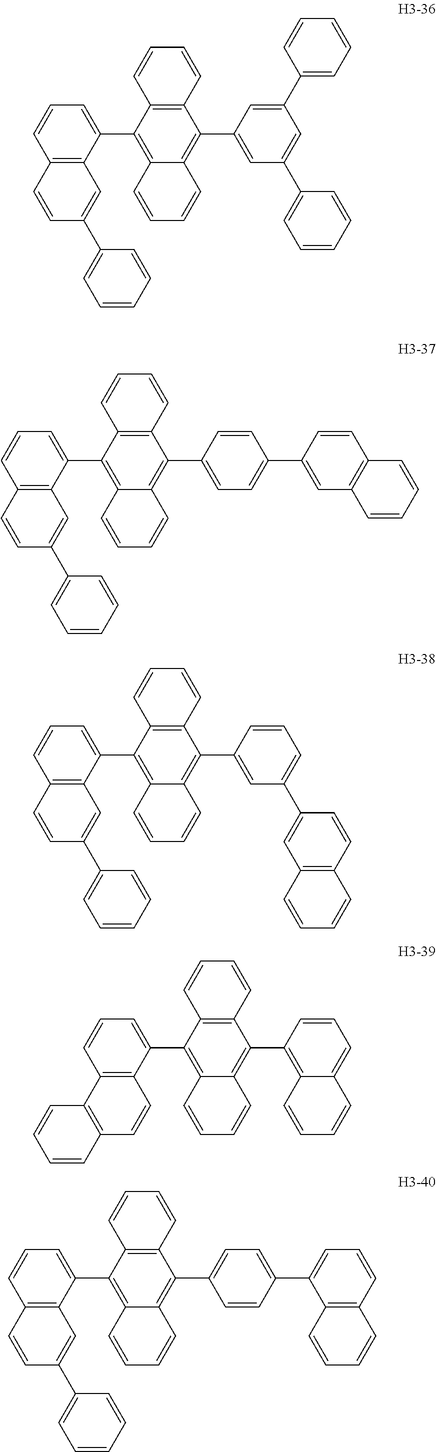

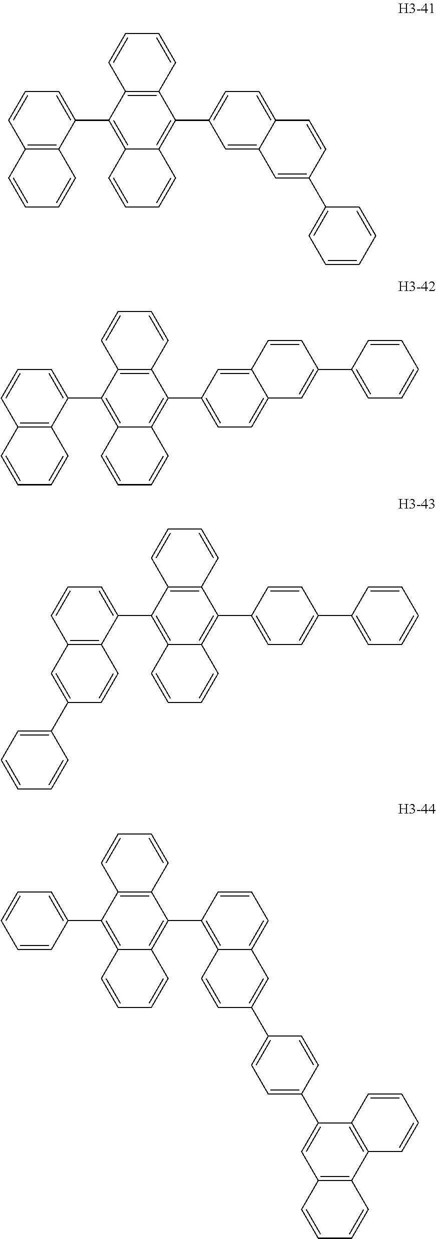

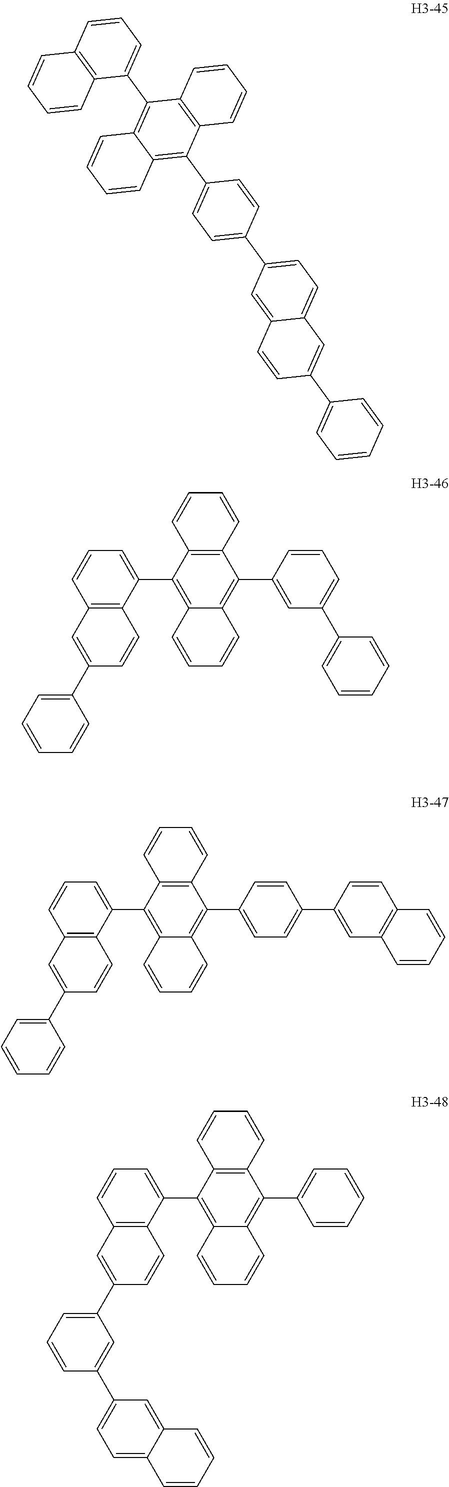

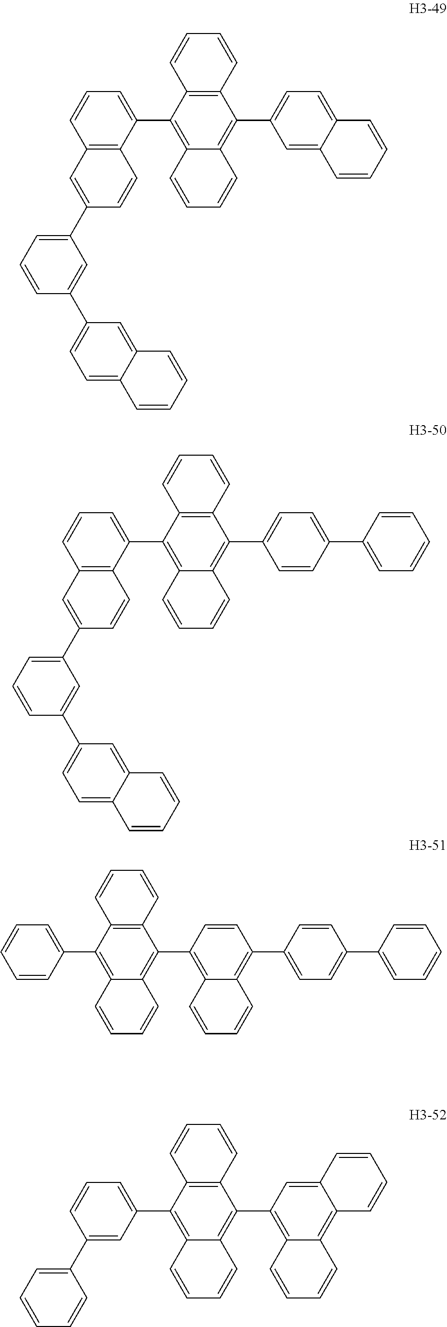

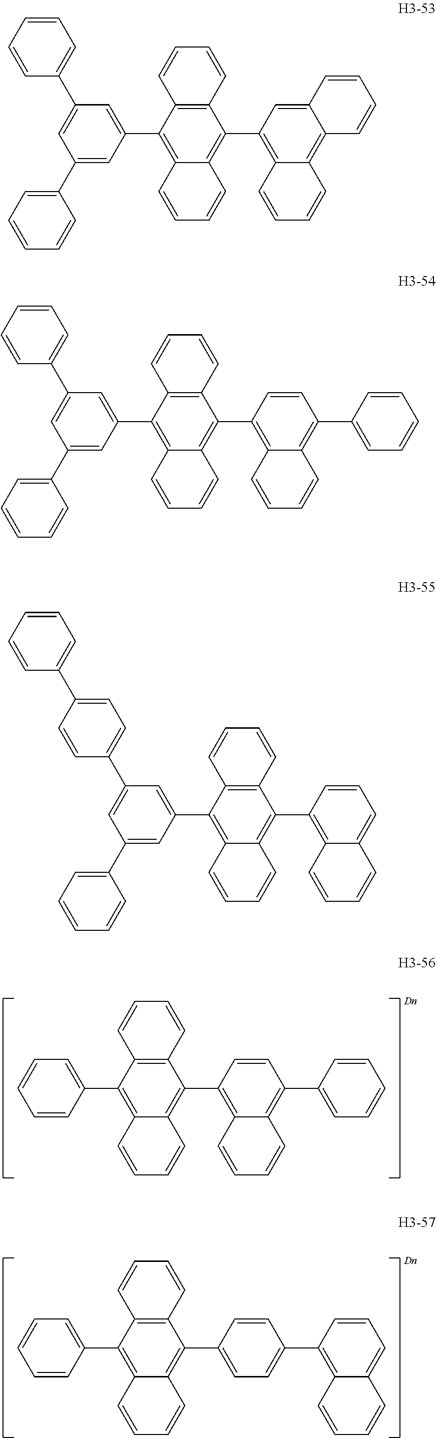

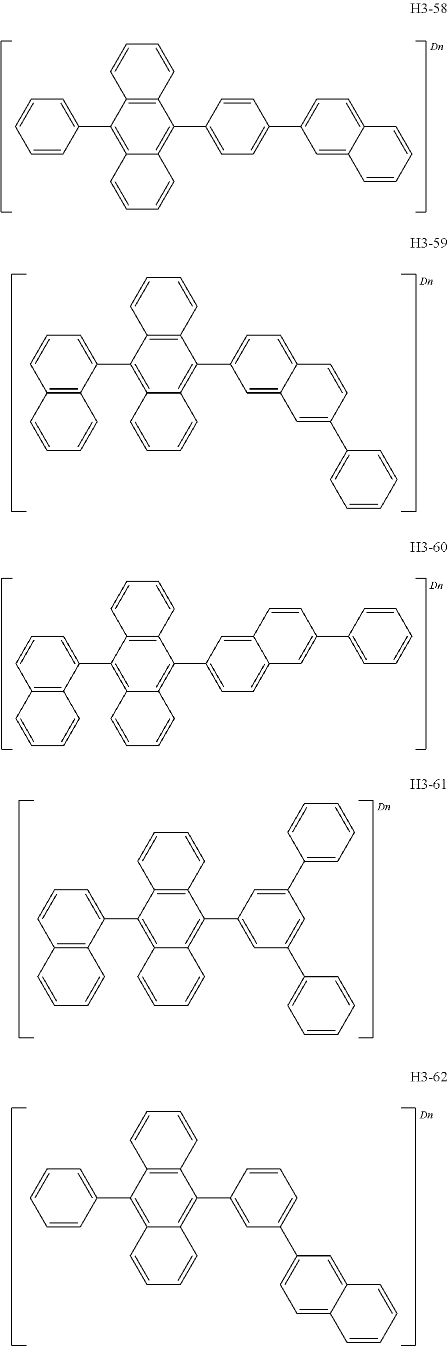

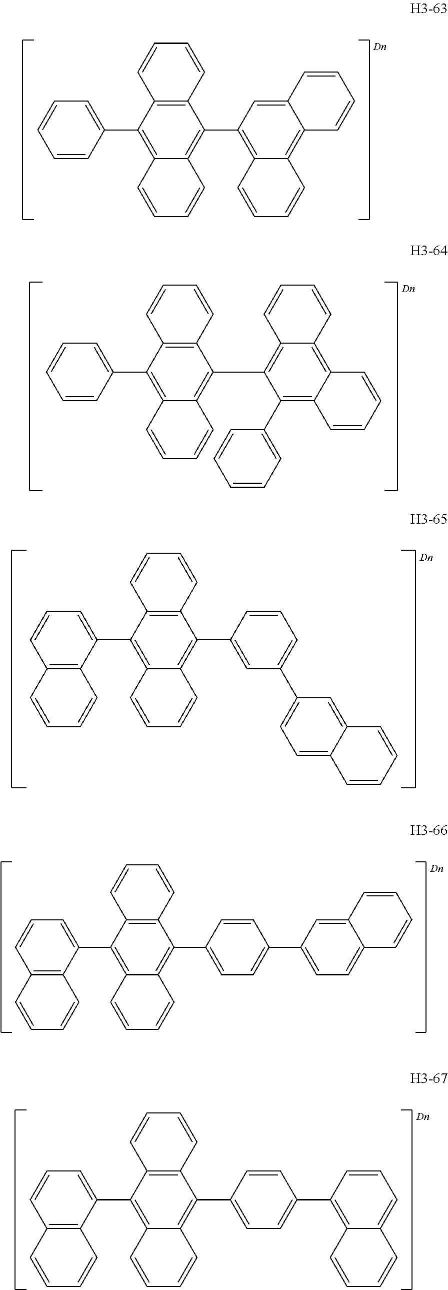

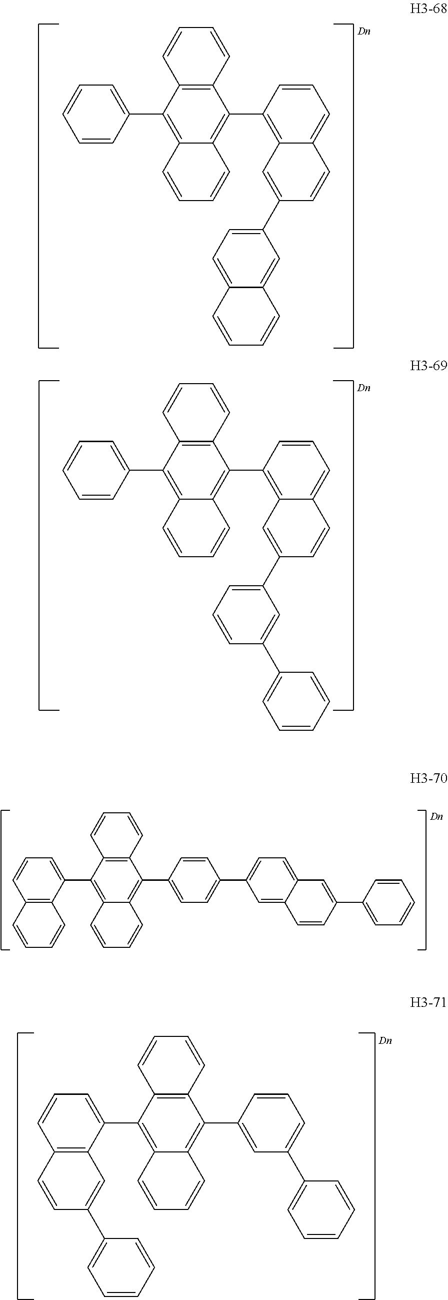

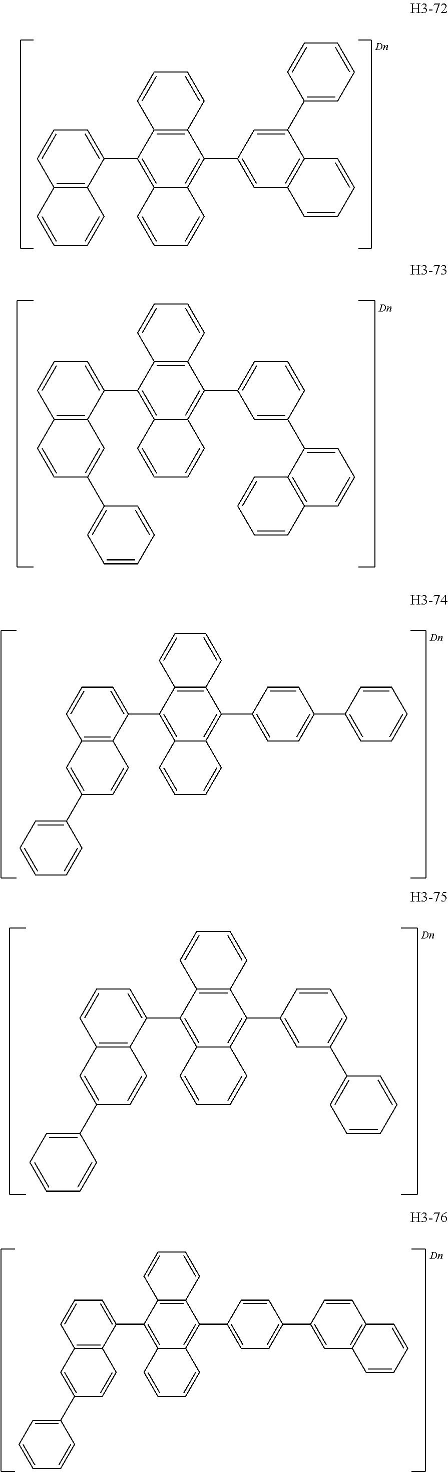

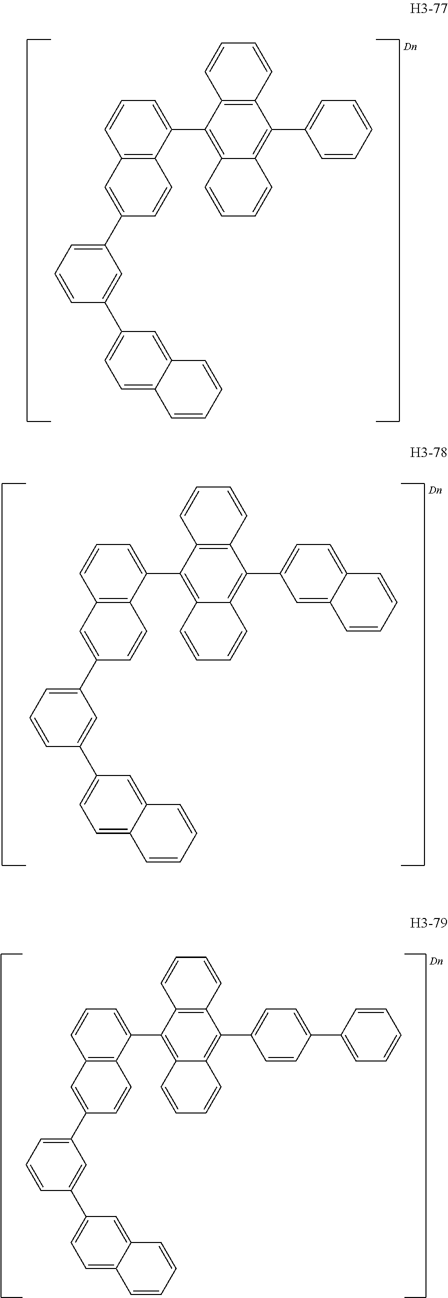

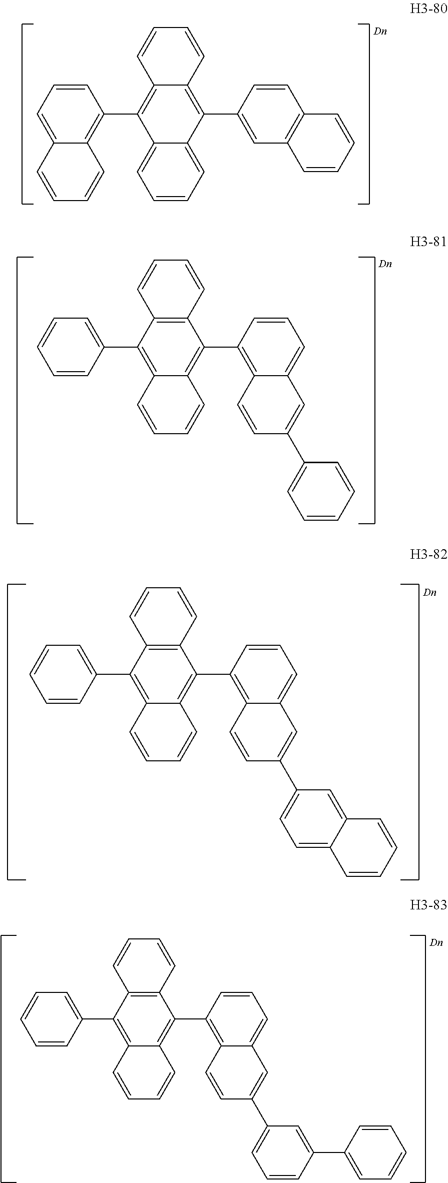

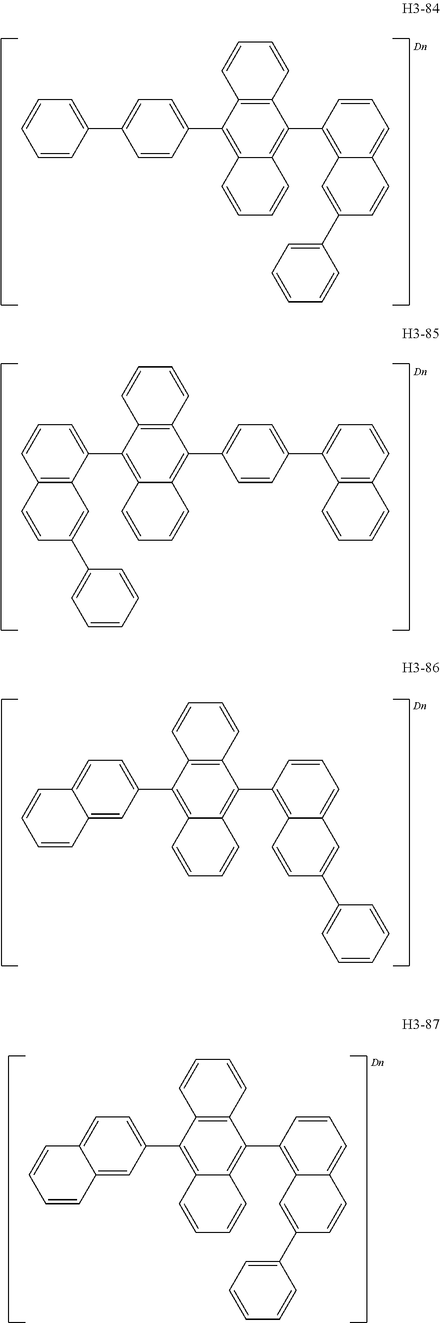

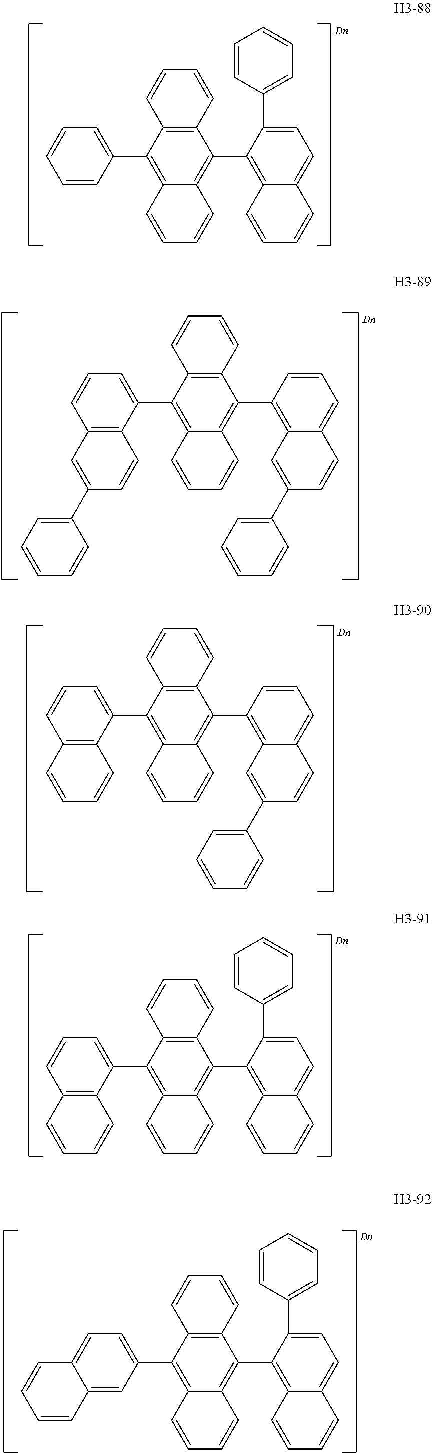

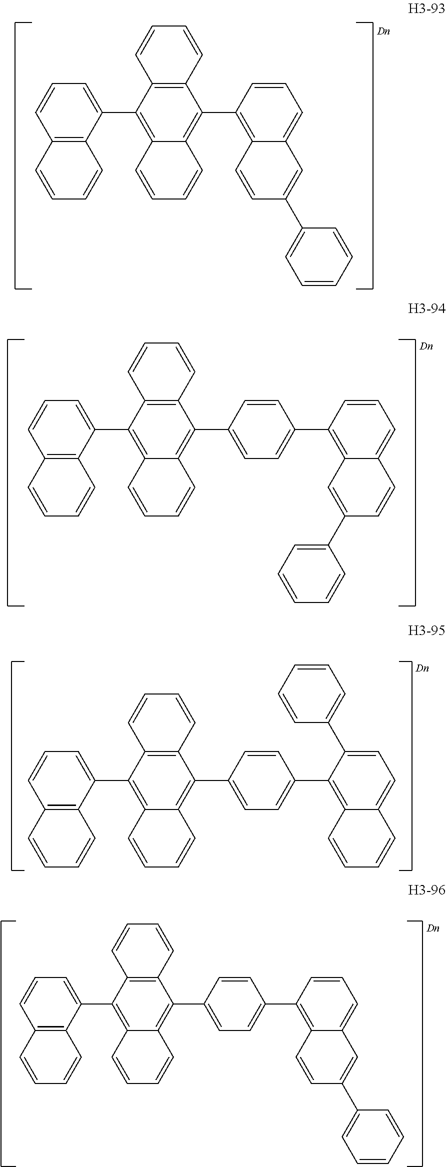

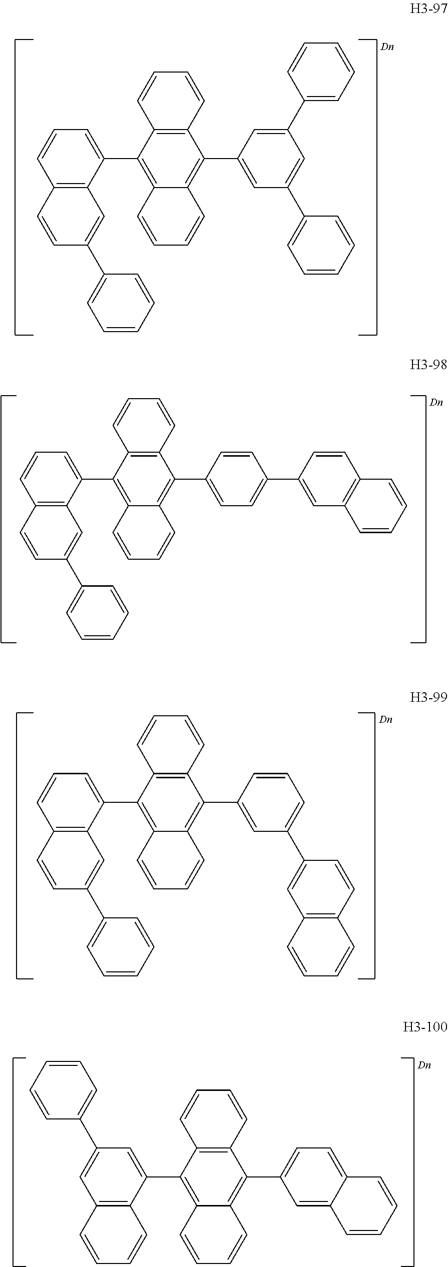

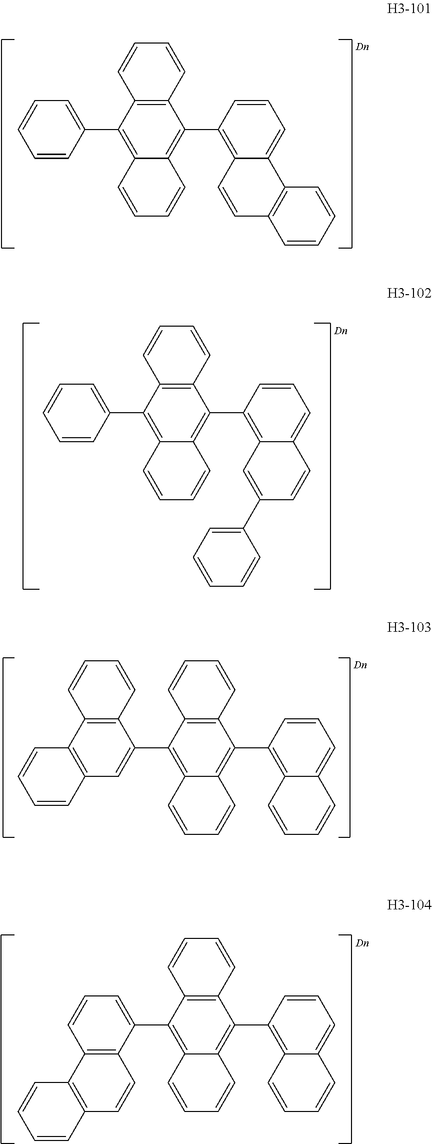

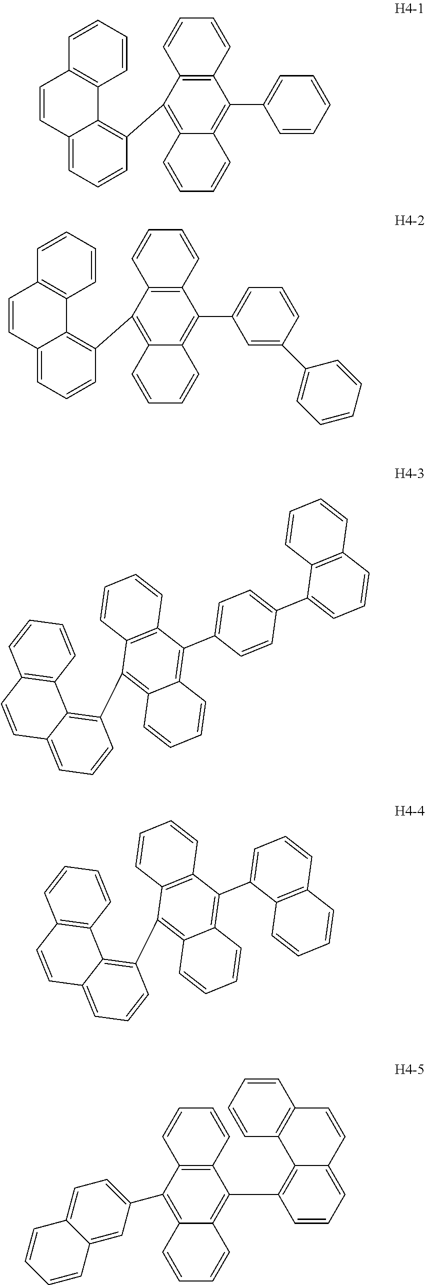

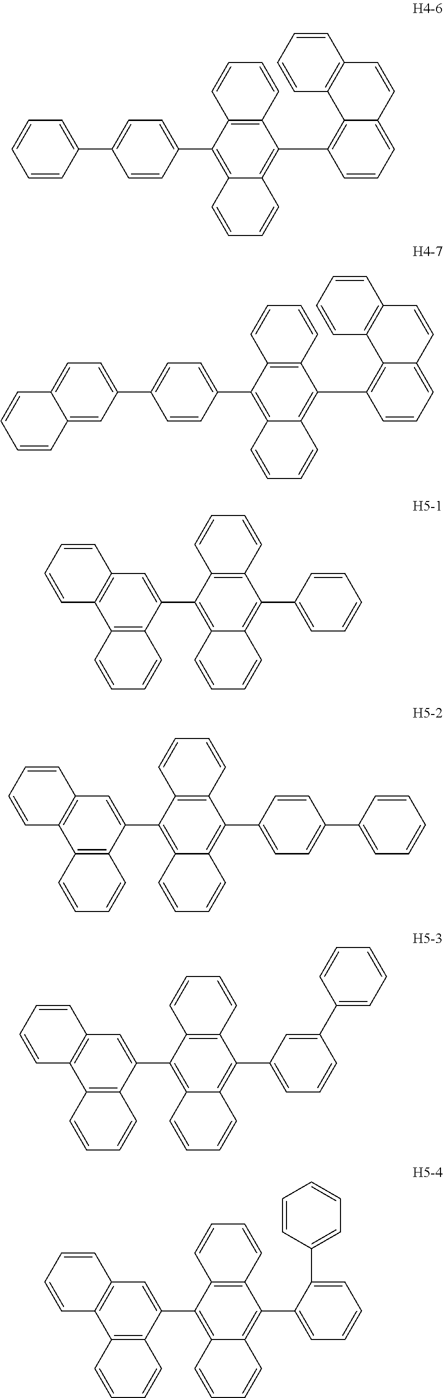

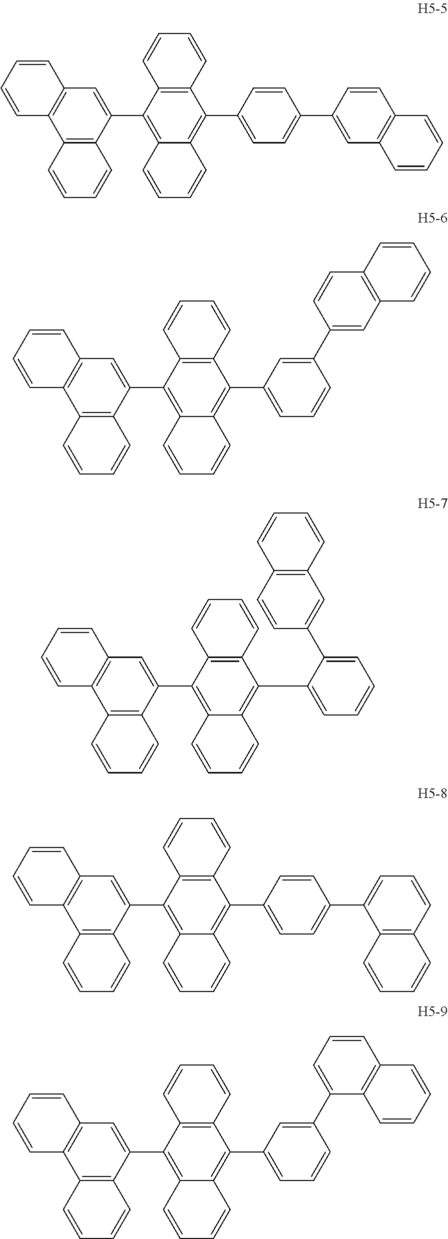

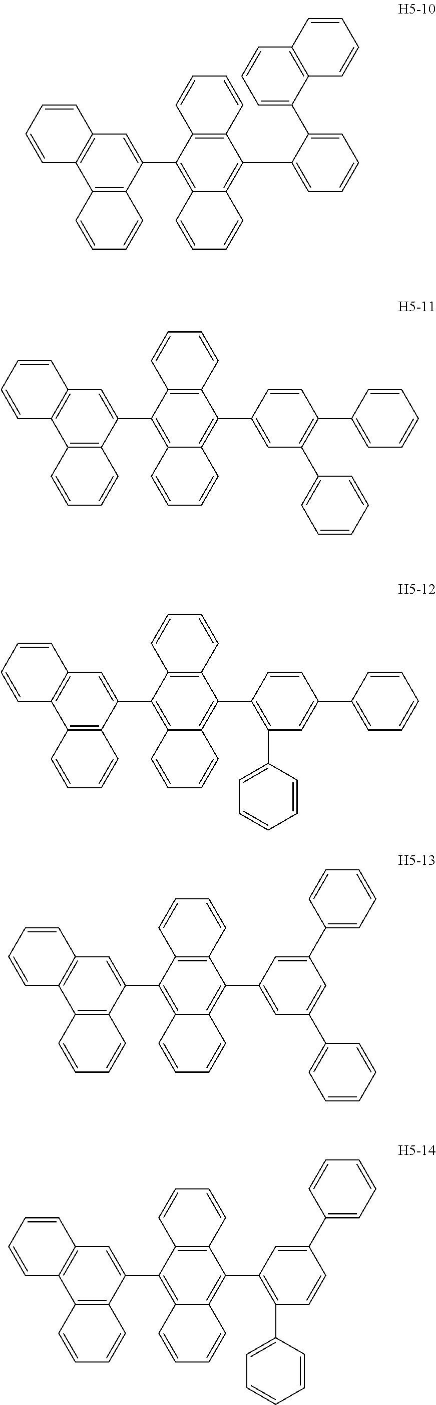

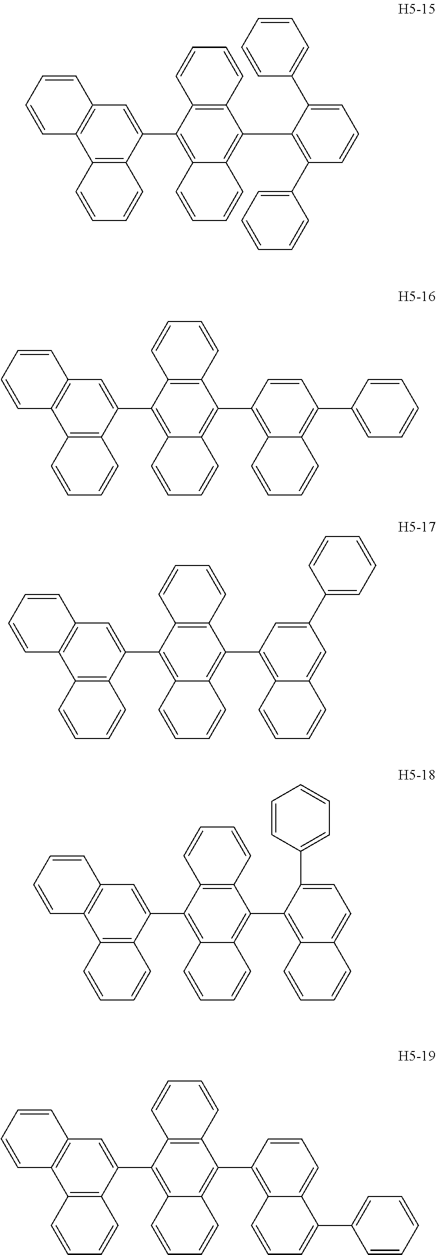

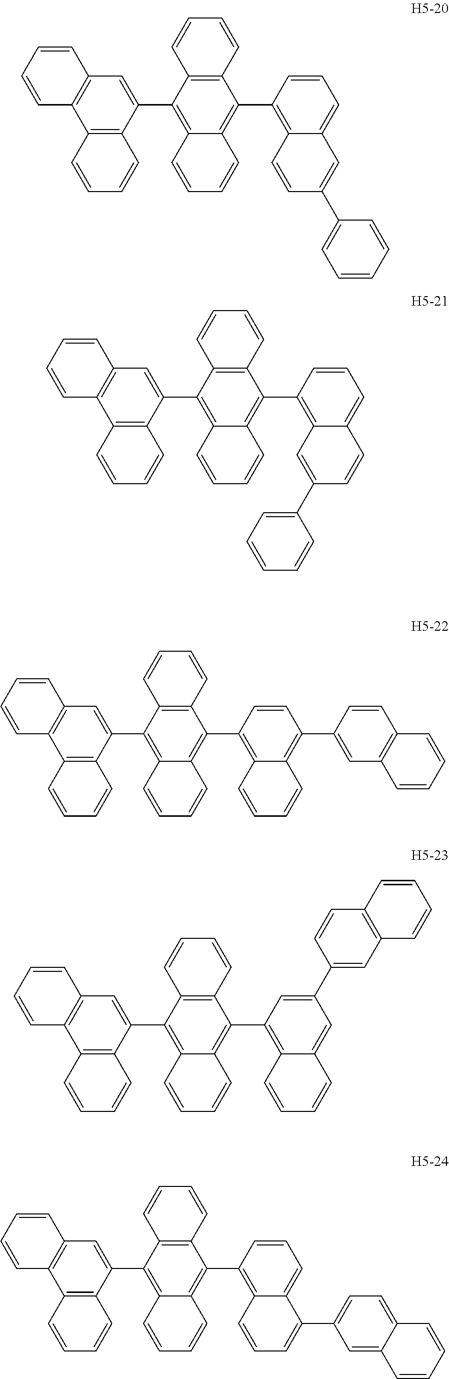

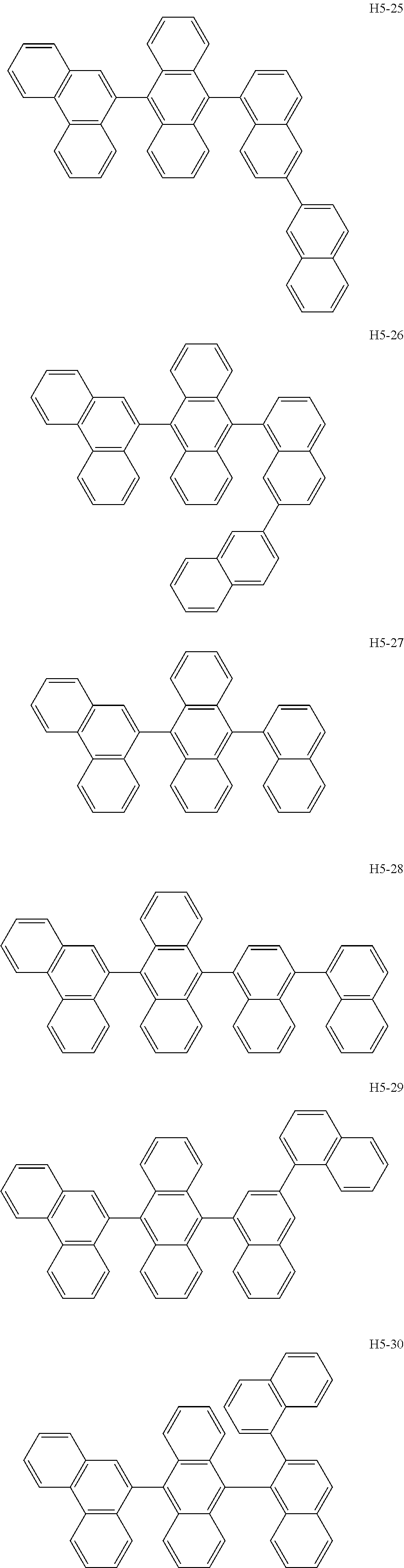

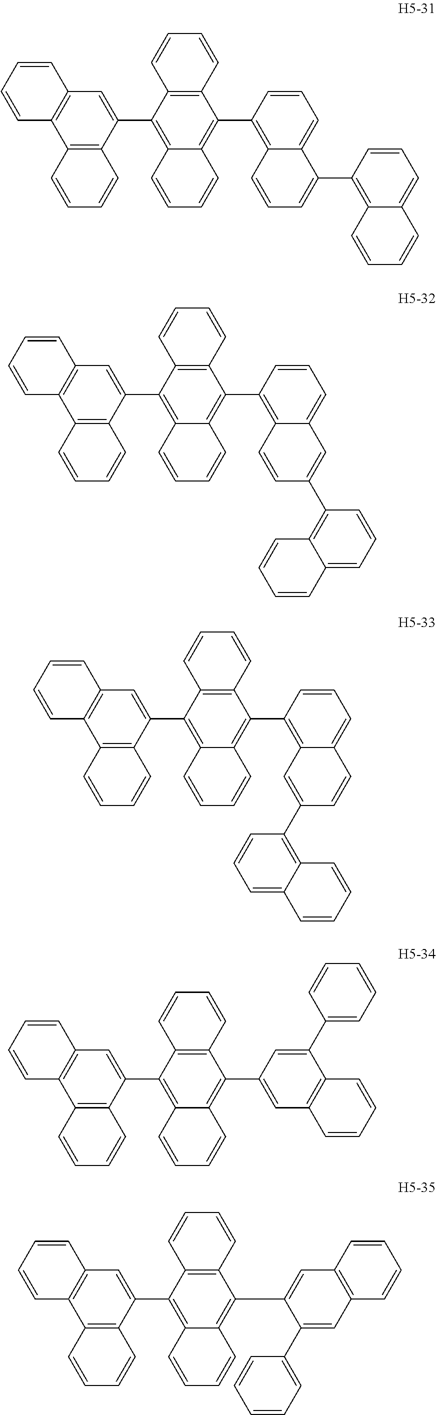

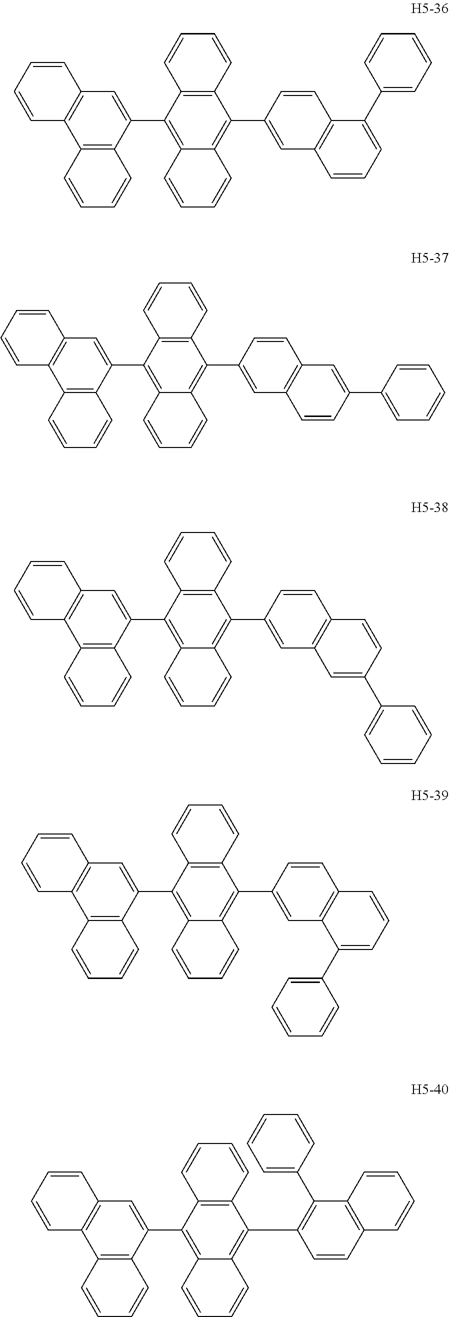

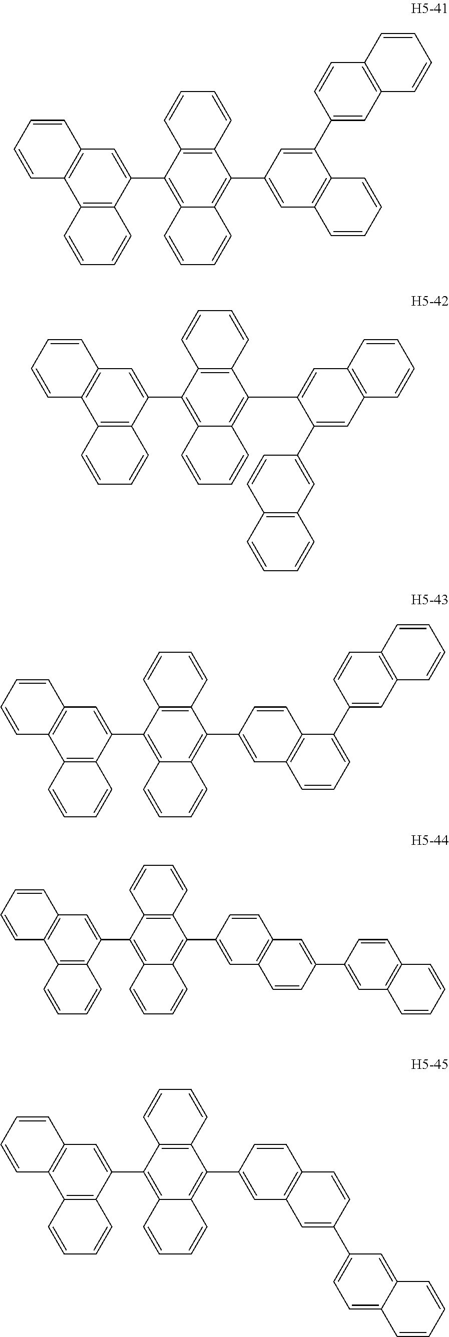

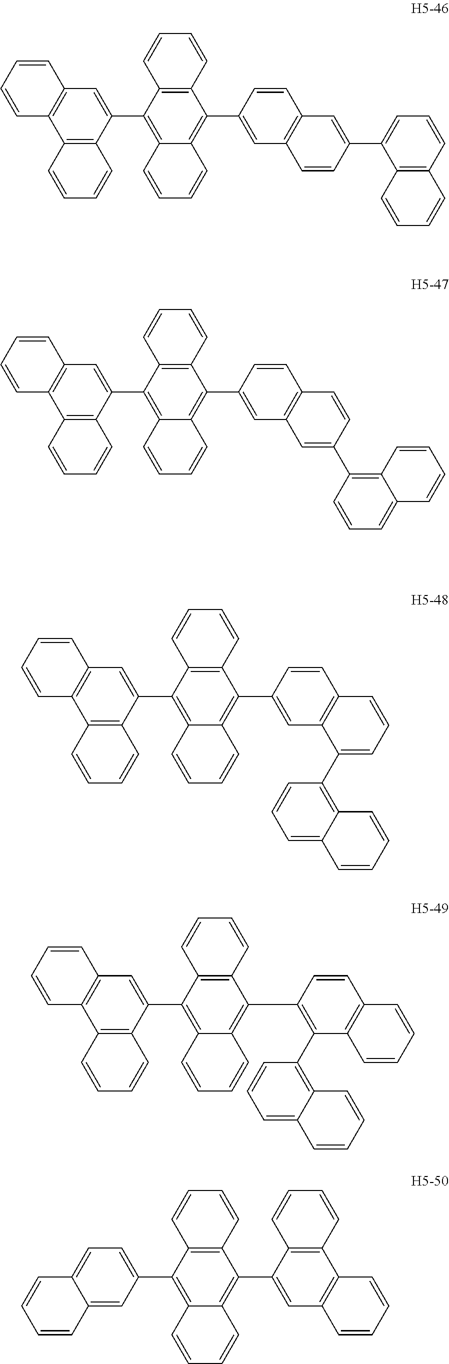

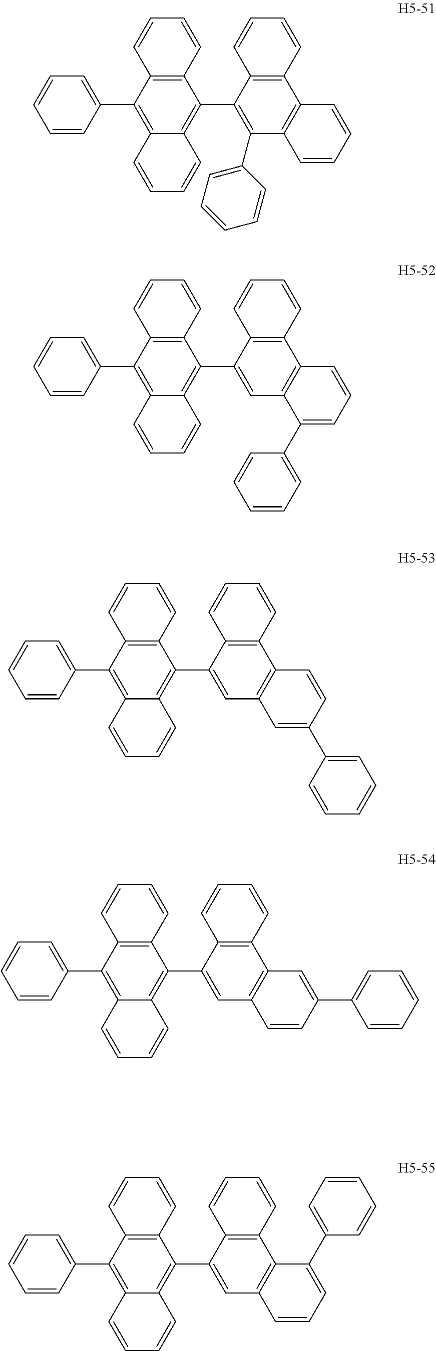

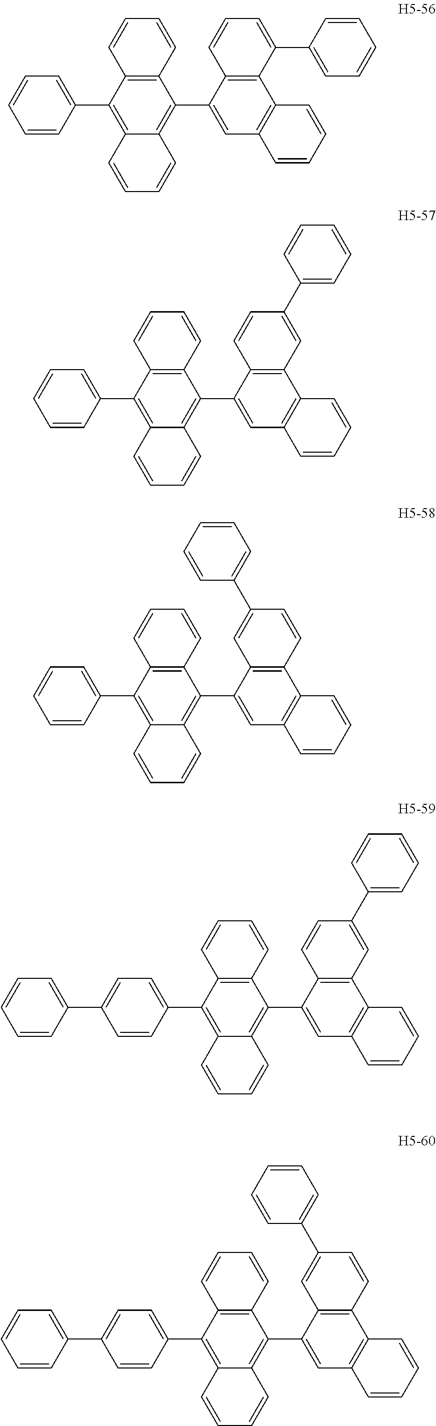

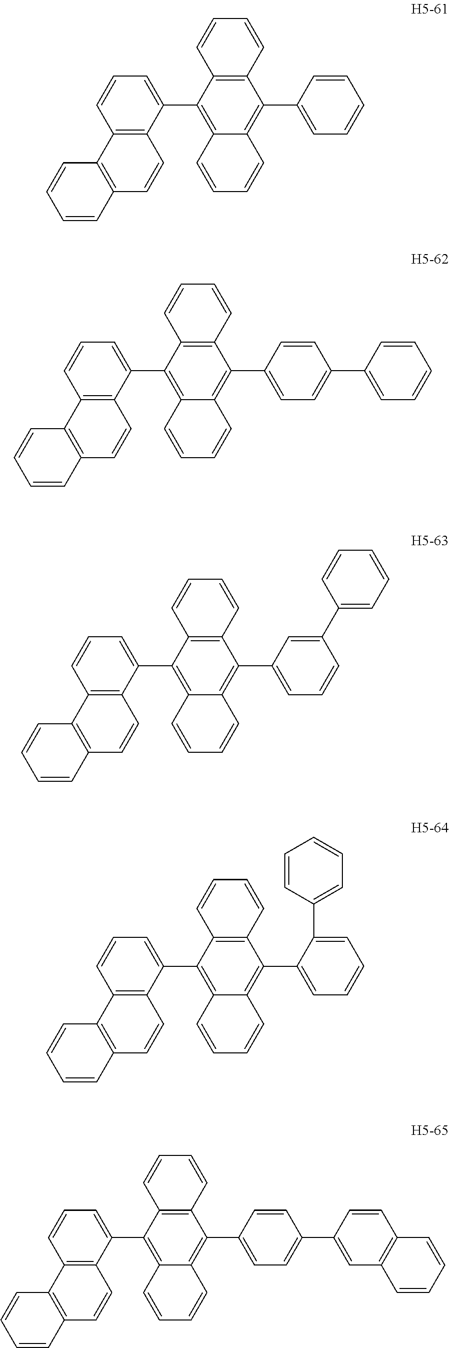

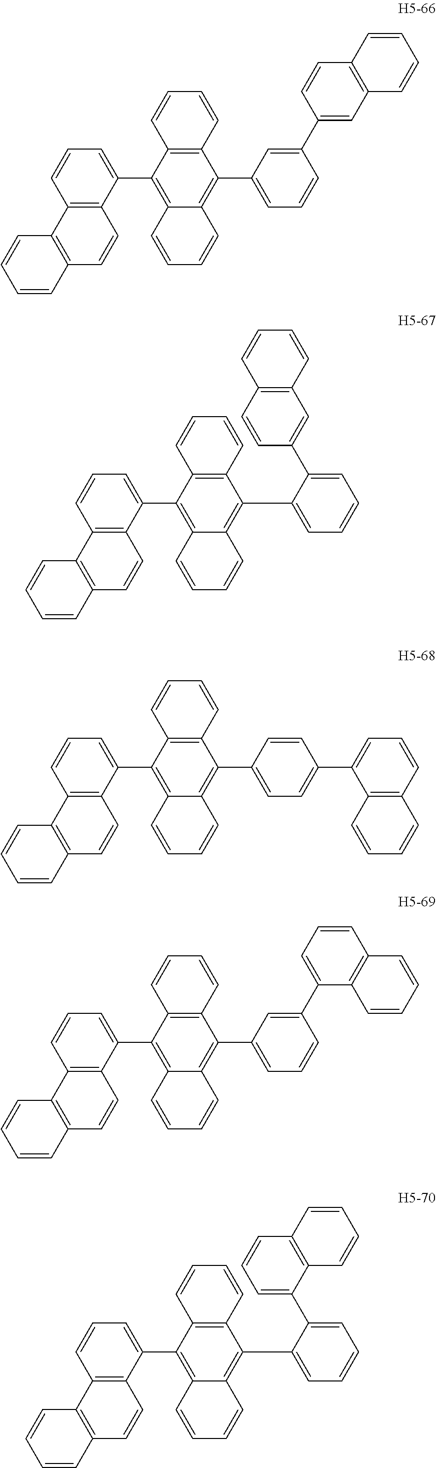

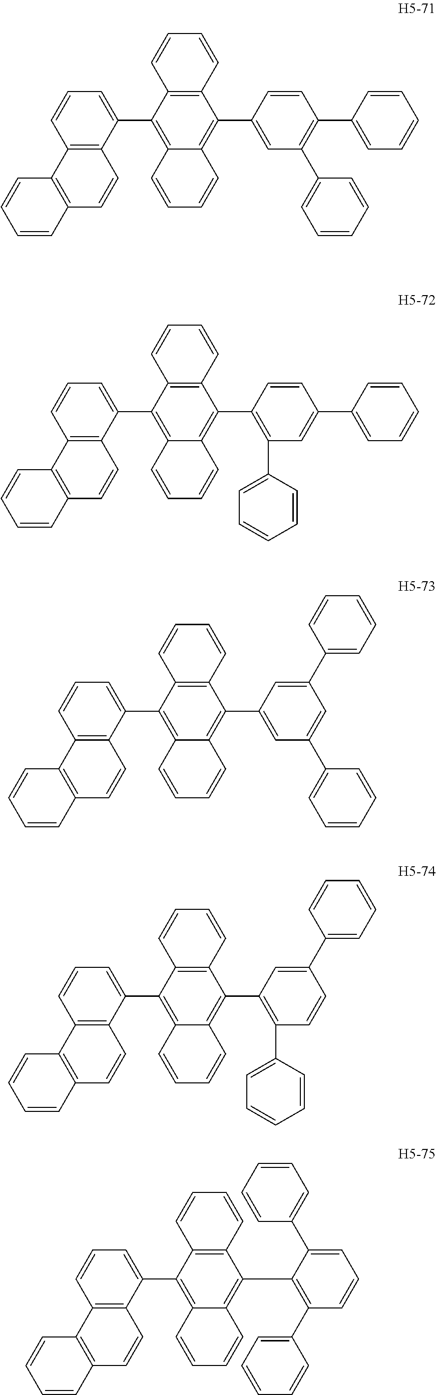

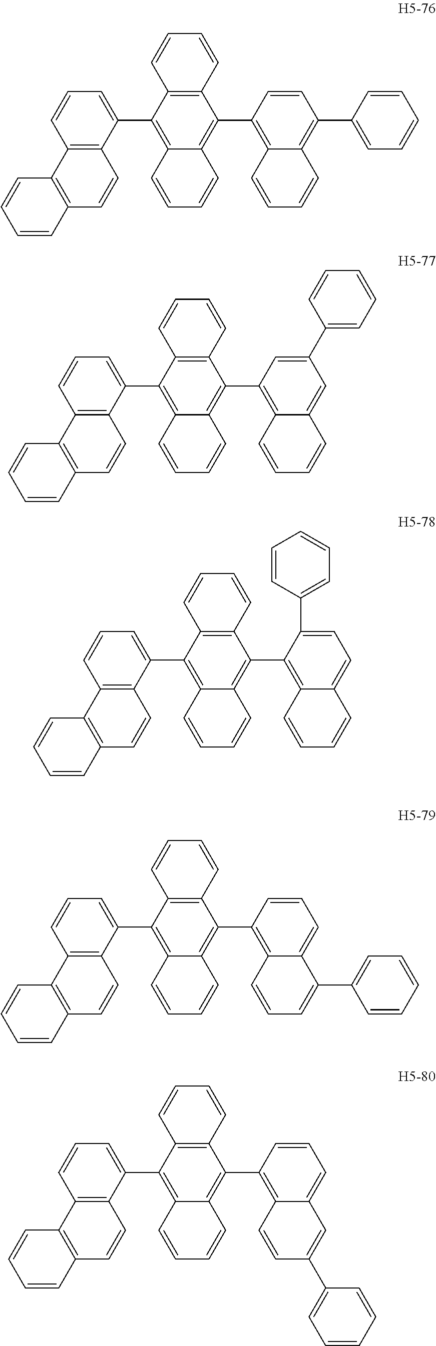

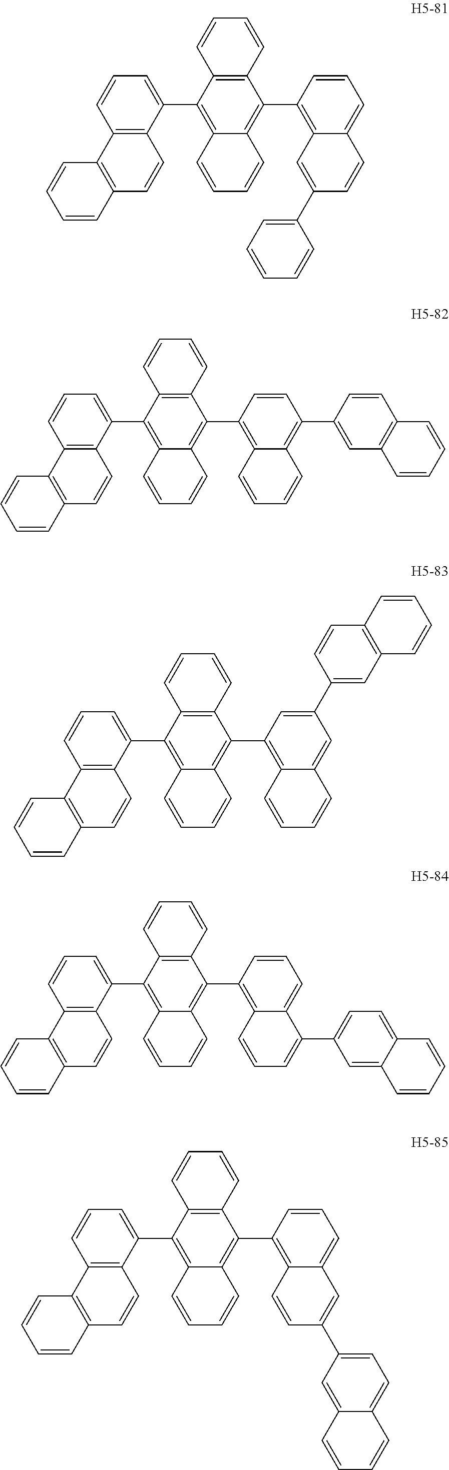

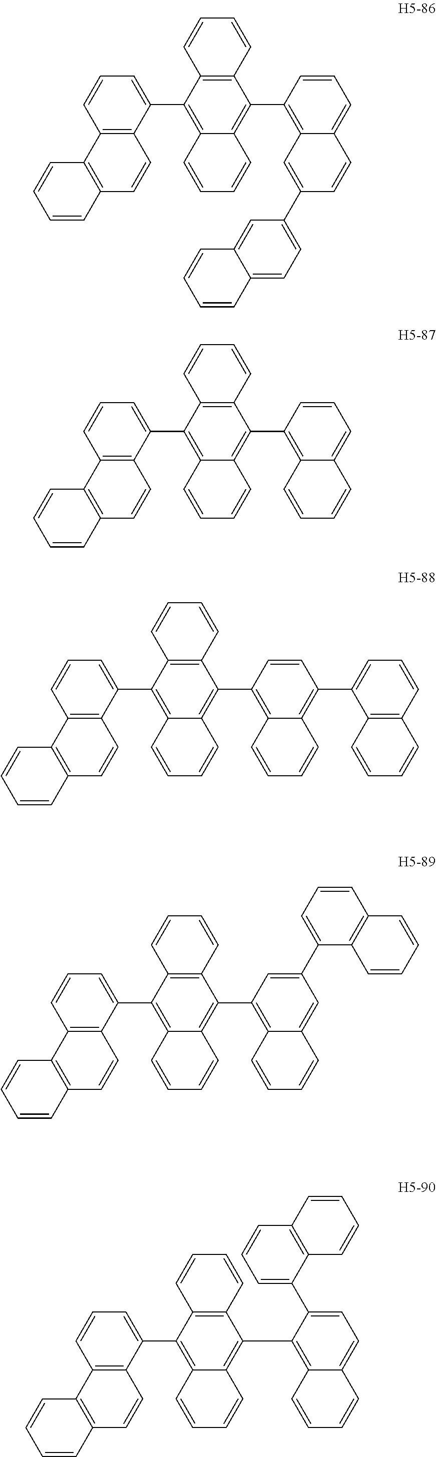

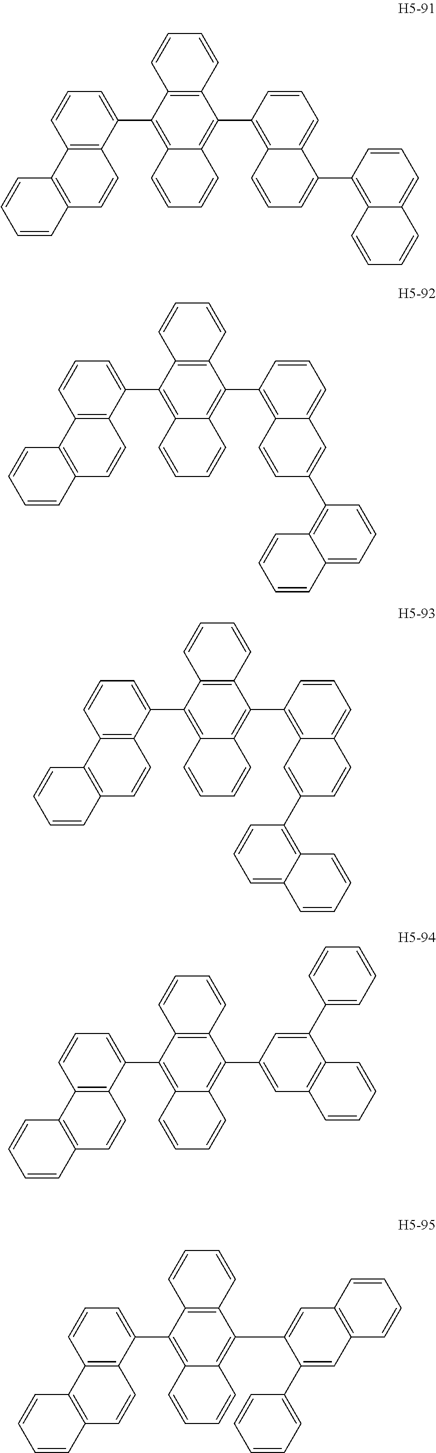

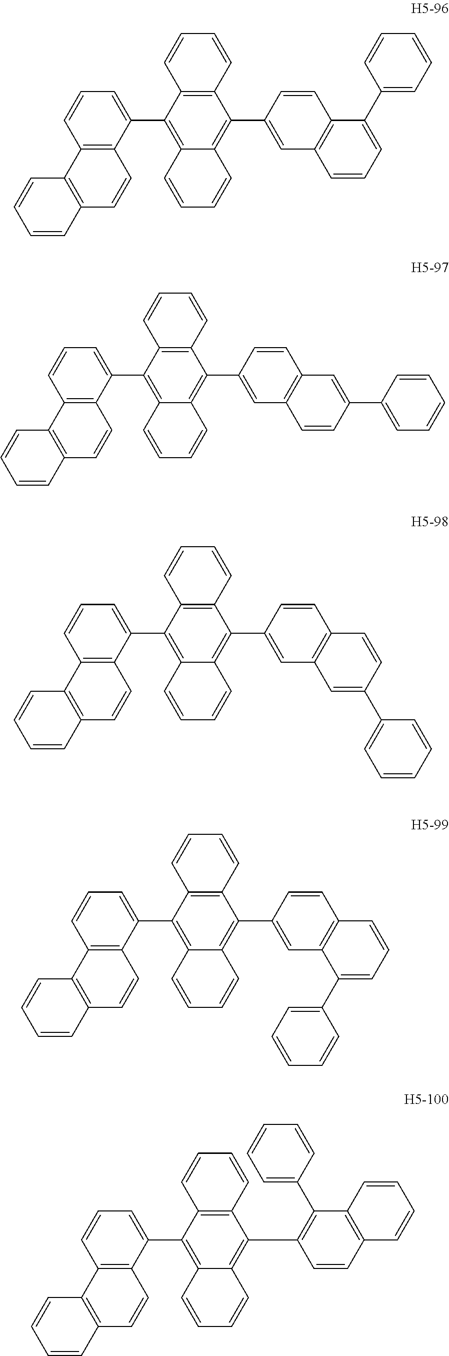

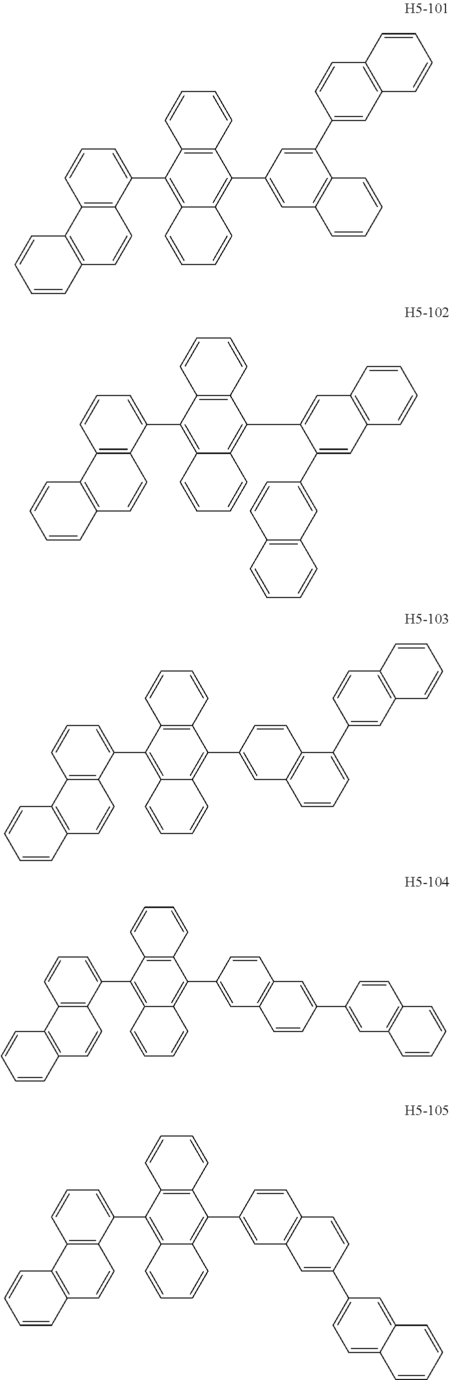

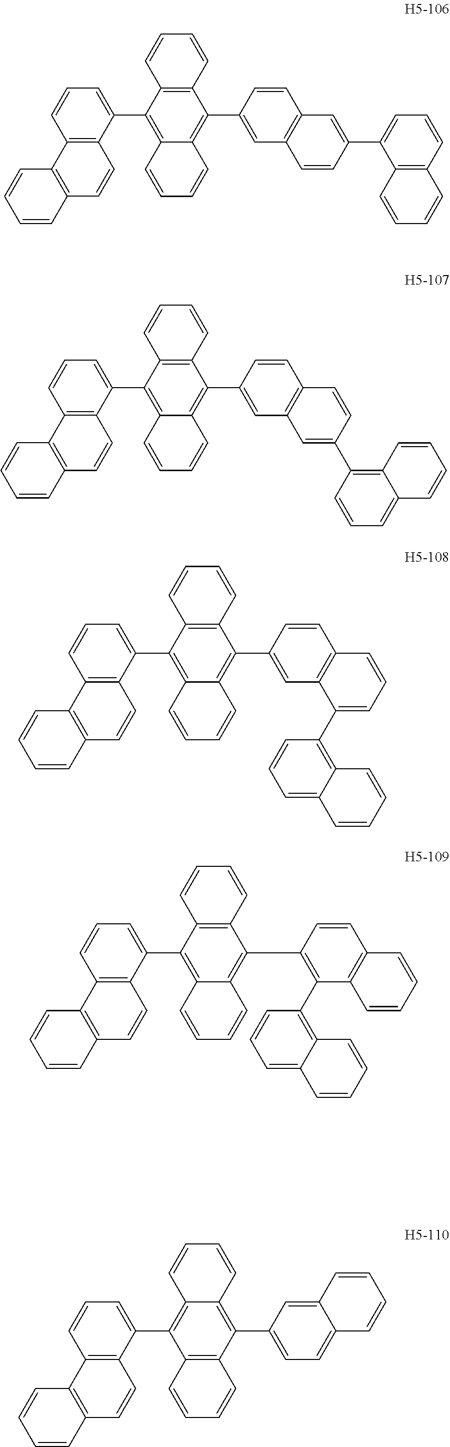

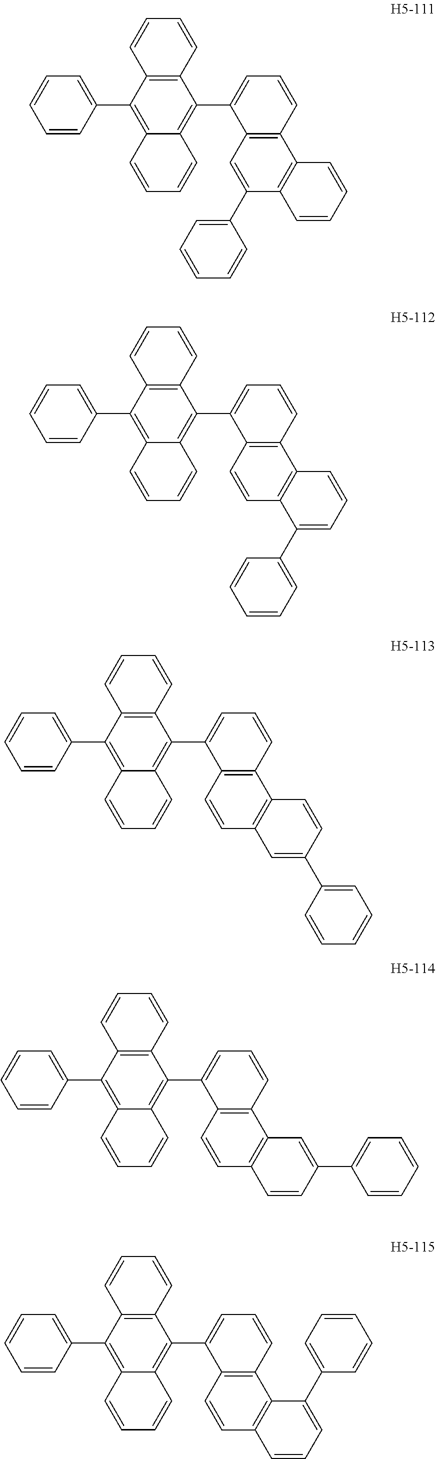

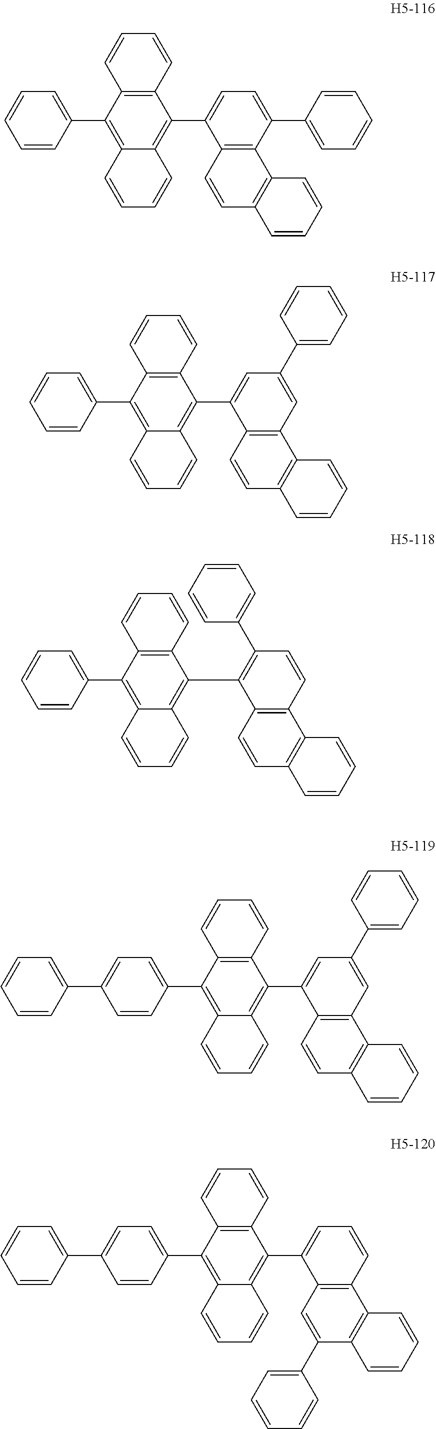

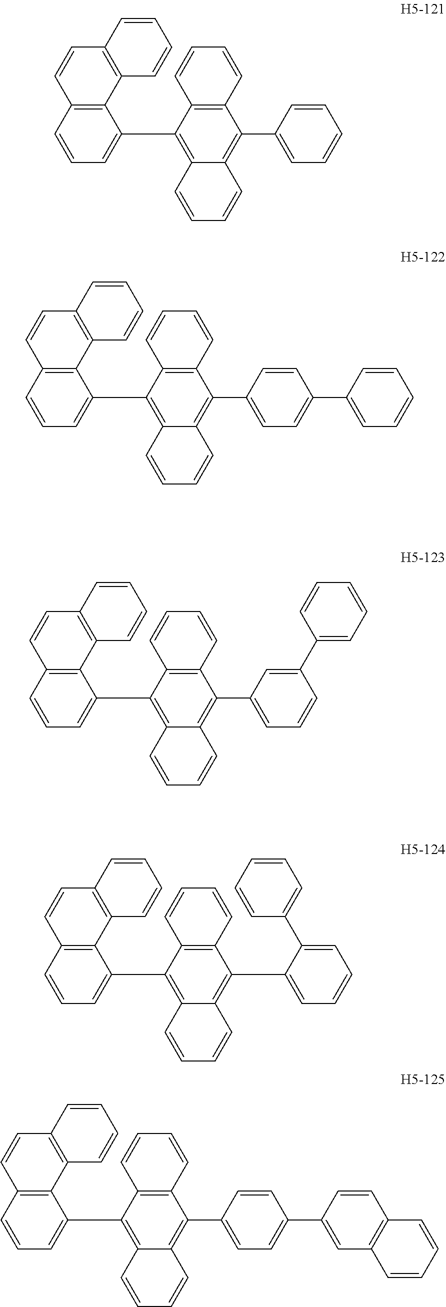

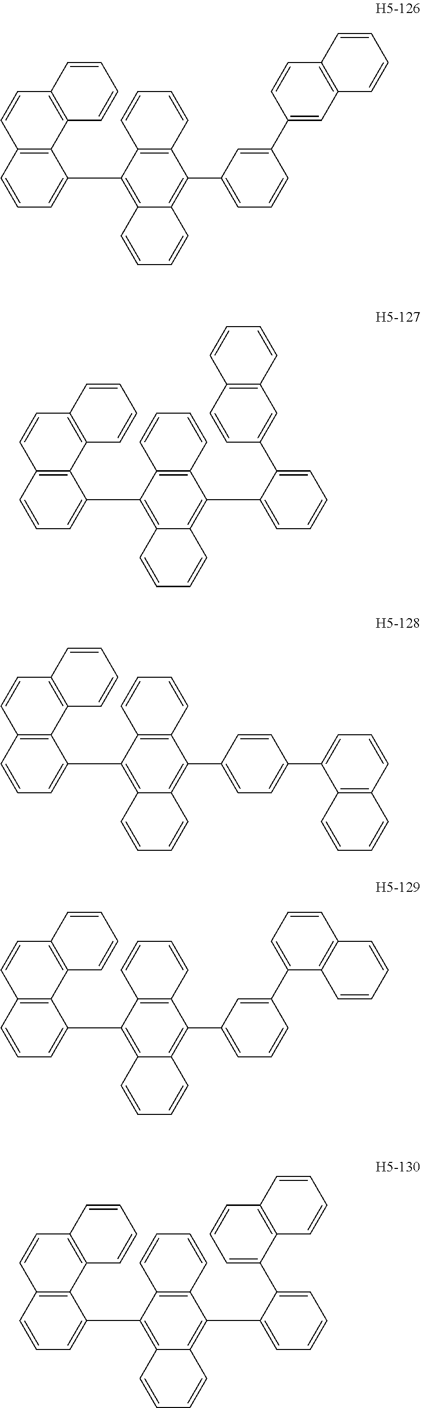

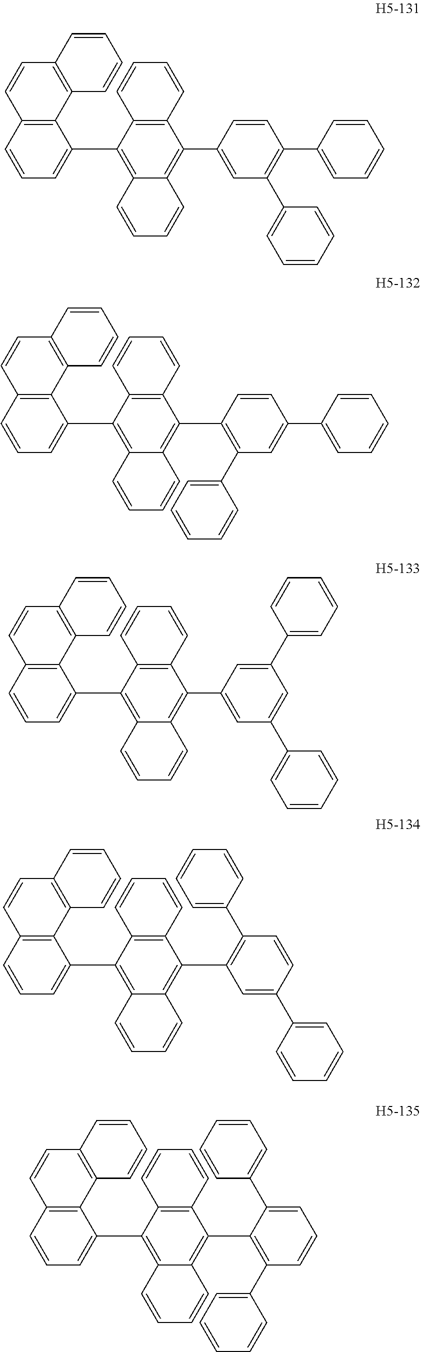

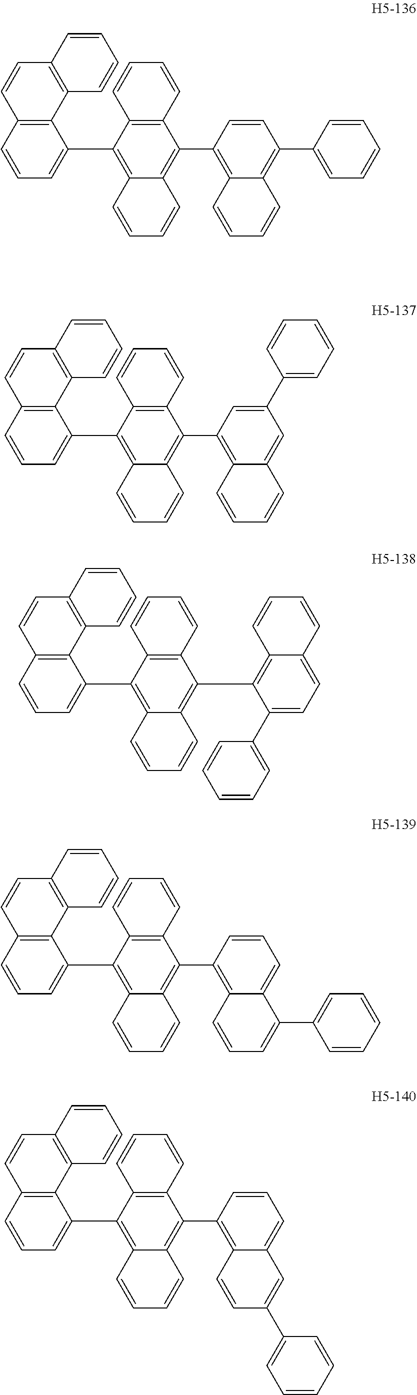

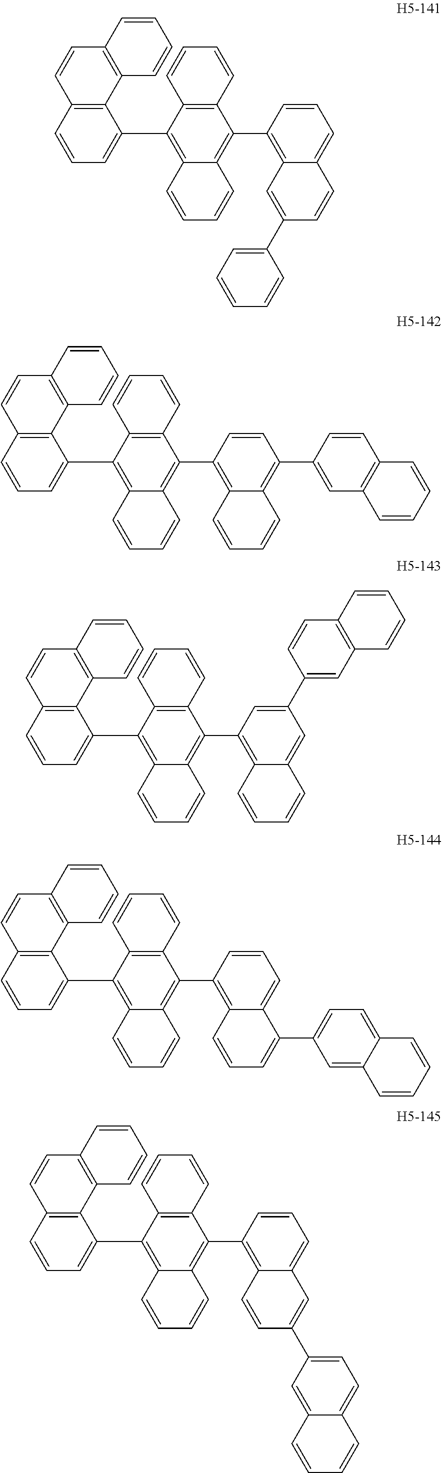

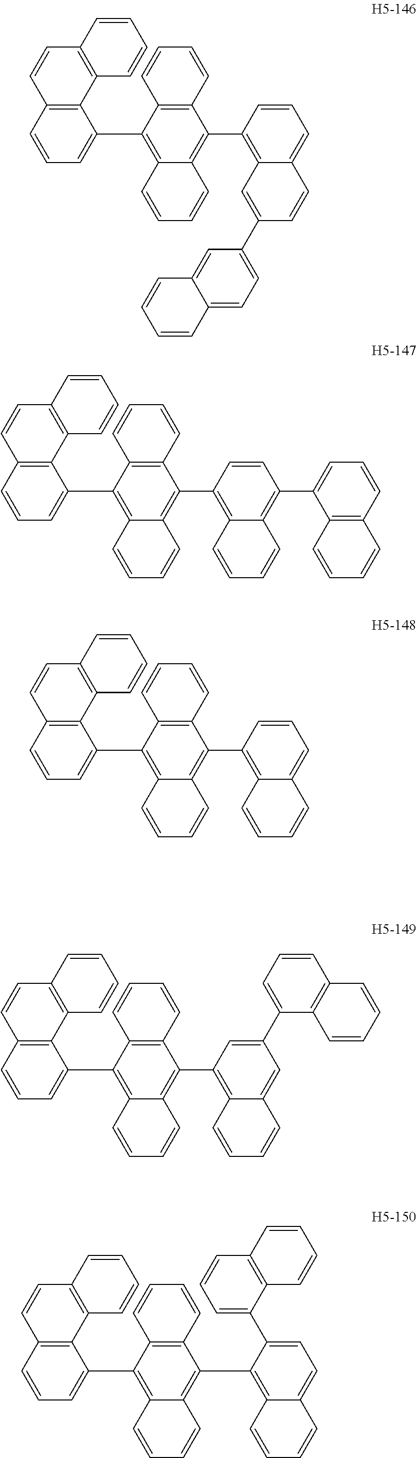

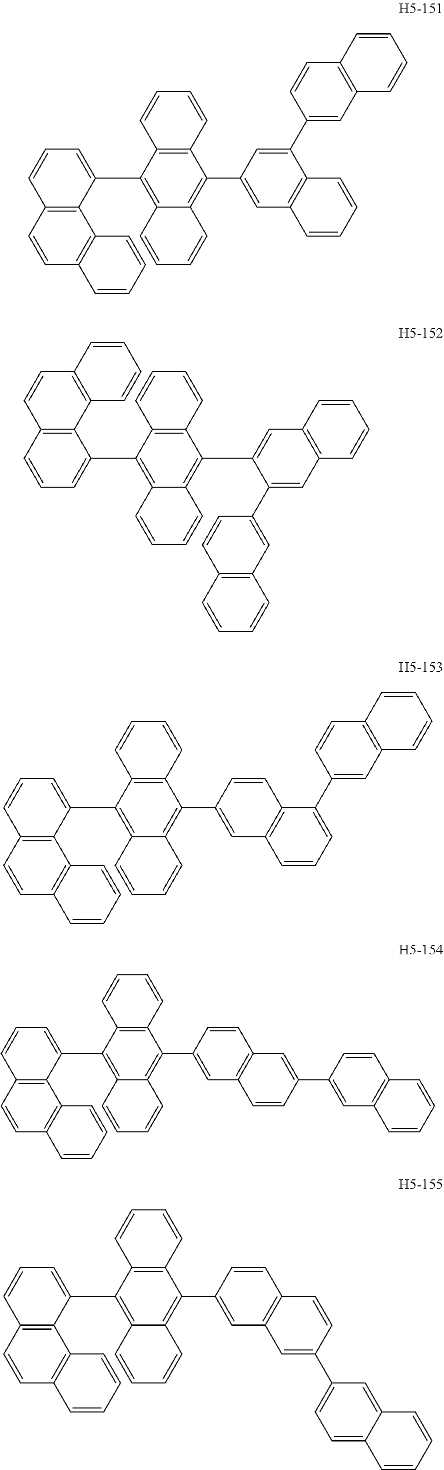

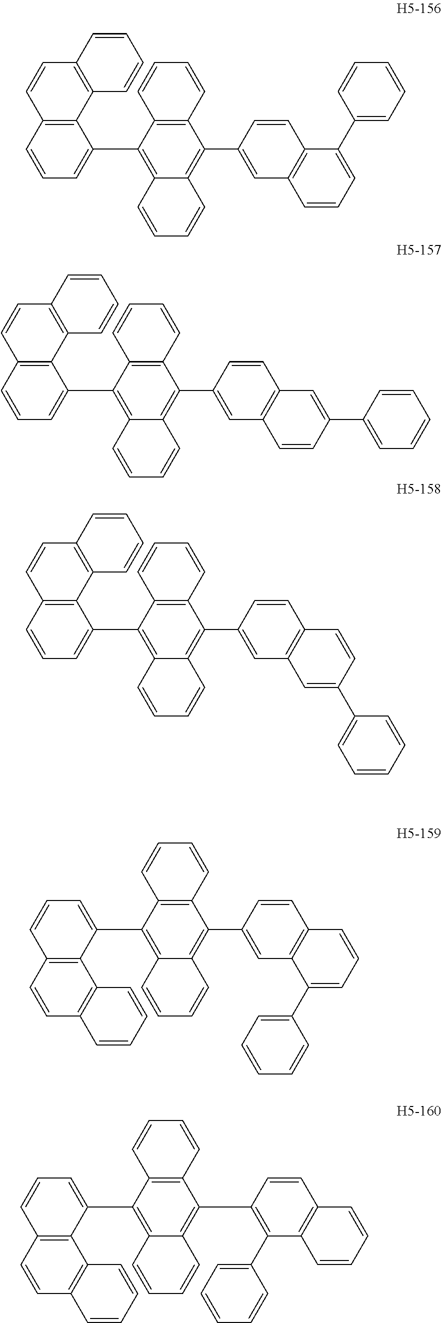

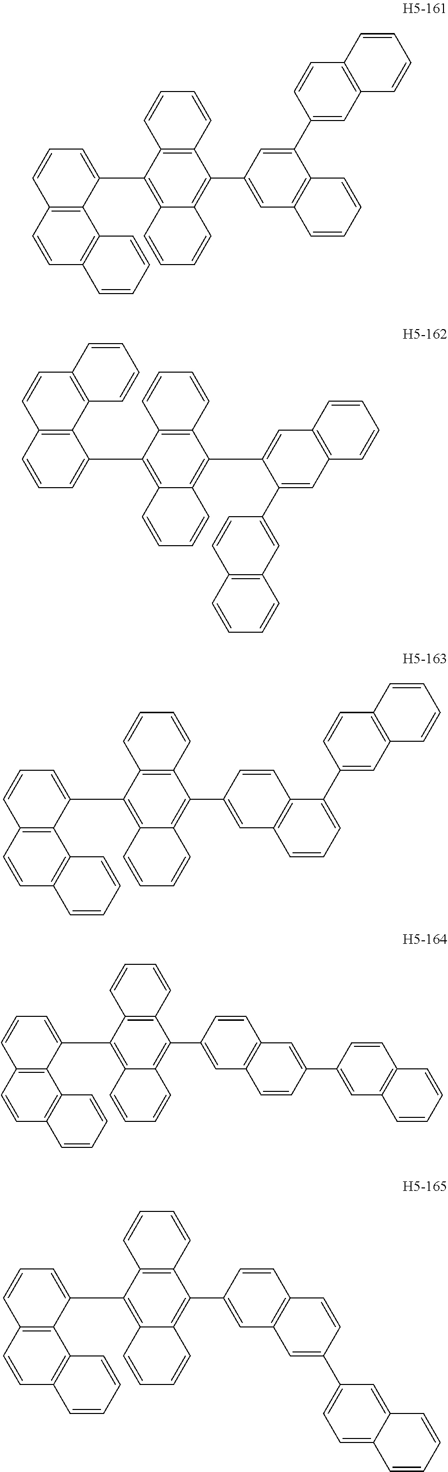

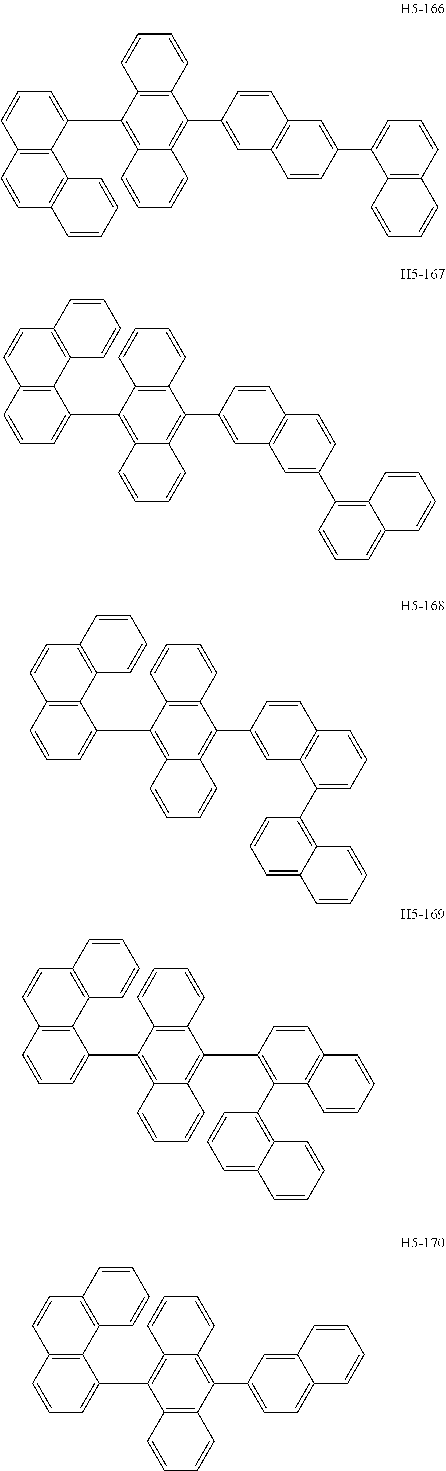

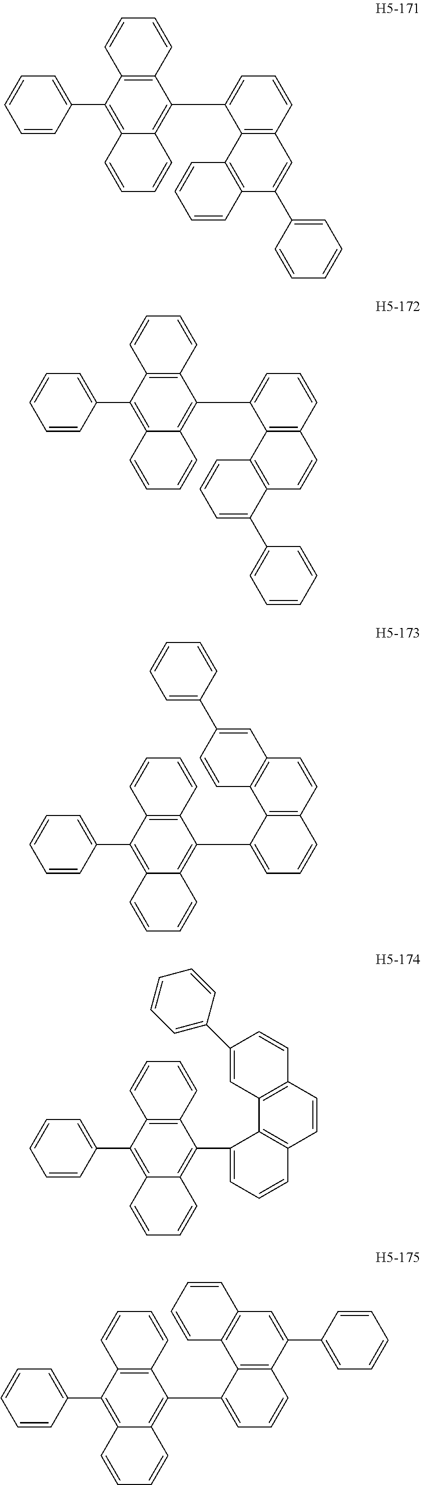

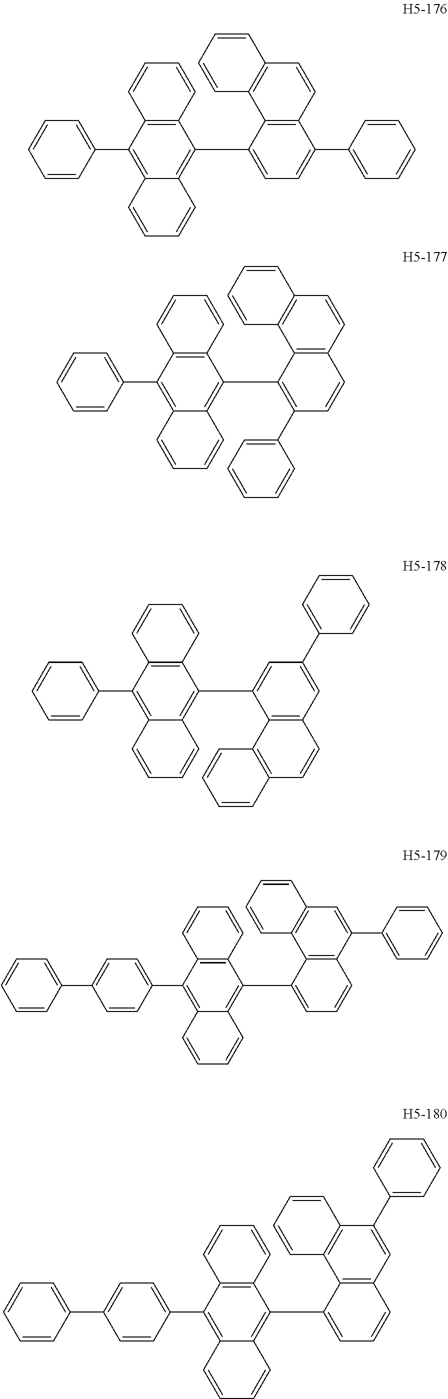

In the compounds, Dₙ indicates that n hydrogen atoms are replaced with deuterium, and n ranges from 1 to the total number of hydrogen atoms in the compound.

Hereinafter, an organic electroluminescent device to which the aforementioned organic electroluminescent compound is applied will be described.

According to another embodiment of the present disclosure, an organic electroluminescent device is provided, comprising: a first electrode; a second electrode opposing the first electrode; and a plurality of light-emitting layers disposed between the first electrode and the second electrode. The light-emitting layers include a first light-emitting layer and a second light-emitting layer, which are adjacent to each other, wherein the first light-emitting layer includes an organic electroluminescent compound represented by Formula 1 as a first host compound. The first light-emitting layer may further include an additional host compound in addition to the organic electroluminescent compound represented by Formula 1.

According to one embodiment, the second light-emitting layer includes an organic electroluminescent compound represented by Formula 2 as a second host compound. The second light-emitting layer may also further include an additional host compound in addition to the organic electroluminescent compound represented by Formula 2.

According to another embodiment, both the first light-emitting layer and the second light-emitting layer may further include an additional host compound.

In one embodiment, both the first light-emitting layer and the second light-emitting layer may be blue light-emitting layers.

An organic electroluminescent device according to one embodiment may further include a third light-emitting layer adjacent to the second light-emitting layer.

An organic electroluminescent device according to one embodiment may further include an organic layer such as a hole transport layer, a light-emitting layer, a hole auxiliary layer, an electron-blocking layer, and a light-emitting auxiliary layer in addition to a first electrode; a second electrode; and a plurality of light-emitting layers disposed between the first electrode and the second electrode. The organic electroluminescent device may further include at least one layer selected from a hole injection layer, an electron transport layer, an electron injection layer, an interlayer, a hole-blocking layer, and an electron buffer layer in addition to the hole transport layer, the light-emitting layer, the hole auxiliary layer, the electron-blocking layer, and the light-emitting auxiliary layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may comprise the amine-based compound, e.g., an arylamine-based compound and a styrylarylamine-based compound, etc., as a hole injection material, a hole transport material, a hole auxiliary material, a light-emitting material, a light-emitting auxiliary material, or an electron-blocking material. Also, the electron transport layer, the electron injection layer, the electron buffer layer, or the hole-blocking layer may comprise the azine-based compound as an electron transport material, an electron injection material, an electron buffer material, or a hole-blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent compound according to one embodiment may be used as light-emitting materials for a white organic light-emitting device. The white organic light-emitting device has been suggested to have various structures such as a parallel side-by-side arrangement method, a stacking arrangement method, or a CCM (color conversion material) method, etc. according to the arrangement of R (red), G (green), YG (yellowish green), or B (blue) light-emitting units. In addition, the organic electroluminescent compound according to one embodiment may also be applied to the organic electroluminescent device comprising a QD (quantum dot).

According to one embodiment, any one of the first electrode and the second electrode may be an anode and the other may be a cathode. Wherein, the first electrode and the second electrode may each be formed of a transparent conductive material, or of a semi-transparent or reflective conductive material. The organic electroluminescent device may be a top emission type, a bottom emission type, or a both-sides emission type depending on the kinds of the material forming the first electrode and the second electrode.

A hole injection layer, a hole transport layer, an electron-blocking layer, or a combination thereof can be used between the anode and the light-emitting layer. The hole injection layer may be multi-layers in order to lower the hole injection barrier (or hole injection voltage) from the anode to the hole transport layer or the electron-blocking layer, wherein each of the multi-layers may use two compounds simultaneously. Also, the hole injection layer may be doped with a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent light-emitting leakage. The hole transport layer or the electron-blocking layer may be multi-layers, wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped with an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ (1≤X≤2), AlOₓ (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare earth metals, and mixtures thereof. Also, a reductive dopant layer may be employed as a charge generation layer to prepare an organic electroluminescent device which has two or more light-emitting layers and emits white light.

According to another embodiment of the present disclosure, an organic electroluminescent device is provided, comprising: a first electrode; a second electrode opposing the first electrode; two or more light-emitting units disposed between the first electrode and the second electrode, each of which includes at least one light-emitting layer; and an n-type charge generation layer disposed between each of the light-emitting units. At least one of the light-emitting units includes a first light-emitting layer and a second light-emitting layer which are adjacent to each other, and the n-type charge generation layer includes at least one compound having a skeleton of phenanthroline, quinazoline, quinoxaline, terpyridine, and phenanthrooxazole.

According to one embodiment, the organic electroluminescent device further includes an electron transport layer positioned between the light-emitting unit and the second electrode, and the electron transport layer may include an organic electroluminescent compound represented by the following Formula 3.

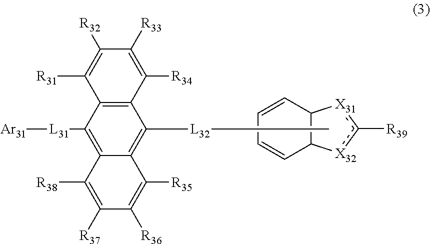

In Formula 3,
R₃₁ to R₃₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring;
R₃₉ represents hydrogen or a substituted or unsubstituted (C₁-C₃₀)alkyl;
L₃₁ and L₃₂ each independently represent a single bond or a substituted or unsubstituted (C₆-C₃₀)arylene;
Ar₃₁ represents a substituted or unsubstituted (C₆-C₃₀)aryl or a substituted or unsubstituted (3- to 30-membered)heteroaryl;
X₃₁ and X₃₂ each independently represent NR₄₀; and
R₄₀ is a site linked to L₃₂, or hydrogen, deuterium, or a substituted or unsubstituted (C₁-C₃₀)alkyl.

In one embodiment, the first light-emitting layer may comprise a compound having a condensed ring including four or more rings. For example, the first light-emitting layer may comprise at least one compound having a skeleton of pyrene, benzanthracene, xanthene, chrysene, fluoranthene, triphenylene, benzoxanthene, dibenzochrysene, benzophenanthrene, and phenanthrofuran.

In one embodiment, the second light-emitting layer may include a compound having an anthracene skeleton.

In one embodiment, at least one of the first light-emitting layer and the second light-emitting layer may comprise a compound having at least one deuterium.

In one embodiment, the n-type charge generation layer may include at least one of the following specific compounds, but is not limited thereto.

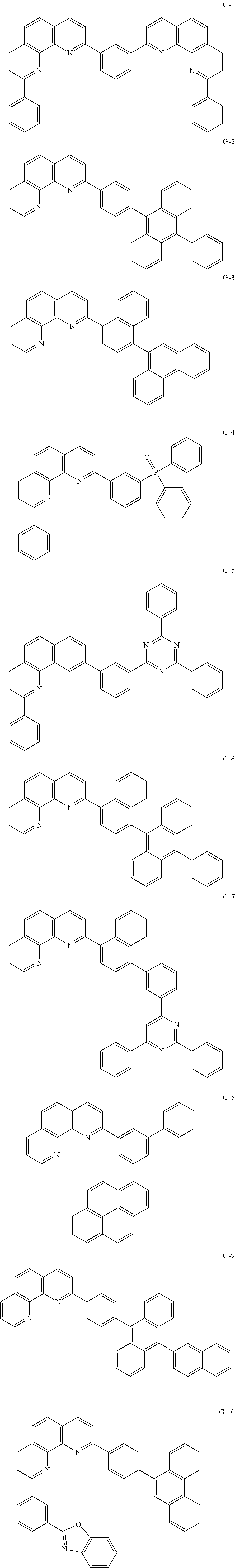

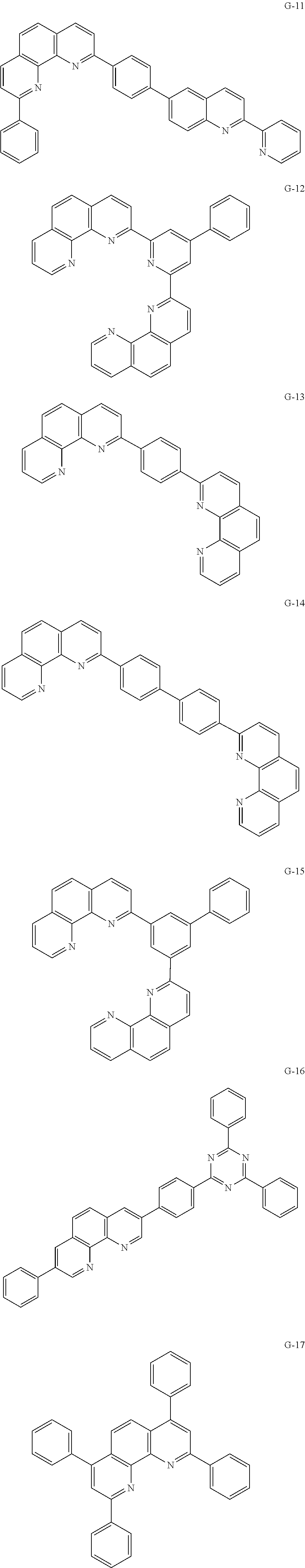

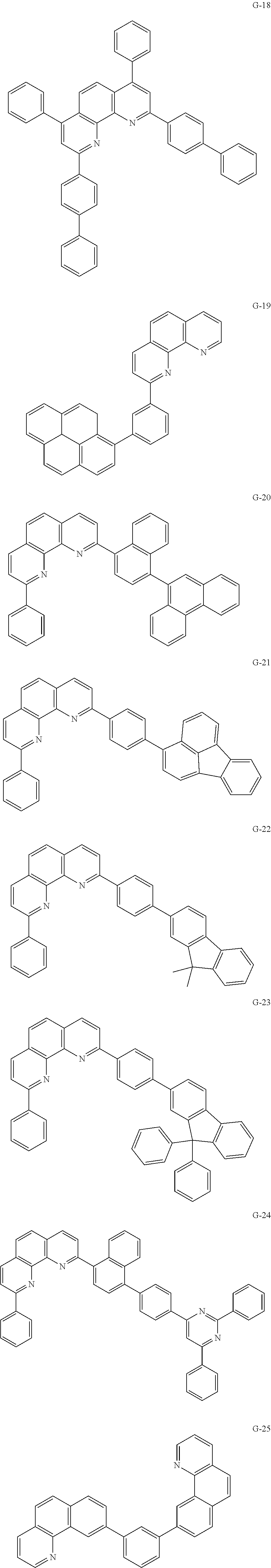

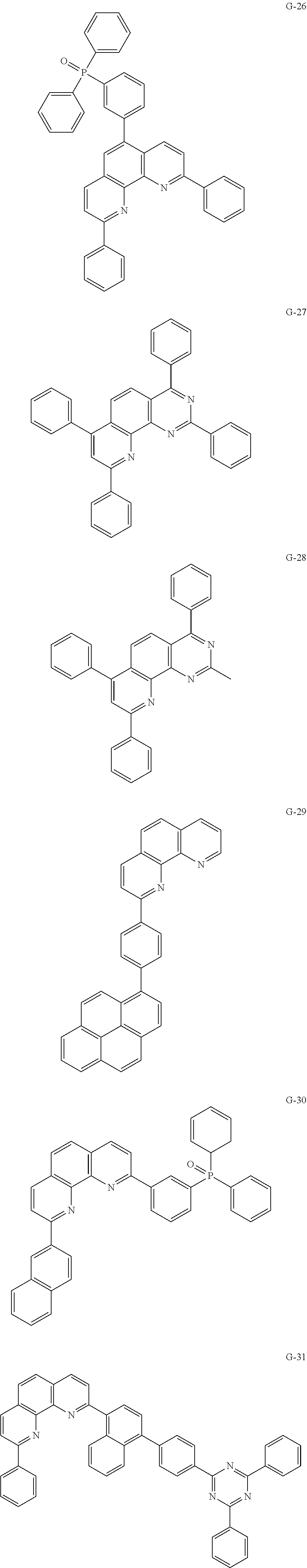 [SMILES: O=P(c1ccccc1)(c1ccccc1)c1cccc(-c2cc3ccc(-c4ccccc4)nc3c3nc(-c4ccccc4)ccc23)c1 ; c1ccc(-c2cc(-c3ccccc3)c3ccc4c(-c5ccccc5)nc(-c5ccccc5)nc4c3n2)cc1 ; Cc1nc(-c2ccccc2)c2ccc3c(-c4ccccc4)cc(-c4ccccc4)nc3c2n1 ; c1cnc2c(c1)ccc1ccc(-c3ccc(-c4ccc5ccc6cccc7ccc4c5c67)cc3)nc12 ; O=P(c1ccccc1)(c1cccc(-c2ccc3ccc4ccc(-c5ccc6ccccc6c5)nc4c3n2)c1)C1C=CC=CC1 ; c1ccc(-c2ccc3ccc4ccc(-c5ccc(-c6ccc(-c7nc(-c8ccccc8)nc(-c8ccccc8)n7)cc6)c6ccccc56)nc4c3n2)cc1]

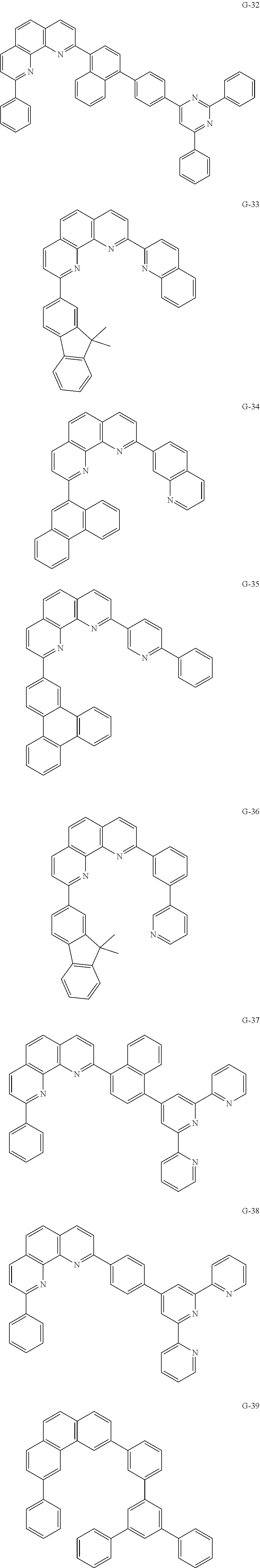

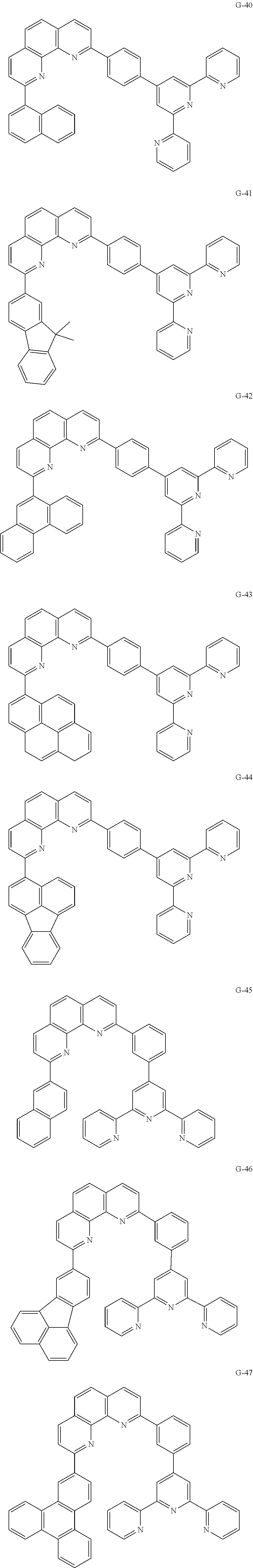

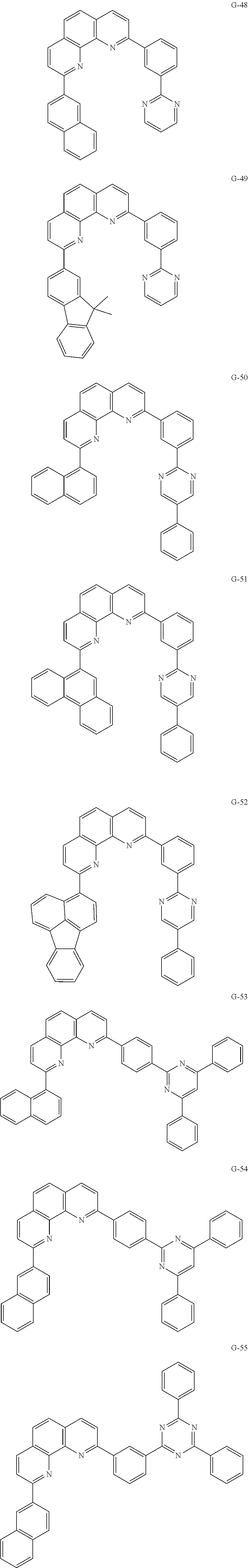

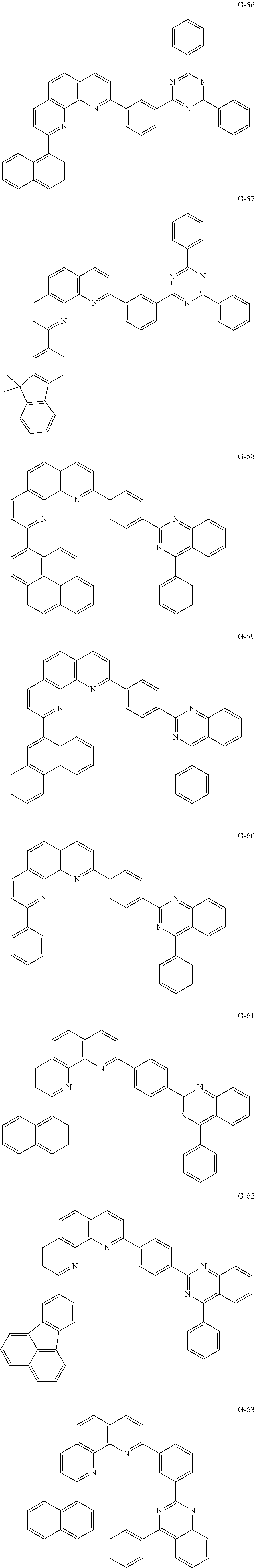

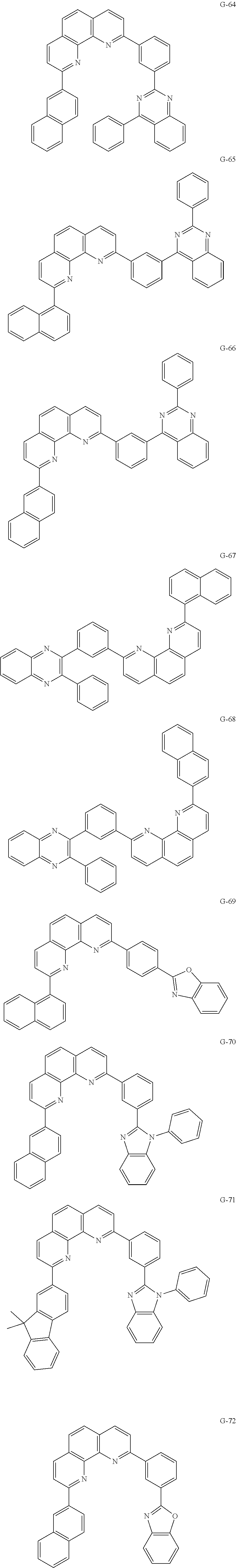

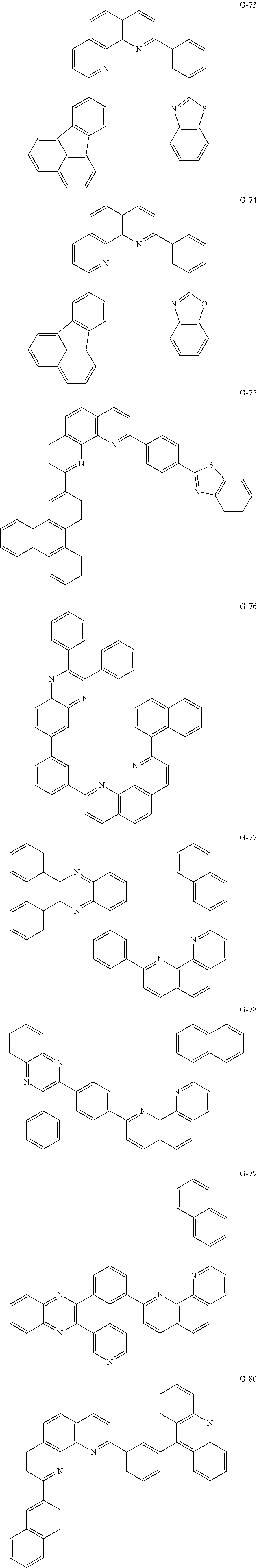

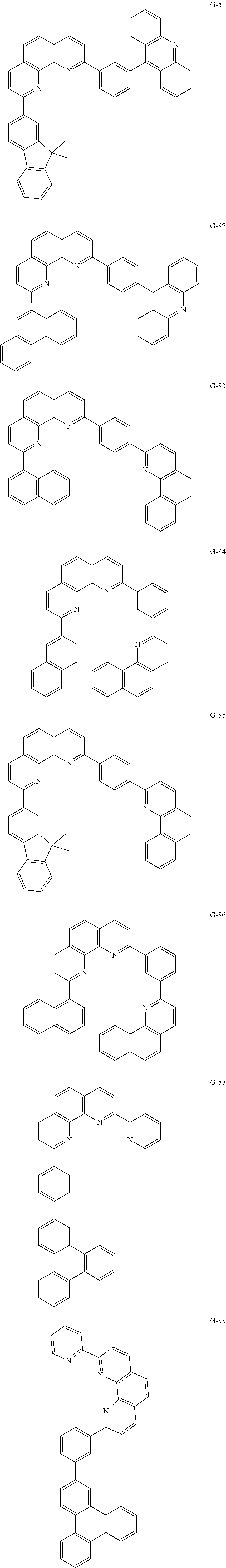

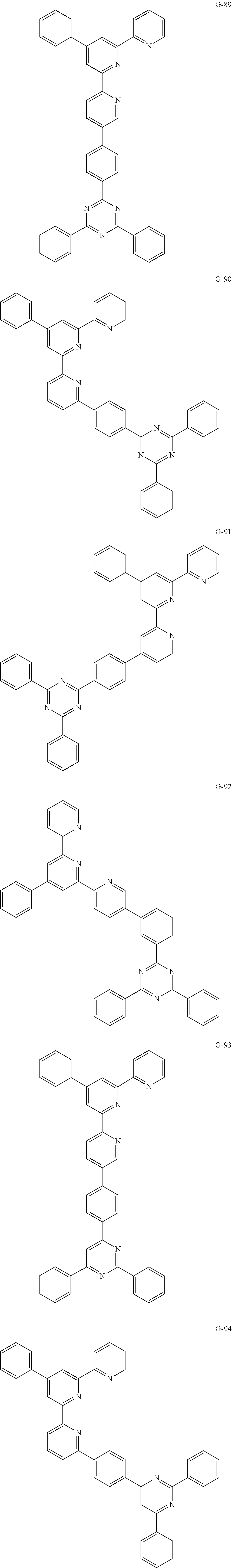

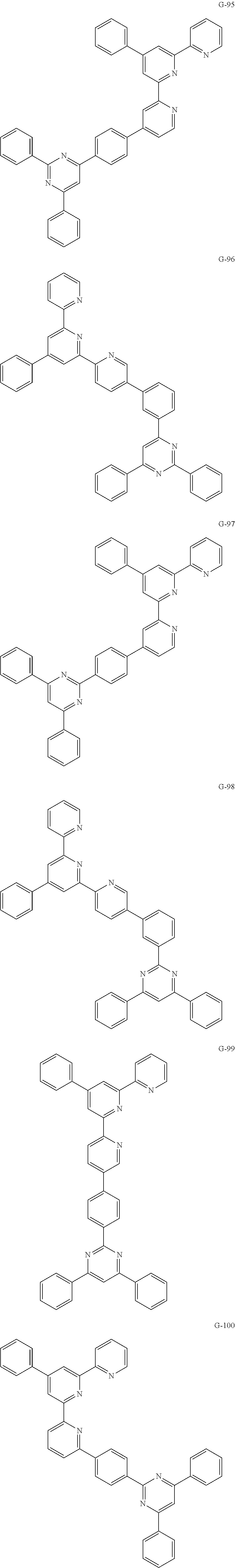

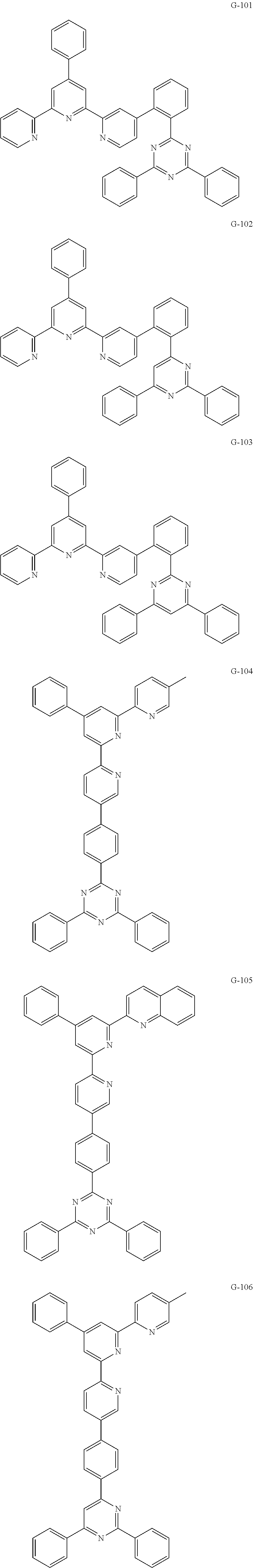

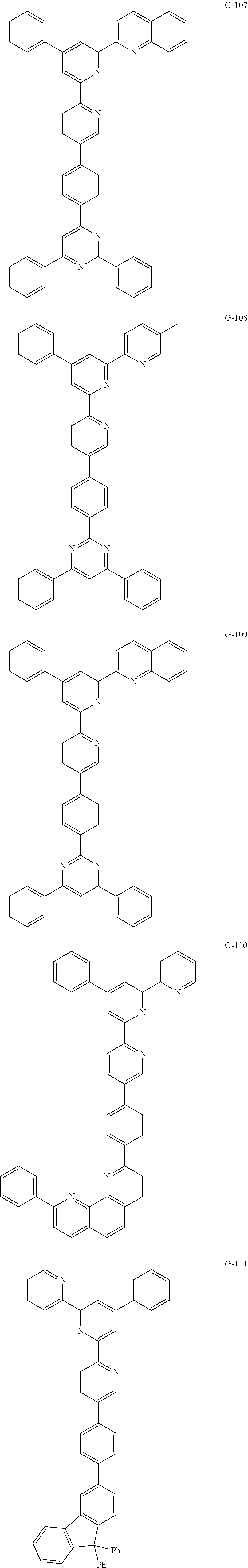

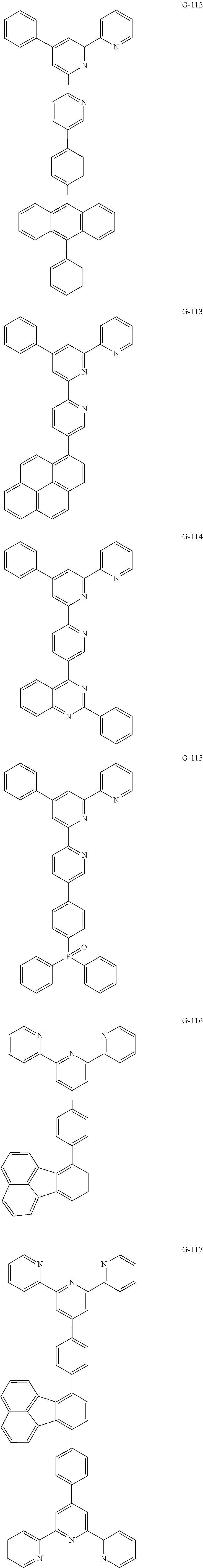

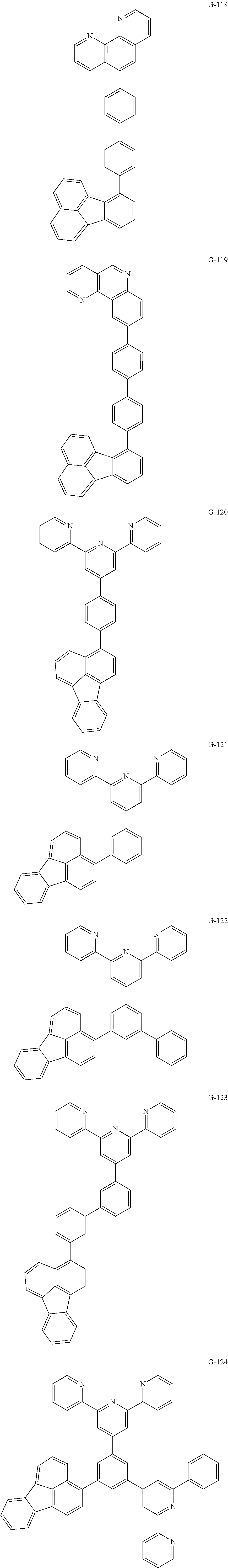

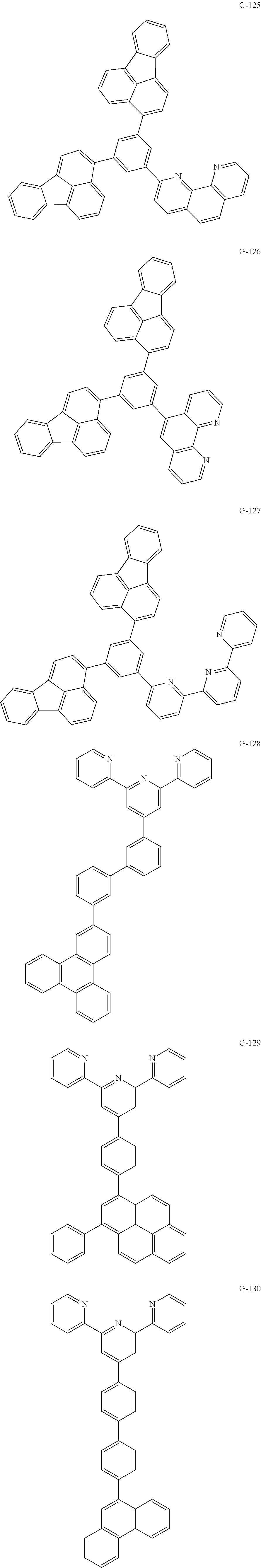

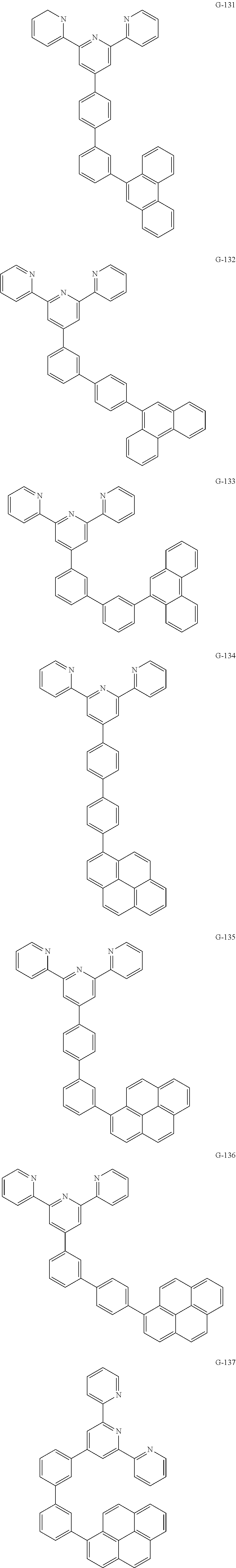

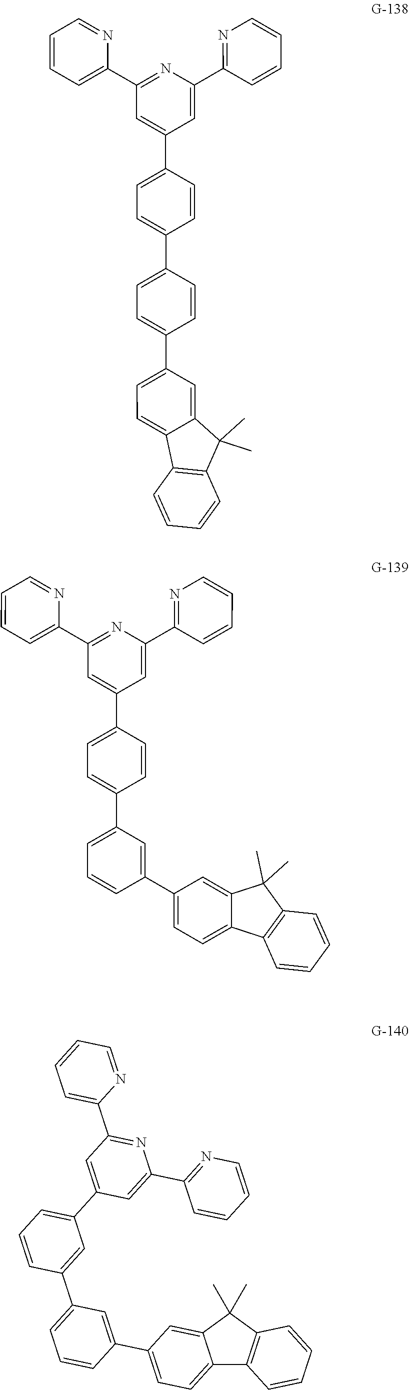 [SMILES: CC1(C)c2ccccc2-c2ccc(-c3ccc(-c4ccc(-c5cc(-c6ccccn6)nc(-c6ccccn6)c5)cc4)cc3)cc21 ; CC1(C)c2ccccc2-c2ccc(-c3cccc(-c4ccc(-c5cc(-c6ccccn6)nc(-c6ccccn6)c5)cc4)c3)cc21 ; CC1(C)c2ccccc2-c2ccc(-c3cccc(-c4cccc(-c5cc(-c6ccccn6)nc(-c6ccccn6)c5)c4)c3)cc21]

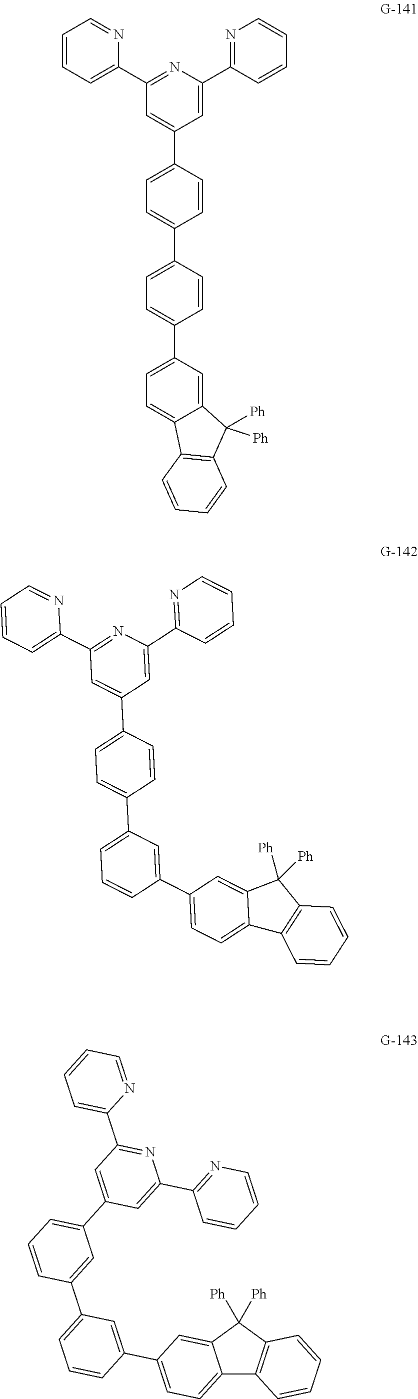 [SMILES: c1ccc(C2(c3ccccc3)c3ccccc3-c3ccc(-c4ccc(-c5ccc(-c6cc(-c7ccccn7)nc(-c7ccccn7)c6)cc5)cc4)cc32)cc1 ; c1ccc(C2(c3ccccc3)c3ccccc3-c3ccc(-c4cccc(-c5ccc(-c6cc(-c7ccccn7)nc(-c7ccccn7)c6)cc5)c4)cc32)cc1 ; c1ccc(C2(c3ccccc3)c3ccccc3-c3ccc(-c4cccc(-c5cccc(-c6cc(-c7ccccn7)nc(-c7ccccn7)c6)c5)c4)cc32)cc1]

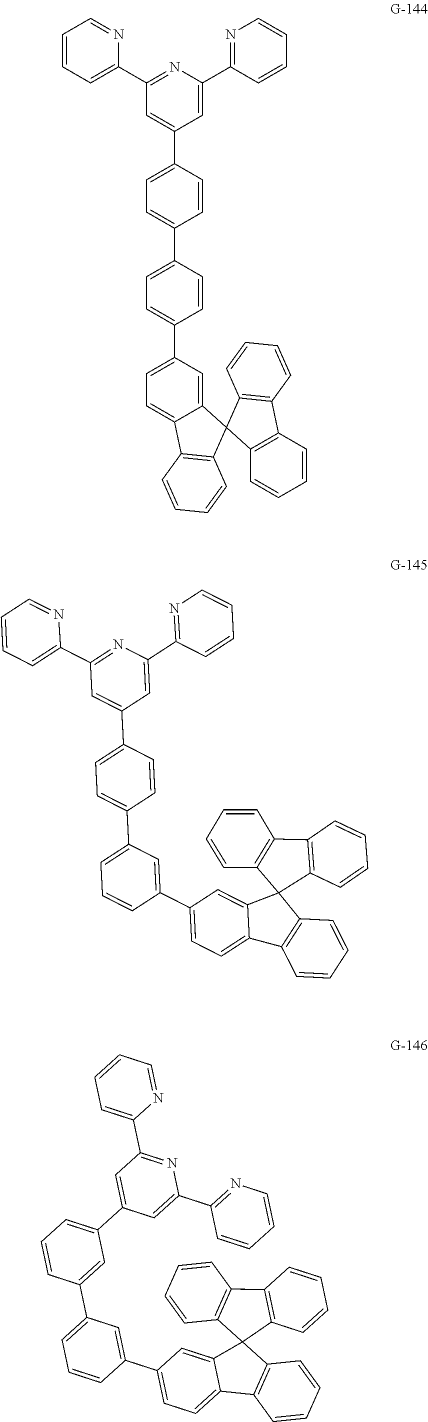 [SMILES: c1ccc(-c2cc(-c3ccc(-c4ccc(-c5ccc6c(c5)C5(c7ccccc7-c7ccccc75)c5ccccc5-6)cc4)cc3)cc(-c3ccccn3)n2)nc1 ; c1ccc(-c2cc(-c3ccc(-c4cccc(-c5ccc6c(c5)C5(c7ccccc7-c7ccccc75)c5ccccc5-6)c4)cc3)cc(-c3ccccn3)n2)nc1 ; c1ccc(-c2cc(-c3cccc(-c4cccc(-c5ccc6c(c5)C5(c7ccccc7-c7ccccc75)c5ccccc5-6)c4)c3)cc(-c3ccccn3)n2)nc1]

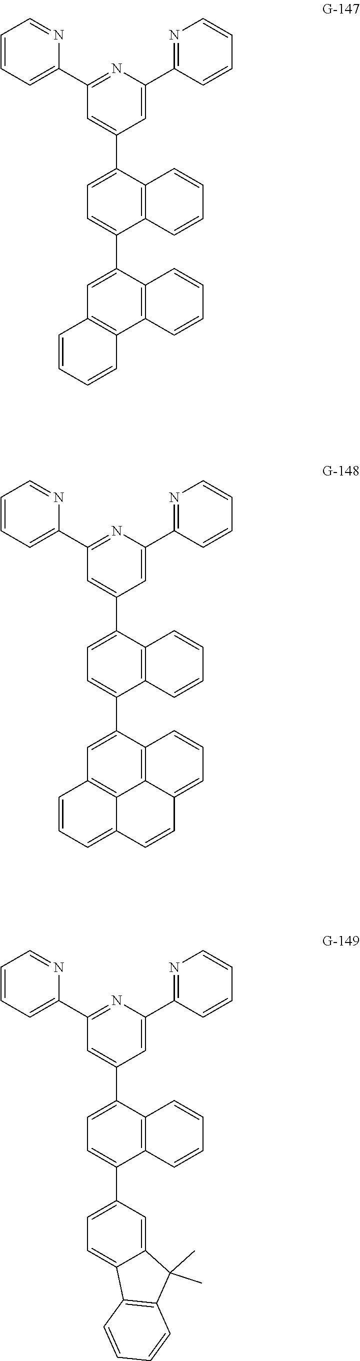 [SMILES: c1ccc(-c2cc(-c3ccc(-c4cc5ccccc5c5ccccc45)c4ccccc34)cc(-c3ccccn3)n2)nc1 ; c1ccc(-c2cc(-c3ccc(-c4cc5cccc6ccc7cccc4c7c65)c4ccccc34)cc(-c3ccccn3)n2)nc1 ; CC1(C)c2ccccc2-c2ccc(-c3ccc(-c4cc(-c5ccccn5)nc(-c5ccccn5)c4)c4ccccc34)cc21]

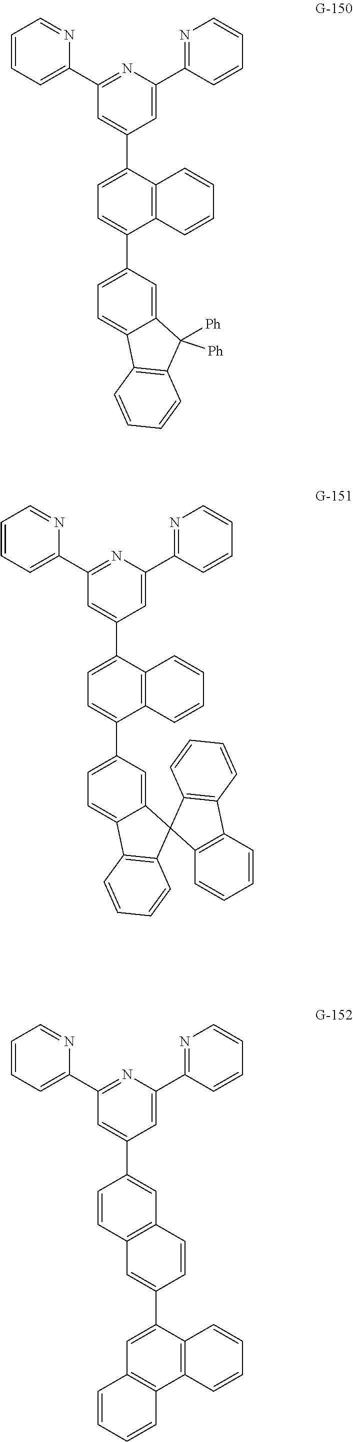 [SMILES: c1ccc(C2(c3ccccc3)c3ccccc3-c3ccc(-c4ccc(-c5cc(-c6ccccn6)nc(-c6ccccn6)c5)c5ccccc45)cc32)cc1 ; c1ccc(-c2cc(-c3ccc(-c4ccc5c(c4)C4(c6ccccc6-c6ccccc64)c4ccccc4-5)c4ccccc34)cc(-c3ccccn3)n2)nc1 ; c1ccc(-c2cc(-c3ccc4cc(-c5cc6ccccc6c6ccccc56)ccc4c3)cc(-c3ccccn3)n2)nc1]

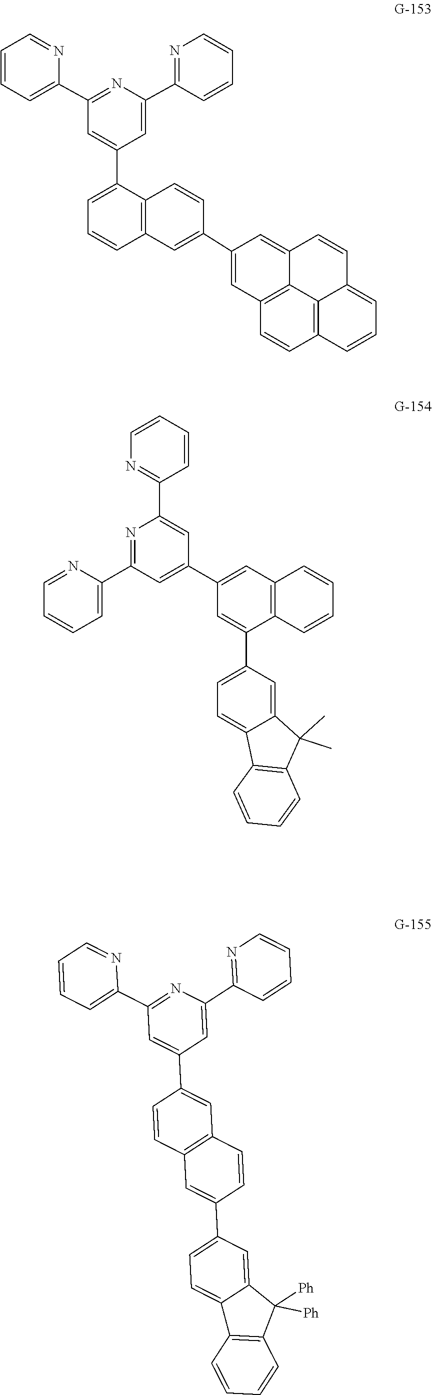 [SMILES: c1ccc(-c2cc(-c3cccc4cc(-c5cc6ccc7cccc8ccc(c5)c6c78)ccc34)cc(-c3ccccn3)n2)nc1 ; CC1(C)c2ccccc2-c2ccc(-c3cc(-c4cc(-c5ccccn5)nc(-c5ccccn5)c4)cc4ccccc34)cc21 ; c1ccc(C2(c3ccccc3)c3ccccc3-c3ccc(-c4ccc5cc(-c6cc(-c7ccccn7)nc(-c7ccccn7)c6)ccc5c4)cc32)cc1]

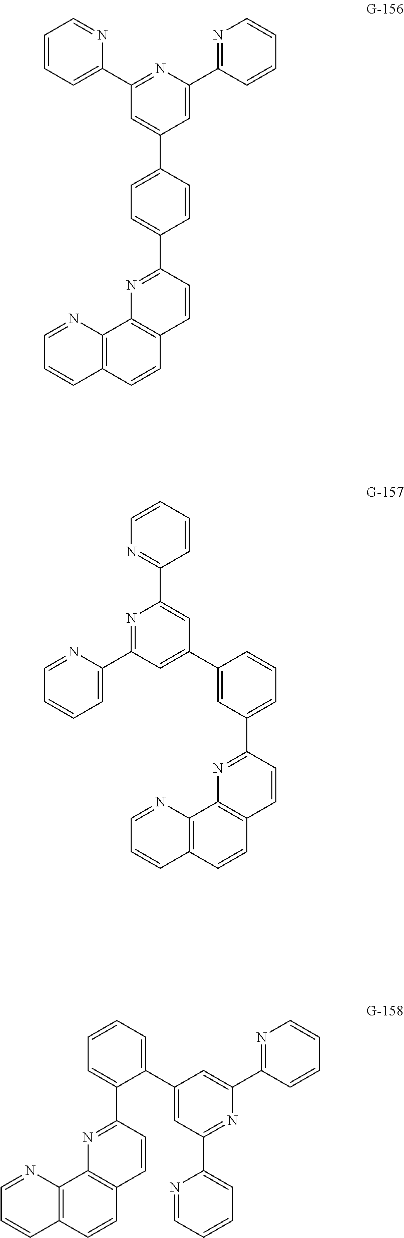 [SMILES: c1ccc(-c2cc(-c3ccc(-c4ccc5ccc6cccnc6c5n4)cc3)cc(-c3ccccn3)n2)nc1 ; c1ccc(-c2cc(-c3cccc(-c4ccc5ccc6cccnc6c5n4)c3)cc(-c3ccccn3)n2)nc1 ; c1ccc(-c2cc(-c3ccccc3-c3ccc4ccc5cccnc5c4n3)cc(-c3ccccn3)n2)nc1]

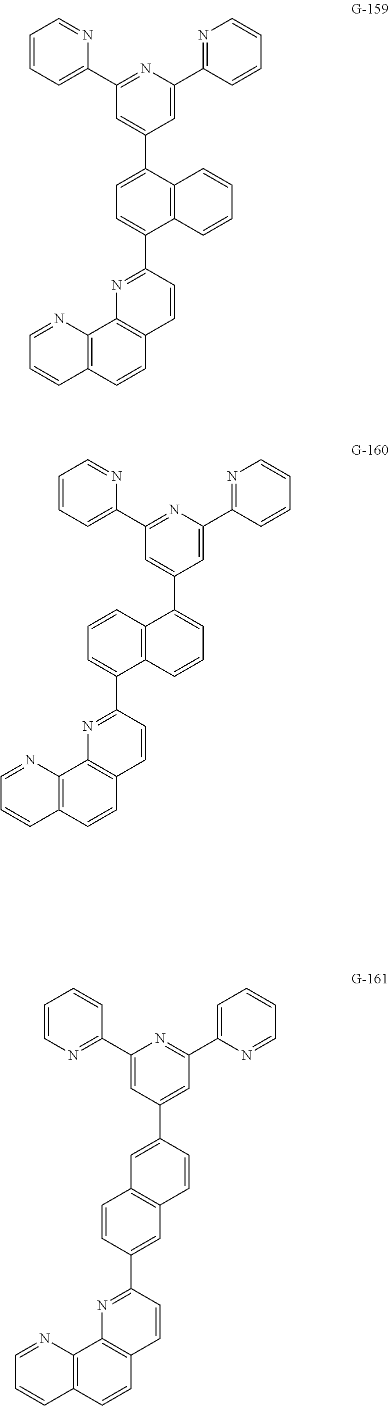 [SMILES: c1ccc(-c2cc(-c3ccc(-c4ccc5ccc6cccnc6c5n4)c4ccccc34)cc(-c3ccccn3)n2)nc1 ; c1ccc(-c2cc(-c3cccc4c(-c5ccc6ccc7cccnc7c6n5)cccc34)cc(-c3ccccn3)n2)nc1 ; c1ccc(-c2cc(-c3ccc4cc(-c5ccc6ccc7cccnc7c6n5)ccc4c3)cc(-c3ccccn3)n2)nc1]

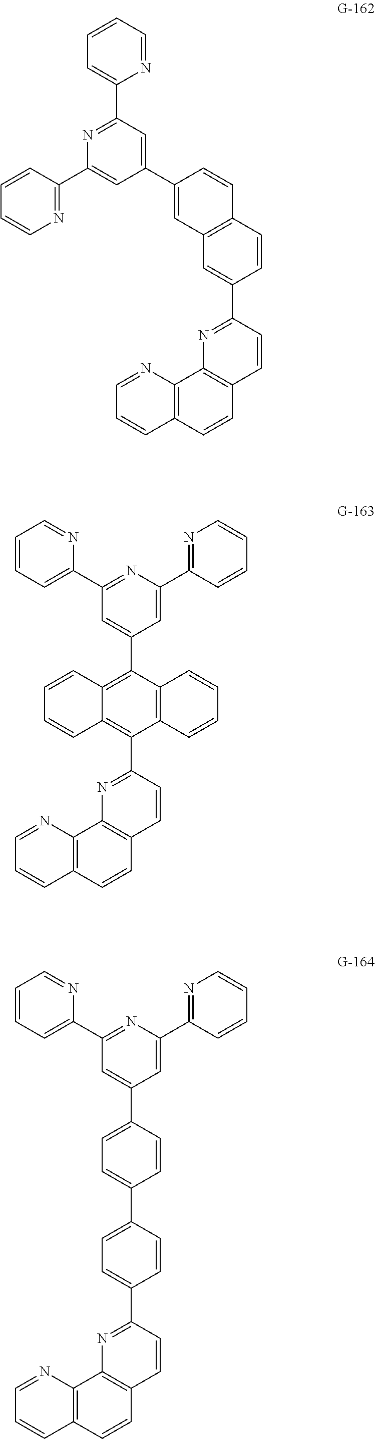 [SMILES: c1ccc(-c2cc(-c3ccc4ccc(-c5ccc6ccc7cccnc7c6n5)cc4c3)cc(-c3ccccn3)n2)nc1 ; c1ccc(-c2cc(-c3c4ccccc4c(-c4ccc5ccc6cccnc6c5n4)c4ccccc34)cc(-c3ccccn3)n2)nc1 ; c1ccc(-c2cc(-c3ccc(-c4ccc(-c5ccc6ccc7cccnc7c6n5)cc4)cc3)cc(-c3ccccn3)n2)nc1]

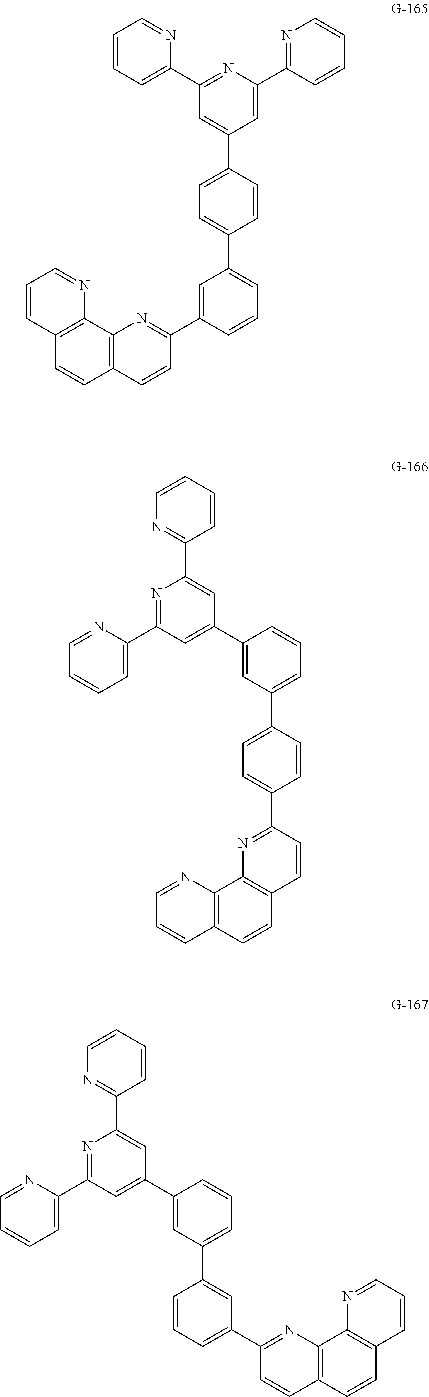 [SMILES: c1ccc(-c2cc(-c3ccc(-c4cccc(-c5ccc6ccc7cccnc7c6n5)c4)cc3)cc(-c3ccccn3)n2)nc1 ; c1ccc(-c2cc(-c3cccc(-c4ccc(-c5ccc6ccc7cccnc7c6n5)cc4)c3)cc(-c3ccccn3)n2)nc1 ; c1ccc(-c2cc(-c3cccc(-c4cccc(-c5ccc6ccc7cccnc7c6n5)c4)c3)cc(-c3ccccn3)n2)nc1]

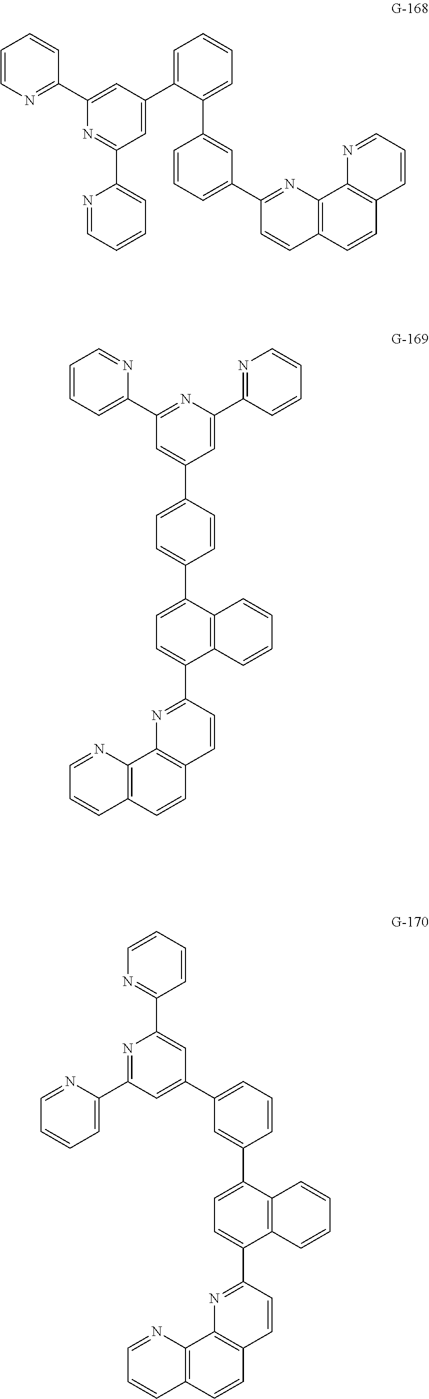 [SMILES: c1ccc(-c2cc(-c3ccccc3-c3cccc(-c4ccc5ccc6cccnc6c5n4)c3)cc(-c3ccccn3)n2)nc1 ; c1ccc(-c2cc(-c3ccc(-c4ccc(-c5ccc6ccc7cccnc7c6n5)c5ccccc45)cc3)cc(-c3ccccn3)n2)nc1 ; c1ccc(-c2cc(-c3cccc(-c4ccc(-c5ccc6ccc7cccnc7c6n5)c5ccccc45)c3)cc(-c3ccccn3)n2)nc1]

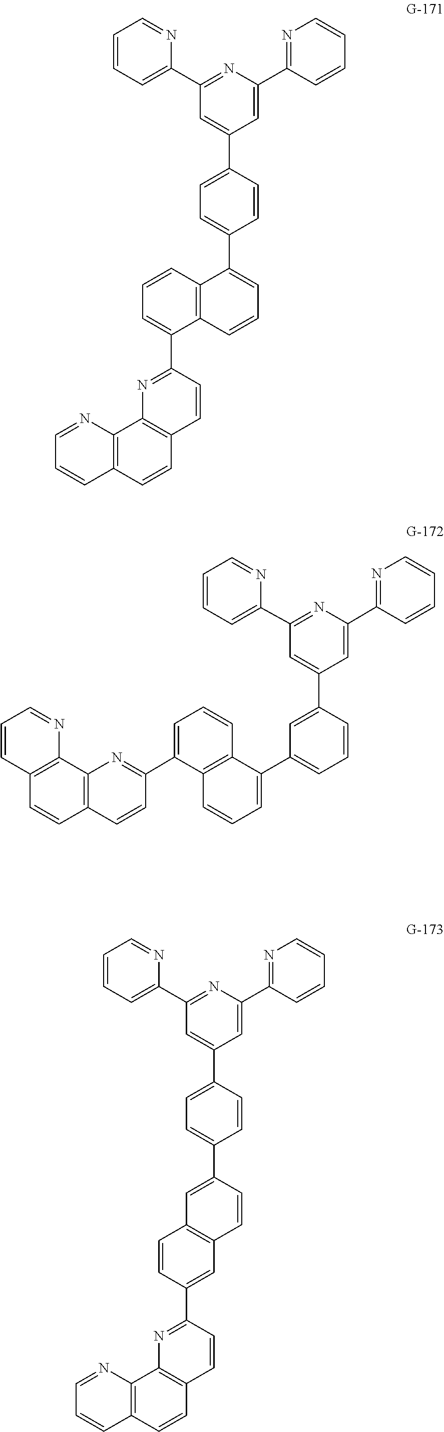 [SMILES: c1ccc(-c2cc(-c3ccc(-c4cccc5c(-c6ccc7ccc8cccnc8c7n6)cccc45)cc3)cc(-c3ccccn3)n2)nc1 ; c1ccc(-c2cc(-c3cccc(-c4cccc5c(-c6ccc7ccc8cccnc8c7n6)cccc45)c3)cc(-c3ccccn3)n2)nc1 ; c1ccc(-c2cc(-c3ccc(-c4ccc5cc(-c6ccc7ccc8cccnc8c7n6)ccc5c4)cc3)cc(-c3ccccn3)n2)nc1]

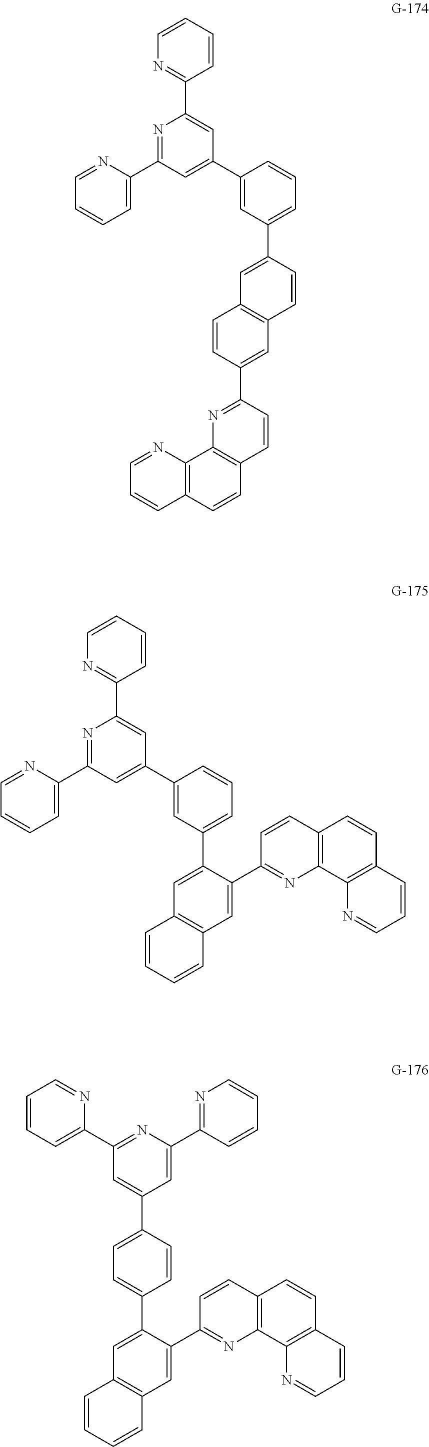 [SMILES: c1ccc(-c2cc(-c3cccc(-c4ccc5cc(-c6ccc7ccc8cccnc8c7n6)ccc5c4)c3)cc(-c3ccccn3)n2)nc1 ; c1ccc(-c2cc(-c3cccc(-c4cc5ccccc5cc4-c4ccc5ccc6cccnc6c5n4)c3)cc(-c3ccccn3)n2)nc1 ; c1ccc(-c2cc(-c3ccc(-c4cc5ccccc5cc4-c4ccc5ccc6cccnc6c5n4)cc3)cc(-c3ccccn3)n2)nc1]

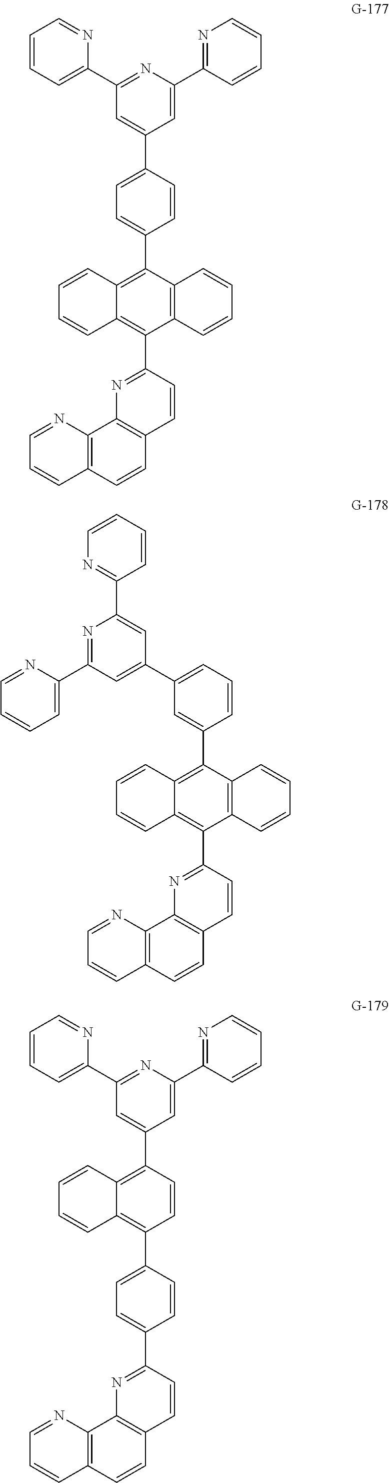

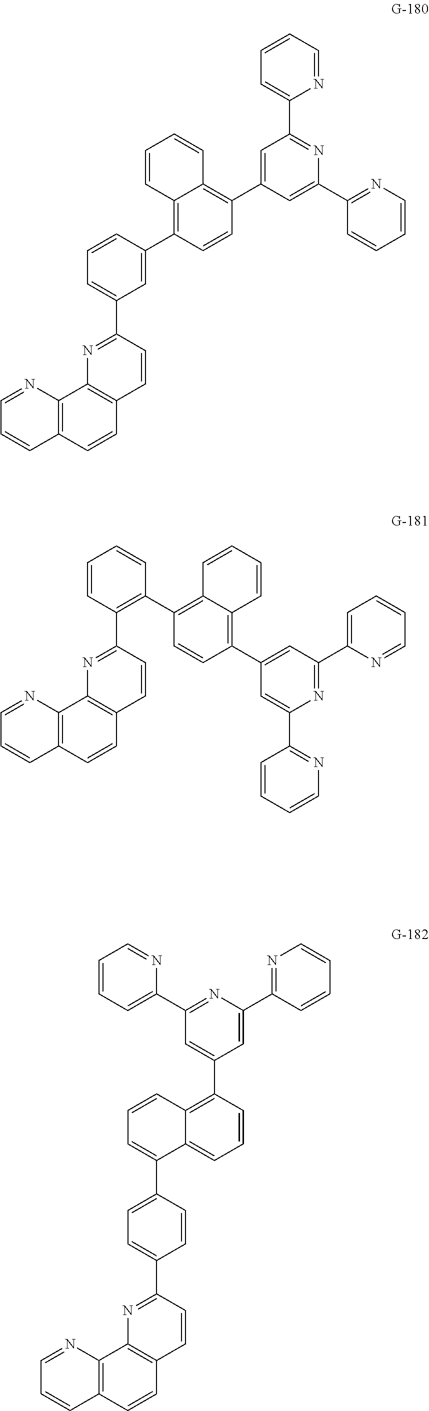

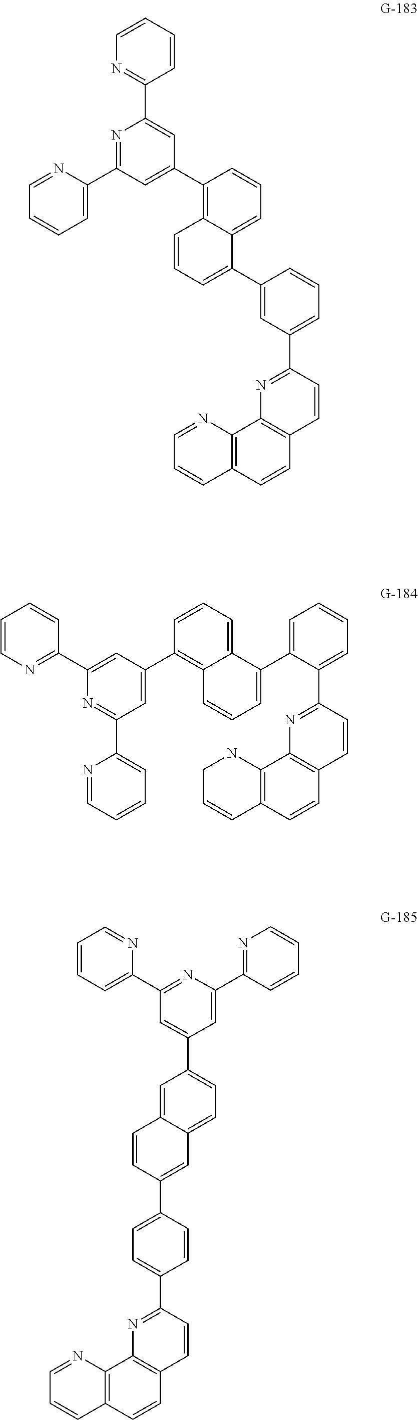

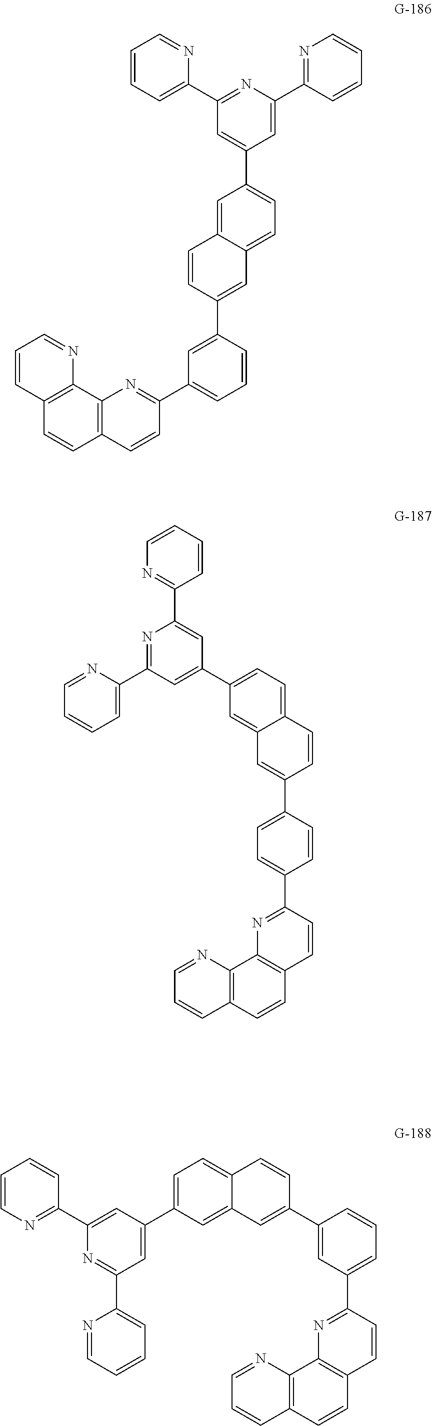 [SMILES: c1ccc(-c2cc(-c3ccc4cc(-c5cccc(-c6ccc7ccc8cccnc8c7n6)c5)ccc4c3)cc(-c3ccccn3)n2)nc1 ; c1ccc(-c2cc(-c3ccc4ccc(-c5ccc(-c6ccc7ccc8cccnc8c7n6)cc5)cc4c3)cc(-c3ccccn3)n2)nc1 ; c1ccc(-c2cc(-c3ccc4ccc(-c5cccc(-c6ccc7ccc8cccnc8c7n6)c5)cc4c3)cc(-c3ccccn3)n2)nc1]

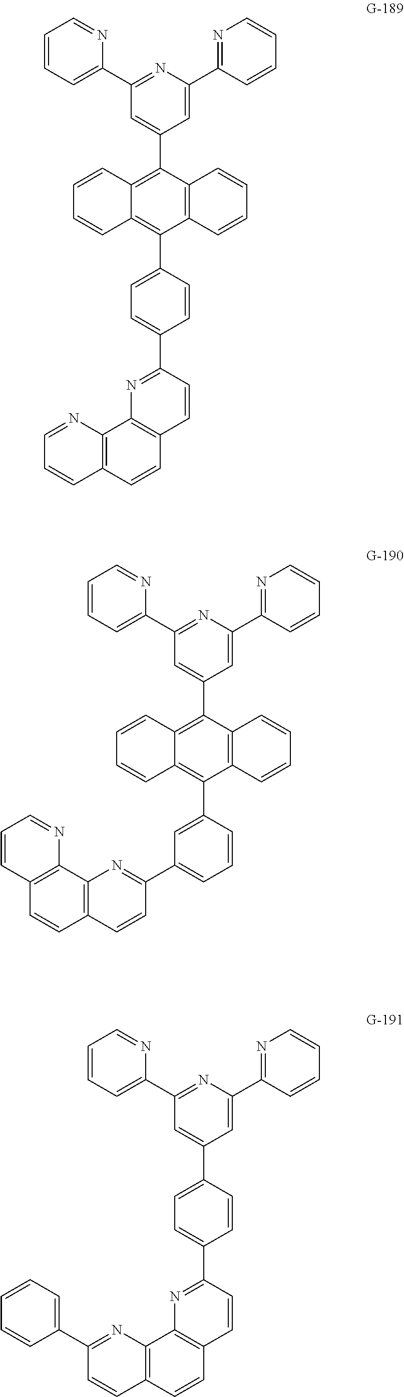 [SMILES: c1ccc(-c2cc(-c3c4ccccc4c(-c4ccc(-c5ccc6ccc7cccnc7c6n5)cc4)c4ccccc34)cc(-c3ccccn3)n2)nc1 ; c1ccc(-c2cc(-c3c4ccccc4c(-c4cccc(-c5ccc6ccc7cccnc7c6n5)c4)c4ccccc34)cc(-c3ccccn3)n2)nc1 ; c1ccc(-c2ccc3ccc4ccc(-c5ccc(-c6cc(-c7ccccn7)nc(-c7ccccn7)c6)cc5)nc4c3n2)cc1]

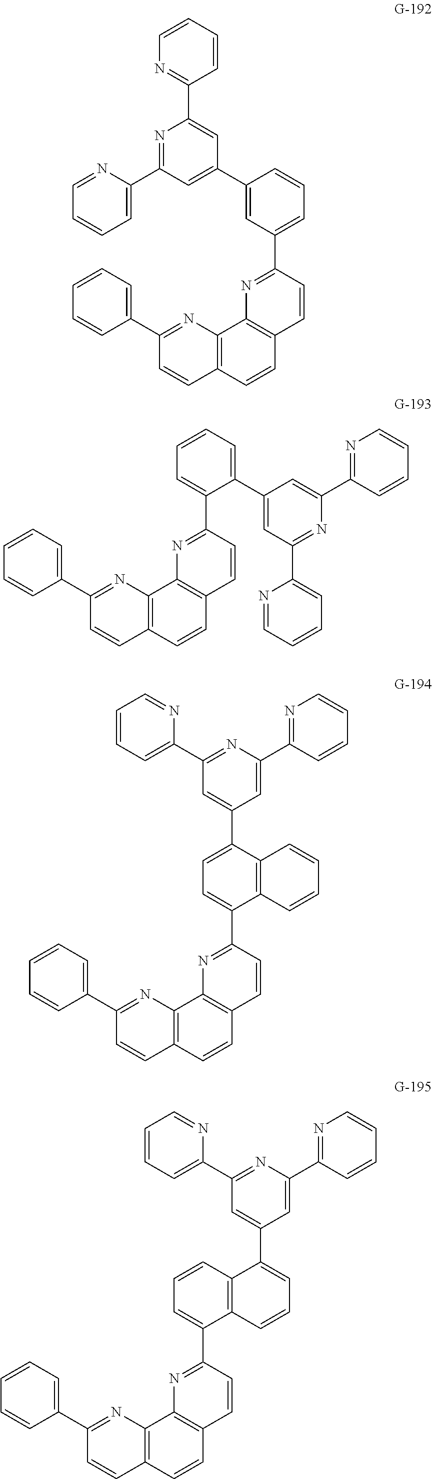 [SMILES: c1ccc(-c2ccc3ccc4ccc(-c5cccc(-c6cc(-c7ccccn7)nc(-c7ccccn7)c6)c5)nc4c3n2)cc1 ; c1ccc(-c2ccc3ccc4ccc(-c5ccccc5-c5cc(-c6ccccn6)nc(-c6ccccn6)c5)nc4c3n2)cc1 ; c1ccc(-c2ccc3ccc4ccc(-c5ccc(-c6cc(-c7ccccn7)nc(-c7ccccn7)c6)c6ccccc56)nc4c3n2)cc1 ; c1ccc(-c2ccc3ccc4ccc(-c5cccc6c(-c7cc(-c8ccccn8)nc(-c8ccccn8)c7)cccc56)nc4c3n2)cc1]

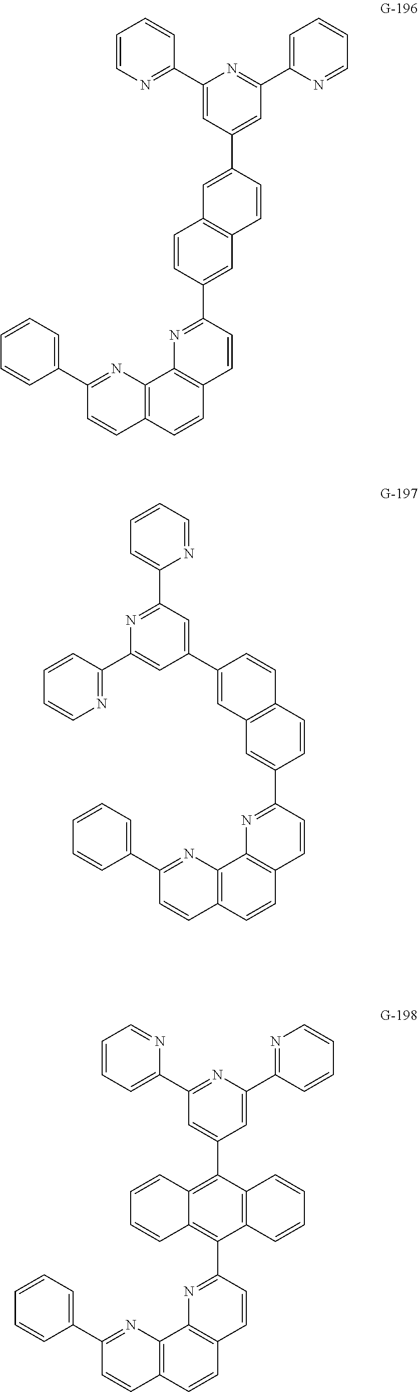 [SMILES: c1ccc(-c2ccc3ccc4ccc(-c5ccc6cc(-c7cc(-c8ccccn8)nc(-c8ccccn8)c7)ccc6c5)nc4c3n2)cc1 ; c1ccc(-c2ccc3ccc4ccc(-c5ccc6ccc(-c7cc(-c8ccccn8)nc(-c8ccccn8)c7)cc6c5)nc4c3n2)cc1 ; c1ccc(-c2ccc3ccc4ccc(-c5c6ccccc6c(-c6cc(-c7ccccn7)nc(-c7ccccn7)c6)c6ccccc56)nc4c3n2)cc1]

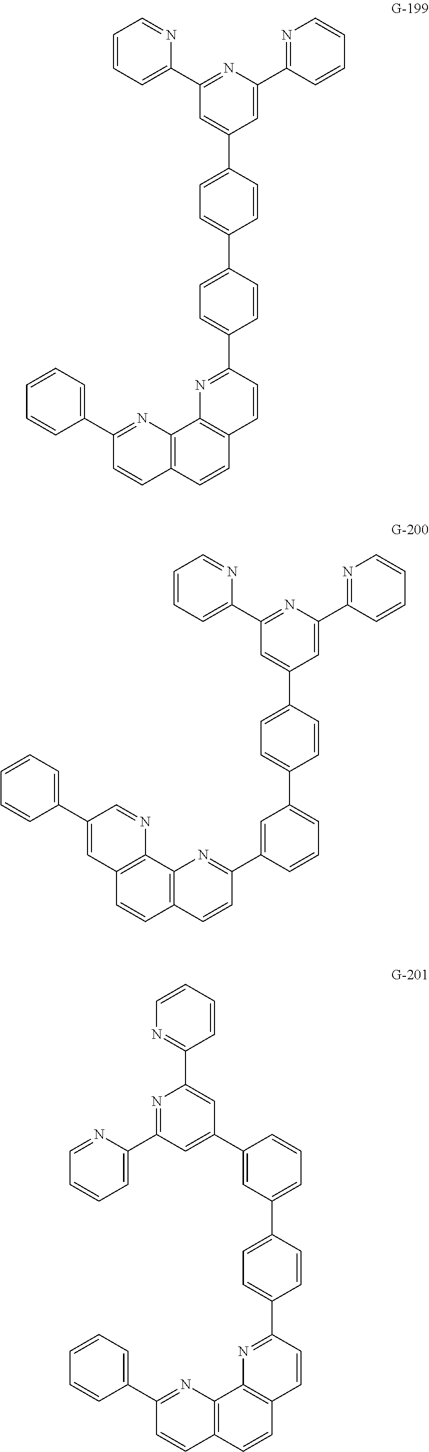

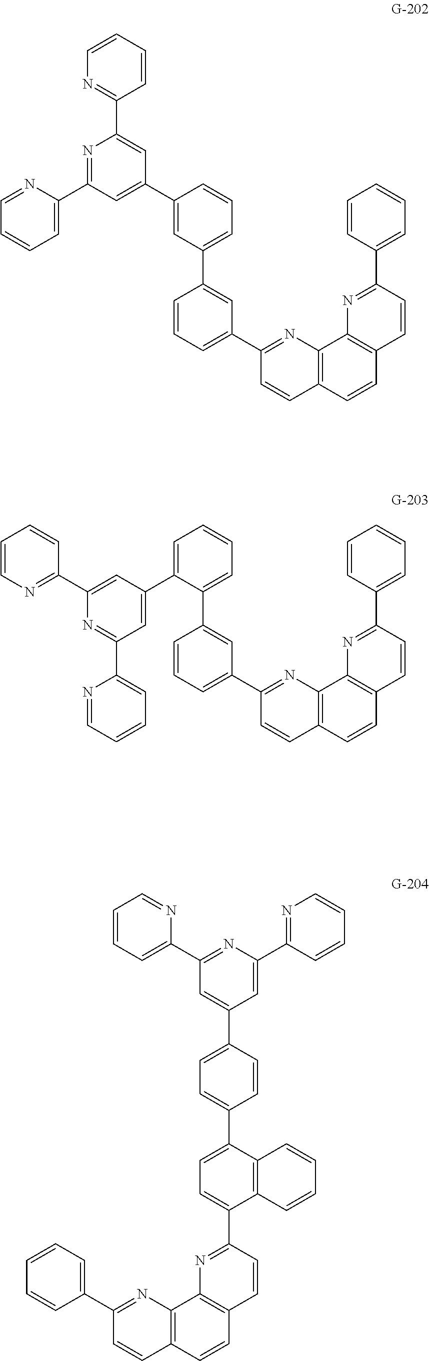 [SMILES: c1ccc(-c2ccc3ccc4ccc(-c5cccc(-c6cccc(-c7cc(-c8ccccn8)nc(-c8ccccn8)c7)c6)c5)nc4c3n2)cc1 ; c1ccc(-c2ccc3ccc4ccc(-c5cccc(-c6ccccc6-c6cc(-c7ccccn7)nc(-c7ccccn7)c6)c5)nc4c3n2)cc1 ; c1ccc(-c2ccc3ccc4ccc(-c5ccc(-c6ccc(-c7cc(-c8ccccn8)nc(-c8ccccn8)c7)cc6)c6ccccc56)nc4c3n2)cc1]

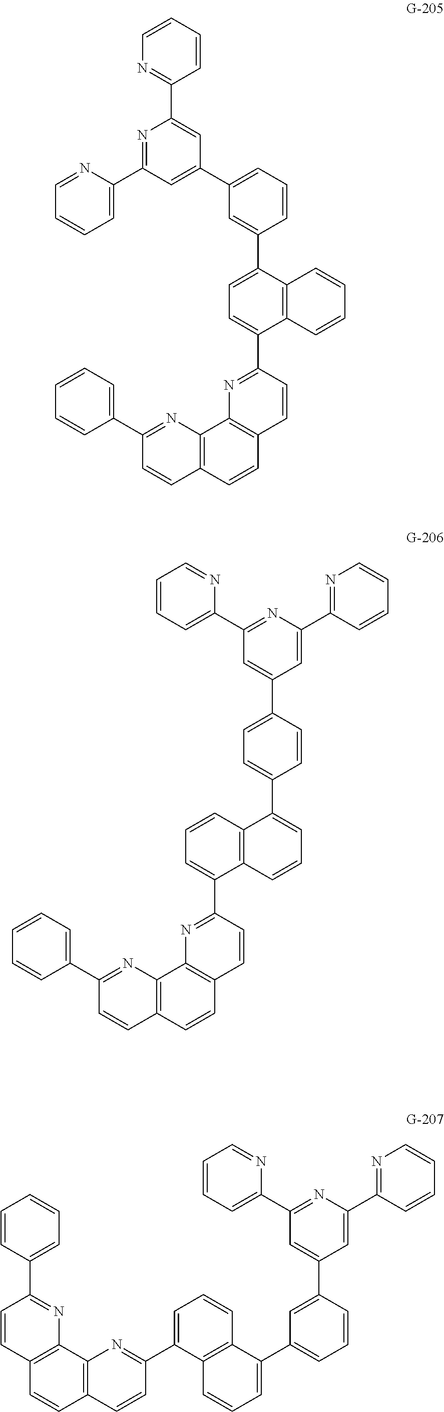 [SMILES: c1ccc(-c2ccc3ccc4ccc(-c5ccc(-c6cccc(-c7cc(-c8ccccn8)nc(-c8ccccn8)c7)c6)c6ccccc56)nc4c3n2)cc1 ; c1ccc(-c2ccc3ccc4ccc(-c5cccc6c(-c7ccc(-c8cc(-c9ccccn9)nc(-c9ccccn9)c8)cc7)cccc56)nc4c3n2)cc1 ; c1ccc(-c2ccc3ccc4ccc(-c5cccc6c(-c7cccc(-c8cc(-c9ccccn9)nc(-c9ccccn9)c8)c7)cccc56)nc4c3n2)cc1]

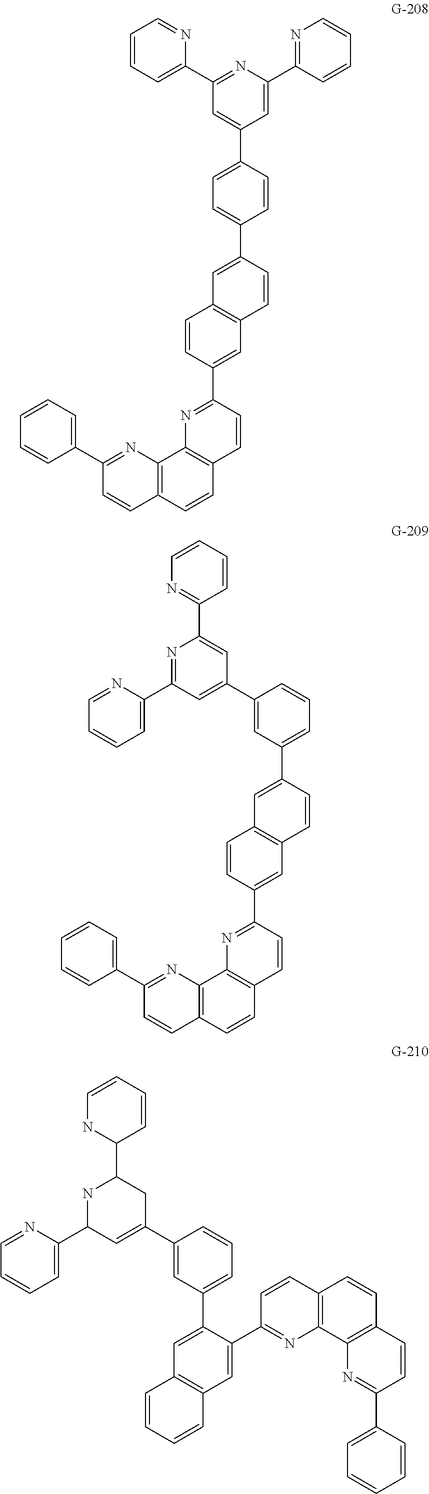

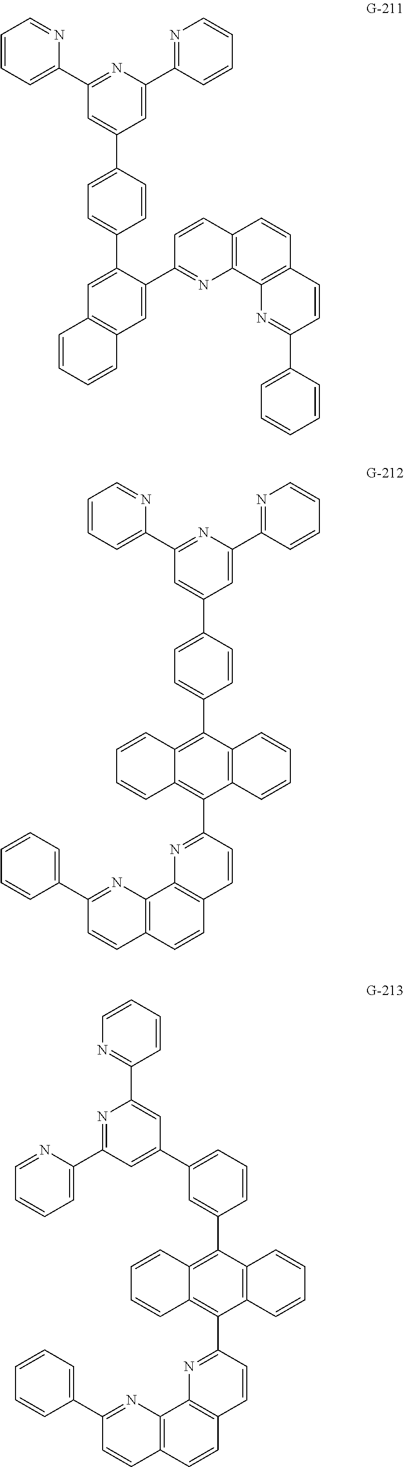 [SMILES: c1ccc(-c2ccc3ccc4ccc(-c5cc6ccccc6cc5-c5ccc(-c6cc(-c7ccccn7)nc(-c7ccccn7)c6)cc5)nc4c3n2)cc1 ; c1ccc(-c2ccc3ccc4ccc(-c5c6ccccc6c(-c6ccc(-c7cc(-c8ccccn8)nc(-c8ccccn8)c7)cc6)c6ccccc56)nc4c3n2)cc1 ; c1ccc(-c2ccc3ccc4ccc(-c5c6ccccc6c(-c6cccc(-c7cc(-c8ccccn8)nc(-c8ccccn8)c7)c6)c6ccccc56)nc4c3n2)cc1]

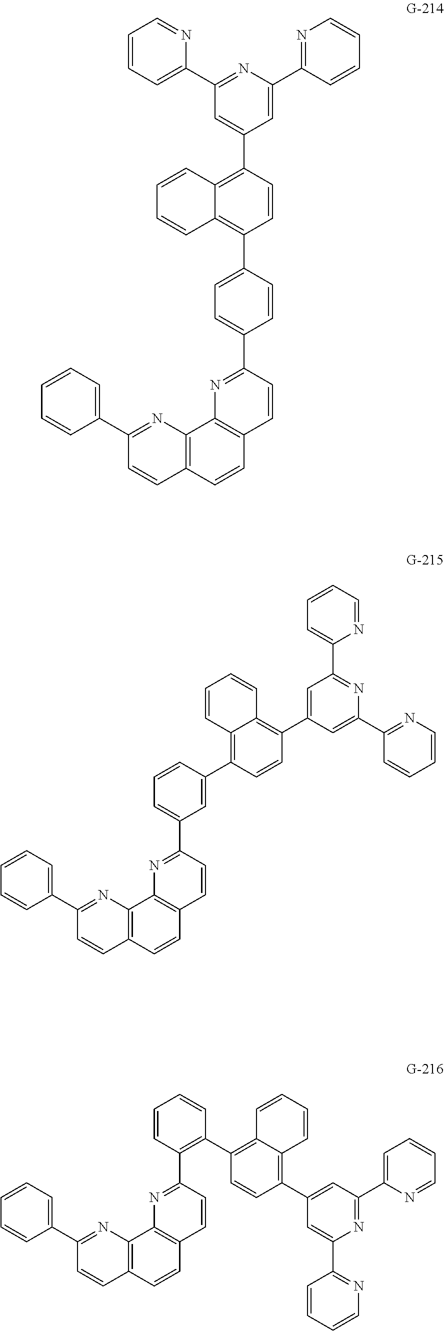 [SMILES: c1ccc(-c2ccc3ccc4ccc(-c5ccc(-c6ccc(-c7cc(-c8ccccn8)nc(-c8ccccn8)c7)c7ccccc67)cc5)nc4c3n2)cc1 ; c1ccc(-c2ccc3ccc4ccc(-c5cccc(-c6ccc(-c7cc(-c8ccccn8)nc(-c8ccccn8)c7)c7ccccc67)c5)nc4c3n2)cc1 ; c1ccc(-c2ccc3ccc4ccc(-c5ccccc5-c5ccc(-c6cc(-c7ccccn7)nc(-c7ccccn7)c6)c6ccccc56)nc4c3n2)cc1]

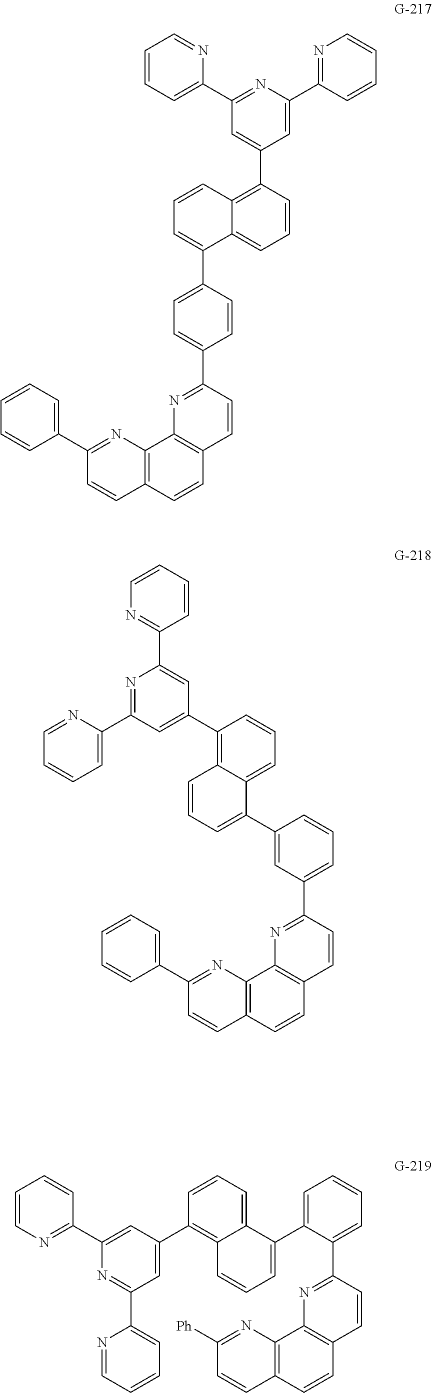 [SMILES: c1ccc(-c2ccc3ccc4ccc(-c5ccc(-c6cccc7c(-c8cc(-c9ccccn9)nc(-c9ccccn9)c8)cccc67)cc5)nc4c3n2)cc1 ; c1ccc(-c2ccc3ccc4ccc(-c5cccc(-c6cccc7c(-c8cc(-c9ccccn9)nc(-c9ccccn9)c8)cccc67)c5)nc4c3n2)cc1 ; c1ccc(-c2ccc3ccc4ccc(-c5ccccc5-c5cccc6c(-c7cc(-c8ccccn8)nc(-c8ccccn8)c7)cccc56)nc4c3n2)cc1]

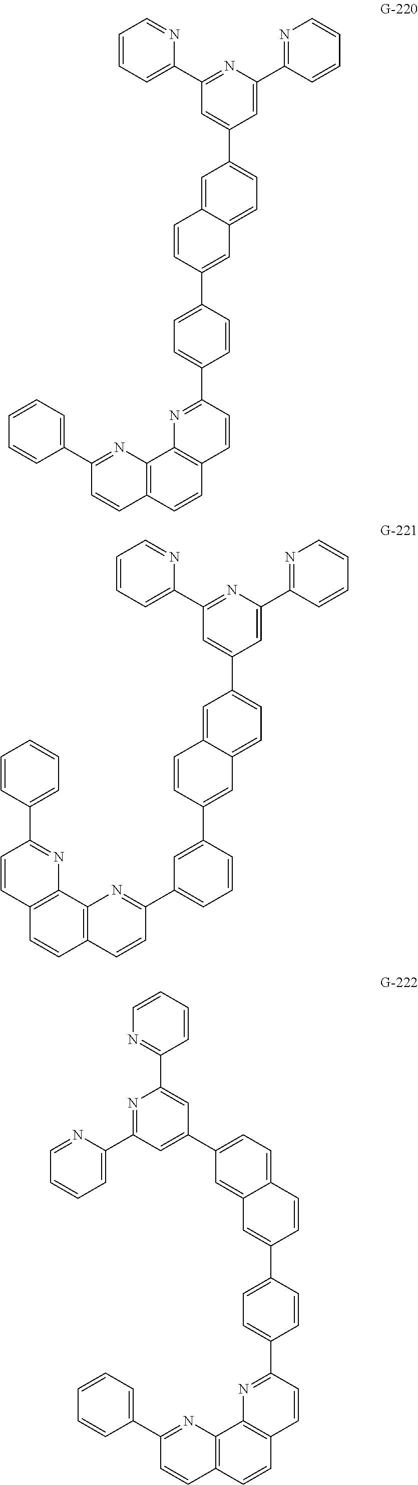

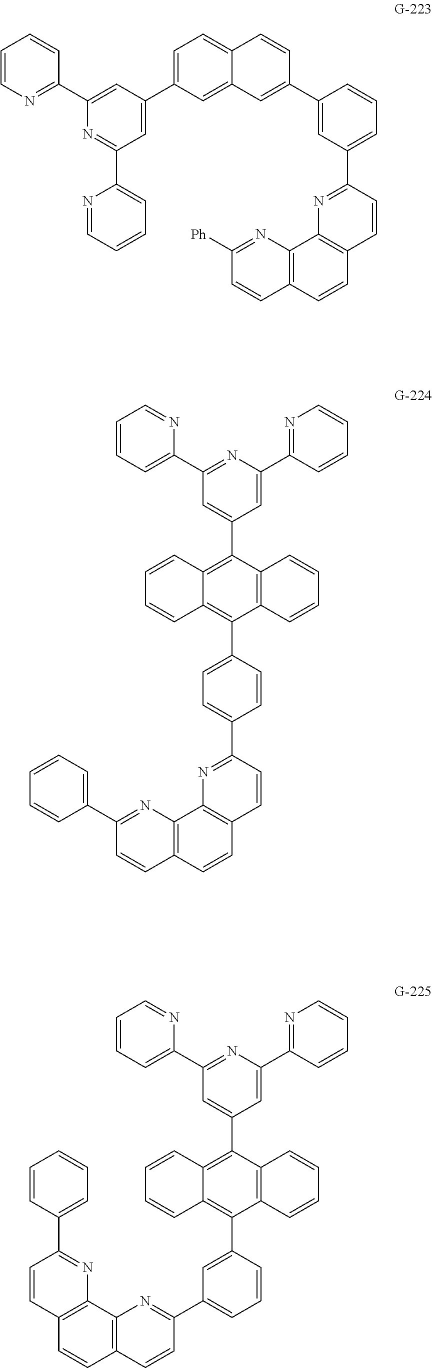 [SMILES: c1ccc(-c2ccc3ccc4ccc(-c5cccc(-c6ccc7ccc(-c8cc(-c9ccccn9)nc(-c9ccccn9)c8)cc7c6)c5)nc4c3n2)cc1 ; c1ccc(-c2ccc3ccc4ccc(-c5ccc(-c6c7ccccc7c(-c7cc(-c8ccccn8)nc(-c8ccccn8)c7)c7ccccc67)cc5)nc4c3n2)cc1 ; c1ccc(-c2ccc3ccc4ccc(-c5cccc(-c6c7ccccc7c(-c7cc(-c8ccccn8)nc(-c8ccccn8)c7)c7ccccc67)c5)nc4c3n2)cc1]

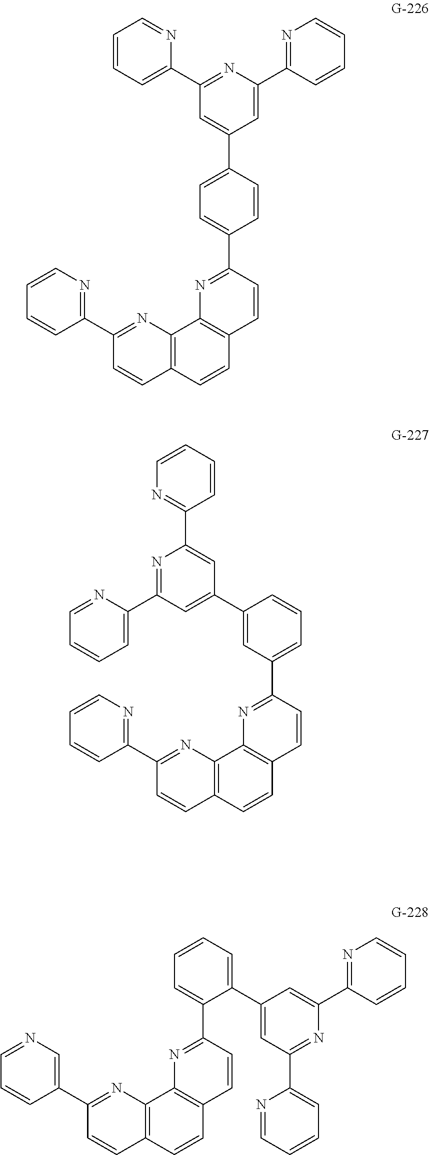 [SMILES: c1ccc(-c2cc(-c3ccc(-c4ccc5ccc6ccc(-c7ccccn7)nc6c5n4)cc3)cc(-c3ccccn3)n2)nc1 ; c1ccc(-c2cc(-c3cccc(-c4ccc5ccc6ccc(-c7ccccn7)nc6c5n4)c3)cc(-c3ccccn3)n2)nc1 ; c1ccc(-c2cc(-c3ccccc3-c3ccc4ccc5ccc(-c6cccnc6)nc5c4n3)cc(-c3ccccn3)n2)nc1]

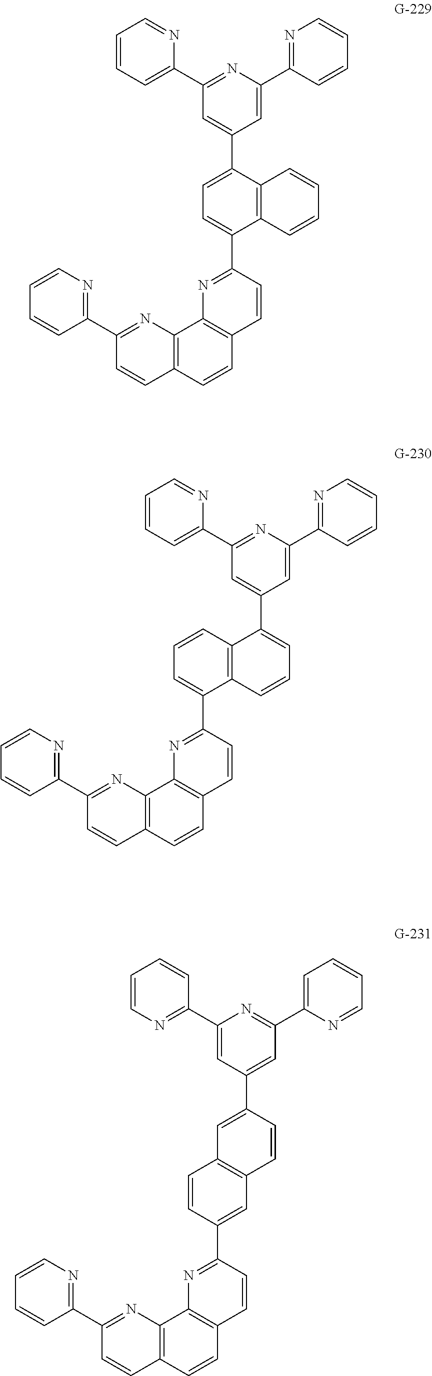

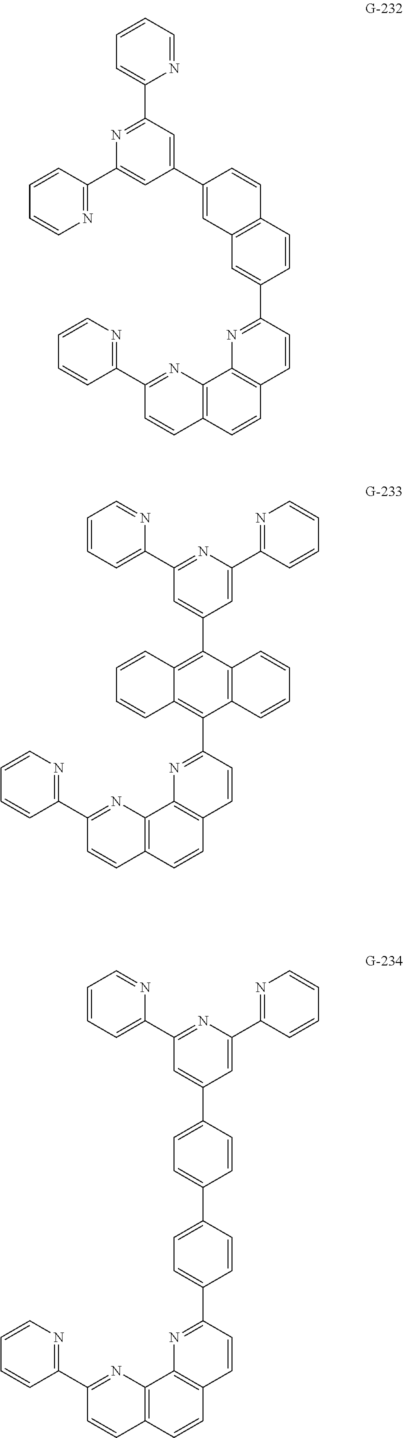 [SMILES: c1ccc(-c2cc(-c3ccc4ccc(-c5ccc6ccc7ccc(-c8ccccn8)nc7c6n5)cc4c3)cc(-c3ccccn3)n2)nc1 ; c1ccc(-c2cc(-c3c4ccccc4c(-c4ccc5ccc6ccc(-c7ccccn7)nc6c5n4)c4ccccc34)cc(-c3ccccn3)n2)nc1 ; c1ccc(-c2cc(-c3ccc(-c4ccc(-c5ccc6ccc7ccc(-c8ccccn8)nc7c6n5)cc4)cc3)cc(-c3ccccn3)n2)nc1]

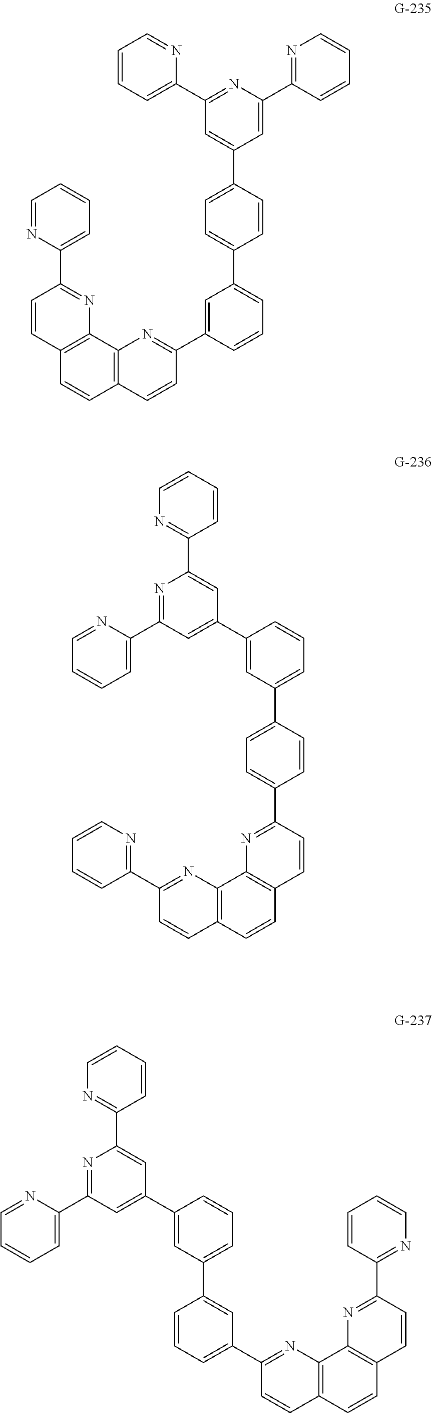 [SMILES: c1ccc(-c2cc(-c3ccc(-c4cccc(-c5ccc6ccc7ccc(-c8ccccn8)nc7c6n5)c4)cc3)cc(-c3ccccn3)n2)nc1 ; c1ccc(-c2cc(-c3cccc(-c4ccc(-c5ccc6ccc7ccc(-c8ccccn8)nc7c6n5)cc4)c3)cc(-c3ccccn3)n2)nc1 ; c1ccc(-c2cc(-c3cccc(-c4cccc(-c5ccc6ccc7ccc(-c8ccccn8)nc7c6n5)c4)c3)cc(-c3ccccn3)n2)nc1]

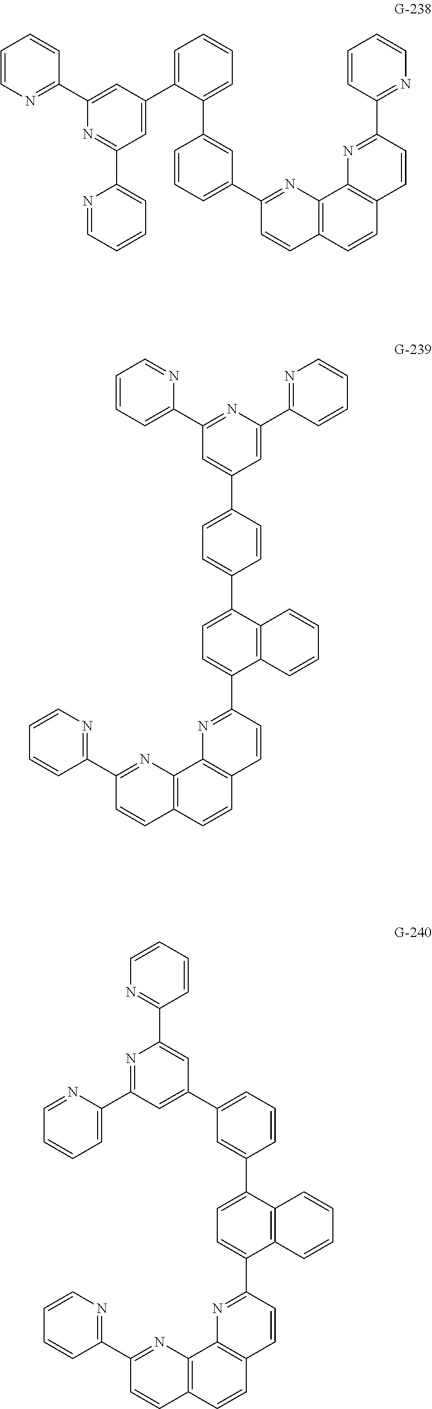 [SMILES: c1ccc(-c2cc(-c3ccccc3-c3cccc(-c4ccc5ccc6ccc(-c7ccccn7)nc6c5n4)c3)cc(-c3ccccn3)n2)nc1 ; c1ccc(-c2cc(-c3ccc(-c4ccc(-c5ccc6ccc7ccc(-c8ccccn8)nc7c6n5)c5ccccc45)cc3)cc(-c3ccccn3)n2)nc1 ; c1ccc(-c2cc(-c3cccc(-c4ccc(-c5ccc6ccc7ccc(-c8ccccn8)nc7c6n5)c5ccccc45)c3)cc(-c3ccccn3)n2)nc1]

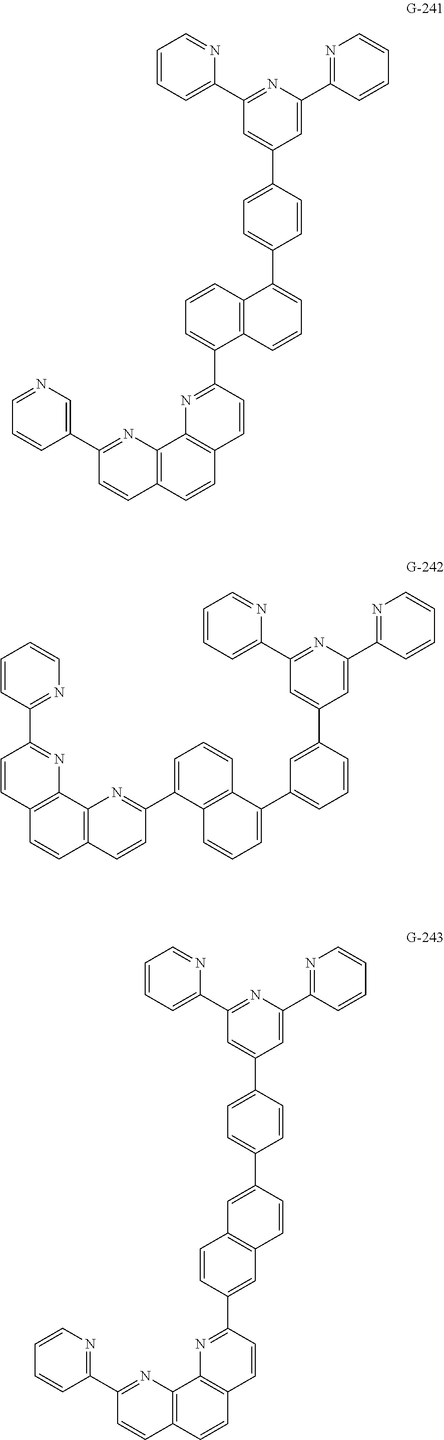

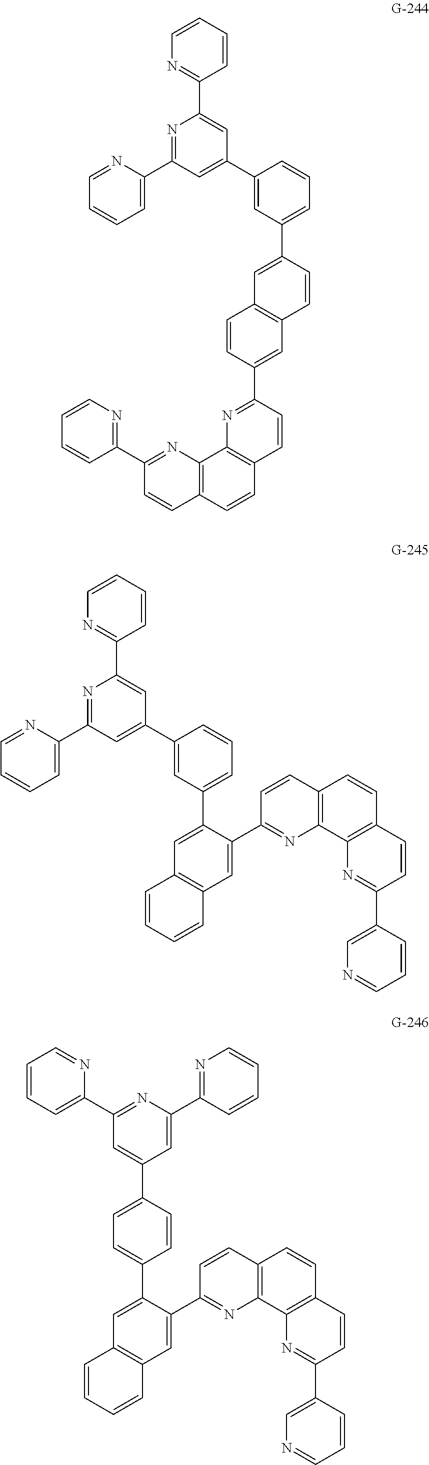 [SMILES: c1ccc(-c2cc(-c3cccc(-c4ccc5cc(-c6ccc7ccc8ccc(-c9ccccn9)nc8c7n6)ccc5c4)c3)cc(-c3ccccn3)n2)nc1 ; c1ccc(-c2cc(-c3cccc(-c4cc5ccccc5cc4-c4ccc5ccc6ccc(-c7cccnc7)nc6c5n4)c3)cc(-c3ccccn3)n2)nc1 ; c1ccc(-c2cc(-c3ccc(-c4cc5ccccc5cc4-c4ccc5ccc6ccc(-c7cccnc7)nc6c5n4)cc3)cc(-c3ccccn3)n2)nc1]

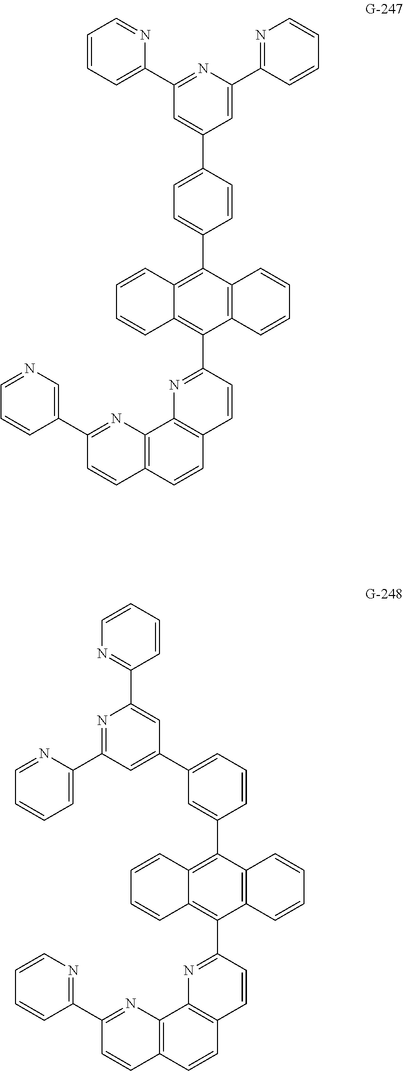 [SMILES: c1ccc(-c2cc(-c3ccc(-c4c5ccccc5c(-c5ccc6ccc7ccc(-c8cccnc8)nc7c6n5)c5ccccc45)cc3)cc(-c3ccccn3)n2)nc1 ; c1ccc(-c2cc(-c3cccc(-c4c5ccccc5c(-c5ccc6ccc7ccc(-c8ccccn8)nc7c6n5)c5ccccc45)c3)cc(-c3ccccn3)n2)nc1]

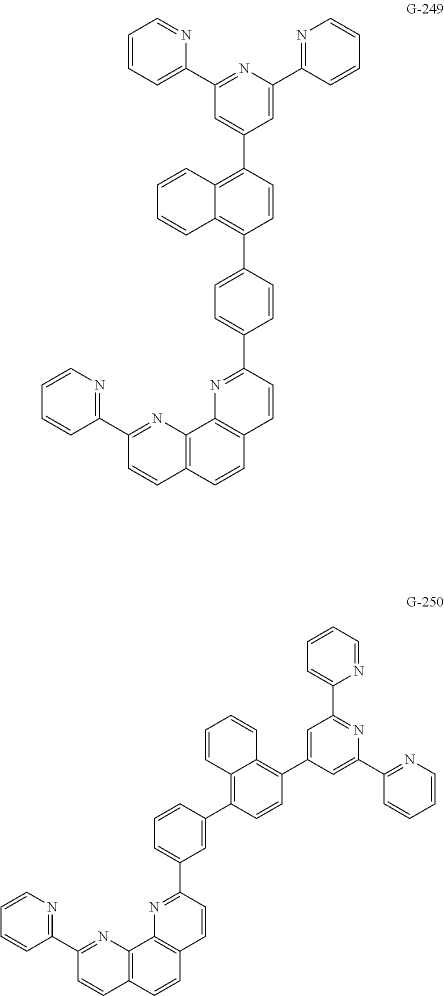 [SMILES: c1ccc(-c2cc(-c3ccc(-c4ccc(-c5ccc6ccc7ccc(-c8ccccn8)nc7c6n5)cc4)c4ccccc34)cc(-c3ccccn3)n2)nc1 ; c1ccc(-c2cc(-c3ccc(-c4cccc(-c5ccc6ccc7ccc(-c8ccccn8)nc7c6n5)c4)c4ccccc34)cc(-c3ccccn3)n2)nc1]

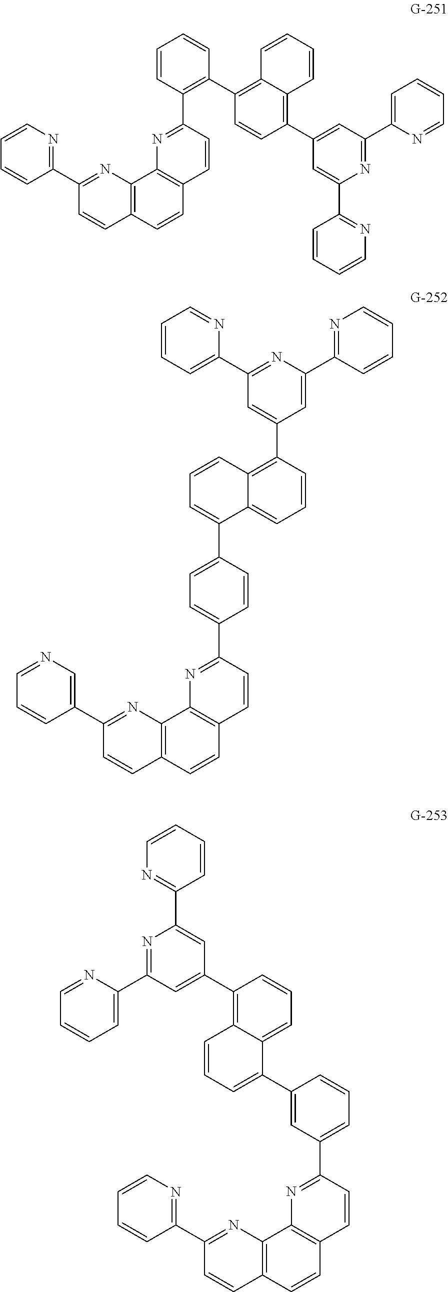

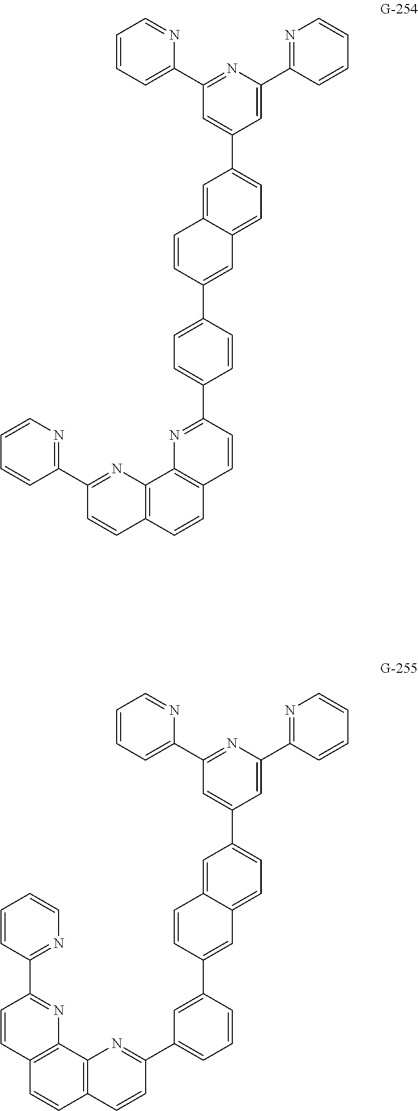 [SMILES: c1ccc(-c2cc(-c3ccc4cc(-c5ccc(-c6ccc7ccc8ccc(-c9ccccn9)nc8c7n6)cc5)ccc4c3)cc(-c3ccccn3)n2)nc1 ; c1ccc(-c2cc(-c3ccc4cc(-c5cccc(-c6ccc7ccc8ccc(-c9ccccn9)nc8c7n6)c5)ccc4c3)cc(-c3ccccn3)n2)nc1]

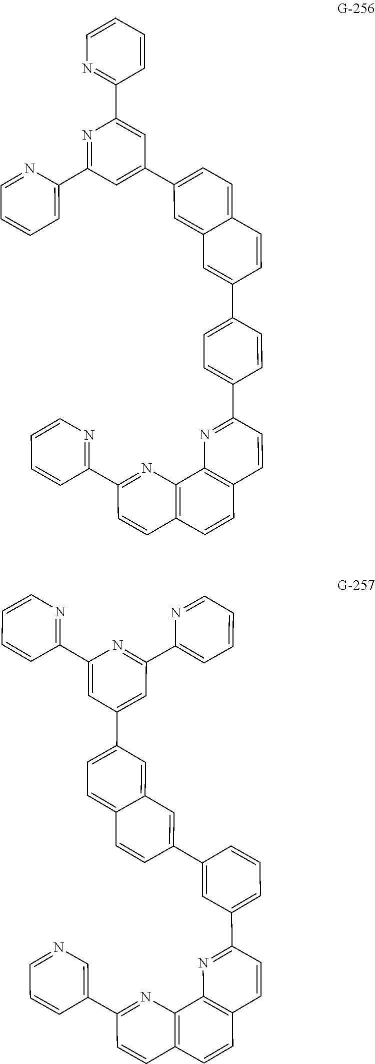 [SMILES: c1ccc(-c2cc(-c3ccc4ccc(-c5ccc(-c6ccc7ccc8ccc(-c9ccccn9)nc8c7n6)cc5)cc4c3)cc(-c3ccccn3)n2)nc1 ; c1ccc(-c2cc(-c3ccc4ccc(-c5cccc(-c6ccc7ccc8ccc(-c9cccnc9)nc8c7n6)c5)cc4c3)cc(-c3ccccn3)n2)nc1]

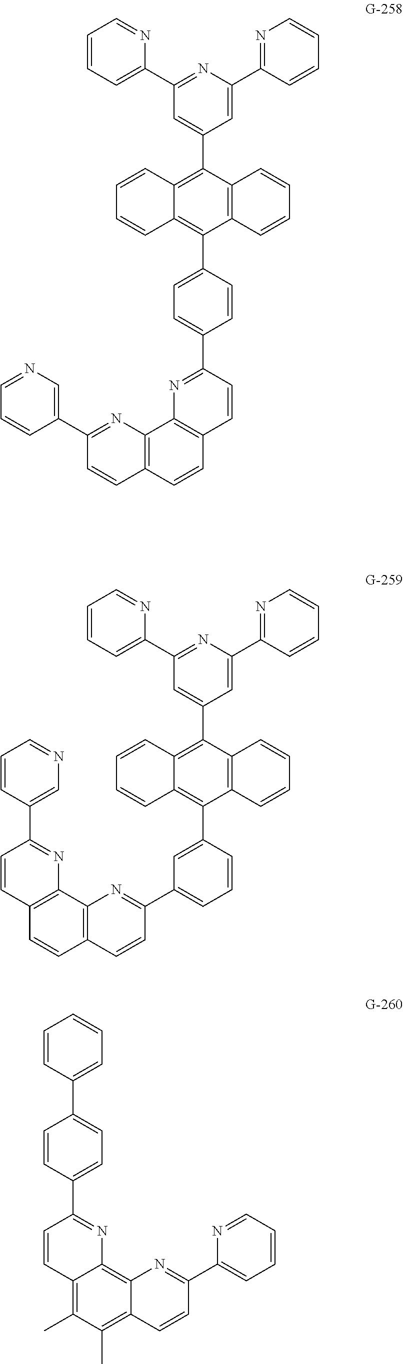 [SMILES: c1ccc(-c2cc(-c3c4ccccc4c(-c4ccc(-c5ccc6ccc7ccc(-c8cccnc8)nc7c6n5)cc4)c4ccccc34)cc(-c3ccccn3)n2)nc1 ; c1ccc(-c2cc(-c3c4ccccc4c(-c4cccc(-c5ccc6ccc7ccc(-c8cccnc8)nc7c6n5)c4)c4ccccc34)cc(-c3ccccn3)n2)nc1 ; Cc1c(C)c2ccc(-c3ccccn3)nc2c2nc(-c3ccc(-c4ccccc4)cc3)ccc12]

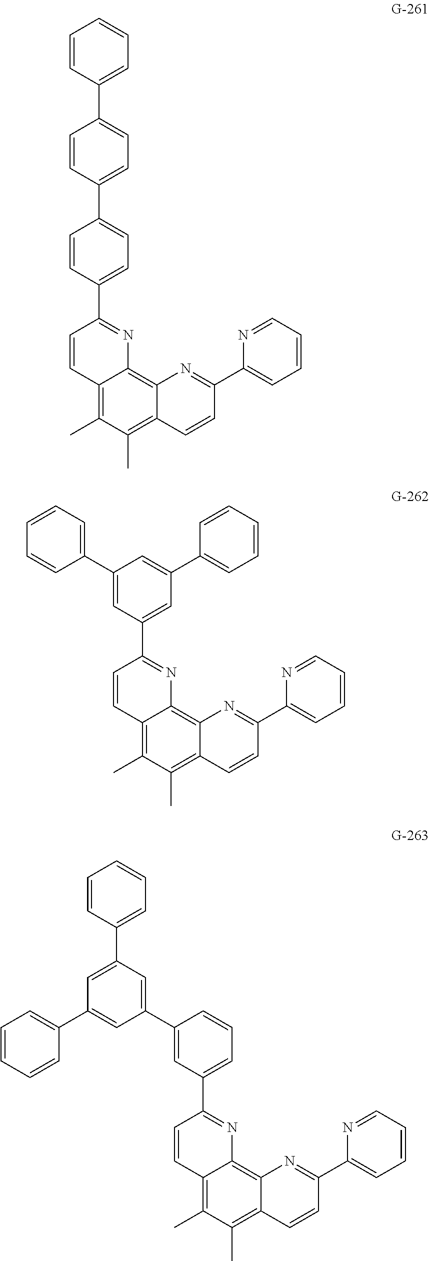 [SMILES: Cc1c(C)c2ccc(-c3ccccn3)nc2c2nc(-c3ccc(-c4ccc(-c5ccccc5)cc4)cc3)ccc12 ; Cc1c(C)c2ccc(-c3ccccn3)nc2c2nc(-c3cc(-c4ccccc4)cc(-c4ccccc4)c3)ccc12 ; Cc1c(C)c2ccc(-c3ccccn3)nc2c2nc(-c3cccc(-c4cc(-c5ccccc5)cc(-c5ccccc5)c4)c3)ccc12]

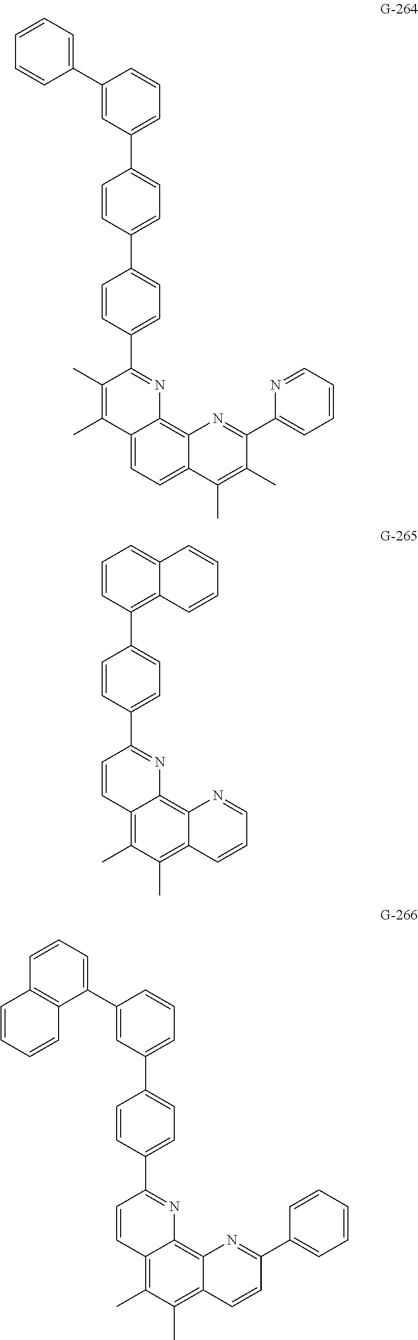

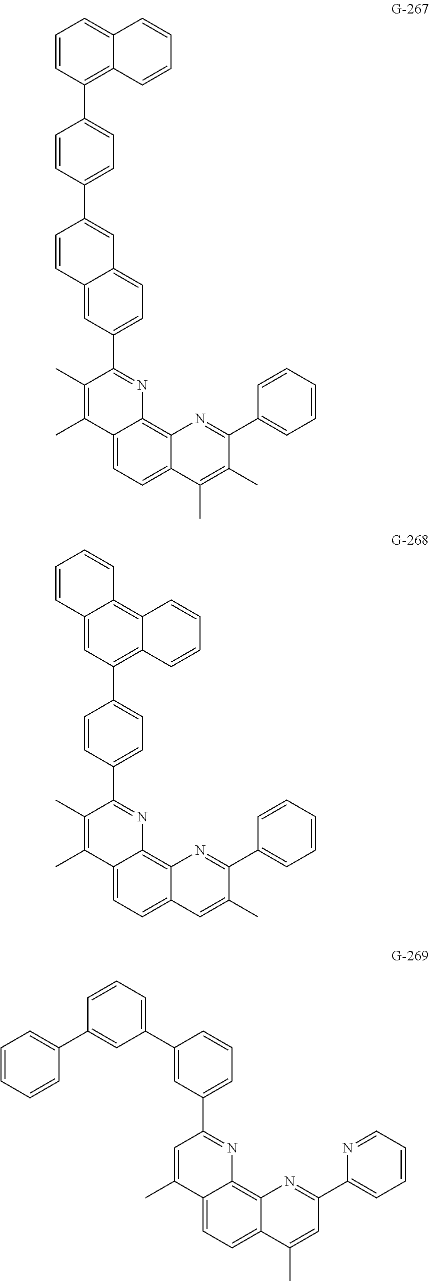 [SMILES: Cc1c(-c2ccccc2)nc2c(ccc3c(C)c(C)c(-c4ccc5cc(-c6ccc(-c7cccc8ccccc78)cc6)ccc5c4)nc32)c1C ; Cc1cc2ccc3c(C)c(C)c(-c4ccc(-c5cc6ccccc6c6ccccc56)cc4)nc3c2nc1-c1ccccc1 ; Cc1cc(-c2cccc(-c3cccc(-c4ccccc4)c3)c2)nc2c1ccc1c(C)cc(-c3ccccn3)nc12]

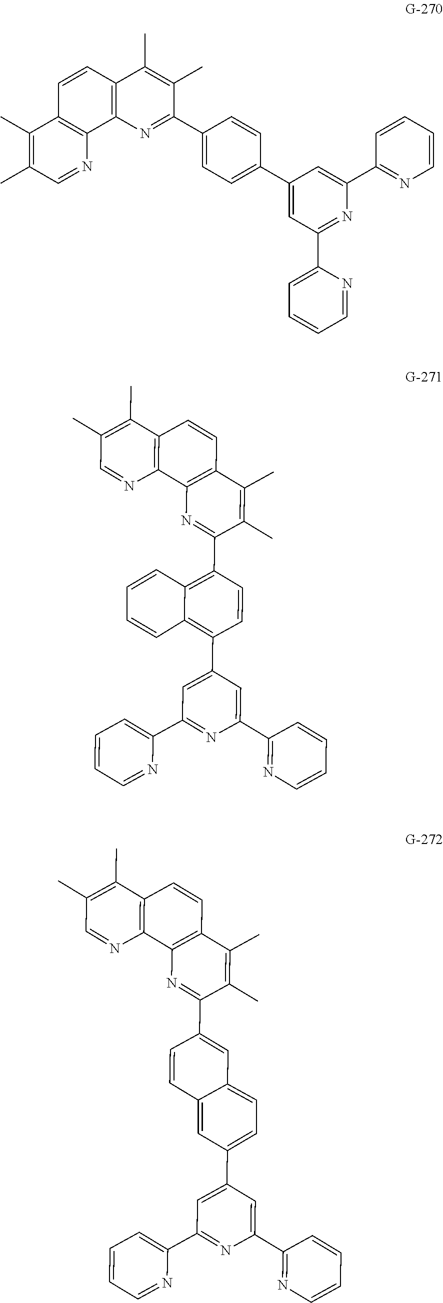 [SMILES: Cc1cnc2c(ccc3c(C)c(C)c(-c4ccc(-c5cc(-c6ccccn6)nc(-c6ccccn6)c5)cc4)nc32)c1C ; Cc1cnc2c(ccc3c(C)c(C)c(-c4ccc(-c5cc(-c6ccccn6)nc(-c6ccccn6)c5)c5ccccc45)nc32)c1C ; Cc1cnc2c(ccc3c(C)c(C)c(-c4ccc5cc(-c6cc(-c7ccccn7)nc(-c7ccccn7)c6)ccc5c4)nc32)c1C]

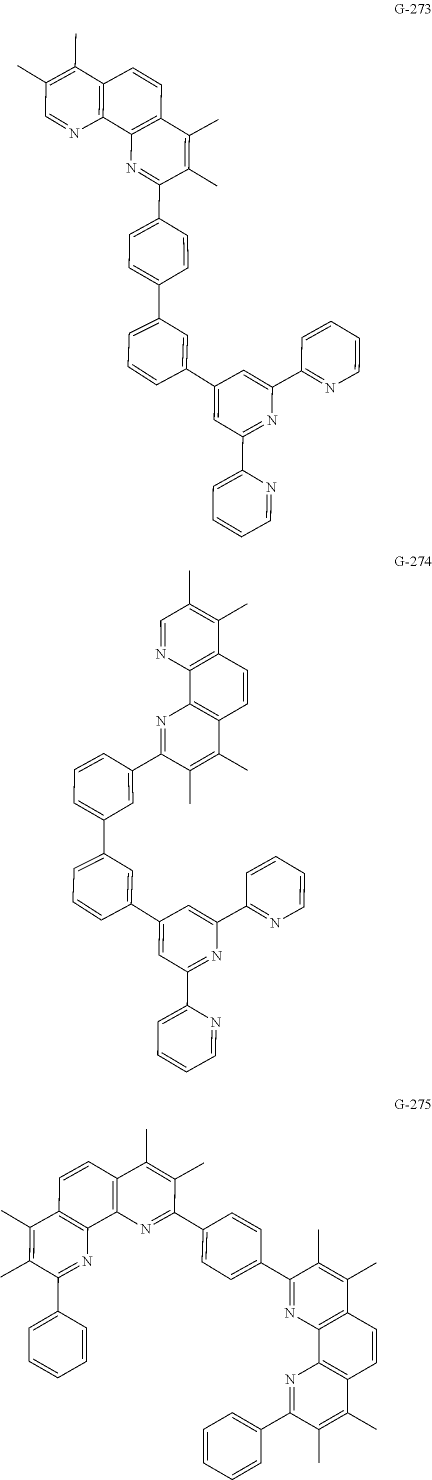

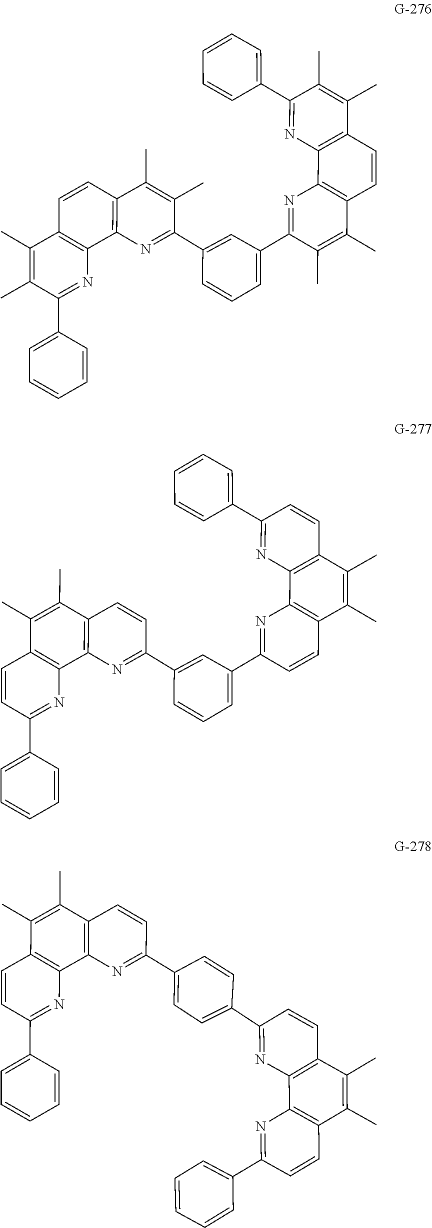

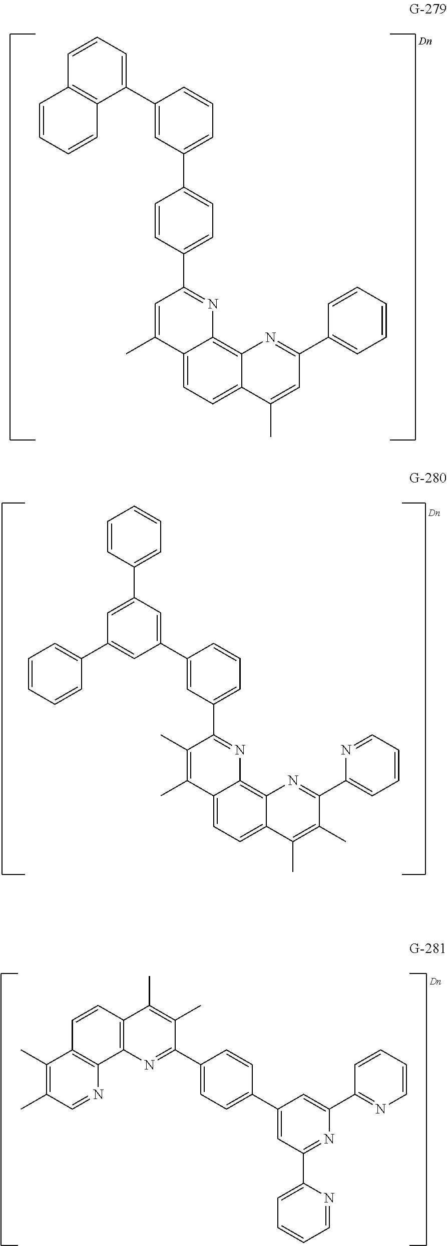

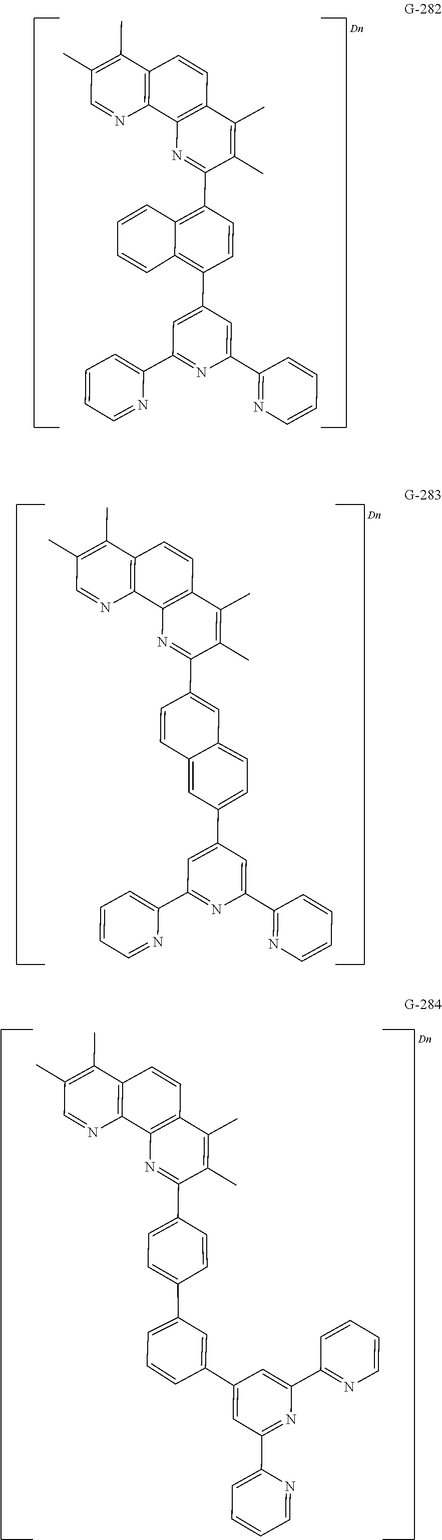

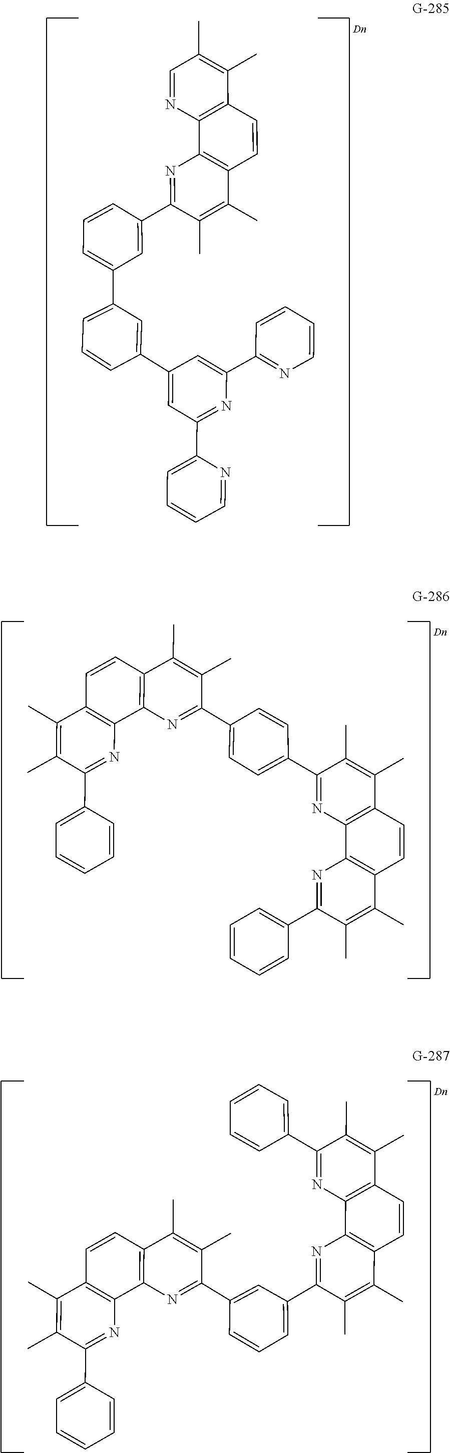

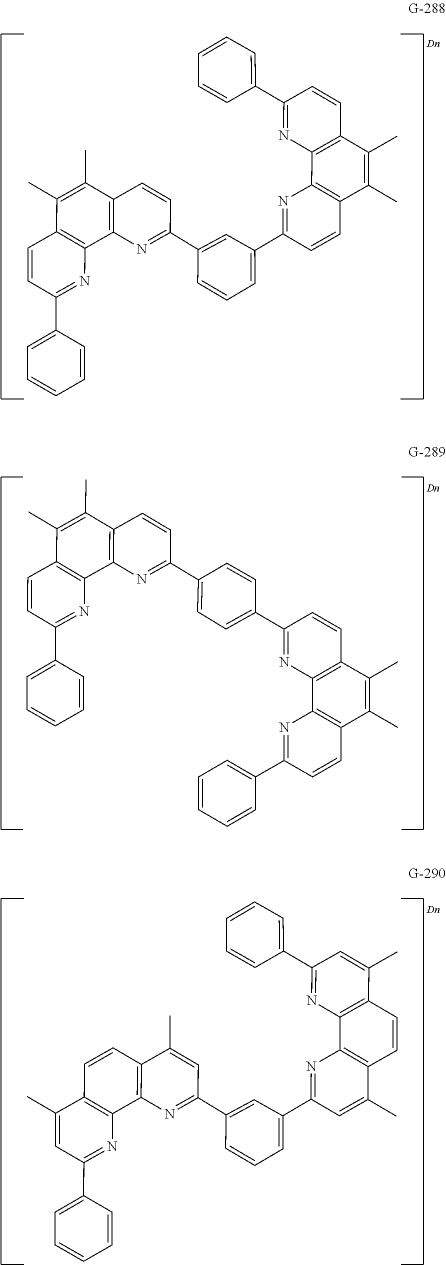

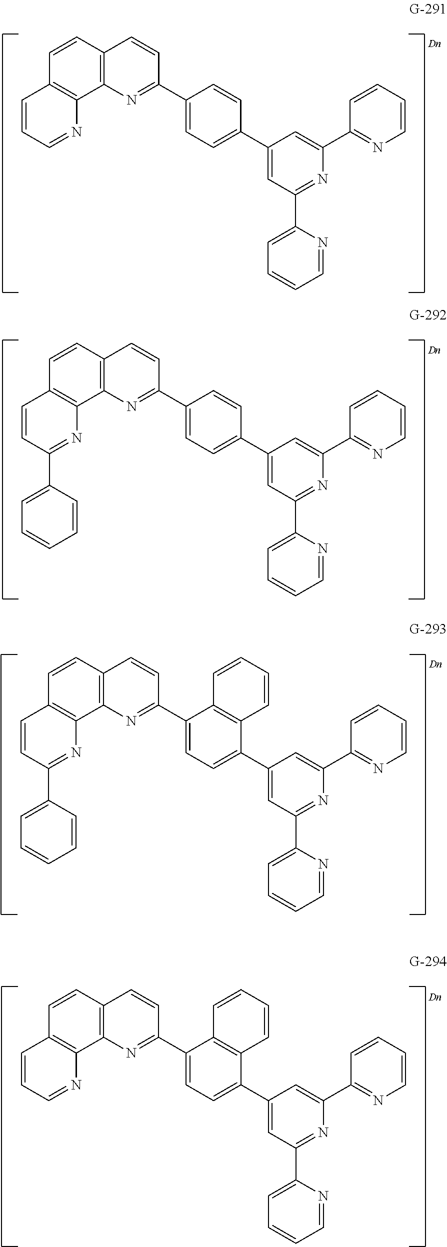

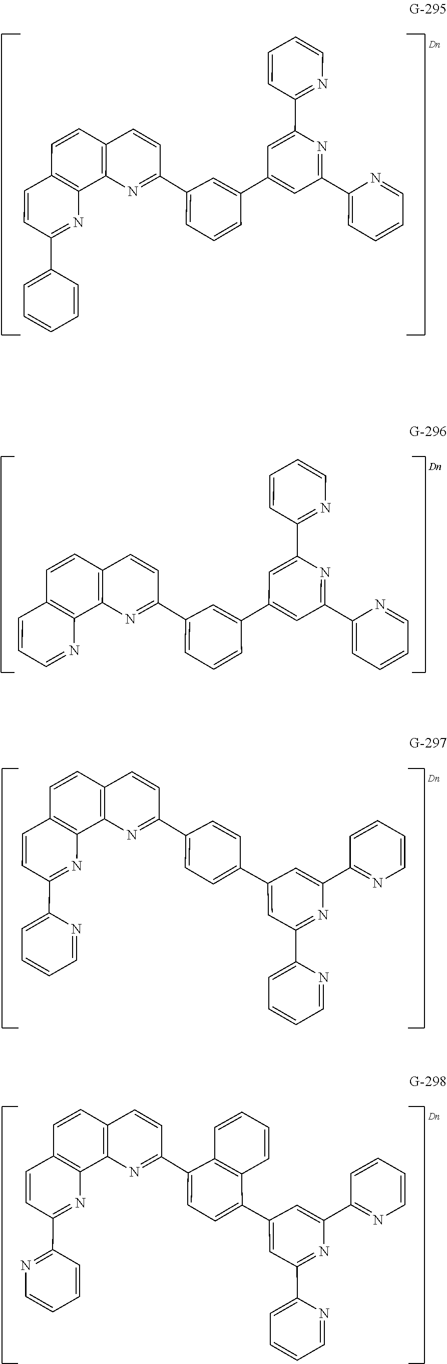

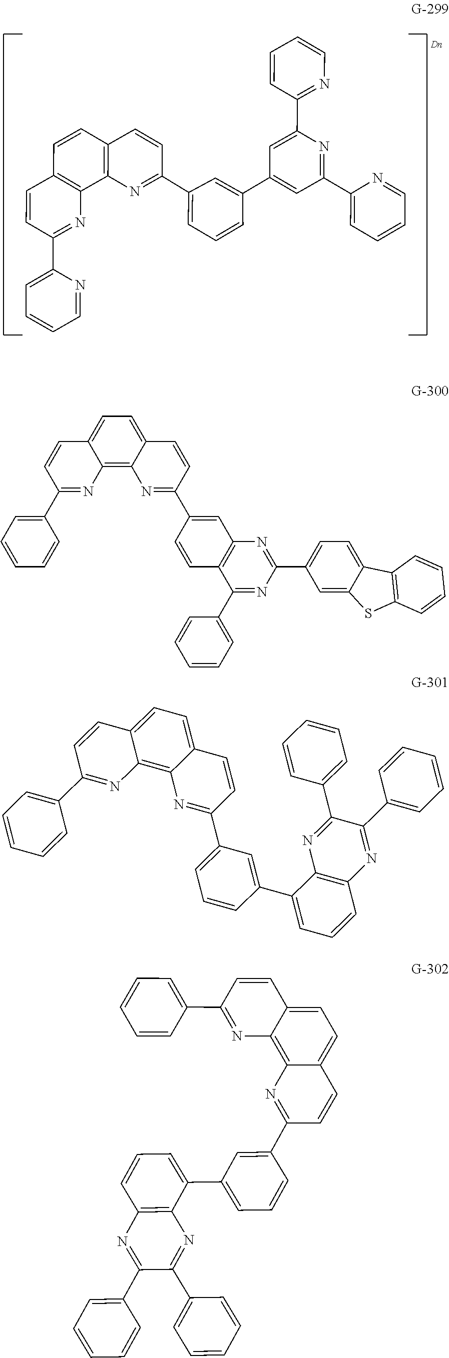

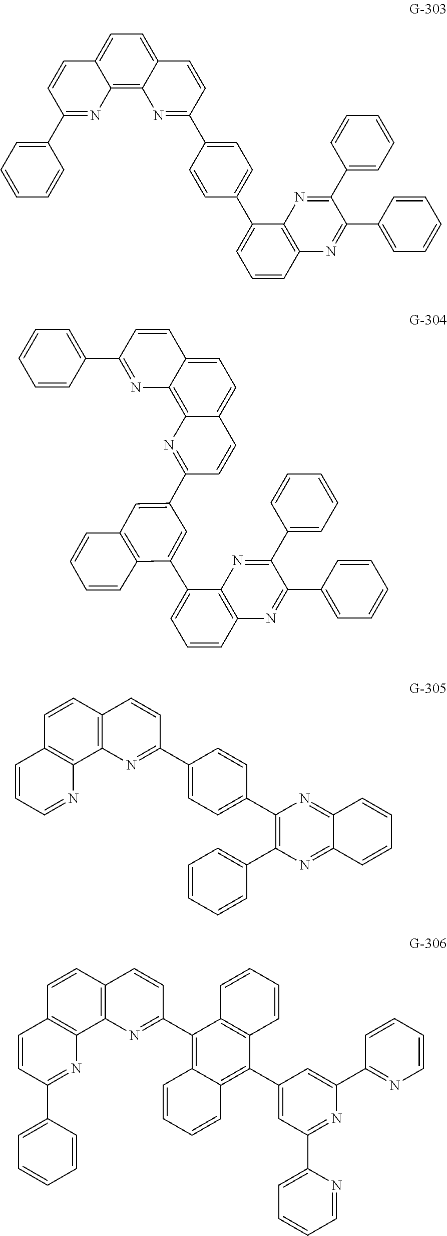 [SMILES: c1ccc(-c2ccc3ccc4ccc(-c5ccc(-c6cccc7nc(-c8ccccc8)c(-c8ccccc8)nc67)cc5)nc4c3n2)cc1 ; c1ccc(-c2ccc3ccc4ccc(-c5cc(-c6cccc7nc(-c8ccccc8)c(-c8ccccc8)nc67)c6ccccc6c5)nc4c3n2)cc1 ; c1ccc(-c2nc3ccccc3nc2-c2ccc(-c3ccc4ccc5cccnc5c4n3)cc2)cc1 ; c1ccc(-c2ccc3ccc4ccc(-c5c6ccccc6c(-c6cc(-c7ccccn7)nc(-c7ccccn7)c6)c6ccccc56)nc4c3n2)cc1]

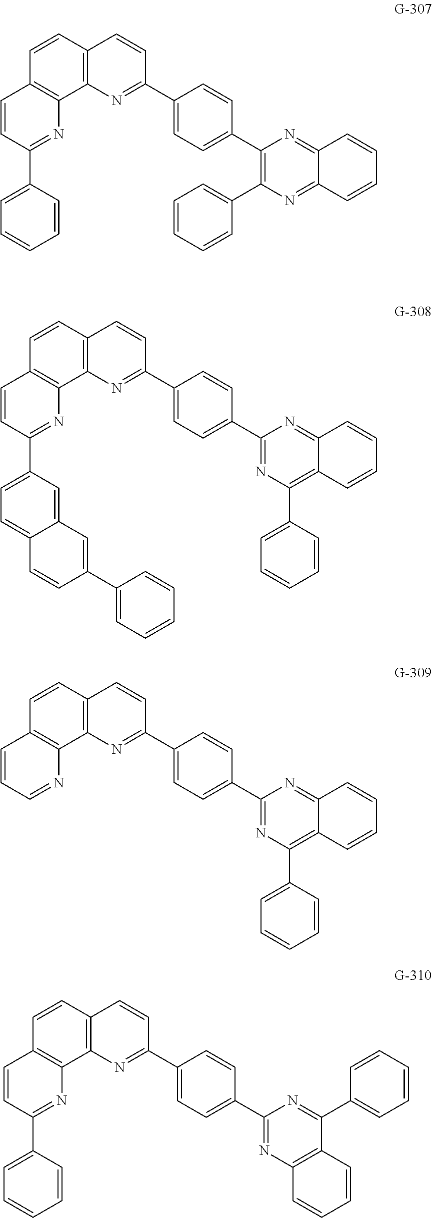 [SMILES: c1ccc(-c2ccc3ccc4ccc(-c5ccc(-c6nc7ccccc7nc6-c6ccccc6)cc5)nc4c3n2)cc1 ; c1ccc(-c2ccc3ccc(-c4ccc5ccc6ccc(-c7ccc(-c8nc(-c9ccccc9)c9ccccc9n8)cc7)nc6c5n4)cc3c2)cc1 ; c1ccc(-c2nc(-c3ccc(-c4ccc5ccc6cccnc6c5n4)cc3)nc3ccccc23)cc1 ; c1ccc(-c2ccc3ccc4ccc(-c5ccc(-c6nc(-c7ccccc7)c7ccccc7n6)cc5)nc4c3n2)cc1]

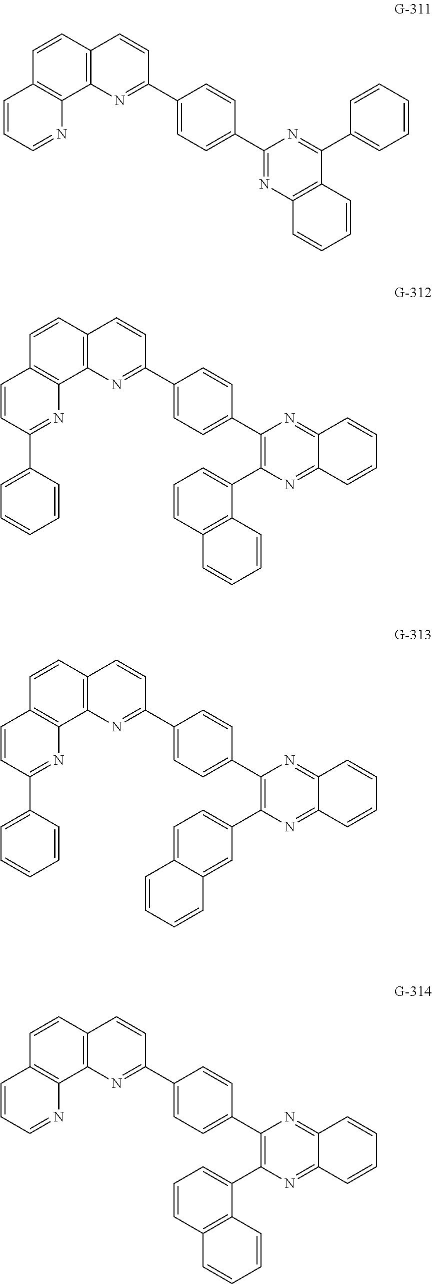 [SMILES: c1ccc(-c2nc(-c3ccc(-c4ccc5ccc6cccnc6c5n4)cc3)nc3ccccc23)cc1 ; c1ccc(-c2ccc3ccc4ccc(-c5ccc(-c6nc7ccccc7nc6-c6cccc7ccccc67)cc5)nc4c3n2)cc1 ; c1ccc(-c2ccc3ccc4ccc(-c5ccc(-c6nc7ccccc7nc6-c6ccc7ccccc7c6)cc5)nc4c3n2)cc1 ; c1ccc2c(-c3nc4ccccc4nc3-c3ccc(-c4ccc5ccc6cccnc6c5n4)cc3)cccc2c1]

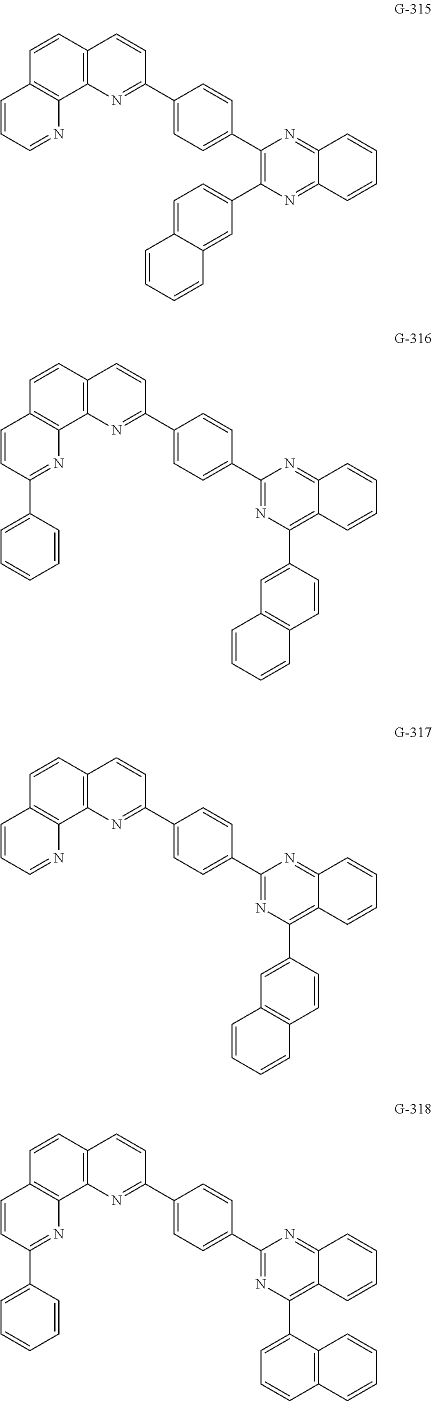 [SMILES: c1ccc2cc(-c3nc4ccccc4nc3-c3ccc(-c4ccc5ccc6cccnc6c5n4)cc3)ccc2c1 ; c1ccc(-c2ccc3ccc4ccc(-c5ccc(-c6nc(-c7ccc8ccccc8c7)c7ccccc7n6)cc5)nc4c3n2)cc1 ; c1ccc2cc(-c3nc(-c4ccc(-c5ccc6ccc7cccnc7c6n5)cc4)nc4ccccc34)ccc2c1 ; c1ccc(-c2ccc3ccc4ccc(-c5ccc(-c6nc(-c7cccc8ccccc78)c7ccccc7n6)cc5)nc4c3n2)cc1]

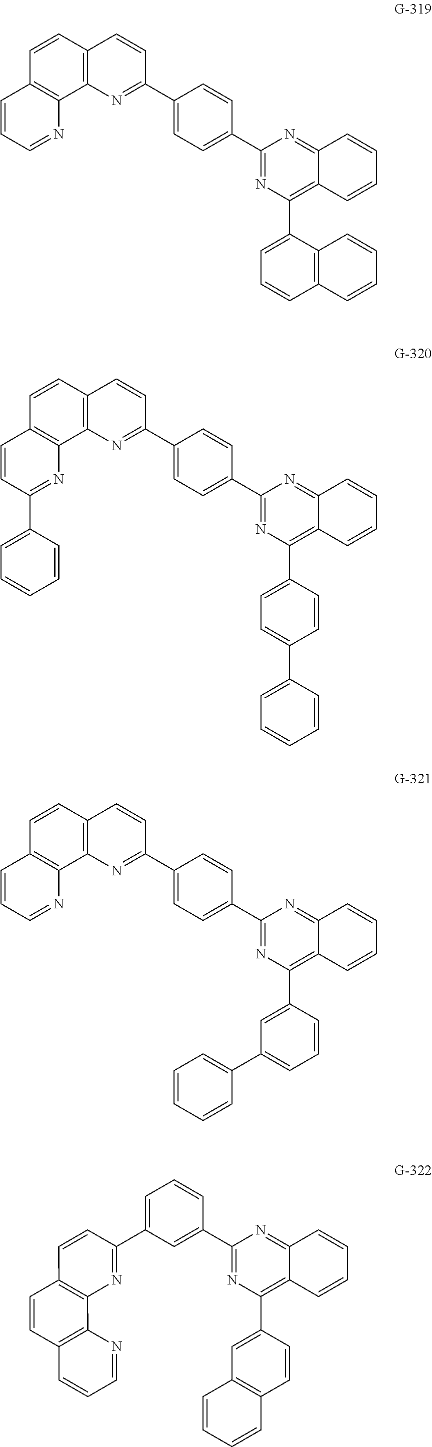 [SMILES: c1ccc2c(-c3nc(-c4ccc(-c5ccc6ccc7cccnc7c6n5)cc4)nc4ccccc34)cccc2c1 ; c1ccc(-c2ccc(-c3nc(-c4ccc(-c5ccc6ccc7ccc(-c8ccccc8)nc7c6n5)cc4)nc4ccccc34)cc2)cc1 ; c1ccc(-c2cccc(-c3nc(-c4ccc(-c5ccc6ccc7cccnc7c6n5)cc4)nc4ccccc34)c2)cc1 ; c1cc(-c2ccc3ccc4cccnc4c3n2)cc(-c2nc(-c3ccc4ccccc4c3)c3ccccc3n2)c1]

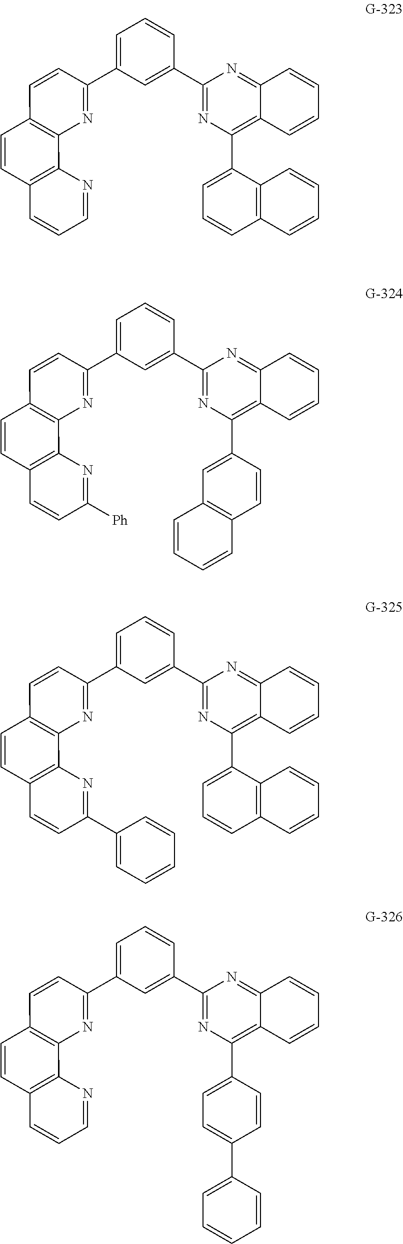

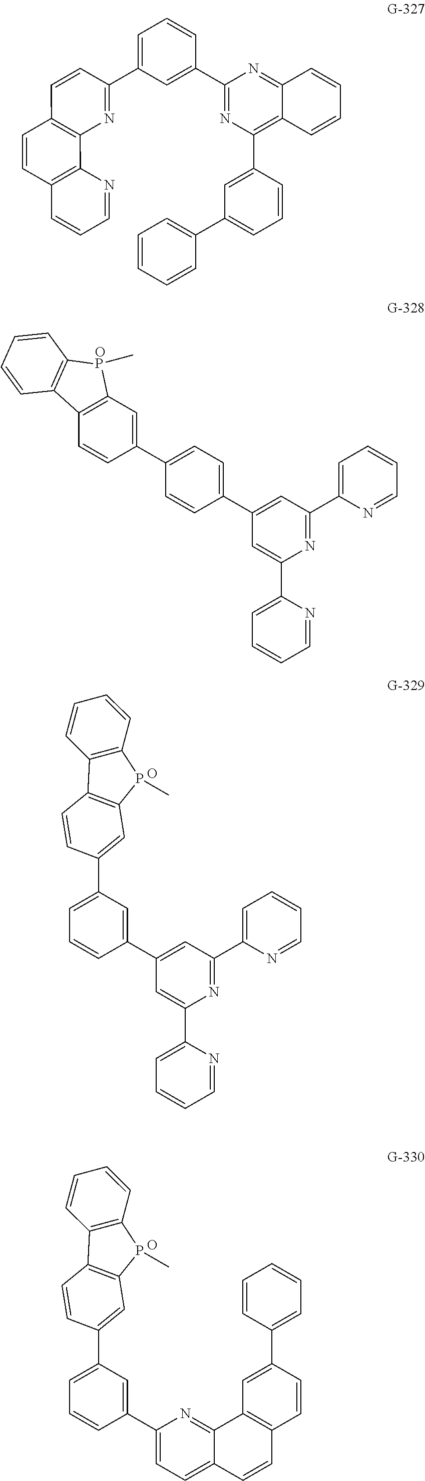

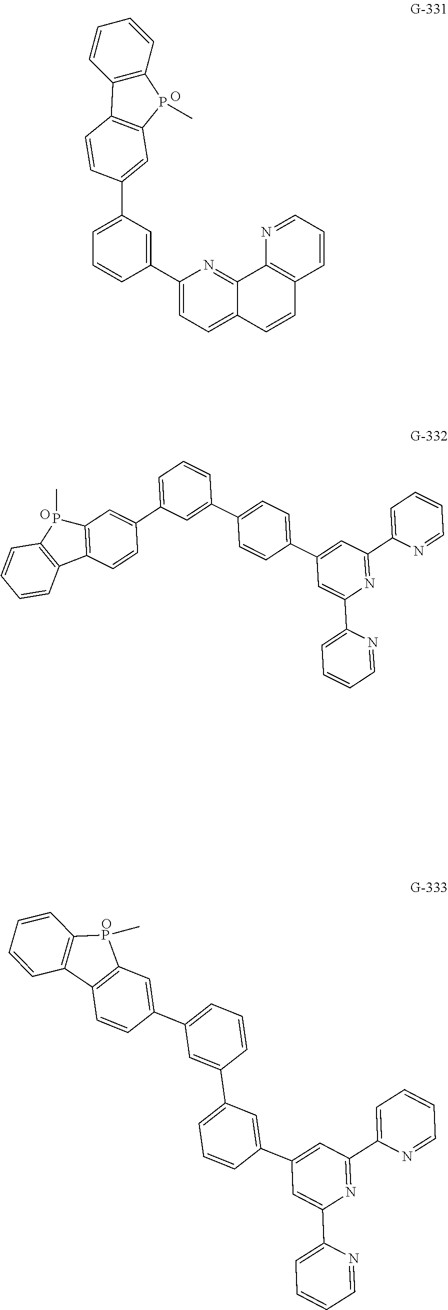

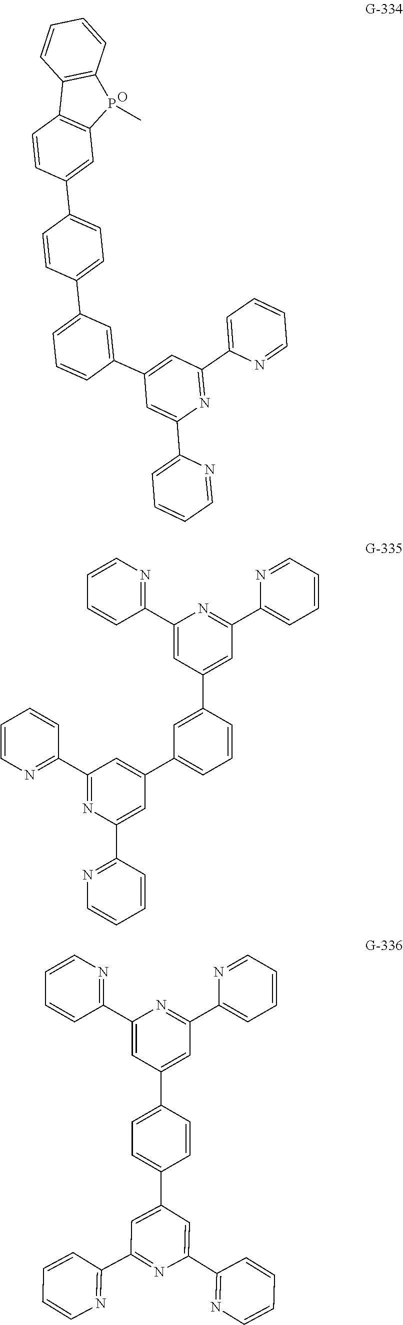

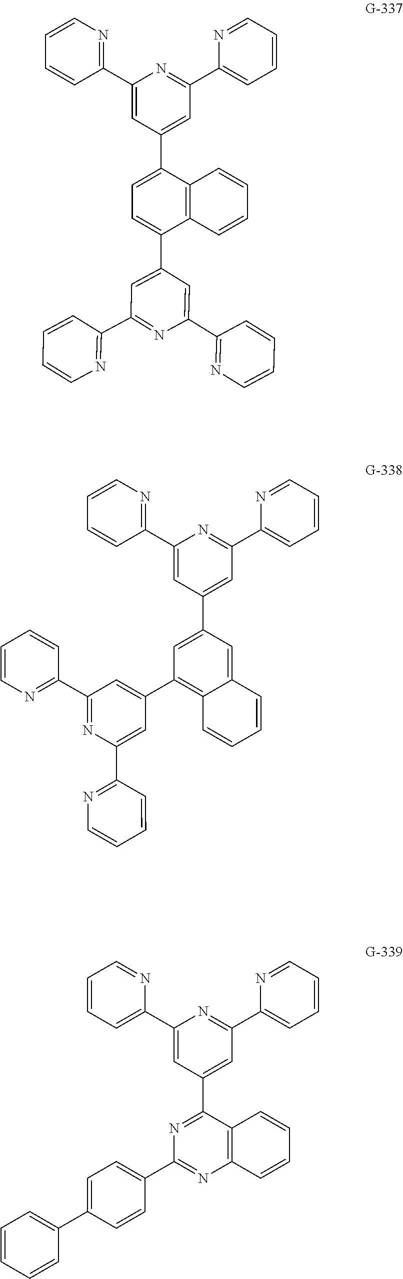 [SMILES: c1ccc(-c2cc(-c3ccc(-c4cc(-c5ccccn5)nc(-c5ccccn5)c4)c4ccccc34)cc(-c3ccccn3)n2)nc1 ; c1ccc(-c2cc(-c3cc(-c4cc(-c5ccccn5)nc(-c5ccccn5)c4)c4ccccc4c3)cc(-c3ccccn3)n2)nc1 ; c1ccc(-c2ccc(-c3nc(-c4cc(-c5ccccn5)nc(-c5ccccn5)c4)c4ccccc4n3)cc2)cc1]

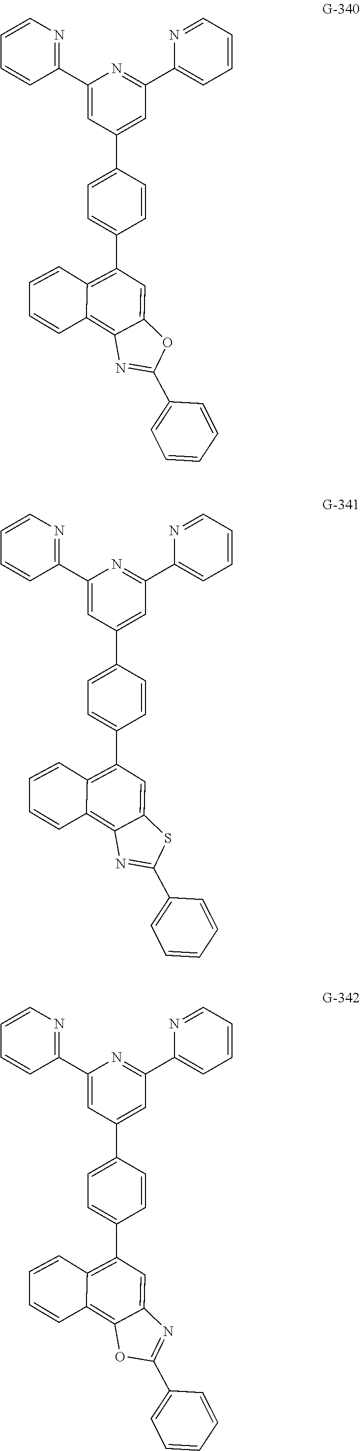 [SMILES: c1ccc(-c2nc3c(cc(-c4ccc(-c5cc(-c6ccccn6)nc(-c6ccccn6)c5)cc4)c4ccccc43)o2)cc1 ; c1ccc(-c2nc3c(cc(-c4ccc(-c5cc(-c6ccccn6)nc(-c6ccccn6)c5)cc4)c4ccccc43)s2)cc1 ; c1ccc(-c2nc3cc(-c4ccc(-c5cc(-c6ccccn6)nc(-c6ccccn6)c5)cc4)c4ccccc4c3o2)cc1]

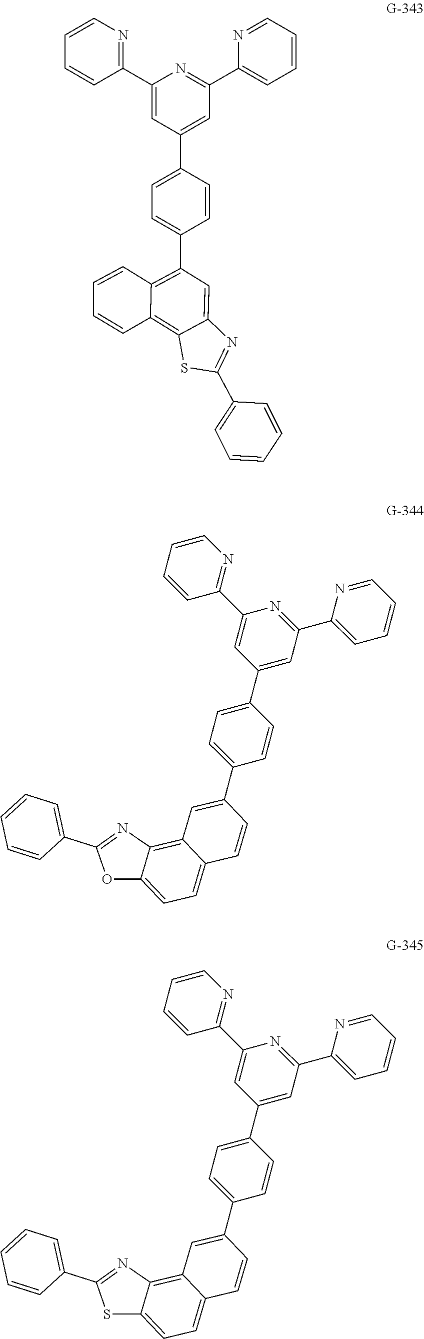 [SMILES: c1ccc(-c2nc3cc(-c4ccc(-c5cc(-c6ccccn6)nc(-c6ccccn6)c5)cc4)c4ccccc4c3s2)cc1 ; c1ccc(-c2nc3c(ccc4ccc(-c5ccc(-c6cc(-c7ccccn7)nc(-c7ccccn7)c6)cc5)cc43)o2)cc1 ; c1ccc(-c2nc3c(ccc4ccc(-c5ccc(-c6cc(-c7ccccn7)nc(-c7ccccn7)c6)cc5)cc43)s2)cc1]

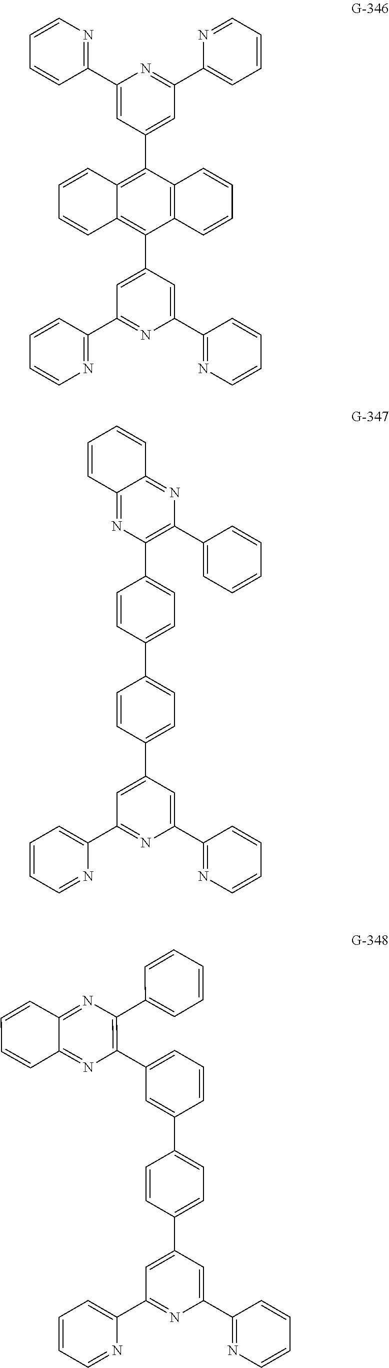 [SMILES: c1ccc(-c2cc(-c3c4ccccc4c(-c4cc(-c5ccccn5)nc(-c5ccccn5)c4)c4ccccc34)cc(-c3ccccn3)n2)nc1 ; c1ccc(-c2nc3ccccc3nc2-c2ccc(-c3ccc(-c4cc(-c5ccccn5)nc(-c5ccccn5)c4)cc3)cc2)cc1 ; c1ccc(-c2nc3ccccc3nc2-c2cccc(-c3ccc(-c4cc(-c5ccccn5)nc(-c5ccccn5)c4)cc3)c2)cc1]

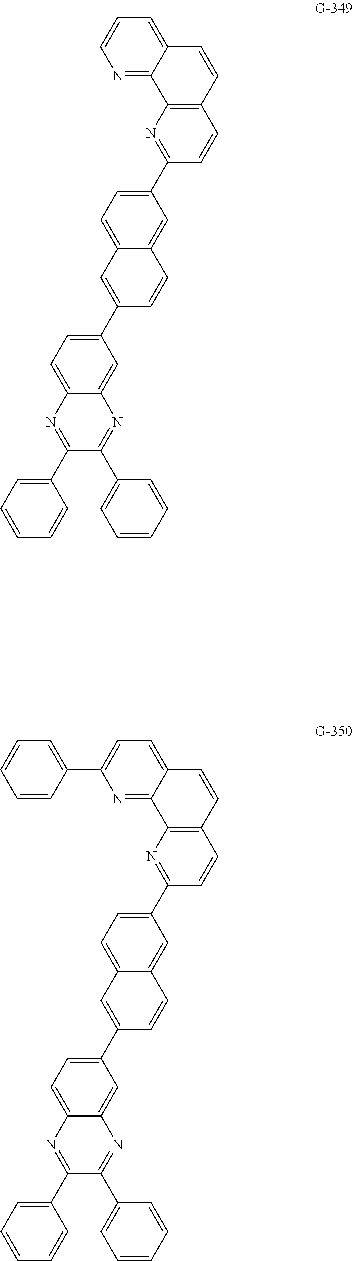 [SMILES: c1ccc(-c2nc3ccc(-c4ccc5cc(-c6ccc7ccc8cccnc8c7n6)ccc5c4)cc3nc2-c2ccccc2)cc1 ; c1ccc(-c2ccc3ccc4ccc(-c5ccc6cc(-c7ccc8nc(-c9ccccc9)c(-c9ccccc9)nc8c7)ccc6c5)nc4c3n2)cc1]

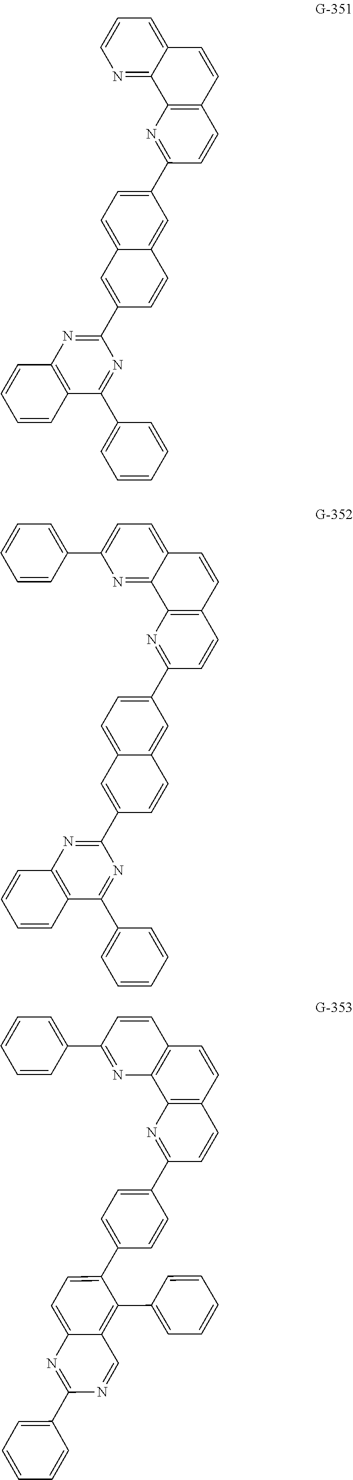

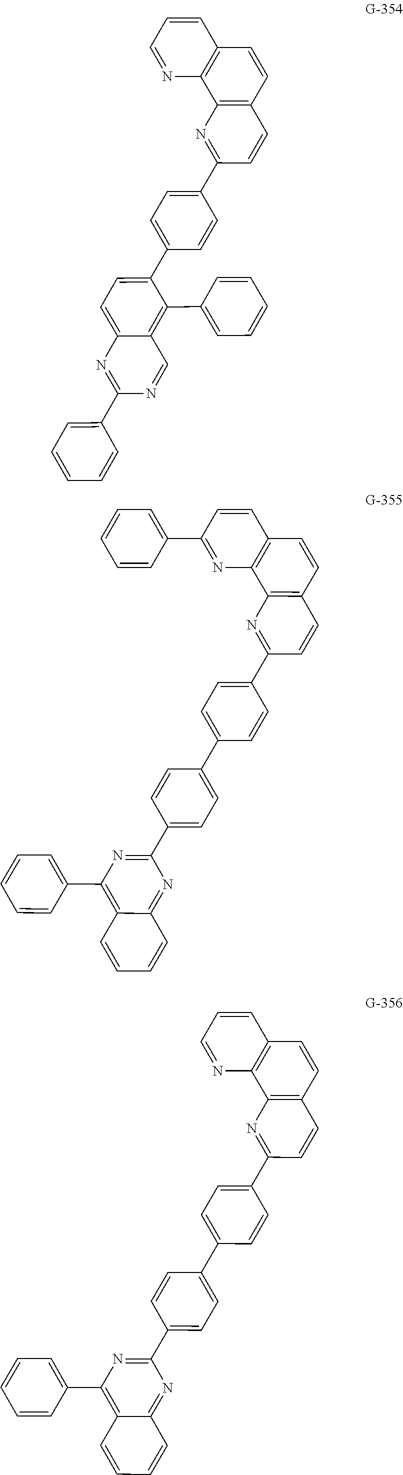

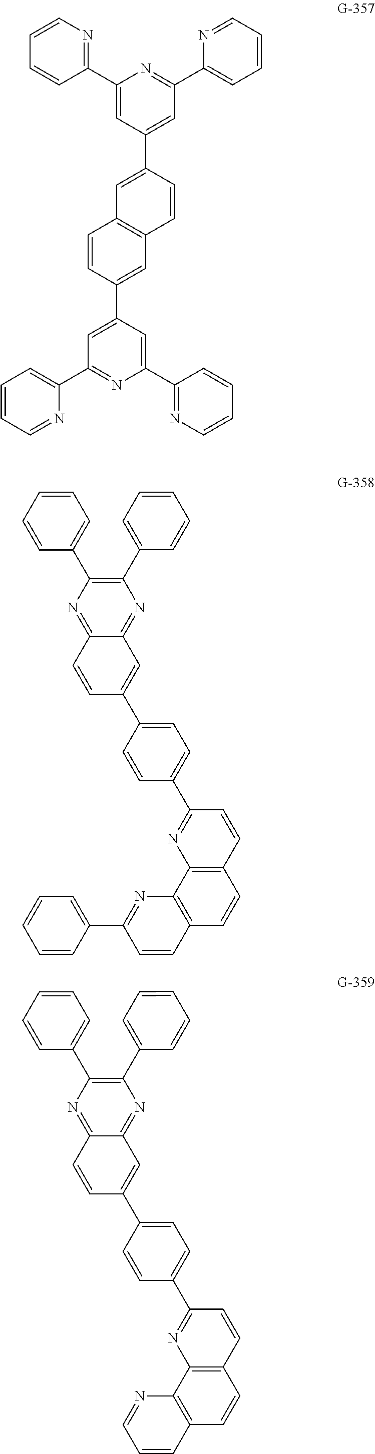

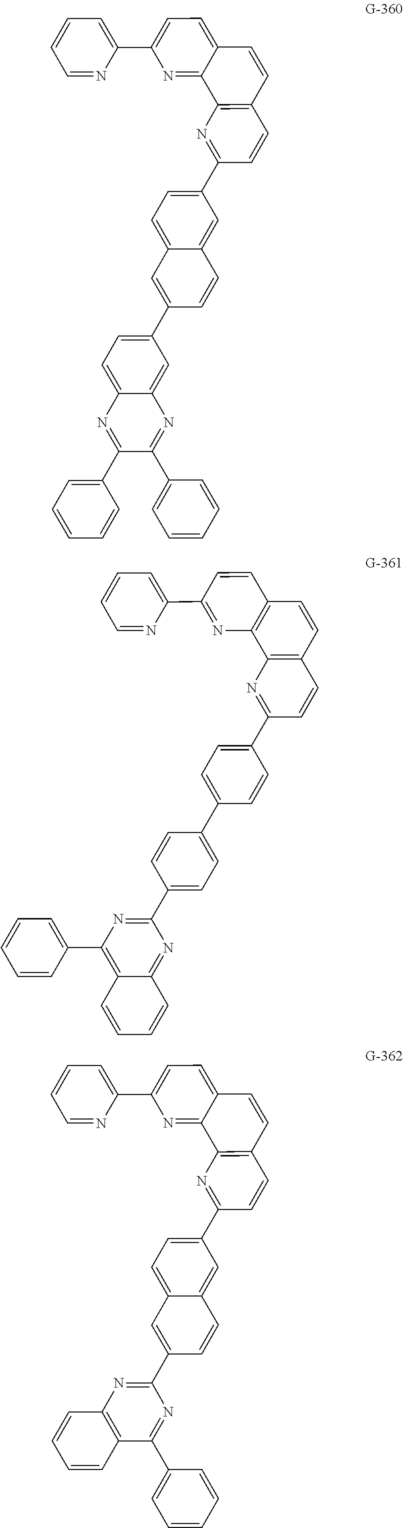

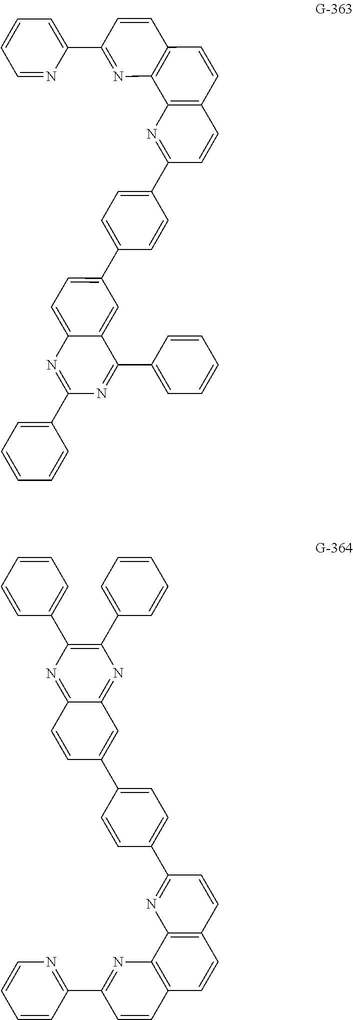 [SMILES: c1ccc(-c2nc(-c3ccccc3)c3cc(-c4ccc(-c5ccc6ccc7ccc(-c8ccccn8)nc7c6n5)cc4)ccc3n2)cc1 ; c1ccc(-c2nc3ccc(-c4ccc(-c5ccc6ccc7ccc(-c8ccccn8)nc7c6n5)cc4)cc3nc2-c2ccccc2)cc1]

In the compounds, Dₙ indicates that n hydrogen atoms are replaced with deuterium atoms, wherein n is an integer ranging from 1 to the maximum number of hydrogen atoms present in the compound.

According to another embodiment of the present disclosure, an organic electroluminescent device is provided, comprising: a first electrode; a second electrode opposing the first electrode; and two or more light-emitting units disposed between the first electrode and the second electrode, each including at least one light-emitting layer, wherein at least one of the light-emitting units sequentially comprises a first light-emitting layer, a second light-emitting layer, and a third light-emitting layer that are sequentially adjacent to one another. The first light-emitting layer is disposed adjacent to the first electrode, the third light-emitting layer is disposed adjacent to the second electrode, and the first and third light-emitting layers include the same compound.

According to another embodiment of the present disclosure, an organic electroluminescent device is provided, comprising: a first electrode; a second electrode opposing the first electrode; and a first light-emitting layer, a second light-emitting layer, and a third light-emitting layer sequentially disposed between the first electrode and the second electrode, which are adjacent to one another. The first light-emitting layer is disposed adjacent to the first electrode, the third light-emitting layer is disposed adjacent to the second electrode, and the host materials of the adjacent light-emitting layers are mutually different.

According to one embodiment, the host materials of each of the first light-emitting layer, the second light-emitting layer, and the third light-emitting layer may include different compounds.

According to another embodiment, the host materials of each of the first light-emitting layer and the third light-emitting layer may include the same compound.

In one embodiment, the host material of at least two layers among each of the first light-emitting layer, the second light-emitting layer, and the third light-emitting layer may include a composition including two or more compounds, wherein the composition of each layer may comprise the same compound but the ratio of the compounds in each composition may differ.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. The organic electroluminescent device may include a plurality of light-emitting units, wherein each of the light-emitting units may include a hole transport band, a light-emitting layer, and an electron transport band, and the hole transport band may include a hole injection layer and a hole transport layer, and the electron transport zone may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. At this time, the plurality of charge generation layers may be the same as or different from one another. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of an anode and a cathode without a separate internal electrode located between the stacks.

The charge generation layer may be composed of an n-type charge generation layer and a p-type charge generation layer, and the n-type charge generation layer may be doped with an alkali metal, an alkaline earth metal, or a compound of an alkali metal and an alkaline earth metal. The alkali metal may comprise one selected from the group consisting of Li, Na, K, Rb, Cs, Fr, Yb, and combinations thereof, and the alkaline earth metal may comprise one selected from the group consisting of Be, Mg, Ca, Sr, Ba, Ra, and combinations thereof. The p-type charge generation layer may be made of a metal or an organic material doped with a p-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the p-type dopant and host materials used in the p-type doped organic material.

According to one embodiment, the light-emitting layers may further include one or more dopants. As the dopant in the organic electroluminescent device of the present disclosure, one or more phosphorescent or fluorescent dopants may be used, with fluorescent dopants being preferred. The fluorescent dopant material applied to the organic electroluminescent device of the present disclosure may use a compound represented by the following Formula D, but is not limited thereto.

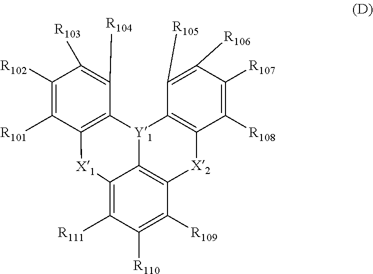

In Formula D,
R₁₀₁ to R₁₁₁ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N-(Ar′₄)(Ar′₅), or may be linked to adjacent substituents to form a ring;
Y′₁ represents B;
X′₁ and X′₂ each independently represent NR′;
R′ represents hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N-(Ar′₄)(Ar′₅); or may be linked to at least one of R₁₀₁, R₁₀₈, R₁₀₉, and R₁₁₁ to form a ring;
L′₄ represents a single bond, a substituted or unsubstituted (C₆-C₃₀)arylene, or a substituted or unsubstituted (3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring of a (C₃-C₃₀) aliphatic ring and a (C₆-C₃₀) aromatic ring, a substituted or unsubstituted (C₆-C₃₀)aryl, or a substituted or unsubstituted (3- to 30-membered)heteroaryl.

Preferably, R₁₀₁ to R₁₁₁ each independently may be hydrogen, deuterium, a substituted or unsubstituted (C₁-C₂₀)alkyl, a substituted or unsubstituted (C₆-C₂₅)aryl, a substituted or unsubstituted (5- to 20-membered)heteroaryl, or -L′₄-N-(Ar′₄)(Ar′₅), or may be linked to adjacent substituents to form a ring.

More preferably, R₁₀₁ to R₁₁₁ each independently may be hydrogen, deuterium, unsubstituted (C₁-C₁₀)alkyl; (C₆-C₁₈)aryl unsubstituted or substituted with at least one of (C₁-C₁₀)alkyl, (13- to 18-membered)heteroaryl, and di(C₆-C₁₈)arylamino; (5- to 18-membered)heteroaryl unsubstituted or substituted with at least one (C₁-C₁₀)alkyl; or -L′₄-N-(Ar′₄)(Ar′₅), or may be linked to adjacent substituents to form a ring. For example, R₁₀₁ to R₁₁₁ each independently may be hydrogen, methyl, tert-butyl, a substituted or unsubstituted phenyl, biphenyl, terphenyl, triphenylenyl, carbazolyl, phenoxazinyl, phenothiazinyl, dimethylacridinyl, dimethylxanthenyl, diphenylamino unsubstituted or substituted with at least one of methyl and diphenylamino, phenylnaphthylamino, dibiphenylamino, phenylamino substituted with phenylcarbazolyl or dibenzofuranyl, (17- to 21-membered)heteroaryl substituted with at least one of methyl and phenyl, or may be linked to adjacent substituents to form a benzene ring, an indole ring substituted with at least one of phenyl and diphenylamino, a benzofuran ring, a benzothiophene ring, or a 19-membered hetero ring substituted with at least one methyl. The substituent of the substituted phenyl may be at least one of methyl, carbazolyl, dibenzofuranyl, diphenylamino, phenoxazinyl, phenothiazinyl, and dimethylacridinyl.

According to one embodiment, the following compounds may be specifically exemplified, but are not limited thereto.

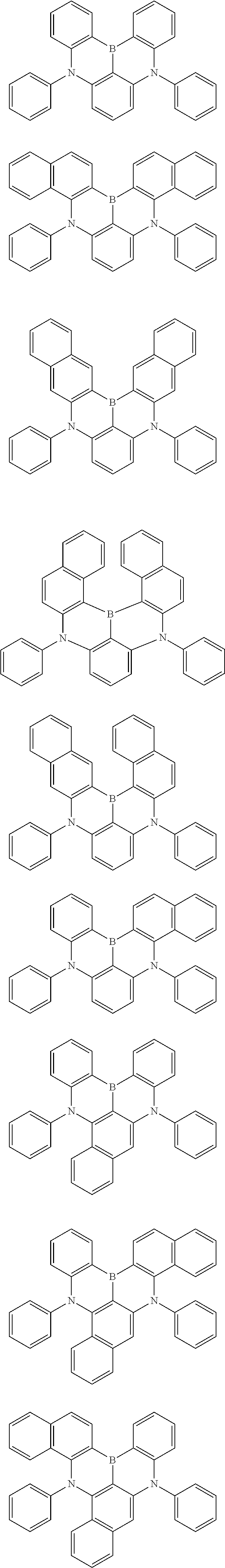

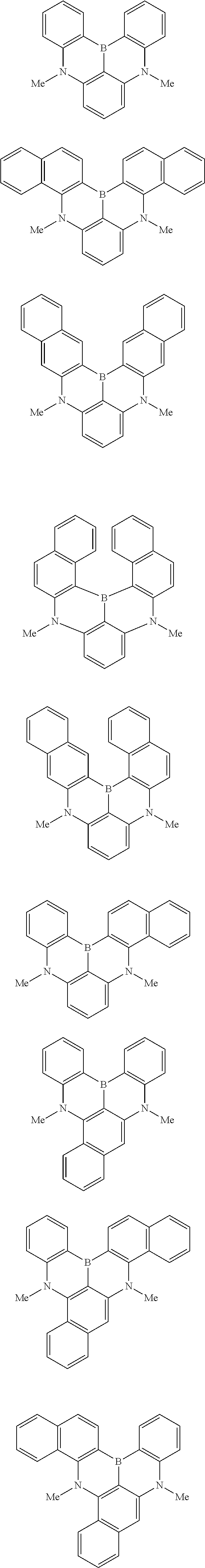

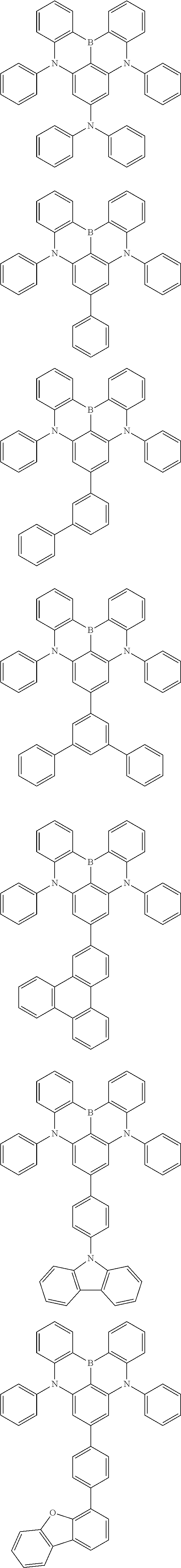

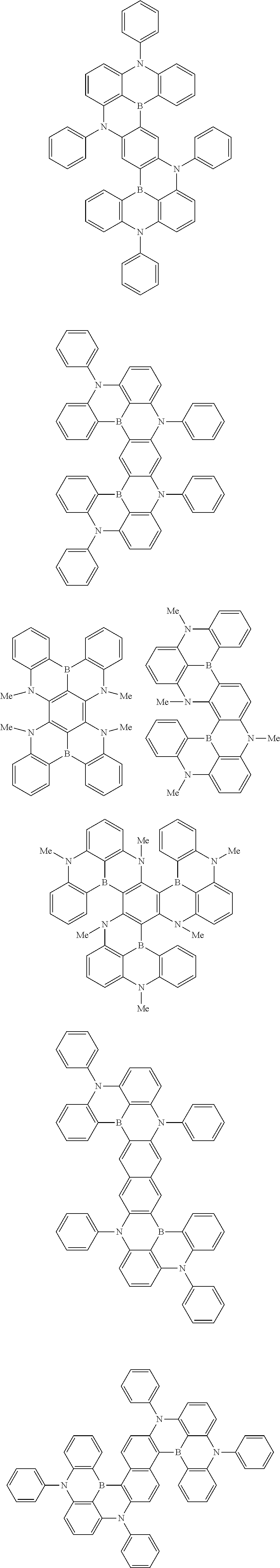

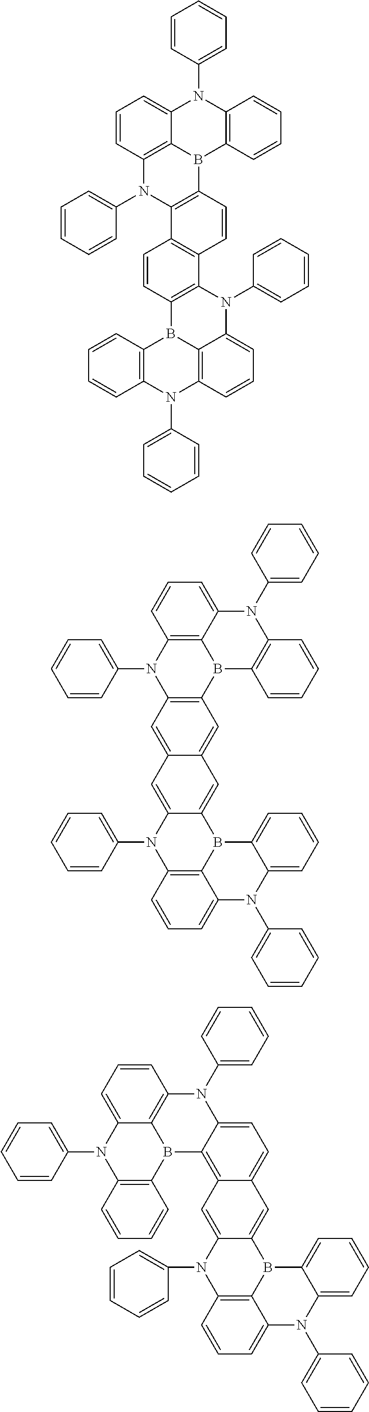

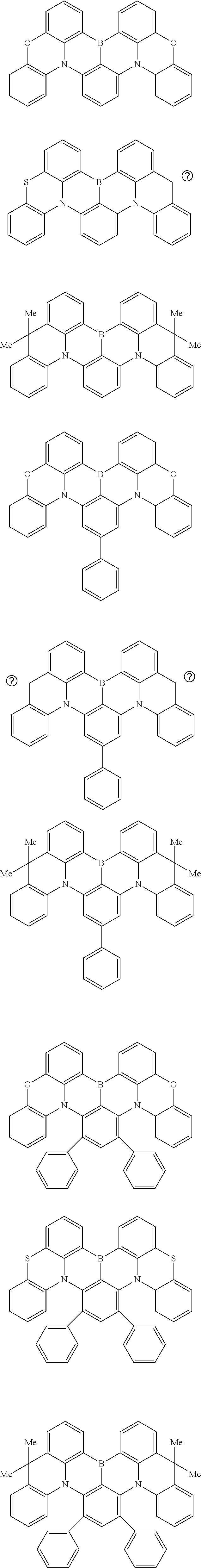

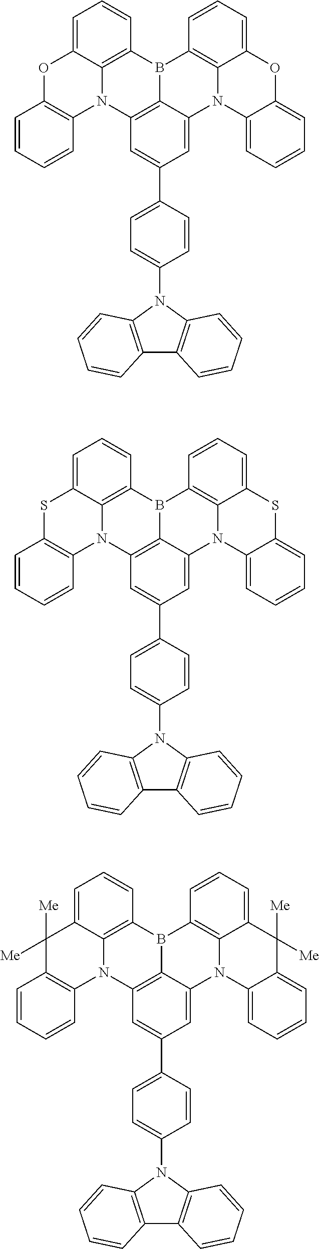 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C]

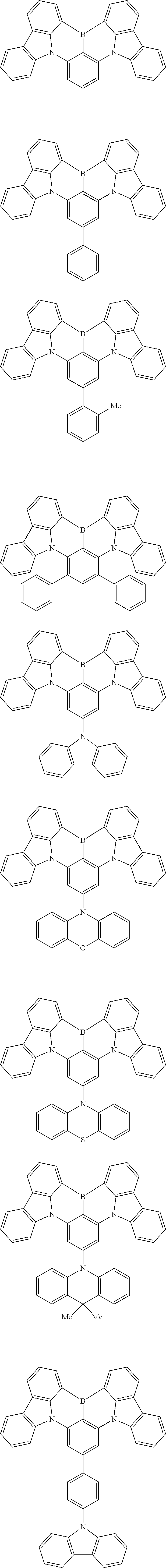

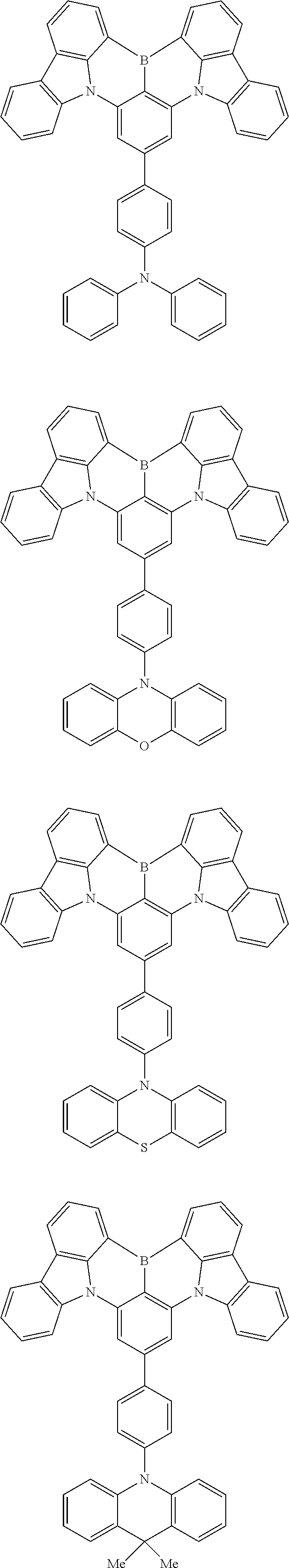 [SMILES: c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

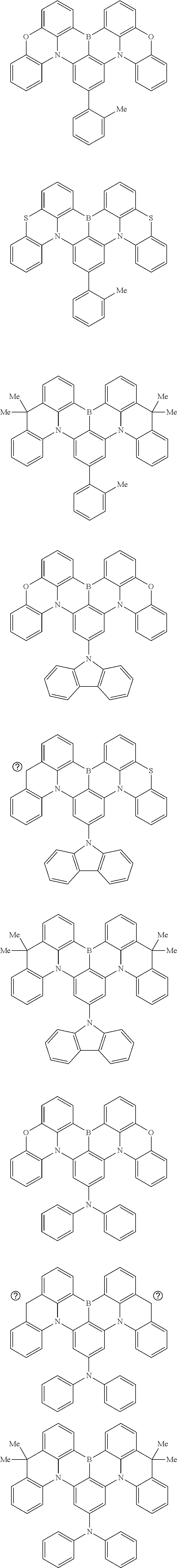

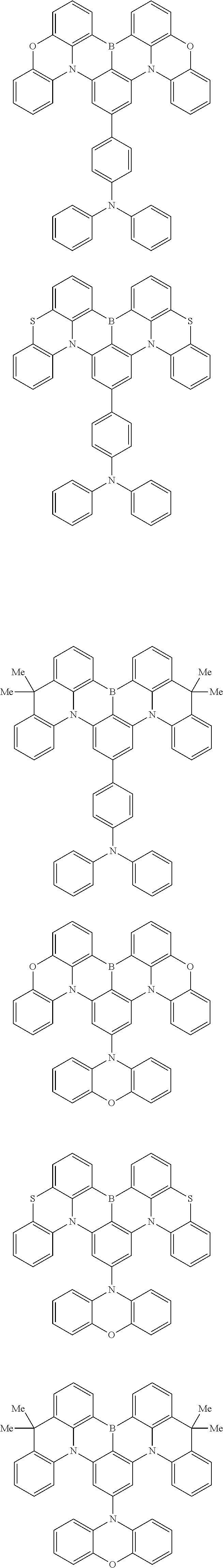

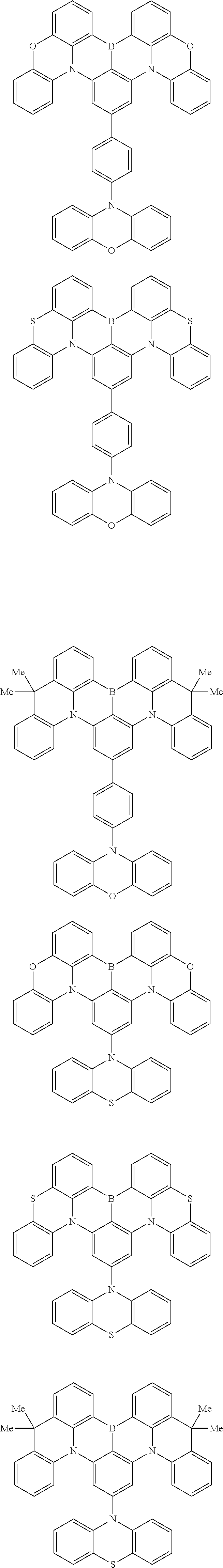

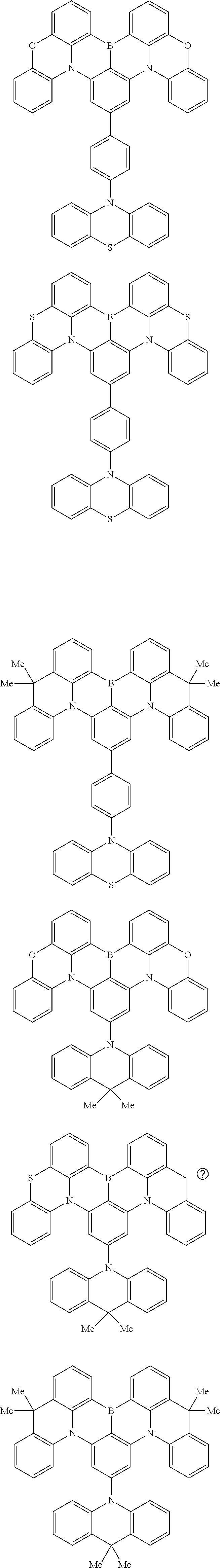

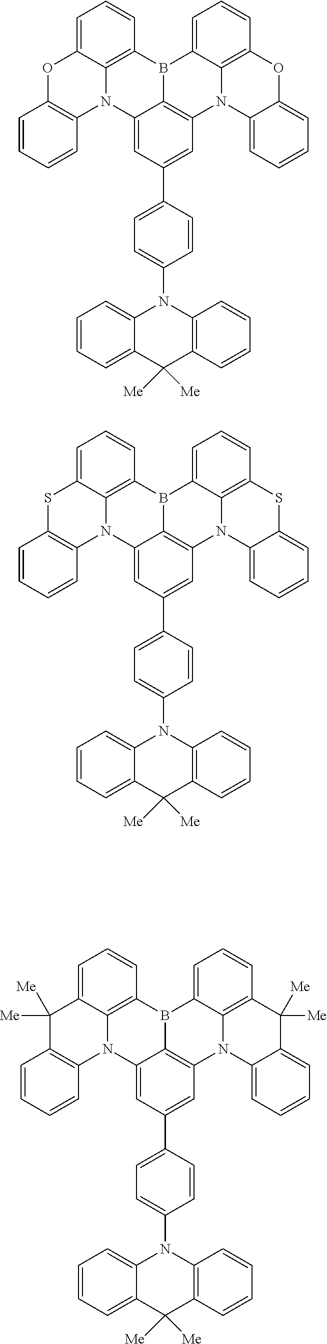 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21]

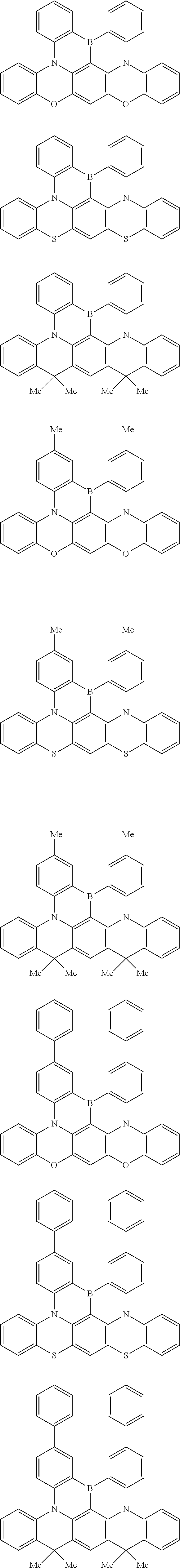

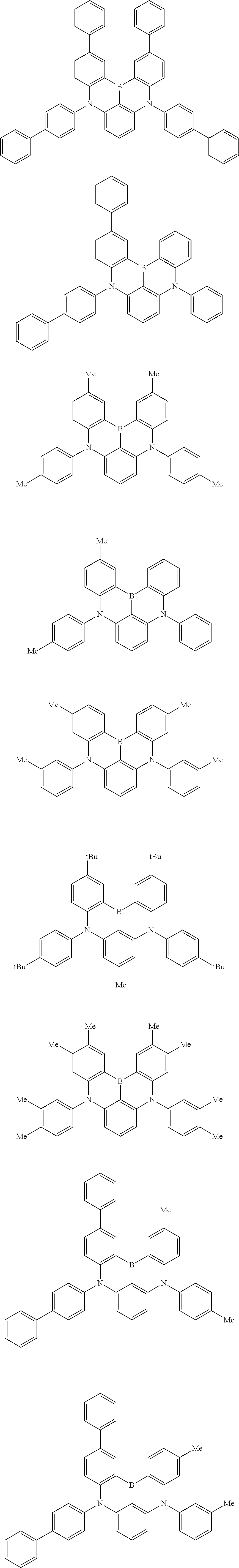

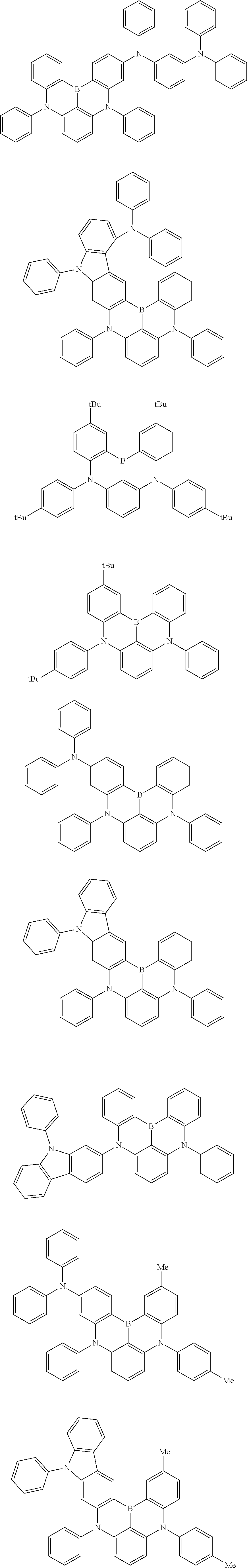

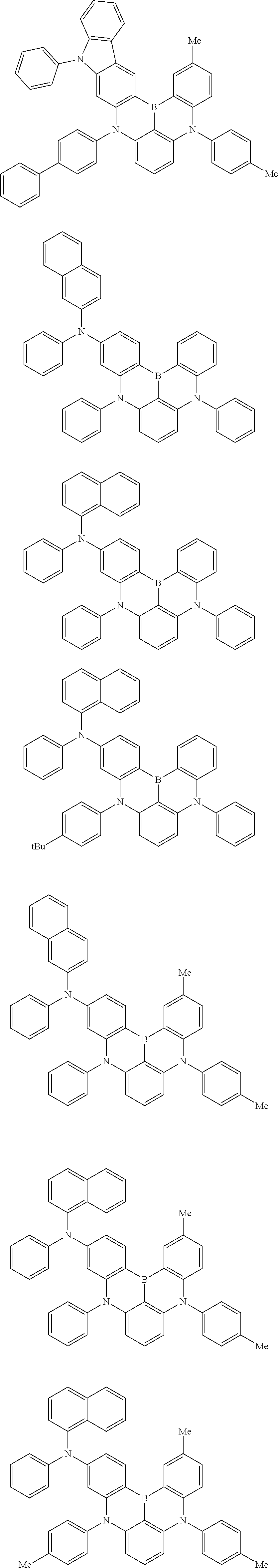

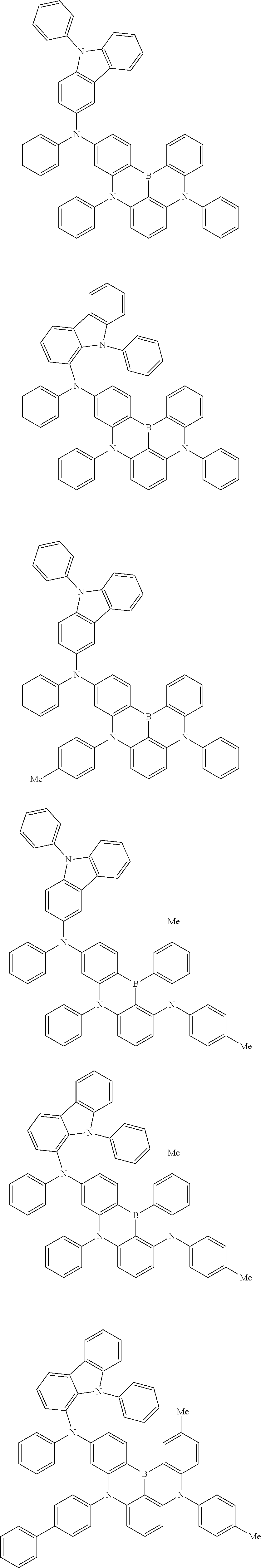

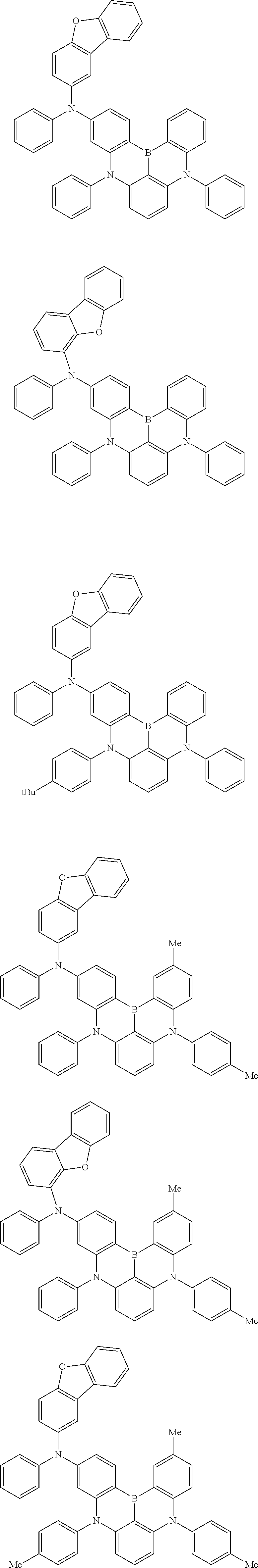

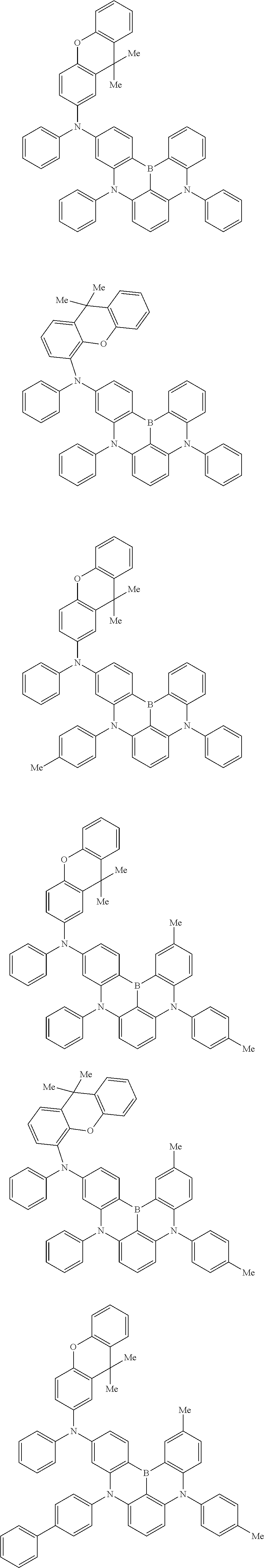

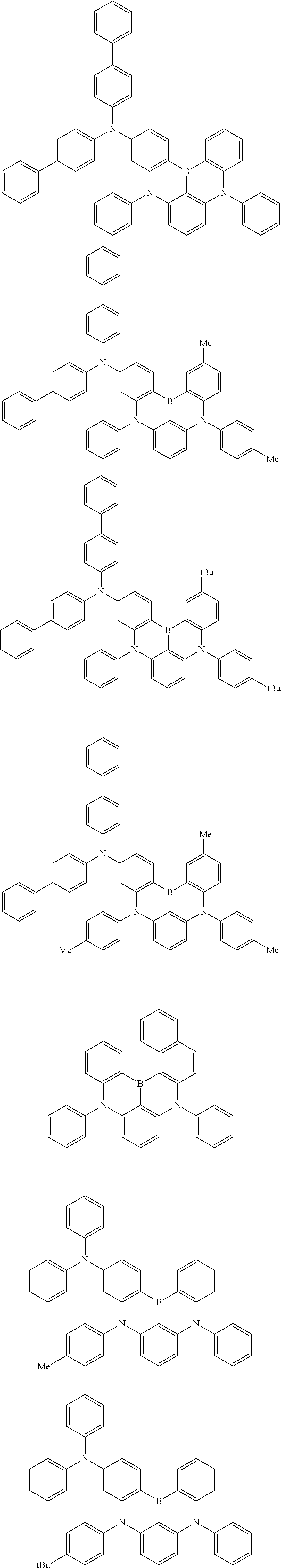

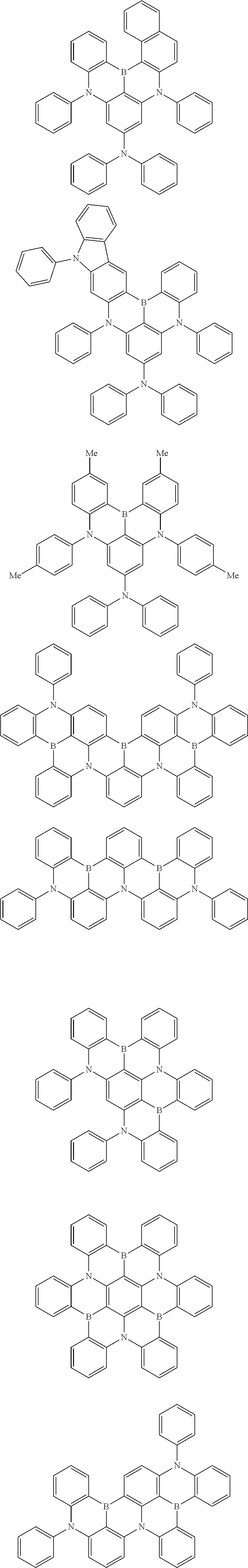

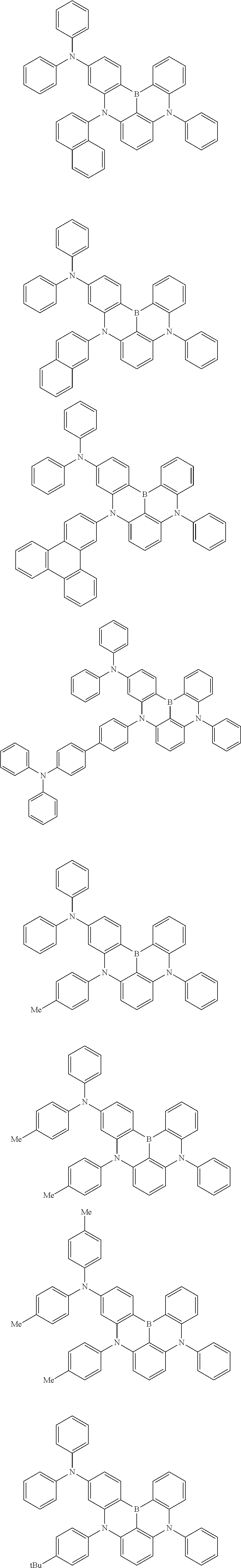

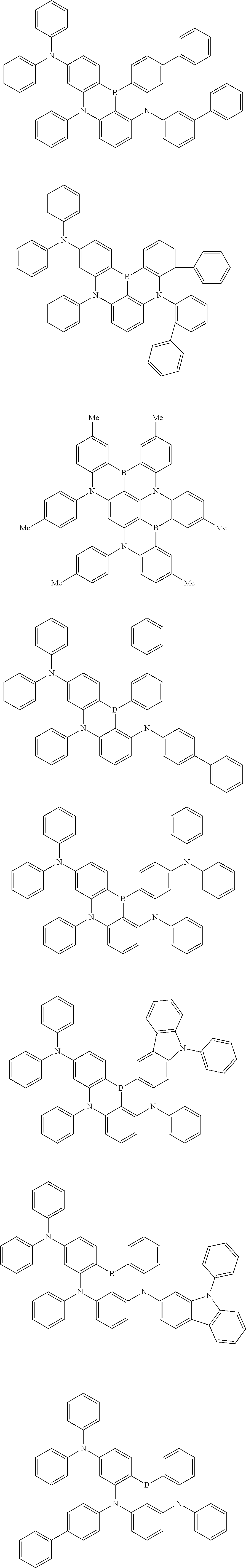

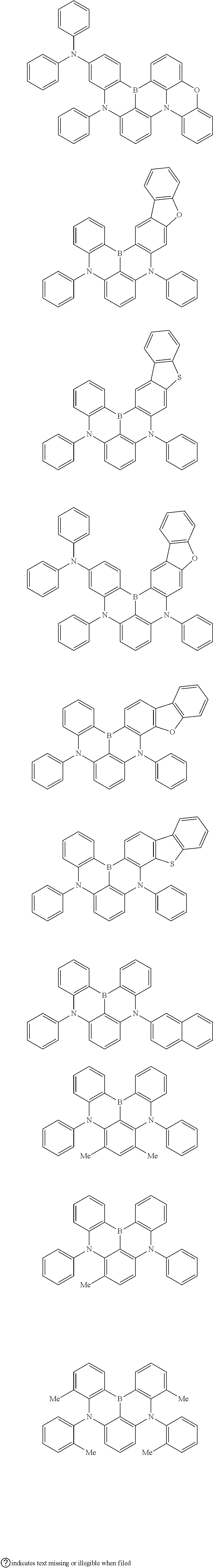

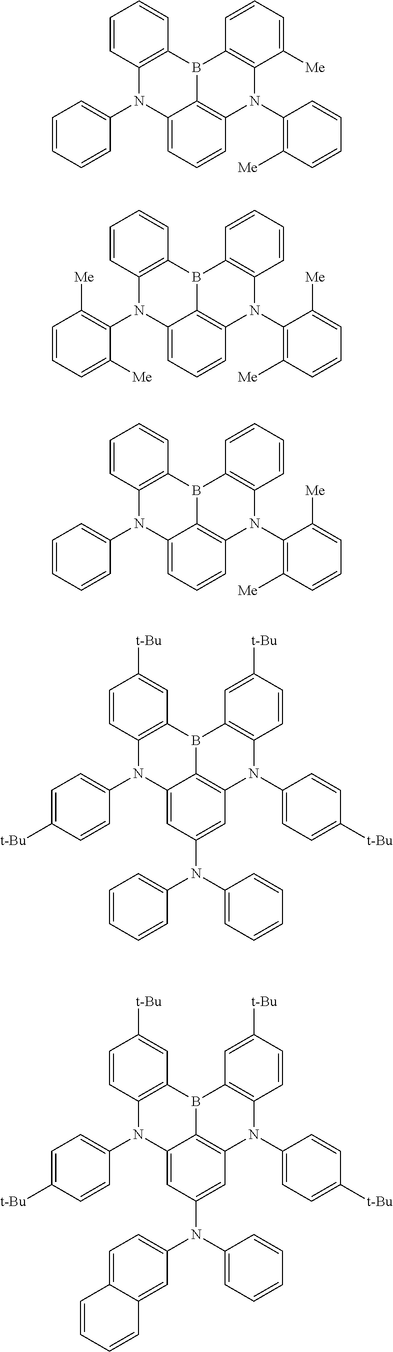

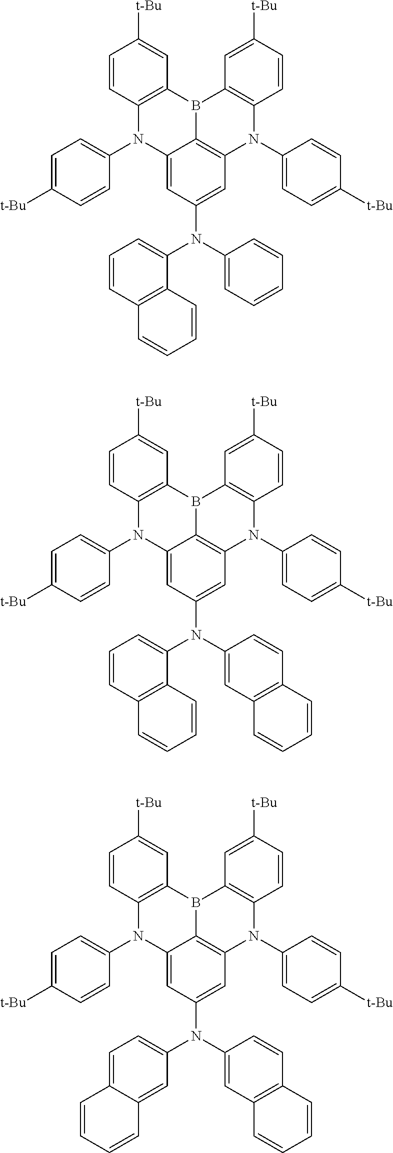 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1]

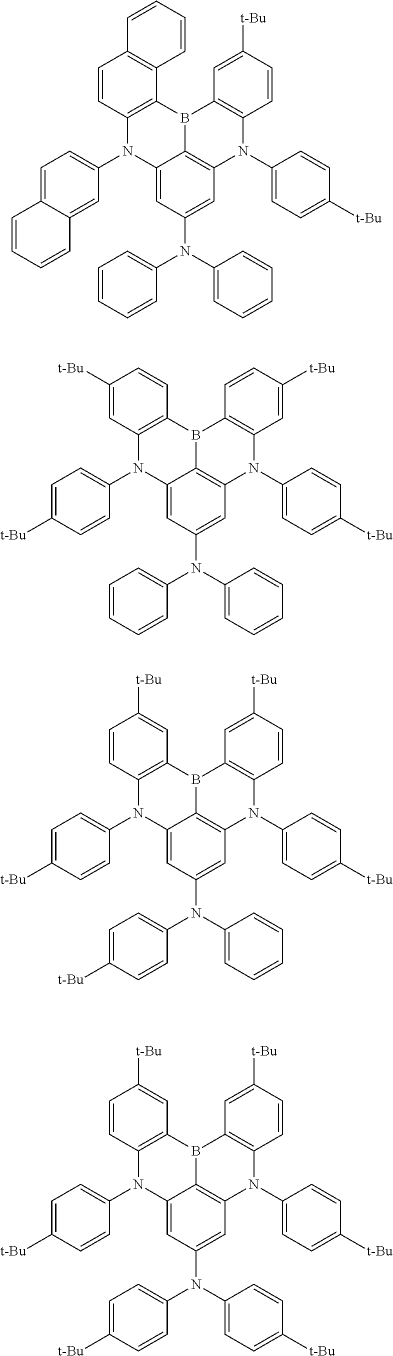

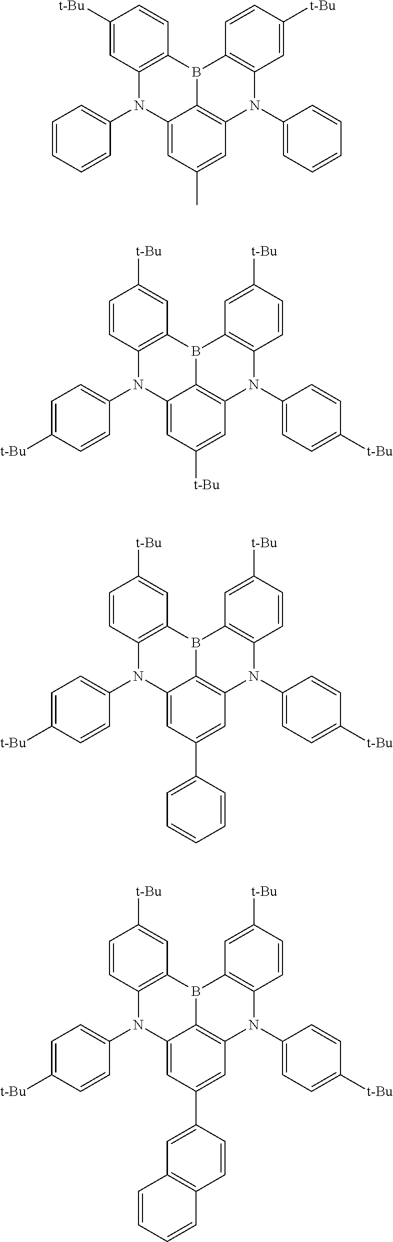 [SMILES: Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1]

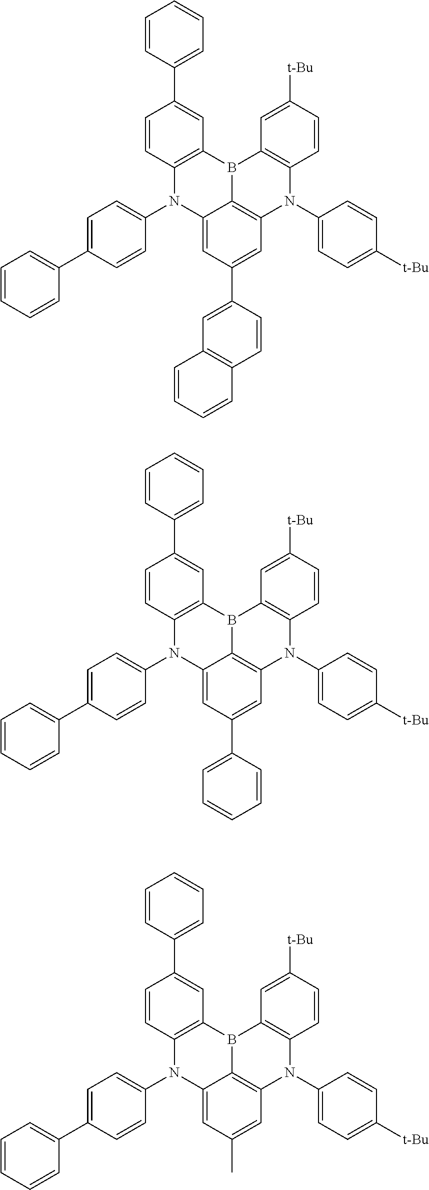

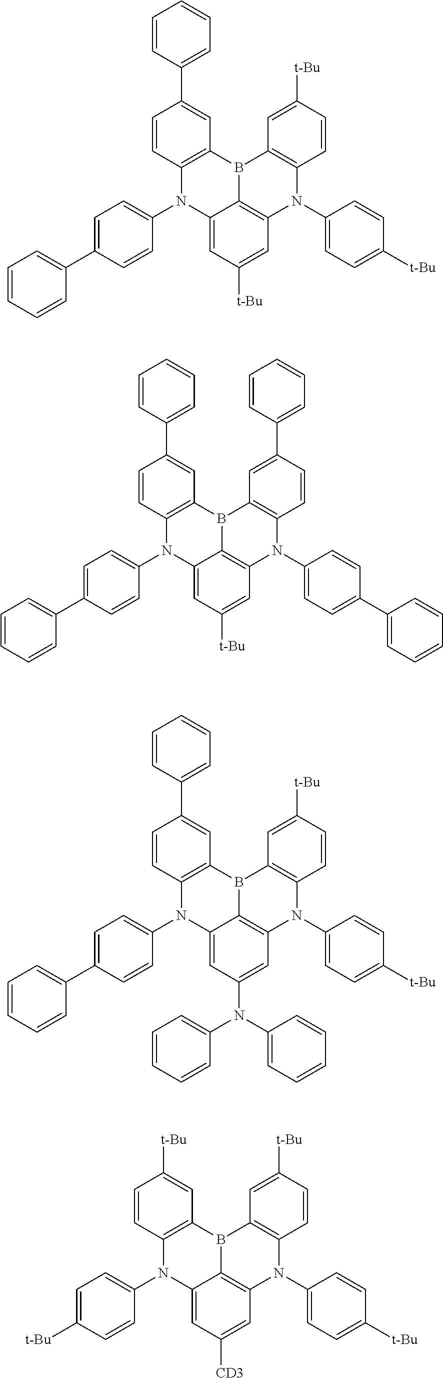 [SMILES: CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1 ; [2H]C([2H])([2H])c1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

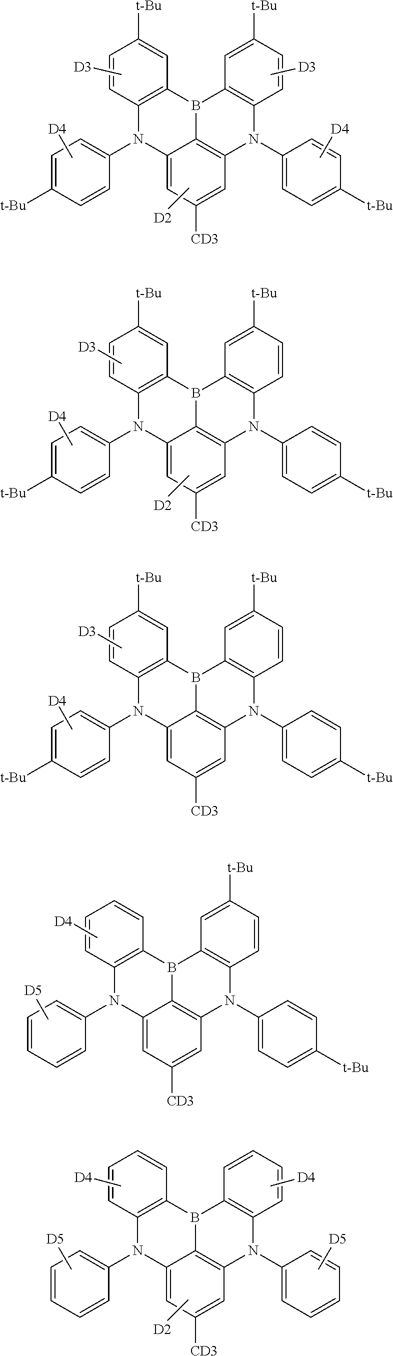

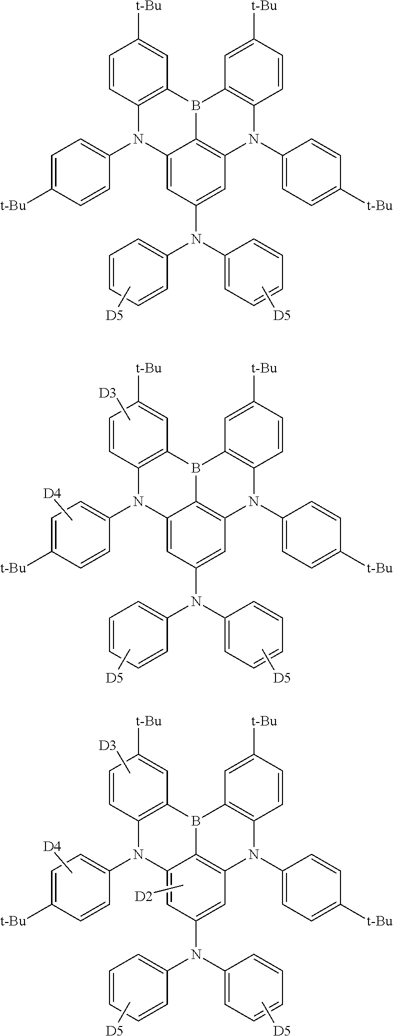

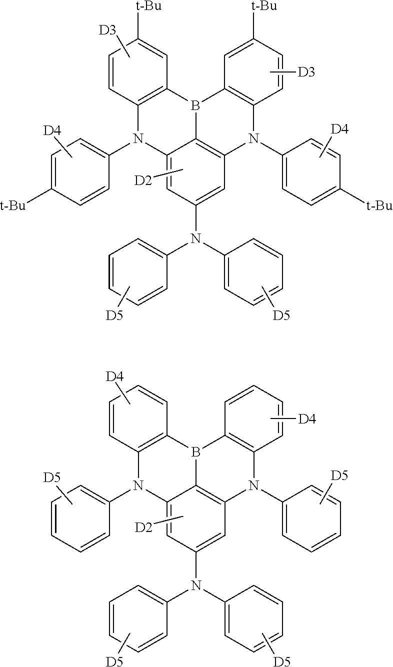

In the compounds, D2 to D5 indicate that 2 to 5 hydrogens are replaced by deuterium, respectively.

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability. When forming a layer by the organic electroluminescent compound according to one embodiment, the layer can be formed by way of the above-listed methods, and can often be formed by co-deposition or mixture-deposition. The co-deposition is a mixed deposition method in which two or more materials are put into respective individual crucible sources, and a current is applied to both cells simultaneously to evaporate the materials; the mixed deposition is a method in which two or more materials are mixed in one crucible source before the deposition, and then a current is applied to one cell to evaporate the materials.

According to one embodiment, the present disclosure can provide display devices comprising an organic electroluminescent compound represented by Formula 1 and organic electroluminescent compound represented by Formula 2. In addition, by using the organic electroluminescent device of the present disclosure, display devices such as smartphones, tablets, notebooks, PCs, TVs, or display devices for vehicles, or lighting devices such as outdoor or indoor lighting can be prepared. Hereinafter, the preparation method of the compound according to the present disclosure will be explained with reference to the synthesis method of a representative compound or intermediate compound in order to understand the present disclosure in detail.

### [Example 1] Preparation of Compound C-2

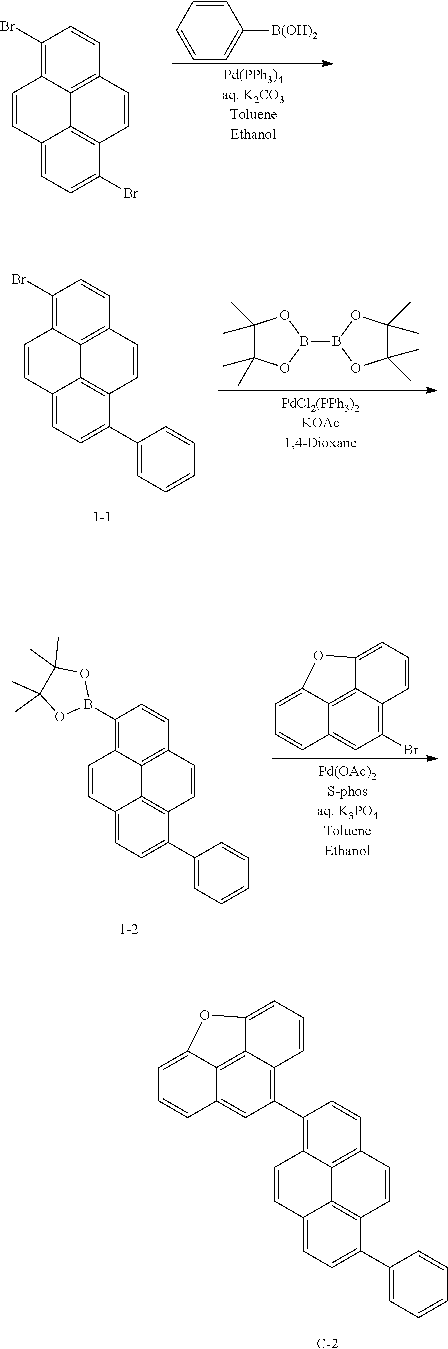

### 1) Synthesis of Compound 1-1

1,6-Dibromopyrene (50 g, 138.86 mmol), phenylboronic acid (20.31 g, 166.64 mmol), Pd(PPh₃)₄ (6.41 g, 5.55 mmol), 1,000 mL of toluene, K₂CO₃ (38.38 g, 277.73 mmol), 300 mL of distilled water, and 100 mL of ethanol were added to a flask and stirred under reflux. After 40 minutes, the reaction mixture was cooled to room temperature. Distilled water was then added to the reaction mixture, the organic layer was extracted with ethyl acetate, and this was then treated with MgSO₄.

Thereafter, the organic layer was filtered and concentrated, and the resulting mixture was purified by column chromatography to obtain Compound 1-1 (27 g, 54.4%).

### 2) Synthesis of Compound 1-2

Compound 1-1 (26 g, 72.77 mmol), PdCl₂(PPh₃)₂ (2.55 g, 3.63 mmol), 4,4,4′,4′,5,5,5′,5′-octamethyl-2,2′-bi(1,3,2-dioxaborolane) (33.26 g, 131.00 mmol), KOAc (17.90 g, 181.94 mmol), and 700 mL of 1,4-dioxane were added to a flask and stirred under reflux at 160° C. After 2 hours, the reaction mixture was cooled to room temperature, the organic layer was extracted with ethyl acetate, and this was then treated with MgSO₄. The organic layer was then filtered and concentrated, and the resulting mixture was purified by column chromatography to obtain Compound 1-2 (16 g, 54.38%).

### 3) Synthesis of Compound C-2

Compound 1-2 (16 g, 39.57 mmol), 8-bromophenanthro[4,5-bcd]furan (11.8 g, 43.52 mmol), Pd(OAc)₂ (0.35 g, 1.58 mmol), SPhos (1.95 g, 4.74 mmol), K₃PO₄ (21.0 g, 98.93 mmol), 400 mL of toluene, 100 mL of distilled water, and 50 mL of ethanol were added to a flask and stirred under reflux at 120° C. After 2 hours and 30 minutes, the reaction mixture was cooled to room temperature, distilled water was added to the reaction mixture, and the organic layer was extracted with ethyl acetate. Thereafter, the organic layer was treated with MgSO₄, filtered, and then concentrated, and the resulting mixture was purified by column chromatography to obtain Compound C-2 (16 g, 86.29%).

| | MW | M.P. |
|---|---|---|
| C-2 | 468.56 | 237° C. |

### [Example 2] Preparation of Compound C-467-D16

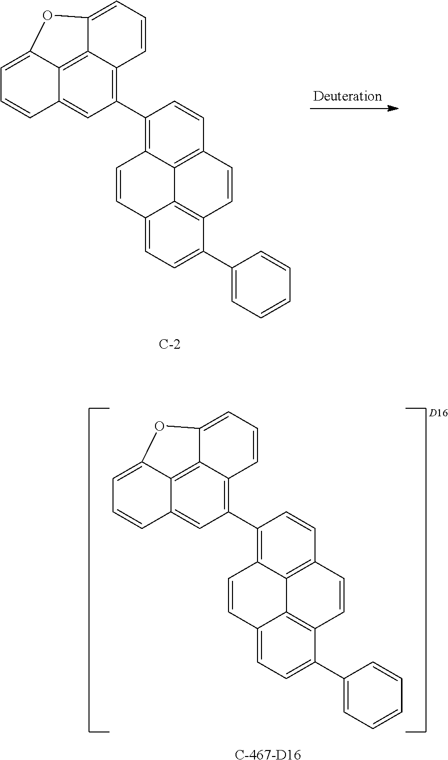

Compound C-2 was synthesized by selecting the deuteration methods disclosed in Korean Patent Nos. 10-2283849, 10-1427457, etc., and Compound C-467-D16 (7.6 g, yield: 55.51%, MS: [M+H]⁺=485.1) was obtained.

| | MW | M.P. |
|---|---|---|
| C-467-D16 | 484.1 | 232° C. |

### [Example 3] Preparation of Compound C-991

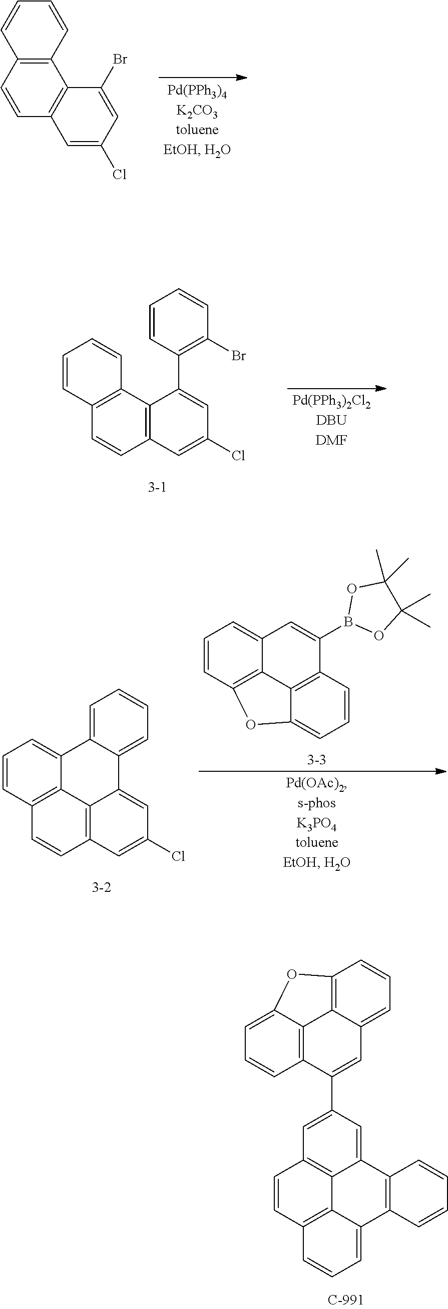 [SMILES: Clc1cc(Br)c2c(ccc3ccccc32)c1 ; Clc1cc(-c2ccccc2Br)c2c(ccc3ccccc32)c1 ; Clc1cc2ccc3cccc4c5ccccc5c(c1)c2c34 ; CC1(C)OB(c2cc3cccc4oc5cccc2c5c34)OC1(C)C ; c1cc2cc(-c3cc4ccc5cccc6c7ccccc7c(c3)c4c56)c3cccc4oc(c1)c2c43]

### 1) Synthesis of Compound 3-1

4-Bromo-2-chlorophenanthrene (30 g, 103 mmol), 2-bromophenylboronic acid (22.7 g, 113 mmol), Pd(PPh₃)₄ (5.9 g, 5.0 mmol), K₂CO₃ (27.2 g, 257 mmol), 520 mL of toluene, 130 mL of ethanol, and 130 mL of distilled water were added to a flask and dissolved. The reaction mixture was then refluxed at 120° C. for 4 hours. After the reaction was complete, the organic layer was extracted with ethyl acetate, dried of remaining moisture using magnesium sulfate, and then separated using column chromatography to obtain Compound 3-1 (37.8 g, yield: 100%).

### 2) Synthesis of Compound 3-2

Compound 3-1 (37.8 g, 102 mmol), Pd(PPh₃)₂Cl₂ (7.2 g, 10 mmol), and 1,8-diazabicyclo[5.4.0]undec-7-ene (DBU) (77 mL, 514 mmol) were added to 510 ml of DMF and then dissolved. The reaction mixture was then refluxed at 165° C. for 4 hours. After the reaction was complete, the organic layer was extracted with ethyl acetate, dried of remaining moisture using magnesium sulfate, and then separated using column chromatography to obtain Compound 3-2 (4 g, yield: 13.5%).

### 3) Synthesis of Compound C-991

Compound 3-2 (7.9 g, 27.5 mmol), Compound 3-3 (8.8 g, 27.5 mmol), Pd(OAc)₂ (309 mg, 1.38 mmol), SPhos (1.1 g, 2.75 mmol), K₃PO₄ (14.6 g, 69 mmol), toluene (140 mL), ethanol (35 mL), and distilled water (35 mL) were added to a flask and refluxed at 120° C. for 3 hours. After the reaction was complete, the organic layer was extracted with ethyl acetate, dried of remaining moisture using magnesium sulfate, and then separated using column chromatography to obtain Compound C-991 (8.4 g, yield: 69%).

| | MW | M.P. |
|---|---|---|
| C-991 | 442.52 | 277° C. |

Hereinafter, the preparation method of an organic electroluminescent device comprising the organic electroluminescent compound according to the present disclosure and the device properties thereof will be explained in order to aid in understanding the present disclosure in detail.

[Device Examples 1 to 3] Preparation of OLEDs Comprising a Plurality of Light-Emitting Layers Deposited with a Compound According to the Present Disclosure

OLEDs according to the present disclosure were prepared. First, a transparent electrode indium tin oxide (ITO) thin film on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-3 was introduced into another cell. The two materials were evaporated at different rates, and Compound HI-1 was deposited in a doping amount of 5 wt % based on the total amount of Compounds HI-1 and HT-3 to form a hole injection layer having a thickness of 10 nm. Next, Compound HT-3 was deposited as a first hole transport layer having a thickness of 80 nm on the hole injection layer. Compound HT-4 was then introduced into another cell of the vacuum vapor deposition apparatus and was evaporated by applying an electric current to the cell, thereby forming a second hole transport layer having a thickness of 15 nm on the first hole transport layer. After formation of the hole injection layer and the hole transport layers, a first light-emitting layer was formed thereon as follows: The compound described in Table 1 below was introduced into a cell of the vacuum vapor deposition apparatus as a host, and Compound D-2 was introduced into another cell as a dopant. The two materials were evaporated at different rates, and were deposited in a doping amount of 2 wt % based on the total amount of the host and dopant to form a first light-emitting layer having a thickness of 5 nm on the second hole transport layer. Next, a second light-emitting layer was formed on the first light-emitting layer as follows: The compound described in Table 1 below was introduced into a cell of the vacuum vapor deposition apparatus as a host, and Compound D-2 was introduced into another cell as a dopant. The two materials were evaporated at different rates, and were deposited in a doping amount of 2 wt % based on the total amount of the host and dopant to form a second light-emitting layer having a thickness of 13 nm on the first light-emitting layer. After formation of the light-emitting layers, Compound ET-1 was deposited as a hole-blocking layer material to form a hole-blocking layer with a thickness of 5 nm. Compound ET-3 and Compound EI-1 were then introduced into two cells of the vacuum vapor deposition apparatus as electron transport layer materials, respectively, and the two materials were deposited at a ratio of 2:1 to form an electron transport layer having a thickness of 25 nm. Next, Yb and LiF were evaporated at a ratio of 2:1 to form an electron injection layer having a thickness of 1 nm on the electron transport layer. Thereafter, an Al cathode having a thickness of 80 nm was deposited on the electron injection layer by another vacuum vapor deposition apparatus. Thus, OLEDs were produced. Each of the compounds used for all of the materials were purified by vacuum sublimation at 10⁻⁶ Torr.

### [Device Comparative Example 1] Preparation of an OLED Comprising a Single Light-Emitting Layer

An OLED was manufactured in the same manner as Device Example 1, except that a light-emitting layer having a thickness of 18 nm was formed by adding Compound H3-11 of Table 1 below as a host material of a single light-emitting layer, adding Compound D-2 as a dopant, and then doping the dopant in an amount of 2 wt % based on the total amount of the host and dopant.

The driving voltage at a luminance of 1,000 nits and the current efficiency of the OLEDs of Device Examples 1 to 3 and Device Comparative Example 1 produced as described above were measured, and the results thereof are shown in Table 1 below.

TABLE 1

| | First Light- | Second Light- | Driving | Current |
|---|---|---|---|---|
| | Emitting | Emitting | Voltage | Efficiency |
| | Layer Host | Layer Host | [V] | [cd/A] |
| Device Example 1 | C-991 | H3-11 | 3.7 | 6.1 |
| Device Example 2 | C-271 | H3-11 | 3.6 | 6.1 |
| Device Example 3 | C-281 | H3-11 | 3.6 | 5.7 |
| Device | H3-11 | | 3.7 | 5.6 |
| Comparative | | | | |
| Example 1 | | | | |

Referring to Table 1 above, it can be confirmed that an organic electroluminescent device including a plurality of light-emitting layers, each including an organic electroluminescent compound according to the present invention, has lower driving voltage and/or higher efficiency characteristics compared to an organic electroluminescent device including only a single light-emitting layer.

The compounds used in Device Examples 1 to 3 and Device Comparative Example 1 are specifically shown in Table 2 below.

TABLE 2

| | |
|---|---|
| Hole Injection Layer/Hole Transport Layer | 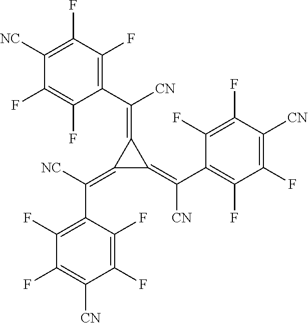 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 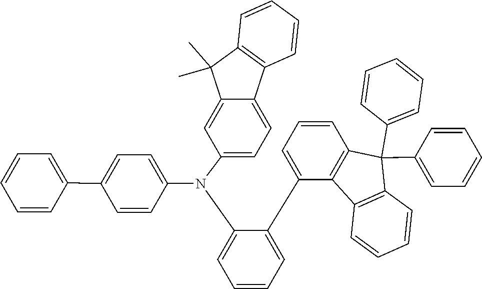 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-3 |
| | 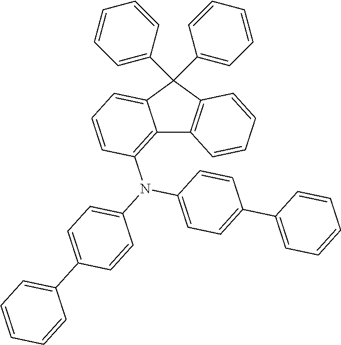 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-4 |
| Light- Emitting Layer | 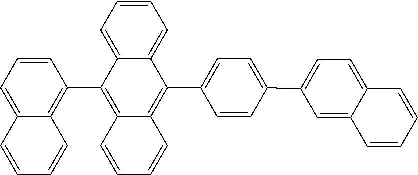 [SMILES: c1ccc2cc(-c3ccc(-c4c5ccccc5c(-c5cccc6ccccc56)c5ccccc45)cc3)ccc2c1] |
| | H3-11 |
| | 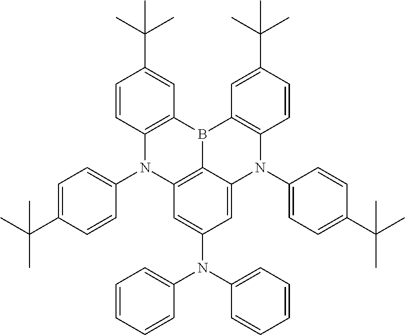 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | D-2 |
| | 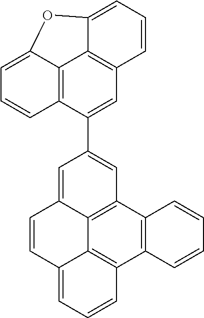 [SMILES: c1cc2cc(-c3cc4ccc5cccc6c7ccccc7c(c3)c4c56)c3cccc4oc(c1)c2c43] |
| | C-991 |
| | 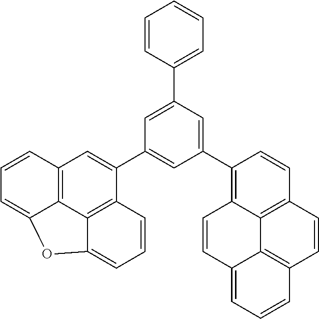 [SMILES: c1ccc(-c2cc(-c3ccc4ccc5cccc6ccc3c4c56)cc(-c3cc4cccc5oc6cccc3c6c45)c2)cc1] |
| | C-271 |
| | 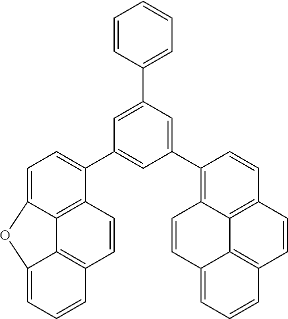 [SMILES: c1ccc(-c2cc(-c3ccc4ccc5cccc6ccc3c4c56)cc(-c3ccc4oc5cccc6ccc3c4c65)c2)cc1] |
| | C-281 |
| Electron Transport Layer/Hole- Blocking Layer/ Electron Injection Layer | 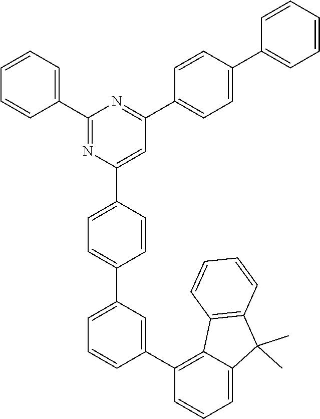 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 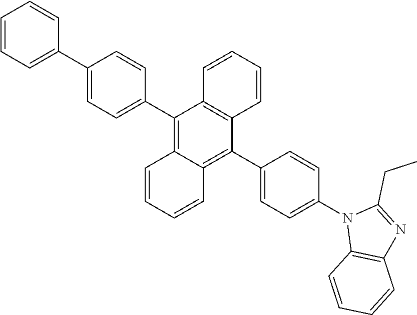 [SMILES: CCc1nc2ccccc2n1-c1ccc(-c2c3ccccc3c(-c3ccc(-c4ccccc4)cc3)c3ccccc23)cc1] |
| | ET-3 |
| | 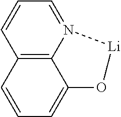 |
| | EI-1 |

[Device Examples 4 and 5] Preparation of OLEDs Comprising a Plurality of Light-Emitting Layers and an n-Type Charge Generation Layer Deposited with a Compound According to the Present Disclosure

OLEDs according to the present disclosure were prepared. First, a transparent electrode indium tin oxide (ITO) thin film on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a hole injection layer having a thickness of 5 nm. Next, Compound HT-1 was deposited as a first hole transport layer having a thickness of 30 nm on the hole injection layer. Compound HT-2 was then introduced into another cell of the vacuum vapor deposition apparatus and was evaporated by applying an electric current to the cell, thereby forming a second hole transport layer having a thickness of 5 nm on the first hole transport layer. After formation of the hole injection layer and the hole transport layers, a first light-emitting layer was formed thereon as follows: The compound described in Table 3 below was introduced into a cell of the vacuum vapor deposition apparatus as a host, and Compound D-1 was introduced into another cell as a dopant. The two materials were evaporated at different rates, and were deposited in a doping amount of 2 wt % based on the total amount of the host and dopant to form a first light-emitting layer having a thickness of 5 nm on the second hole transport layer. A second light-emitting layer was then formed on the first light-emitting layer as follows: The compound described in Table 3 below was introduced into a cell of the vacuum vapor deposition apparatus as a host, and Compound D-1 was introduced into another cell as a dopant. The two materials were evaporated at different rates, and were deposited in a doping amount of 2 wt % based on the total amount of the host and dopant to form a second light-emitting layer having a thickness of 15 nm on the first light-emitting layer. After deposition of the light-emitting layers, Compound ET-1 was deposited as a hole-blocking layer material to form a hole-blocking layer with a thickness of 5 nm. Compound ET-2 was then deposited as an electron transport layer material to form a first electron transport layer with a thickness of 10 nm. Thereafter, Li was deposited in a doping amount of 0.5 wt % to the compound of Table 3 below to form an n-type charge generation layer with a thickness of 4 nm. Compound HI-1 was then deposited in a doping amount of 6 wt % based on the total amount of Compound HI-1 and Compound HT-3 to form a p-type charge generation layer with a thickness of 10 nm. Compound HT-3 was deposited thereon to form a third hole transport layer having a thickness of 30 nm, and then Compound HT-4 was deposited to form a fourth hole transport layer having a thickness of 5 nm. Thereafter, a third light-emitting layer was deposited thereon as follows: The compound of Table 3 as a host was introduced into a cell of a vacuum deposition device, and Compound D-1 was introduced as a dopant, and the two materials were evaporated at different rates and were deposited in a doping amount of 2 wt % based on the total amount of the host and the dopant to form a third light-emitting layer with a thickness of 5 nm on the fourth hole transport layer. A fourth light-emitting layer was deposited on the third light-emitting layer as follows: The compound of Table 3 as a host was introduced into a cell of a vacuum deposition device, and Compound D-1 was added as a dopant, and the two materials were evaporated at different rates and were deposited in a doping amount of 2 wt % based on the total amount of the host and the dopant to form a fourth light-emitting layer with a thickness of 15 nm on the fourth hole transport layer. Compound ET-1 was then deposited as a second hole-blocking layer material thereon to form a hole-blocking layer with a thickness of 5 nm, and Compounds ET-2 and EI-1 were added to two cells of a vacuum deposition device as second electron transport layer materials, respectively, and the two materials were deposited at a weight ratio of 2:1 to deposit a second electron transport layer having a thickness of 25 nm. After deposition of Yb as an electron injection layer with a thickness of 1 nm on the second electron transport layer, an Al cathode having a thickness of 80 nm was deposited on the electron injection layer by another vacuum vapor deposition apparatus. Thus, OLEDs were produced. Each of the compounds used for all of the materials were purified by vacuum sublimation at 10⁻⁶ Torr.

### [Device Comparative Example 2] Preparation of an OLED Comprising a Single Light-Emitting Layer

An OLED was manufactured in the same manner as in Device Example 4, except that instead of the first light-emitting layer and the second light-emitting layer, the compound of Table 3 below was added as a host material, and Compound D-1 was deposited as a dopant in a doping amount of 2 wt % based on the total amount of the host and the dopant, thereby depositing a first light-emitting layer having a thickness of 20 nm, and instead of the third light-emitting layer and the fourth light-emitting layer, the compound of Table 3 below was added as a host material of the second light-emitting layer, and the deposition was performed in the same manner as for the first light-emitting layer.

The driving voltage at a luminance of 1,000 nits, the luminous efficacy, and the time taken for the light intensity to decrease from 100% to 95% when checking the lifespan with 2× acceleration (lifespan; T₉₅) of the OLEDs of Device Examples 4 and 5 and Device Comparative Example 2 produced as described above were measured, and the results thereof are shown in Table 3 below.

TABLE 3

| | First | Second | n-type | Third | Fourth | | | |
|---|---|---|---|---|---|---|---|---|
| | Light- | Light- | Charge | Light- | Light- | | | |
| | Emitting | Emitting | Generation | Emitting | Emitting | Diving | Current | |
| | Layer | Layer | Layer | Layer | Layer | Voltage | Efficiency | Lifespan |
| | Host | Host | Material | Host | Host | [V] | [cd/A] | T₉₅ [hr] |
| Device | C-57 | H3-11 | G-301 | C-2 | H3-11 | 7.2 | 9.0 | 313 |
| Example 4 | | | | | | | | |
| Device | C-522- | H3-11 | G-301 | C-467- | H3-11 | 7.2 | 8.9 | 502 |
| Example 5 | D16 | | | D16 | | | | |
| Device | H3-11 | | G-301 | H3-11 | | 7.6 | 8.6 | 224 |
| Comparative | | | | | | | | |
| Example 2 | | | | | | | | |

Referring to Table 3 above, it can be confirmed that an organic electroluminescent device including at least two light-emitting units having a plurality of light-emitting layers according to the present disclosure has lower driving voltage and/or higher efficiency and/or longer lifespan characteristics compared to an organic electroluminescent device including only a single light-emitting layer.

The compounds used in Device Examples 4 and 5 and Device Comparative Example 2 are specifically shown in Table 4 below.

TABLE 4

| | |
|---|---|
| Hole Injection Layer/Hole Transport Layer | 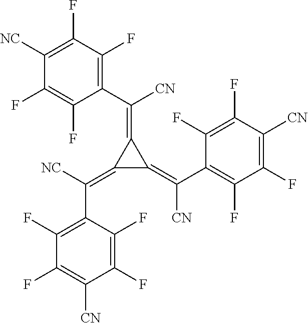 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 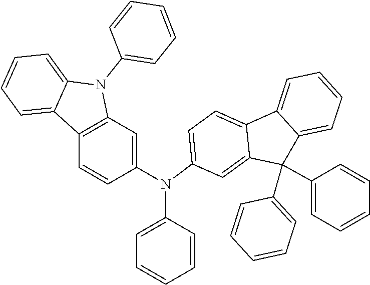 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-1 |
| | 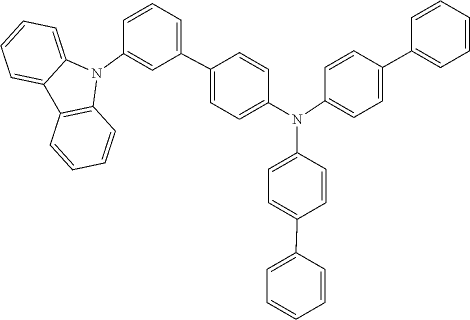 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-2 |
| Light- Emitting Layer | 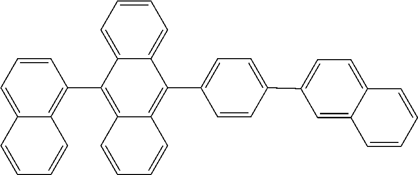 [SMILES: c1ccc2cc(-c3ccc(-c4c5ccccc5c(-c5cccc6ccccc56)c5ccccc45)cc3)ccc2c1] |
| | H3-11 |
| | 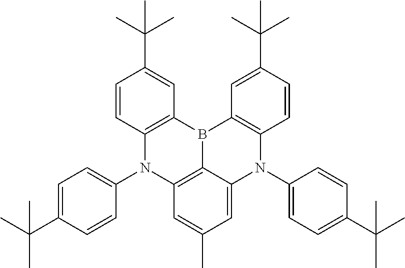 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | D-1 |
| | 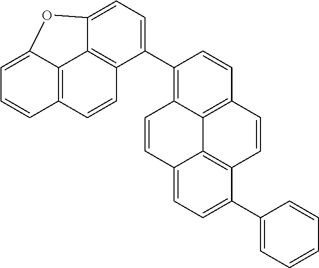 [SMILES: c1ccc(-c2ccc3ccc4c(-c5ccc6oc7cccc8ccc5c6c87)ccc5ccc2c3c54)cc1] |
| | C-57 |
| | 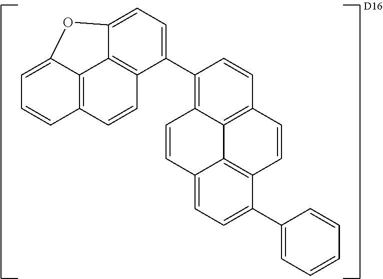 |
| | C-522-D16 |
| | 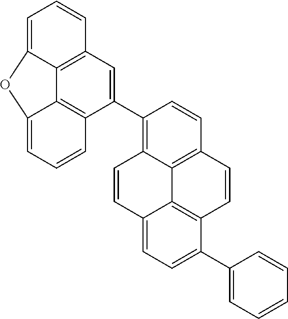 [SMILES: c1ccc(-c2ccc3ccc4c(-c5cc6cccc7oc8cccc5c8c67)ccc5ccc2c3c54)cc1] |
| | C-2 |
| | 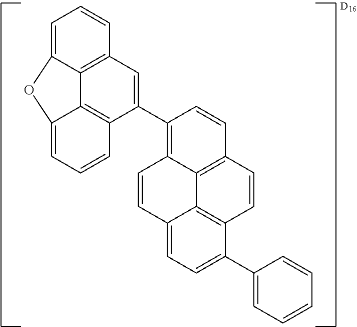 |
| | C-467-D16 |
| Hole-Blocking Layer/ Electron Transport Layer/ Electron Injection Layer | 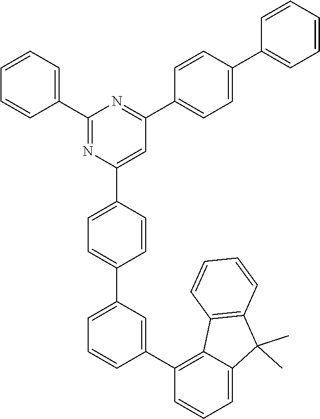 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 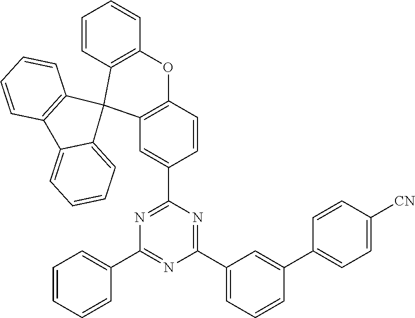 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-2 |
| | 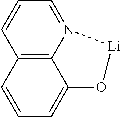 |
| | EI-1 |
| n-Type Charge Generation Layer | 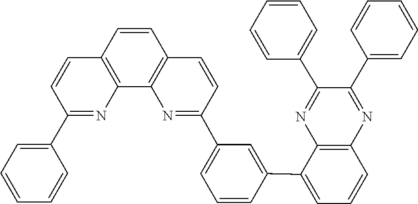 [SMILES: c1ccc(-c2ccc3ccc4ccc(-c5cccc(-c6cccc7nc(-c8ccccc8)c(-c8ccccc8)nc67)c5)nc4c3n2)cc1] |
| | G-301 |

[Device Example 6] Preparation of an OLED Comprising a Plurality of Light-Emitting Layers Deposited with a Compound According to the Present Disclosure

An OLED according to the present disclosure was prepared. First, a transparent electrode indium tin oxide (ITO) thin film on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-3 was introduced into another cell. The two materials were evaporated at different rates, and Compound HI-1 was deposited in a doping amount of 5 wt % based on the total amount of Compounds HI-1 and HT-3 to form a hole injection layer having a thickness of 10 nm. Next, Compound HT-3 was deposited as a first hole transport layer having a thickness of 80 nm on the hole injection layer. Compound HT-4 was then introduced into another cell of the vacuum vapor deposition apparatus and was evaporated by applying an electric current to the cell, thereby forming a second hole transport layer having a thickness of 15 nm on the first hole transport layer. After formation of the hole injection layer and the hole transport layers, a first light-emitting layer was formed thereon as follows: The compound described in Table 5 below was introduced into a cell of the vacuum vapor deposition apparatus as a host, and Compound D-2 was introduced into another cell as a dopant. The two materials were evaporated at different rates, and were deposited in a doping amount of 2 wt % based on the total amount of the host and dopant to form a first light-emitting layer having a thickness of 5 nm on the second hole transport layer. A second light-emitting layer was then formed on the first light-emitting layer as follows: The compound described in Table 5 below was introduced into a cell of the vacuum vapor deposition apparatus as a host, and Compound D-2 was introduced into another cell as a dopant. The two materials were evaporated at different rates, and were deposited in a doping amount of 2 wt % based on the total amount of the host and dopant to form a second light-emitting layer having a thickness of 13 nm on the first light-emitting layer. After deposition of the light-emitting layers, Compound ET-1 was deposited as a hole-blocking layer material to form a hole-blocking layer with a thickness of 5 nm. Compound ET-3 and Compound EI-1 were then introduced into two cells of the vacuum vapor deposition apparatus as electron transport layer materials, respectively, and the two materials were evaporated at a ratio of 2:1 to deposit an electron transport layer having a thickness of 25 nm. Thereafter, Yb and LiF were deposited as an electron injection layer material at a ratio of 2:1 to form an electron injection layer having a thickness of 1 nm on the electron transport layer. An Al cathode having a thickness of 80 nm was then deposited on the electron injection layer by another vacuum vapor deposition apparatus. Thus, an OLED was produced. Each of the compounds used for all of the materials were purified by vacuum sublimation at 10⁻⁶ Torr.

[Device Example 7] Preparation of an OLED Comprising a Plurality of Light-Emitting Layers Deposited with a Compound According to the Present Disclosure

An OLED was manufactured in the same manner as in Device Example 6, except that a third light-emitting layer having a thickness of 5 nm was formed on the second light-emitting layer by introducing the compound of Table 5 as a host into a cell of a vacuum deposition device, introducing Compound D-2 as a dopant into another cell, and then evaporating the two materials at different rates to deposit in a doping amount of 2 wt % based on the total amount of the host and the dopant.

[Device Comparative Example 3] Manufacturing of an OLED Deposited with a Compound According to the Present Disclosure as a Light-Emitting Layer

An OLED was manufactured in the same manner as Device Example 6, except that a single light-emitting layer having a thickness of 18 nm was formed by introducing the compound of Table 5 below as a host material, introducing Compound D-2 as a dopant to deposit in a doping amount of 2 wt % based on the total amount of the host and dopant.

The current efficiency at a luminance of 1,000 nits, the external quantum efficiency (EQE), and the time taken for the light intensity to decrease from 100% to 95% when checking the lifespan with 2× acceleration (lifespan; T₉₅) of the OLEDs of Device Examples 6 and 7 and Device Comparative Example 3 produced as described above were measured, and the results thereof are shown in Table 5 below.

TABLE 5

| | First Light- | Second Light- | Third Light- | Current | | |
|---|---|---|---|---|---|---|
| | Emitting | Emitting Layer | Emitting Layer | Efficiency | EQE | Lifespan |
| | Layer Host | Host | Host | [cd/A] | [%] | T₉₅ [hr] |
| Device | C-522-D16 | H2-291-D17 | — | 5.9 | 9.1 | 258 |
| Example 6 | | | | | | |
| Device | C-522-D16 | H2-291-D17 | C-522-D16 | 6.2 | 9.3 | 277 |
| Example 7 | | | | | | |
| Device | H3-11 | | | 4.8 | 8.3 | 141 |
| Comparative | | | | | | |
| Example 3 | | | | | | |

Referring to Table 5 above, it can be confirmed that an organic electroluminescent device including an organic electroluminescent compound according to the present disclosure in each of a plurality of light-emitting layers according to the present disclosure not only exhibits high current efficiency and/or high external quantum efficiency, but also has excellent lifespan characteristics, compared to an organic electroluminescent device including only a single light-emitting layer.

The compounds used in Device Examples 6 and 7 and Device Comparative Example 3 are specifically shown in Table 6 below.

TABLE 6

| | |
|---|---|
| Hole Injection Layer/Hole Transport Layer | 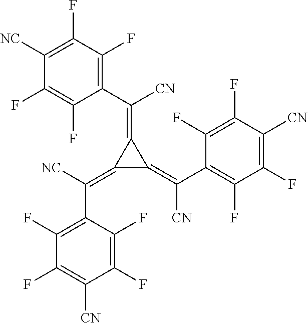 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 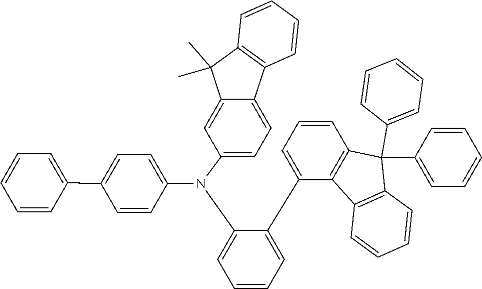 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-3 |
| | 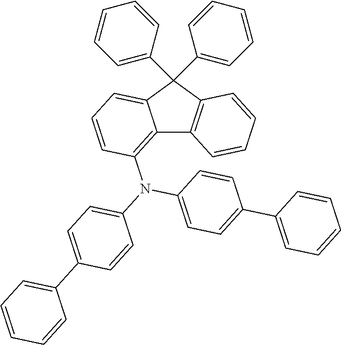 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-4 |
| Light-Emitting Layer | 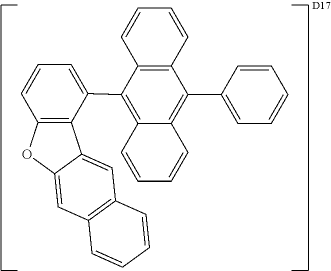 |
| | H2-921-D17 |
| | 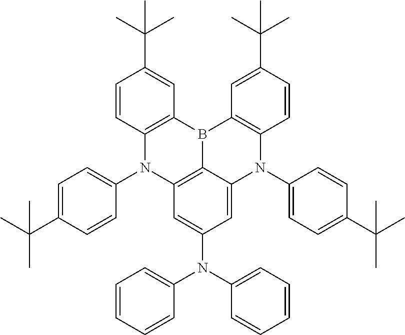 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | D-2 |
| | 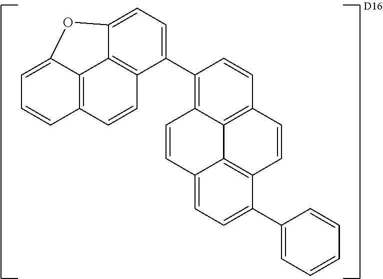 |
| | C-522-D16 |
| Hole-Blocking Layer/ Electron Transport Layer/ Electron Injection Layer | 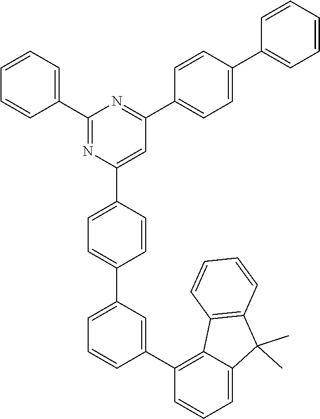 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 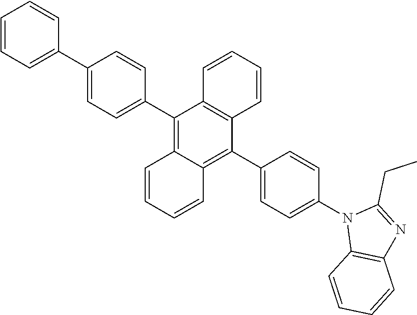 [SMILES: CCc1nc2ccccc2n1-c1ccc(-c2c3ccccc3c(-c3ccc(-c4ccccc4)cc3)c3ccccc23)cc1] |
| | ET-3 |
| | 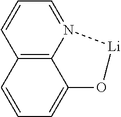 |
| | EI-1 |

[Device Examples 8 to 17] Preparation of OLEDs Deposited with a Compound According to the Present Disclosure as a Light-Emitting Layer

OLEDs according to the present disclosure were prepared. First, a transparent electrode indium tin oxide (ITO) thin film on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-3 was introduced into another cell. The two materials were evaporated at different rates, and Compound HI-1 was deposited in a doping amount of 5 wt % based on the total amount of Compounds HI-1 and HT-3 to form a hole injection layer having a thickness of 10 nm. Next, Compound HT-3 was deposited as a first hole transport layer having a thickness of 80 nm on the hole injection layer. Compound HT-4 was then introduced into another cell of the vacuum vapor deposition apparatus and was evaporated by applying an electric current to the cell, thereby forming a second hole transport layer having a thickness of 15 nm on the first hole transport layer. After formation of the hole injection layer and the hole transport layers, a first light-emitting layer was formed thereon as follows: The compound described in Table 7 below was introduced into a cell of the vacuum vapor deposition apparatus as a host, and Compound D-2 was introduced into another cell as a dopant. The two materials were evaporated at different rates and co-deposited with 2 wt % of the dopant, based on the total amount of the host and dopant, to form a 5 nm-thick first light-emitting layer on the second hole transport layer. A second light-emitting layer was then formed on the first light-emitting layer as follows: Compound H2-1 and Compound H2-2 were introduced into two cells of the vacuum vapor deposition apparatus as hosts, respectively, and Compound D-2 was introduced into another cell as a dopant. The host materials were evaporated at the same rate, and the dopant was evaporated at a different rate and was deposited in a doping amount of 2 wt % based on the total amount of the hosts and dopant to form a second light-emitting layer having a thickness of 13 nm on the first light-emitting layer. After deposition of the light-emitting layers, Compound ET-1 was deposited as a hole-blocking layer material to form a hole-blocking layer with a thickness of 5 nm. Compound ET-3 and Compound EI-1 were then introduced into two cells of the vacuum vapor deposition apparatus as electron transport layer materials, respectively. And then the two materials were evaporated at a ratio of 2:1 to deposit an electron transport layer having a thickness of 25 nm. Thereafter, Yb and LiF were deposited as an electron injection layer at a ratio of 2:1 to form an electron injection layer having a thickness of 1 nm on the electron transport layer. An Al cathode having a thickness of 80 nm was then deposited on the electron injection layer by another vacuum vapor deposition apparatus. Thus, OLEDs were produced. Each of the compounds used for all of the materials were purified by vacuum sublimation at 10⁻⁶ Torr.

### [Device Comparative Example 4] Preparation of an OLED Comprising a Single Light-Emitting Layer

An OLED was manufactured in the same manner as Device Example 8, except that instead of the first light-emitting layer and the second light-emitting layer, the compound of Table 7 below was used as a host material in the same ratio, and Compound D-2 was deposited as a dopant in a doping amount of 2 wt % based on the total amount of the host and dopant, thereby forming a single light-emitting layer having a thickness of 18 nm.

The driving voltage at a luminance of 1,000 nits, the luminous efficacy, and the time taken for the light intensity to decrease from 100% to 95% when checking the lifespan with 2× acceleration (lifespan; T₉₅) of the OLEDs of Device Examples 8 to 17 and Device Comparative Example 4 produced as described above were measured, and the results thereof are shown in Table 7 below.

TABLE 7

| | First Light- | Second Light- | Driving | Current | |
|---|---|---|---|---|---|
| | Emitting | Emitting | Voltage | Efficiency | Lifespan |
| | Layer Host | Layer Host | [V] | [cd/A] | T₉₅ [hr] |
| Device Example | C-5 | H2-1:H2-2 | 3.5 | 6.0 | 103.0 |
| 8 | | | | | |
| Device Example | C-1140 | H2-1:H2-2 | 3.5 | 6.4 | 125.9 |
| 9 | | | | | |
| Device Example | C-79 | H2-1:H2-2 | 3.3 | 12.2 | 52.7 |
| 10 | | | | | |
| Device Example | C-111 | H2-1:H2-2 | 3.5 | 6.3 | 199.1 |
| 11 | | | | | |
| Device Example | C-2 | H2-1:H2-2 | 3.4 | 6.5 | 113.9 |
| 12 | | | | | |
| Device Example | C-221 | H2-1:H2-2 | 3.5 | 6.1 | 100.8 |
| 13 | | | | | |
| Device Example | C-301 | H2-1:H2-2 | 3.6 | 5.9 | 100.8 |
| 14 | | | | | |
| Device Example | C-222 | H2-1:H2-2 | 3.5 | 6.6 | 150.7 |
| 15 | | | | | |
| Device Example | C-167 | H2-1:H2-2 | 3.5 | 6.4 | 147.5 |
| 16 | | | | | |
| Device Example | C-170 | H2-1:H2-2 | 3.5 | 6.2 | 117.8 |
| 17 | | | | | |
| Device | — | H2-1:H2-2 | 3.5 | 5.6 | 95.0 |
| Comparative | | | | | |
| Example 4 | | | | | |

Referring to Table 7 above, it can be confirmed that an organic electroluminescent device including an organic electroluminescent compound according to the present disclosure in each of a plurality of light-emitting layers according to the present disclosure has lower driving voltage and/or higher current efficiency and/or longer lifespan characteristics compared to an organic electroluminescent device including only a single light-emitting layer.

The compounds used in Device Examples 8 to 17 and Device Comparative Example 4 are specifically shown in Table 8 below.

TABLE 8

| | |
|---|---|
| Hole Injection Layer/Hole Transport Layer | 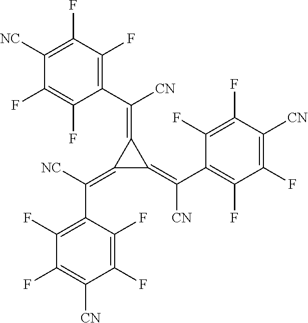 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 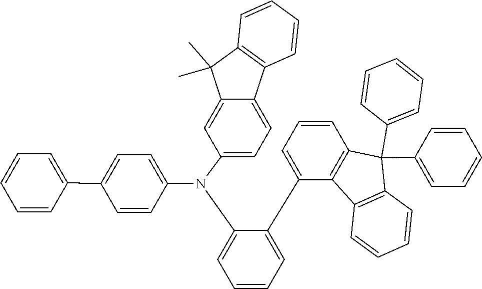 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-3 |
| | 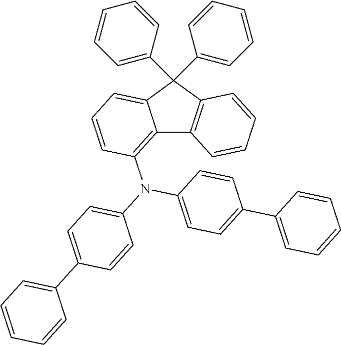 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-4 |
| Light-Emitting Layer | 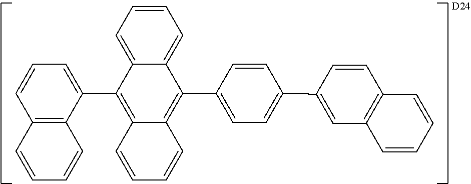 |
| | H2-1 |
| | 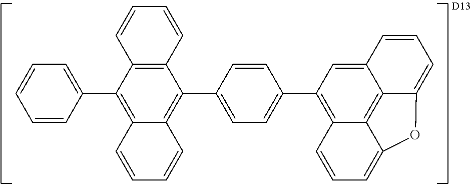 |
| | H2-2 |
| | 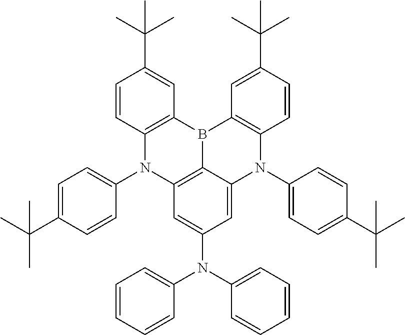 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | D-2 |
| | 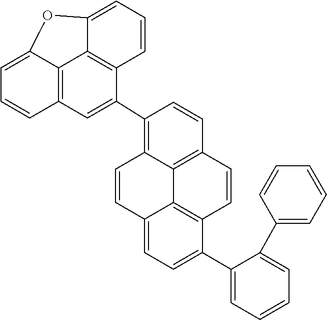 [SMILES: c1ccc(-c2ccccc2-c2ccc3ccc4c(-c5cc6cccc7oc8cccc5c8c67)ccc5ccc2c3c54)cc1] |
| | C-5 |
| | 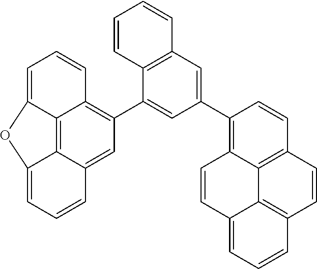 [SMILES: c1ccc2c(-c3cc4cccc5oc6cccc3c6c45)cc(-c3ccc4ccc5cccc6ccc3c4c56)cc2c1] |
| | C-1140 |
| | 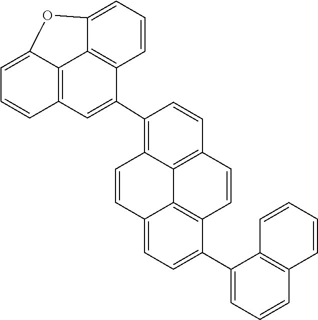 [SMILES: c1ccc2c(-c3ccc4ccc5c(-c6cc7cccc8oc9cccc6c9c78)ccc6ccc3c4c65)cccc2c1] |
| | C-79 |
| | 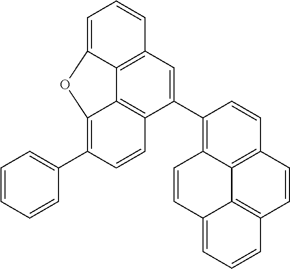 [SMILES: c1ccc(-c2ccc3c(-c4ccc5ccc6cccc7ccc4c5c67)cc4cccc5oc2c3c45)cc1] |
| | C-111 |
| | 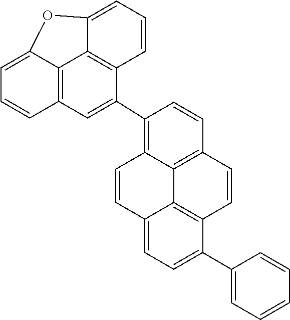 [SMILES: c1ccc(-c2ccc3ccc4c(-c5cc6cccc7oc8cccc5c8c67)ccc5ccc2c3c54)cc1] |
| | C-2 |
| | 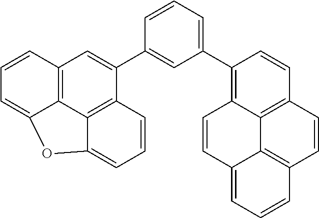 [SMILES: c1cc(-c2ccc3ccc4cccc5ccc2c3c45)cc(-c2cc3cccc4oc5cccc2c5c34)c1] |
| | C-221 |
| | 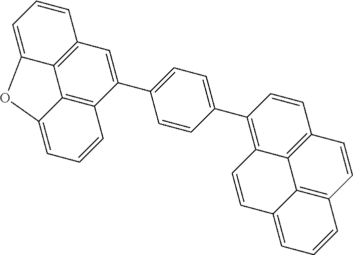 [SMILES: c1cc2ccc3ccc(-c4ccc(-c5cc6cccc7oc8cccc5c8c67)cc4)c4ccc(c1)c2c34] |
| | C-301 |
| | 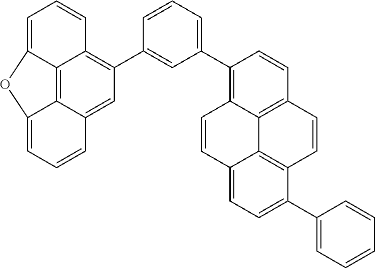 [SMILES: c1ccc(-c2ccc3ccc4c(-c5cccc(-c6cc7cccc8oc9cccc6c9c78)c5)ccc5ccc2c3c54)cc1] |
| | C-222 |
| | 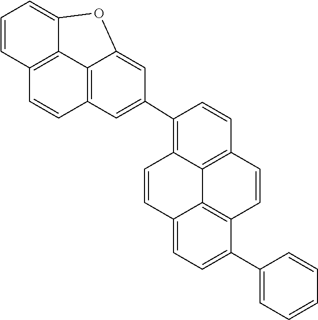 [SMILES: c1ccc(-c2ccc3ccc4c(-c5cc6ccc7cccc8oc(c5)c6c78)ccc5ccc2c3c54)cc1] |
| | C-167 |
| | 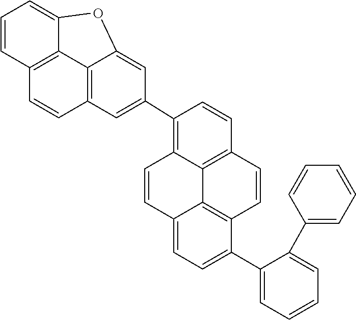 [SMILES: c1ccc(-c2ccccc2-c2ccc3ccc4c(-c5cc6ccc7cccc8oc(c5)c6c78)ccc5ccc2c3c54)cc1] |
| | C-170 |
| Hole-Blocking Layer/ Electron Transport Layer/ Electron Injection Layer | 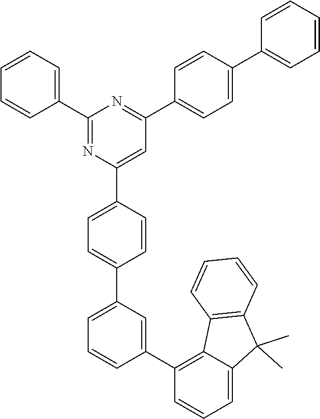 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 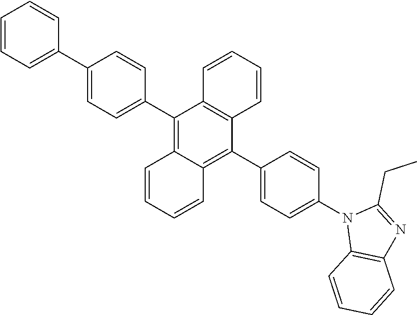 [SMILES: CCc1nc2ccccc2n1-c1ccc(-c2c3ccccc3c(-c3ccc(-c4ccccc4)cc3)c3ccccc23)cc1] |
| | ET-3 |
| | 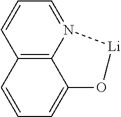 |
| | EI-1 |

1. An organic electroluminescent compound represented by the following Formula 1: 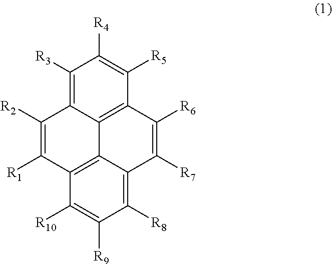
wherein,
R₁ to R₁₀ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s);
provided that at least one of R₁ to R₁₀ is -L-Ar;
L represents a single bond, a substituted or unsubstituted (C₆-C₃₀)arylene, or a substituted or unsubstituted (3- to 30-membered)heteroarylene; and
Ar is represented by the following Formula 1A: 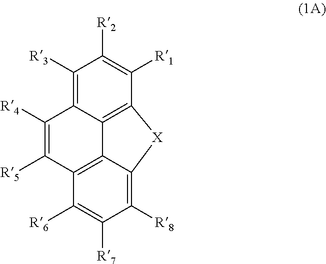
wherein,
X represents —O—, —NR′₉, or —CR′₁₀R′₁₁;
any one of R′₁ to R′₈ is a site linked to the above L, and R′₁ to R′₈ which do not link to the above L each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s); and
R′₉ to R′₁₁ each independently represent a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or R′₁₀ and R′₁₁ may be linked to each other to form a ring(s).

2. The organic electroluminescent compound according to claim 1, wherein at least one of R₃ to R₅ is -L-Ar.

3. The organic electroluminescent compound according to claim 1, wherein the organic electroluminescent compound represented by Formula 1 comprises at least one deuterium.

4. The organic electroluminescent compound according to claim 1, wherein the organic electroluminescent compound represented by Formula 1 is represented by the following Formula 1-1: 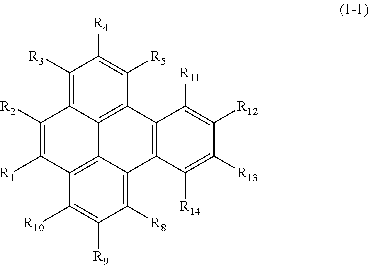
wherein,
R₁ to R₄ and R₈ to R₁₀ are as defined in claim 1; and
R₁₁ to R₁₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring; or may be linked to the adjacent substituents to form a ring(s).

5. The organic electroluminescent compound according to claim 1, wherein the organic electroluminescent compound represented by Formula 1 is selected from the following compounds: 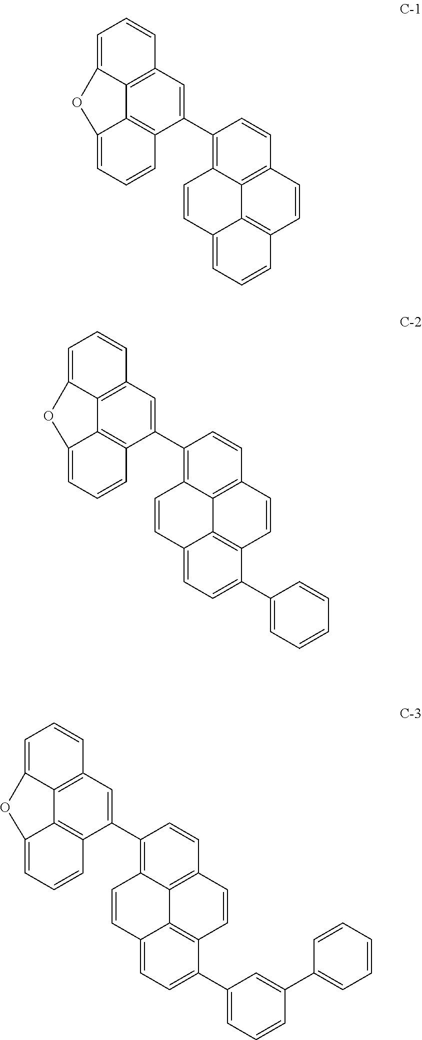 [SMILES: c1cc2ccc3ccc(-c4cc5cccc6oc7cccc4c7c56)c4ccc(c1)c2c34 ; c1ccc(-c2ccc3ccc4c(-c5cc6cccc7oc8cccc5c8c67)ccc5ccc2c3c54)cc1 ; c1ccc(-c2cccc(-c3ccc4ccc5c(-c6cc7cccc8oc9cccc6c9c78)ccc6ccc3c4c65)c2)cc1] 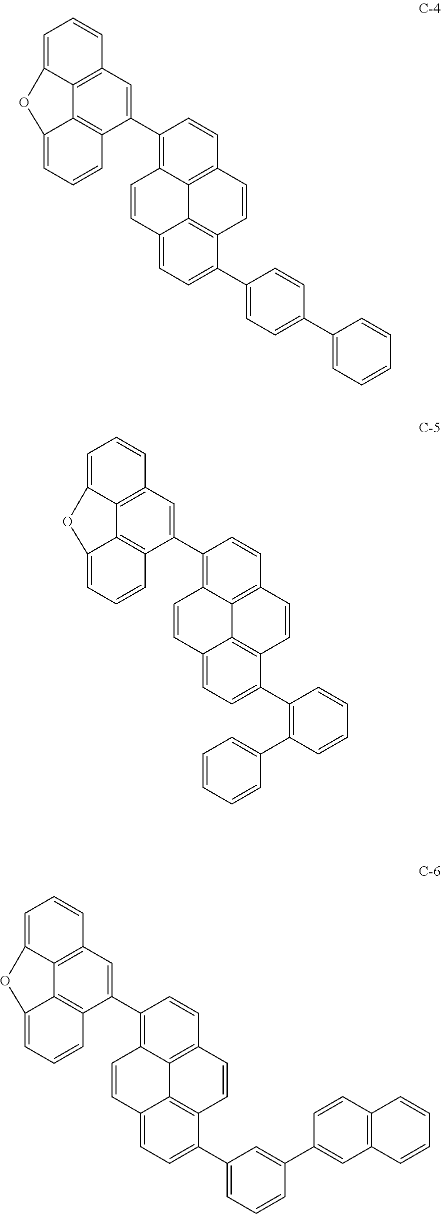 [SMILES: c1ccc(-c2ccc(-c3ccc4ccc5c(-c6cc7cccc8oc9cccc6c9c78)ccc6ccc3c4c65)cc2)cc1 ; c1ccc(-c2ccccc2-c2ccc3ccc4c(-c5cc6cccc7oc8cccc5c8c67)ccc5ccc2c3c54)cc1 ; c1cc(-c2ccc3ccccc3c2)cc(-c2ccc3ccc4c(-c5cc6cccc7oc8cccc5c8c67)ccc5ccc2c3c54)c1] 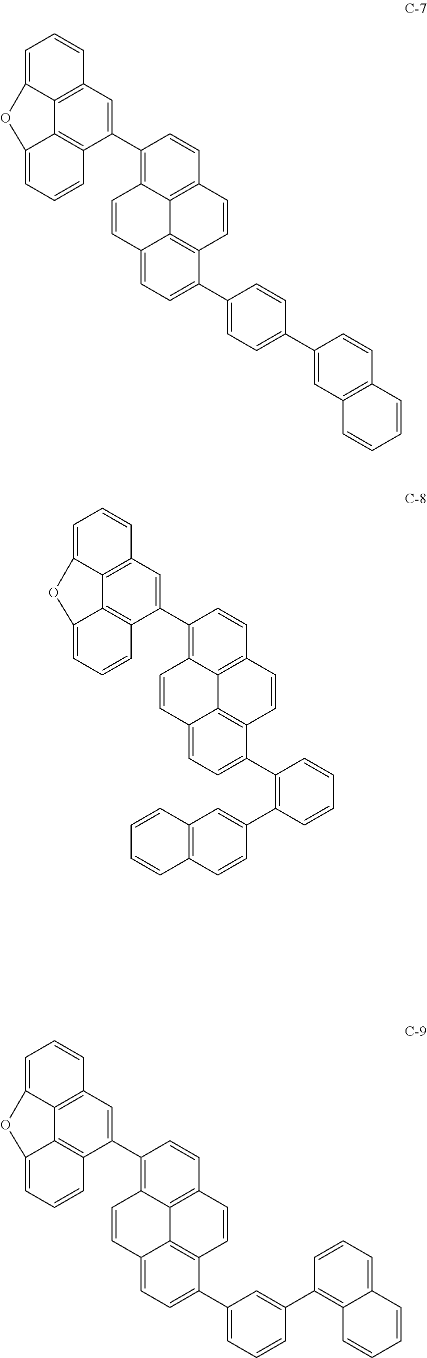 [SMILES: c1ccc2cc(-c3ccc(-c4ccc5ccc6c(-c7cc8cccc9oc%10cccc7c%10c89)ccc7ccc4c5c76)cc3)ccc2c1 ; c1ccc(-c2ccc3ccc4c(-c5cc6cccc7oc8cccc5c8c67)ccc5ccc2c3c54)c(-c2ccc3ccccc3c2)c1 ; c1cc(-c2cccc3ccccc23)cc(-c2ccc3ccc4c(-c5cc6cccc7oc8cccc5c8c67)ccc5ccc2c3c54)c1] 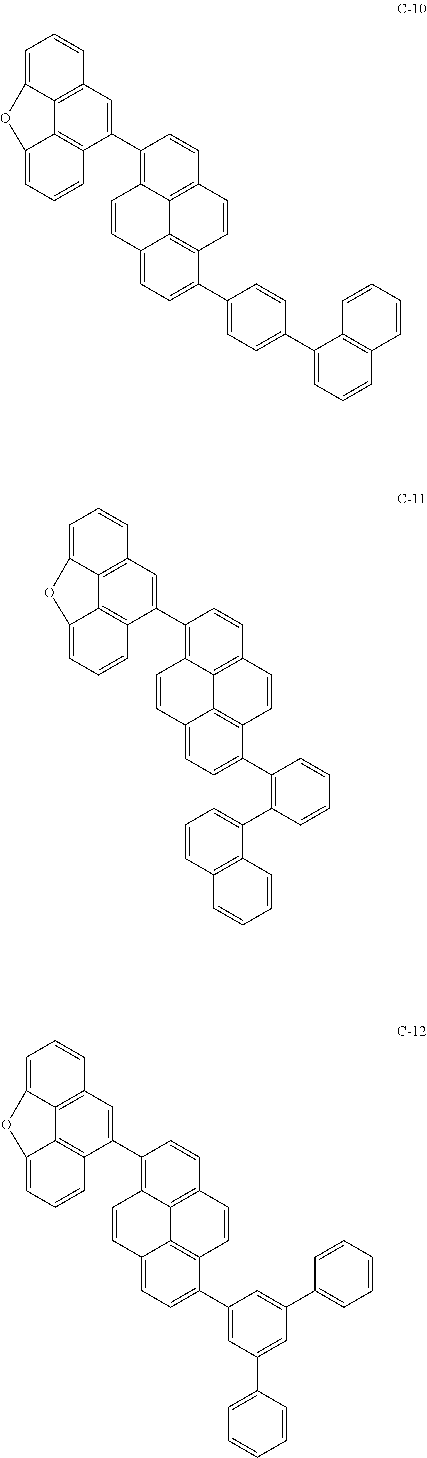 [SMILES: c1ccc2c(-c3ccc(-c4ccc5ccc6c(-c7cc8cccc9oc%10cccc7c%10c89)ccc7ccc4c5c76)cc3)cccc2c1 ; c1ccc(-c2ccc3ccc4c(-c5cc6cccc7oc8cccc5c8c67)ccc5ccc2c3c54)c(-c2cccc3ccccc23)c1 ; c1ccc(-c2cc(-c3ccccc3)cc(-c3ccc4ccc5c(-c6cc7cccc8oc9cccc6c9c78)ccc6ccc3c4c65)c2)cc1] 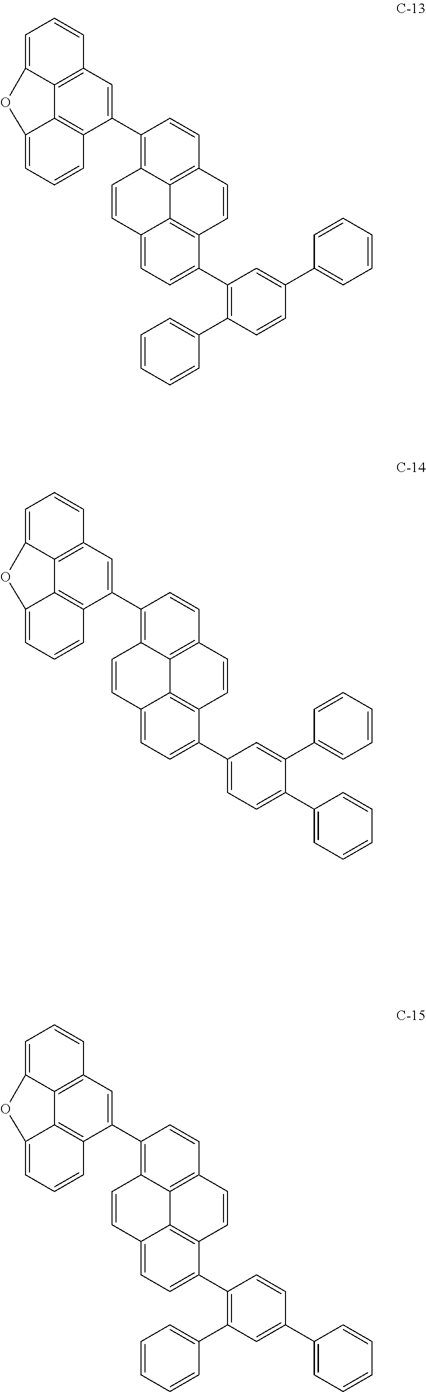 [SMILES: c1ccc(-c2ccc(-c3ccccc3)c(-c3ccc4ccc5c(-c6cc7cccc8oc9cccc6c9c78)ccc6ccc3c4c65)c2)cc1 ; c1ccc(-c2ccc(-c3ccc4ccc5c(-c6cc7cccc8oc9cccc6c9c78)ccc6ccc3c4c65)cc2-c2ccccc2)cc1 ; c1ccc(-c2ccc(-c3ccc4ccc5c(-c6cc7cccc8oc9cccc6c9c78)ccc6ccc3c4c65)c(-c3ccccc3)c2)cc1] 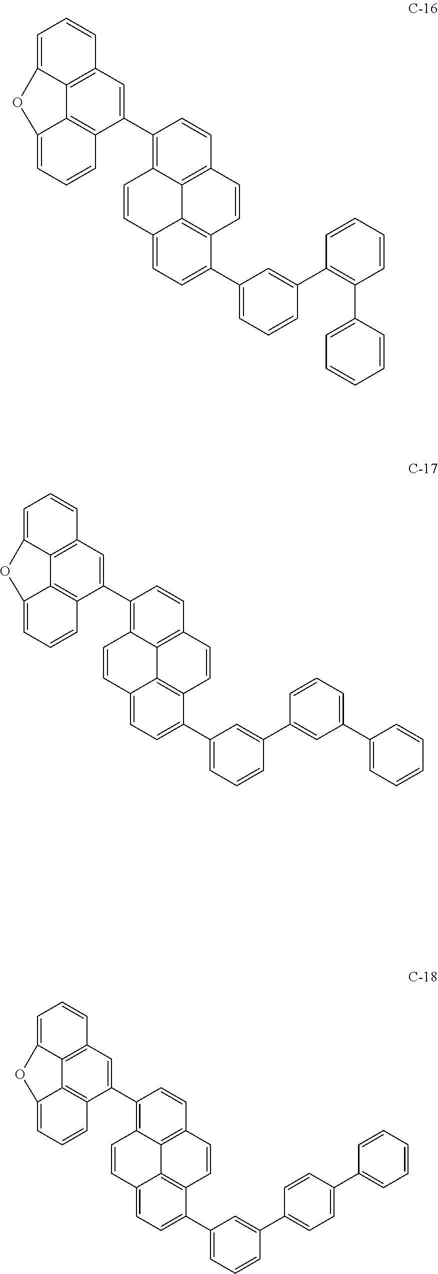 [SMILES: c1ccc(-c2ccccc2-c2cccc(-c3ccc4ccc5c(-c6cc7cccc8oc9cccc6c9c78)ccc6ccc3c4c65)c2)cc1 ; c1ccc(-c2cccc(-c3cccc(-c4ccc5ccc6c(-c7cc8cccc9oc%10cccc7c%10c89)ccc7ccc4c5c76)c3)c2)cc1 ; c1ccc(-c2ccc(-c3cccc(-c4ccc5ccc6c(-c7cc8cccc9oc%10cccc7c%10c89)ccc7ccc4c5c76)c3)cc2)cc1] 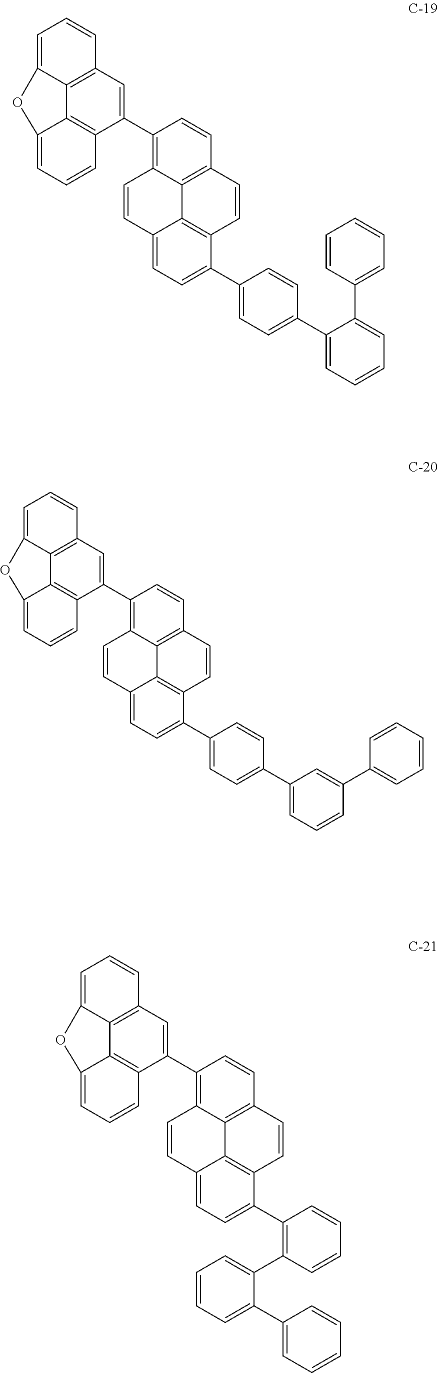 [SMILES: c1ccc(-c2ccccc2-c2ccc(-c3ccc4ccc5c(-c6cc7cccc8oc9cccc6c9c78)ccc6ccc3c4c65)cc2)cc1 ; c1ccc(-c2cccc(-c3ccc(-c4ccc5ccc6c(-c7cc8cccc9oc%10cccc7c%10c89)ccc7ccc4c5c76)cc3)c2)cc1 ; c1ccc(-c2ccccc2-c2ccccc2-c2ccc3ccc4c(-c5cc6cccc7oc8cccc5c8c67)ccc5ccc2c3c54)cc1] 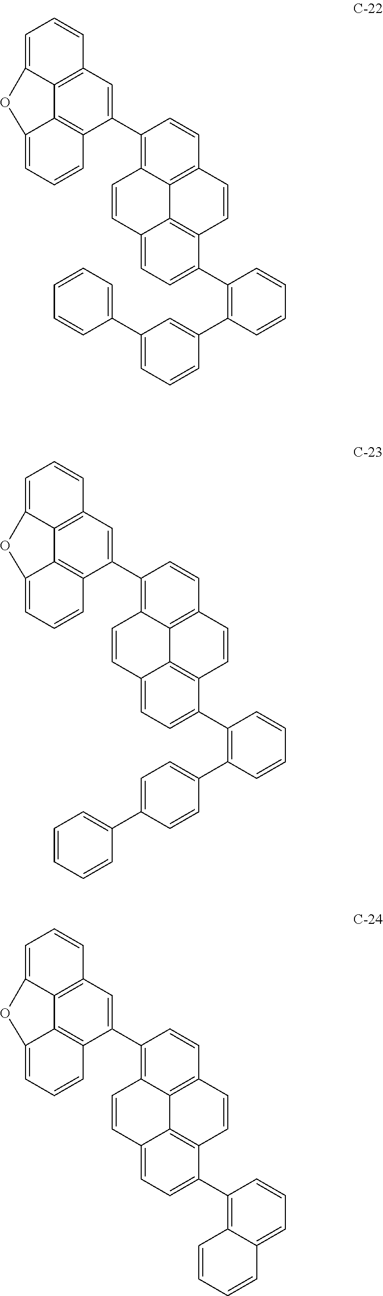 [SMILES: c1ccc(-c2cccc(-c3ccccc3-c3ccc4ccc5c(-c6cc7cccc8oc9cccc6c9c78)ccc6ccc3c4c65)c2)cc1 ; c1ccc(-c2ccc(-c3ccccc3-c3ccc4ccc5c(-c6cc7cccc8oc9cccc6c9c78)ccc6ccc3c4c65)cc2)cc1 ; c1ccc2c(-c3ccc4ccc5c(-c6cc7cccc8oc9cccc6c9c78)ccc6ccc3c4c65)cccc2c1] 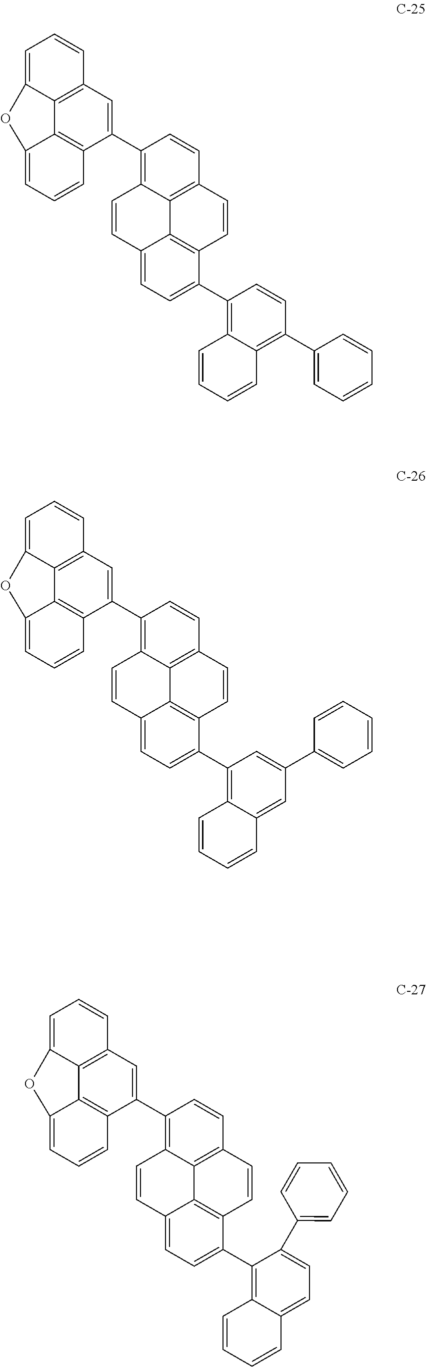 [SMILES: c1ccc(-c2ccc(-c3ccc4ccc5c(-c6cc7cccc8oc9cccc6c9c78)ccc6ccc3c4c65)c3ccccc23)cc1 ; c1ccc(-c2cc(-c3ccc4ccc5c(-c6cc7cccc8oc9cccc6c9c78)ccc6ccc3c4c65)c3ccccc3c2)cc1 ; c1ccc(-c2ccc3ccccc3c2-c2ccc3ccc4c(-c5cc6cccc7oc8cccc5c8c67)ccc5ccc2c3c54)cc1] 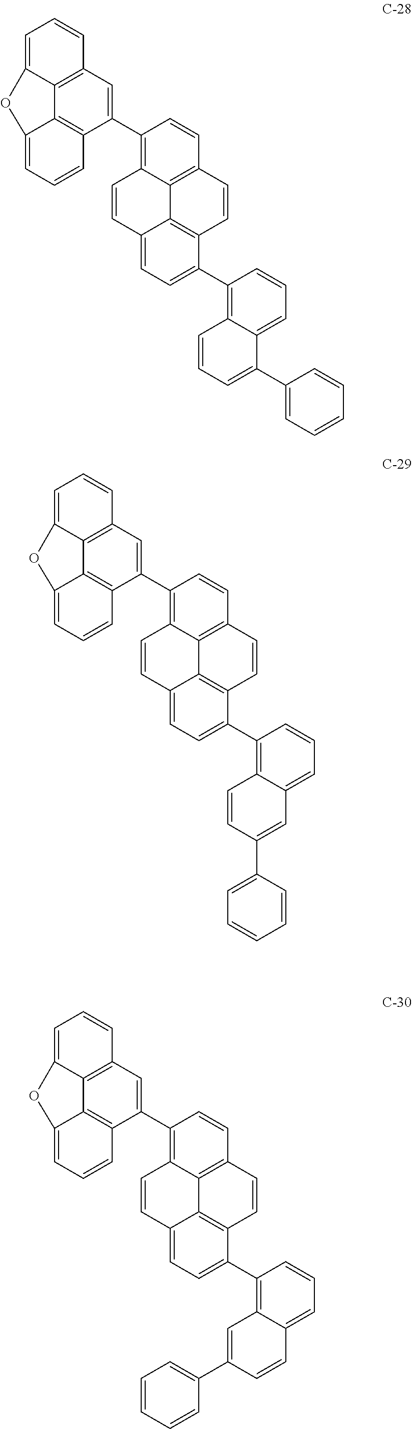 [SMILES: c1ccc(-c2cccc3c(-c4ccc5ccc6c(-c7cc8cccc9oc%10cccc7c%10c89)ccc7ccc4c5c76)cccc23)cc1 ; c1ccc(-c2ccc3c(-c4ccc5ccc6c(-c7cc8cccc9oc%10cccc7c%10c89)ccc7ccc4c5c76)cccc3c2)cc1 ; c1ccc(-c2ccc3cccc(-c4ccc5ccc6c(-c7cc8cccc9oc%10cccc7c%10c89)ccc7ccc4c5c76)c3c2)cc1] 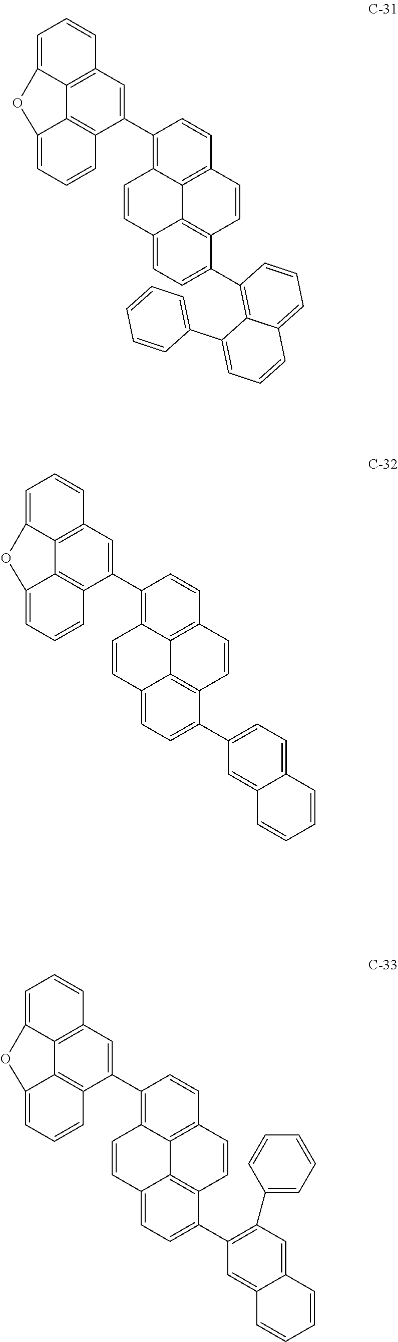 [SMILES: c1ccc(-c2cccc3cccc(-c4ccc5ccc6c(-c7cc8cccc9oc%10cccc7c%10c89)ccc7ccc4c5c76)c23)cc1 ; c1ccc2cc(-c3ccc4ccc5c(-c6cc7cccc8oc9cccc6c9c78)ccc6ccc3c4c65)ccc2c1 ; c1ccc(-c2cc3ccccc3cc2-c2ccc3ccc4c(-c5cc6cccc7oc8cccc5c8c67)ccc5ccc2c3c54)cc1] 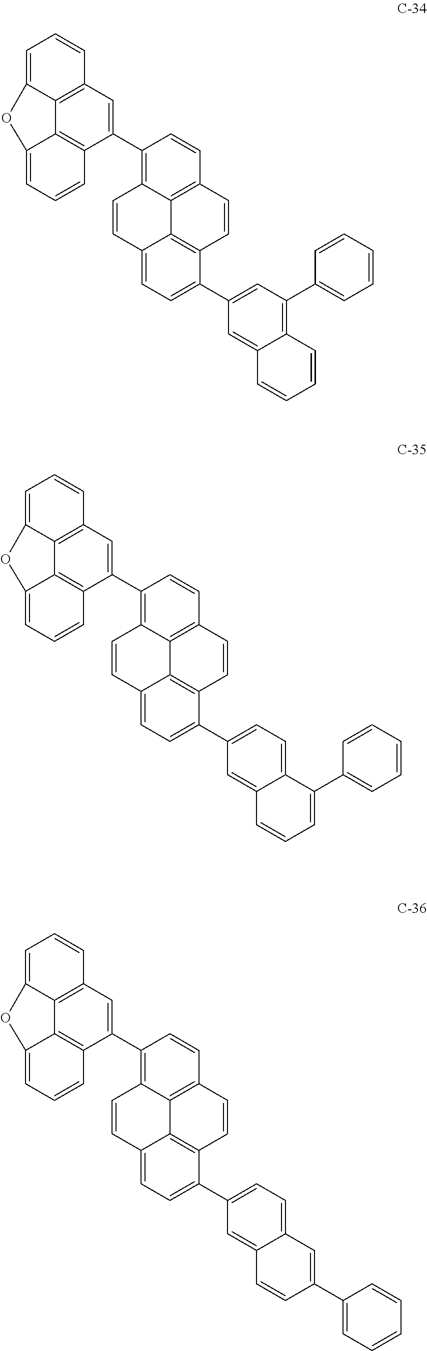 [SMILES: c1ccc(-c2cc(-c3ccc4ccc5c(-c6cc7cccc8oc9cccc6c9c78)ccc6ccc3c4c65)cc3ccccc23)cc1 ; c1ccc(-c2cccc3cc(-c4ccc5ccc6c(-c7cc8cccc9oc%10cccc7c%10c89)ccc7ccc4c5c76)ccc23)cc1 ; c1ccc(-c2ccc3cc(-c4ccc5ccc6c(-c7cc8cccc9oc%10cccc7c%10c89)ccc7ccc4c5c76)ccc3c2)cc1] 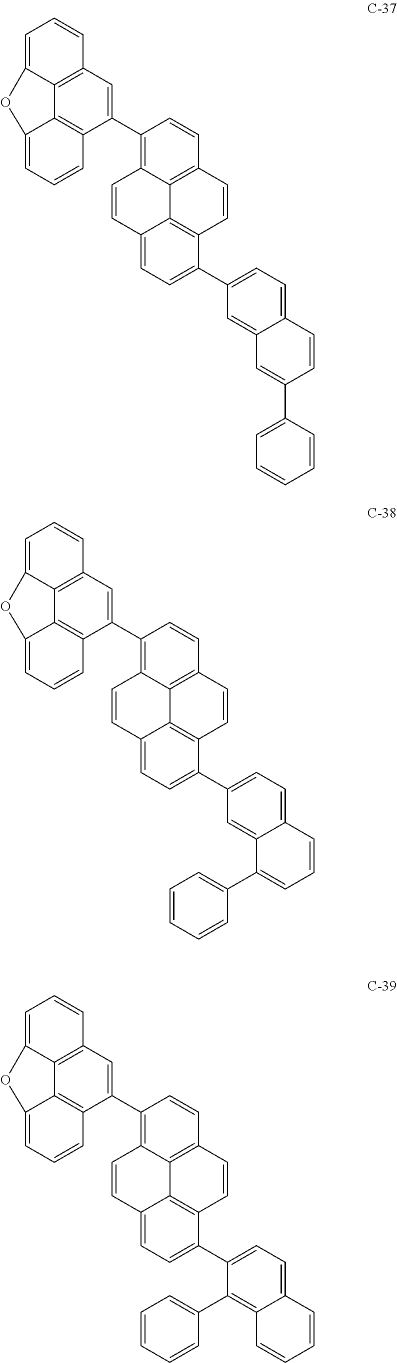 [SMILES: c1ccc(-c2ccc3ccc(-c4ccc5ccc6c(-c7cc8cccc9oc%10cccc7c%10c89)ccc7ccc4c5c76)cc3c2)cc1 ; c1ccc(-c2cccc3ccc(-c4ccc5ccc6c(-c7cc8cccc9oc%10cccc7c%10c89)ccc7ccc4c5c76)cc23)cc1 ; c1ccc(-c2c(-c3ccc4ccc5c(-c6cc7cccc8oc9cccc6c9c78)ccc6ccc3c4c65)ccc3ccccc23)cc1] 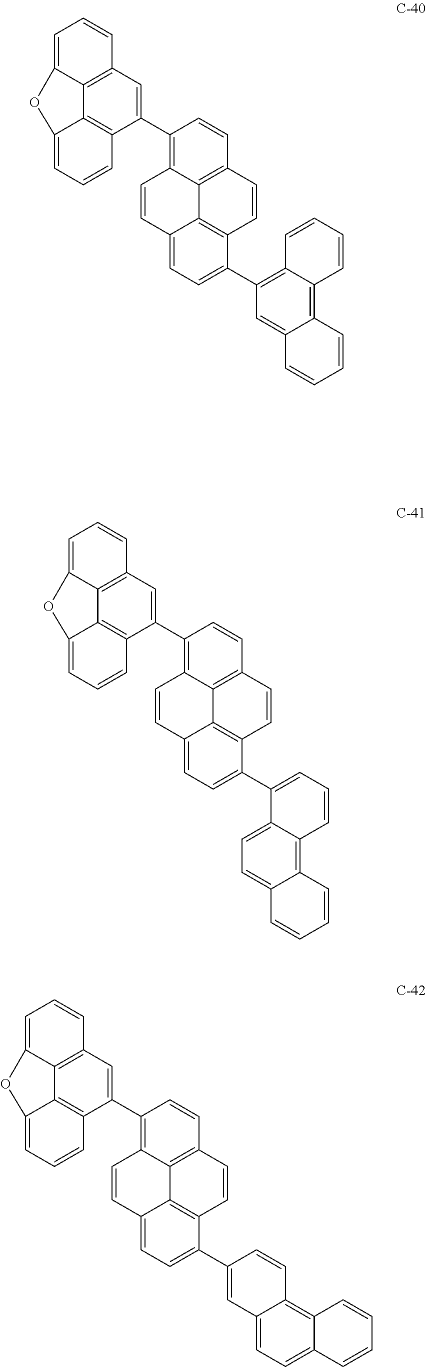 [SMILES: c1ccc2c(c1)cc(-c1ccc3ccc4c(-c5cc6cccc7oc8cccc5c8c67)ccc5ccc1c3c54)c1ccccc12 ; c1ccc2c(c1)ccc1c(-c3ccc4ccc5c(-c6cc7cccc8oc9cccc6c9c78)ccc6ccc3c4c65)cccc12 ; c1ccc2c(c1)ccc1cc(-c3ccc4ccc5c(-c6cc7cccc8oc9cccc6c9c78)ccc6ccc3c4c65)ccc12] 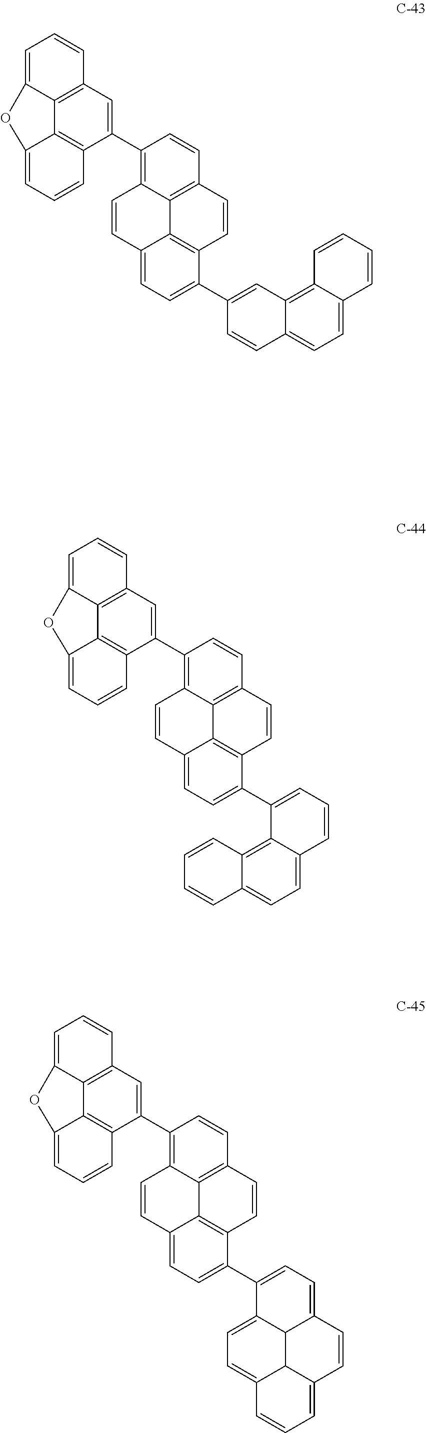 [SMILES: c1ccc2c(c1)ccc1ccc(-c3ccc4ccc5c(-c6cc7cccc8oc9cccc6c9c78)ccc6ccc3c4c65)cc12 ; c1ccc2c(c1)ccc1cccc(-c3ccc4ccc5c(-c6cc7cccc8oc9cccc6c9c78)ccc6ccc3c4c65)c12 ; C1=CC2=CC=C3C(c4ccc5ccc6c(-c7cc8cccc9oc%10cccc7c%10c89)ccc7ccc4c5c76)=CC=C4C=CC(=C1)C2C43] 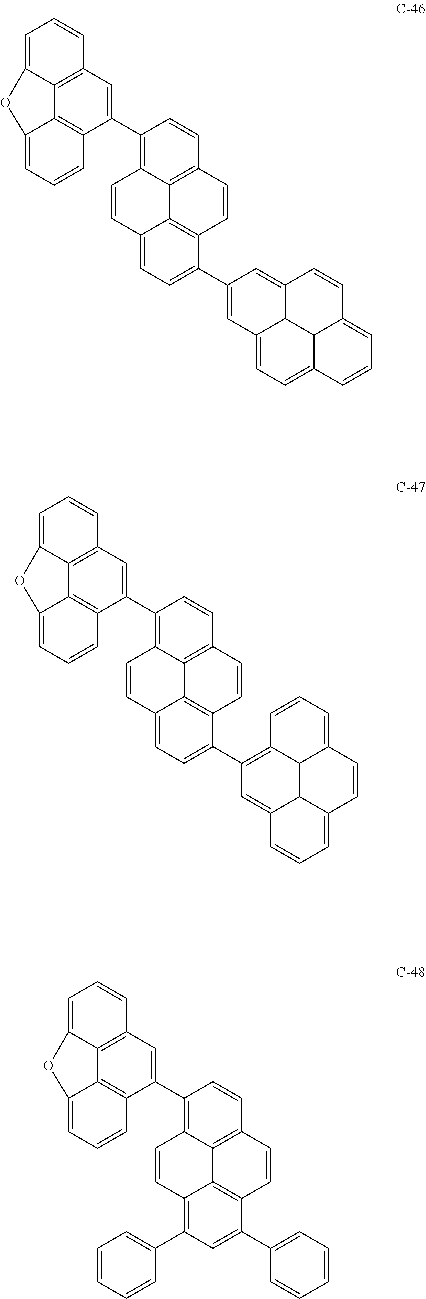 [SMILES: C1=CC2=CC=C3C=C(c4ccc5ccc6c(-c7cc8cccc9oc%10cccc7c%10c89)ccc7ccc4c5c76)C=C4C=CC(=C1)C2C34 ; C1=CC2=CC(c3ccc4ccc5c(-c6cc7cccc8oc9cccc6c9c78)ccc6ccc3c4c65)=C3C=CC=C4C=CC(=C1)C2C43 ; c1ccc(-c2cc(-c3ccccc3)c3ccc4c(-c5cc6cccc7oc8cccc5c8c67)ccc5ccc2c3c54)cc1] 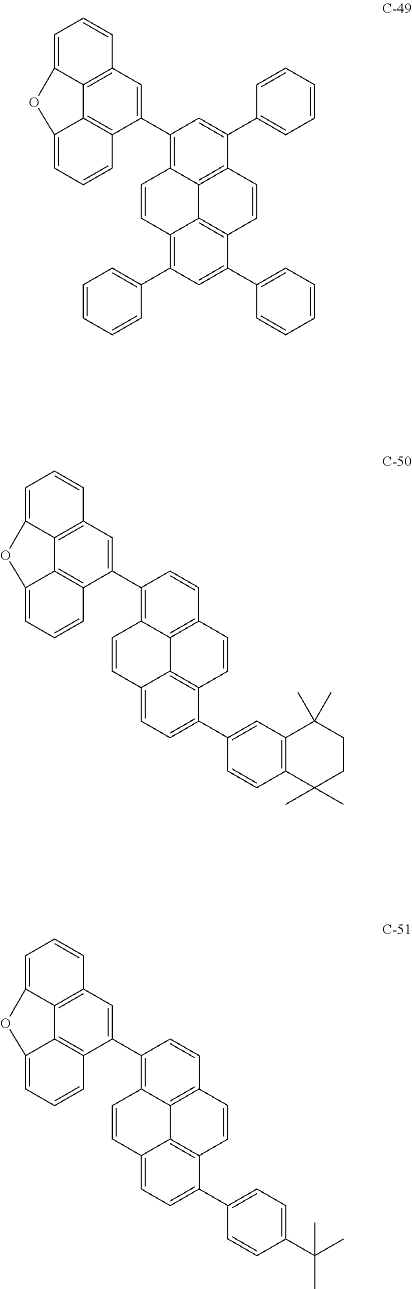 [SMILES: c1ccc(-c2cc(-c3ccccc3)c3ccc4c(-c5cc6cccc7oc8cccc5c8c67)cc(-c5ccccc5)c5ccc2c3c54)cc1 ; CC1(C)CCC(C)(C)c2cc(-c3ccc4ccc5c(-c6cc7cccc8oc9cccc6c9c78)ccc6ccc3c4c65)ccc21 ; CC(C)(C)c1ccc(-c2ccc3ccc4c(-c5cc6cccc7oc8cccc5c8c67)ccc5ccc2c3c54)cc1] 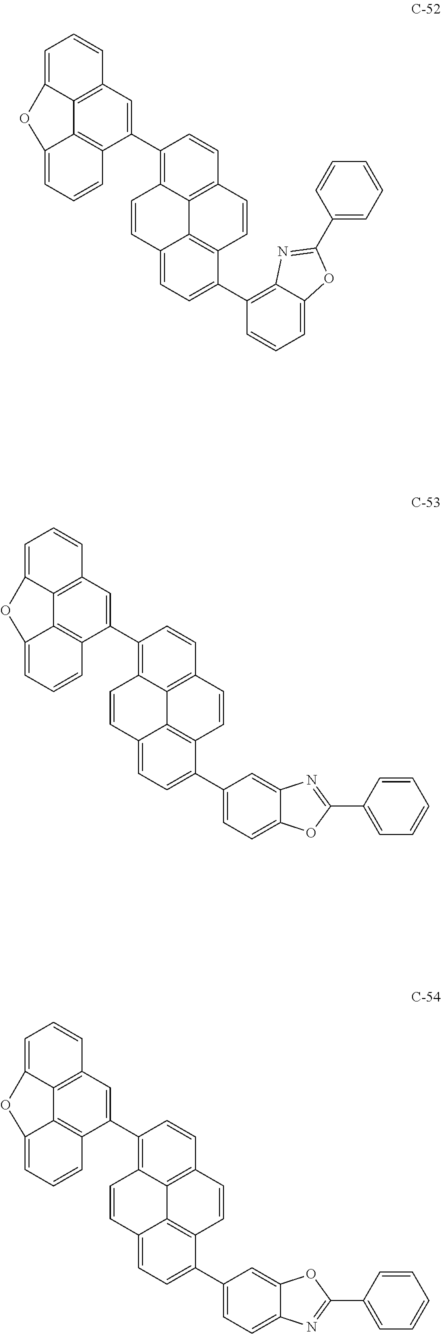 [SMILES: c1ccc(-c2nc3c(-c4ccc5ccc6c(-c7cc8cccc9oc%10cccc7c%10c89)ccc7ccc4c5c76)cccc3o2)cc1 ; c1ccc(-c2nc3cc(-c4ccc5ccc6c(-c7cc8cccc9oc%10cccc7c%10c89)ccc7ccc4c5c76)ccc3o2)cc1 ; c1ccc(-c2nc3ccc(-c4ccc5ccc6c(-c7cc8cccc9oc%10cccc7c%10c89)ccc7ccc4c5c76)cc3o2)cc1] 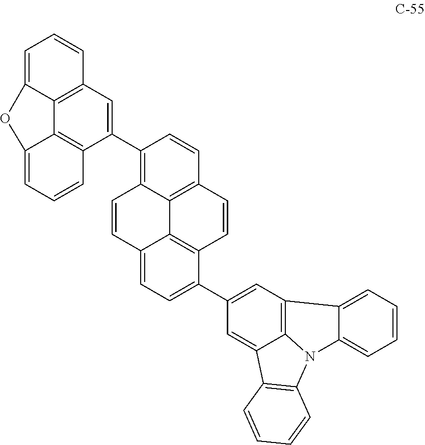 [SMILES: c1cc2cc(-c3ccc4ccc5c(-c6cc7c8ccccc8n8c9ccccc9c(c6)c78)ccc6ccc3c4c65)c3cccc4oc(c1)c2c43] 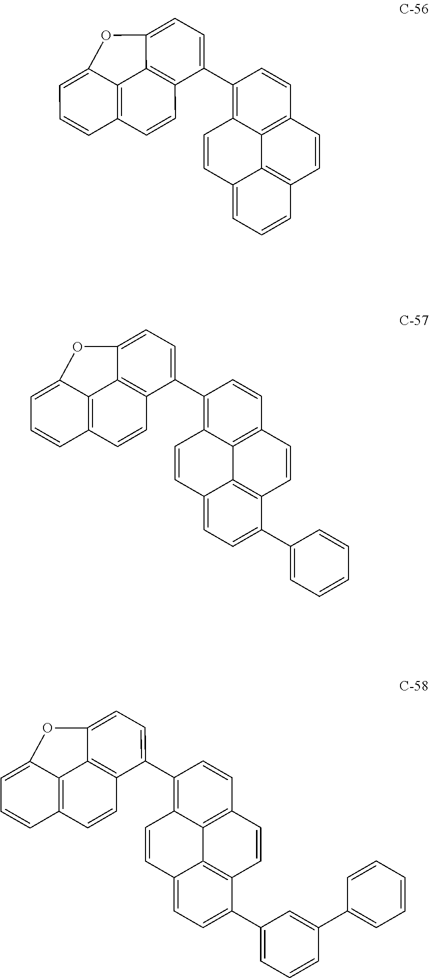 [SMILES: c1cc2ccc3ccc(-c4ccc5oc6cccc7ccc4c5c76)c4ccc(c1)c2c34 ; c1ccc(-c2ccc3ccc4c(-c5ccc6oc7cccc8ccc5c6c87)ccc5ccc2c3c54)cc1 ; c1ccc(-c2cccc(-c3ccc4ccc5c(-c6ccc7oc8cccc9ccc6c7c98)ccc6ccc3c4c65)c2)cc1] 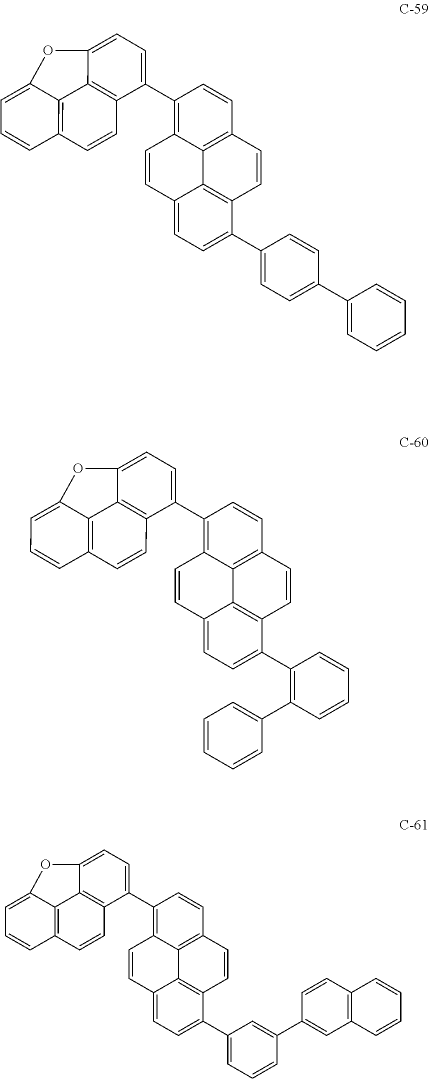 [SMILES: c1ccc(-c2ccc(-c3ccc4ccc5c(-c6ccc7oc8cccc9ccc6c7c98)ccc6ccc3c4c65)cc2)cc1 ; c1ccc(-c2ccccc2-c2ccc3ccc4c(-c5ccc6oc7cccc8ccc5c6c87)ccc5ccc2c3c54)cc1 ; c1cc(-c2ccc3ccccc3c2)cc(-c2ccc3ccc4c(-c5ccc6oc7cccc8ccc5c6c87)ccc5ccc2c3c54)c1] 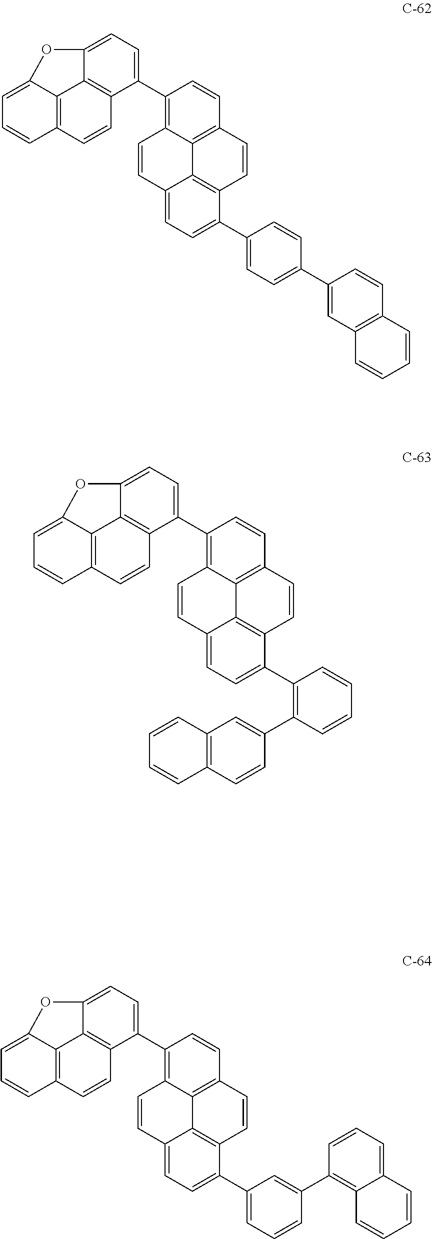 [SMILES: c1ccc2cc(-c3ccc(-c4ccc5ccc6c(-c7ccc8oc9cccc%10ccc7c8c%109)ccc7ccc4c5c76)cc3)ccc2c1 ; c1ccc(-c2ccc3ccc4c(-c5ccc6oc7cccc8ccc5c6c87)ccc5ccc2c3c54)c(-c2ccc3ccccc3c2)c1 ; c1cc(-c2cccc3ccccc23)cc(-c2ccc3ccc4c(-c5ccc6oc7cccc8ccc5c6c87)ccc5ccc2c3c54)c1] 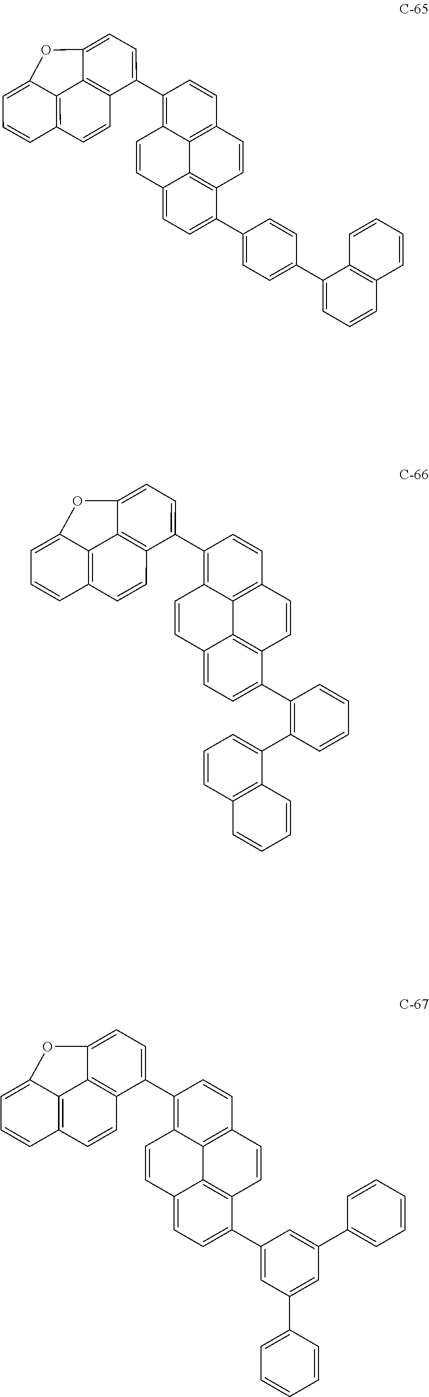 [SMILES: c1ccc2c(-c3ccc(-c4ccc5ccc6c(-c7ccc8oc9cccc%10ccc7c8c%109)ccc7ccc4c5c76)cc3)cccc2c1 ; c1ccc(-c2ccc3ccc4c(-c5ccc6oc7cccc8ccc5c6c87)ccc5ccc2c3c54)c(-c2cccc3ccccc23)c1 ; c1ccc(-c2cc(-c3ccccc3)cc(-c3ccc4ccc5c(-c6ccc7oc8cccc9ccc6c7c98)ccc6ccc3c4c65)c2)cc1] 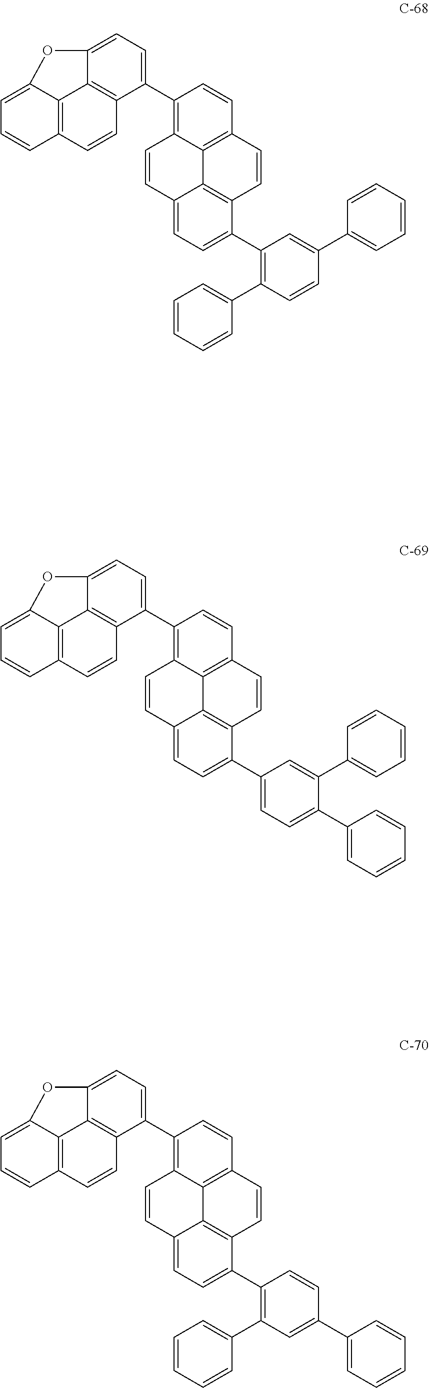 [SMILES: c1ccc(-c2ccc(-c3ccccc3)c(-c3ccc4ccc5c(-c6ccc7oc8cccc9ccc6c7c98)ccc6ccc3c4c65)c2)cc1 ; c1ccc(-c2ccc(-c3ccc4ccc5c(-c6ccc7oc8cccc9ccc6c7c98)ccc6ccc3c4c65)cc2-c2ccccc2)cc1 ; c1ccc(-c2ccc(-c3ccc4ccc5c(-c6ccc7oc8cccc9ccc6c7c98)ccc6ccc3c4c65)c(-c3ccccc3)c2)cc1] 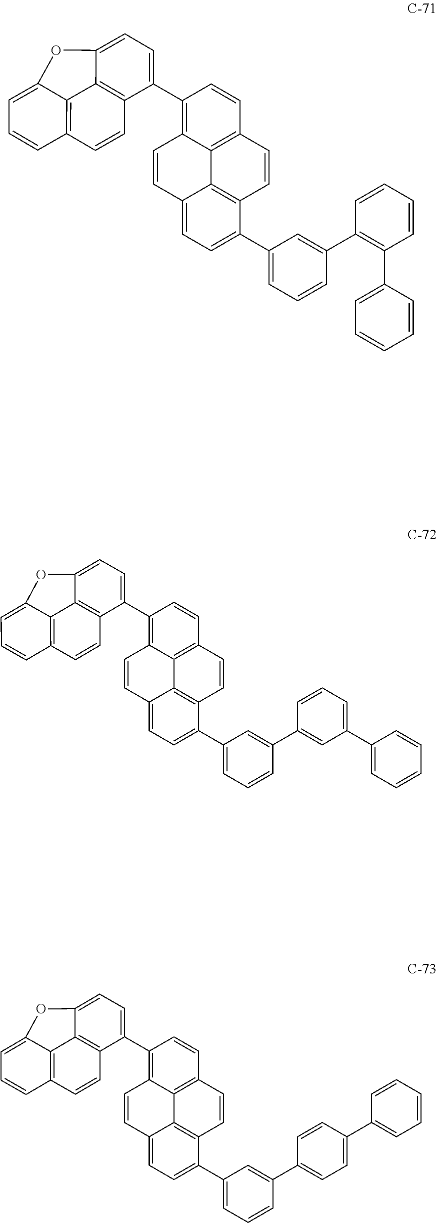 [SMILES: c1ccc(-c2ccccc2-c2cccc(-c3ccc4ccc5c(-c6ccc7oc8cccc9ccc6c7c98)ccc6ccc3c4c65)c2)cc1 ; c1ccc(-c2cccc(-c3cccc(-c4ccc5ccc6c(-c7ccc8oc9cccc%10ccc7c8c%109)ccc7ccc4c5c76)c3)c2)cc1 ; c1ccc(-c2ccc(-c3cccc(-c4ccc5ccc6c(-c7ccc8oc9cccc%10ccc7c8c%109)ccc7ccc4c5c76)c3)cc2)cc1] 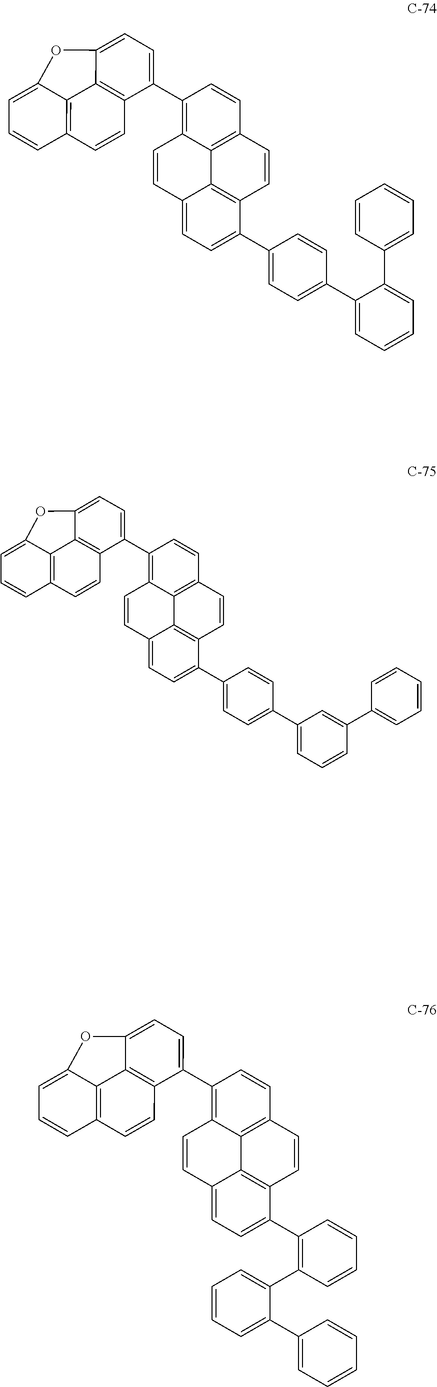 [SMILES: c1ccc(-c2ccccc2-c2ccc(-c3ccc4ccc5c(-c6ccc7oc8cccc9ccc6c7c98)ccc6ccc3c4c65)cc2)cc1 ; c1ccc(-c2cccc(-c3ccc(-c4ccc5ccc6c(-c7ccc8oc9cccc%10ccc7c8c%109)ccc7ccc4c5c76)cc3)c2)cc1 ; c1ccc(-c2ccccc2-c2ccccc2-c2ccc3ccc4c(-c5ccc6oc7cccc8ccc5c6c87)ccc5ccc2c3c54)cc1] 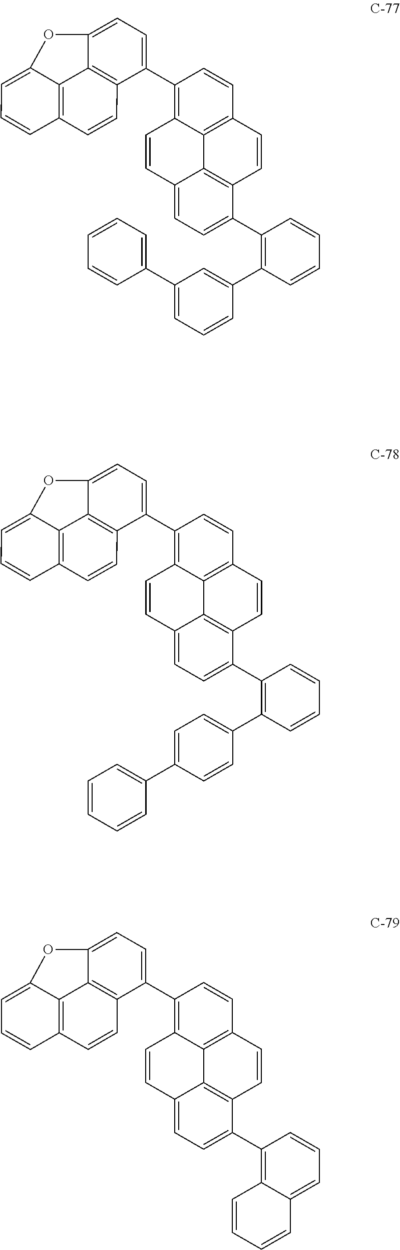 [SMILES: c1ccc(-c2cccc(-c3ccccc3-c3ccc4ccc5c(-c6ccc7oc8cccc9ccc6c7c98)ccc6ccc3c4c65)c2)cc1 ; c1ccc(-c2ccc(-c3ccccc3-c3ccc4ccc5c(-c6ccc7oc8cccc9ccc6c7c98)ccc6ccc3c4c65)cc2)cc1 ; c1ccc2c(-c3ccc4ccc5c(-c6ccc7oc8cccc9ccc6c7c98)ccc6ccc3c4c65)cccc2c1] 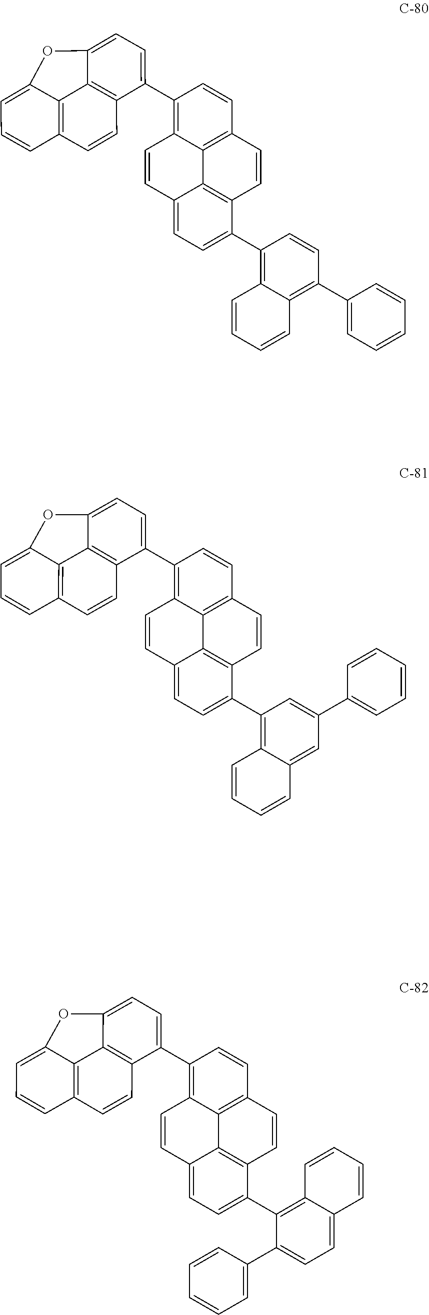 [SMILES: c1ccc(-c2ccc(-c3ccc4ccc5c(-c6ccc7oc8cccc9ccc6c7c98)ccc6ccc3c4c65)c3ccccc23)cc1 ; c1ccc(-c2cc(-c3ccc4ccc5c(-c6ccc7oc8cccc9ccc6c7c98)ccc6ccc3c4c65)c3ccccc3c2)cc1 ; c1ccc(-c2ccc3ccccc3c2-c2ccc3ccc4c(-c5ccc6oc7cccc8ccc5c6c87)ccc5ccc2c3c54)cc1] 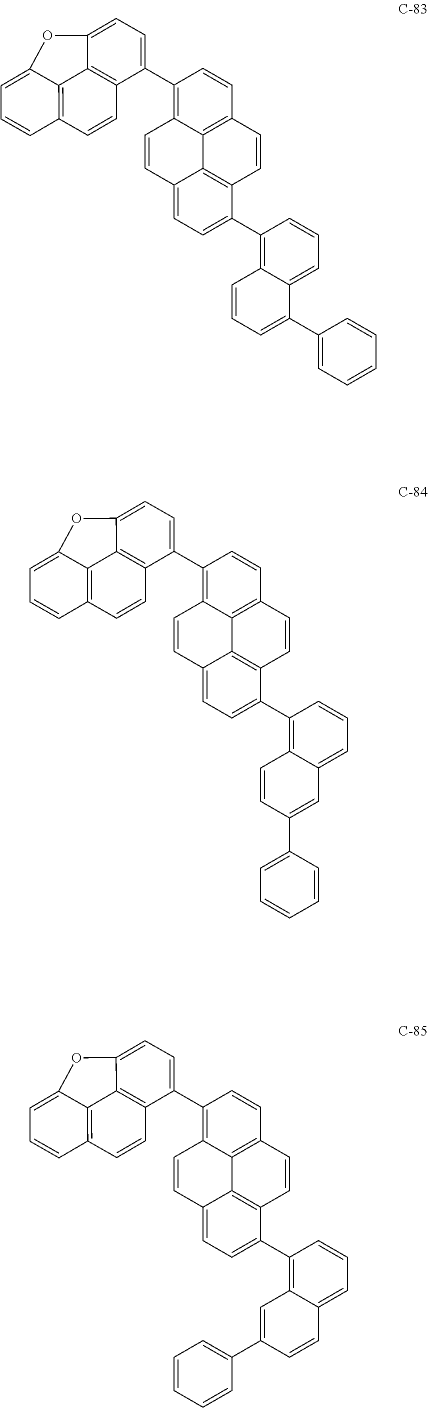 [SMILES: c1ccc(-c2cccc3c(-c4ccc5ccc6c(-c7ccc8oc9cccc%10ccc7c8c%109)ccc7ccc4c5c76)cccc23)cc1 ; c1ccc(-c2ccc3c(-c4ccc5ccc6c(-c7ccc8oc9cccc%10ccc7c8c%109)ccc7ccc4c5c76)cccc3c2)cc1 ; c1ccc(-c2ccc3cccc(-c4ccc5ccc6c(-c7ccc8oc9cccc%10ccc7c8c%109)ccc7ccc4c5c76)c3c2)cc1] 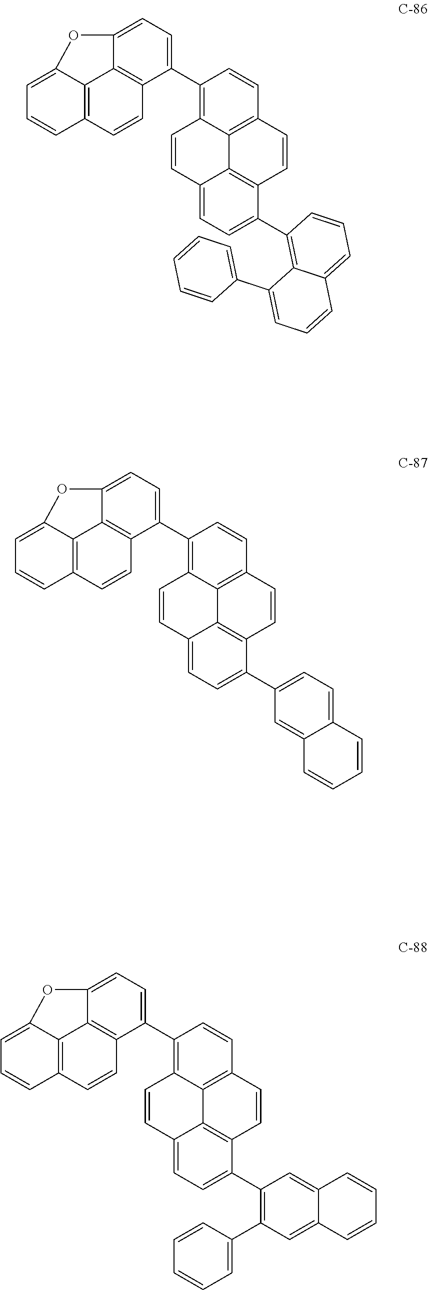 [SMILES: c1ccc(-c2cccc3cccc(-c4ccc5ccc6c(-c7ccc8oc9cccc%10ccc7c8c%109)ccc7ccc4c5c76)c23)cc1 ; c1ccc2cc(-c3ccc4ccc5c(-c6ccc7oc8cccc9ccc6c7c98)ccc6ccc3c4c65)ccc2c1 ; c1ccc(-c2cc3ccccc3cc2-c2ccc3ccc4c(-c5ccc6oc7cccc8ccc5c6c87)ccc5ccc2c3c54)cc1] 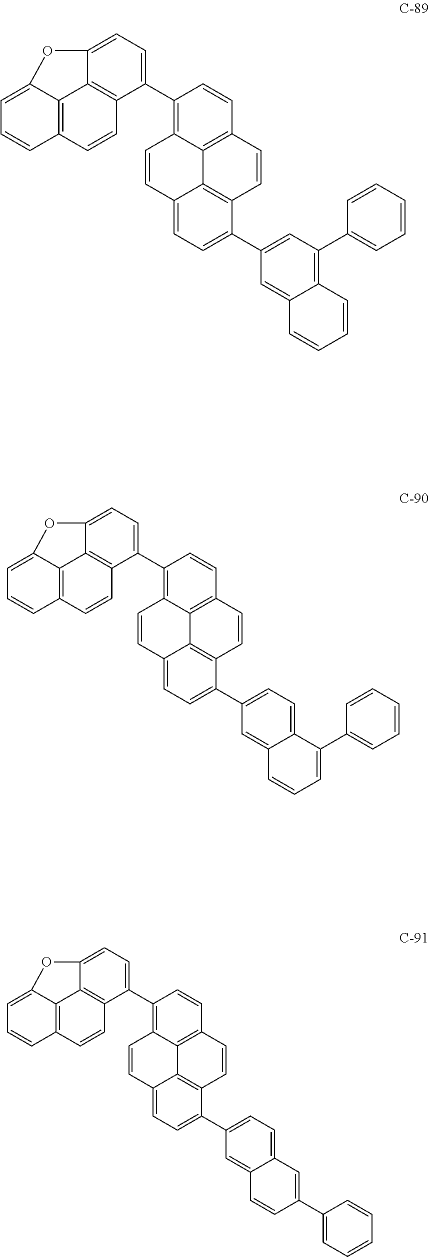 [SMILES: c1ccc(-c2cc(-c3ccc4ccc5c(-c6ccc7oc8cccc9ccc6c7c98)ccc6ccc3c4c65)cc3ccccc23)cc1 ; c1ccc(-c2cccc3cc(-c4ccc5ccc6c(-c7ccc8oc9cccc%10ccc7c8c%109)ccc7ccc4c5c76)ccc23)cc1 ; c1ccc(-c2ccc3cc(-c4ccc5ccc6c(-c7ccc8oc9cccc%10ccc7c8c%109)ccc7ccc4c5c76)ccc3c2)cc1] 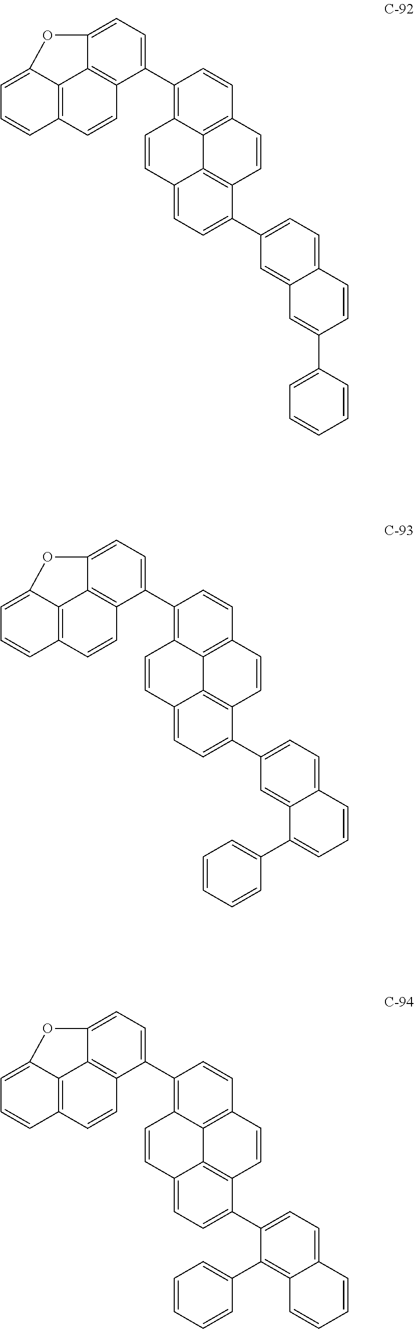 [SMILES: c1ccc(-c2ccc3ccc(-c4ccc5ccc6c(-c7ccc8oc9cccc%10ccc7c8c%109)ccc7ccc4c5c76)cc3c2)cc1 ; c1ccc(-c2cccc3ccc(-c4ccc5ccc6c(-c7ccc8oc9cccc%10ccc7c8c%109)ccc7ccc4c5c76)cc23)cc1 ; c1ccc(-c2c(-c3ccc4ccc5c(-c6ccc7oc8cccc9ccc6c7c98)ccc6ccc3c4c65)ccc3ccccc23)cc1] 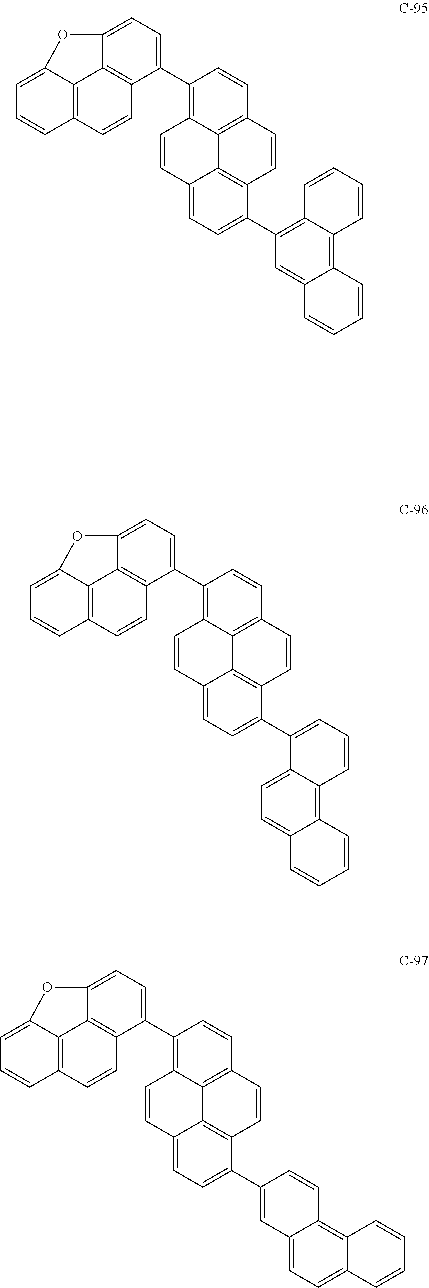 [SMILES: c1ccc2c(c1)cc(-c1ccc3ccc4c(-c5ccc6oc7cccc8ccc5c6c87)ccc5ccc1c3c54)c1ccccc12 ; c1ccc2c(c1)ccc1c(-c3ccc4ccc5c(-c6ccc7oc8cccc9ccc6c7c98)ccc6ccc3c4c65)cccc12 ; c1ccc2c(c1)ccc1cc(-c3ccc4ccc5c(-c6ccc7oc8cccc9ccc6c7c98)ccc6ccc3c4c65)ccc12] 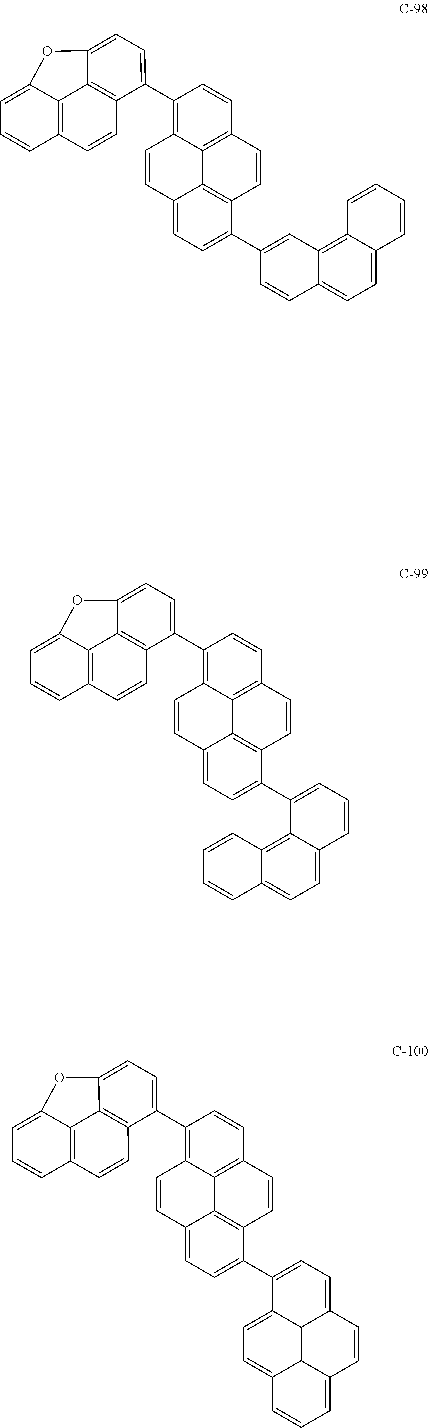 [SMILES: c1ccc2c(c1)ccc1ccc(-c3ccc4ccc5c(-c6ccc7oc8cccc9ccc6c7c98)ccc6ccc3c4c65)cc12 ; c1ccc2c(c1)ccc1cccc(-c3ccc4ccc5c(-c6ccc7oc8cccc9ccc6c7c98)ccc6ccc3c4c65)c12 ; C1=CC2=CC=C3C(c4ccc5ccc6c(-c7ccc8oc9cccc%10ccc7c8c%109)ccc7ccc4c5c76)=CC=C4C=CC(=C1)C2C43] 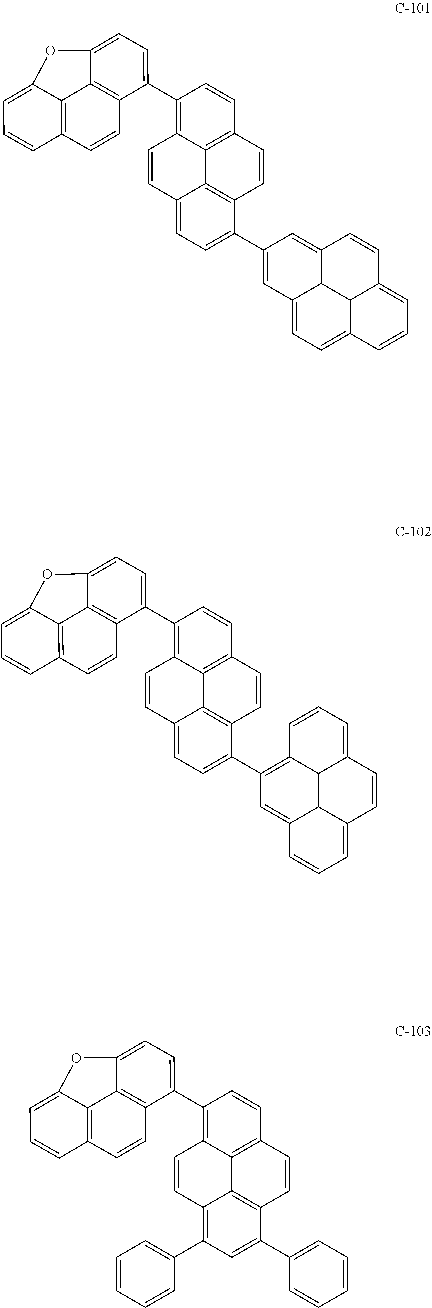 [SMILES: C1=CC2=CC=C3C=C(c4ccc5ccc6c(-c7ccc8oc9cccc%10ccc7c8c%109)ccc7ccc4c5c76)C=C4C=CC(=C1)C2C34 ; C1=CC2=CC(c3ccc4ccc5c(-c6ccc7oc8cccc9ccc6c7c98)ccc6ccc3c4c65)=C3C=CC=C4C=CC(=C1)C2C43 ; c1ccc(-c2cc(-c3ccccc3)c3ccc4c(-c5ccc6oc7cccc8ccc5c6c87)ccc5ccc2c3c54)cc1] 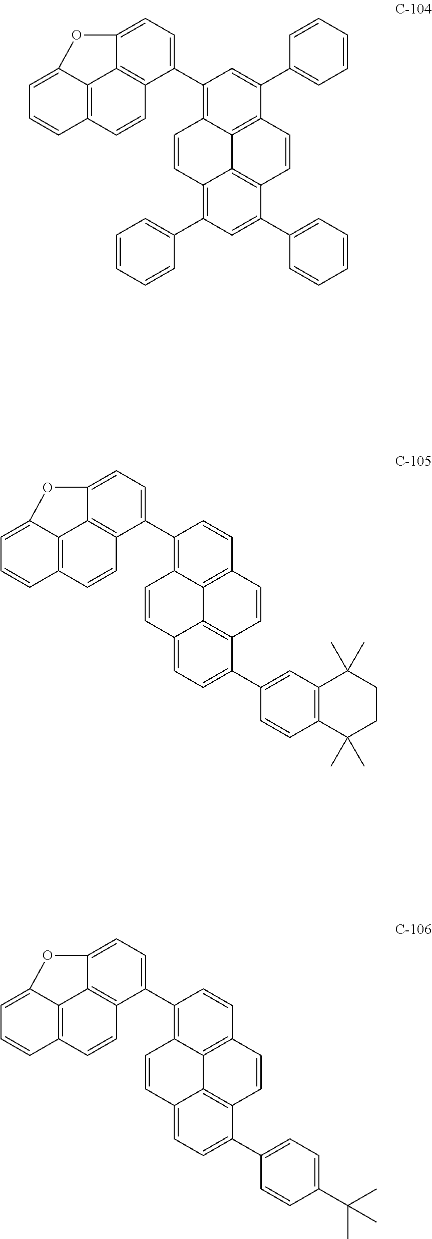 [SMILES: c1ccc(-c2cc(-c3ccccc3)c3ccc4c(-c5ccc6oc7cccc8ccc5c6c87)cc(-c5ccccc5)c5ccc2c3c54)cc1 ; CC1(C)CCC(C)(C)c2cc(-c3ccc4ccc5c(-c6ccc7oc8cccc9ccc6c7c98)ccc6ccc3c4c65)ccc21 ; CC(C)(C)c1ccc(-c2ccc3ccc4c(-c5ccc6oc7cccc8ccc5c6c87)ccc5ccc2c3c54)cc1] 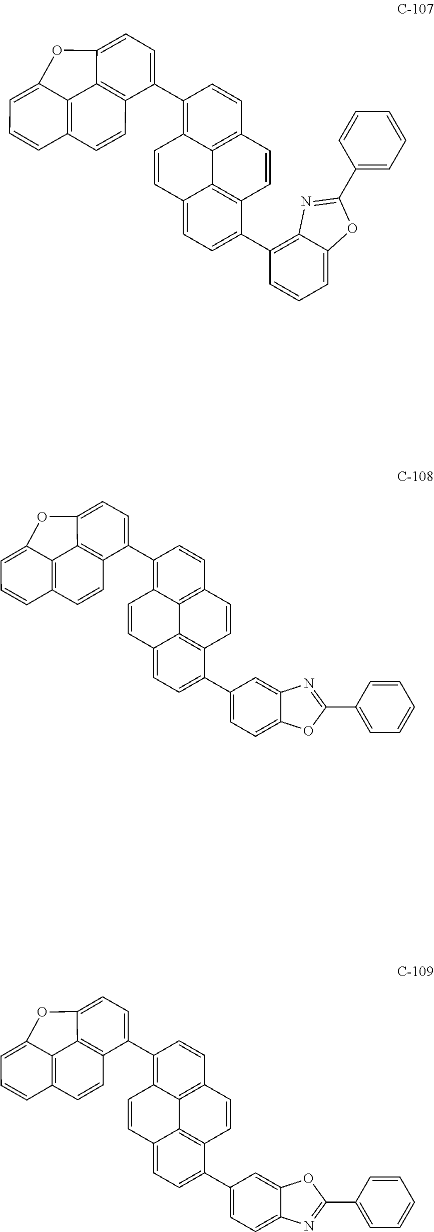 [SMILES: c1ccc(-c2nc3c(-c4ccc5ccc6c(-c7ccc8oc9cccc%10ccc7c8c%109)ccc7ccc4c5c76)cccc3o2)cc1 ; c1ccc(-c2nc3cc(-c4ccc5ccc6c(-c7ccc8oc9cccc%10ccc7c8c%109)ccc7ccc4c5c76)ccc3o2)cc1 ; c1ccc(-c2nc3ccc(-c4ccc5ccc6c(-c7ccc8oc9cccc%10ccc7c8c%109)ccc7ccc4c5c76)cc3o2)cc1] 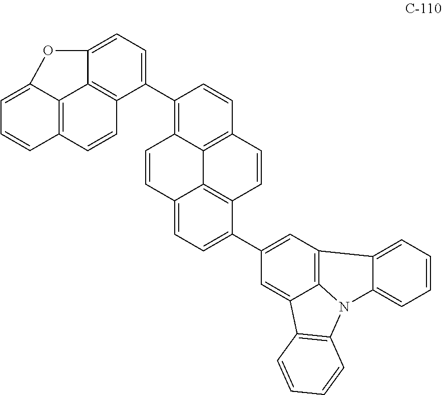 [SMILES: c1cc2ccc3c(-c4ccc5ccc6c(-c7cc8c9ccccc9n9c%10ccccc%10c(c7)c89)ccc7ccc4c5c76)ccc4oc(c1)c2c43] 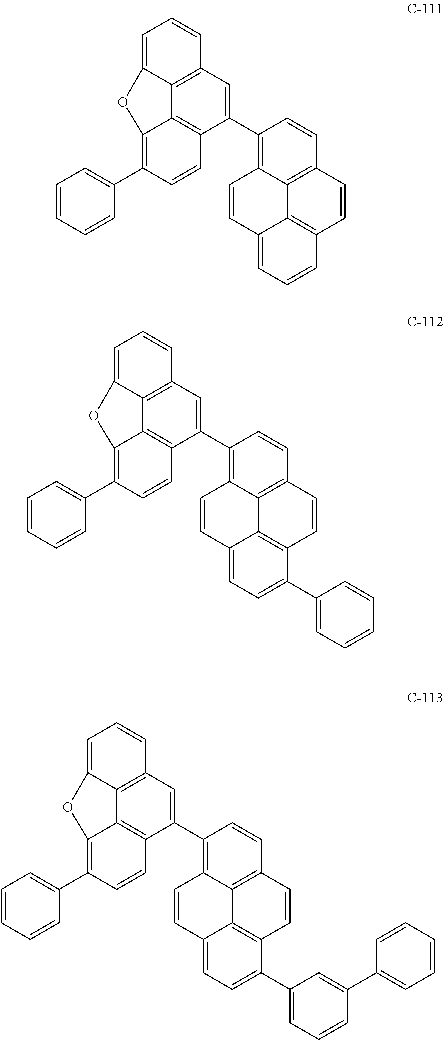 [SMILES: c1ccc(-c2ccc3c(-c4ccc5ccc6cccc7ccc4c5c67)cc4cccc5oc2c3c45)cc1 ; c1ccc(-c2ccc3ccc4c(-c5cc6cccc7oc8c(-c9ccccc9)ccc5c8c67)ccc5ccc2c3c54)cc1 ; c1ccc(-c2cccc(-c3ccc4ccc5c(-c6cc7cccc8oc9c(-c%10ccccc%10)ccc6c9c78)ccc6ccc3c4c65)c2)cc1] 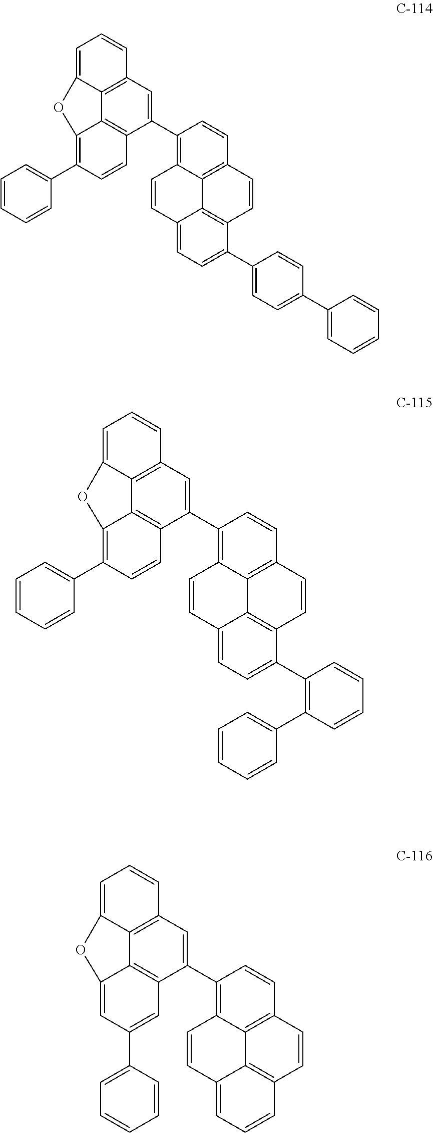 [SMILES: c1ccc(-c2ccc(-c3ccc4ccc5c(-c6cc7cccc8oc9c(-c%10ccccc%10)ccc6c9c78)ccc6ccc3c4c65)cc2)cc1 ; c1ccc(-c2ccccc2-c2ccc3ccc4c(-c5cc6cccc7oc8c(-c9ccccc9)ccc5c8c67)ccc5ccc2c3c54)cc1 ; c1ccc(-c2cc3oc4cccc5cc(-c6ccc7ccc8cccc9ccc6c7c89)c(c2)c3c54)cc1] 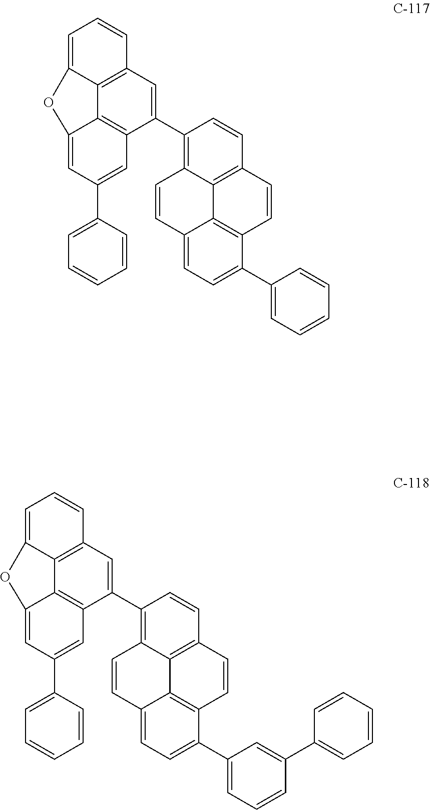 [SMILES: c1ccc(-c2cc3oc4cccc5cc(-c6ccc7ccc8c(-c9ccccc9)ccc9ccc6c7c98)c(c2)c3c54)cc1 ; c1ccc(-c2cccc(-c3ccc4ccc5c(-c6cc7cccc8oc9cc(-c%10ccccc%10)cc6c9c78)ccc6ccc3c4c65)c2)cc1] 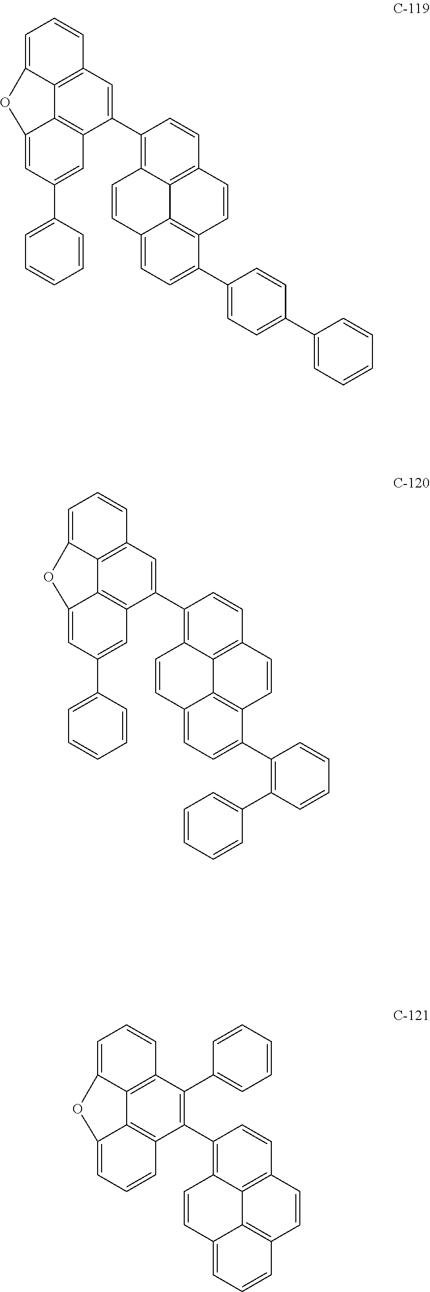 [SMILES: c1ccc(-c2ccc(-c3ccc4ccc5c(-c6cc7cccc8oc9cc(-c%10ccccc%10)cc6c9c78)ccc6ccc3c4c65)cc2)cc1 ; c1ccc(-c2cc3oc4cccc5cc(-c6ccc7ccc8c(-c9ccccc9-c9ccccc9)ccc9ccc6c7c98)c(c2)c3c54)cc1 ; c1ccc(-c2c(-c3ccc4ccc5cccc6ccc3c4c56)c3cccc4oc5cccc2c5c43)cc1] 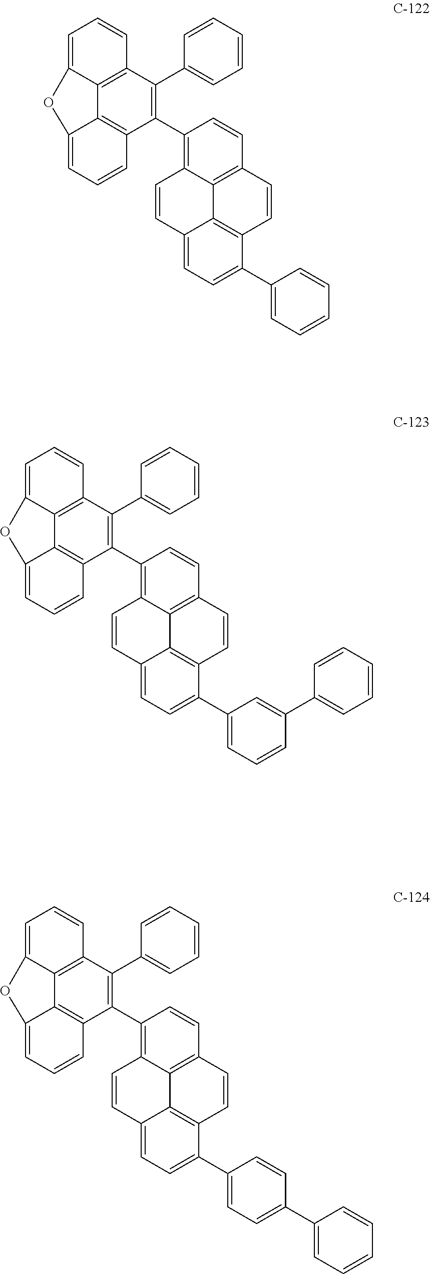 [SMILES: c1ccc(-c2ccc3ccc4c(-c5c(-c6ccccc6)c6cccc7oc8cccc5c8c76)ccc5ccc2c3c54)cc1 ; c1ccc(-c2cccc(-c3ccc4ccc5c(-c6c(-c7ccccc7)c7cccc8oc9cccc6c9c87)ccc6ccc3c4c65)c2)cc1 ; c1ccc(-c2ccc(-c3ccc4ccc5c(-c6c(-c7ccccc7)c7cccc8oc9cccc6c9c87)ccc6ccc3c4c65)cc2)cc1] 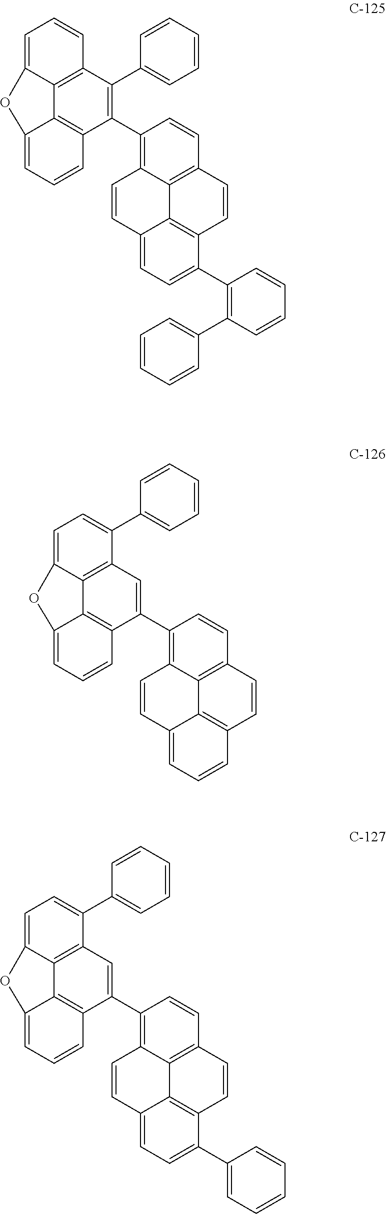 [SMILES: c1ccc(-c2ccccc2-c2ccc3ccc4c(-c5c(-c6ccccc6)c6cccc7oc8cccc5c8c76)ccc5ccc2c3c54)cc1 ; c1ccc(-c2ccc3oc4cccc5c(-c6ccc7ccc8cccc9ccc6c7c89)cc2c3c45)cc1 ; c1ccc(-c2ccc3ccc4c(-c5cc6c(-c7ccccc7)ccc7oc8cccc5c8c76)ccc5ccc2c3c54)cc1] 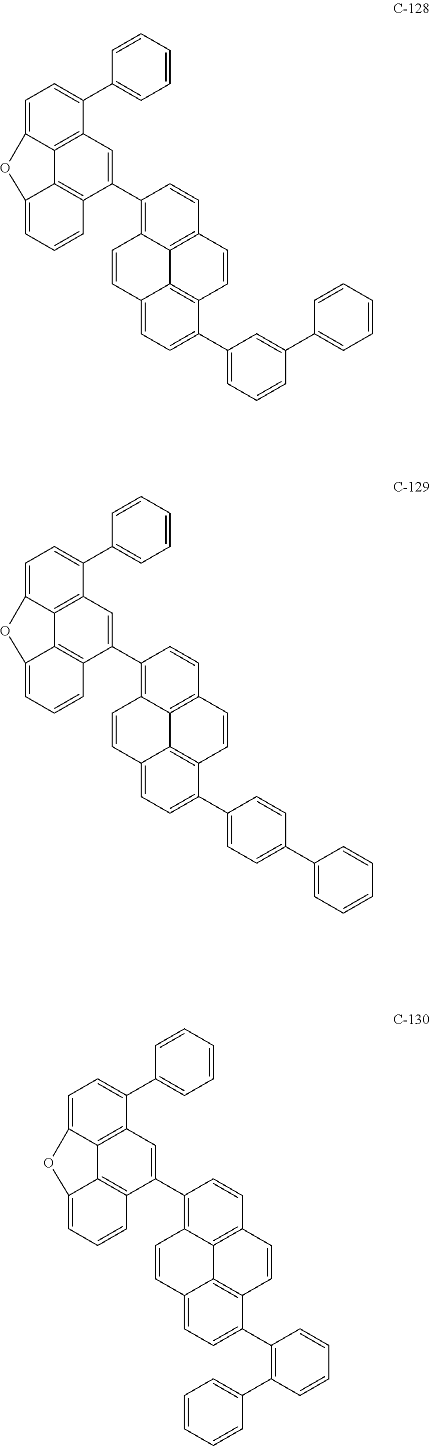 [SMILES: c1ccc(-c2cccc(-c3ccc4ccc5c(-c6cc7c(-c8ccccc8)ccc8oc9cccc6c9c87)ccc6ccc3c4c65)c2)cc1 ; c1ccc(-c2ccc(-c3ccc4ccc5c(-c6cc7c(-c8ccccc8)ccc8oc9cccc6c9c87)ccc6ccc3c4c65)cc2)cc1 ; c1ccc(-c2ccccc2-c2ccc3ccc4c(-c5cc6c(-c7ccccc7)ccc7oc8cccc5c8c76)ccc5ccc2c3c54)cc1] 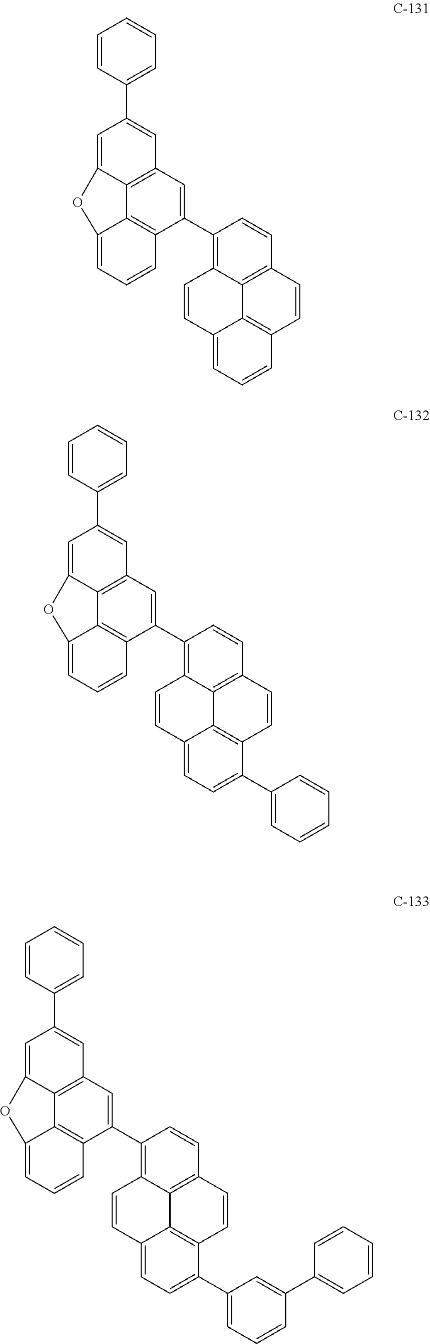 [SMILES: c1ccc(-c2cc3cc(-c4ccc5ccc6cccc7ccc4c5c67)c4cccc5oc(c2)c3c54)cc1 ; c1ccc(-c2cc3cc(-c4ccc5ccc6c(-c7ccccc7)ccc7ccc4c5c76)c4cccc5oc(c2)c3c54)cc1 ; c1ccc(-c2cccc(-c3ccc4ccc5c(-c6cc7cc(-c8ccccc8)cc8oc9cccc6c9c78)ccc6ccc3c4c65)c2)cc1] 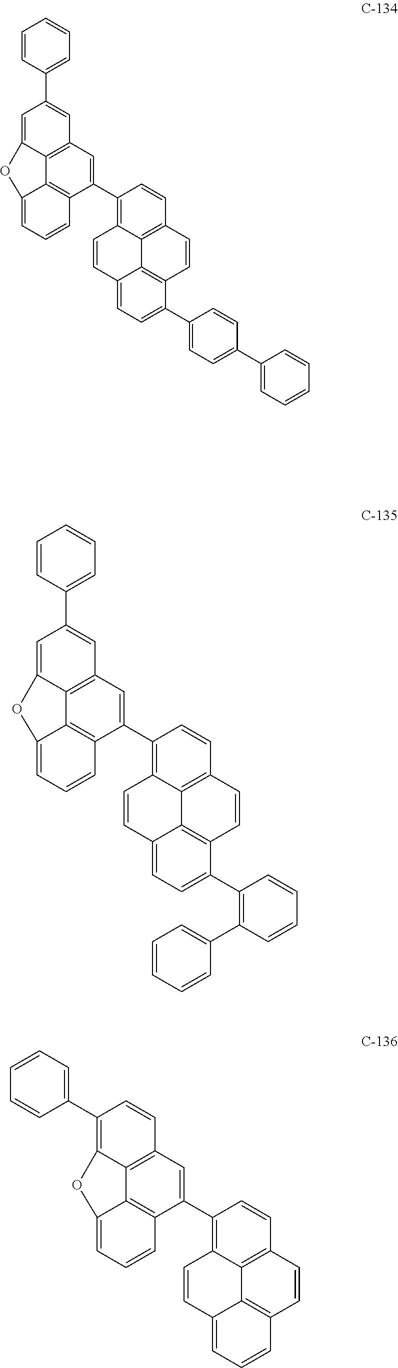 [SMILES: c1ccc(-c2ccc(-c3ccc4ccc5c(-c6cc7cc(-c8ccccc8)cc8oc9cccc6c9c78)ccc6ccc3c4c65)cc2)cc1 ; c1ccc(-c2cc3cc(-c4ccc5ccc6c(-c7ccccc7-c7ccccc7)ccc7ccc4c5c76)c4cccc5oc(c2)c3c54)cc1 ; c1ccc(-c2ccc3cc(-c4ccc5ccc6cccc7ccc4c5c67)c4cccc5oc2c3c54)cc1] 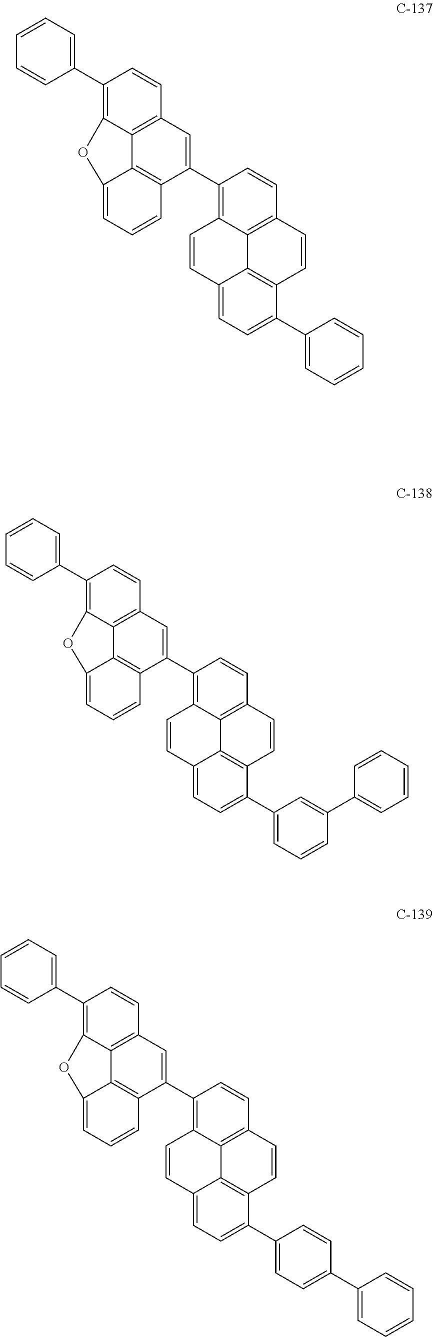 [SMILES: c1ccc(-c2ccc3ccc4c(-c5cc6ccc(-c7ccccc7)c7oc8cccc5c8c67)ccc5ccc2c3c54)cc1 ; c1ccc(-c2cccc(-c3ccc4ccc5c(-c6cc7ccc(-c8ccccc8)c8oc9cccc6c9c78)ccc6ccc3c4c65)c2)cc1 ; c1ccc(-c2ccc(-c3ccc4ccc5c(-c6cc7ccc(-c8ccccc8)c8oc9cccc6c9c78)ccc6ccc3c4c65)cc2)cc1] 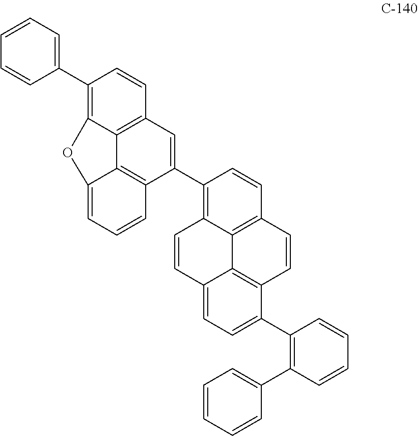 [SMILES: c1ccc(-c2ccccc2-c2ccc3ccc4c(-c5cc6ccc(-c7ccccc7)c7oc8cccc5c8c67)ccc5ccc2c3c54)cc1] 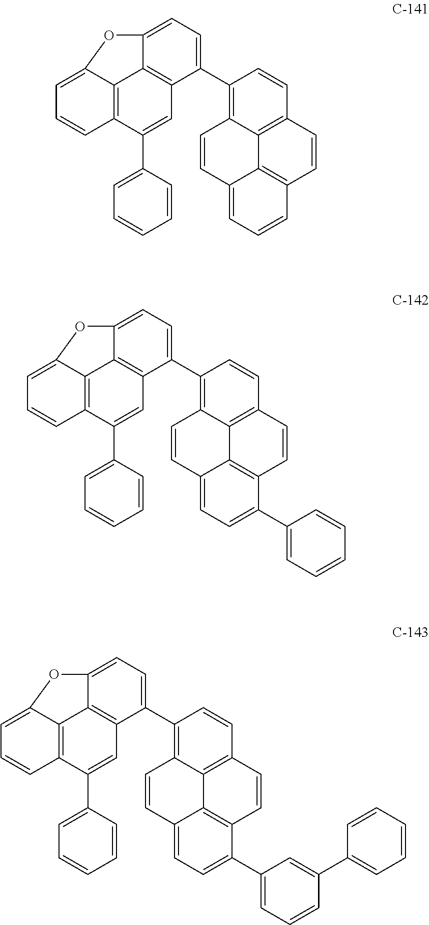 [SMILES: c1ccc(-c2cc3c(-c4ccc5ccc6cccc7ccc4c5c67)ccc4oc5cccc2c5c43)cc1 ; c1ccc(-c2ccc3ccc4c(-c5ccc6oc7cccc8c(-c9ccccc9)cc5c6c78)ccc5ccc2c3c54)cc1 ; c1ccc(-c2cccc(-c3ccc4ccc5c(-c6ccc7oc8cccc9c(-c%10ccccc%10)cc6c7c89)ccc6ccc3c4c65)c2)cc1] 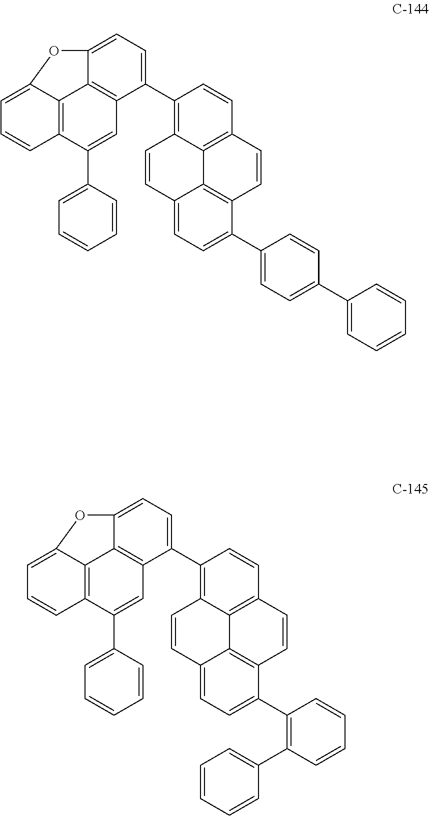 [SMILES: c1ccc(-c2ccc(-c3ccc4ccc5c(-c6ccc7oc8cccc9c(-c%10ccccc%10)cc6c7c89)ccc6ccc3c4c65)cc2)cc1 ; c1ccc(-c2ccccc2-c2ccc3ccc4c(-c5ccc6oc7cccc8c(-c9ccccc9)cc5c6c78)ccc5ccc2c3c54)cc1] 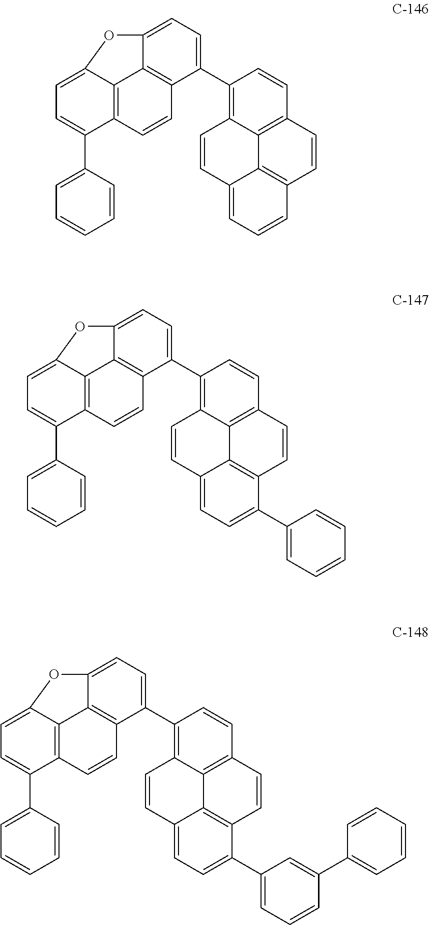 [SMILES: c1ccc(-c2ccc3oc4ccc(-c5ccc6ccc7cccc8ccc5c6c78)c5ccc2c3c45)cc1 ; c1ccc(-c2ccc3ccc4c(-c5ccc6oc7ccc(-c8ccccc8)c8ccc5c6c78)ccc5ccc2c3c54)cc1 ; c1ccc(-c2cccc(-c3ccc4ccc5c(-c6ccc7oc8ccc(-c9ccccc9)c9ccc6c7c89)ccc6ccc3c4c65)c2)cc1] 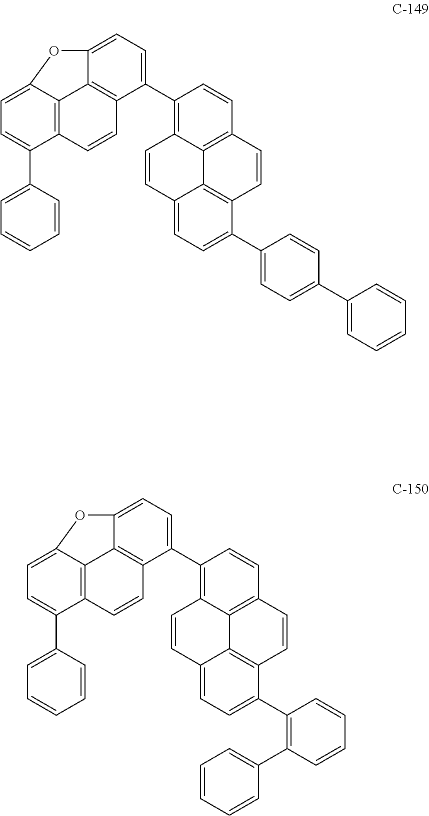 [SMILES: c1ccc(-c2ccc(-c3ccc4ccc5c(-c6ccc7oc8ccc(-c9ccccc9)c9ccc6c7c89)ccc6ccc3c4c65)cc2)cc1 ; c1ccc(-c2ccccc2-c2ccc3ccc4c(-c5ccc6oc7ccc(-c8ccccc8)c8ccc5c6c78)ccc5ccc2c3c54)cc1] 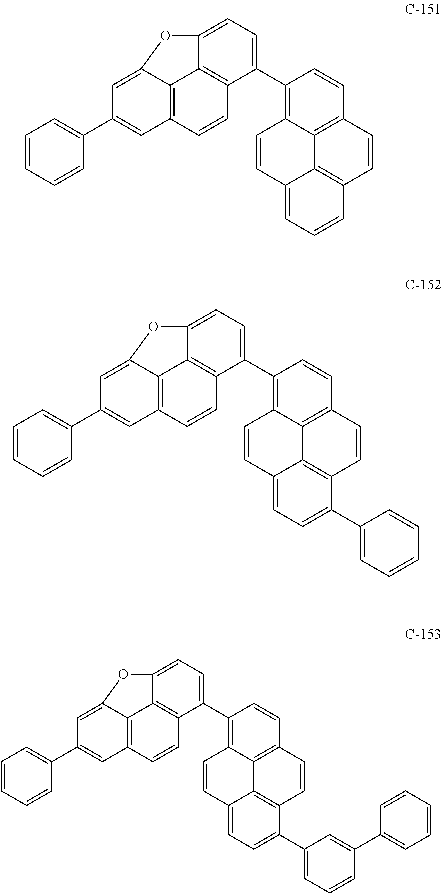 [SMILES: c1ccc(-c2cc3ccc4c(-c5ccc6ccc7cccc8ccc5c6c78)ccc5oc(c2)c3c54)cc1 ; c1ccc(-c2cc3ccc4c(-c5ccc6ccc7c(-c8ccccc8)ccc8ccc5c6c87)ccc5oc(c2)c3c54)cc1 ; c1ccc(-c2cccc(-c3ccc4ccc5c(-c6ccc7oc8cc(-c9ccccc9)cc9ccc6c7c98)ccc6ccc3c4c65)c2)cc1] 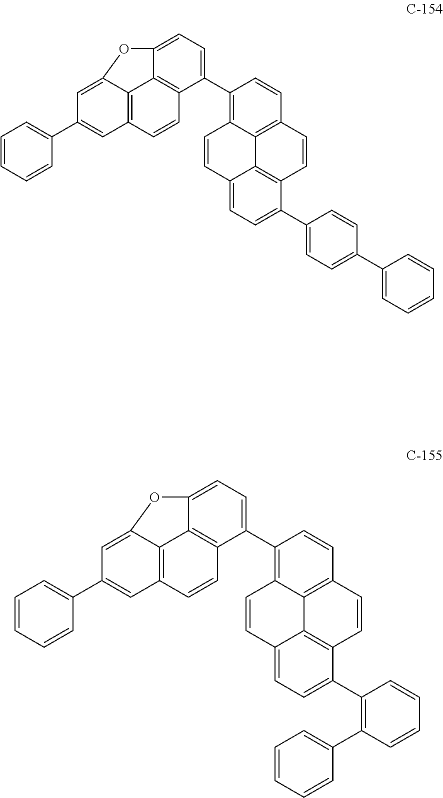 [SMILES: c1ccc(-c2ccc(-c3ccc4ccc5c(-c6ccc7oc8cc(-c9ccccc9)cc9ccc6c7c98)ccc6ccc3c4c65)cc2)cc1 ; c1ccc(-c2cc3ccc4c(-c5ccc6ccc7c(-c8ccccc8-c8ccccc8)ccc8ccc5c6c87)ccc5oc(c2)c3c54)cc1] 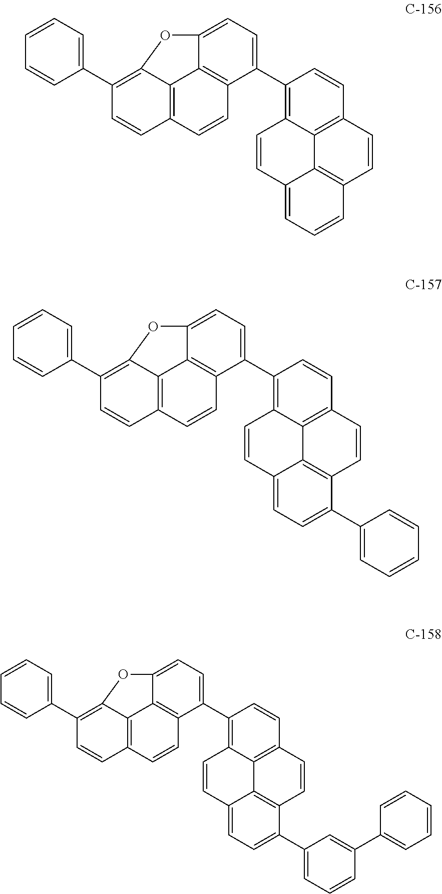 [SMILES: c1ccc(-c2ccc3ccc4c(-c5ccc6ccc7cccc8ccc5c6c78)ccc5oc2c3c54)cc1 ; c1ccc(-c2ccc3ccc4c(-c5ccc6oc7c(-c8ccccc8)ccc8ccc5c6c87)ccc5ccc2c3c54)cc1 ; c1ccc(-c2cccc(-c3ccc4ccc5c(-c6ccc7oc8c(-c9ccccc9)ccc9ccc6c7c98)ccc6ccc3c4c65)c2)cc1] 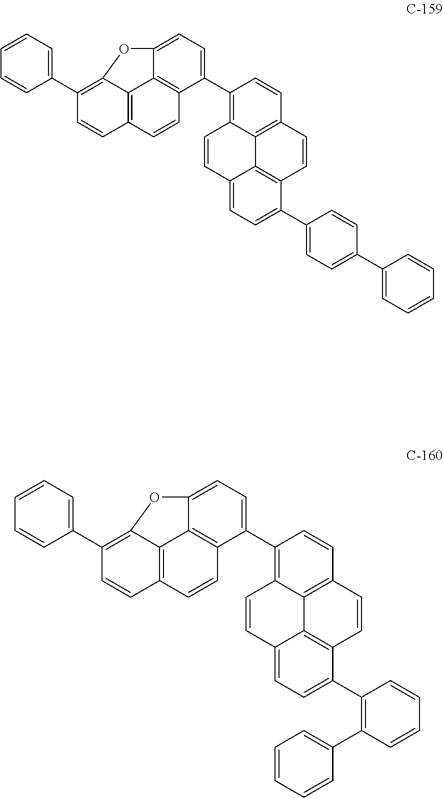 [SMILES: c1ccc(-c2ccc(-c3ccc4ccc5c(-c6ccc7oc8c(-c9ccccc9)ccc9ccc6c7c98)ccc6ccc3c4c65)cc2)cc1 ; c1ccc(-c2ccccc2-c2ccc3ccc4c(-c5ccc6oc7c(-c8ccccc8)ccc8ccc5c6c87)ccc5ccc2c3c54)cc1] 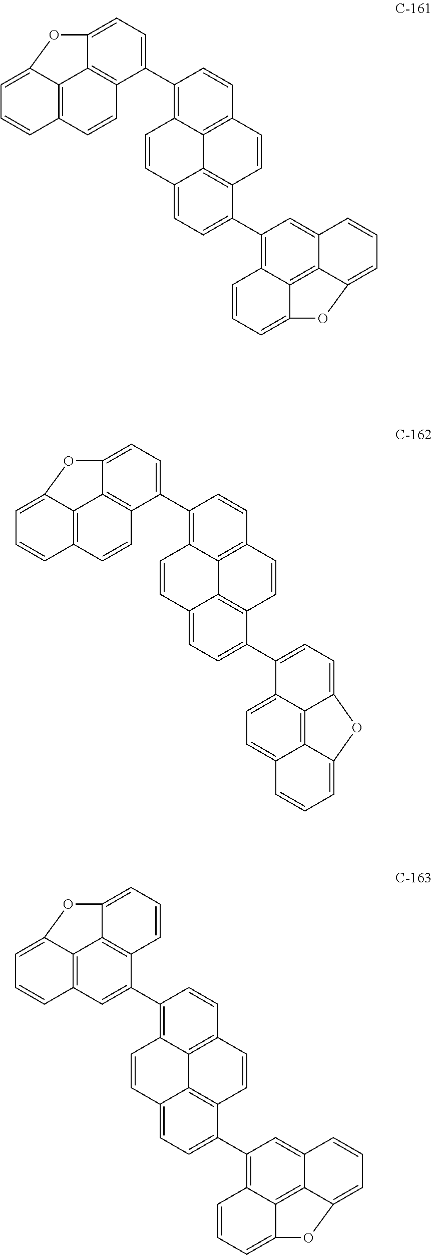 [SMILES: c1cc2ccc3c(-c4ccc5ccc6c(-c7cc8cccc9oc%10cccc7c%10c89)ccc7ccc4c5c76)ccc4oc(c1)c2c43 ; c1cc2ccc3c(-c4ccc5ccc6c(-c7ccc8oc9cccc%10ccc7c8c%109)ccc7ccc4c5c76)ccc4oc(c1)c2c43 ; c1cc2cc(-c3ccc4ccc5c(-c6cc7cccc8oc9cccc6c9c78)ccc6ccc3c4c65)c3cccc4oc(c1)c2c43] 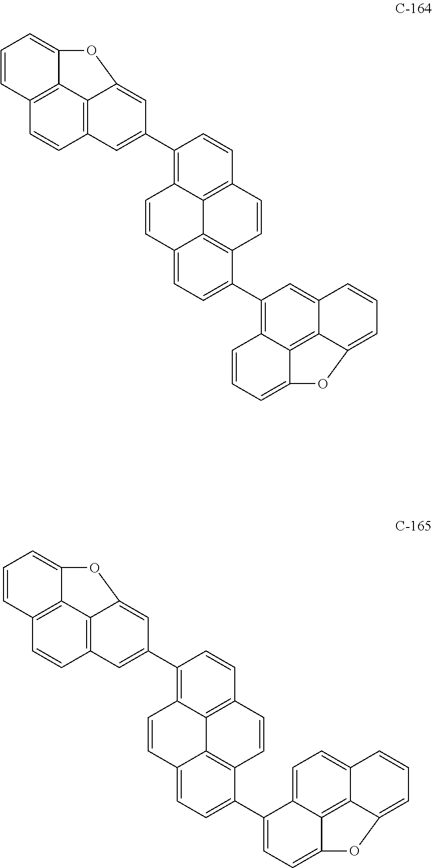 [SMILES: c1cc2ccc3cc(-c4ccc5ccc6c(-c7cc8cccc9oc%10cccc7c%10c89)ccc7ccc4c5c76)cc4oc(c1)c2c34 ; c1cc2ccc3cc(-c4ccc5ccc6c(-c7ccc8oc9cccc%10ccc7c8c%109)ccc7ccc4c5c76)cc4oc(c1)c2c34] 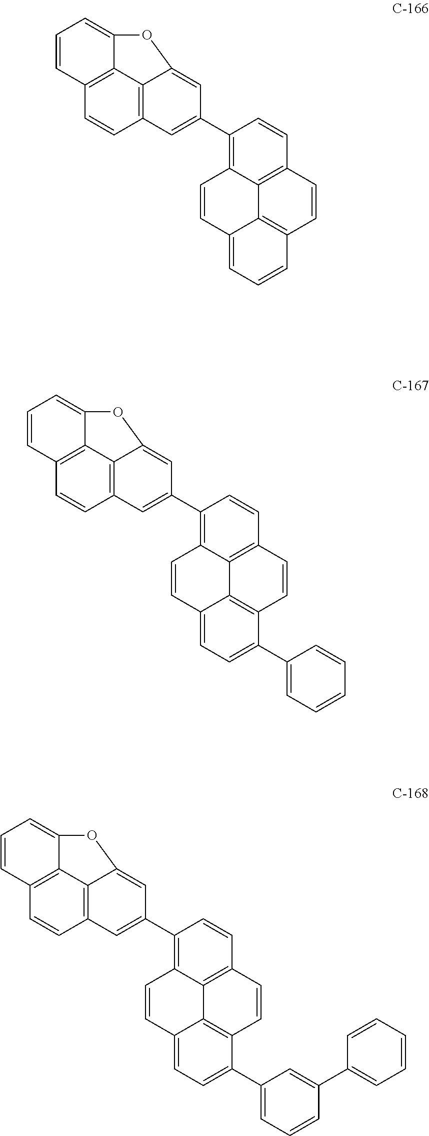 [SMILES: c1cc2ccc3ccc(-c4cc5ccc6cccc7oc(c4)c5c67)c4ccc(c1)c2c34 ; c1ccc(-c2ccc3ccc4c(-c5cc6ccc7cccc8oc(c5)c6c78)ccc5ccc2c3c54)cc1 ; c1ccc(-c2cccc(-c3ccc4ccc5c(-c6cc7ccc8cccc9oc(c6)c7c89)ccc6ccc3c4c65)c2)cc1] 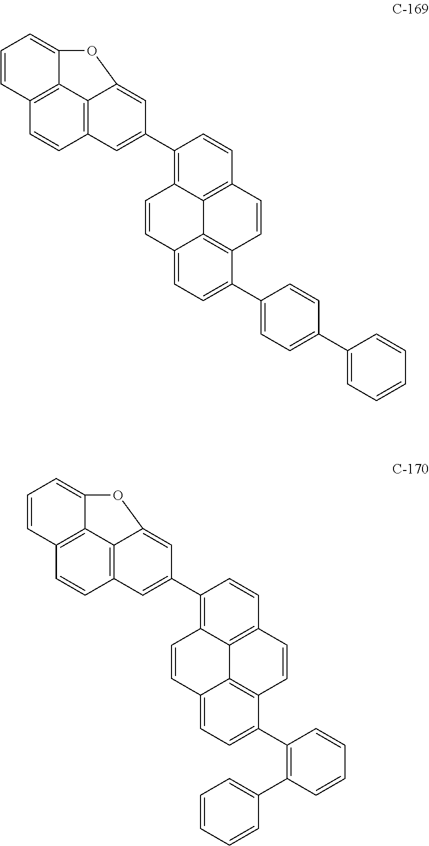 [SMILES: c1ccc(-c2ccc(-c3ccc4ccc5c(-c6cc7ccc8cccc9oc(c6)c7c89)ccc6ccc3c4c65)cc2)cc1 ; c1ccc(-c2ccccc2-c2ccc3ccc4c(-c5cc6ccc7cccc8oc(c5)c6c78)ccc5ccc2c3c54)cc1] 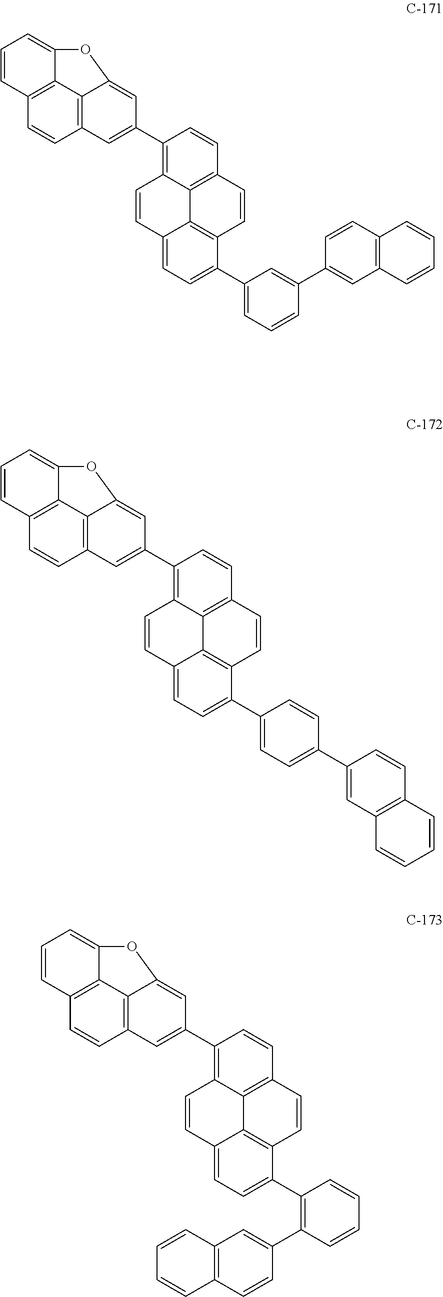 [SMILES: c1cc(-c2ccc3ccccc3c2)cc(-c2ccc3ccc4c(-c5cc6ccc7cccc8oc(c5)c6c78)ccc5ccc2c3c54)c1 ; c1ccc2cc(-c3ccc(-c4ccc5ccc6c(-c7cc8ccc9cccc%10oc(c7)c8c9%10)ccc7ccc4c5c76)cc3)ccc2c1 ; c1ccc(-c2ccc3ccc4c(-c5cc6ccc7cccc8oc(c5)c6c78)ccc5ccc2c3c54)c(-c2ccc3ccccc3c2)c1] 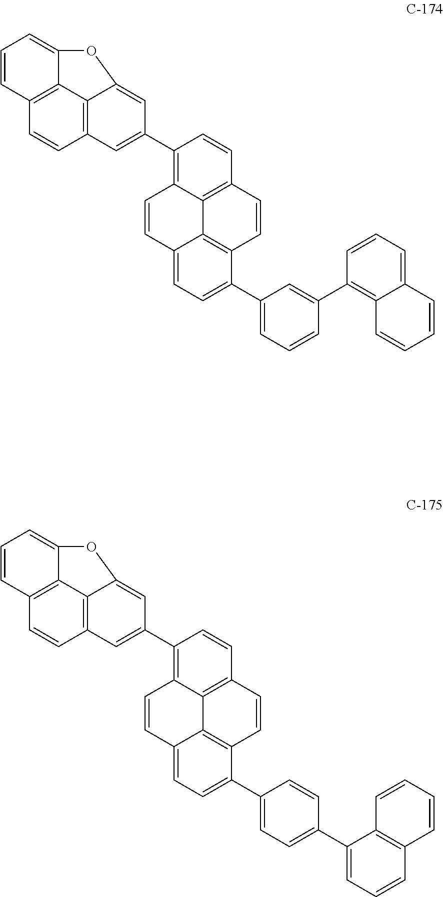 [SMILES: c1cc(-c2cccc3ccccc23)cc(-c2ccc3ccc4c(-c5cc6ccc7cccc8oc(c5)c6c78)ccc5ccc2c3c54)c1 ; c1ccc2c(-c3ccc(-c4ccc5ccc6c(-c7cc8ccc9cccc%10oc(c7)c8c9%10)ccc7ccc4c5c76)cc3)cccc2c1] 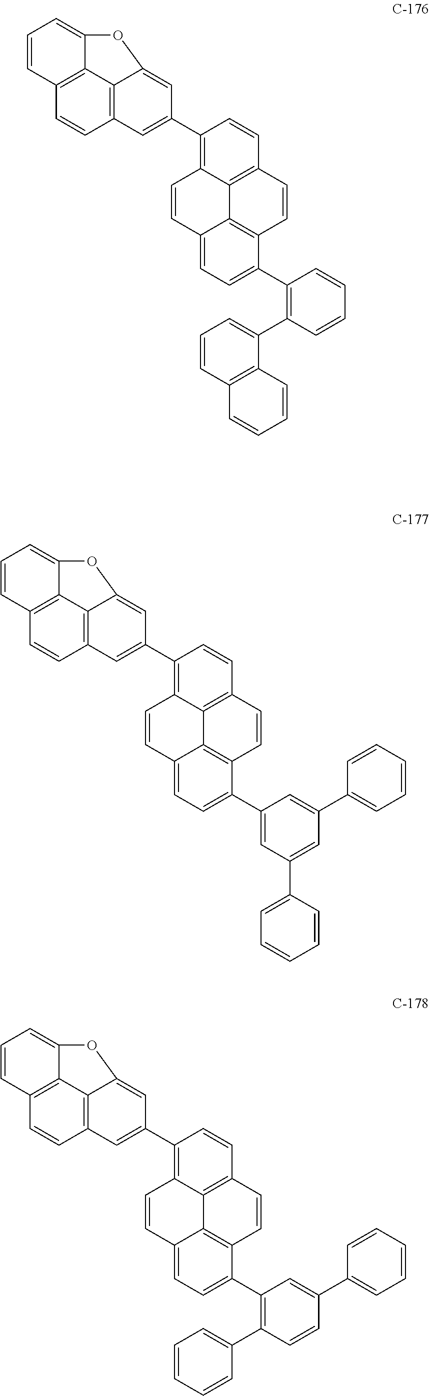 [SMILES: c1ccc(-c2ccc3ccc4c(-c5cc6ccc7cccc8oc(c5)c6c78)ccc5ccc2c3c54)c(-c2cccc3ccccc23)c1 ; c1ccc(-c2cc(-c3ccccc3)cc(-c3ccc4ccc5c(-c6cc7ccc8cccc9oc(c6)c7c89)ccc6ccc3c4c65)c2)cc1 ; c1ccc(-c2ccc(-c3ccccc3)c(-c3ccc4ccc5c(-c6cc7ccc8cccc9oc(c6)c7c89)ccc6ccc3c4c65)c2)cc1] 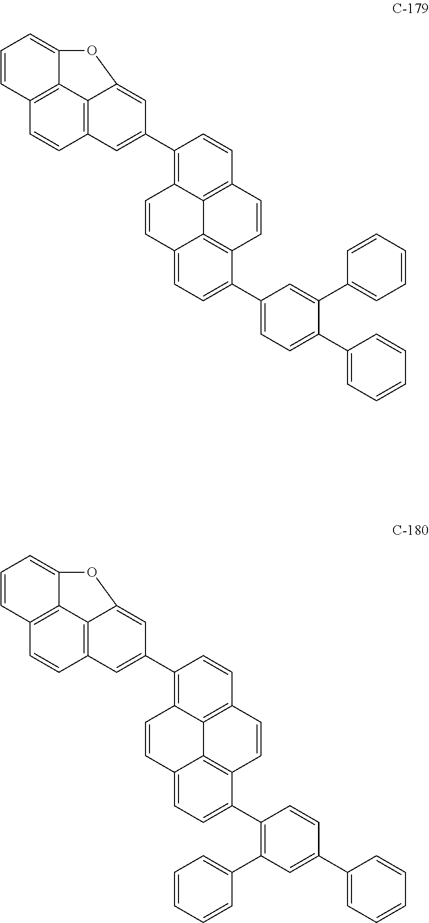 [SMILES: c1ccc(-c2ccc(-c3ccc4ccc5c(-c6cc7ccc8cccc9oc(c6)c7c89)ccc6ccc3c4c65)cc2-c2ccccc2)cc1 ; c1ccc(-c2ccc(-c3ccc4ccc5c(-c6cc7ccc8cccc9oc(c6)c7c89)ccc6ccc3c4c65)c(-c3ccccc3)c2)cc1] 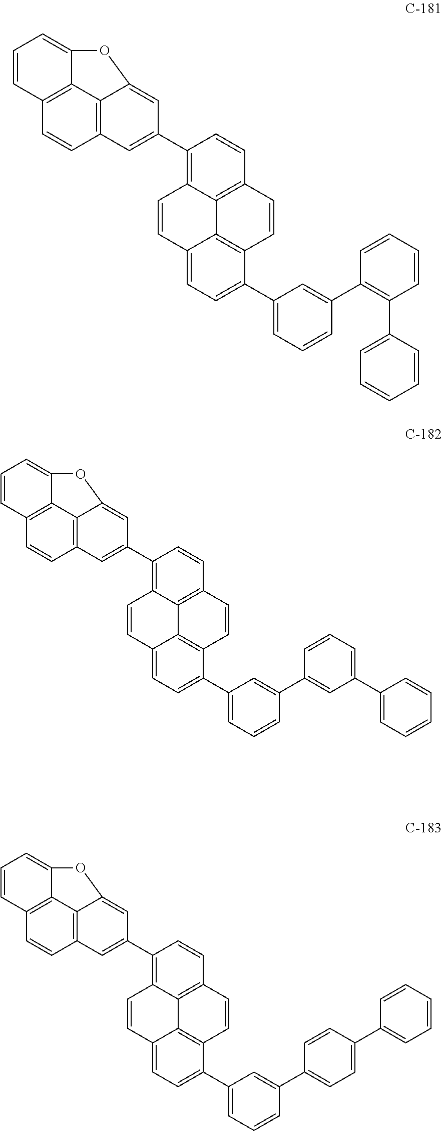 [SMILES: c1ccc(-c2ccccc2-c2cccc(-c3ccc4ccc5c(-c6cc7ccc8cccc9oc(c6)c7c89)ccc6ccc3c4c65)c2)cc1 ; c1ccc(-c2cccc(-c3cccc(-c4ccc5ccc6c(-c7cc8ccc9cccc%10oc(c7)c8c9%10)ccc7ccc4c5c76)c3)c2)cc1 ; c1ccc(-c2ccc(-c3cccc(-c4ccc5ccc6c(-c7cc8ccc9cccc%10oc(c7)c8c9%10)ccc7ccc4c5c76)c3)cc2)cc1] 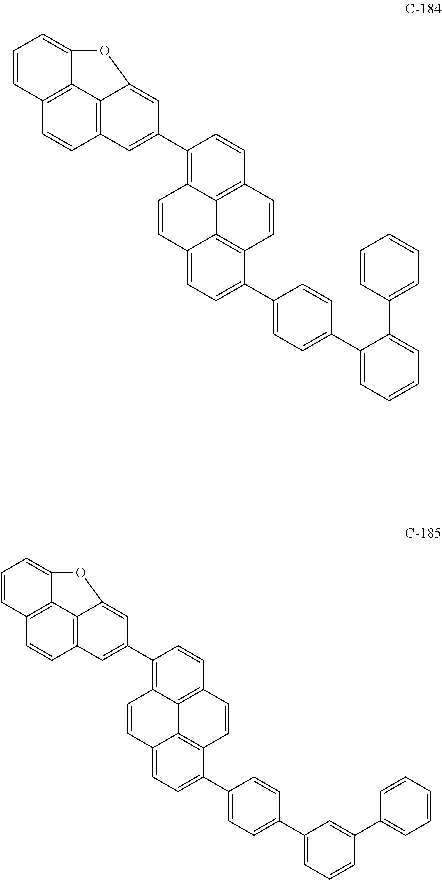 [SMILES: c1ccc(-c2ccccc2-c2ccc(-c3ccc4ccc5c(-c6cc7ccc8cccc9oc(c6)c7c89)ccc6ccc3c4c65)cc2)cc1 ; c1ccc(-c2cccc(-c3ccc(-c4ccc5ccc6c(-c7cc8ccc9cccc%10oc(c7)c8c9%10)ccc7ccc4c5c76)cc3)c2)cc1] 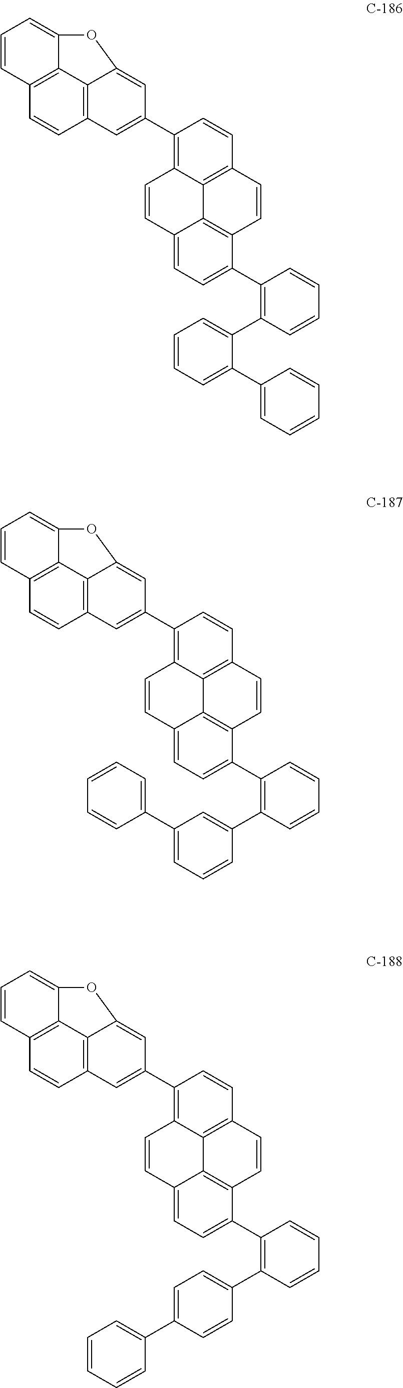 [SMILES: c1ccc(-c2ccccc2-c2ccccc2-c2ccc3ccc4c(-c5cc6ccc7cccc8oc(c5)c6c78)ccc5ccc2c3c54)cc1 ; c1ccc(-c2cccc(-c3ccccc3-c3ccc4ccc5c(-c6cc7ccc8cccc9oc(c6)c7c89)ccc6ccc3c4c65)c2)cc1 ; c1ccc(-c2ccc(-c3ccccc3-c3ccc4ccc5c(-c6cc7ccc8cccc9oc(c6)c7c89)ccc6ccc3c4c65)cc2)cc1] 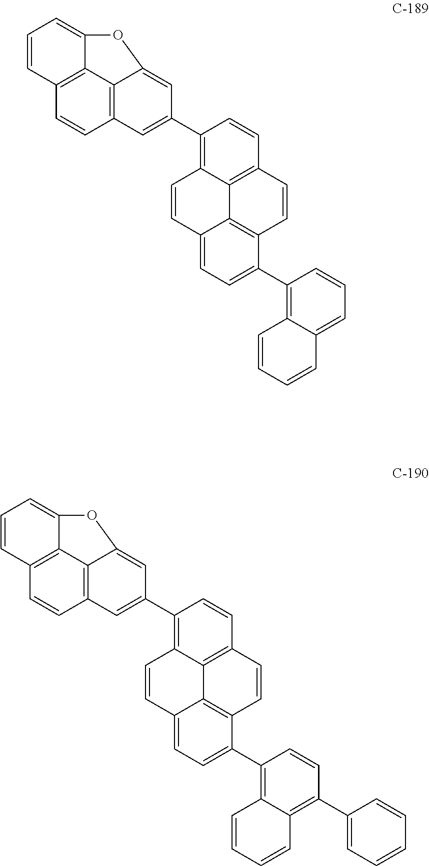 [SMILES: c1ccc2c(-c3ccc4ccc5c(-c6cc7ccc8cccc9oc(c6)c7c89)ccc6ccc3c4c65)cccc2c1 ; c1ccc(-c2ccc(-c3ccc4ccc5c(-c6cc7ccc8cccc9oc(c6)c7c89)ccc6ccc3c4c65)c3ccccc23)cc1] 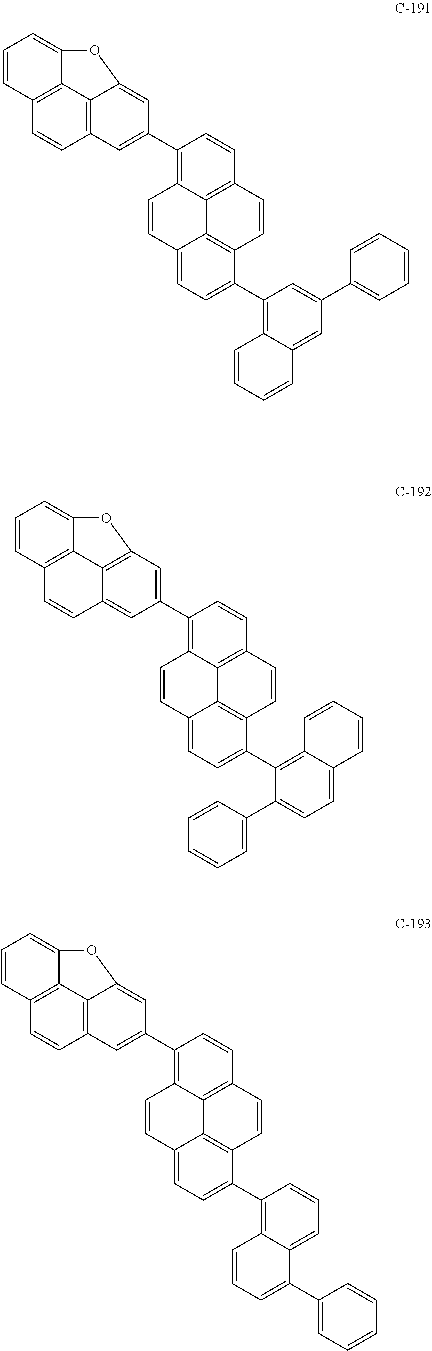 [SMILES: c1ccc(-c2cc(-c3ccc4ccc5c(-c6cc7ccc8cccc9oc(c6)c7c89)ccc6ccc3c4c65)c3ccccc3c2)cc1 ; c1ccc(-c2ccc3ccccc3c2-c2ccc3ccc4c(-c5cc6ccc7cccc8oc(c5)c6c78)ccc5ccc2c3c54)cc1 ; c1ccc(-c2cccc3c(-c4ccc5ccc6c(-c7cc8ccc9cccc%10oc(c7)c8c9%10)ccc7ccc4c5c76)cccc23)cc1] 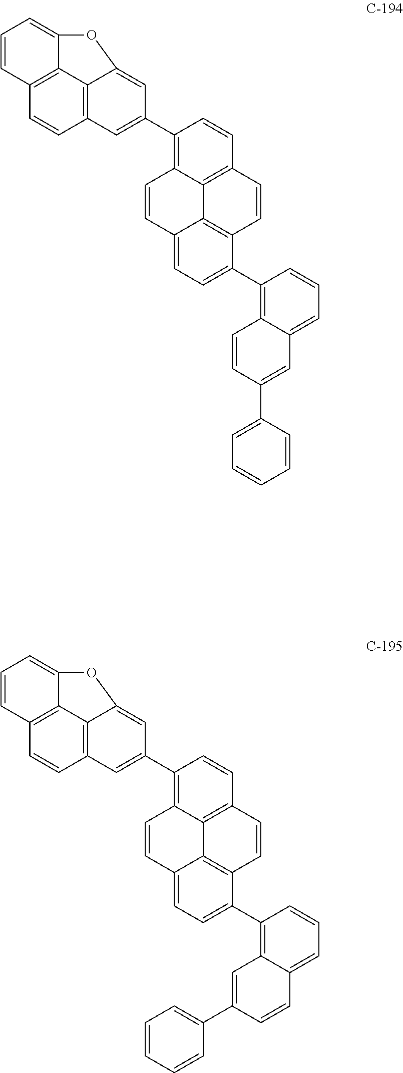 [SMILES: c1ccc(-c2ccc3c(-c4ccc5ccc6c(-c7cc8ccc9cccc%10oc(c7)c8c9%10)ccc7ccc4c5c76)cccc3c2)cc1 ; c1ccc(-c2ccc3cccc(-c4ccc5ccc6c(-c7cc8ccc9cccc%10oc(c7)c8c9%10)ccc7ccc4c5c76)c3c2)cc1] 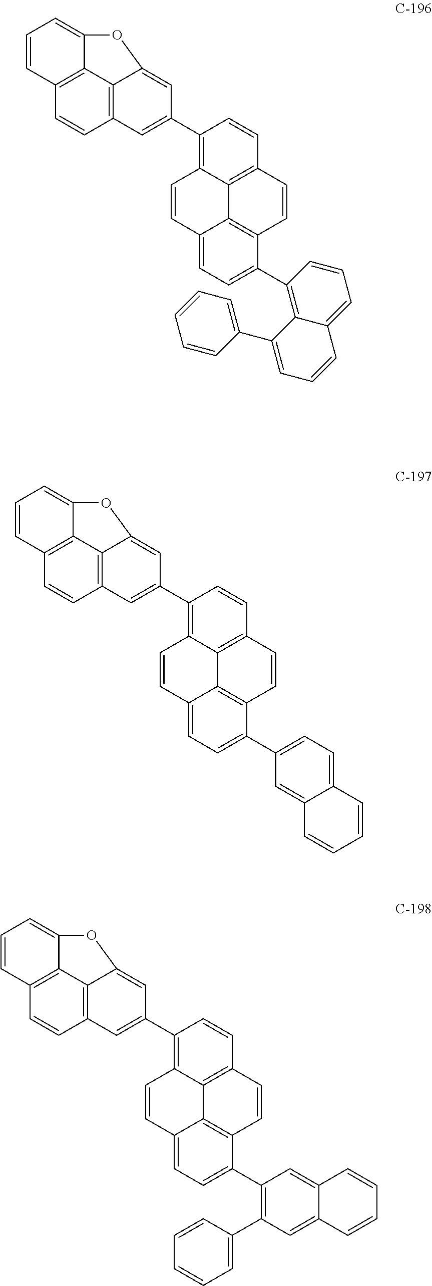 [SMILES: c1ccc(-c2cccc3cccc(-c4ccc5ccc6c(-c7cc8ccc9cccc%10oc(c7)c8c9%10)ccc7ccc4c5c76)c23)cc1 ; c1ccc2cc(-c3ccc4ccc5c(-c6cc7ccc8cccc9oc(c6)c7c89)ccc6ccc3c4c65)ccc2c1 ; c1ccc(-c2cc3ccccc3cc2-c2ccc3ccc4c(-c5cc6ccc7cccc8oc(c5)c6c78)ccc5ccc2c3c54)cc1] 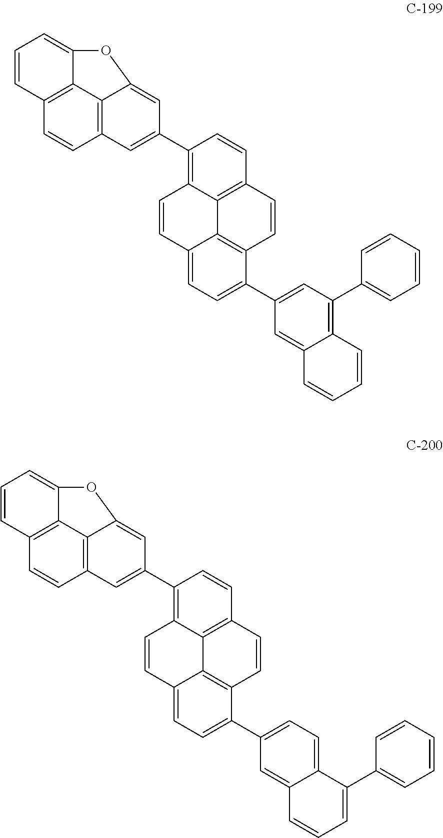 [SMILES: c1ccc(-c2cc(-c3ccc4ccc5c(-c6cc7ccc8cccc9oc(c6)c7c89)ccc6ccc3c4c65)cc3ccccc23)cc1 ; c1ccc(-c2cccc3cc(-c4ccc5ccc6c(-c7cc8ccc9cccc%10oc(c7)c8c9%10)ccc7ccc4c5c76)ccc23)cc1] 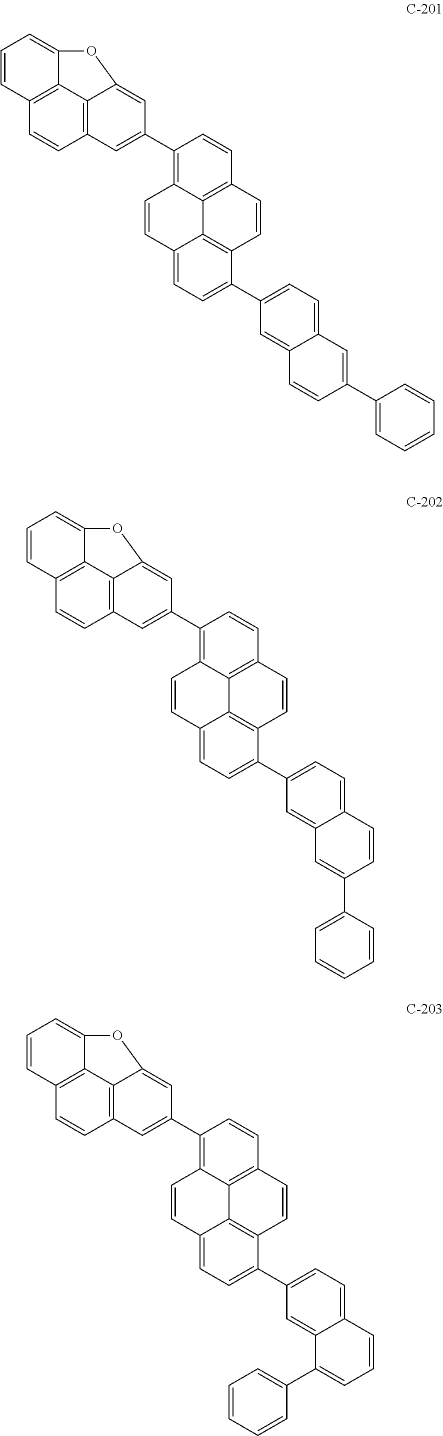 [SMILES: c1ccc(-c2ccc3cc(-c4ccc5ccc6c(-c7cc8ccc9cccc%10oc(c7)c8c9%10)ccc7ccc4c5c76)ccc3c2)cc1 ; c1ccc(-c2ccc3ccc(-c4ccc5ccc6c(-c7cc8ccc9cccc%10oc(c7)c8c9%10)ccc7ccc4c5c76)cc3c2)cc1 ; c1ccc(-c2cccc3ccc(-c4ccc5ccc6c(-c7cc8ccc9cccc%10oc(c7)c8c9%10)ccc7ccc4c5c76)cc23)cc1] 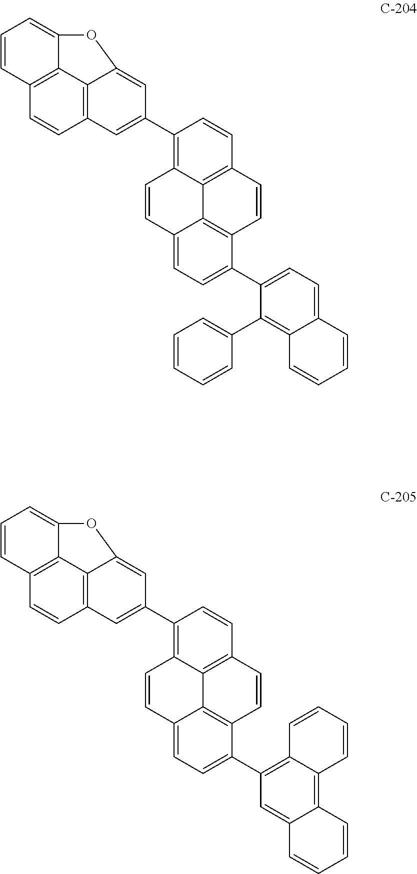 [SMILES: c1ccc(-c2c(-c3ccc4ccc5c(-c6cc7ccc8cccc9oc(c6)c7c89)ccc6ccc3c4c65)ccc3ccccc23)cc1 ; c1ccc2c(c1)cc(-c1ccc3ccc4c(-c5cc6ccc7cccc8oc(c5)c6c78)ccc5ccc1c3c54)c1ccccc12] 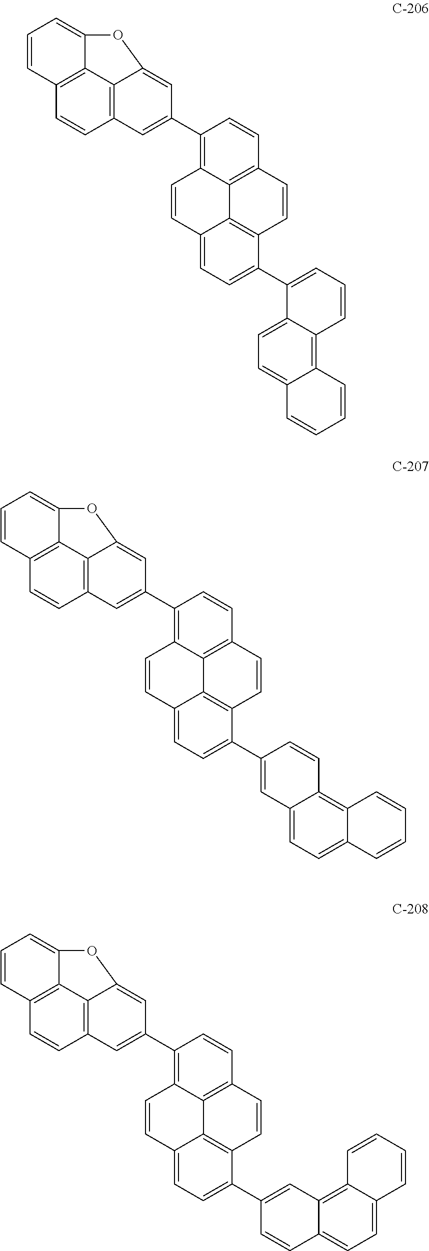 [SMILES: c1ccc2c(c1)ccc1c(-c3ccc4ccc5c(-c6cc7ccc8cccc9oc(c6)c7c89)ccc6ccc3c4c65)cccc12 ; c1ccc2c(c1)ccc1cc(-c3ccc4ccc5c(-c6cc7ccc8cccc9oc(c6)c7c89)ccc6ccc3c4c65)ccc12 ; c1ccc2c(c1)ccc1ccc(-c3ccc4ccc5c(-c6cc7ccc8cccc9oc(c6)c7c89)ccc6ccc3c4c65)cc12] 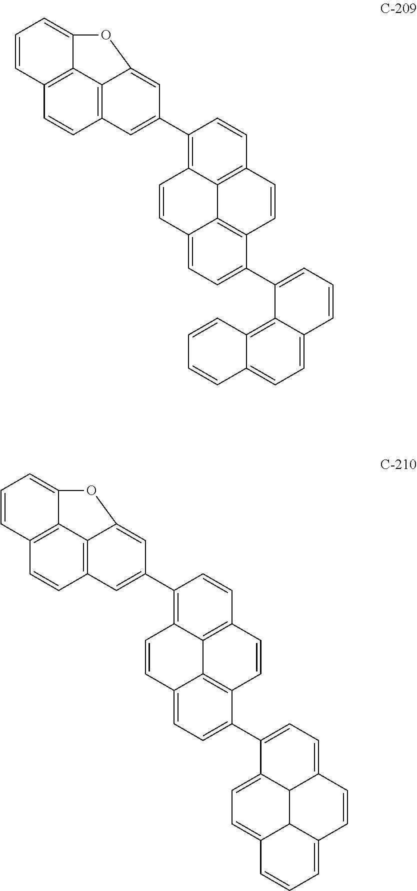 [SMILES: c1ccc2c(c1)ccc1cccc(-c3ccc4ccc5c(-c6cc7ccc8cccc9oc(c6)c7c89)ccc6ccc3c4c65)c12 ; C1=CC2=CC=C3C(c4ccc5ccc6c(-c7cc8ccc9cccc%10oc(c7)c8c9%10)ccc7ccc4c5c76)=CC=C4C=CC(=C1)C2C43] 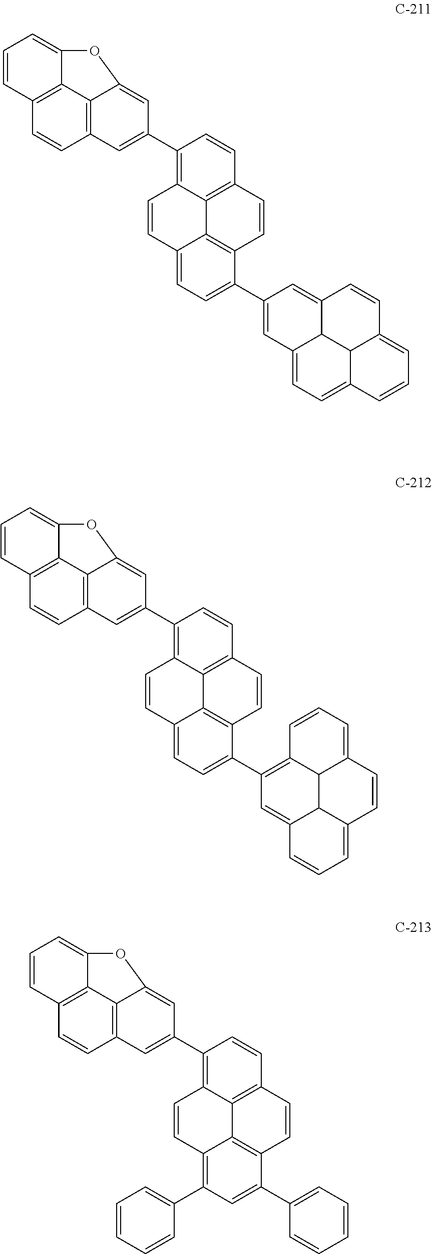 [SMILES: C1=CC2=CC=C3C=C(c4ccc5ccc6c(-c7cc8ccc9cccc%10oc(c7)c8c9%10)ccc7ccc4c5c76)C=C4C=CC(=C1)C2C34 ; C1=CC2=CC(c3ccc4ccc5c(-c6cc7ccc8cccc9oc(c6)c7c89)ccc6ccc3c4c65)=C3C=CC=C4C=CC(=C1)C2C43 ; c1ccc(-c2cc(-c3ccccc3)c3ccc4c(-c5cc6ccc7cccc8oc(c5)c6c78)ccc5ccc2c3c54)cc1] 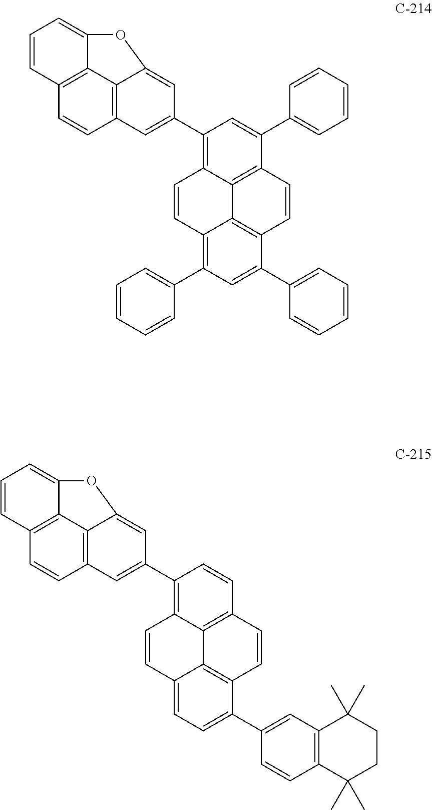 [SMILES: c1ccc(-c2cc(-c3ccccc3)c3ccc4c(-c5cc6ccc7cccc8oc(c5)c6c78)cc(-c5ccccc5)c5ccc2c3c54)cc1 ; CC1(C)CCC(C)(C)c2cc(-c3ccc4ccc5c(-c6cc7ccc8cccc9oc(c6)c7c89)ccc6ccc3c4c65)ccc21] 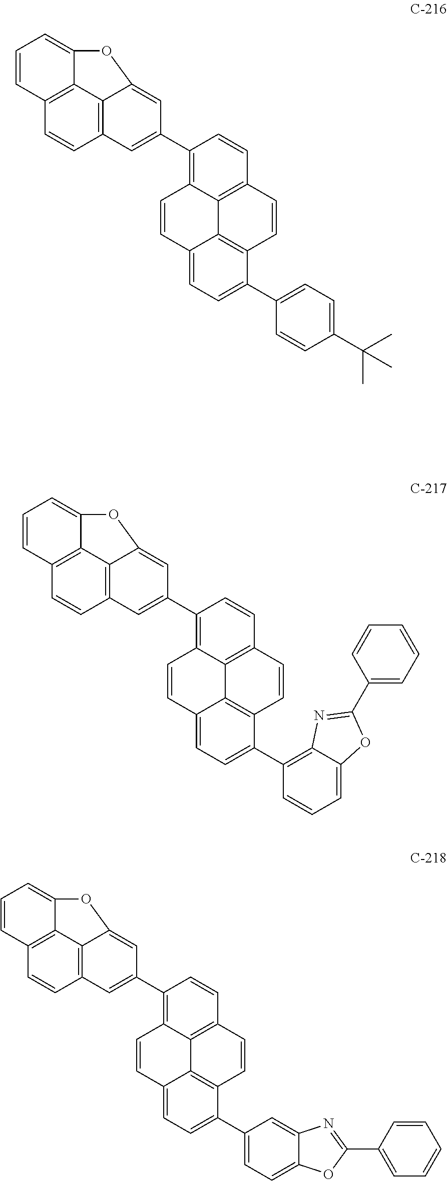 [SMILES: CC(C)(C)c1ccc(-c2ccc3ccc4c(-c5cc6ccc7cccc8oc(c5)c6c78)ccc5ccc2c3c54)cc1 ; c1ccc(-c2nc3c(-c4ccc5ccc6c(-c7cc8ccc9cccc%10oc(c7)c8c9%10)ccc7ccc4c5c76)cccc3o2)cc1 ; c1ccc(-c2nc3cc(-c4ccc5ccc6c(-c7cc8ccc9cccc%10oc(c7)c8c9%10)ccc7ccc4c5c76)ccc3o2)cc1] 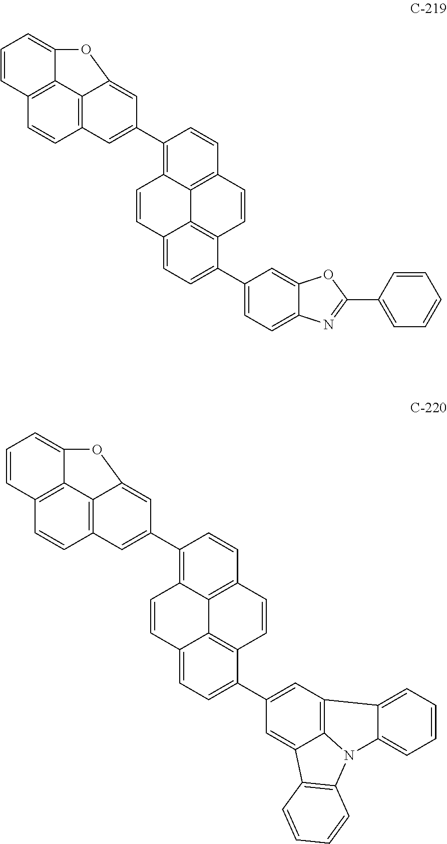 [SMILES: c1ccc(-c2nc3ccc(-c4ccc5ccc6c(-c7cc8ccc9cccc%10oc(c7)c8c9%10)ccc7ccc4c5c76)cc3o2)cc1 ; c1cc2ccc3cc(-c4ccc5ccc6c(-c7cc8c9ccccc9n9c%10ccccc%10c(c7)c89)ccc7ccc4c5c76)cc4oc(c1)c2c34] 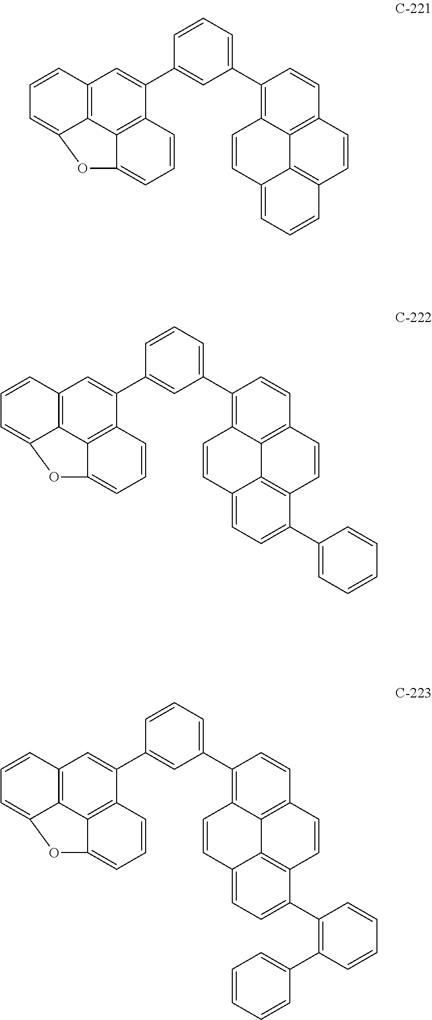 [SMILES: c1cc(-c2ccc3ccc4cccc5ccc2c3c45)cc(-c2cc3cccc4oc5cccc2c5c34)c1 ; c1ccc(-c2ccc3ccc4c(-c5cccc(-c6cc7cccc8oc9cccc6c9c78)c5)ccc5ccc2c3c54)cc1 ; c1ccc(-c2ccccc2-c2ccc3ccc4c(-c5cccc(-c6cc7cccc8oc9cccc6c9c78)c5)ccc5ccc2c3c54)cc1] 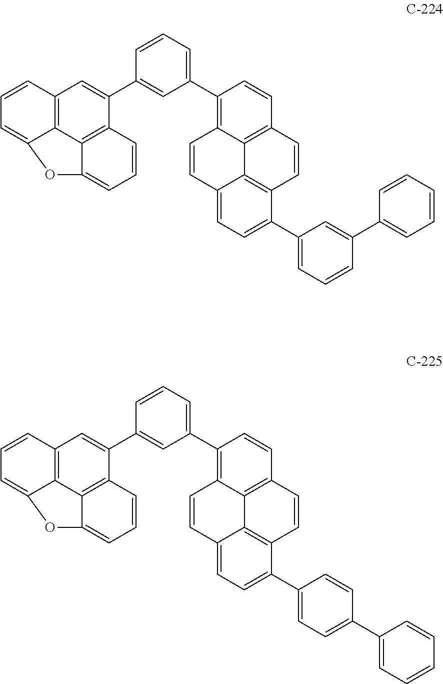 [SMILES: c1ccc(-c2cccc(-c3ccc4ccc5c(-c6cccc(-c7cc8cccc9oc%10cccc7c%10c89)c6)ccc6ccc3c4c65)c2)cc1 ; c1ccc(-c2ccc(-c3ccc4ccc5c(-c6cccc(-c7cc8cccc9oc%10cccc7c%10c89)c6)ccc6ccc3c4c65)cc2)cc1] 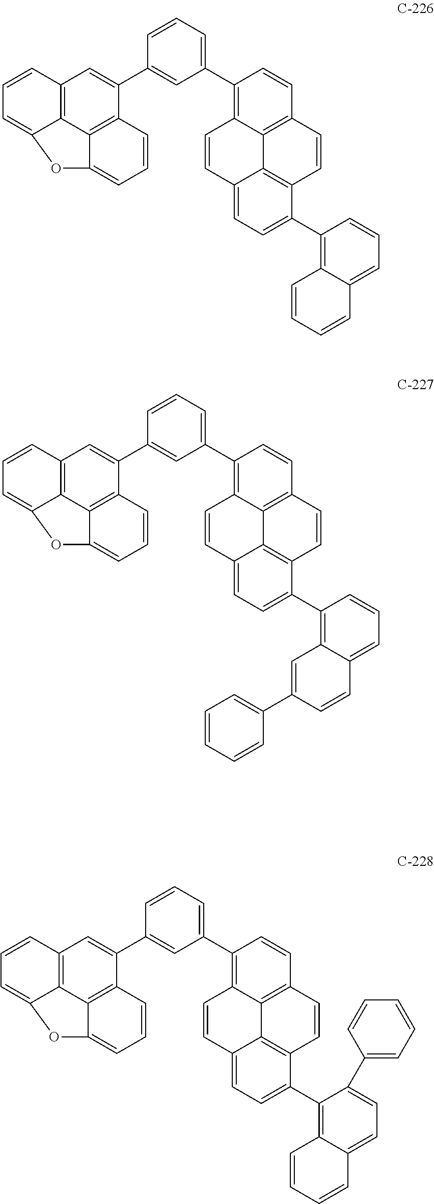 [SMILES: c1cc(-c2ccc3ccc4c(-c5cccc6ccccc56)ccc5ccc2c3c54)cc(-c2cc3cccc4oc5cccc2c5c34)c1 ; c1ccc(-c2ccc3cccc(-c4ccc5ccc6c(-c7cccc(-c8cc9cccc%10oc%11cccc8c%11c9%10)c7)ccc7ccc4c5c76)c3c2)cc1 ; c1ccc(-c2ccc3ccccc3c2-c2ccc3ccc4c(-c5cccc(-c6cc7cccc8oc9cccc6c9c78)c5)ccc5ccc2c3c54)cc1] 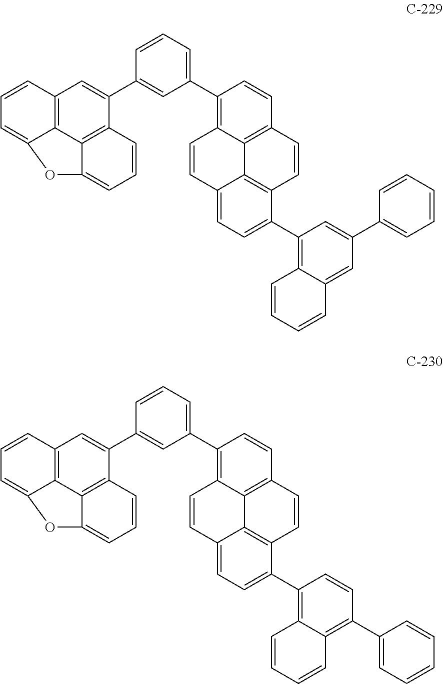 [SMILES: c1ccc(-c2cc(-c3ccc4ccc5c(-c6cccc(-c7cc8cccc9oc%10cccc7c%10c89)c6)ccc6ccc3c4c65)c3ccccc3c2)cc1 ; c1ccc(-c2ccc(-c3ccc4ccc5c(-c6cccc(-c7cc8cccc9oc%10cccc7c%10c89)c6)ccc6ccc3c4c65)c3ccccc23)cc1] 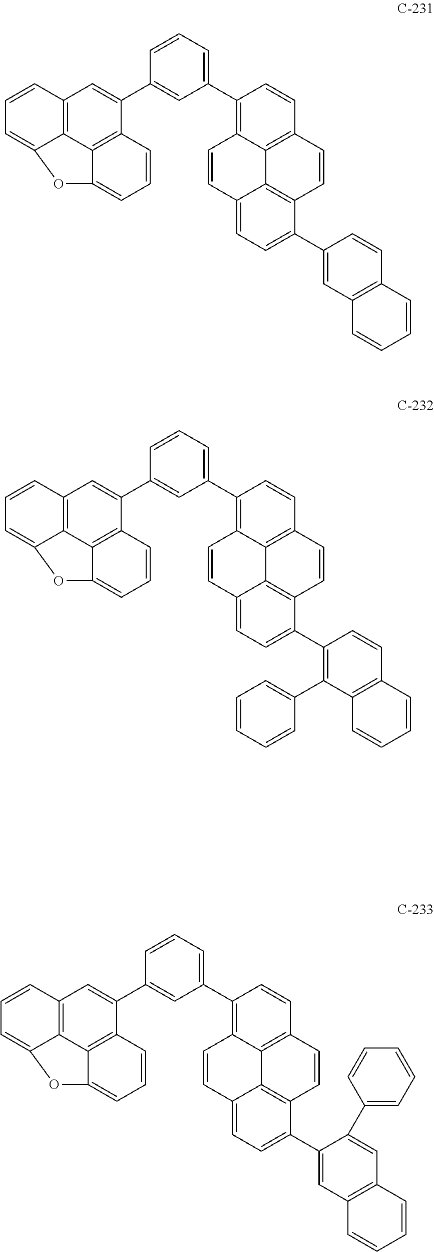 [SMILES: c1cc(-c2ccc3ccc4c(-c5ccc6ccccc6c5)ccc5ccc2c3c54)cc(-c2cc3cccc4oc5cccc2c5c34)c1 ; c1ccc(-c2c(-c3ccc4ccc5c(-c6cccc(-c7cc8cccc9oc%10cccc7c%10c89)c6)ccc6ccc3c4c65)ccc3ccccc23)cc1 ; c1ccc(-c2cc3ccccc3cc2-c2ccc3ccc4c(-c5cccc(-c6cc7cccc8oc9cccc6c9c78)c5)ccc5ccc2c3c54)cc1] 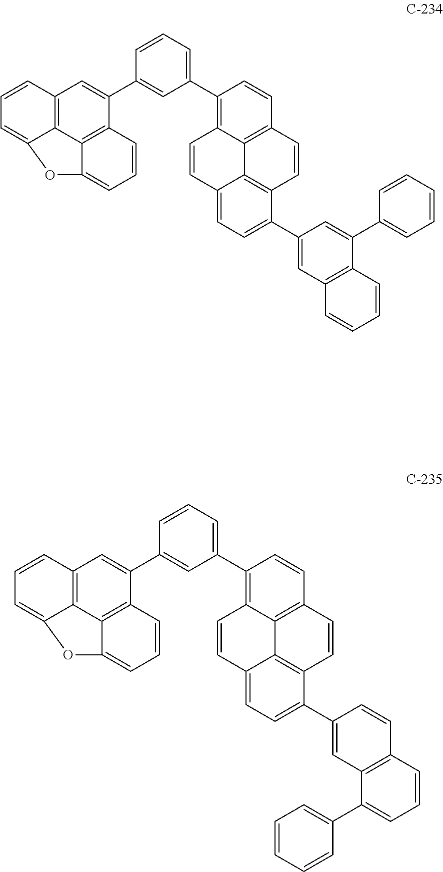 [SMILES: c1ccc(-c2cc(-c3ccc4ccc5c(-c6cccc(-c7cc8cccc9oc%10cccc7c%10c89)c6)ccc6ccc3c4c65)cc3ccccc23)cc1 ; c1ccc(-c2cccc3ccc(-c4ccc5ccc6c(-c7cccc(-c8cc9cccc%10oc%11cccc8c%11c9%10)c7)ccc7ccc4c5c76)cc23)cc1] 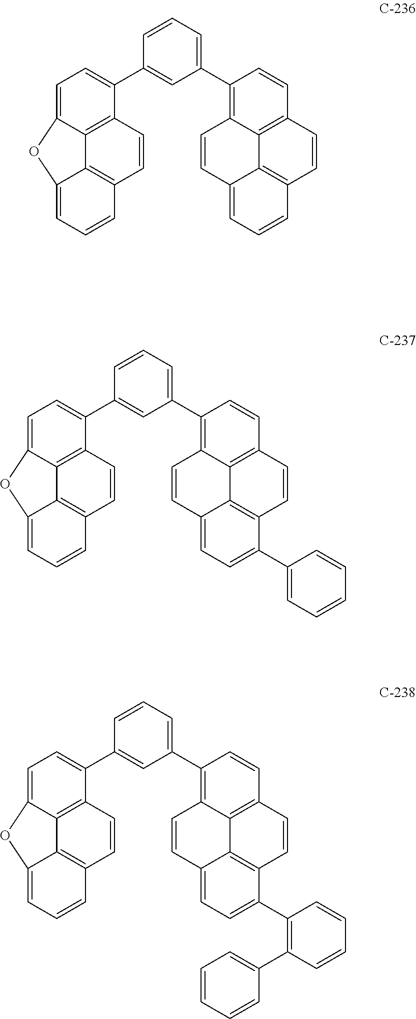 [SMILES: c1cc(-c2ccc3ccc4cccc5ccc2c3c45)cc(-c2ccc3oc4cccc5ccc2c3c54)c1 ; c1ccc(-c2ccc3ccc4c(-c5cccc(-c6ccc7oc8cccc9ccc6c7c98)c5)ccc5ccc2c3c54)cc1 ; c1ccc(-c2ccccc2-c2ccc3ccc4c(-c5cccc(-c6ccc7oc8cccc9ccc6c7c98)c5)ccc5ccc2c3c54)cc1] 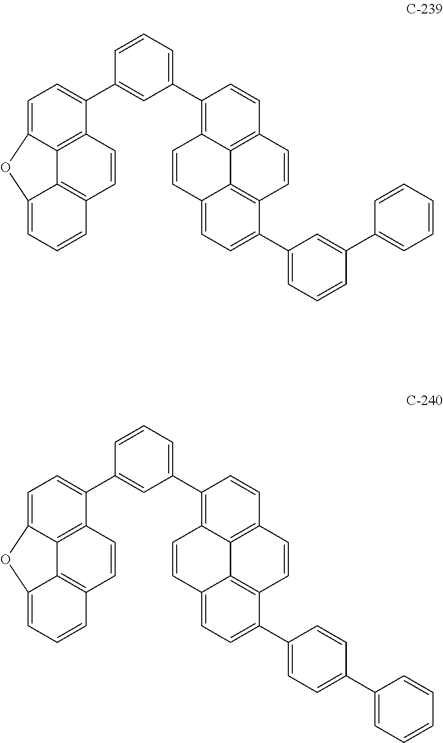 [SMILES: c1ccc(-c2cccc(-c3ccc4ccc5c(-c6cccc(-c7ccc8oc9cccc%10ccc7c8c%109)c6)ccc6ccc3c4c65)c2)cc1 ; c1ccc(-c2ccc(-c3ccc4ccc5c(-c6cccc(-c7ccc8oc9cccc%10ccc7c8c%109)c6)ccc6ccc3c4c65)cc2)cc1] 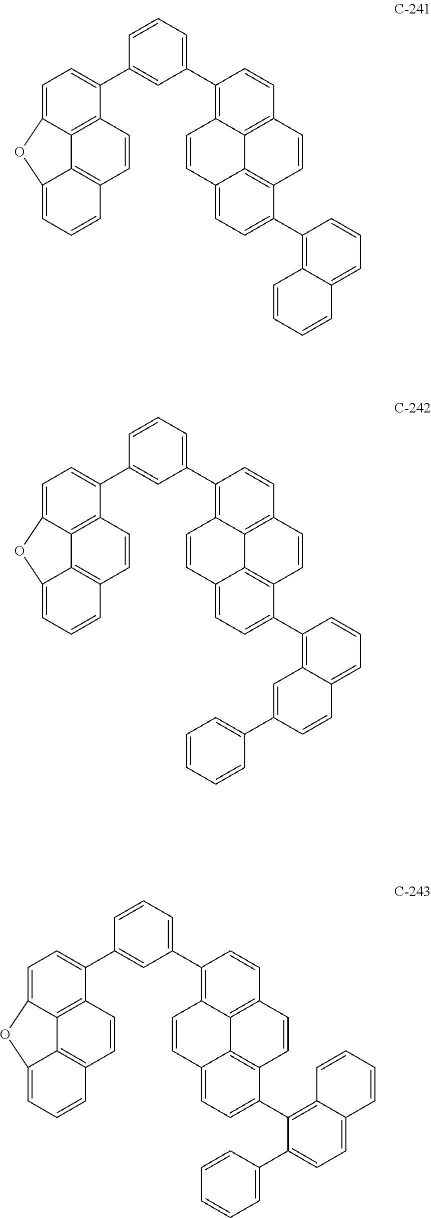 [SMILES: c1cc(-c2ccc3ccc4c(-c5cccc6ccccc56)ccc5ccc2c3c54)cc(-c2ccc3oc4cccc5ccc2c3c54)c1 ; c1ccc(-c2ccc3cccc(-c4ccc5ccc6c(-c7cccc(-c8ccc9oc%10cccc%11ccc8c9c%11%10)c7)ccc7ccc4c5c76)c3c2)cc1 ; c1ccc(-c2ccc3ccccc3c2-c2ccc3ccc4c(-c5cccc(-c6ccc7oc8cccc9ccc6c7c98)c5)ccc5ccc2c3c54)cc1] 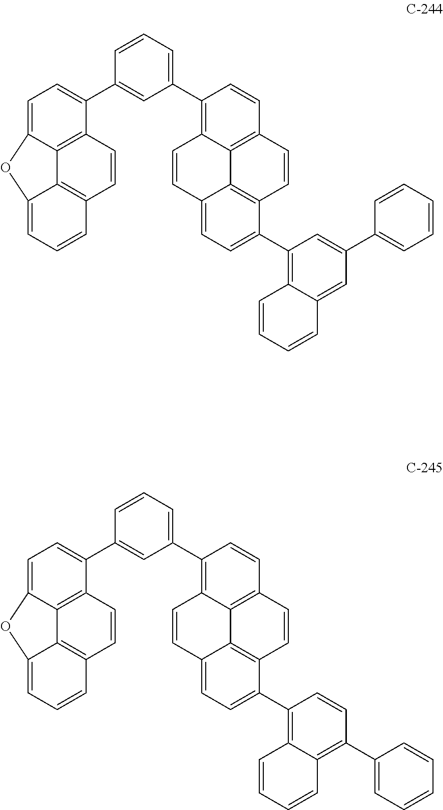 [SMILES: c1ccc(-c2cc(-c3ccc4ccc5c(-c6cccc(-c7ccc8oc9cccc%10ccc7c8c%109)c6)ccc6ccc3c4c65)c3ccccc3c2)cc1 ; c1ccc(-c2ccc(-c3ccc4ccc5c(-c6cccc(-c7ccc8oc9cccc%10ccc7c8c%109)c6)ccc6ccc3c4c65)c3ccccc23)cc1] 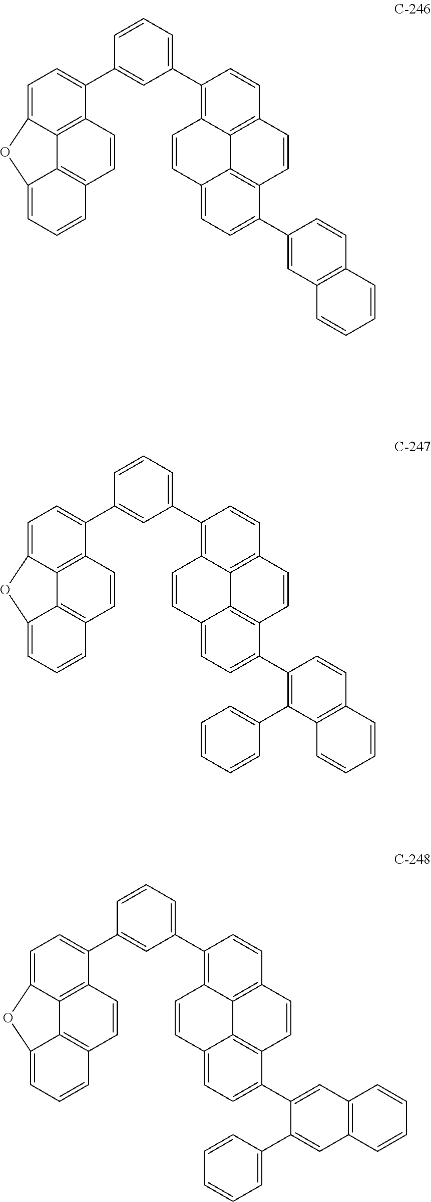 [SMILES: c1cc(-c2ccc3ccc4c(-c5ccc6ccccc6c5)ccc5ccc2c3c54)cc(-c2ccc3oc4cccc5ccc2c3c54)c1 ; c1ccc(-c2c(-c3ccc4ccc5c(-c6cccc(-c7ccc8oc9cccc%10ccc7c8c%109)c6)ccc6ccc3c4c65)ccc3ccccc23)cc1 ; c1ccc(-c2cc3ccccc3cc2-c2ccc3ccc4c(-c5cccc(-c6ccc7oc8cccc9ccc6c7c98)c5)ccc5ccc2c3c54)cc1] 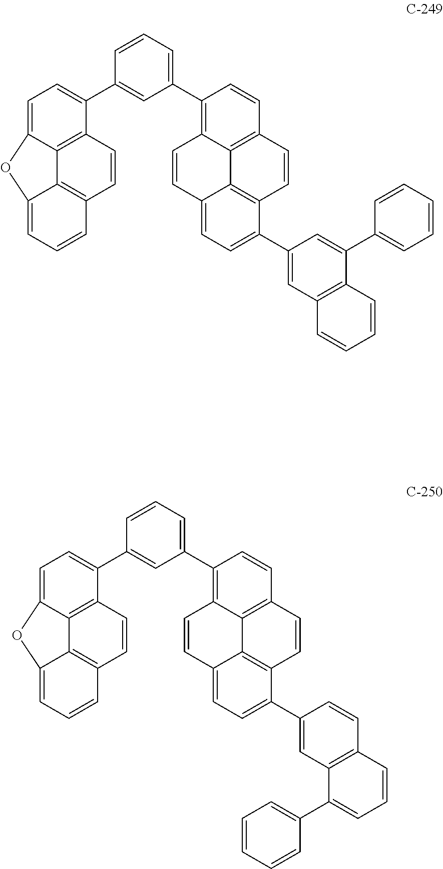 [SMILES: c1ccc(-c2cc(-c3ccc4ccc5c(-c6cccc(-c7ccc8oc9cccc%10ccc7c8c%109)c6)ccc6ccc3c4c65)cc3ccccc23)cc1 ; c1ccc(-c2cccc3ccc(-c4ccc5ccc6c(-c7cccc(-c8ccc9oc%10cccc%11ccc8c9c%11%10)c7)ccc7ccc4c5c76)cc23)cc1] 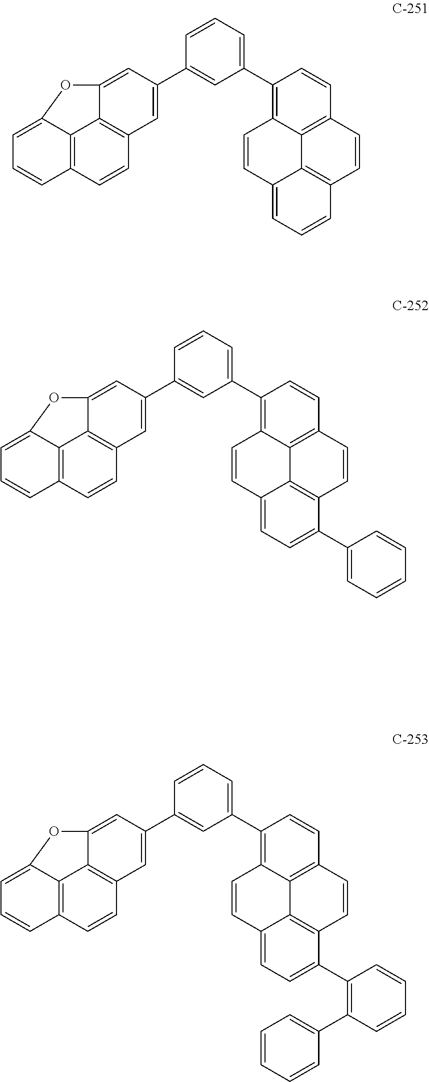 [SMILES: c1cc(-c2cc3ccc4cccc5oc(c2)c3c45)cc(-c2ccc3ccc4cccc5ccc2c3c45)c1 ; c1ccc(-c2ccc3ccc4c(-c5cccc(-c6cc7ccc8cccc9oc(c6)c7c89)c5)ccc5ccc2c3c54)cc1 ; c1ccc(-c2ccccc2-c2ccc3ccc4c(-c5cccc(-c6cc7ccc8cccc9oc(c6)c7c89)c5)ccc5ccc2c3c54)cc1] 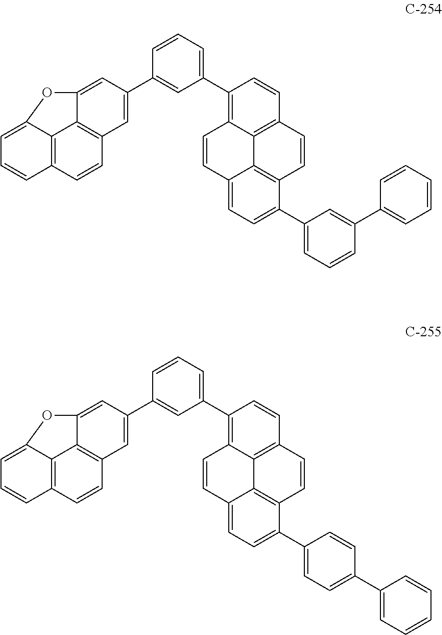 [SMILES: c1ccc(-c2cccc(-c3ccc4ccc5c(-c6cccc(-c7cc8ccc9cccc%10oc(c7)c8c9%10)c6)ccc6ccc3c4c65)c2)cc1 ; c1ccc(-c2ccc(-c3ccc4ccc5c(-c6cccc(-c7cc8ccc9cccc%10oc(c7)c8c9%10)c6)ccc6ccc3c4c65)cc2)cc1] 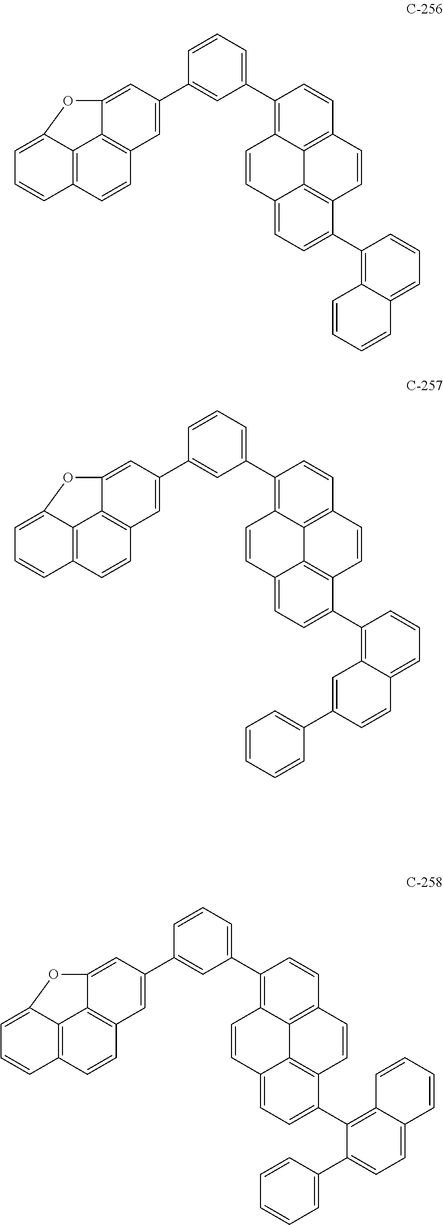 [SMILES: c1cc(-c2cc3ccc4cccc5oc(c2)c3c45)cc(-c2ccc3ccc4c(-c5cccc6ccccc56)ccc5ccc2c3c54)c1 ; c1ccc(-c2ccc3cccc(-c4ccc5ccc6c(-c7cccc(-c8cc9ccc%10cccc%11oc(c8)c9c%10%11)c7)ccc7ccc4c5c76)c3c2)cc1 ; c1ccc(-c2ccc3ccccc3c2-c2ccc3ccc4c(-c5cccc(-c6cc7ccc8cccc9oc(c6)c7c89)c5)ccc5ccc2c3c54)cc1] 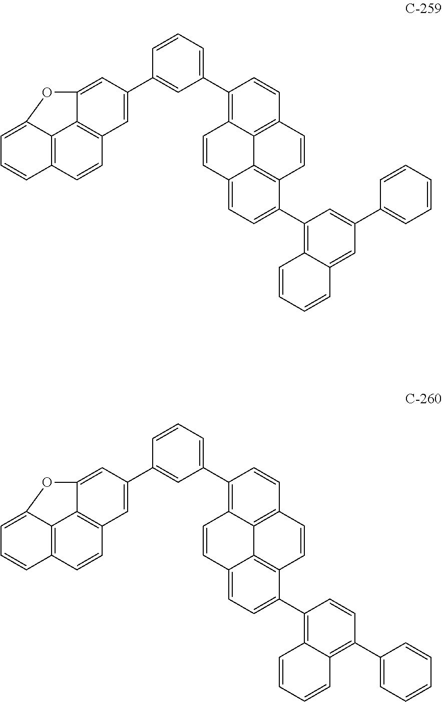 [SMILES: c1ccc(-c2cc(-c3ccc4ccc5c(-c6cccc(-c7cc8ccc9cccc%10oc(c7)c8c9%10)c6)ccc6ccc3c4c65)c3ccccc3c2)cc1 ; c1ccc(-c2ccc(-c3ccc4ccc5c(-c6cccc(-c7cc8ccc9cccc%10oc(c7)c8c9%10)c6)ccc6ccc3c4c65)c3ccccc23)cc1] 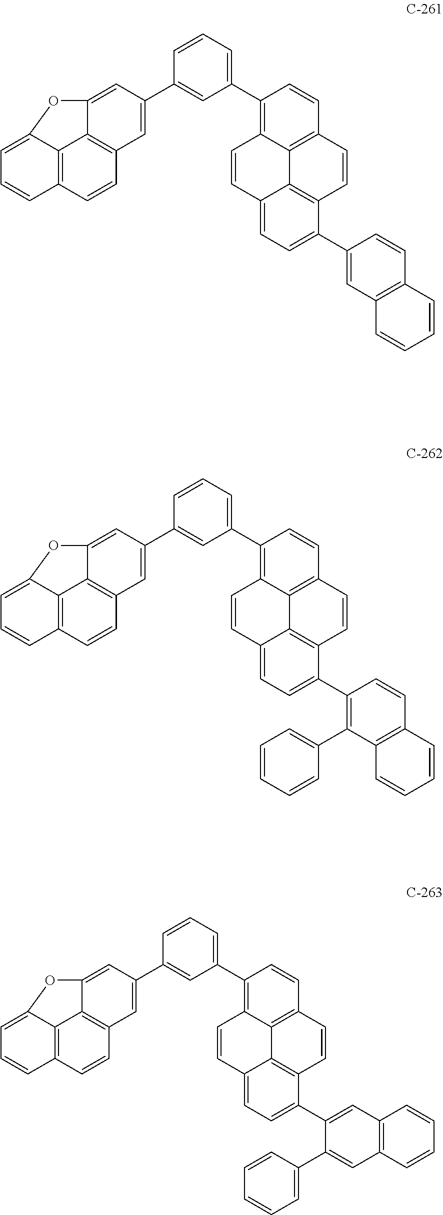 [SMILES: c1cc(-c2cc3ccc4cccc5oc(c2)c3c45)cc(-c2ccc3ccc4c(-c5ccc6ccccc6c5)ccc5ccc2c3c54)c1 ; c1ccc(-c2c(-c3ccc4ccc5c(-c6cccc(-c7cc8ccc9cccc%10oc(c7)c8c9%10)c6)ccc6ccc3c4c65)ccc3ccccc23)cc1 ; c1ccc(-c2cc3ccccc3cc2-c2ccc3ccc4c(-c5cccc(-c6cc7ccc8cccc9oc(c6)c7c89)c5)ccc5ccc2c3c54)cc1] 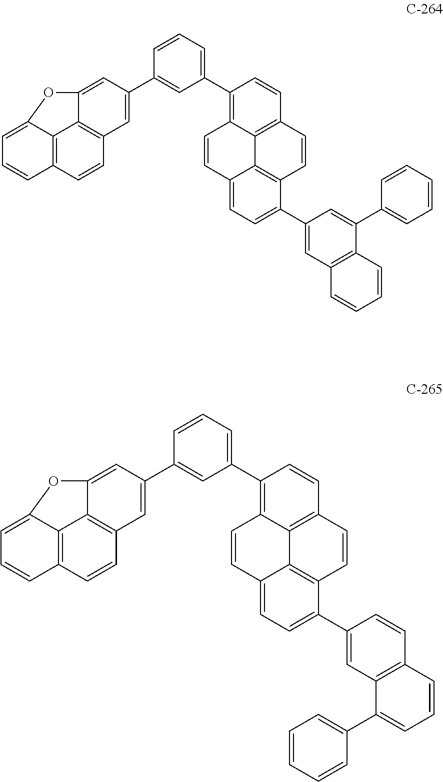 [SMILES: c1ccc(-c2cc(-c3ccc4ccc5c(-c6cccc(-c7cc8ccc9cccc%10oc(c7)c8c9%10)c6)ccc6ccc3c4c65)cc3ccccc23)cc1 ; c1ccc(-c2cccc3ccc(-c4ccc5ccc6c(-c7cccc(-c8cc9ccc%10cccc%11oc(c8)c9c%10%11)c7)ccc7ccc4c5c76)cc23)cc1] 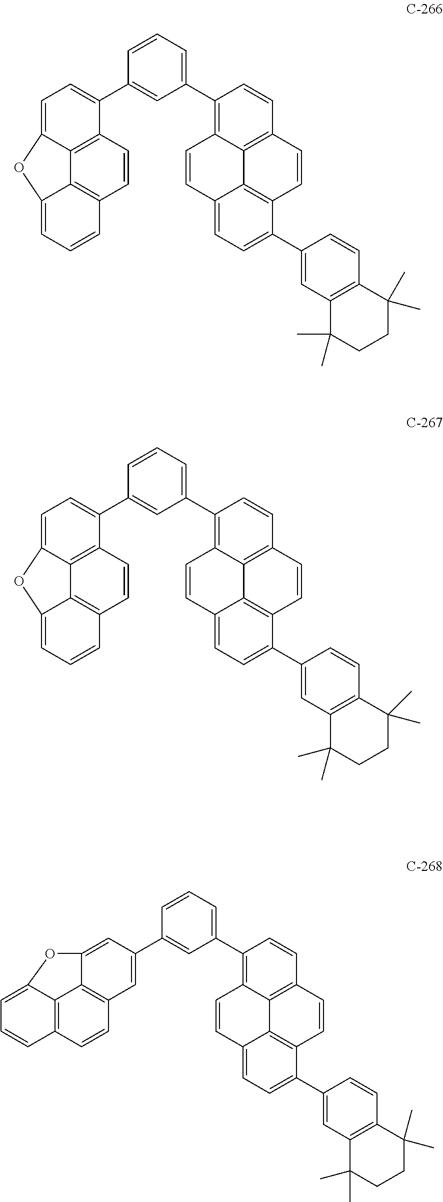 [SMILES: CC1(C)CCC(C)(C)c2cc(-c3ccc4ccc5c(-c6cccc(-c7ccc8oc9cccc%10ccc7c8c%109)c6)ccc6ccc3c4c65)ccc21 ; CC1(C)CCC(C)(C)c2cc(-c3ccc4ccc5c(-c6cccc(-c7ccc8oc9cccc%10ccc7c8c%109)c6)ccc6ccc3c4c65)ccc21 ; CC1(C)CCC(C)(C)c2cc(-c3ccc4ccc5c(-c6cccc(-c7cc8ccc9cccc%10oc(c7)c8c9%10)c6)ccc6ccc3c4c65)ccc21] 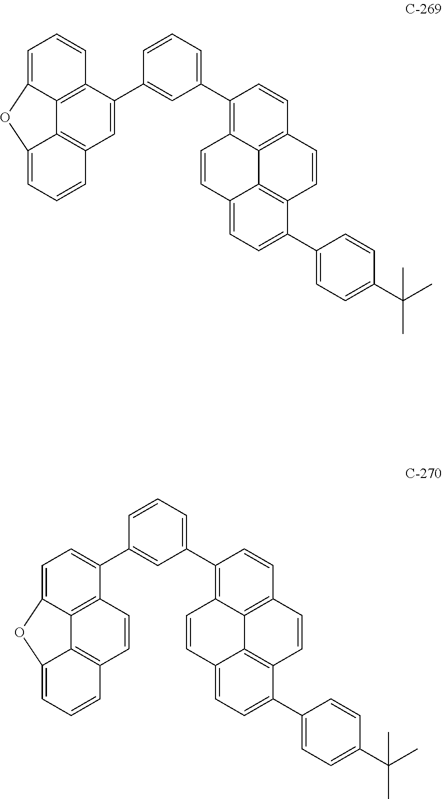 [SMILES: CC(C)(C)c1ccc(-c2ccc3ccc4c(-c5cccc(-c6cc7cccc8oc9cccc6c9c78)c5)ccc5ccc2c3c54)cc1 ; CC(C)(C)c1ccc(-c2ccc3ccc4c(-c5cccc(-c6ccc7oc8cccc9ccc6c7c98)c5)ccc5ccc2c3c54)cc1] 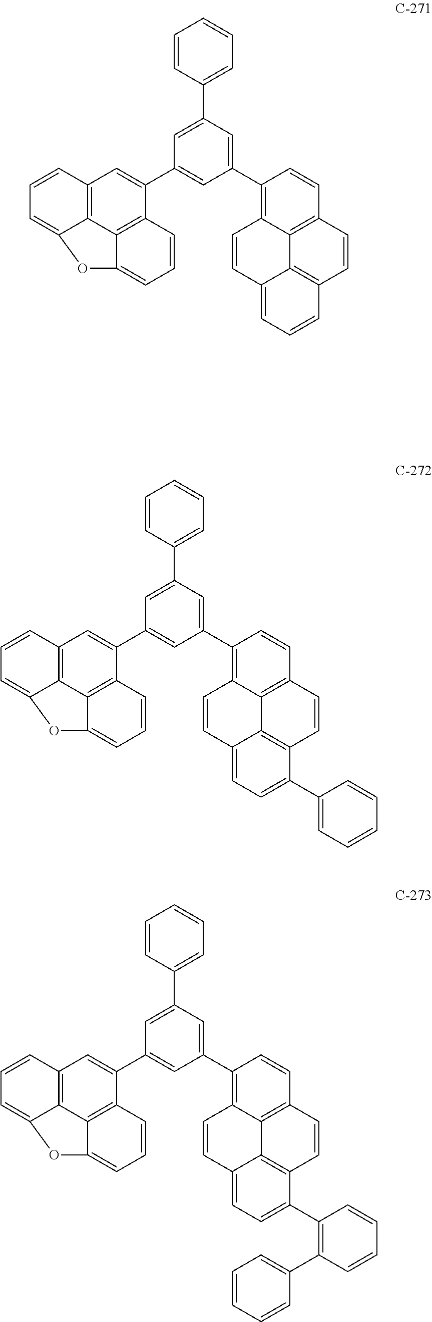 [SMILES: c1ccc(-c2cc(-c3ccc4ccc5cccc6ccc3c4c56)cc(-c3cc4cccc5oc6cccc3c6c45)c2)cc1 ; c1ccc(-c2cc(-c3ccc4ccc5c(-c6ccccc6)ccc6ccc3c4c65)cc(-c3cc4cccc5oc6cccc3c6c45)c2)cc1 ; c1ccc(-c2cc(-c3ccc4ccc5c(-c6ccccc6-c6ccccc6)ccc6ccc3c4c65)cc(-c3cc4cccc5oc6cccc3c6c45)c2)cc1] 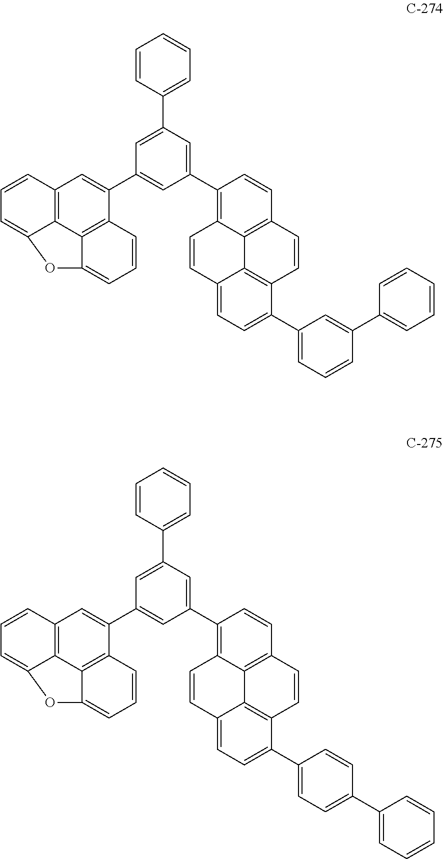 [SMILES: c1ccc(-c2cccc(-c3ccc4ccc5c(-c6cc(-c7ccccc7)cc(-c7cc8cccc9oc%10cccc7c%10c89)c6)ccc6ccc3c4c65)c2)cc1 ; c1ccc(-c2ccc(-c3ccc4ccc5c(-c6cc(-c7ccccc7)cc(-c7cc8cccc9oc%10cccc7c%10c89)c6)ccc6ccc3c4c65)cc2)cc1] 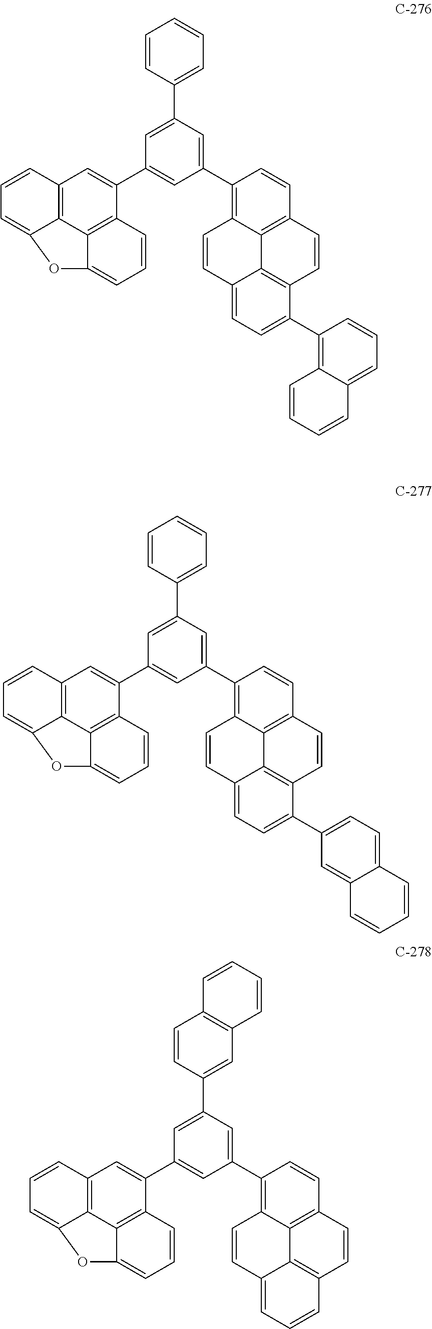 [SMILES: c1ccc(-c2cc(-c3ccc4ccc5c(-c6cccc7ccccc67)ccc6ccc3c4c65)cc(-c3cc4cccc5oc6cccc3c6c45)c2)cc1 ; c1ccc(-c2cc(-c3ccc4ccc5c(-c6ccc7ccccc7c6)ccc6ccc3c4c65)cc(-c3cc4cccc5oc6cccc3c6c45)c2)cc1 ; c1ccc2cc(-c3cc(-c4ccc5ccc6cccc7ccc4c5c67)cc(-c4cc5cccc6oc7cccc4c7c56)c3)ccc2c1] 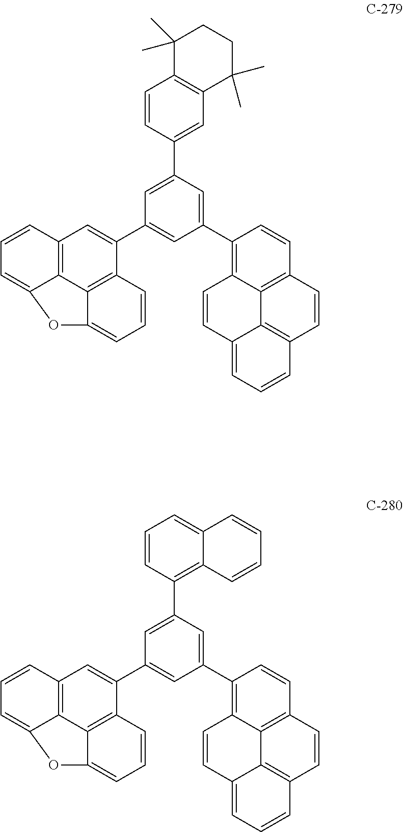 [SMILES: CC1(C)CCC(C)(C)c2cc(-c3cc(-c4ccc5ccc6cccc7ccc4c5c67)cc(-c4cc5cccc6oc7cccc4c7c56)c3)ccc21 ; c1ccc2c(-c3cc(-c4ccc5ccc6cccc7ccc4c5c67)cc(-c4cc5cccc6oc7cccc4c7c56)c3)cccc2c1] 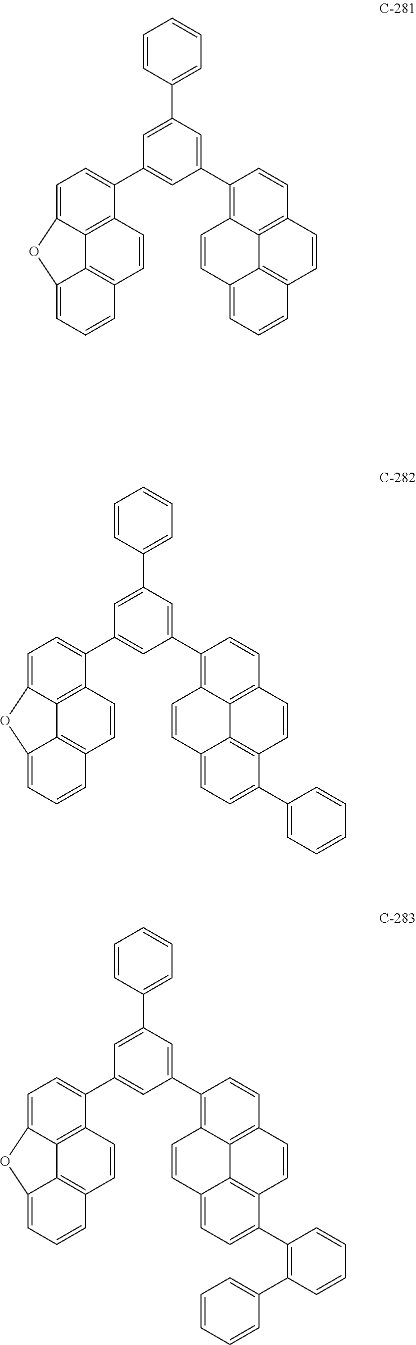 [SMILES: c1ccc(-c2cc(-c3ccc4ccc5cccc6ccc3c4c56)cc(-c3ccc4oc5cccc6ccc3c4c65)c2)cc1 ; c1ccc(-c2cc(-c3ccc4ccc5c(-c6ccccc6)ccc6ccc3c4c65)cc(-c3ccc4oc5cccc6ccc3c4c65)c2)cc1 ; c1ccc(-c2cc(-c3ccc4ccc5c(-c6ccccc6-c6ccccc6)ccc6ccc3c4c65)cc(-c3ccc4oc5cccc6ccc3c4c65)c2)cc1] 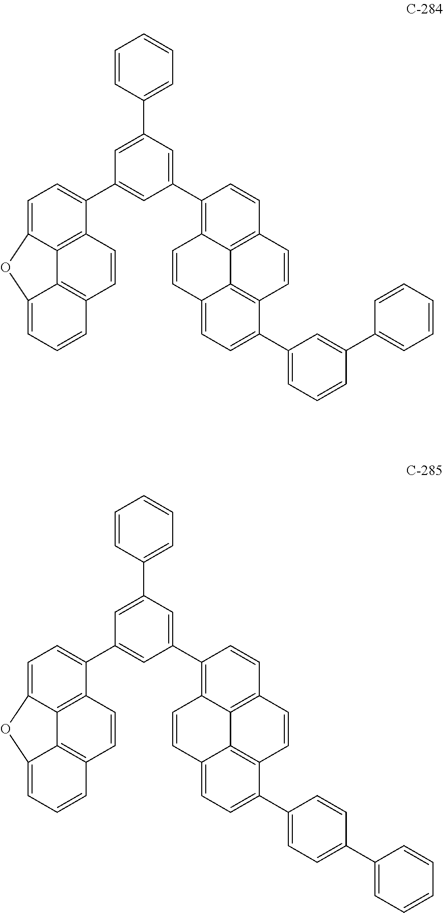 [SMILES: c1ccc(-c2cccc(-c3ccc4ccc5c(-c6cc(-c7ccccc7)cc(-c7ccc8oc9cccc%10ccc7c8c%109)c6)ccc6ccc3c4c65)c2)cc1 ; c1ccc(-c2ccc(-c3ccc4ccc5c(-c6cc(-c7ccccc7)cc(-c7ccc8oc9cccc%10ccc7c8c%109)c6)ccc6ccc3c4c65)cc2)cc1] 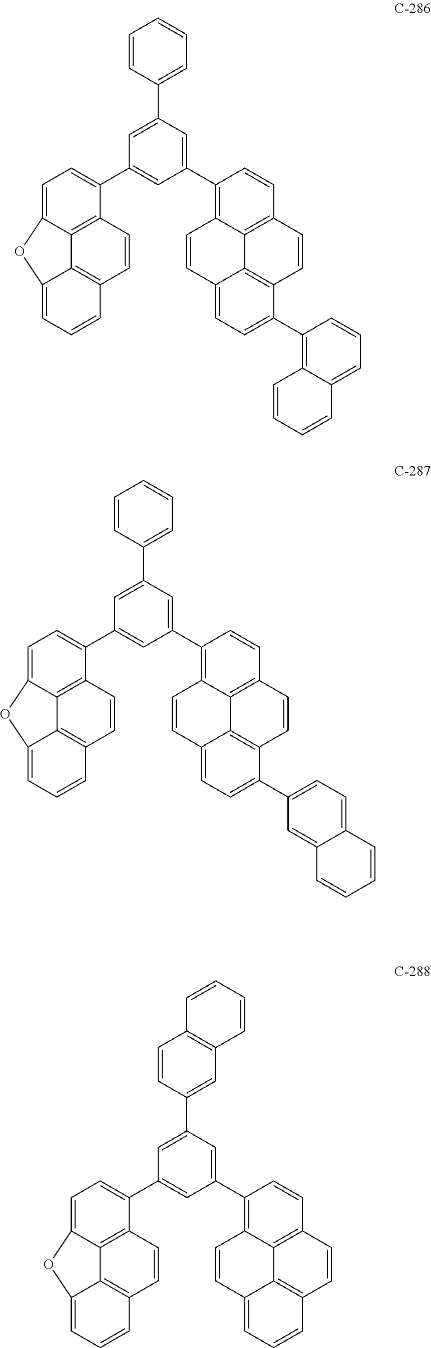 [SMILES: c1ccc(-c2cc(-c3ccc4ccc5c(-c6cccc7ccccc67)ccc6ccc3c4c65)cc(-c3ccc4oc5cccc6ccc3c4c65)c2)cc1 ; c1ccc(-c2cc(-c3ccc4ccc5c(-c6ccc7ccccc7c6)ccc6ccc3c4c65)cc(-c3ccc4oc5cccc6ccc3c4c65)c2)cc1 ; c1ccc2cc(-c3cc(-c4ccc5ccc6cccc7ccc4c5c67)cc(-c4ccc5oc6cccc7ccc4c5c76)c3)ccc2c1] 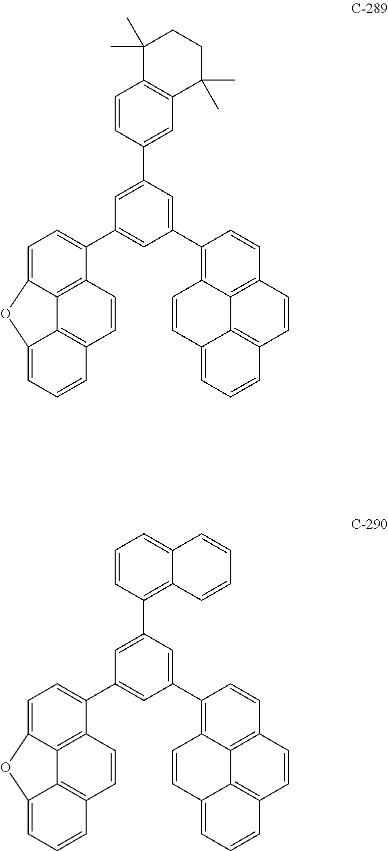 [SMILES: CC1(C)CCC(C)(C)c2cc(-c3cc(-c4ccc5ccc6cccc7ccc4c5c67)cc(-c4ccc5oc6cccc7ccc4c5c76)c3)ccc21 ; c1ccc2c(-c3cc(-c4ccc5ccc6cccc7ccc4c5c67)cc(-c4ccc5oc6cccc7ccc4c5c76)c3)cccc2c1] 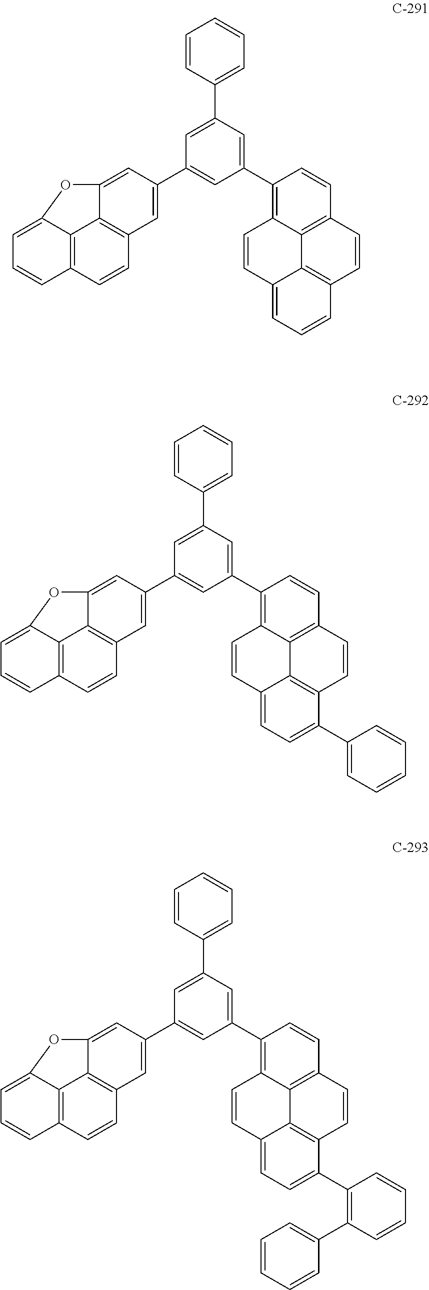 [SMILES: c1ccc(-c2cc(-c3cc4ccc5cccc6oc(c3)c4c56)cc(-c3ccc4ccc5cccc6ccc3c4c56)c2)cc1 ; c1ccc(-c2cc(-c3cc4ccc5cccc6oc(c3)c4c56)cc(-c3ccc4ccc5c(-c6ccccc6)ccc6ccc3c4c65)c2)cc1 ; c1ccc(-c2cc(-c3cc4ccc5cccc6oc(c3)c4c56)cc(-c3ccc4ccc5c(-c6ccccc6-c6ccccc6)ccc6ccc3c4c65)c2)cc1] 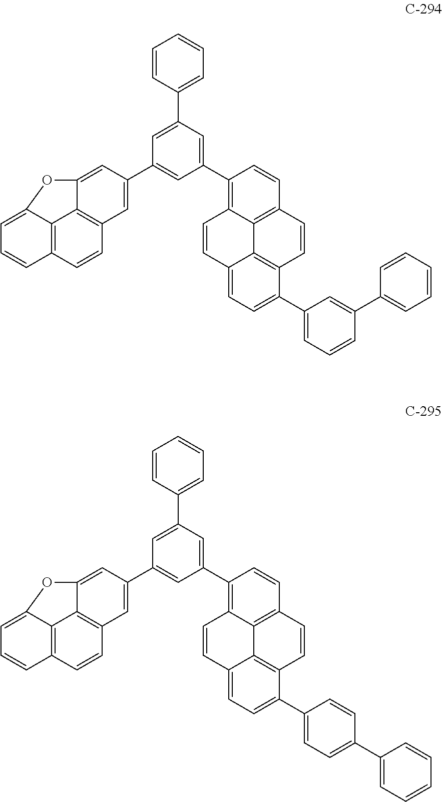 [SMILES: c1ccc(-c2cccc(-c3ccc4ccc5c(-c6cc(-c7ccccc7)cc(-c7cc8ccc9cccc%10oc(c7)c8c9%10)c6)ccc6ccc3c4c65)c2)cc1 ; c1ccc(-c2ccc(-c3ccc4ccc5c(-c6cc(-c7ccccc7)cc(-c7cc8ccc9cccc%10oc(c7)c8c9%10)c6)ccc6ccc3c4c65)cc2)cc1] 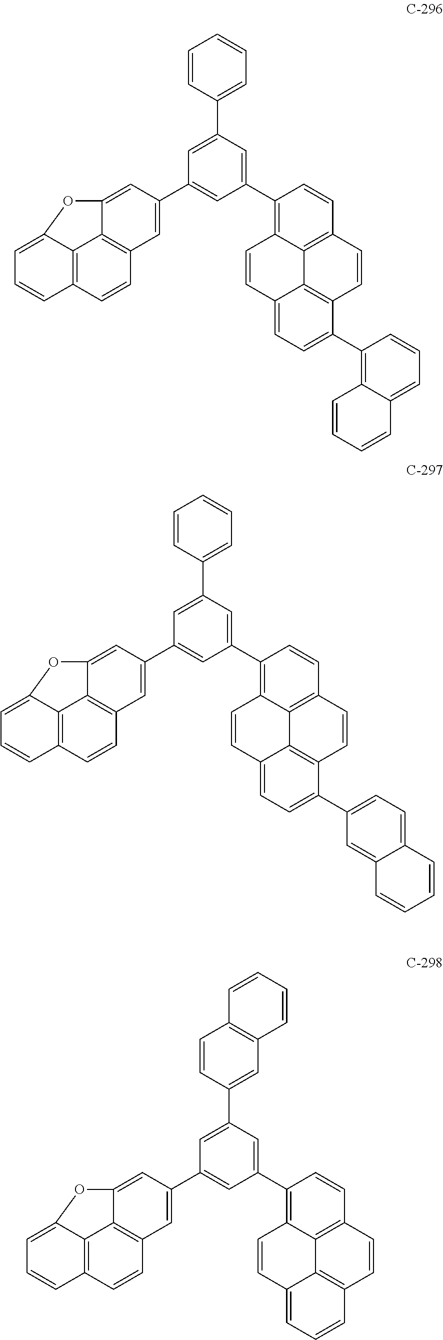 [SMILES: c1ccc(-c2cc(-c3cc4ccc5cccc6oc(c3)c4c56)cc(-c3ccc4ccc5c(-c6cccc7ccccc67)ccc6ccc3c4c65)c2)cc1 ; c1ccc(-c2cc(-c3cc4ccc5cccc6oc(c3)c4c56)cc(-c3ccc4ccc5c(-c6ccc7ccccc7c6)ccc6ccc3c4c65)c2)cc1 ; c1ccc2cc(-c3cc(-c4cc5ccc6cccc7oc(c4)c5c67)cc(-c4ccc5ccc6cccc7ccc4c5c67)c3)ccc2c1] 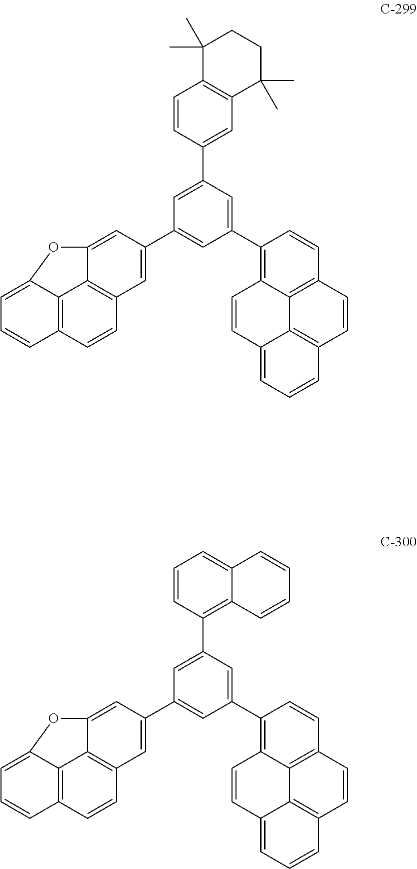 [SMILES: CC1(C)CCC(C)(C)c2cc(-c3cc(-c4cc5ccc6cccc7oc(c4)c5c67)cc(-c4ccc5ccc6cccc7ccc4c5c67)c3)ccc21 ; c1ccc2c(-c3cc(-c4cc5ccc6cccc7oc(c4)c5c67)cc(-c4ccc5ccc6cccc7ccc4c5c67)c3)cccc2c1] 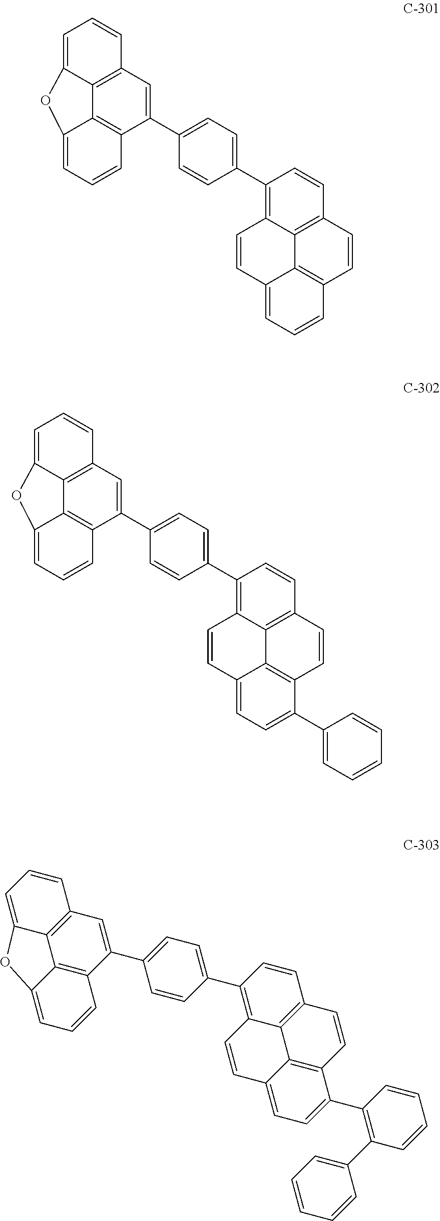 [SMILES: c1cc2ccc3ccc(-c4ccc(-c5cc6cccc7oc8cccc5c8c67)cc4)c4ccc(c1)c2c34 ; c1ccc(-c2ccc3ccc4c(-c5ccc(-c6cc7cccc8oc9cccc6c9c78)cc5)ccc5ccc2c3c54)cc1 ; c1ccc(-c2ccccc2-c2ccc3ccc4c(-c5ccc(-c6cc7cccc8oc9cccc6c9c78)cc5)ccc5ccc2c3c54)cc1] 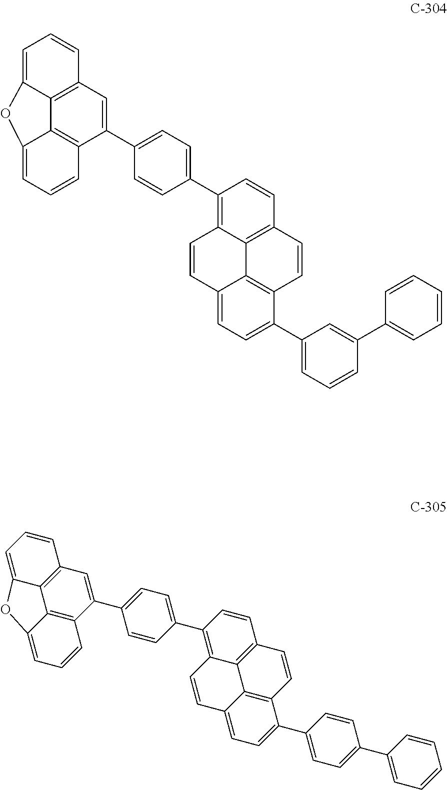 [SMILES: c1ccc(-c2cccc(-c3ccc4ccc5c(-c6ccc(-c7cc8cccc9oc%10cccc7c%10c89)cc6)ccc6ccc3c4c65)c2)cc1 ; c1ccc(-c2ccc(-c3ccc4ccc5c(-c6ccc(-c7cc8cccc9oc%10cccc7c%10c89)cc6)ccc6ccc3c4c65)cc2)cc1] 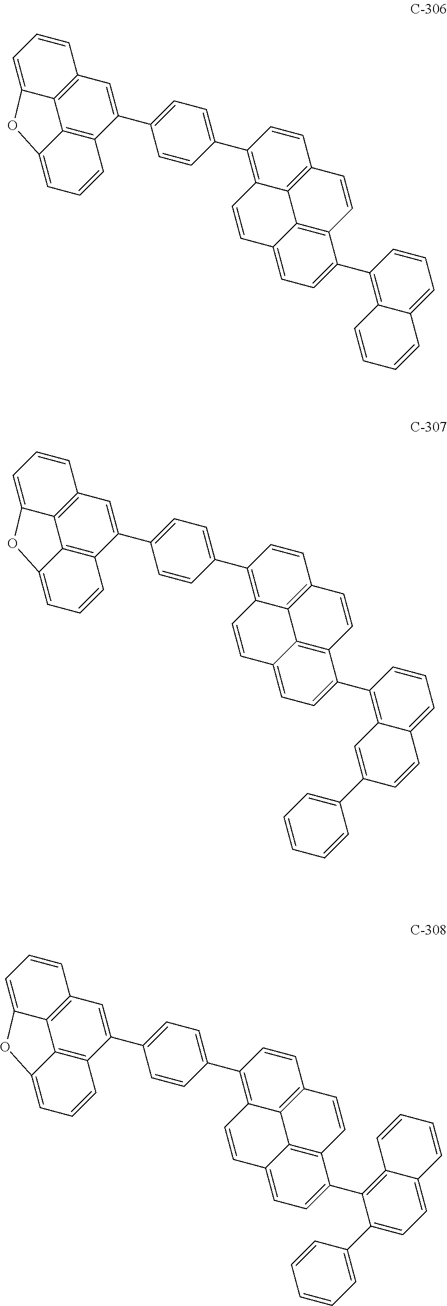 [SMILES: c1ccc2c(-c3ccc4ccc5c(-c6ccc(-c7cc8cccc9oc%10cccc7c%10c89)cc6)ccc6ccc3c4c65)cccc2c1 ; c1ccc(-c2ccc3cccc(-c4ccc5ccc6c(-c7ccc(-c8cc9cccc%10oc%11cccc8c%11c9%10)cc7)ccc7ccc4c5c76)c3c2)cc1 ; c1ccc(-c2ccc3ccccc3c2-c2ccc3ccc4c(-c5ccc(-c6cc7cccc8oc9cccc6c9c78)cc5)ccc5ccc2c3c54)cc1] 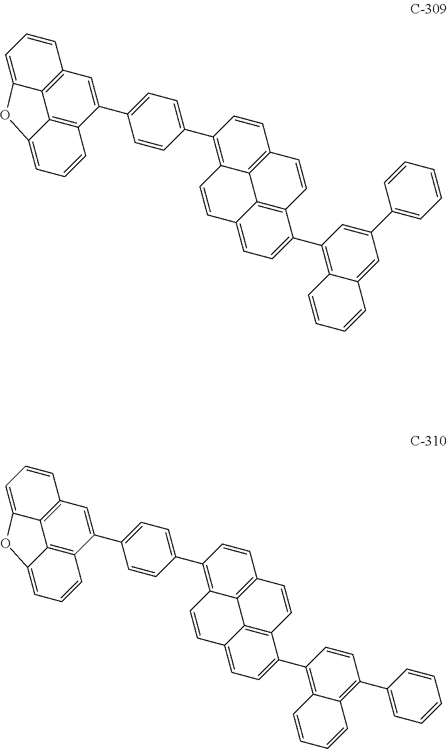 [SMILES: c1ccc(-c2cc(-c3ccc4ccc5c(-c6ccc(-c7cc8cccc9oc%10cccc7c%10c89)cc6)ccc6ccc3c4c65)c3ccccc3c2)cc1 ; c1ccc(-c2ccc(-c3ccc4ccc5c(-c6ccc(-c7cc8cccc9oc%10cccc7c%10c89)cc6)ccc6ccc3c4c65)c3ccccc23)cc1] 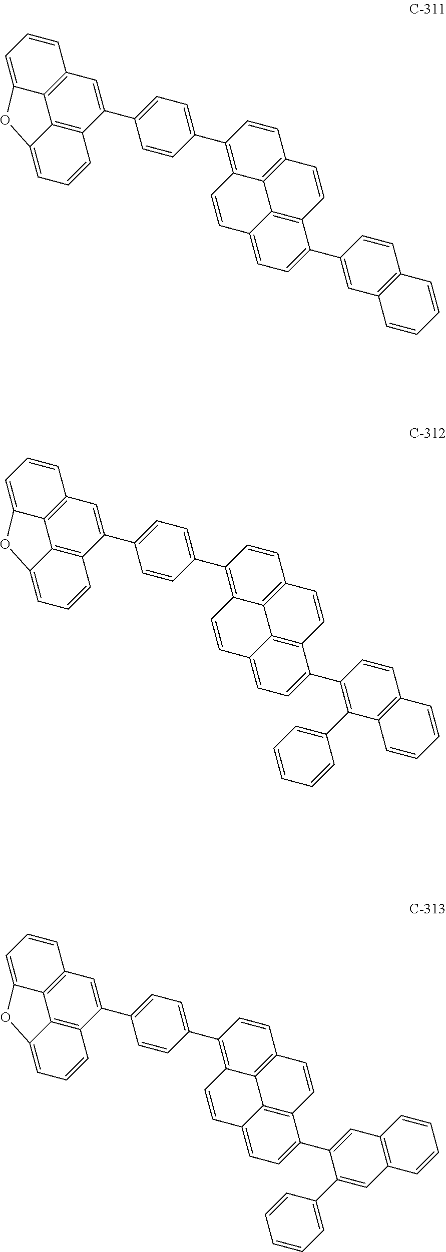 [SMILES: c1ccc2cc(-c3ccc4ccc5c(-c6ccc(-c7cc8cccc9oc%10cccc7c%10c89)cc6)ccc6ccc3c4c65)ccc2c1 ; c1ccc(-c2c(-c3ccc4ccc5c(-c6ccc(-c7cc8cccc9oc%10cccc7c%10c89)cc6)ccc6ccc3c4c65)ccc3ccccc23)cc1 ; c1ccc(-c2cc3ccccc3cc2-c2ccc3ccc4c(-c5ccc(-c6cc7cccc8oc9cccc6c9c78)cc5)ccc5ccc2c3c54)cc1] 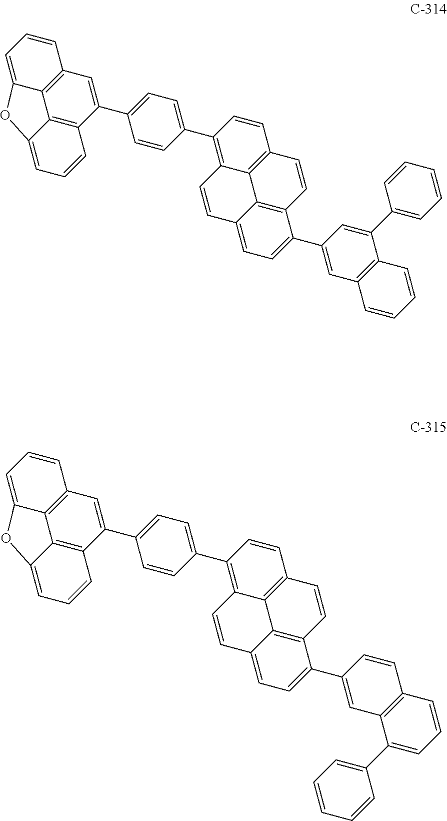 [SMILES: c1ccc(-c2cc(-c3ccc4ccc5c(-c6ccc(-c7cc8cccc9oc%10cccc7c%10c89)cc6)ccc6ccc3c4c65)cc3ccccc23)cc1 ; c1ccc(-c2cccc3ccc(-c4ccc5ccc6c(-c7ccc(-c8cc9cccc%10oc%11cccc8c%11c9%10)cc7)ccc7ccc4c5c76)cc23)cc1] 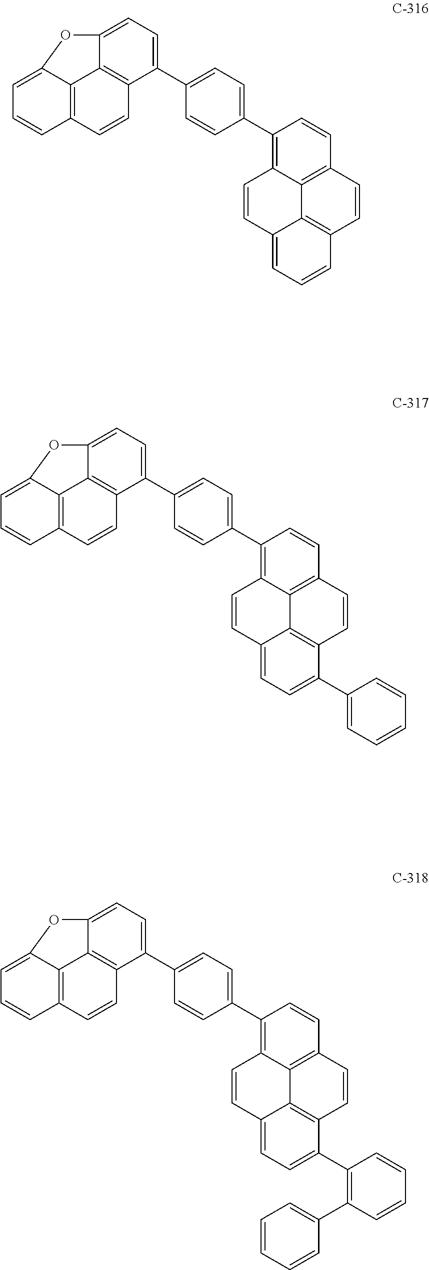 [SMILES: c1cc2ccc3ccc(-c4ccc(-c5ccc6oc7cccc8ccc5c6c87)cc4)c4ccc(c1)c2c34 ; c1ccc(-c2ccc3ccc4c(-c5ccc(-c6ccc7oc8cccc9ccc6c7c98)cc5)ccc5ccc2c3c54)cc1 ; c1ccc(-c2ccccc2-c2ccc3ccc4c(-c5ccc(-c6ccc7oc8cccc9ccc6c7c98)cc5)ccc5ccc2c3c54)cc1] 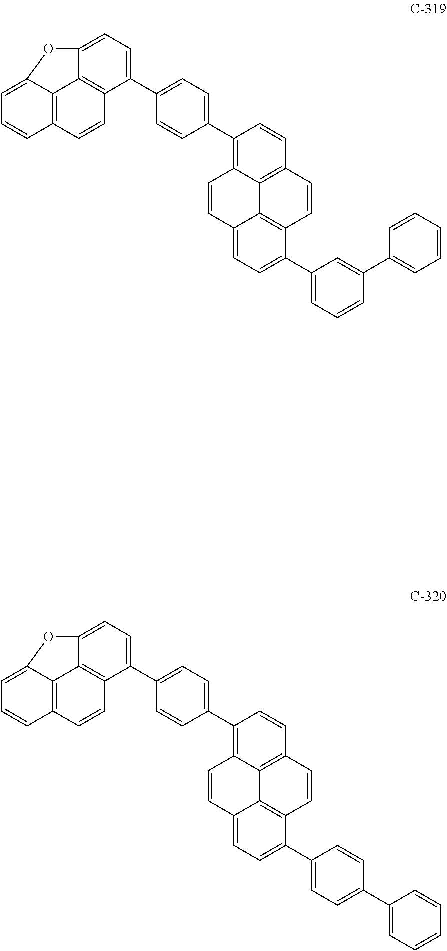 [SMILES: c1ccc(-c2cccc(-c3ccc4ccc5c(-c6ccc(-c7ccc8oc9cccc%10ccc7c8c%109)cc6)ccc6ccc3c4c65)c2)cc1 ; c1ccc(-c2ccc(-c3ccc4ccc5c(-c6ccc(-c7ccc8oc9cccc%10ccc7c8c%109)cc6)ccc6ccc3c4c65)cc2)cc1] 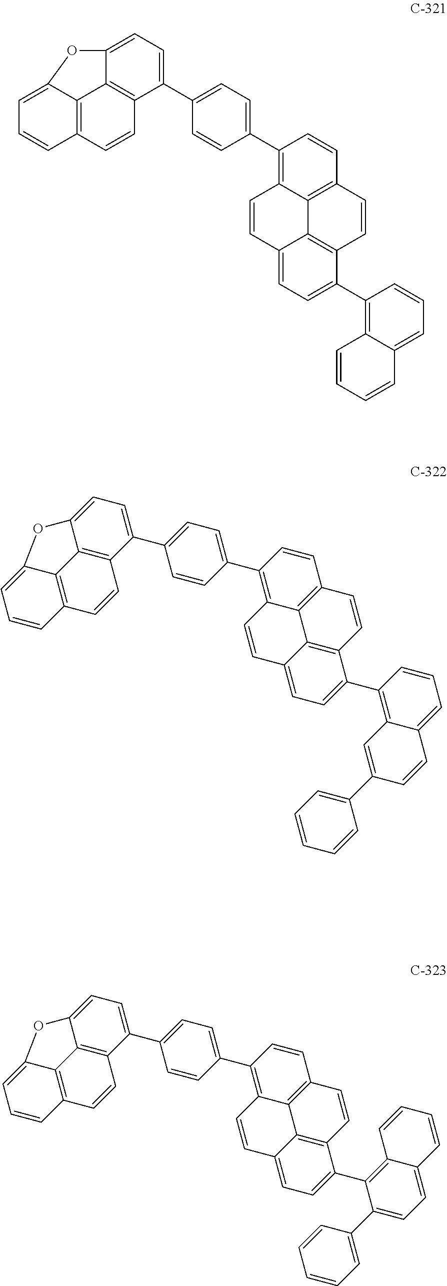 [SMILES: c1ccc2c(-c3ccc4ccc5c(-c6ccc(-c7ccc8oc9cccc%10ccc7c8c%109)cc6)ccc6ccc3c4c65)cccc2c1 ; c1ccc(-c2ccc3cccc(-c4ccc5ccc6c(-c7ccc(-c8ccc9oc%10cccc%11ccc8c9c%11%10)cc7)ccc7ccc4c5c76)c3c2)cc1 ; c1ccc(-c2ccc3ccccc3c2-c2ccc3ccc4c(-c5ccc(-c6ccc7oc8cccc9ccc6c7c98)cc5)ccc5ccc2c3c54)cc1] 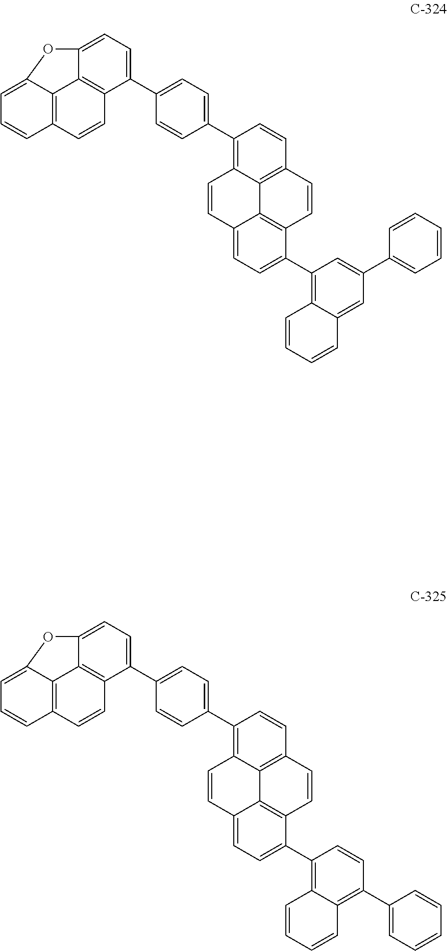 [SMILES: c1ccc(-c2cc(-c3ccc4ccc5c(-c6ccc(-c7ccc8oc9cccc%10ccc7c8c%109)cc6)ccc6ccc3c4c65)c3ccccc3c2)cc1 ; c1ccc(-c2ccc(-c3ccc4ccc5c(-c6ccc(-c7ccc8oc9cccc%10ccc7c8c%109)cc6)ccc6ccc3c4c65)c3ccccc23)cc1] 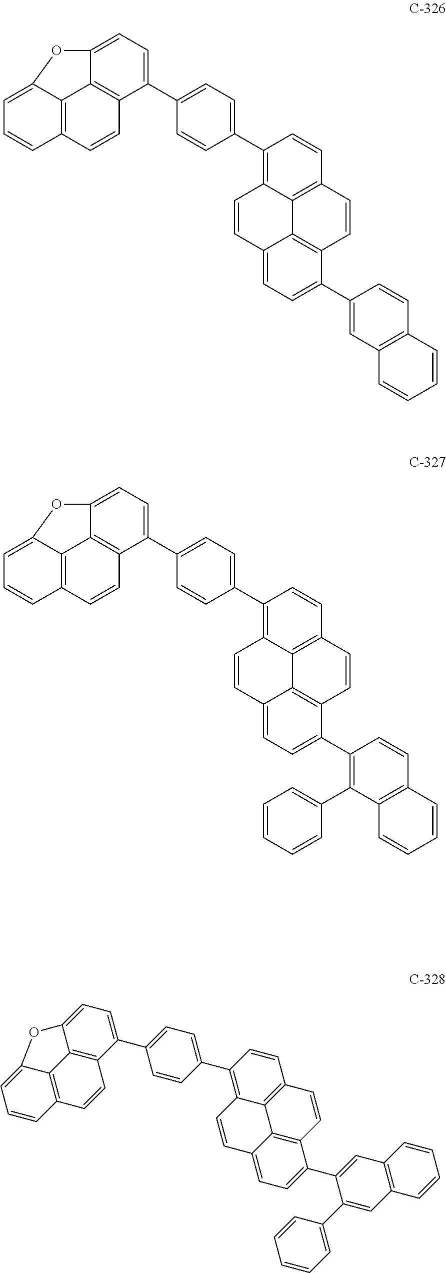 [SMILES: c1ccc2cc(-c3ccc4ccc5c(-c6ccc(-c7ccc8oc9cccc%10ccc7c8c%109)cc6)ccc6ccc3c4c65)ccc2c1 ; c1ccc(-c2c(-c3ccc4ccc5c(-c6ccc(-c7ccc8oc9cccc%10ccc7c8c%109)cc6)ccc6ccc3c4c65)ccc3ccccc23)cc1 ; c1ccc(-c2cc3ccccc3cc2-c2ccc3ccc4c(-c5ccc(-c6ccc7oc8cccc9ccc6c7c98)cc5)ccc5ccc2c3c54)cc1] 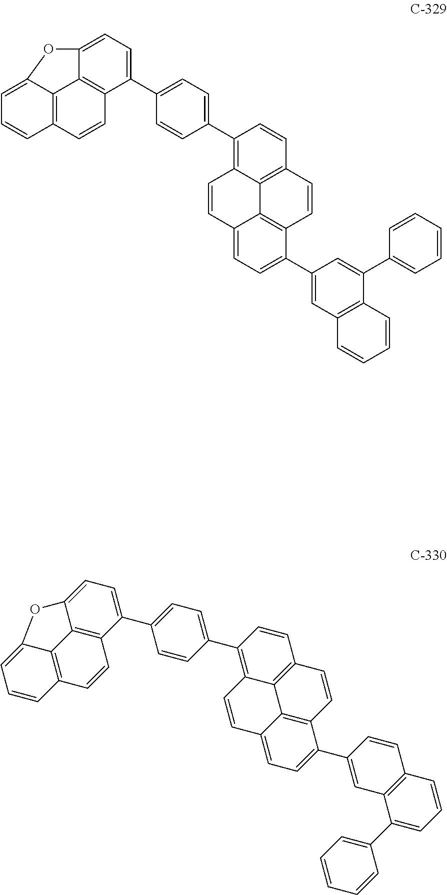 [SMILES: c1ccc(-c2cc(-c3ccc4ccc5c(-c6ccc(-c7ccc8oc9cccc%10ccc7c8c%109)cc6)ccc6ccc3c4c65)cc3ccccc23)cc1 ; c1ccc(-c2cccc3ccc(-c4ccc5ccc6c(-c7ccc(-c8ccc9oc%10cccc%11ccc8c9c%11%10)cc7)ccc7ccc4c5c76)cc23)cc1] 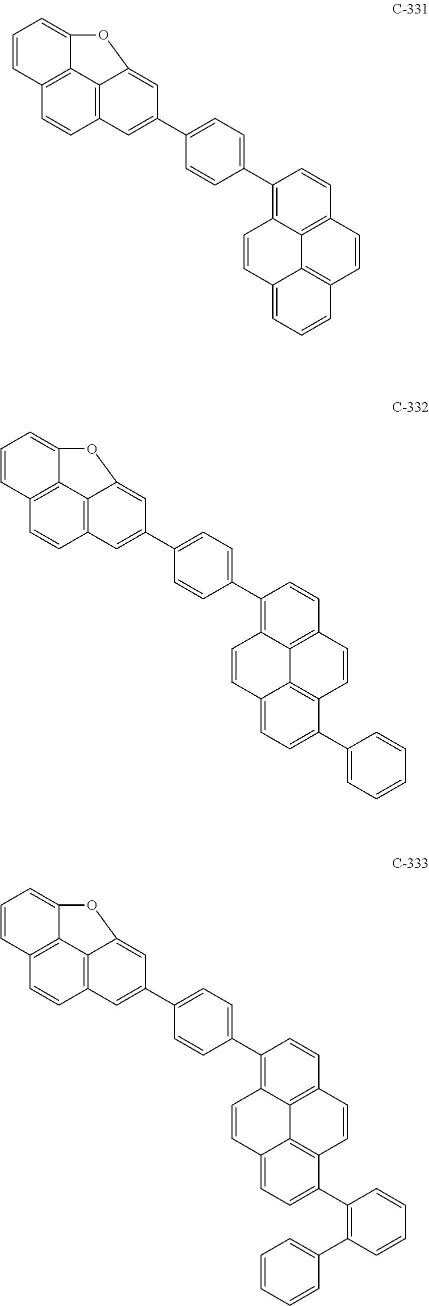 [SMILES: c1cc2ccc3ccc(-c4ccc(-c5cc6ccc7cccc8oc(c5)c6c78)cc4)c4ccc(c1)c2c34 ; c1ccc(-c2ccc3ccc4c(-c5ccc(-c6cc7ccc8cccc9oc(c6)c7c89)cc5)ccc5ccc2c3c54)cc1 ; c1ccc(-c2ccccc2-c2ccc3ccc4c(-c5ccc(-c6cc7ccc8cccc9oc(c6)c7c89)cc5)ccc5ccc2c3c54)cc1] 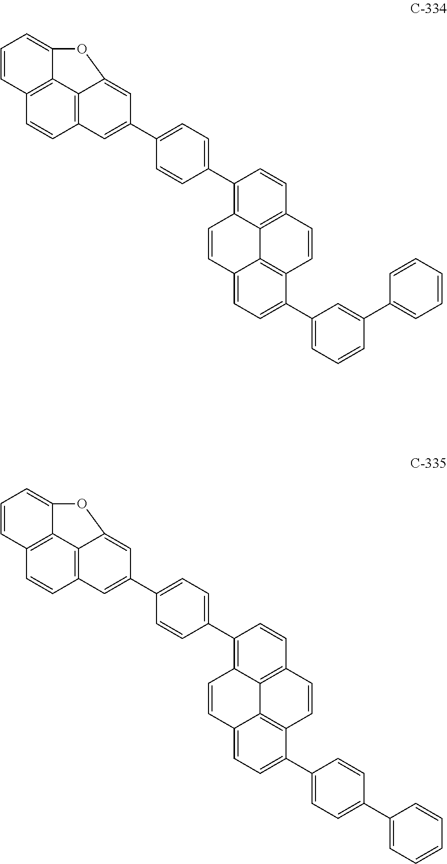 [SMILES: c1ccc(-c2cccc(-c3ccc4ccc5c(-c6ccc(-c7cc8ccc9cccc%10oc(c7)c8c9%10)cc6)ccc6ccc3c4c65)c2)cc1 ; c1ccc(-c2ccc(-c3ccc4ccc5c(-c6ccc(-c7cc8ccc9cccc%10oc(c7)c8c9%10)cc6)ccc6ccc3c4c65)cc2)cc1] 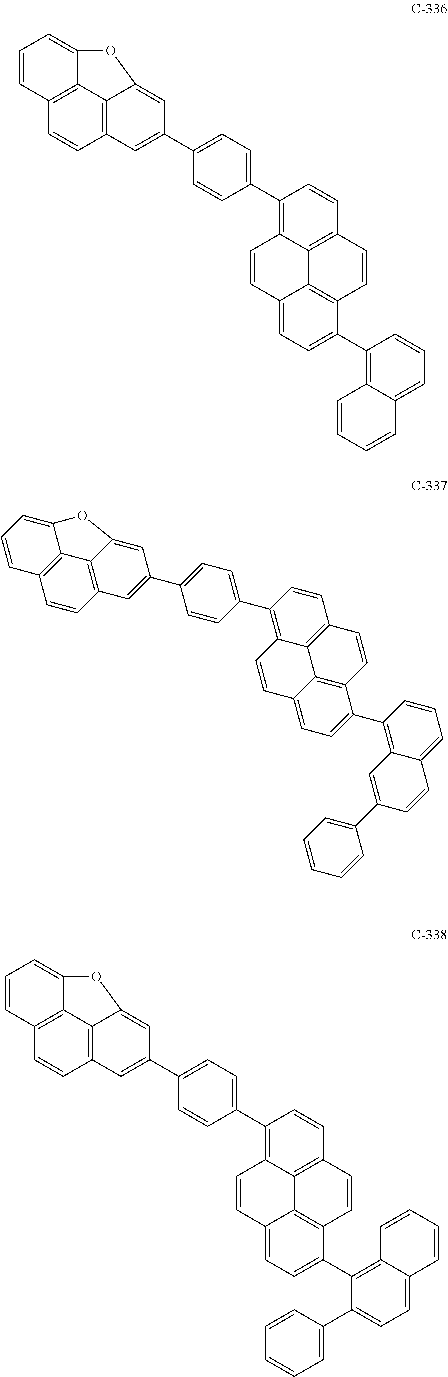 [SMILES: c1ccc2c(-c3ccc4ccc5c(-c6ccc(-c7cc8ccc9cccc%10oc(c7)c8c9%10)cc6)ccc6ccc3c4c65)cccc2c1 ; c1ccc(-c2ccc3cccc(-c4ccc5ccc6c(-c7ccc(-c8cc9ccc%10cccc%11oc(c8)c9c%10%11)cc7)ccc7ccc4c5c76)c3c2)cc1 ; c1ccc(-c2ccc3ccccc3c2-c2ccc3ccc4c(-c5ccc(-c6cc7ccc8cccc9oc(c6)c7c89)cc5)ccc5ccc2c3c54)cc1] 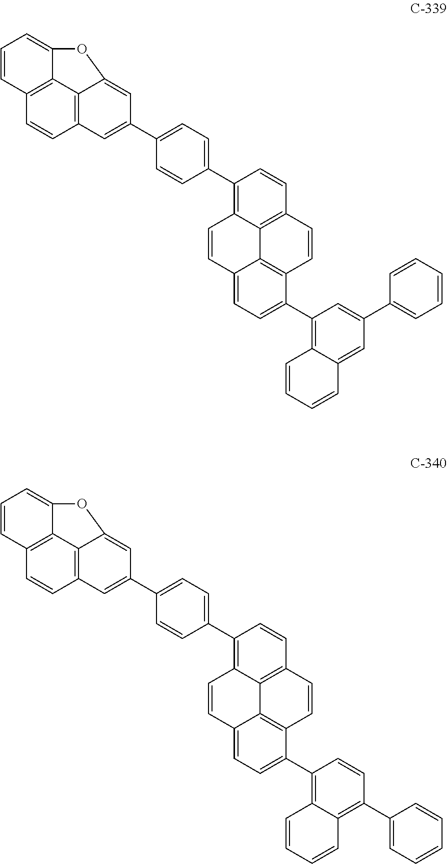 [SMILES: c1ccc(-c2cc(-c3ccc4ccc5c(-c6ccc(-c7cc8ccc9cccc%10oc(c7)c8c9%10)cc6)ccc6ccc3c4c65)c3ccccc3c2)cc1 ; c1ccc(-c2ccc(-c3ccc4ccc5c(-c6ccc(-c7cc8ccc9cccc%10oc(c7)c8c9%10)cc6)ccc6ccc3c4c65)c3ccccc23)cc1] 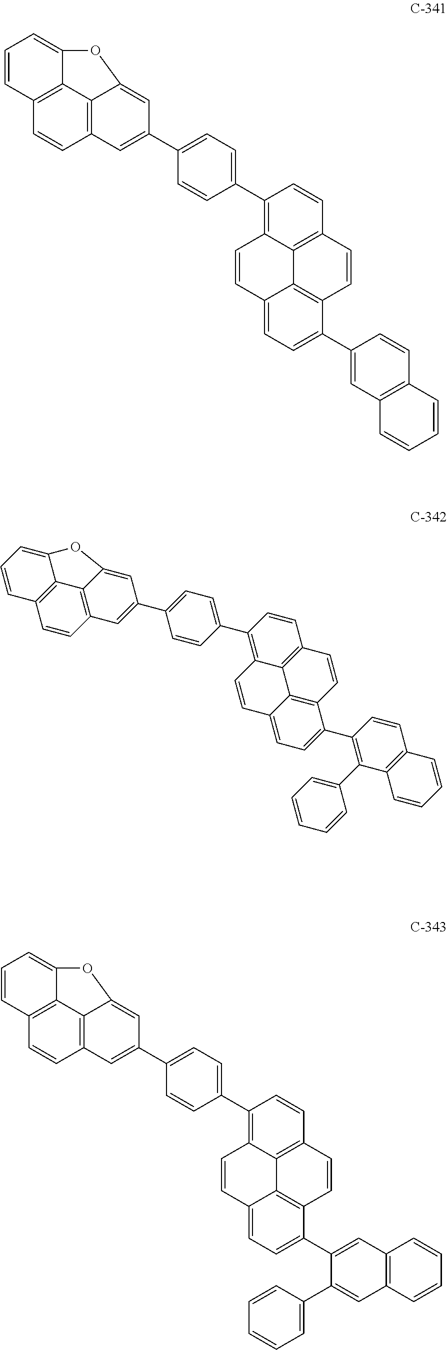 [SMILES: c1ccc2cc(-c3ccc4ccc5c(-c6ccc(-c7cc8ccc9cccc%10oc(c7)c8c9%10)cc6)ccc6ccc3c4c65)ccc2c1 ; c1ccc(-c2c(-c3ccc4ccc5c(-c6ccc(-c7cc8ccc9cccc%10oc(c7)c8c9%10)cc6)ccc6ccc3c4c65)ccc3ccccc23)cc1 ; c1ccc(-c2cc3ccccc3cc2-c2ccc3ccc4c(-c5ccc(-c6cc7ccc8cccc9oc(c6)c7c89)cc5)ccc5ccc2c3c54)cc1] 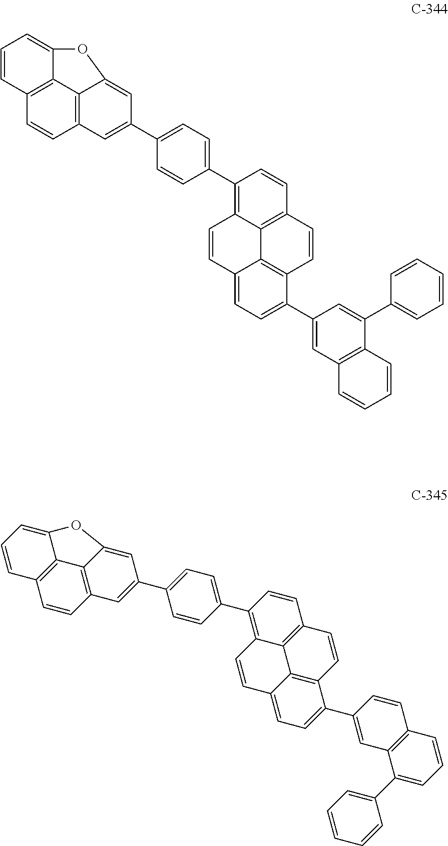 [SMILES: c1ccc(-c2cc(-c3ccc4ccc5c(-c6ccc(-c7cc8ccc9cccc%10oc(c7)c8c9%10)cc6)ccc6ccc3c4c65)cc3ccccc23)cc1 ; c1ccc(-c2cccc3ccc(-c4ccc5ccc6c(-c7ccc(-c8cc9ccc%10cccc%11oc(c8)c9c%10%11)cc7)ccc7ccc4c5c76)cc23)cc1] 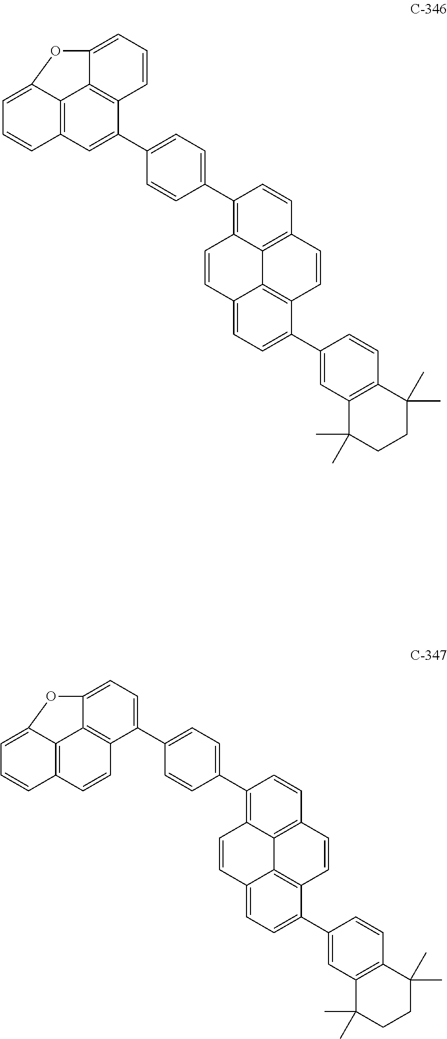 [SMILES: CC1(C)CCC(C)(C)c2cc(-c3ccc4ccc5c(-c6ccc(-c7cc8cccc9oc%10cccc7c%10c89)cc6)ccc6ccc3c4c65)ccc21 ; CC1(C)CCC(C)(C)c2cc(-c3ccc4ccc5c(-c6ccc(-c7ccc8oc9cccc%10ccc7c8c%109)cc6)ccc6ccc3c4c65)ccc21] 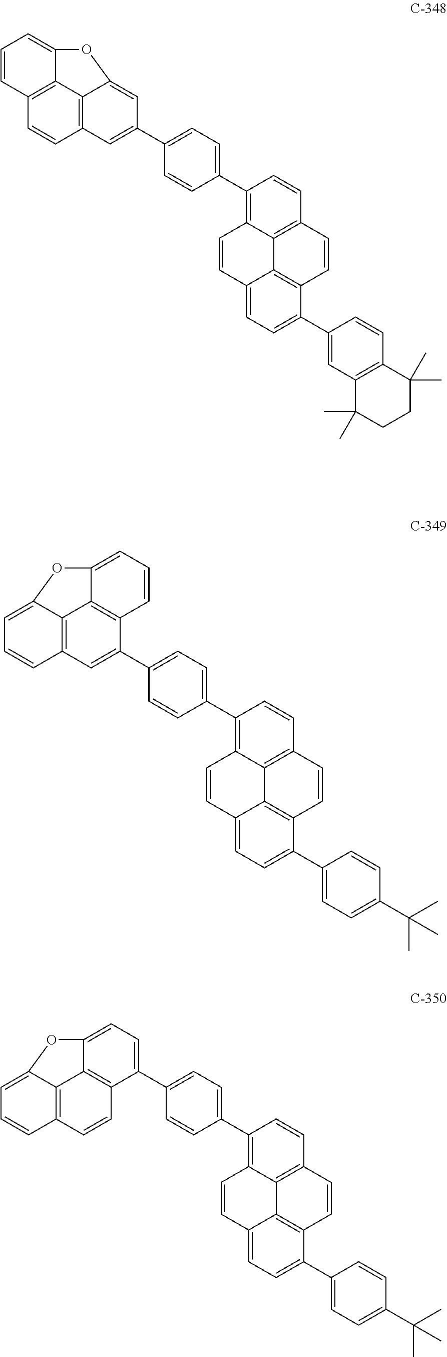 [SMILES: CC1(C)CCC(C)(C)c2cc(-c3ccc4ccc5c(-c6ccc(-c7cc8ccc9cccc%10oc(c7)c8c9%10)cc6)ccc6ccc3c4c65)ccc21 ; CC(C)(C)c1ccc(-c2ccc3ccc4c(-c5ccc(-c6cc7cccc8oc9cccc6c9c78)cc5)ccc5ccc2c3c54)cc1 ; CC(C)(C)c1ccc(-c2ccc3ccc4c(-c5ccc(-c6ccc7oc8cccc9ccc6c7c98)cc5)ccc5ccc2c3c54)cc1] 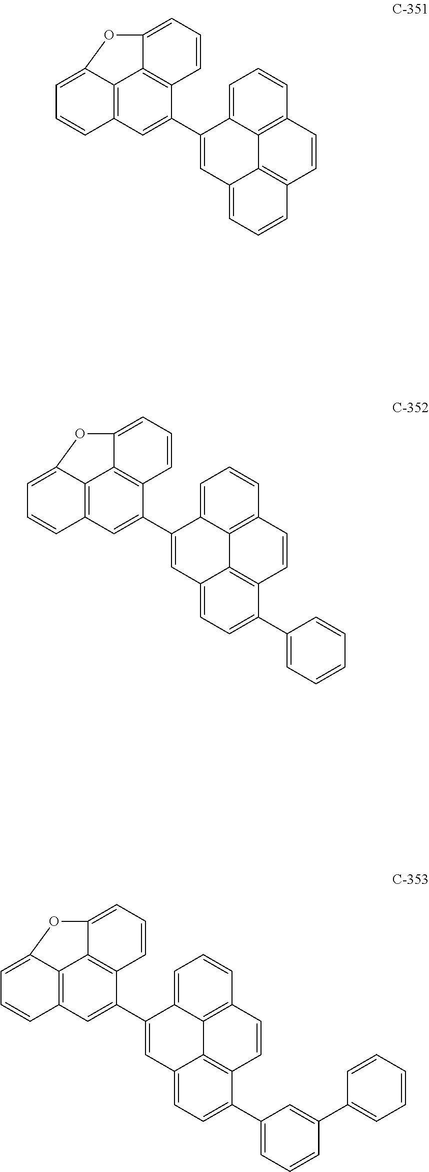 [SMILES: c1cc2ccc3cccc4c(-c5cc6cccc7oc8cccc5c8c67)cc(c1)c2c34 ; c1ccc(-c2ccc3cc(-c4cc5cccc6oc7cccc4c7c56)c4cccc5ccc2c3c54)cc1 ; c1ccc(-c2cccc(-c3ccc4cc(-c5cc6cccc7oc8cccc5c8c67)c5cccc6ccc3c4c65)c2)cc1] 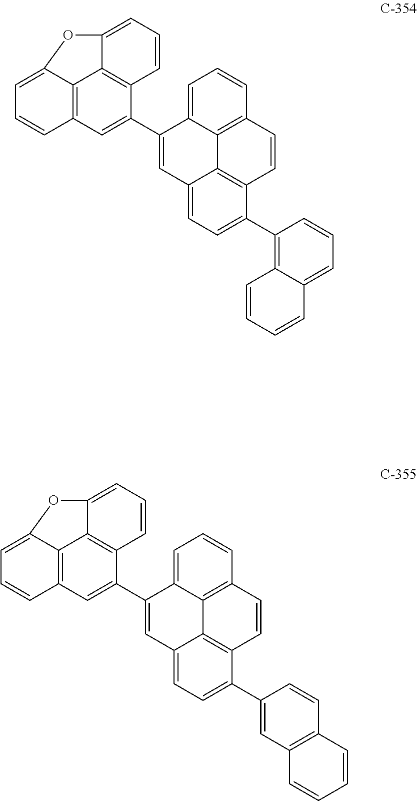 [SMILES: c1ccc2c(-c3ccc4cc(-c5cc6cccc7oc8cccc5c8c67)c5cccc6ccc3c4c65)cccc2c1 ; c1ccc2cc(-c3ccc4cc(-c5cc6cccc7oc8cccc5c8c67)c5cccc6ccc3c4c65)ccc2c1] 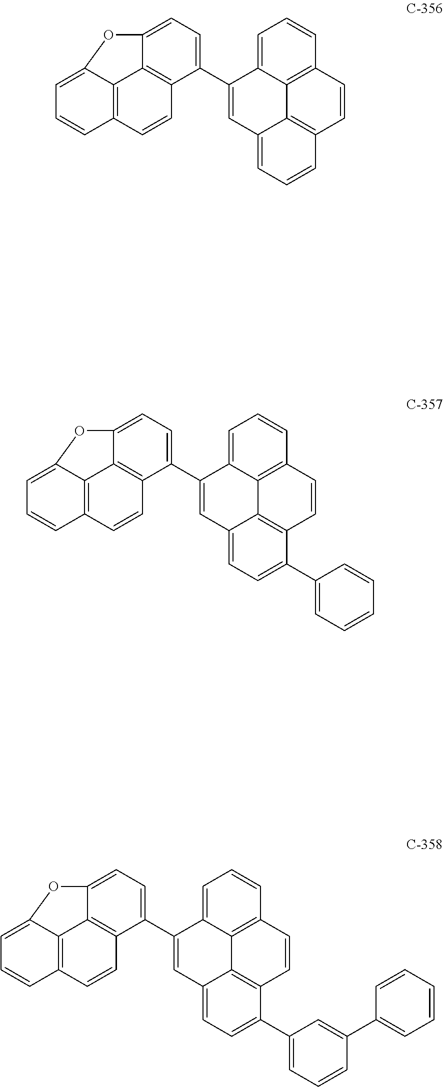 [SMILES: c1cc2ccc3cccc4c(-c5ccc6oc7cccc8ccc5c6c87)cc(c1)c2c34 ; c1ccc(-c2ccc3cc(-c4ccc5oc6cccc7ccc4c5c76)c4cccc5ccc2c3c54)cc1 ; c1ccc(-c2cccc(-c3ccc4cc(-c5ccc6oc7cccc8ccc5c6c87)c5cccc6ccc3c4c65)c2)cc1] 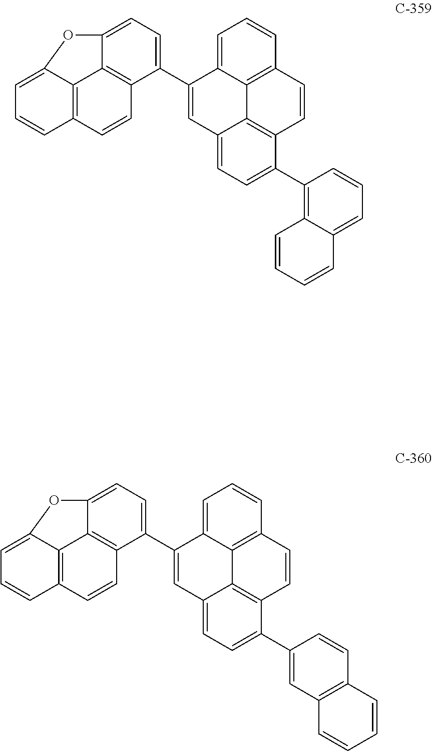 [SMILES: c1ccc2c(-c3ccc4cc(-c5ccc6oc7cccc8ccc5c6c87)c5cccc6ccc3c4c65)cccc2c1 ; c1ccc2cc(-c3ccc4cc(-c5ccc6oc7cccc8ccc5c6c87)c5cccc6ccc3c4c65)ccc2c1] 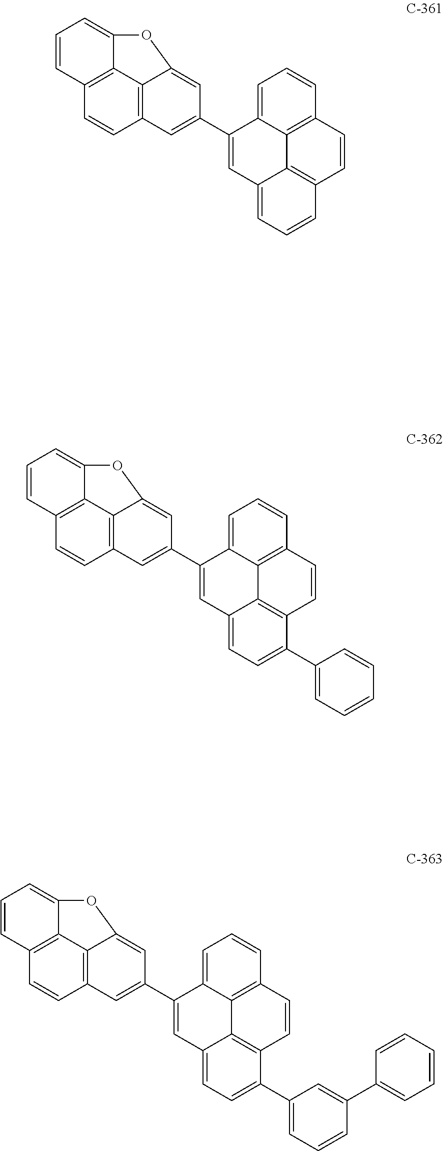 [SMILES: c1cc2ccc3cccc4c(-c5cc6ccc7cccc8oc(c5)c6c78)cc(c1)c2c34 ; c1ccc(-c2ccc3cc(-c4cc5ccc6cccc7oc(c4)c5c67)c4cccc5ccc2c3c54)cc1 ; c1ccc(-c2cccc(-c3ccc4cc(-c5cc6ccc7cccc8oc(c5)c6c78)c5cccc6ccc3c4c65)c2)cc1] 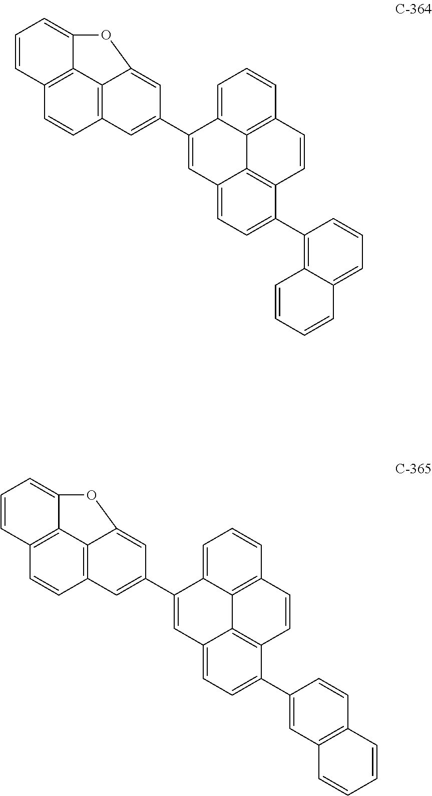 [SMILES: c1ccc2c(-c3ccc4cc(-c5cc6ccc7cccc8oc(c5)c6c78)c5cccc6ccc3c4c65)cccc2c1 ; c1ccc2cc(-c3ccc4cc(-c5cc6ccc7cccc8oc(c5)c6c78)c5cccc6ccc3c4c65)ccc2c1] 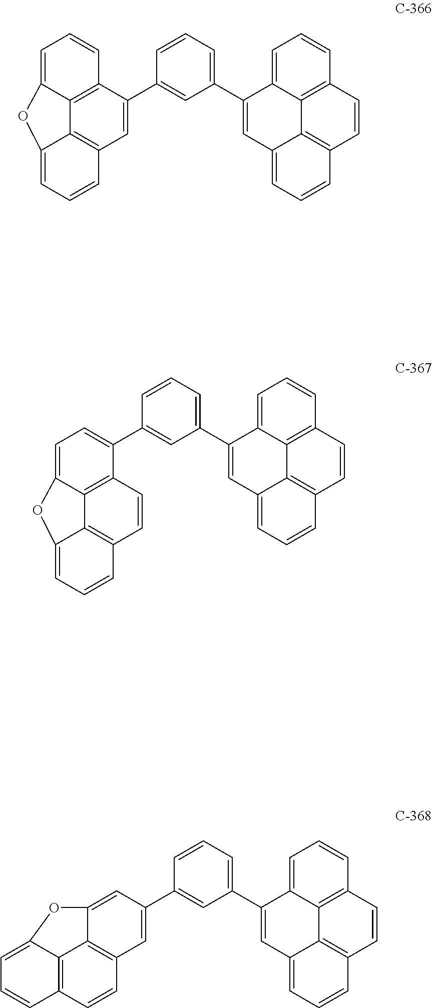 [SMILES: c1cc(-c2cc3cccc4ccc5cccc2c5c43)cc(-c2cc3cccc4oc5cccc2c5c34)c1 ; c1cc(-c2cc3cccc4ccc5cccc2c5c43)cc(-c2ccc3oc4cccc5ccc2c3c54)c1 ; c1cc(-c2cc3ccc4cccc5oc(c2)c3c45)cc(-c2cc3cccc4ccc5cccc2c5c43)c1] 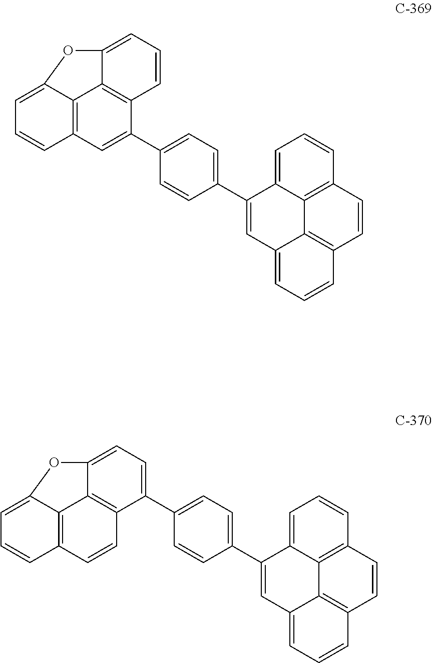 [SMILES: c1cc2ccc3cccc4c(-c5ccc(-c6cc7cccc8oc9cccc6c9c78)cc5)cc(c1)c2c34 ; c1cc2ccc3cccc4c(-c5ccc(-c6ccc7oc8cccc9ccc6c7c98)cc5)cc(c1)c2c34] 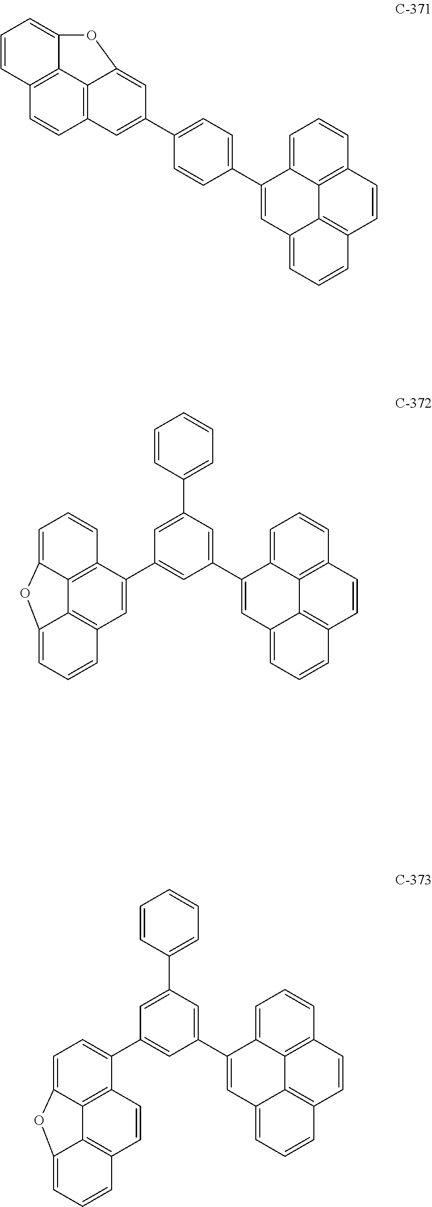 [SMILES: c1cc2ccc3cccc4c(-c5ccc(-c6cc7ccc8cccc9oc(c6)c7c89)cc5)cc(c1)c2c34 ; c1ccc(-c2cc(-c3cc4cccc5ccc6cccc3c6c54)cc(-c3cc4cccc5oc6cccc3c6c45)c2)cc1 ; c1ccc(-c2cc(-c3cc4cccc5ccc6cccc3c6c54)cc(-c3ccc4oc5cccc6ccc3c4c65)c2)cc1] 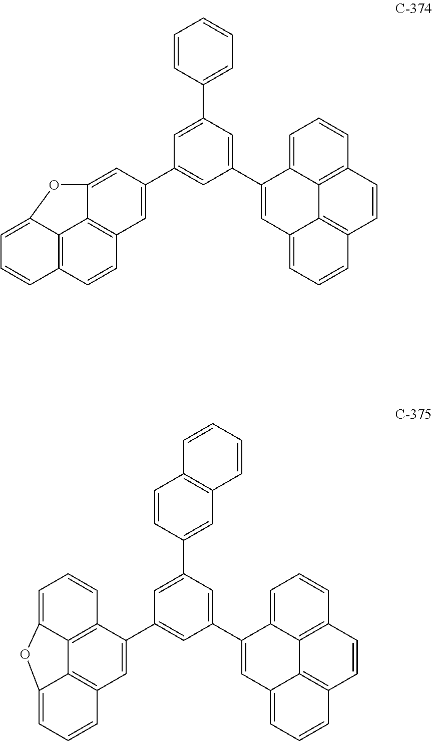 [SMILES: c1ccc(-c2cc(-c3cc4ccc5cccc6oc(c3)c4c56)cc(-c3cc4cccc5ccc6cccc3c6c54)c2)cc1 ; c1ccc2cc(-c3cc(-c4cc5cccc6ccc7cccc4c7c65)cc(-c4cc5cccc6oc7cccc4c7c56)c3)ccc2c1] 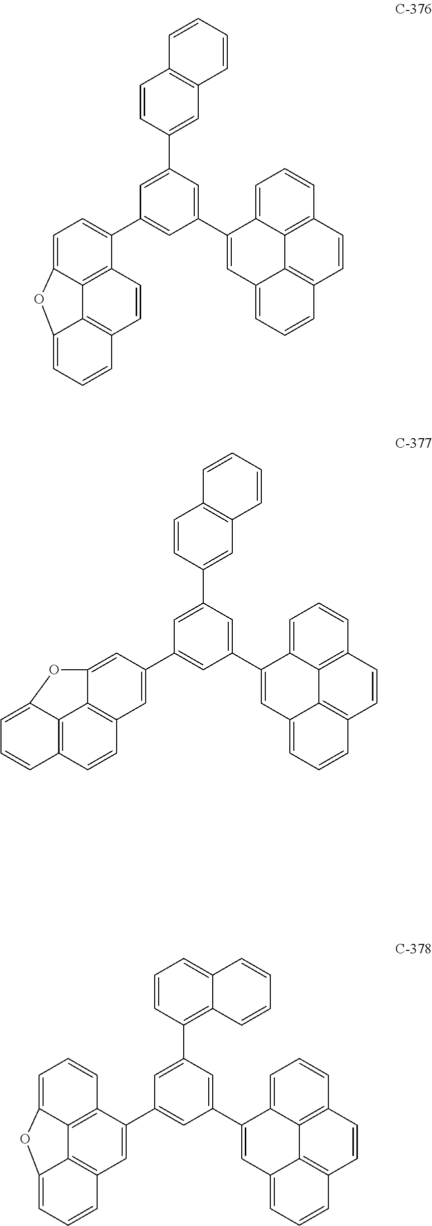 [SMILES: c1ccc2cc(-c3cc(-c4cc5cccc6ccc7cccc4c7c65)cc(-c4ccc5oc6cccc7ccc4c5c76)c3)ccc2c1 ; c1ccc2cc(-c3cc(-c4cc5ccc6cccc7oc(c4)c5c67)cc(-c4cc5cccc6ccc7cccc4c7c65)c3)ccc2c1 ; c1ccc2c(-c3cc(-c4cc5cccc6ccc7cccc4c7c65)cc(-c4cc5cccc6oc7cccc4c7c56)c3)cccc2c1] 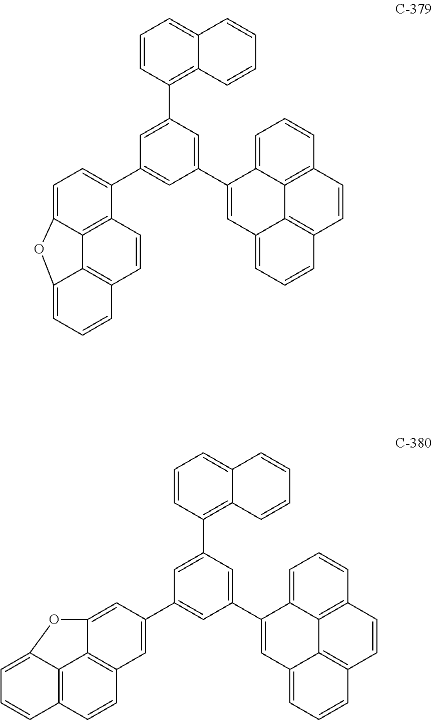 [SMILES: c1ccc2c(-c3cc(-c4cc5cccc6ccc7cccc4c7c65)cc(-c4ccc5oc6cccc7ccc4c5c76)c3)cccc2c1 ; c1ccc2c(-c3cc(-c4cc5ccc6cccc7oc(c4)c5c67)cc(-c4cc5cccc6ccc7cccc4c7c65)c3)cccc2c1] 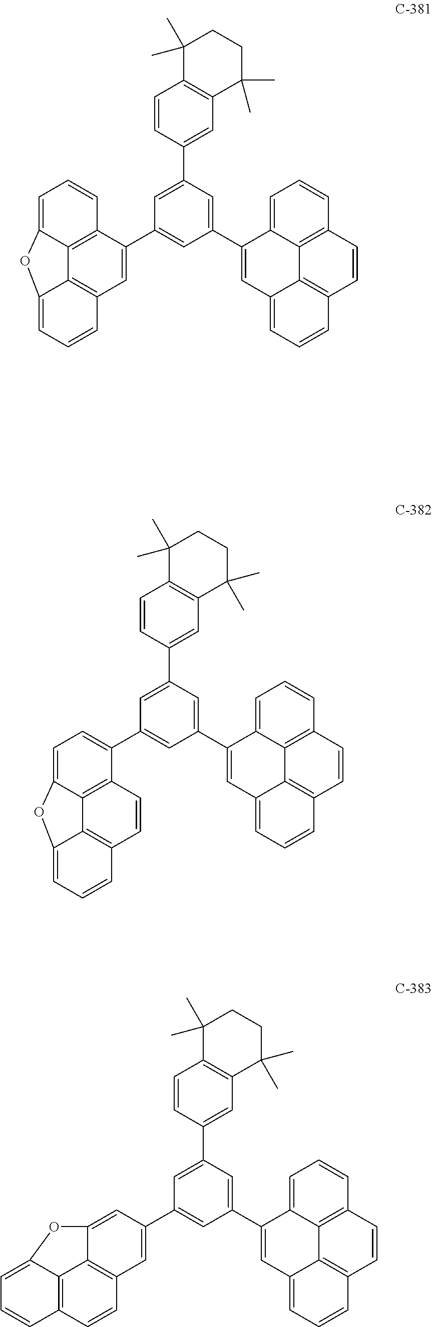 [SMILES: CC1(C)CCC(C)(C)c2cc(-c3cc(-c4cc5cccc6ccc7cccc4c7c65)cc(-c4cc5cccc6oc7cccc4c7c56)c3)ccc21 ; CC1(C)CCC(C)(C)c2cc(-c3cc(-c4cc5cccc6ccc7cccc4c7c65)cc(-c4ccc5oc6cccc7ccc4c5c76)c3)ccc21 ; CC1(C)CCC(C)(C)c2cc(-c3cc(-c4cc5ccc6cccc7oc(c4)c5c67)cc(-c4cc5cccc6ccc7cccc4c7c65)c3)ccc21] 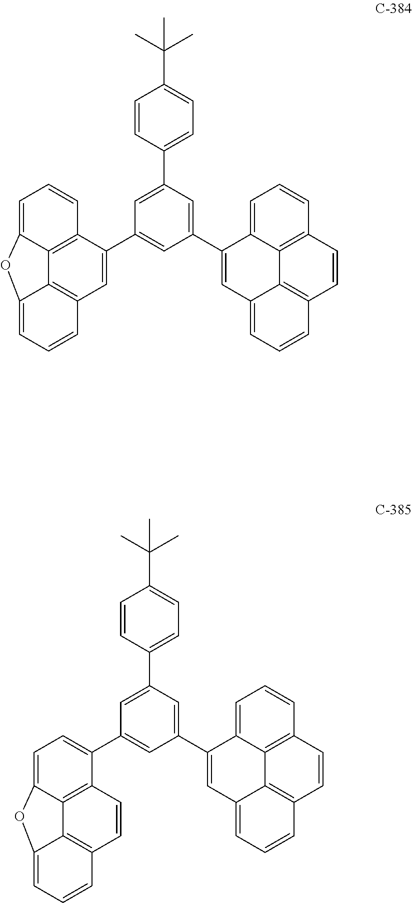 [SMILES: CC(C)(C)c1ccc(-c2cc(-c3cc4cccc5ccc6cccc3c6c54)cc(-c3cc4cccc5oc6cccc3c6c45)c2)cc1 ; CC(C)(C)c1ccc(-c2cc(-c3cc4cccc5ccc6cccc3c6c54)cc(-c3ccc4oc5cccc6ccc3c4c65)c2)cc1] 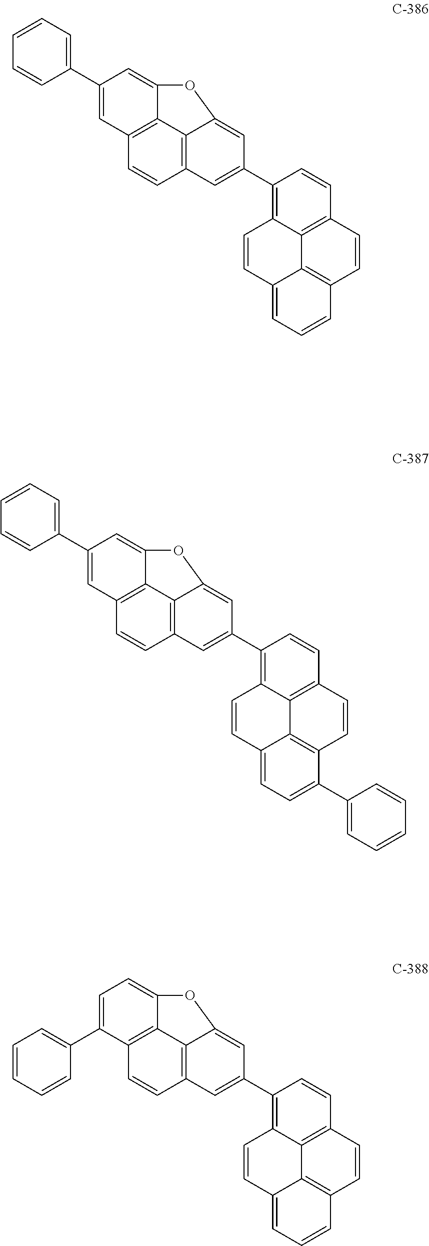 [SMILES: c1ccc(-c2cc3ccc4cc(-c5ccc6ccc7cccc8ccc5c6c78)cc5oc(c2)c3c45)cc1 ; c1ccc(-c2cc3ccc4cc(-c5ccc6ccc7c(-c8ccccc8)ccc8ccc5c6c87)cc5oc(c2)c3c45)cc1 ; c1ccc(-c2ccc3oc4cc(-c5ccc6ccc7cccc8ccc5c6c78)cc5ccc2c3c54)cc1] 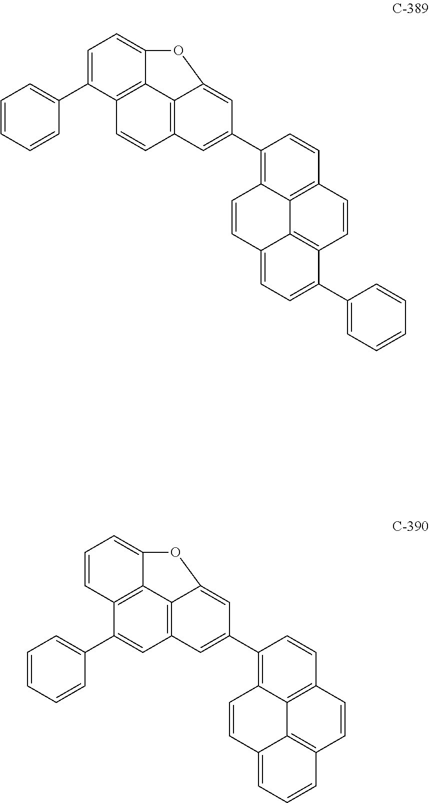 [SMILES: c1ccc(-c2ccc3ccc4c(-c5cc6ccc7c(-c8ccccc8)ccc8oc(c5)c6c87)ccc5ccc2c3c54)cc1 ; c1ccc(-c2cc3cc(-c4ccc5ccc6cccc7ccc4c5c67)cc4oc5cccc2c5c34)cc1] 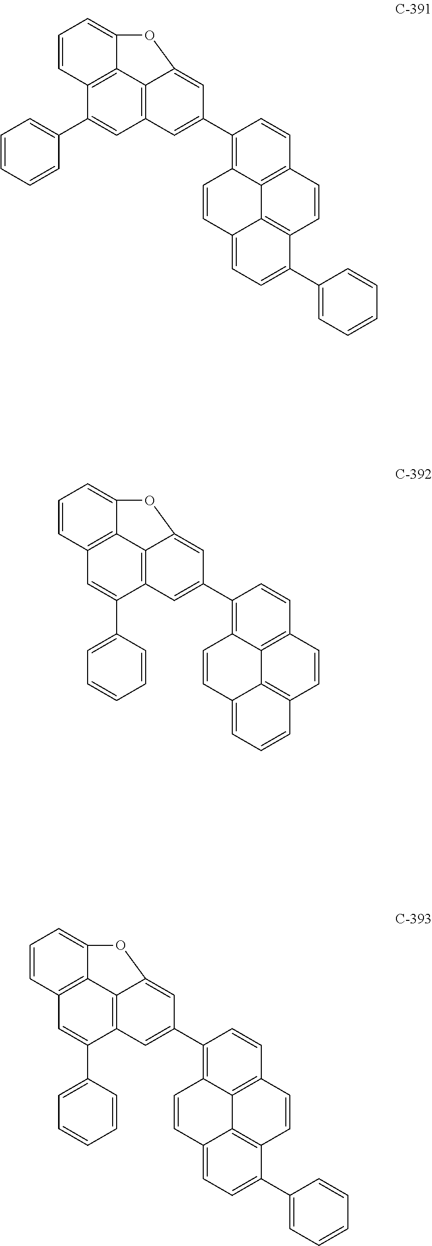 [SMILES: c1ccc(-c2ccc3ccc4c(-c5cc6cc(-c7ccccc7)c7cccc8oc(c5)c6c87)ccc5ccc2c3c54)cc1 ; c1ccc(-c2cc3cccc4oc5cc(-c6ccc7ccc8cccc9ccc6c7c89)cc2c5c34)cc1 ; c1ccc(-c2ccc3ccc4c(-c5cc6oc7cccc8cc(-c9ccccc9)c(c5)c6c87)ccc5ccc2c3c54)cc1] 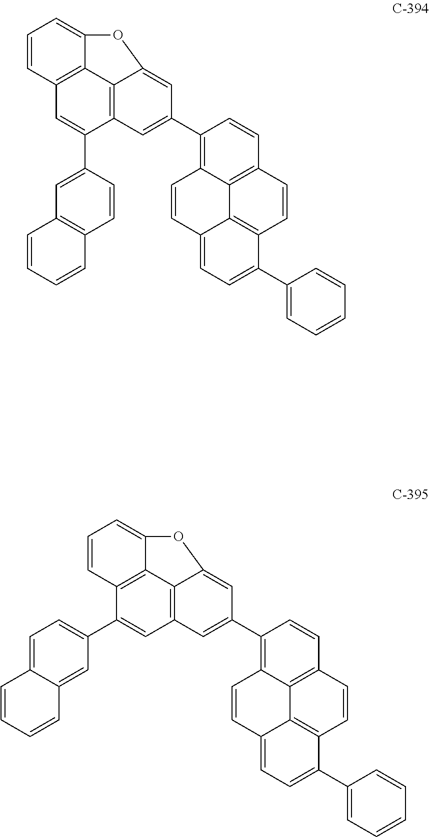 [SMILES: c1ccc(-c2ccc3ccc4c(-c5cc6oc7cccc8cc(-c9ccc%10ccccc%10c9)c(c5)c6c87)ccc5ccc2c3c54)cc1 ; c1ccc(-c2ccc3ccc4c(-c5cc6cc(-c7ccc8ccccc8c7)c7cccc8oc(c5)c6c87)ccc5ccc2c3c54)cc1] 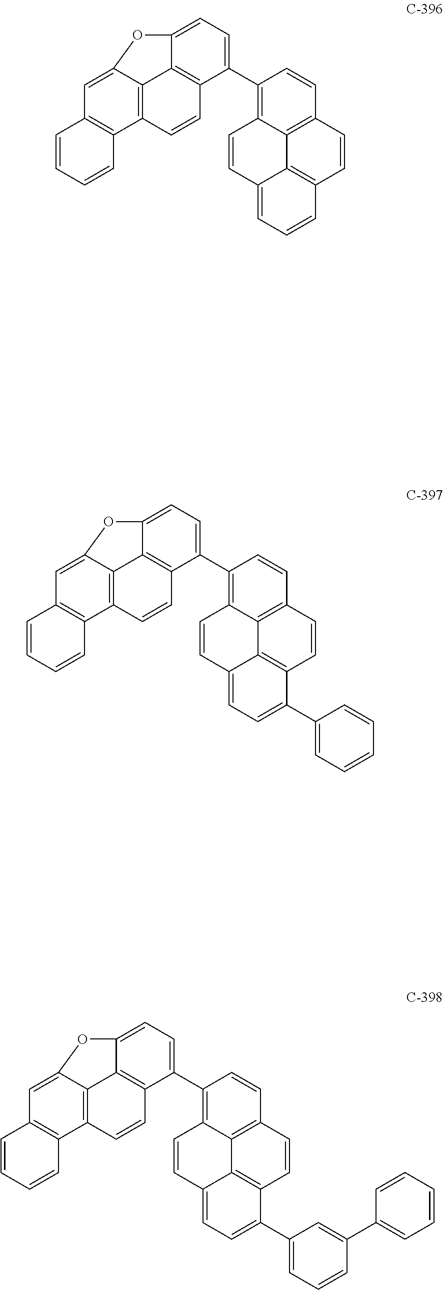 [SMILES: c1ccc2c(c1)cc1oc3ccc(-c4ccc5ccc6cccc7ccc4c5c67)c4ccc2c1c34 ; c1ccc(-c2ccc3ccc4c(-c5ccc6oc7cc8ccccc8c8ccc5c6c78)ccc5ccc2c3c54)cc1 ; c1ccc(-c2cccc(-c3ccc4ccc5c(-c6ccc7oc8cc9ccccc9c9ccc6c7c89)ccc6ccc3c4c65)c2)cc1] 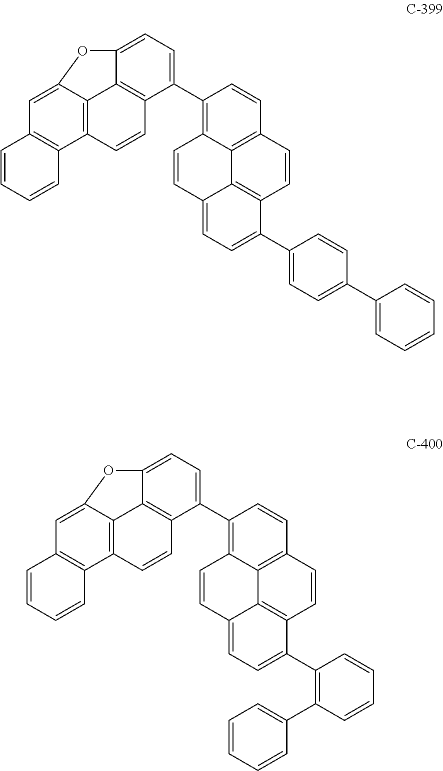 [SMILES: c1ccc(-c2ccc(-c3ccc4ccc5c(-c6ccc7oc8cc9ccccc9c9ccc6c7c89)ccc6ccc3c4c65)cc2)cc1 ; c1ccc(-c2ccccc2-c2ccc3ccc4c(-c5ccc6oc7cc8ccccc8c8ccc5c6c78)ccc5ccc2c3c54)cc1] 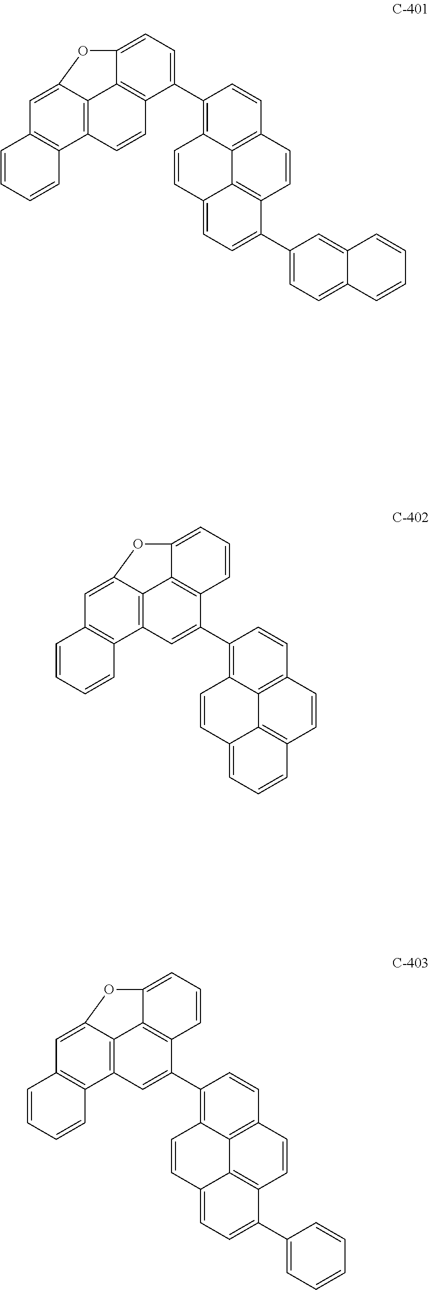 [SMILES: c1ccc2cc(-c3ccc4ccc5c(-c6ccc7oc8cc9ccccc9c9ccc6c7c89)ccc6ccc3c4c65)ccc2c1 ; c1ccc2c(c1)cc1oc3cccc4c(-c5ccc6ccc7cccc8ccc5c6c78)cc2c1c34 ; c1ccc(-c2ccc3ccc4c(-c5cc6c7ccccc7cc7oc8cccc5c8c76)ccc5ccc2c3c54)cc1] 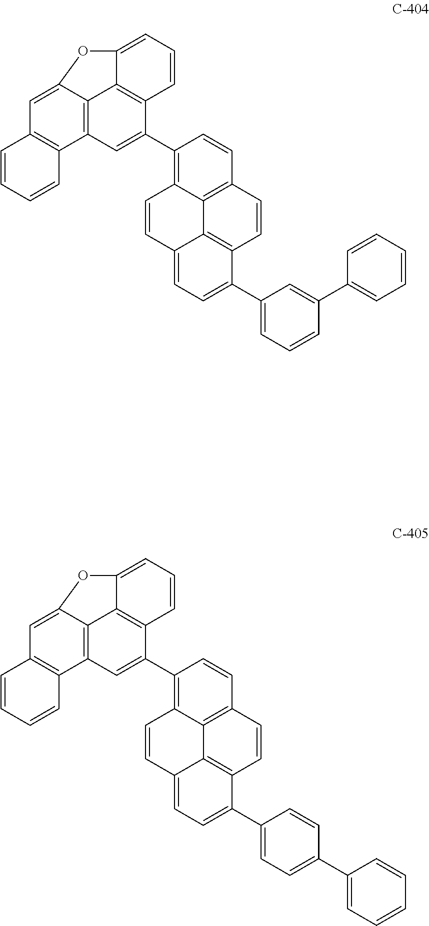 [SMILES: c1ccc(-c2cccc(-c3ccc4ccc5c(-c6cc7c8ccccc8cc8oc9cccc6c9c87)ccc6ccc3c4c65)c2)cc1 ; c1ccc(-c2ccc(-c3ccc4ccc5c(-c6cc7c8ccccc8cc8oc9cccc6c9c87)ccc6ccc3c4c65)cc2)cc1] 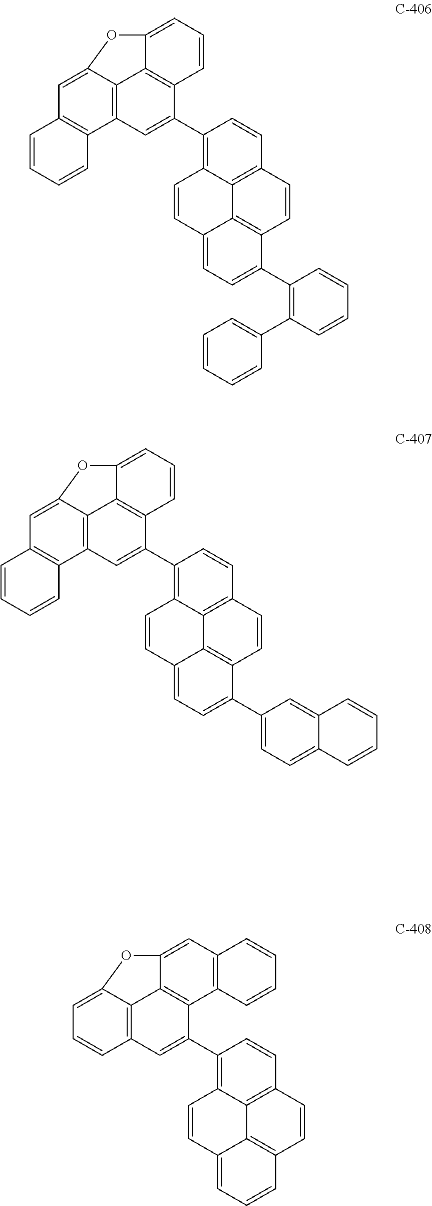 [SMILES: c1ccc(-c2ccccc2-c2ccc3ccc4c(-c5cc6c7ccccc7cc7oc8cccc5c8c76)ccc5ccc2c3c54)cc1 ; c1ccc2cc(-c3ccc4ccc5c(-c6cc7c8ccccc8cc8oc9cccc6c9c87)ccc6ccc3c4c65)ccc2c1 ; c1ccc2c(c1)cc1oc3cccc4cc(-c5ccc6ccc7cccc8ccc5c6c78)c2c1c43] 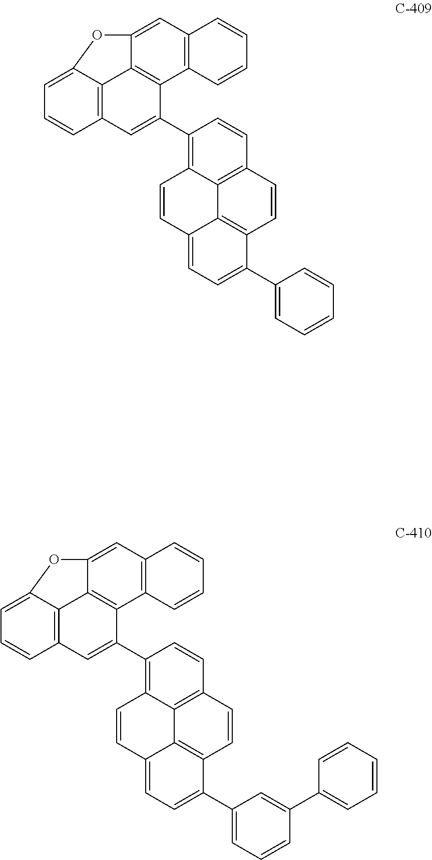 [SMILES: c1ccc(-c2ccc3ccc4c(-c5cc6cccc7oc8cc9ccccc9c5c8c67)ccc5ccc2c3c54)cc1 ; c1ccc(-c2cccc(-c3ccc4ccc5c(-c6cc7cccc8oc9cc%10ccccc%10c6c9c78)ccc6ccc3c4c65)c2)cc1] 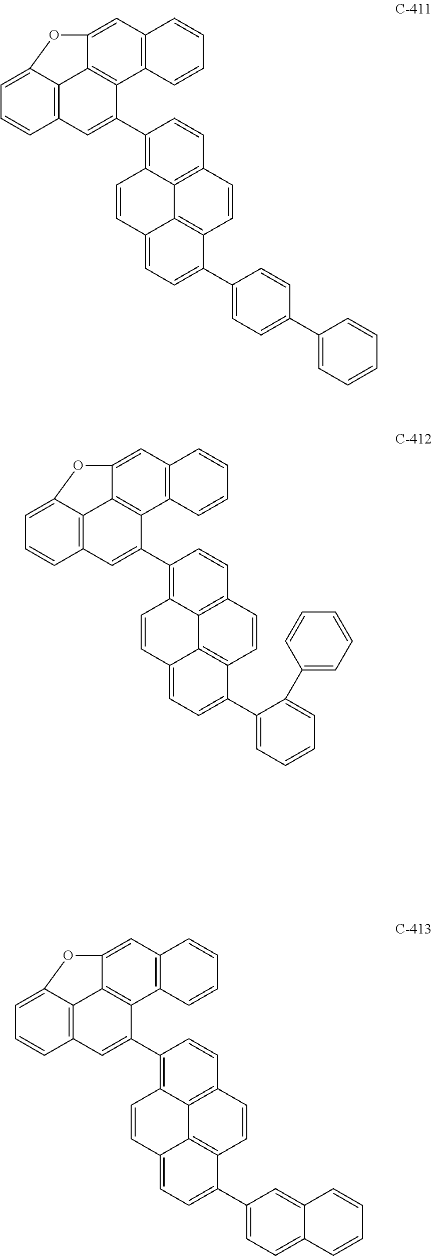 [SMILES: c1ccc(-c2ccc(-c3ccc4ccc5c(-c6cc7cccc8oc9cc%10ccccc%10c6c9c78)ccc6ccc3c4c65)cc2)cc1 ; c1ccc(-c2ccccc2-c2ccc3ccc4c(-c5cc6cccc7oc8cc9ccccc9c5c8c67)ccc5ccc2c3c54)cc1 ; c1ccc2cc(-c3ccc4ccc5c(-c6cc7cccc8oc9cc%10ccccc%10c6c9c78)ccc6ccc3c4c65)ccc2c1] 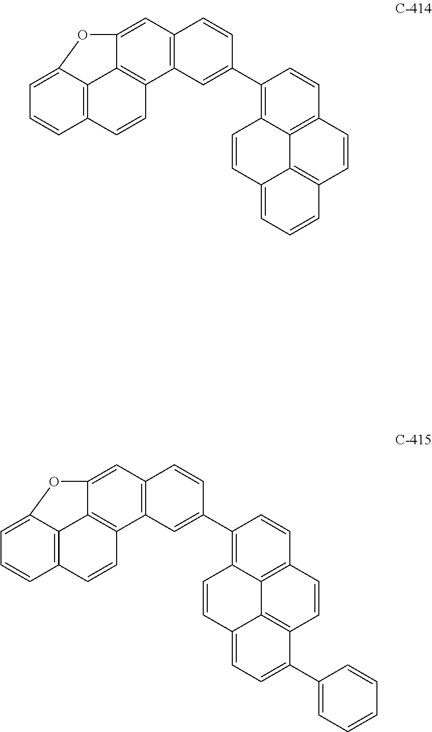 [SMILES: c1cc2ccc3ccc(-c4ccc5cc6oc7cccc8ccc(c5c4)c6c87)c4ccc(c1)c2c34 ; c1ccc(-c2ccc3ccc4c(-c5ccc6cc7oc8cccc9ccc(c6c5)c7c98)ccc5ccc2c3c54)cc1] 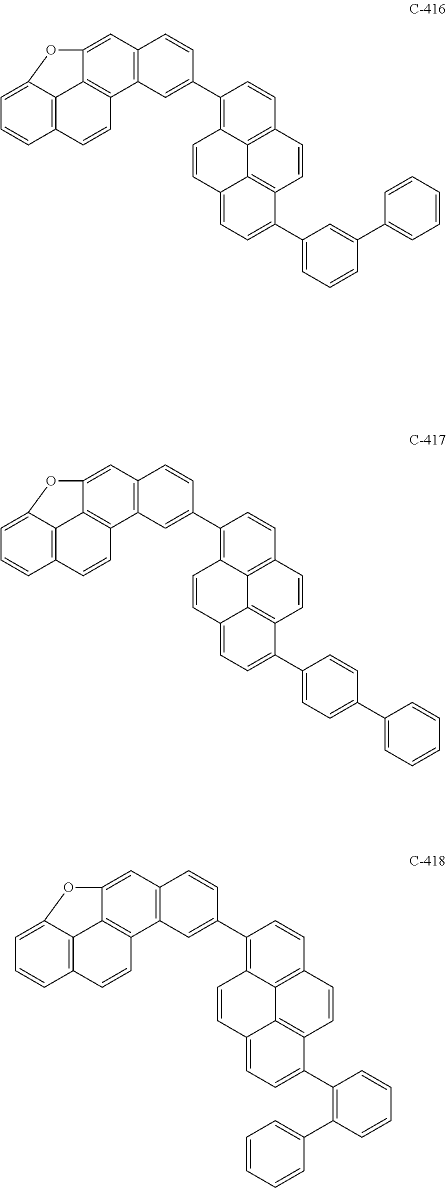 [SMILES: c1ccc(-c2cccc(-c3ccc4ccc5c(-c6ccc7cc8oc9cccc%10ccc(c7c6)c8c%109)ccc6ccc3c4c65)c2)cc1 ; c1ccc(-c2ccc(-c3ccc4ccc5c(-c6ccc7cc8oc9cccc%10ccc(c7c6)c8c%109)ccc6ccc3c4c65)cc2)cc1 ; c1ccc(-c2ccccc2-c2ccc3ccc4c(-c5ccc6cc7oc8cccc9ccc(c6c5)c7c98)ccc5ccc2c3c54)cc1] 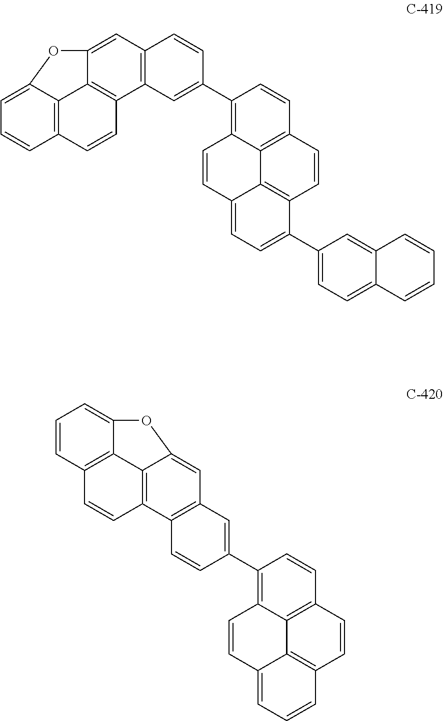 [SMILES: c1ccc2cc(-c3ccc4ccc5c(-c6ccc7cc8oc9cccc%10ccc(c7c6)c8c%109)ccc6ccc3c4c65)ccc2c1 ; c1cc2ccc3ccc(-c4ccc5c(c4)cc4oc6cccc7ccc5c4c76)c4ccc(c1)c2c34] 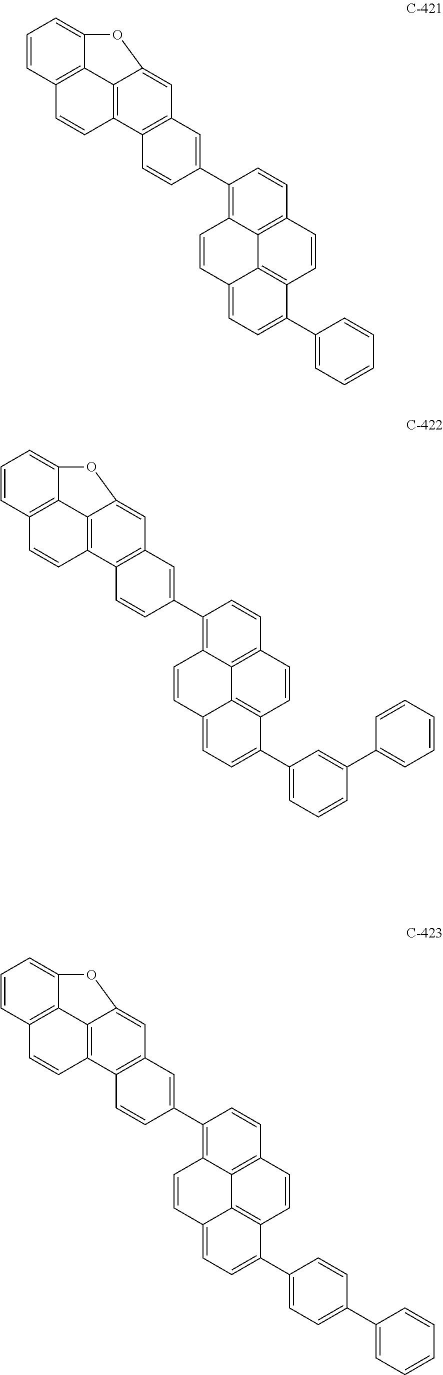 [SMILES: c1ccc(-c2ccc3ccc4c(-c5ccc6c(c5)cc5oc7cccc8ccc6c5c87)ccc5ccc2c3c54)cc1 ; c1ccc(-c2cccc(-c3ccc4ccc5c(-c6ccc7c(c6)cc6oc8cccc9ccc7c6c98)ccc6ccc3c4c65)c2)cc1 ; c1ccc(-c2ccc(-c3ccc4ccc5c(-c6ccc7c(c6)cc6oc8cccc9ccc7c6c98)ccc6ccc3c4c65)cc2)cc1] 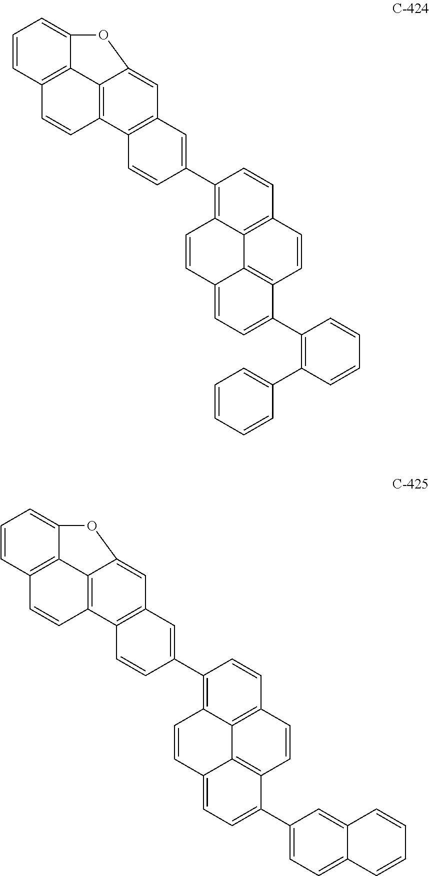 [SMILES: c1ccc(-c2ccccc2-c2ccc3ccc4c(-c5ccc6c(c5)cc5oc7cccc8ccc6c5c87)ccc5ccc2c3c54)cc1 ; c1ccc2cc(-c3ccc4ccc5c(-c6ccc7c(c6)cc6oc8cccc9ccc7c6c98)ccc6ccc3c4c65)ccc2c1] 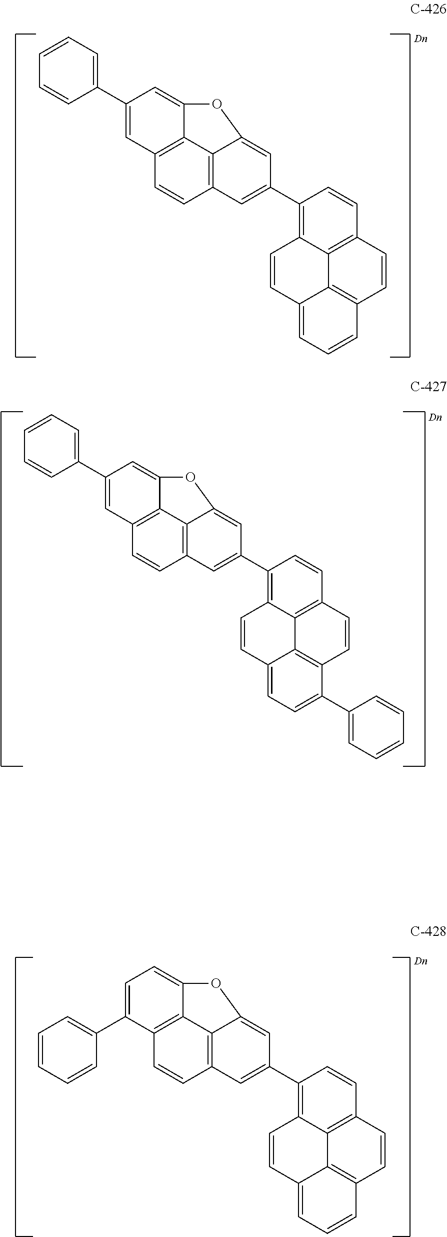 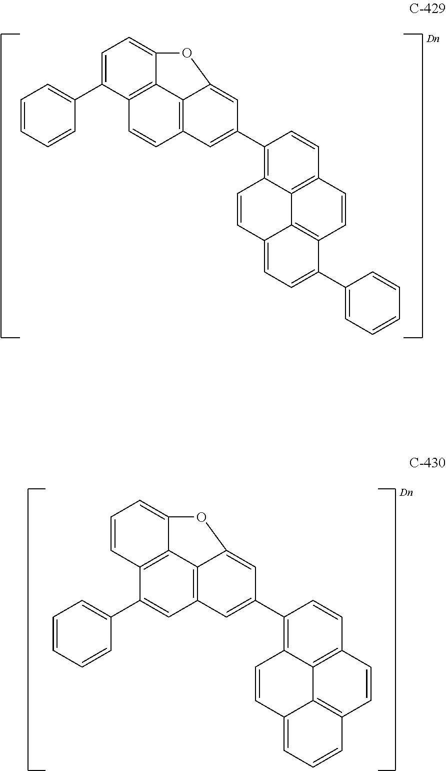 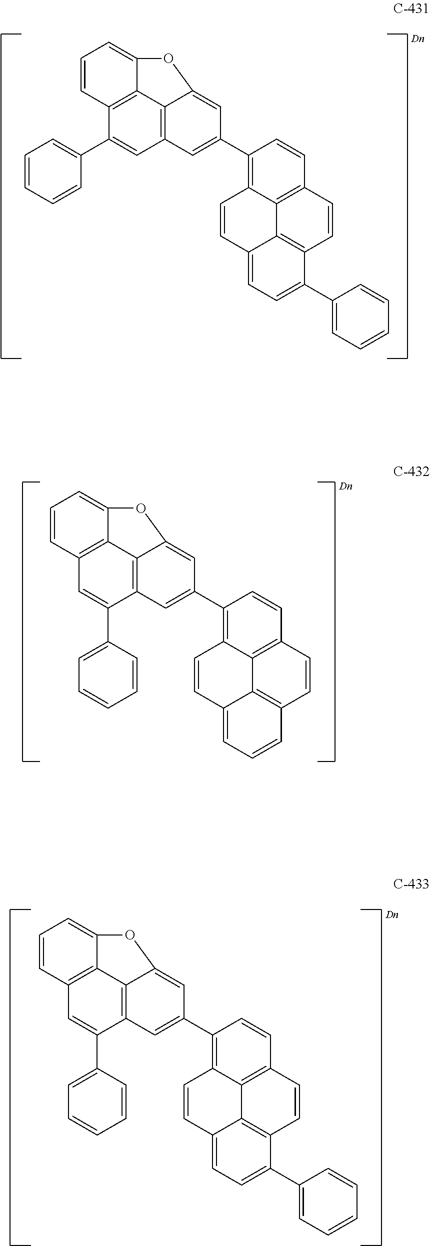 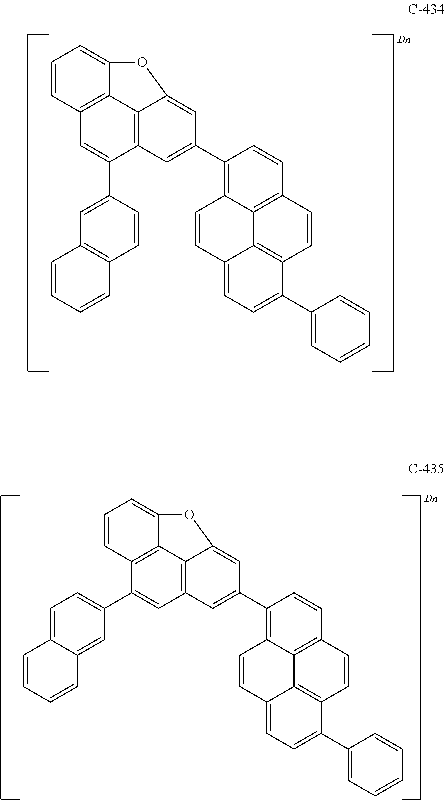 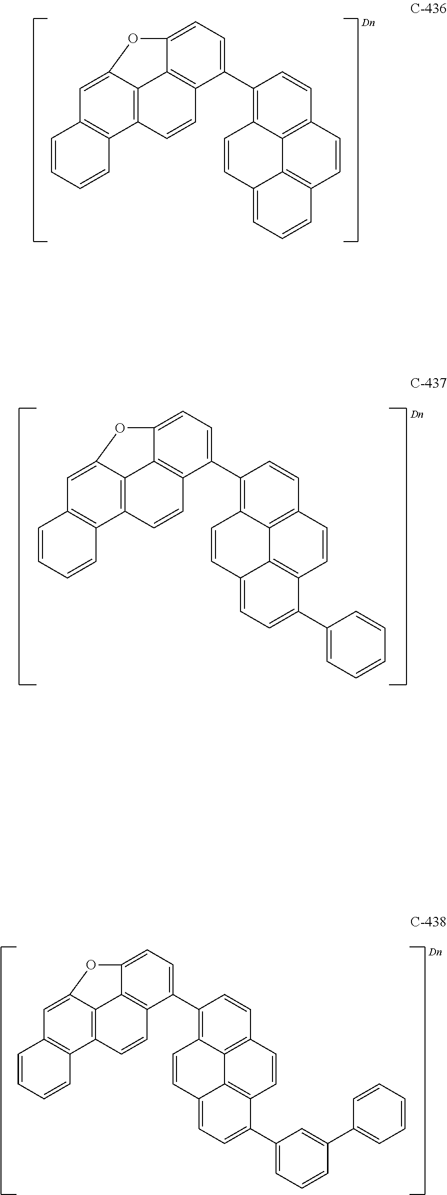 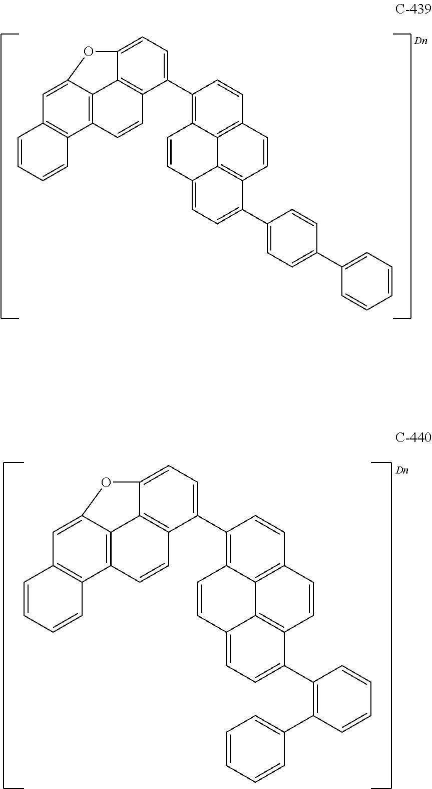 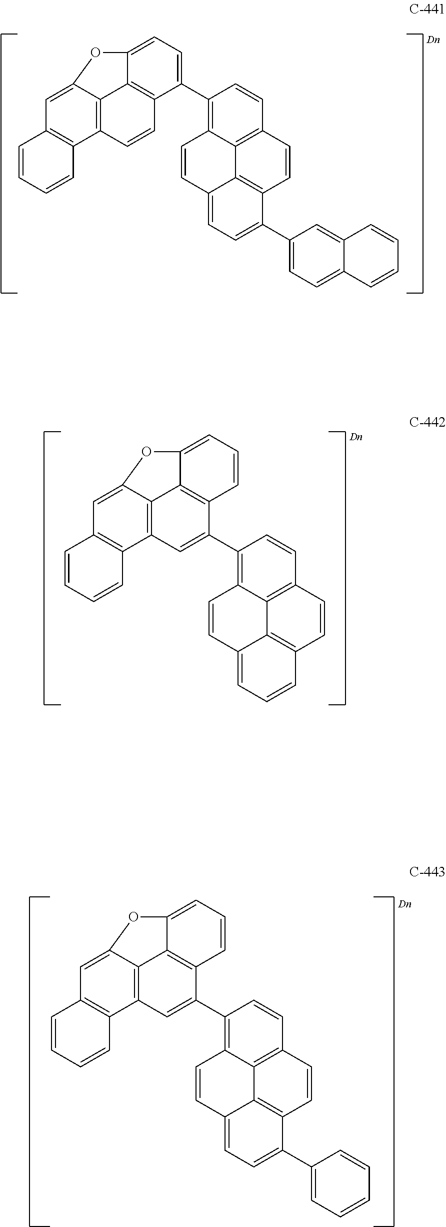 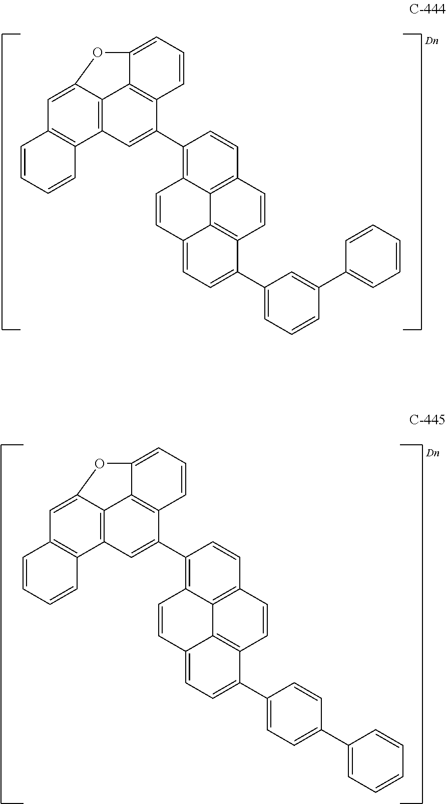 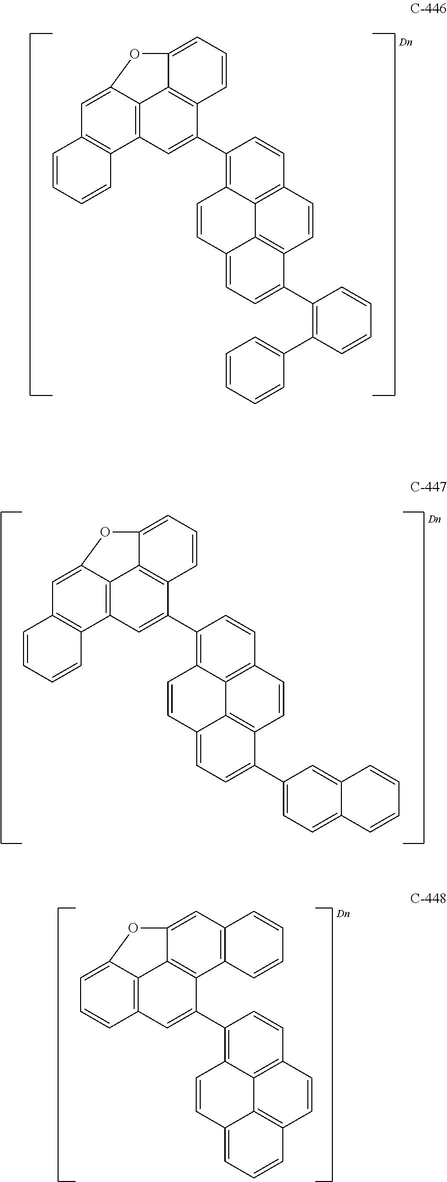 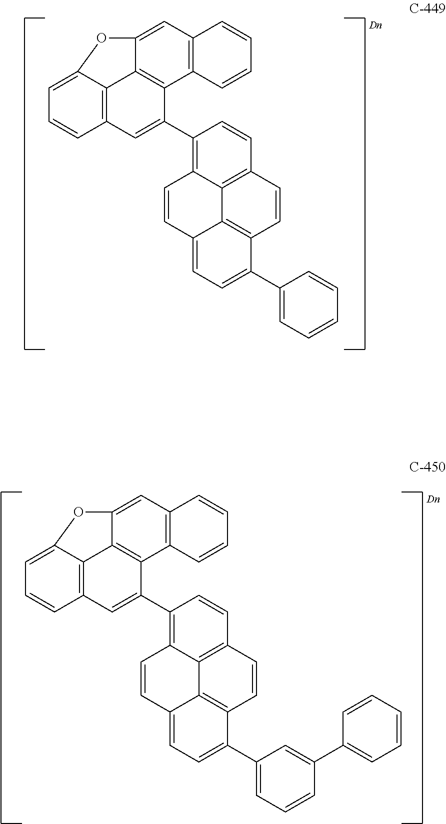 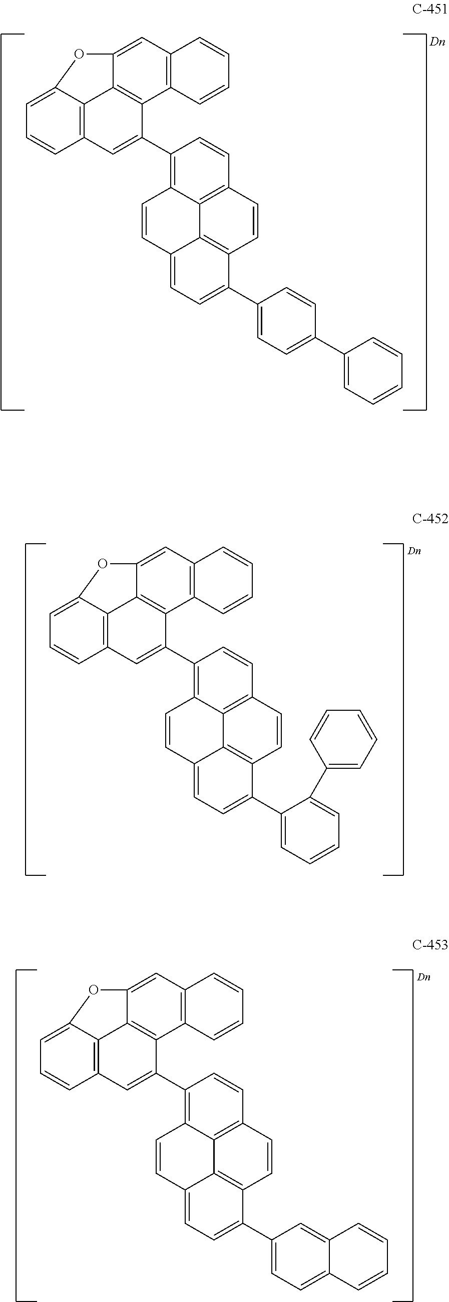 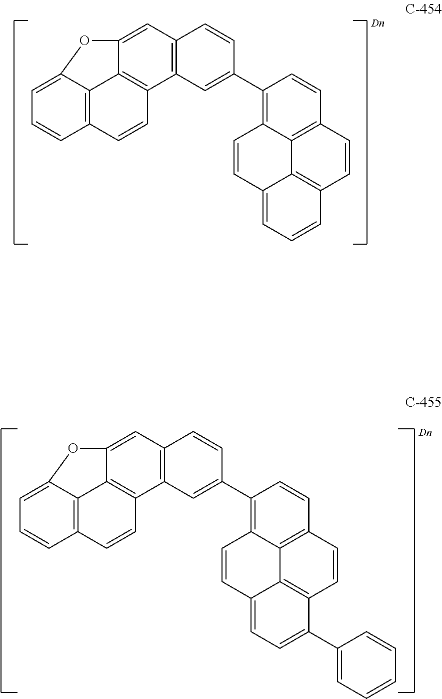 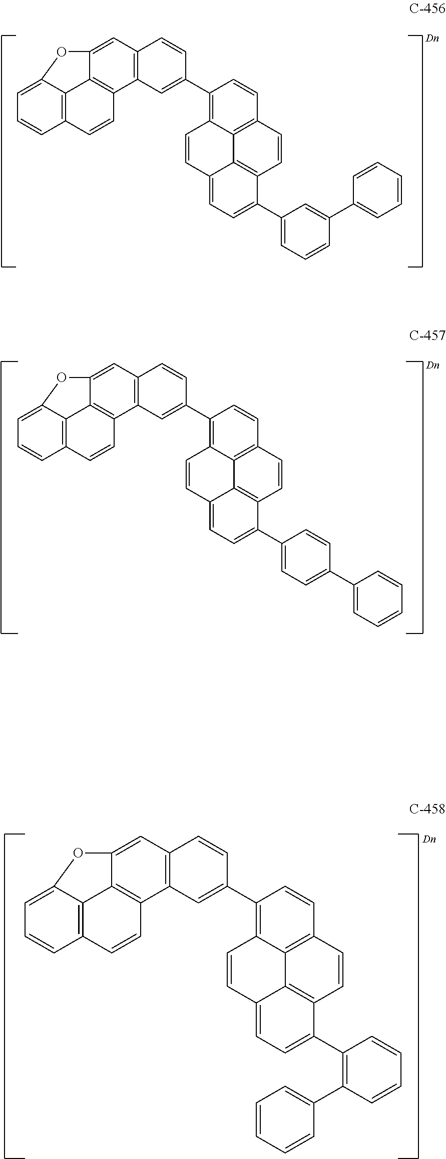 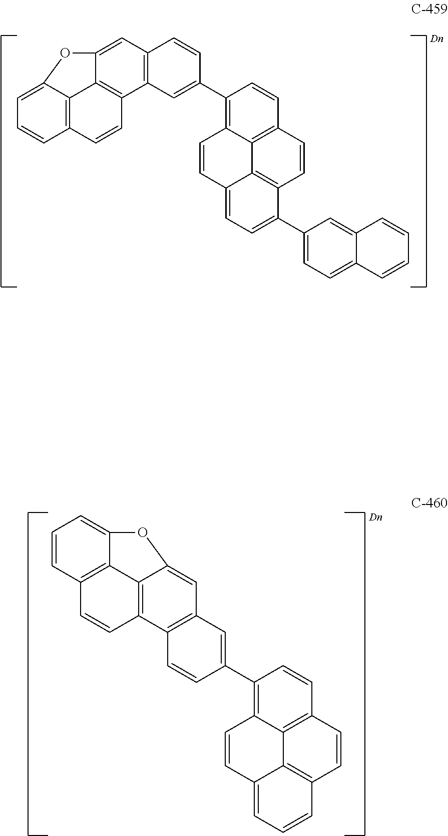 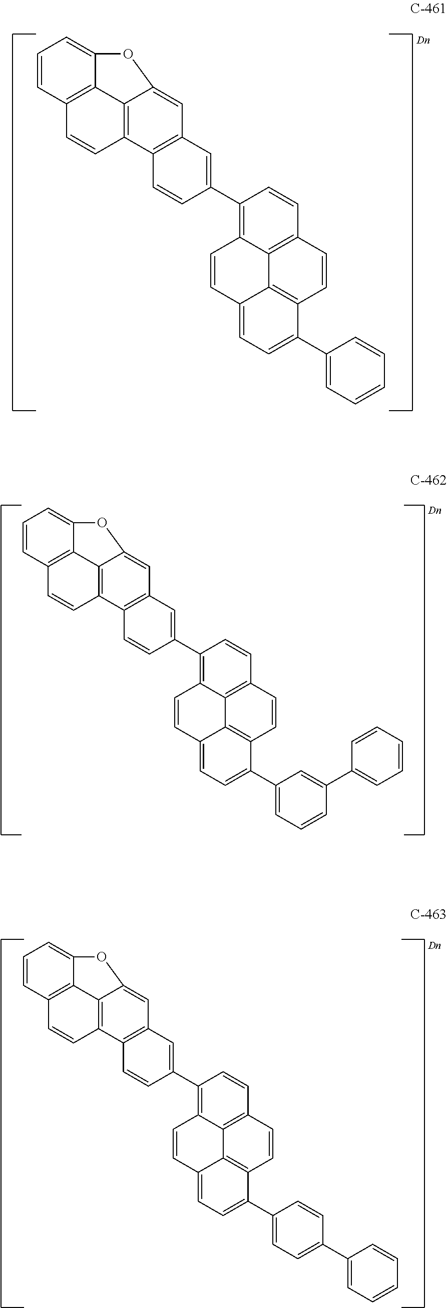 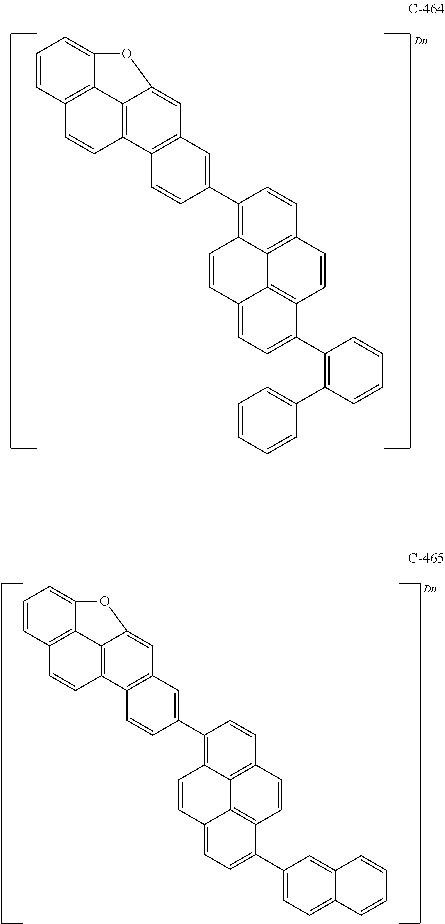 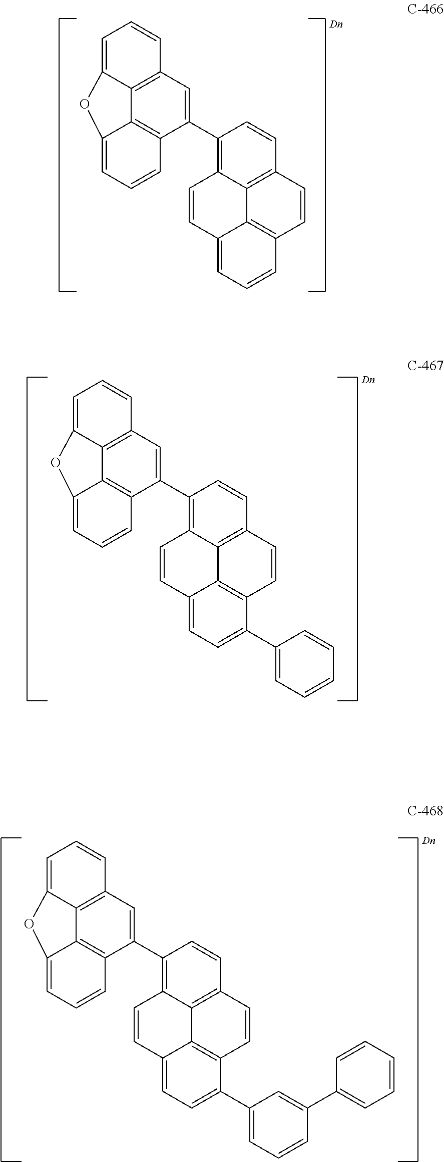 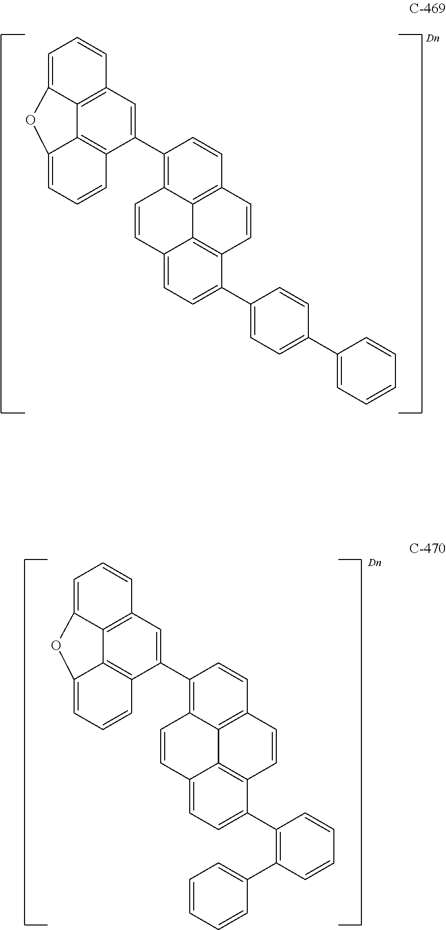 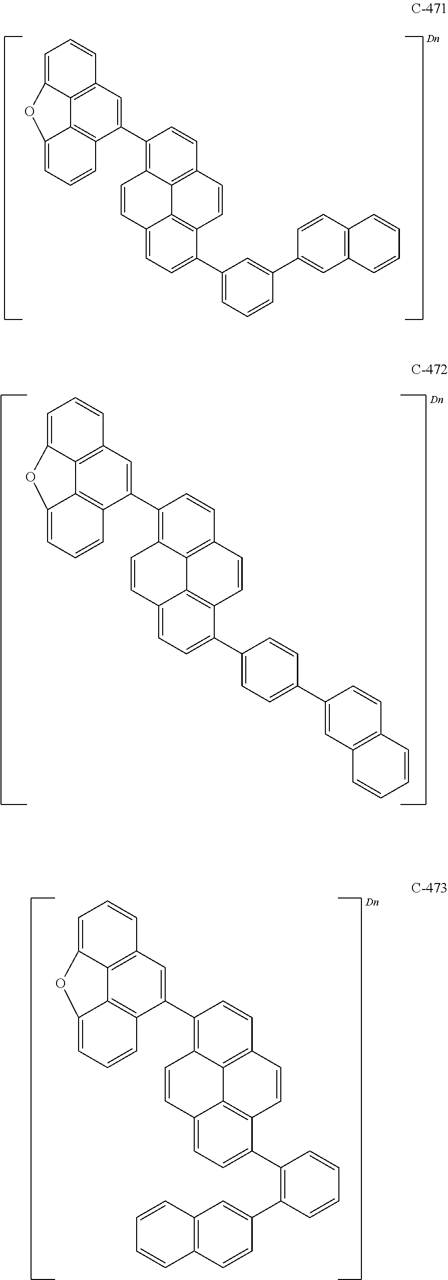 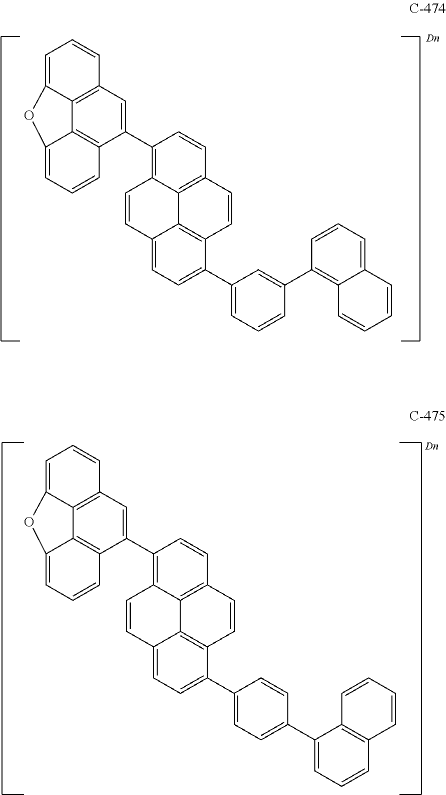 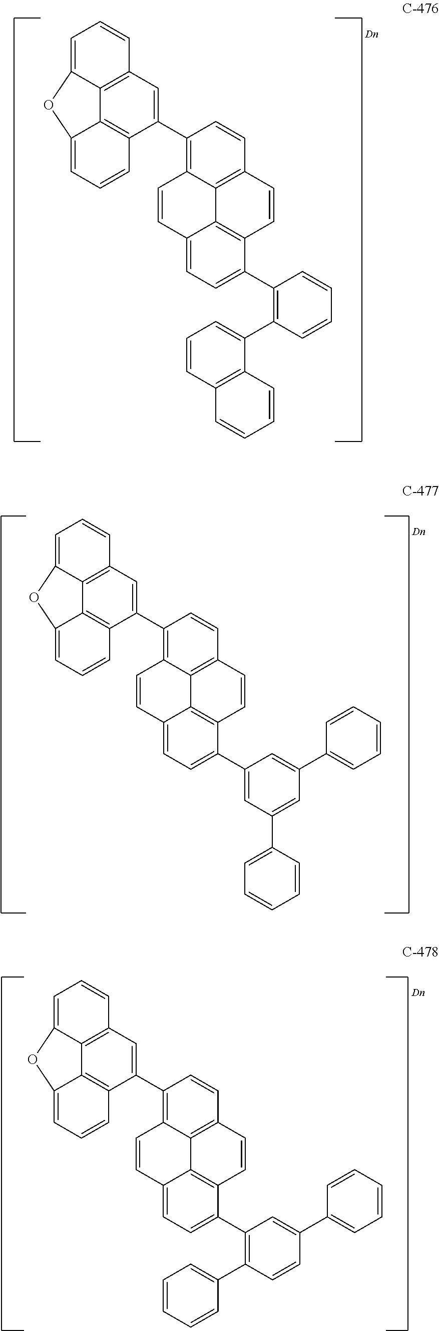 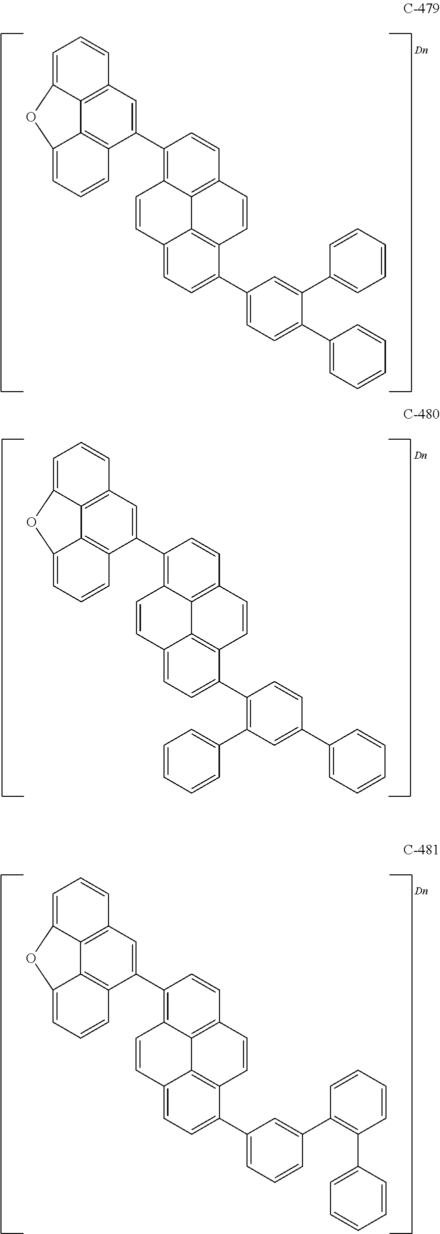 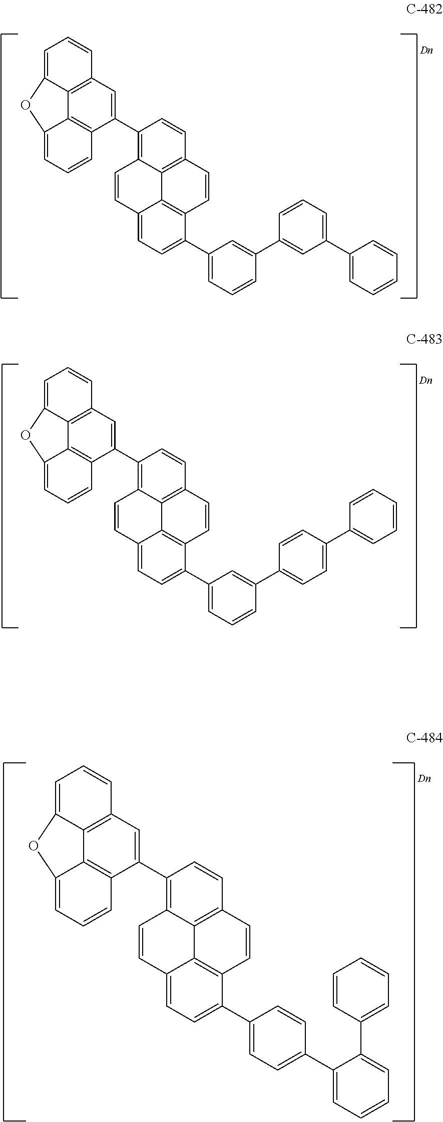 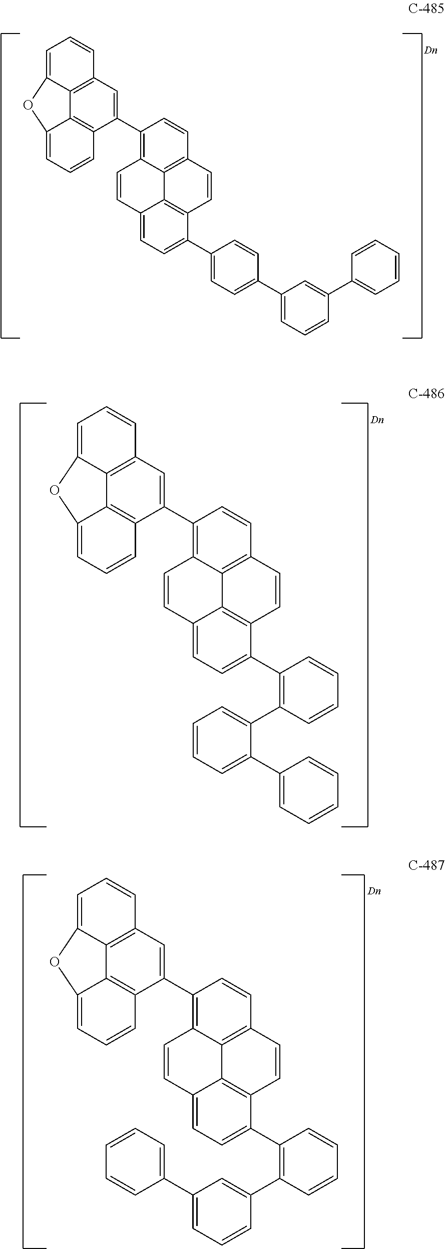 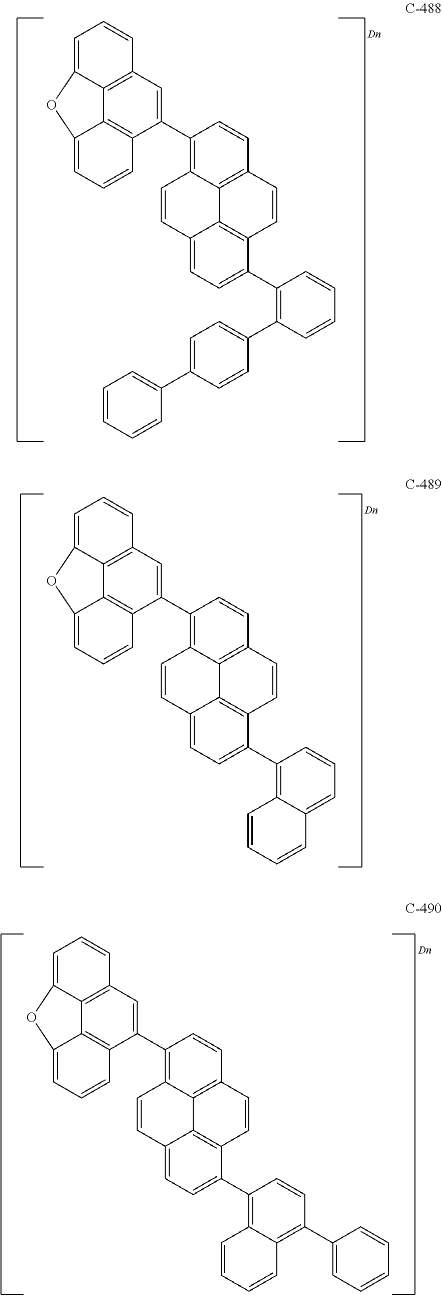 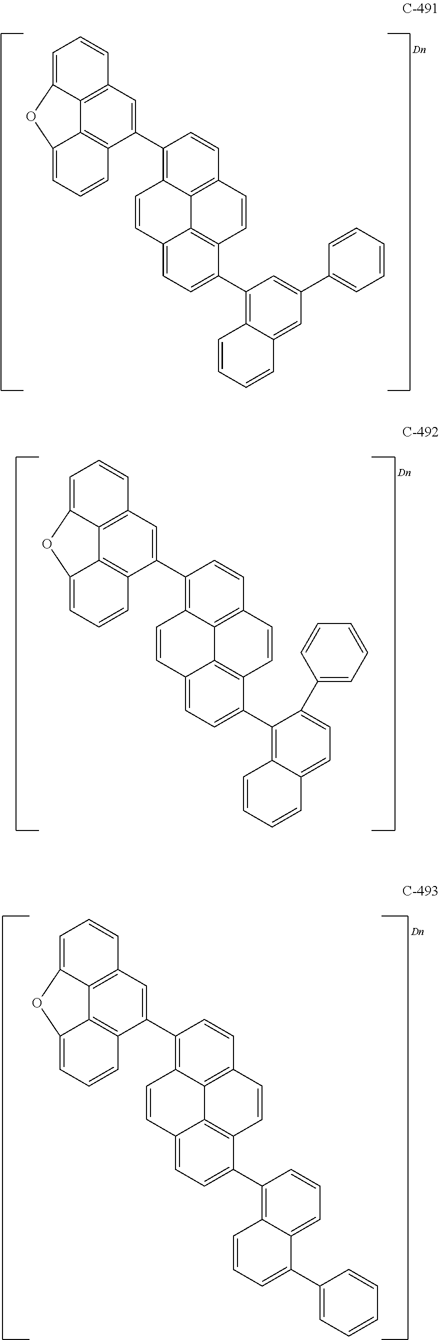 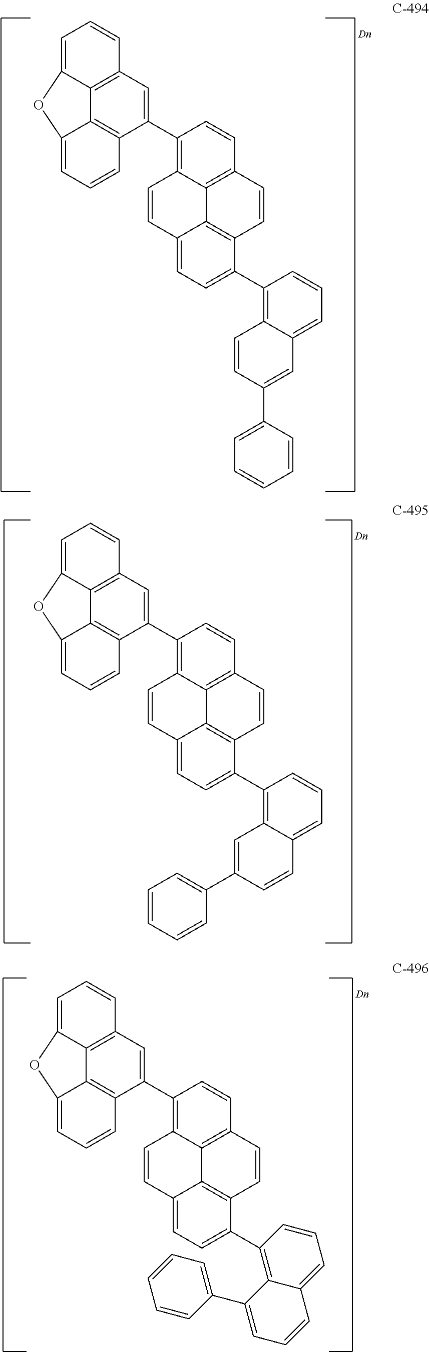 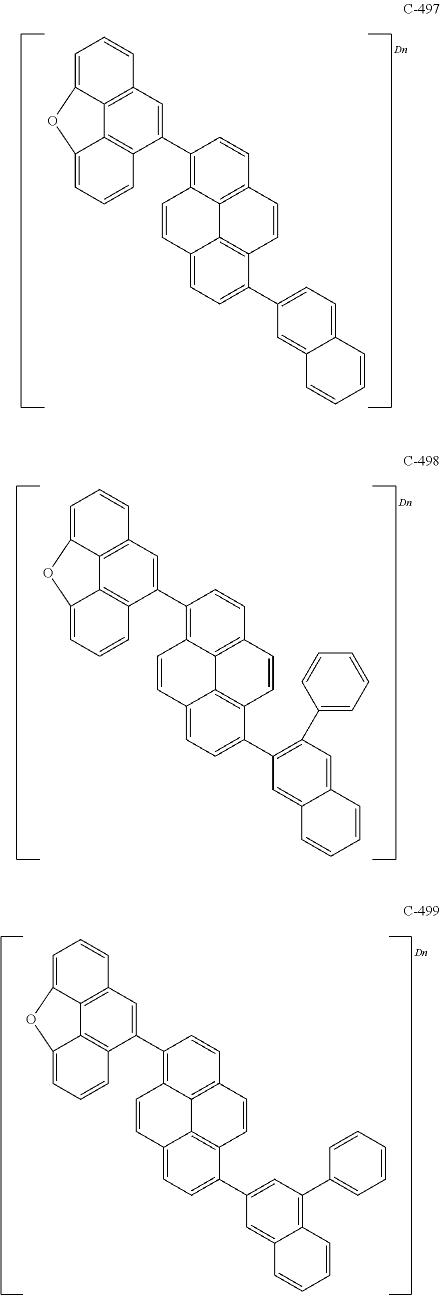 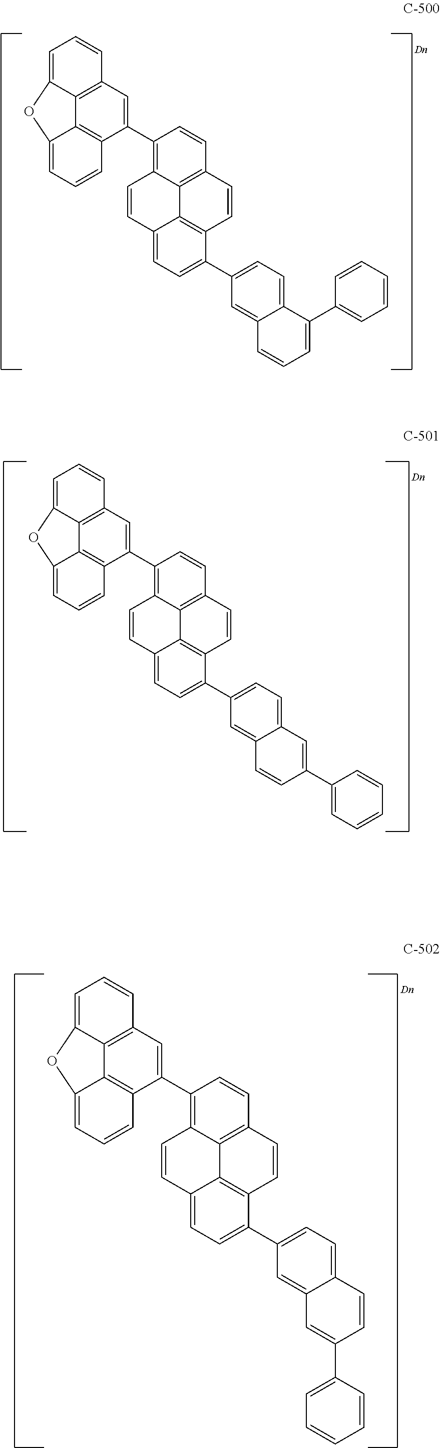 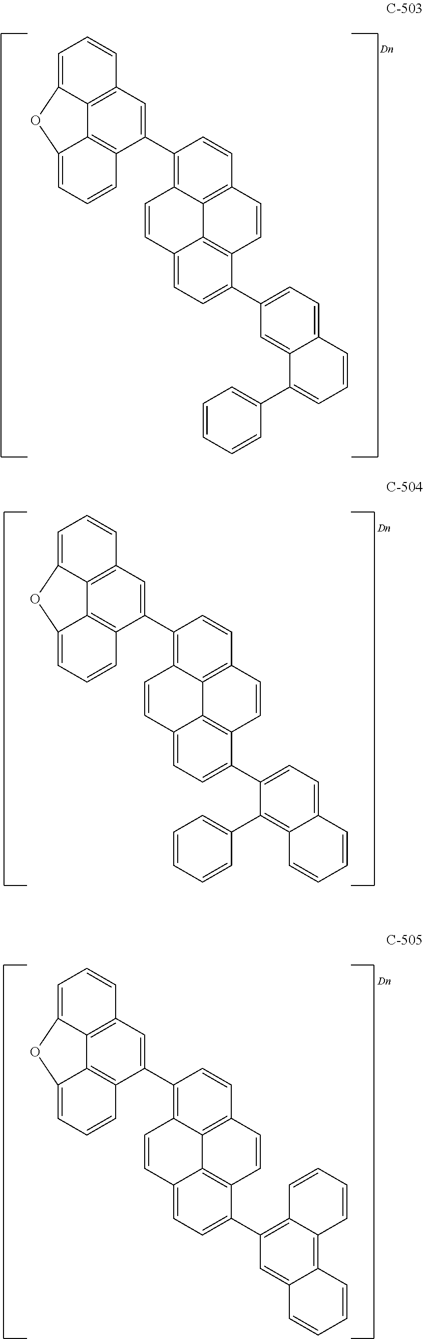 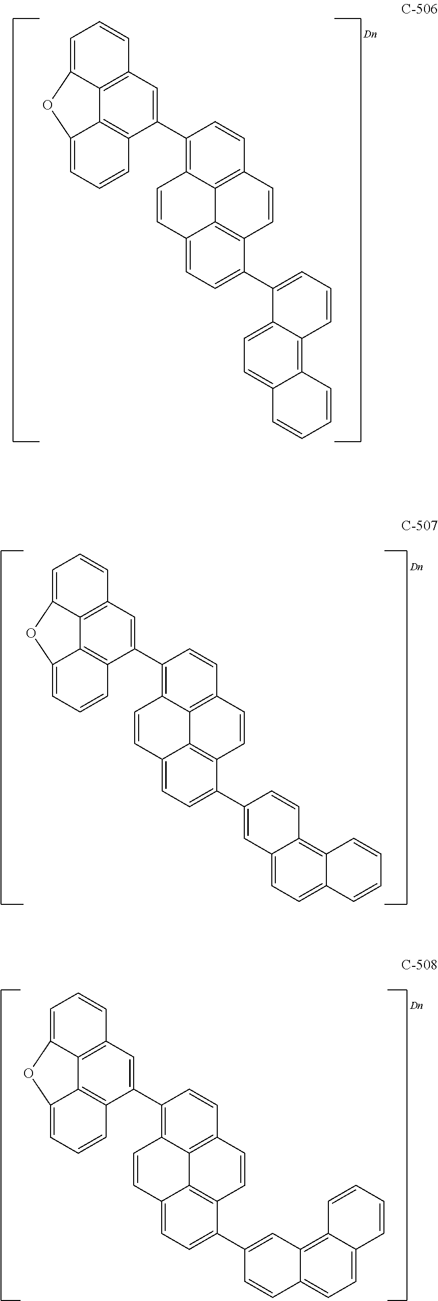 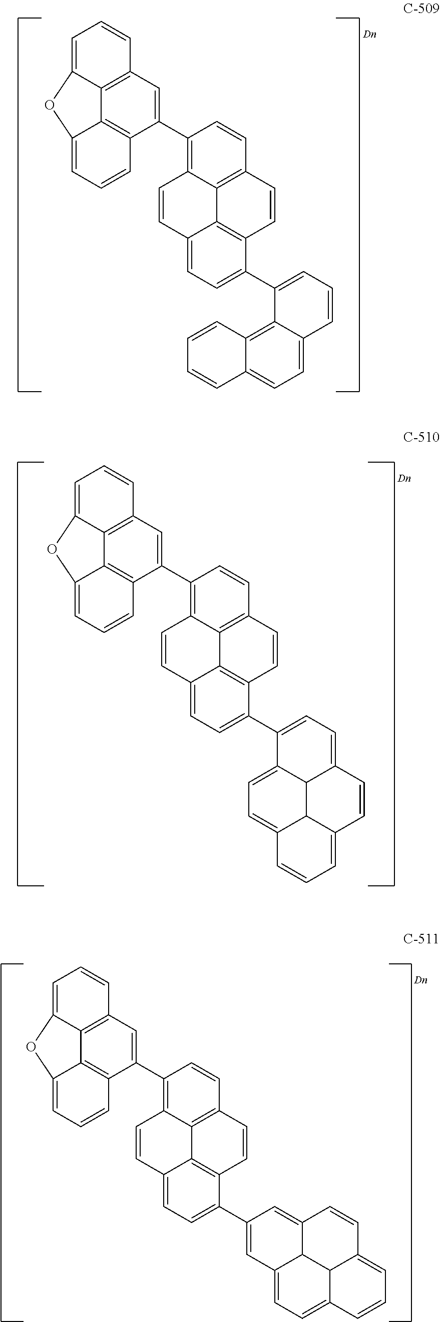 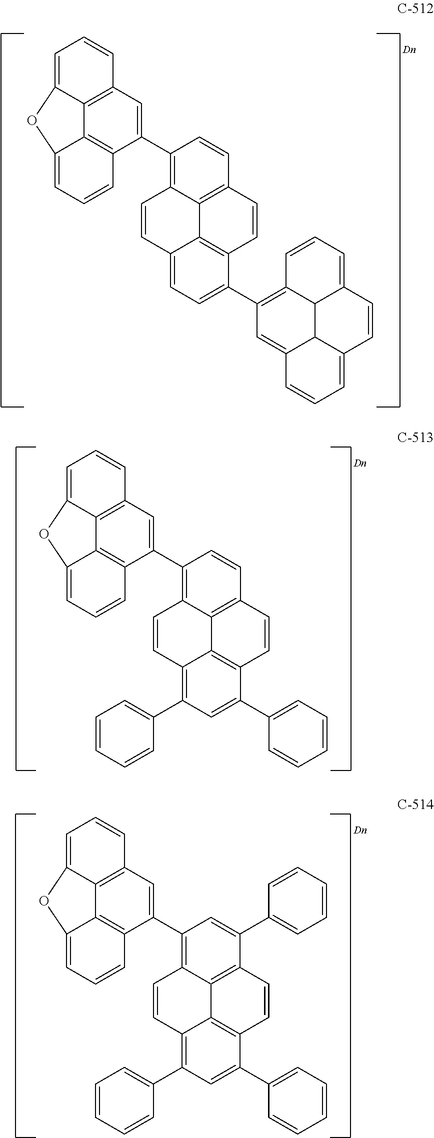 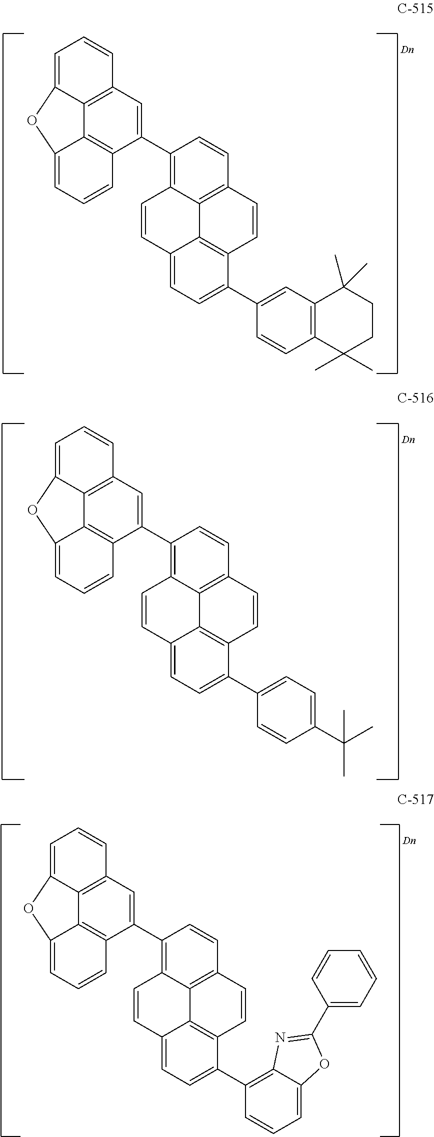 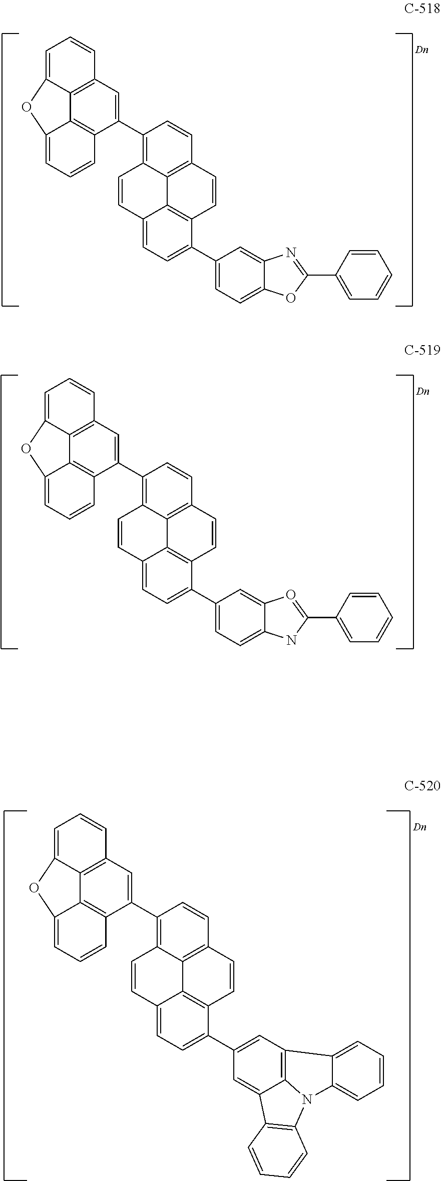 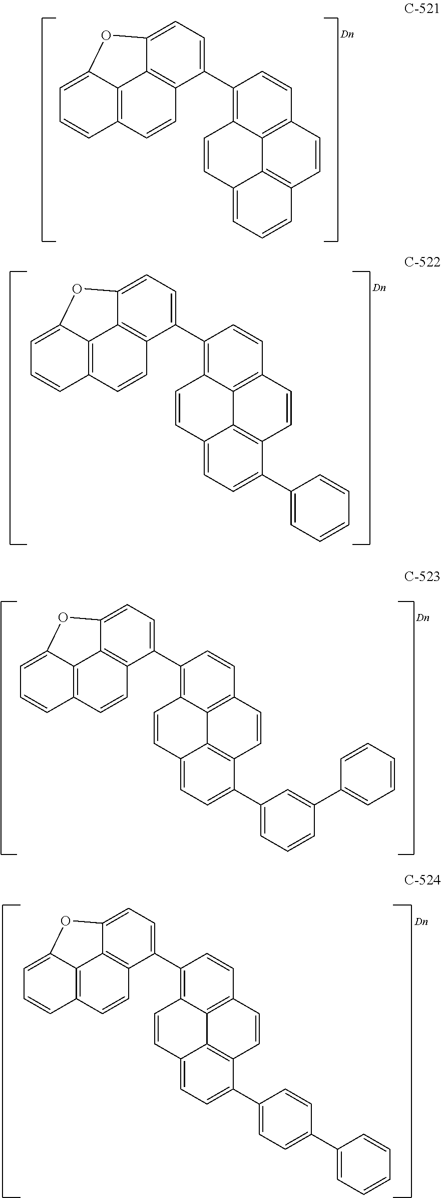 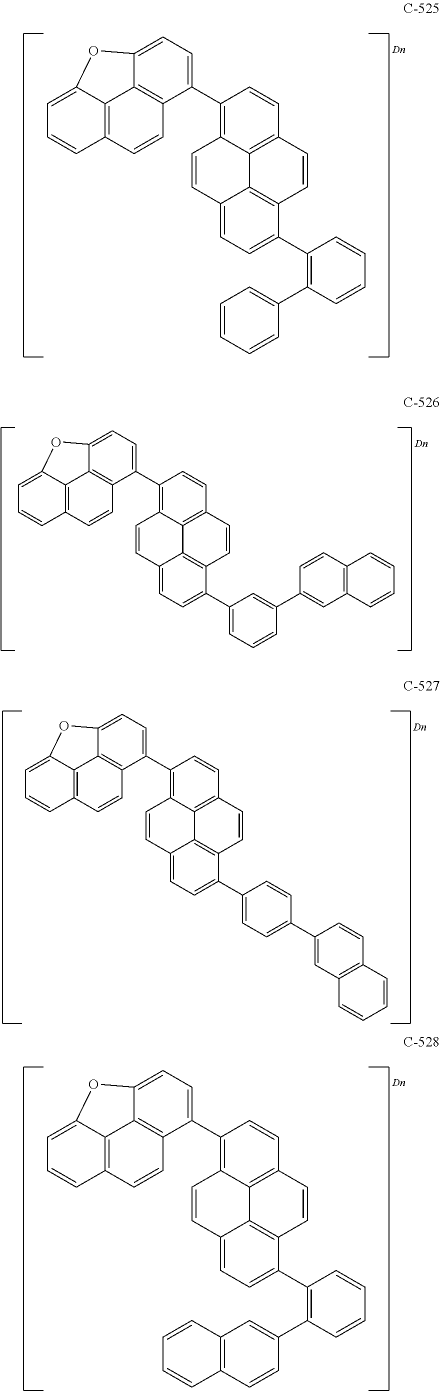 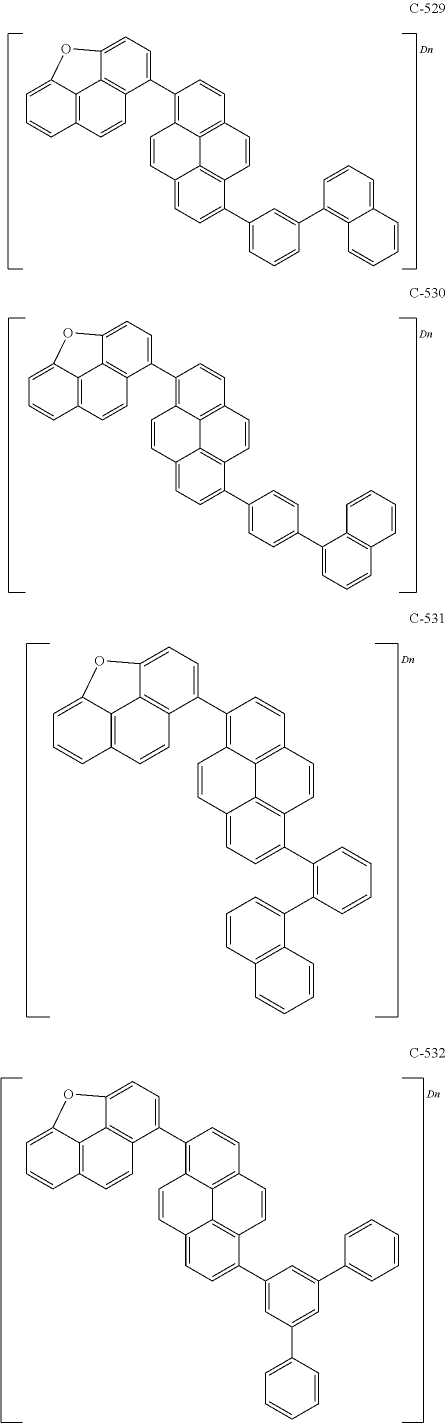 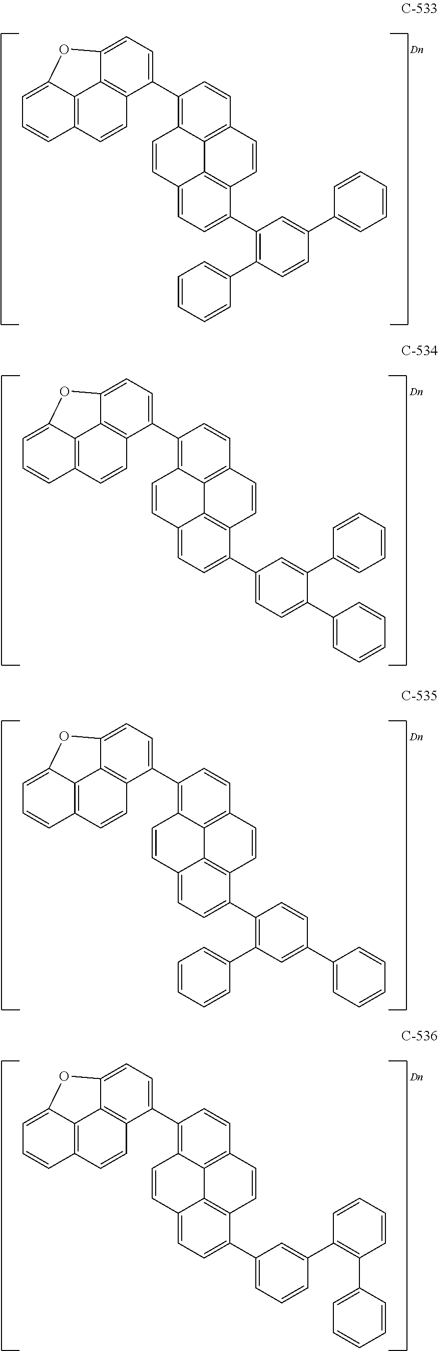 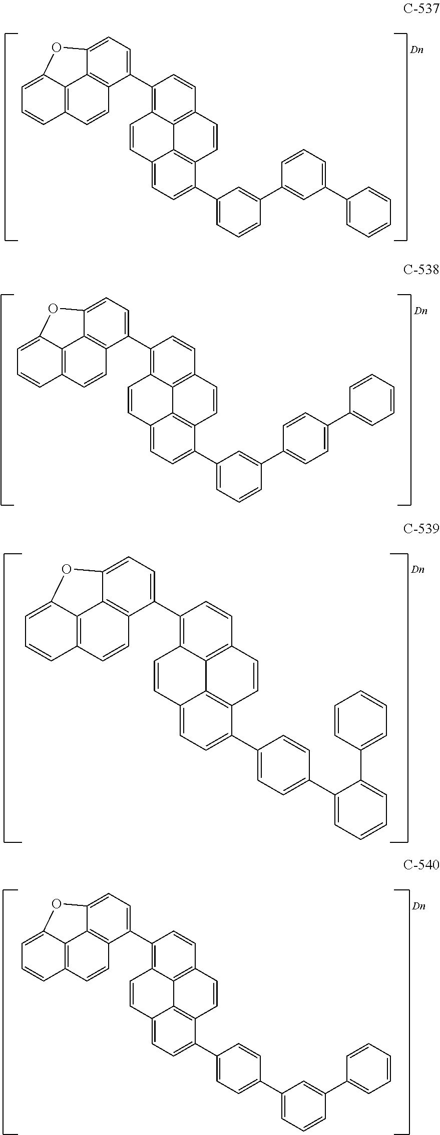 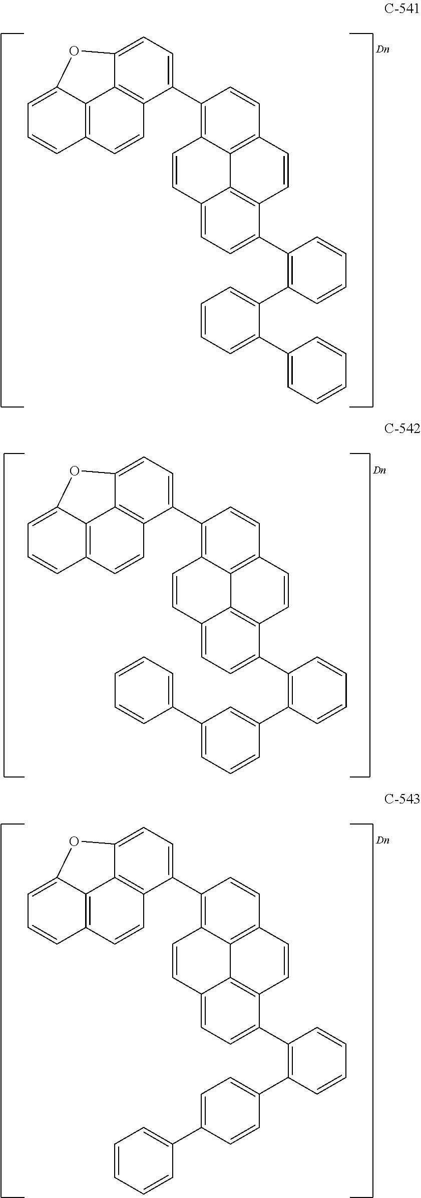 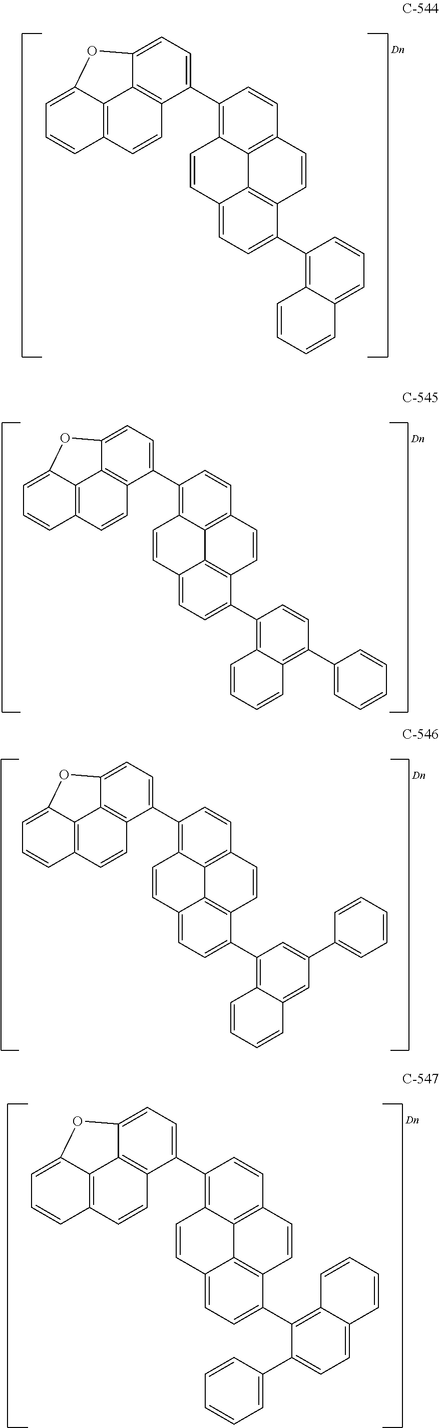 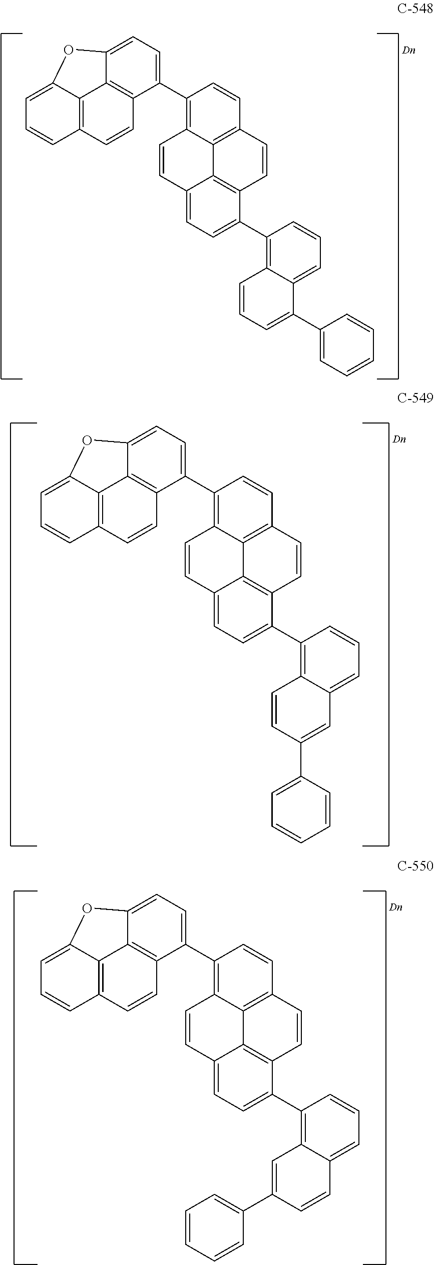 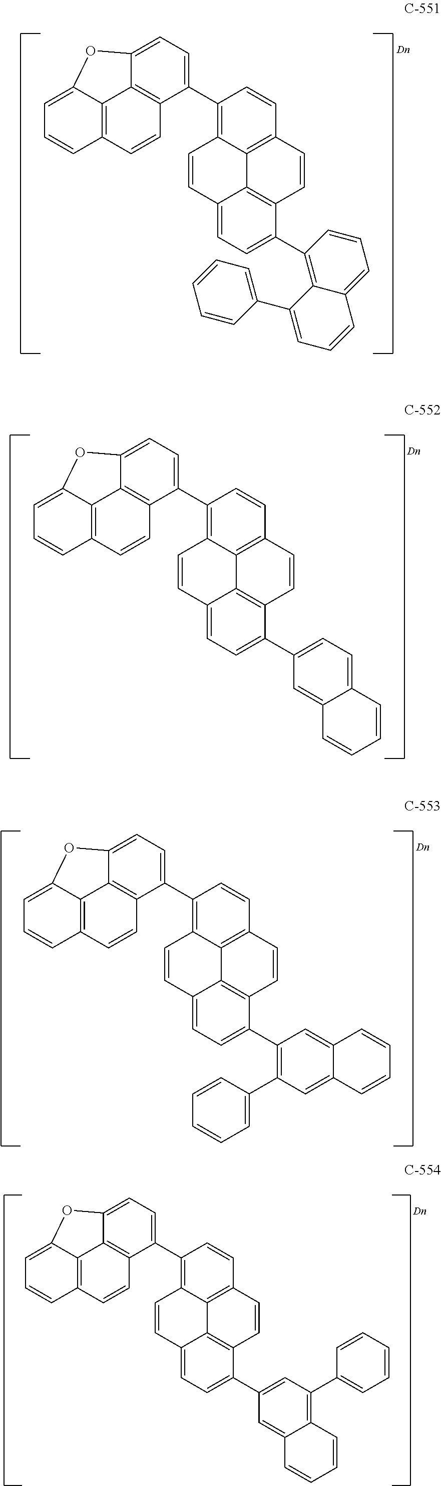 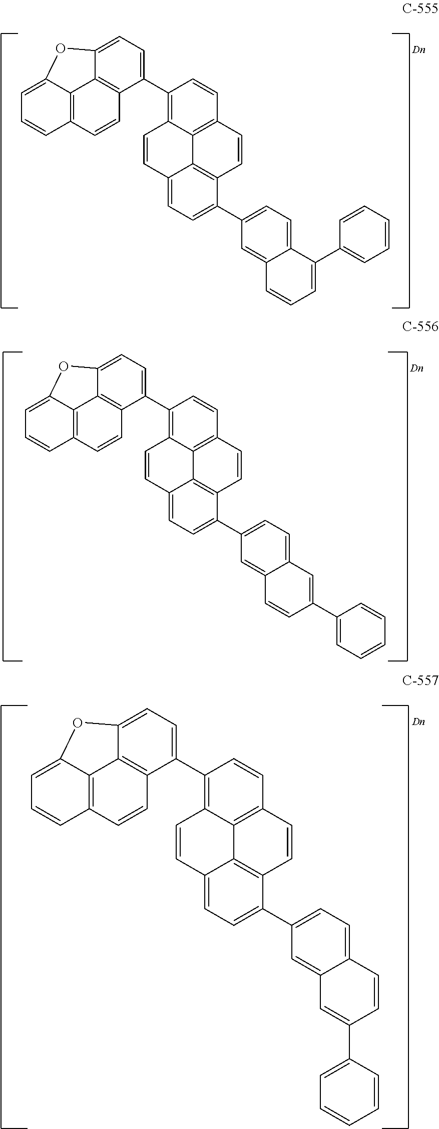 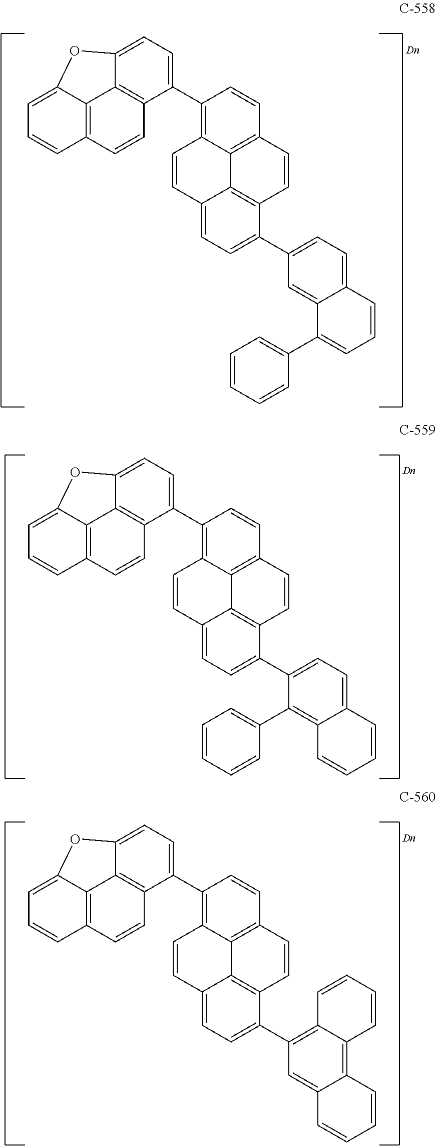 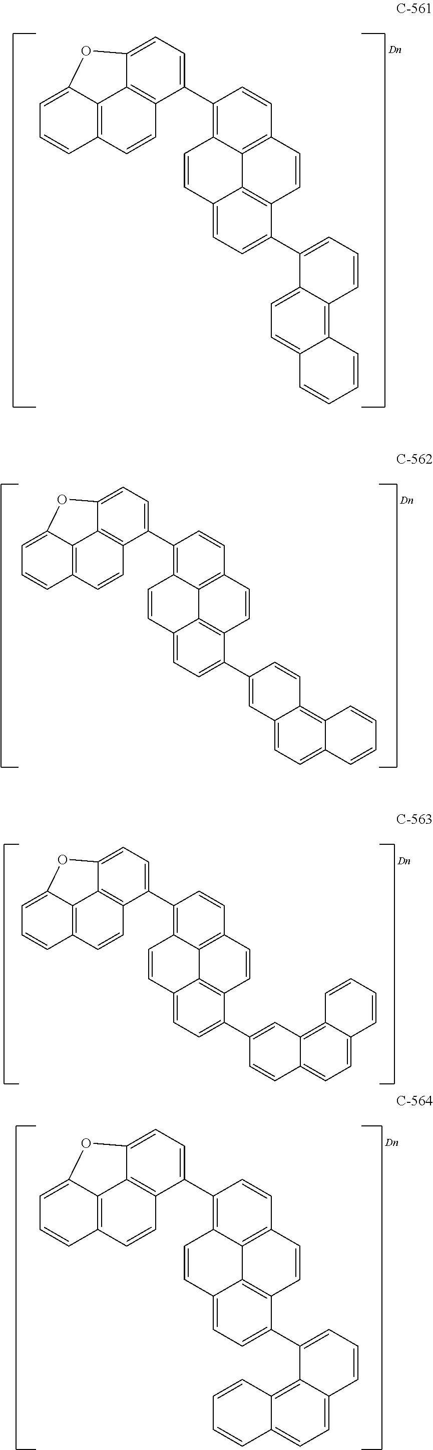 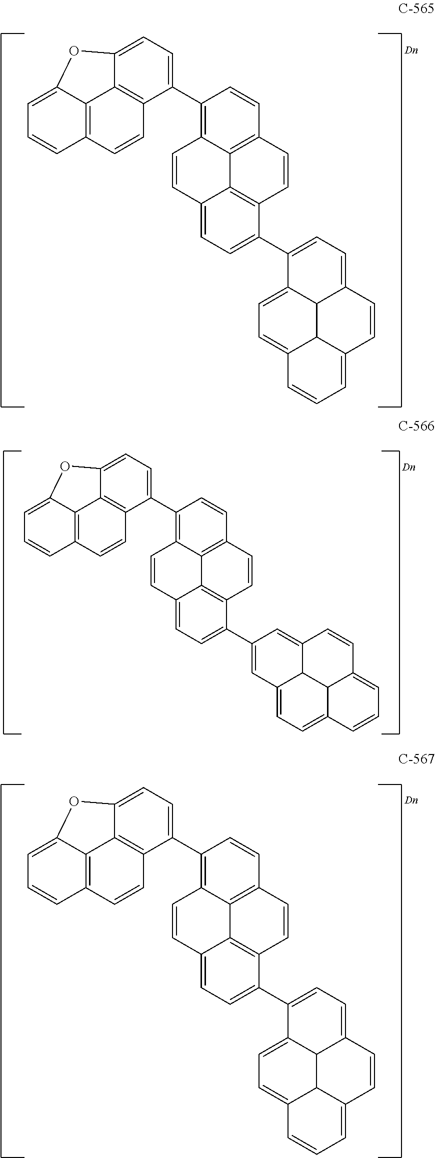 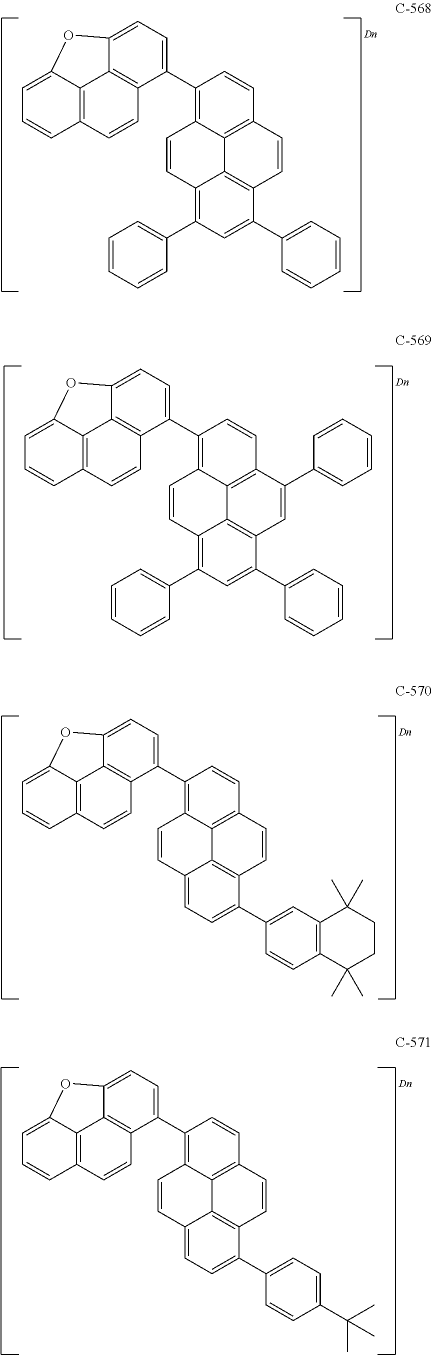 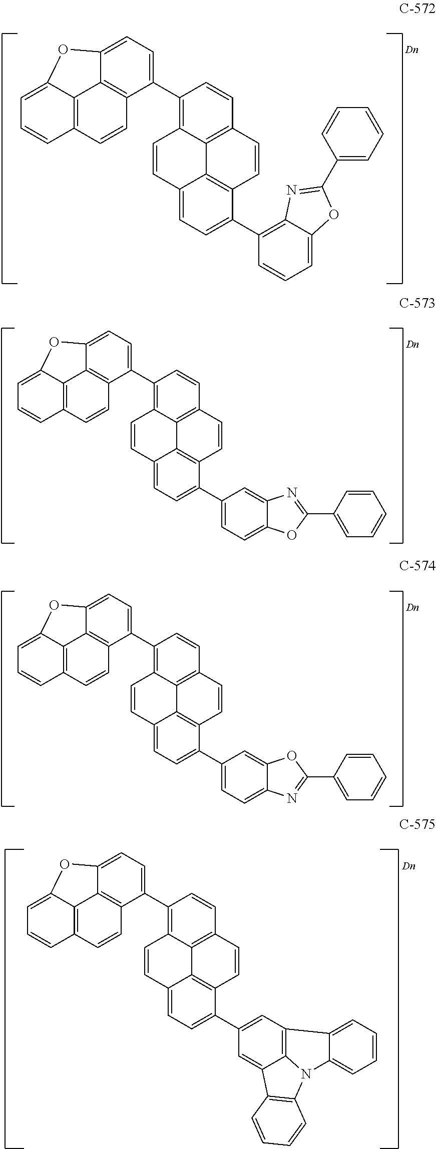 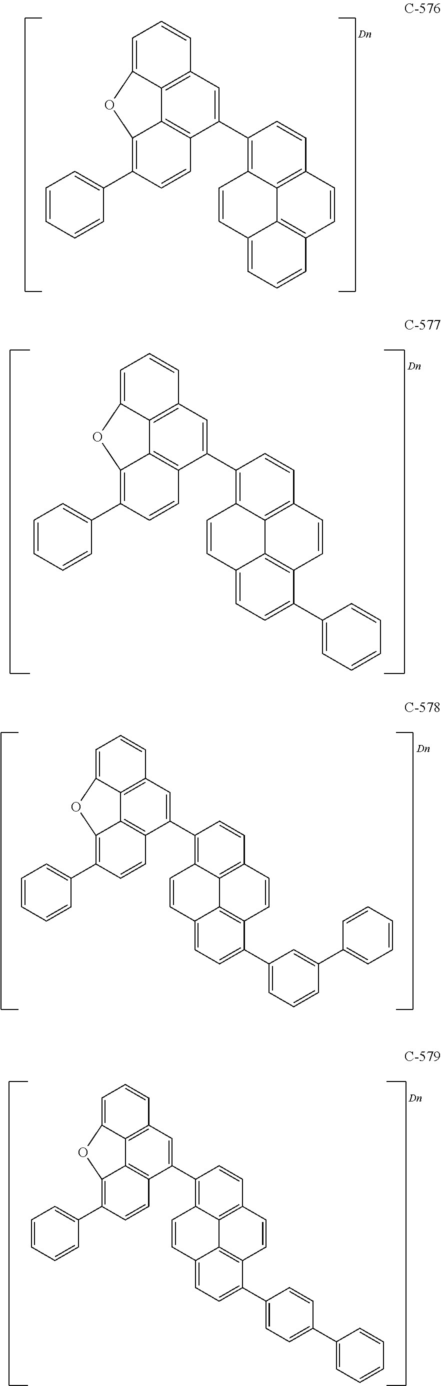 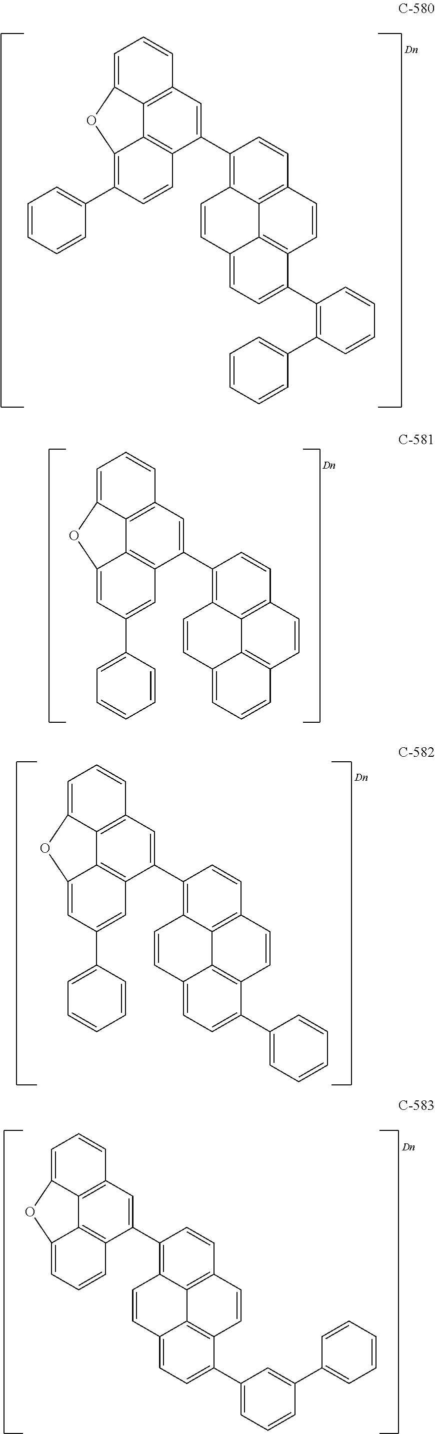 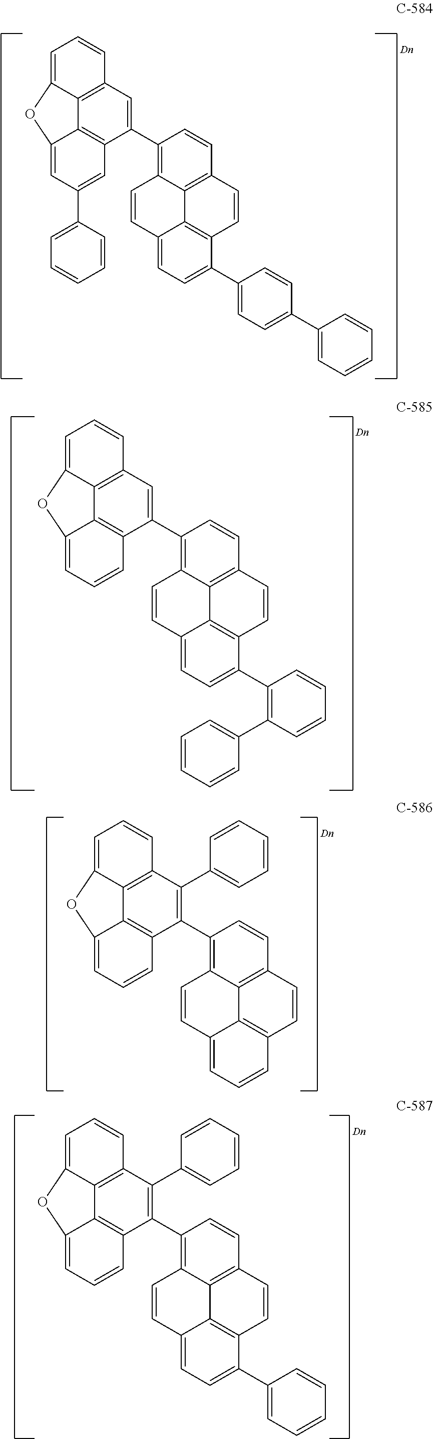 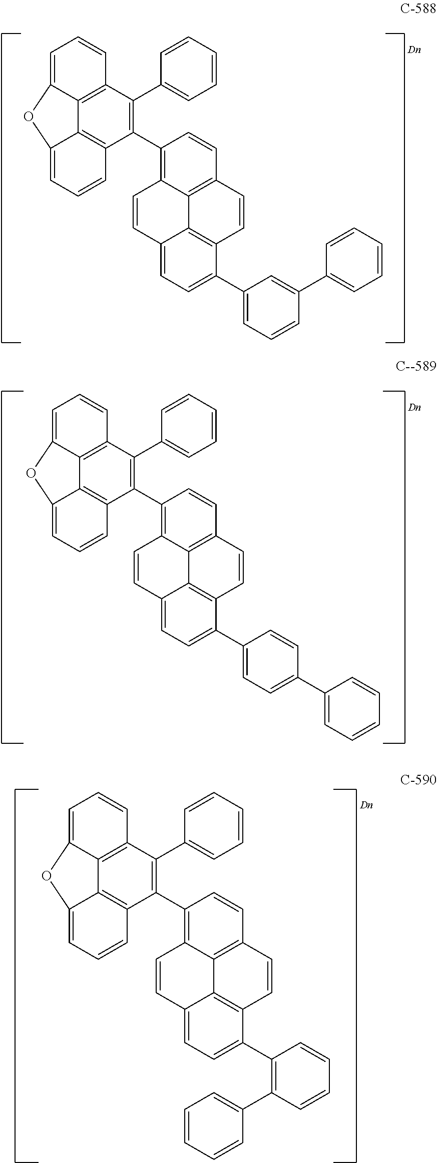 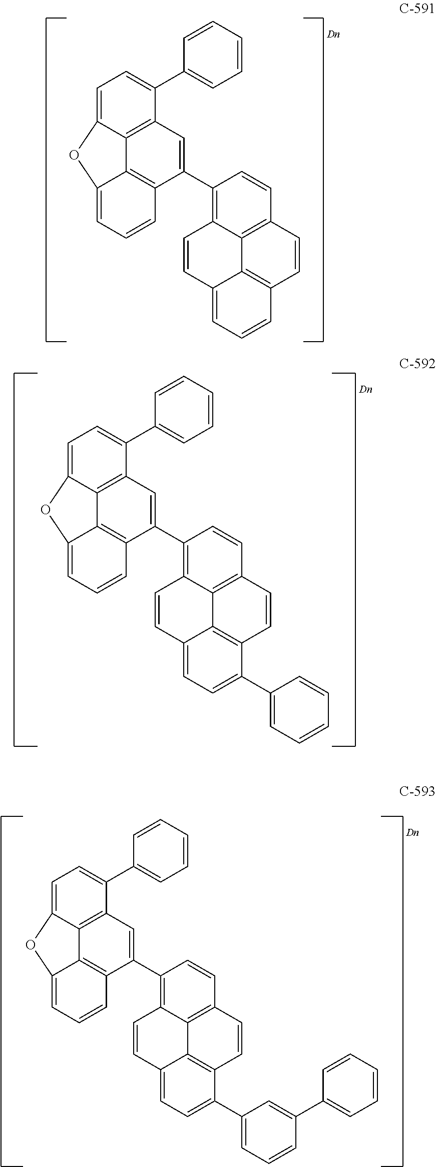 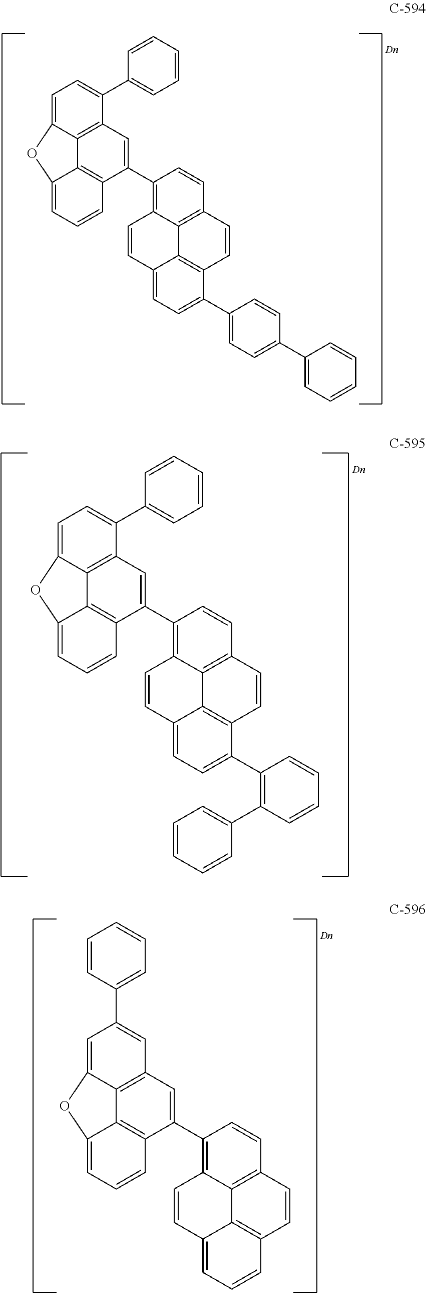 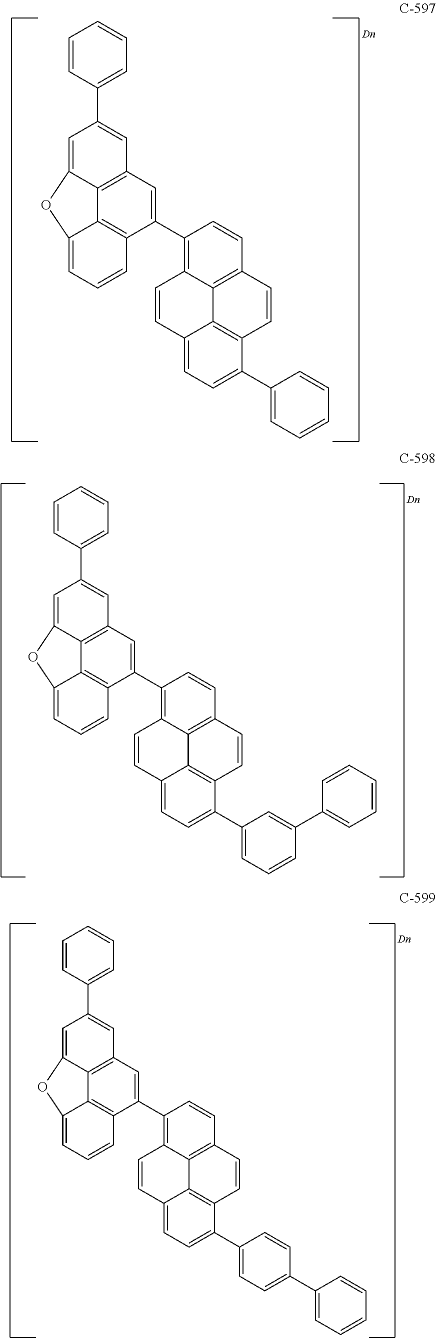 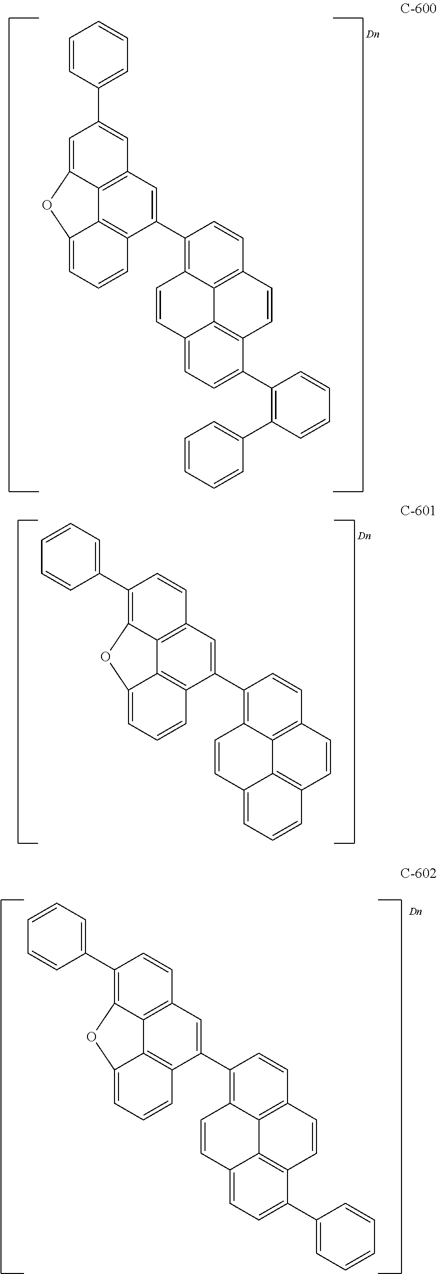 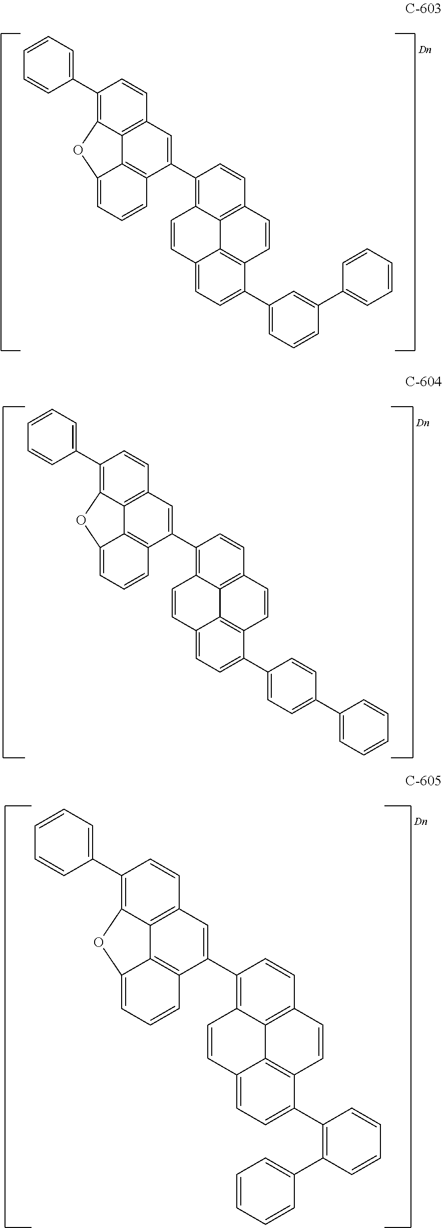 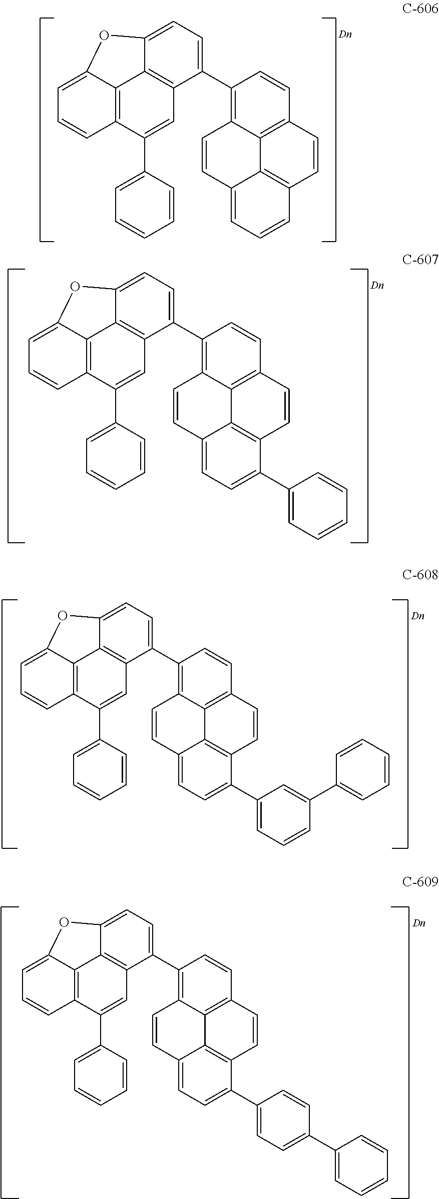 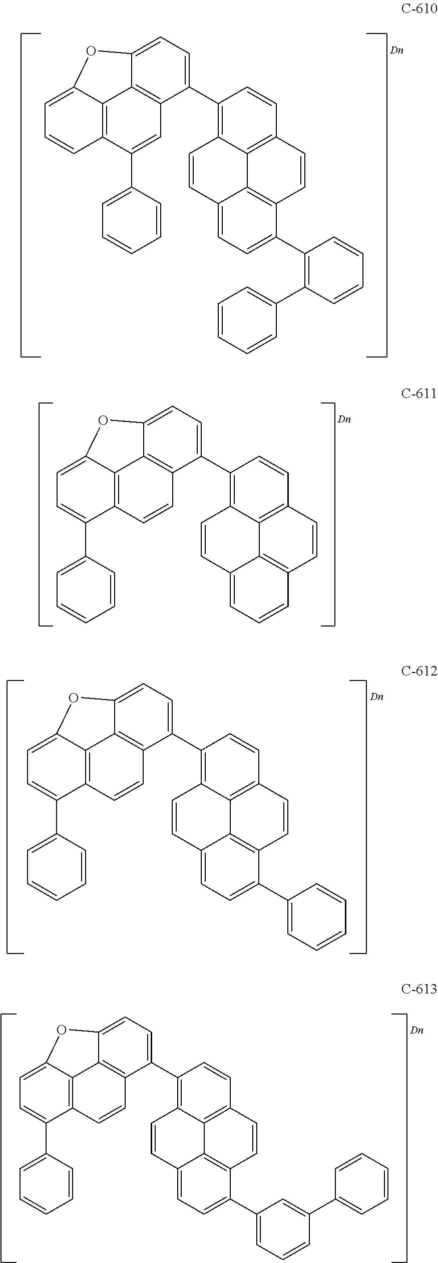 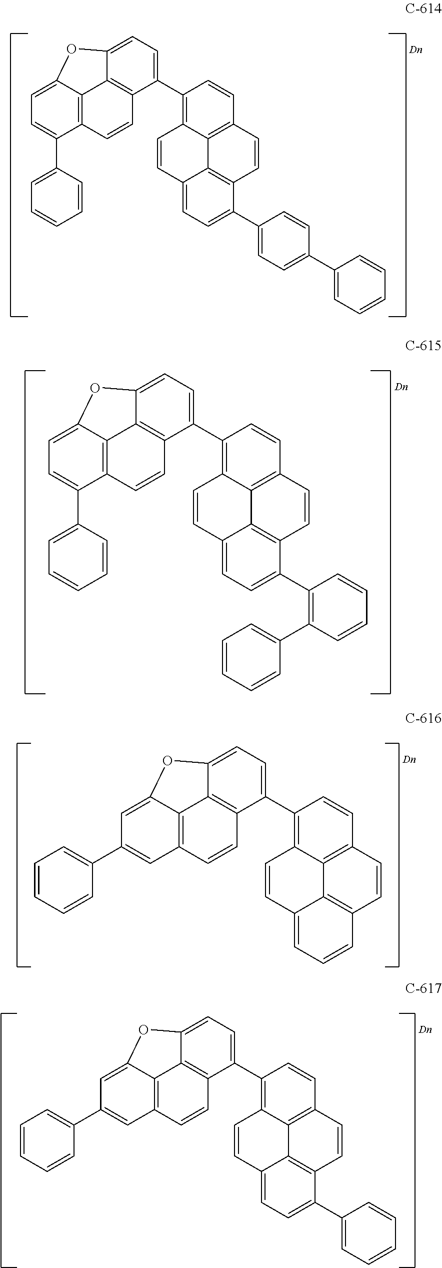 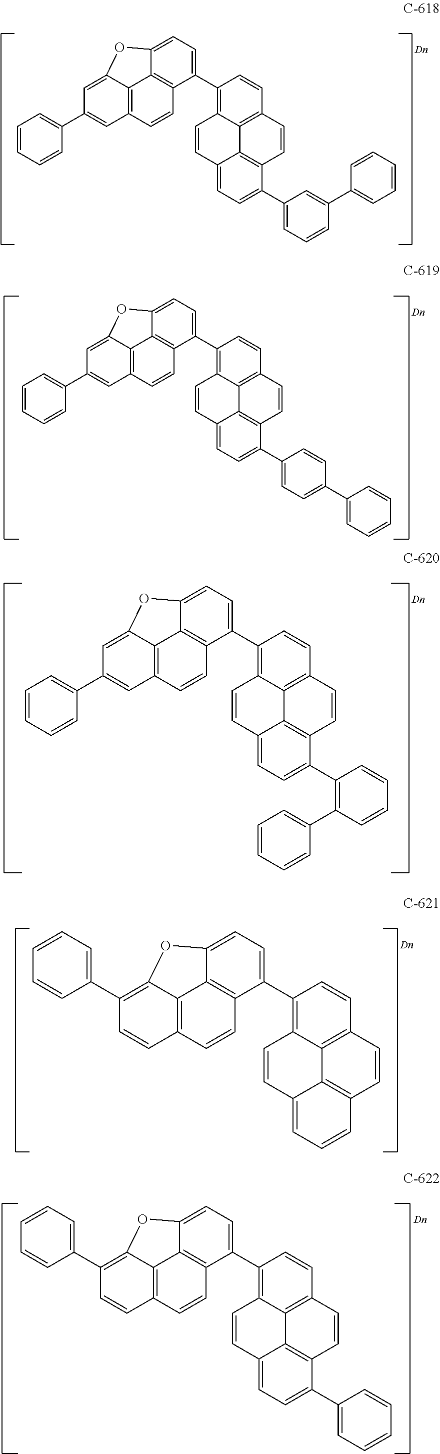 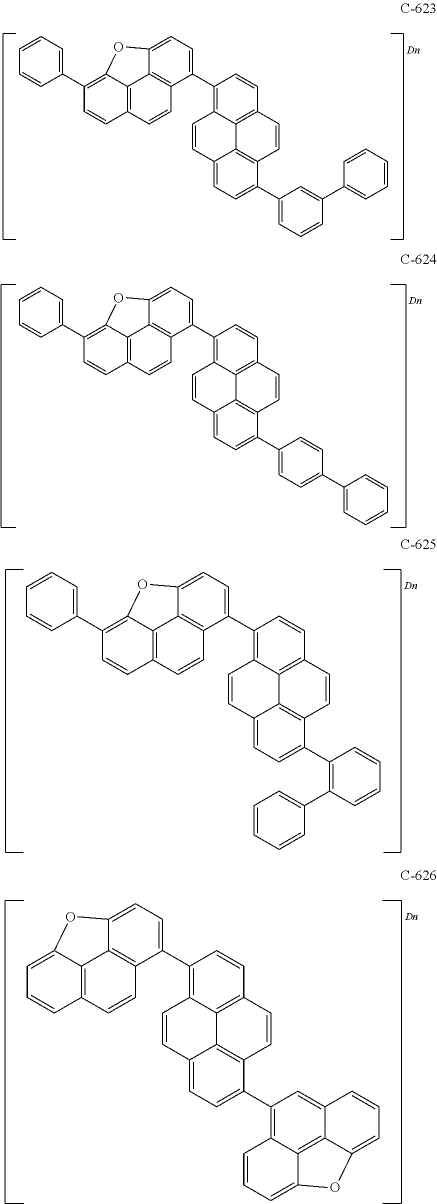 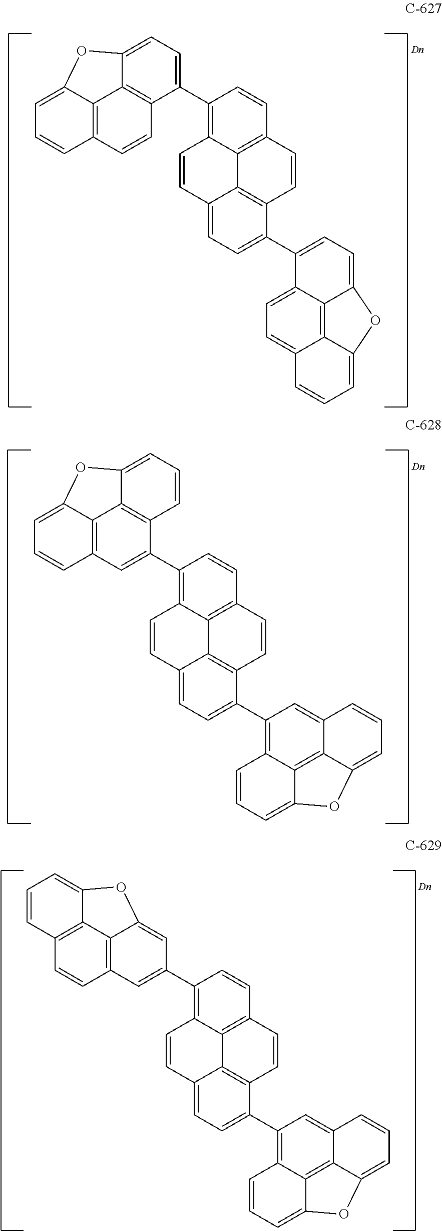 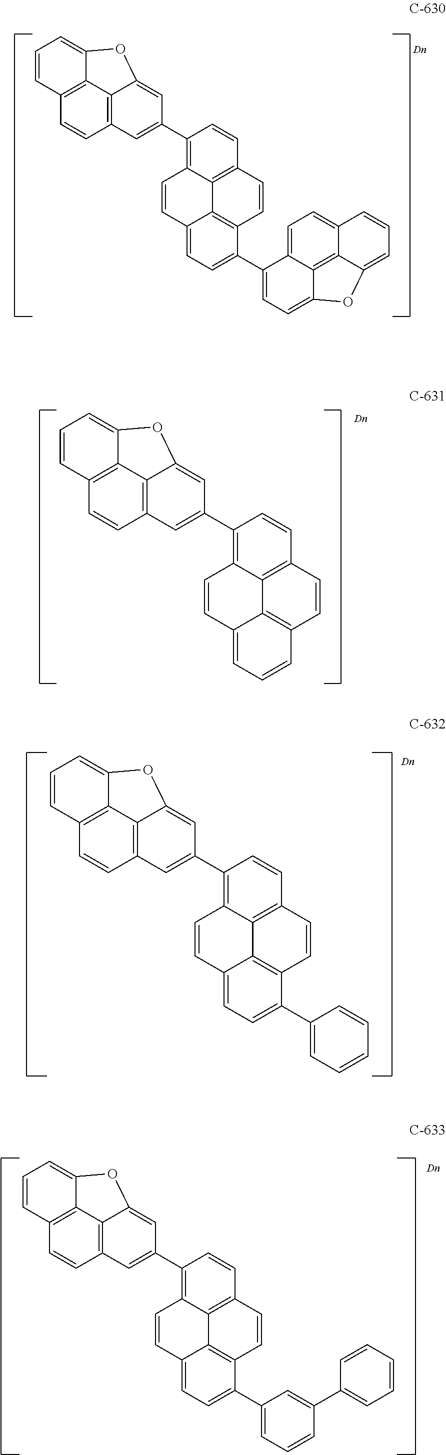 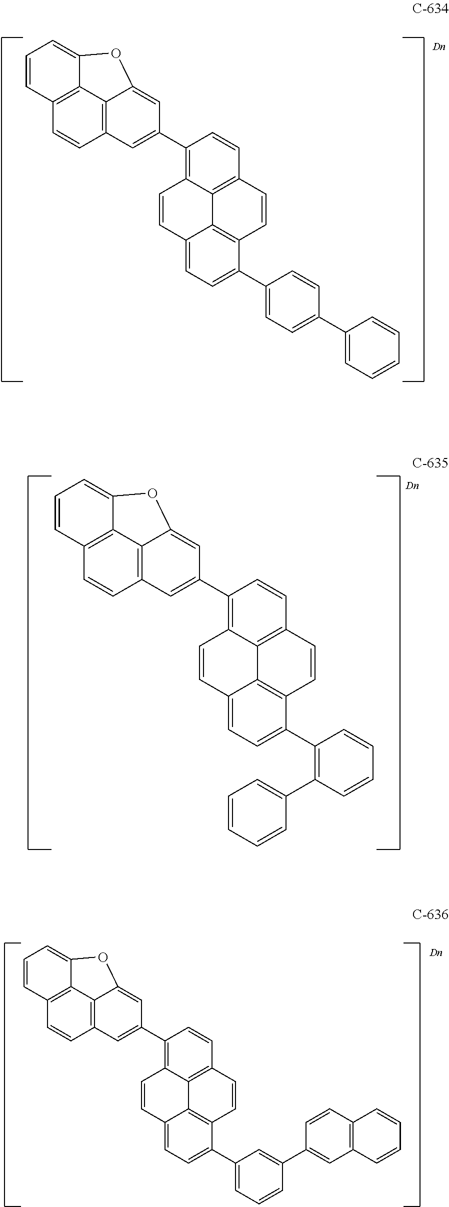 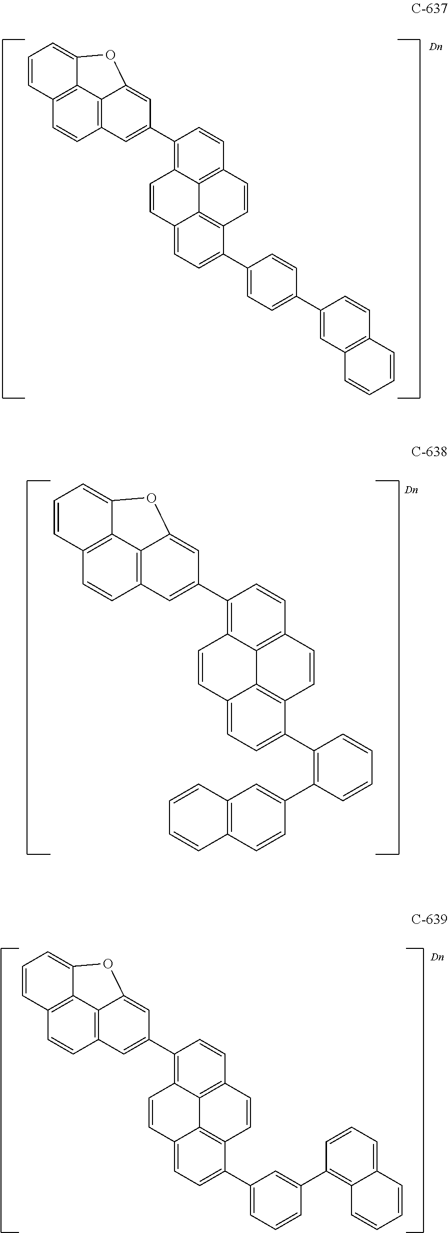 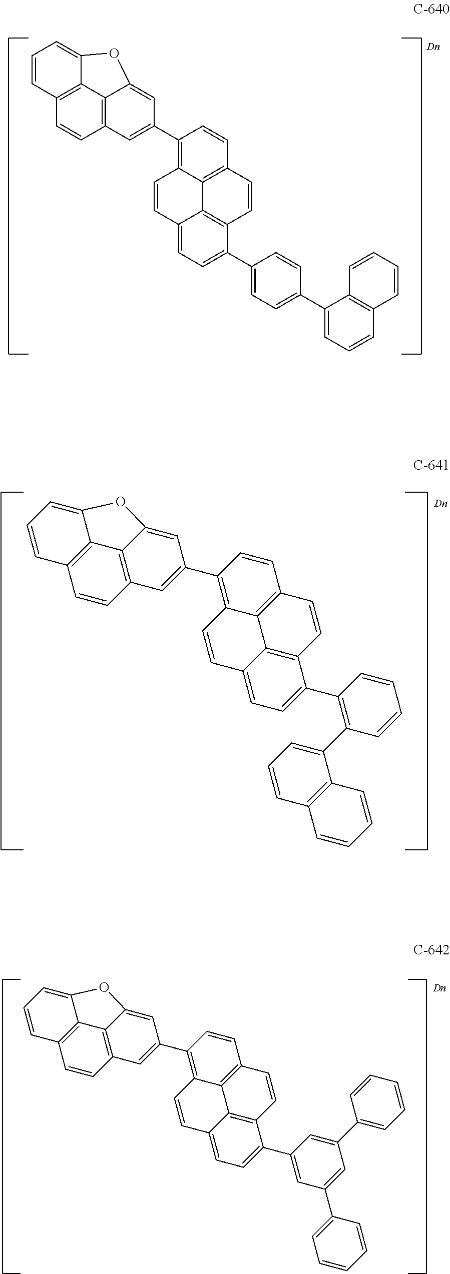 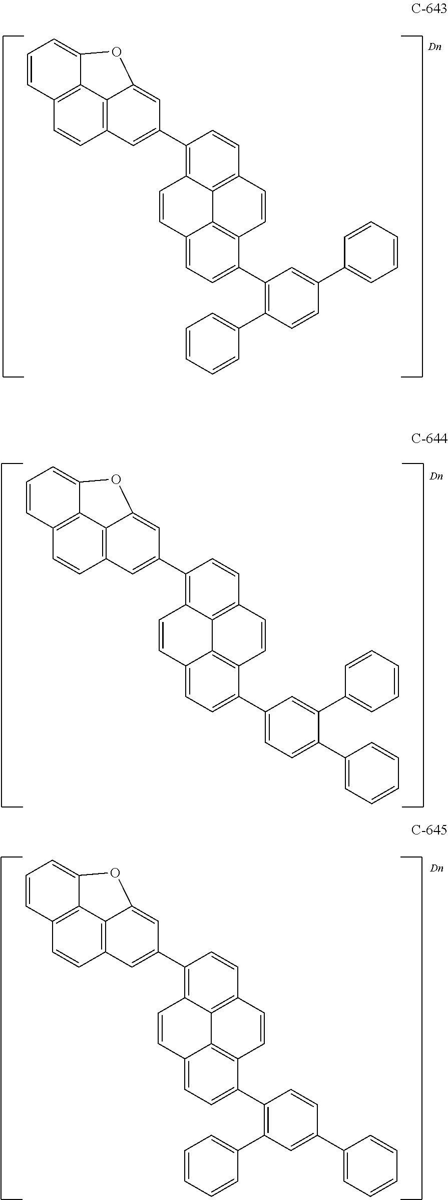 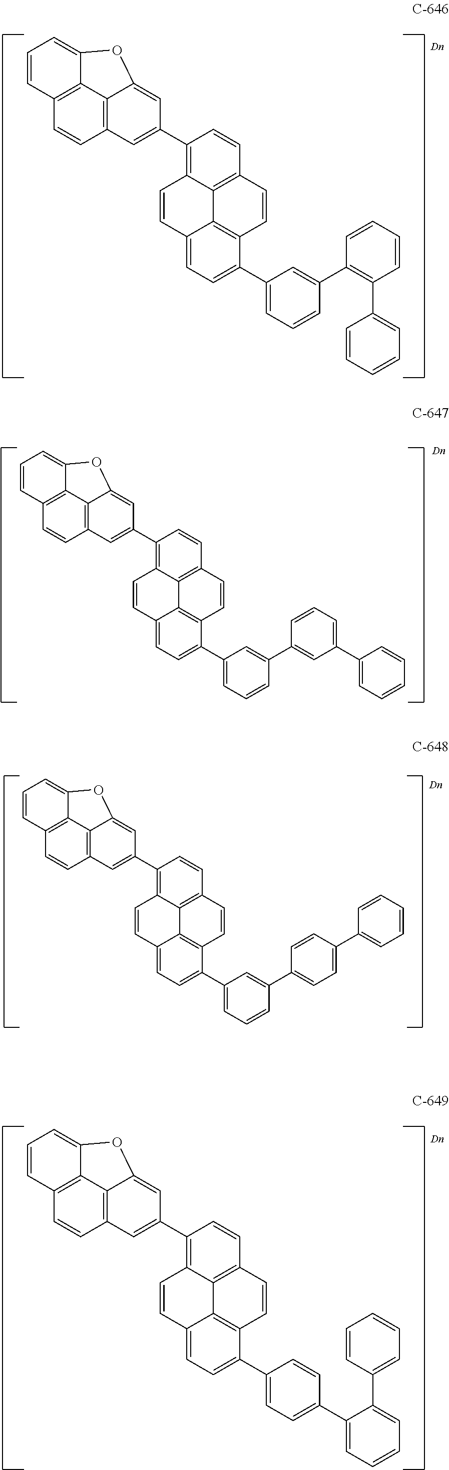 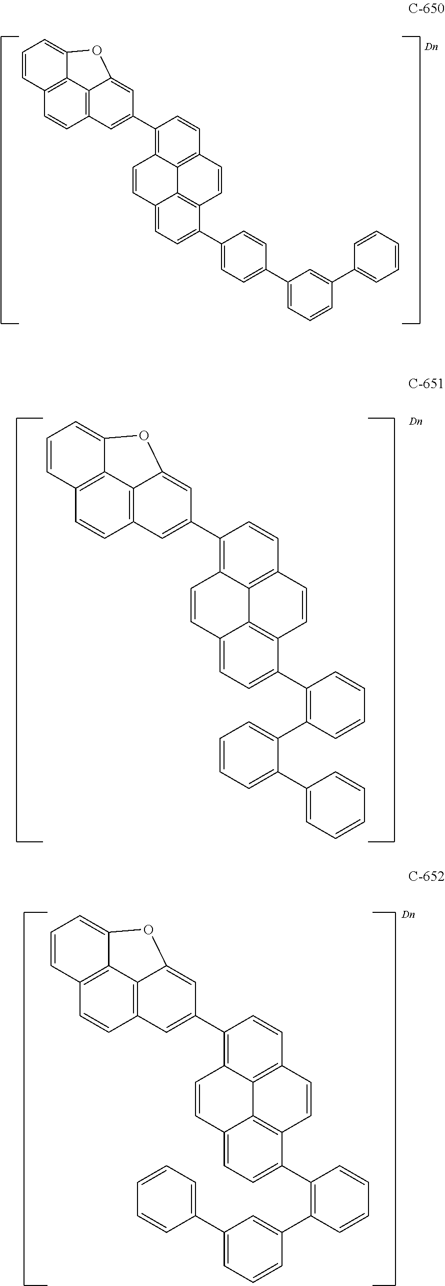 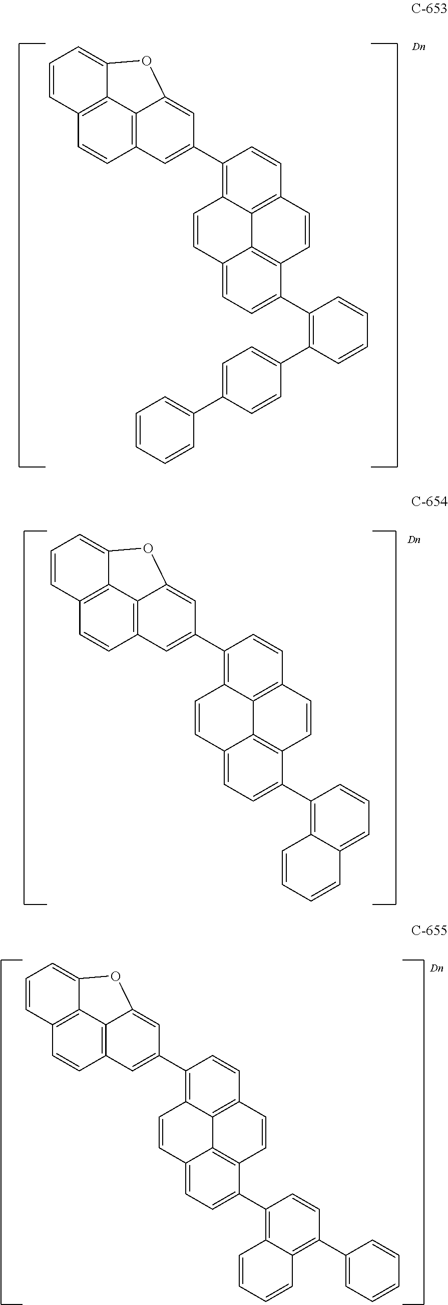 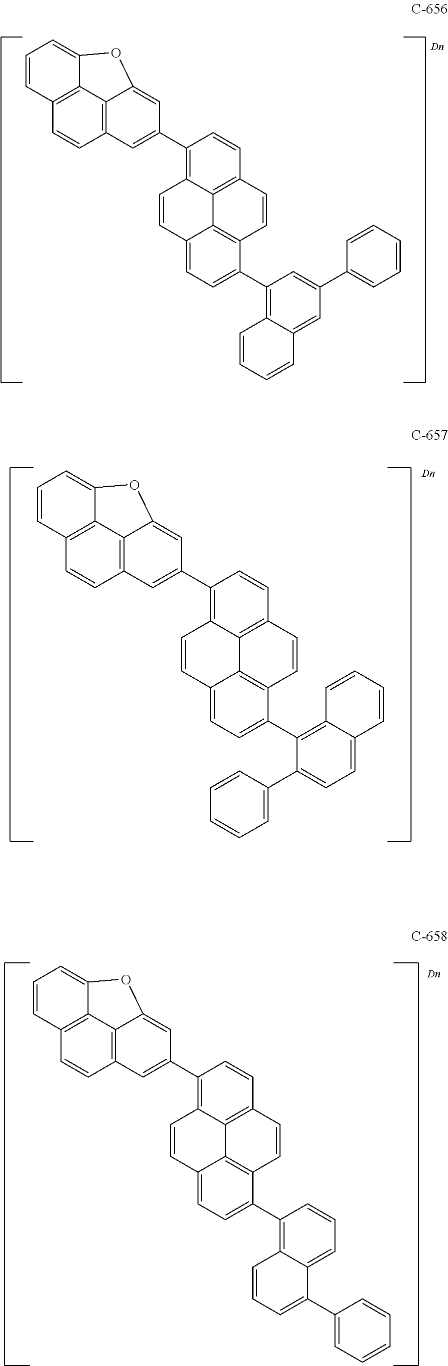 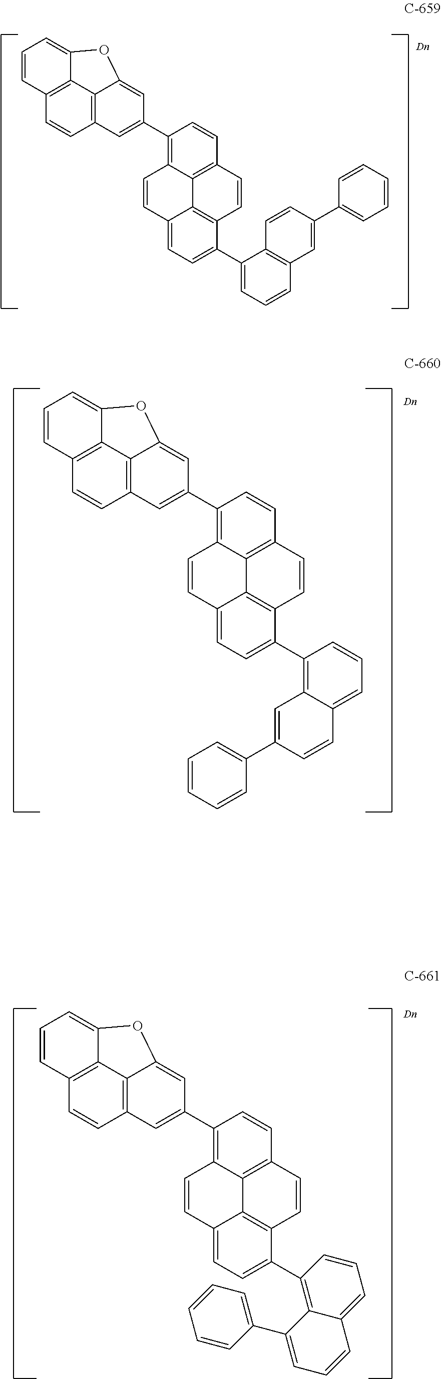 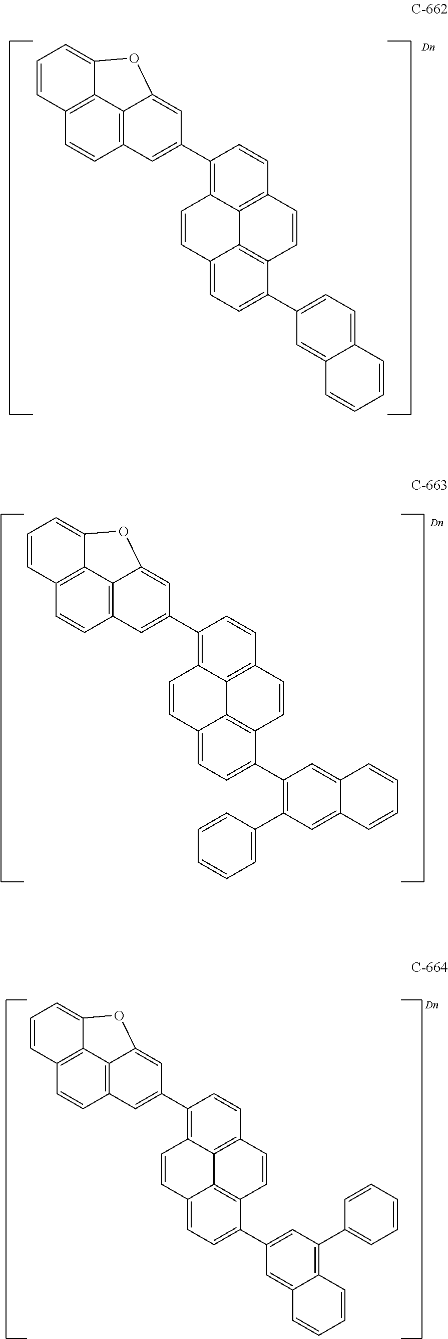 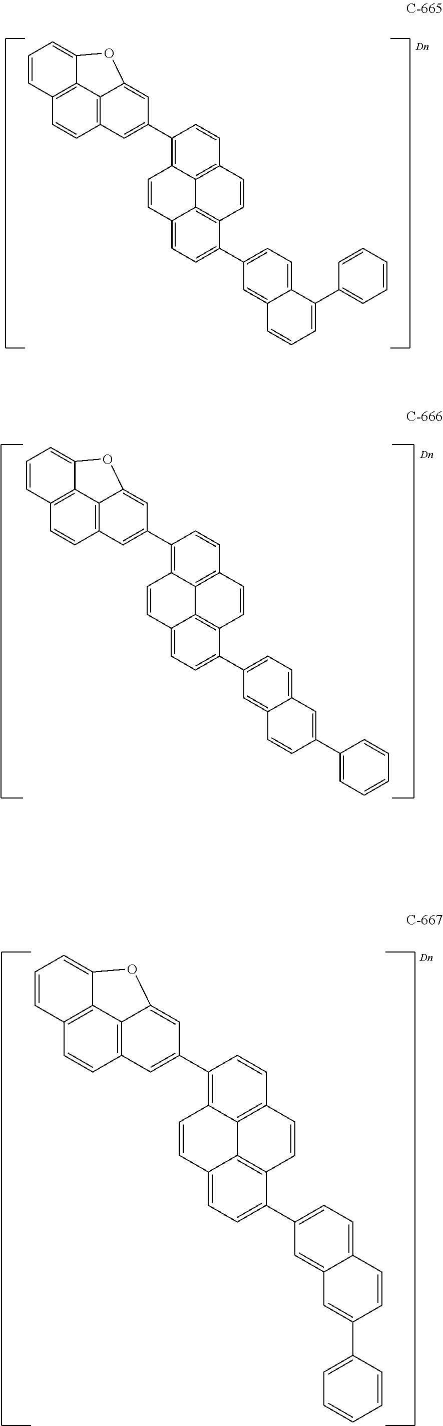 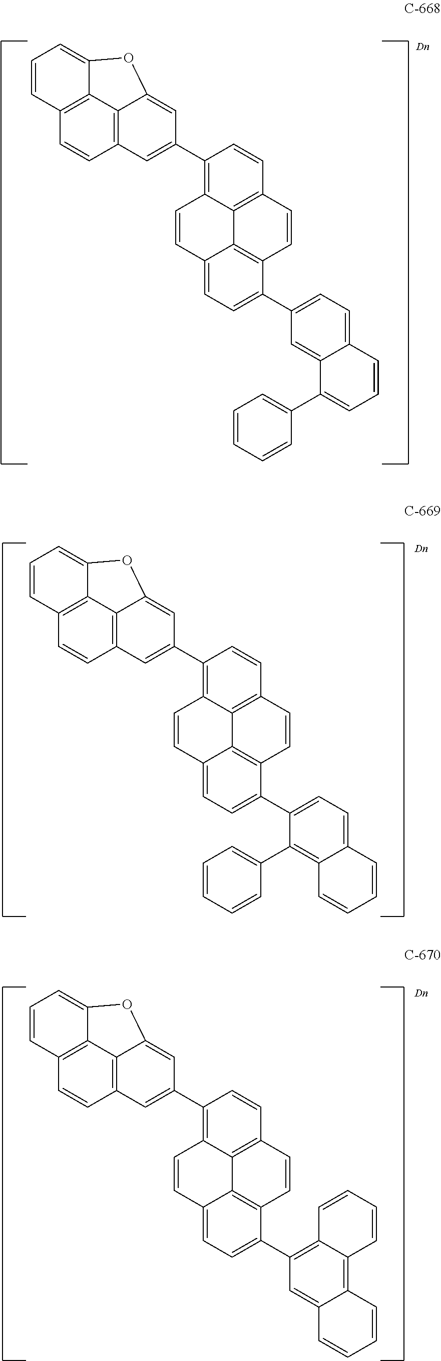 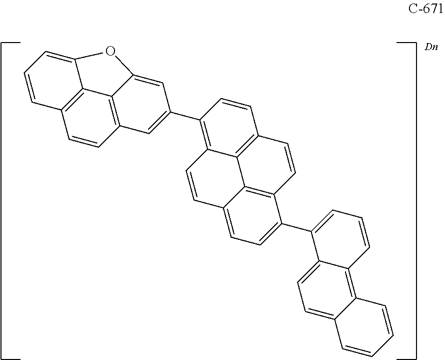 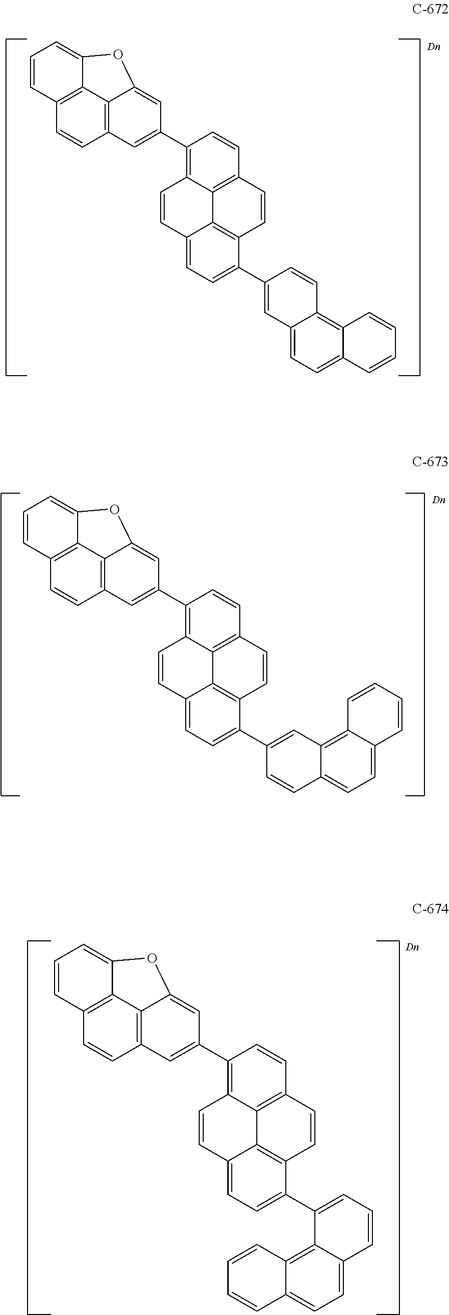 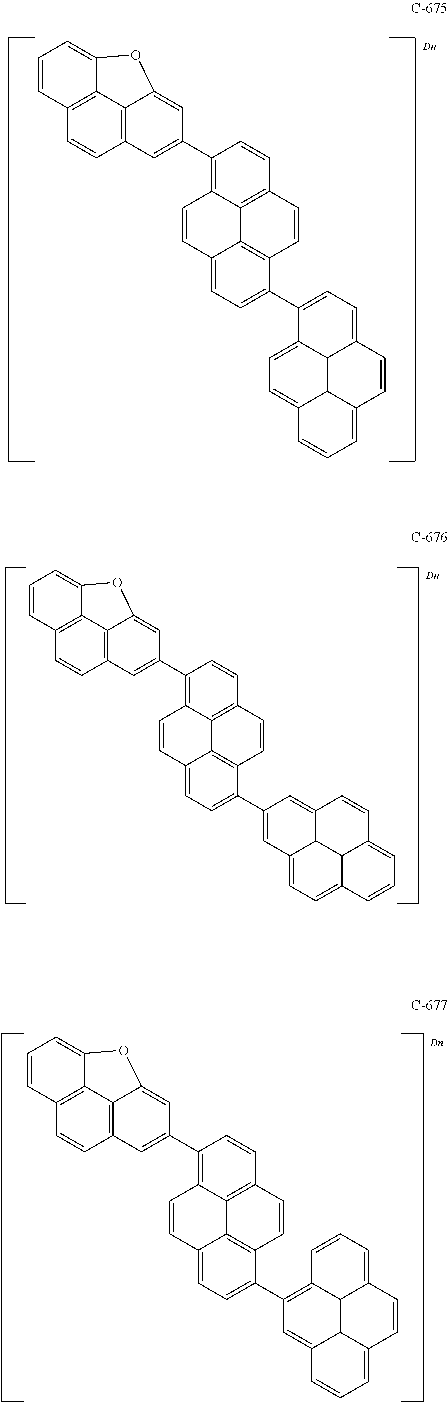 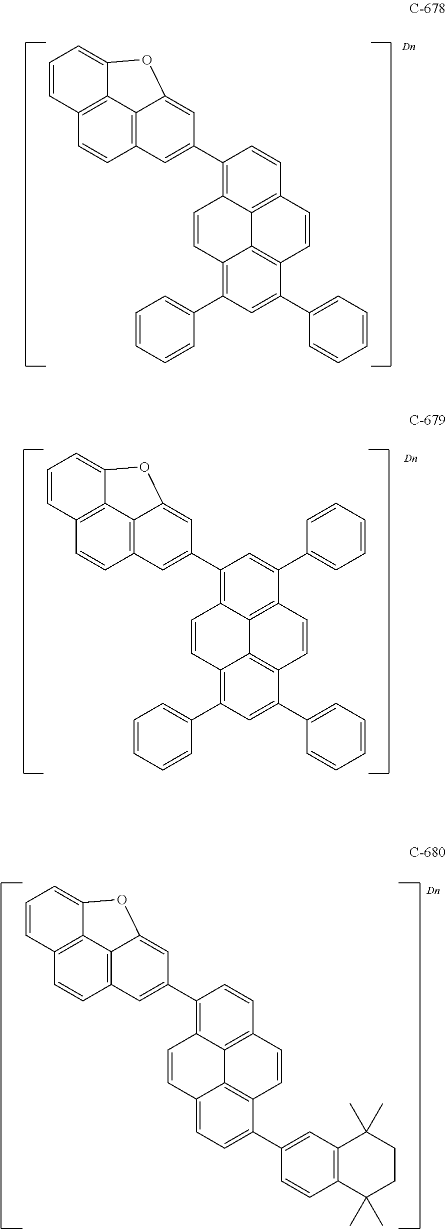 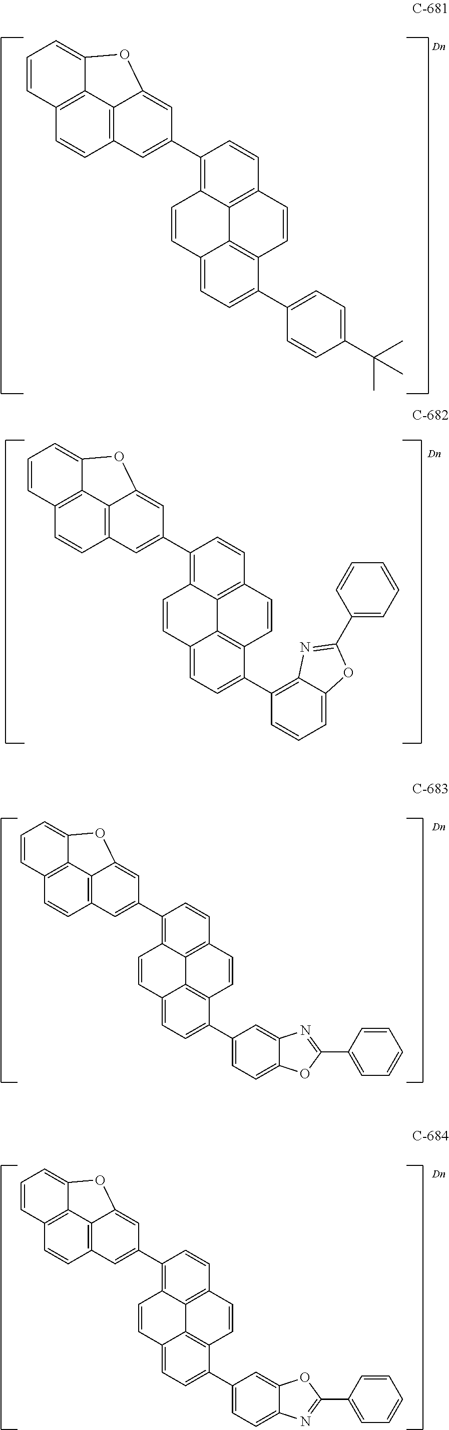 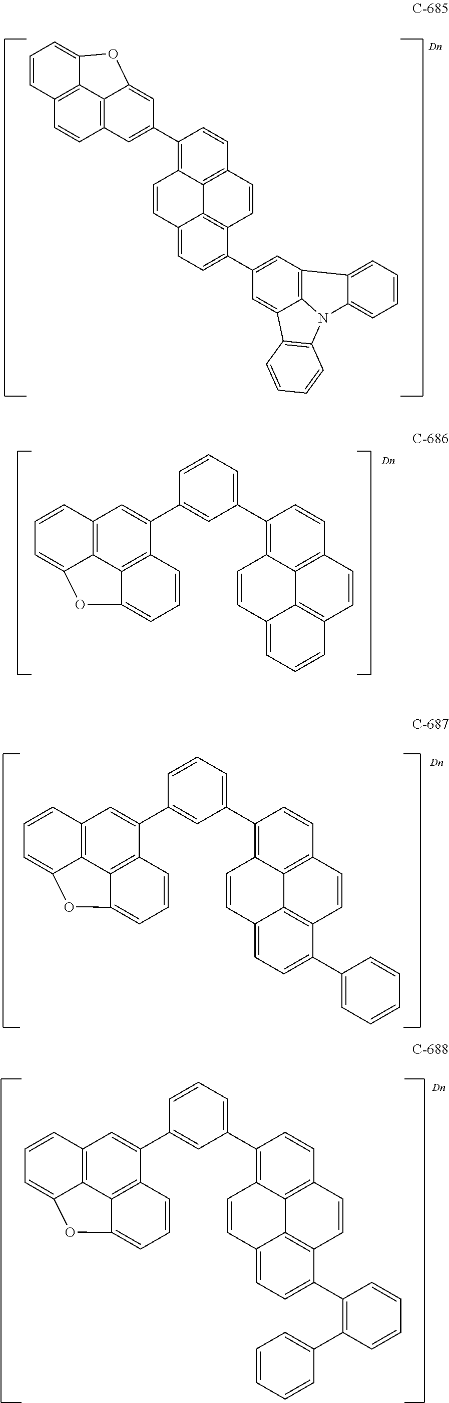 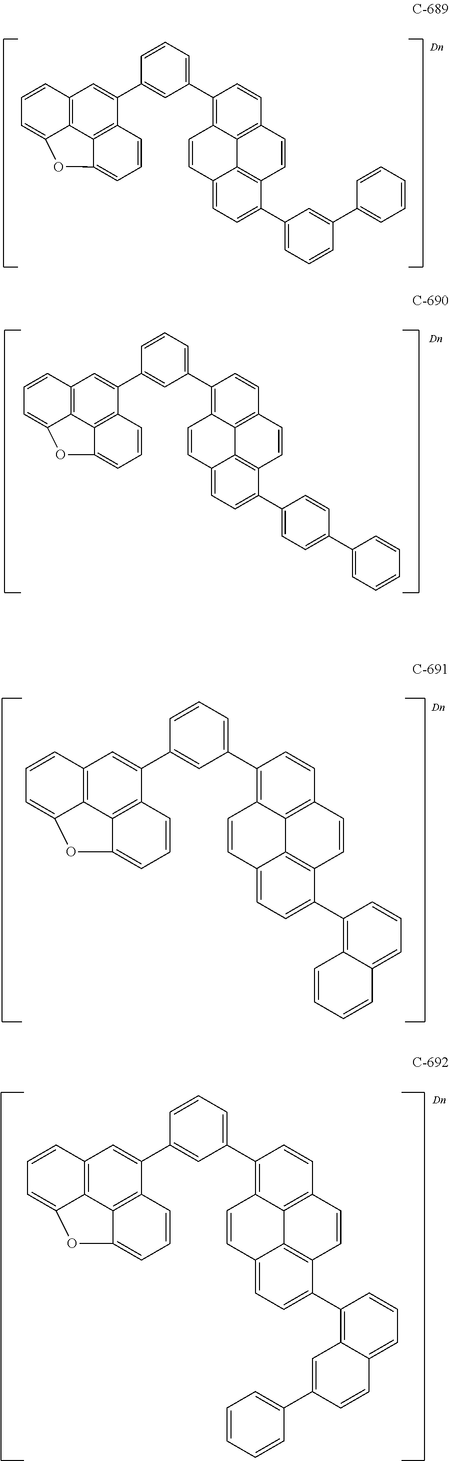 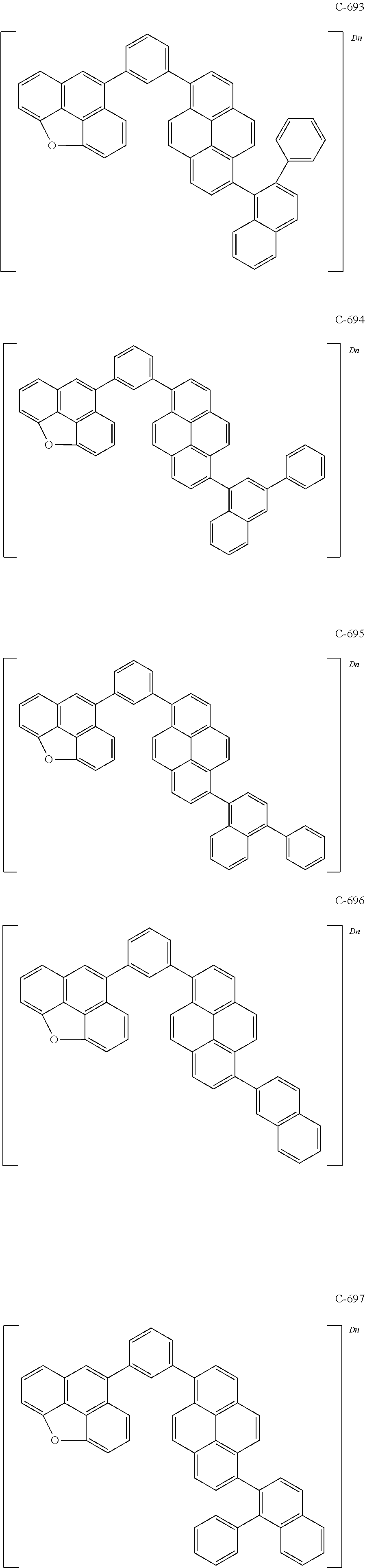 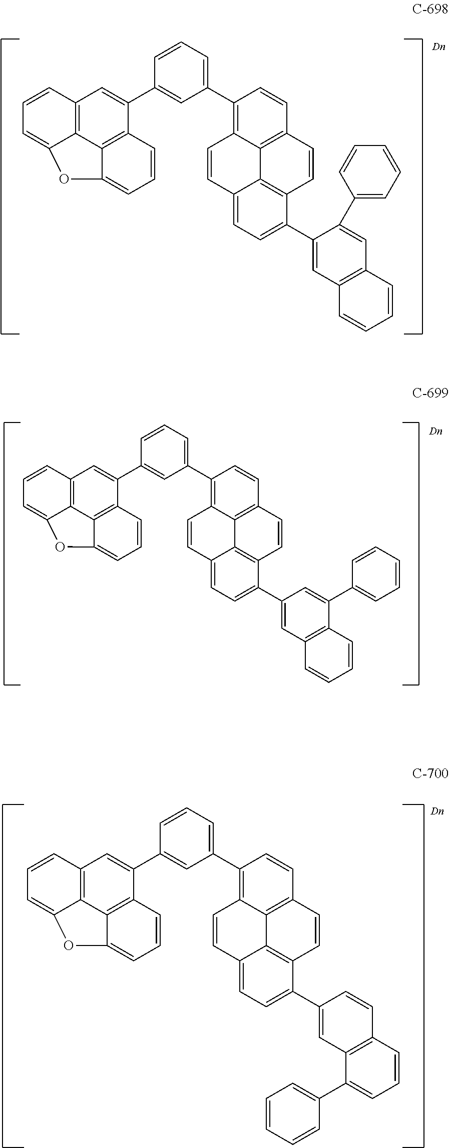 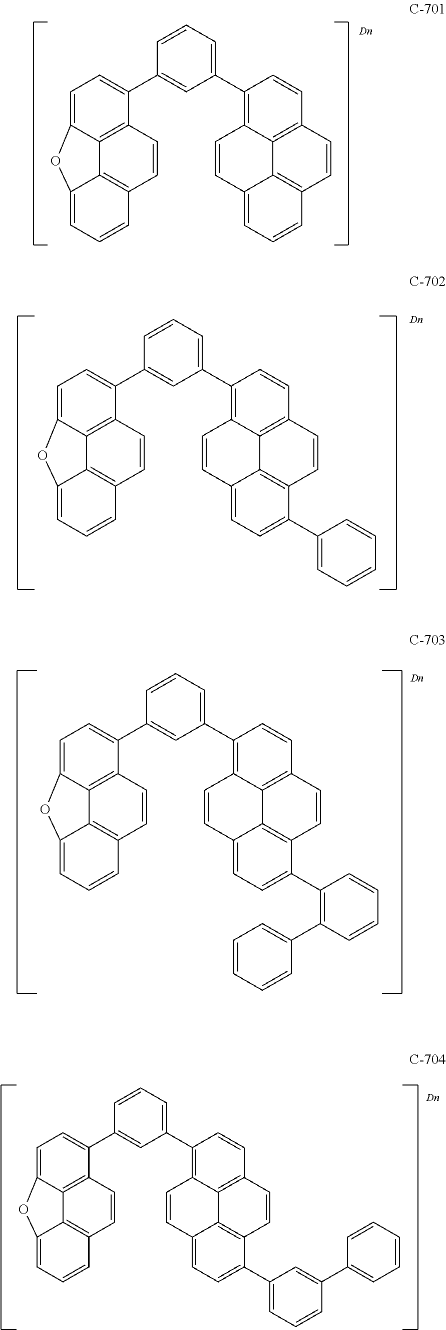 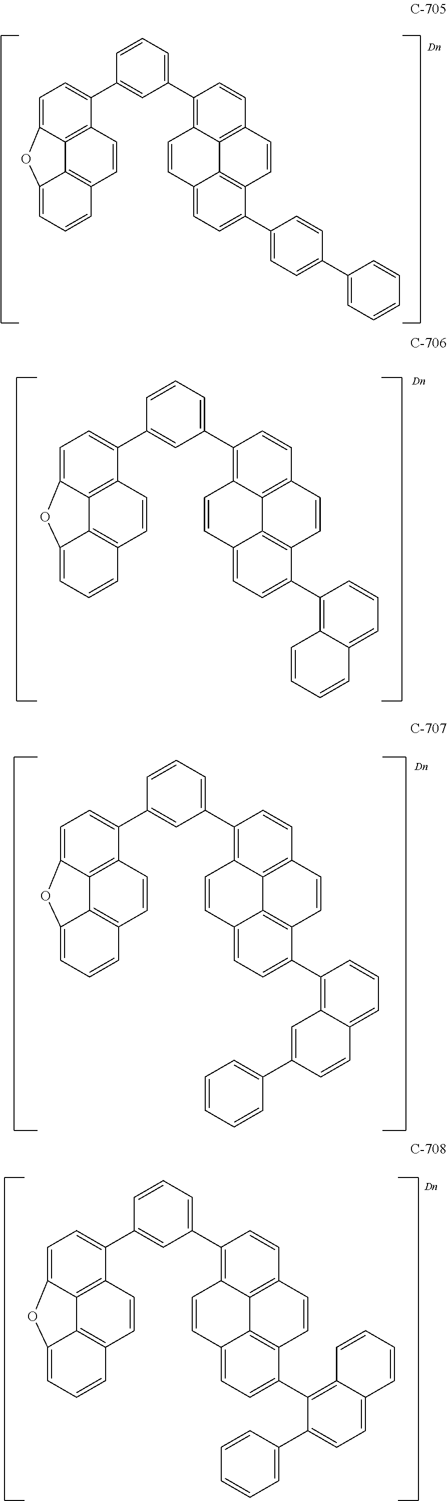 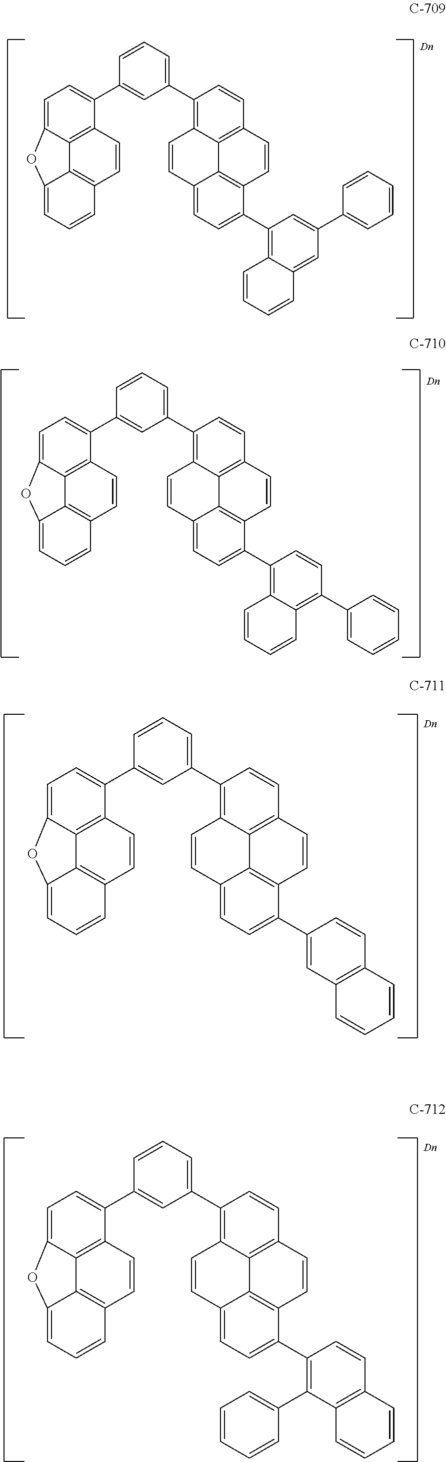 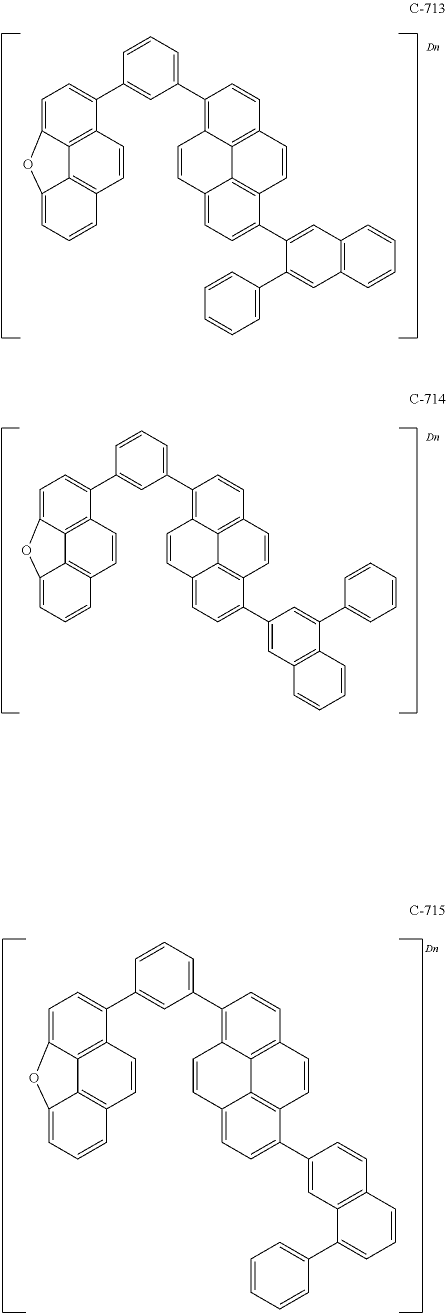 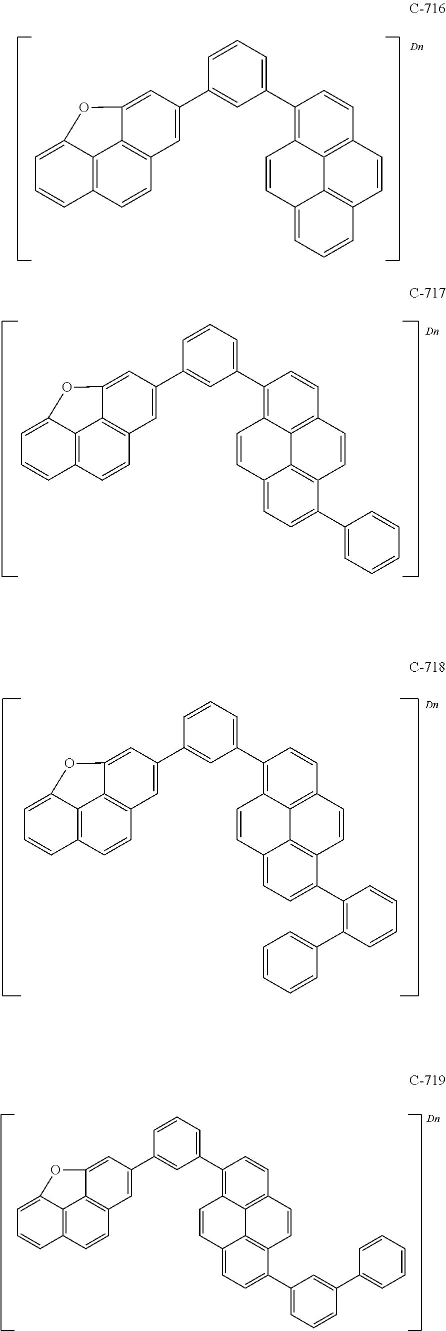 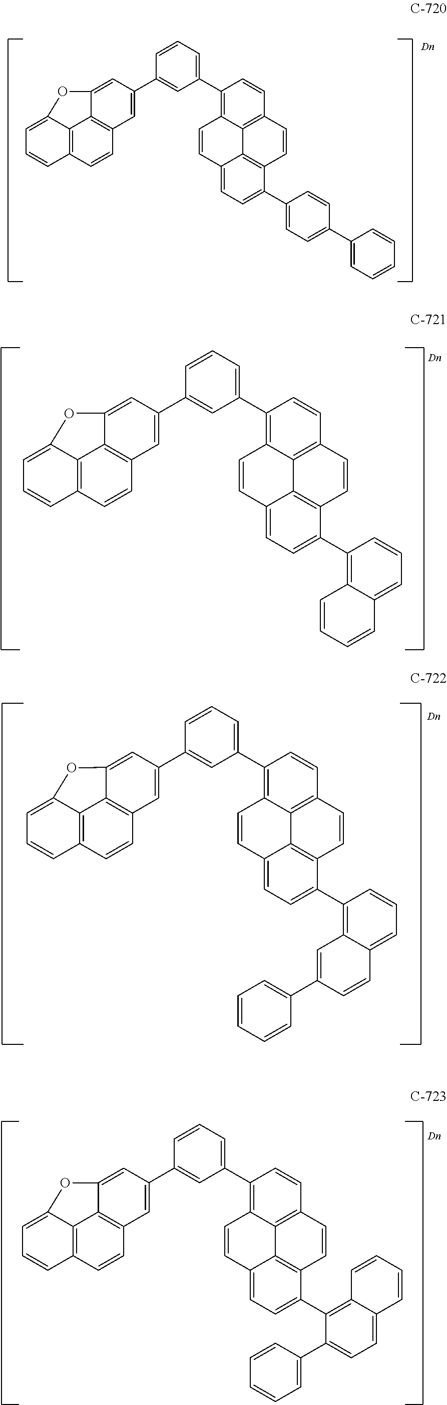 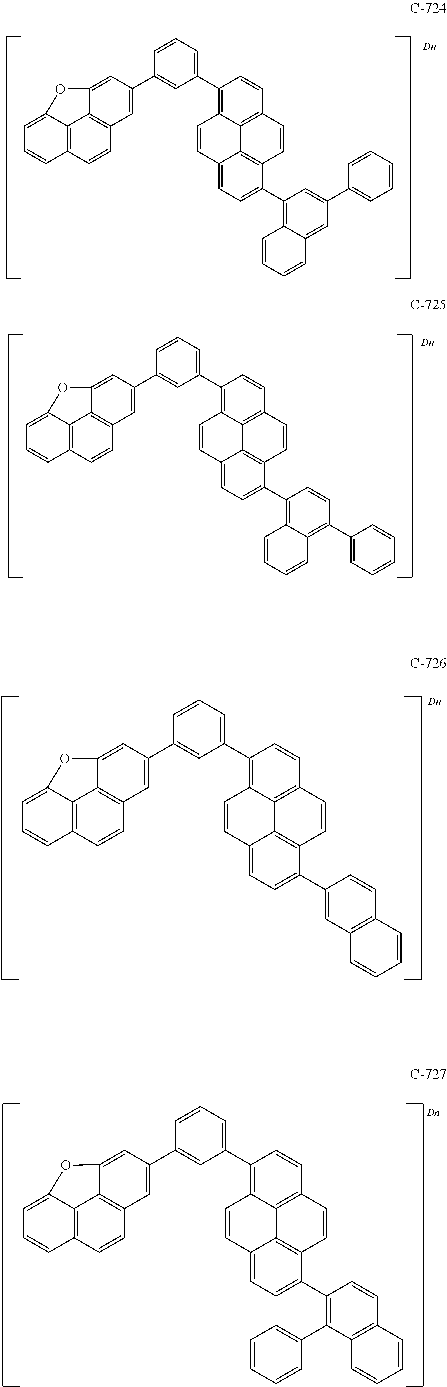 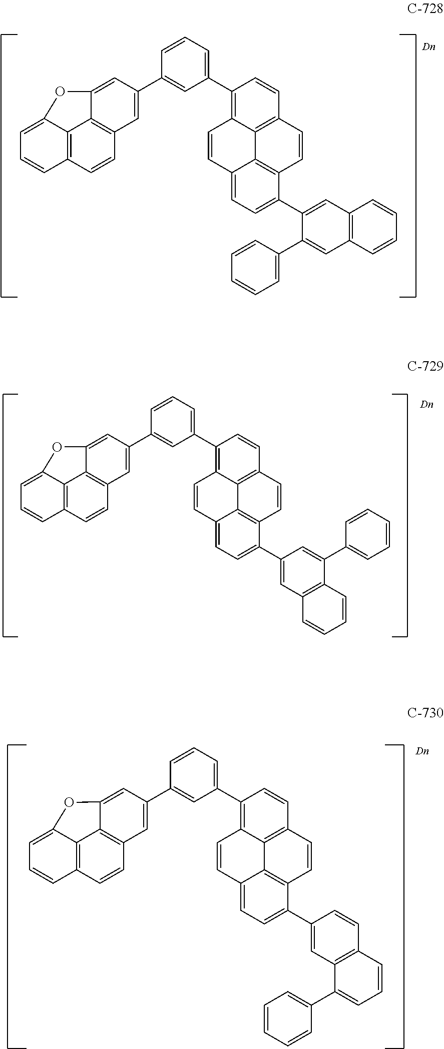 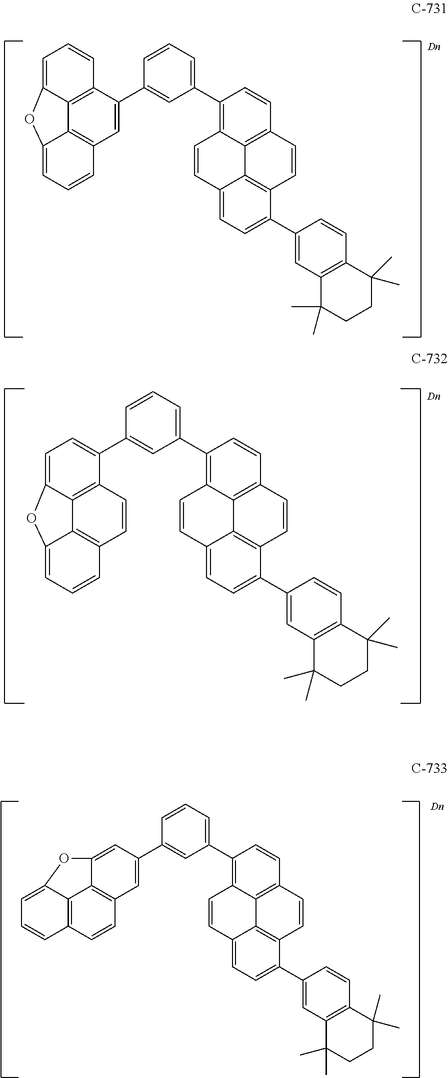 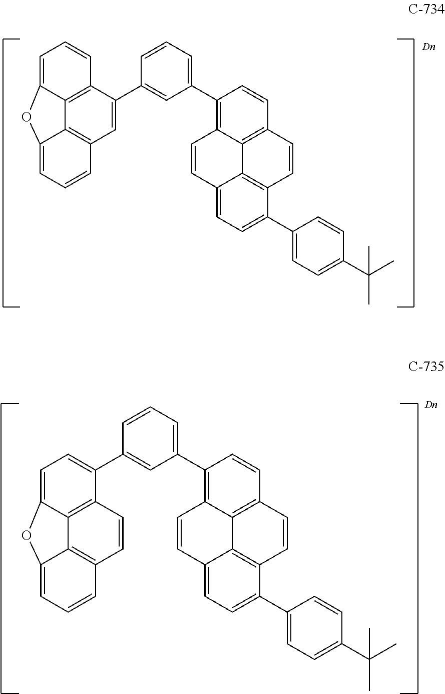 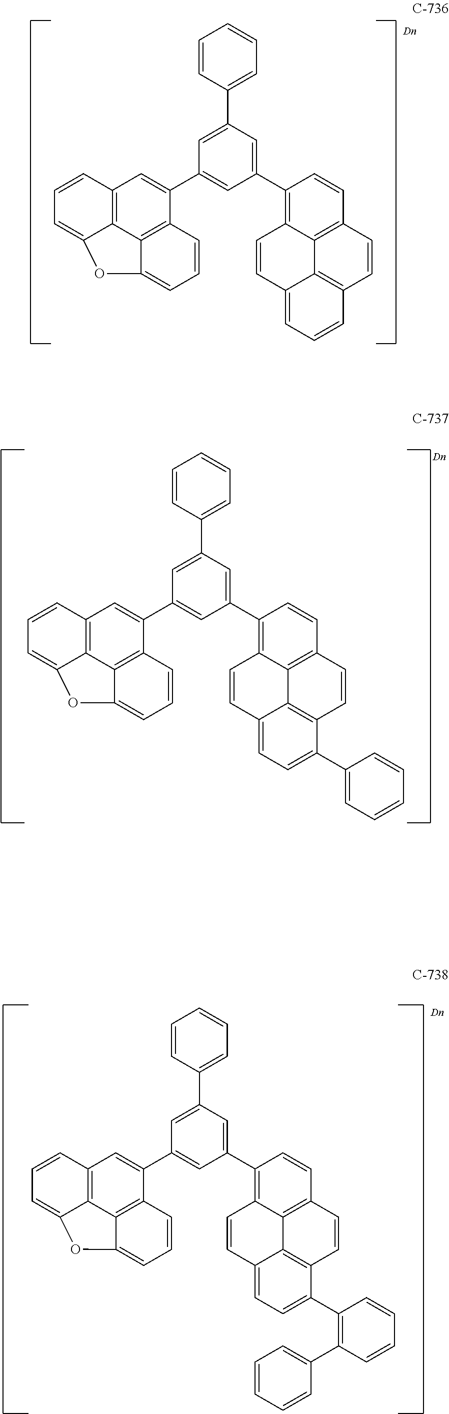 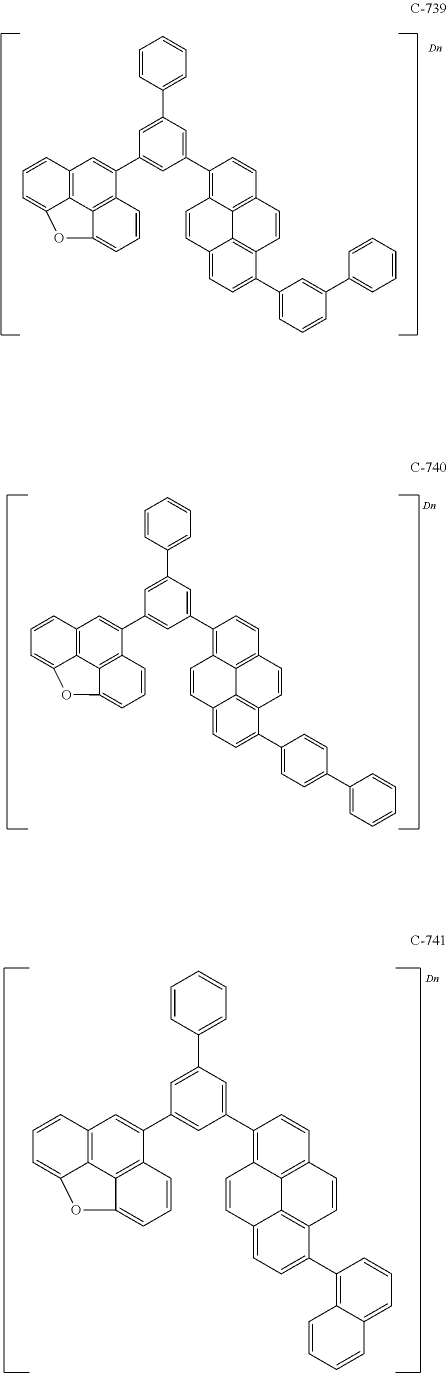 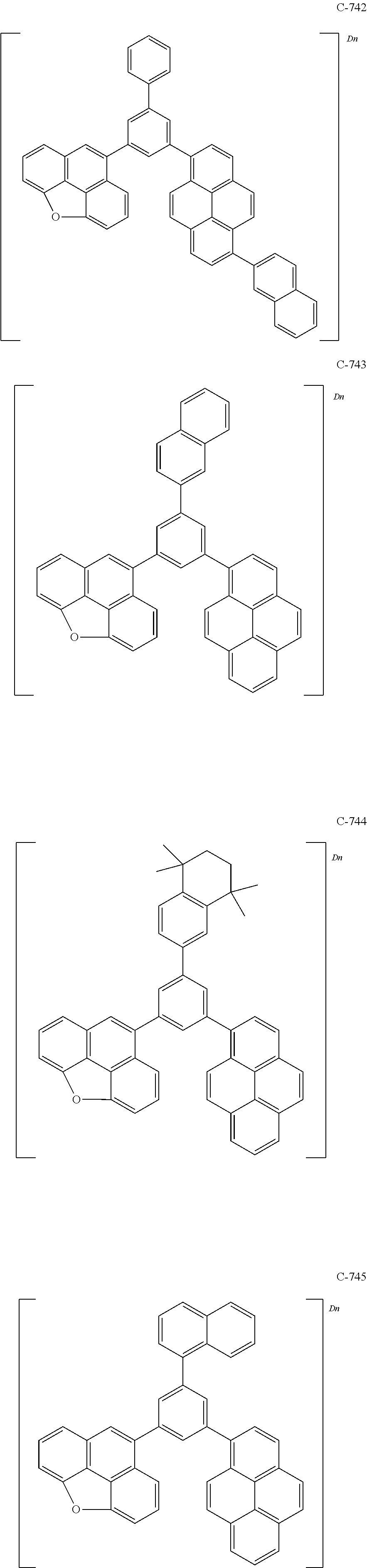 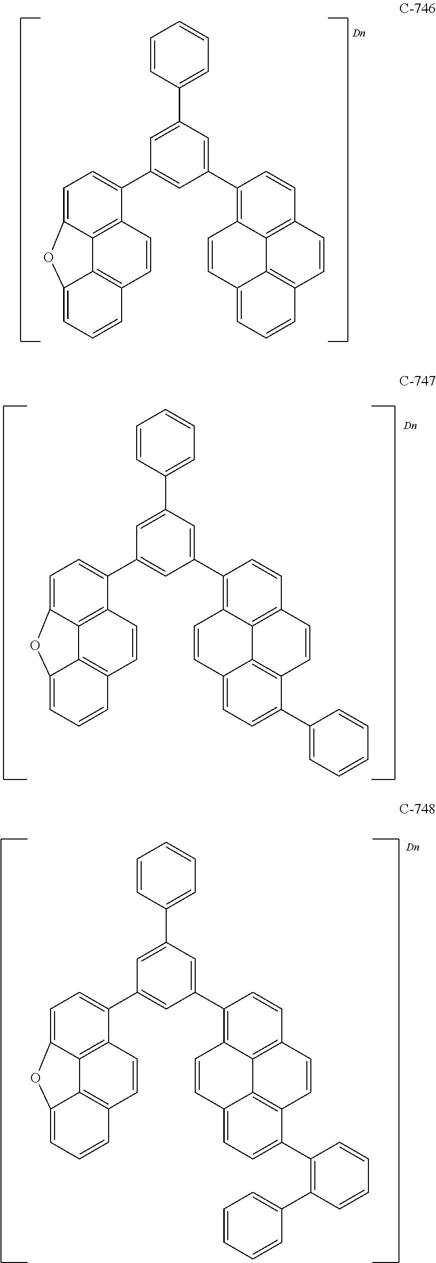 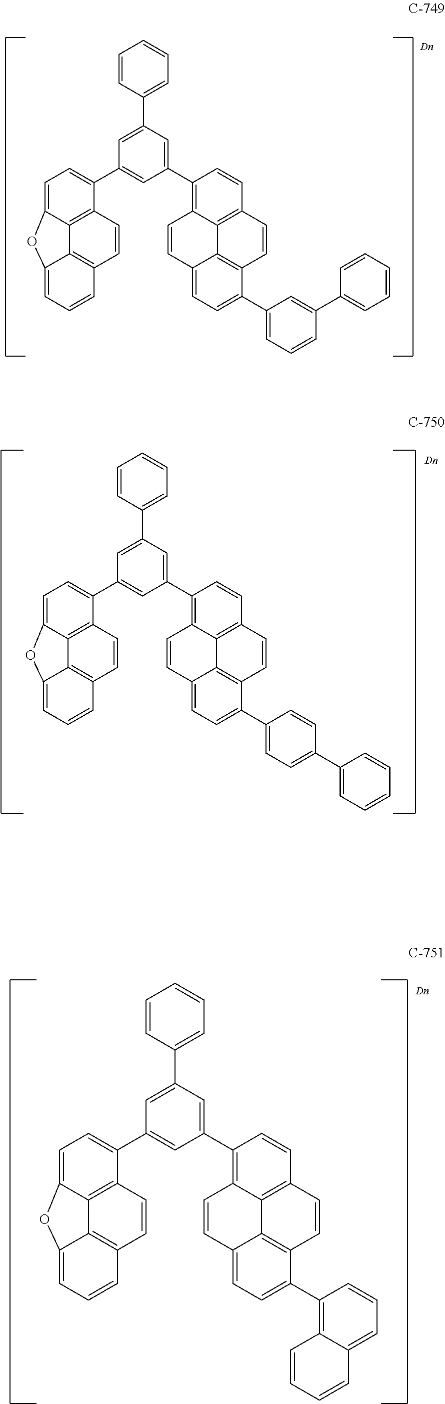 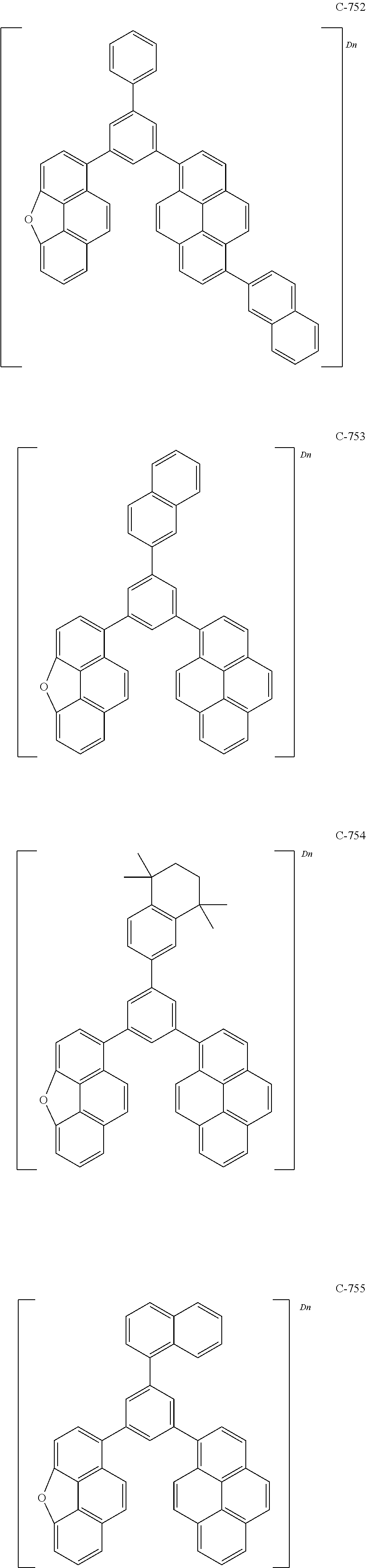 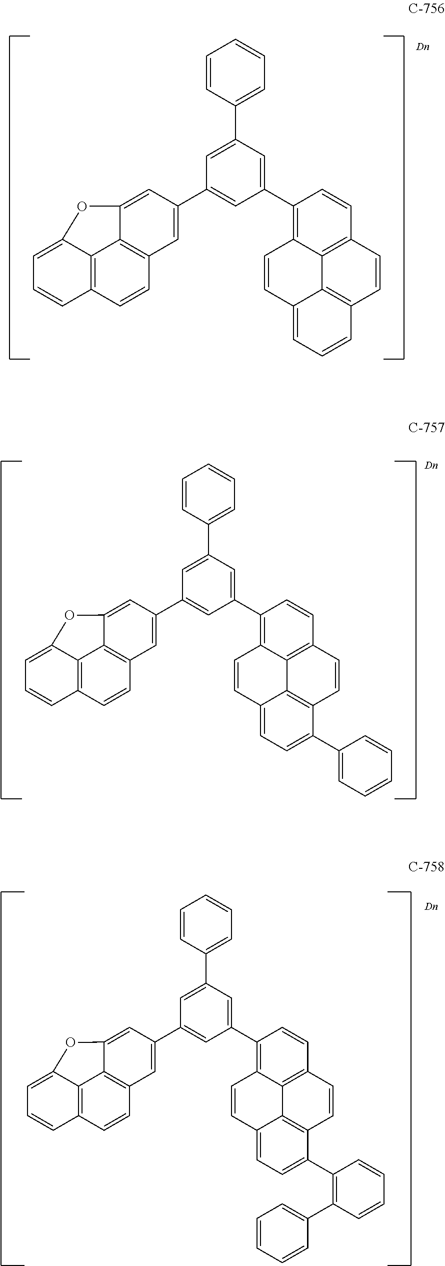 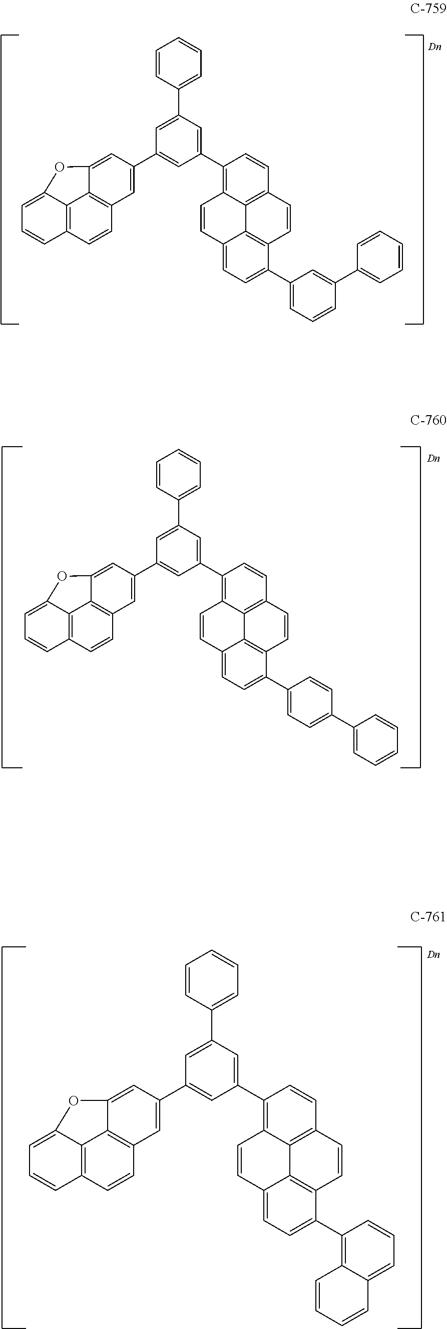 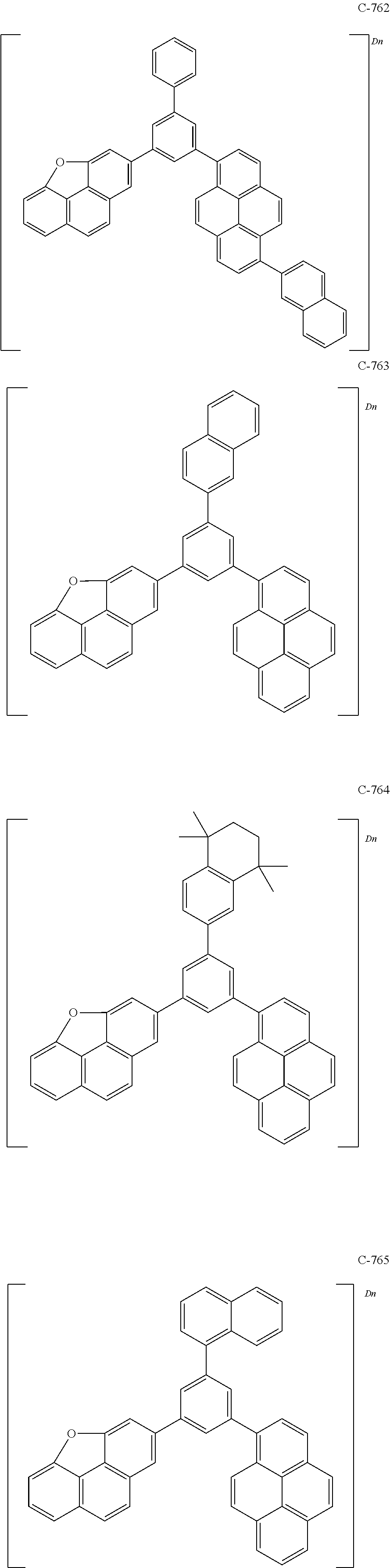 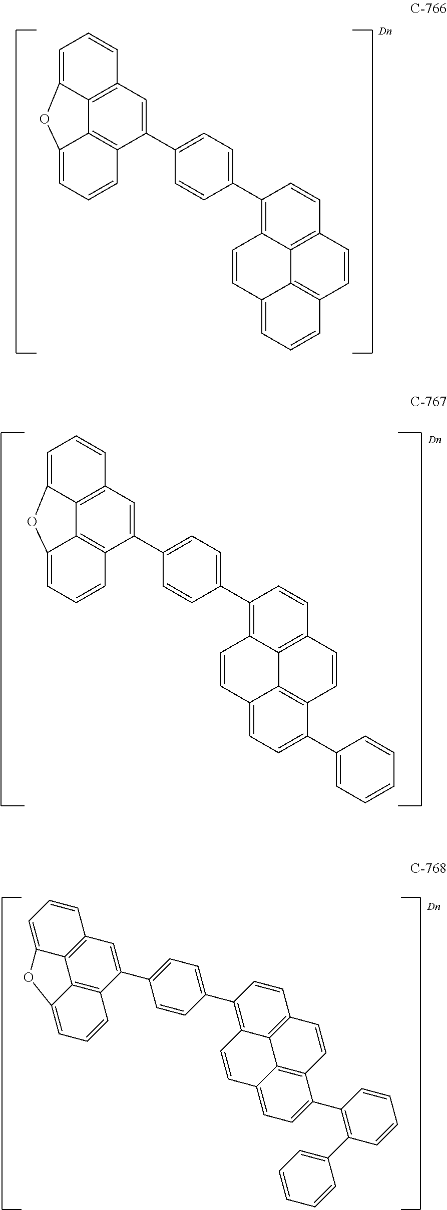 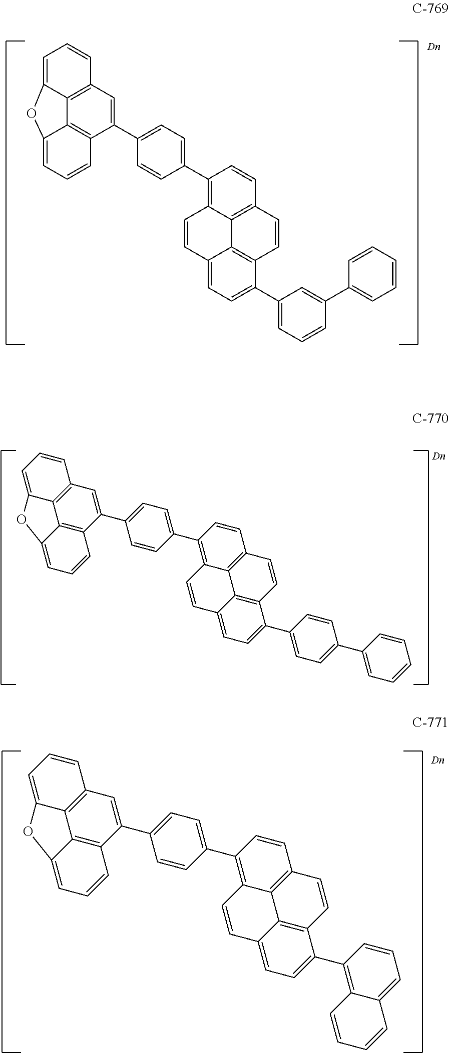 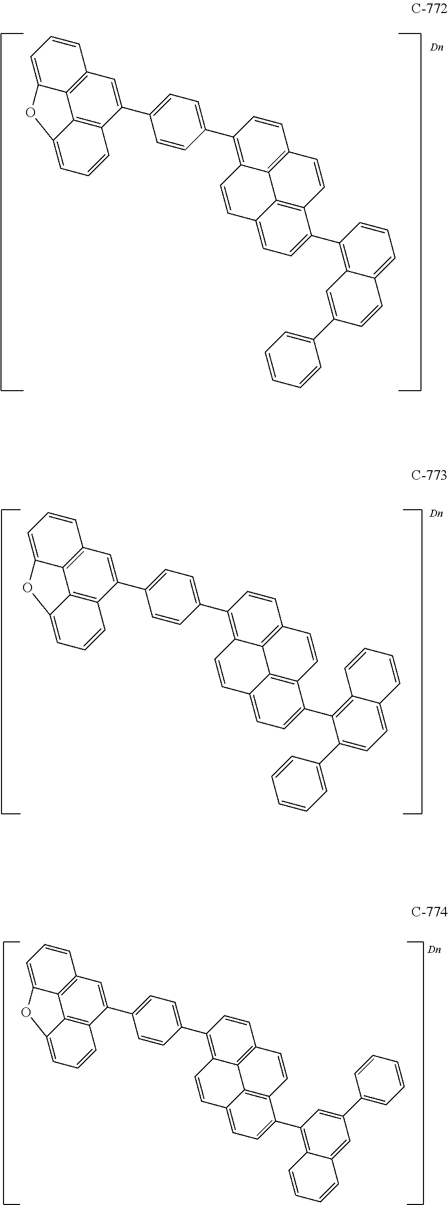 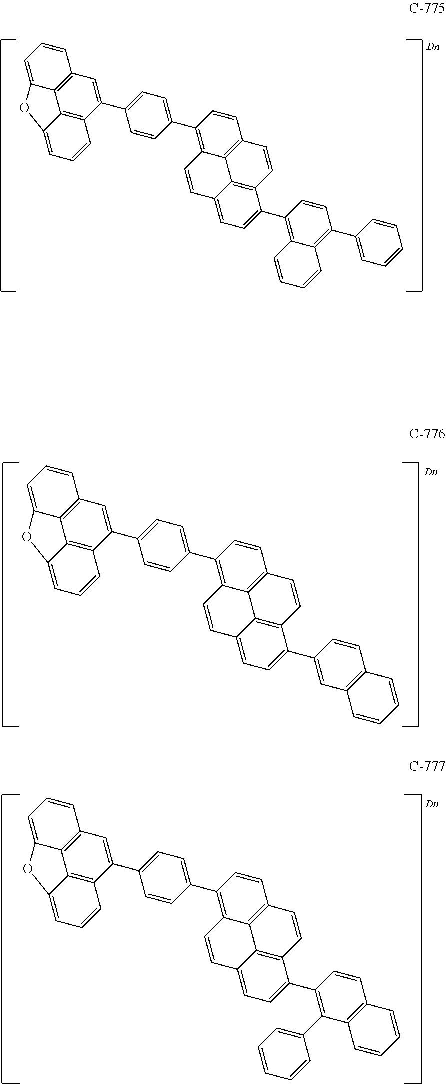 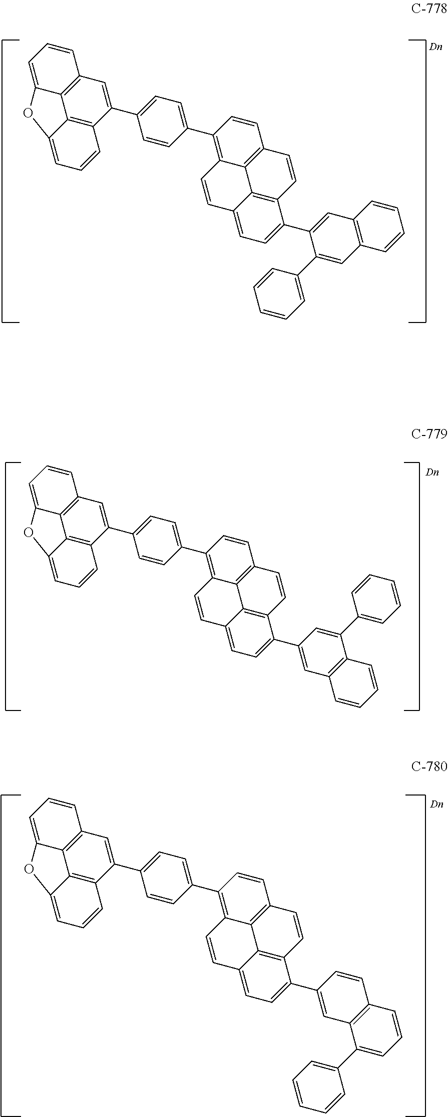 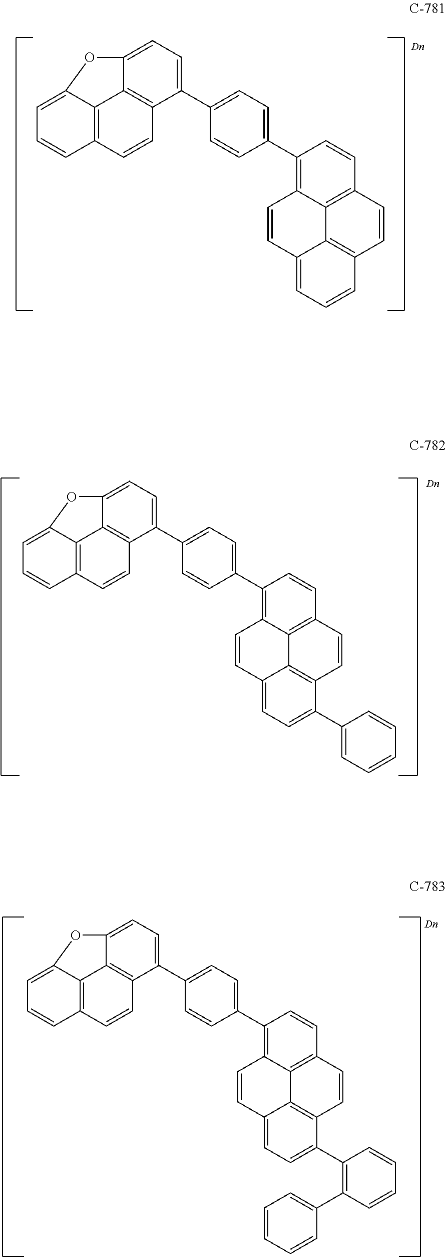 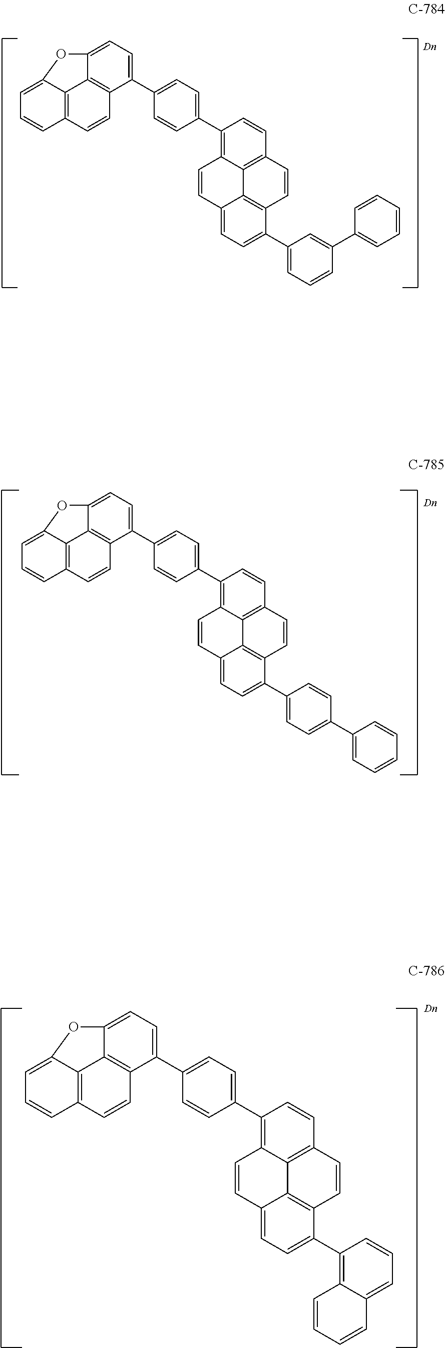 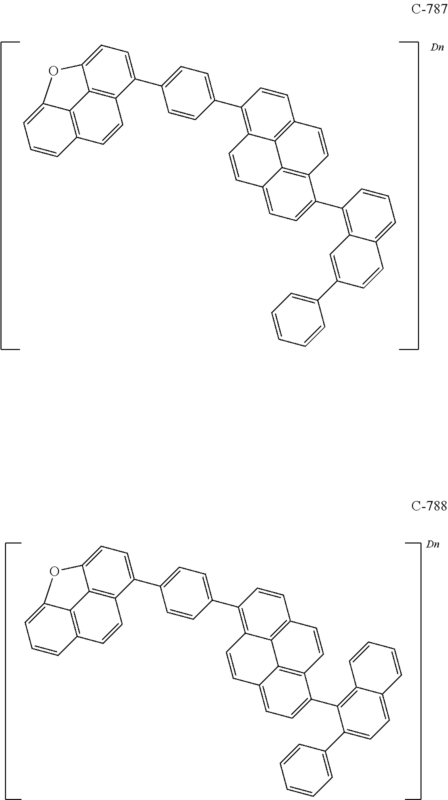 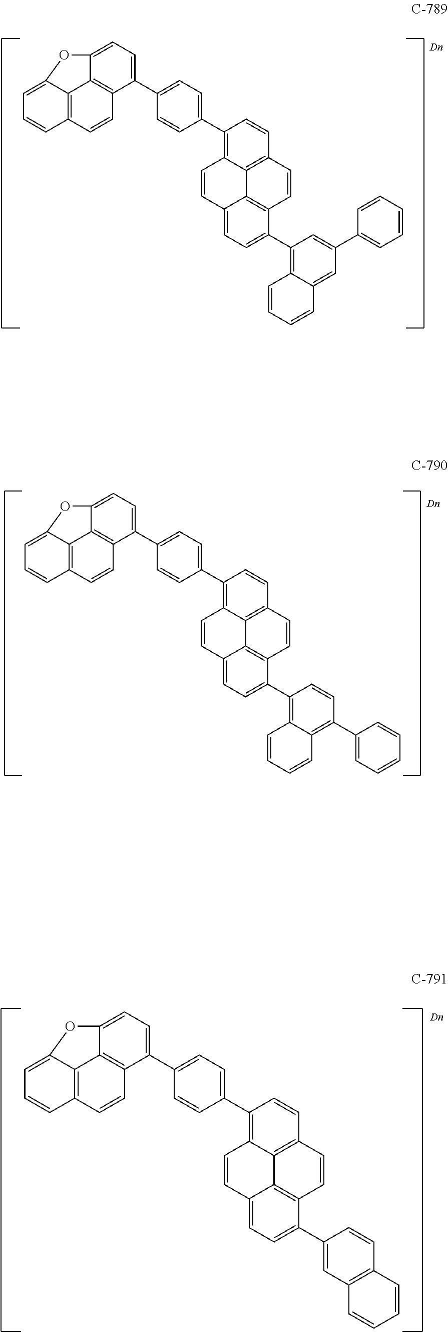 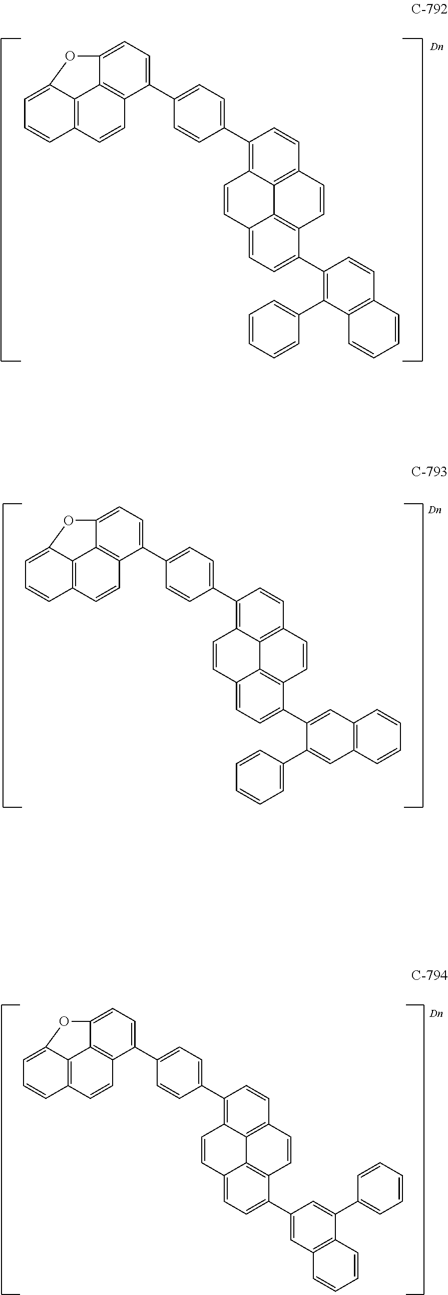 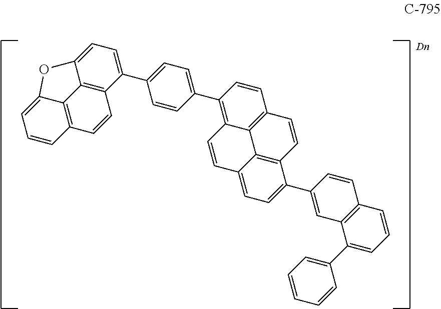 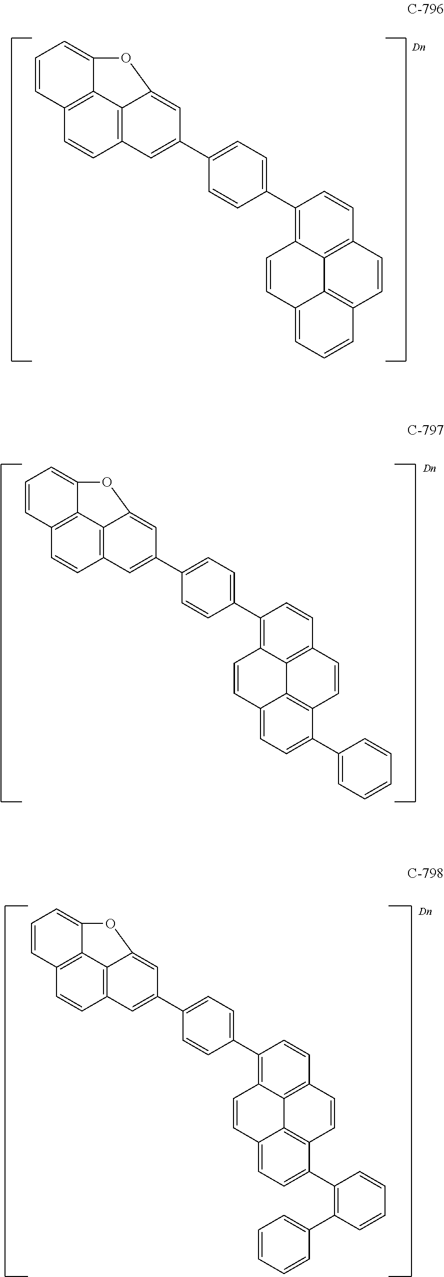 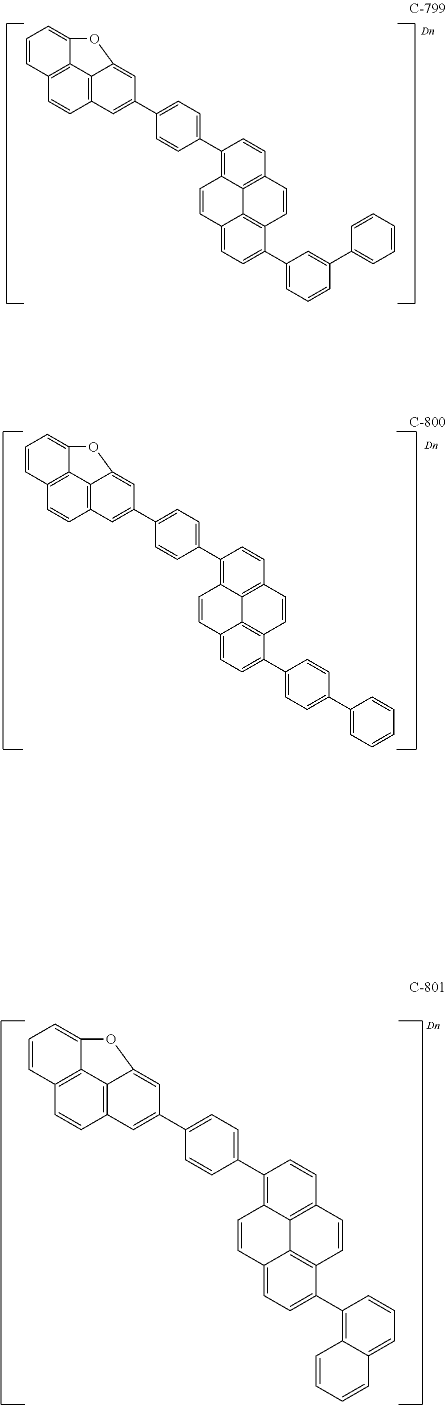 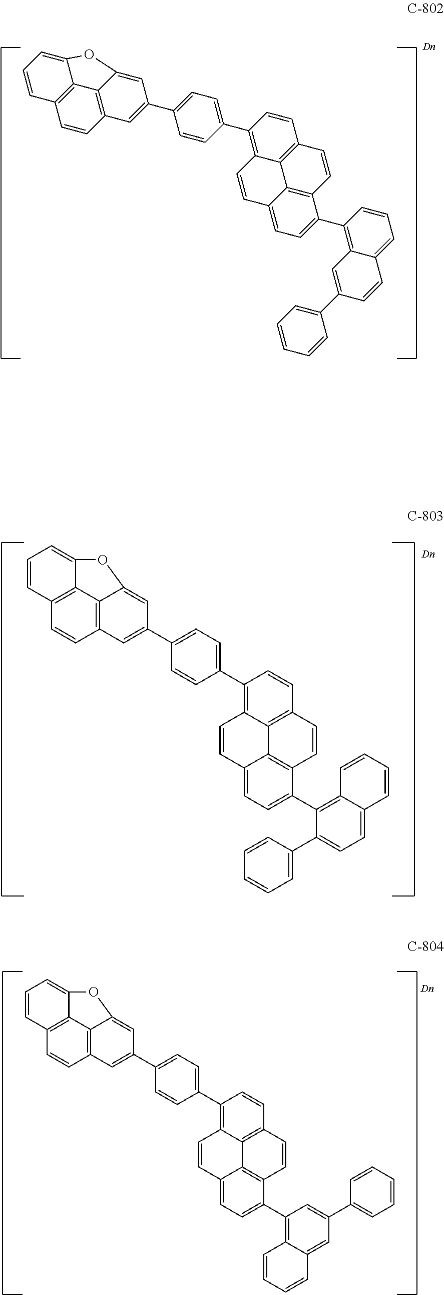 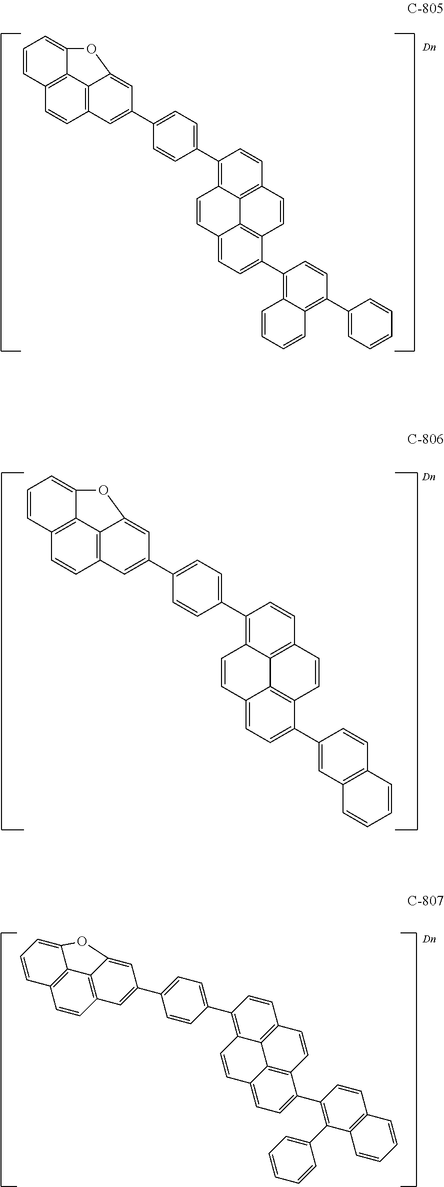 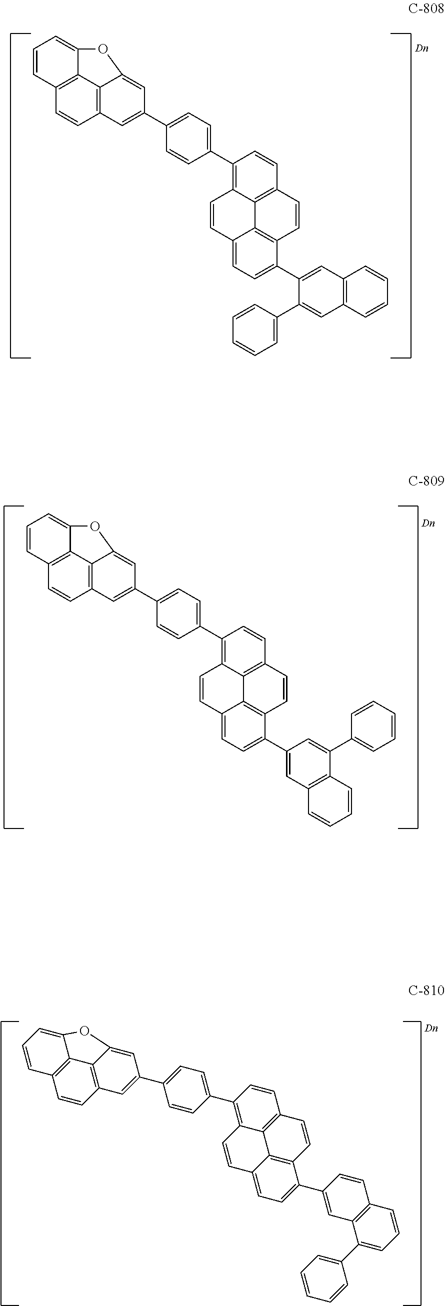 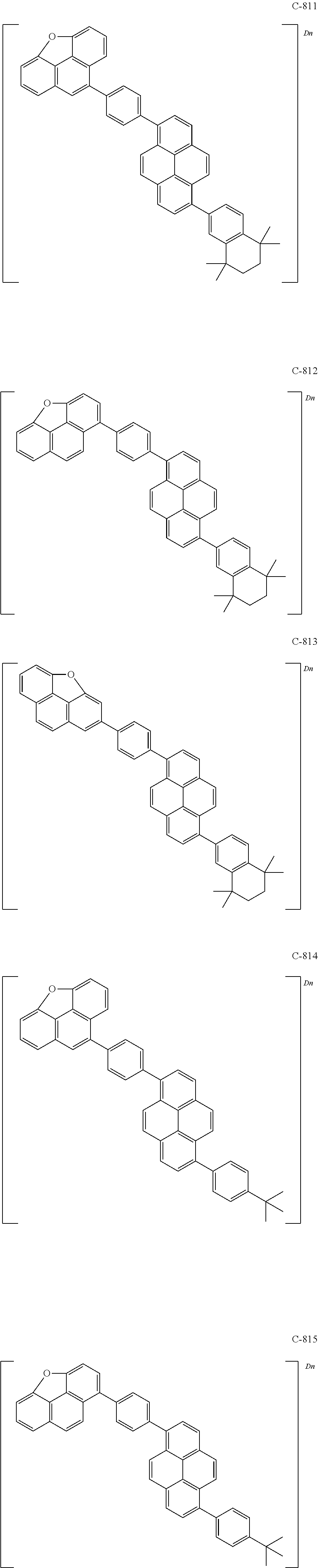 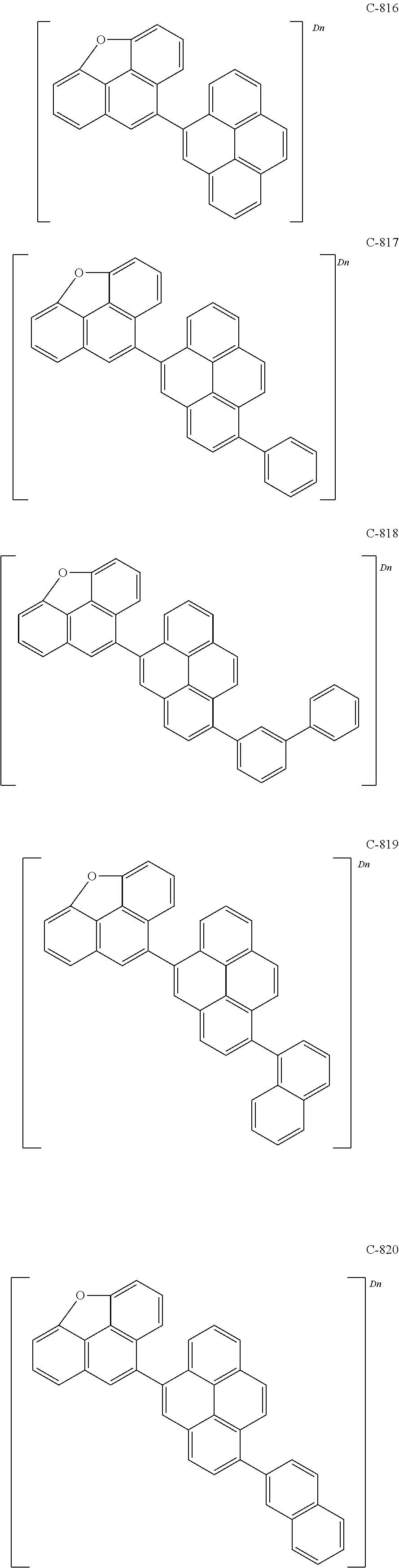 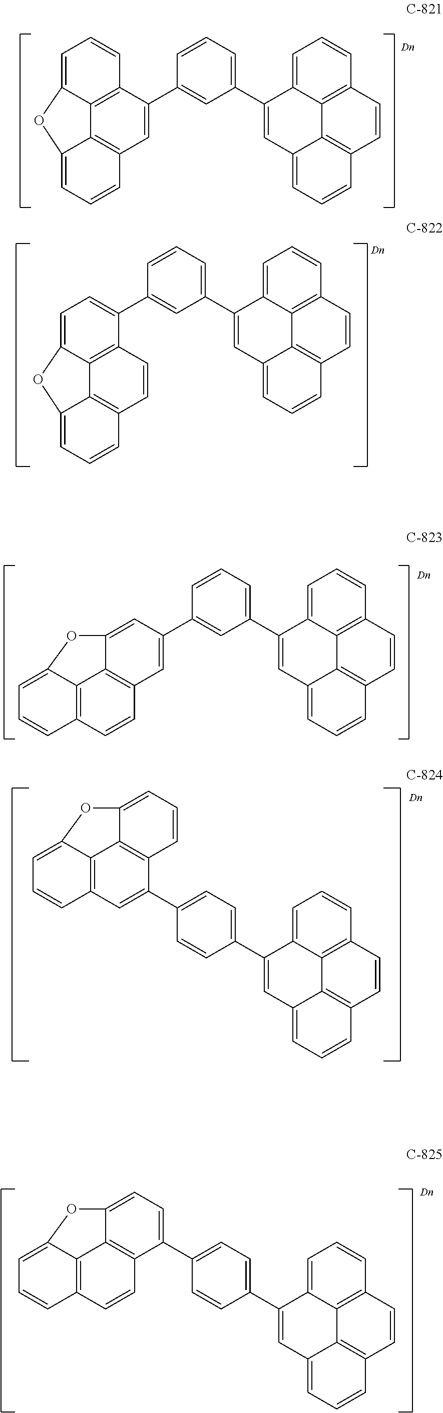 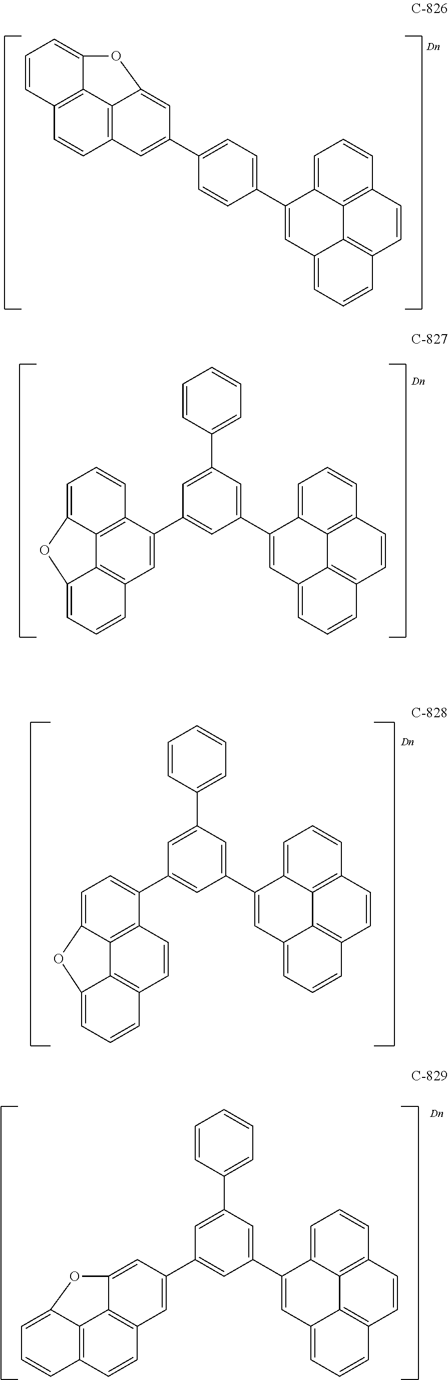 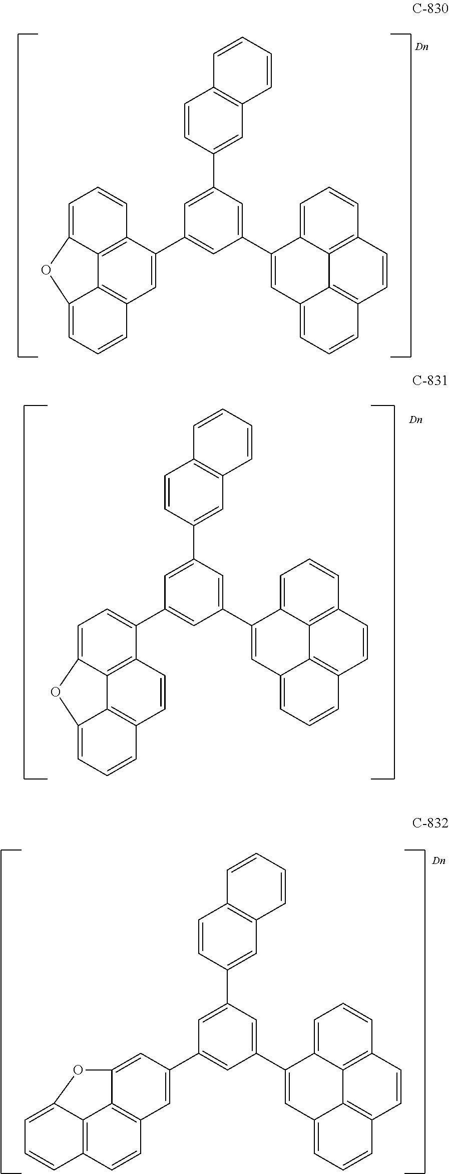 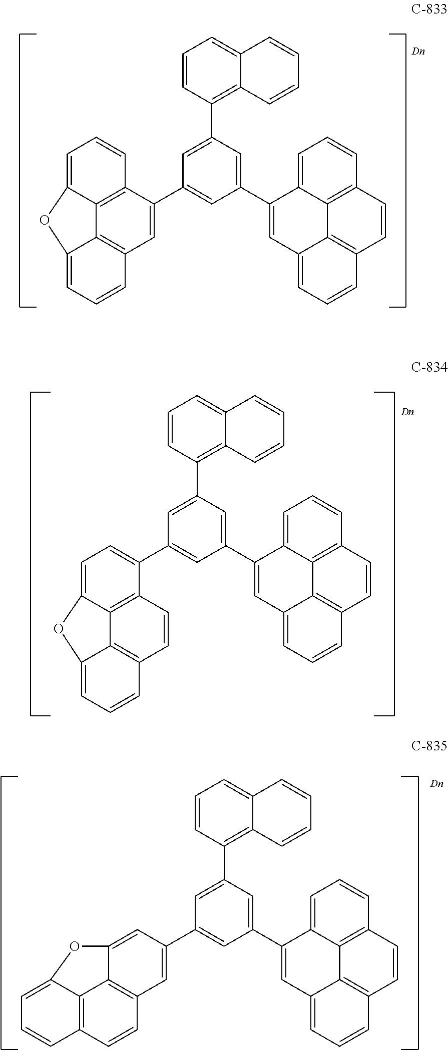 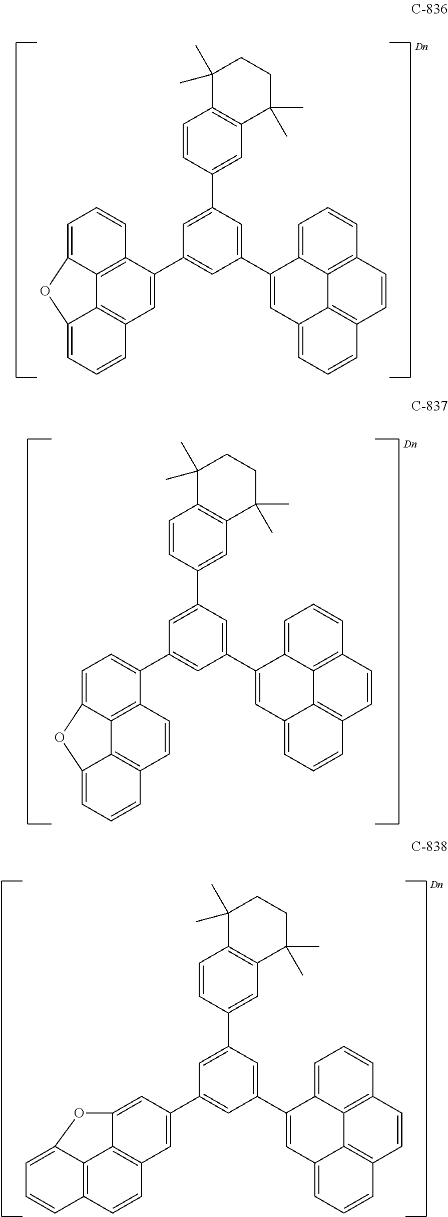 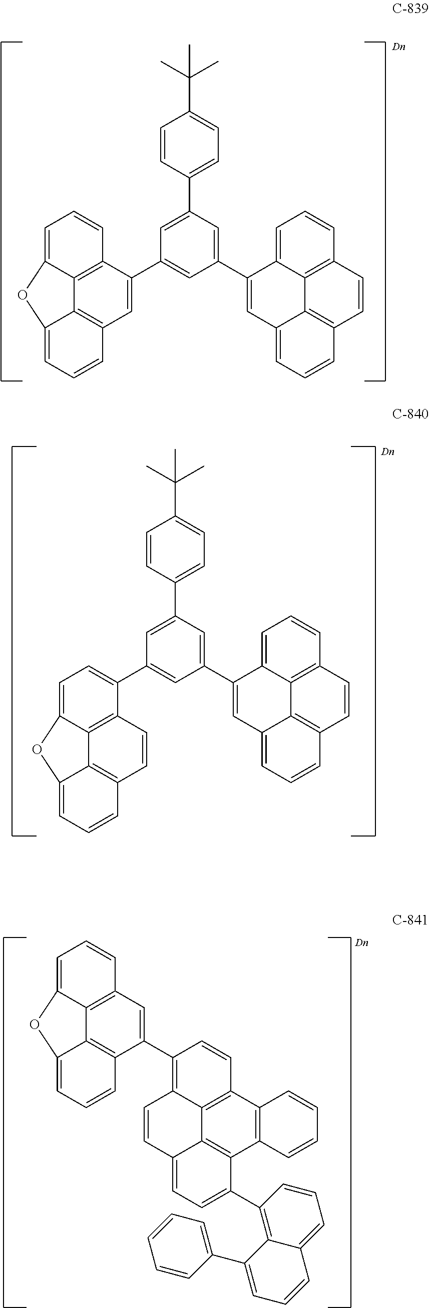 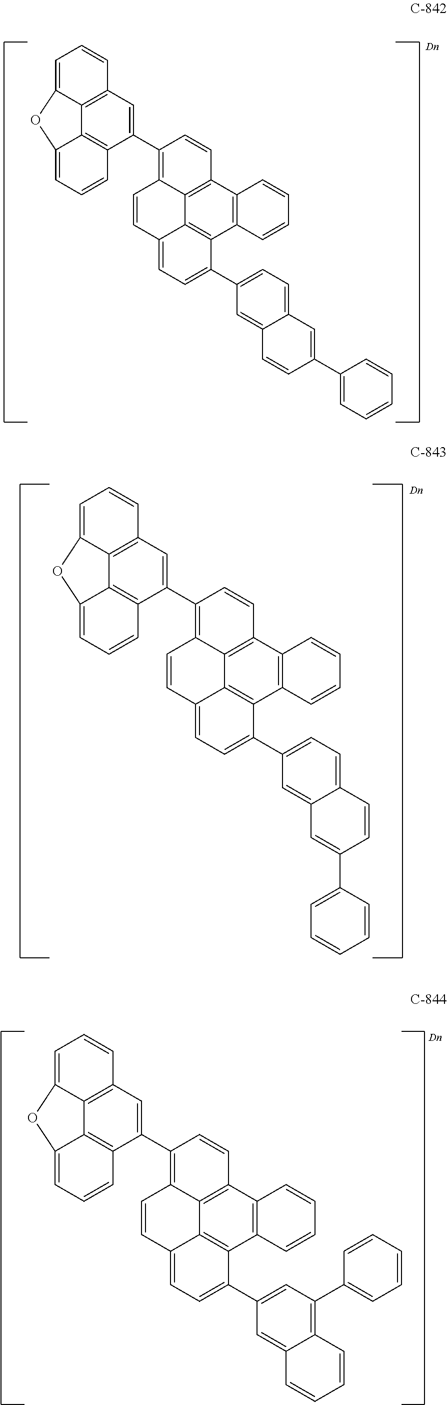 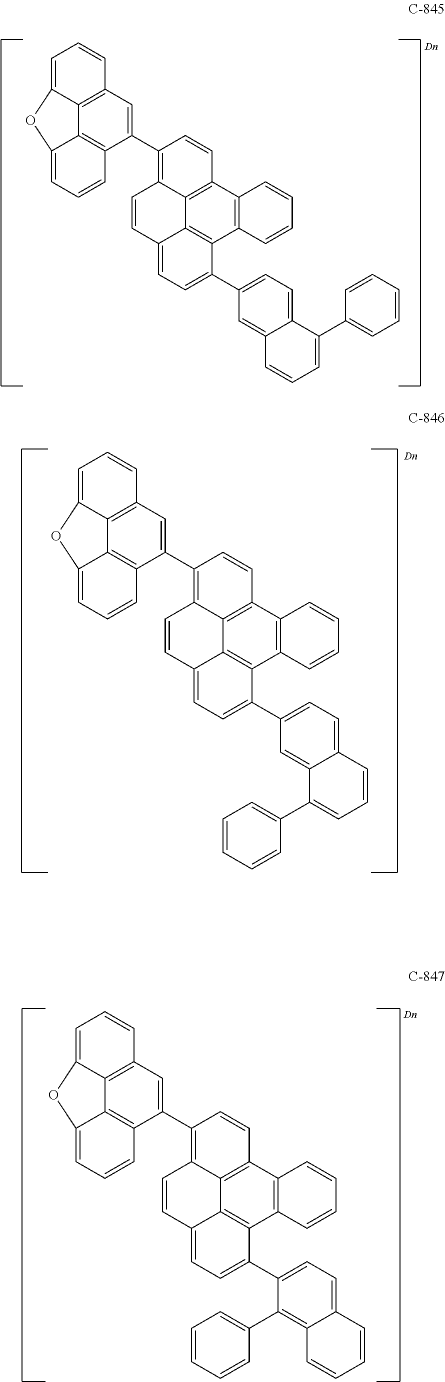 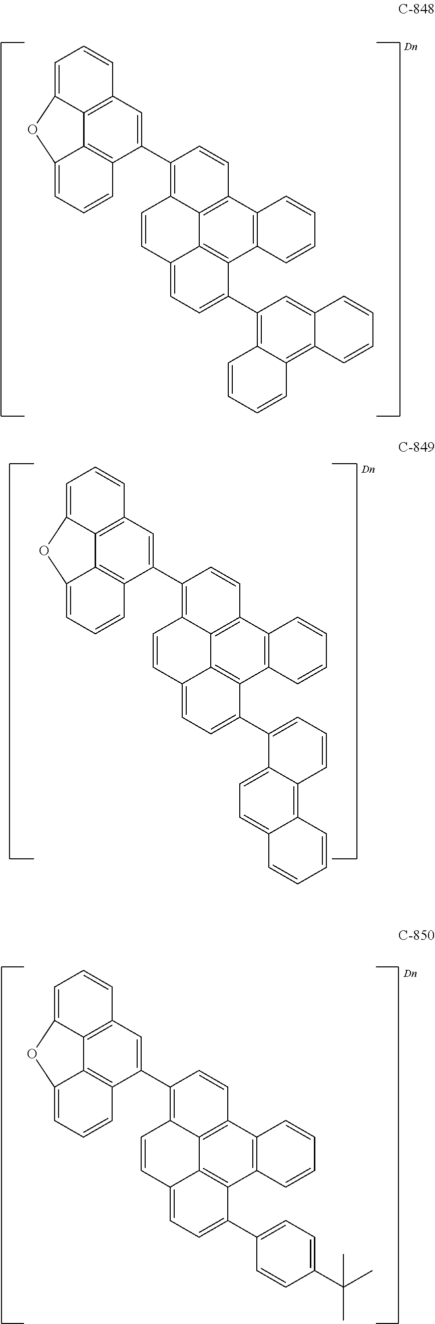 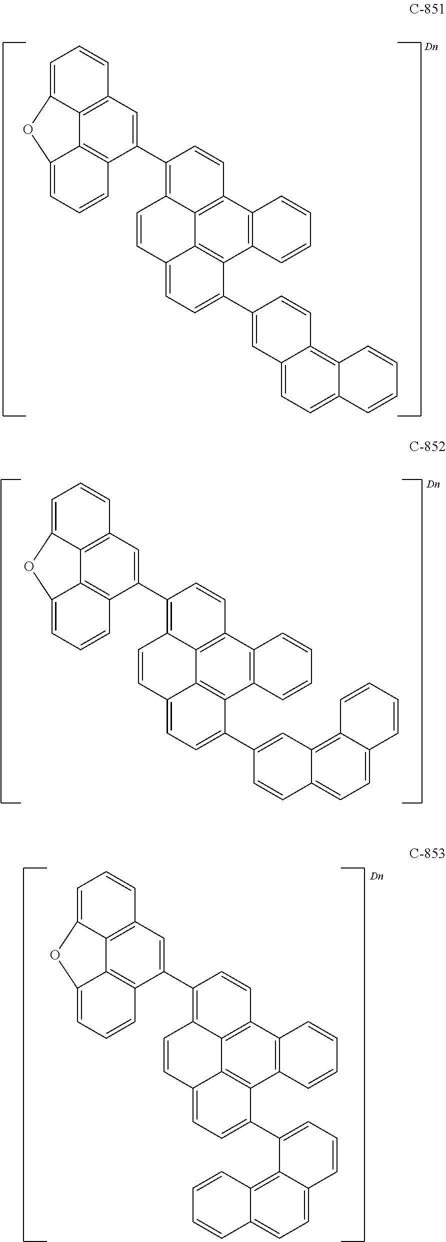 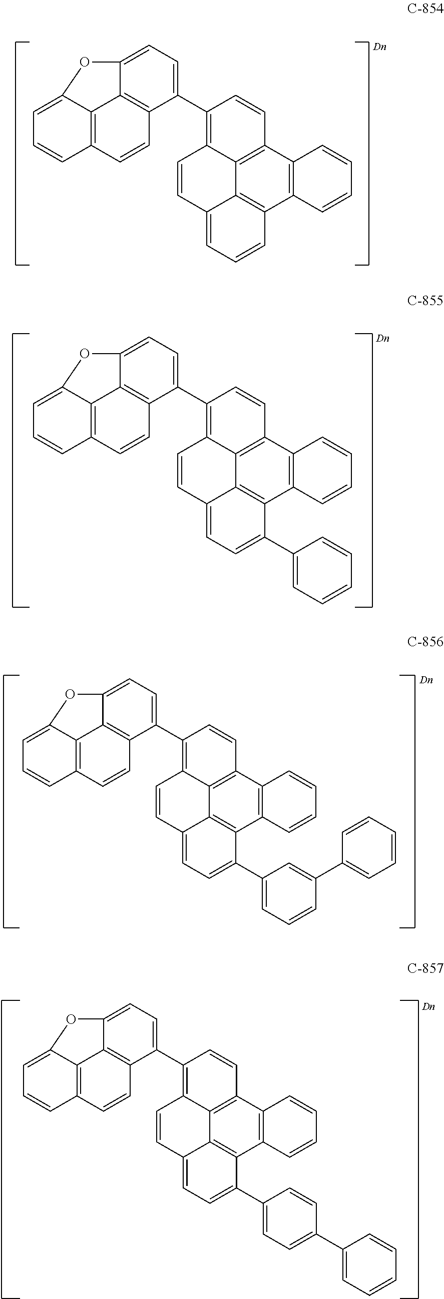 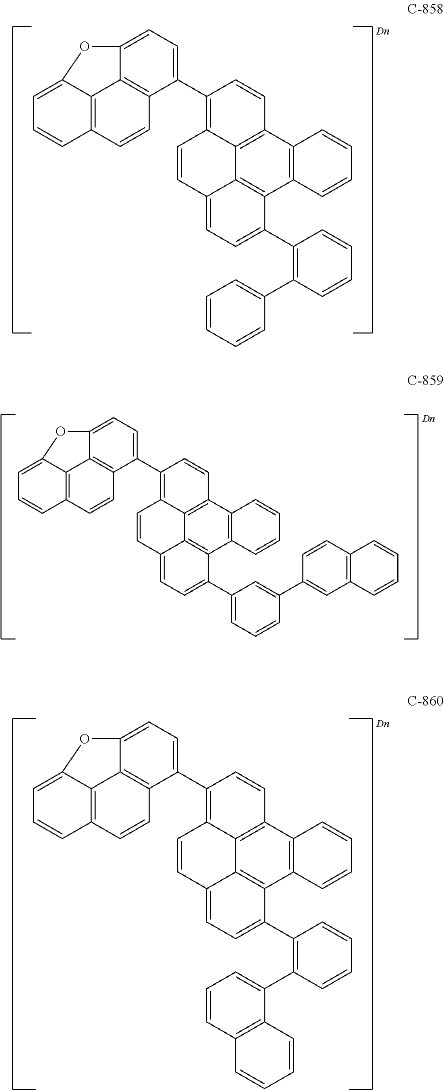 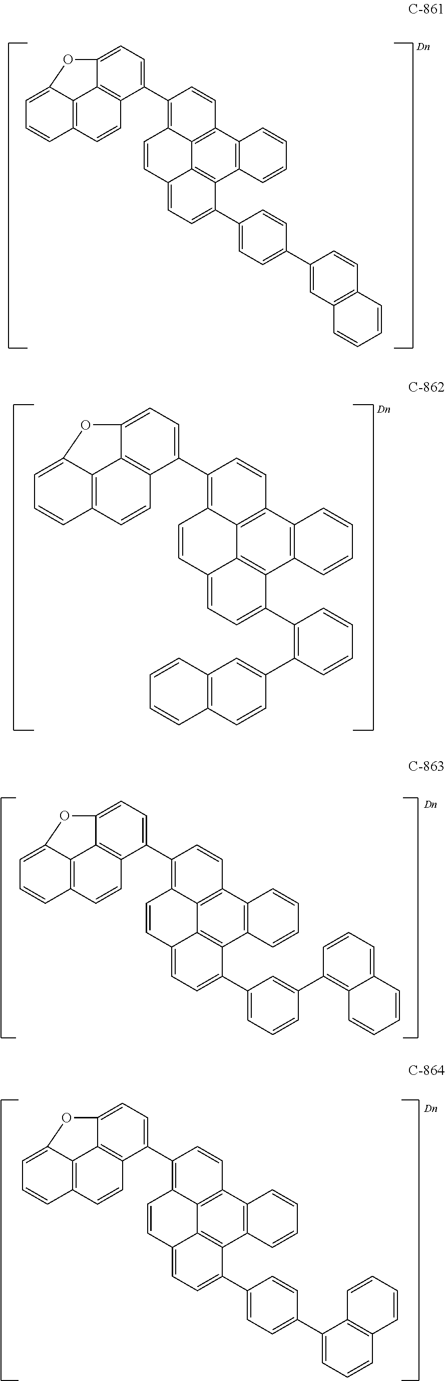 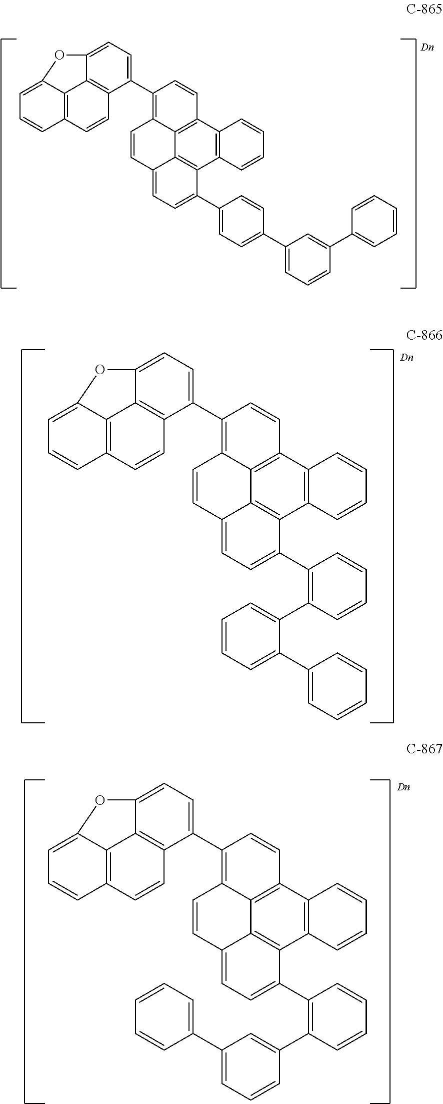 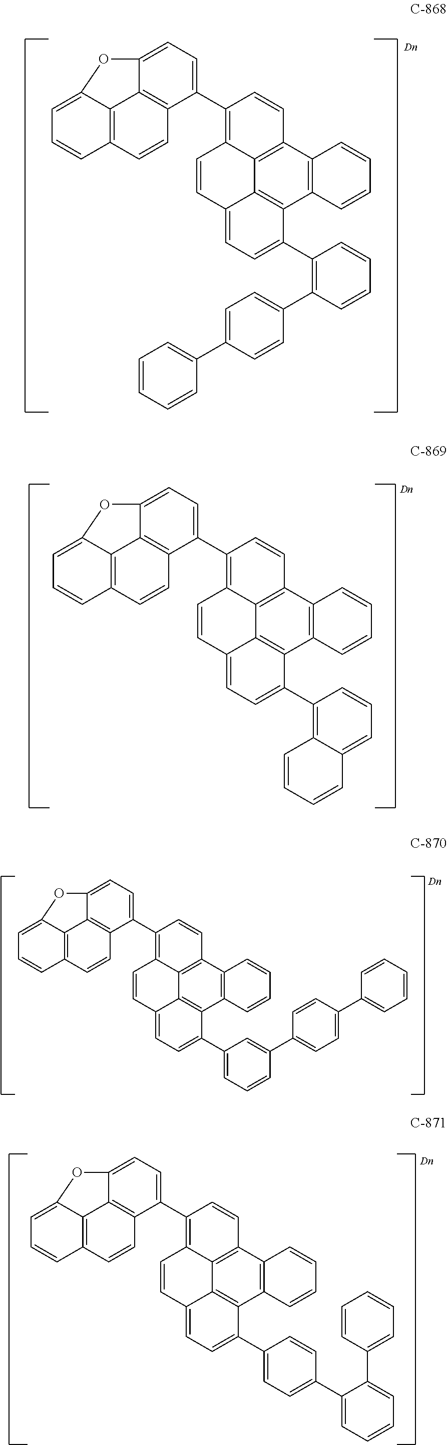 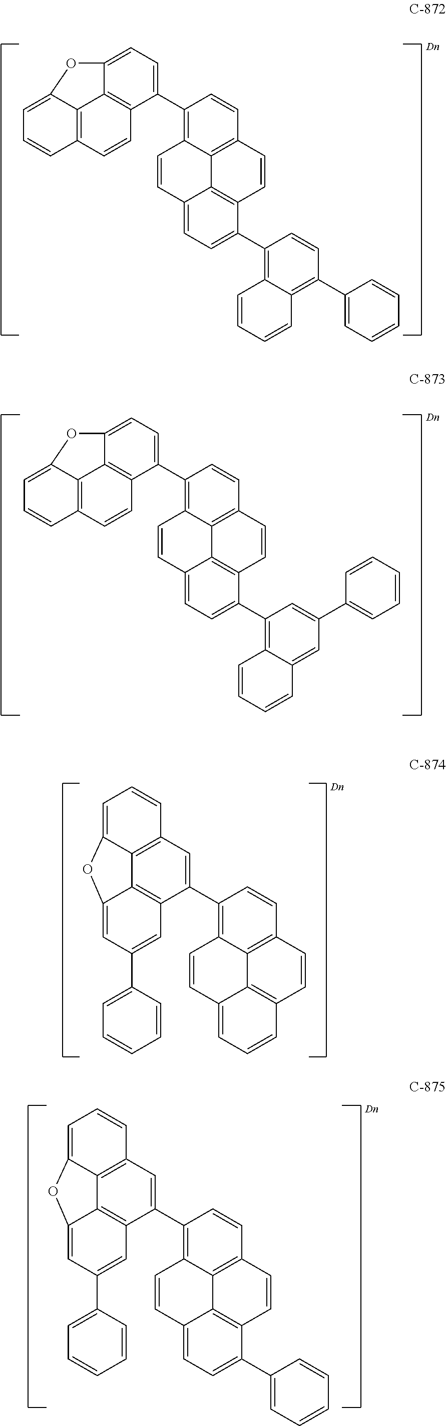 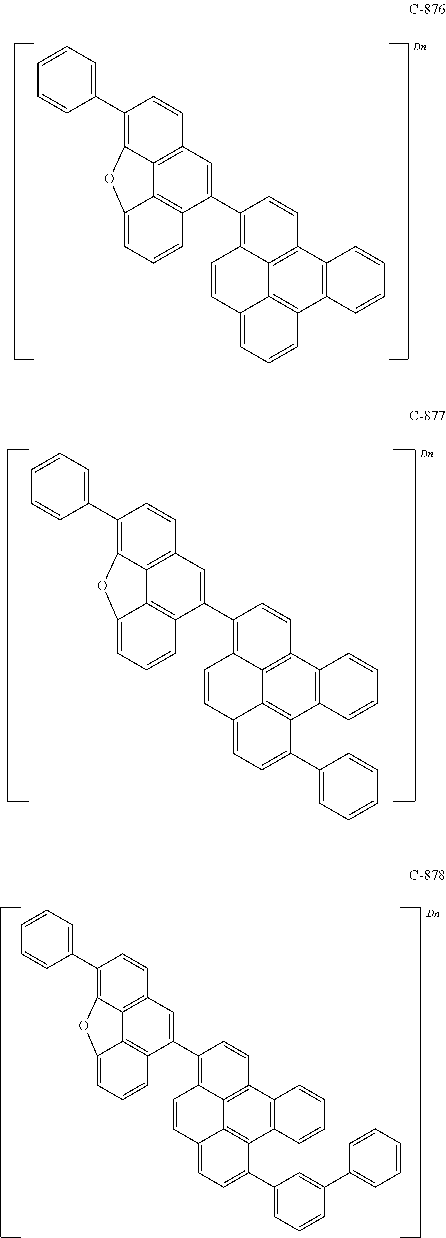 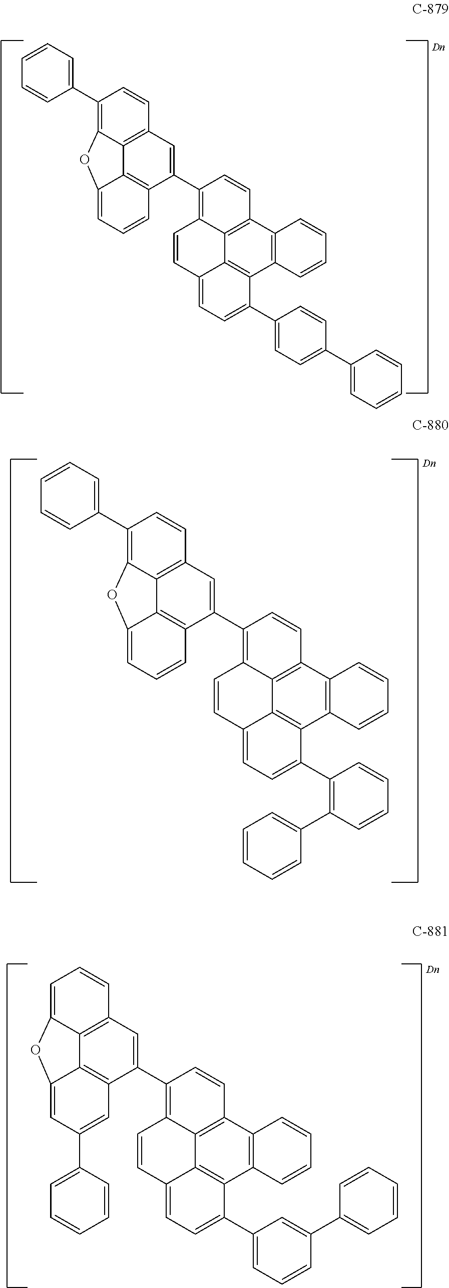 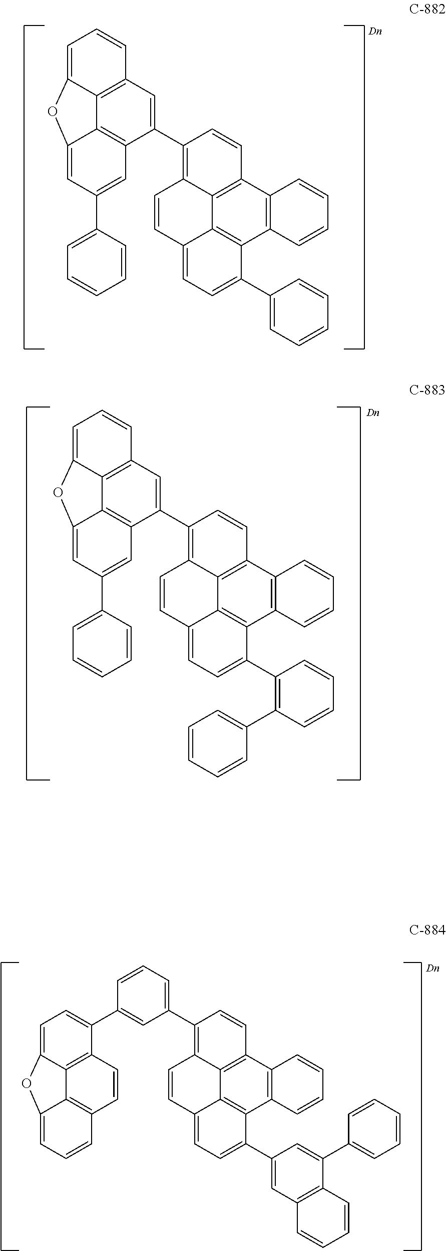 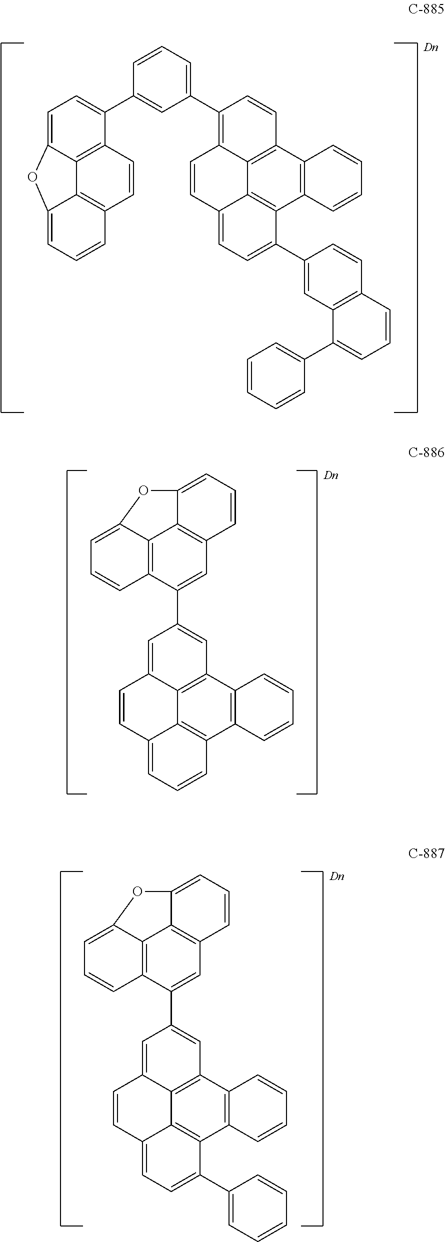 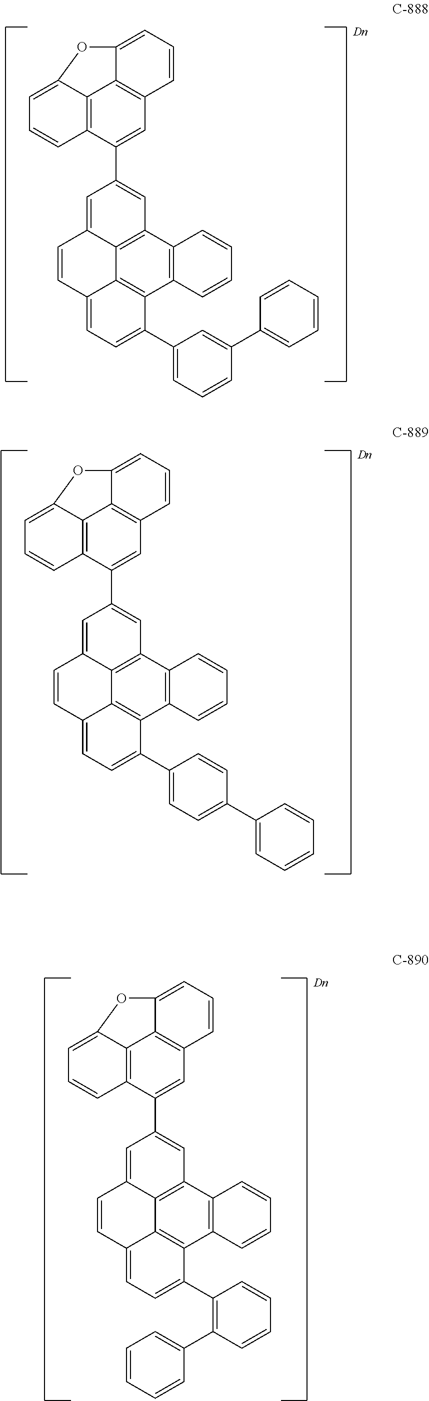 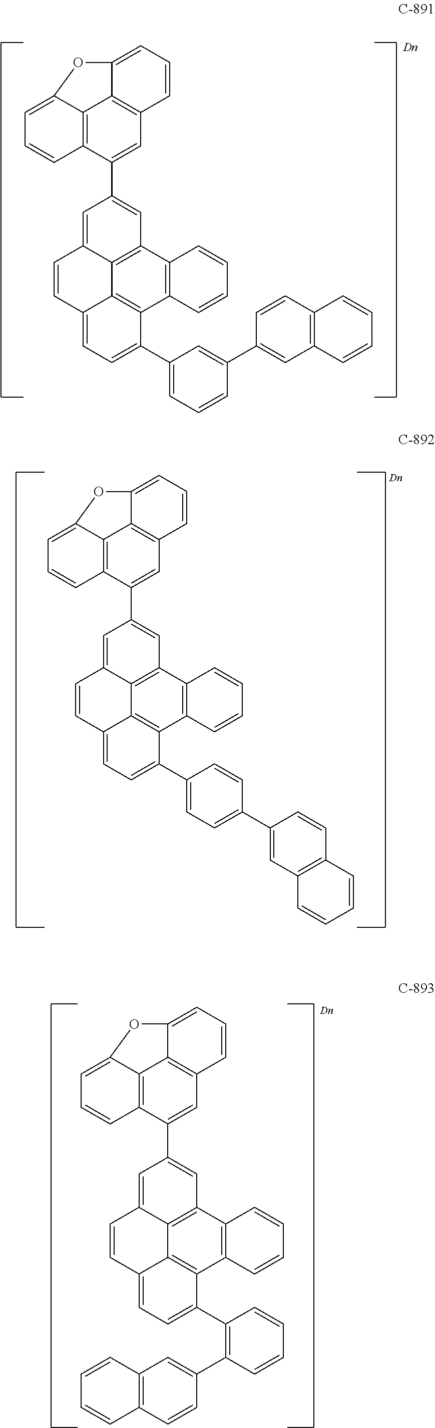 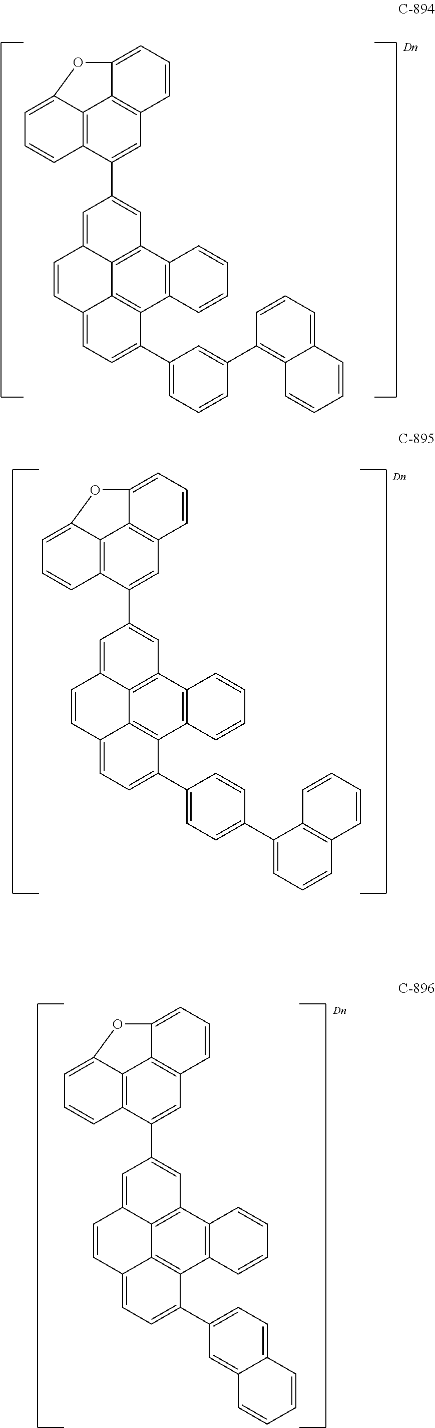 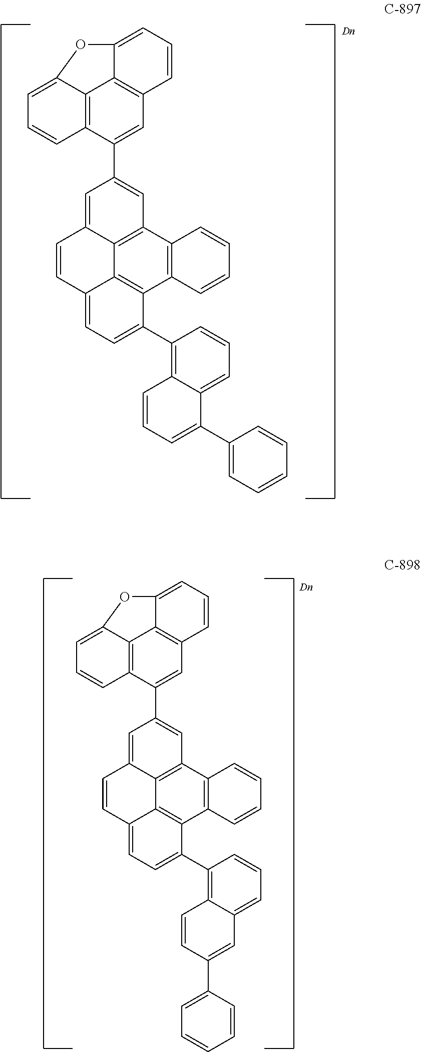 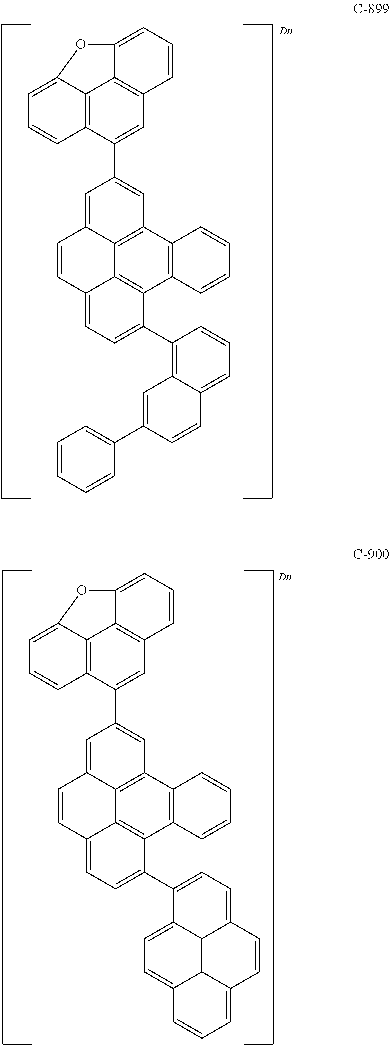 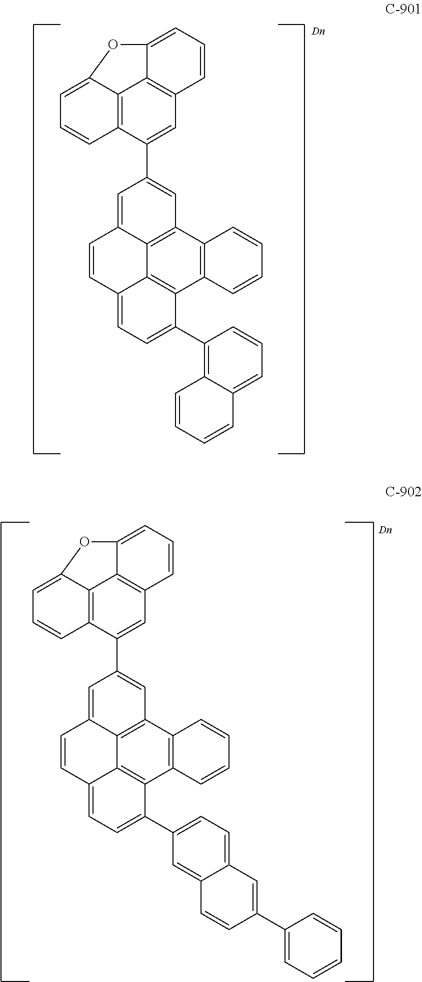 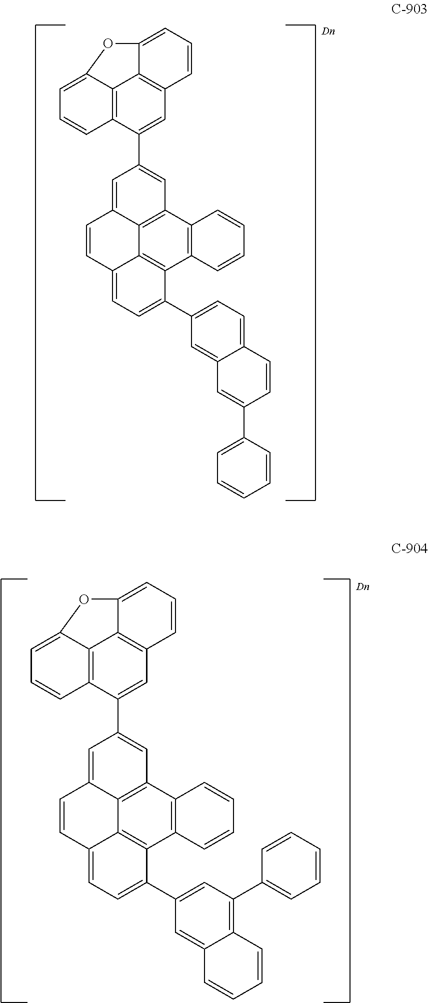 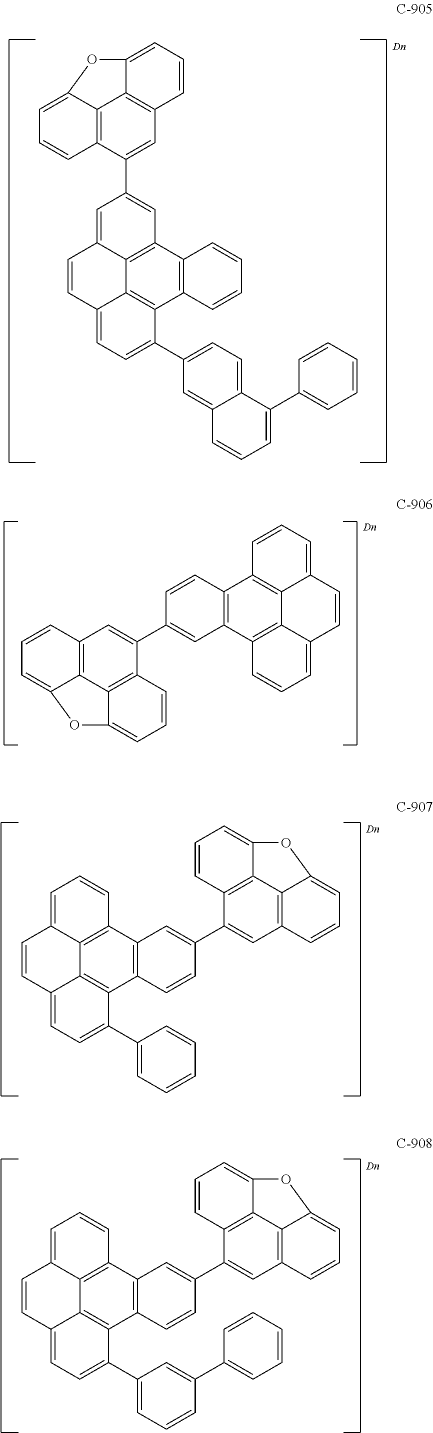 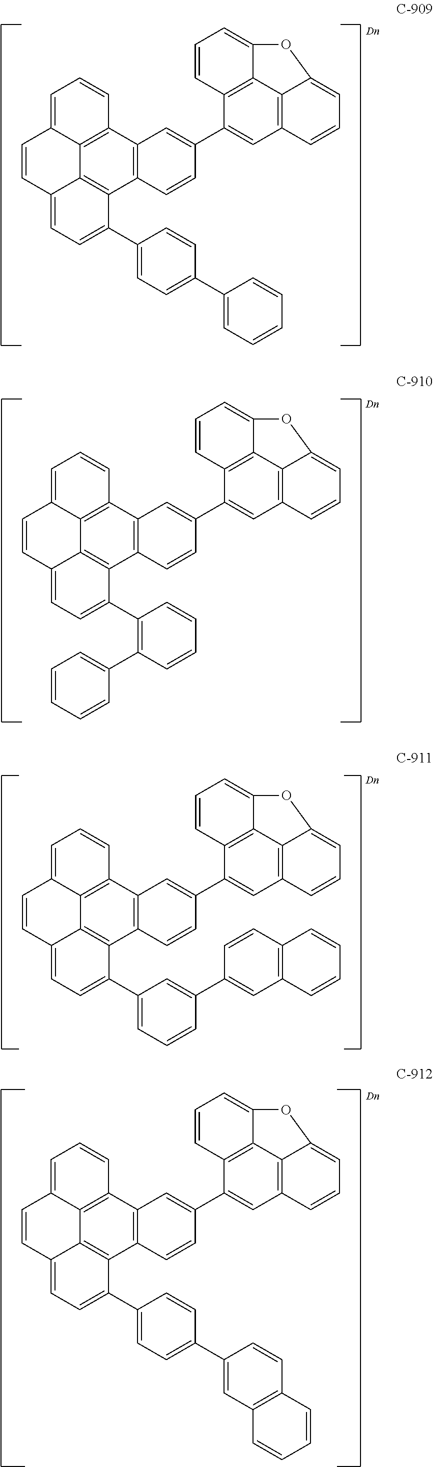 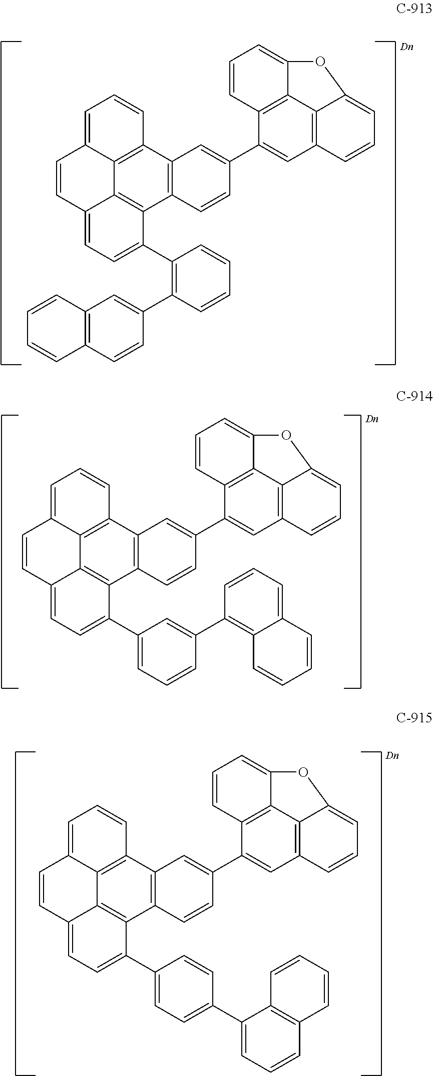 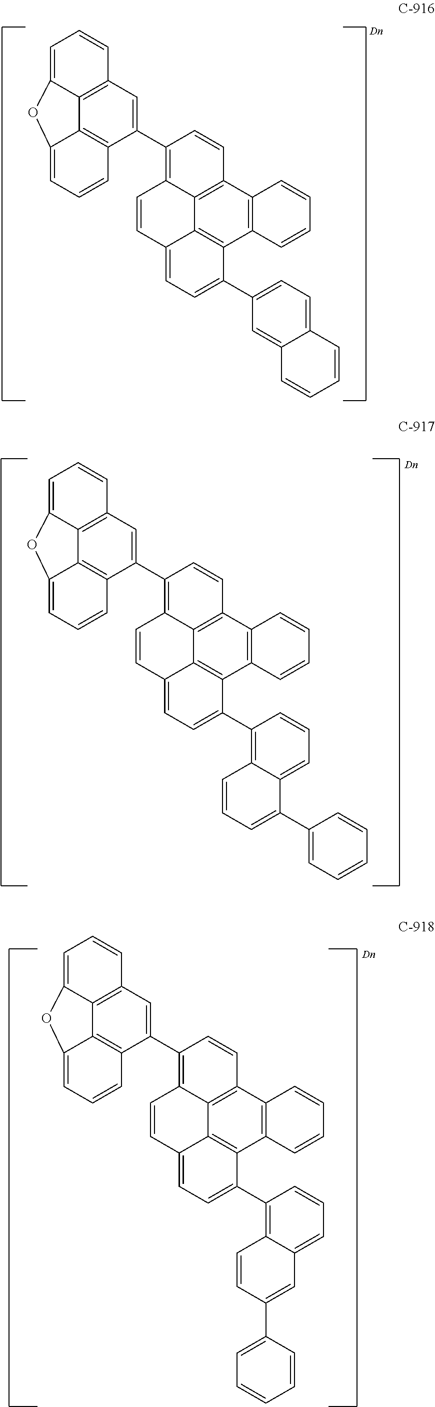 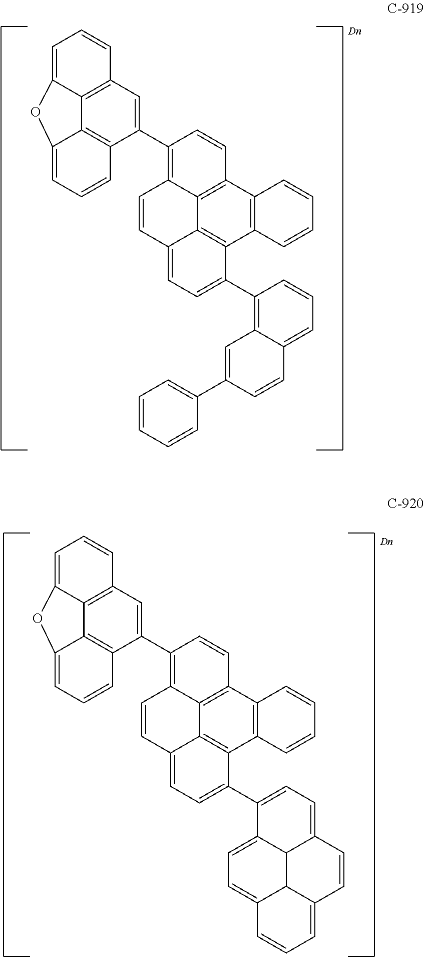 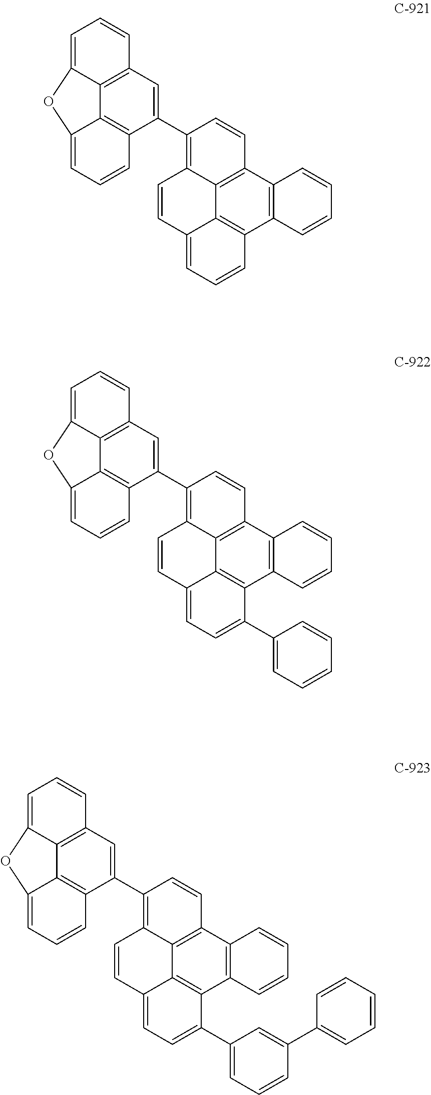 [SMILES: c1cc2cc(-c3ccc4c5ccccc5c5cccc6ccc3c4c65)c3cccc4oc(c1)c2c43 ; c1ccc(-c2ccc3ccc4c(-c5cc6cccc7oc8cccc5c8c67)ccc5c6ccccc6c2c3c45)cc1 ; c1ccc(-c2cccc(-c3ccc4ccc5c(-c6cc7cccc8oc9cccc6c9c78)ccc6c7ccccc7c3c4c56)c2)cc1] 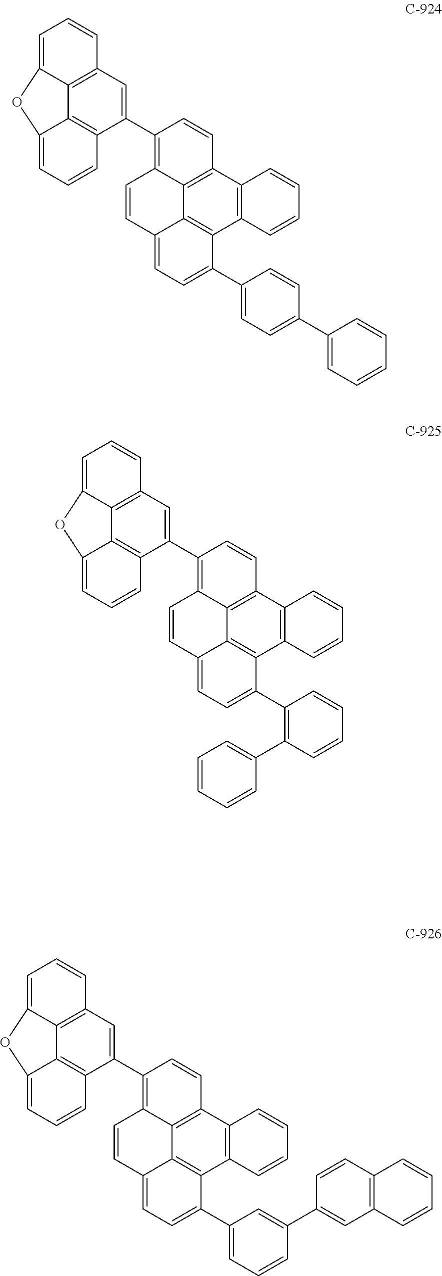 [SMILES: c1ccc(-c2ccc(-c3ccc4ccc5c(-c6cc7cccc8oc9cccc6c9c78)ccc6c7ccccc7c3c4c56)cc2)cc1 ; c1ccc(-c2ccccc2-c2ccc3ccc4c(-c5cc6cccc7oc8cccc5c8c67)ccc5c6ccccc6c2c3c45)cc1 ; c1cc(-c2ccc3ccccc3c2)cc(-c2ccc3ccc4c(-c5cc6cccc7oc8cccc5c8c67)ccc5c6ccccc6c2c3c45)c1] 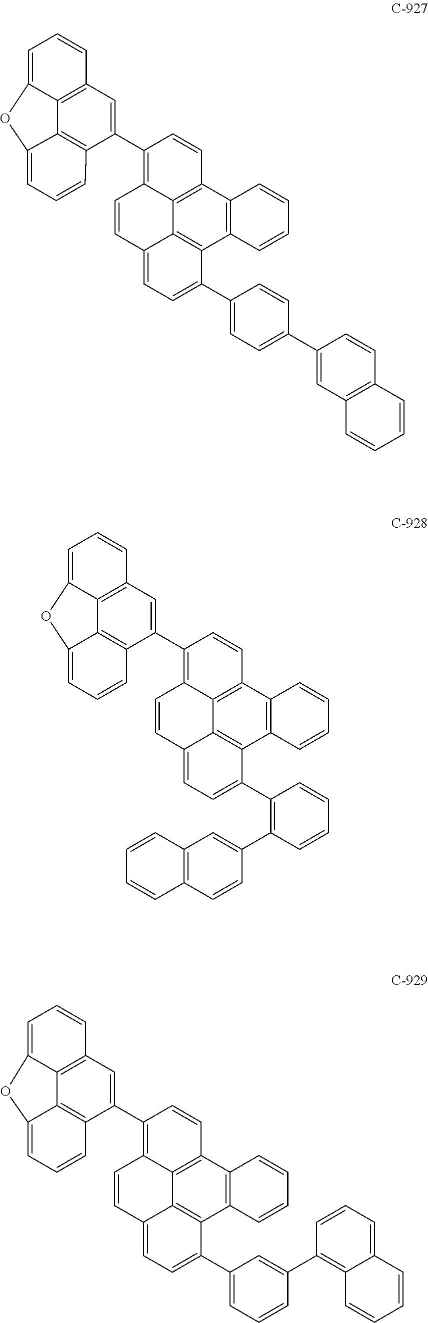 [SMILES: c1ccc2cc(-c3ccc(-c4ccc5ccc6c(-c7cc8cccc9oc%10cccc7c%10c89)ccc7c8ccccc8c4c5c67)cc3)ccc2c1 ; c1ccc(-c2ccc3ccc4c(-c5cc6cccc7oc8cccc5c8c67)ccc5c6ccccc6c2c3c45)c(-c2ccc3ccccc3c2)c1 ; c1cc(-c2cccc3ccccc23)cc(-c2ccc3ccc4c(-c5cc6cccc7oc8cccc5c8c67)ccc5c6ccccc6c2c3c45)c1] 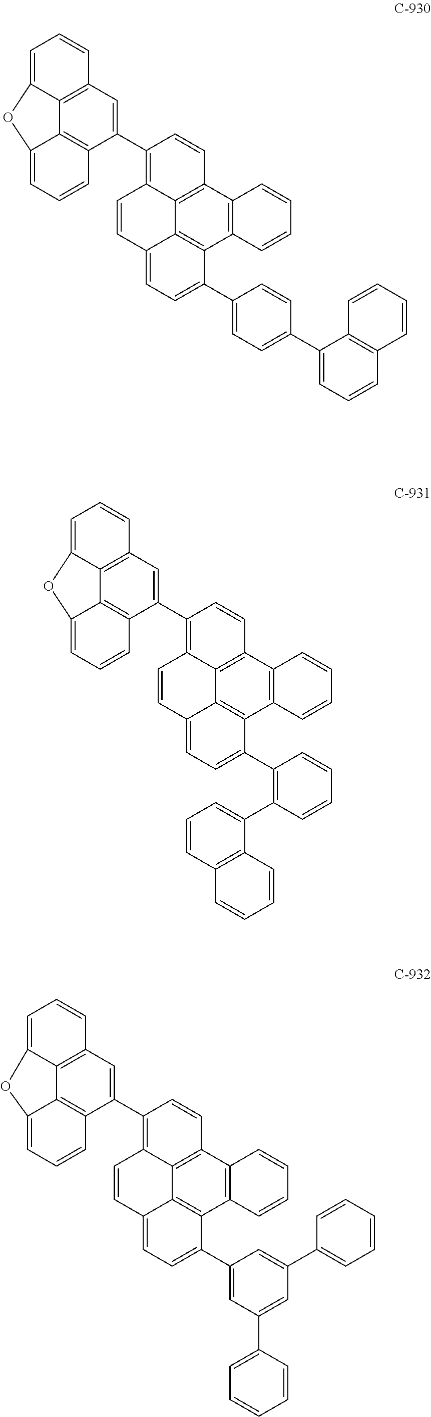 [SMILES: c1ccc2c(-c3ccc(-c4ccc5ccc6c(-c7cc8cccc9oc%10cccc7c%10c89)ccc7c8ccccc8c4c5c67)cc3)cccc2c1 ; c1ccc(-c2ccc3ccc4c(-c5cc6cccc7oc8cccc5c8c67)ccc5c6ccccc6c2c3c45)c(-c2cccc3ccccc23)c1 ; c1ccc(-c2cc(-c3ccccc3)cc(-c3ccc4ccc5c(-c6cc7cccc8oc9cccc6c9c78)ccc6c7ccccc7c3c4c56)c2)cc1] 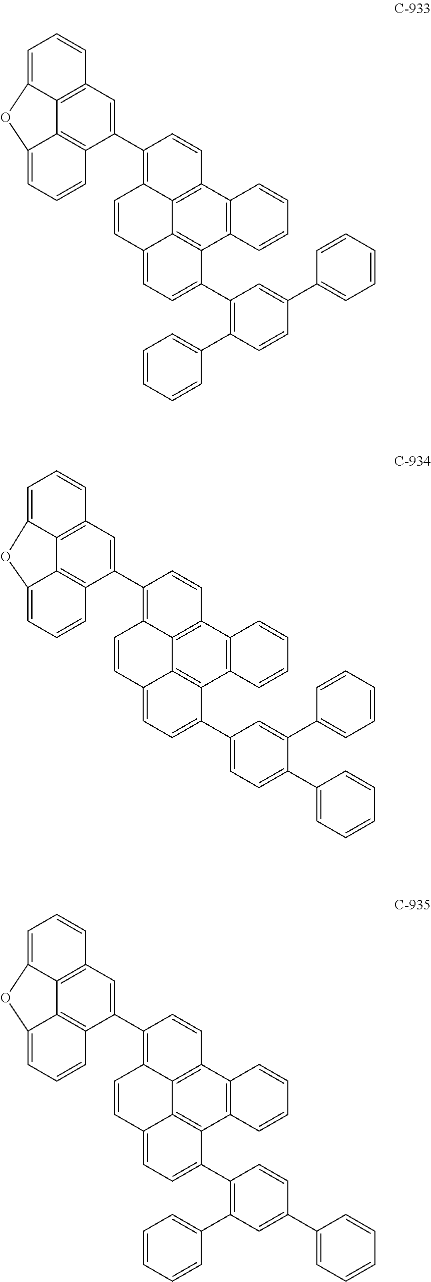 [SMILES: c1ccc(-c2ccc(-c3ccccc3)c(-c3ccc4ccc5c(-c6cc7cccc8oc9cccc6c9c78)ccc6c7ccccc7c3c4c56)c2)cc1 ; c1ccc(-c2ccc(-c3ccc4ccc5c(-c6cc7cccc8oc9cccc6c9c78)ccc6c7ccccc7c3c4c56)cc2-c2ccccc2)cc1 ; c1ccc(-c2ccc(-c3ccc4ccc5c(-c6cc7cccc8oc9cccc6c9c78)ccc6c7ccccc7c3c4c56)c(-c3ccccc3)c2)cc1] 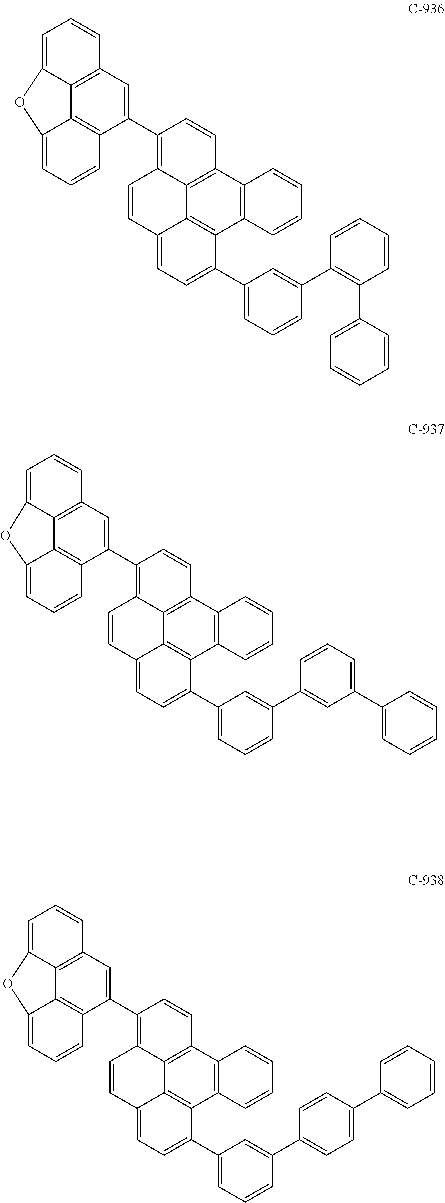 [SMILES: c1ccc(-c2ccccc2-c2cccc(-c3ccc4ccc5c(-c6cc7cccc8oc9cccc6c9c78)ccc6c7ccccc7c3c4c56)c2)cc1 ; c1ccc(-c2cccc(-c3cccc(-c4ccc5ccc6c(-c7cc8cccc9oc%10cccc7c%10c89)ccc7c8ccccc8c4c5c67)c3)c2)cc1 ; c1ccc(-c2ccc(-c3cccc(-c4ccc5ccc6c(-c7cc8cccc9oc%10cccc7c%10c89)ccc7c8ccccc8c4c5c67)c3)cc2)cc1] 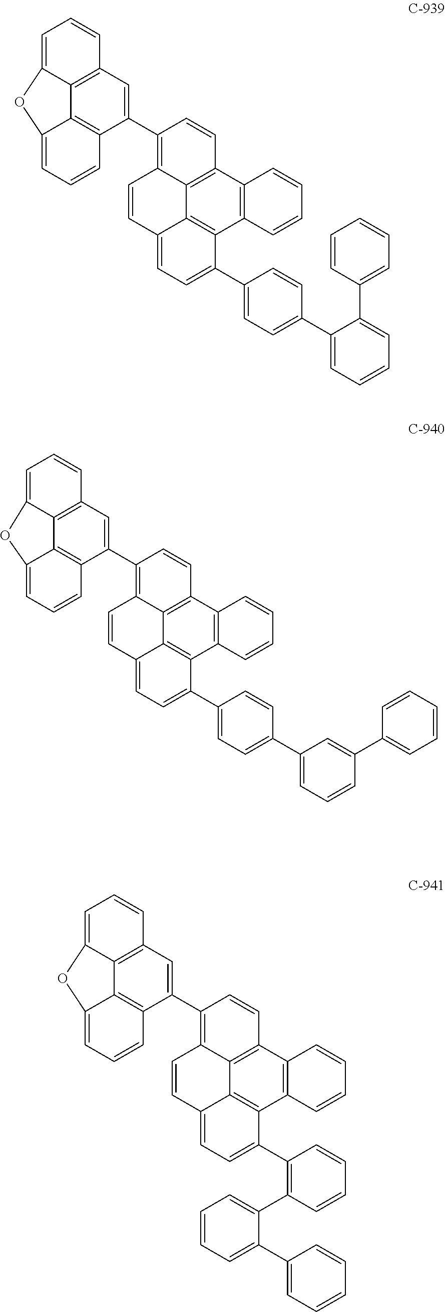 [SMILES: c1ccc(-c2ccccc2-c2ccc(-c3ccc4ccc5c(-c6cc7cccc8oc9cccc6c9c78)ccc6c7ccccc7c3c4c56)cc2)cc1 ; c1ccc(-c2cccc(-c3ccc(-c4ccc5ccc6c(-c7cc8cccc9oc%10cccc7c%10c89)ccc7c8ccccc8c4c5c67)cc3)c2)cc1 ; c1ccc(-c2ccccc2-c2ccccc2-c2ccc3ccc4c(-c5cc6cccc7oc8cccc5c8c67)ccc5c6ccccc6c2c3c45)cc1] 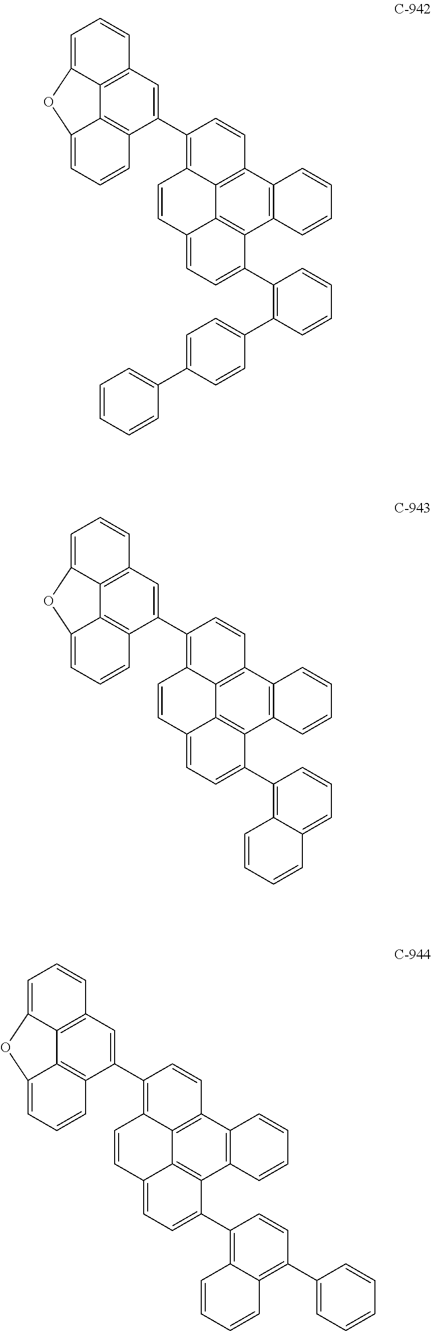 [SMILES: c1ccc(-c2ccc(-c3ccccc3-c3ccc4ccc5c(-c6cc7cccc8oc9cccc6c9c78)ccc6c7ccccc7c3c4c56)cc2)cc1 ; c1ccc2c(-c3ccc4ccc5c(-c6cc7cccc8oc9cccc6c9c78)ccc6c7ccccc7c3c4c56)cccc2c1 ; c1ccc(-c2ccc(-c3ccc4ccc5c(-c6cc7cccc8oc9cccc6c9c78)ccc6c7ccccc7c3c4c56)c3ccccc23)cc1] 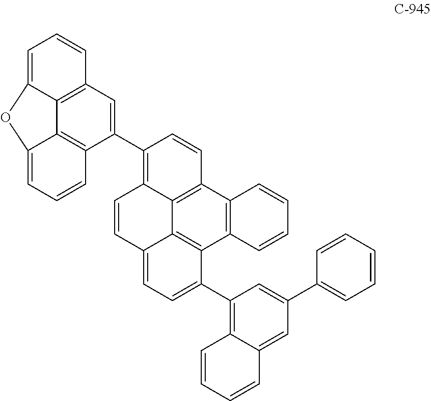 [SMILES: c1ccc(-c2cc(-c3ccc4ccc5c(-c6cc7cccc8oc9cccc6c9c78)ccc6c7ccccc7c3c4c56)c3ccccc3c2)cc1] 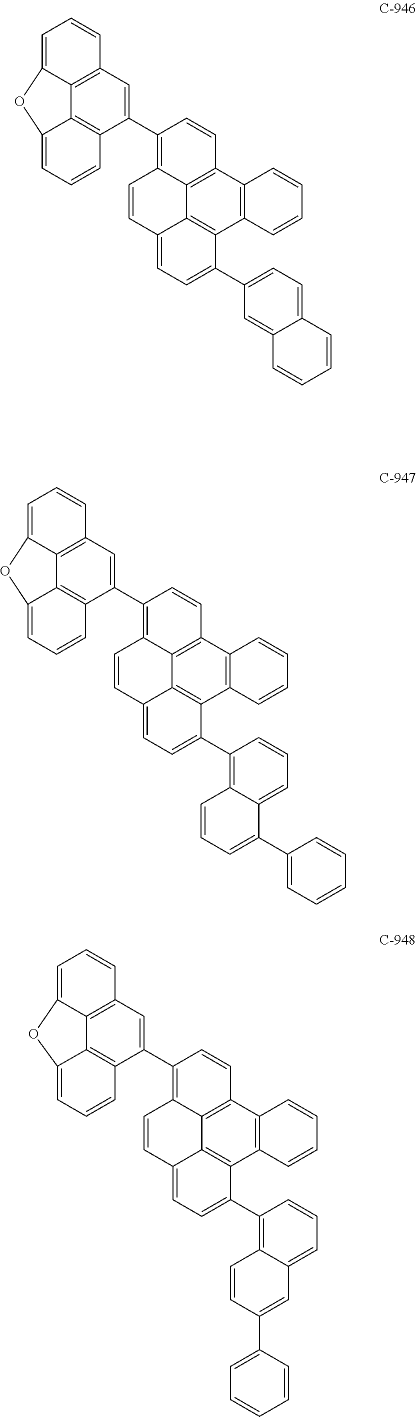 [SMILES: c1ccc2cc(-c3ccc4ccc5c(-c6cc7cccc8oc9cccc6c9c78)ccc6c7ccccc7c3c4c56)ccc2c1 ; c1ccc(-c2cccc3c(-c4ccc5ccc6c(-c7cc8cccc9oc%10cccc7c%10c89)ccc7c8ccccc8c4c5c67)cccc23)cc1 ; c1ccc(-c2ccc3c(-c4ccc5ccc6c(-c7cc8cccc9oc%10cccc7c%10c89)ccc7c8ccccc8c4c5c67)cccc3c2)cc1] 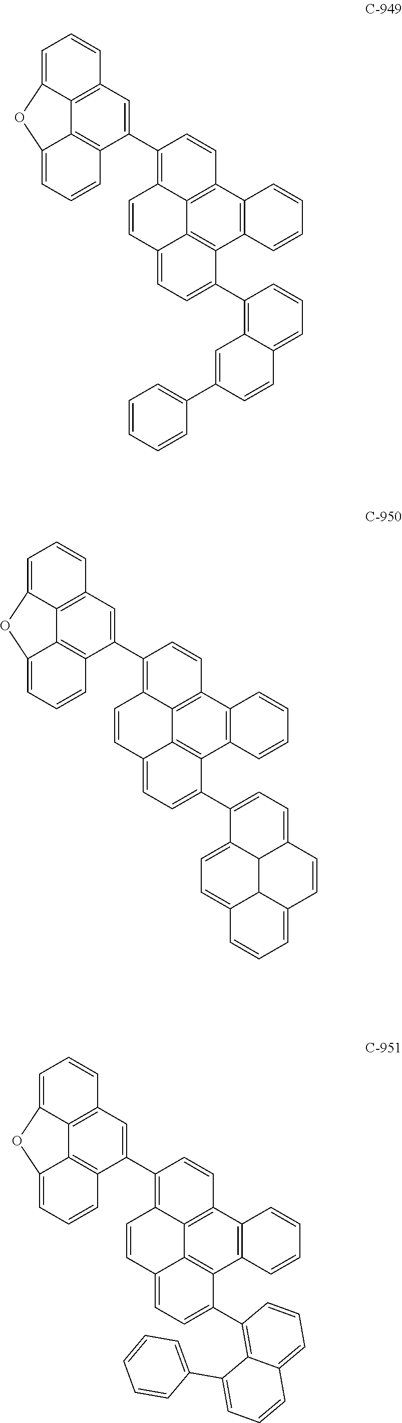 [SMILES: c1ccc(-c2ccc3cccc(-c4ccc5ccc6c(-c7cc8cccc9oc%10cccc7c%10c89)ccc7c8ccccc8c4c5c67)c3c2)cc1 ; C1=CC2=CC=C3C(c4ccc5ccc6c(-c7cc8cccc9oc%10cccc7c%10c89)ccc7c8ccccc8c4c5c67)=CC=C4C=CC(=C1)C2C43 ; c1ccc(-c2cccc3cccc(-c4ccc5ccc6c(-c7cc8cccc9oc%10cccc7c%10c89)ccc7c8ccccc8c4c5c67)c23)cc1] 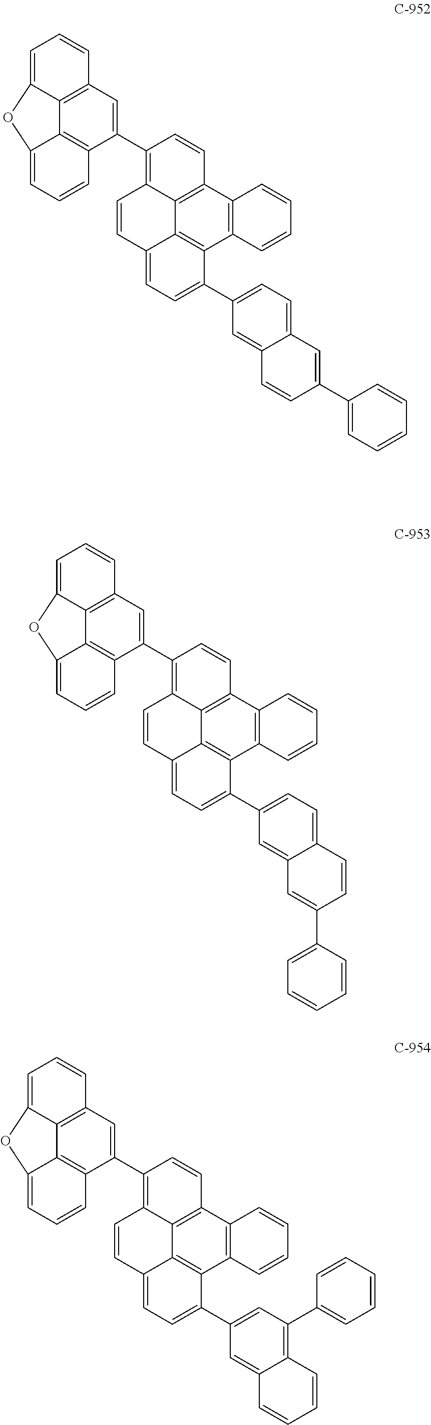 [SMILES: c1ccc(-c2ccc3cc(-c4ccc5ccc6c(-c7cc8cccc9oc%10cccc7c%10c89)ccc7c8ccccc8c4c5c67)ccc3c2)cc1 ; c1ccc(-c2ccc3ccc(-c4ccc5ccc6c(-c7cc8cccc9oc%10cccc7c%10c89)ccc7c8ccccc8c4c5c67)cc3c2)cc1 ; c1ccc(-c2cc(-c3ccc4ccc5c(-c6cc7cccc8oc9cccc6c9c78)ccc6c7ccccc7c3c4c56)cc3ccccc23)cc1] 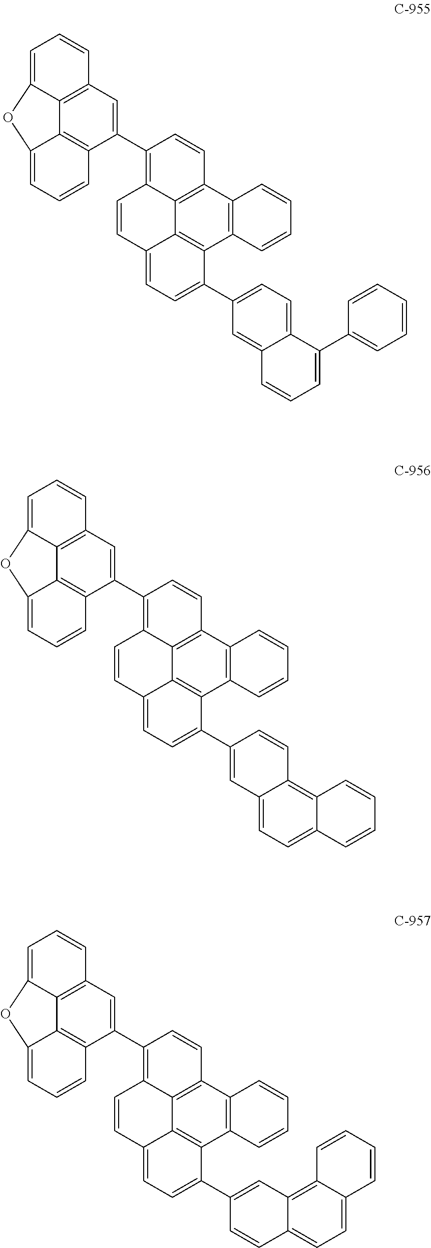 [SMILES: c1ccc(-c2cccc3cc(-c4ccc5ccc6c(-c7cc8cccc9oc%10cccc7c%10c89)ccc7c8ccccc8c4c5c67)ccc23)cc1 ; c1ccc2c(c1)ccc1cc(-c3ccc4ccc5c(-c6cc7cccc8oc9cccc6c9c78)ccc6c7ccccc7c3c4c56)ccc12 ; c1ccc2c(c1)ccc1ccc(-c3ccc4ccc5c(-c6cc7cccc8oc9cccc6c9c78)ccc6c7ccccc7c3c4c56)cc12] 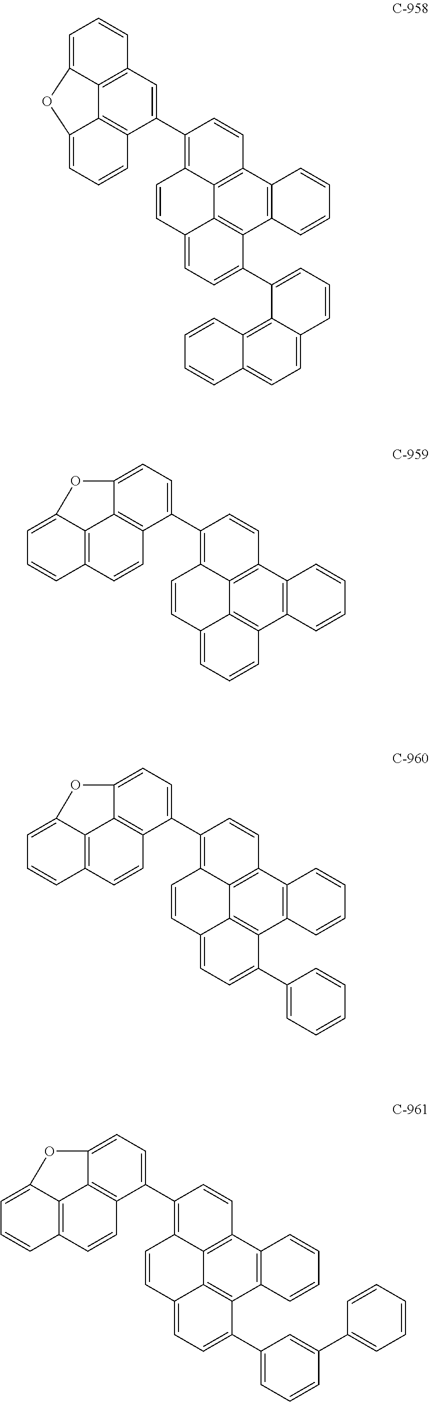 [SMILES: c1ccc2c(c1)ccc1cccc(-c3ccc4ccc5c(-c6cc7cccc8oc9cccc6c9c78)ccc6c7ccccc7c3c4c56)c12 ; c1cc2ccc3c(-c4ccc5c6ccccc6c6cccc7ccc4c5c76)ccc4oc(c1)c2c43 ; c1ccc(-c2ccc3ccc4c(-c5ccc6oc7cccc8ccc5c6c87)ccc5c6ccccc6c2c3c45)cc1 ; c1ccc(-c2cccc(-c3ccc4ccc5c(-c6ccc7oc8cccc9ccc6c7c98)ccc6c7ccccc7c3c4c56)c2)cc1] 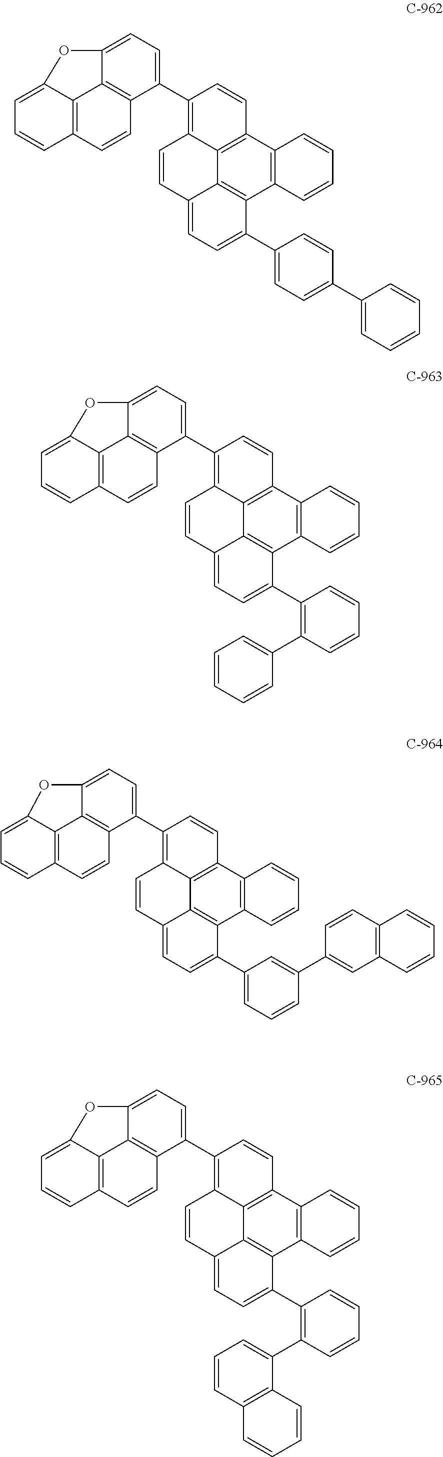 [SMILES: c1ccc(-c2ccc(-c3ccc4ccc5c(-c6ccc7oc8cccc9ccc6c7c98)ccc6c7ccccc7c3c4c56)cc2)cc1 ; c1ccc(-c2ccccc2-c2ccc3ccc4c(-c5ccc6oc7cccc8ccc5c6c87)ccc5c6ccccc6c2c3c45)cc1 ; c1cc(-c2ccc3ccccc3c2)cc(-c2ccc3ccc4c(-c5ccc6oc7cccc8ccc5c6c87)ccc5c6ccccc6c2c3c45)c1 ; c1ccc(-c2ccc3ccc4c(-c5ccc6oc7cccc8ccc5c6c87)ccc5c6ccccc6c2c3c45)c(-c2cccc3ccccc23)c1] 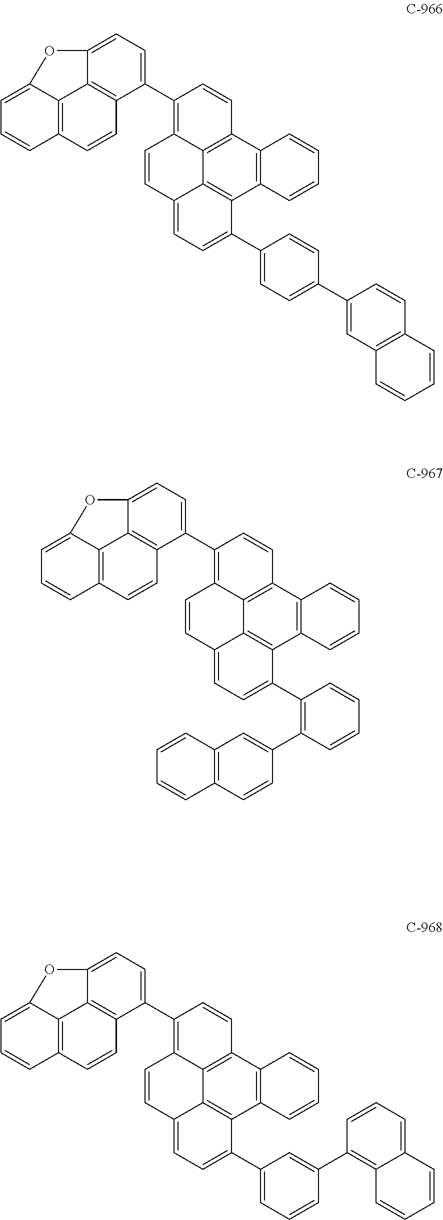 [SMILES: c1ccc2cc(-c3ccc(-c4ccc5ccc6c(-c7ccc8oc9cccc%10ccc7c8c%109)ccc7c8ccccc8c4c5c67)cc3)ccc2c1 ; c1ccc(-c2ccc3ccc4c(-c5ccc6oc7cccc8ccc5c6c87)ccc5c6ccccc6c2c3c45)c(-c2ccc3ccccc3c2)c1 ; c1cc(-c2cccc3ccccc23)cc(-c2ccc3ccc4c(-c5ccc6oc7cccc8ccc5c6c87)ccc5c6ccccc6c2c3c45)c1] 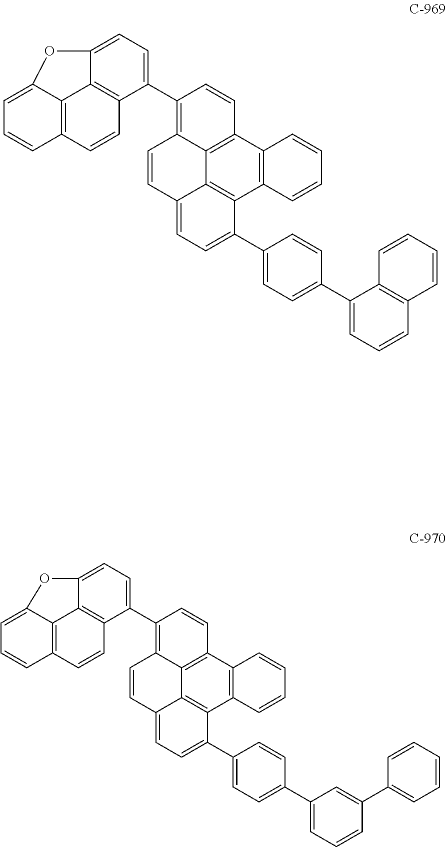 [SMILES: c1ccc2c(-c3ccc(-c4ccc5ccc6c(-c7ccc8oc9cccc%10ccc7c8c%109)ccc7c8ccccc8c4c5c67)cc3)cccc2c1 ; c1ccc(-c2cccc(-c3ccc(-c4ccc5ccc6c(-c7ccc8oc9cccc%10ccc7c8c%109)ccc7c8ccccc8c4c5c67)cc3)c2)cc1] 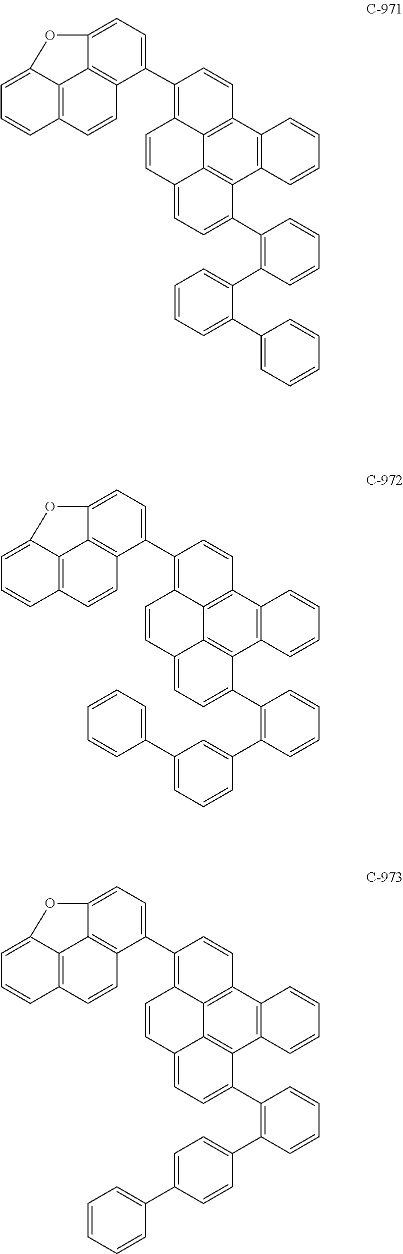 [SMILES: c1ccc(-c2ccccc2-c2ccccc2-c2ccc3ccc4c(-c5ccc6oc7cccc8ccc5c6c87)ccc5c6ccccc6c2c3c45)cc1 ; c1ccc(-c2cccc(-c3ccccc3-c3ccc4ccc5c(-c6ccc7oc8cccc9ccc6c7c98)ccc6c7ccccc7c3c4c56)c2)cc1 ; c1ccc(-c2ccc(-c3ccccc3-c3ccc4ccc5c(-c6ccc7oc8cccc9ccc6c7c98)ccc6c7ccccc7c3c4c56)cc2)cc1] 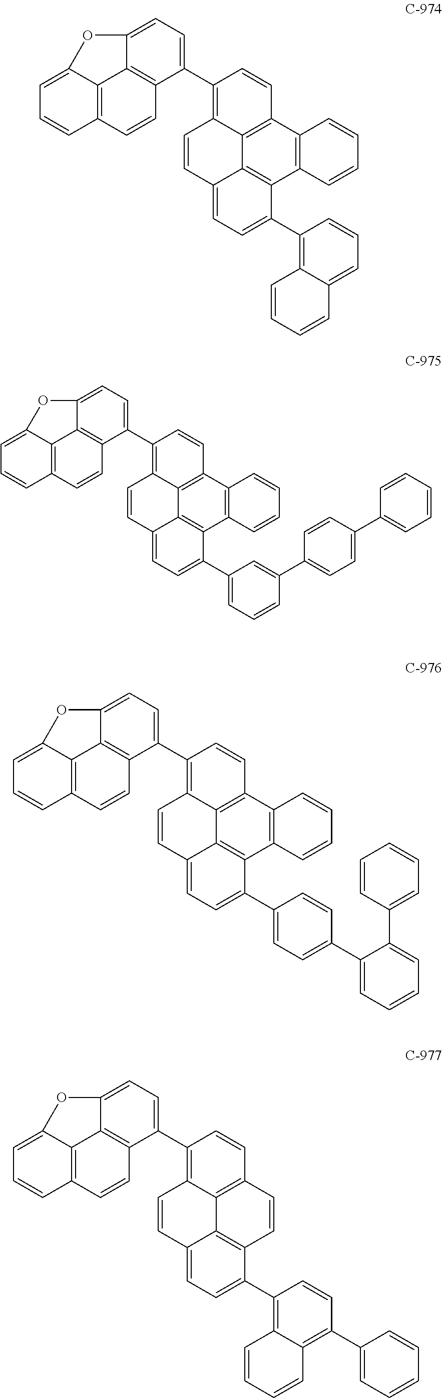 [SMILES: c1ccc2c(-c3ccc4ccc5c(-c6ccc7oc8cccc9ccc6c7c98)ccc6c7ccccc7c3c4c56)cccc2c1 ; c1ccc(-c2ccc(-c3cccc(-c4ccc5ccc6c(-c7ccc8oc9cccc%10ccc7c8c%109)ccc7c8ccccc8c4c5c67)c3)cc2)cc1 ; c1ccc(-c2ccccc2-c2ccc(-c3ccc4ccc5c(-c6ccc7oc8cccc9ccc6c7c98)ccc6c7ccccc7c3c4c56)cc2)cc1 ; c1ccc(-c2ccc(-c3ccc4ccc5c(-c6ccc7oc8cccc9ccc6c7c98)ccc6ccc3c4c65)c3ccccc23)cc1] 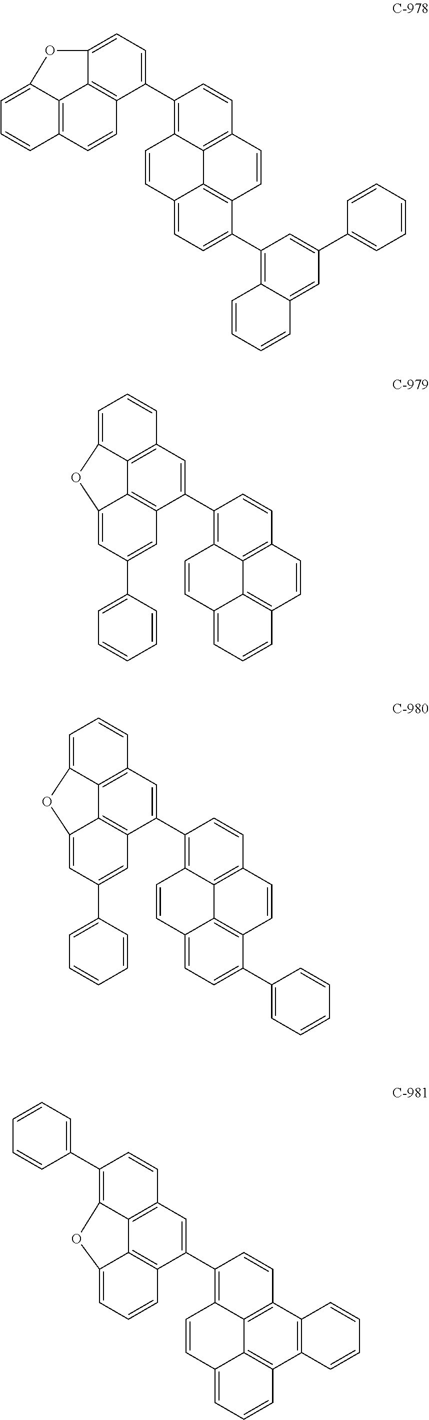 [SMILES: c1ccc(-c2cc(-c3ccc4ccc5c(-c6ccc7oc8cccc9ccc6c7c98)ccc6ccc3c4c65)c3ccccc3c2)cc1 ; c1ccc(-c2cc3oc4cccc5cc(-c6ccc7ccc8cccc9ccc6c7c89)c(c2)c3c54)cc1 ; c1ccc(-c2cc3oc4cccc5cc(-c6ccc7ccc8c(-c9ccccc9)ccc9ccc6c7c98)c(c2)c3c54)cc1 ; c1ccc(-c2ccc3cc(-c4ccc5c6ccccc6c6cccc7ccc4c5c76)c4cccc5oc2c3c54)cc1] 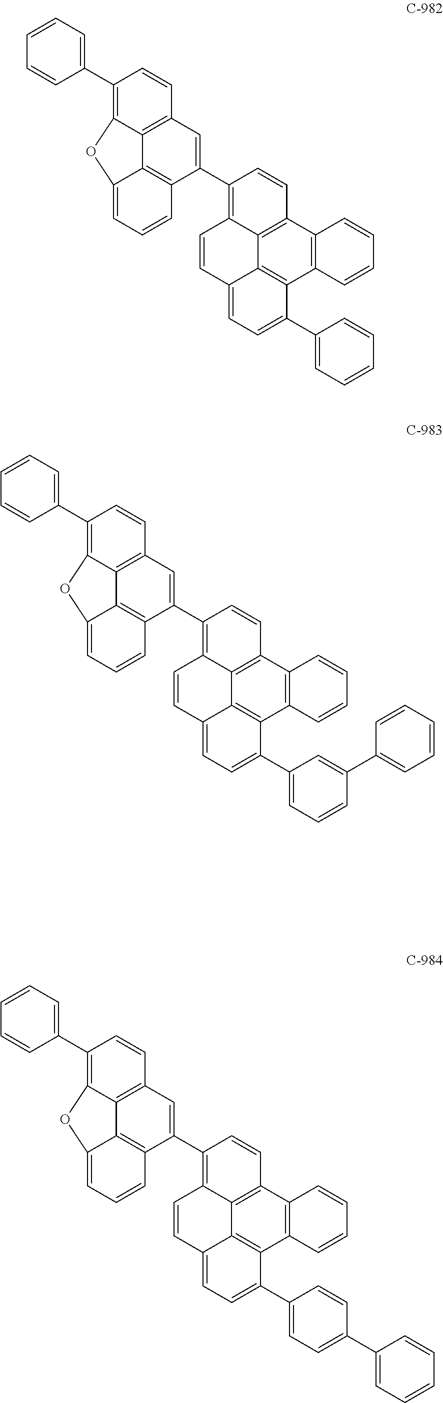 [SMILES: c1ccc(-c2ccc3cc(-c4ccc5c6ccccc6c6c(-c7ccccc7)ccc7ccc4c5c76)c4cccc5oc2c3c54)cc1 ; c1ccc(-c2cccc(-c3ccc4ccc5c(-c6cc7ccc(-c8ccccc8)c8oc9cccc6c9c78)ccc6c7ccccc7c3c4c56)c2)cc1 ; c1ccc(-c2ccc(-c3ccc4ccc5c(-c6cc7ccc(-c8ccccc8)c8oc9cccc6c9c78)ccc6c7ccccc7c3c4c56)cc2)cc1] 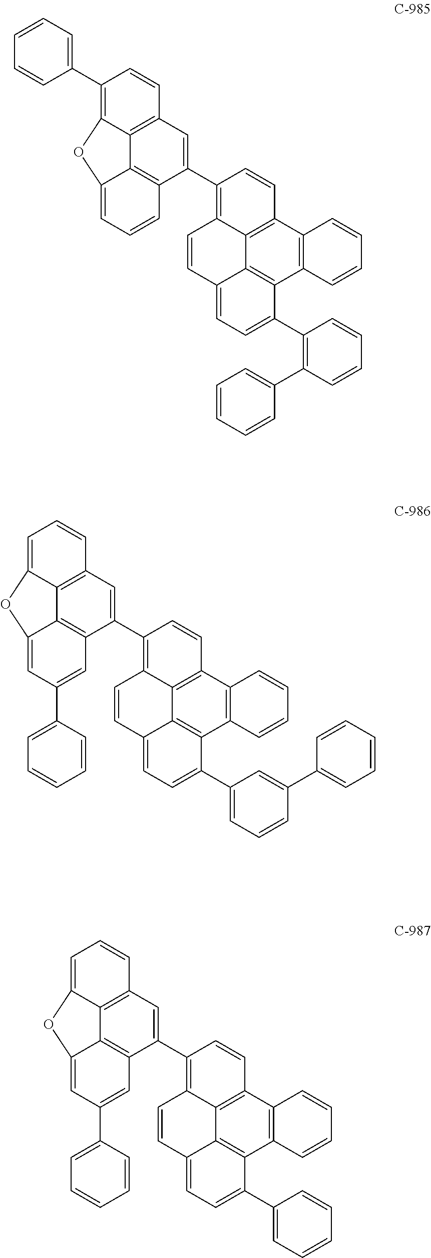 [SMILES: c1ccc(-c2ccccc2-c2ccc3ccc4c(-c5cc6ccc(-c7ccccc7)c7oc8cccc5c8c67)ccc5c6ccccc6c2c3c45)cc1 ; c1ccc(-c2cccc(-c3ccc4ccc5c(-c6cc7cccc8oc9cc(-c%10ccccc%10)cc6c9c78)ccc6c7ccccc7c3c4c56)c2)cc1 ; c1ccc(-c2cc3oc4cccc5cc(-c6ccc7c8ccccc8c8c(-c9ccccc9)ccc9ccc6c7c98)c(c2)c3c54)cc1] 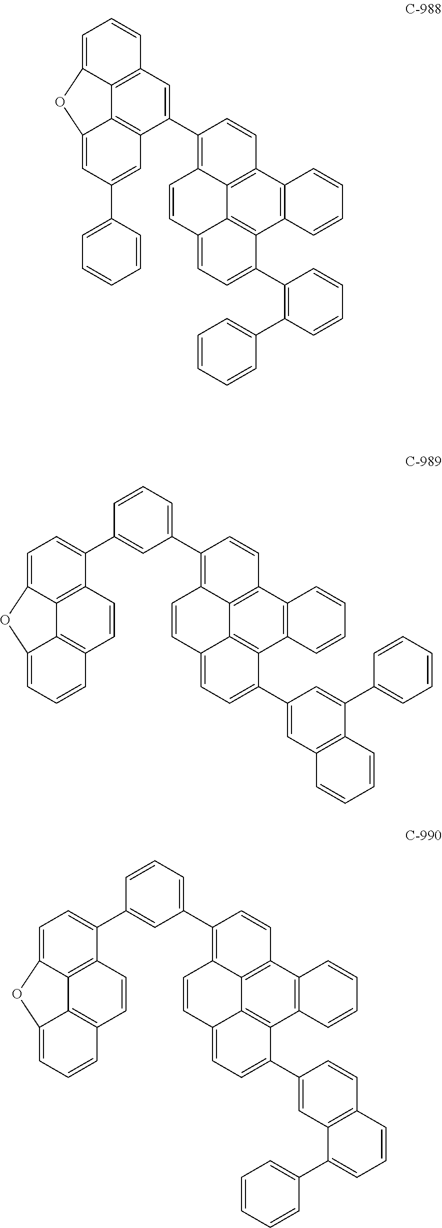 [SMILES: c1ccc(-c2cc3oc4cccc5cc(-c6ccc7c8ccccc8c8c(-c9ccccc9-c9ccccc9)ccc9ccc6c7c98)c(c2)c3c54)cc1 ; c1ccc(-c2cc(-c3ccc4ccc5c(-c6cccc(-c7ccc8oc9cccc%10ccc7c8c%109)c6)ccc6c7ccccc7c3c4c56)cc3ccccc23)cc1 ; c1ccc(-c2cccc3ccc(-c4ccc5ccc6c(-c7cccc(-c8ccc9oc%10cccc%11ccc8c9c%11%10)c7)ccc7c8ccccc8c4c5c67)cc23)cc1] 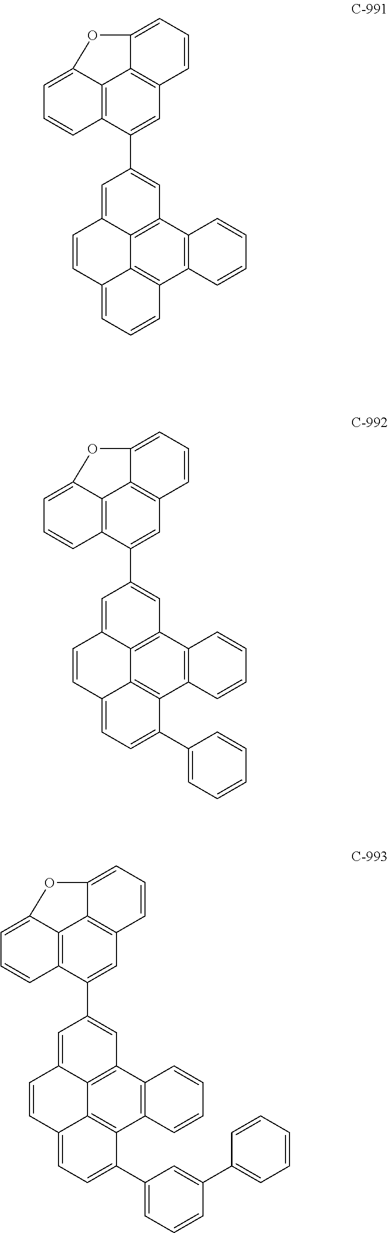 [SMILES: c1cc2cc(-c3cc4ccc5cccc6c7ccccc7c(c3)c4c56)c3cccc4oc(c1)c2c43 ; c1ccc(-c2ccc3ccc4cc(-c5cc6cccc7oc8cccc5c8c67)cc5c6ccccc6c2c3c45)cc1 ; c1ccc(-c2cccc(-c3ccc4ccc5cc(-c6cc7cccc8oc9cccc6c9c78)cc6c7ccccc7c3c4c56)c2)cc1] 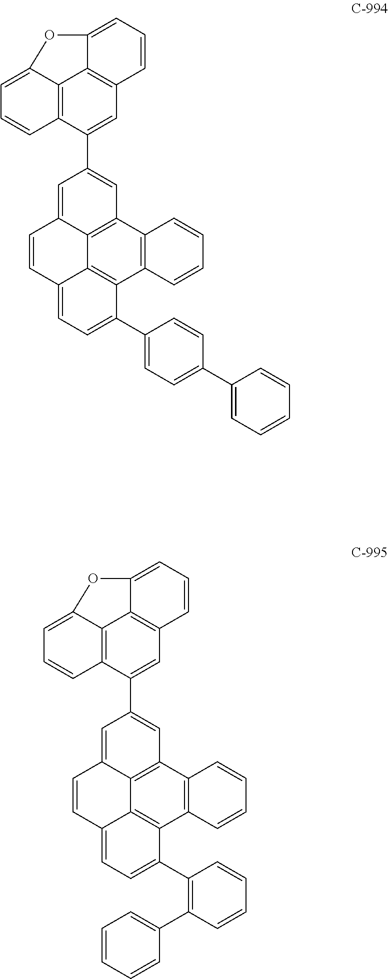 [SMILES: c1ccc(-c2ccc(-c3ccc4ccc5cc(-c6cc7cccc8oc9cccc6c9c78)cc6c7ccccc7c3c4c56)cc2)cc1 ; c1ccc(-c2ccccc2-c2ccc3ccc4cc(-c5cc6cccc7oc8cccc5c8c67)cc5c6ccccc6c2c3c45)cc1] 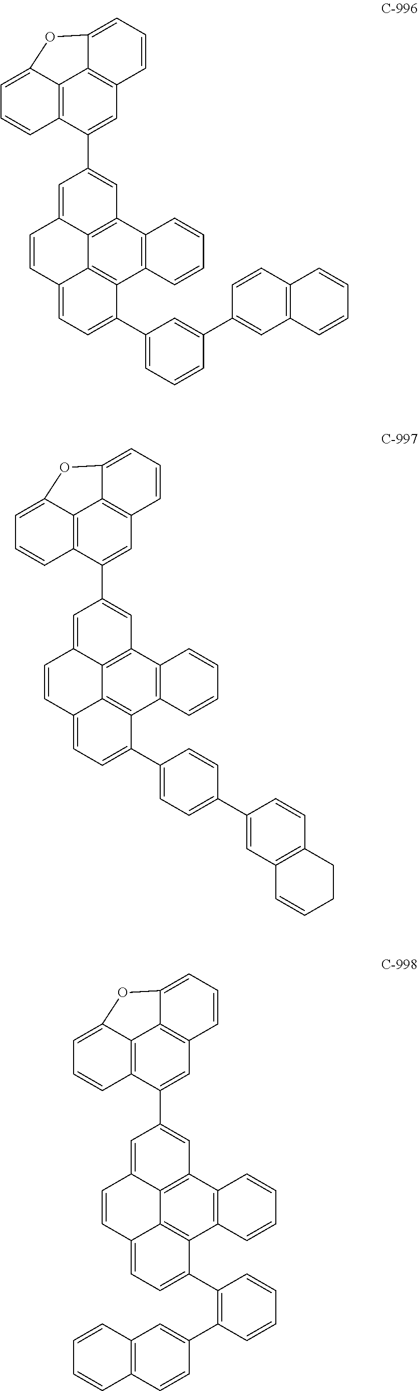 [SMILES: c1cc(-c2ccc3ccccc3c2)cc(-c2ccc3ccc4cc(-c5cc6cccc7oc8cccc5c8c67)cc5c6ccccc6c2c3c45)c1 ; C1=Cc2cc(-c3ccc(-c4ccc5ccc6cc(-c7cc8cccc9oc%10cccc7c%10c89)cc7c8ccccc8c4c5c67)cc3)ccc2CC1 ; c1ccc(-c2ccc3ccc4cc(-c5cc6cccc7oc8cccc5c8c67)cc5c6ccccc6c2c3c45)c(-c2ccc3ccccc3c2)c1] 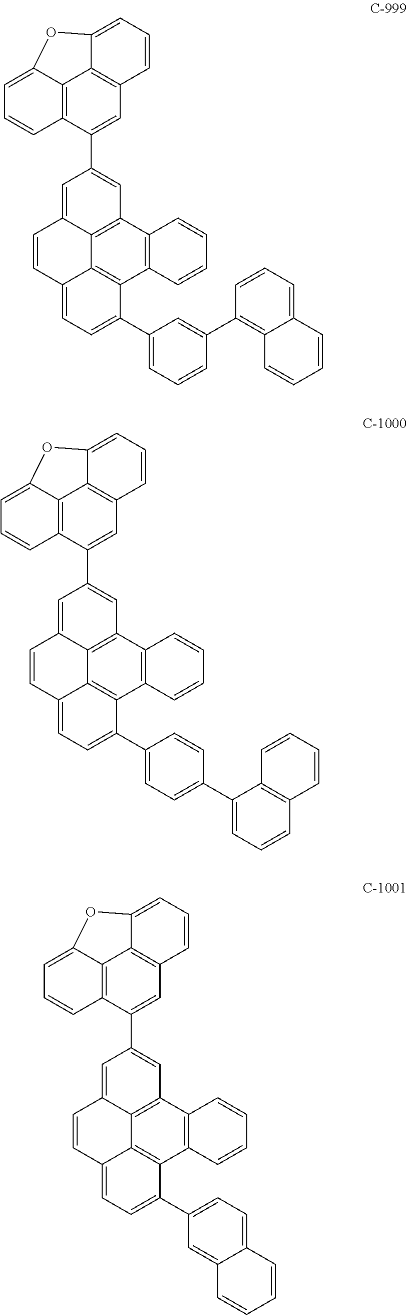 [SMILES: c1cc(-c2cccc3ccccc23)cc(-c2ccc3ccc4cc(-c5cc6cccc7oc8cccc5c8c67)cc5c6ccccc6c2c3c45)c1 ; c1ccc2c(-c3ccc(-c4ccc5ccc6cc(-c7cc8cccc9oc%10cccc7c%10c89)cc7c8ccccc8c4c5c67)cc3)cccc2c1 ; c1ccc2cc(-c3ccc4ccc5cc(-c6cc7cccc8oc9cccc6c9c78)cc6c7ccccc7c3c4c56)ccc2c1] 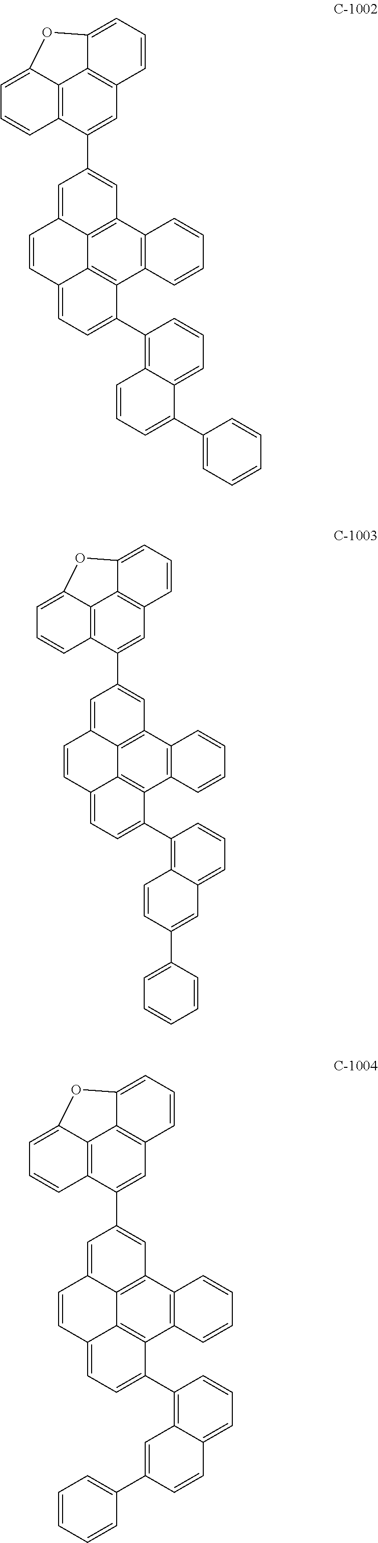 [SMILES: c1ccc(-c2cccc3c(-c4ccc5ccc6cc(-c7cc8cccc9oc%10cccc7c%10c89)cc7c8ccccc8c4c5c67)cccc23)cc1 ; c1ccc(-c2ccc3c(-c4ccc5ccc6cc(-c7cc8cccc9oc%10cccc7c%10c89)cc7c8ccccc8c4c5c67)cccc3c2)cc1 ; c1ccc(-c2ccc3cccc(-c4ccc5ccc6cc(-c7cc8cccc9oc%10cccc7c%10c89)cc7c8ccccc8c4c5c67)c3c2)cc1] 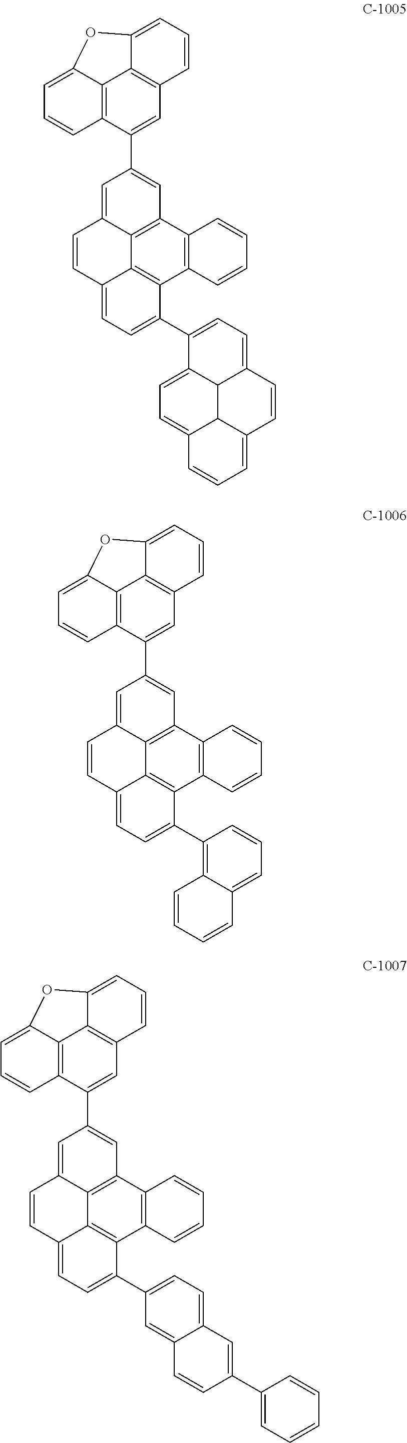 [SMILES: C1=CC2=CC=C3C(c4ccc5ccc6cc(-c7cc8cccc9oc%10cccc7c%10c89)cc7c8ccccc8c4c5c67)=CC=C4C=CC(=C1)C2C43 ; c1ccc2c(-c3ccc4ccc5cc(-c6cc7cccc8oc9cccc6c9c78)cc6c7ccccc7c3c4c56)cccc2c1 ; c1ccc(-c2ccc3cc(-c4ccc5ccc6cc(-c7cc8cccc9oc%10cccc7c%10c89)cc7c8ccccc8c4c5c67)ccc3c2)cc1] 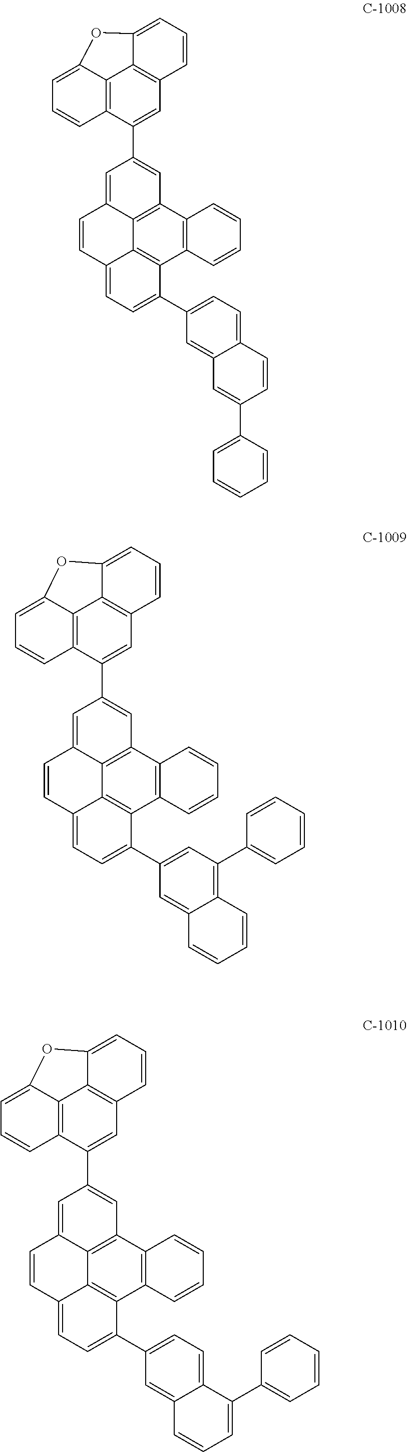 [SMILES: c1ccc(-c2ccc3ccc(-c4ccc5ccc6cc(-c7cc8cccc9oc%10cccc7c%10c89)cc7c8ccccc8c4c5c67)cc3c2)cc1 ; c1ccc(-c2cc(-c3ccc4ccc5cc(-c6cc7cccc8oc9cccc6c9c78)cc6c7ccccc7c3c4c56)cc3ccccc23)cc1 ; c1ccc(-c2cccc3cc(-c4ccc5ccc6cc(-c7cc8cccc9oc%10cccc7c%10c89)cc7c8ccccc8c4c5c67)ccc23)cc1] 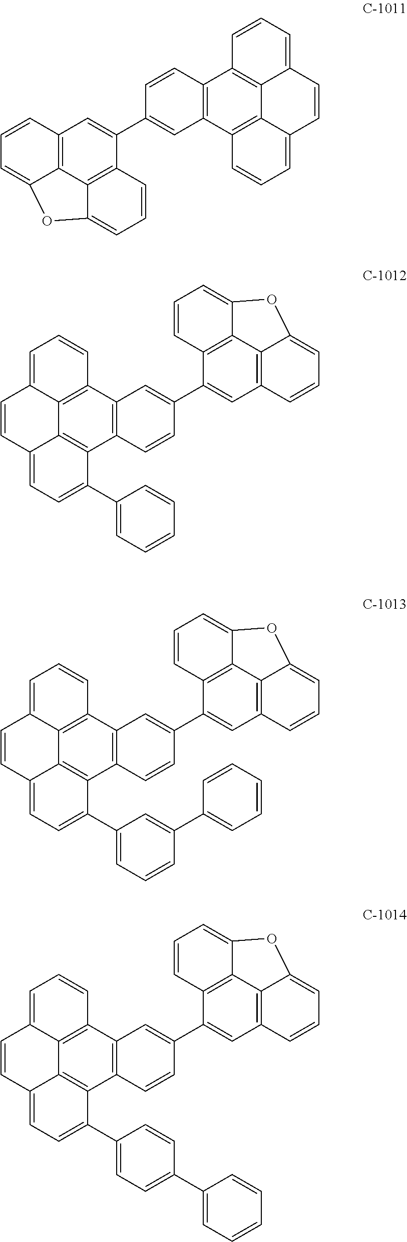 [SMILES: c1cc2cc(-c3ccc4c(c3)c3cccc5ccc6cccc4c6c53)c3cccc4oc(c1)c2c43 ; c1ccc(-c2ccc3ccc4cccc5c6cc(-c7cc8cccc9oc%10cccc7c%10c89)ccc6c2c3c45)cc1 ; c1ccc(-c2cccc(-c3ccc4ccc5cccc6c7cc(-c8cc9cccc%10oc%11cccc8c%11c9%10)ccc7c3c4c56)c2)cc1 ; c1ccc(-c2ccc(-c3ccc4ccc5cccc6c7cc(-c8cc9cccc%10oc%11cccc8c%11c9%10)ccc7c3c4c56)cc2)cc1] 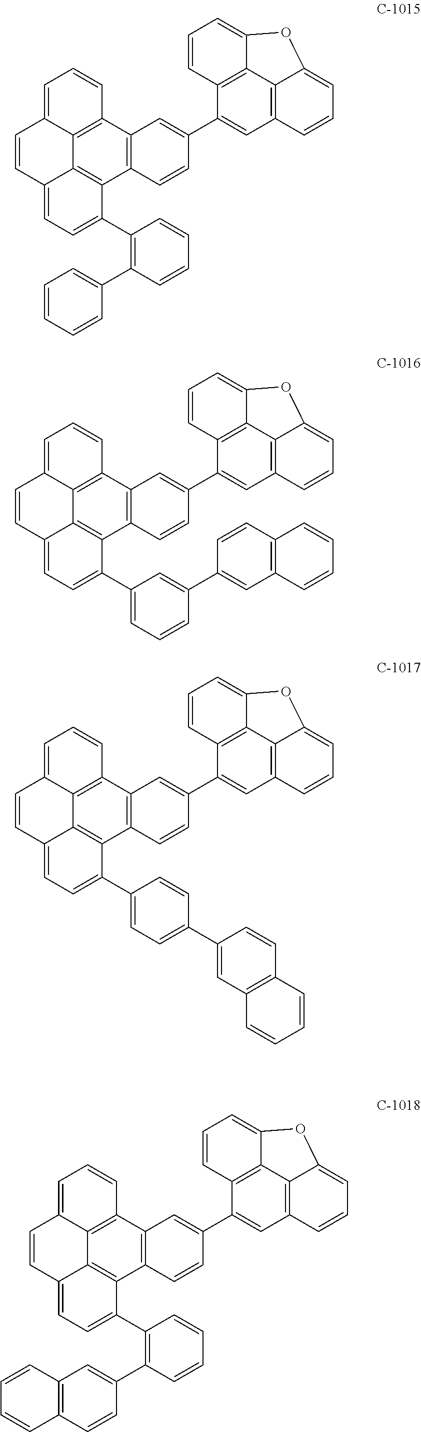 [SMILES: c1ccc(-c2ccccc2-c2ccc3ccc4cccc5c6cc(-c7cc8cccc9oc%10cccc7c%10c89)ccc6c2c3c45)cc1 ; c1cc(-c2ccc3ccccc3c2)cc(-c2ccc3ccc4cccc5c6cc(-c7cc8cccc9oc%10cccc7c%10c89)ccc6c2c3c45)c1 ; c1ccc2cc(-c3ccc(-c4ccc5ccc6cccc7c8cc(-c9cc%10cccc%11oc%12cccc9c%12c%10%11)ccc8c4c5c67)cc3)ccc2c1 ; c1ccc(-c2ccc3ccc4cccc5c6cc(-c7cc8cccc9oc%10cccc7c%10c89)ccc6c2c3c45)c(-c2ccc3ccccc3c2)c1] 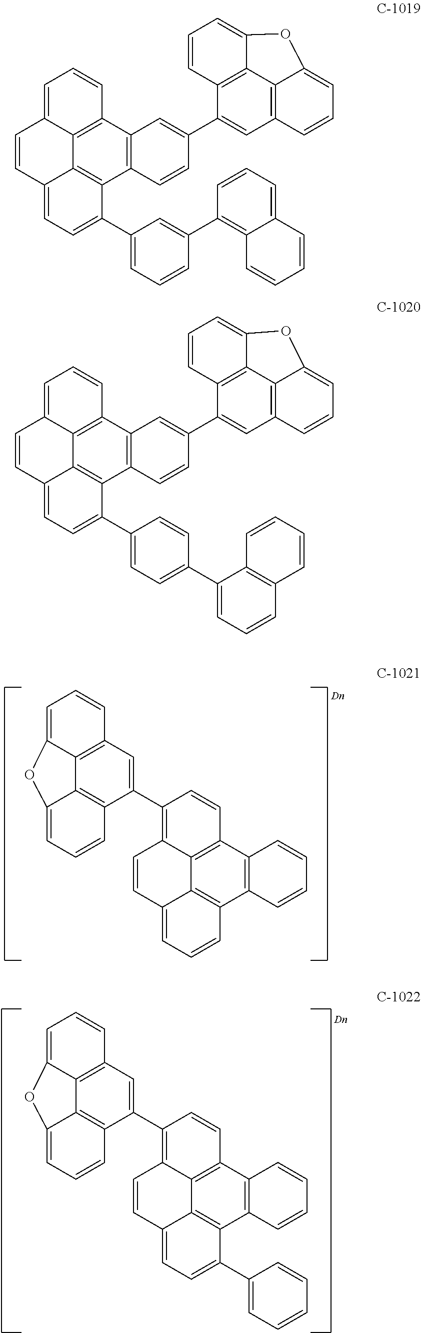 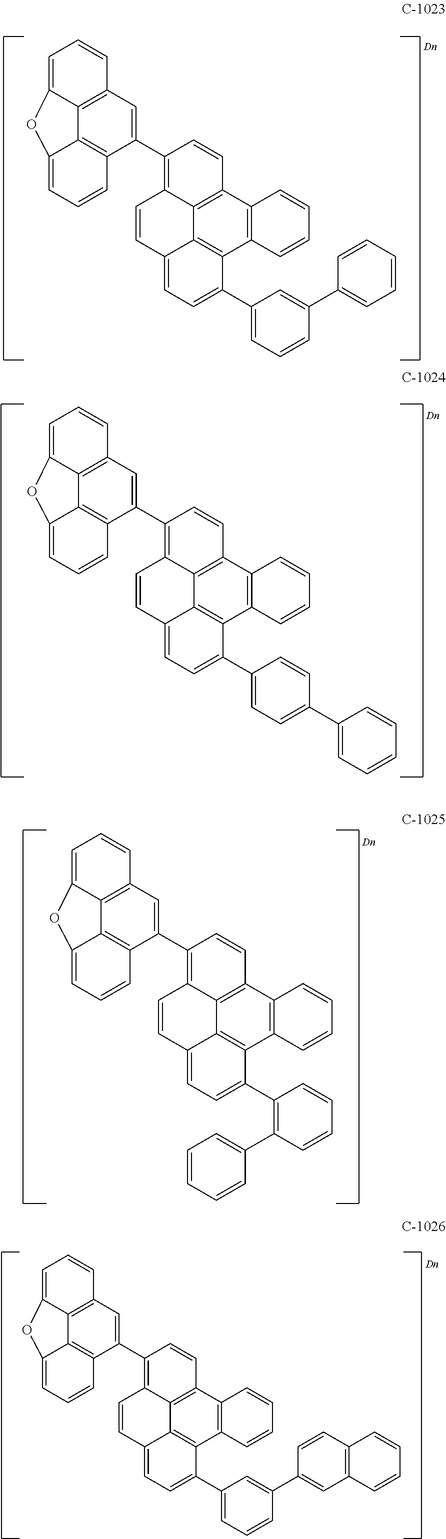 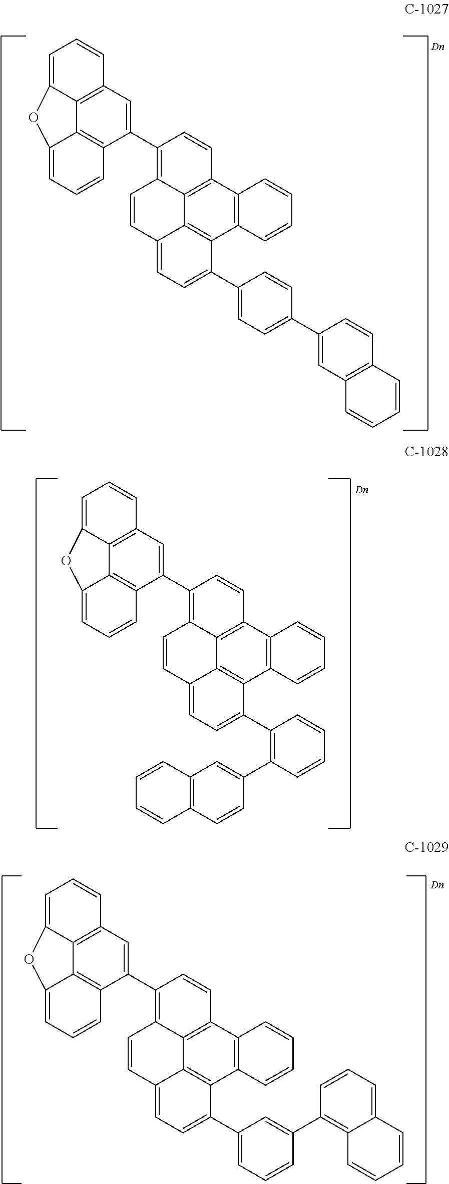 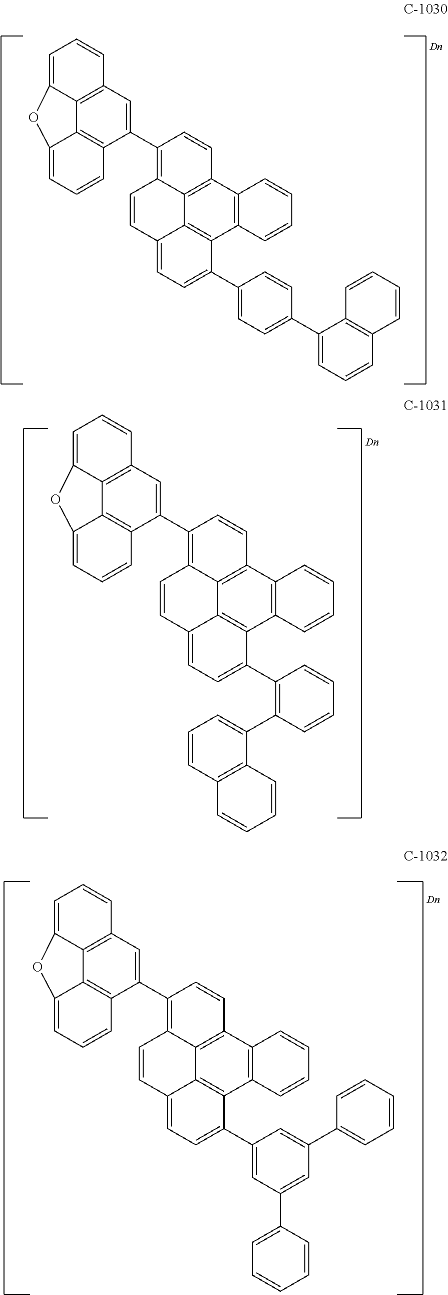 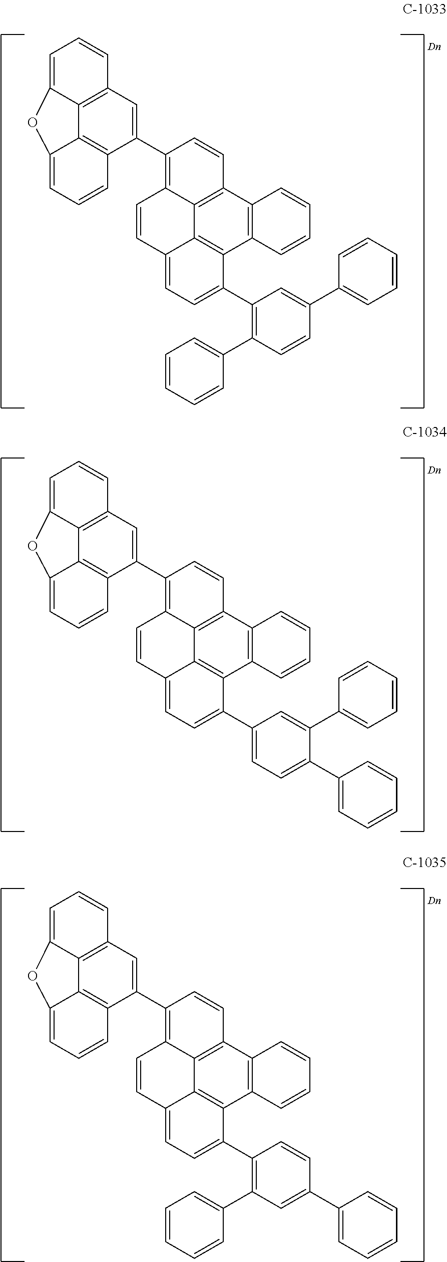 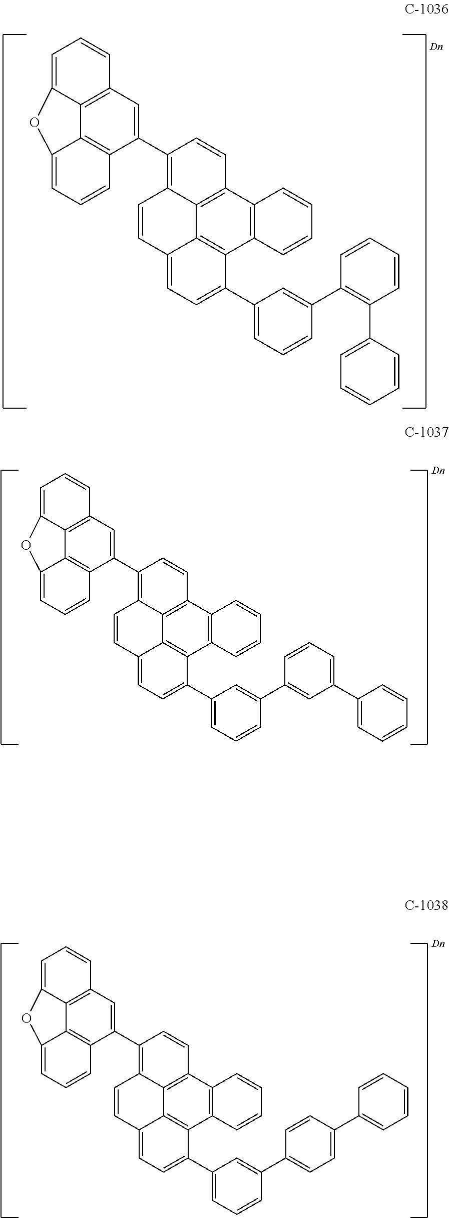 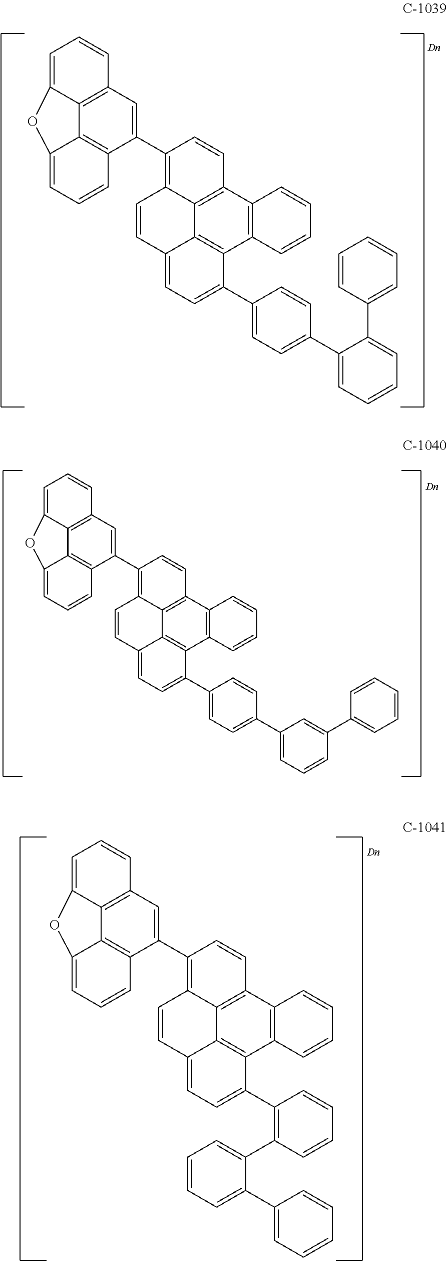 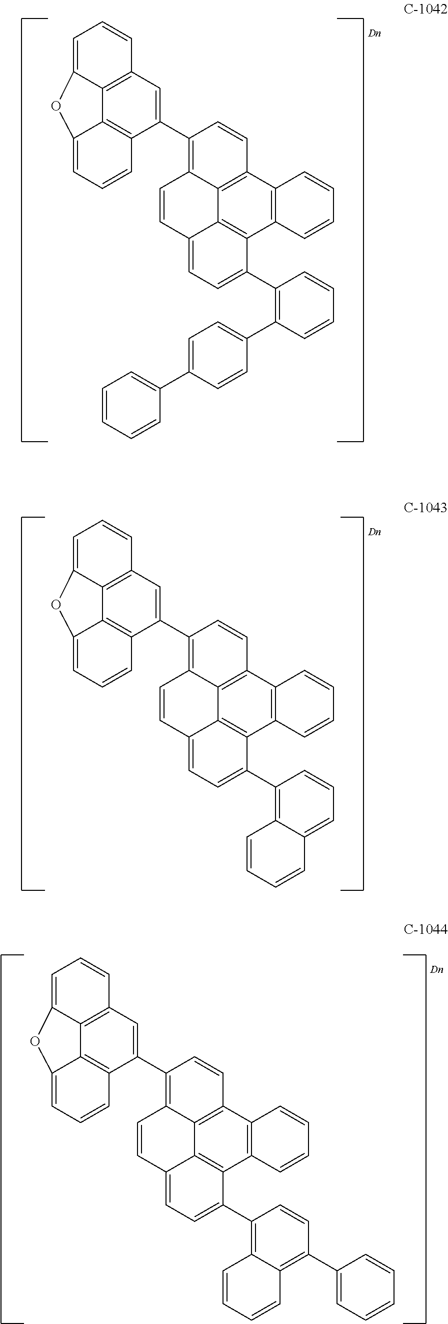 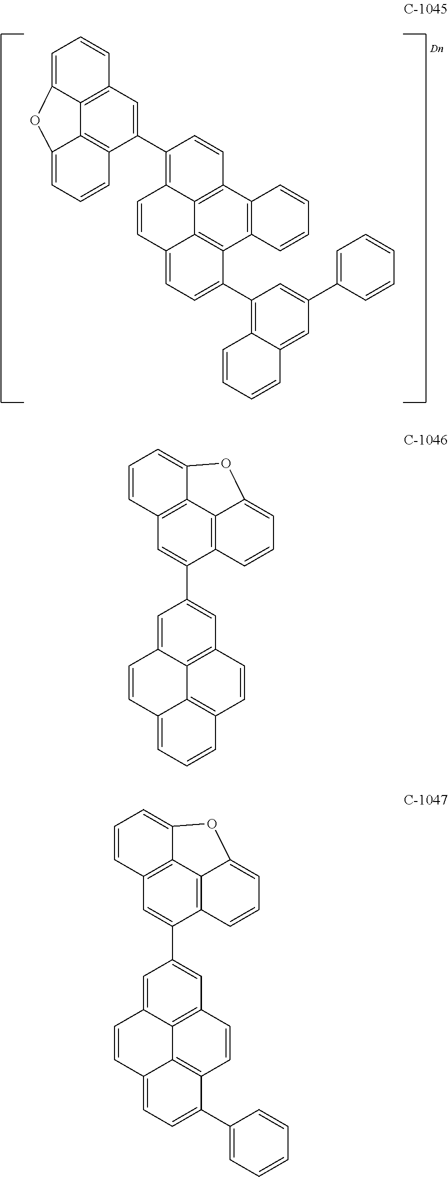 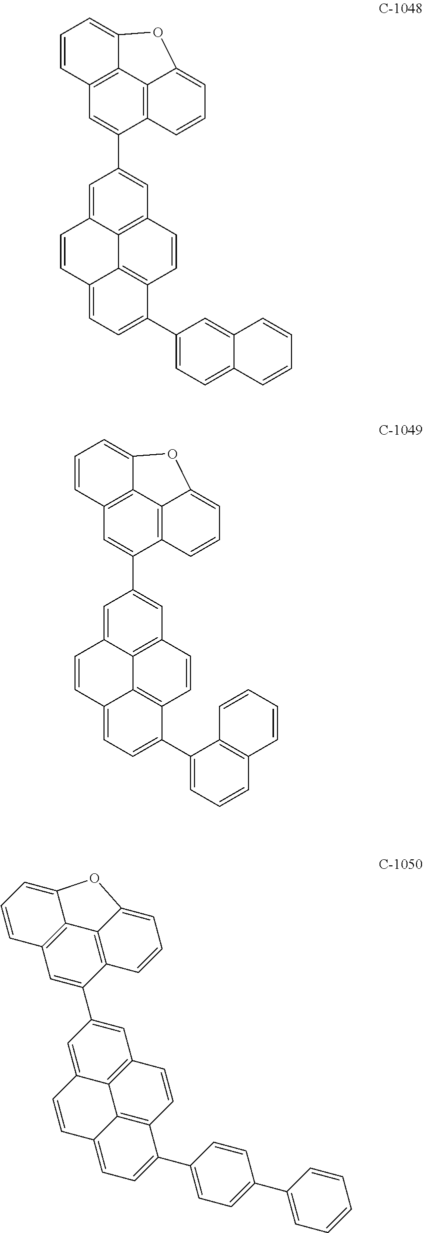 [SMILES: c1ccc2cc(-c3ccc4ccc5cc(-c6cc7cccc8oc9cccc6c9c78)cc6ccc3c4c56)ccc2c1 ; c1ccc2c(-c3ccc4ccc5cc(-c6cc7cccc8oc9cccc6c9c78)cc6ccc3c4c56)cccc2c1 ; c1ccc(-c2ccc(-c3ccc4ccc5cc(-c6cc7cccc8oc9cccc6c9c78)cc6ccc3c4c56)cc2)cc1] 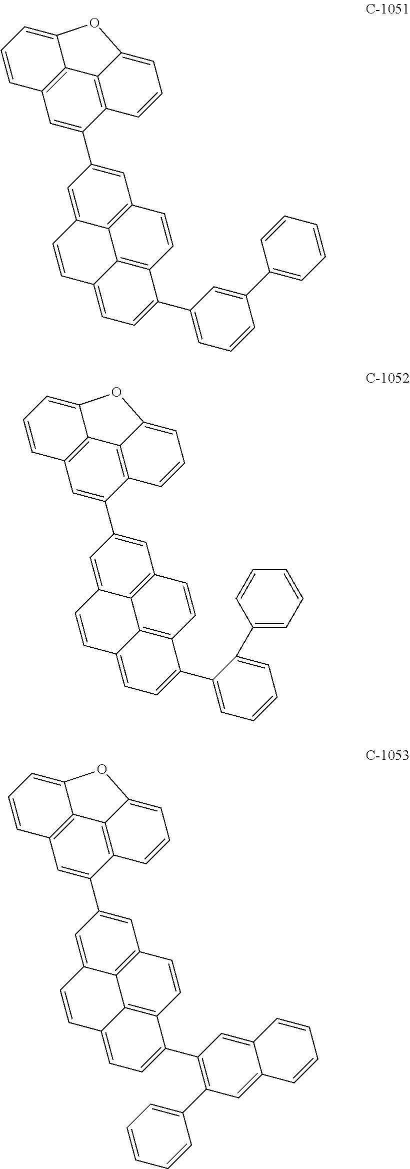 [SMILES: c1ccc(-c2cccc(-c3ccc4ccc5cc(-c6cc7cccc8oc9cccc6c9c78)cc6ccc3c4c56)c2)cc1 ; c1ccc(-c2ccccc2-c2ccc3ccc4cc(-c5cc6cccc7oc8cccc5c8c67)cc5ccc2c3c45)cc1 ; c1ccc(-c2cc3ccccc3cc2-c2ccc3ccc4cc(-c5cc6cccc7oc8cccc5c8c67)cc5ccc2c3c45)cc1] 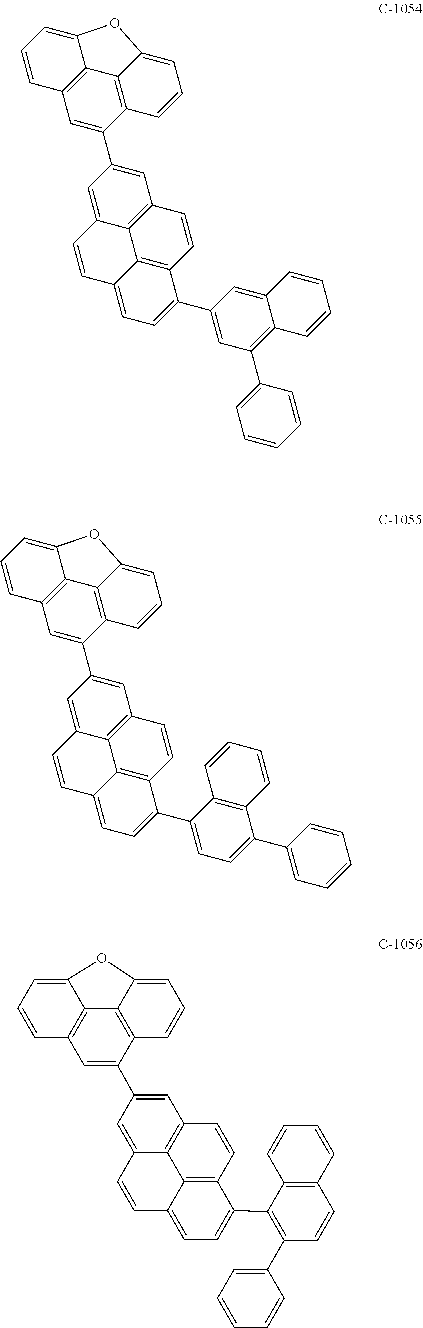 [SMILES: c1ccc(-c2cc(-c3ccc4ccc5cc(-c6cc7cccc8oc9cccc6c9c78)cc6ccc3c4c56)cc3ccccc23)cc1 ; c1ccc(-c2ccc(-c3ccc4ccc5cc(-c6cc7cccc8oc9cccc6c9c78)cc6ccc3c4c56)c3ccccc23)cc1 ; c1ccc(-c2ccc3ccccc3c2-c2ccc3ccc4cc(-c5cc6cccc7oc8cccc5c8c67)cc5ccc2c3c45)cc1] 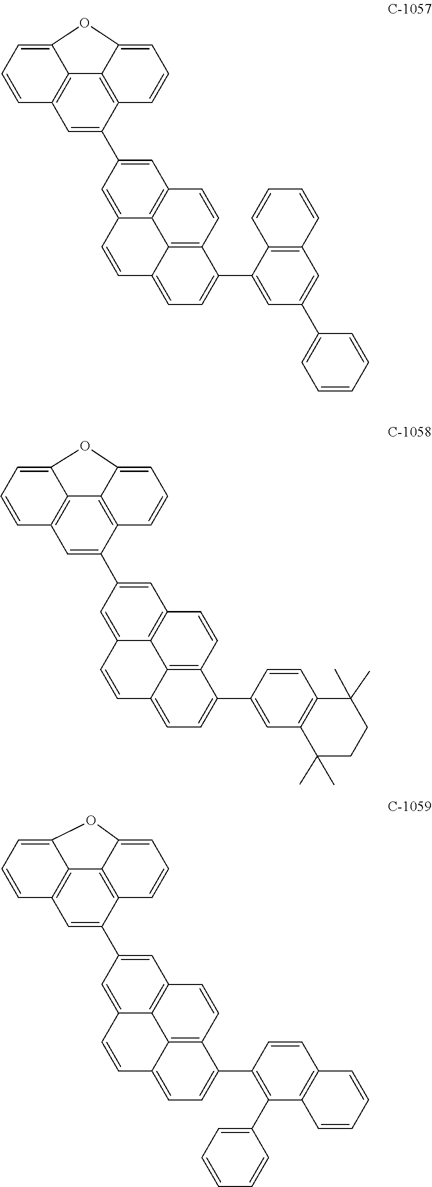 [SMILES: c1ccc(-c2cc(-c3ccc4ccc5cc(-c6cc7cccc8oc9cccc6c9c78)cc6ccc3c4c56)c3ccccc3c2)cc1 ; CC1(C)CCC(C)(C)c2cc(-c3ccc4ccc5cc(-c6cc7cccc8oc9cccc6c9c78)cc6ccc3c4c56)ccc21 ; c1ccc(-c2c(-c3ccc4ccc5cc(-c6cc7cccc8oc9cccc6c9c78)cc6ccc3c4c56)ccc3ccccc23)cc1] 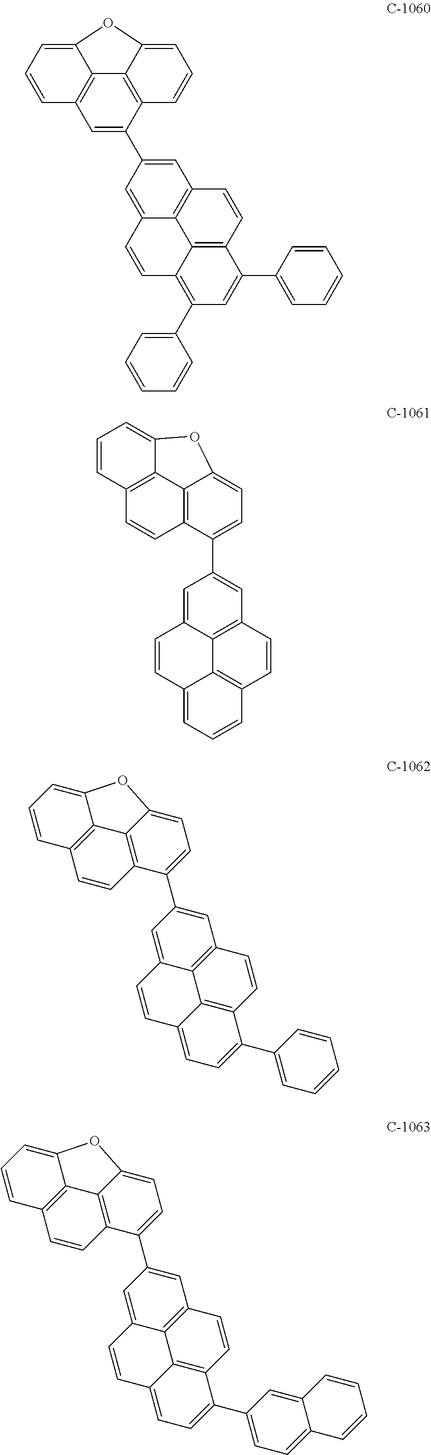 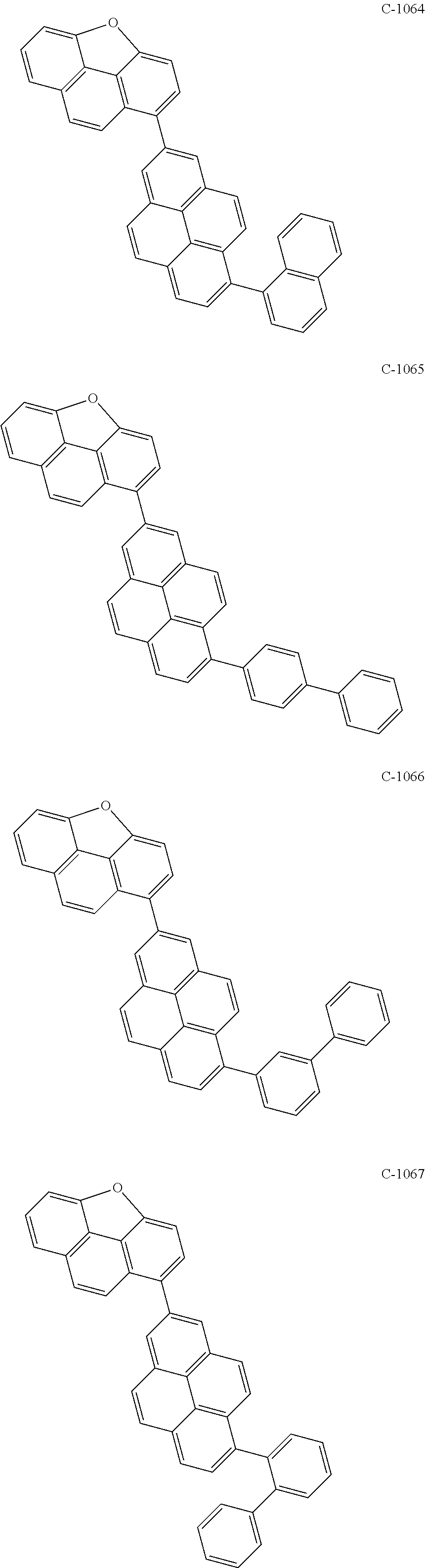 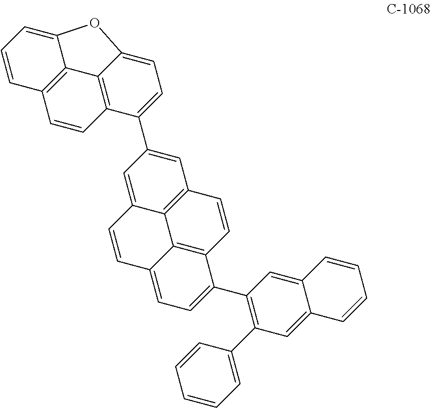 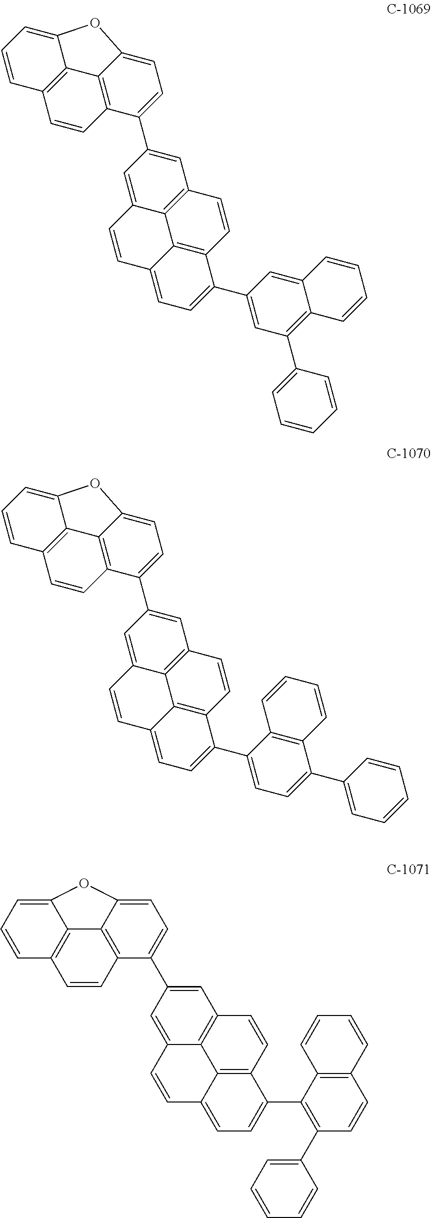 [SMILES: c1ccc(-c2cc(-c3ccc4ccc5cc(-c6ccc7oc8cccc9ccc6c7c98)cc6ccc3c4c56)cc3ccccc23)cc1 ; c1ccc(-c2ccc(-c3ccc4ccc5cc(-c6ccc7oc8cccc9ccc6c7c98)cc6ccc3c4c56)c3ccccc23)cc1 ; c1ccc(-c2ccc3ccccc3c2-c2ccc3ccc4cc(-c5ccc6oc7cccc8ccc5c6c87)cc5ccc2c3c45)cc1] 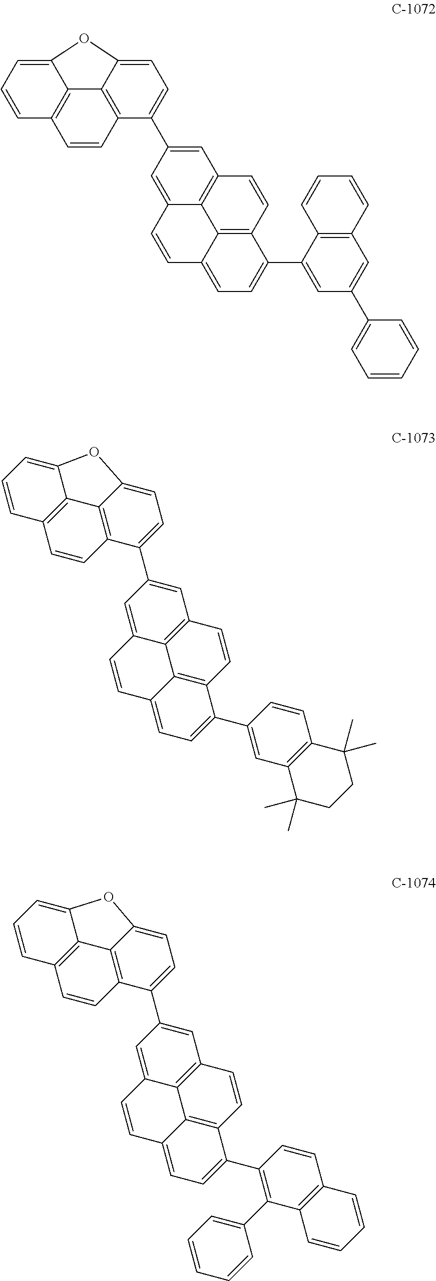 [SMILES: c1ccc(-c2cc(-c3ccc4ccc5cc(-c6ccc7oc8cccc9ccc6c7c98)cc6ccc3c4c56)c3ccccc3c2)cc1 ; CC1(C)CCC(C)(C)c2cc(-c3ccc4ccc5cc(-c6ccc7oc8cccc9ccc6c7c98)cc6ccc3c4c56)ccc21 ; c1ccc(-c2c(-c3ccc4ccc5cc(-c6ccc7oc8cccc9ccc6c7c98)cc6ccc3c4c56)ccc3ccccc23)cc1] 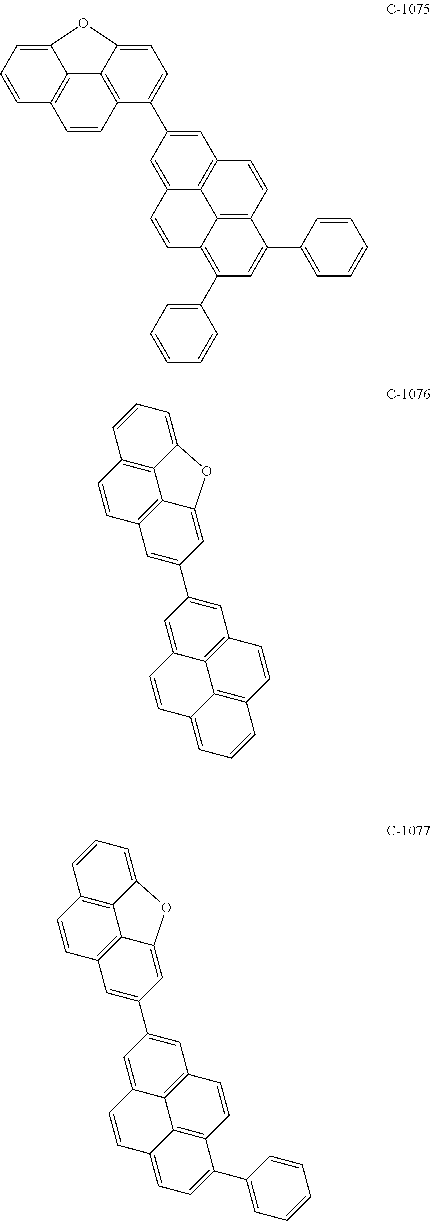 [SMILES: c1ccc(-c2cc(-c3ccccc3)c3ccc4cc(-c5ccc6oc7cccc8ccc5c6c87)cc5ccc2c3c54)cc1 ; c1cc2ccc3cc(-c4cc5ccc6cccc7oc(c4)c5c67)cc4ccc(c1)c2c34 ; c1ccc(-c2ccc3ccc4cc(-c5cc6ccc7cccc8oc(c5)c6c78)cc5ccc2c3c45)cc1] 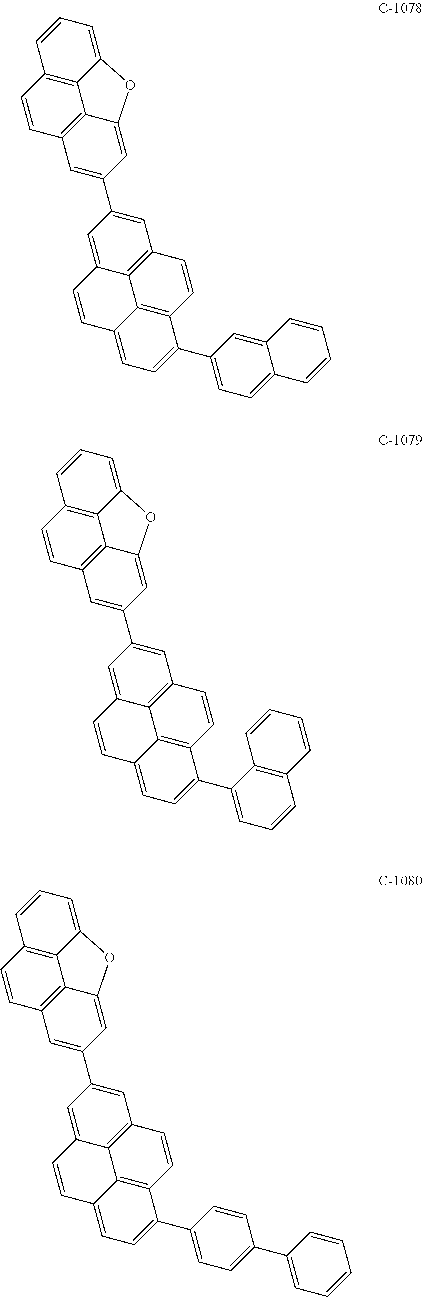 [SMILES: c1ccc2cc(-c3ccc4ccc5cc(-c6cc7ccc8cccc9oc(c6)c7c89)cc6ccc3c4c56)ccc2c1 ; c1ccc2c(-c3ccc4ccc5cc(-c6cc7ccc8cccc9oc(c6)c7c89)cc6ccc3c4c56)cccc2c1 ; c1ccc(-c2ccc(-c3ccc4ccc5cc(-c6cc7ccc8cccc9oc(c6)c7c89)cc6ccc3c4c56)cc2)cc1] 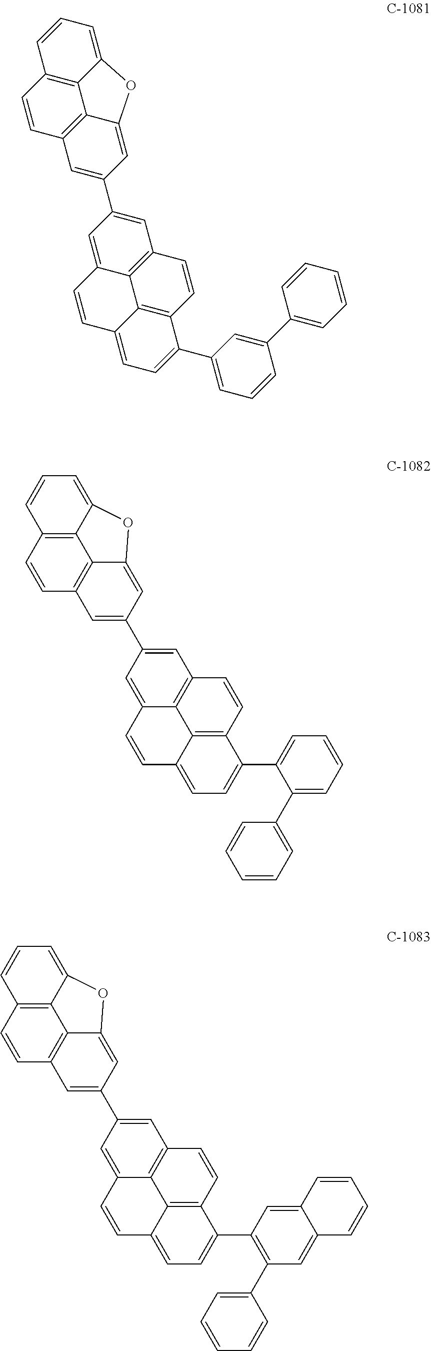 [SMILES: c1ccc(-c2cccc(-c3ccc4ccc5cc(-c6cc7ccc8cccc9oc(c6)c7c89)cc6ccc3c4c56)c2)cc1 ; c1ccc(-c2ccccc2-c2ccc3ccc4cc(-c5cc6ccc7cccc8oc(c5)c6c78)cc5ccc2c3c45)cc1 ; c1ccc(-c2cc3ccccc3cc2-c2ccc3ccc4cc(-c5cc6ccc7cccc8oc(c5)c6c78)cc5ccc2c3c45)cc1] 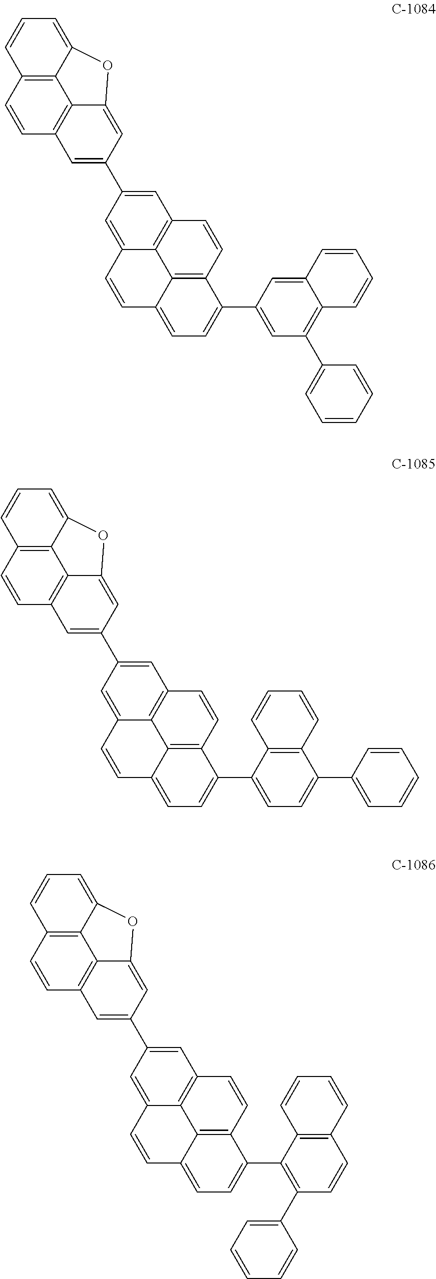 [SMILES: c1ccc(-c2cc(-c3ccc4ccc5cc(-c6cc7ccc8cccc9oc(c6)c7c89)cc6ccc3c4c56)cc3ccccc23)cc1 ; c1ccc(-c2ccc(-c3ccc4ccc5cc(-c6cc7ccc8cccc9oc(c6)c7c89)cc6ccc3c4c56)c3ccccc23)cc1 ; c1ccc(-c2ccc3ccccc3c2-c2ccc3ccc4cc(-c5cc6ccc7cccc8oc(c5)c6c78)cc5ccc2c3c45)cc1] 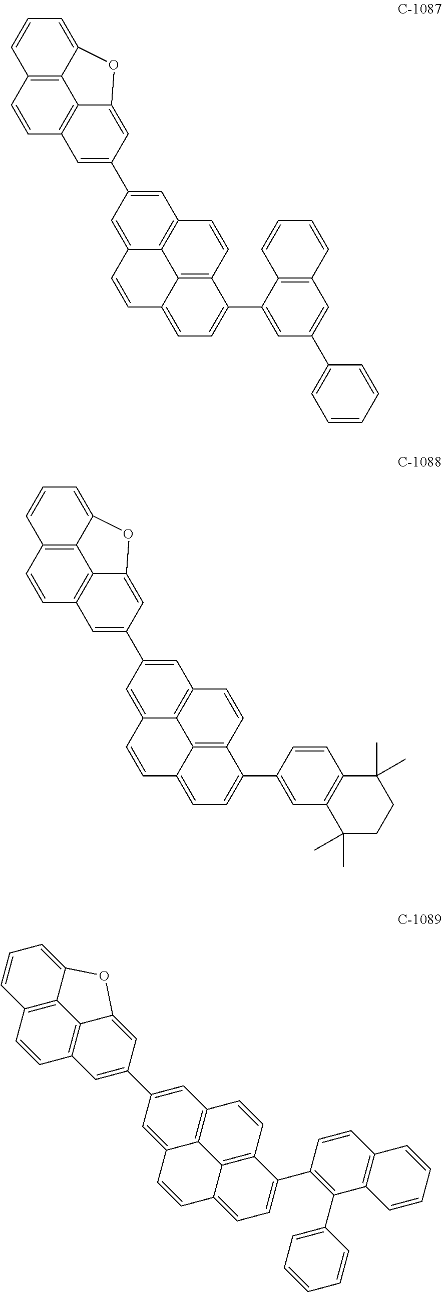 [SMILES: c1ccc(-c2cc(-c3ccc4ccc5cc(-c6cc7ccc8cccc9oc(c6)c7c89)cc6ccc3c4c56)c3ccccc3c2)cc1 ; CC1(C)CCC(C)(C)c2cc(-c3ccc4ccc5cc(-c6cc7ccc8cccc9oc(c6)c7c89)cc6ccc3c4c56)ccc21 ; c1ccc(-c2c(-c3ccc4ccc5cc(-c6cc7ccc8cccc9oc(c6)c7c89)cc6ccc3c4c56)ccc3ccccc23)cc1] 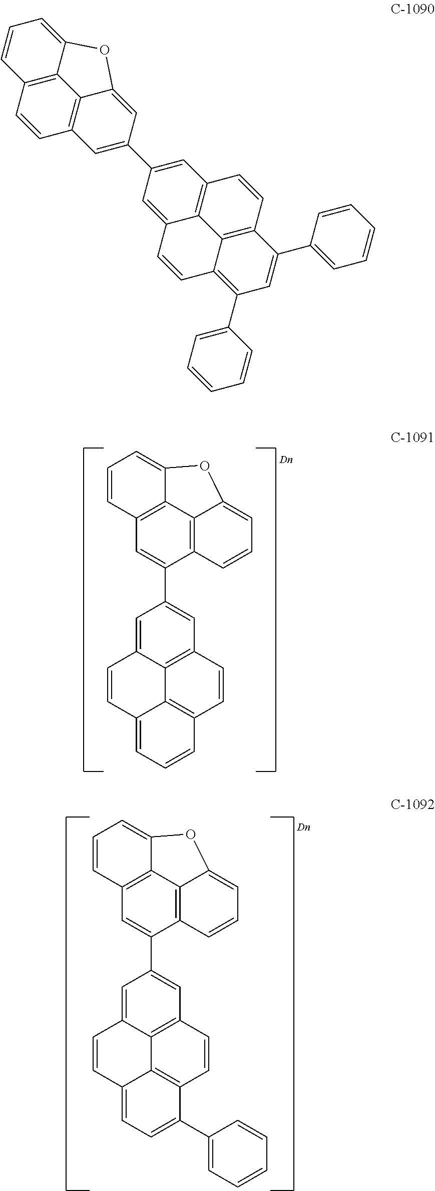 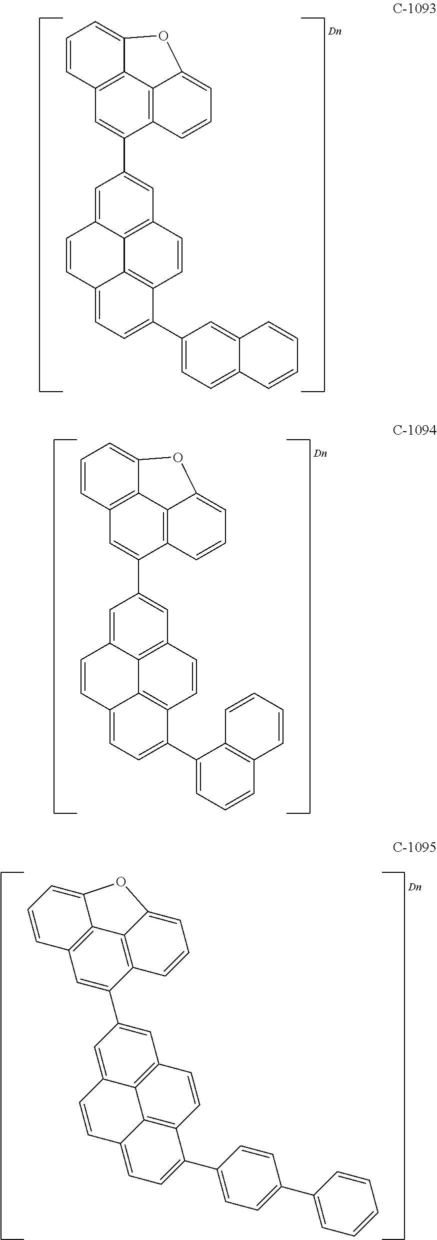 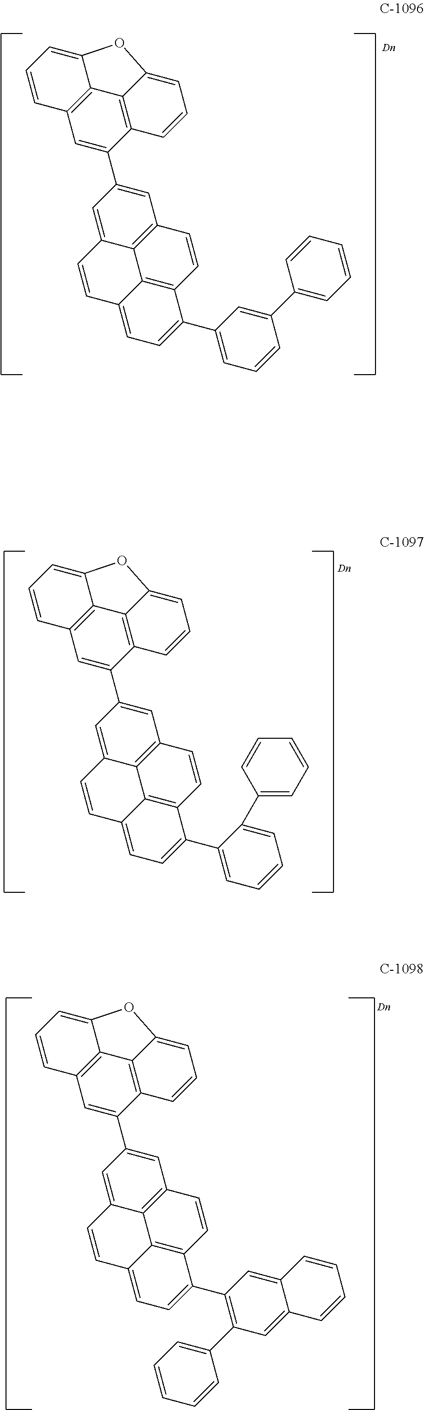 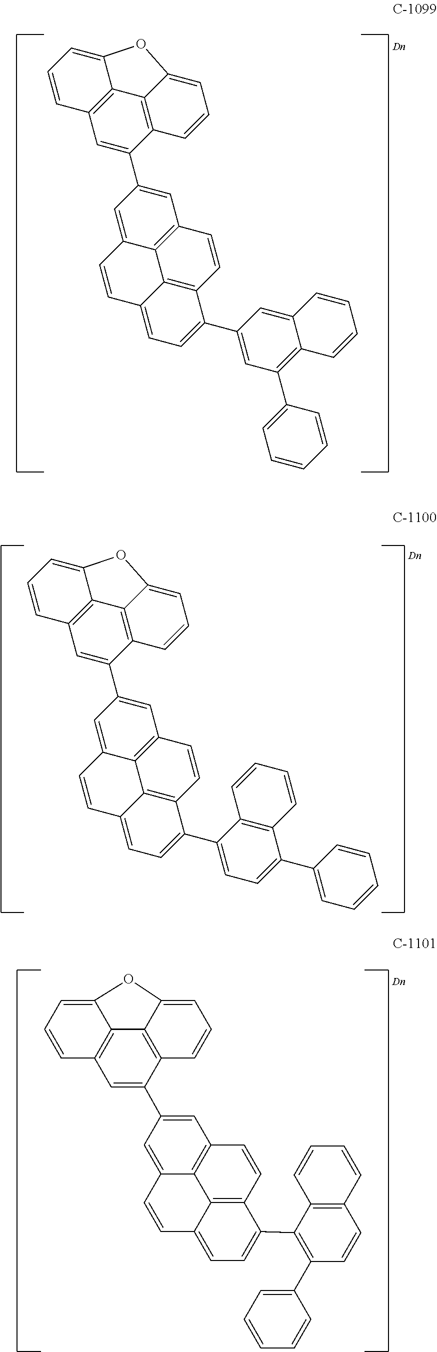 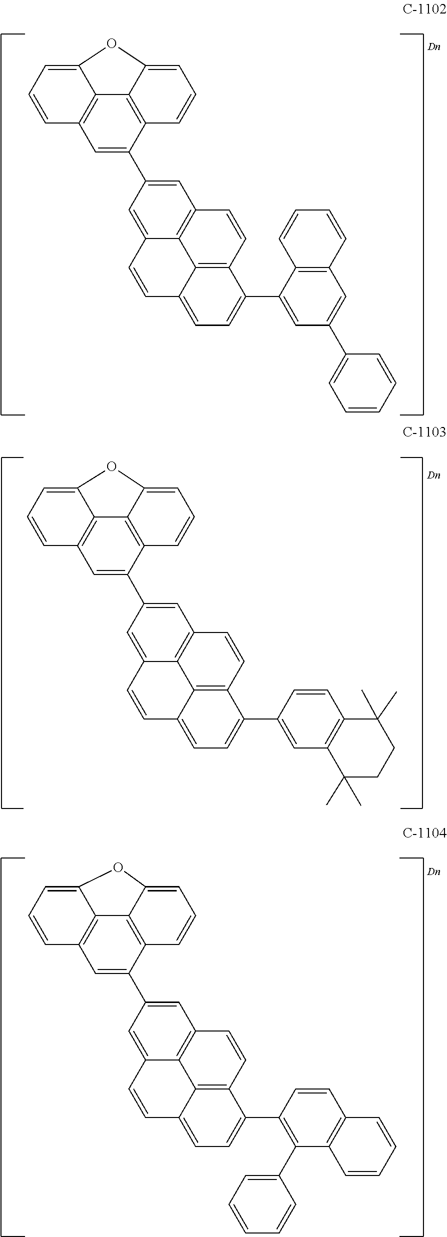 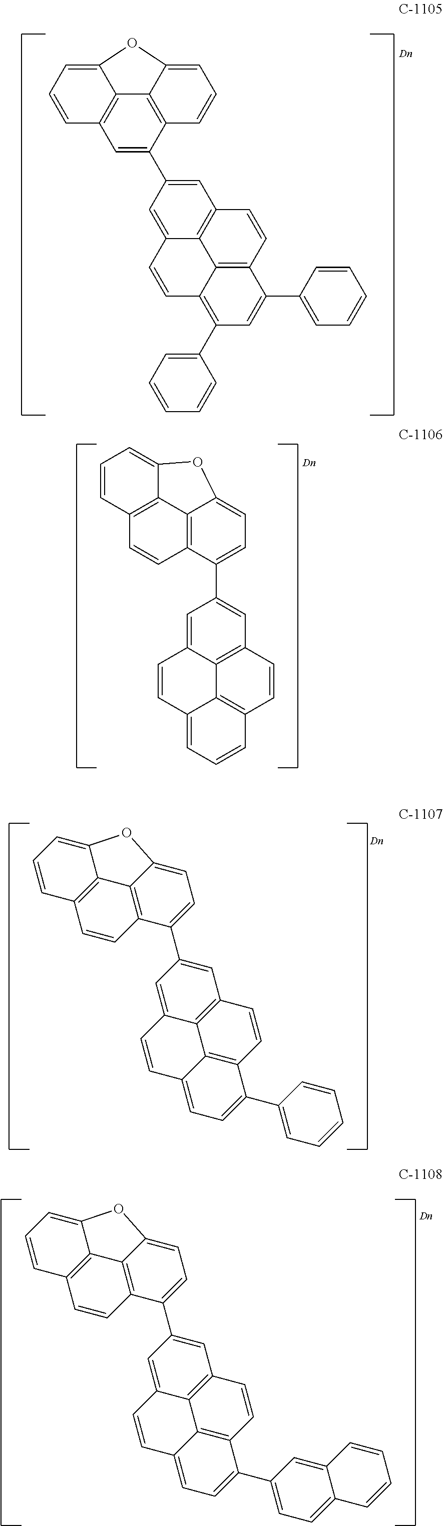 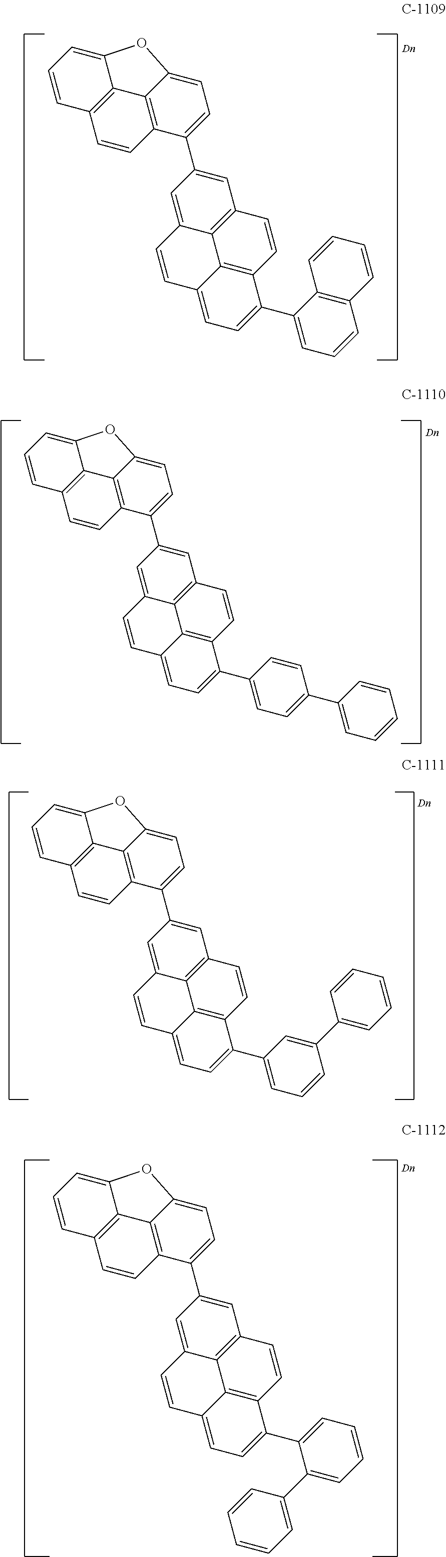 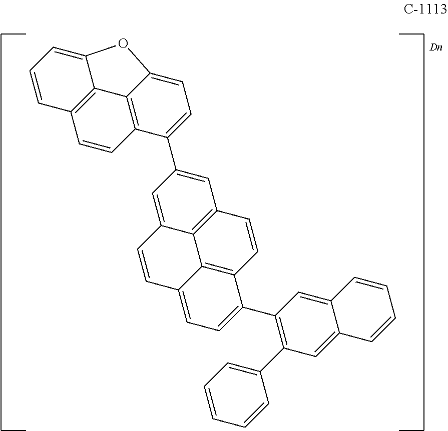 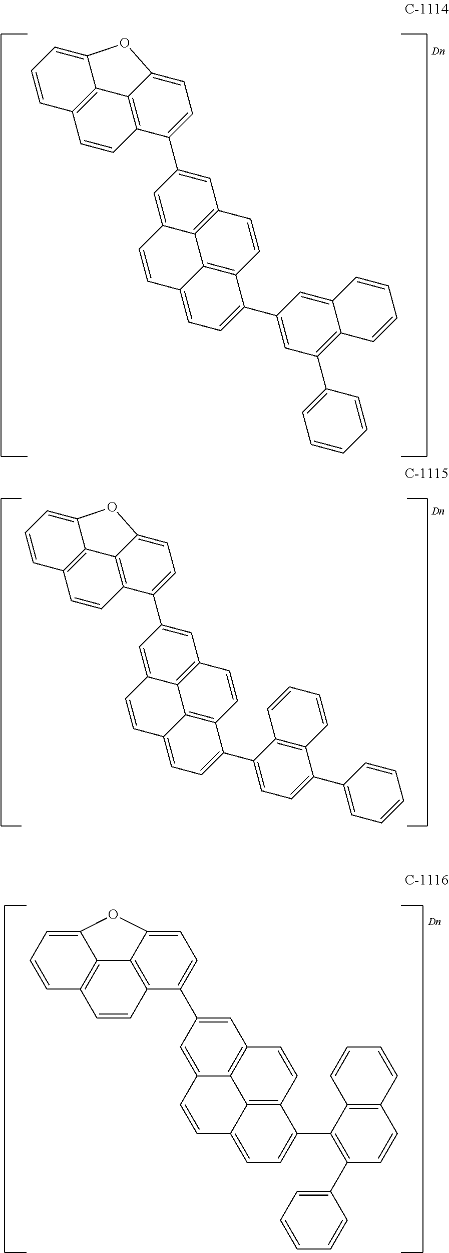 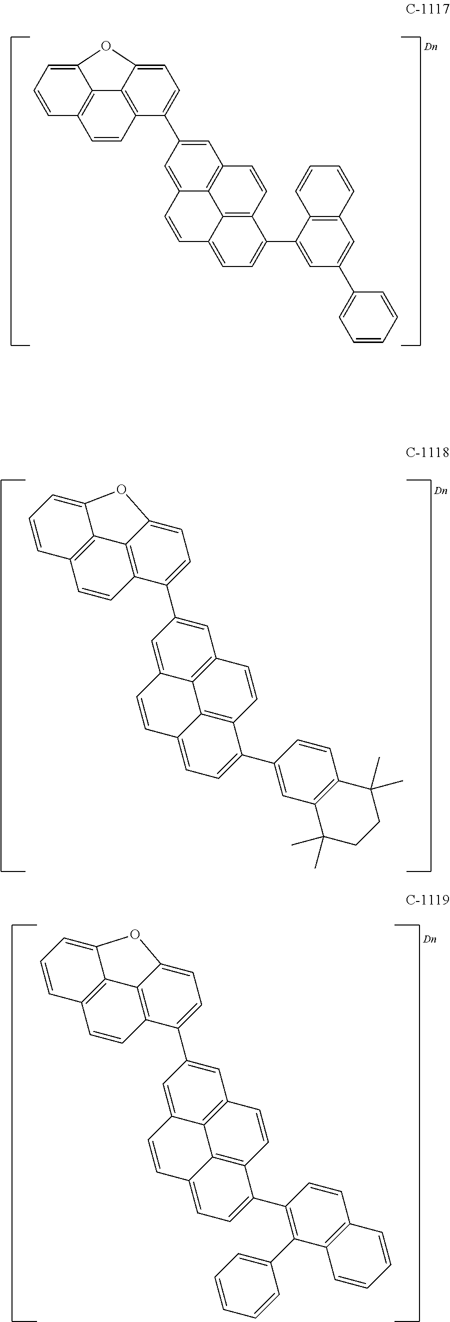 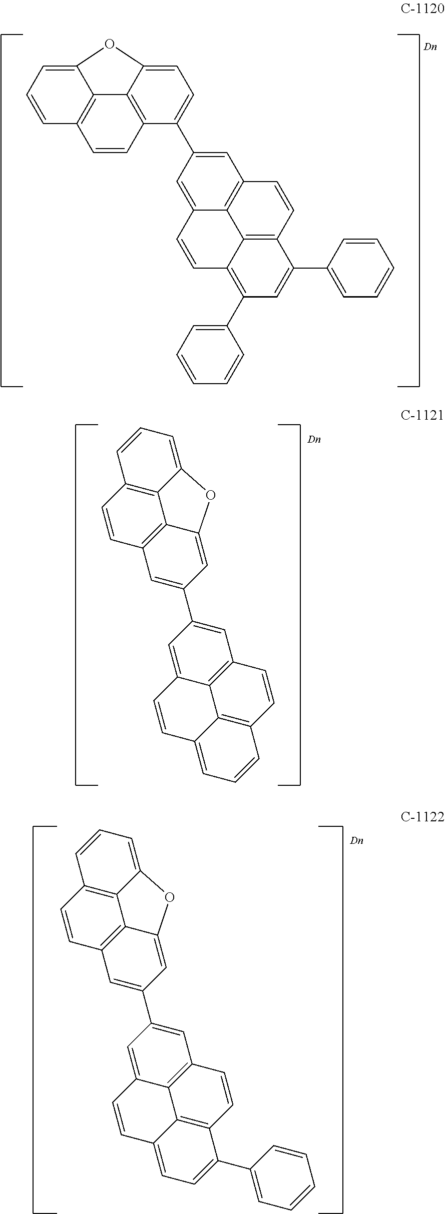 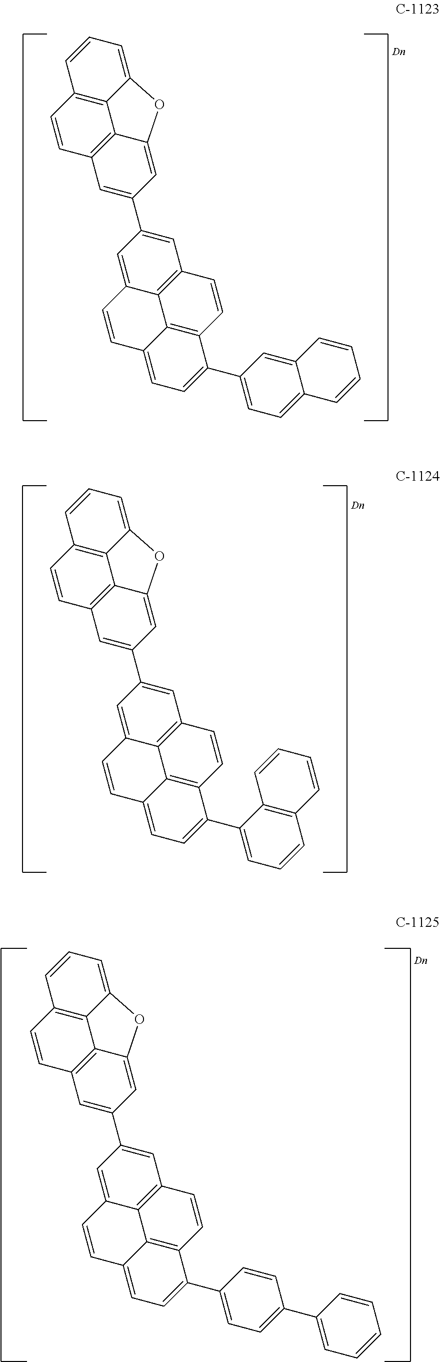 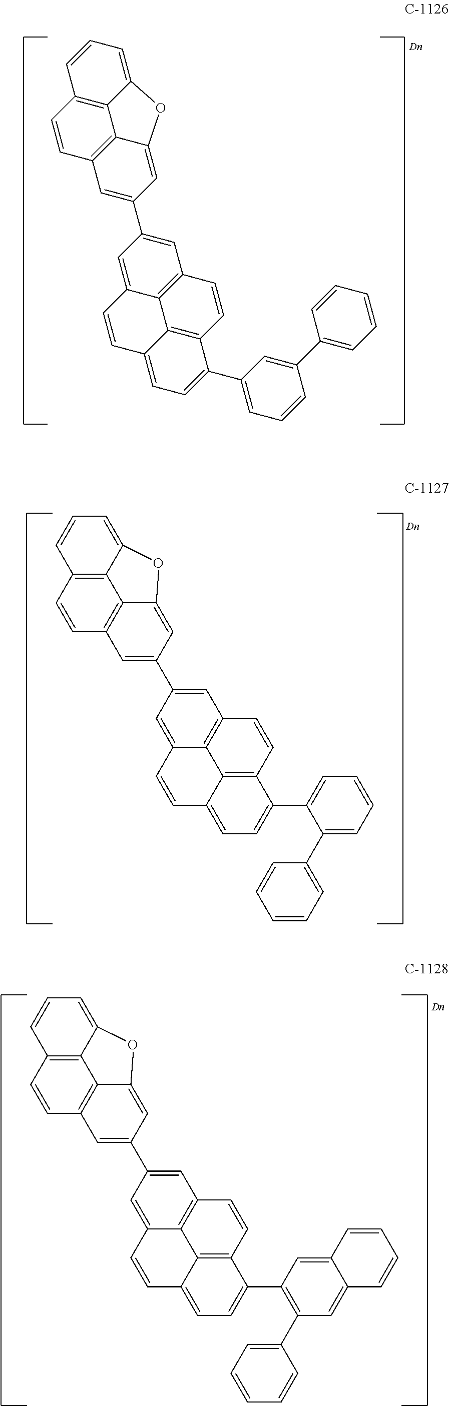 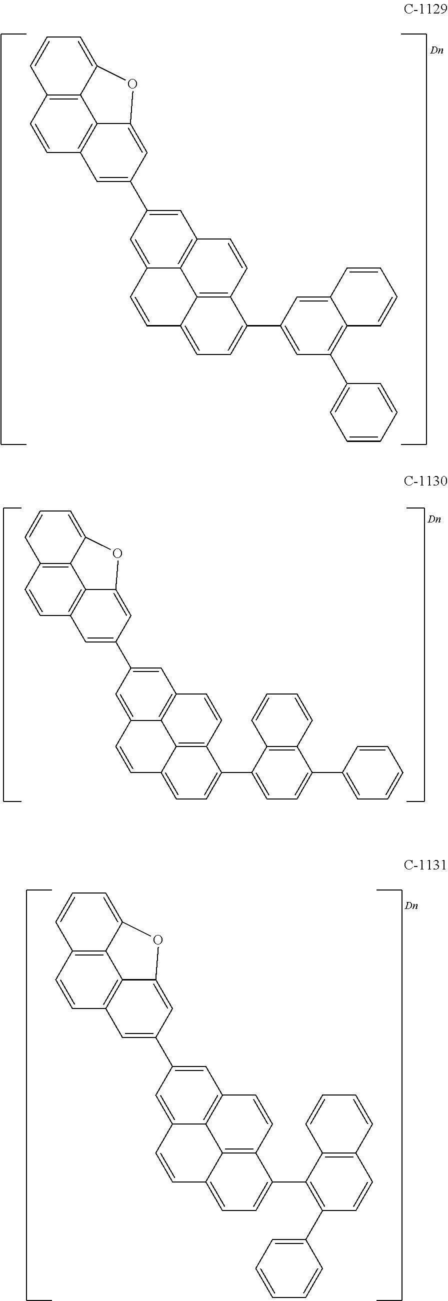 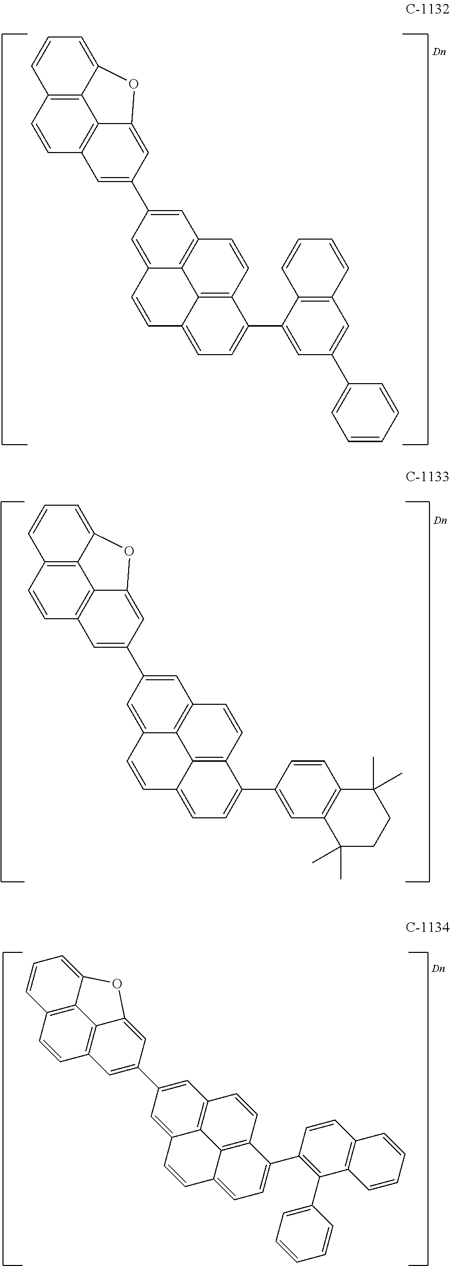 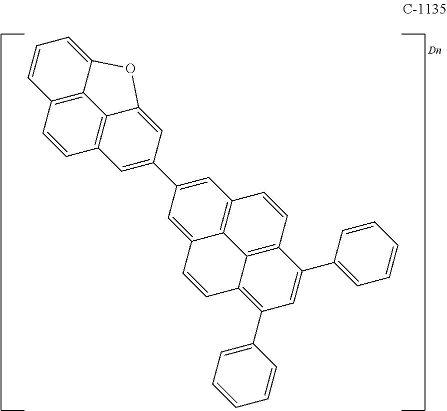 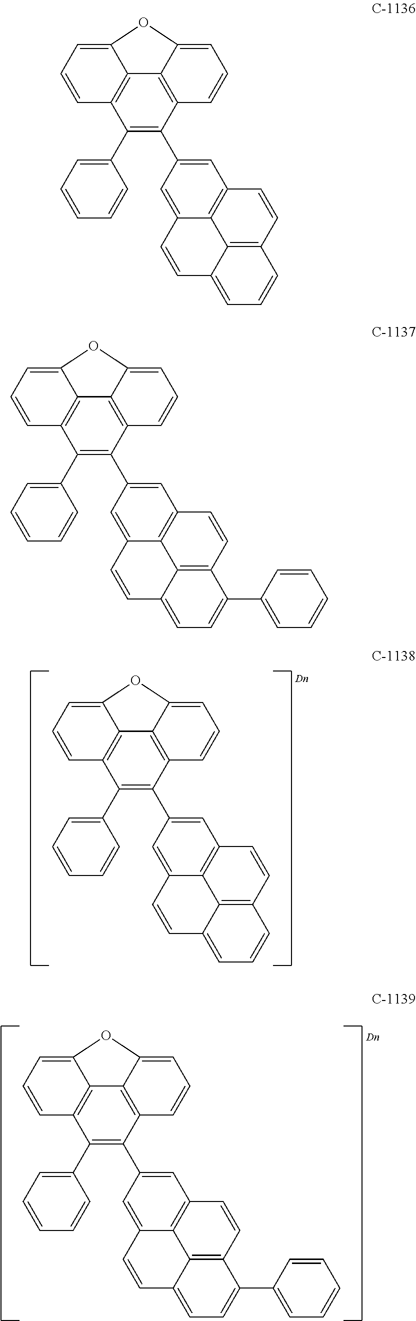 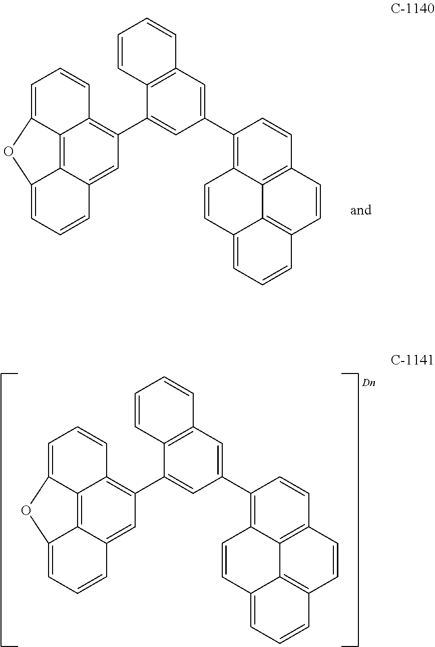
In the compounds, Dₙ indicates that n hydrogen atoms are replaced with deuterium atoms, wherein n is an integer ranging from 1 to the maximum number of hydrogen atoms present in the compound.

6. An organic electroluminescent device comprising:
a first electrode;
a second electrode opposing the first electrode; and
a plurality of light-emitting layers disposed between the first electrode and the second electrode,
wherein the light-emitting layers include a first light-emitting layer; and a second light-emitting layer, which are adjacent to each other, and
wherein the first light-emitting layer includes an organic electroluminescent compound according to claim 1 as a first host compound.

7. The organic electroluminescent device according to claim 6, wherein the second light-emitting layer comprises an organic electroluminescent compound represented by the following Formula 2 as a second host compound: 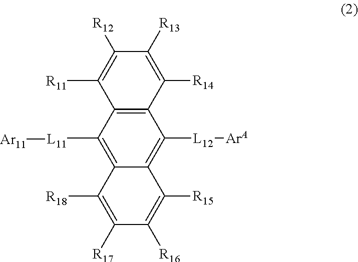
wherein,
R₁₁ to R₁₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of a (C₃-C₃₀) aliphatic ring and a (C₆-C₃₀) aromatic ring, or -L₁₃-N(Ar₁₃)(Ar₁₄);
L₁₁ and L₁₂ each independently represent a single bond, a substituted or unsubstituted (C₆-C₃₀)arylene, or a substituted or unsubstituted (3- to 30-membered)heteroarylene;
Ar₁₁ represents a substituted or unsubstituted (C₆-C₃₀)aryl or a substituted or unsubstituted (3- to 30-membered)heteroaryl; and
Ar^{A} represents a substituted or unsubstituted (C₆-C₃₀)aryl, a substituted or unsubstituted (3- to 30-membered)heteroaryl, or is represented by the following Formula A-1: 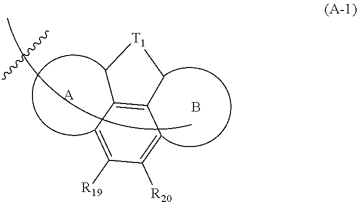
wherein,
T₁ represents —O—, —S—, —CRₐR_{b}, or —NR_{c};
ring A and ring B each independently represent a substituted or unsubstituted (C₆-C₃₀) arene ring, or a substituted or unsubstituted (3- to 30-membered)heteroarene ring;
R₁₉ and R₂₀ each independently represent a site linked to L₁₂, or hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of a (C₃-C₃₀) aliphatic ring and a (C₆-C₃₀) aromatic ring, or -L₁₃-N(Ar₁₃)(Ar₁₄);
Rₐ and R_{b} each independently represent a substituted or unsubstituted (C₁-C₃₀)alkyl, or a substituted or unsubstituted (C₆-C₃₀)aryl; or may be linked to each other to form a ring;
R_{c} represents a substituted or unsubstituted (C₁-C₃₀)alkyl or a substituted or unsubstituted (C₆-C₃₀)aryl;
L₁₃ represents a single bond, a substituted or unsubstituted (C₆-C₃₀)arylene, or a substituted or unsubstituted (3- to 30-membered)heteroarylene; and
Ar₁₃ and Ar₁₄ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted fused ring of a (C₃-C₃₀) aliphatic ring and a (C₆-C₃₀) aromatic ring, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl.

8. The organic electroluminescent device according to claim 7, wherein Ar₁₁ represents phenyl unsubstituted or substituted with deuterium, biphenyl unsubstituted or substituted with deuterium, terphenyl unsubstituted or substituted with deuterium, naphthyl unsubstituted or substituted with deuterium, phenanthrenyl unsubstituted or substituted with deuterium, or a combination thereof.

9. The organic electroluminescent device according to claim 7, wherein Ar^{A} represents phenyl unsubstituted or substituted with deuterium, biphenyl unsubstituted or substituted with deuterium, terphenyl unsubstituted or substituted with deuterium, naphthyl unsubstituted or substituted with deuterium, phenanthrenyl unsubstituted or substituted with deuterium, dibenzofuranyl unsubstituted or substituted with deuterium, dibenzothiophenyl unsubstituted or substituted with deuterium, or a combination thereof.

10. The organic electroluminescent device according to claim 7, wherein ring A and ring B in Formula A-1 each independently represent a benzene ring or a naphthalene ring.

11. The organic electroluminescent device according to claim 7, wherein at least one of the first host compound and the second host compound comprises deuterium.

12. The organic electroluminescent device according to claim 7, wherein the organic electroluminescent compound represented by Formula 2 is selected from the following compounds: 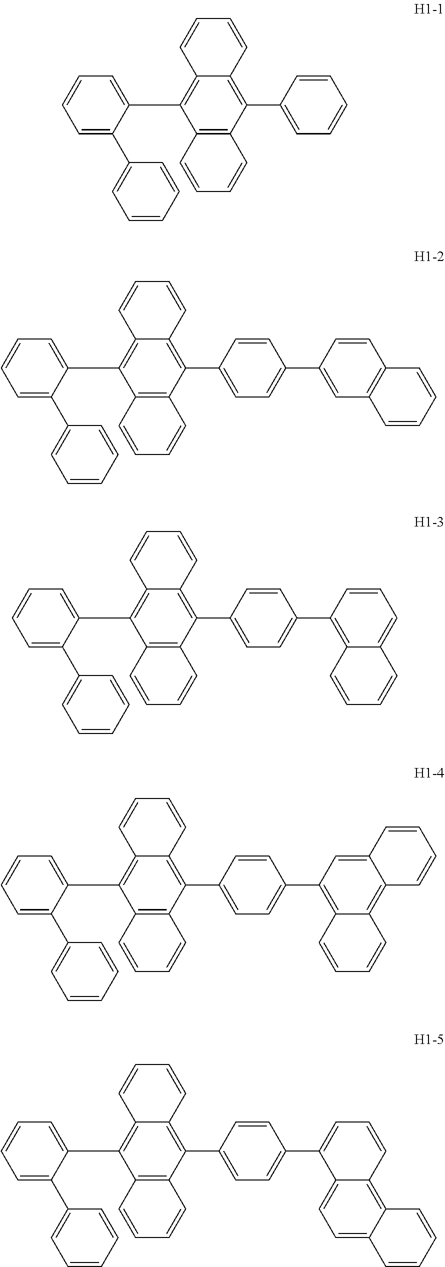 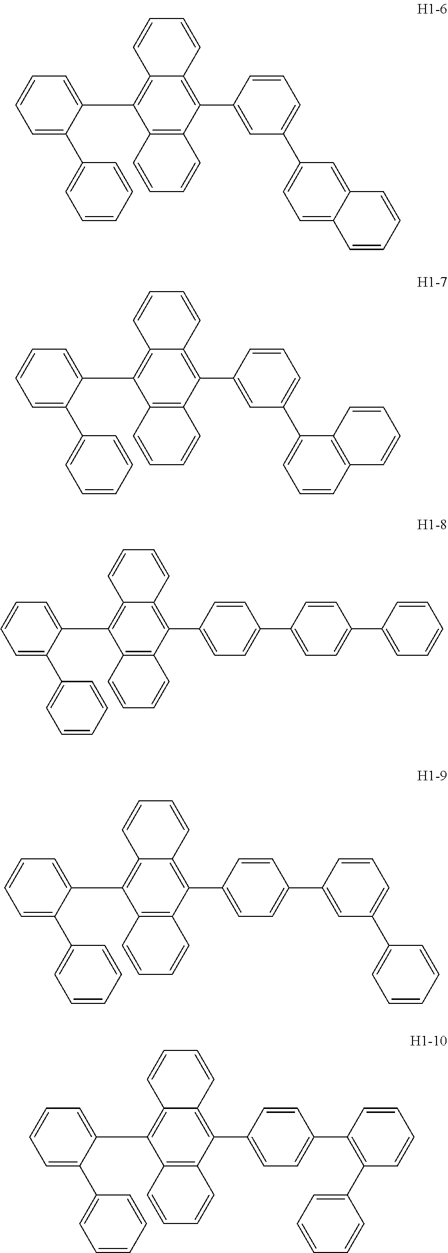 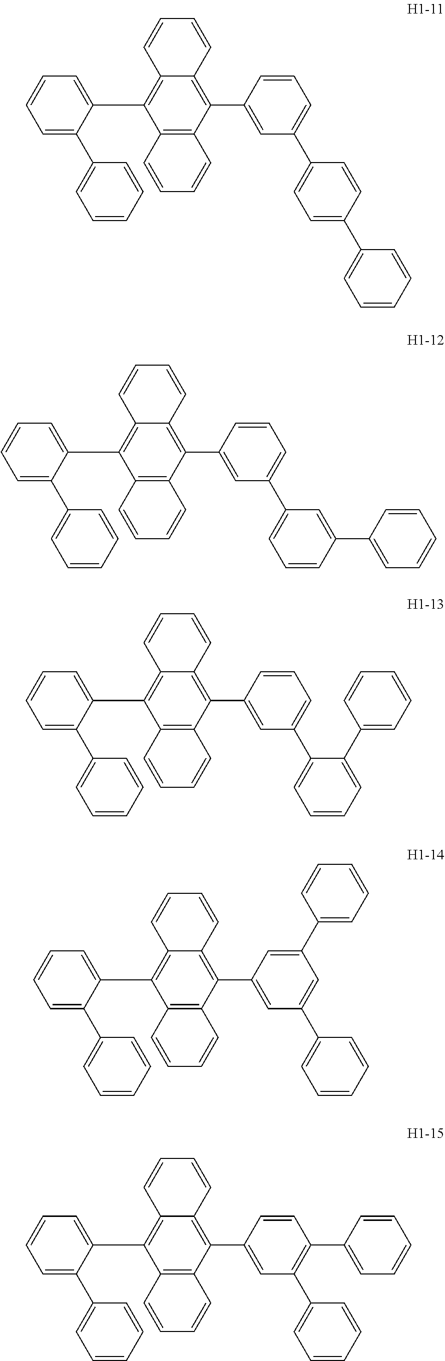 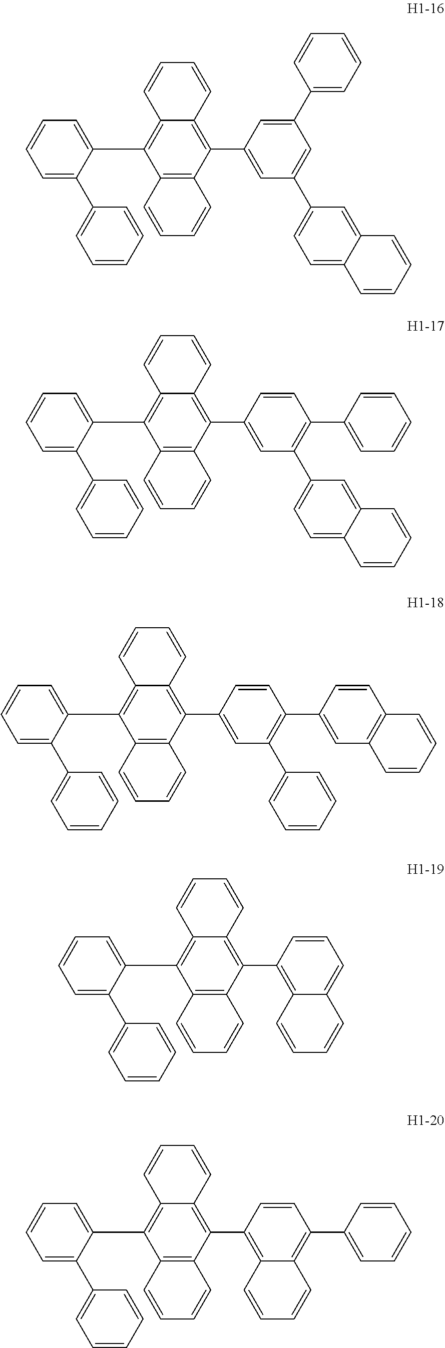 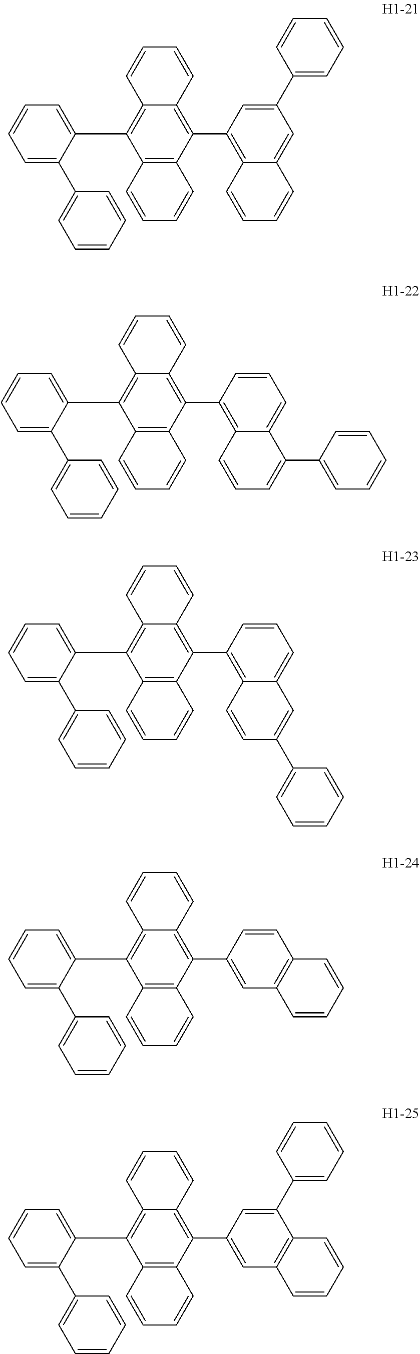 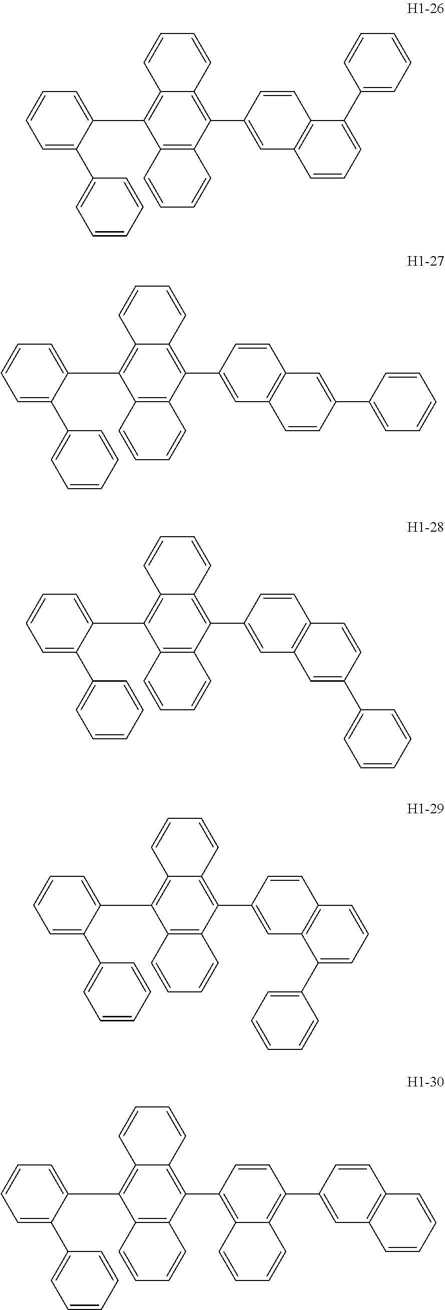 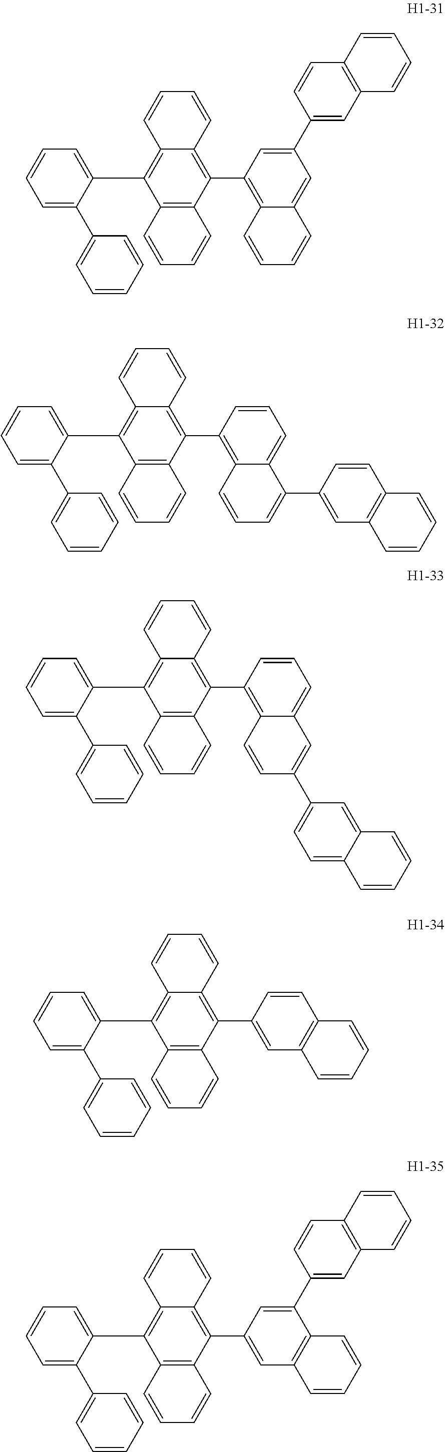 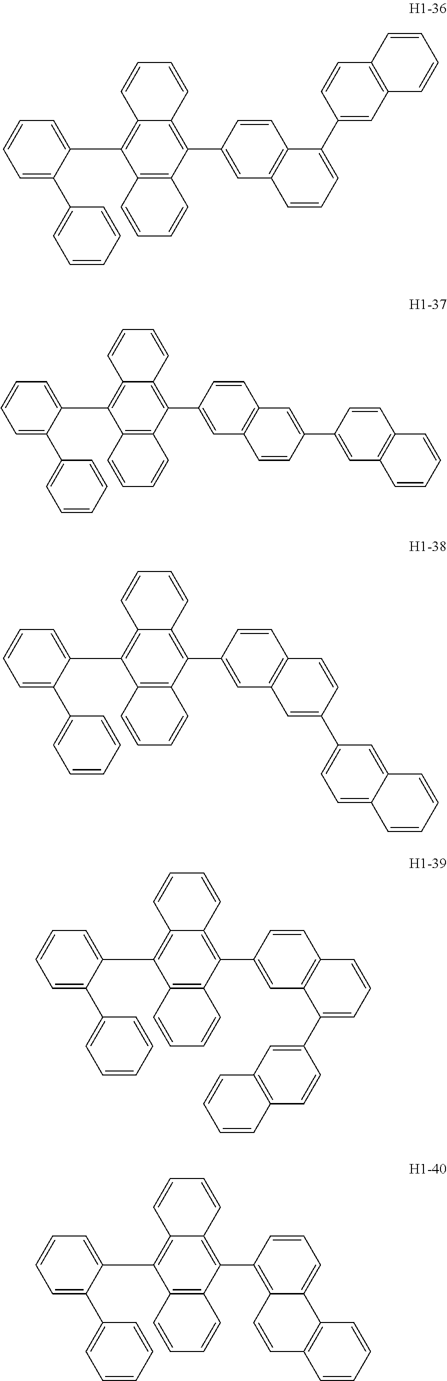 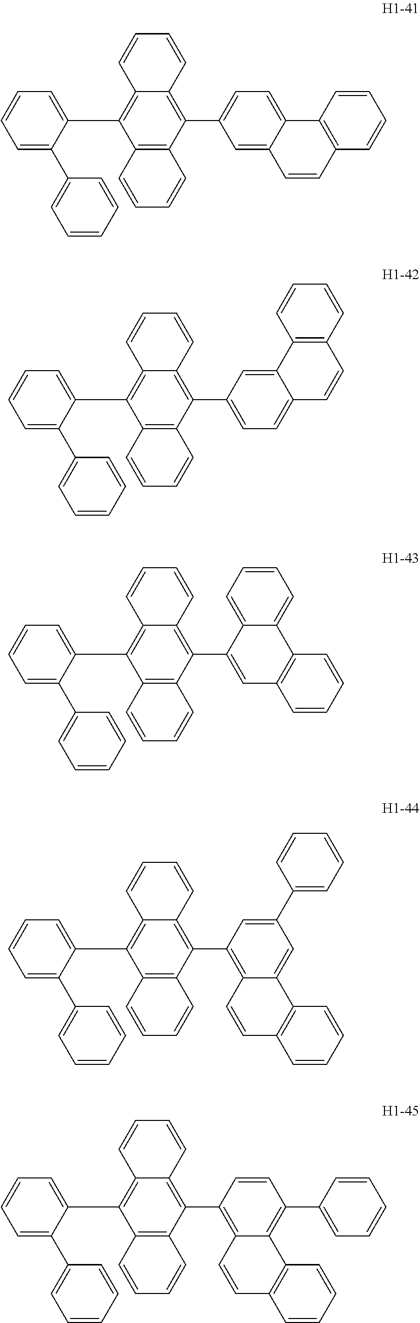 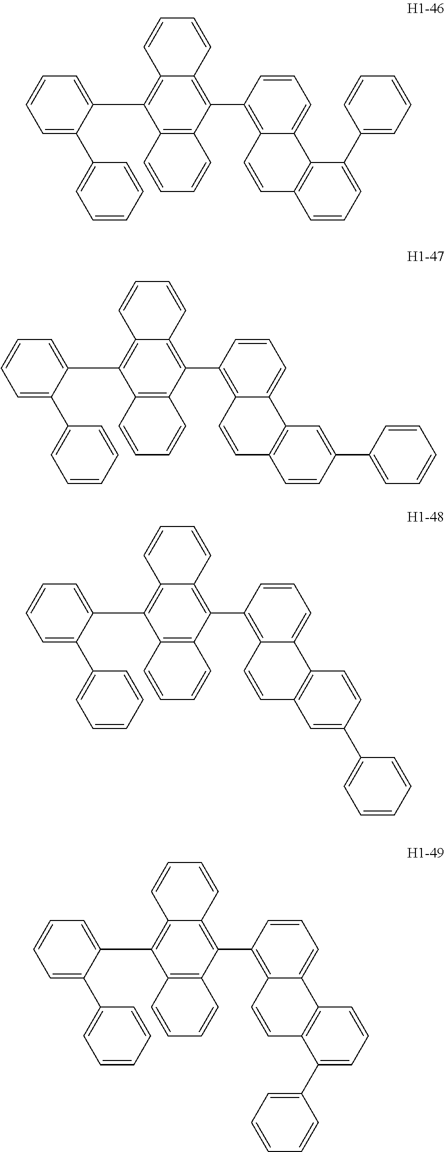 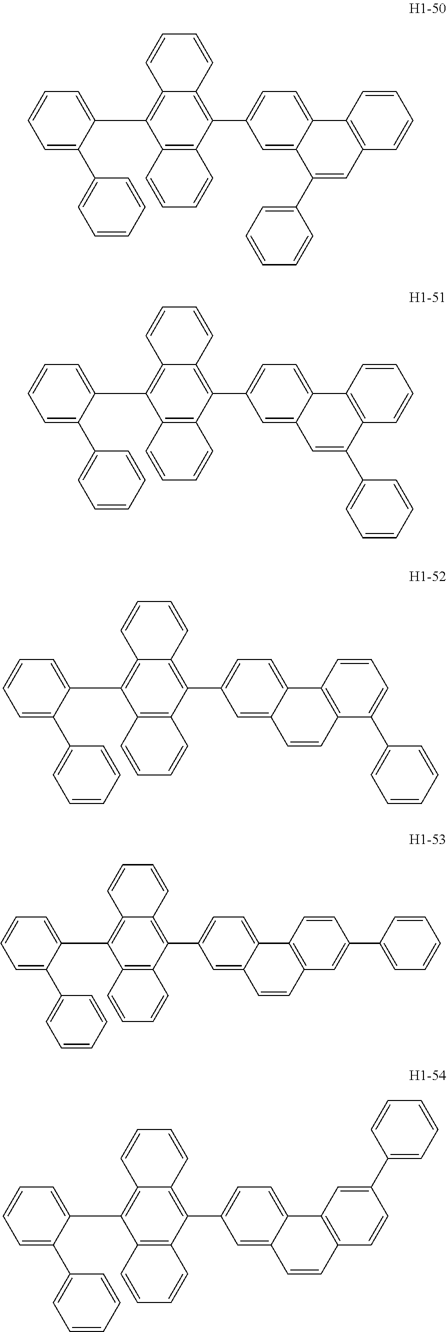 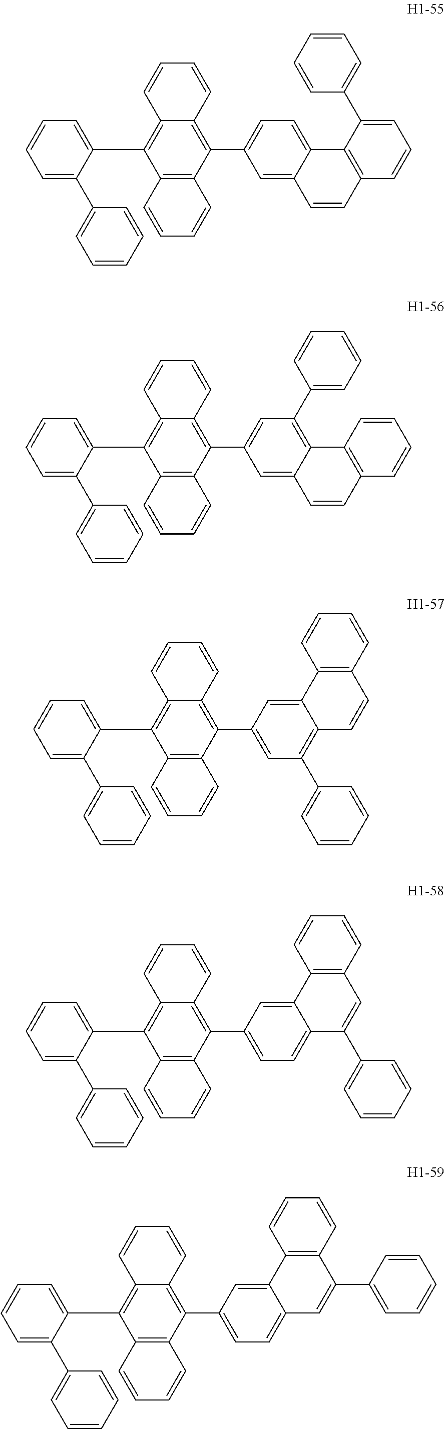 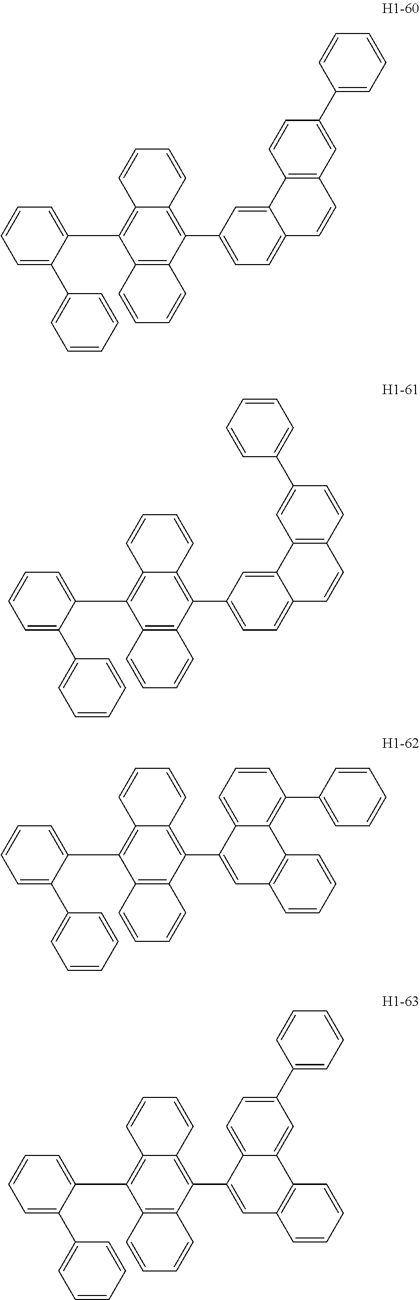 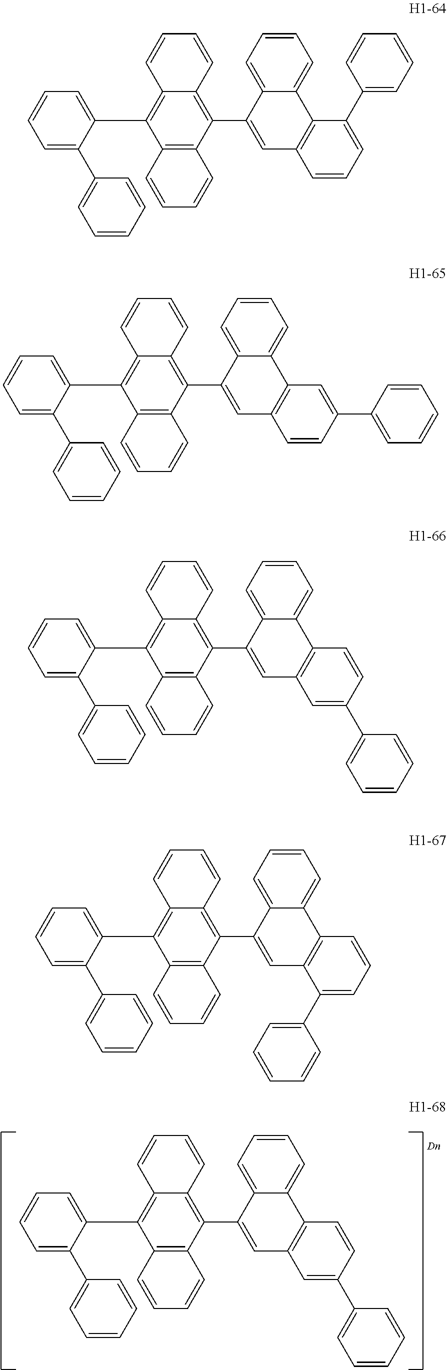 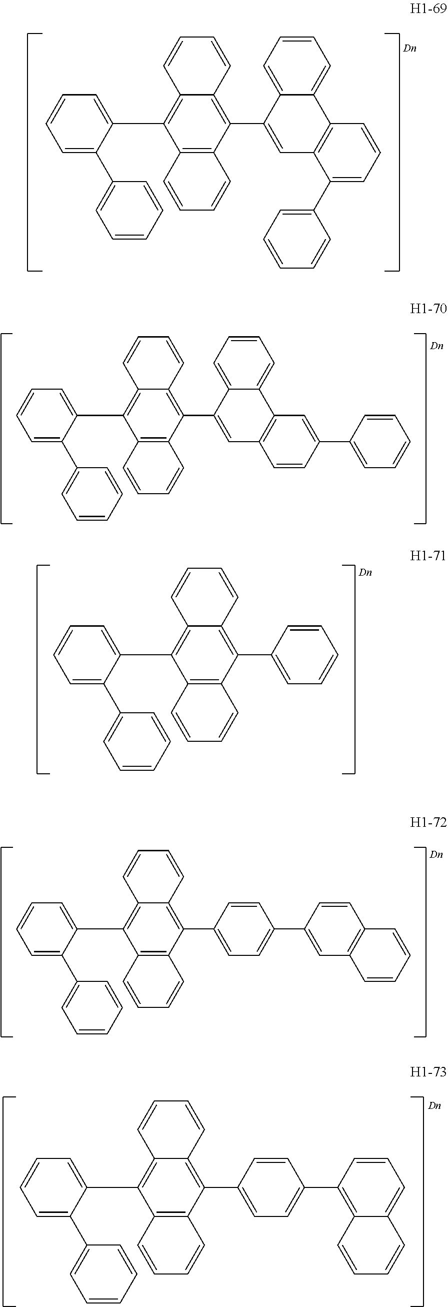 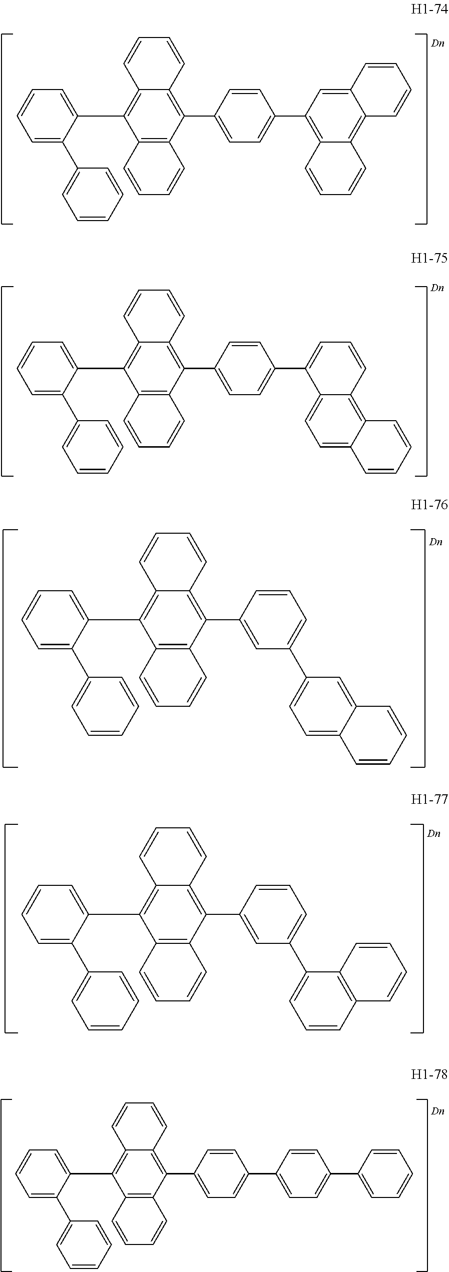 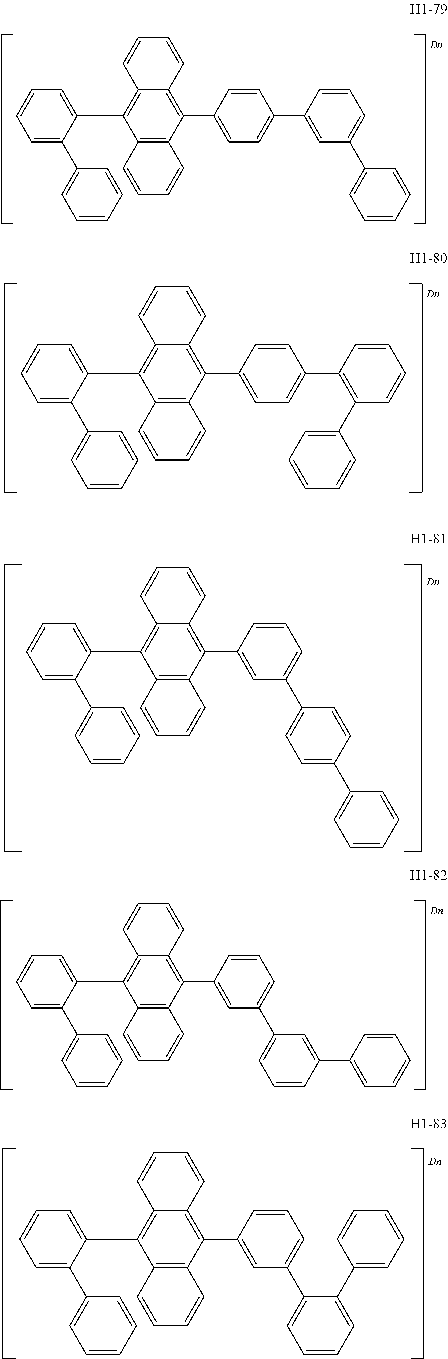 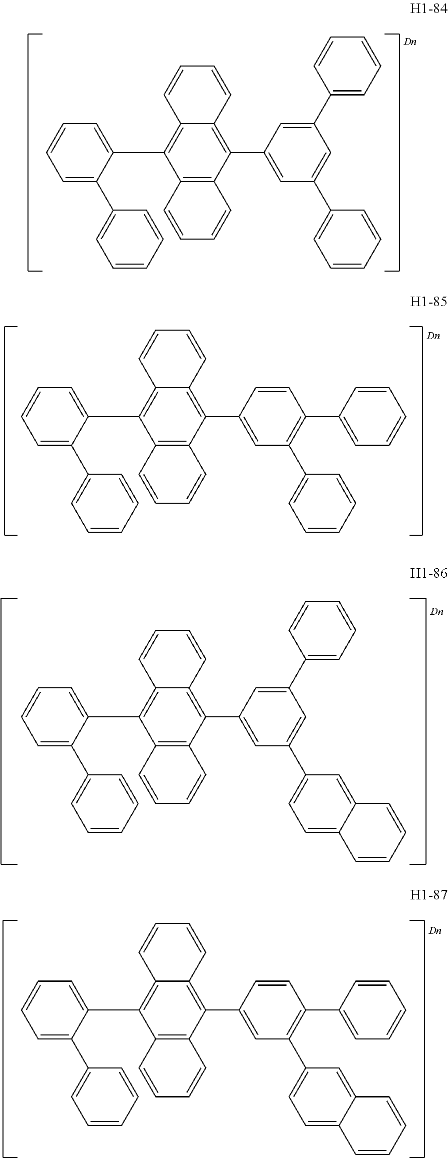 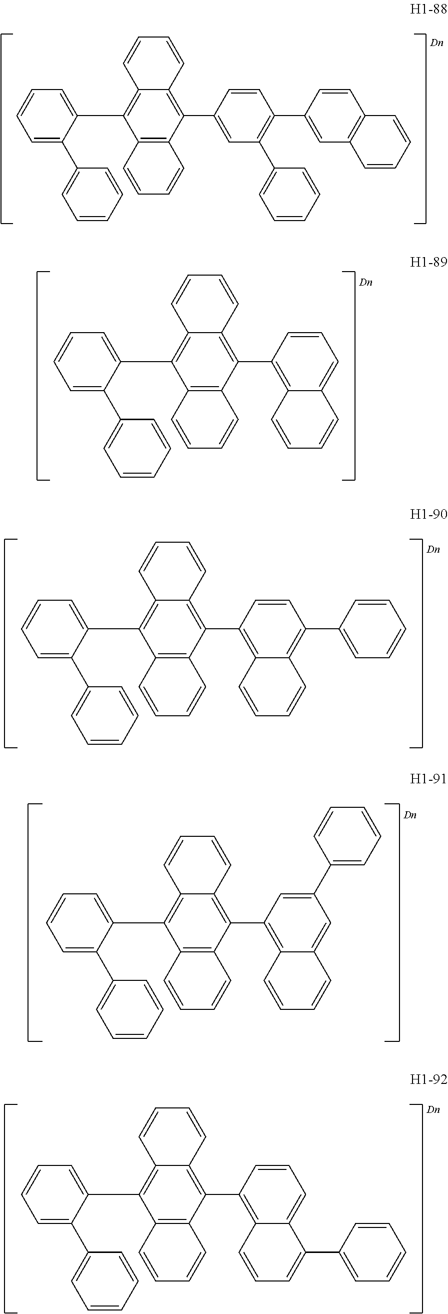 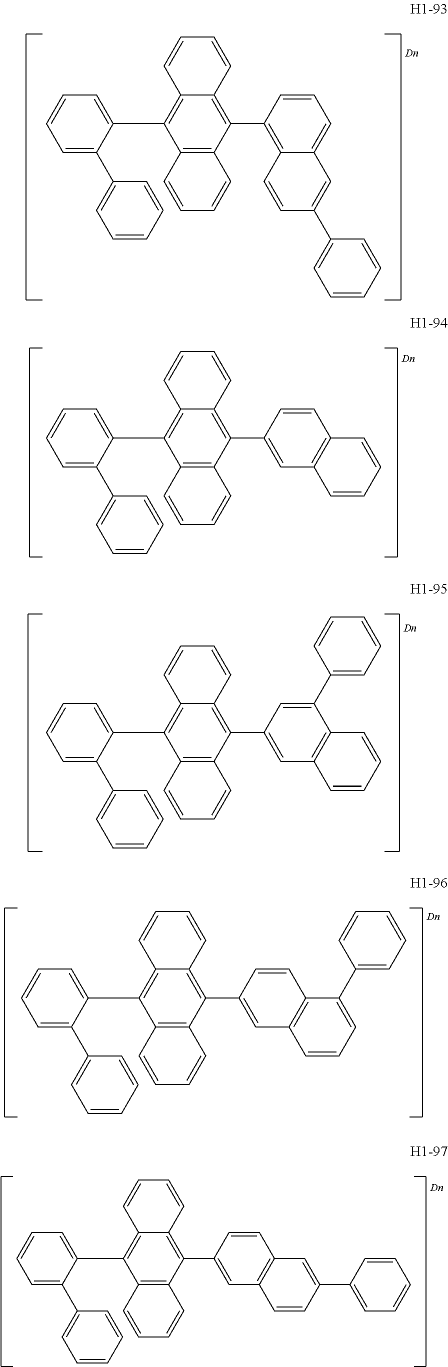 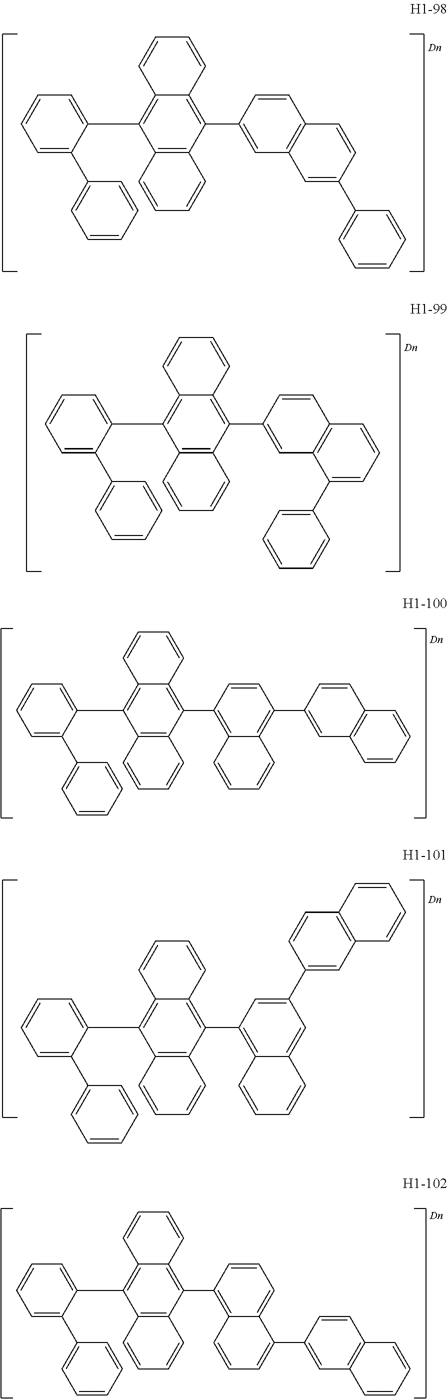 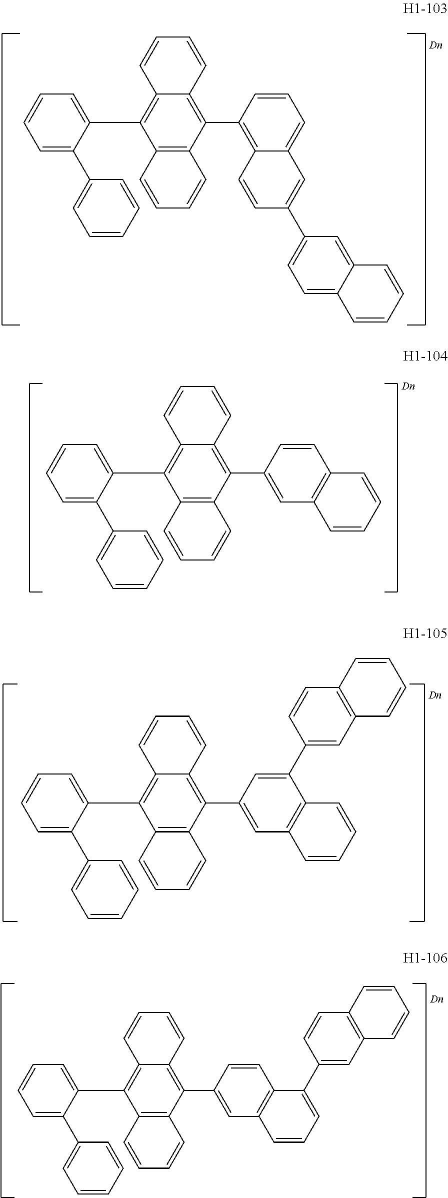 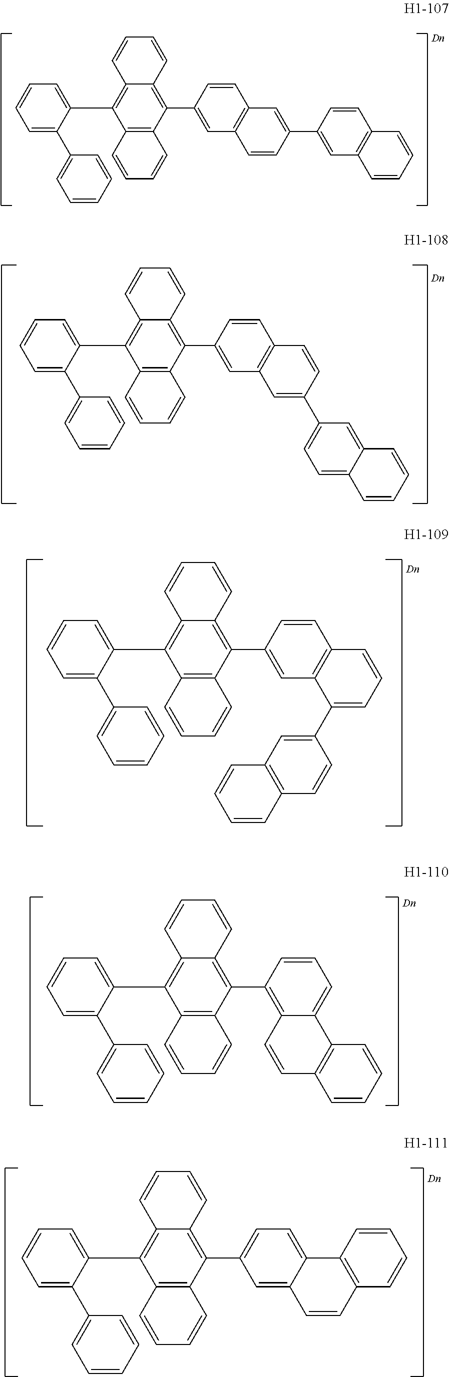 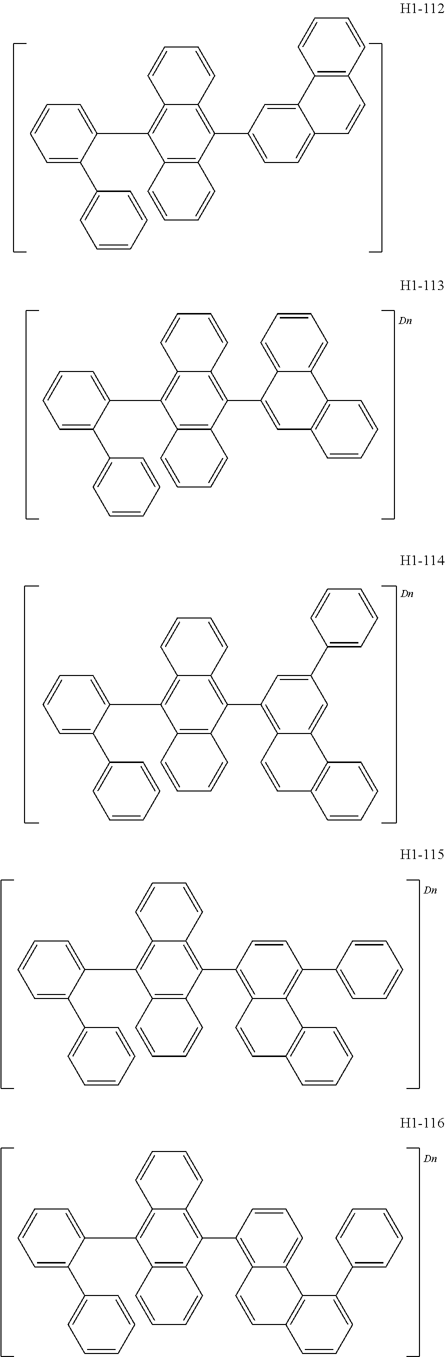 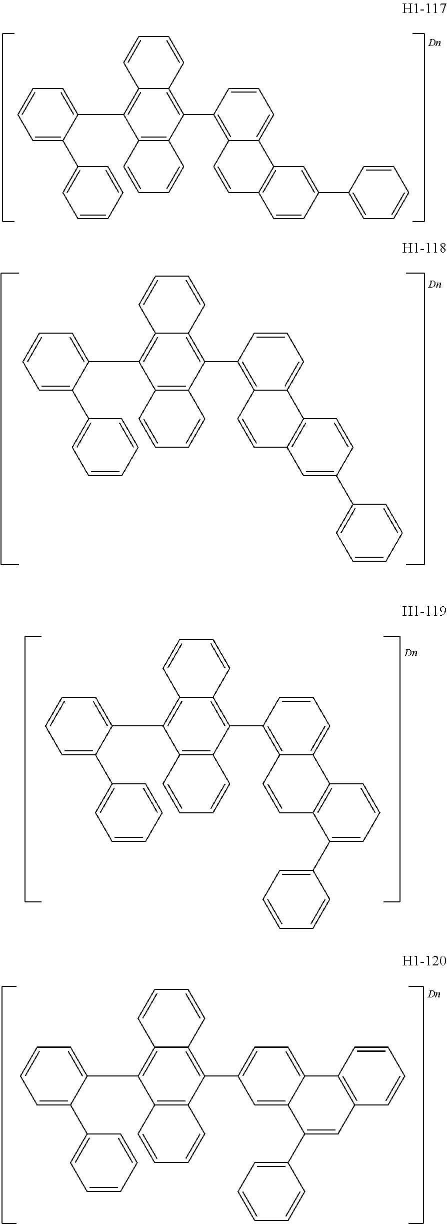 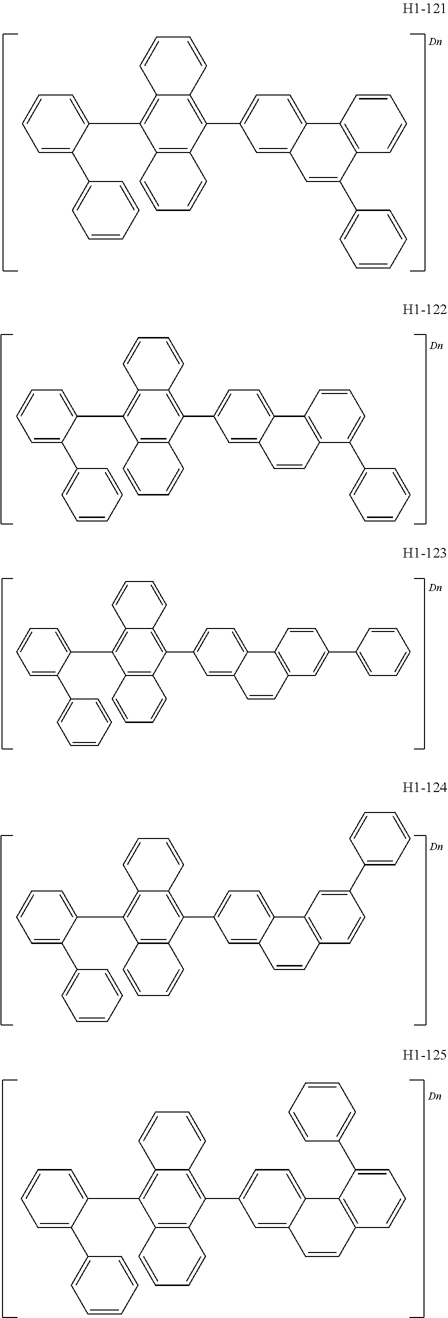 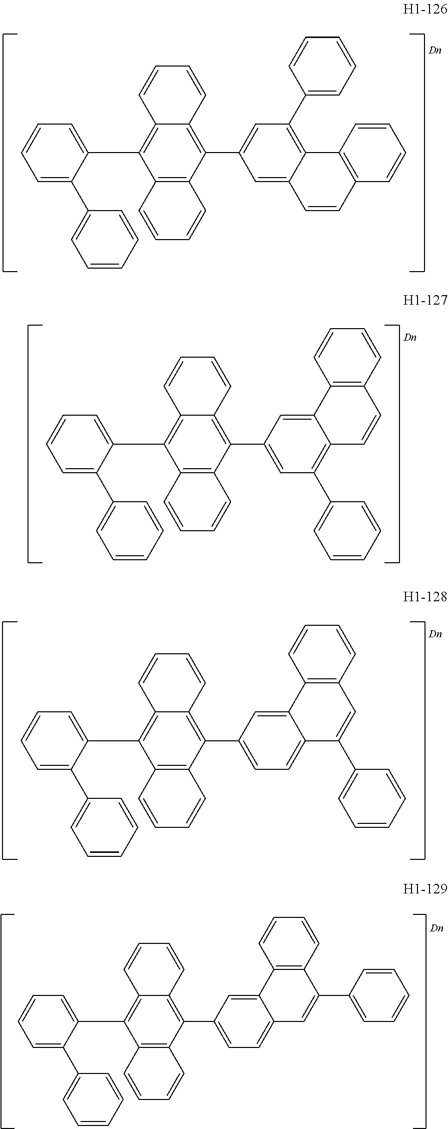 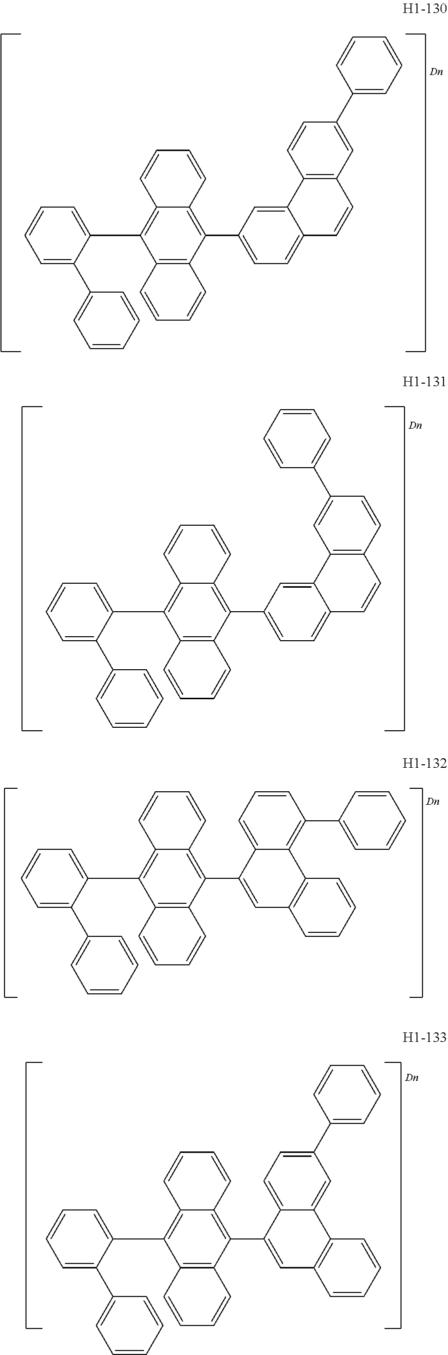 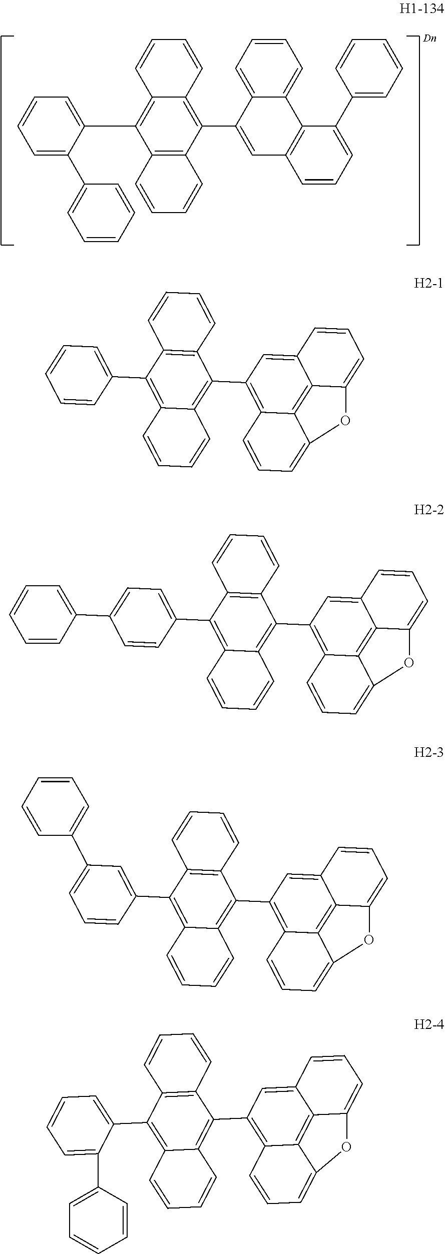 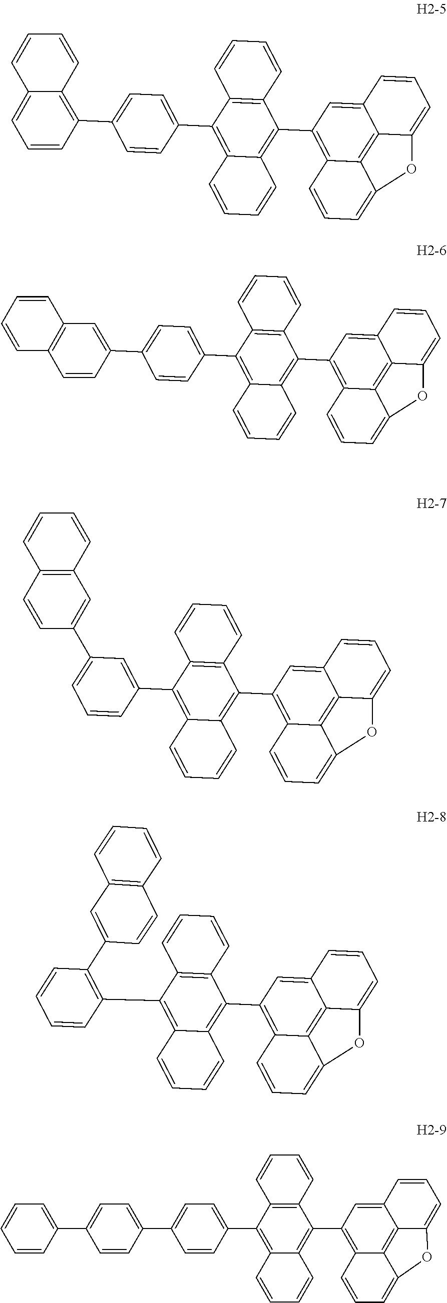 [SMILES: c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1 ; c1cc(-c2ccc3ccccc3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccccc3c2)c1 ; c1ccc(-c2ccc(-c3ccc(-c4c5ccccc5c(-c5cc6cccc7oc8cccc5c8c67)c5ccccc45)cc3)cc2)cc1] 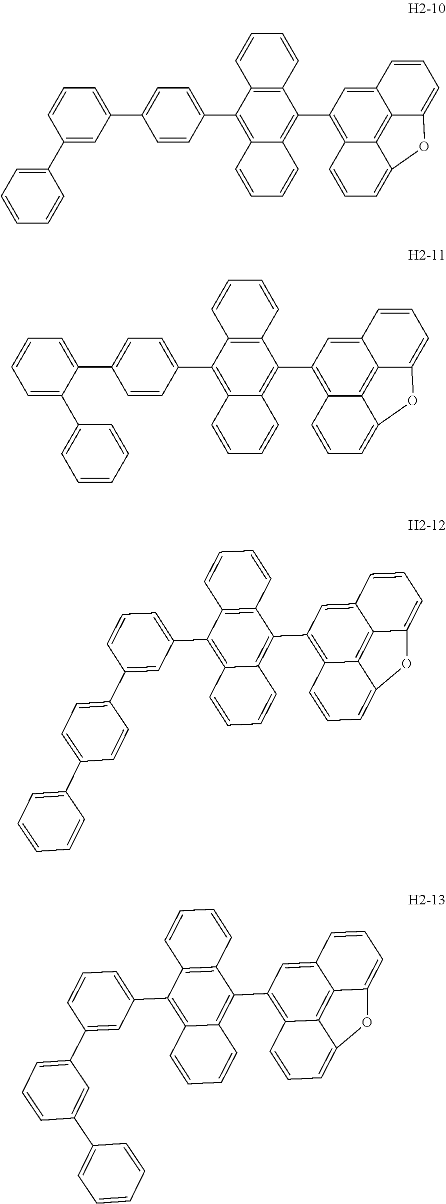 [SMILES: c1ccc(-c2cccc(-c3ccc(-c4c5ccccc5c(-c5cc6cccc7oc8cccc5c8c67)c5ccccc45)cc3)c2)cc1 ; c1ccc(-c2ccccc2-c2ccc(-c3c4ccccc4c(-c4cc5cccc6oc7cccc4c7c56)c4ccccc34)cc2)cc1 ; c1ccc(-c2ccc(-c3cccc(-c4c5ccccc5c(-c5cc6cccc7oc8cccc5c8c67)c5ccccc45)c3)cc2)cc1 ; c1ccc(-c2cccc(-c3cccc(-c4c5ccccc5c(-c5cc6cccc7oc8cccc5c8c67)c5ccccc45)c3)c2)cc1] 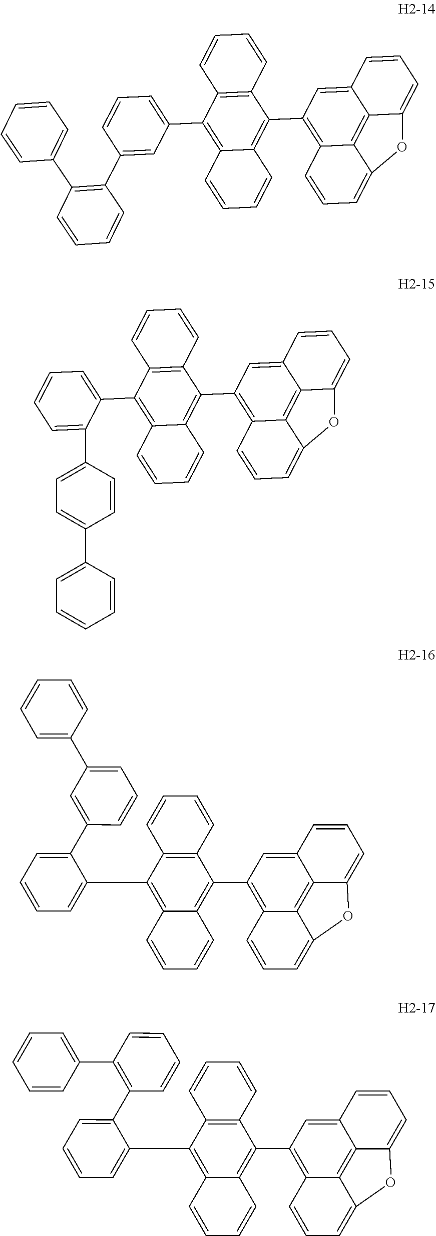 [SMILES: c1ccc(-c2ccccc2-c2cccc(-c3c4ccccc4c(-c4cc5cccc6oc7cccc4c7c56)c4ccccc34)c2)cc1 ; c1ccc(-c2ccc(-c3ccccc3-c3c4ccccc4c(-c4cc5cccc6oc7cccc4c7c56)c4ccccc34)cc2)cc1 ; c1ccc(-c2cccc(-c3ccccc3-c3c4ccccc4c(-c4cc5cccc6oc7cccc4c7c56)c4ccccc34)c2)cc1 ; c1ccc(-c2ccccc2-c2ccccc2-c2c3ccccc3c(-c3cc4cccc5oc6cccc3c6c45)c3ccccc23)cc1] 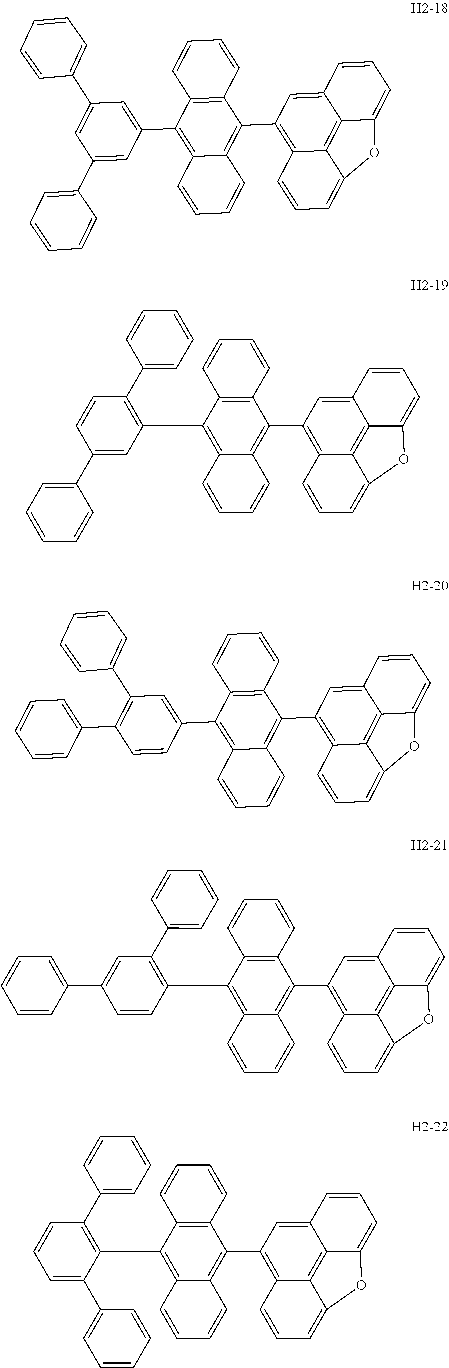 [SMILES: c1ccc(-c2cc(-c3ccccc3)cc(-c3c4ccccc4c(-c4cc5cccc6oc7cccc4c7c56)c4ccccc34)c2)cc1 ; c1ccc(-c2ccc(-c3ccccc3)c(-c3c4ccccc4c(-c4cc5cccc6oc7cccc4c7c56)c4ccccc34)c2)cc1 ; c1ccc(-c2ccc(-c3c4ccccc4c(-c4cc5cccc6oc7cccc4c7c56)c4ccccc34)cc2-c2ccccc2)cc1 ; c1ccc(-c2ccc(-c3c4ccccc4c(-c4cc5cccc6oc7cccc4c7c56)c4ccccc34)c(-c3ccccc3)c2)cc1 ; c1ccc(-c2cccc(-c3ccccc3)c2-c2c3ccccc3c(-c3cc4cccc5oc6cccc3c6c45)c3ccccc23)cc1] 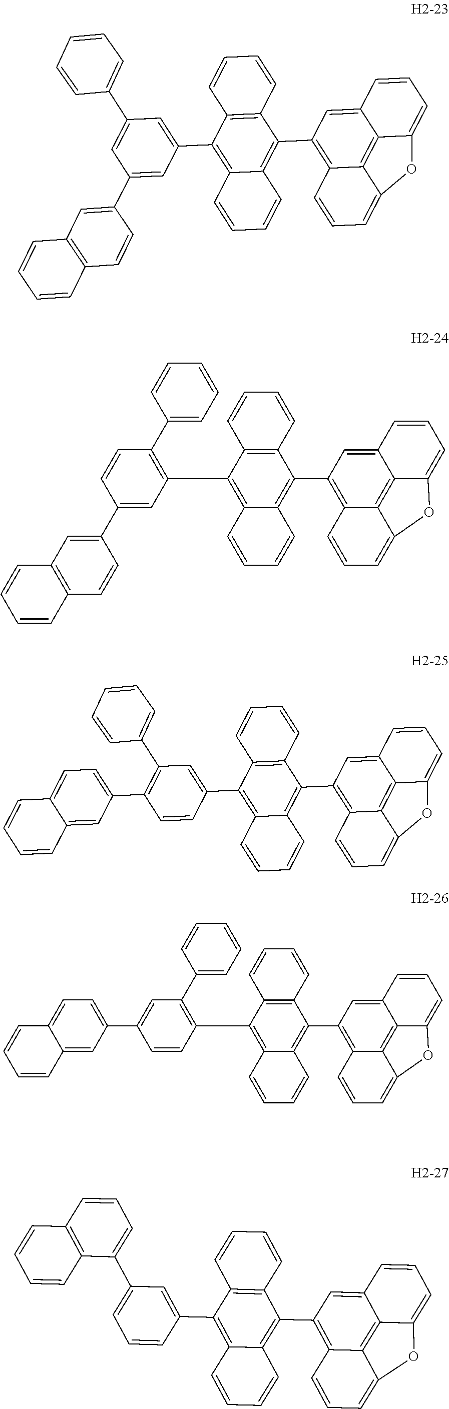 [SMILES: c1ccc(-c2cc(-c3ccc4ccccc4c3)cc(-c3c4ccccc4c(-c4cc5cccc6oc7cccc4c7c56)c4ccccc34)c2)cc1 ; c1ccc(-c2ccc(-c3ccc4ccccc4c3)cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)ccc2-c2ccc3ccccc3c2)cc1 ; c1ccc(-c2cc(-c3ccc4ccccc4c3)ccc2-c2c3ccccc3c(-c3cc4cccc5oc6cccc3c6c45)c3ccccc23)cc1 ; c1cc(-c2cccc3ccccc23)cc(-c2c3ccccc3c(-c3cc4cccc5oc6cccc3c6c45)c3ccccc23)c1] 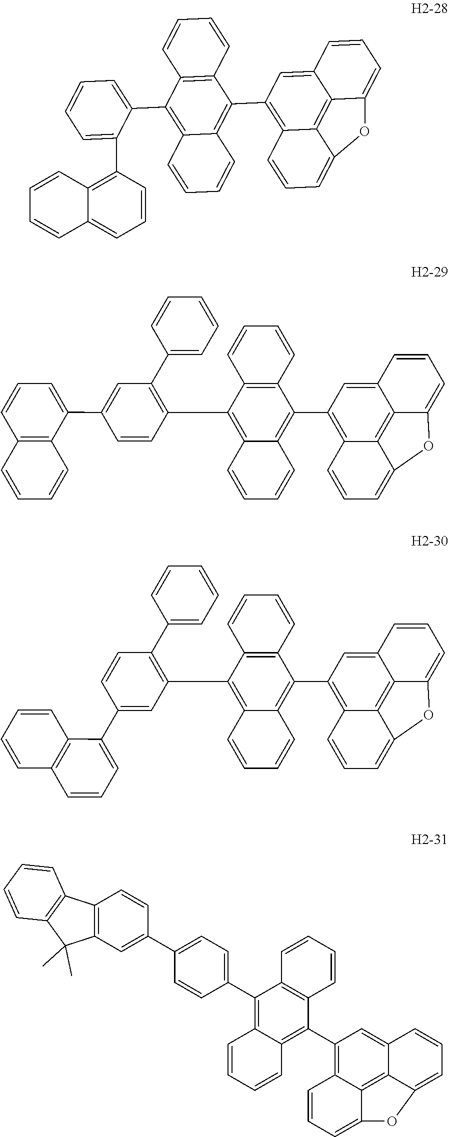 [SMILES: c1ccc(-c2c3ccccc3c(-c3cc4cccc5oc6cccc3c6c45)c3ccccc23)c(-c2cccc3ccccc23)c1 ; c1ccc(-c2cc(-c3cccc4ccccc34)ccc2-c2c3ccccc3c(-c3cc4cccc5oc6cccc3c6c45)c3ccccc23)cc1 ; c1ccc(-c2ccc(-c3cccc4ccccc34)cc2-c2c3ccccc3c(-c3cc4cccc5oc6cccc3c6c45)c3ccccc23)cc1 ; CC1(C)c2ccccc2-c2ccc(-c3ccc(-c4c5ccccc5c(-c5cc6cccc7oc8cccc5c8c67)c5ccccc45)cc3)cc21] 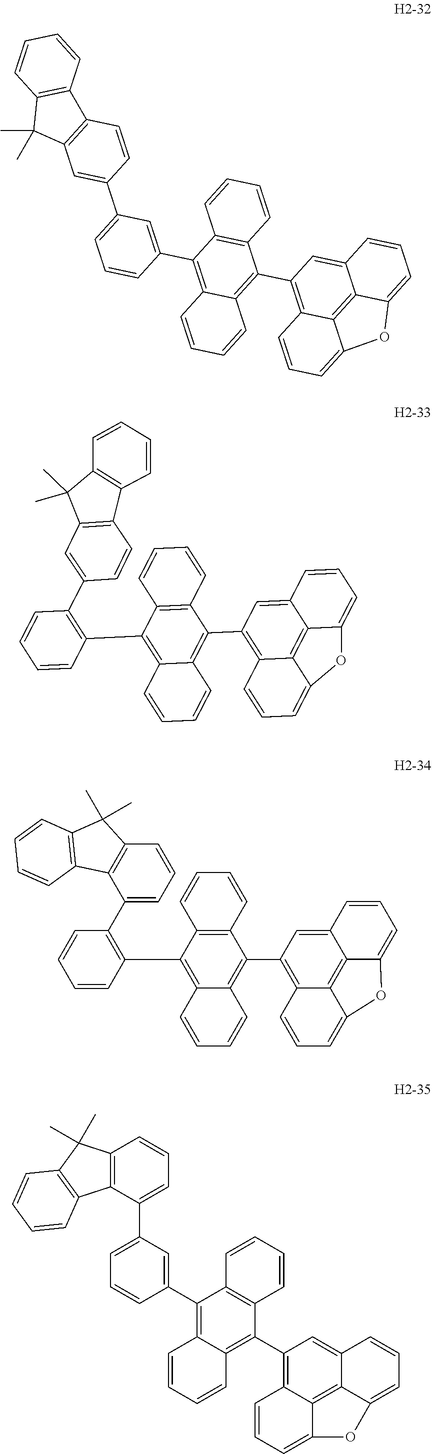 [SMILES: CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21] 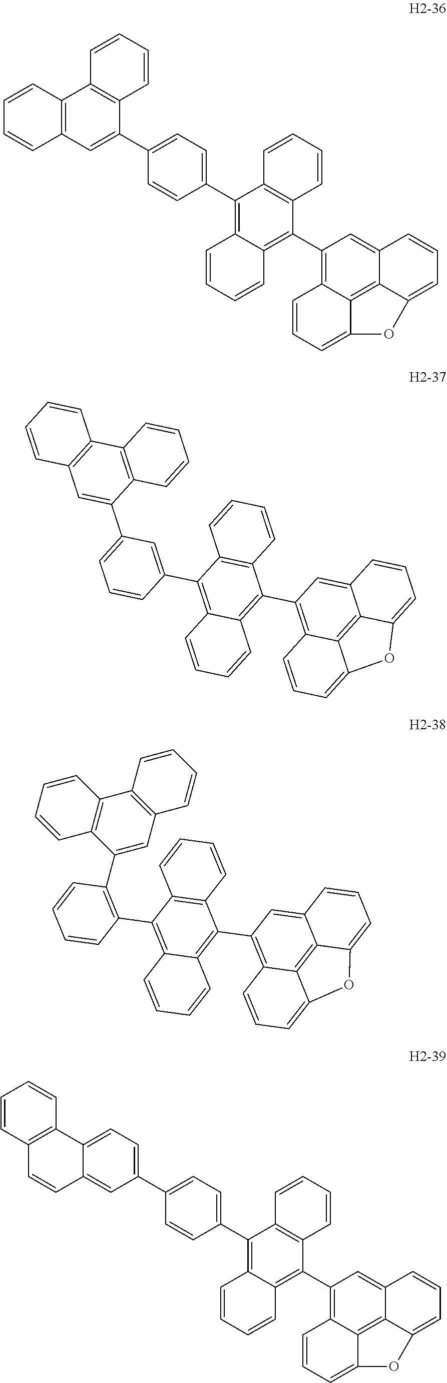 [SMILES: c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1 ; c1ccc2c(c1)ccc1cc(-c3ccc(-c4c5ccccc5c(-c5cc6cccc7oc8cccc5c8c67)c5ccccc45)cc3)ccc12] 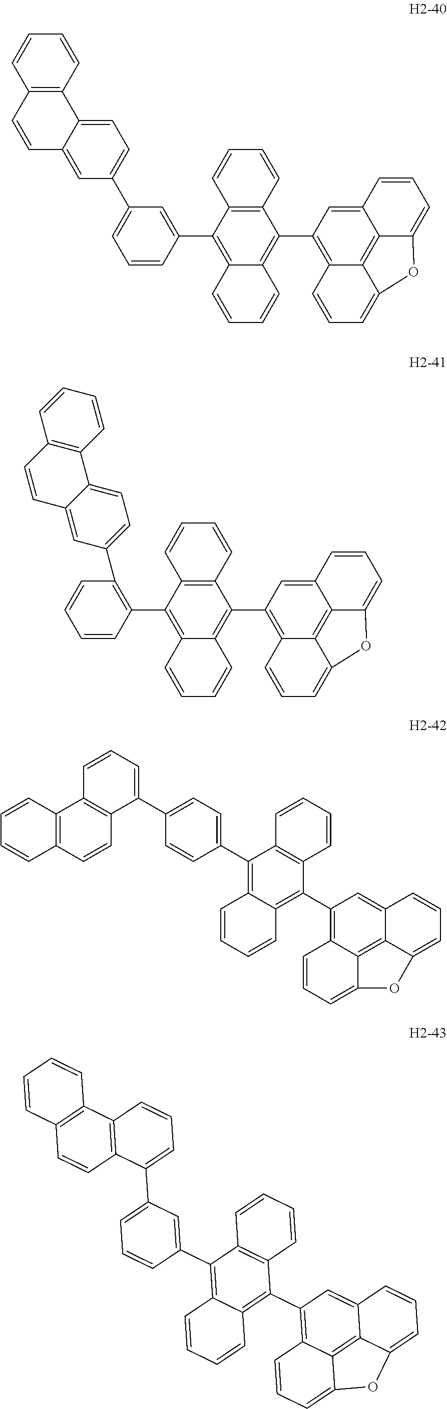 [SMILES: c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1 ; c1ccc2c(c1)ccc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2ccc2ccccc23)c1] 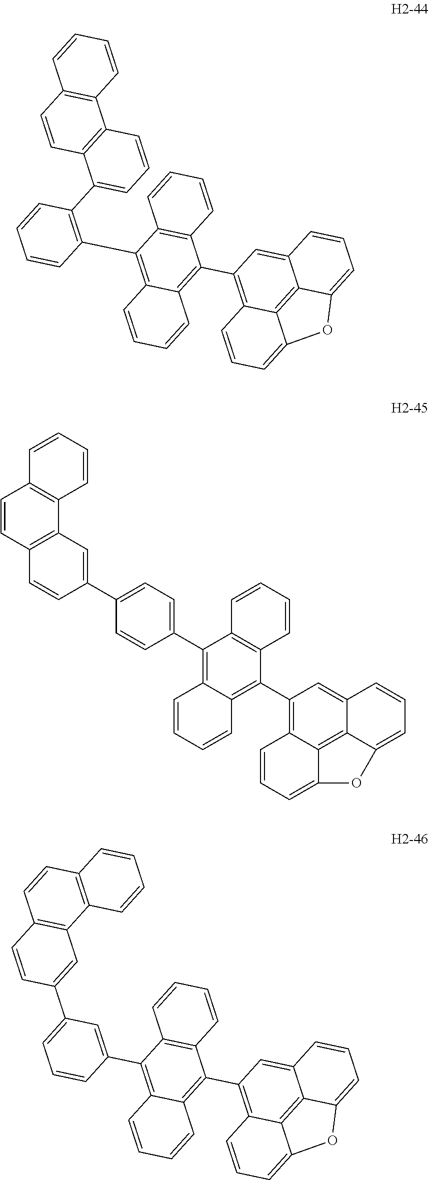 [SMILES: c1ccc(-c2cccc3c2ccc2ccccc23)c(-c2c3ccccc3c(-c3cc4cccc5oc6cccc3c6c45)c3ccccc23)c1 ; c1ccc2c(c1)ccc1ccc(-c3ccc(-c4c5ccccc5c(-c5cc6cccc7oc8cccc5c8c67)c5ccccc45)cc3)cc12 ; c1cc(-c2ccc3ccc4ccccc4c3c2)cc(-c2c3ccccc3c(-c3cc4cccc5oc6cccc3c6c45)c3ccccc23)c1] 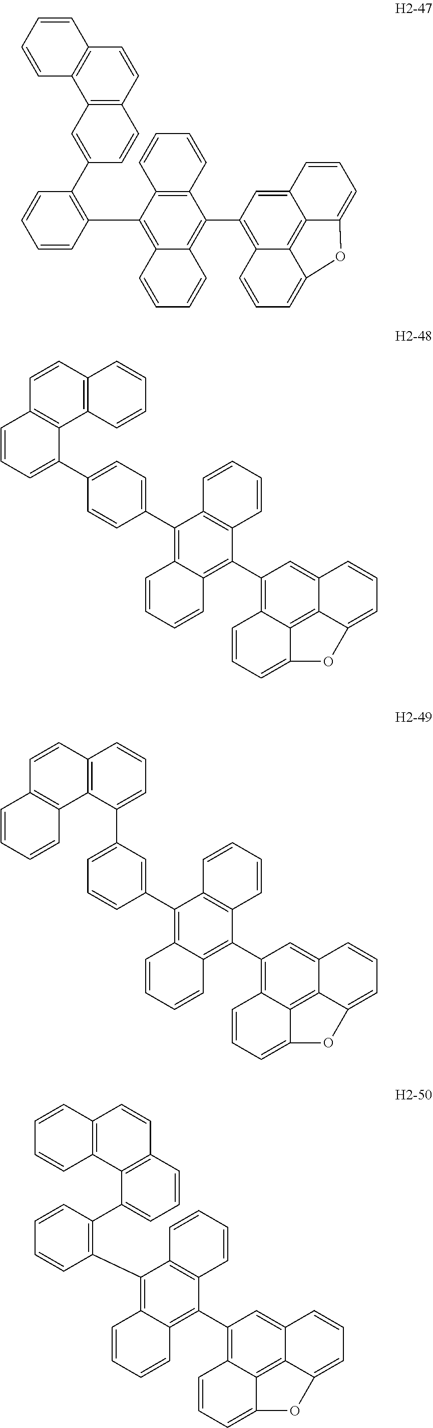 [SMILES: c1ccc(-c2c3ccccc3c(-c3cc4cccc5oc6cccc3c6c45)c3ccccc23)c(-c2ccc3ccc4ccccc4c3c2)c1 ; c1ccc2c(c1)ccc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3ccc4ccccc4c23)c1 ; c1ccc(-c2cccc3ccc4ccccc4c23)c(-c2c3ccccc3c(-c3cc4cccc5oc6cccc3c6c45)c3ccccc23)c1] 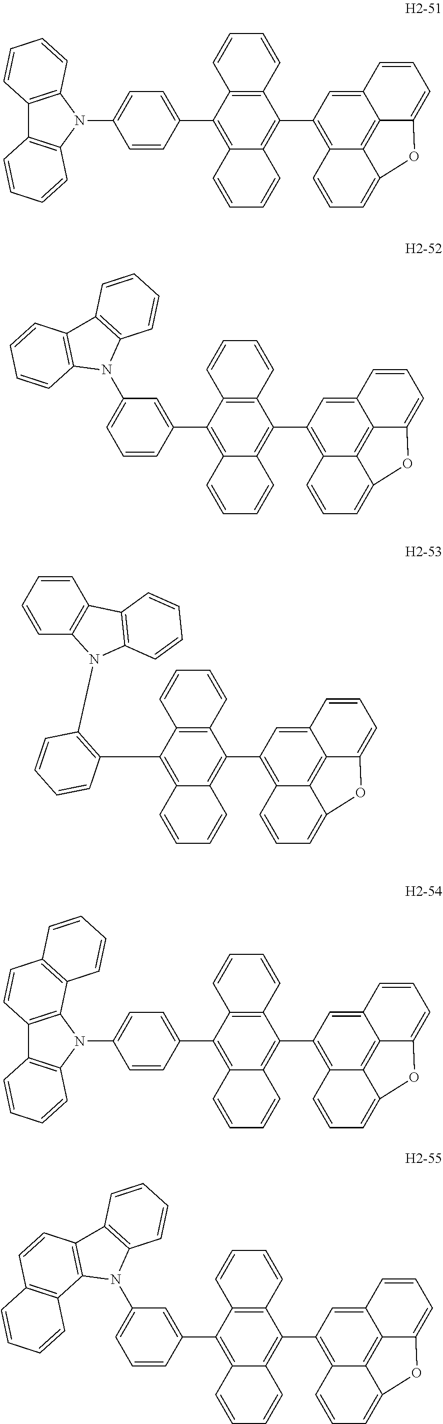 [SMILES: c1cc2cc(-c3c4ccccc4c(-c4ccc(-n5c6ccccc6c6ccccc65)cc4)c4ccccc34)c3cccc4oc(c1)c2c43 ; c1cc(-c2c3ccccc3c(-c3cc4cccc5oc6cccc3c6c45)c3ccccc23)cc(-n2c3ccccc3c3ccccc32)c1 ; c1ccc(-n2c3ccccc3c3ccccc32)c(-c2c3ccccc3c(-c3cc4cccc5oc6cccc3c6c45)c3ccccc23)c1 ; c1ccc2c(c1)ccc1c3ccccc3n(-c3ccc(-c4c5ccccc5c(-c5cc6cccc7oc8cccc5c8c67)c5ccccc45)cc3)c21 ; c1cc(-c2c3ccccc3c(-c3cc4cccc5oc6cccc3c6c45)c3ccccc23)cc(-n2c3ccccc3c3ccc4ccccc4c32)c1] 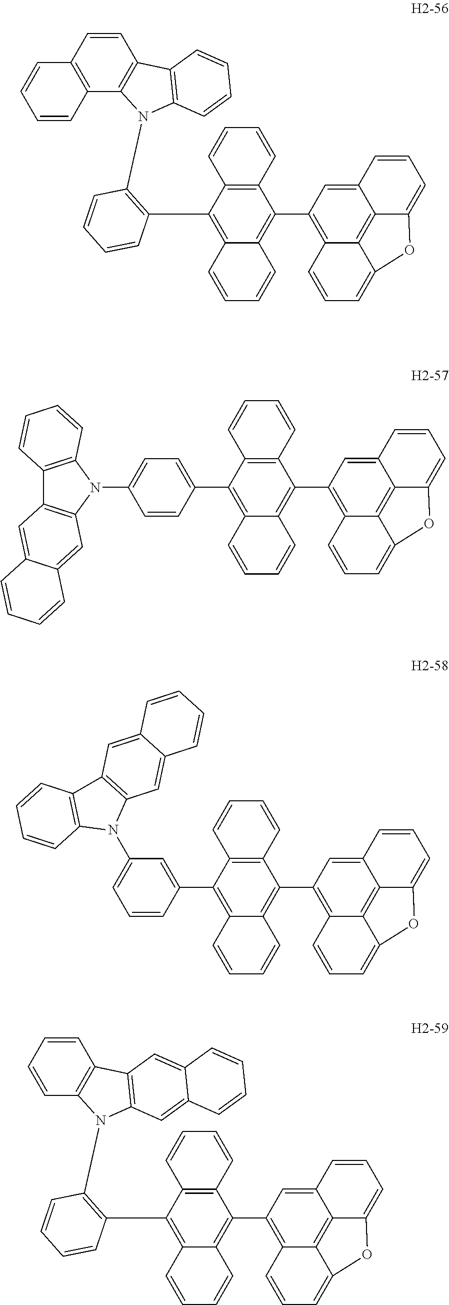 [SMILES: c1ccc(-n2c3ccccc3c3ccc4ccccc4c32)c(-c2c3ccccc3c(-c3cc4cccc5oc6cccc3c6c45)c3ccccc23)c1 ; c1ccc2cc3c(cc2c1)c1ccccc1n3-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3cc4ccccc4cc32)c1 ; c1ccc(-n2c3ccccc3c3cc4ccccc4cc32)c(-c2c3ccccc3c(-c3cc4cccc5oc6cccc3c6c45)c3ccccc23)c1] 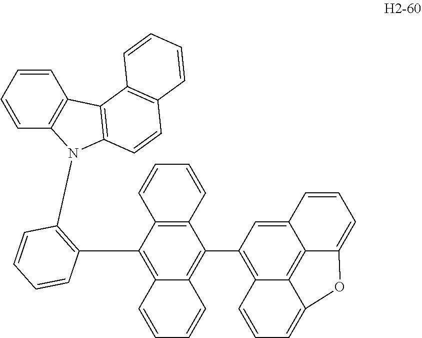 [SMILES: c1ccc(-n2c3ccccc3c3c4ccccc4ccc32)c(-c2c3ccccc3c(-c3cc4cccc5oc6cccc3c6c45)c3ccccc23)c1] 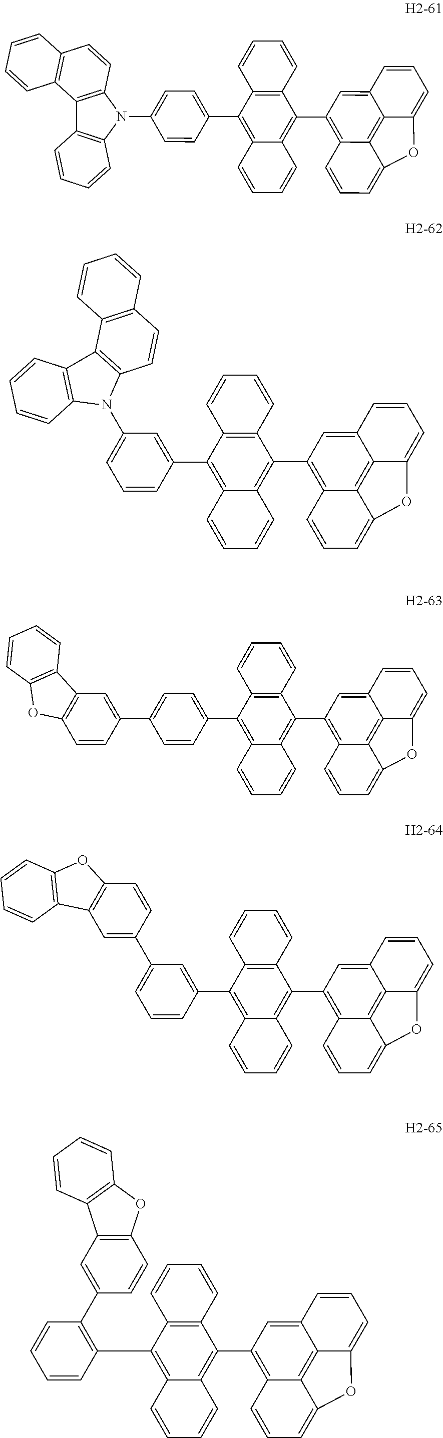 [SMILES: c1ccc2c(c1)ccc1c2c2ccccc2n1-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3c4ccccc4ccc32)c1 ; c1ccc2c(c1)oc1ccc(-c3ccc(-c4c5ccccc5c(-c5cc6cccc7oc8cccc5c8c67)c5ccccc45)cc3)cc12 ; c1cc(-c2ccc3oc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3oc4ccccc4c3c2)c1] 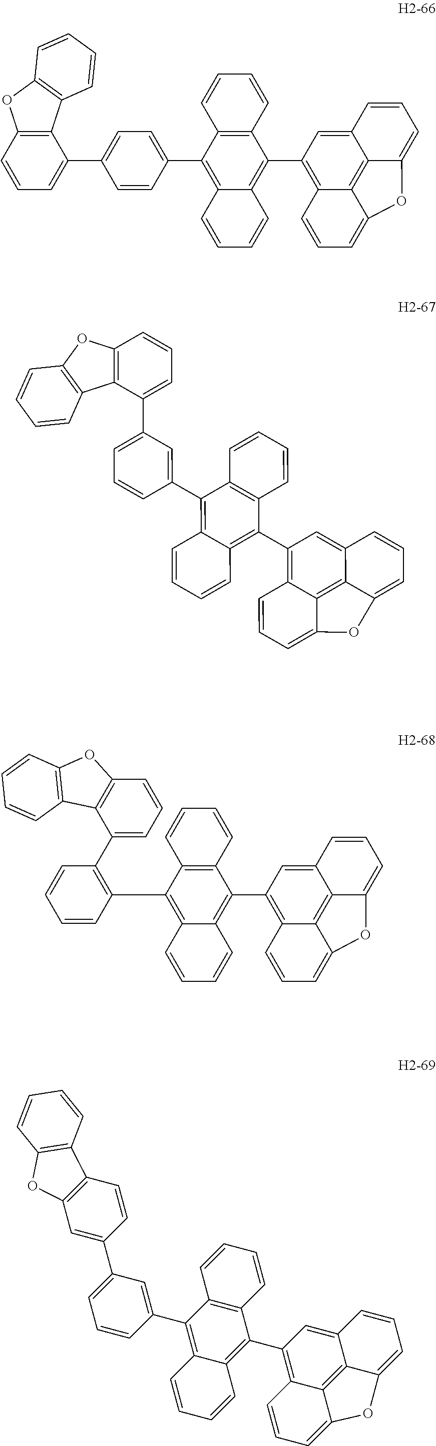 [SMILES: c1ccc2c(c1)oc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3oc4ccccc4c23)c1 ; c1ccc(-c2cccc3oc4ccccc4c23)c(-c2c3ccccc3c(-c3cc4cccc5oc6cccc3c6c45)c3ccccc23)c1 ; c1cc(-c2ccc3c(c2)oc2ccccc23)cc(-c2c3ccccc3c(-c3cc4cccc5oc6cccc3c6c45)c3ccccc23)c1] 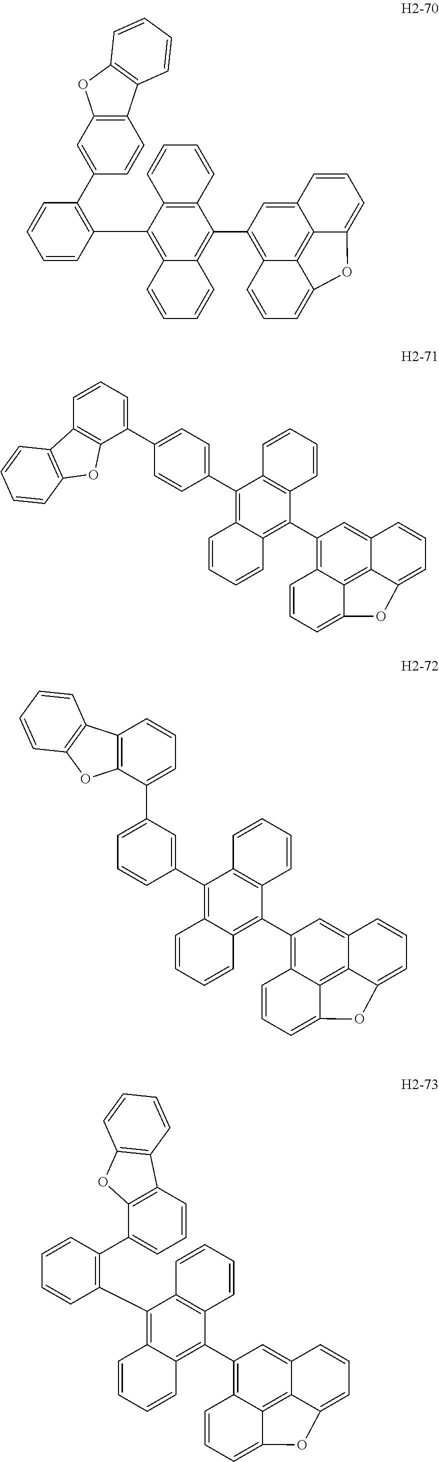 [SMILES: c1ccc(-c2c3ccccc3c(-c3cc4cccc5oc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5oc6cccc3c6c45)c3ccccc23)c1] 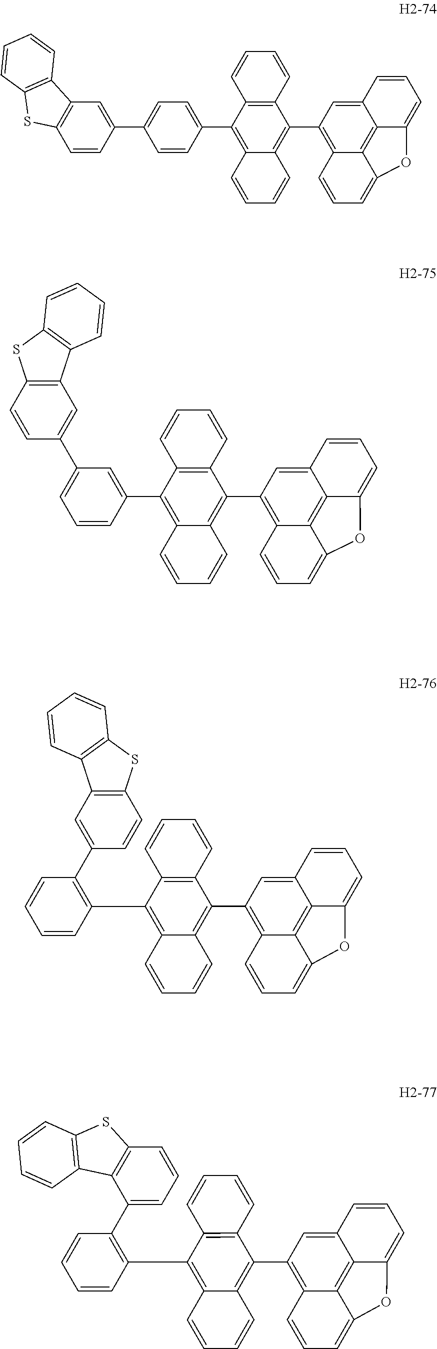 [SMILES: c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1] 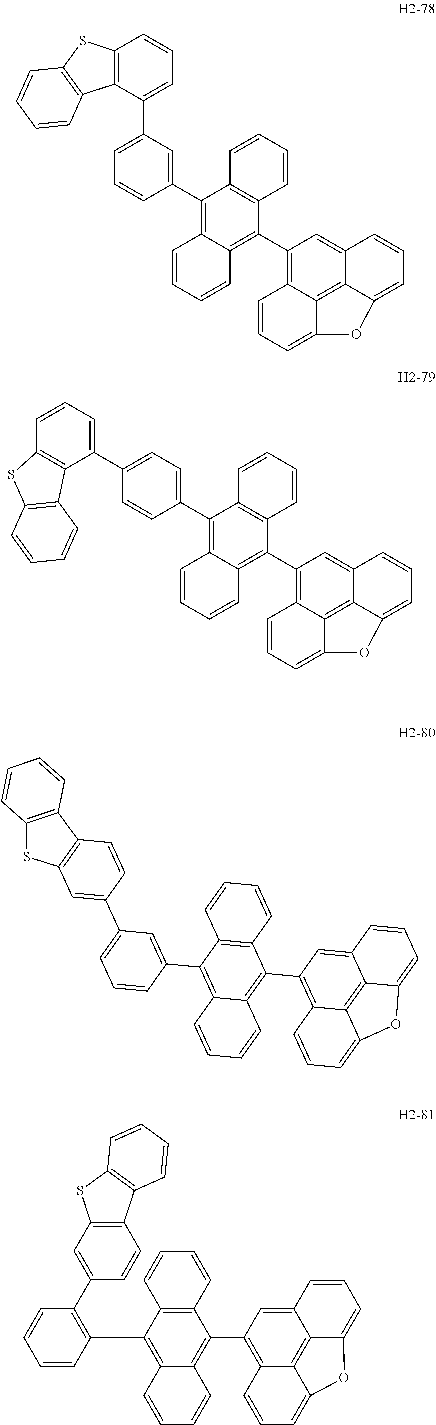 [SMILES: c1cc(-c2c3ccccc3c(-c3cc4cccc5oc6cccc3c6c45)c3ccccc23)cc(-c2cccc3sc4ccccc4c23)c1 ; c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1] 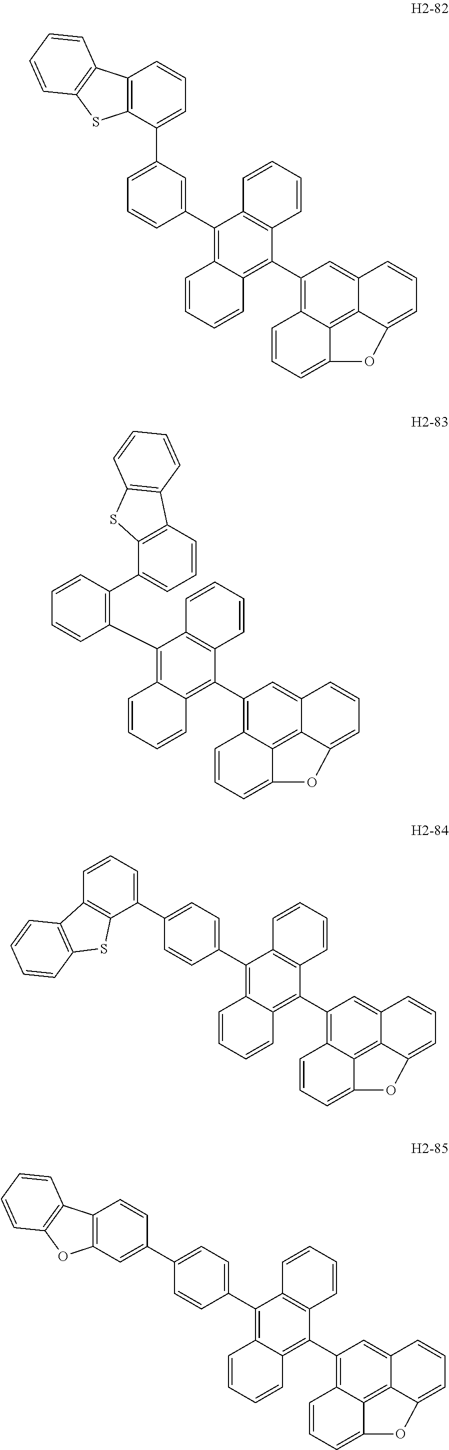 [SMILES: c1cc(-c2c3ccccc3c(-c3cc4cccc5oc6cccc3c6c45)c3ccccc23)cc(-c2cccc3c2sc2ccccc23)c1 ; c1ccc(-c2cccc3c2sc2ccccc23)c(-c2c3ccccc3c(-c3cc4cccc5oc6cccc3c6c45)c3ccccc23)c1 ; c1cc2cc(-c3c4ccccc4c(-c4ccc(-c5cccc6c5sc5ccccc56)cc4)c4ccccc34)c3cccc4oc(c1)c2c43 ; c1ccc2c(c1)oc1cc(-c3ccc(-c4c5ccccc5c(-c5cc6cccc7oc8cccc5c8c67)c5ccccc45)cc3)ccc12] 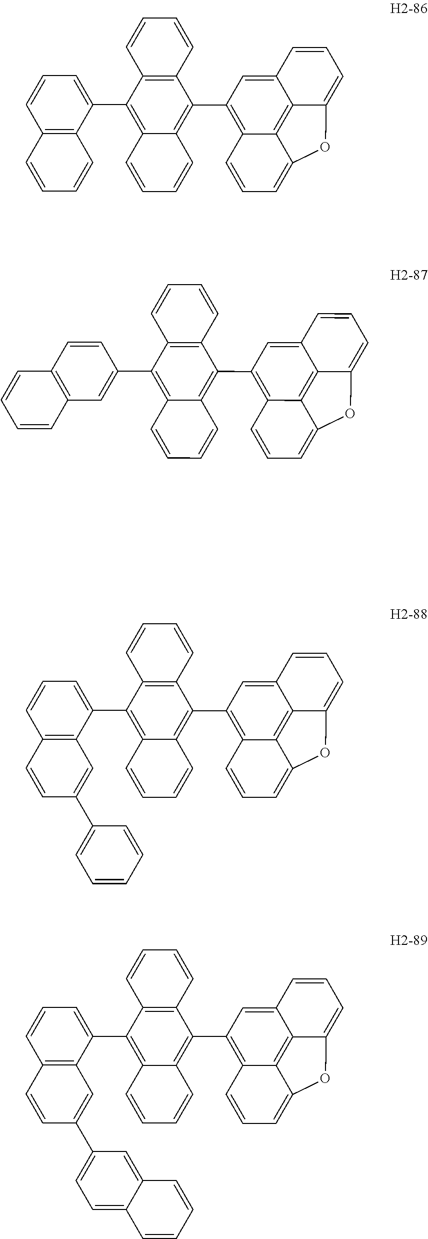 [SMILES: c1ccc2c(-c3c4ccccc4c(-c4cc5cccc6oc7cccc4c7c56)c4ccccc34)cccc2c1 ; c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1] 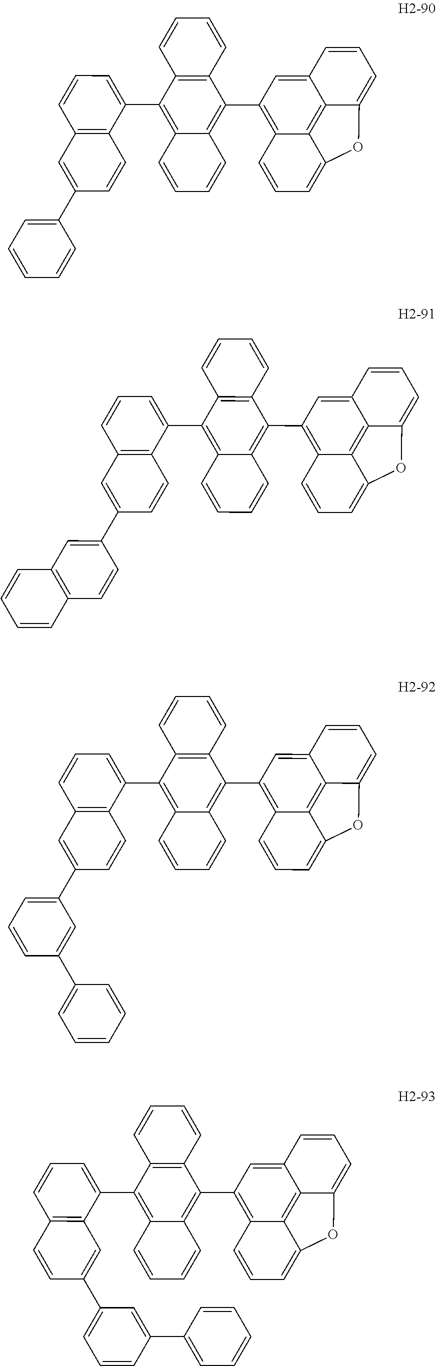 [SMILES: c1ccc(-c2ccc3c(-c4c5ccccc5c(-c5cc6cccc7oc8cccc5c8c67)c5ccccc45)cccc3c2)cc1 ; c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1] 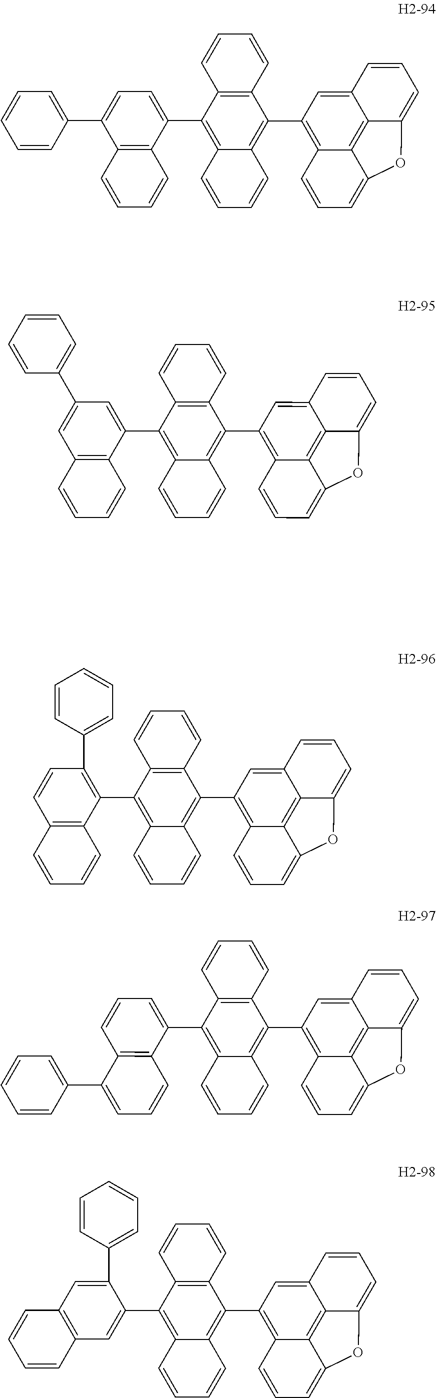 [SMILES: c1ccc(-c2ccc(-c3c4ccccc4c(-c4cc5cccc6oc7cccc4c7c56)c4ccccc34)c3ccccc23)cc1 ; c1ccc(-c2cc(-c3c4ccccc4c(-c4cc5cccc6oc7cccc4c7c56)c4ccccc34)c3ccccc3c2)cc1 ; c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1] 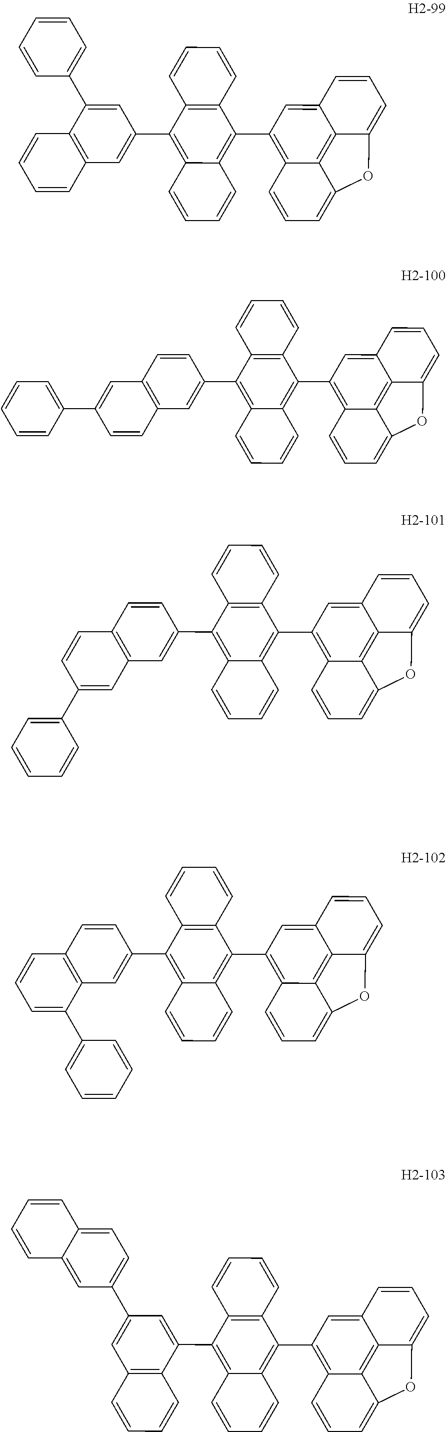 [SMILES: c1ccc(-c2cc(-c3c4ccccc4c(-c4cc5cccc6oc7cccc4c7c56)c4ccccc34)cc3ccccc23)cc1 ; c1ccc(-c2ccc3cc(-c4c5ccccc5c(-c5cc6cccc7oc8cccc5c8c67)c5ccccc45)ccc3c2)cc1 ; c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2cc(-c3cc(-c4c5ccccc5c(-c5cc6cccc7oc8cccc5c8c67)c5ccccc45)c4ccccc4c3)ccc2c1] 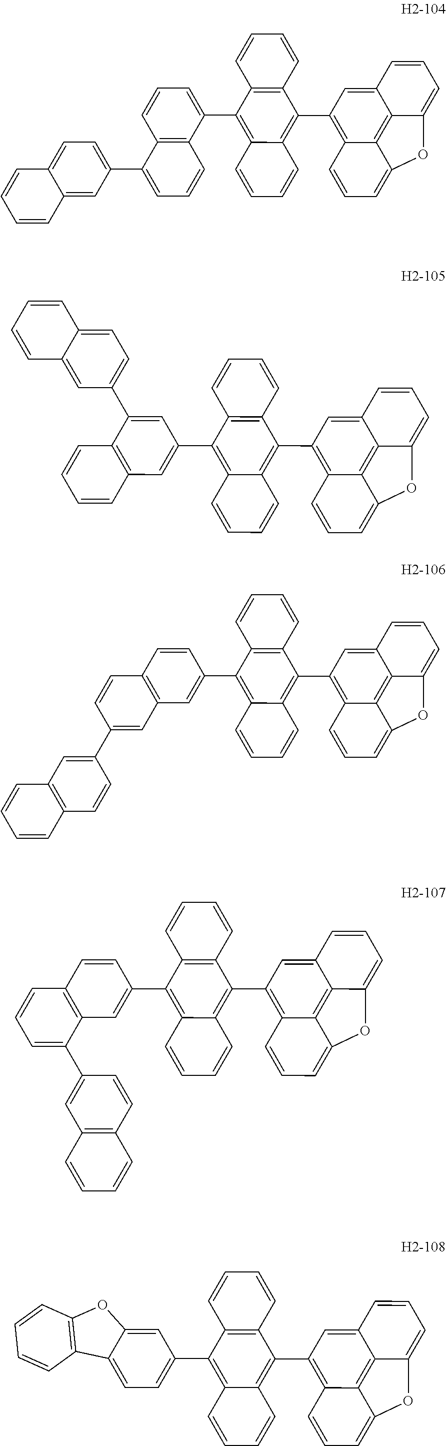 [SMILES: c1ccc2cc(-c3cccc4c(-c5c6ccccc6c(-c6cc7cccc8oc9cccc6c9c78)c6ccccc56)cccc34)ccc2c1 ; c1ccc2cc(-c3cc(-c4c5ccccc5c(-c5cc6cccc7oc8cccc5c8c67)c5ccccc45)cc4ccccc34)ccc2c1 ; c1ccc2cc(-c3ccc4ccc(-c5c6ccccc6c(-c6cc7cccc8oc9cccc6c9c78)c6ccccc56)cc4c3)ccc2c1 ; c1ccc2cc(-c3cccc4ccc(-c5c6ccccc6c(-c6cc7cccc8oc9cccc6c9c78)c6ccccc56)cc34)ccc2c1 ; c1ccc2c(c1)oc1cc(-c3c4ccccc4c(-c4cc5cccc6oc7cccc4c7c56)c4ccccc34)ccc12] 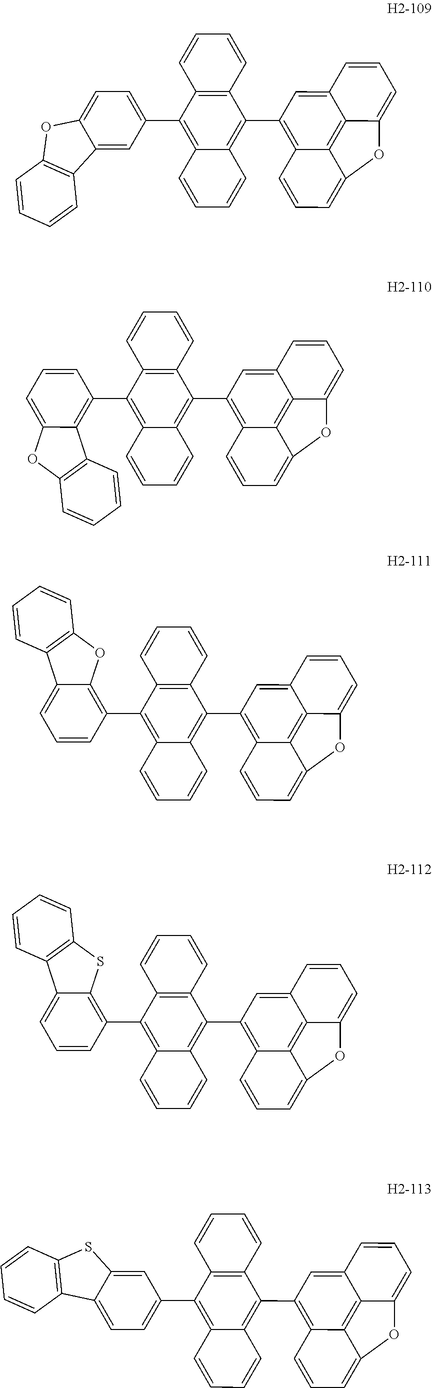 [SMILES: c1ccc2c(c1)oc1ccc(-c3c4ccccc4c(-c4cc5cccc6oc7cccc4c7c56)c4ccccc34)cc12 ; c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43] 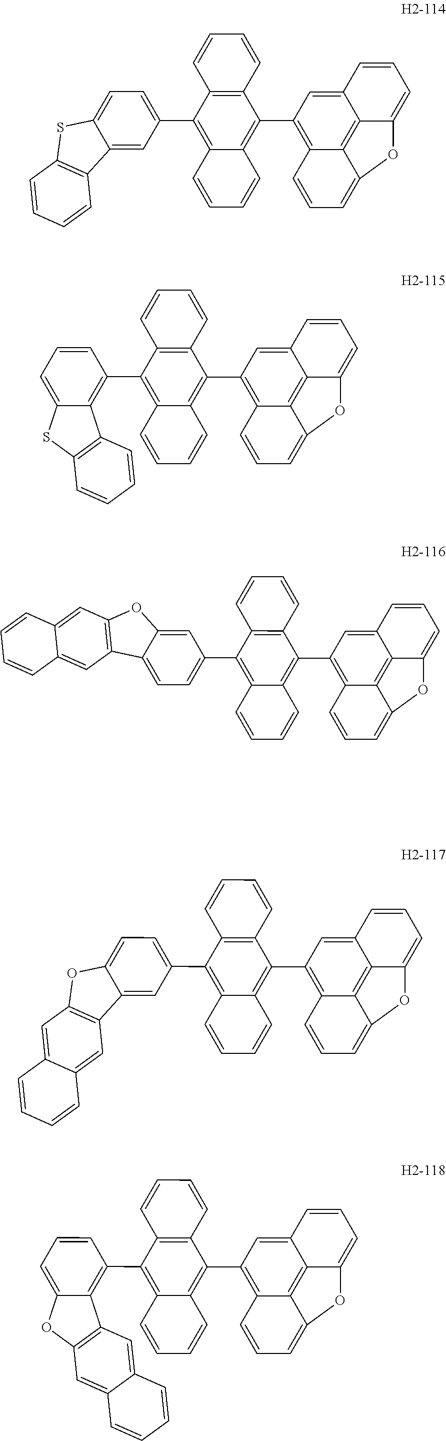 [SMILES: c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43 ; c1ccc2cc3c(cc2c1)oc1cc(-c2c4ccccc4c(-c4cc5cccc6oc7cccc4c7c56)c4ccccc24)ccc13 ; c1ccc2cc3c(cc2c1)oc1ccc(-c2c4ccccc4c(-c4cc5cccc6oc7cccc4c7c56)c4ccccc24)cc13 ; c1ccc2cc3c(cc2c1)oc1cccc(-c2c4ccccc4c(-c4cc5cccc6oc7cccc4c7c56)c4ccccc24)c13] 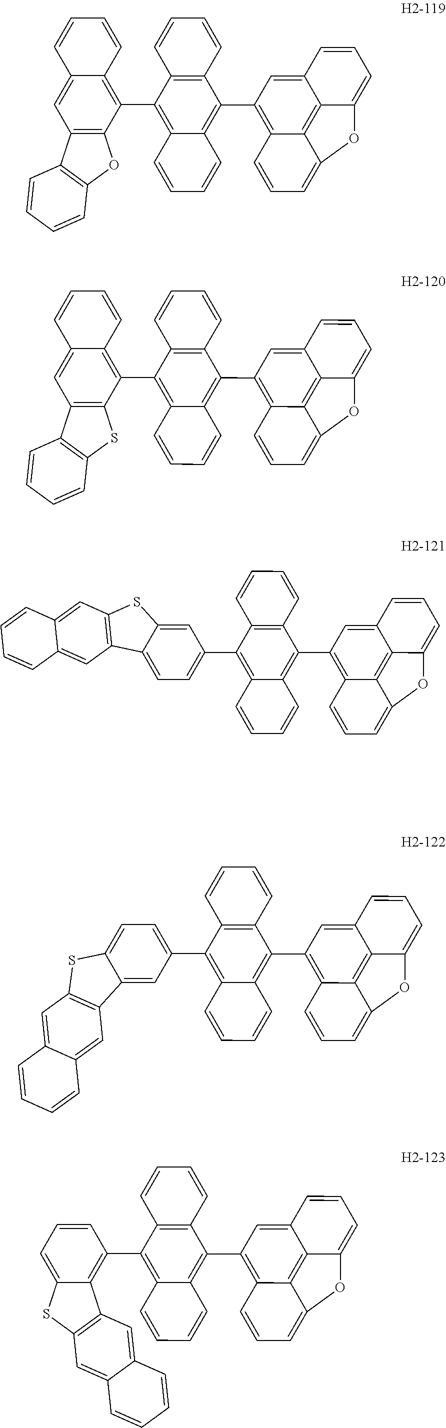 [SMILES: c1ccc2c(-c3c4ccccc4c(-c4cc5cccc6oc7cccc4c7c56)c4ccccc34)c3oc4ccccc4c3cc2c1 ; c1ccc2c(-c3c4ccccc4c(-c4cc5cccc6oc7cccc4c7c56)c4ccccc34)c3sc4ccccc4c3cc2c1 ; c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13] 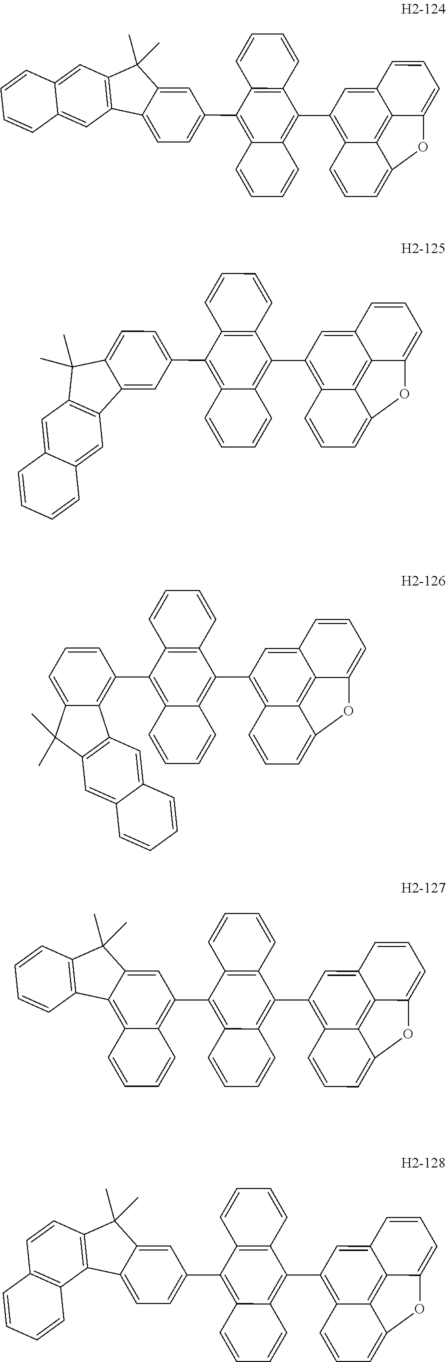 [SMILES: CC1(C)c2cc(-c3c4ccccc4c(-c4cc5cccc6oc7cccc4c7c56)c4ccccc34)ccc2-c2cc3ccccc3cc21 ; CC1(C)c2ccc(-c3c4ccccc4c(-c4cc5cccc6oc7cccc4c7c56)c4ccccc34)cc2-c2cc3ccccc3cc21 ; CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21] 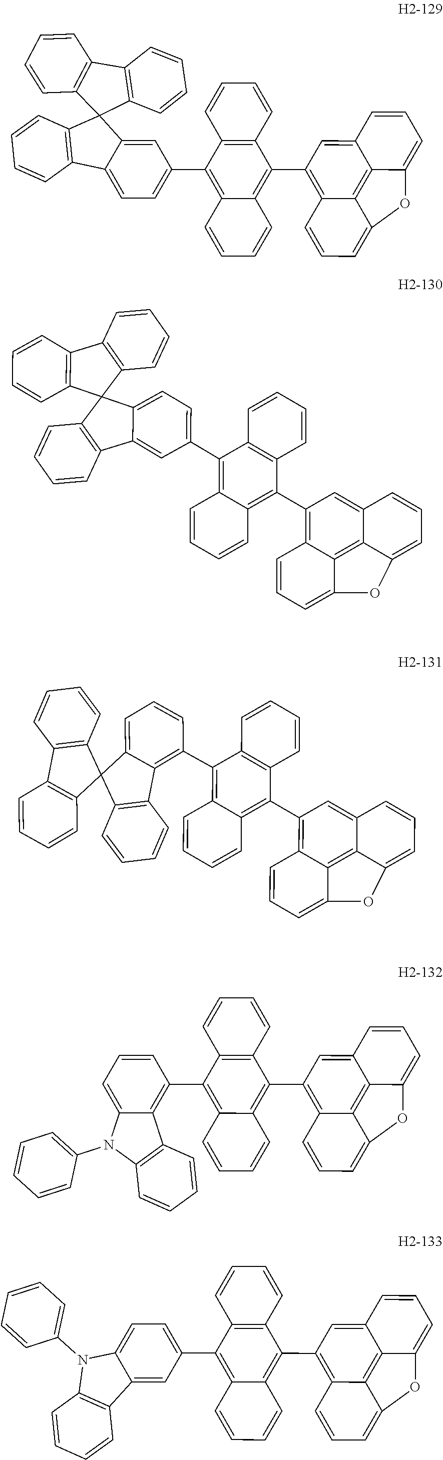 [SMILES: c1ccc2c(c1)-c1ccccc1C21c2ccccc2-c2ccc(-c3c4ccccc4c(-c4cc5cccc6oc7cccc4c7c56)c4ccccc34)cc21 ; c1ccc2c(c1)-c1ccccc1C21c2ccccc2-c2cc(-c3c4ccccc4c(-c4cc5cccc6oc7cccc4c7c56)c4ccccc34)ccc21 ; c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1 ; c1ccc(-n2c3ccccc3c3cc(-c4c5ccccc5c(-c5cc6cccc7oc8cccc5c8c67)c5ccccc45)ccc32)cc1] 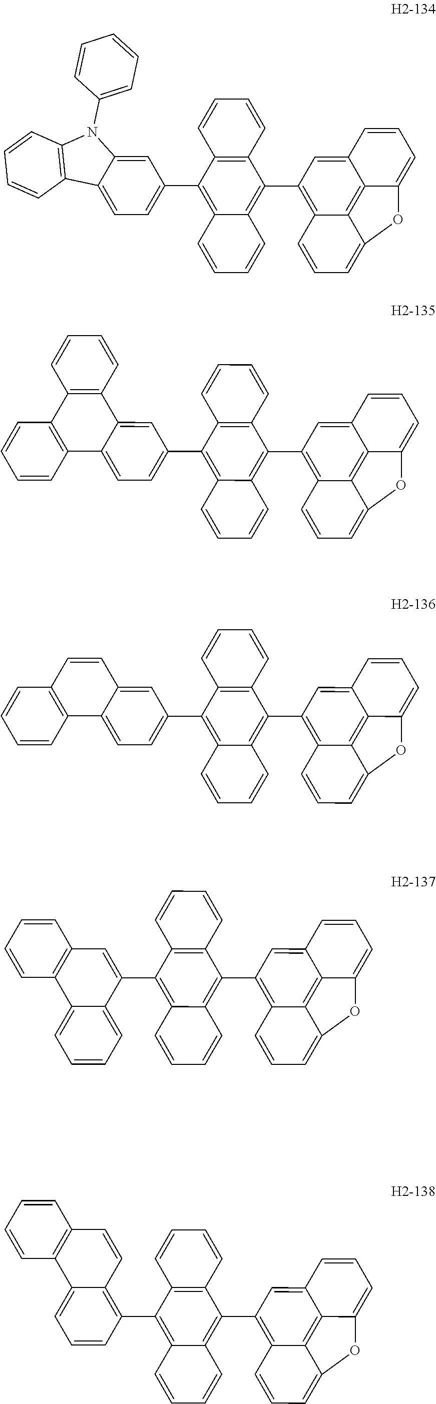 [SMILES: c1ccc(-n2c3ccccc3c3ccc(-c4c5ccccc5c(-c5cc6cccc7oc8cccc5c8c67)c5ccccc45)cc32)cc1 ; c1cc2cc(-c3c4ccccc4c(-c4ccc5c6ccccc6c6ccccc6c5c4)c4ccccc34)c3cccc4oc(c1)c2c43 ; c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; c1ccc2c(c1)ccc1c(-c3c4ccccc4c(-c4cc5cccc6oc7cccc4c7c56)c4ccccc34)cccc12] 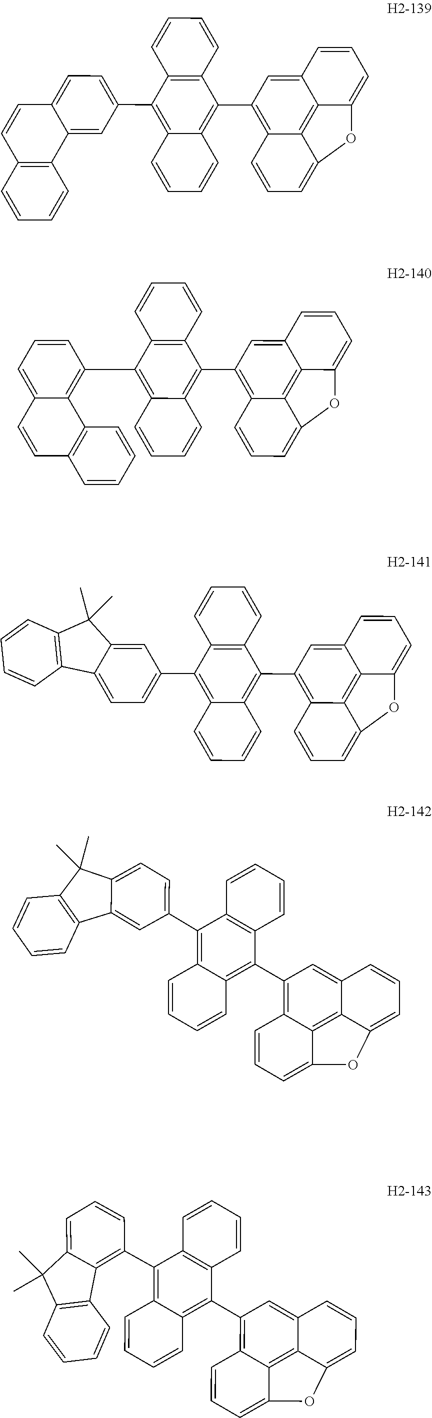 [SMILES: c1ccc2c(c1)ccc1ccc(-c3c4ccccc4c(-c4cc5cccc6oc7cccc4c7c56)c4ccccc34)cc12 ; c1ccc2c(c1)ccc1cccc(-c3c4ccccc4c(-c4cc5cccc6oc7cccc4c7c56)c4ccccc34)c12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21] 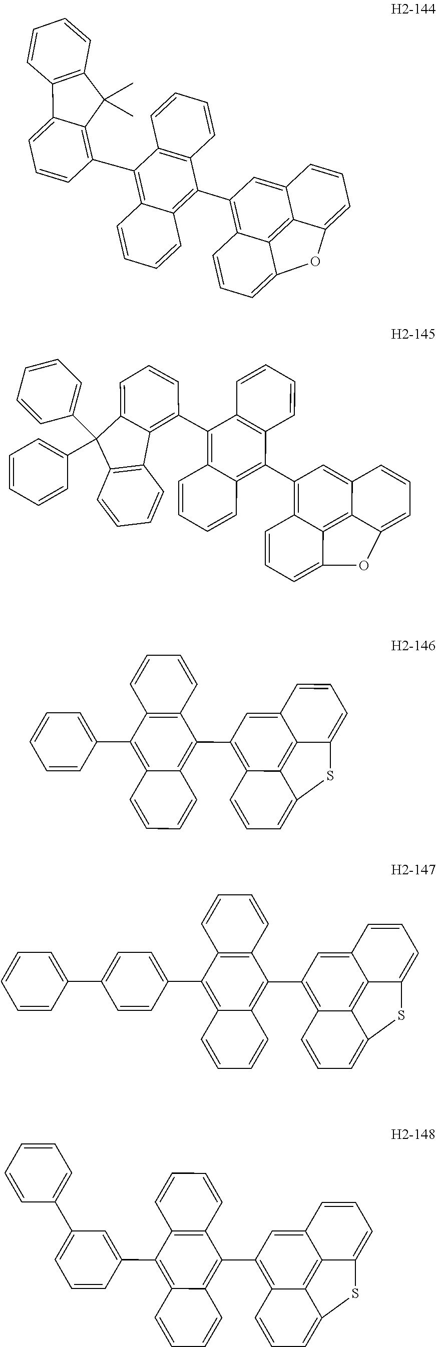 [SMILES: CC1(C)c2ccccc2-c2cccc(-c3c4ccccc4c(-c4cc5cccc6oc7cccc4c7c56)c4ccccc34)c21 ; c1ccc(C2(c3ccccc3)c3ccccc3-c3c(-c4c5ccccc5c(-c5cc6cccc7oc8cccc5c8c67)c5ccccc45)cccc32)cc1 ; c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1] 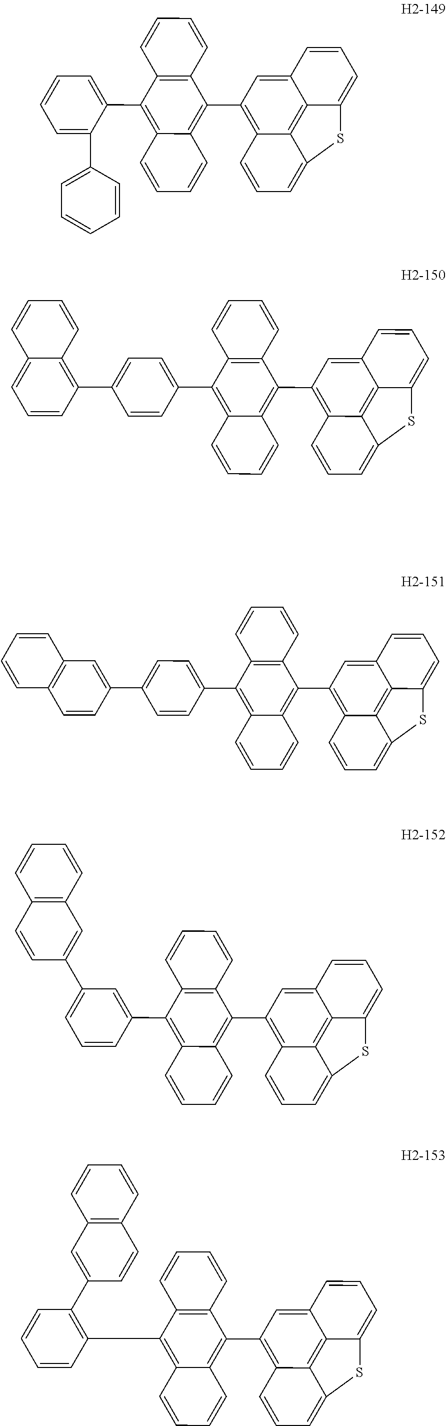 [SMILES: c1ccc(-c2ccccc2-c2c3ccccc3c(-c3cc4cccc5sc6cccc3c6c45)c3ccccc23)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1 ; c1cc(-c2ccc3ccccc3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3ccccc3c2)c1] 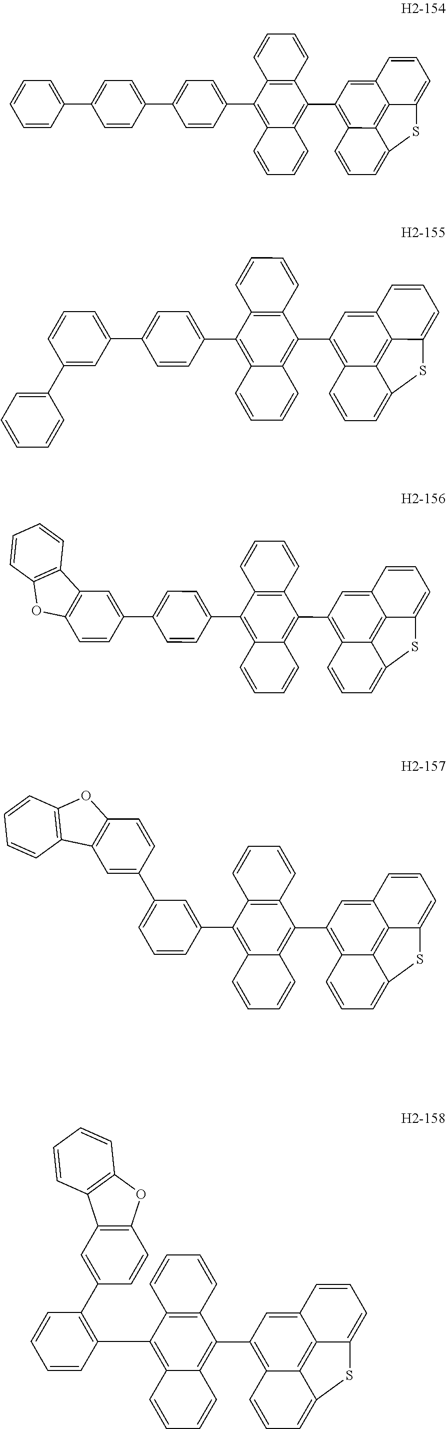 [SMILES: c1ccc(-c2ccc(-c3ccc(-c4c5ccccc5c(-c5cc6cccc7sc8cccc5c8c67)c5ccccc45)cc3)cc2)cc1 ; c1ccc(-c2cccc(-c3ccc(-c4c5ccccc5c(-c5cc6cccc7sc8cccc5c8c67)c5ccccc45)cc3)c2)cc1 ; c1ccc2c(c1)oc1ccc(-c3ccc(-c4c5ccccc5c(-c5cc6cccc7sc8cccc5c8c67)c5ccccc45)cc3)cc12 ; c1cc(-c2ccc3oc4ccccc4c3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3oc4ccccc4c3c2)c1] 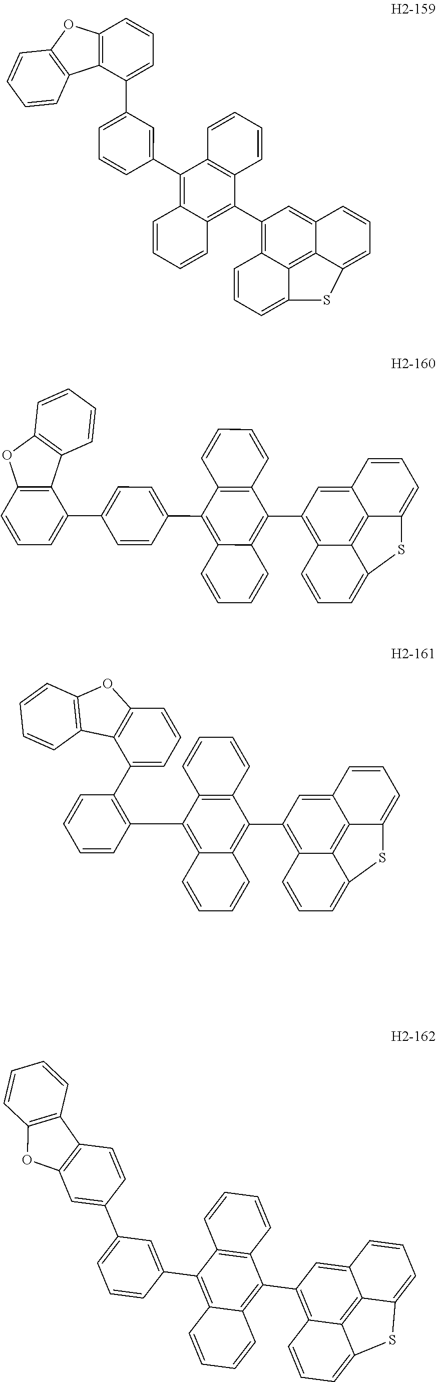 [SMILES: c1cc(-c2c3ccccc3c(-c3cc4cccc5sc6cccc3c6c45)c3ccccc23)cc(-c2cccc3oc4ccccc4c23)c1 ; c1ccc2c(c1)oc1cccc(-c3ccc(-c4c5ccccc5c(-c5cc6cccc7sc8cccc5c8c67)c5ccccc45)cc3)c12 ; c1ccc(-c2cccc3oc4ccccc4c23)c(-c2c3ccccc3c(-c3cc4cccc5sc6cccc3c6c45)c3ccccc23)c1 ; c1cc(-c2ccc3c(c2)oc2ccccc23)cc(-c2c3ccccc3c(-c3cc4cccc5sc6cccc3c6c45)c3ccccc23)c1] 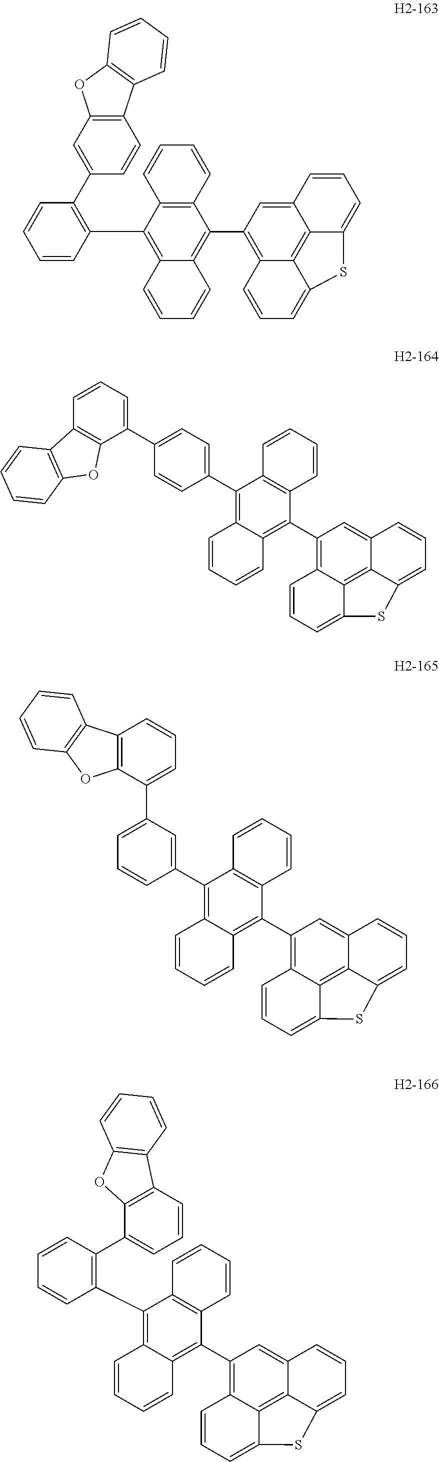 [SMILES: c1ccc(-c2c3ccccc3c(-c3cc4cccc5sc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7sc8cccc5c8c67)c5ccccc45)cc3)cccc12 ; c1cc(-c2c3ccccc3c(-c3cc4cccc5sc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5sc6cccc3c6c45)c3ccccc23)c1] 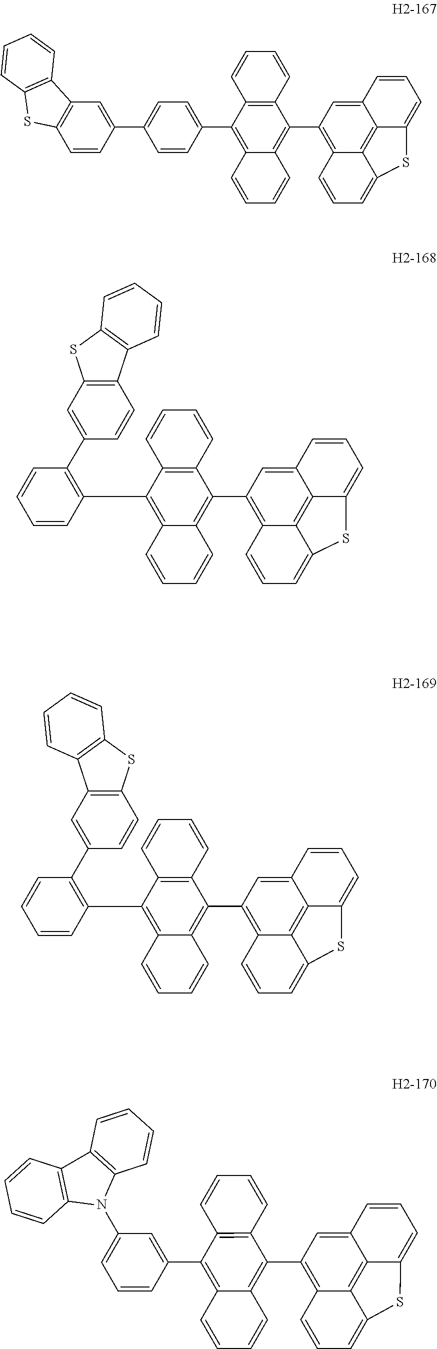 [SMILES: c1ccc2c(c1)sc1ccc(-c3ccc(-c4c5ccccc5c(-c5cc6cccc7sc8cccc5c8c67)c5ccccc45)cc3)cc12 ; c1ccc(-c2c3ccccc3c(-c3cc4cccc5sc6cccc3c6c45)c3ccccc23)c(-c2ccc3c(c2)sc2ccccc23)c1 ; c1ccc(-c2c3ccccc3c(-c3cc4cccc5sc6cccc3c6c45)c3ccccc23)c(-c2ccc3sc4ccccc4c3c2)c1 ; c1cc(-c2c3ccccc3c(-c3cc4cccc5sc6cccc3c6c45)c3ccccc23)cc(-n2c3ccccc3c3ccccc32)c1] 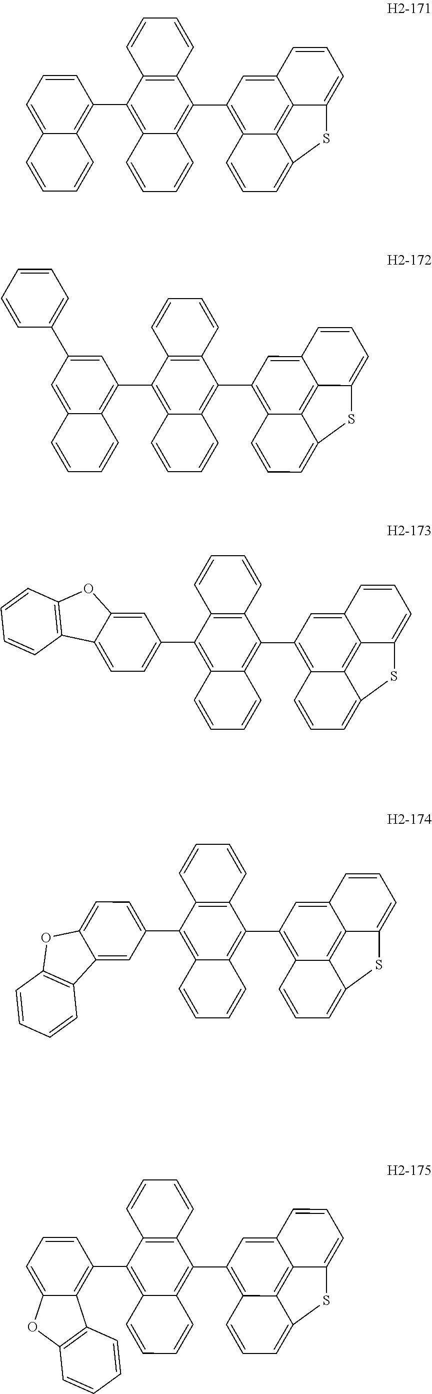 [SMILES: c1ccc2c(-c3c4ccccc4c(-c4cc5cccc6sc7cccc4c7c56)c4ccccc34)cccc2c1 ; c1ccc(-c2cc(-c3c4ccccc4c(-c4cc5cccc6sc7cccc4c7c56)c4ccccc34)c3ccccc3c2)cc1 ; c1ccc2c(c1)oc1cc(-c3c4ccccc4c(-c4cc5cccc6sc7cccc4c7c56)c4ccccc34)ccc12 ; c1ccc2c(c1)oc1ccc(-c3c4ccccc4c(-c4cc5cccc6sc7cccc4c7c56)c4ccccc34)cc12 ; c1ccc2c(c1)oc1cccc(-c3c4ccccc4c(-c4cc5cccc6sc7cccc4c7c56)c4ccccc34)c12] 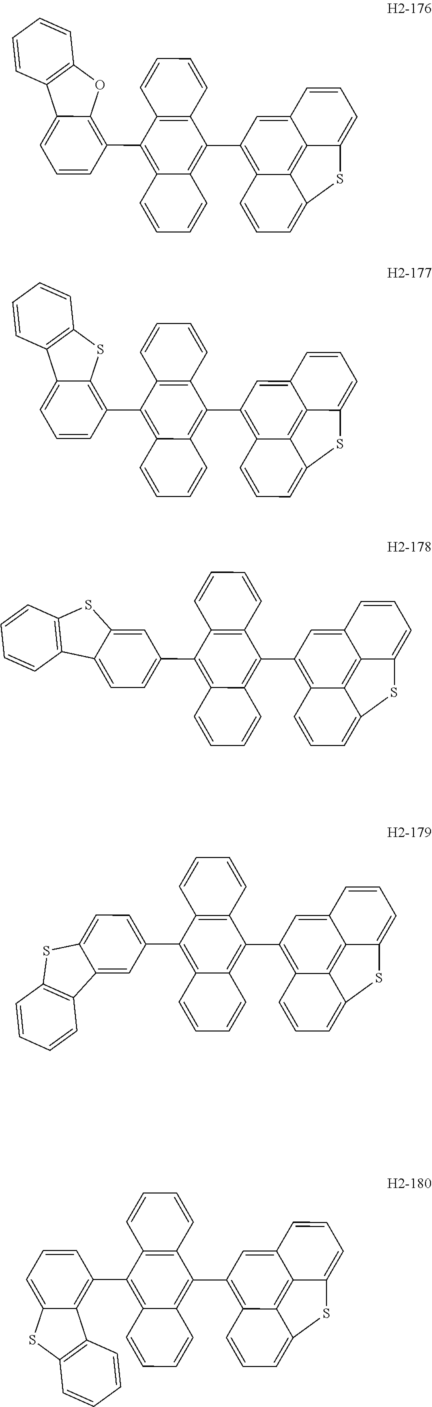 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12] 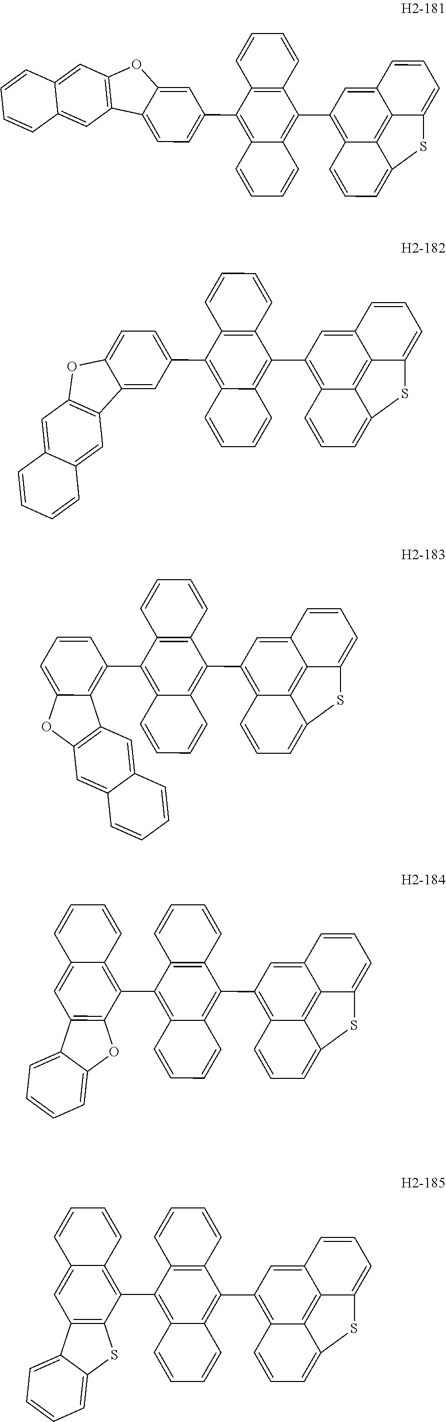 [SMILES: c1ccc2cc3c(cc2c1)oc1cc(-c2c4ccccc4c(-c4cc5cccc6sc7cccc4c7c56)c4ccccc24)ccc13 ; c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1 ; c1ccc2c(-c3c4ccccc4c(-c4cc5cccc6sc7cccc4c7c56)c4ccccc34)c3sc4ccccc4c3cc2c1] 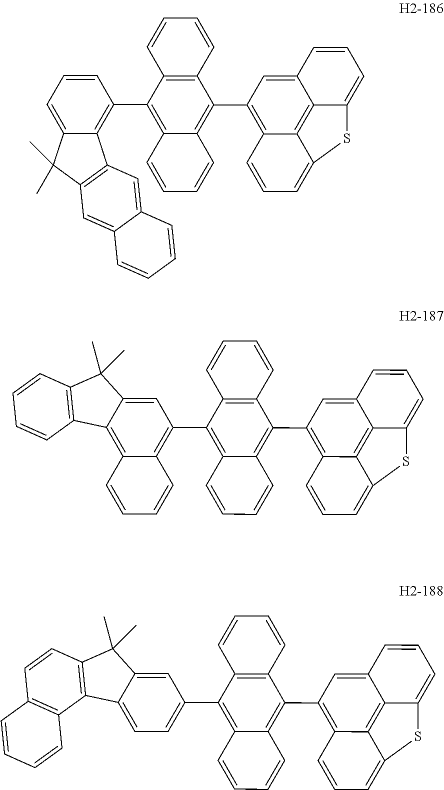 [SMILES: CC1(C)c2cc3ccccc3cc2-c2c(-c3c4ccccc4c(-c4cc5cccc6sc7cccc4c7c56)c4ccccc34)cccc21 ; CC1(C)c2ccccc2-c2c1cc(-c1c3ccccc3c(-c3cc4cccc5sc6cccc3c6c45)c3ccccc13)c1ccccc21 ; CC1(C)c2cc(-c3c4ccccc4c(-c4cc5cccc6sc7cccc4c7c56)c4ccccc34)ccc2-c2c1ccc1ccccc21] 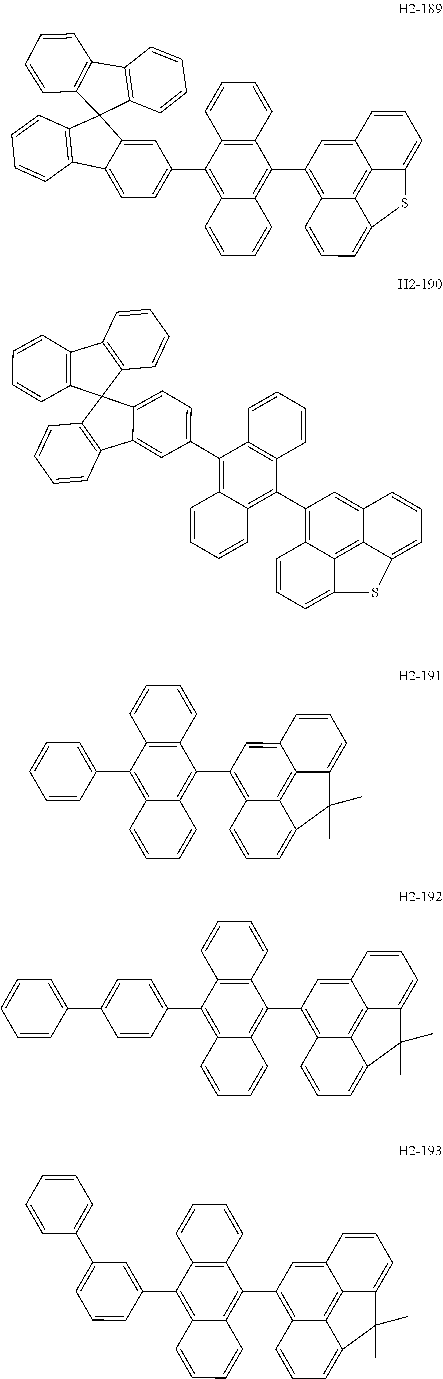 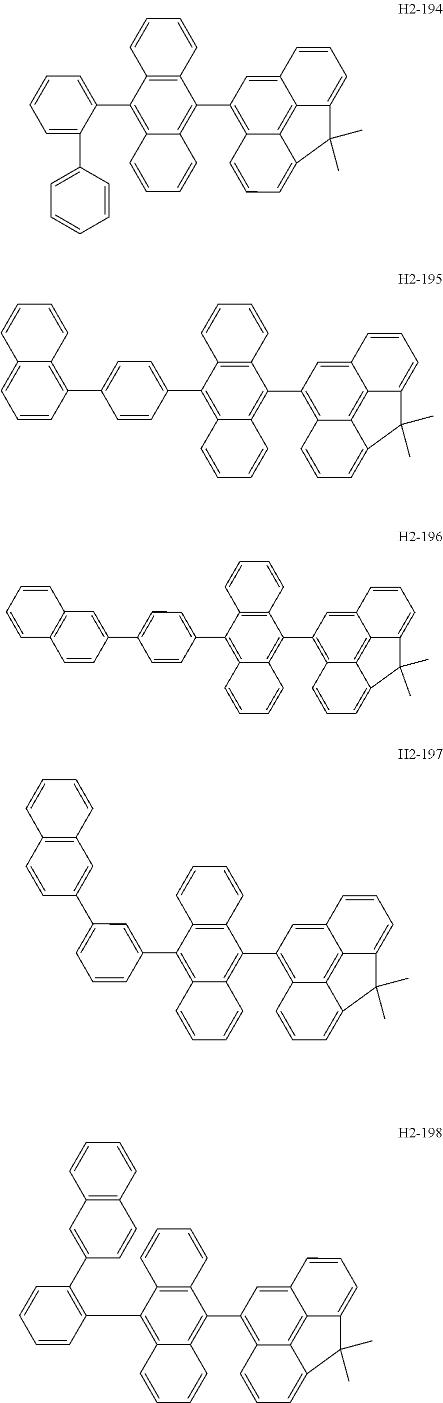 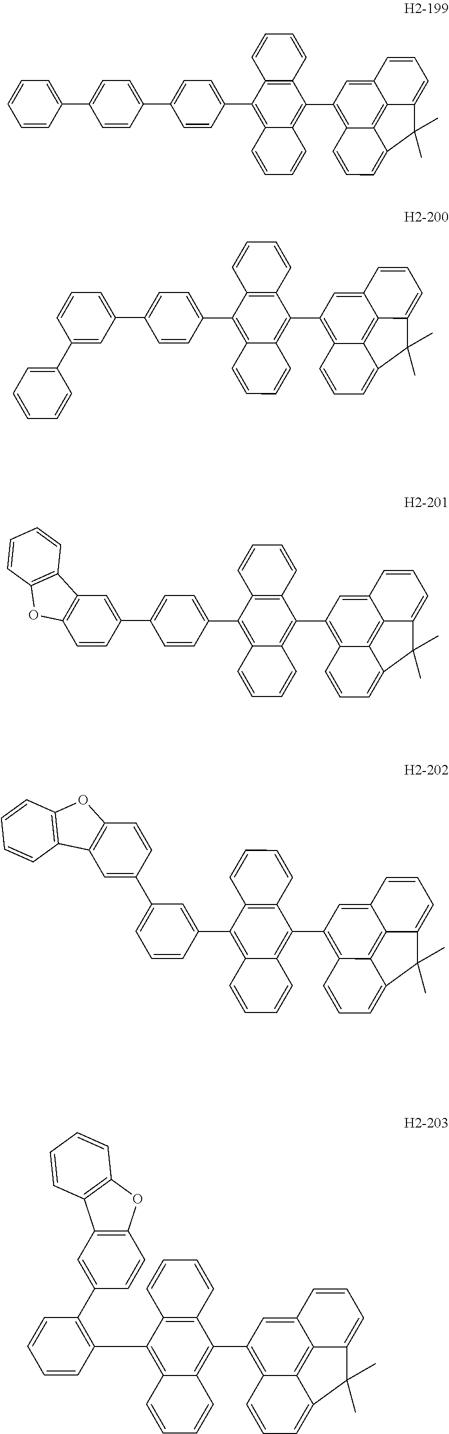 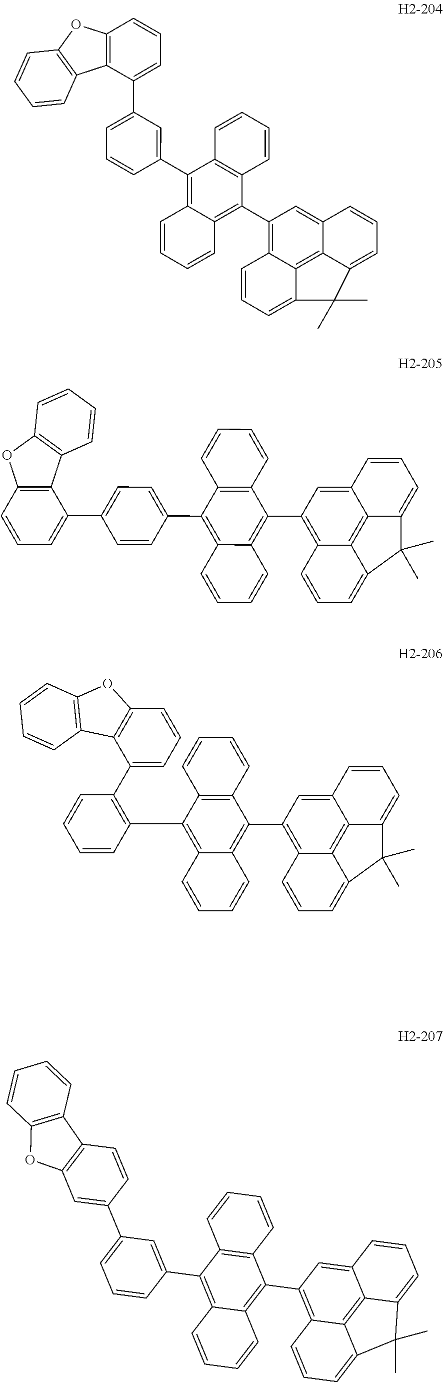 [SMILES: CC1(C)c2cccc3cc(-c4c5ccccc5c(-c5cccc(-c6cccc7oc8ccccc8c67)c5)c5ccccc45)c4cccc1c4c23 ; CC1(C)c2cccc3cc(-c4c5ccccc5c(-c5ccc(-c6cccc7oc8ccccc8c67)cc5)c5ccccc45)c4cccc1c4c23 ; CC1(C)c2cccc3cc(-c4c5ccccc5c(-c5ccccc5-c5cccc6oc7ccccc7c56)c5ccccc45)c4cccc1c4c23 ; CC1(C)c2cccc3cc(-c4c5ccccc5c(-c5cccc(-c6ccc7c(c6)oc6ccccc67)c5)c5ccccc45)c4cccc1c4c23] 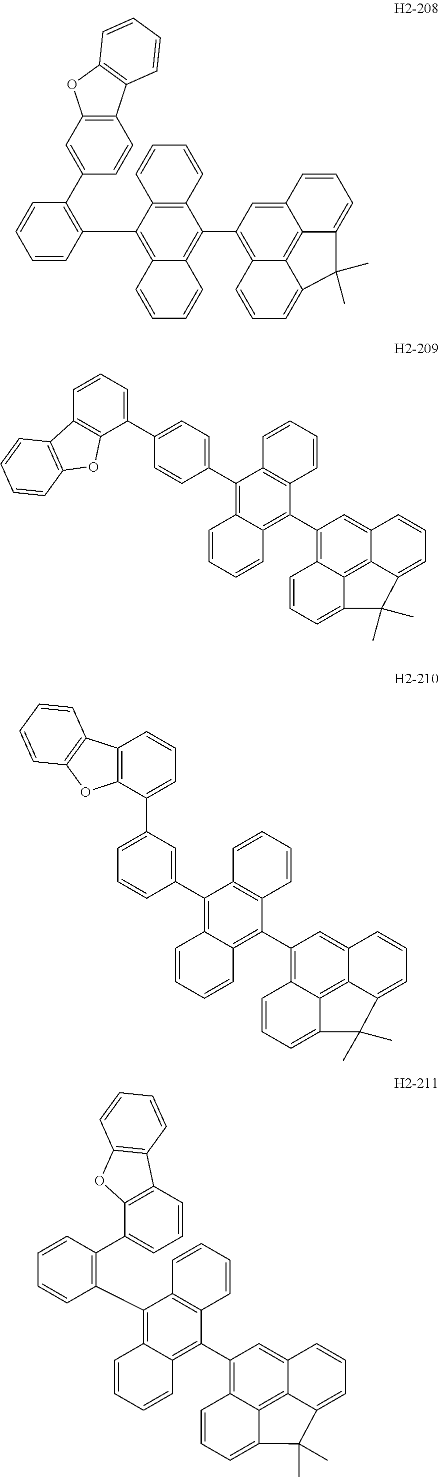 [SMILES: CC1(C)c2cccc3cc(-c4c5ccccc5c(-c5ccccc5-c5ccc6c(c5)oc5ccccc56)c5ccccc45)c4cccc1c4c23 ; CC1(C)c2cccc3cc(-c4c5ccccc5c(-c5ccc(-c6cccc7c6oc6ccccc67)cc5)c5ccccc45)c4cccc1c4c23 ; CC1(C)c2cccc3cc(-c4c5ccccc5c(-c5cccc(-c6cccc7c6oc6ccccc67)c5)c5ccccc45)c4cccc1c4c23 ; CC1(C)c2cccc3cc(-c4c5ccccc5c(-c5ccccc5-c5cccc6c5oc5ccccc56)c5ccccc45)c4cccc1c4c23] 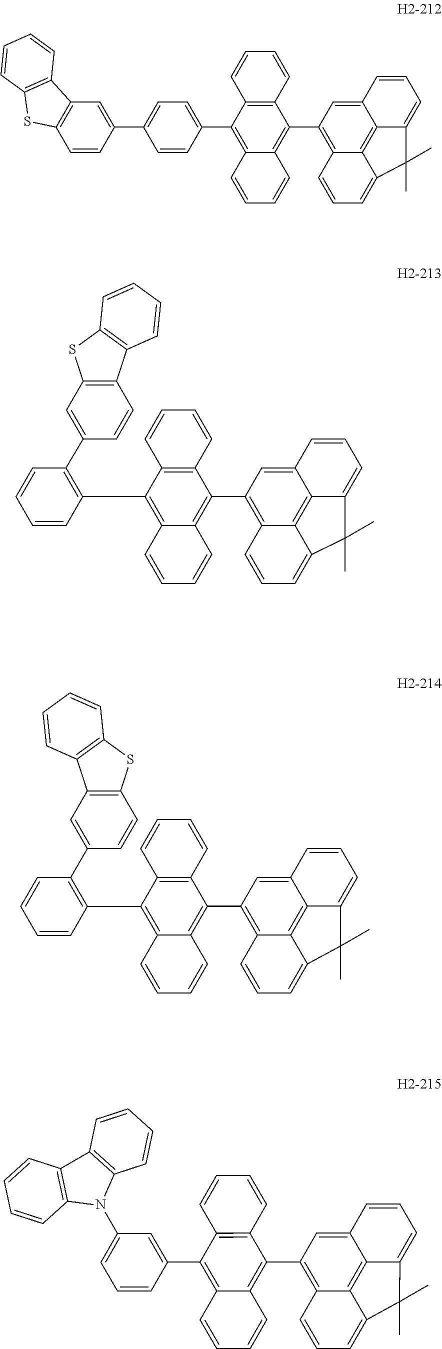 [SMILES: CC1(C)c2cccc3cc(-c4c5ccccc5c(-c5ccc(-c6ccc7sc8ccccc8c7c6)cc5)c5ccccc45)c4cccc1c4c23 ; CC1(C)c2cccc3cc(-c4c5ccccc5c(-c5ccccc5-c5ccc6c(c5)sc5ccccc56)c5ccccc45)c4cccc1c4c23 ; CC1(C)c2cccc3cc(-c4c5ccccc5c(-c5ccccc5-c5ccc6sc7ccccc7c6c5)c5ccccc45)c4cccc1c4c23 ; CC1(C)c2cccc3cc(-c4c5ccccc5c(-c5cccc(-n6c7ccccc7c7ccccc76)c5)c5ccccc45)c4cccc1c4c23] 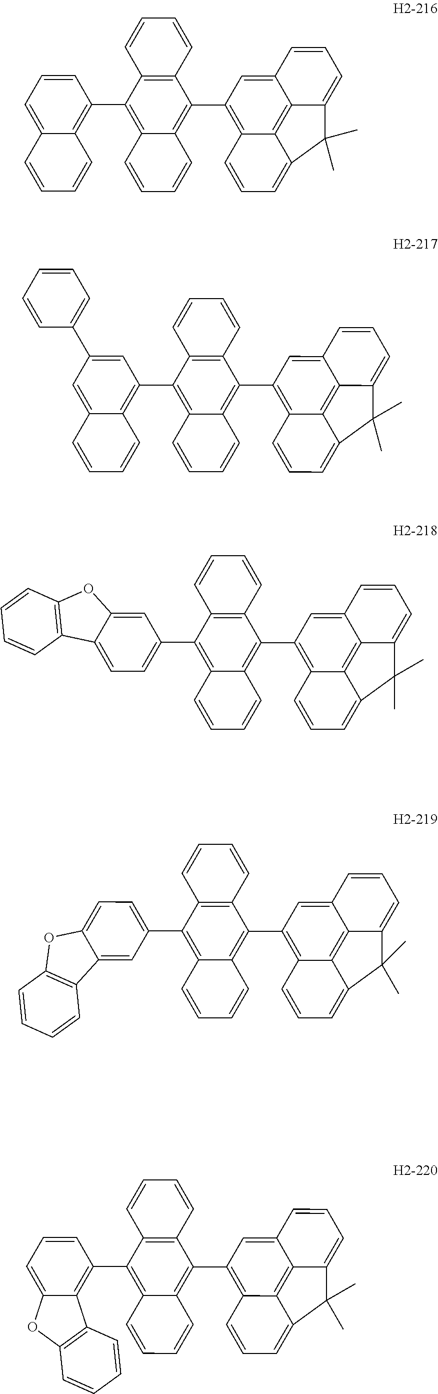 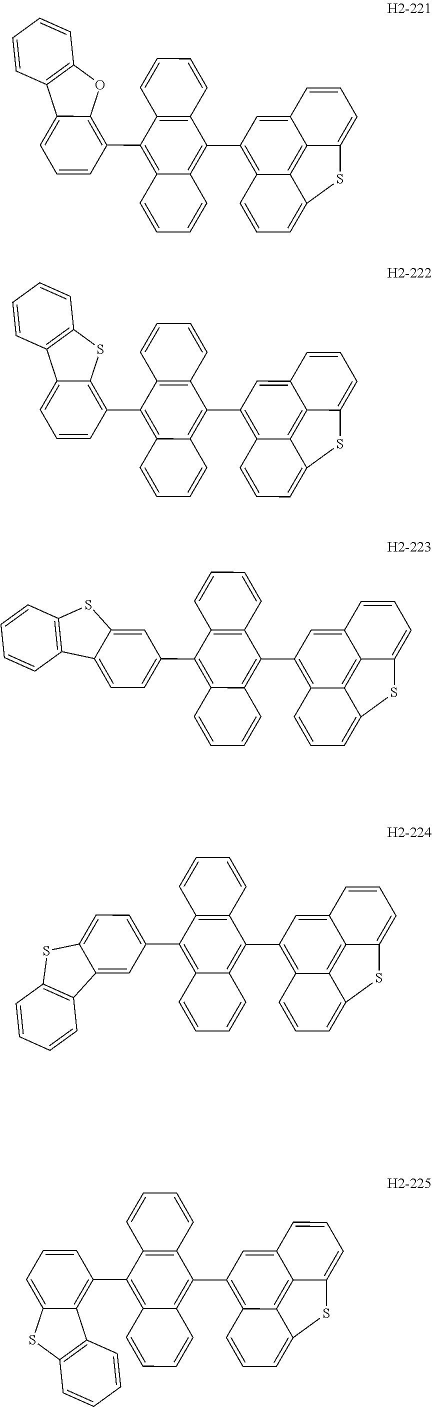 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12] 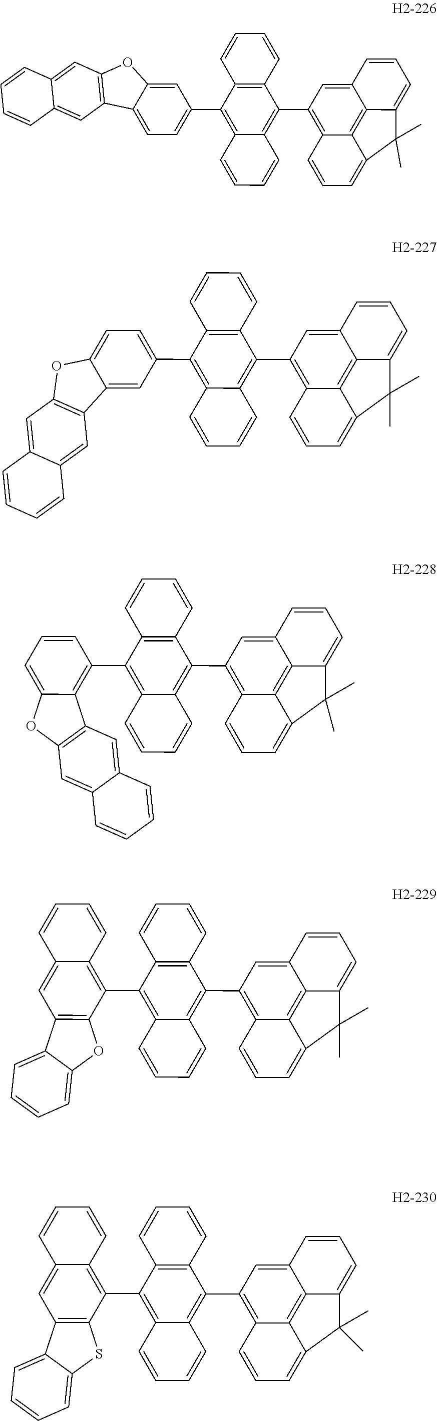 [SMILES: CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23 ; CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23] 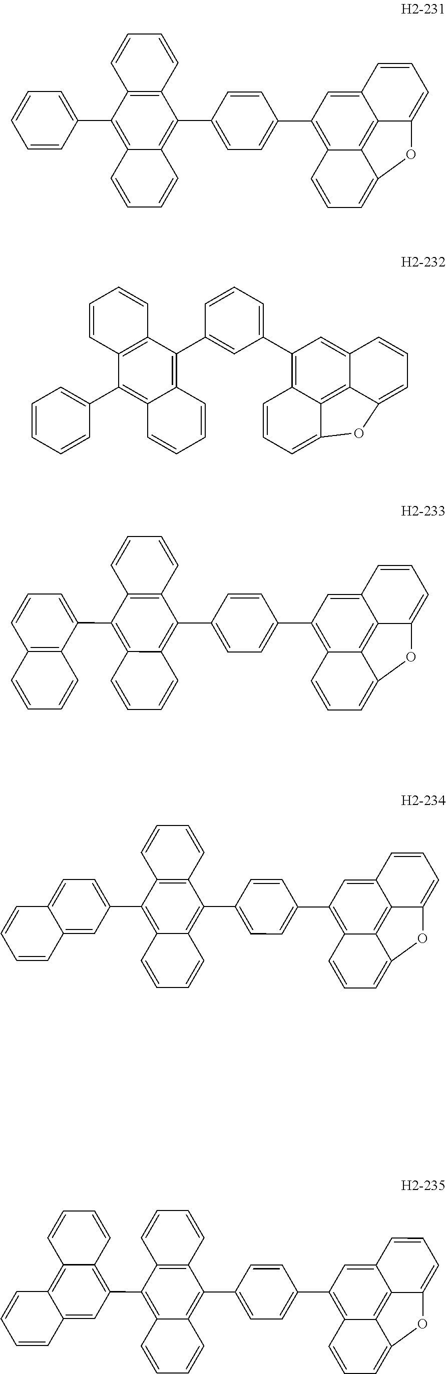 [SMILES: c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12] 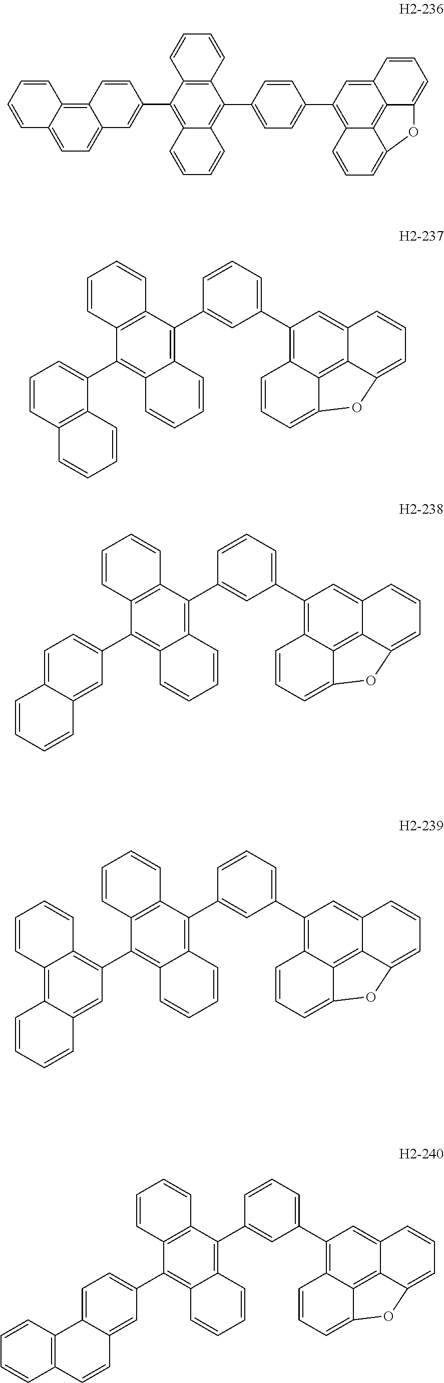 [SMILES: c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1] 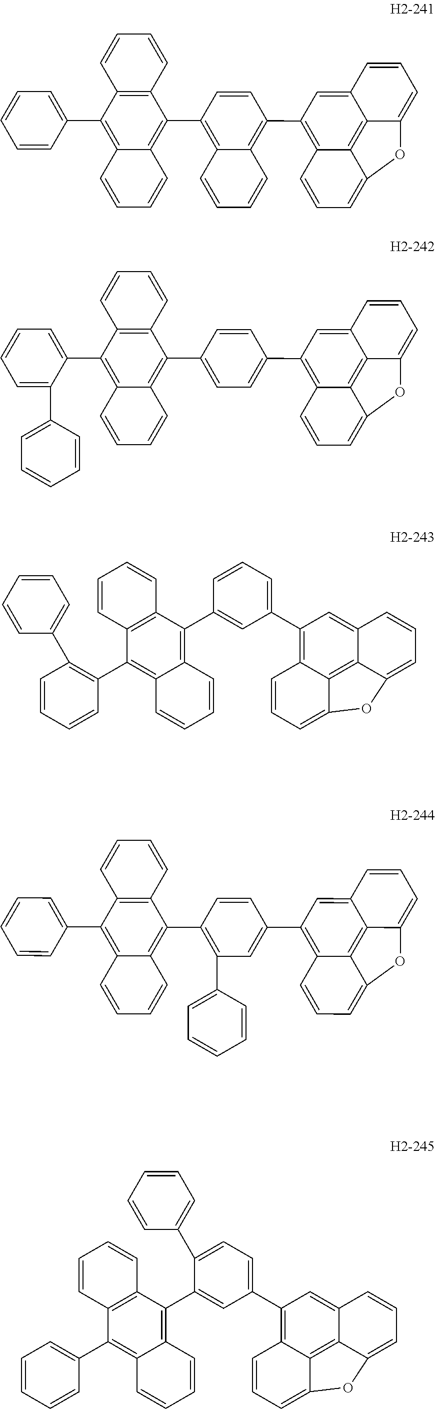 [SMILES: c1ccc(-c2c3ccccc3c(-c3ccc(-c4cc5cccc6oc7cccc4c7c56)c4ccccc34)c3ccccc23)cc1 ; c1ccc(-c2ccccc2-c2c3ccccc3c(-c3ccc(-c4cc5cccc6oc7cccc4c7c56)cc3)c3ccccc23)cc1 ; c1ccc(-c2ccccc2-c2c3ccccc3c(-c3cccc(-c4cc5cccc6oc7cccc4c7c56)c3)c3ccccc23)cc1 ; c1ccc(-c2cc(-c3cc4cccc5oc6cccc3c6c45)ccc2-c2c3ccccc3c(-c3ccccc3)c3ccccc23)cc1 ; c1ccc(-c2ccc(-c3cc4cccc5oc6cccc3c6c45)cc2-c2c3ccccc3c(-c3ccccc3)c3ccccc23)cc1] 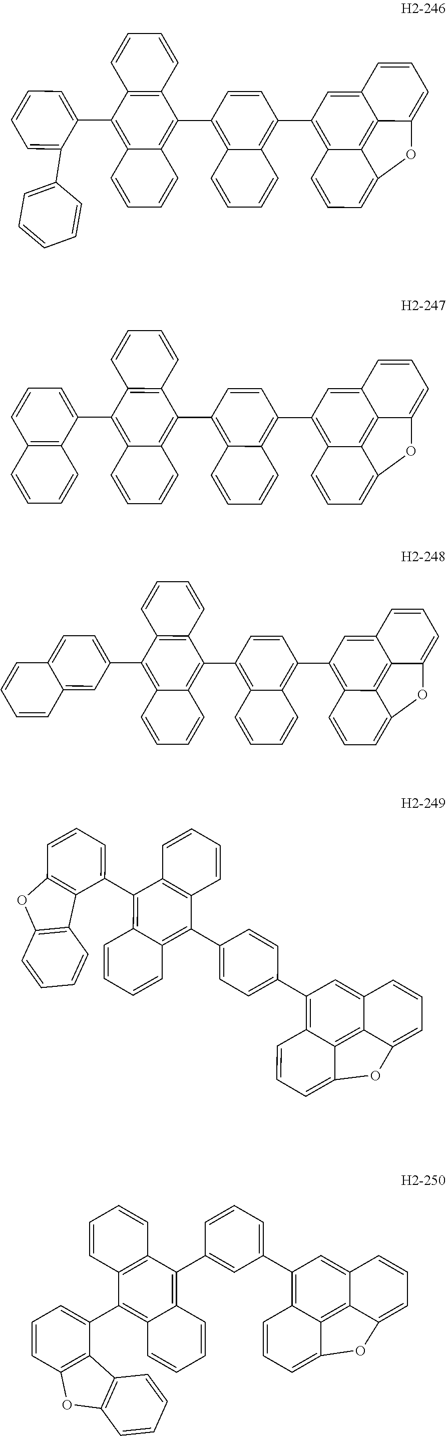 [SMILES: c1ccc(-c2ccccc2-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1] 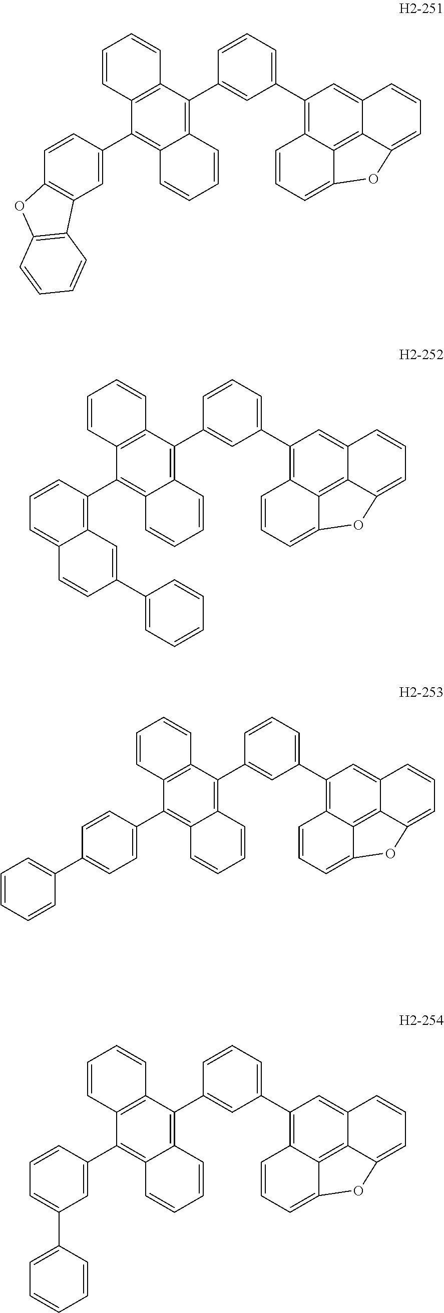 [SMILES: c1cc(-c2c3ccccc3c(-c3ccc4oc5ccccc5c4c3)c3ccccc23)cc(-c2cc3cccc4oc5cccc2c5c34)c1 ; c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1 ; c1ccc(-c2cccc(-c3c4ccccc4c(-c4cccc(-c5cc6cccc7oc8cccc5c8c67)c4)c4ccccc34)c2)cc1] 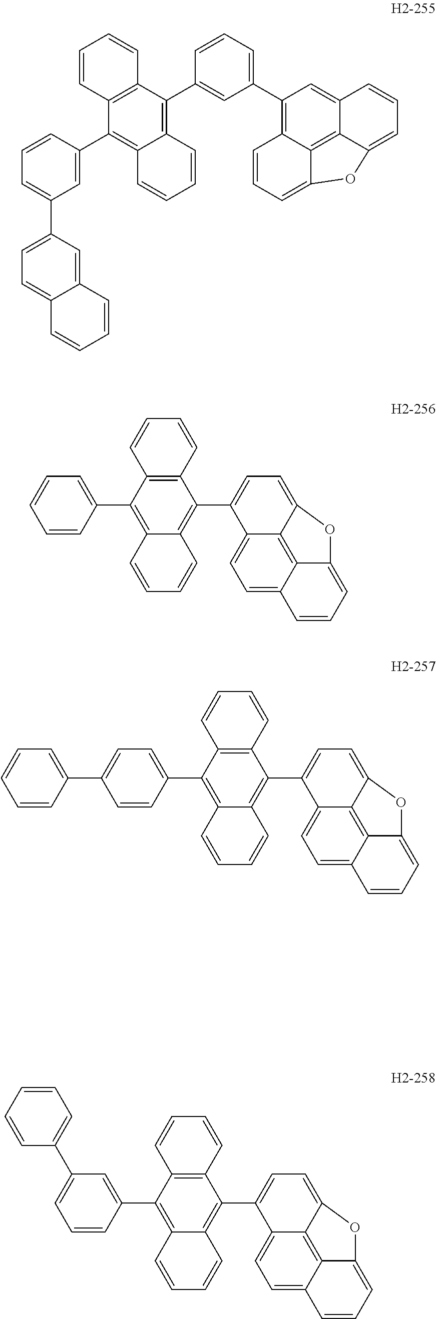 [SMILES: c1cc(-c2ccc3ccccc3c2)cc(-c2c3ccccc3c(-c3cccc(-c4cc5cccc6oc7cccc4c7c56)c3)c3ccccc23)c1 ; c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1] 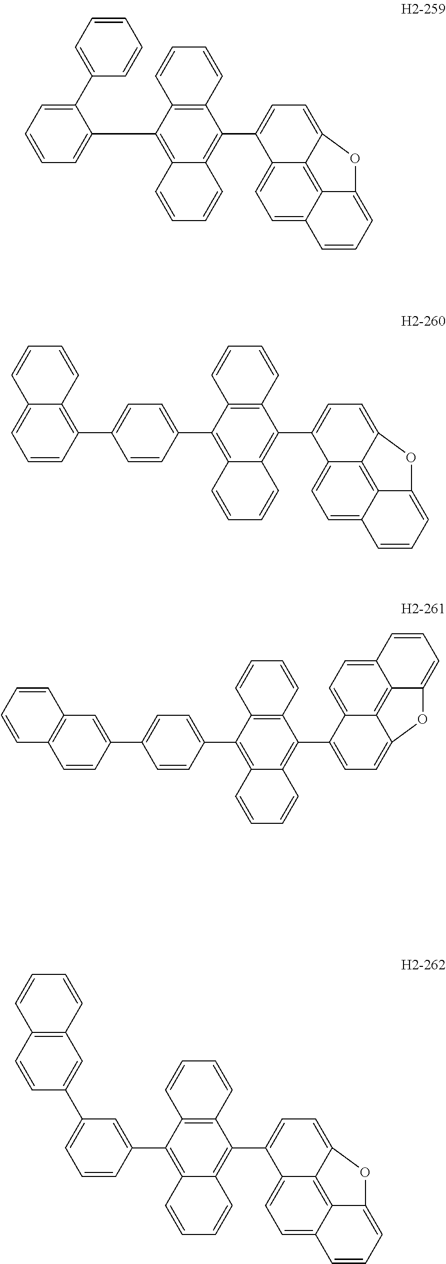 [SMILES: c1ccc(-c2ccccc2-c2c3ccccc3c(-c3ccc4oc5cccc6ccc3c4c65)c3ccccc23)cc1 ; c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1 ; c1cc(-c2ccc3ccccc3c2)cc(-c2c3ccccc3c(-c3ccc4oc5cccc6ccc3c4c65)c3ccccc23)c1] 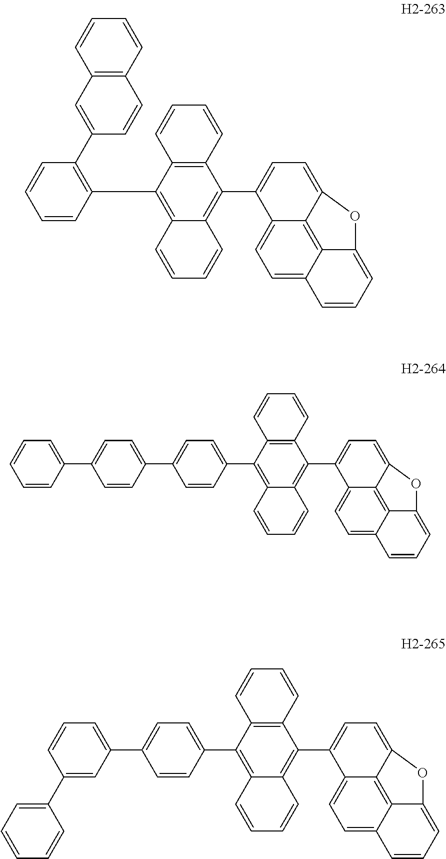 [SMILES: c1ccc(-c2c3ccccc3c(-c3ccc4oc5cccc6ccc3c4c65)c3ccccc23)c(-c2ccc3ccccc3c2)c1 ; c1ccc(-c2ccc(-c3ccc(-c4c5ccccc5c(-c5ccc6oc7cccc8ccc5c6c87)c5ccccc45)cc3)cc2)cc1 ; c1ccc(-c2cccc(-c3ccc(-c4c5ccccc5c(-c5ccc6oc7cccc8ccc5c6c87)c5ccccc45)cc3)c2)cc1] 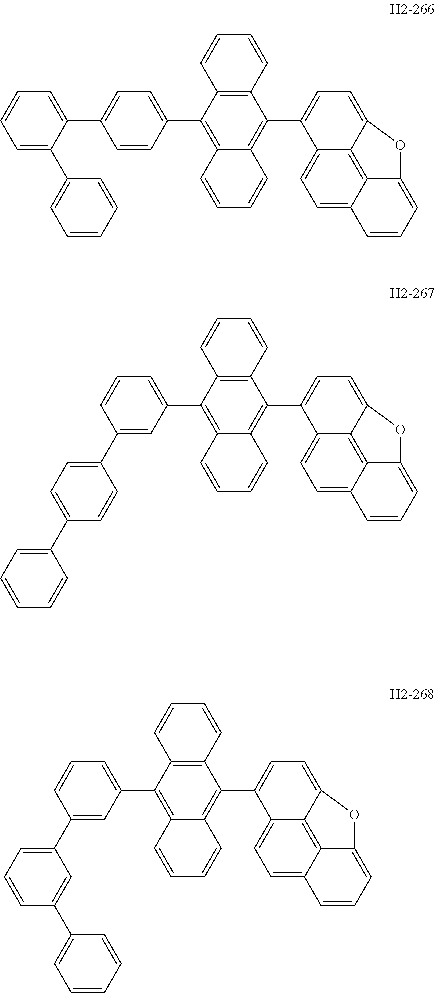 [SMILES: c1ccc(-c2ccccc2-c2ccc(-c3c4ccccc4c(-c4ccc5oc6cccc7ccc4c5c76)c4ccccc34)cc2)cc1 ; c1ccc(-c2ccc(-c3cccc(-c4c5ccccc5c(-c5ccc6oc7cccc8ccc5c6c87)c5ccccc45)c3)cc2)cc1 ; c1ccc(-c2cccc(-c3cccc(-c4c5ccccc5c(-c5ccc6oc7cccc8ccc5c6c87)c5ccccc45)c3)c2)cc1] 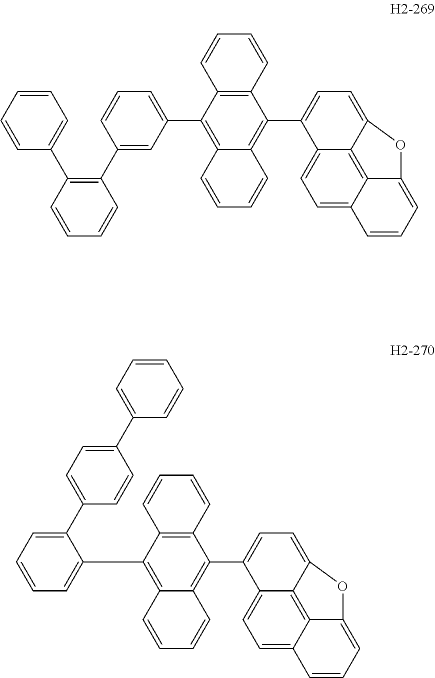 [SMILES: c1ccc(-c2ccccc2-c2cccc(-c3c4ccccc4c(-c4ccc5oc6cccc7ccc4c5c76)c4ccccc34)c2)cc1 ; c1ccc(-c2ccc(-c3ccccc3-c3c4ccccc4c(-c4ccc5oc6cccc7ccc4c5c76)c4ccccc34)cc2)cc1] 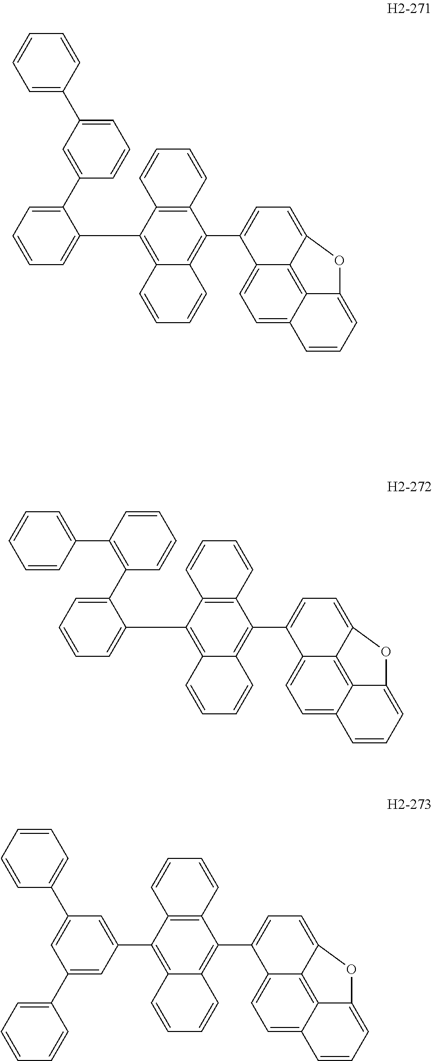 [SMILES: c1ccc(-c2cccc(-c3ccccc3-c3c4ccccc4c(-c4ccc5oc6cccc7ccc4c5c76)c4ccccc34)c2)cc1 ; c1ccc(-c2ccccc2-c2ccccc2-c2c3ccccc3c(-c3ccc4oc5cccc6ccc3c4c65)c3ccccc23)cc1 ; c1ccc(-c2cc(-c3ccccc3)cc(-c3c4ccccc4c(-c4ccc5oc6cccc7ccc4c5c76)c4ccccc34)c2)cc1] 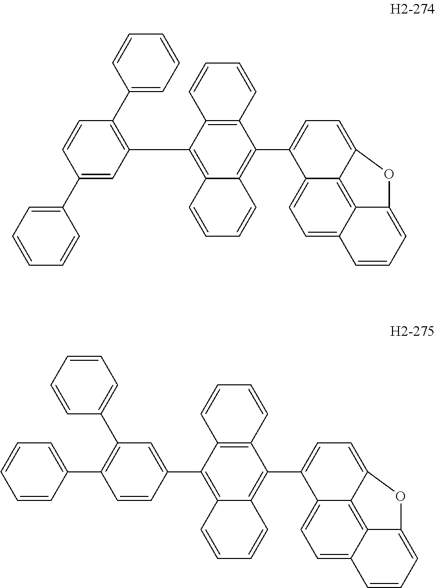 [SMILES: c1ccc(-c2ccc(-c3ccccc3)c(-c3c4ccccc4c(-c4ccc5oc6cccc7ccc4c5c76)c4ccccc34)c2)cc1 ; c1ccc(-c2ccc(-c3c4ccccc4c(-c4ccc5oc6cccc7ccc4c5c76)c4ccccc34)cc2-c2ccccc2)cc1] 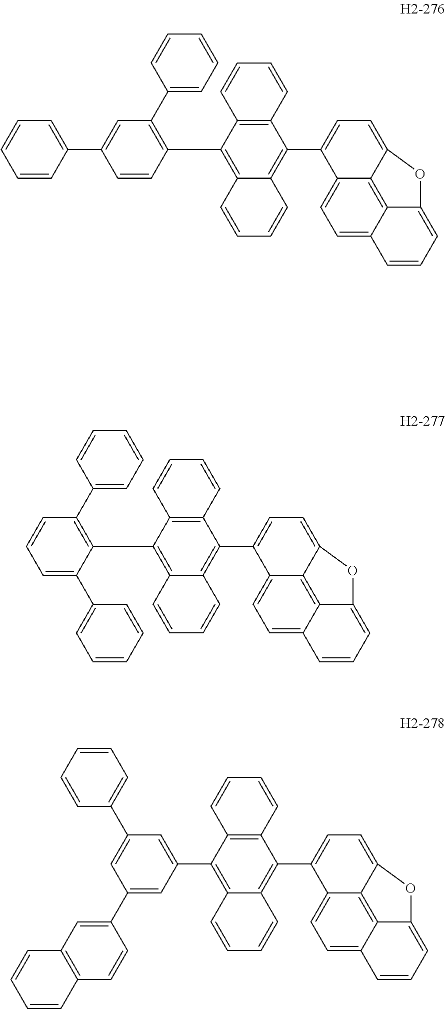 [SMILES: c1ccc(-c2ccc(-c3c4ccccc4c(-c4ccc5oc6cccc7ccc4c5c76)c4ccccc34)c(-c3ccccc3)c2)cc1 ; c1ccc(-c2cccc(-c3ccccc3)c2-c2c3ccccc3c(-c3ccc4oc5cccc6ccc3c4c65)c3ccccc23)cc1 ; c1ccc(-c2cc(-c3ccc4ccccc4c3)cc(-c3c4ccccc4c(-c4ccc5oc6cccc7ccc4c5c76)c4ccccc34)c2)cc1] 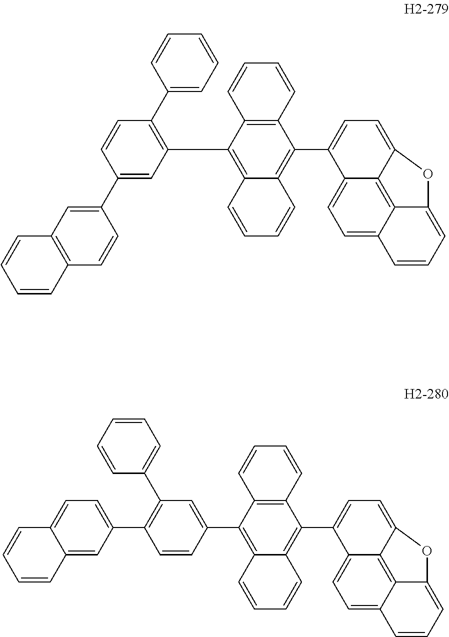 [SMILES: c1ccc(-c2ccc(-c3ccc4ccccc4c3)cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)ccc2-c2ccc3ccccc3c2)cc1] 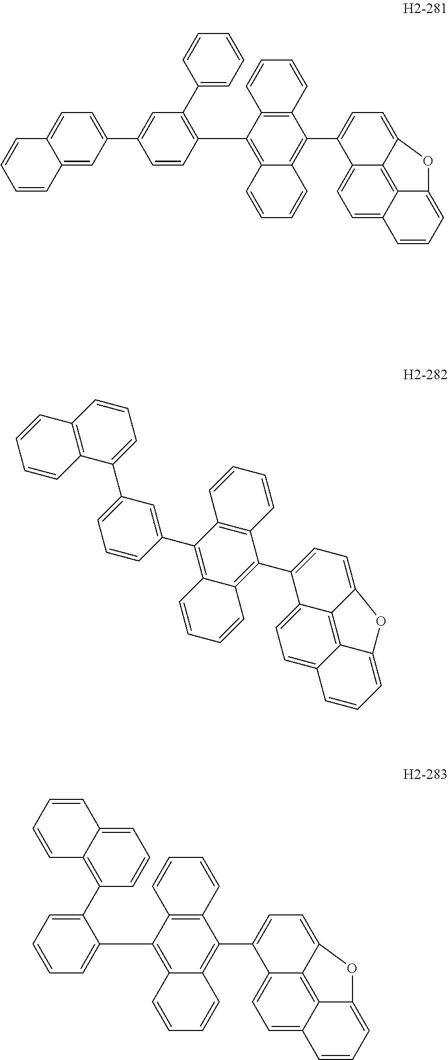 [SMILES: c1ccc(-c2cc(-c3ccc4ccccc4c3)ccc2-c2c3ccccc3c(-c3ccc4oc5cccc6ccc3c4c65)c3ccccc23)cc1 ; c1cc(-c2cccc3ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2cccc3ccccc23)c1] 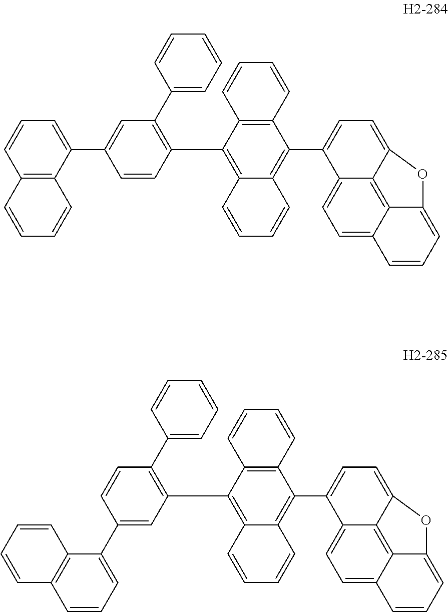 [SMILES: c1ccc(-c2cc(-c3cccc4ccccc34)ccc2-c2c3ccccc3c(-c3ccc4oc5cccc6ccc3c4c65)c3ccccc23)cc1 ; c1ccc(-c2ccc(-c3cccc4ccccc34)cc2-c2c3ccccc3c(-c3ccc4oc5cccc6ccc3c4c65)c3ccccc23)cc1] 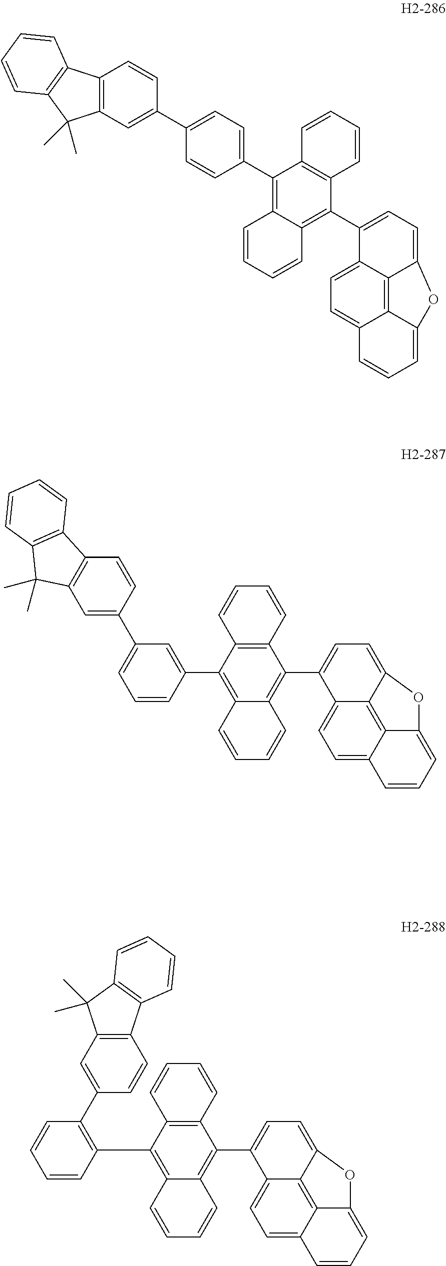 [SMILES: CC1(C)c2ccccc2-c2ccc(-c3ccc(-c4c5ccccc5c(-c5ccc6oc7cccc8ccc5c6c87)c5ccccc45)cc3)cc21 ; CC1(C)c2ccccc2-c2ccc(-c3cccc(-c4c5ccccc5c(-c5ccc6oc7cccc8ccc5c6c87)c5ccccc45)c3)cc21 ; CC1(C)c2ccccc2-c2ccc(-c3ccccc3-c3c4ccccc4c(-c4ccc5oc6cccc7ccc4c5c76)c4ccccc34)cc21] 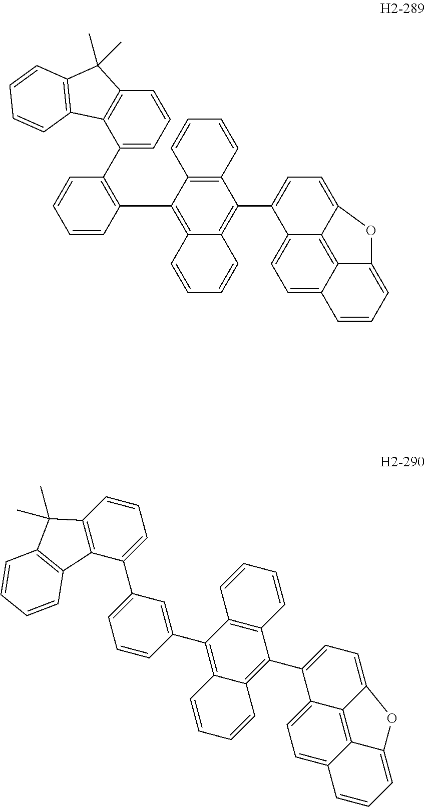 [SMILES: CC1(C)c2ccccc2-c2c(-c3ccccc3-c3c4ccccc4c(-c4ccc5oc6cccc7ccc4c5c76)c4ccccc34)cccc21 ; CC1(C)c2ccccc2-c2c(-c3cccc(-c4c5ccccc5c(-c5ccc6oc7cccc8ccc5c6c87)c5ccccc45)c3)cccc21] 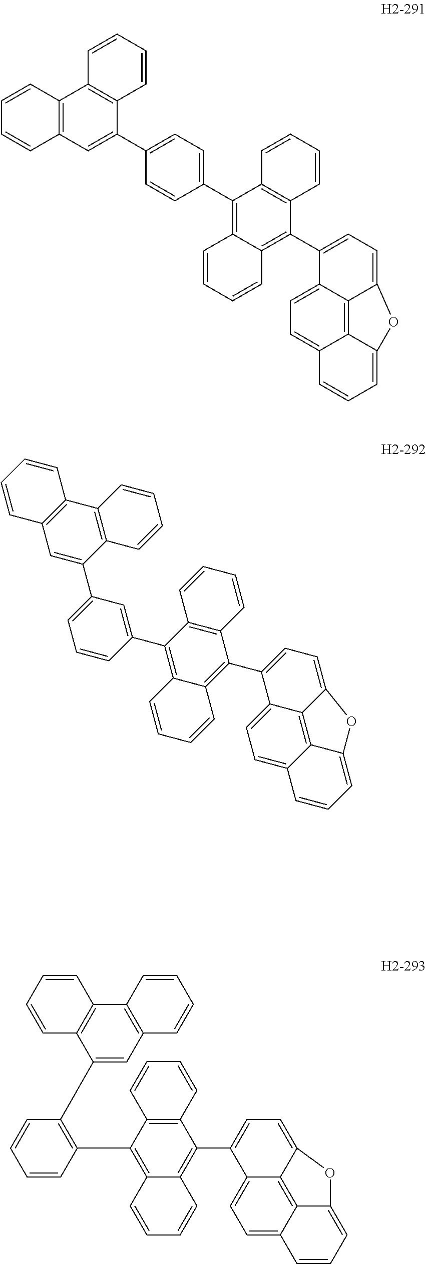 [SMILES: c1ccc2c(c1)cc(-c1ccc(-c3c4ccccc4c(-c4ccc5oc6cccc7ccc4c5c76)c4ccccc34)cc1)c1ccccc12 ; c1cc(-c2c3ccccc3c(-c3ccc4oc5cccc6ccc3c4c65)c3ccccc23)cc(-c2cc3ccccc3c3ccccc23)c1 ; c1ccc(-c2cc3ccccc3c3ccccc23)c(-c2c3ccccc3c(-c3ccc4oc5cccc6ccc3c4c65)c3ccccc23)c1] 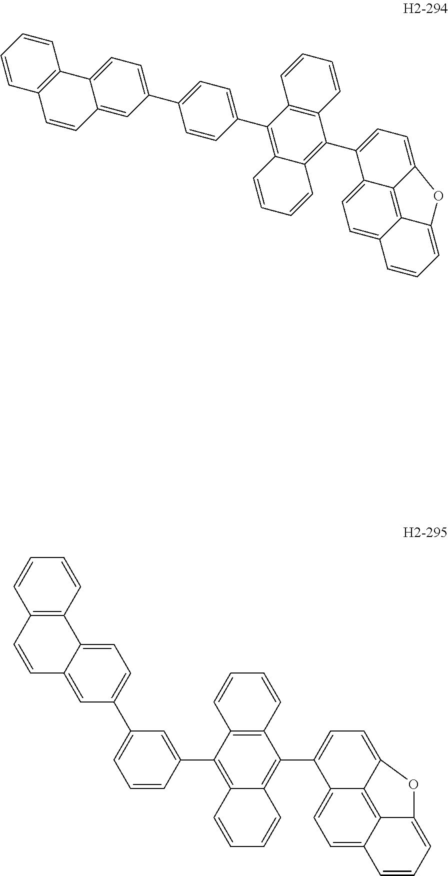 [SMILES: c1ccc2c(c1)ccc1cc(-c3ccc(-c4c5ccccc5c(-c5ccc6oc7cccc8ccc5c6c87)c5ccccc45)cc3)ccc12 ; c1cc(-c2ccc3c(ccc4ccccc43)c2)cc(-c2c3ccccc3c(-c3ccc4oc5cccc6ccc3c4c65)c3ccccc23)c1] 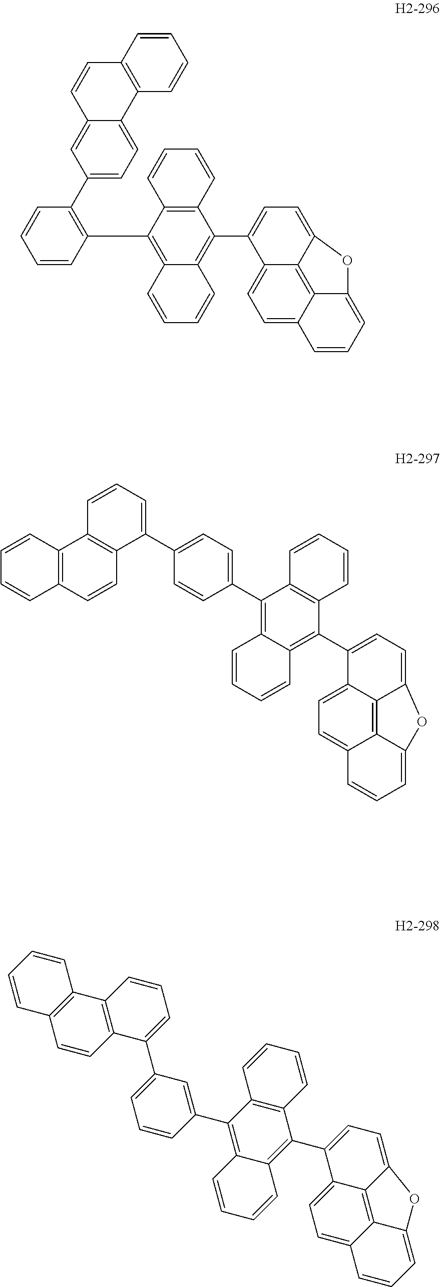 [SMILES: c1ccc(-c2c3ccccc3c(-c3ccc4oc5cccc6ccc3c4c65)c3ccccc23)c(-c2ccc3c(ccc4ccccc43)c2)c1 ; c1ccc2c(c1)ccc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2ccc2ccccc23)c1] 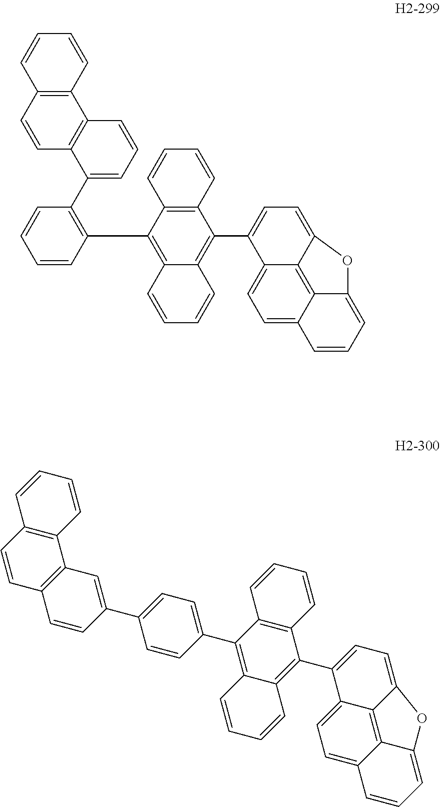 [SMILES: c1ccc(-c2cccc3c2ccc2ccccc23)c(-c2c3ccccc3c(-c3ccc4oc5cccc6ccc3c4c65)c3ccccc23)c1 ; c1ccc2c(c1)ccc1ccc(-c3ccc(-c4c5ccccc5c(-c5ccc6oc7cccc8ccc5c6c87)c5ccccc45)cc3)cc12] 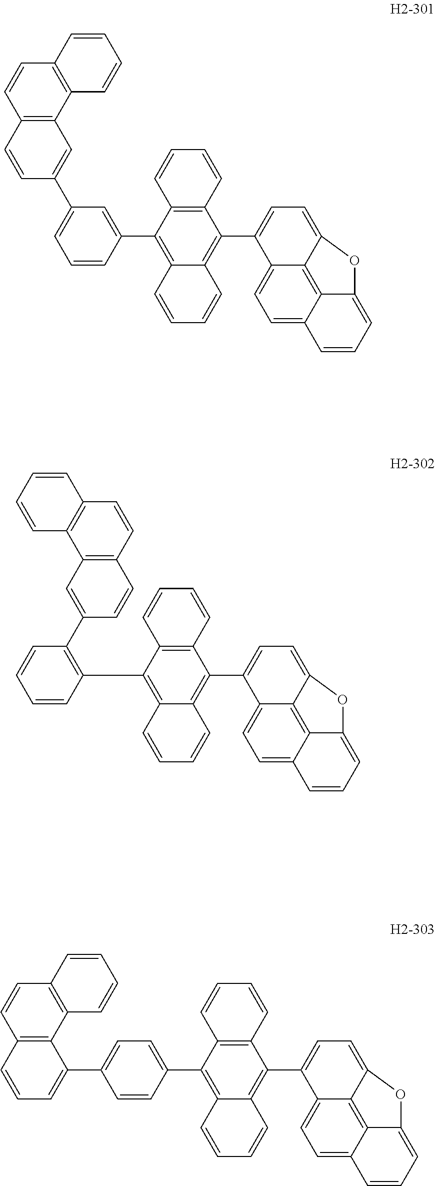 [SMILES: c1cc(-c2ccc3ccc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccc4ccccc4c3c2)c1 ; c1ccc2c(c1)ccc1cccc(-c3ccc(-c4c5ccccc5c(-c5ccc6oc7cccc8ccc5c6c87)c5ccccc45)cc3)c12] 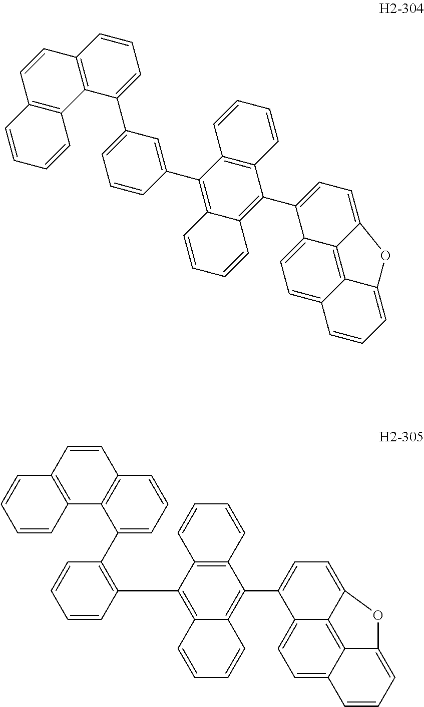 [SMILES: c1cc(-c2c3ccccc3c(-c3ccc4oc5cccc6ccc3c4c65)c3ccccc23)cc(-c2cccc3ccc4ccccc4c23)c1 ; c1ccc(-c2cccc3ccc4ccccc4c23)c(-c2c3ccccc3c(-c3ccc4oc5cccc6ccc3c4c65)c3ccccc23)c1] 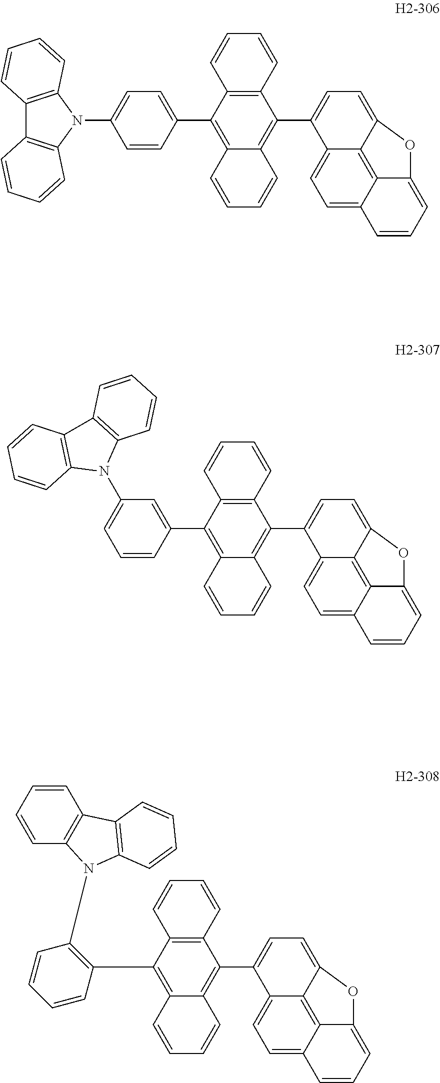 [SMILES: c1cc2ccc3c(-c4c5ccccc5c(-c5ccc(-n6c7ccccc7c7ccccc76)cc5)c5ccccc45)ccc4oc(c1)c2c43 ; c1cc(-c2c3ccccc3c(-c3ccc4oc5cccc6ccc3c4c65)c3ccccc23)cc(-n2c3ccccc3c3ccccc32)c1 ; c1ccc(-n2c3ccccc3c3ccccc32)c(-c2c3ccccc3c(-c3ccc4oc5cccc6ccc3c4c65)c3ccccc23)c1] 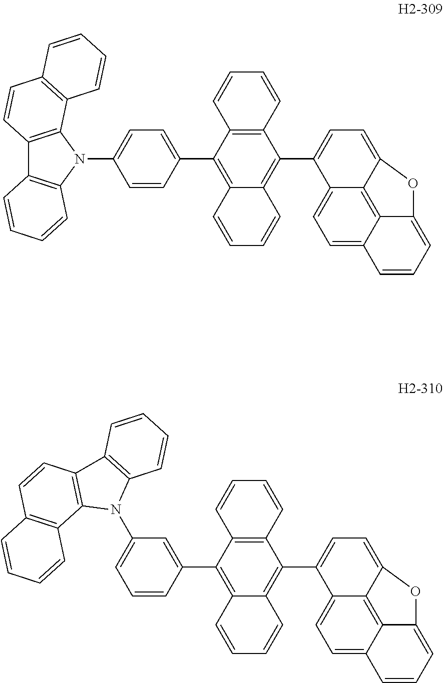 [SMILES: c1ccc2c(c1)ccc1c3ccccc3n(-c3ccc(-c4c5ccccc5c(-c5ccc6oc7cccc8ccc5c6c87)c5ccccc45)cc3)c21 ; c1cc(-c2c3ccccc3c(-c3ccc4oc5cccc6ccc3c4c65)c3ccccc23)cc(-n2c3ccccc3c3ccc4ccccc4c32)c1] 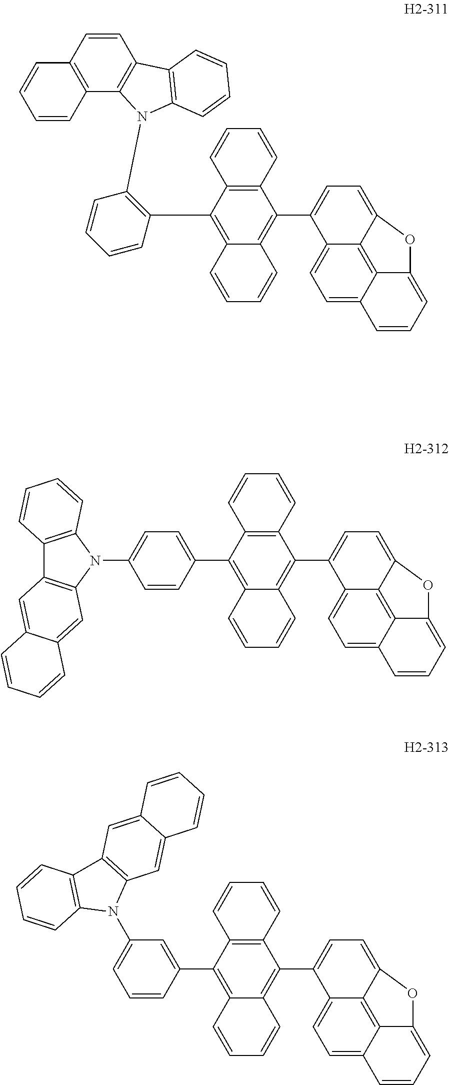 [SMILES: c1ccc(-n2c3ccccc3c3ccc4ccccc4c32)c(-c2c3ccccc3c(-c3ccc4oc5cccc6ccc3c4c65)c3ccccc23)c1 ; c1ccc2cc3c(cc2c1)c1ccccc1n3-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3cc4ccccc4cc32)c1] 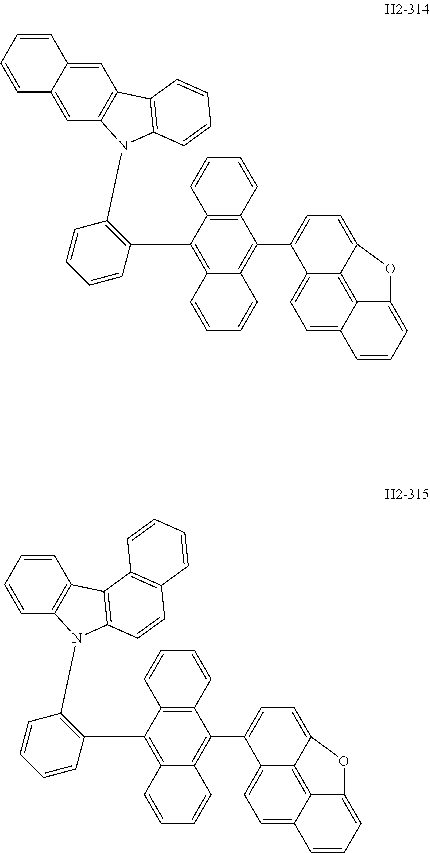 [SMILES: c1ccc(-n2c3ccccc3c3cc4ccccc4cc32)c(-c2c3ccccc3c(-c3ccc4oc5cccc6ccc3c4c65)c3ccccc23)c1 ; c1ccc(-n2c3ccccc3c3c4ccccc4ccc32)c(-c2c3ccccc3c(-c3ccc4oc5cccc6ccc3c4c65)c3ccccc23)c1] 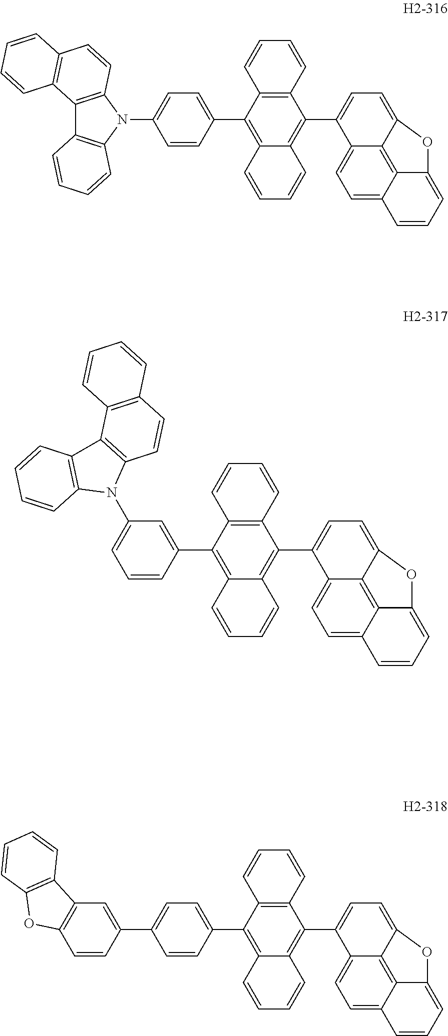 [SMILES: c1ccc2c(c1)ccc1c2c2ccccc2n1-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3c4ccccc4ccc32)c1 ; c1ccc2c(c1)oc1ccc(-c3ccc(-c4c5ccccc5c(-c5ccc6oc7cccc8ccc5c6c87)c5ccccc45)cc3)cc12] 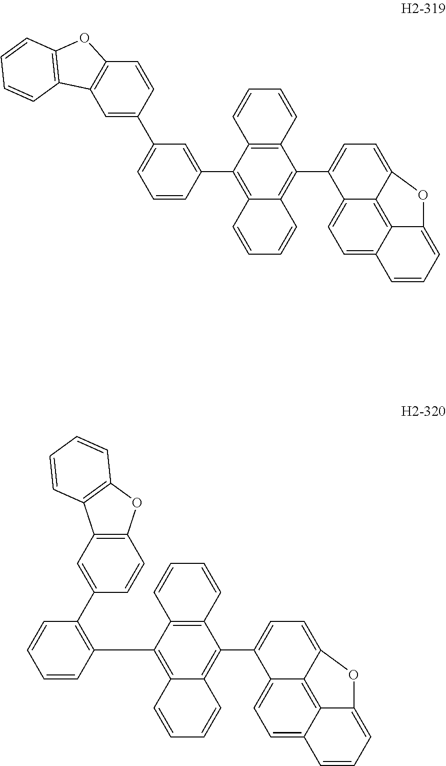 [SMILES: c1cc(-c2ccc3oc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3oc4ccccc4c3c2)c1] 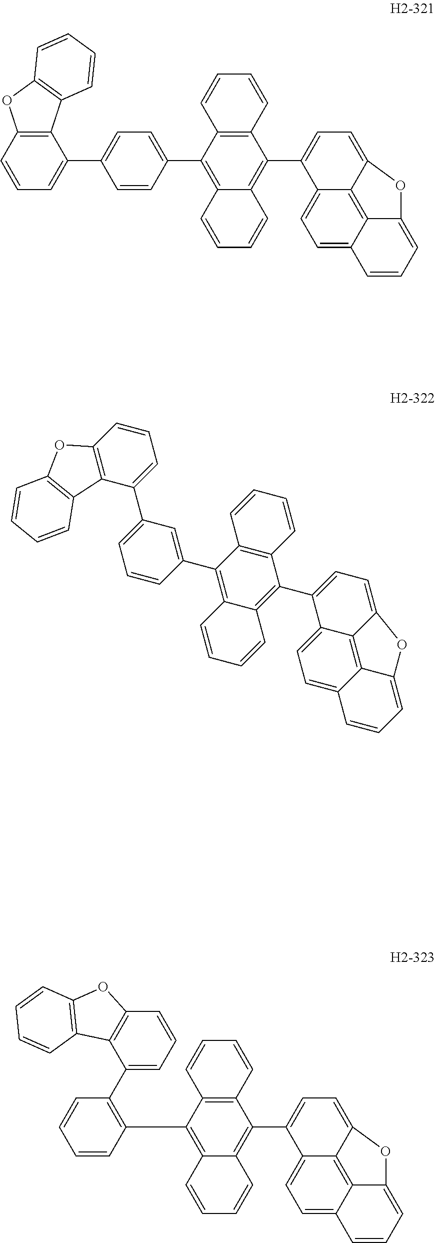 [SMILES: c1ccc2c(c1)oc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3oc4ccccc4c23)c1 ; c1ccc(-c2cccc3oc4ccccc4c23)c(-c2c3ccccc3c(-c3ccc4oc5cccc6ccc3c4c65)c3ccccc23)c1] 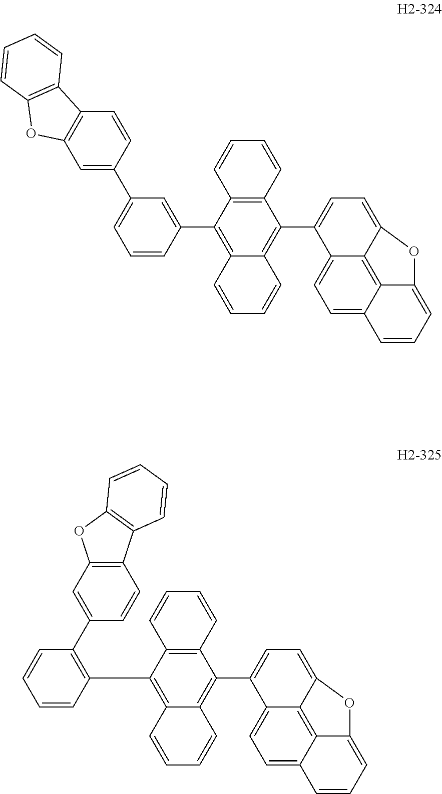 [SMILES: c1cc(-c2ccc3c(c2)oc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)oc2ccccc23)c1] 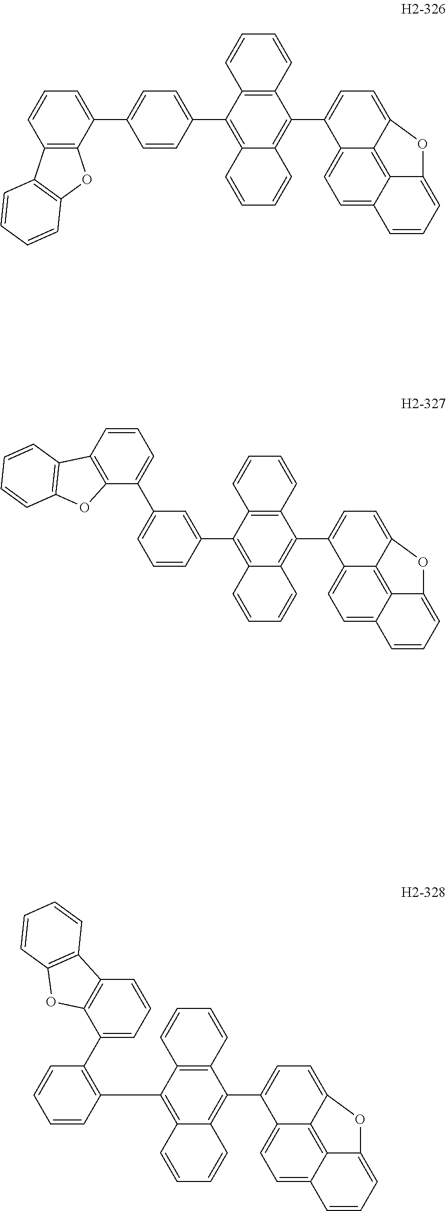 [SMILES: c1ccc2c(c1)oc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2oc2ccccc23)c1 ; c1ccc(-c2cccc3c2oc2ccccc23)c(-c2c3ccccc3c(-c3ccc4oc5cccc6ccc3c4c65)c3ccccc23)c1] 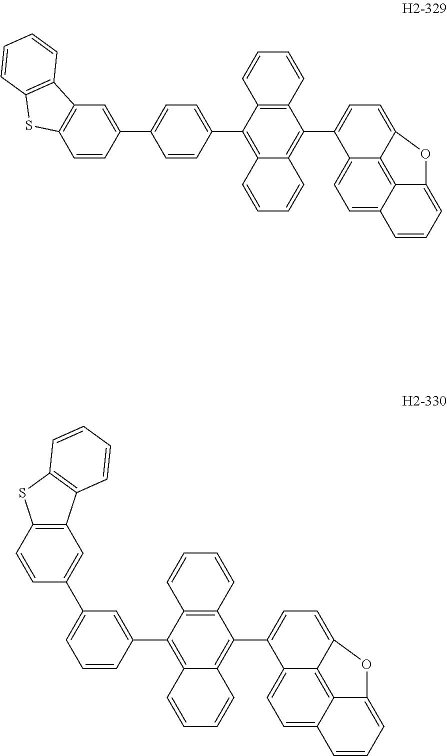 [SMILES: c1cc2ccc3c(-c4c5ccccc5c(-c5ccc(-c6ccc7sc8ccccc8c7c6)cc5)c5ccccc45)ccc4oc(c1)c2c43 ; c1cc(-c2ccc3sc4ccccc4c3c2)cc(-c2c3ccccc3c(-c3ccc4oc5cccc6ccc3c4c65)c3ccccc23)c1] 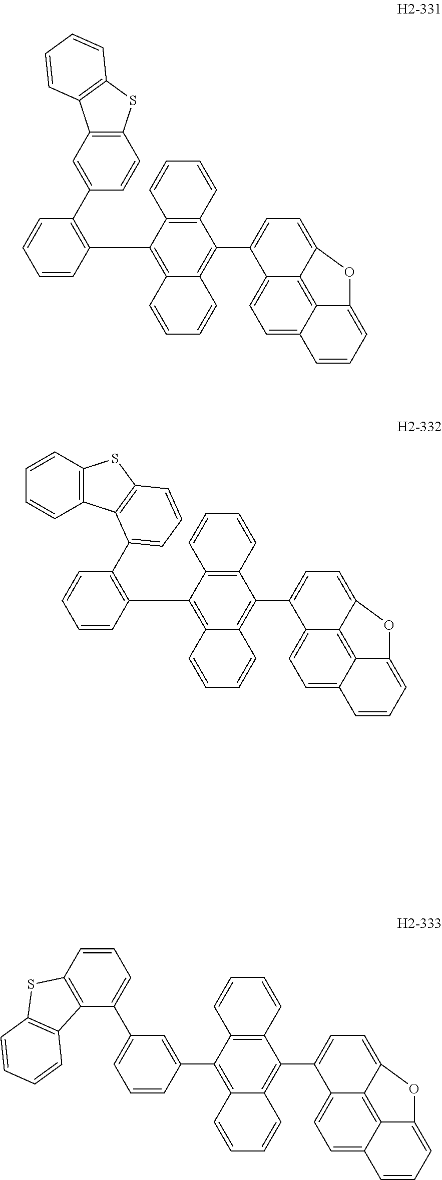 [SMILES: c1ccc(-c2c3ccccc3c(-c3ccc4oc5cccc6ccc3c4c65)c3ccccc23)c(-c2ccc3sc4ccccc4c3c2)c1 ; c1ccc(-c2cccc3sc4ccccc4c23)c(-c2c3ccccc3c(-c3ccc4oc5cccc6ccc3c4c65)c3ccccc23)c1 ; c1cc(-c2c3ccccc3c(-c3ccc4oc5cccc6ccc3c4c65)c3ccccc23)cc(-c2cccc3sc4ccccc4c23)c1] 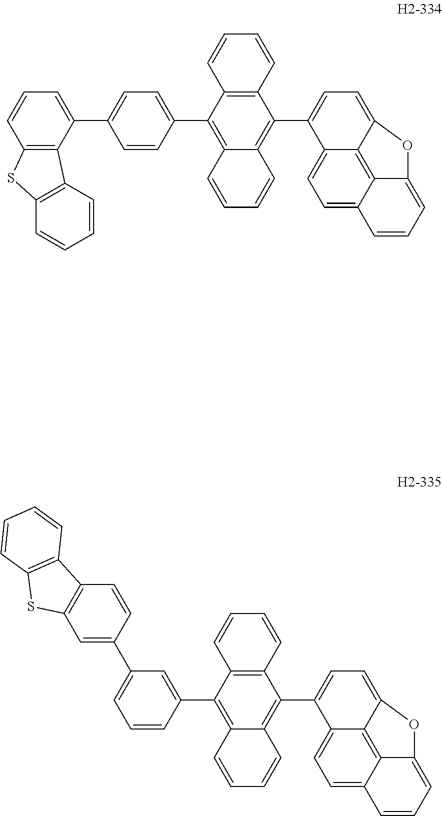 [SMILES: c1cc2ccc3c(-c4c5ccccc5c(-c5ccc(-c6cccc7sc8ccccc8c67)cc5)c5ccccc45)ccc4oc(c1)c2c43 ; c1cc(-c2ccc3c(c2)sc2ccccc23)cc(-c2c3ccccc3c(-c3ccc4oc5cccc6ccc3c4c65)c3ccccc23)c1] 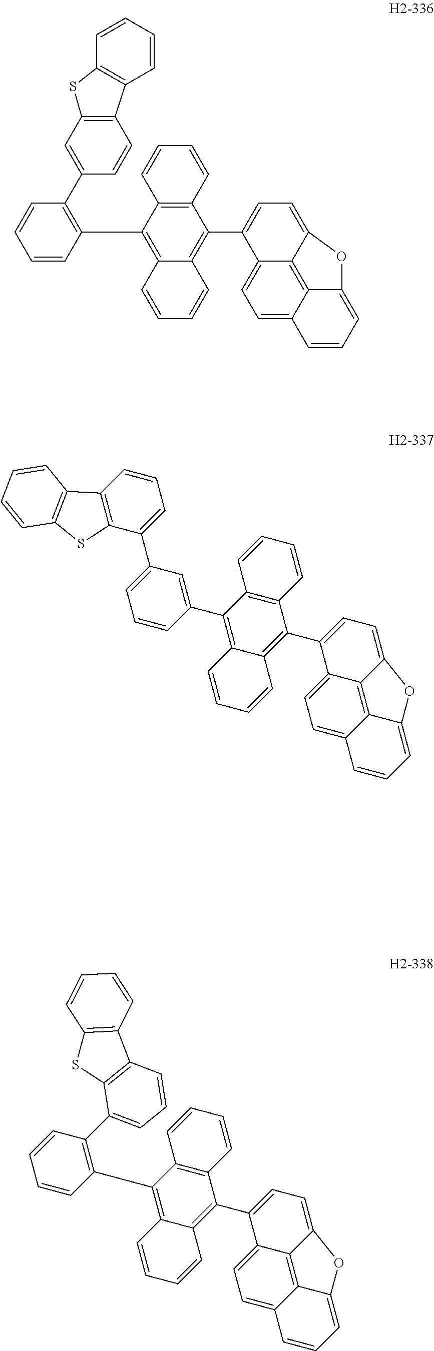 [SMILES: c1ccc(-c2c3ccccc3c(-c3ccc4oc5cccc6ccc3c4c65)c3ccccc23)c(-c2ccc3c(c2)sc2ccccc23)c1 ; c1cc(-c2c3ccccc3c(-c3ccc4oc5cccc6ccc3c4c65)c3ccccc23)cc(-c2cccc3c2sc2ccccc23)c1 ; c1ccc(-c2cccc3c2sc2ccccc23)c(-c2c3ccccc3c(-c3ccc4oc5cccc6ccc3c4c65)c3ccccc23)c1] 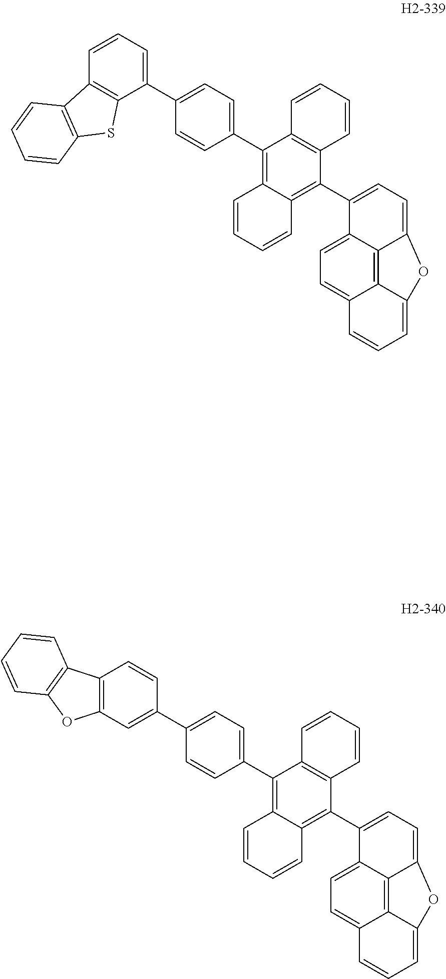 [SMILES: c1cc2ccc3c(-c4c5ccccc5c(-c5ccc(-c6cccc7c6sc6ccccc67)cc5)c5ccccc45)ccc4oc(c1)c2c43 ; c1ccc2c(c1)oc1cc(-c3ccc(-c4c5ccccc5c(-c5ccc6oc7cccc8ccc5c6c87)c5ccccc45)cc3)ccc12] 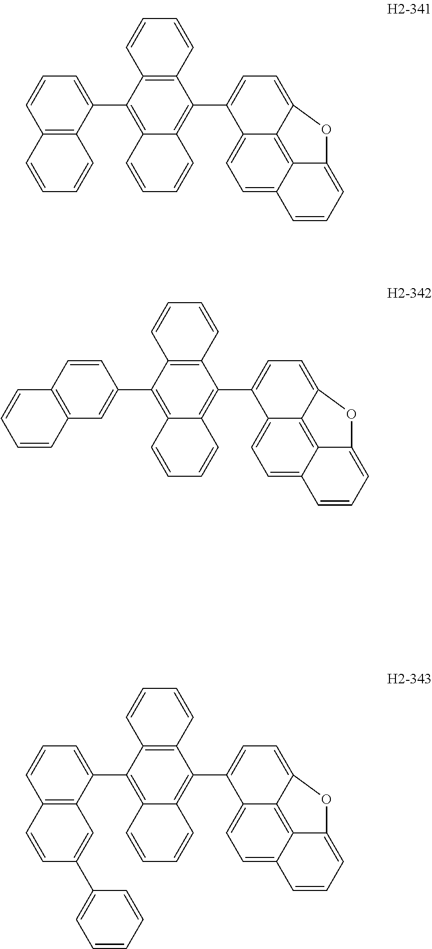 [SMILES: c1ccc2c(-c3c4ccccc4c(-c4ccc5oc6cccc7ccc4c5c76)c4ccccc34)cccc2c1 ; c1ccc2cc(-c3c4ccccc4c(-c4ccc5oc6cccc7ccc4c5c76)c4ccccc34)ccc2c1 ; c1ccc(-c2ccc3cccc(-c4c5ccccc5c(-c5ccc6oc7cccc8ccc5c6c87)c5ccccc45)c3c2)cc1] 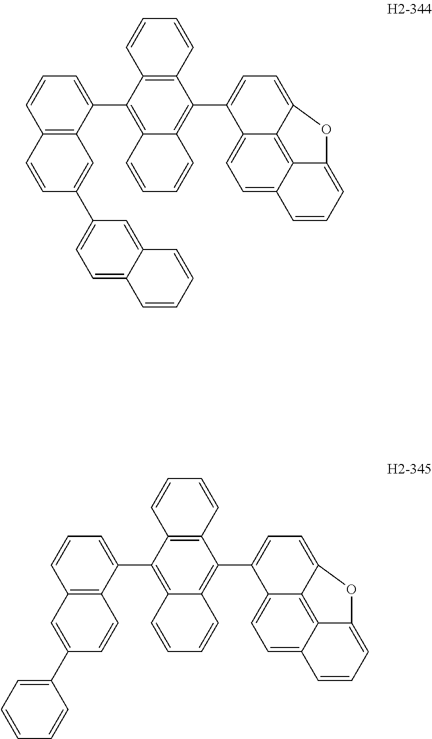 [SMILES: c1ccc2cc(-c3ccc4cccc(-c5c6ccccc6c(-c6ccc7oc8cccc9ccc6c7c98)c6ccccc56)c4c3)ccc2c1 ; c1ccc(-c2ccc3c(-c4c5ccccc5c(-c5ccc6oc7cccc8ccc5c6c87)c5ccccc45)cccc3c2)cc1] 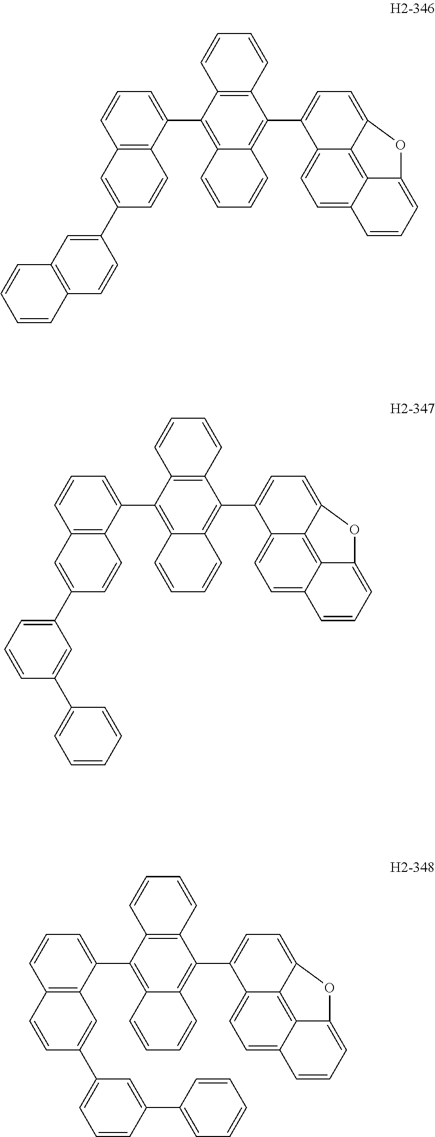 [SMILES: c1ccc2cc(-c3ccc4c(-c5c6ccccc6c(-c6ccc7oc8cccc9ccc6c7c98)c6ccccc56)cccc4c3)ccc2c1 ; c1ccc(-c2cccc(-c3ccc4c(-c5c6ccccc6c(-c6ccc7oc8cccc9ccc6c7c98)c6ccccc56)cccc4c3)c2)cc1 ; c1ccc(-c2cccc(-c3ccc4cccc(-c5c6ccccc6c(-c6ccc7oc8cccc9ccc6c7c98)c6ccccc56)c4c3)c2)cc1] 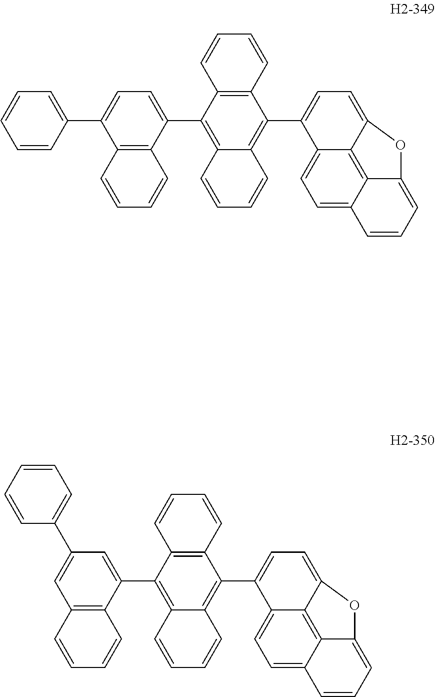 [SMILES: c1ccc(-c2ccc(-c3c4ccccc4c(-c4ccc5oc6cccc7ccc4c5c76)c4ccccc34)c3ccccc23)cc1 ; c1ccc(-c2cc(-c3c4ccccc4c(-c4ccc5oc6cccc7ccc4c5c76)c4ccccc34)c3ccccc3c2)cc1] 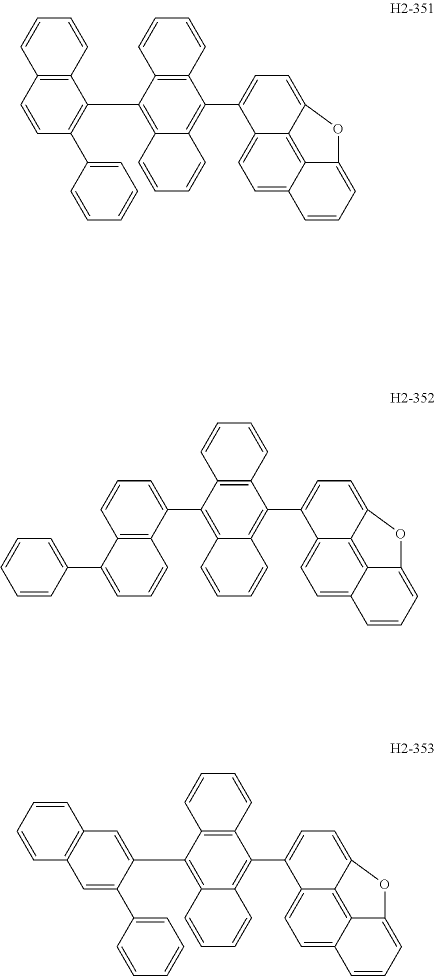 [SMILES: c1ccc(-c2ccc3ccccc3c2-c2c3ccccc3c(-c3ccc4oc5cccc6ccc3c4c65)c3ccccc23)cc1 ; c1ccc(-c2cccc3c(-c4c5ccccc5c(-c5ccc6oc7cccc8ccc5c6c87)c5ccccc45)cccc23)cc1 ; c1ccc(-c2cc3ccccc3cc2-c2c3ccccc3c(-c3ccc4oc5cccc6ccc3c4c65)c3ccccc23)cc1] 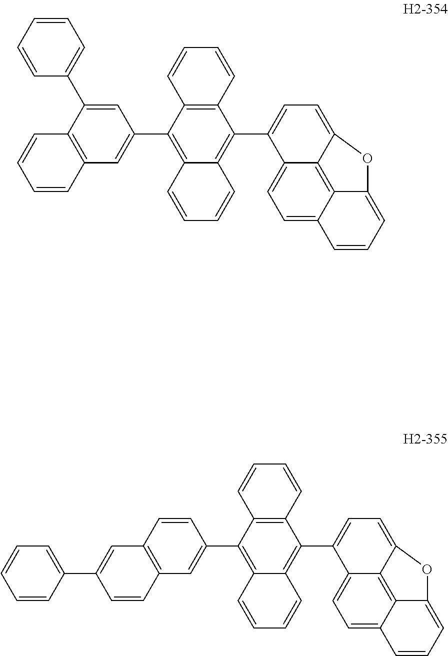 [SMILES: c1ccc(-c2cc(-c3c4ccccc4c(-c4ccc5oc6cccc7ccc4c5c76)c4ccccc34)cc3ccccc23)cc1 ; c1ccc(-c2ccc3cc(-c4c5ccccc5c(-c5ccc6oc7cccc8ccc5c6c87)c5ccccc45)ccc3c2)cc1] 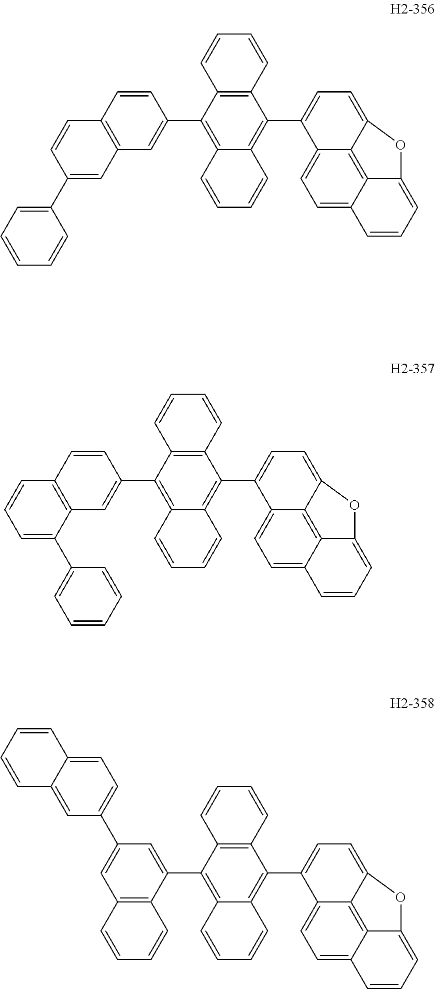 [SMILES: c1ccc(-c2ccc3ccc(-c4c5ccccc5c(-c5ccc6oc7cccc8ccc5c6c87)c5ccccc45)cc3c2)cc1 ; c1ccc(-c2cccc3ccc(-c4c5ccccc5c(-c5ccc6oc7cccc8ccc5c6c87)c5ccccc45)cc23)cc1 ; c1ccc2cc(-c3cc(-c4c5ccccc5c(-c5ccc6oc7cccc8ccc5c6c87)c5ccccc45)c4ccccc4c3)ccc2c1] 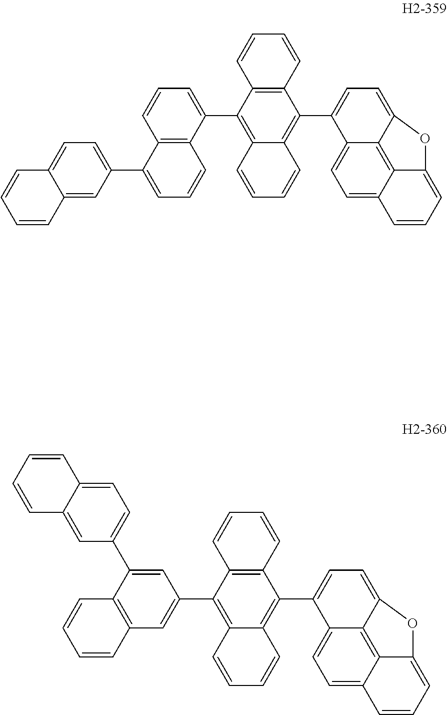 [SMILES: c1ccc2cc(-c3cccc4c(-c5c6ccccc6c(-c6ccc7oc8cccc9ccc6c7c98)c6ccccc56)cccc34)ccc2c1 ; c1ccc2cc(-c3cc(-c4c5ccccc5c(-c5ccc6oc7cccc8ccc5c6c87)c5ccccc45)cc4ccccc34)ccc2c1] 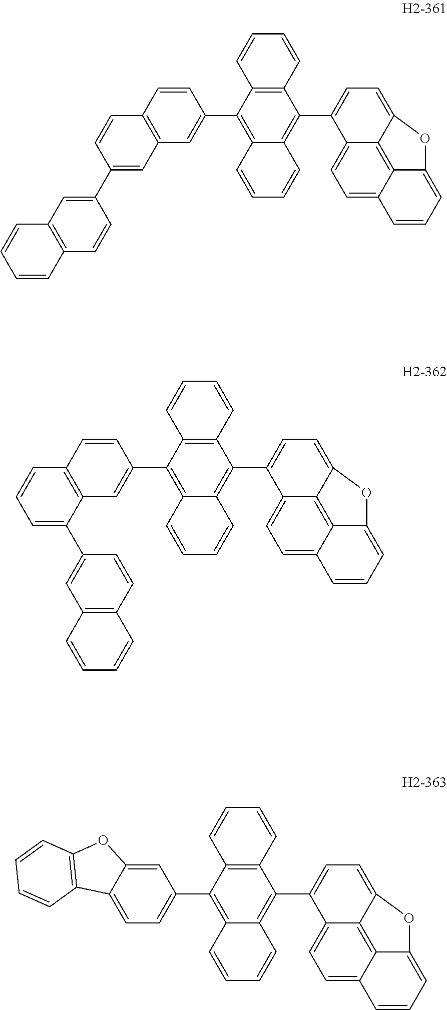 [SMILES: c1ccc2cc(-c3ccc4ccc(-c5c6ccccc6c(-c6ccc7oc8cccc9ccc6c7c98)c6ccccc56)cc4c3)ccc2c1 ; c1ccc2cc(-c3cccc4ccc(-c5c6ccccc6c(-c6ccc7oc8cccc9ccc6c7c98)c6ccccc56)cc34)ccc2c1 ; c1ccc2c(c1)oc1cc(-c3c4ccccc4c(-c4ccc5oc6cccc7ccc4c5c76)c4ccccc34)ccc12] 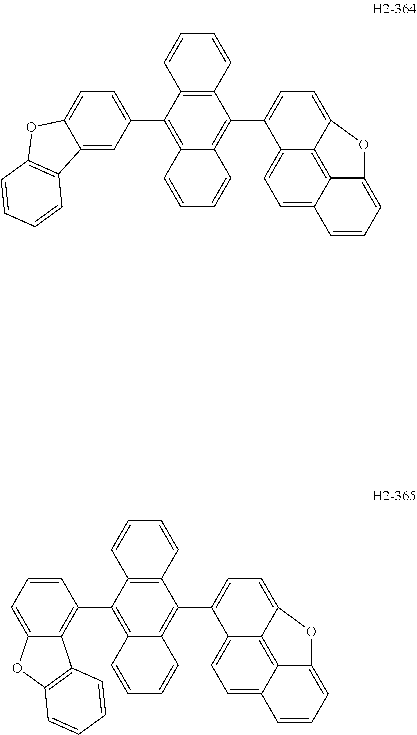 [SMILES: c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12] 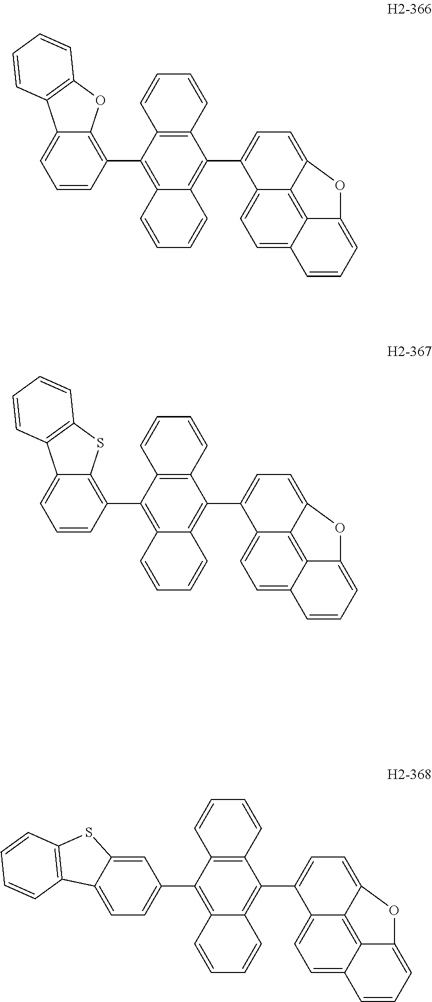 [SMILES: c1ccc2c(c1)oc1c(-c3c4ccccc4c(-c4ccc5oc6cccc7ccc4c5c76)c4ccccc34)cccc12 ; c1cc2ccc3c(-c4c5ccccc5c(-c5cccc6c5sc5ccccc56)c5ccccc45)ccc4oc(c1)c2c43 ; c1cc2ccc3c(-c4c5ccccc5c(-c5ccc6c(c5)sc5ccccc56)c5ccccc45)ccc4oc(c1)c2c43] 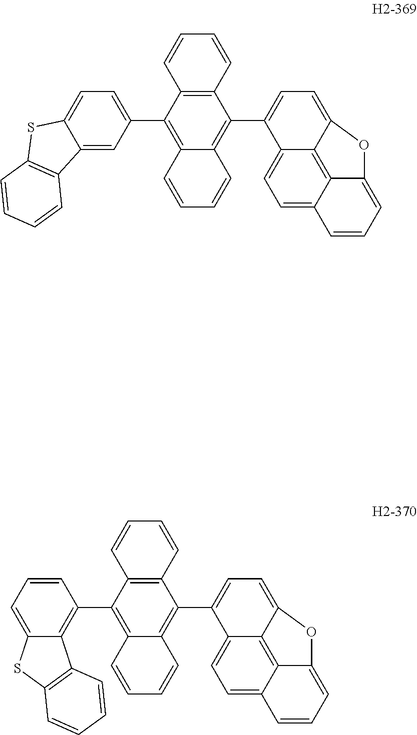 [SMILES: c1cc2ccc3c(-c4c5ccccc5c(-c5ccc6sc7ccccc7c6c5)c5ccccc45)ccc4oc(c1)c2c43 ; c1cc2ccc3c(-c4c5ccccc5c(-c5cccc6sc7ccccc7c56)c5ccccc45)ccc4oc(c1)c2c43] 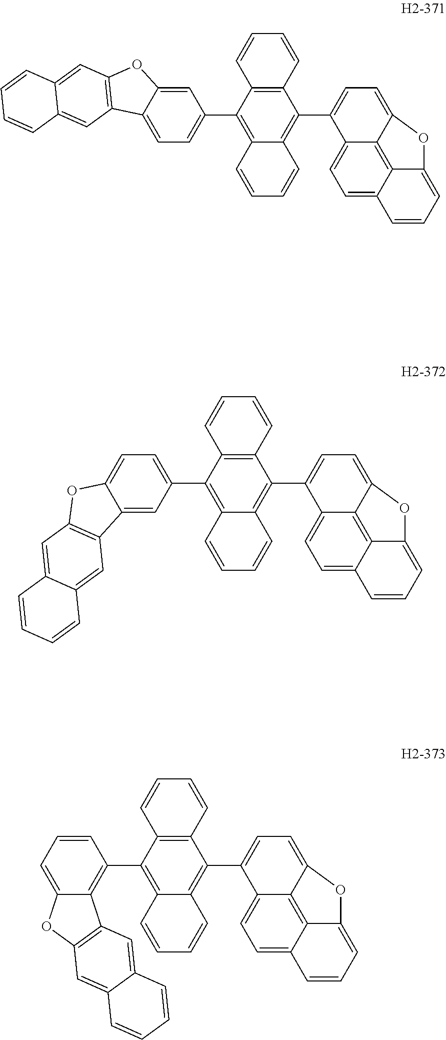 [SMILES: c1ccc2cc3c(cc2c1)oc1cc(-c2c4ccccc4c(-c4ccc5oc6cccc7ccc4c5c76)c4ccccc24)ccc13 ; c1ccc2cc3c(cc2c1)oc1ccc(-c2c4ccccc4c(-c4ccc5oc6cccc7ccc4c5c76)c4ccccc24)cc13 ; c1ccc2cc3c(cc2c1)oc1cccc(-c2c4ccccc4c(-c4ccc5oc6cccc7ccc4c5c76)c4ccccc24)c13] 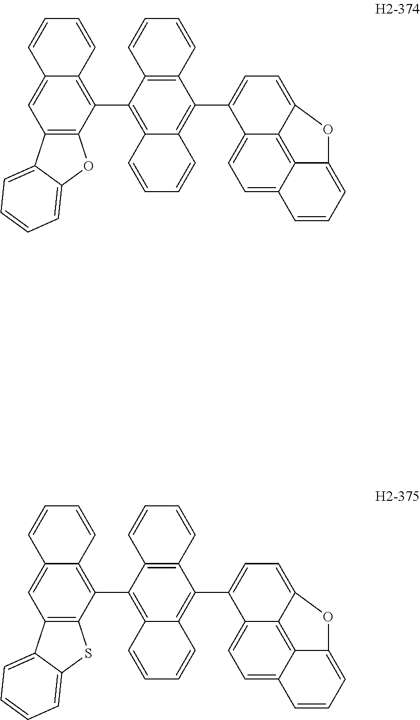 [SMILES: c1ccc2c(-c3c4ccccc4c(-c4ccc5oc6cccc7ccc4c5c76)c4ccccc34)c3oc4ccccc4c3cc2c1 ; c1ccc2c(-c3c4ccccc4c(-c4ccc5oc6cccc7ccc4c5c76)c4ccccc34)c3sc4ccccc4c3cc2c1] 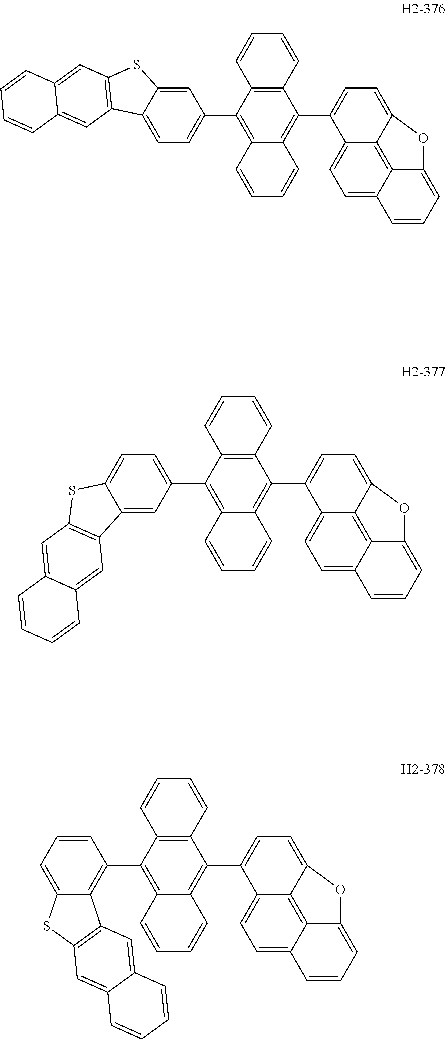 [SMILES: c1ccc2cc3c(cc2c1)sc1cc(-c2c4ccccc4c(-c4ccc5oc6cccc7ccc4c5c76)c4ccccc24)ccc13 ; c1ccc2cc3c(cc2c1)sc1ccc(-c2c4ccccc4c(-c4ccc5oc6cccc7ccc4c5c76)c4ccccc24)cc13 ; c1ccc2cc3c(cc2c1)sc1cccc(-c2c4ccccc4c(-c4ccc5oc6cccc7ccc4c5c76)c4ccccc24)c13] 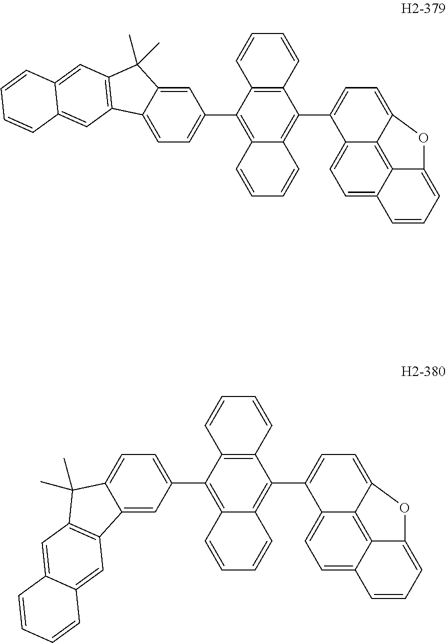 [SMILES: CC1(C)c2cc(-c3c4ccccc4c(-c4ccc5oc6cccc7ccc4c5c76)c4ccccc34)ccc2-c2cc3ccccc3cc21 ; CC1(C)c2ccc(-c3c4ccccc4c(-c4ccc5oc6cccc7ccc4c5c76)c4ccccc34)cc2-c2cc3ccccc3cc21] 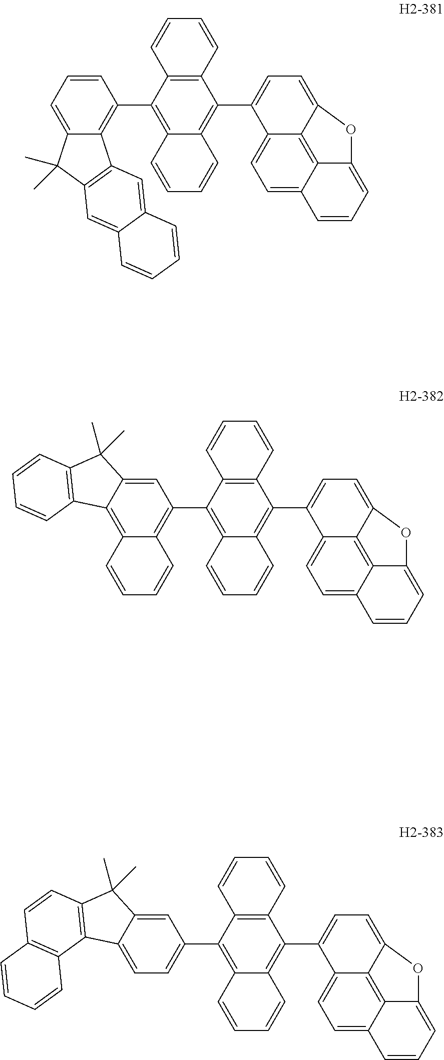 [SMILES: CC1(C)c2cc3ccccc3cc2-c2c(-c3c4ccccc4c(-c4ccc5oc6cccc7ccc4c5c76)c4ccccc34)cccc21 ; CC1(C)c2ccccc2-c2c1cc(-c1c3ccccc3c(-c3ccc4oc5cccc6ccc3c4c65)c3ccccc13)c1ccccc21 ; CC1(C)c2cc(-c3c4ccccc4c(-c4ccc5oc6cccc7ccc4c5c76)c4ccccc34)ccc2-c2c1ccc1ccccc21] 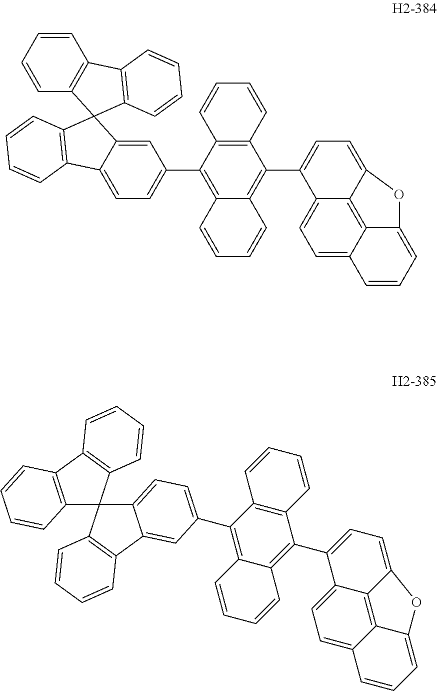 [SMILES: c1ccc2c(c1)-c1ccccc1C21c2ccccc2-c2ccc(-c3c4ccccc4c(-c4ccc5oc6cccc7ccc4c5c76)c4ccccc34)cc21 ; c1ccc2c(c1)-c1ccccc1C21c2ccccc2-c2cc(-c3c4ccccc4c(-c4ccc5oc6cccc7ccc4c5c76)c4ccccc34)ccc21] 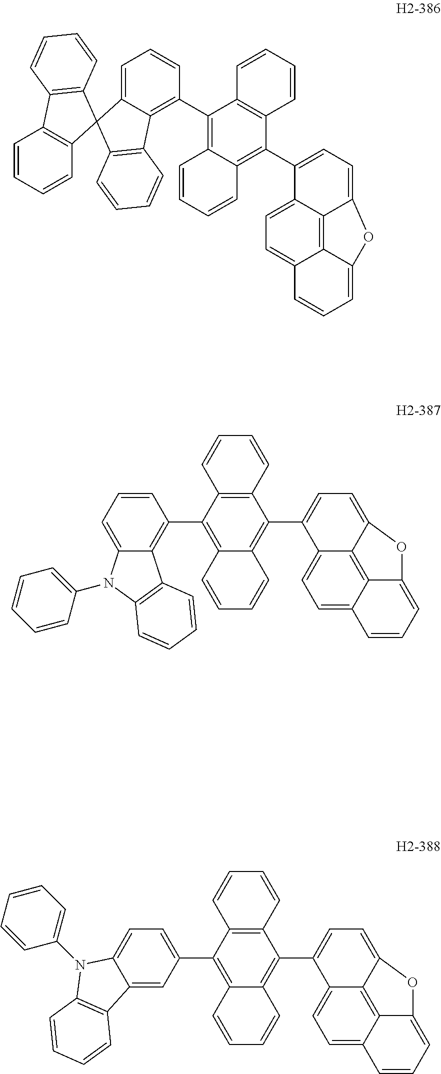 [SMILES: c1ccc2c(c1)-c1ccccc1C21c2ccccc2-c2c(-c3c4ccccc4c(-c4ccc5oc6cccc7ccc4c5c76)c4ccccc34)cccc21 ; c1ccc(-n2c3ccccc3c3c(-c4c5ccccc5c(-c5ccc6oc7cccc8ccc5c6c87)c5ccccc45)cccc32)cc1 ; c1ccc(-n2c3ccccc3c3cc(-c4c5ccccc5c(-c5ccc6oc7cccc8ccc5c6c87)c5ccccc45)ccc32)cc1] 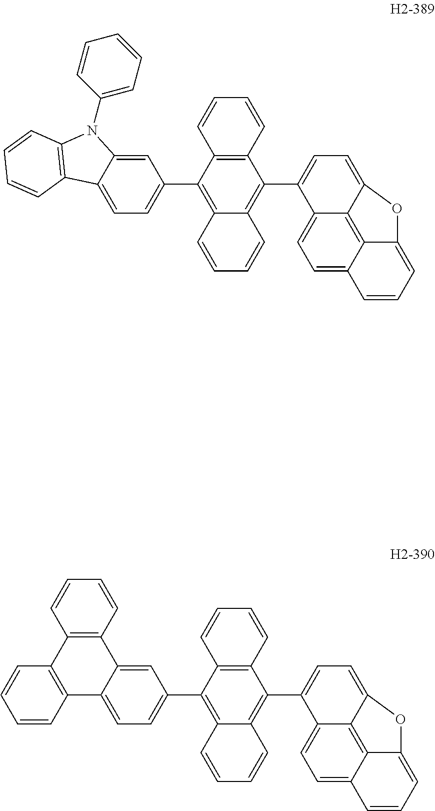 [SMILES: c1ccc(-n2c3ccccc3c3ccc(-c4c5ccccc5c(-c5ccc6oc7cccc8ccc5c6c87)c5ccccc45)cc32)cc1 ; c1cc2ccc3c(-c4c5ccccc5c(-c5ccc6c7ccccc7c7ccccc7c6c5)c5ccccc45)ccc4oc(c1)c2c43] 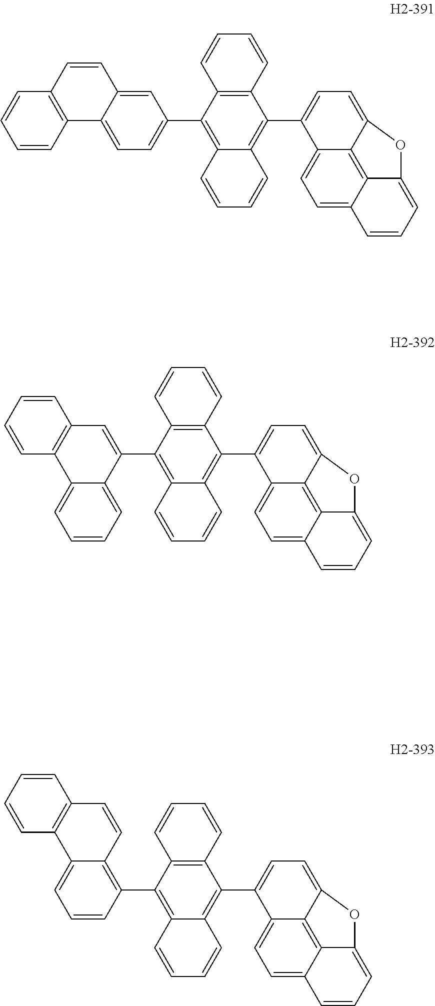 [SMILES: c1ccc2c(c1)ccc1cc(-c3c4ccccc4c(-c4ccc5oc6cccc7ccc4c5c76)c4ccccc34)ccc12 ; c1ccc2c(c1)cc(-c1c3ccccc3c(-c3ccc4oc5cccc6ccc3c4c65)c3ccccc13)c1ccccc12 ; c1ccc2c(c1)ccc1c(-c3c4ccccc4c(-c4ccc5oc6cccc7ccc4c5c76)c4ccccc34)cccc12] 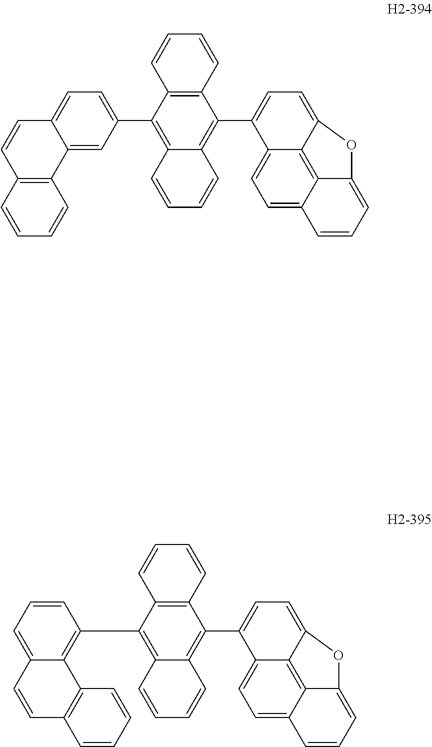 [SMILES: c1ccc2c(c1)ccc1ccc(-c3c4ccccc4c(-c4ccc5oc6cccc7ccc4c5c76)c4ccccc34)cc12 ; c1ccc2c(c1)ccc1cccc(-c3c4ccccc4c(-c4ccc5oc6cccc7ccc4c5c76)c4ccccc34)c12] 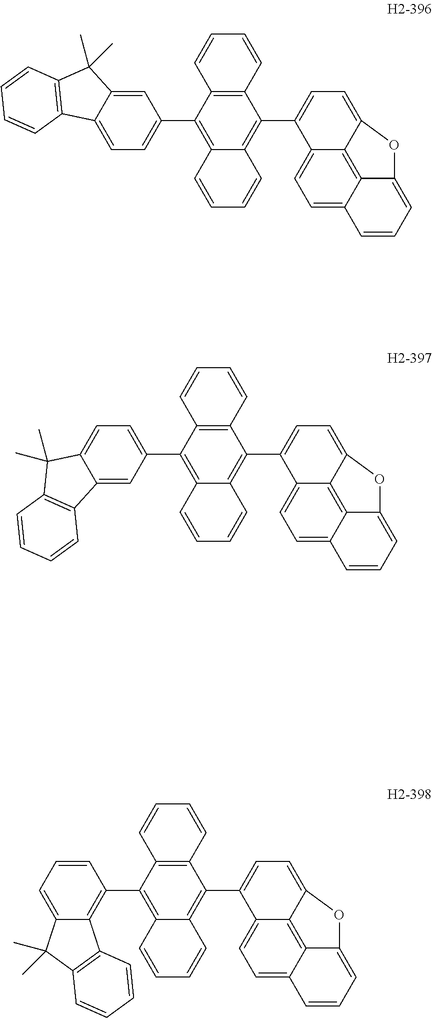 [SMILES: CC1(C)c2ccccc2-c2ccc(-c3c4ccccc4c(-c4ccc5oc6cccc7ccc4c5c76)c4ccccc34)cc21 ; CC1(C)c2ccccc2-c2cc(-c3c4ccccc4c(-c4ccc5oc6cccc7ccc4c5c76)c4ccccc34)ccc21 ; CC1(C)c2ccccc2-c2c(-c3c4ccccc4c(-c4ccc5oc6cccc7ccc4c5c76)c4ccccc34)cccc21] 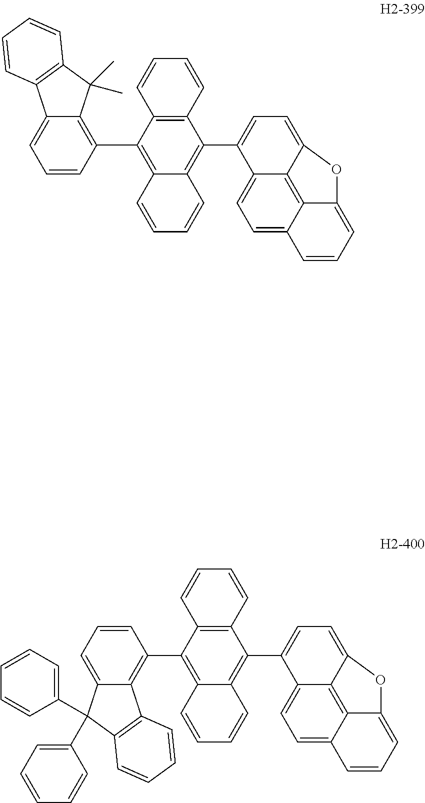 [SMILES: CC1(C)c2ccccc2-c2cccc(-c3c4ccccc4c(-c4ccc5oc6cccc7ccc4c5c76)c4ccccc34)c21 ; c1ccc(C2(c3ccccc3)c3ccccc3-c3c(-c4c5ccccc5c(-c5ccc6oc7cccc8ccc5c6c87)c5ccccc45)cccc32)cc1] 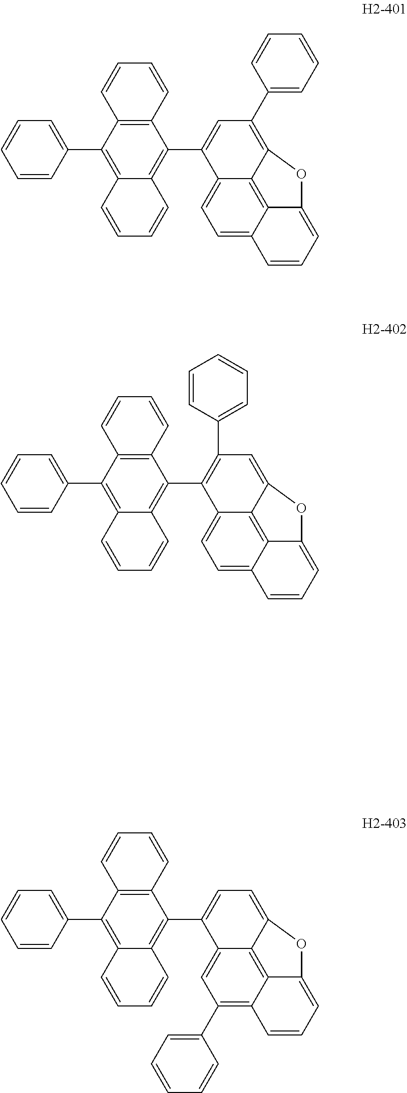 [SMILES: c1ccc(-c2c3ccccc3c(-c3cc(-c4ccccc4)c4oc5cccc6ccc3c4c65)c3ccccc23)cc1 ; c1ccc(-c2cc3oc4cccc5ccc(c2-c2c6ccccc6c(-c6ccccc6)c6ccccc26)c3c54)cc1 ; c1ccc(-c2c3ccccc3c(-c3ccc4oc5cccc6c(-c7ccccc7)cc3c4c56)c3ccccc23)cc1] 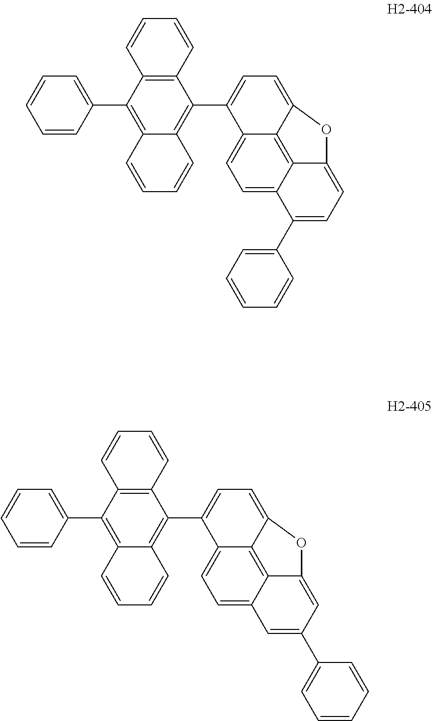 [SMILES: c1ccc(-c2c3ccccc3c(-c3ccc4oc5ccc(-c6ccccc6)c6ccc3c4c56)c3ccccc23)cc1 ; c1ccc(-c2cc3ccc4c(-c5c6ccccc6c(-c6ccccc6)c6ccccc56)ccc5oc(c2)c3c54)cc1] 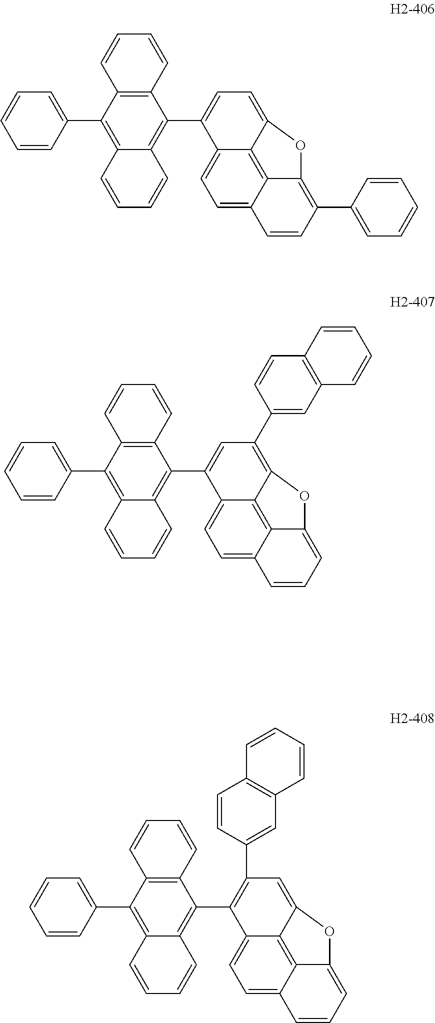 [SMILES: c1ccc(-c2c3ccccc3c(-c3ccc4oc5c(-c6ccccc6)ccc6ccc3c4c65)c3ccccc23)cc1 ; c1ccc(-c2c3ccccc3c(-c3cc(-c4ccc5ccccc5c4)c4oc5cccc6ccc3c4c65)c3ccccc23)cc1 ; c1ccc(-c2c3ccccc3c(-c3c(-c4ccc5ccccc5c4)cc4oc5cccc6ccc3c4c65)c3ccccc23)cc1] 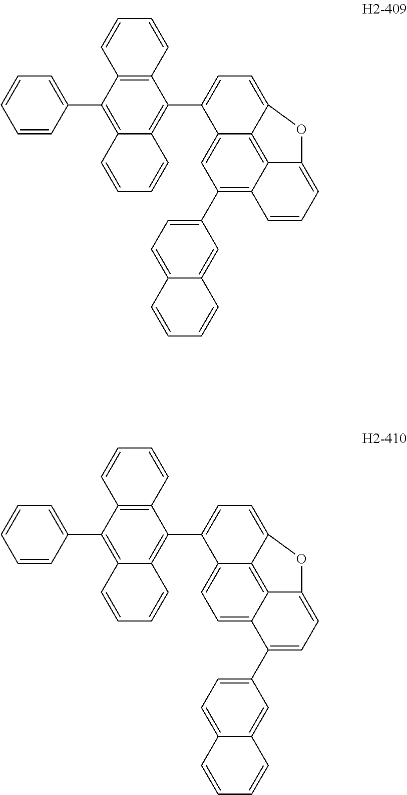 [SMILES: c1ccc(-c2c3ccccc3c(-c3ccc4oc5cccc6c(-c7ccc8ccccc8c7)cc3c4c56)c3ccccc23)cc1 ; c1ccc(-c2c3ccccc3c(-c3ccc4oc5ccc(-c6ccc7ccccc7c6)c6ccc3c4c56)c3ccccc23)cc1] 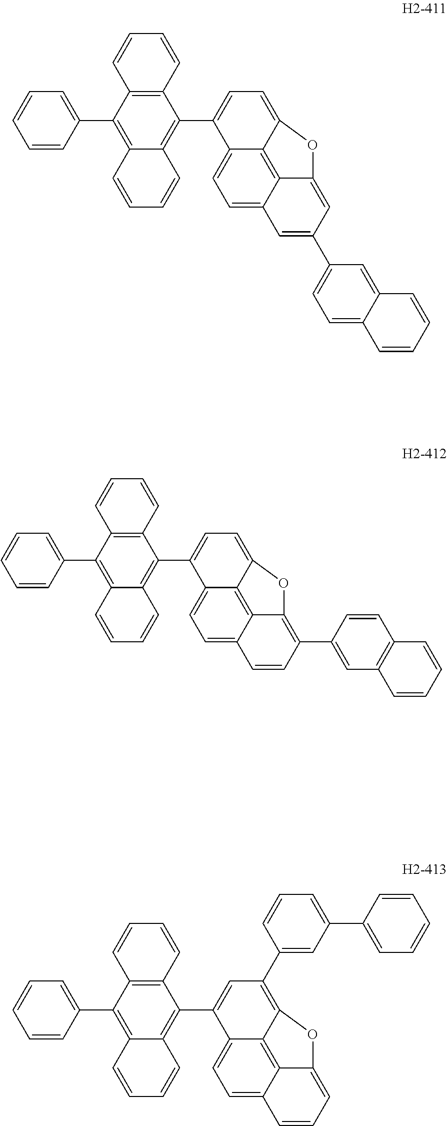 [SMILES: c1ccc(-c2c3ccccc3c(-c3ccc4oc5cc(-c6ccc7ccccc7c6)cc6ccc3c4c65)c3ccccc23)cc1 ; c1ccc(-c2c3ccccc3c(-c3ccc4oc5c(-c6ccc7ccccc7c6)ccc6ccc3c4c65)c3ccccc23)cc1 ; c1ccc(-c2cccc(-c3cc(-c4c5ccccc5c(-c5ccccc5)c5ccccc45)c4ccc5cccc6oc3c4c56)c2)cc1] 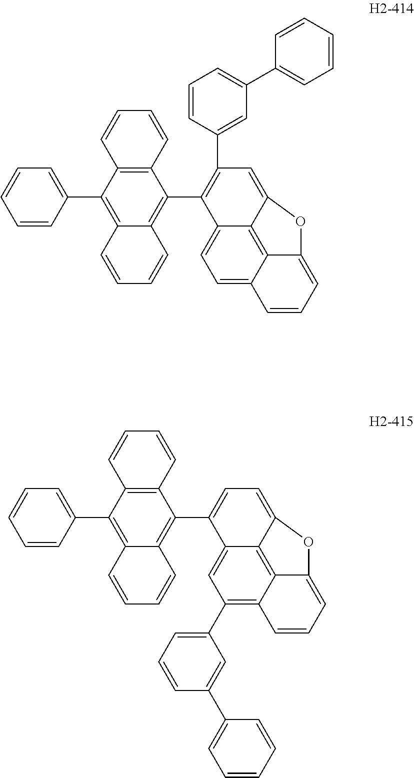 [SMILES: c1ccc(-c2cccc(-c3cc4oc5cccc6ccc(c3-c3c7ccccc7c(-c7ccccc7)c7ccccc37)c4c65)c2)cc1 ; c1ccc(-c2cccc(-c3cc4c(-c5c6ccccc6c(-c6ccccc6)c6ccccc56)ccc5oc6cccc3c6c54)c2)cc1] 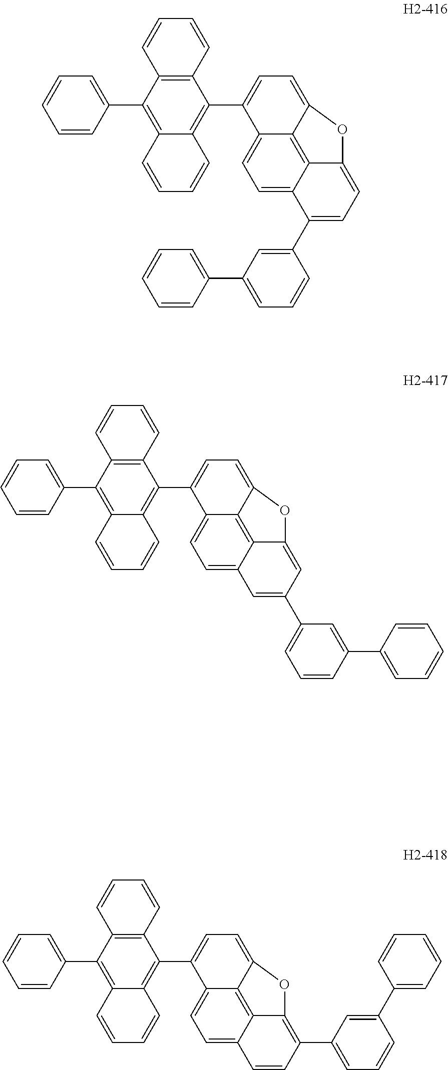 [SMILES: c1ccc(-c2cccc(-c3ccc4oc5ccc(-c6c7ccccc7c(-c7ccccc7)c7ccccc67)c6ccc3c4c56)c2)cc1 ; c1ccc(-c2cccc(-c3cc4ccc5c(-c6c7ccccc7c(-c7ccccc7)c7ccccc67)ccc6oc(c3)c4c65)c2)cc1 ; c1ccc(-c2cccc(-c3ccc4ccc5c(-c6c7ccccc7c(-c7ccccc7)c7ccccc67)ccc6oc3c4c65)c2)cc1] 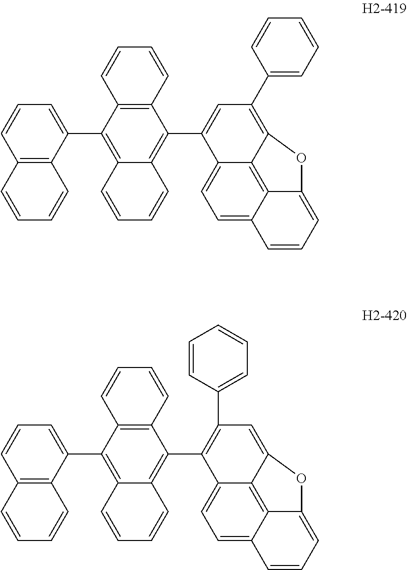 [SMILES: c1ccc(-c2cc(-c3c4ccccc4c(-c4cccc5ccccc45)c4ccccc34)c3ccc4cccc5oc2c3c45)cc1 ; c1ccc(-c2cc3oc4cccc5ccc(c2-c2c6ccccc6c(-c6cccc7ccccc67)c6ccccc26)c3c54)cc1] 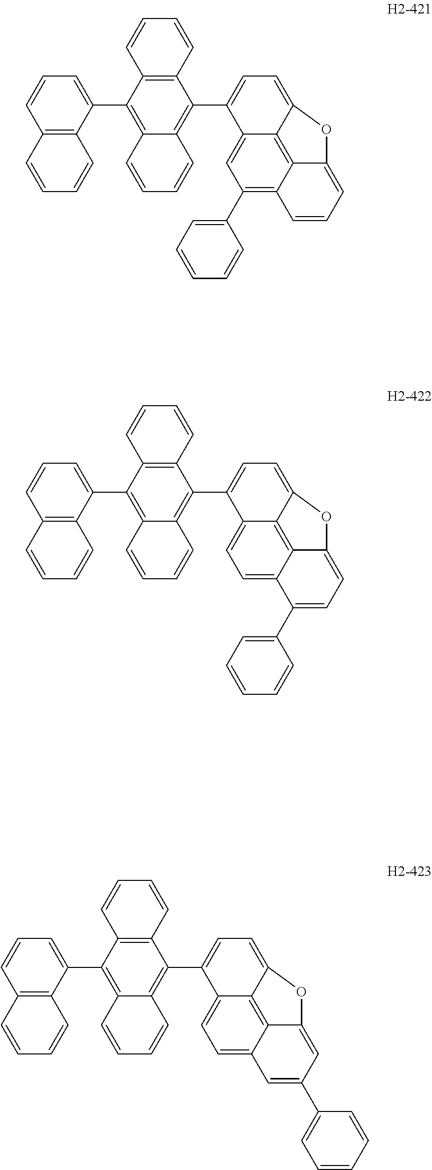 [SMILES: c1ccc(-c2cc3c(-c4c5ccccc5c(-c5cccc6ccccc56)c5ccccc45)ccc4oc5cccc2c5c43)cc1 ; c1ccc(-c2ccc3oc4ccc(-c5c6ccccc6c(-c6cccc7ccccc67)c6ccccc56)c5ccc2c3c45)cc1 ; c1ccc(-c2cc3ccc4c(-c5c6ccccc6c(-c6cccc7ccccc67)c6ccccc56)ccc5oc(c2)c3c54)cc1] 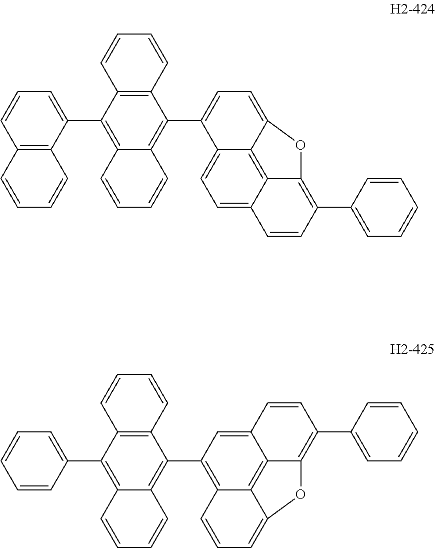 [SMILES: c1ccc(-c2ccc3ccc4c(-c5c6ccccc6c(-c6cccc7ccccc67)c6ccccc56)ccc5oc2c3c54)cc1 ; c1ccc(-c2c3ccccc3c(-c3cc4ccc(-c5ccccc5)c5oc6cccc3c6c45)c3ccccc23)cc1] 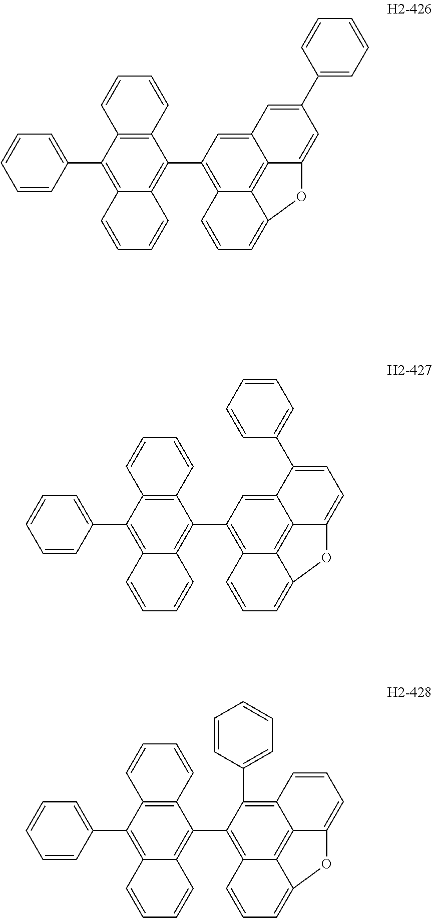 [SMILES: c1ccc(-c2cc3cc(-c4c5ccccc5c(-c5ccccc5)c5ccccc45)c4cccc5oc(c2)c3c54)cc1 ; c1ccc(-c2c3ccccc3c(-c3cc4c(-c5ccccc5)ccc5oc6cccc3c6c54)c3ccccc23)cc1 ; c1ccc(-c2c3ccccc3c(-c3c(-c4ccccc4)c4cccc5oc6cccc3c6c54)c3ccccc23)cc1] 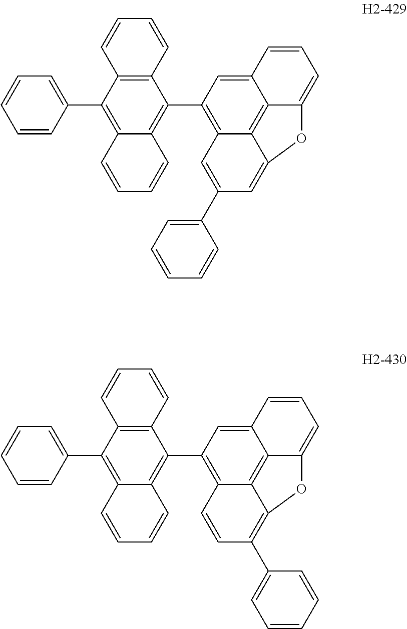 [SMILES: c1ccc(-c2cc3oc4cccc5cc(-c6c7ccccc7c(-c7ccccc7)c7ccccc67)c(c2)c3c54)cc1 ; c1ccc(-c2c3ccccc3c(-c3cc4cccc5oc6c(-c7ccccc7)ccc3c6c45)c3ccccc23)cc1] 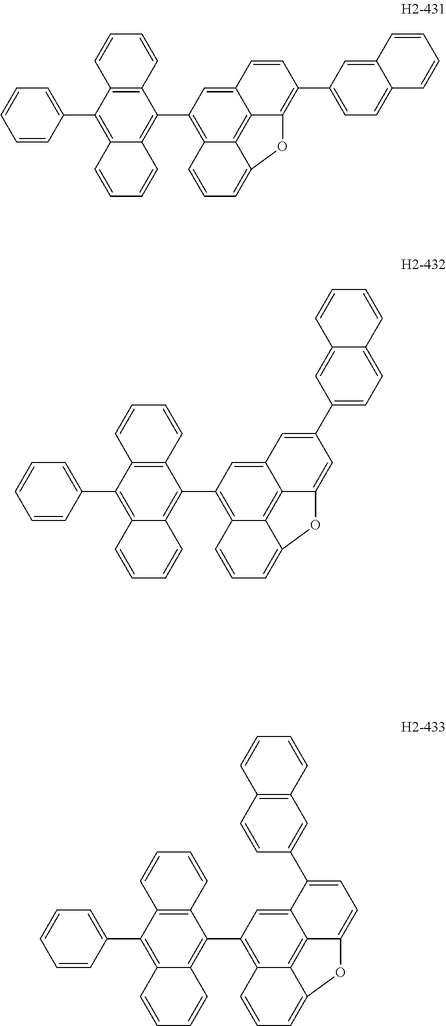 [SMILES: c1ccc(-c2c3ccccc3c(-c3cc4ccc(-c5ccc6ccccc6c5)c5oc6cccc3c6c45)c3ccccc23)cc1 ; c1ccc(-c2c3ccccc3c(-c3cc4cc(-c5ccc6ccccc6c5)cc5oc6cccc3c6c45)c3ccccc23)cc1 ; c1ccc(-c2c3ccccc3c(-c3cc4c(-c5ccc6ccccc6c5)ccc5oc6cccc3c6c54)c3ccccc23)cc1] 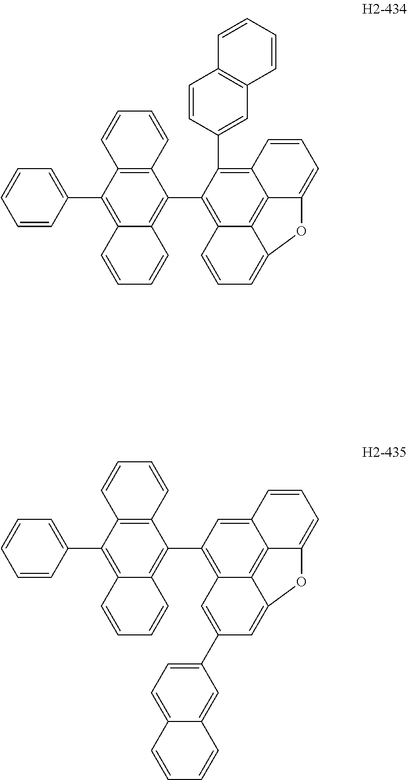 [SMILES: c1ccc(-c2c3ccccc3c(-c3c(-c4ccc5ccccc5c4)c4cccc5oc6cccc3c6c54)c3ccccc23)cc1 ; c1ccc(-c2c3ccccc3c(-c3cc4cccc5oc6cc(-c7ccc8ccccc8c7)cc3c6c45)c3ccccc23)cc1] 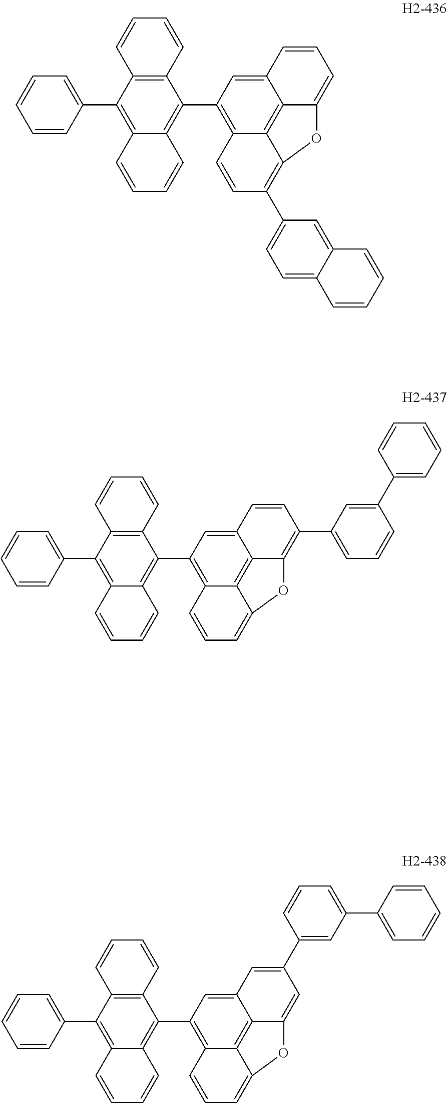 [SMILES: c1ccc(-c2c3ccccc3c(-c3cc4cccc5oc6c(-c7ccc8ccccc8c7)ccc3c6c45)c3ccccc23)cc1 ; c1ccc(-c2cccc(-c3ccc4cc(-c5c6ccccc6c(-c6ccccc6)c6ccccc56)c5cccc6oc3c4c65)c2)cc1 ; c1ccc(-c2cccc(-c3cc4cc(-c5c6ccccc6c(-c6ccccc6)c6ccccc56)c5cccc6oc(c3)c4c65)c2)cc1] 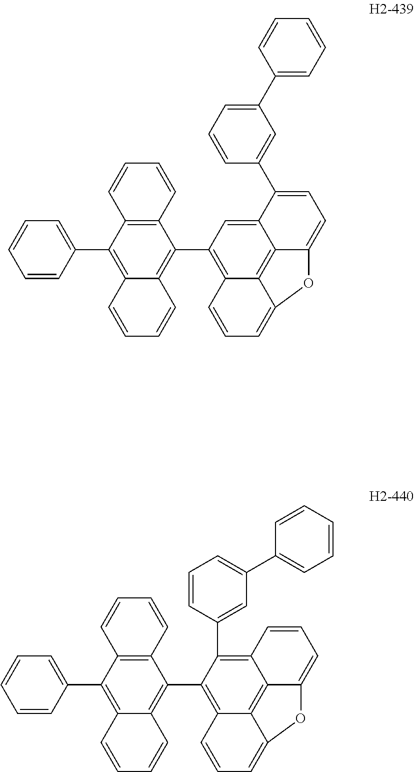 [SMILES: c1ccc(-c2cccc(-c3ccc4oc5cccc6c(-c7c8ccccc8c(-c8ccccc8)c8ccccc78)cc3c4c56)c2)cc1 ; c1ccc(-c2cccc(-c3c(-c4c5ccccc5c(-c5ccccc5)c5ccccc45)c4cccc5oc6cccc3c6c54)c2)cc1] 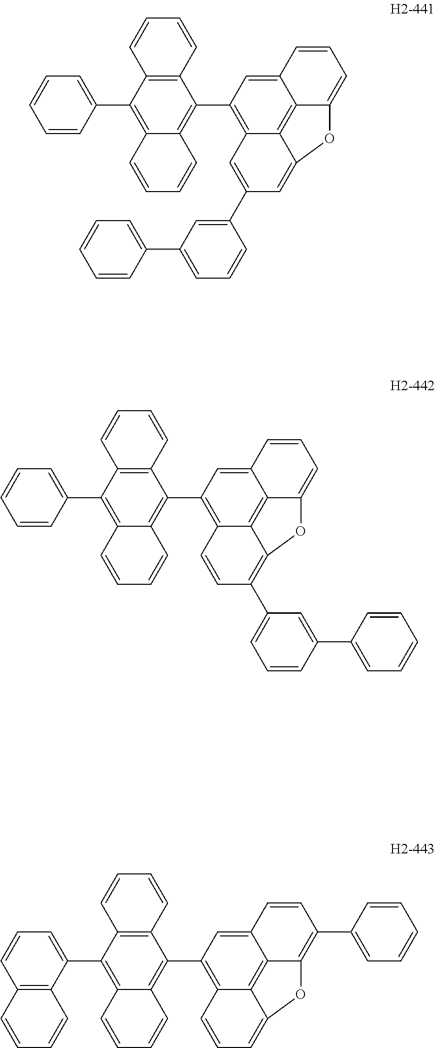 [SMILES: c1ccc(-c2cccc(-c3cc4oc5cccc6cc(-c7c8ccccc8c(-c8ccccc8)c8ccccc78)c(c3)c4c65)c2)cc1 ; c1ccc(-c2cccc(-c3ccc4c(-c5c6ccccc6c(-c6ccccc6)c6ccccc56)cc5cccc6oc3c4c56)c2)cc1 ; c1ccc(-c2ccc3cc(-c4c5ccccc5c(-c5cccc6ccccc56)c5ccccc45)c4cccc5oc2c3c54)cc1] 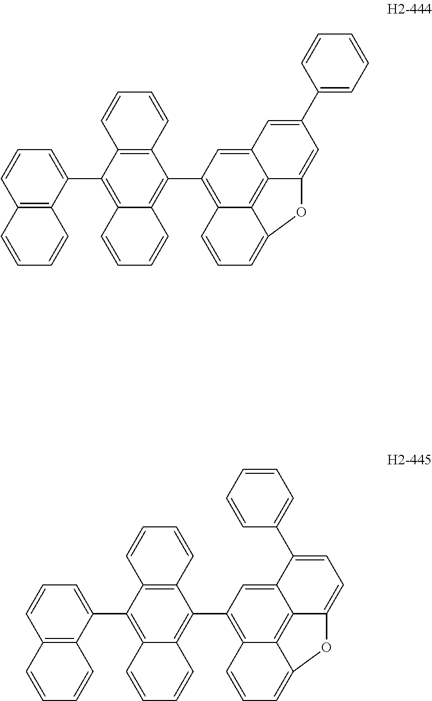 [SMILES: c1ccc(-c2cc3cc(-c4c5ccccc5c(-c5cccc6ccccc56)c5ccccc45)c4cccc5oc(c2)c3c54)cc1 ; c1ccc(-c2ccc3oc4cccc5c(-c6c7ccccc7c(-c7cccc8ccccc78)c7ccccc67)cc2c3c45)cc1] 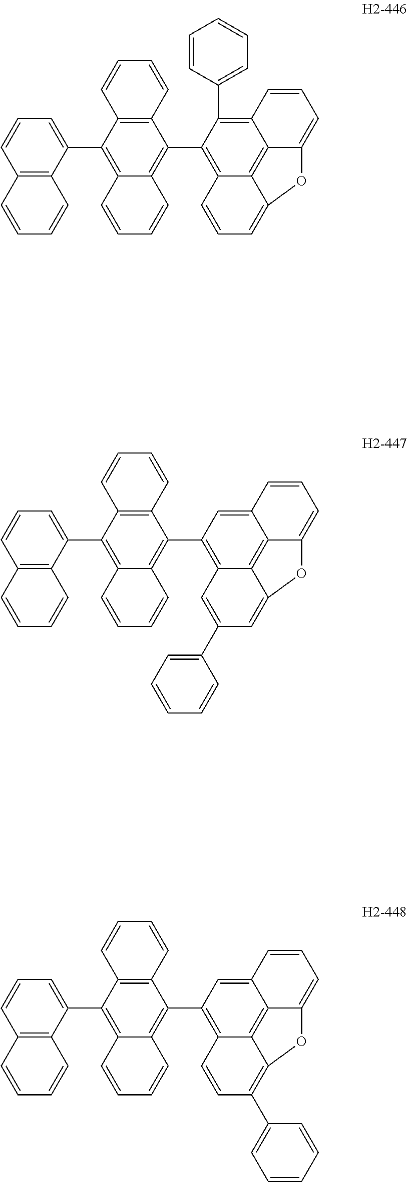 [SMILES: c1ccc(-c2c(-c3c4ccccc4c(-c4cccc5ccccc45)c4ccccc34)c3cccc4oc5cccc2c5c43)cc1 ; c1ccc(-c2cc3oc4cccc5cc(-c6c7ccccc7c(-c7cccc8ccccc78)c7ccccc67)c(c2)c3c54)cc1 ; c1ccc(-c2ccc3c(-c4c5ccccc5c(-c5cccc6ccccc56)c5ccccc45)cc4cccc5oc2c3c45)cc1] 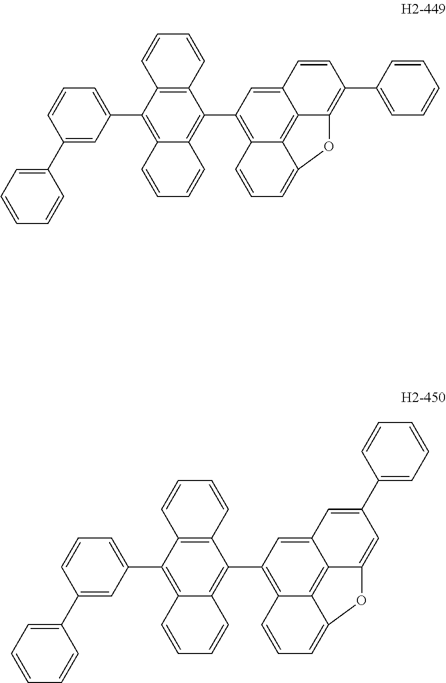 [SMILES: c1ccc(-c2cccc(-c3c4ccccc4c(-c4cc5ccc(-c6ccccc6)c6oc7cccc4c7c56)c4ccccc34)c2)cc1 ; c1ccc(-c2cccc(-c3c4ccccc4c(-c4cc5cc(-c6ccccc6)cc6oc7cccc4c7c56)c4ccccc34)c2)cc1] 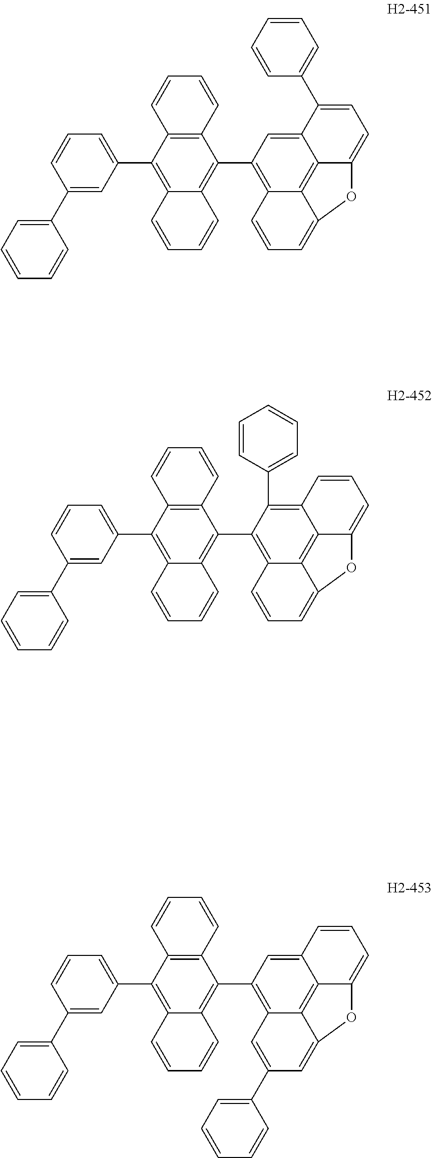 [SMILES: c1ccc(-c2cccc(-c3c4ccccc4c(-c4cc5c(-c6ccccc6)ccc6oc7cccc4c7c65)c4ccccc34)c2)cc1 ; c1ccc(-c2cccc(-c3c4ccccc4c(-c4c(-c5ccccc5)c5cccc6oc7cccc4c7c65)c4ccccc34)c2)cc1 ; c1ccc(-c2cccc(-c3c4ccccc4c(-c4cc5cccc6oc7cc(-c8ccccc8)cc4c7c56)c4ccccc34)c2)cc1] 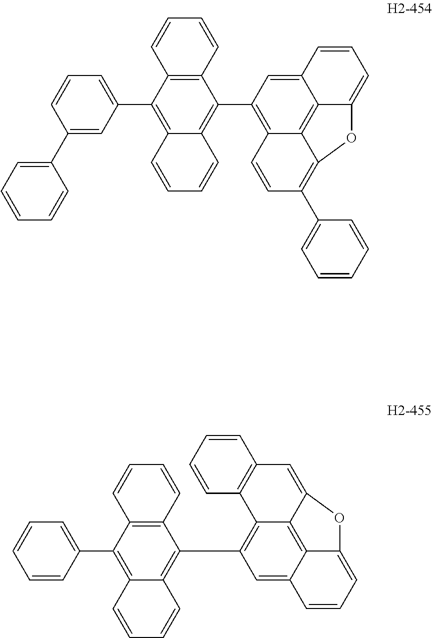 [SMILES: c1ccc(-c2cccc(-c3c4ccccc4c(-c4cc5cccc6oc7c(-c8ccccc8)ccc4c7c56)c4ccccc34)c2)cc1 ; c1ccc(-c2c3ccccc3c(-c3cc4cccc5oc6cc7ccccc7c3c6c45)c3ccccc23)cc1] 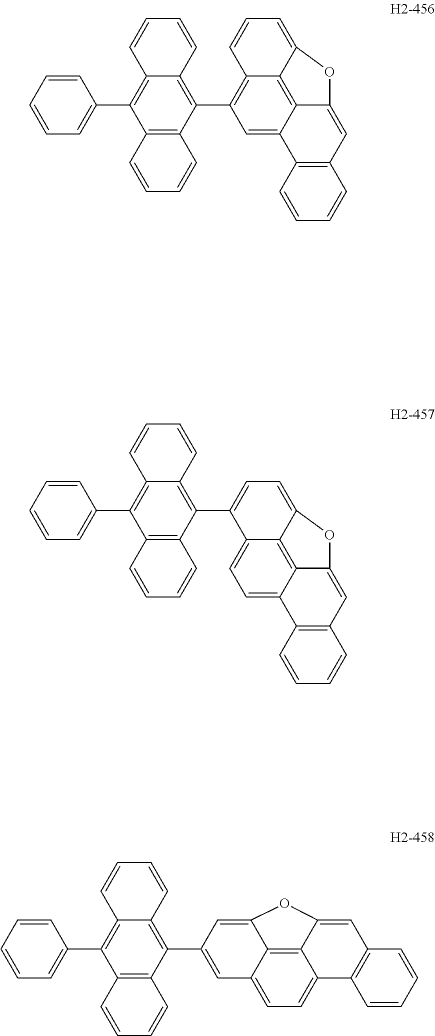 [SMILES: c1ccc(-c2c3ccccc3c(-c3cc4c5ccccc5cc5oc6cccc3c6c54)c3ccccc23)cc1 ; c1ccc(-c2c3ccccc3c(-c3ccc4oc5cc6ccccc6c6ccc3c4c56)c3ccccc23)cc1 ; c1ccc(-c2c3ccccc3c(-c3cc4ccc5c6ccccc6cc6oc(c3)c4c65)c3ccccc23)cc1] 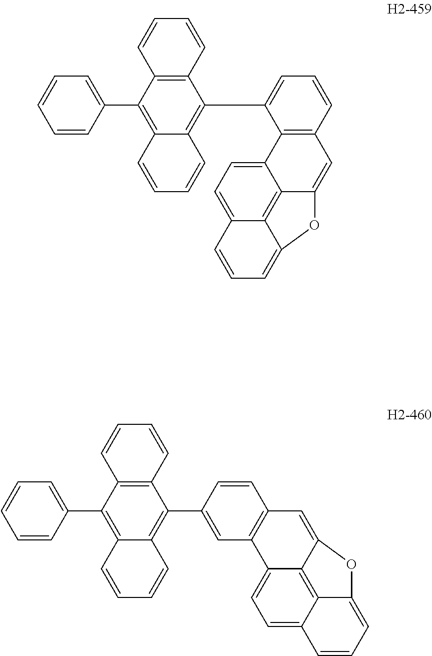 [SMILES: c1ccc(-c2c3ccccc3c(-c3cccc4cc5oc6cccc7ccc(c34)c5c76)c3ccccc23)cc1 ; c1ccc(-c2c3ccccc3c(-c3ccc4cc5oc6cccc7ccc(c4c3)c5c76)c3ccccc23)cc1] 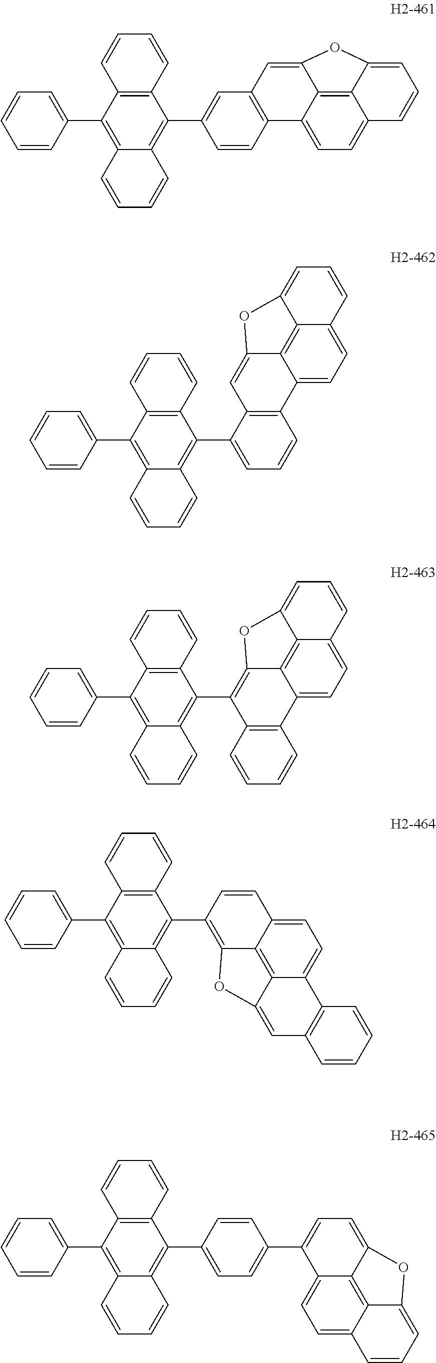 [SMILES: c1ccc(-c2c3ccccc3c(-c3ccc4c(c3)cc3oc5cccc6ccc4c3c65)c3ccccc23)cc1 ; c1ccc(-c2c3ccccc3c(-c3cccc4c3cc3oc5cccc6ccc4c3c65)c3ccccc23)cc1 ; c1ccc(-c2c3ccccc3c(-c3c4ccccc4c4ccc5cccc6oc3c4c56)c3ccccc23)cc1 ; c1ccc(-c2c3ccccc3c(-c3ccc4ccc5c6ccccc6cc6oc3c4c65)c3ccccc23)cc1 ; c1ccc(-c2c3ccccc3c(-c3ccc(-c4ccc5oc6cccc7ccc4c5c76)cc3)c3ccccc23)cc1] 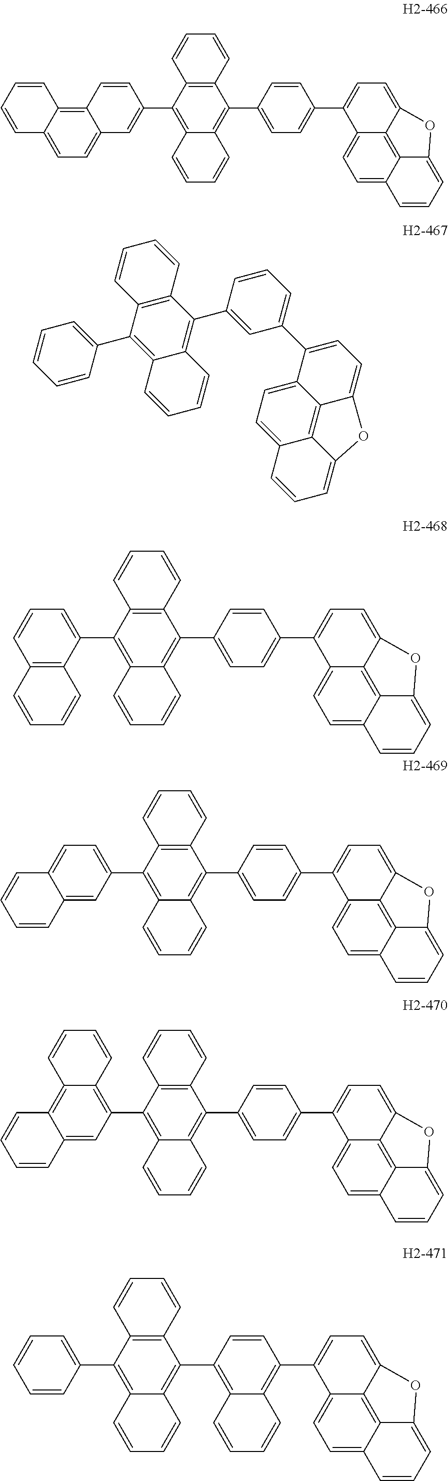 [SMILES: c1ccc2c(c1)ccc1cc(-c3c4ccccc4c(-c4ccc(-c5ccc6oc7cccc8ccc5c6c87)cc4)c4ccccc34)ccc12 ; c1ccc(-c2c3ccccc3c(-c3cccc(-c4ccc5oc6cccc7ccc4c5c76)c3)c3ccccc23)cc1 ; c1ccc2c(-c3c4ccccc4c(-c4ccc(-c5ccc6oc7cccc8ccc5c6c87)cc4)c4ccccc34)cccc2c1 ; c1ccc2cc(-c3c4ccccc4c(-c4ccc(-c5ccc6oc7cccc8ccc5c6c87)cc4)c4ccccc34)ccc2c1 ; c1ccc2c(c1)cc(-c1c3ccccc3c(-c3ccc(-c4ccc5oc6cccc7ccc4c5c76)cc3)c3ccccc13)c1ccccc12 ; c1ccc(-c2c3ccccc3c(-c3ccc(-c4ccc5oc6cccc7ccc4c5c76)c4ccccc34)c3ccccc23)cc1] 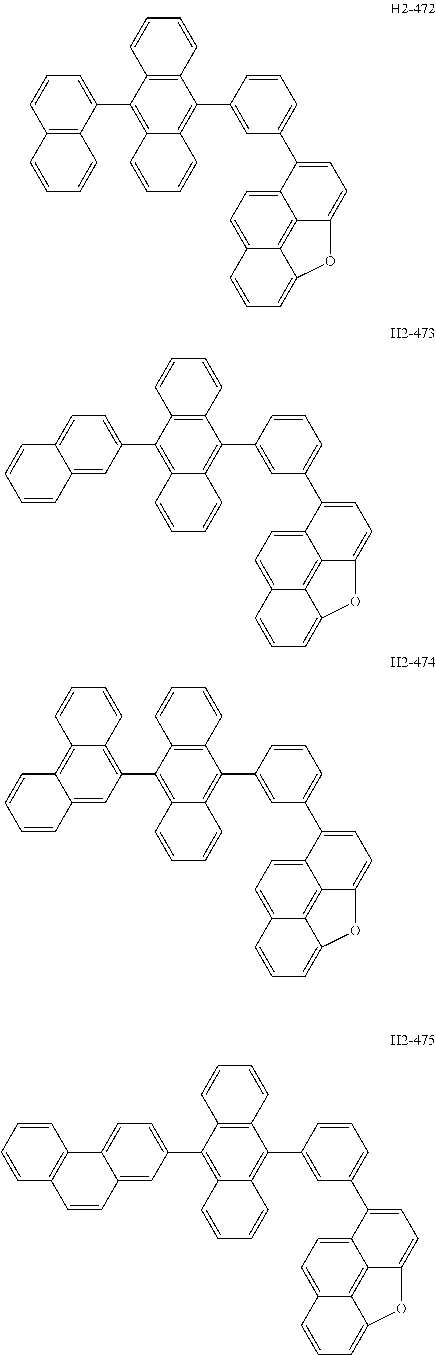 [SMILES: c1cc(-c2c3ccccc3c(-c3cccc4ccccc34)c3ccccc23)cc(-c2ccc3oc4cccc5ccc2c3c54)c1 ; c1cc(-c2c3ccccc3c(-c3ccc4ccccc4c3)c3ccccc23)cc(-c2ccc3oc4cccc5ccc2c3c54)c1 ; c1cc(-c2c3ccccc3c(-c3cc4ccccc4c4ccccc34)c3ccccc23)cc(-c2ccc3oc4cccc5ccc2c3c54)c1 ; c1cc(-c2c3ccccc3c(-c3ccc4c(ccc5ccccc54)c3)c3ccccc23)cc(-c2ccc3oc4cccc5ccc2c3c54)c1] 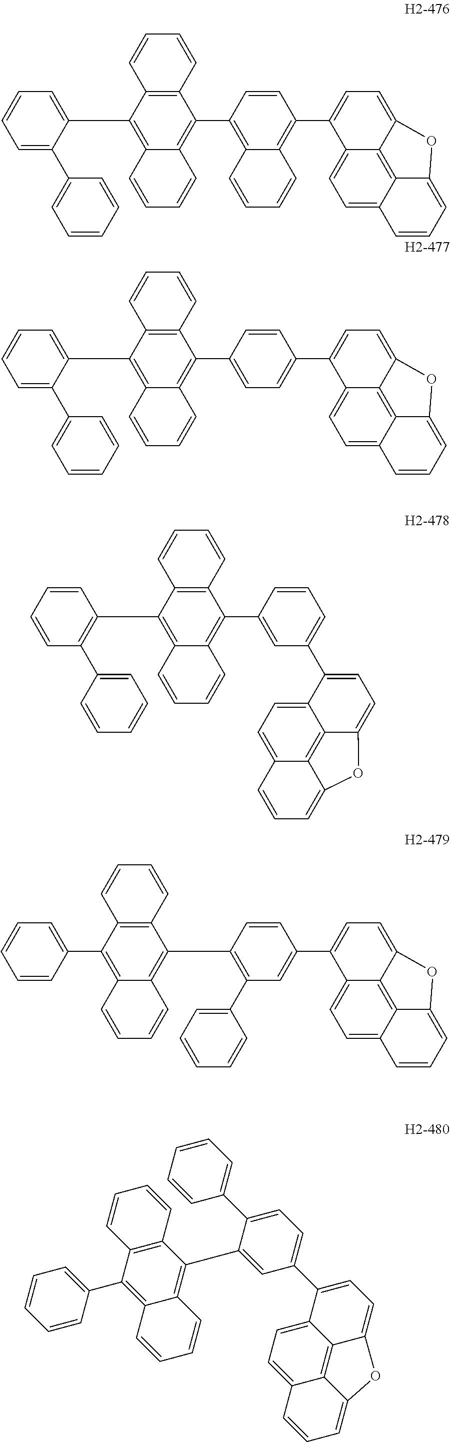 [SMILES: c1ccc(-c2ccccc2-c2c3ccccc3c(-c3ccc(-c4ccc5oc6cccc7ccc4c5c76)c4ccccc34)c3ccccc23)cc1 ; c1ccc(-c2ccccc2-c2c3ccccc3c(-c3ccc(-c4ccc5oc6cccc7ccc4c5c76)cc3)c3ccccc23)cc1 ; c1ccc(-c2ccccc2-c2c3ccccc3c(-c3cccc(-c4ccc5oc6cccc7ccc4c5c76)c3)c3ccccc23)cc1 ; c1ccc(-c2cc(-c3ccc4oc5cccc6ccc3c4c65)ccc2-c2c3ccccc3c(-c3ccccc3)c3ccccc23)cc1 ; c1ccc(-c2ccc(-c3ccc4oc5cccc6ccc3c4c65)cc2-c2c3ccccc3c(-c3ccccc3)c3ccccc23)cc1] 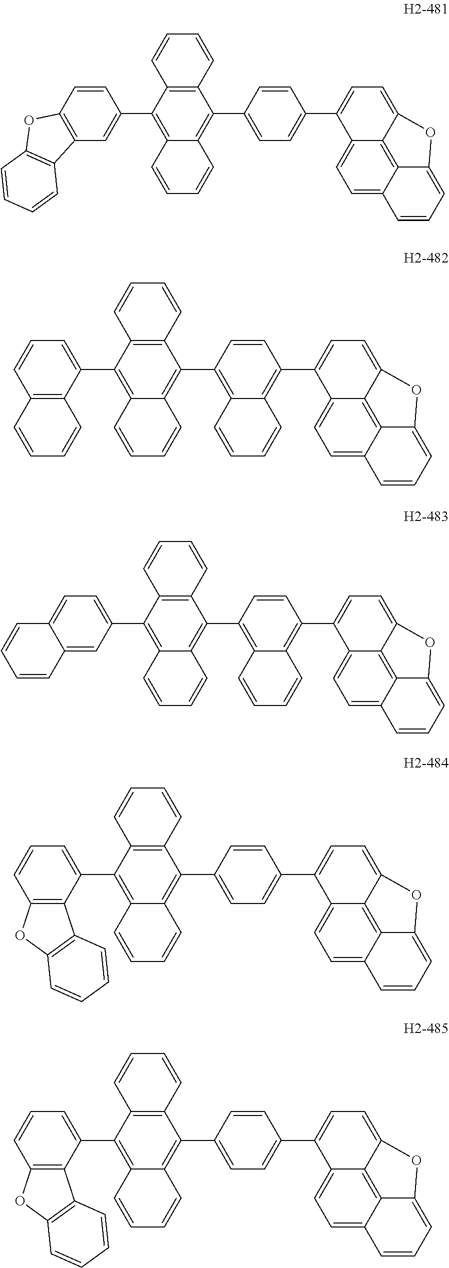 [SMILES: c1ccc2c(c1)oc1ccc(-c3c4ccccc4c(-c4ccc(-c5ccc6oc7cccc8ccc5c6c87)cc4)c4ccccc34)cc12 ; c1ccc2c(-c3c4ccccc4c(-c4ccc(-c5ccc6oc7cccc8ccc5c6c87)c5ccccc45)c4ccccc34)cccc2c1 ; c1ccc2cc(-c3c4ccccc4c(-c4ccc(-c5ccc6oc7cccc8ccc5c6c87)c5ccccc45)c4ccccc34)ccc2c1 ; c1ccc2c(c1)oc1cccc(-c3c4ccccc4c(-c4ccc(-c5ccc6oc7cccc8ccc5c6c87)cc4)c4ccccc34)c12 ; c1ccc2c(c1)oc1cccc(-c3c4ccccc4c(-c4ccc(-c5ccc6oc7cccc8ccc5c6c87)cc4)c4ccccc34)c12] 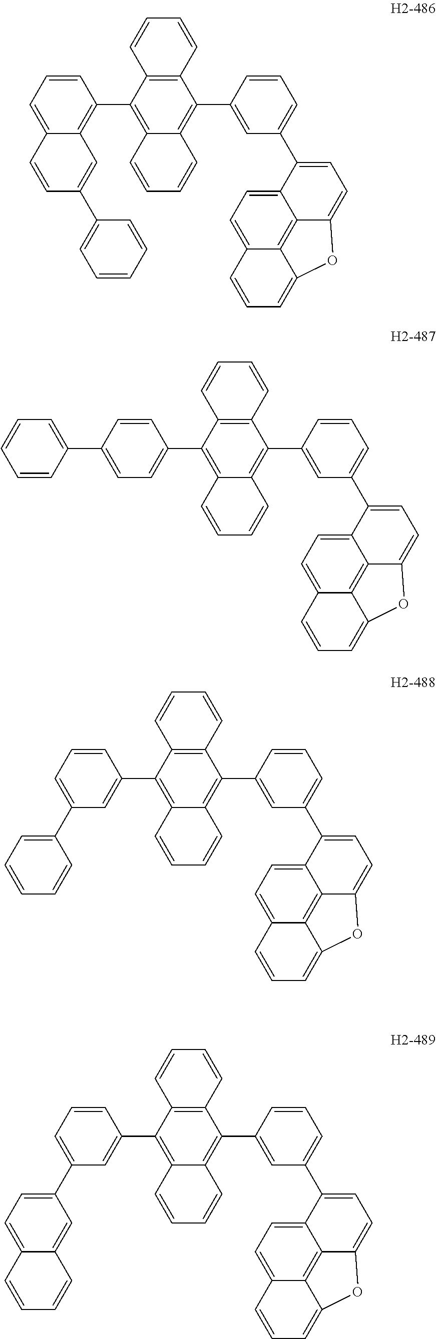 [SMILES: c1ccc(-c2ccc3cccc(-c4c5ccccc5c(-c5cccc(-c6ccc7oc8cccc9ccc6c7c98)c5)c5ccccc45)c3c2)cc1 ; c1ccc(-c2ccc(-c3c4ccccc4c(-c4cccc(-c5ccc6oc7cccc8ccc5c6c87)c4)c4ccccc34)cc2)cc1 ; c1ccc(-c2cccc(-c3c4ccccc4c(-c4cccc(-c5ccc6oc7cccc8ccc5c6c87)c4)c4ccccc34)c2)cc1 ; c1cc(-c2ccc3ccccc3c2)cc(-c2c3ccccc3c(-c3cccc(-c4ccc5oc6cccc7ccc4c5c76)c3)c3ccccc23)c1] 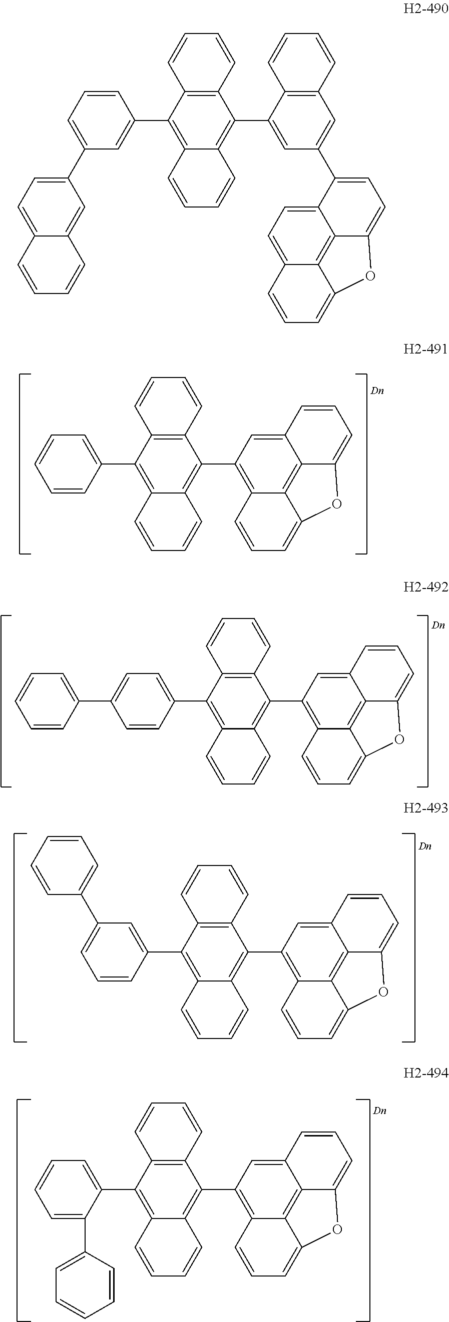 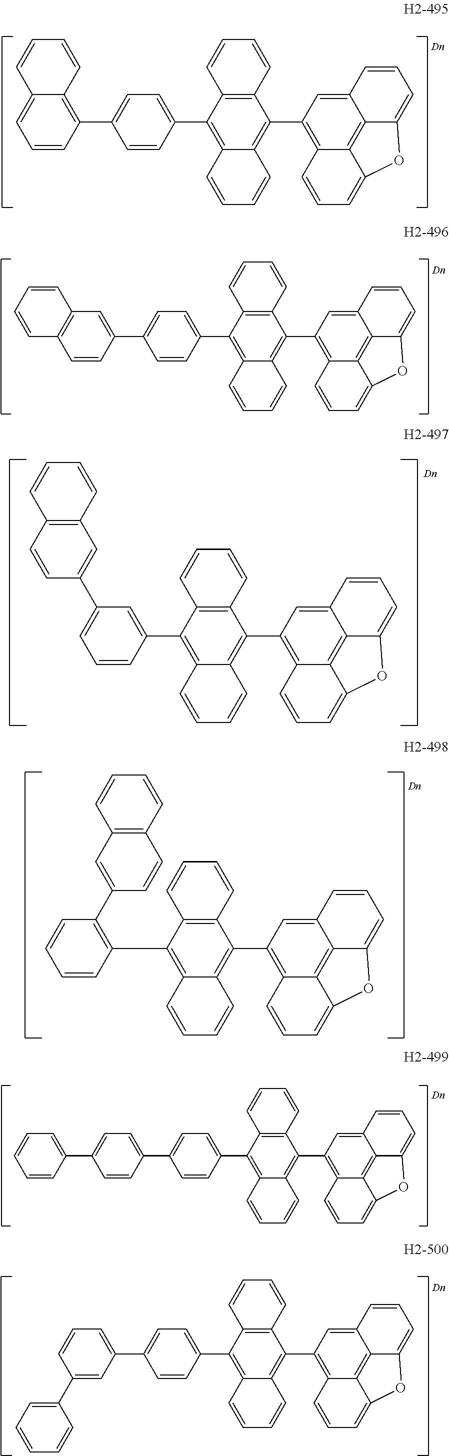 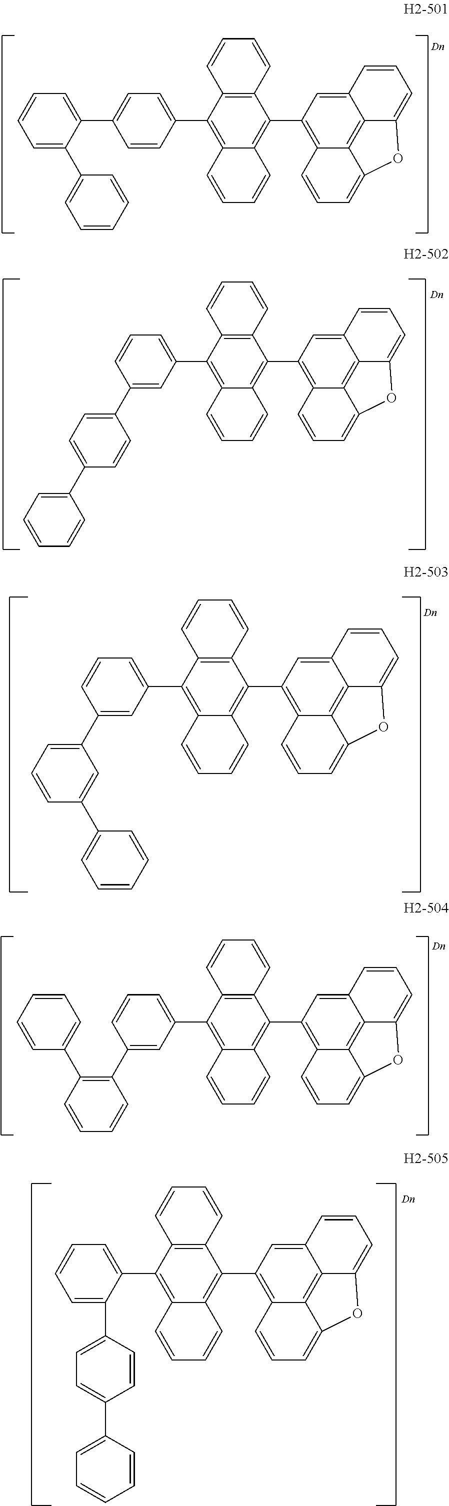 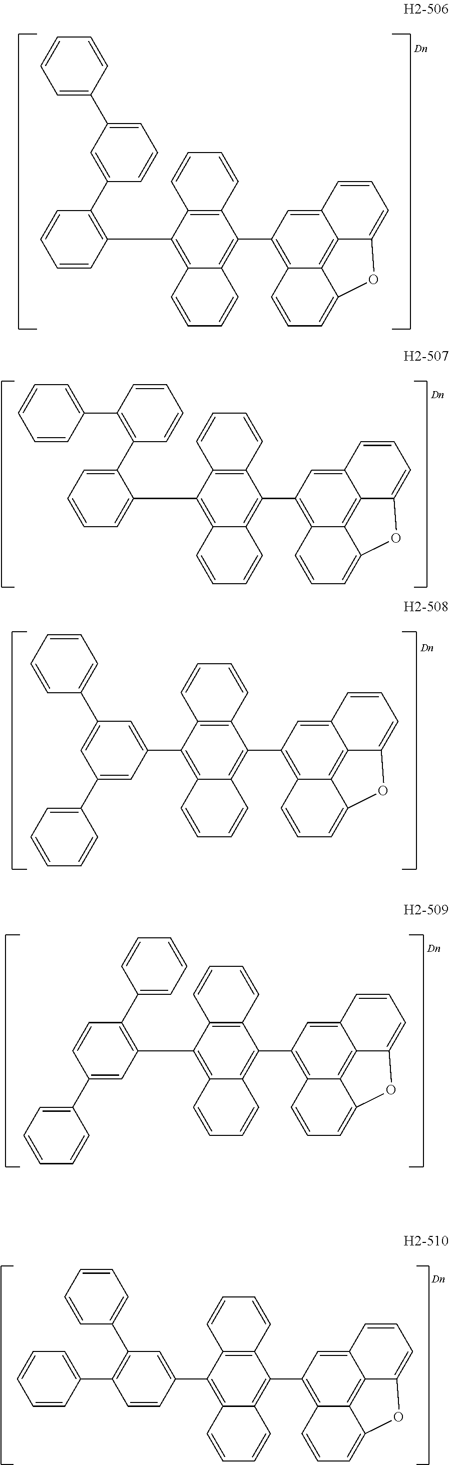 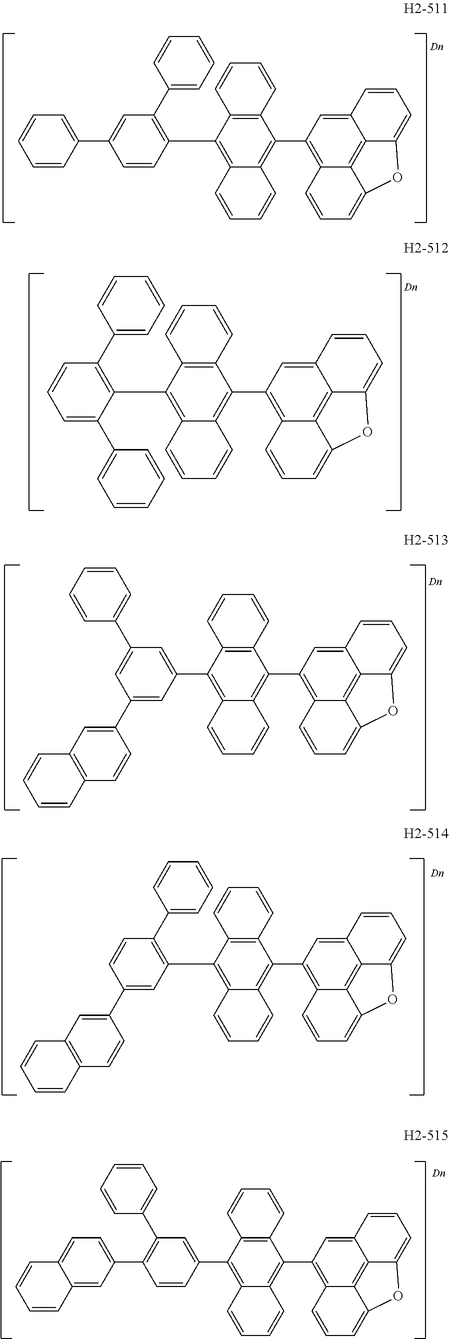 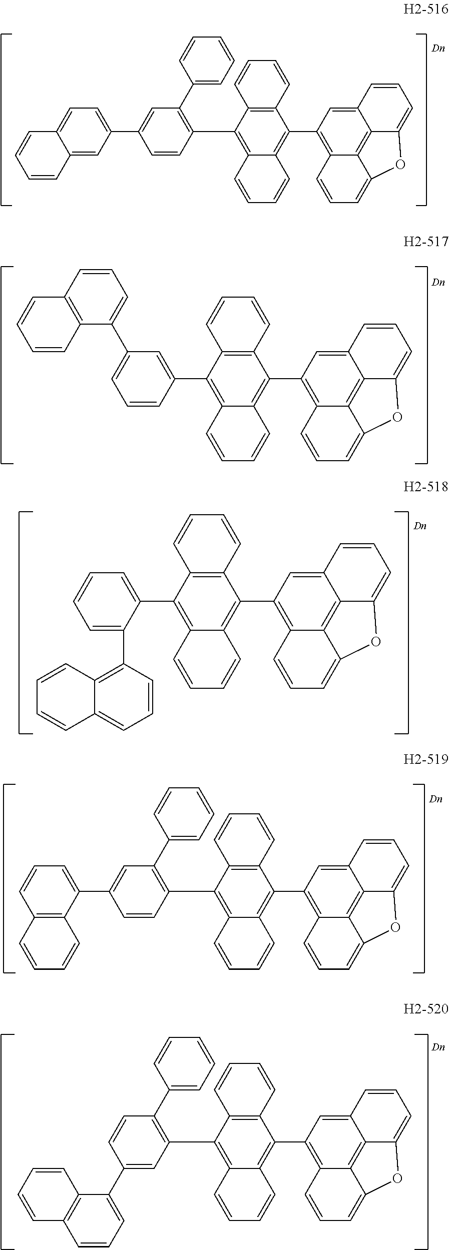 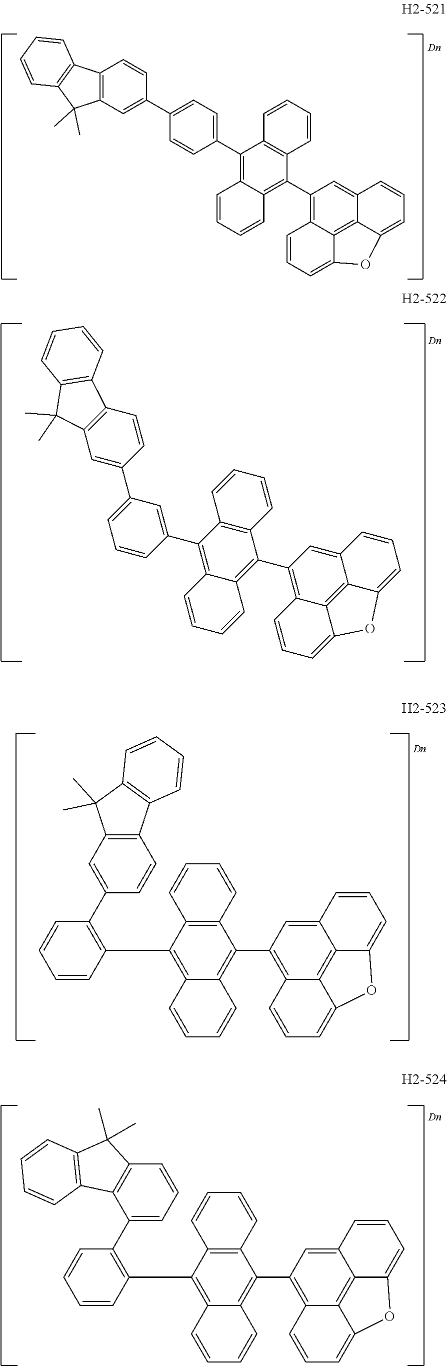 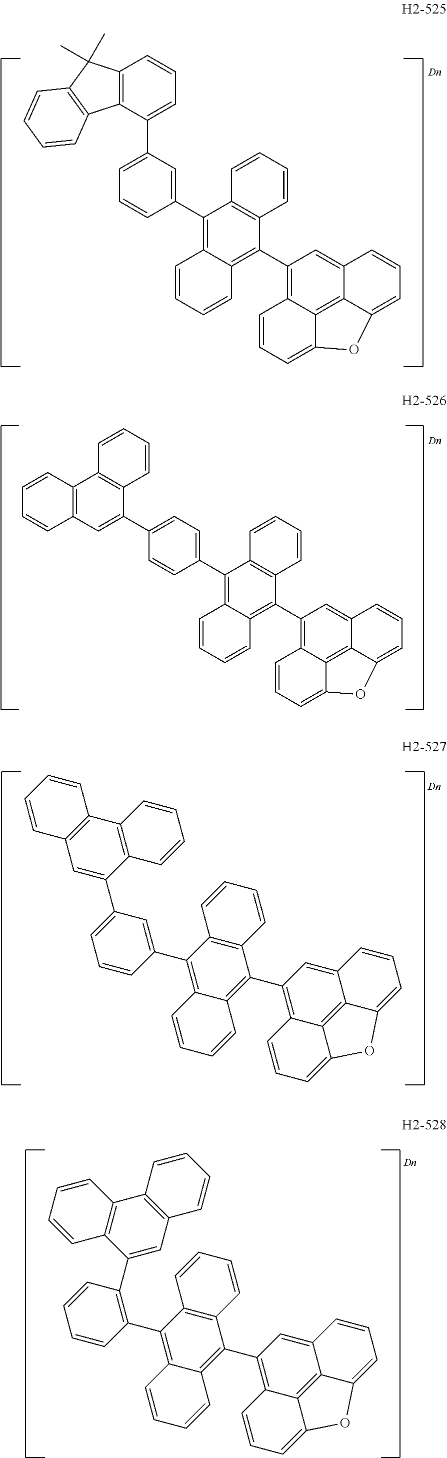 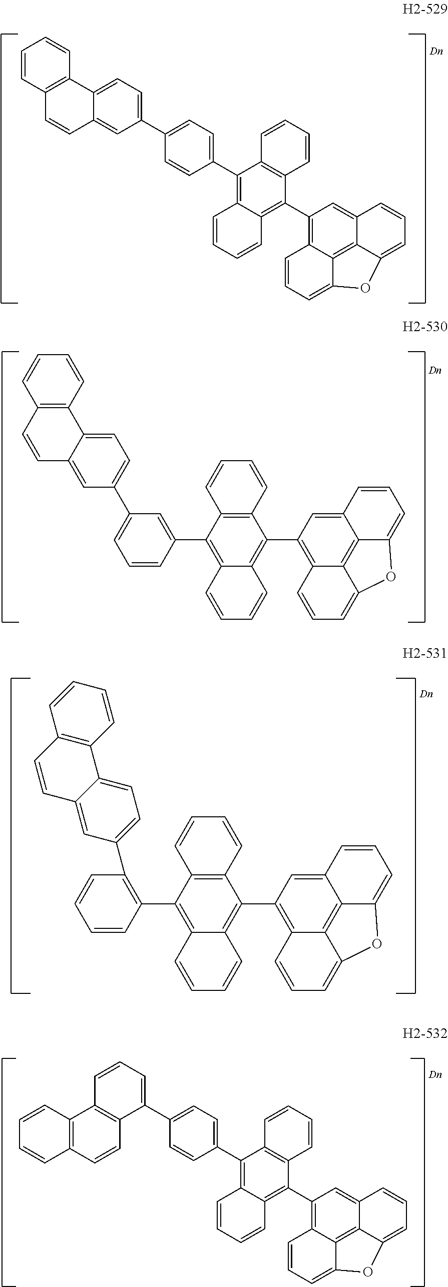 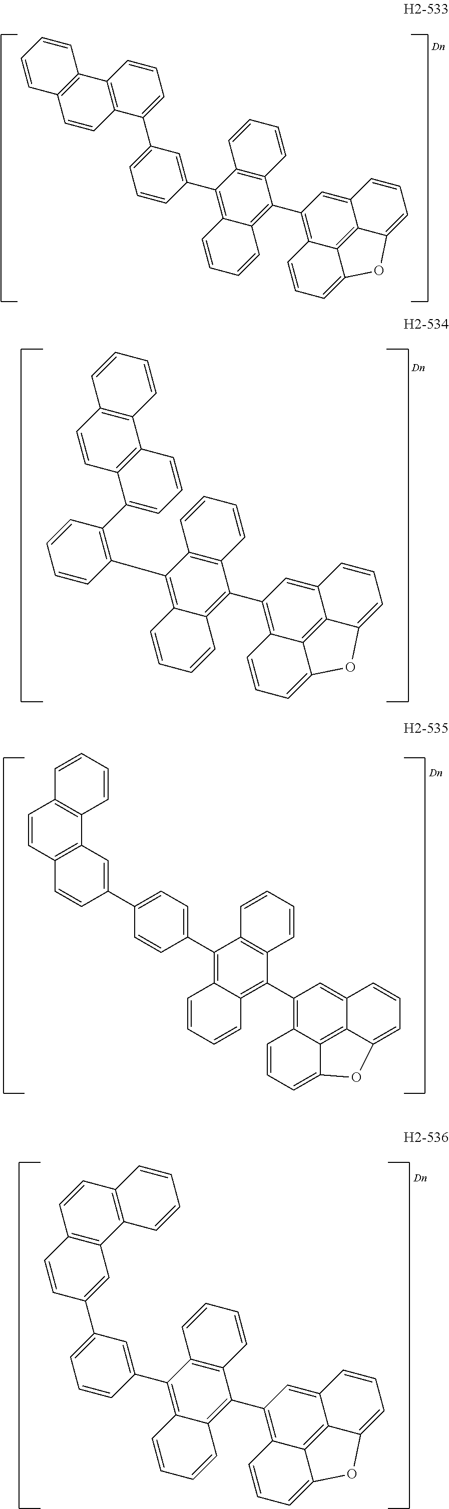 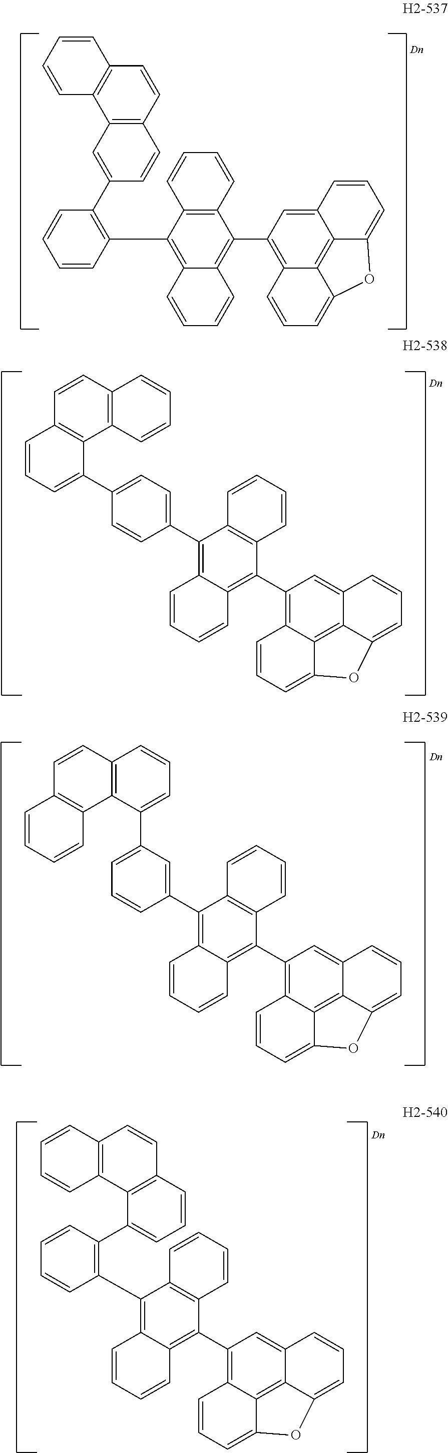 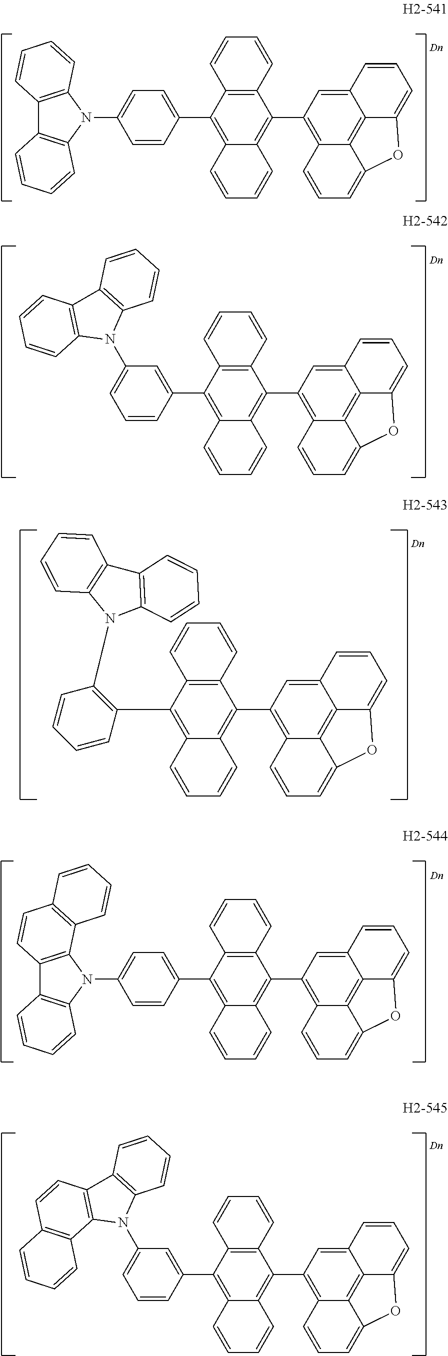 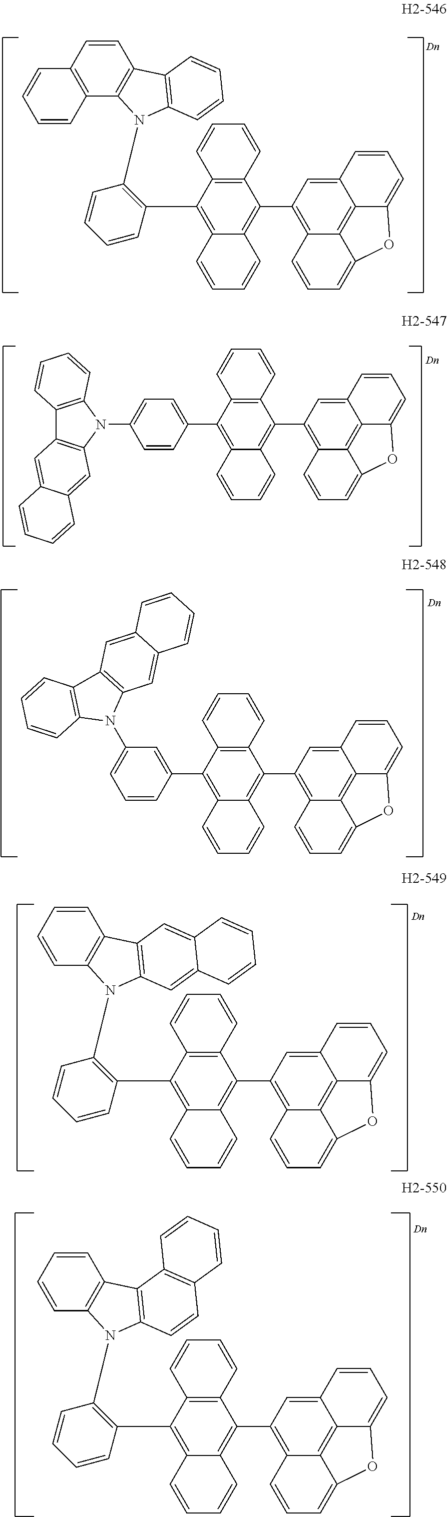 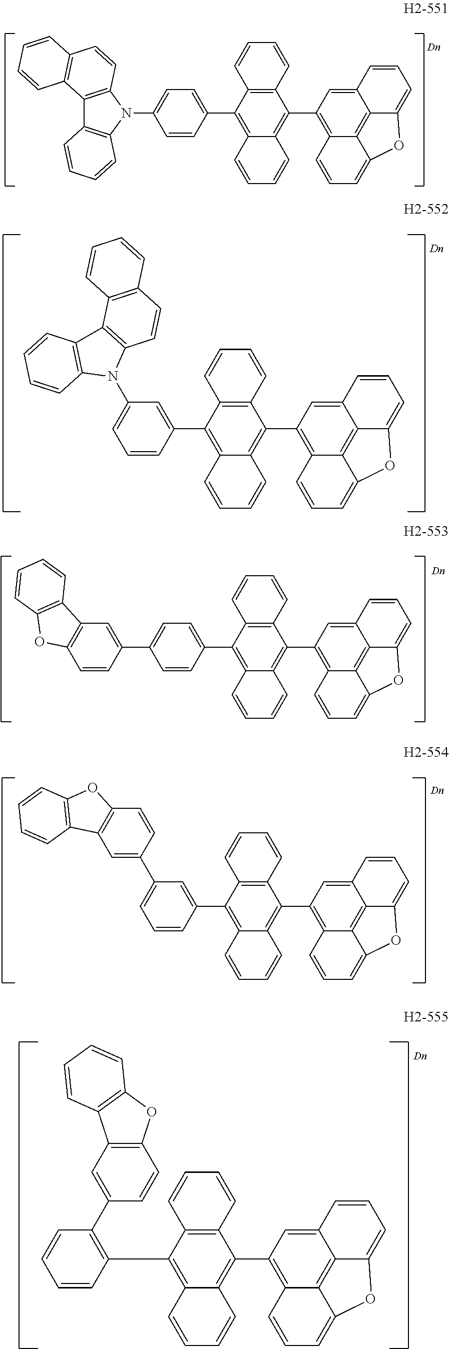 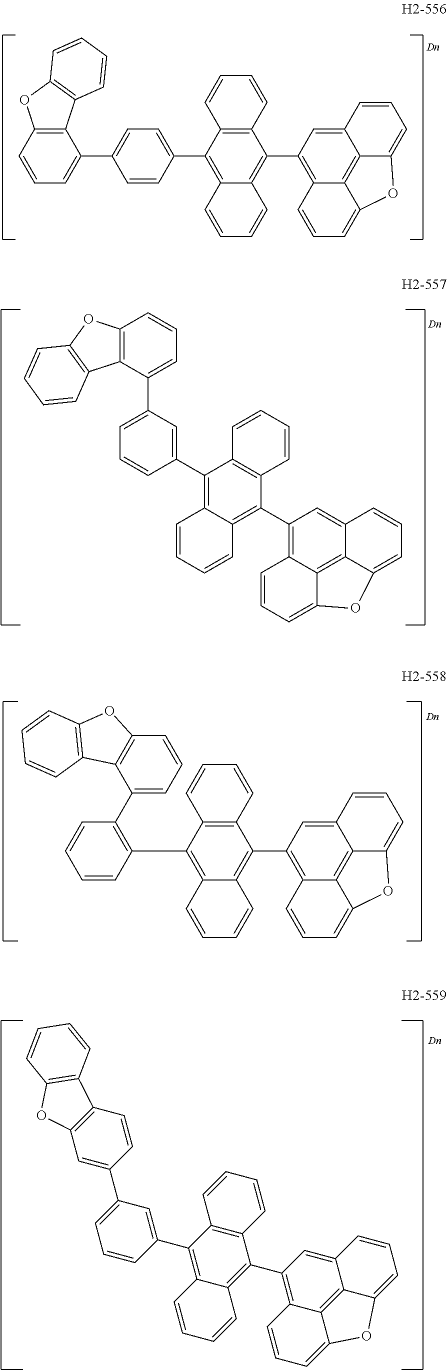 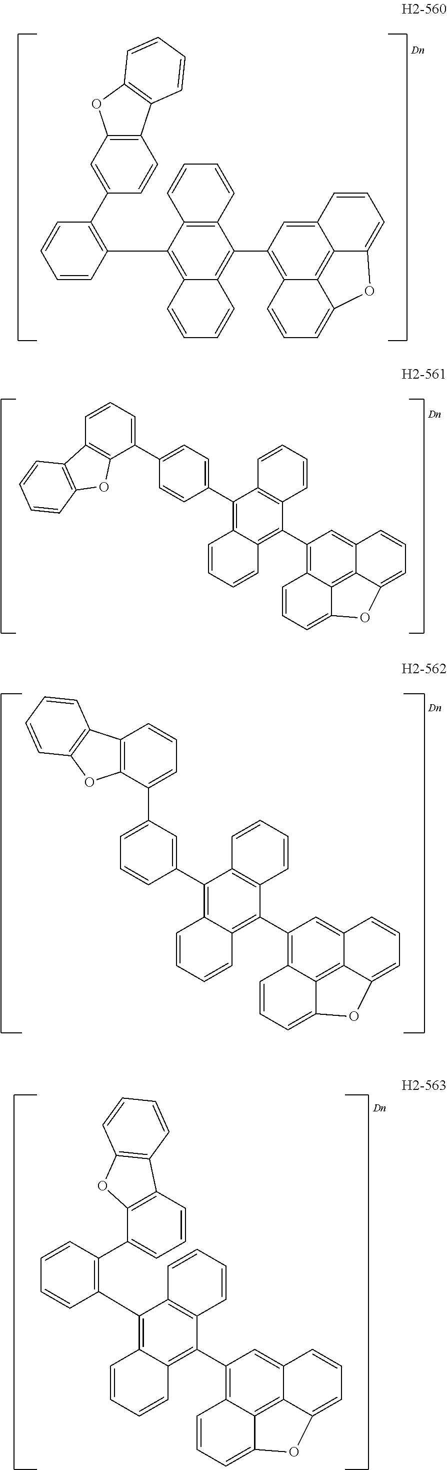 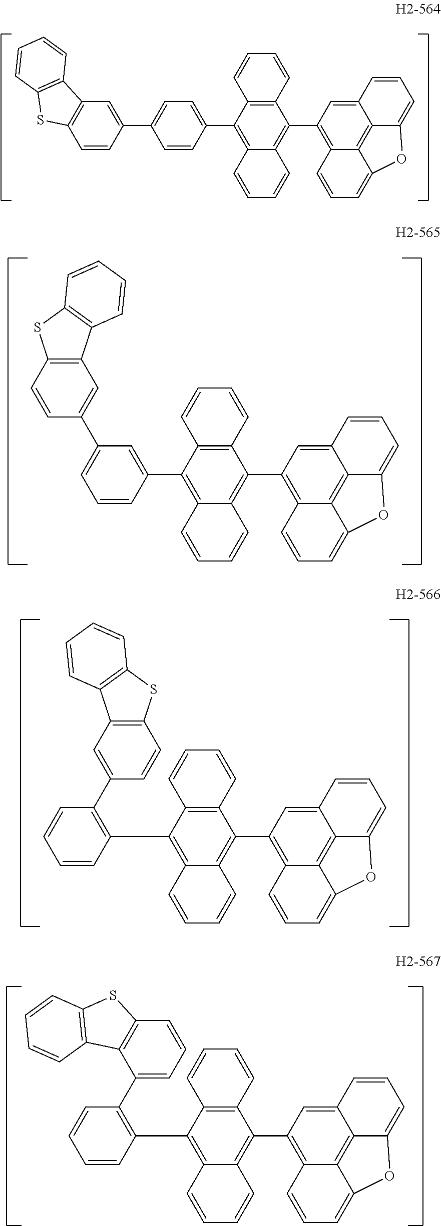 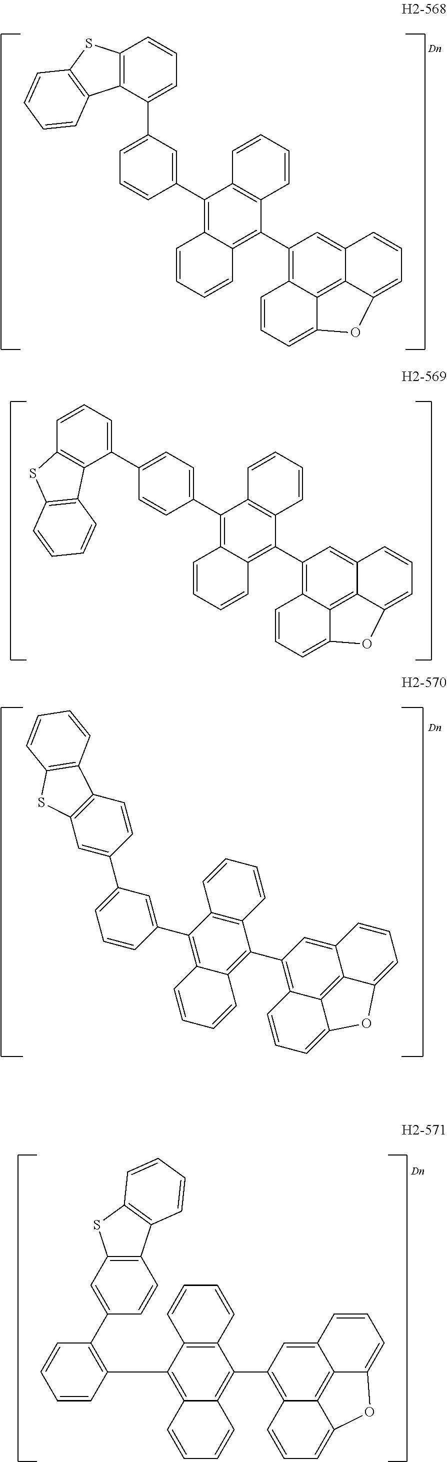 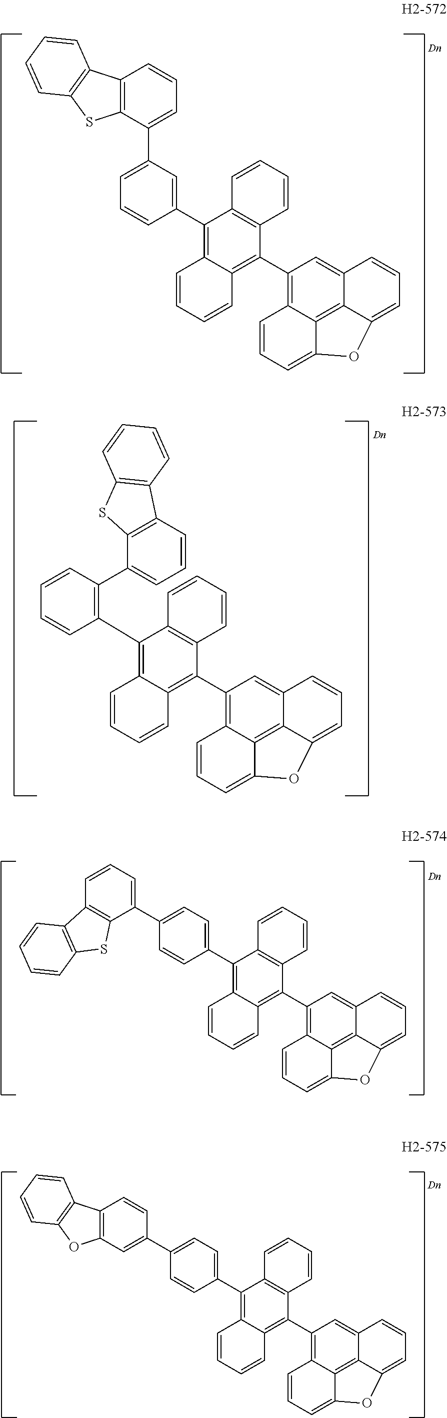 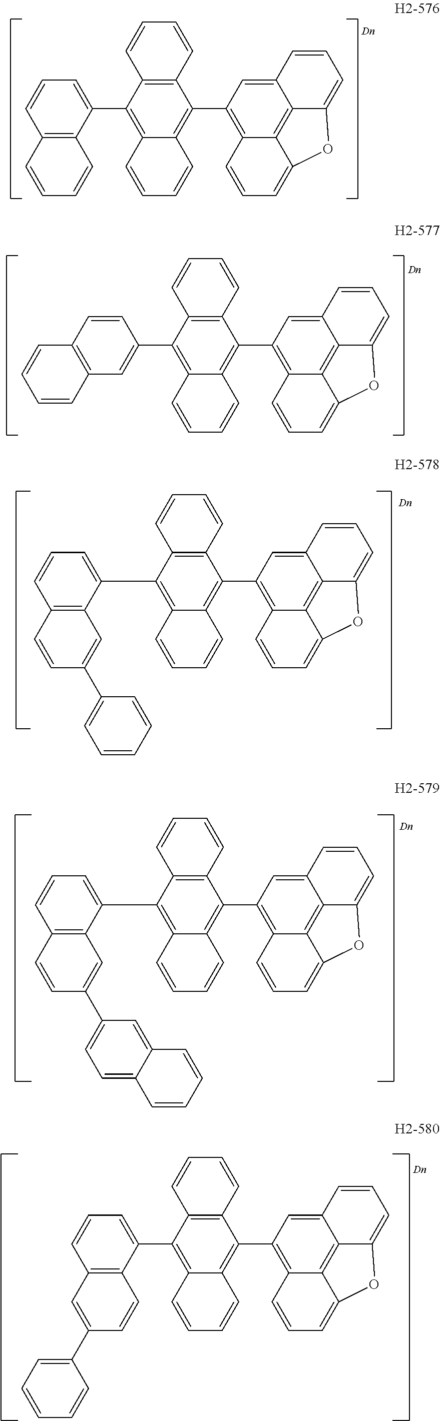 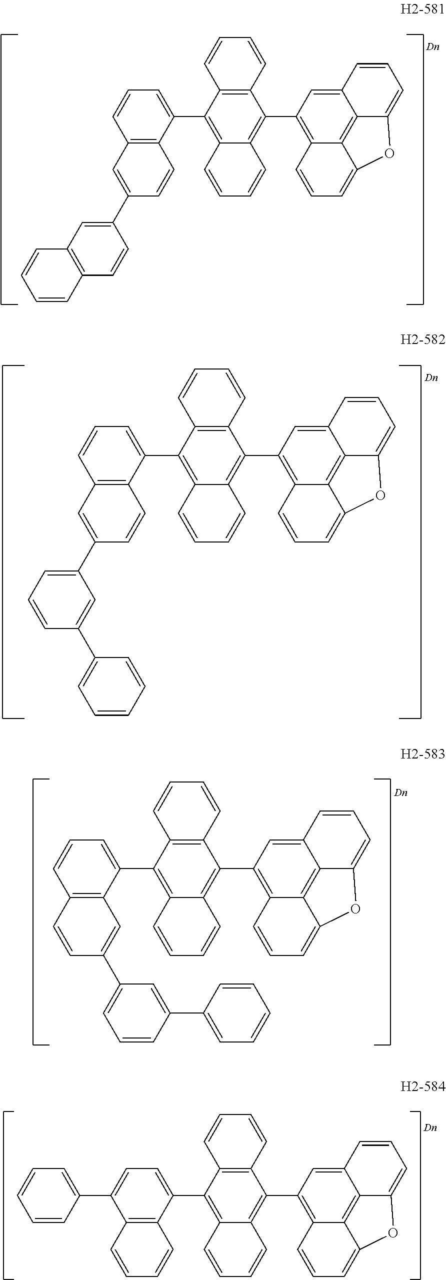 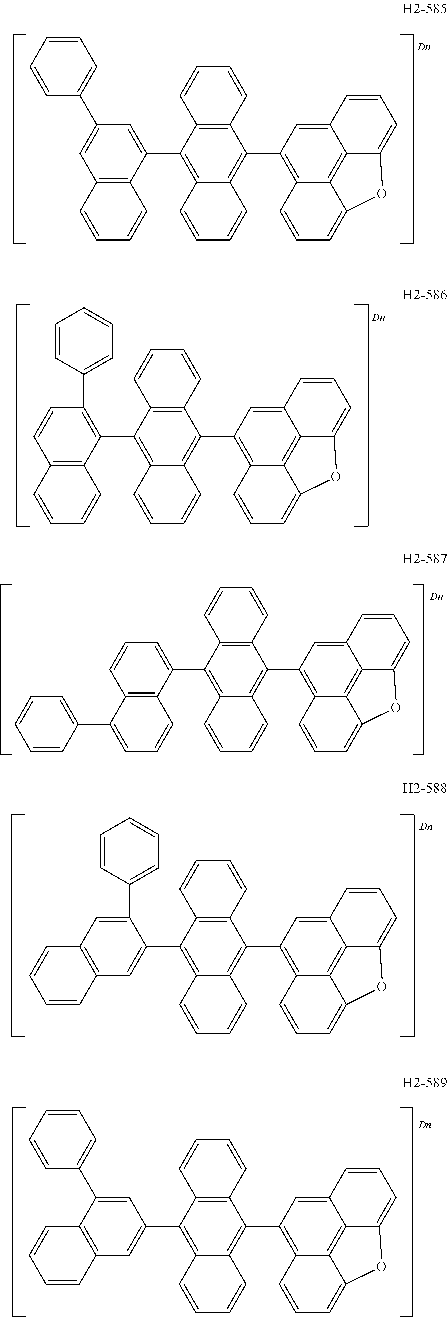 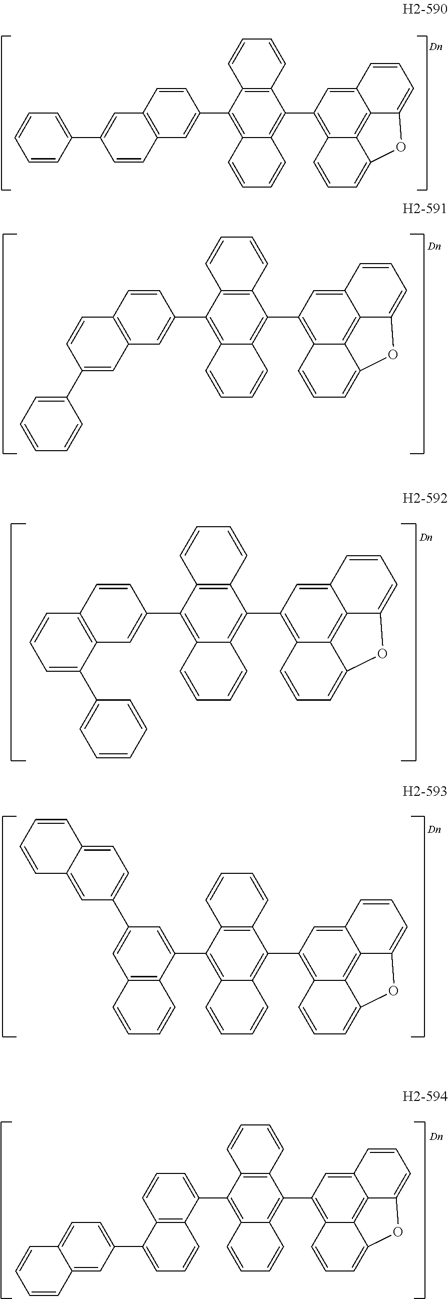 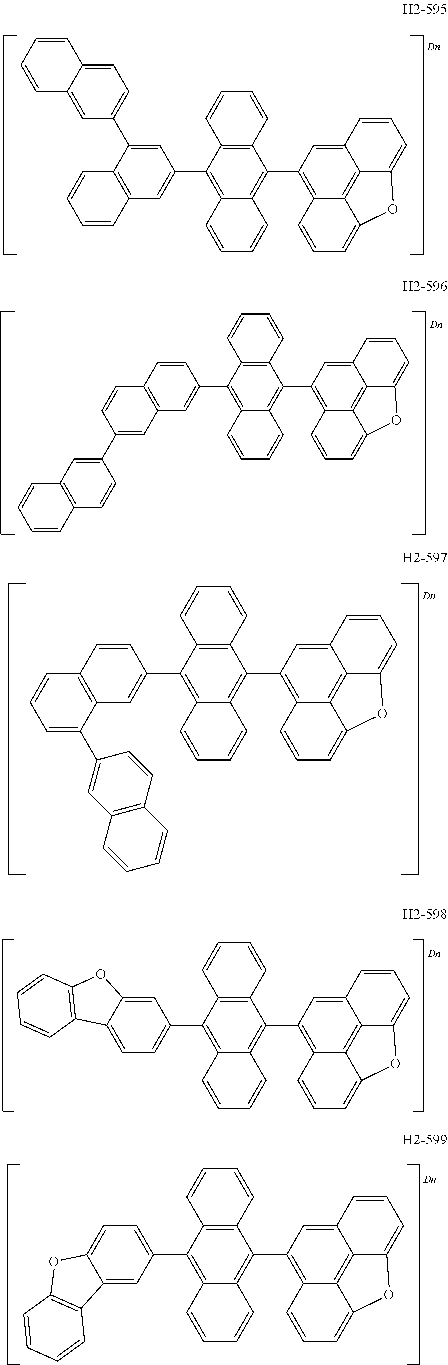 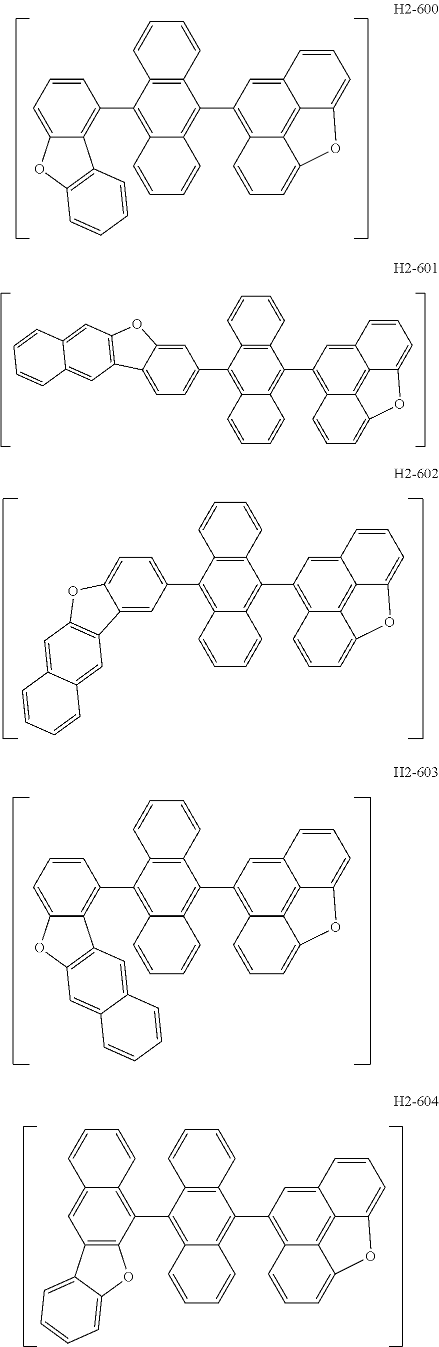 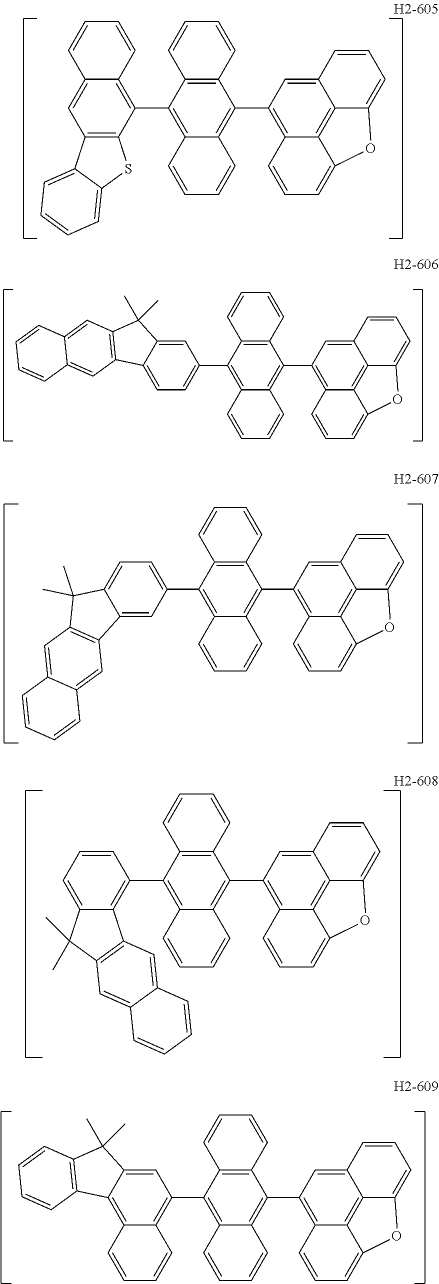 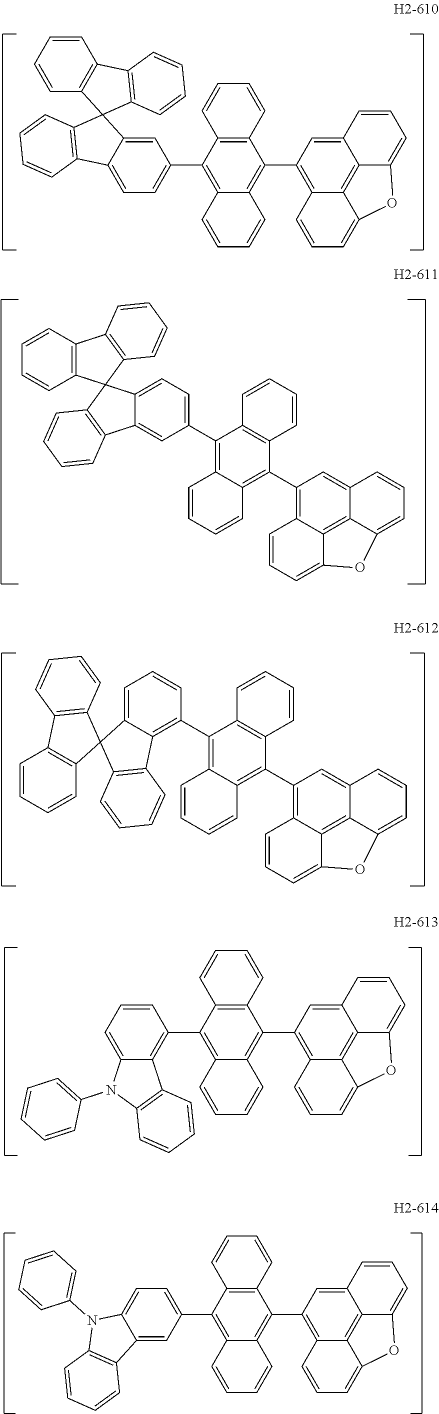 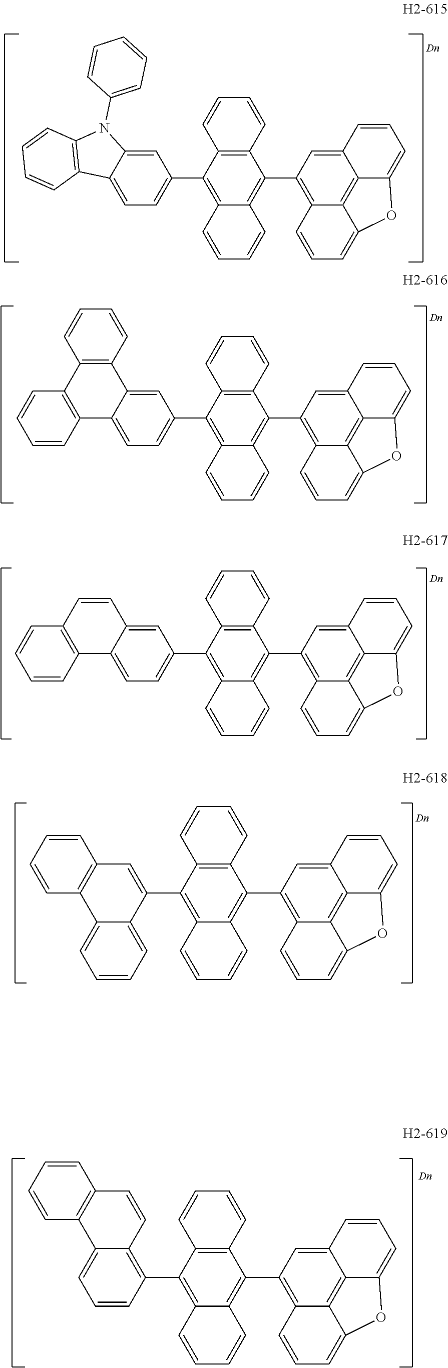 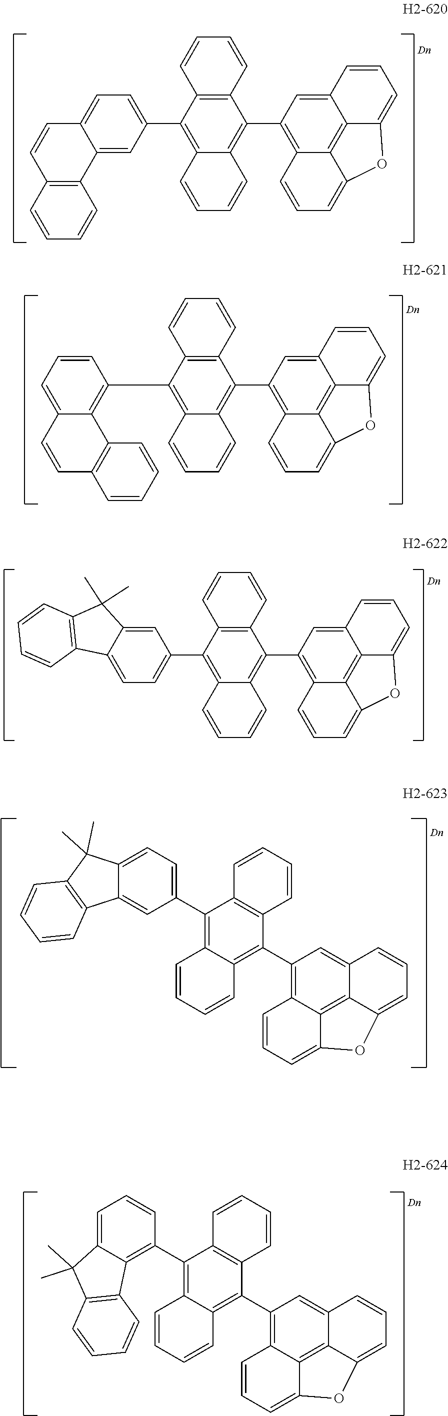 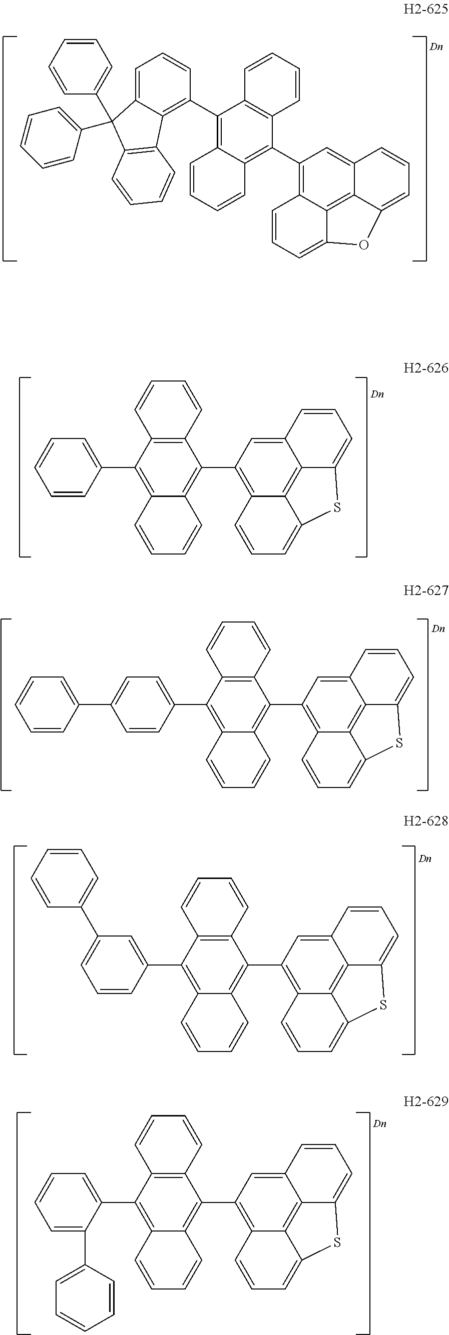 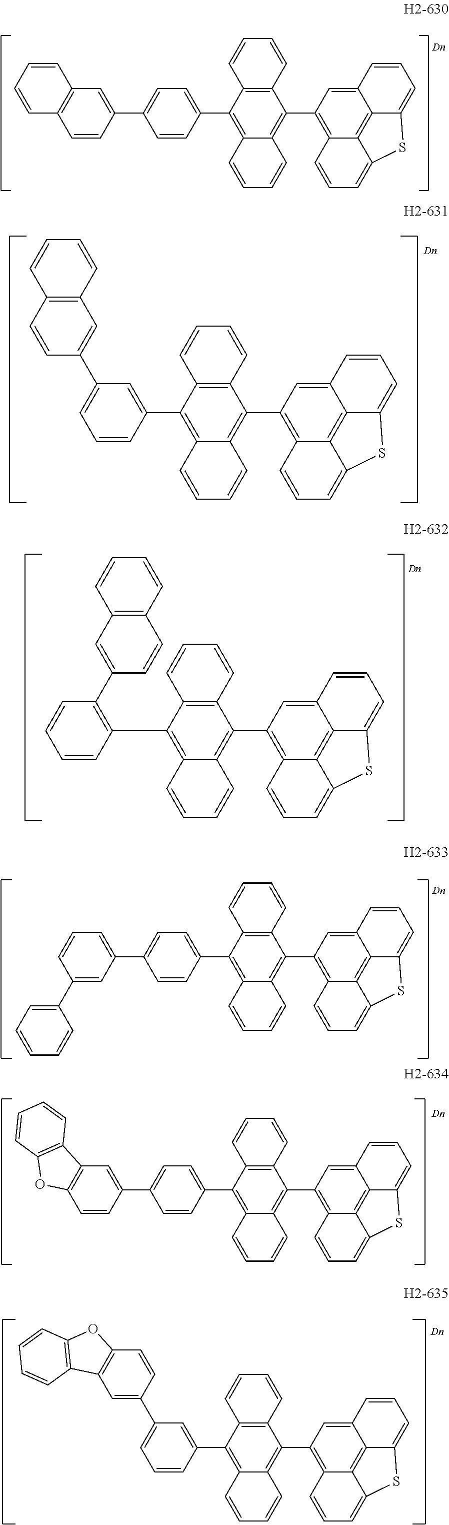 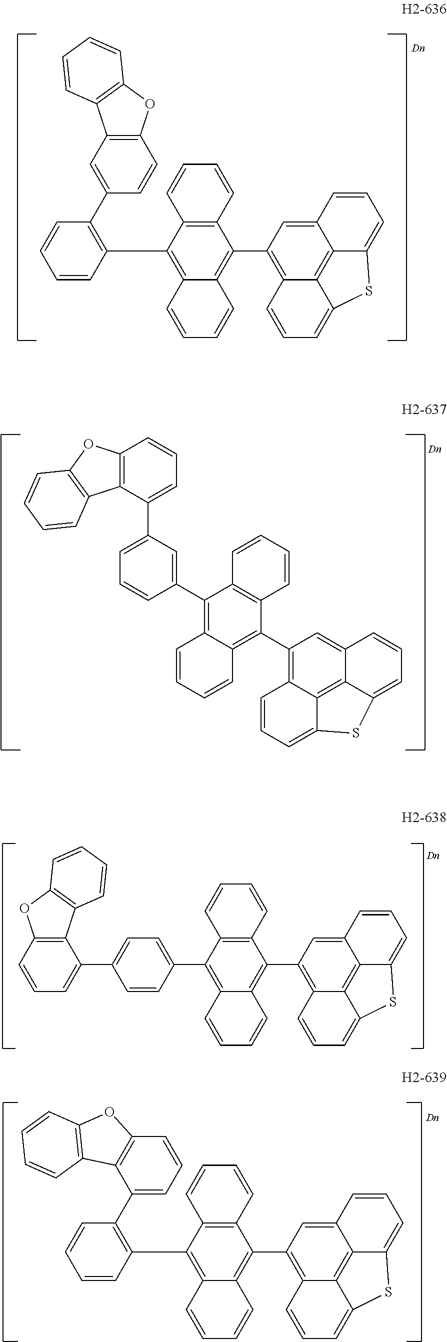 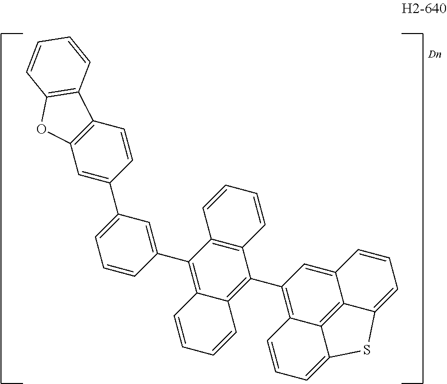 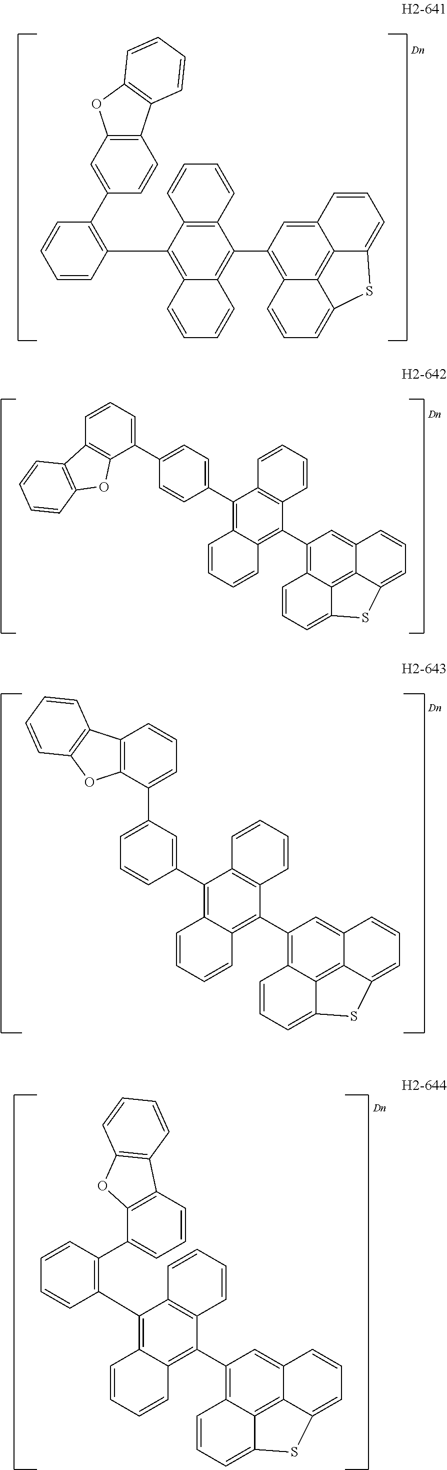 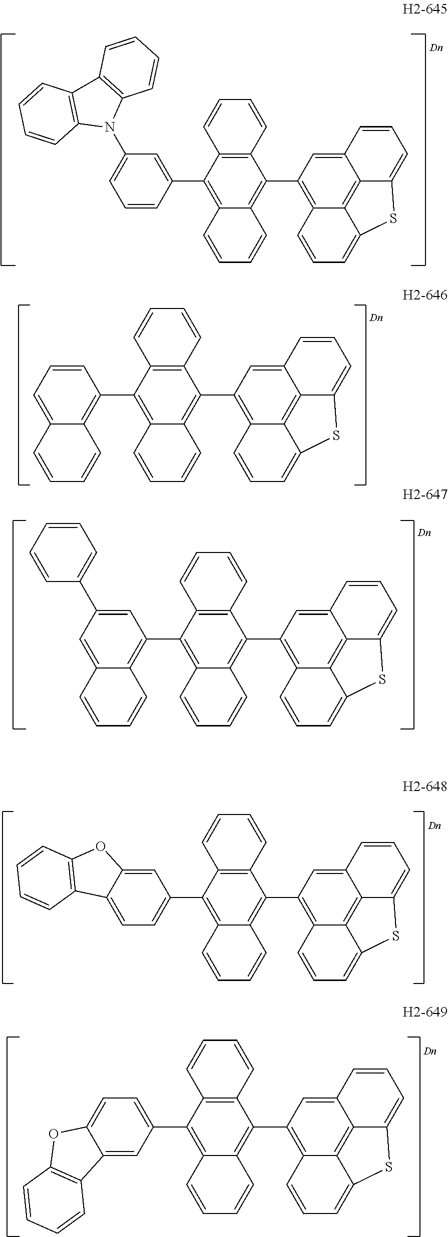 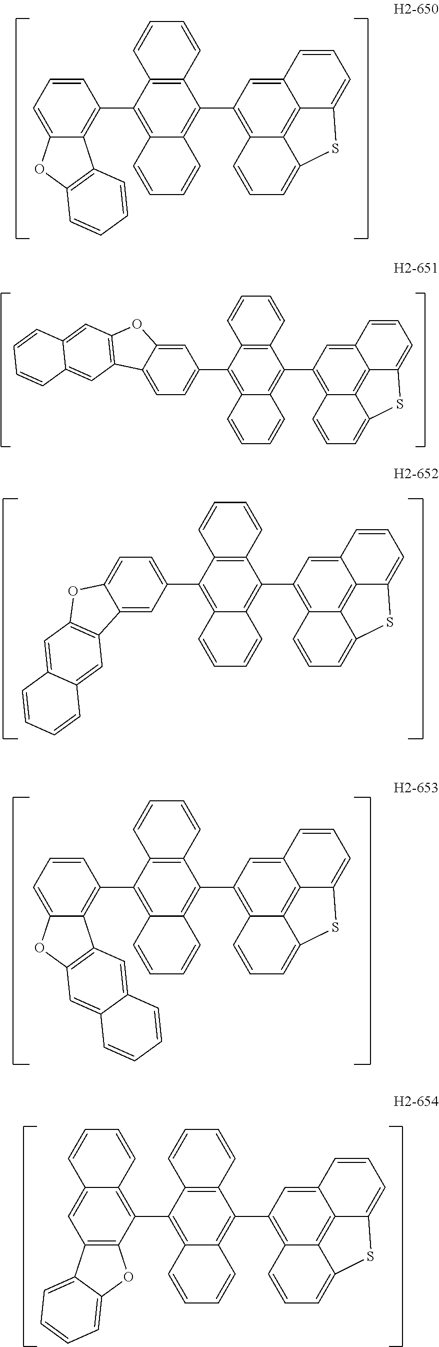 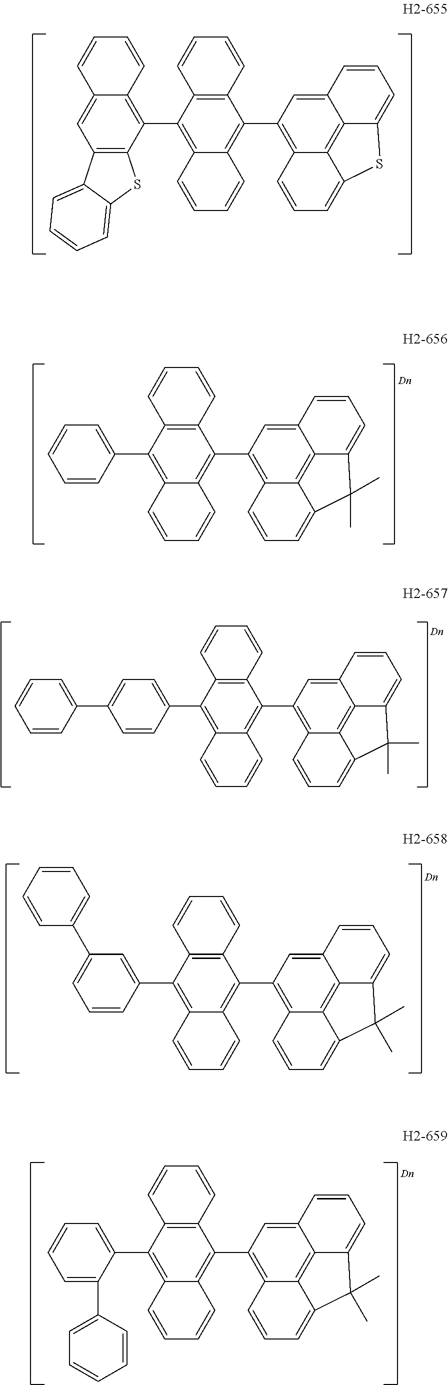 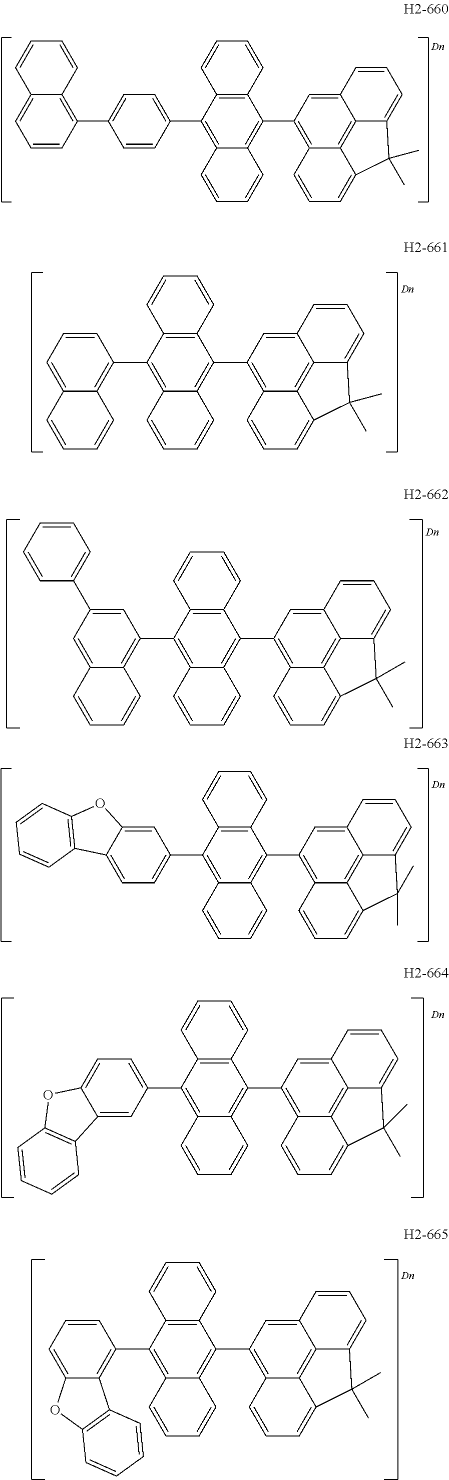 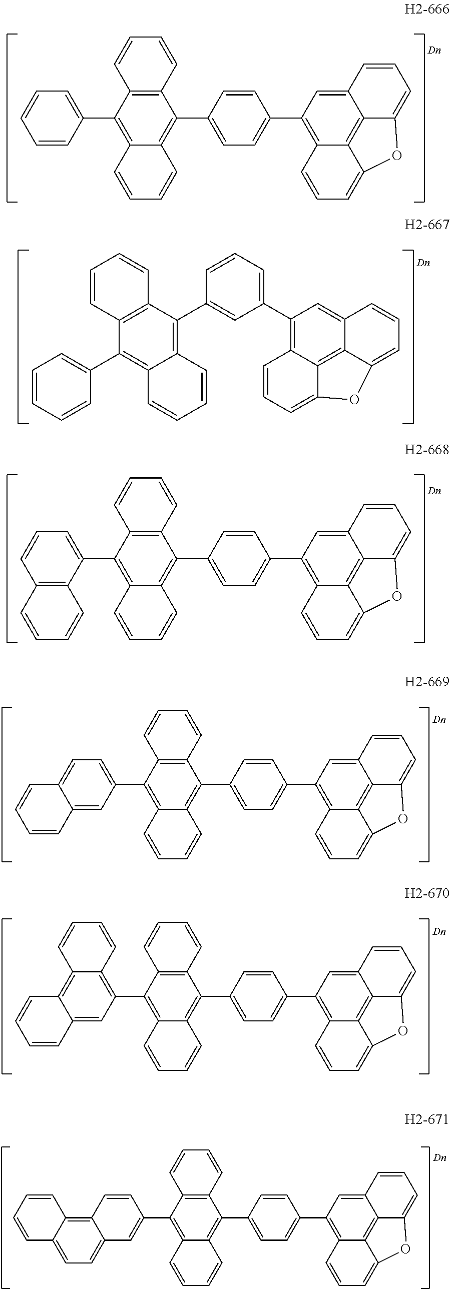 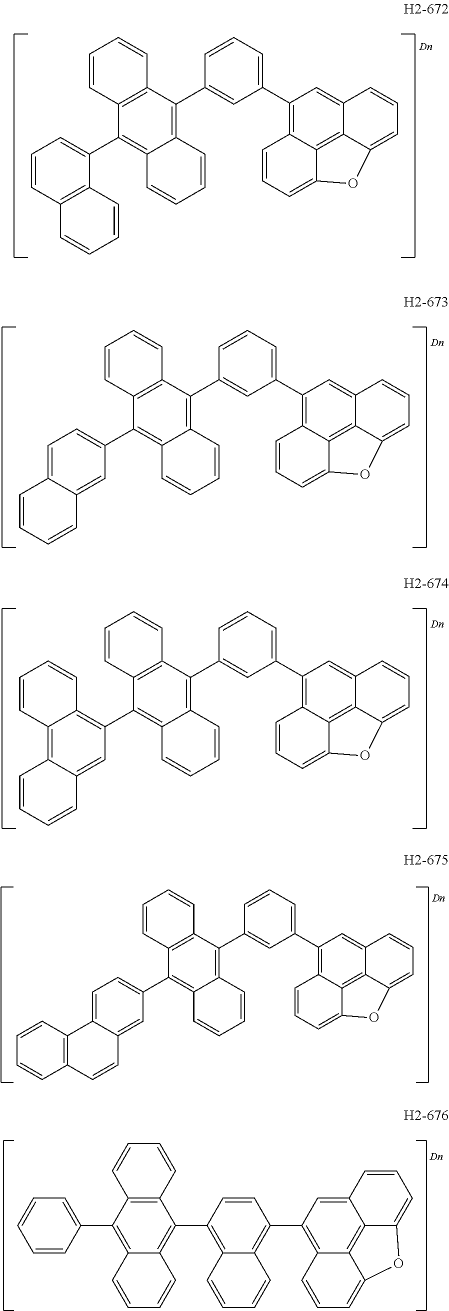 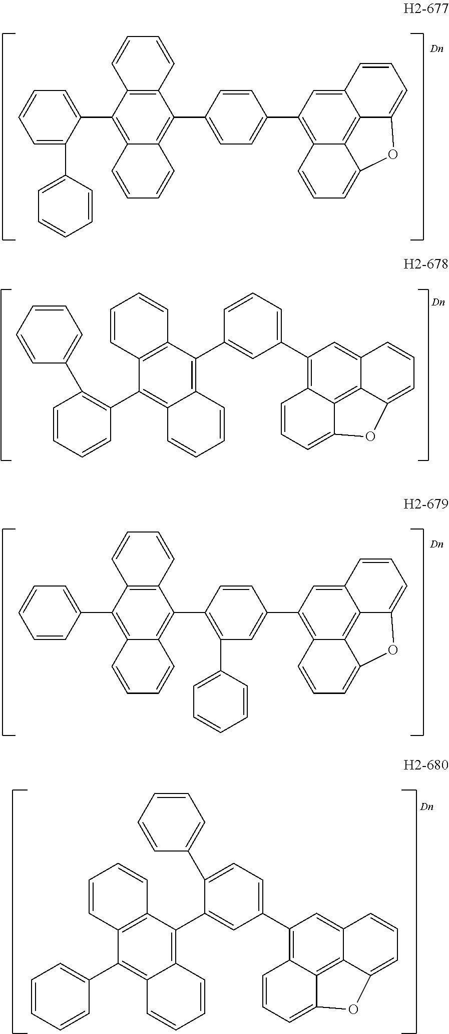 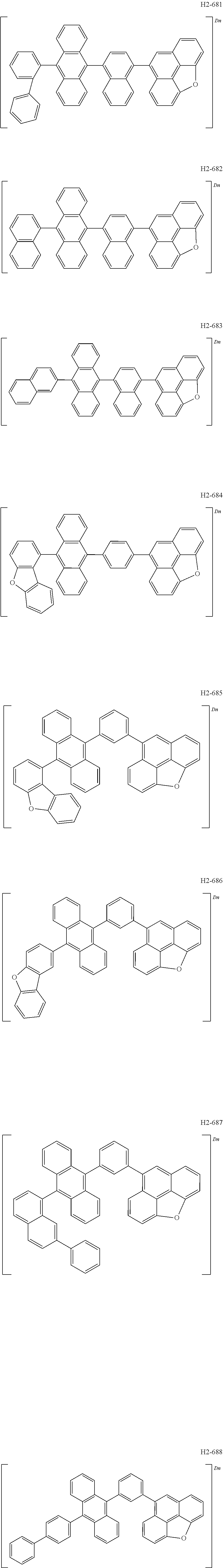 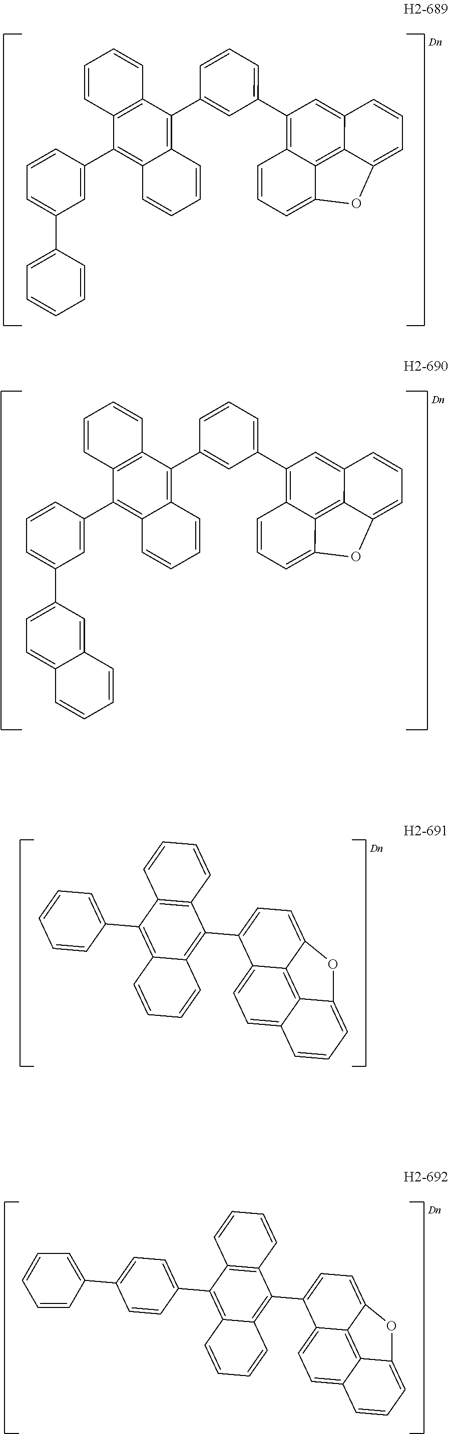 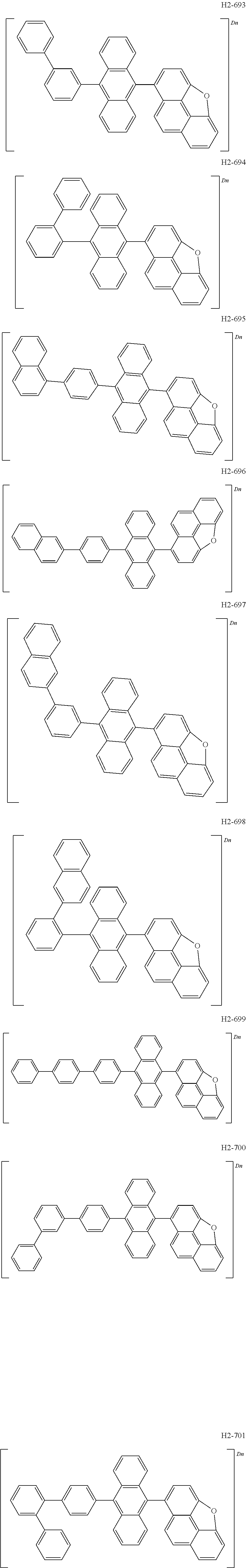 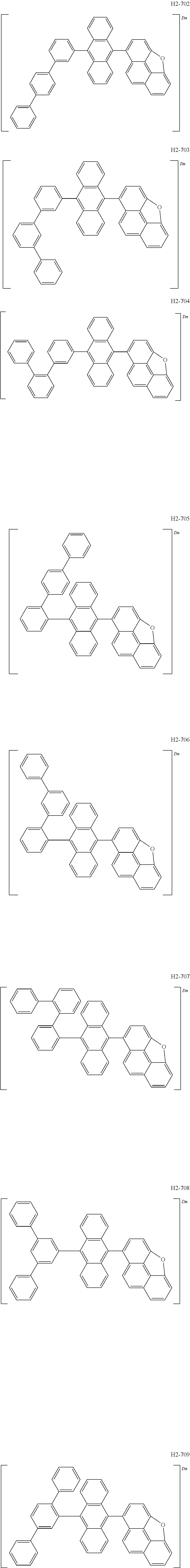 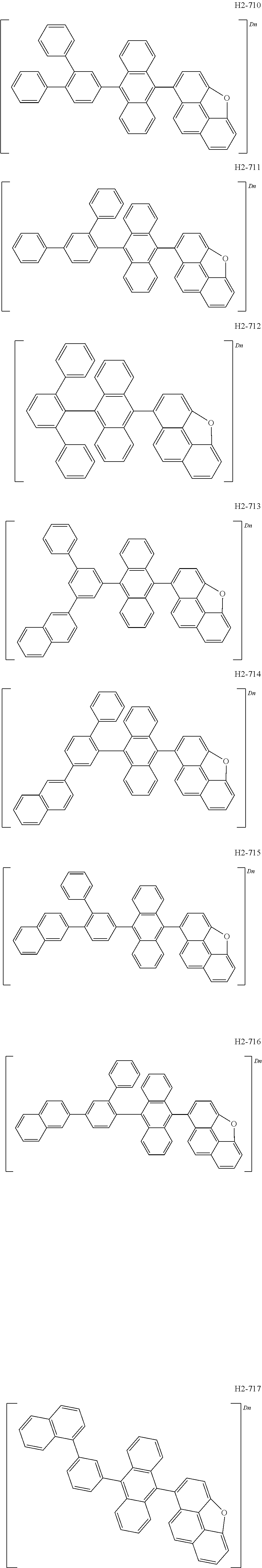 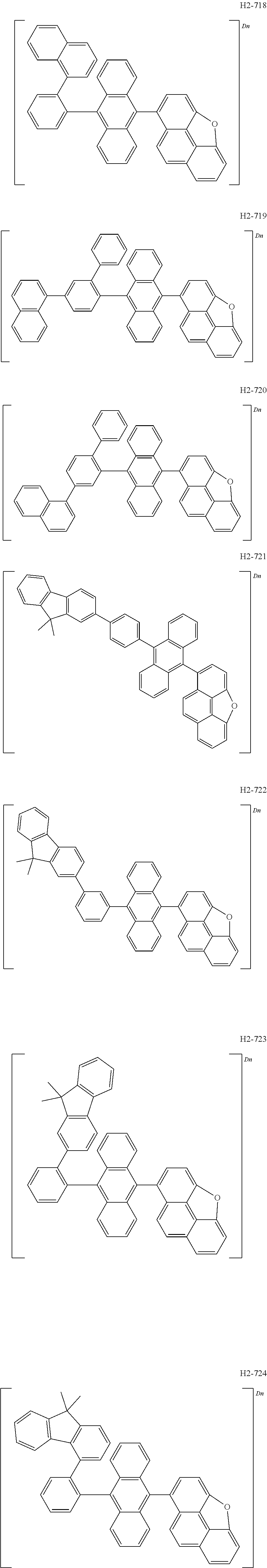 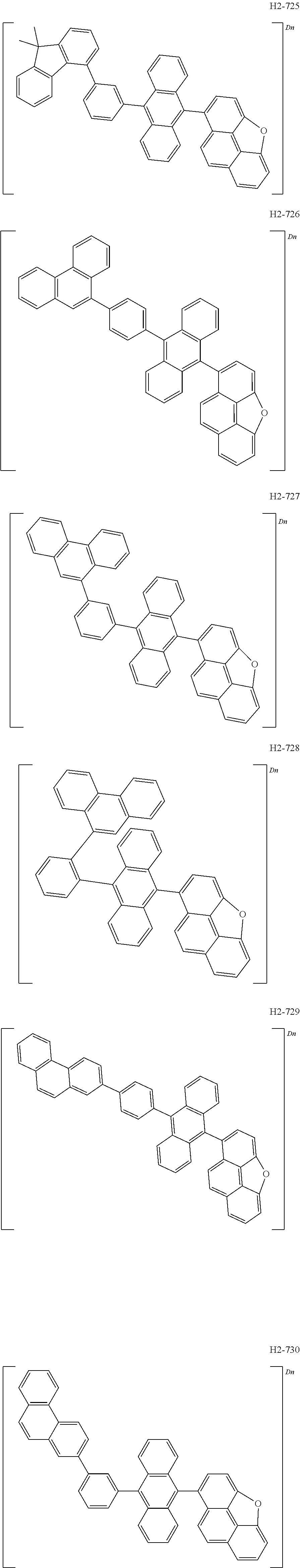 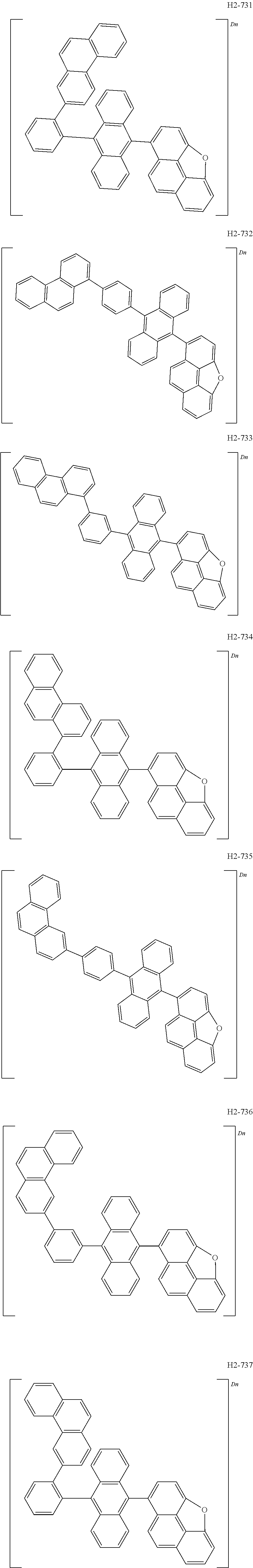 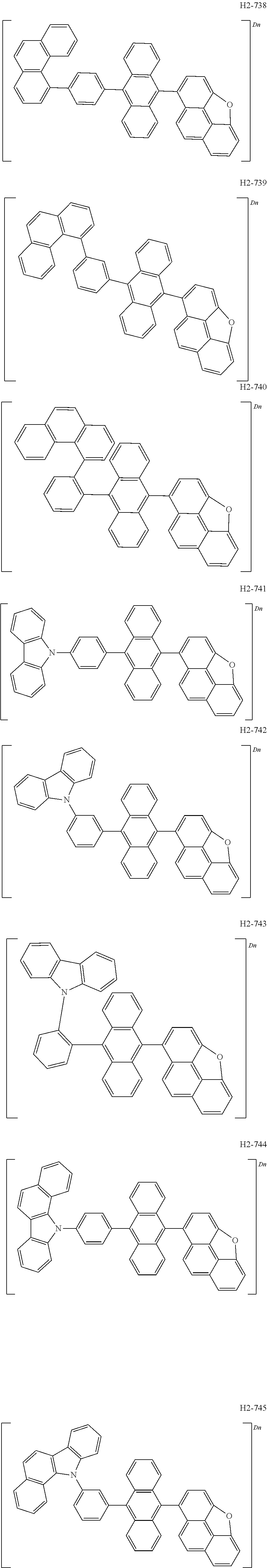 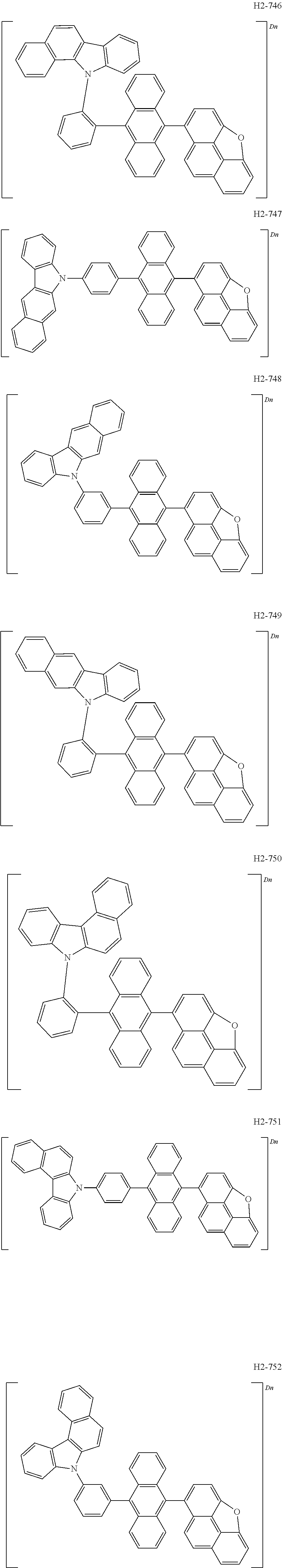 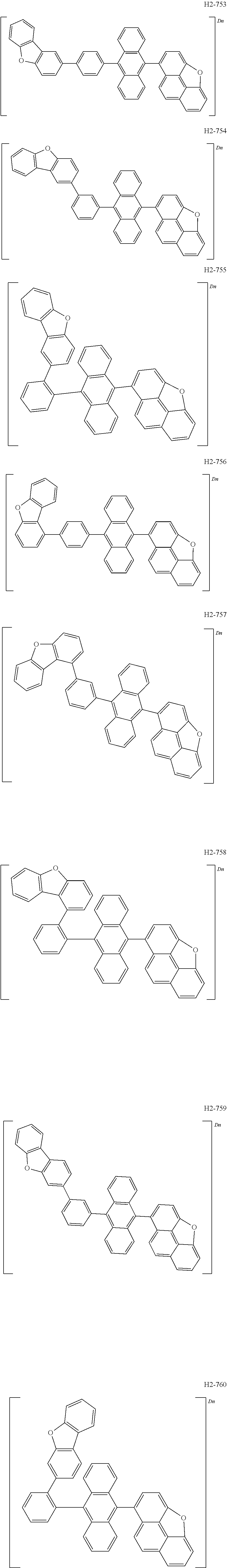 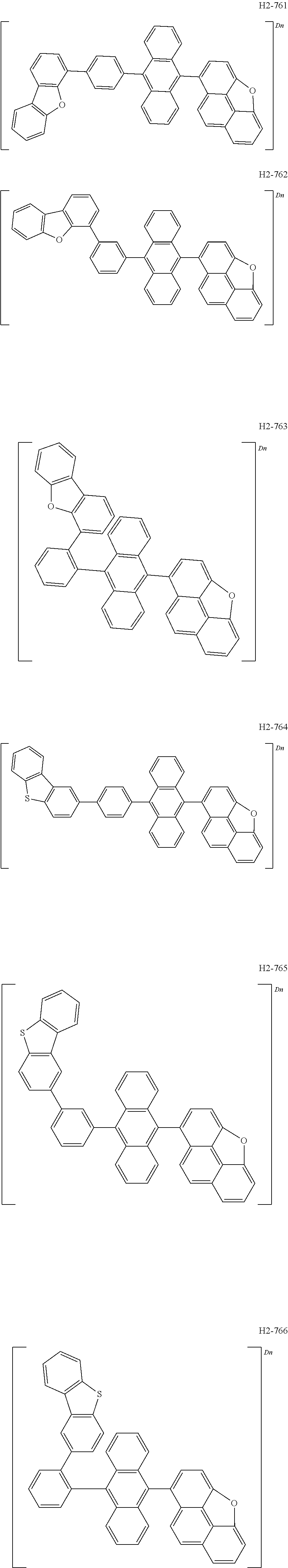 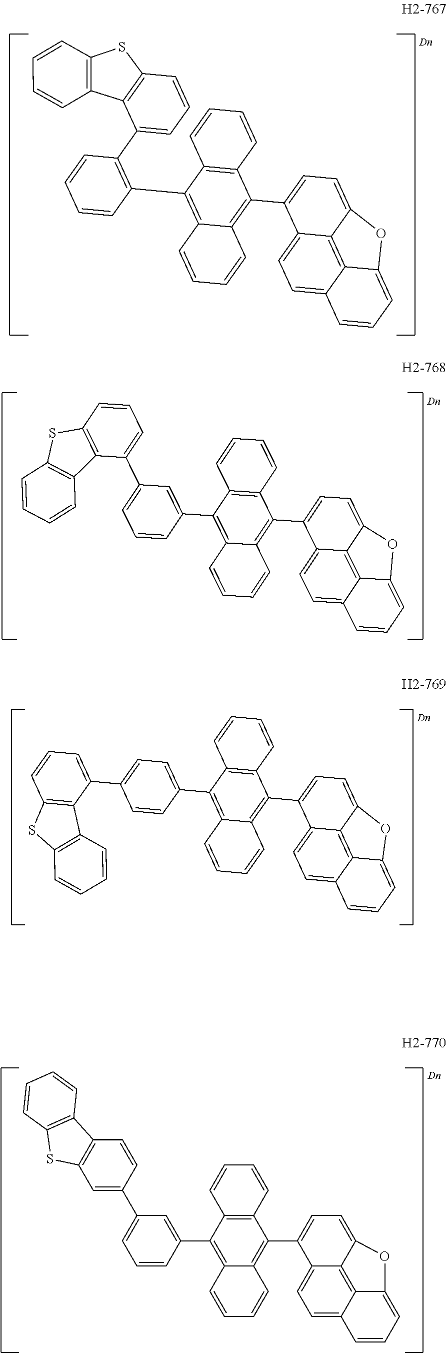 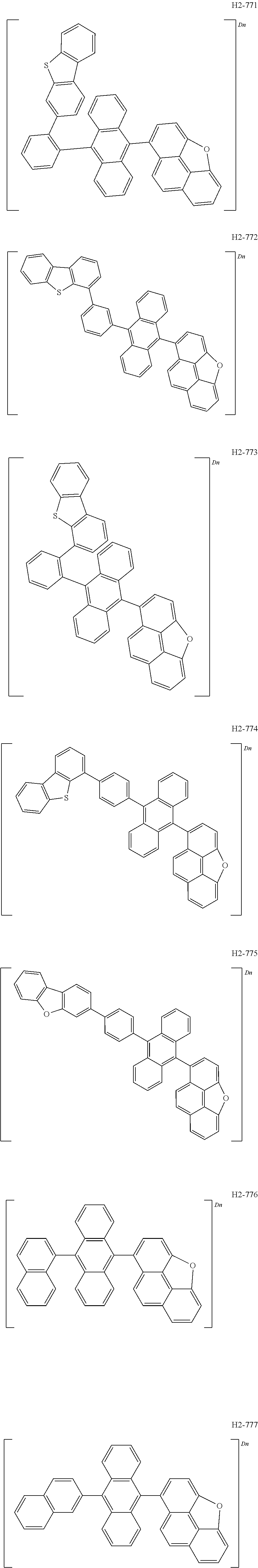 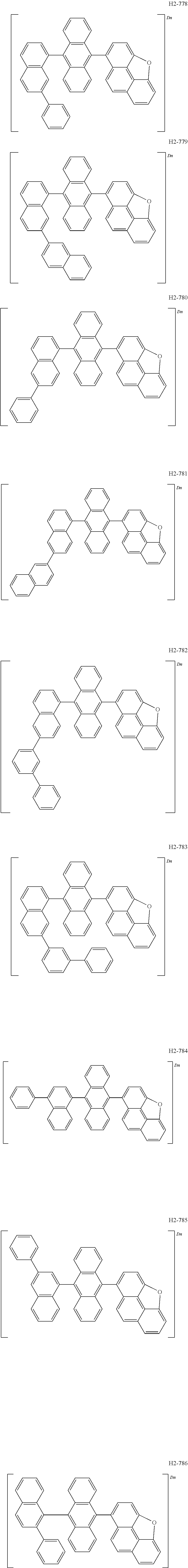 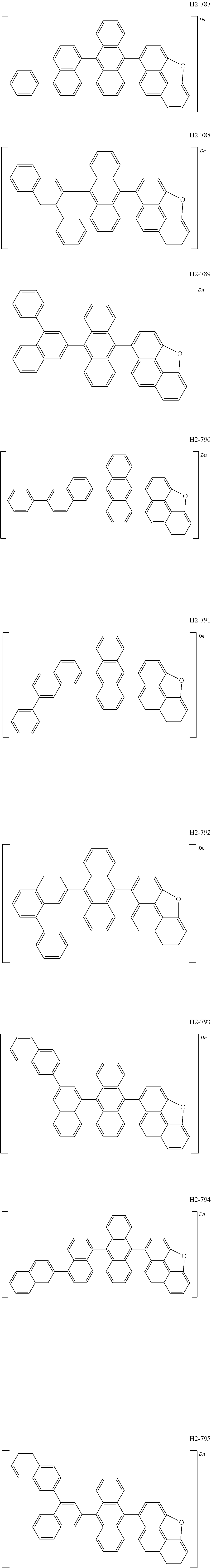 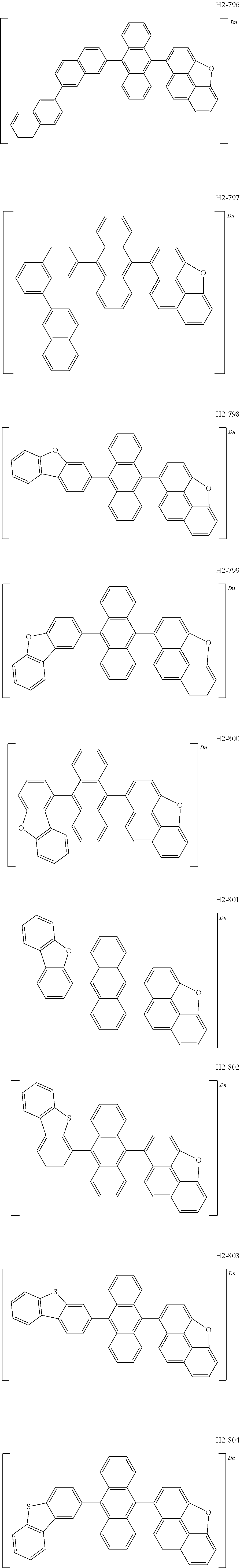 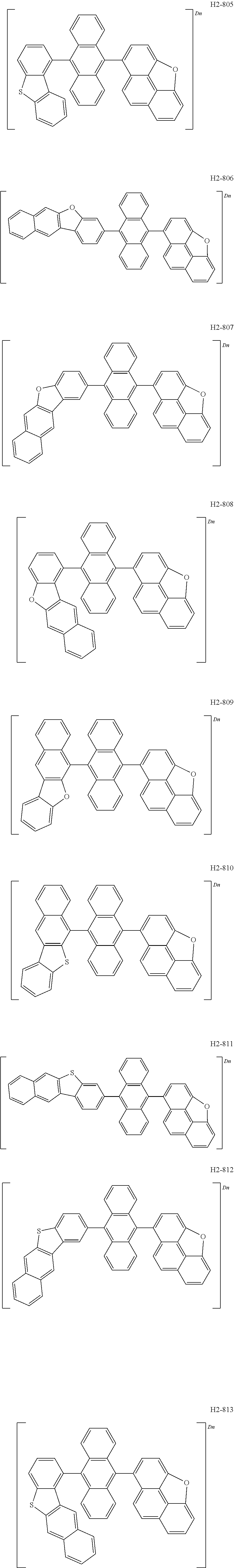 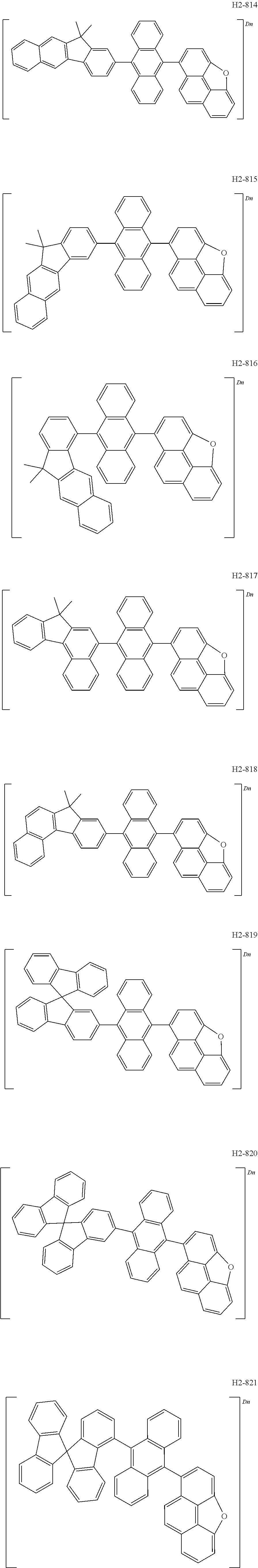 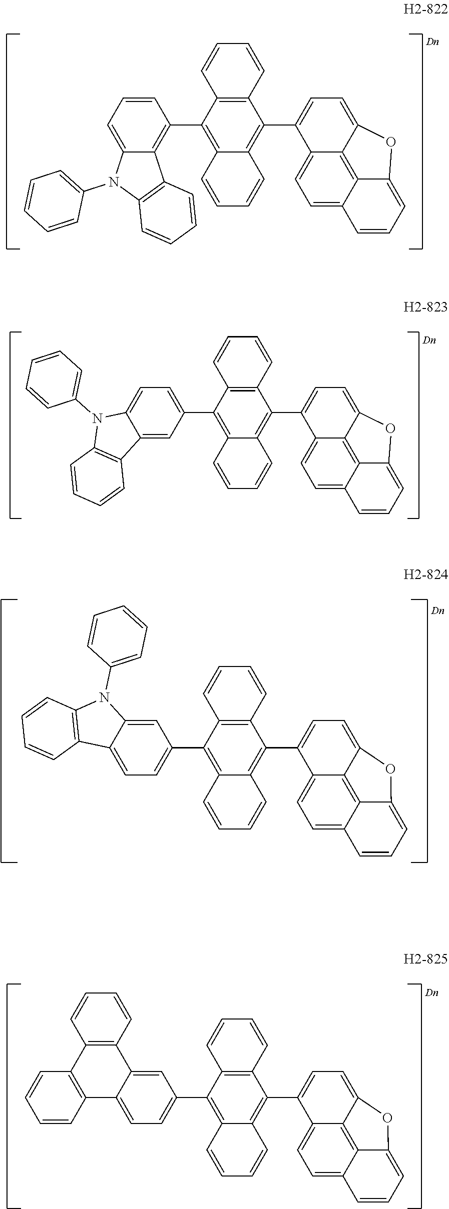 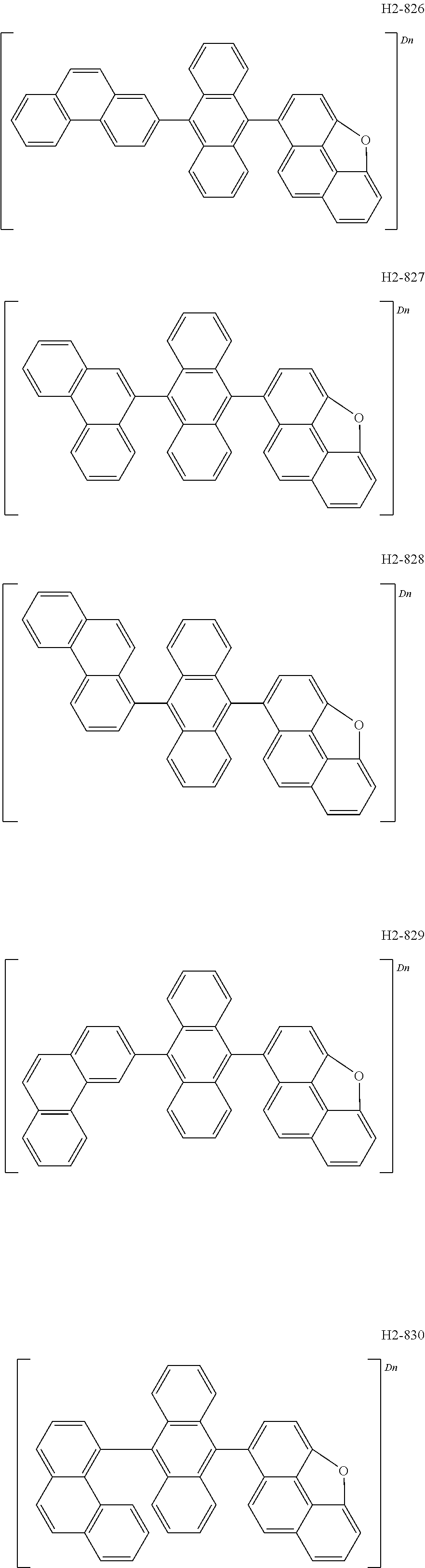 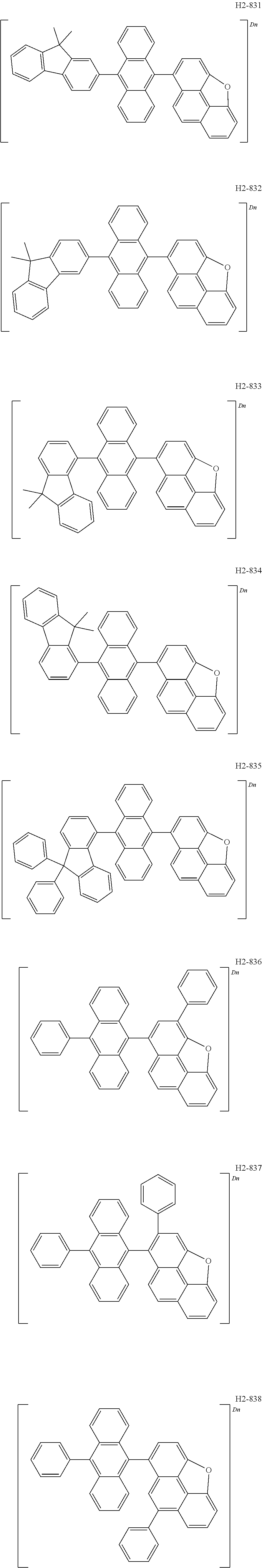 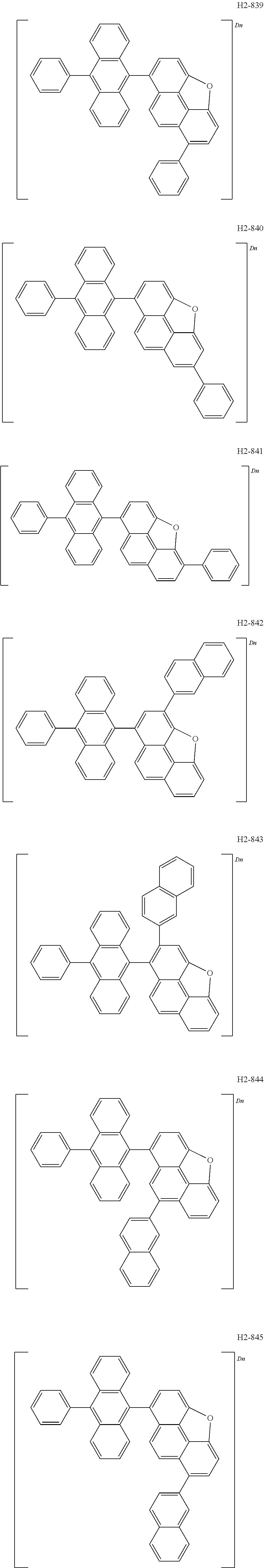 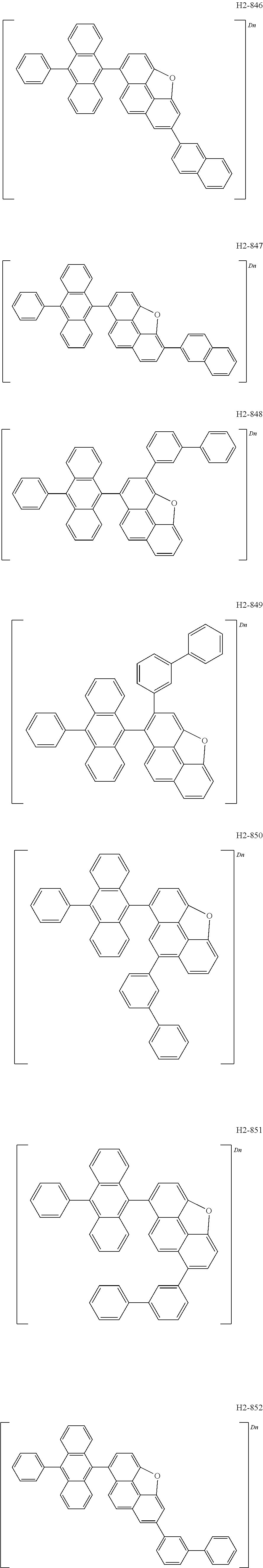 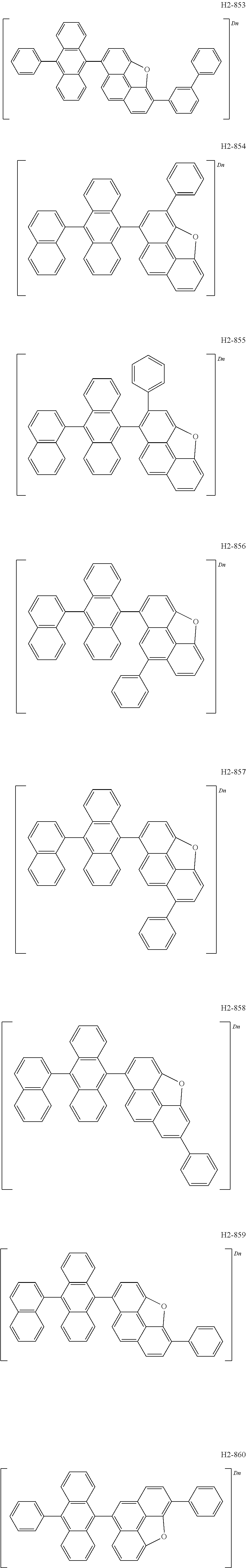 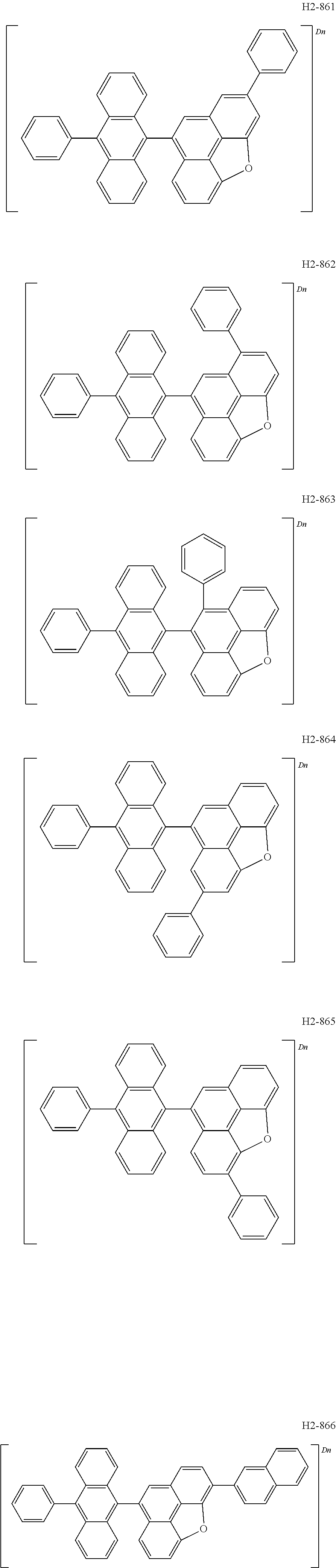 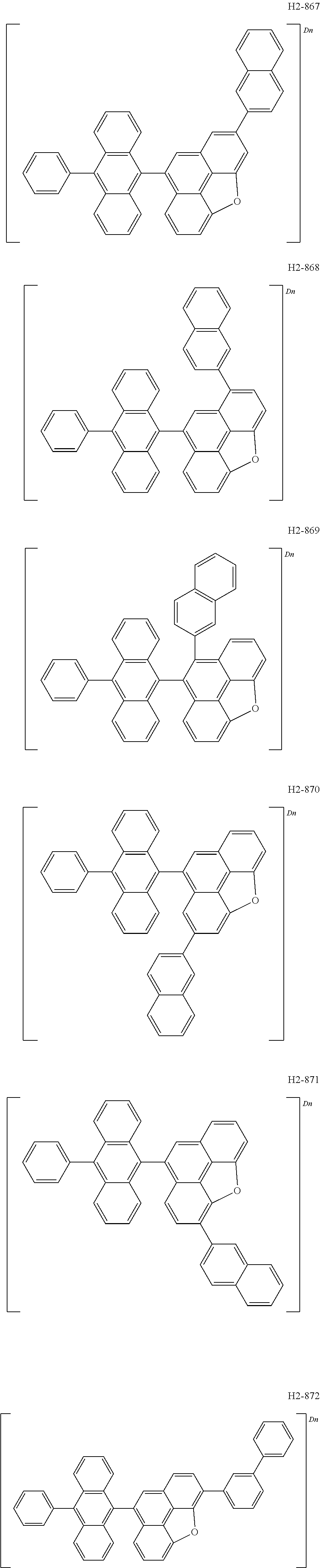 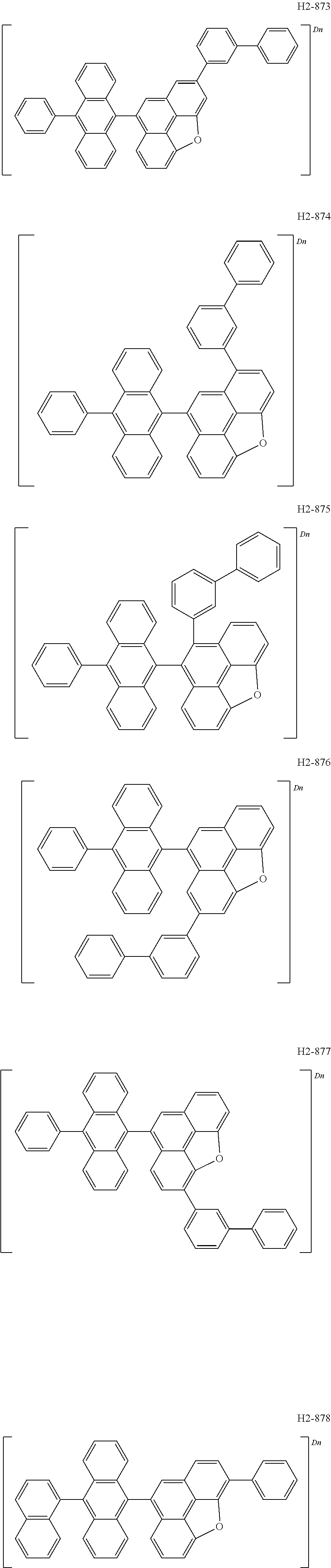 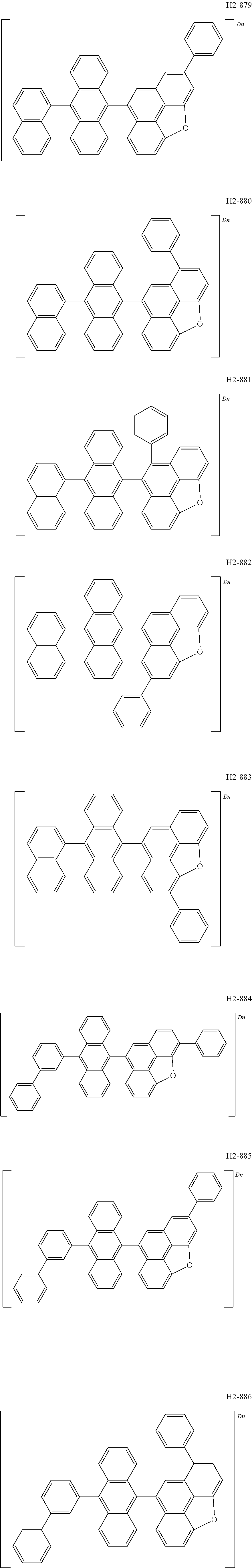 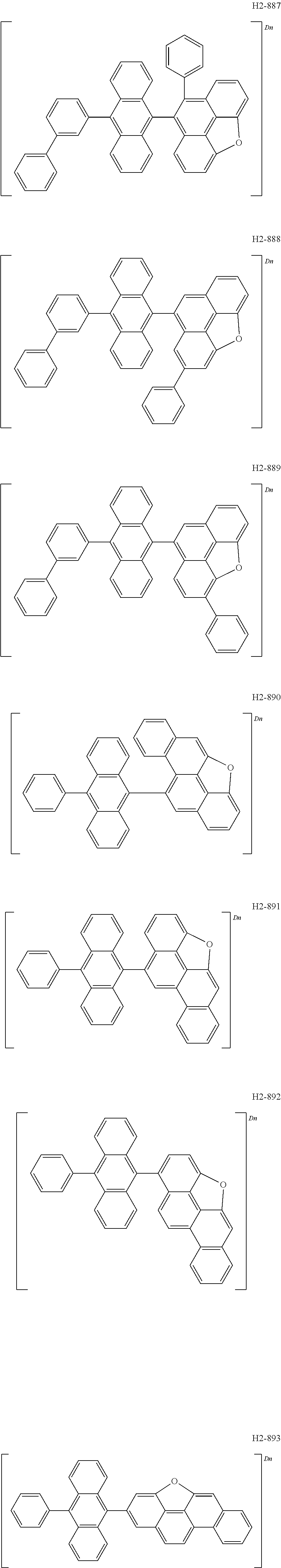 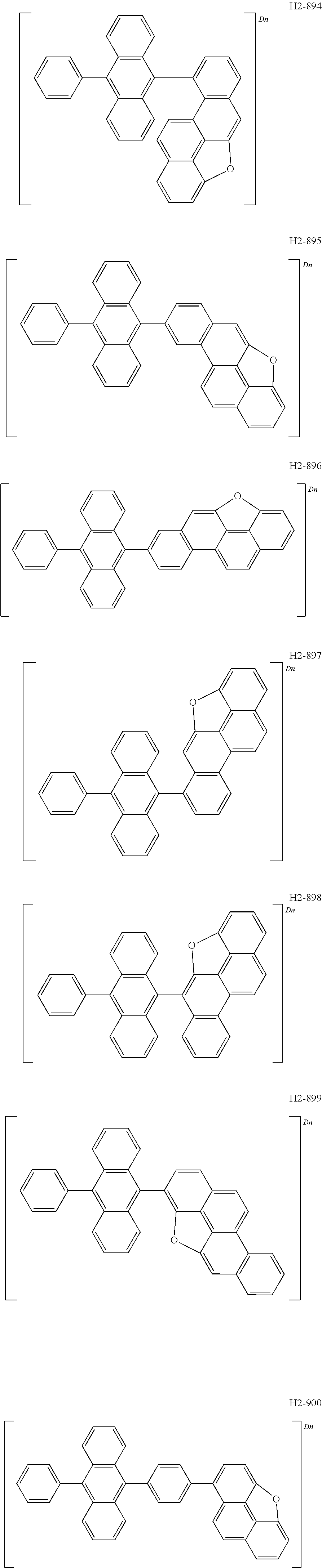 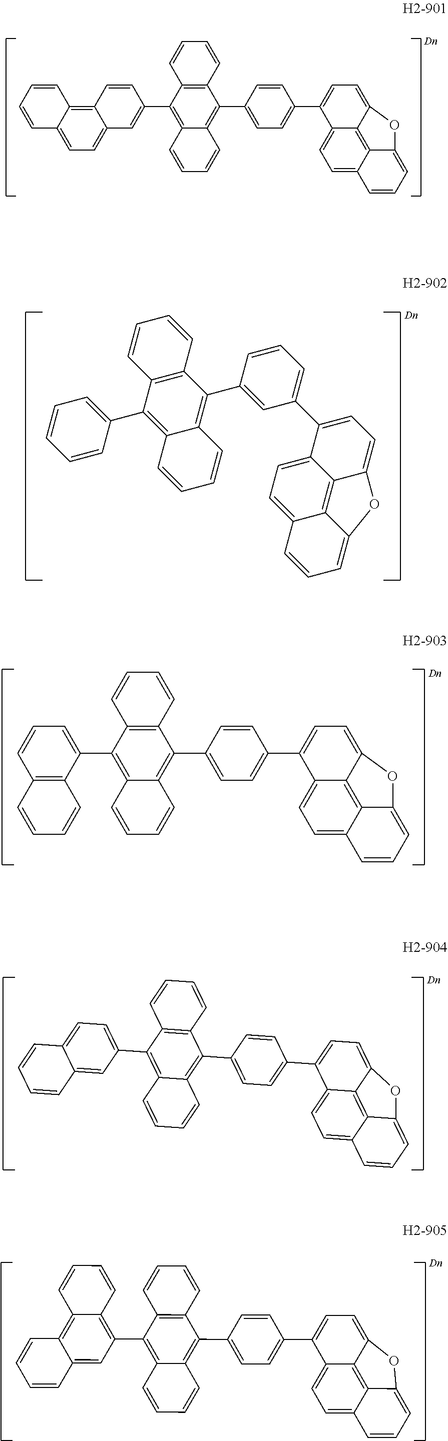 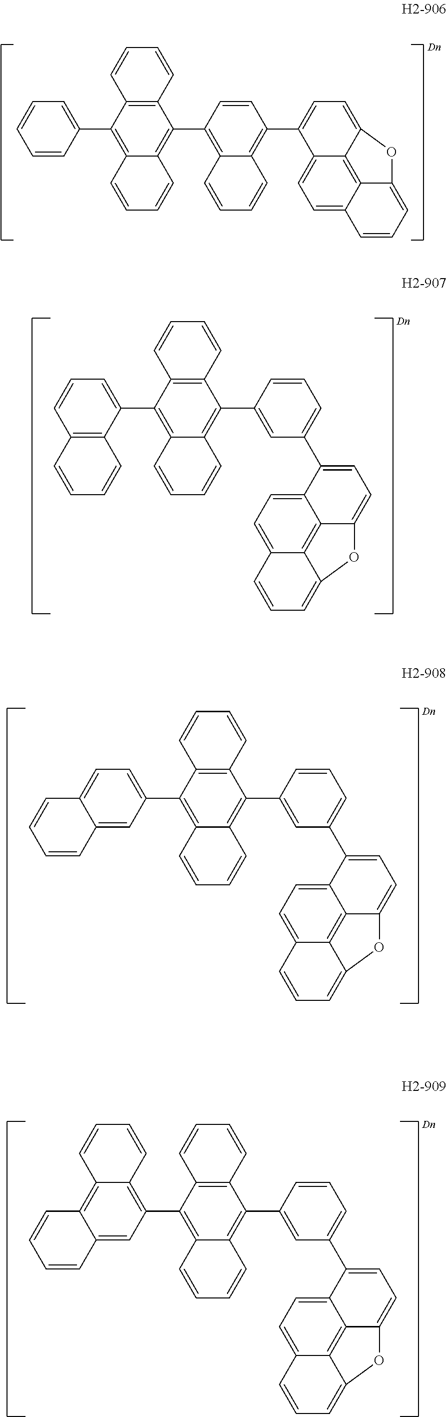 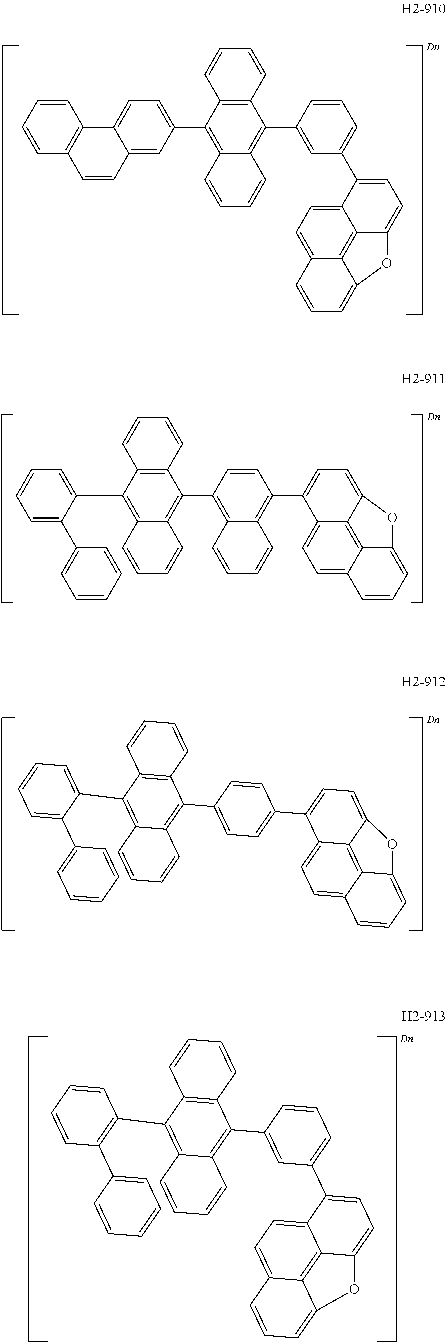 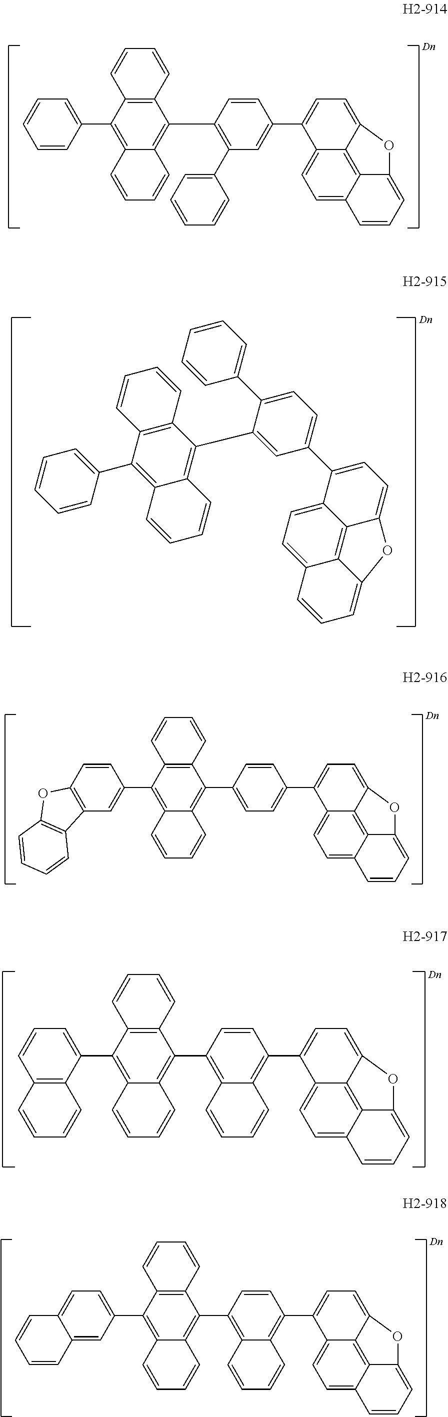 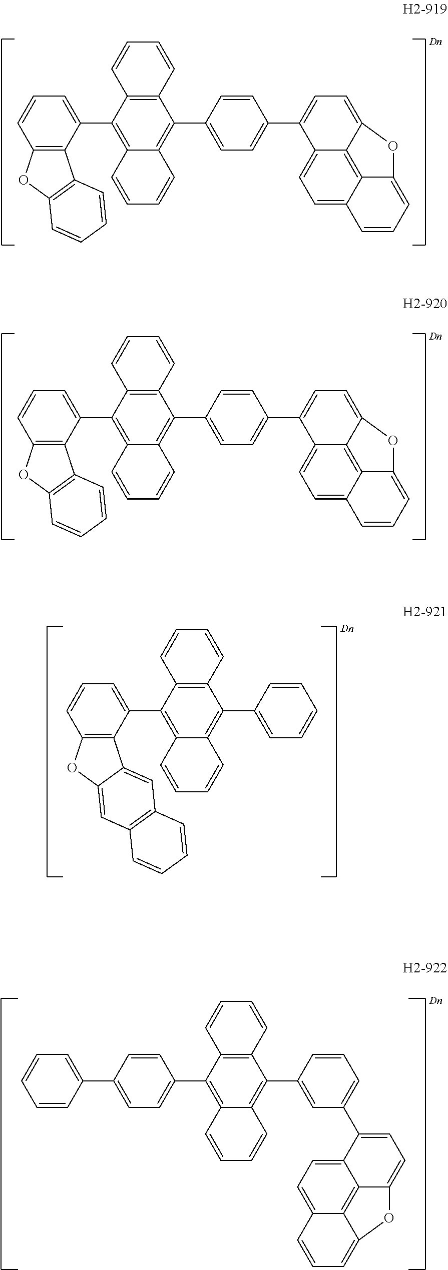 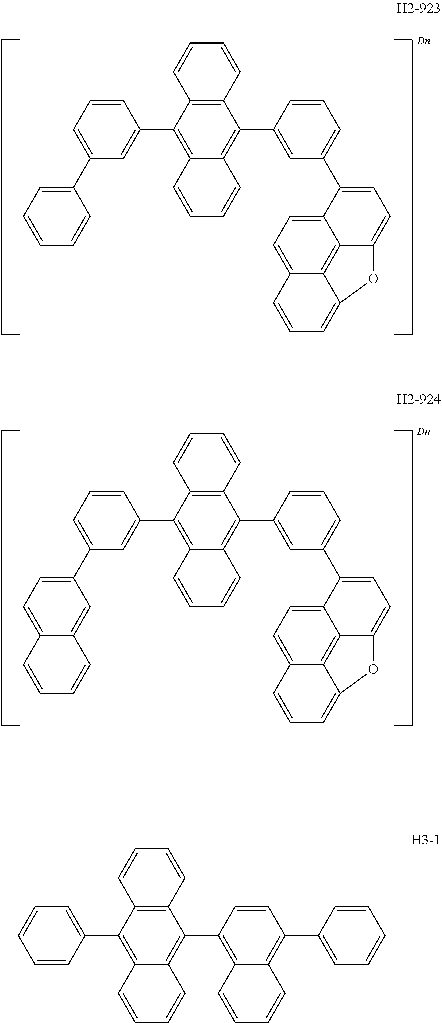 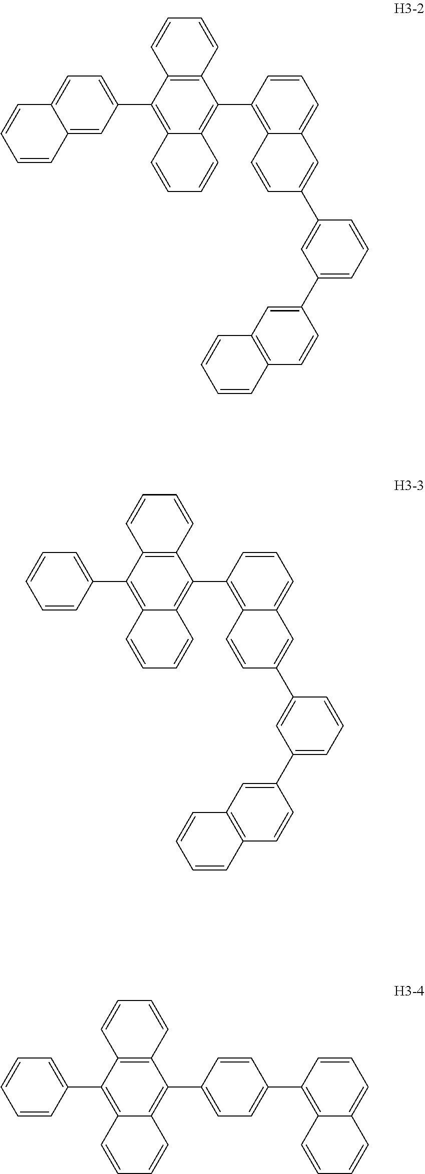 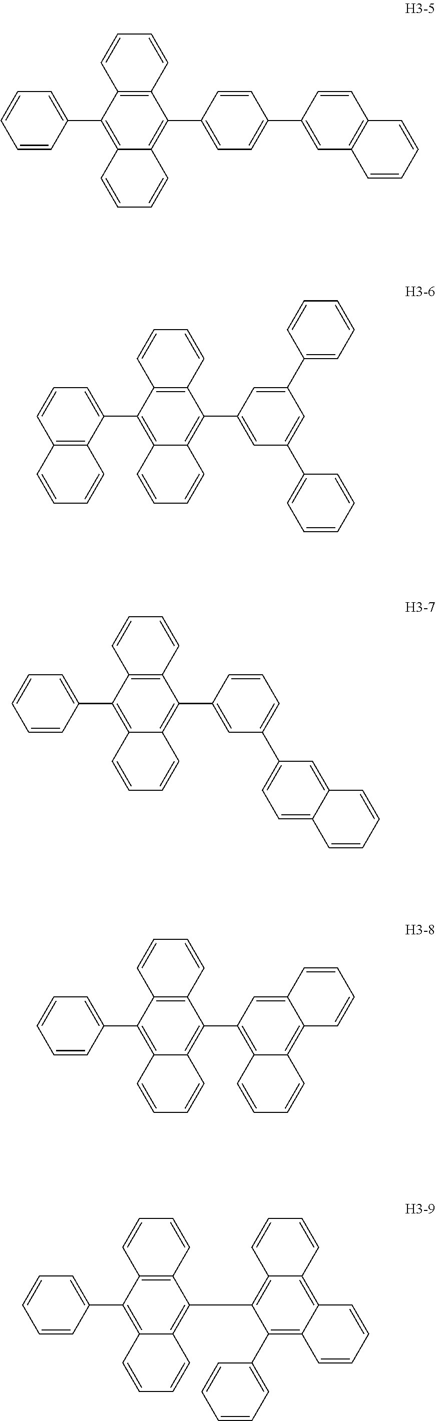 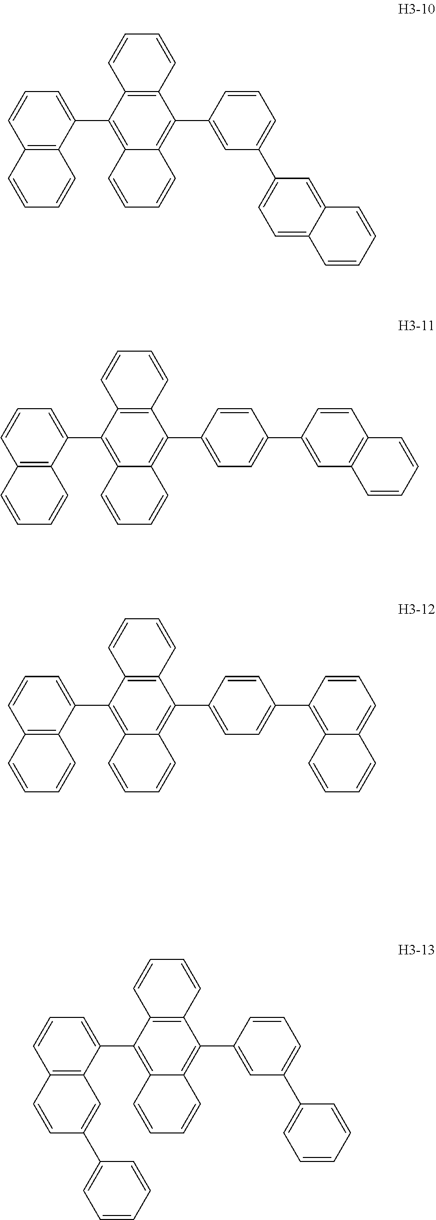 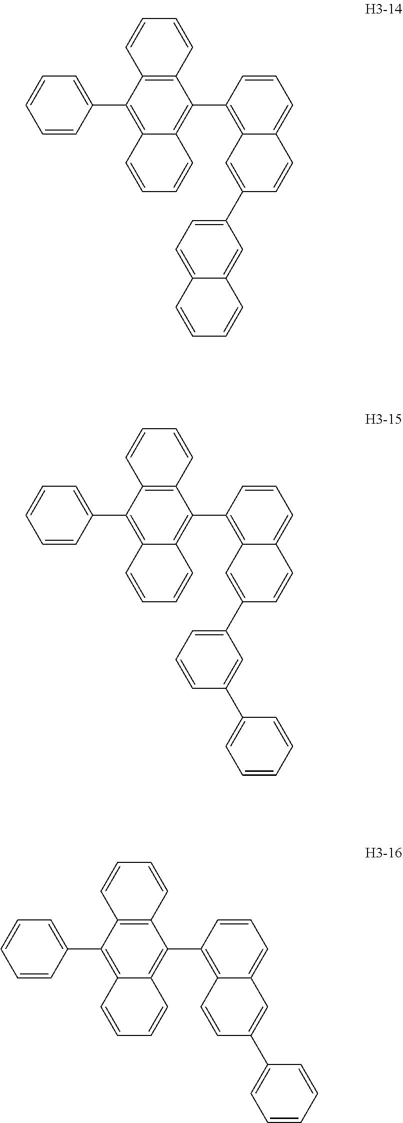 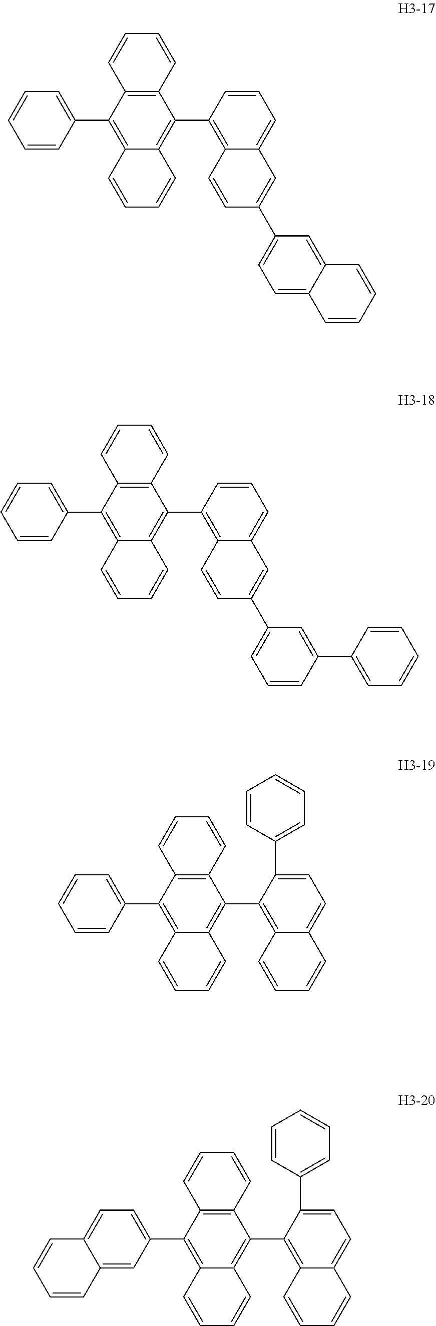 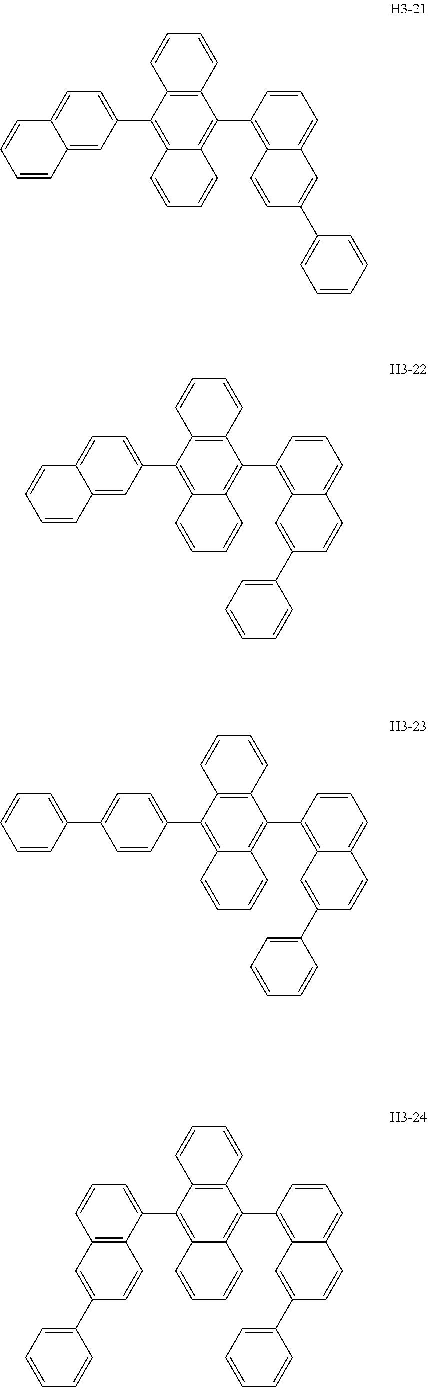 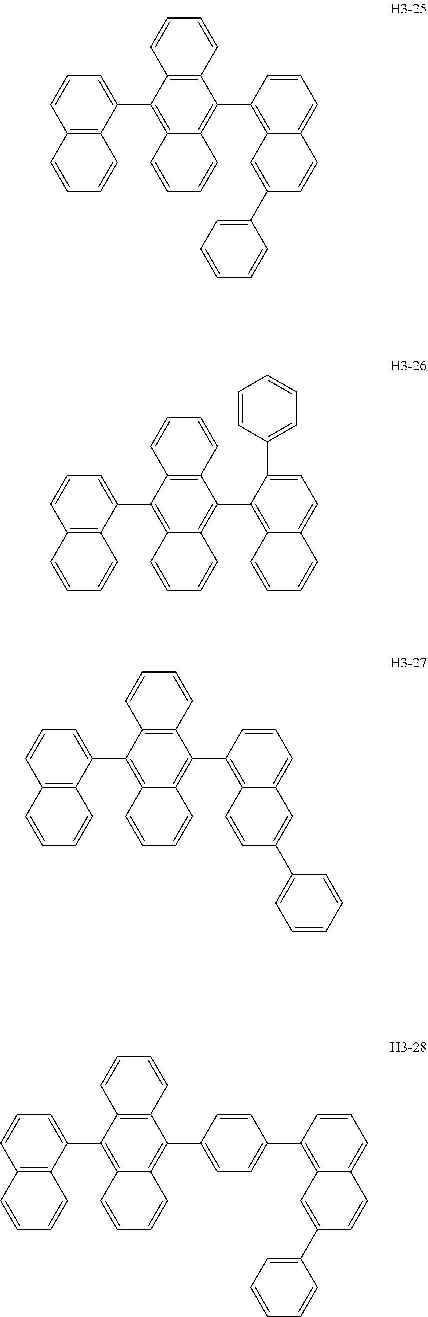 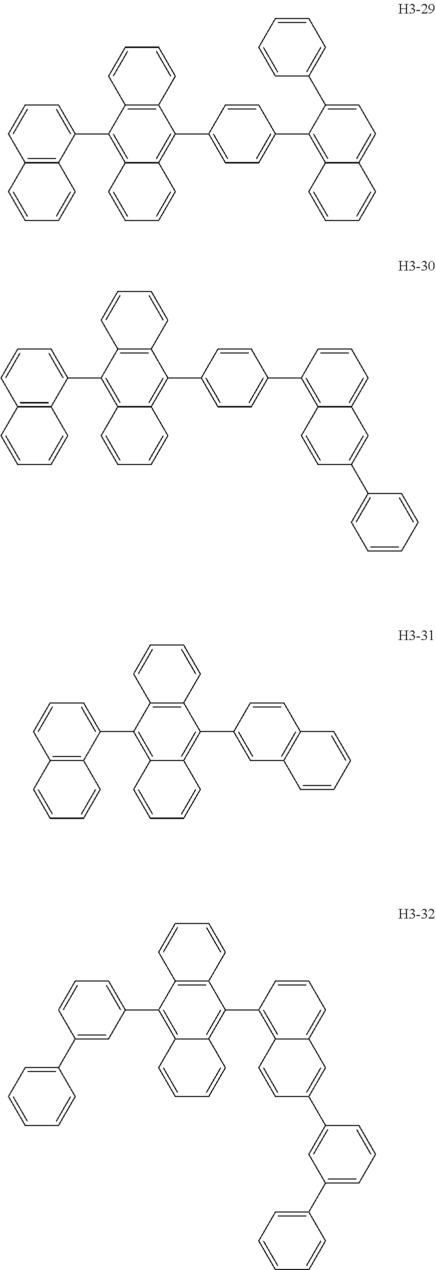 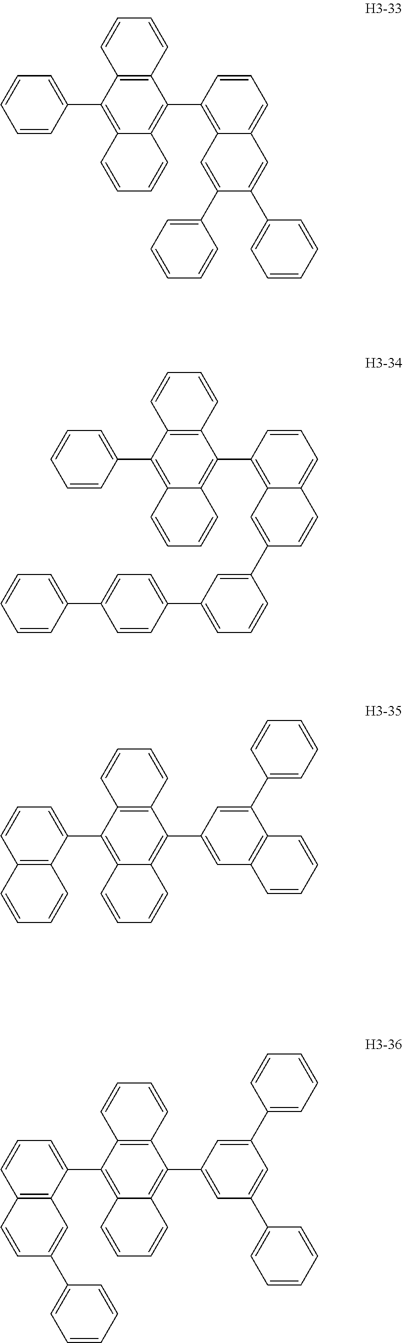 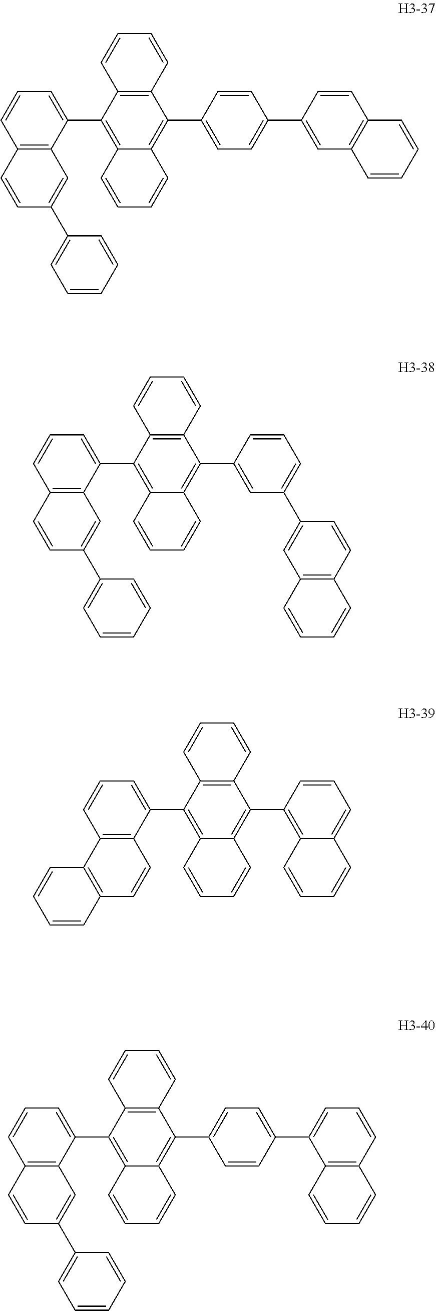 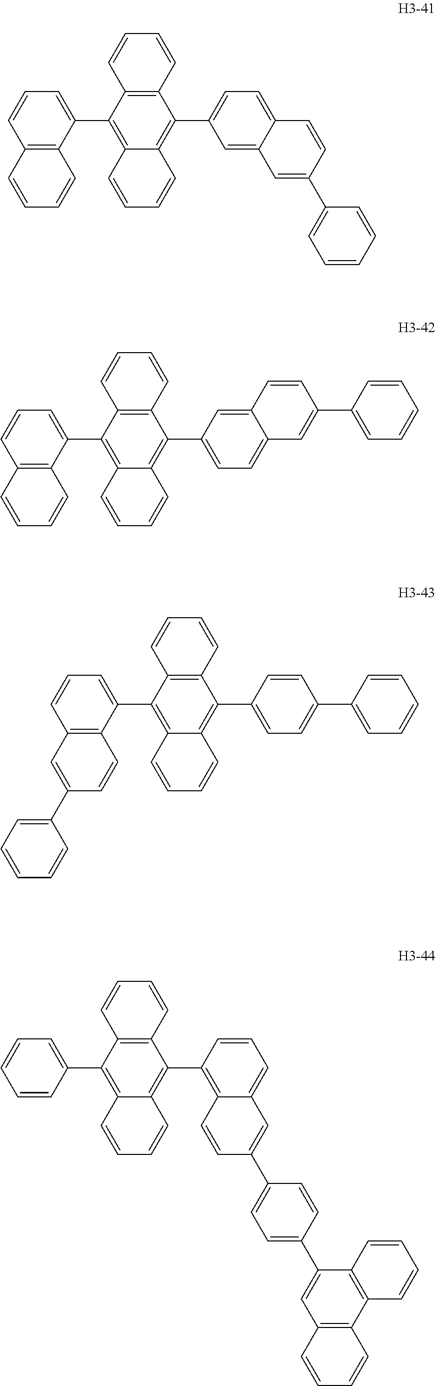 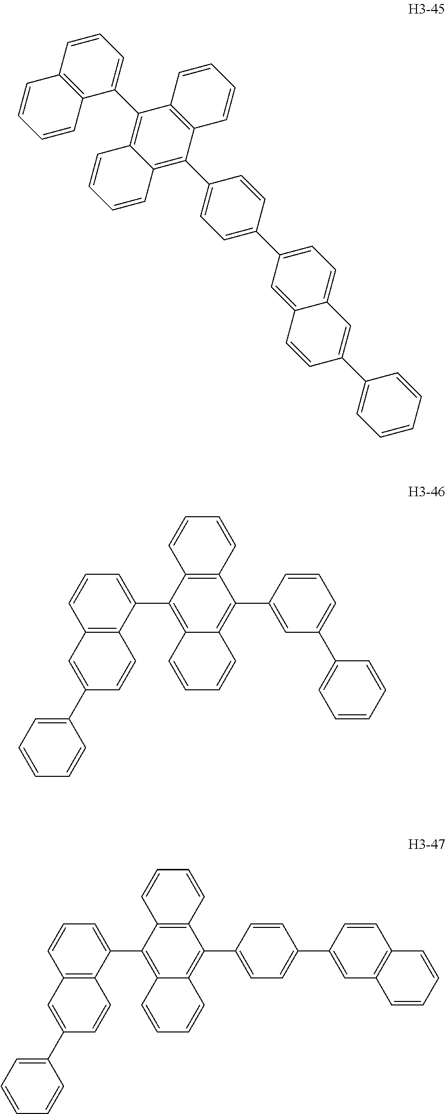 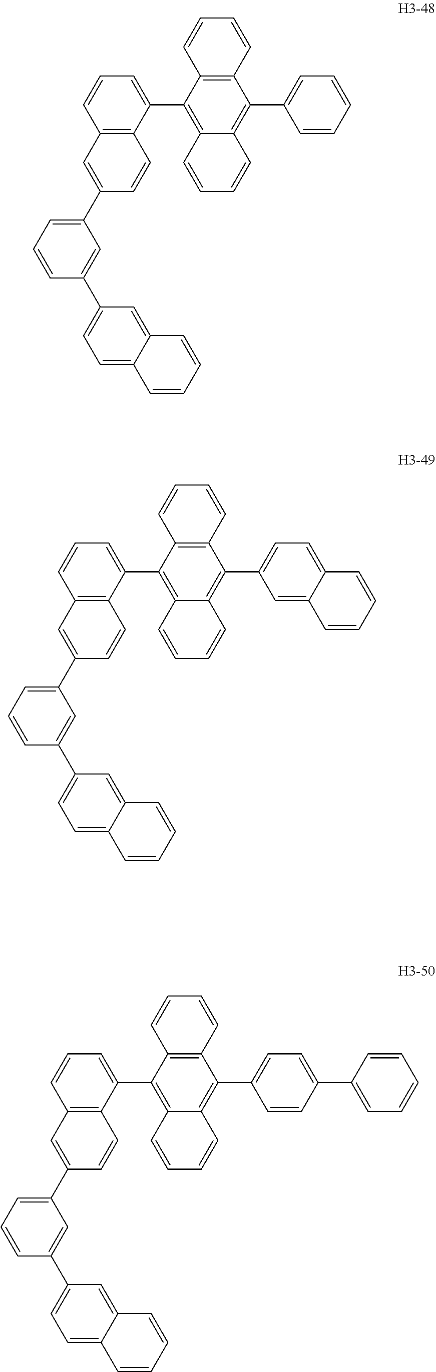 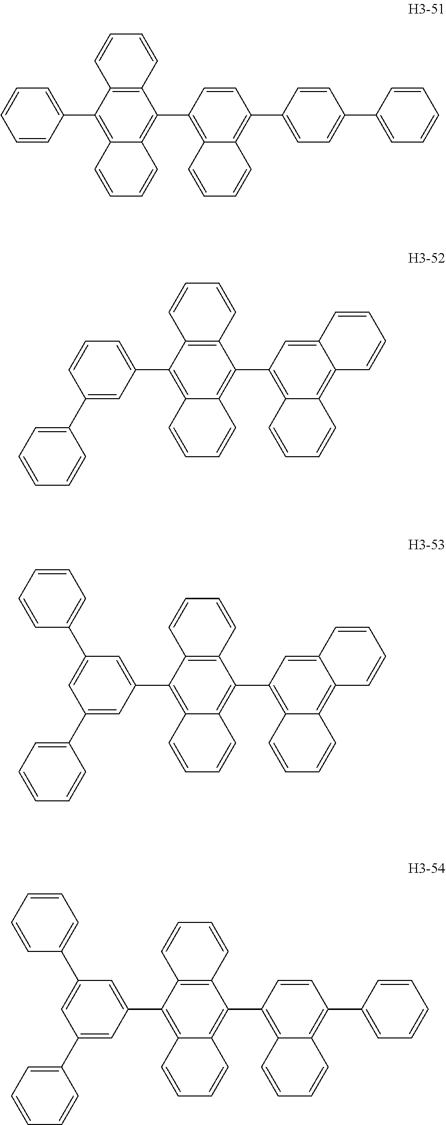 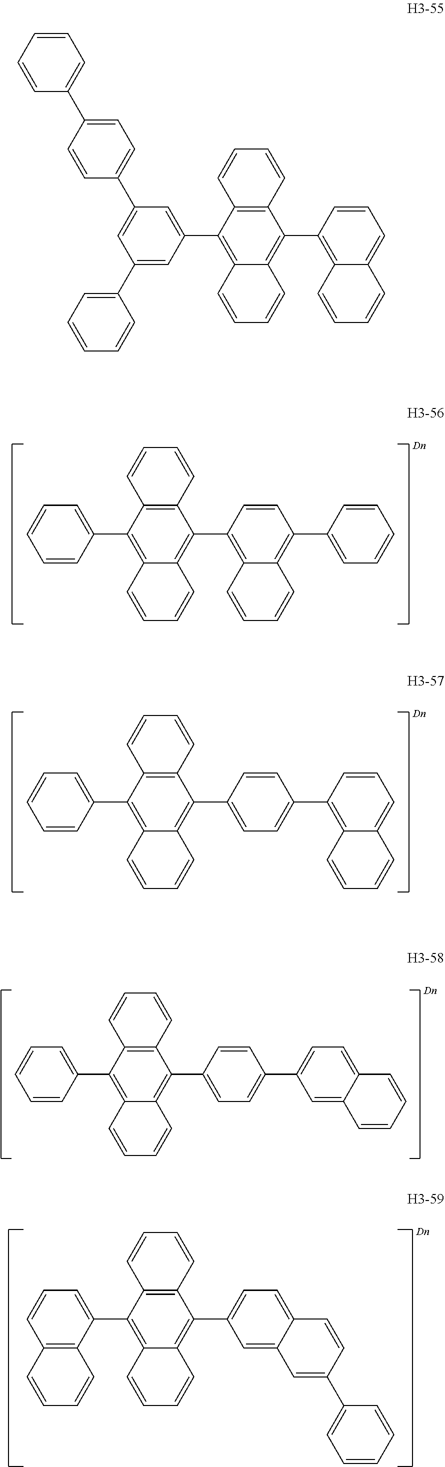 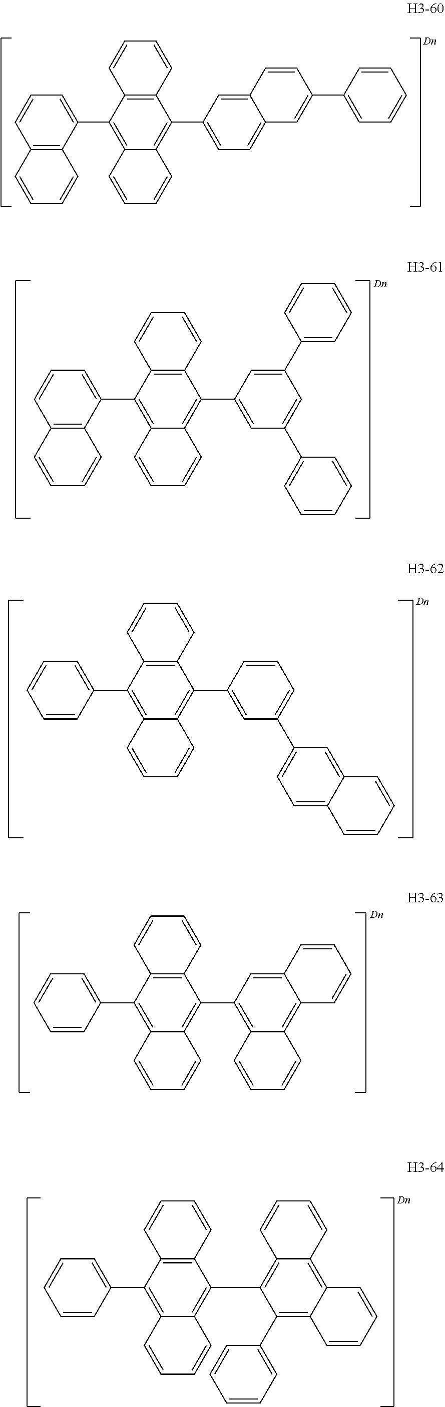 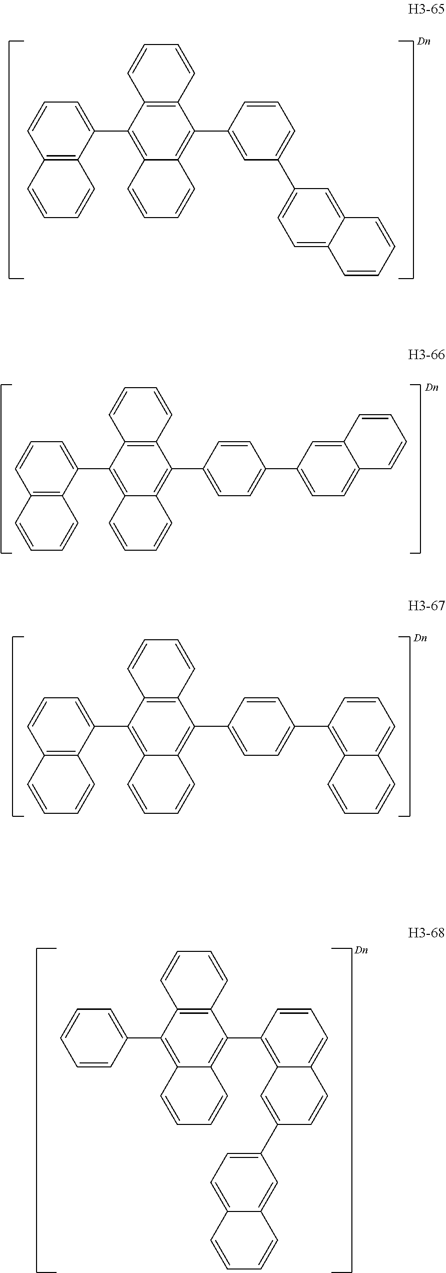 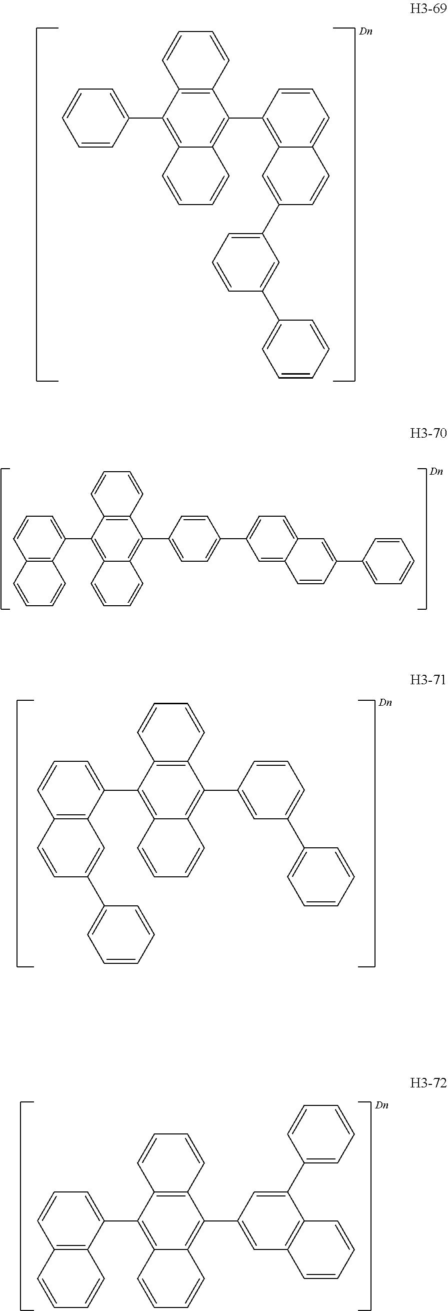 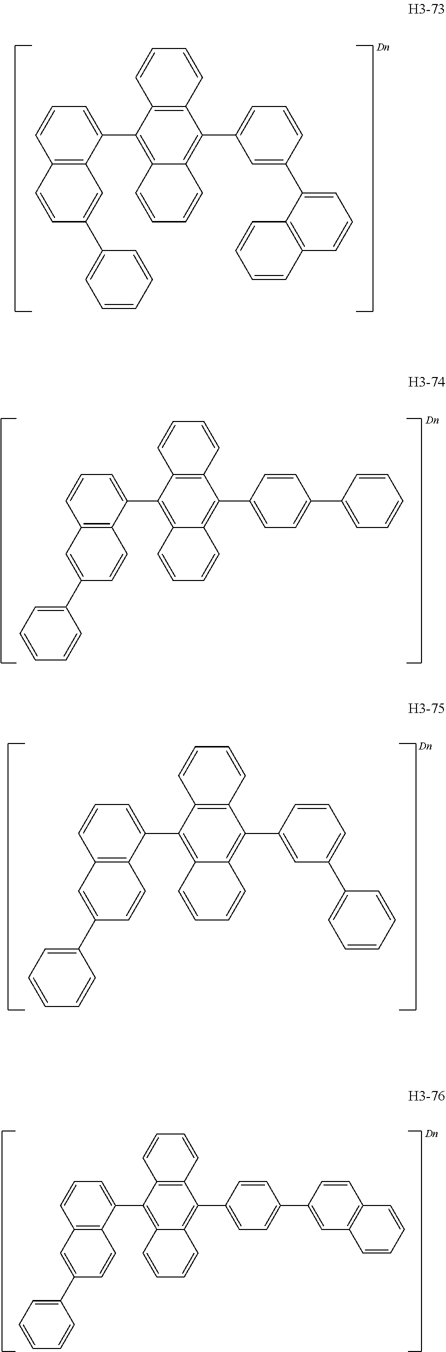 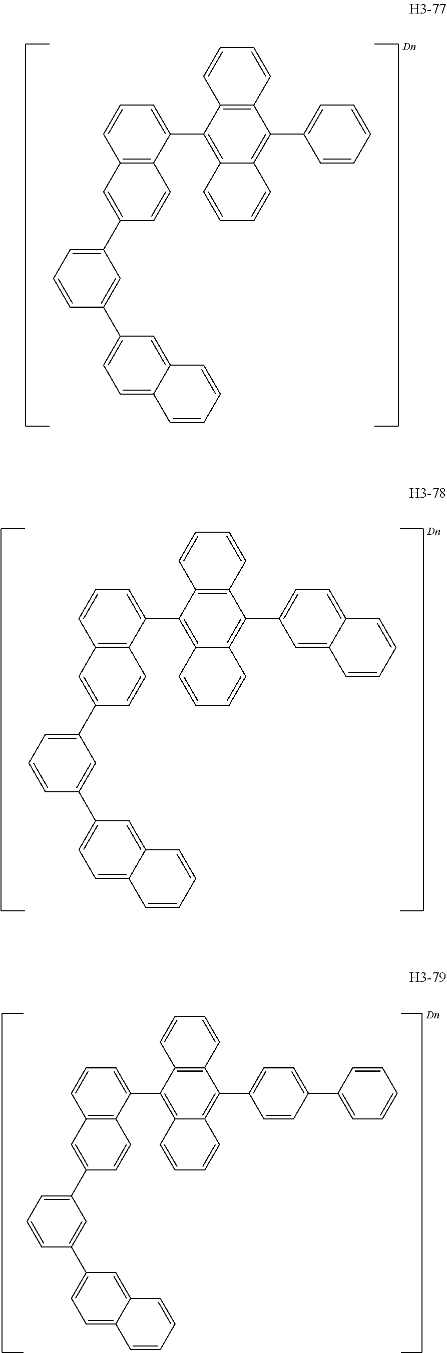 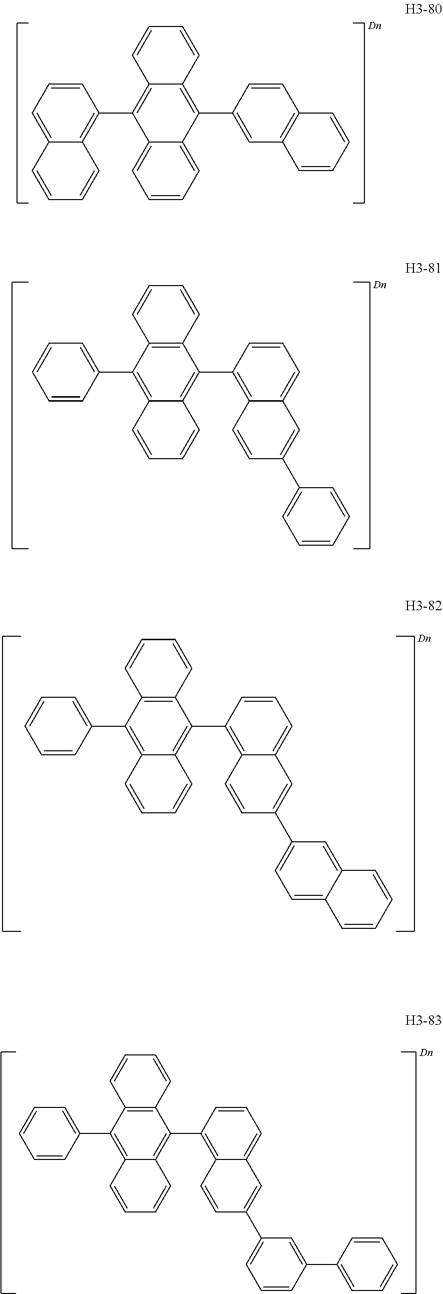 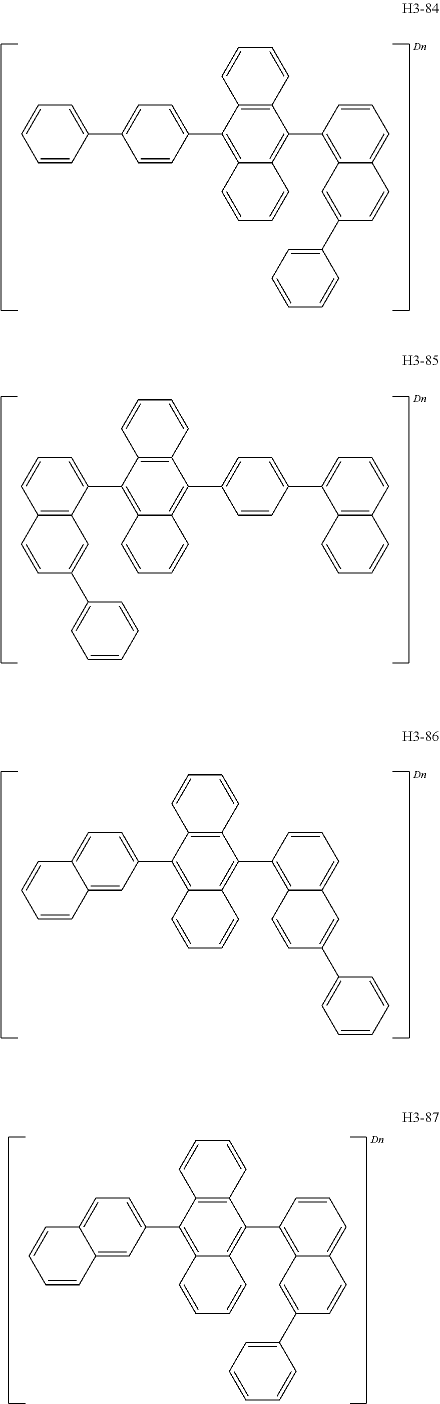 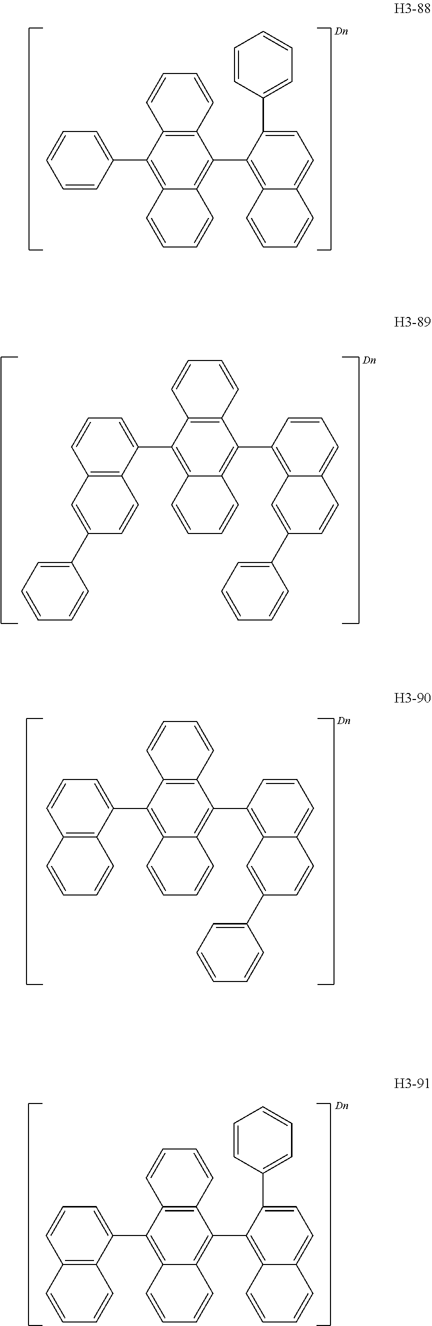 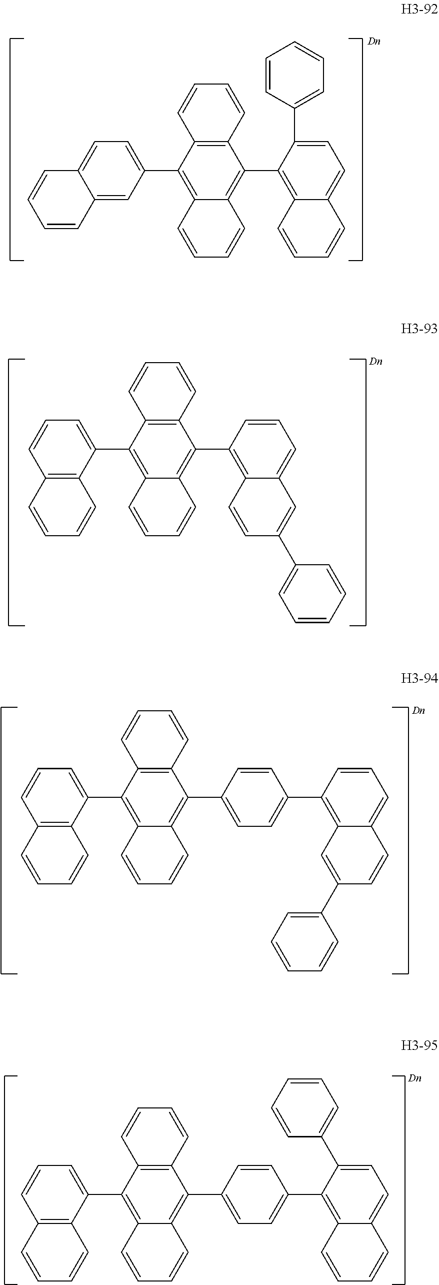 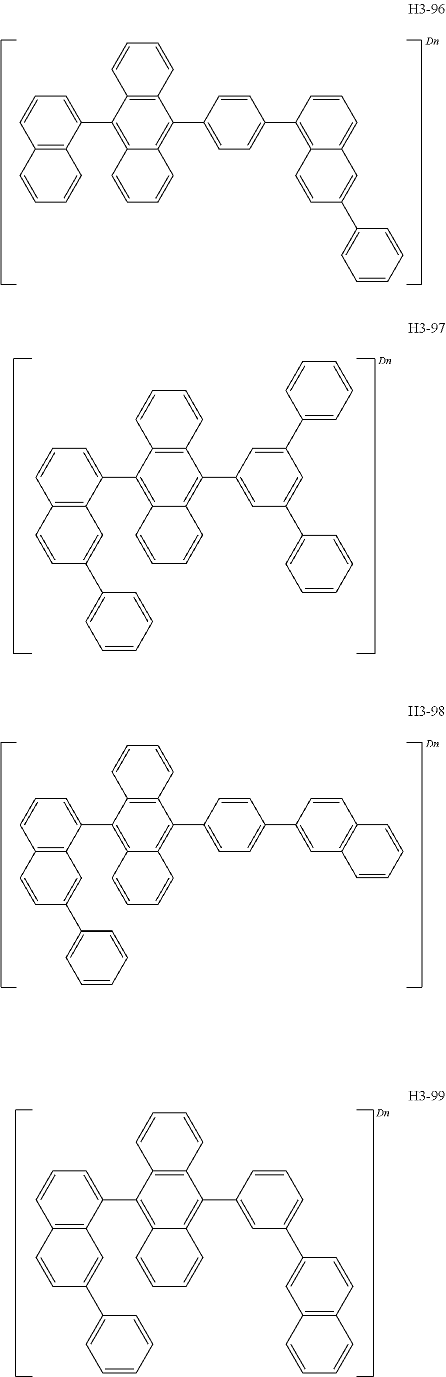 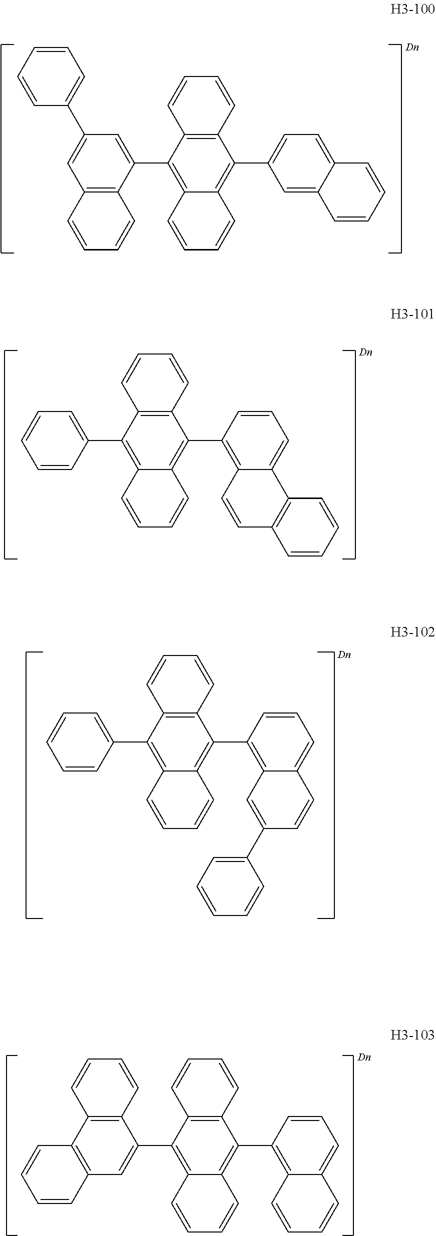 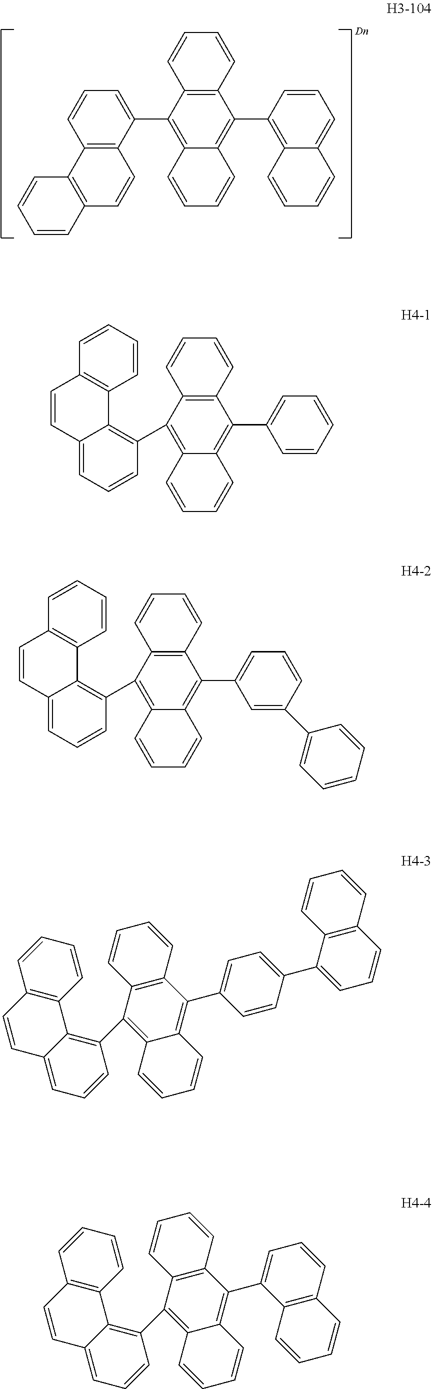 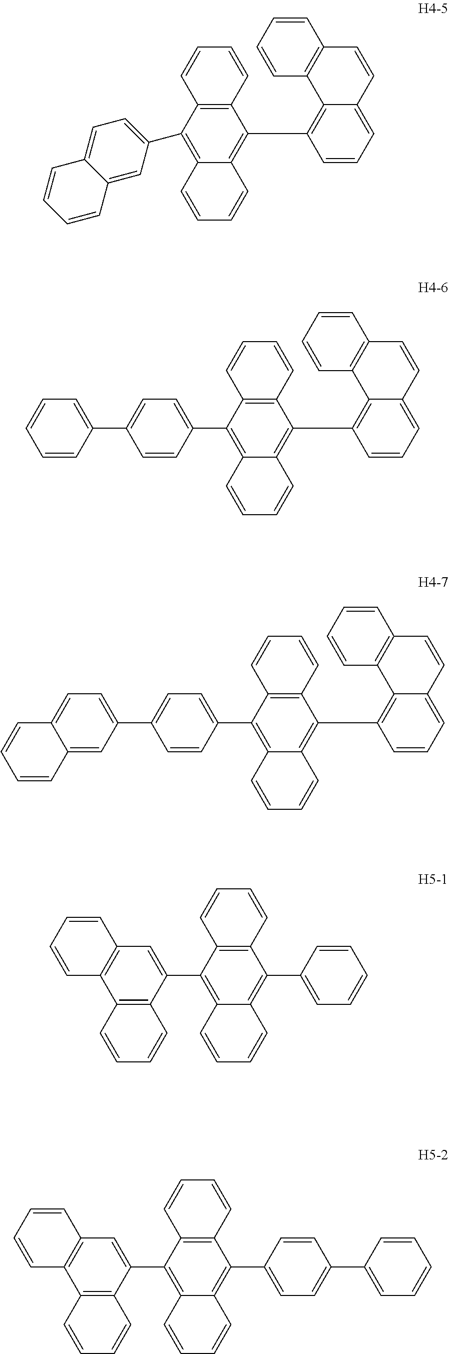 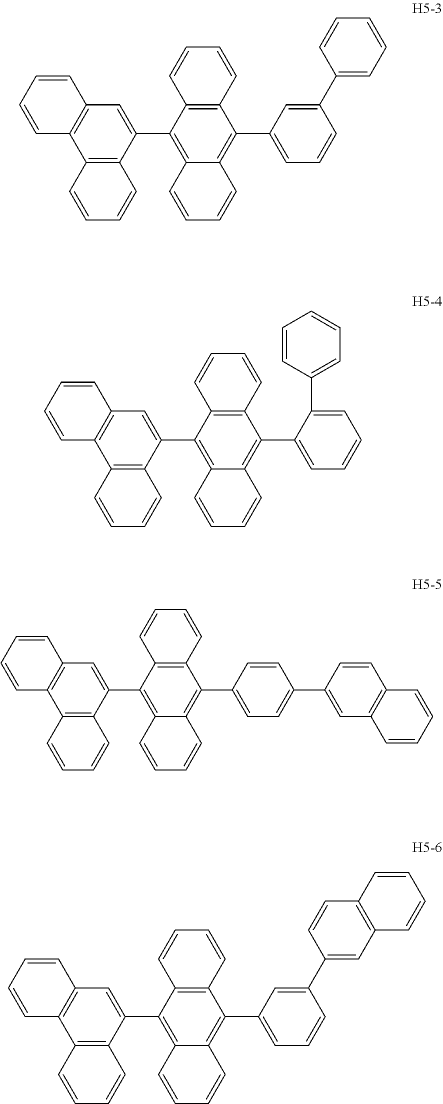 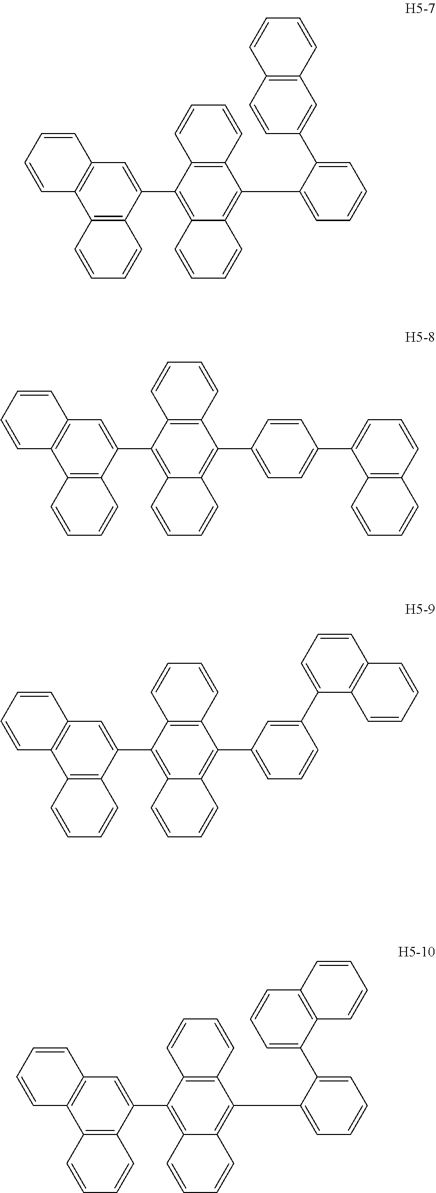 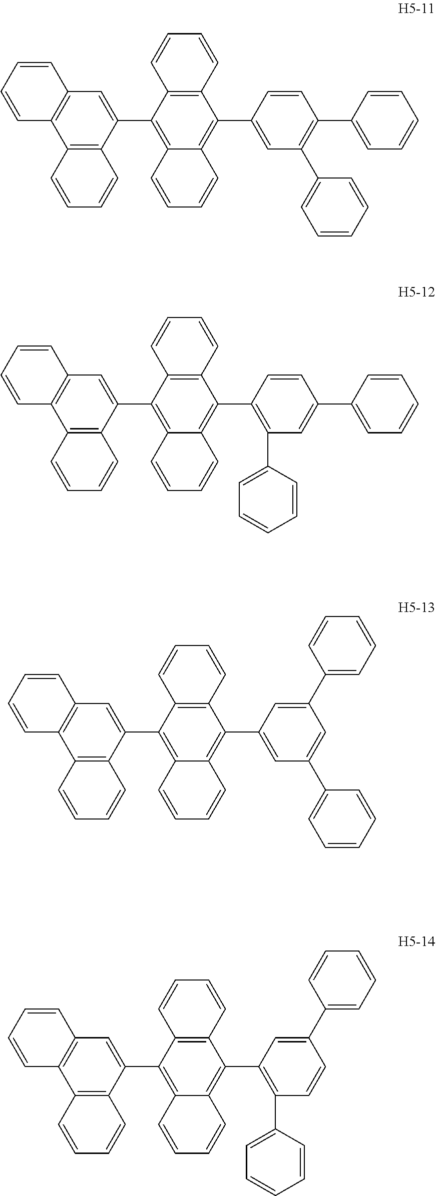 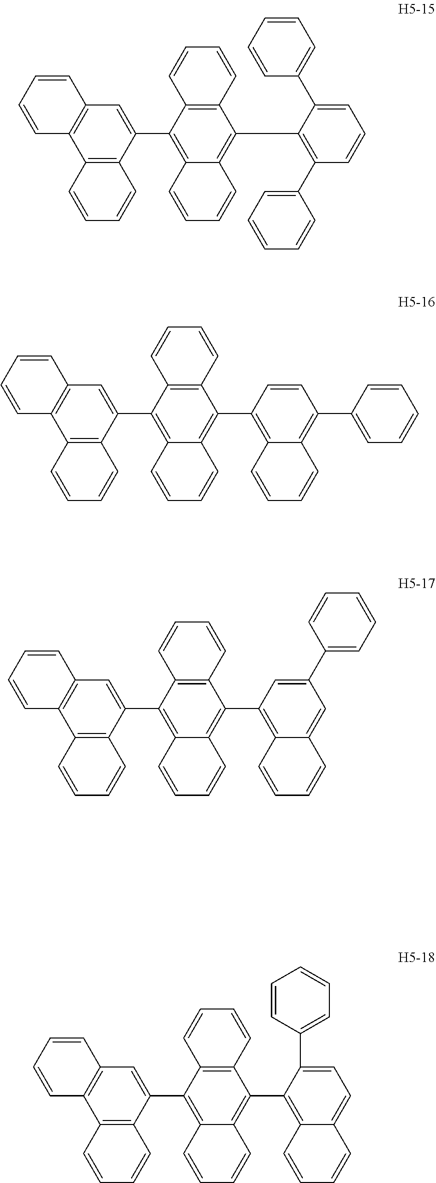 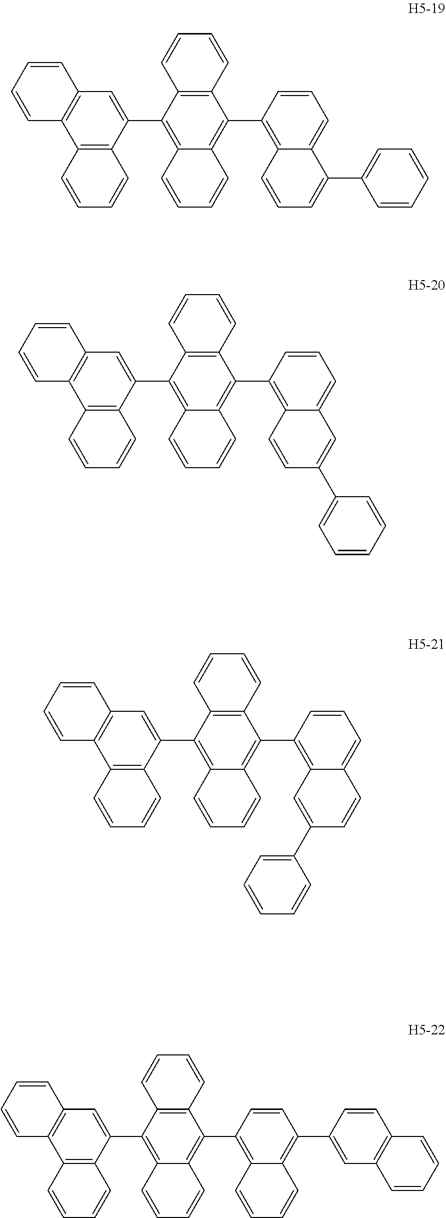 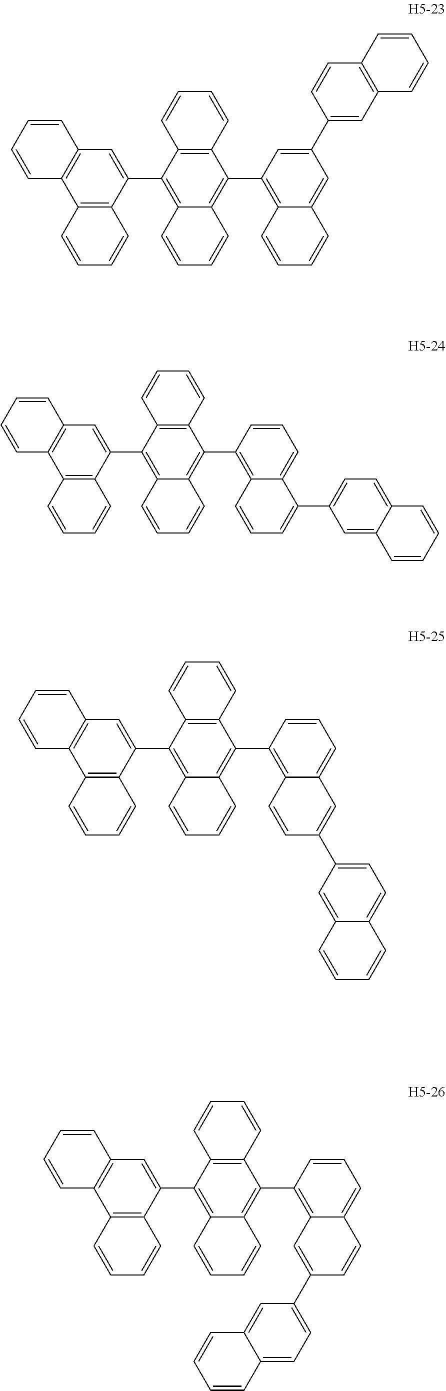 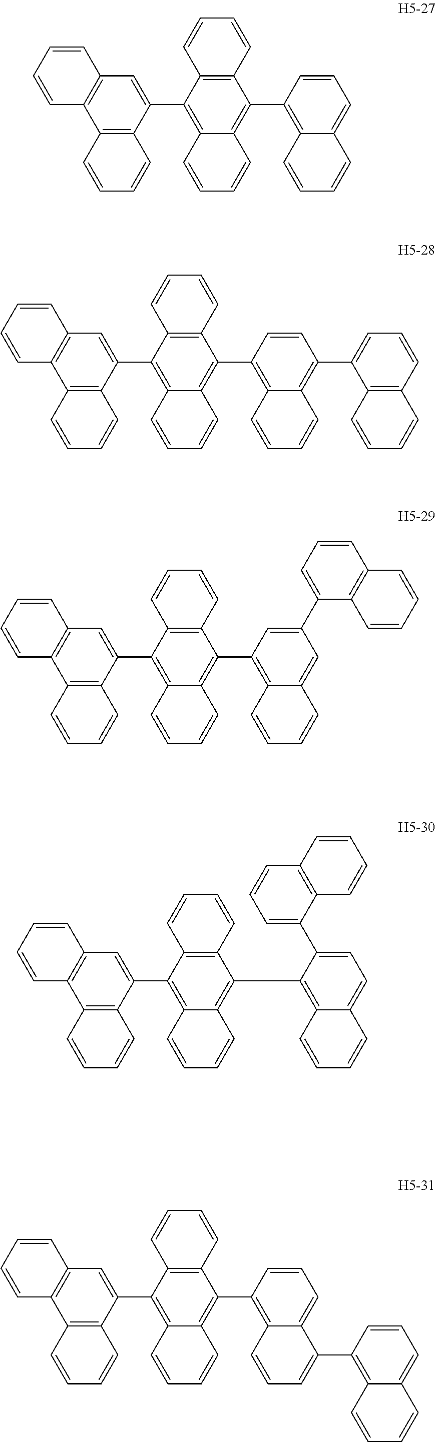 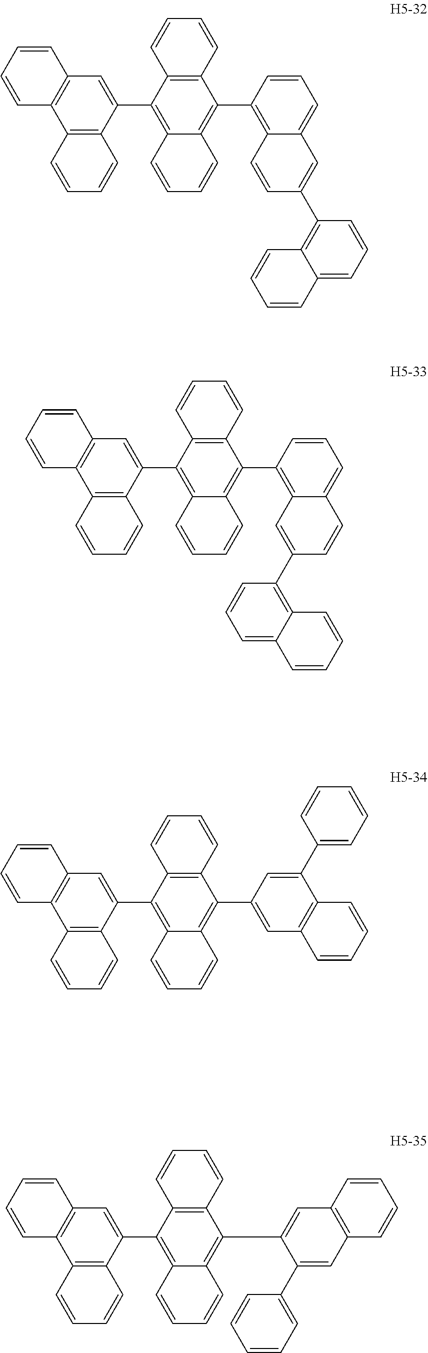 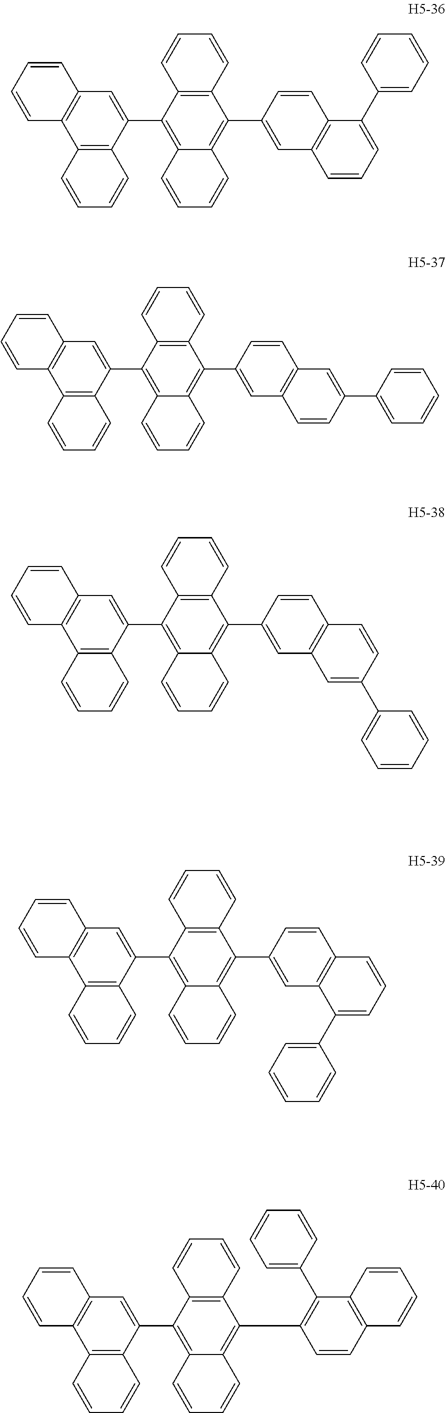 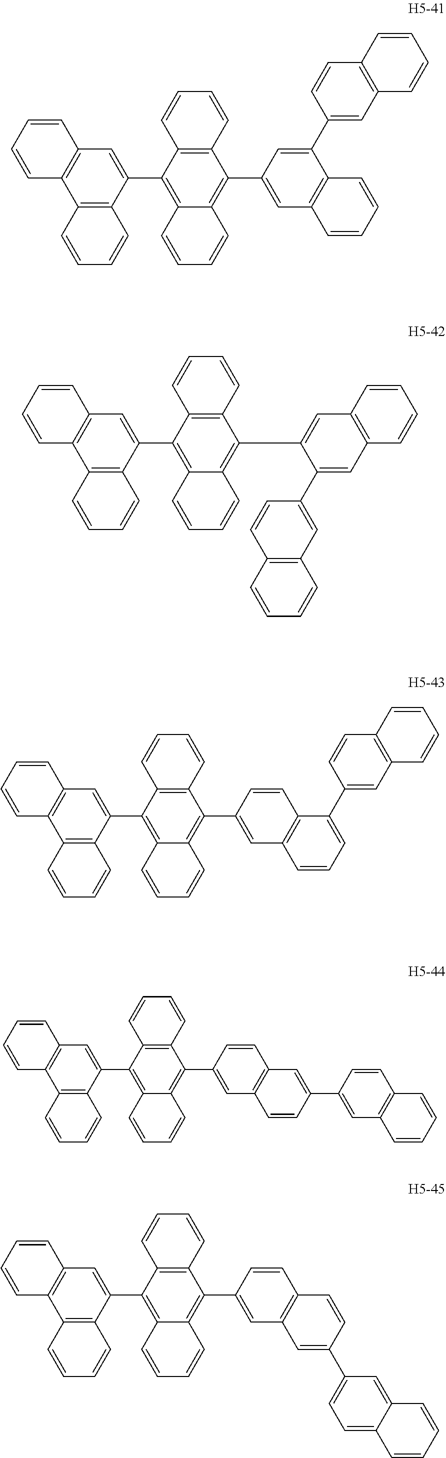 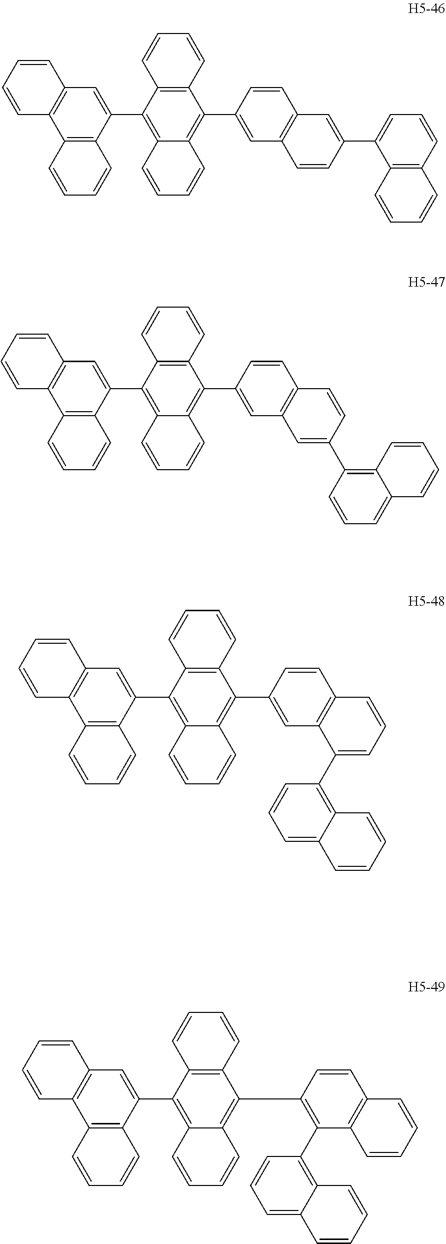 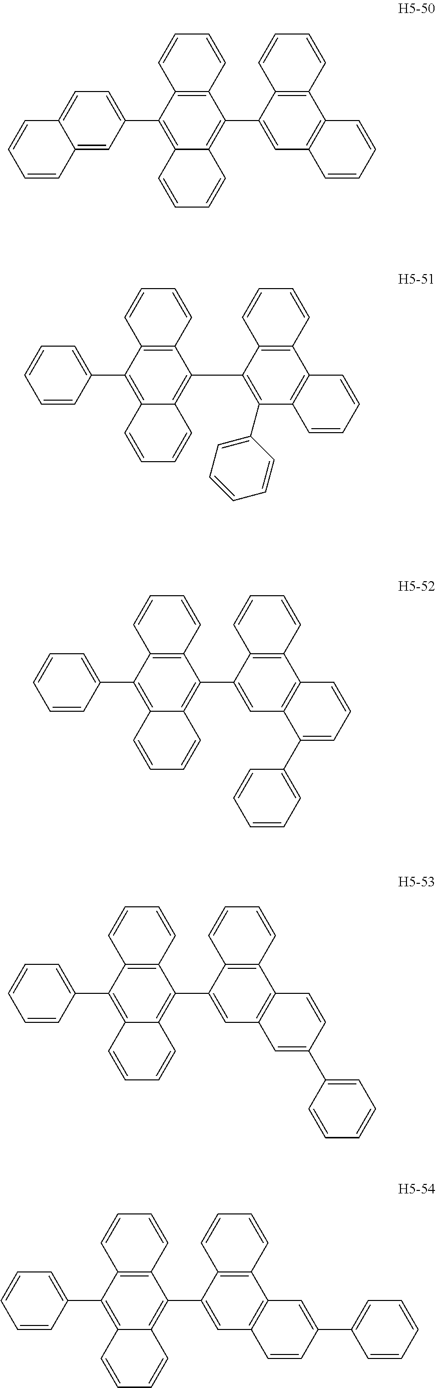 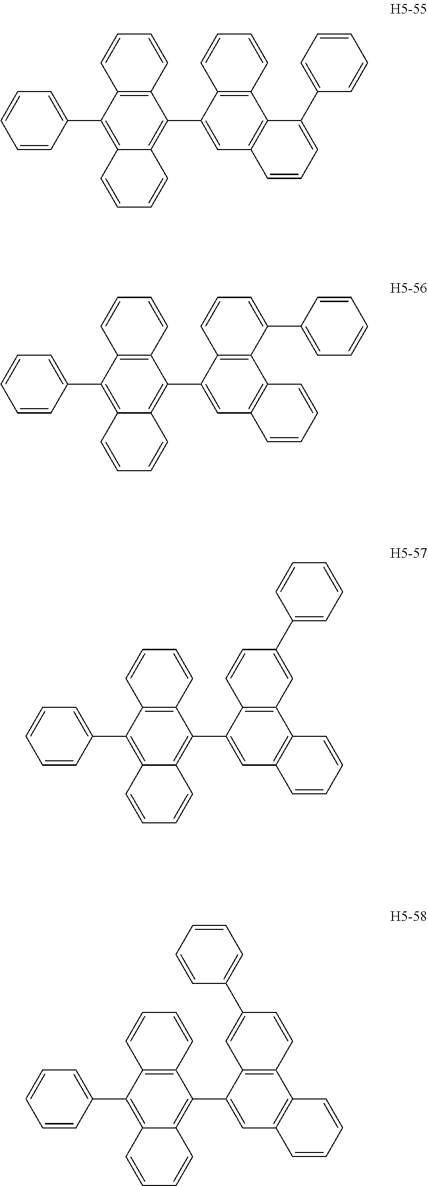 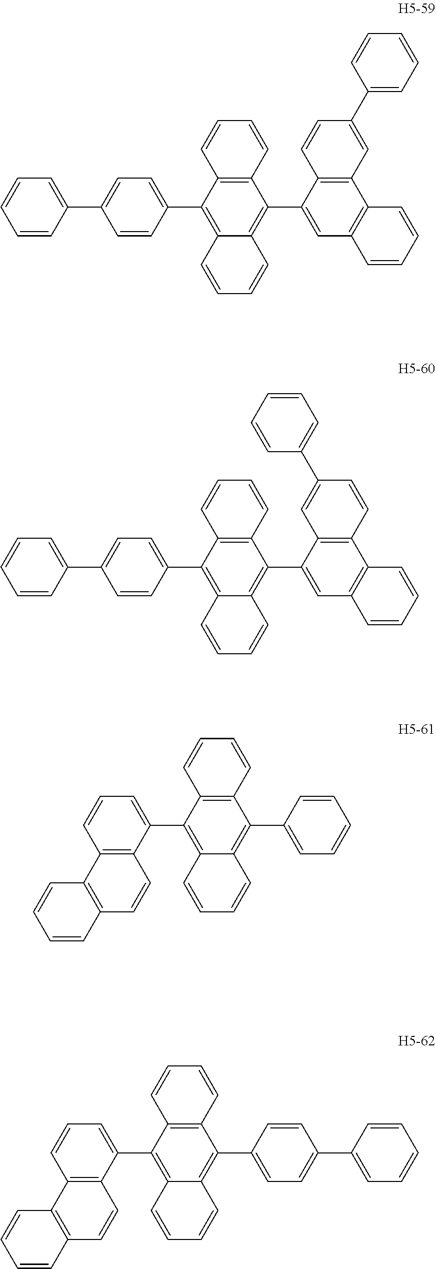 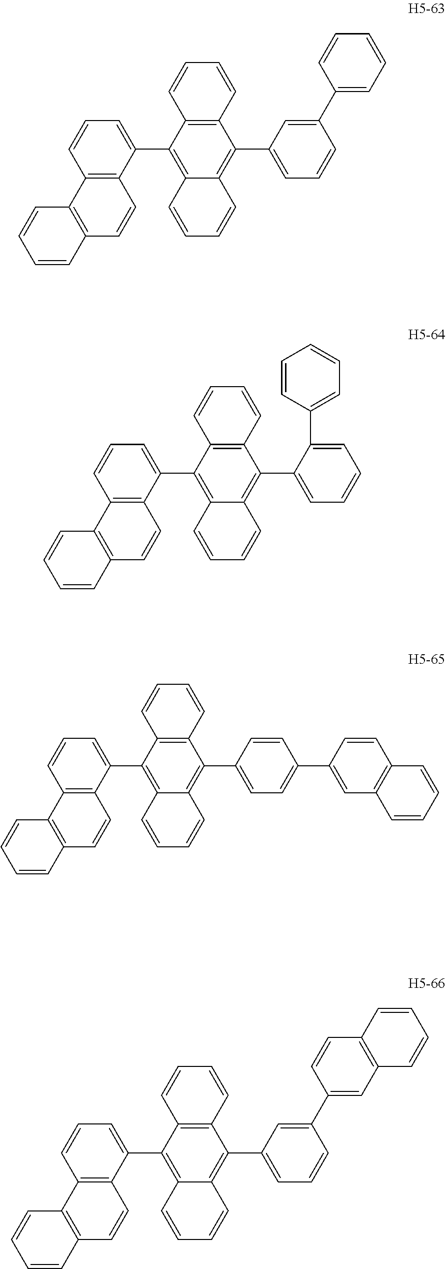 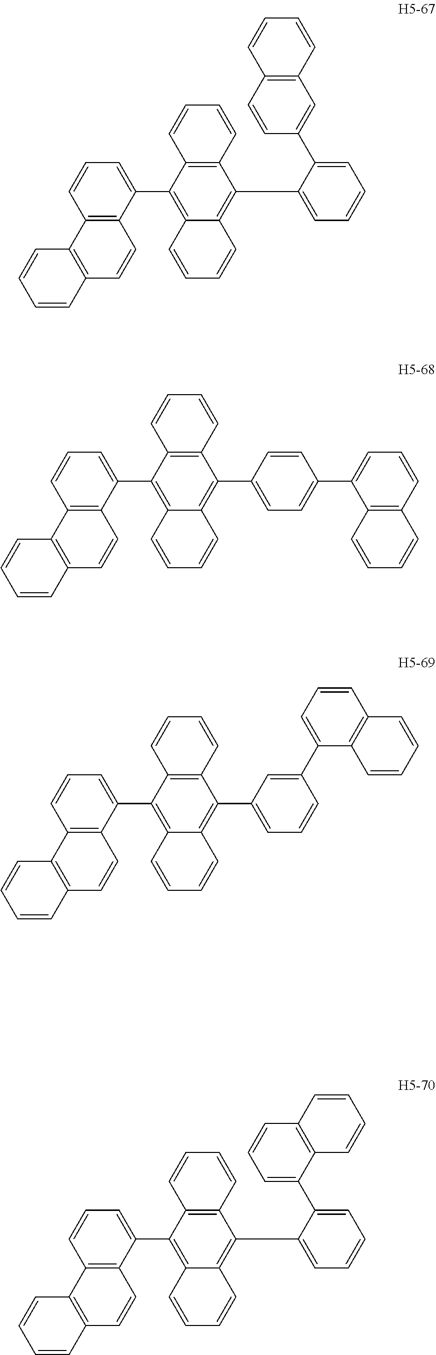 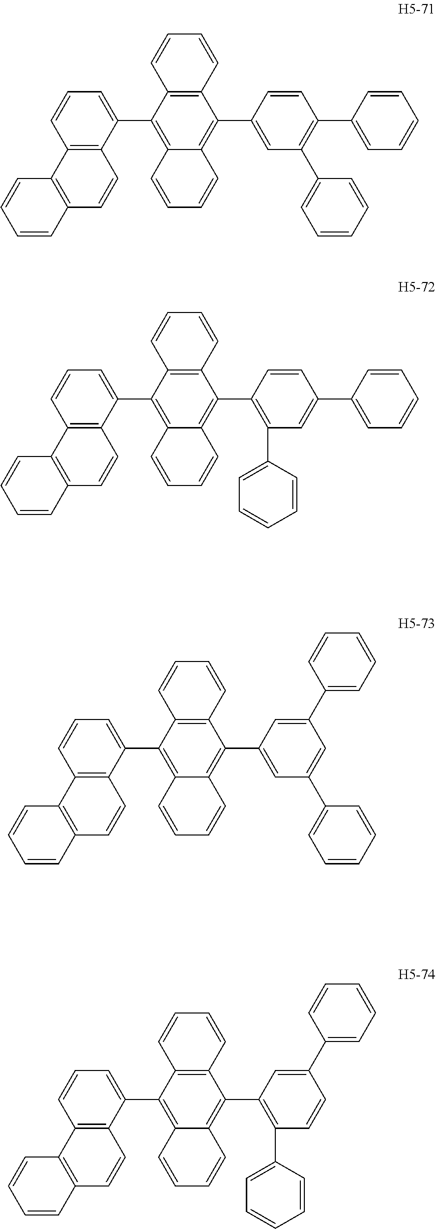 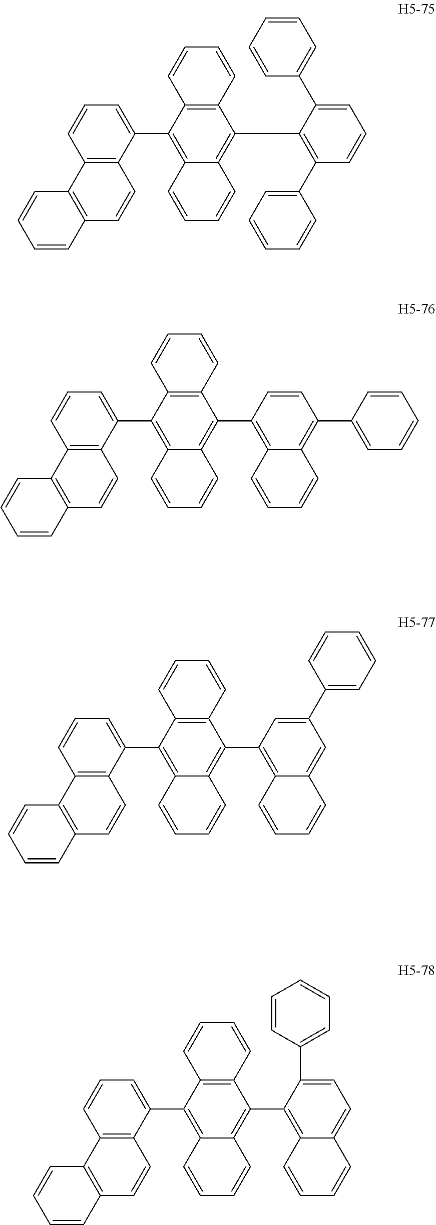 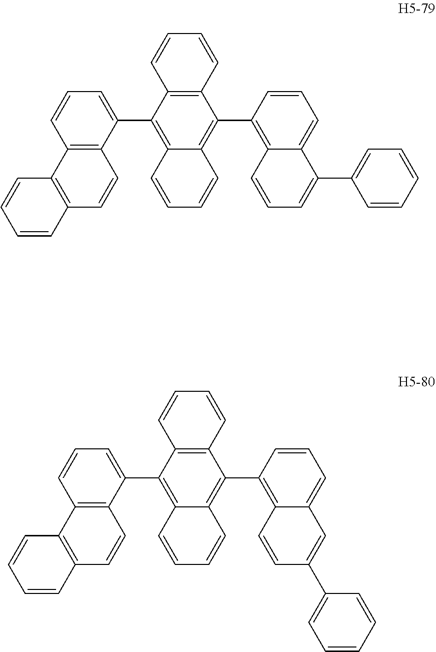 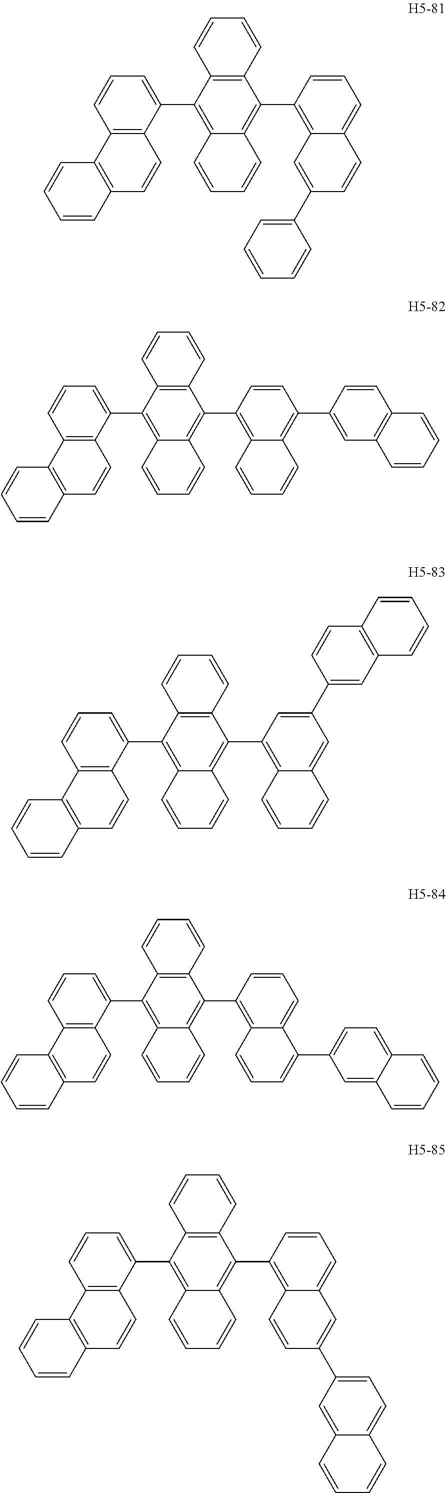 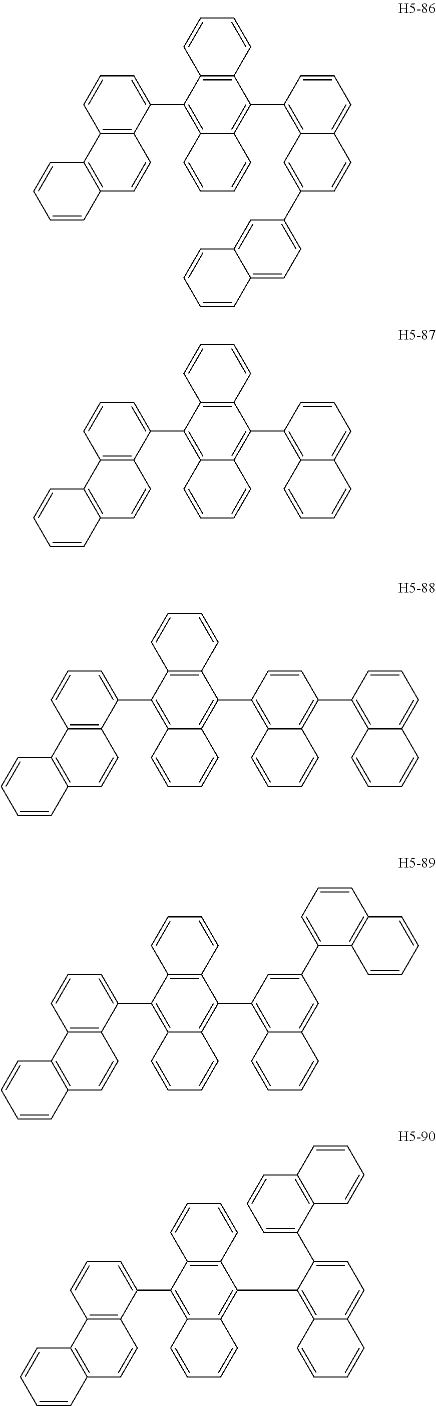 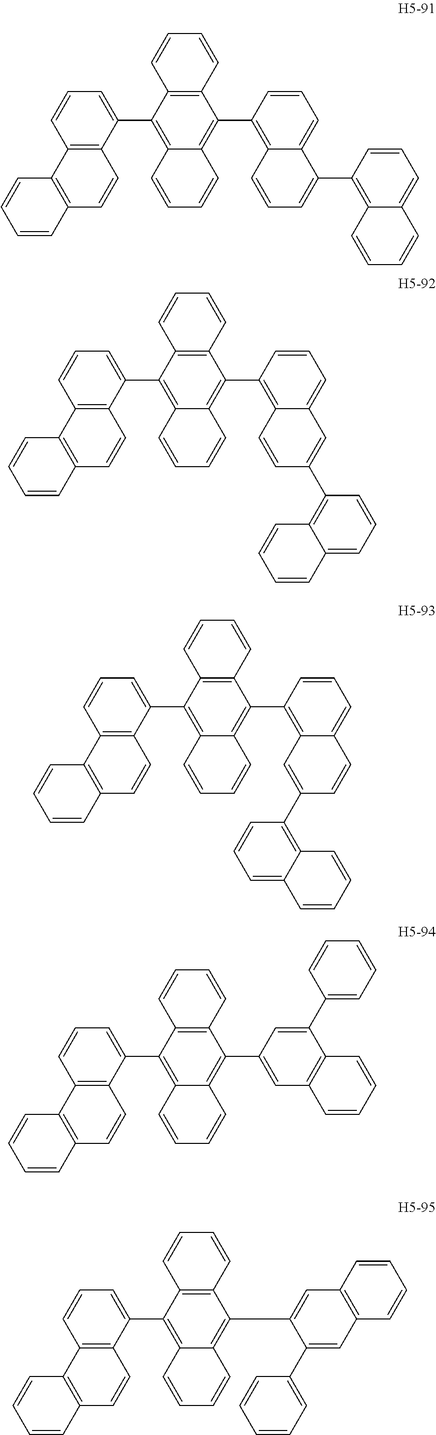 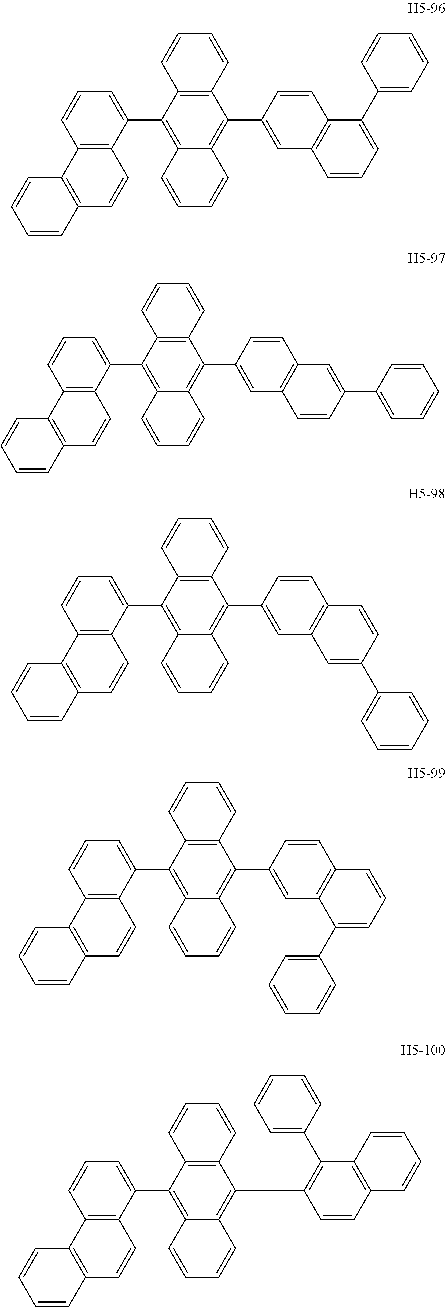 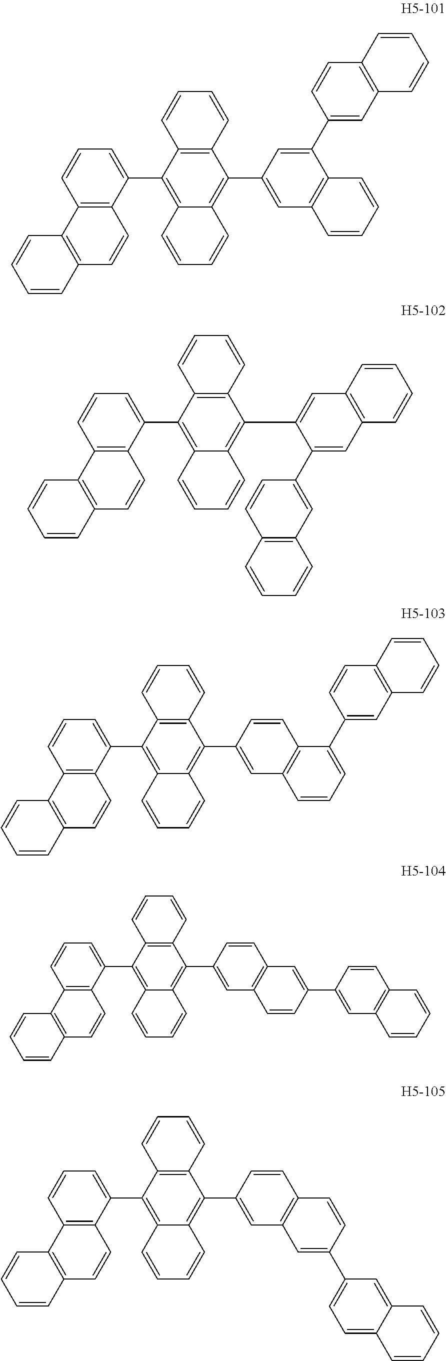 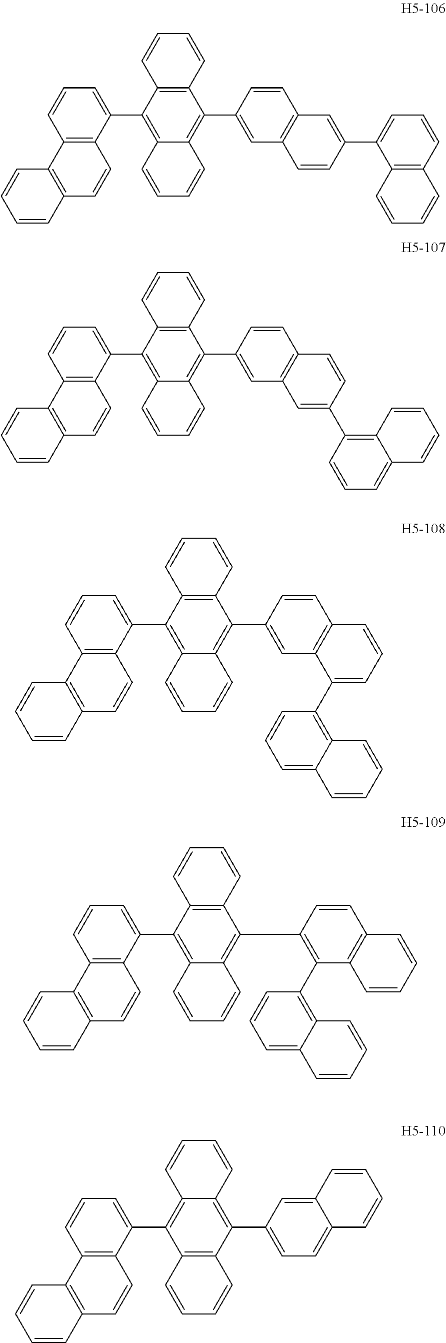 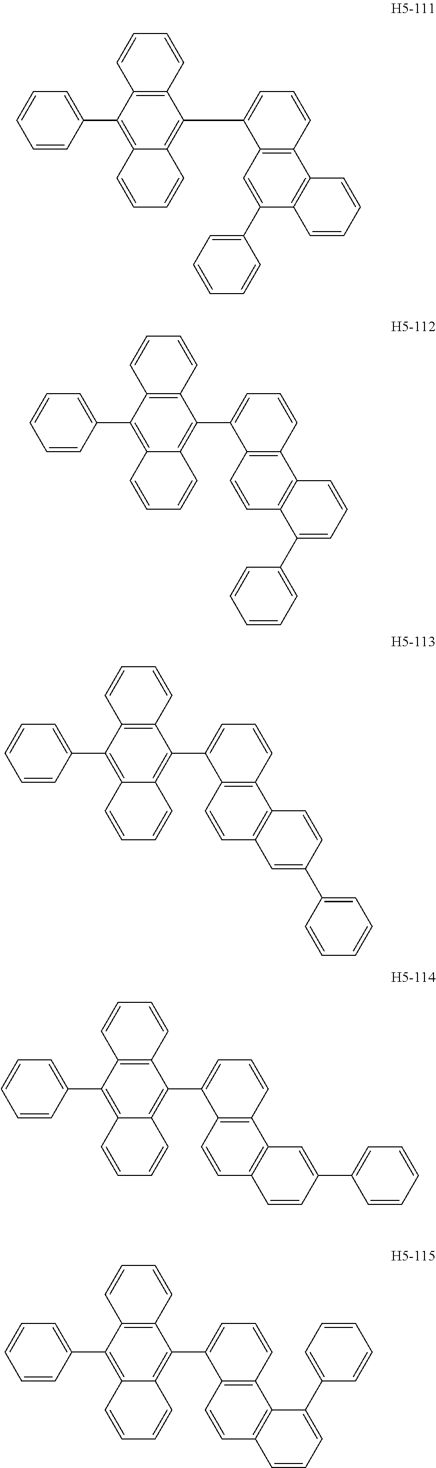 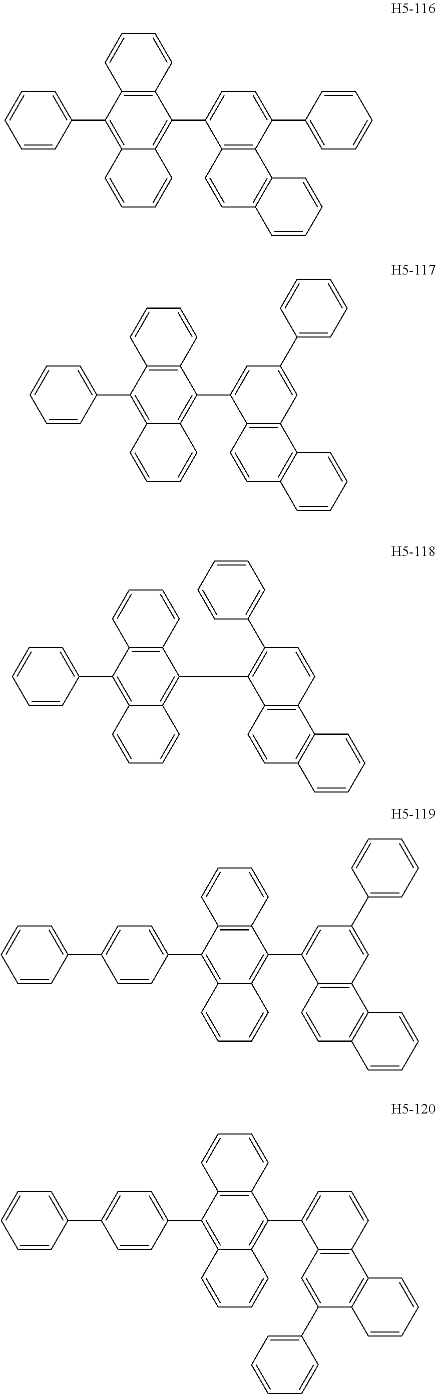 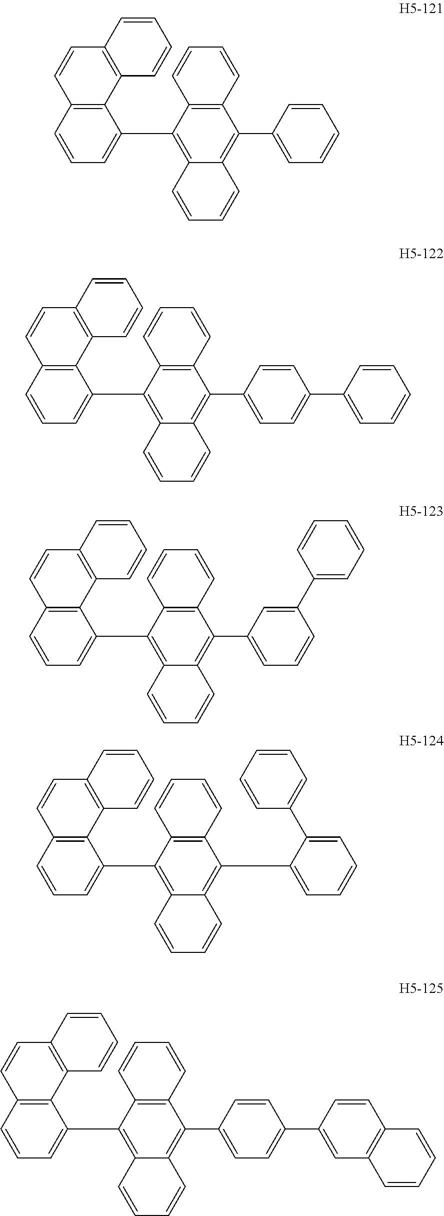 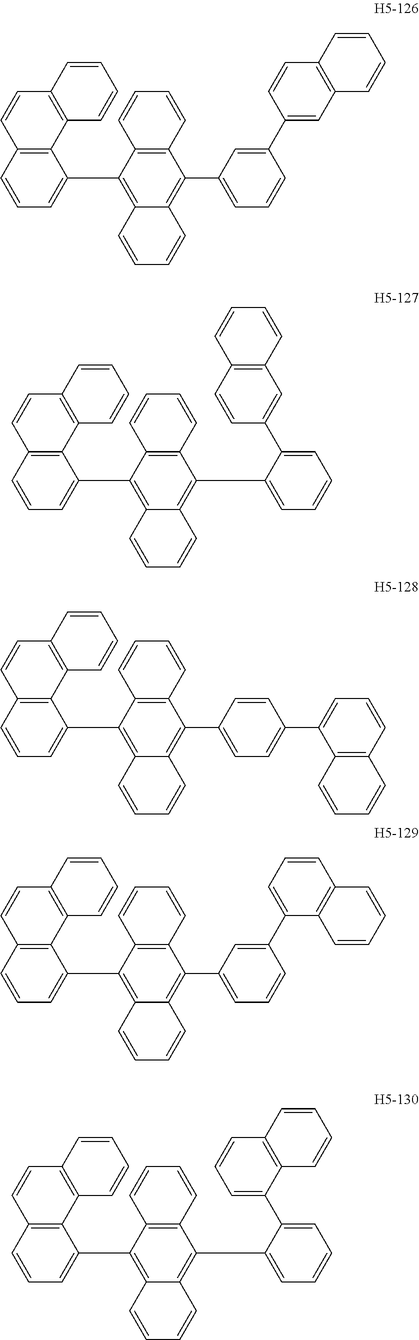 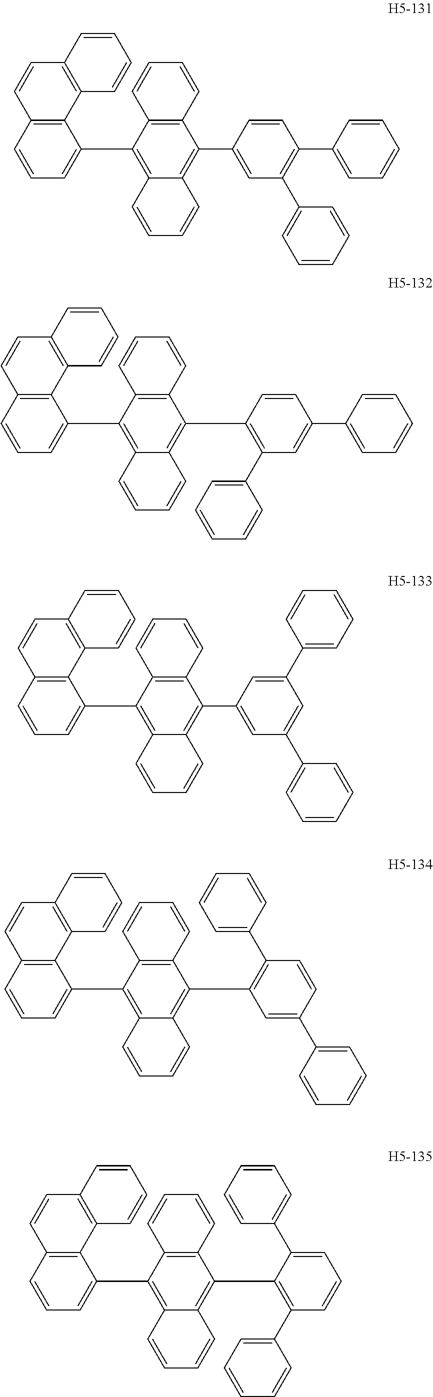 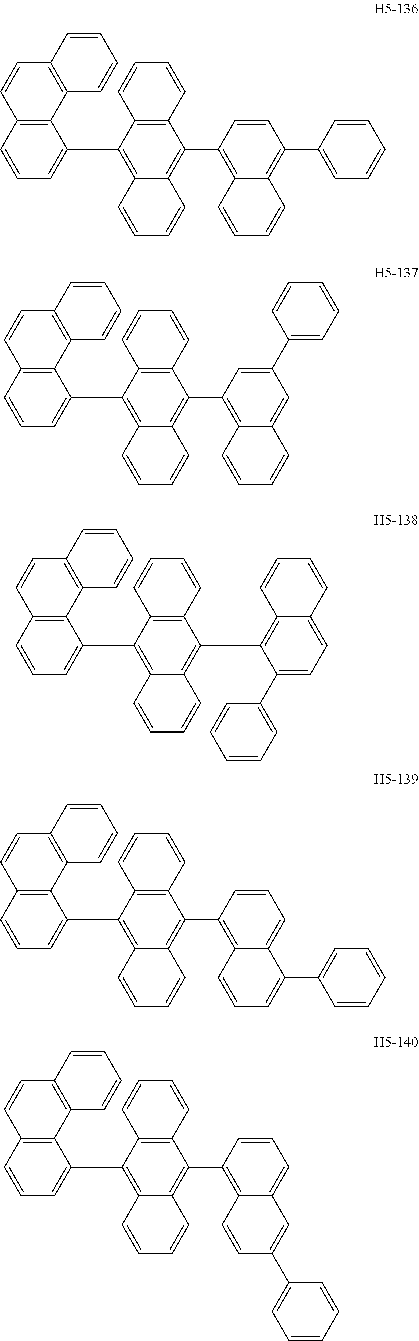 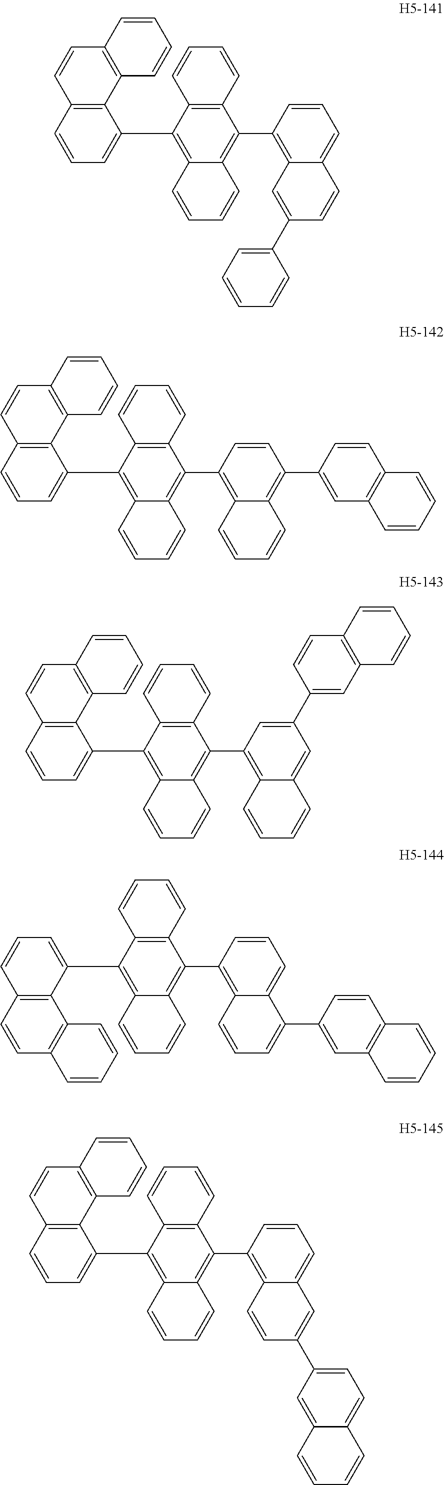 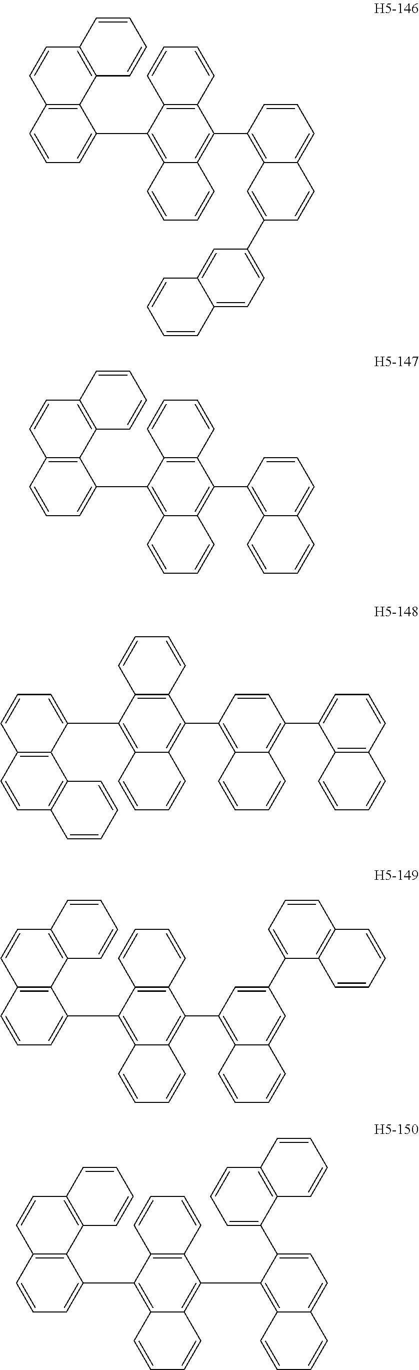 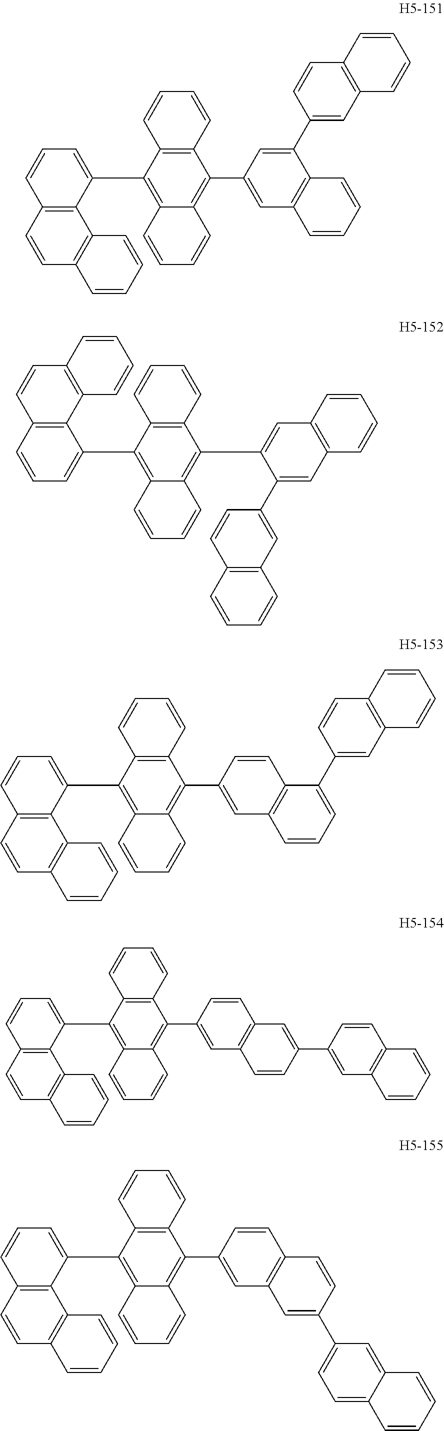 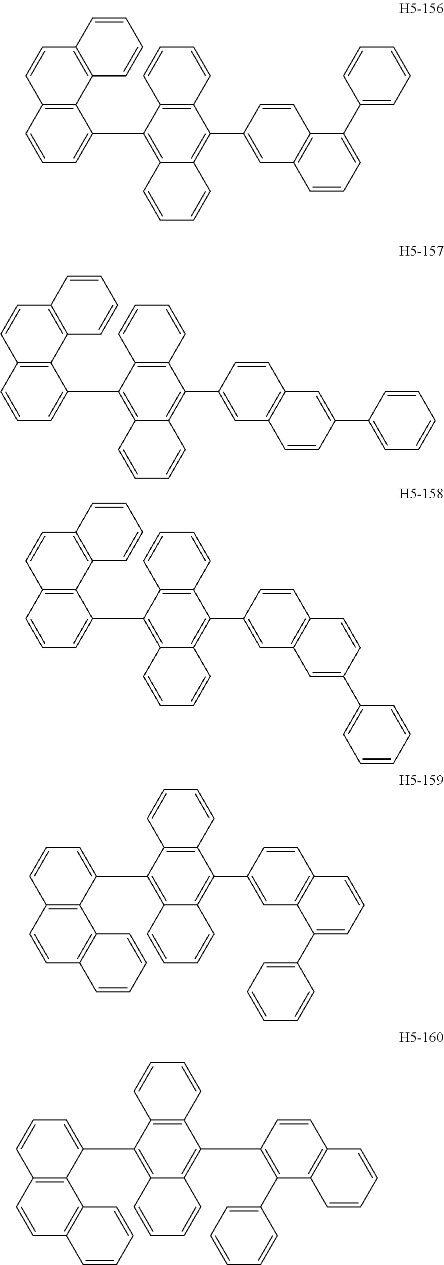 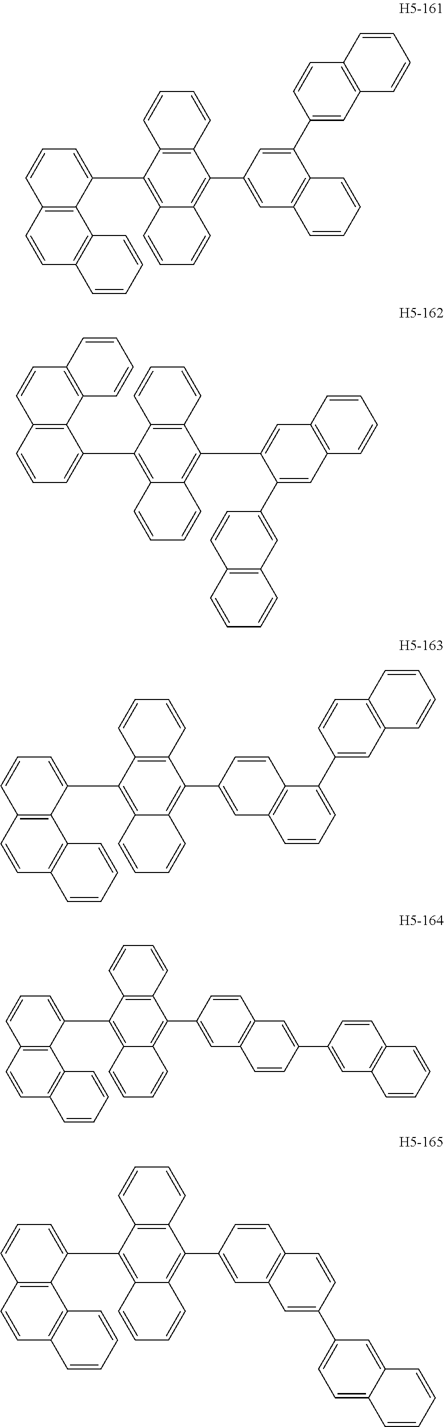 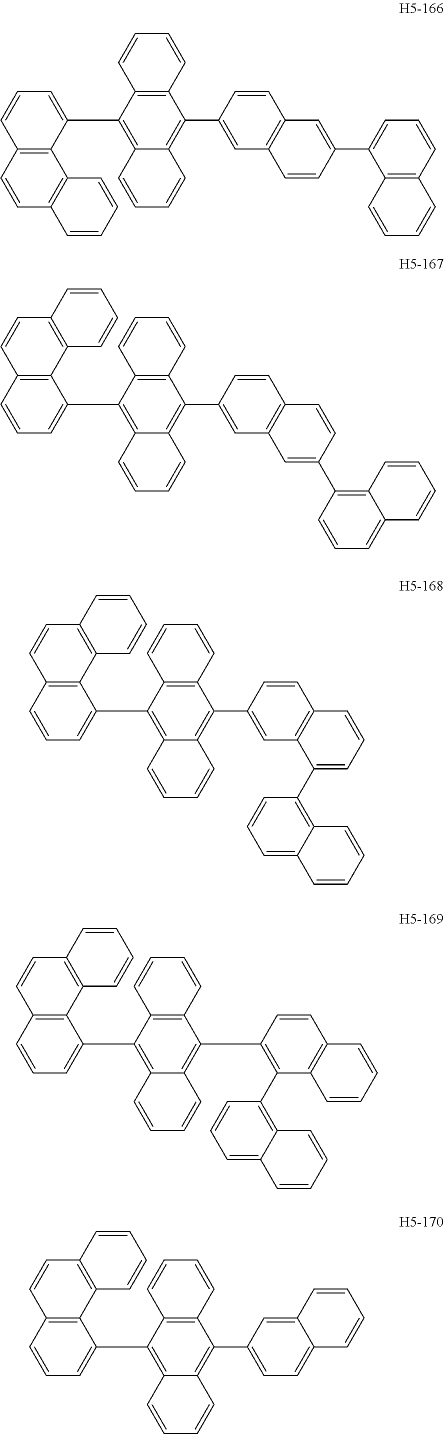 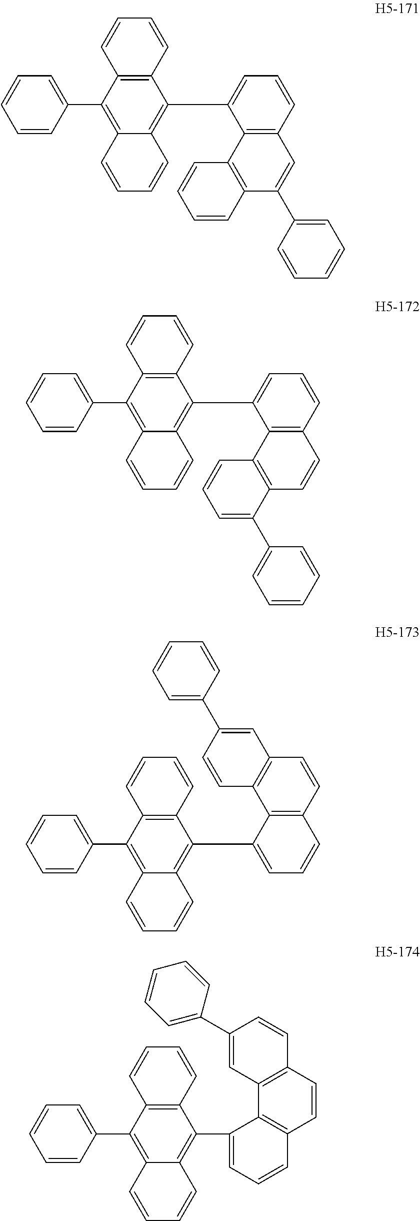 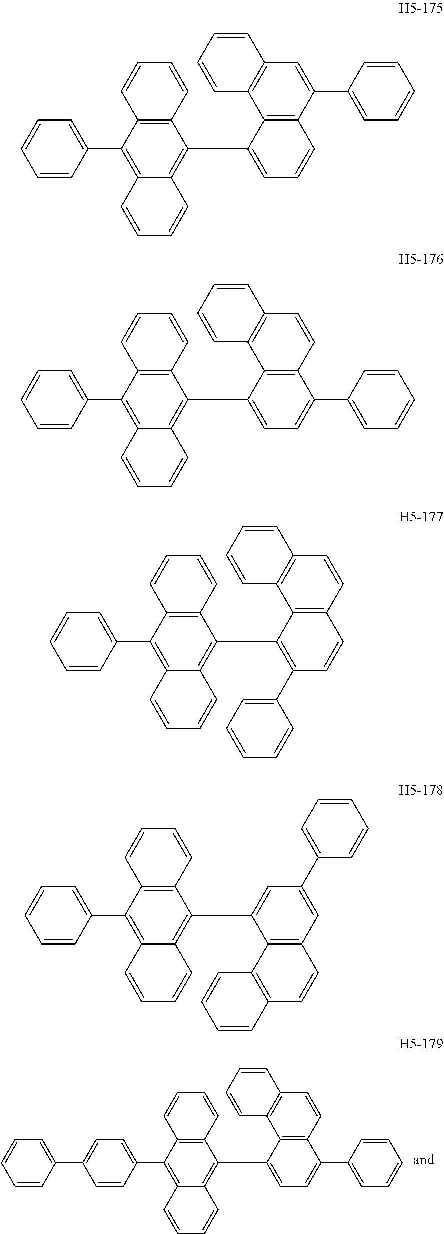 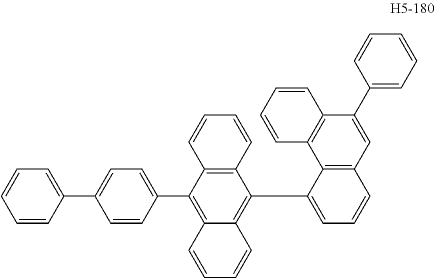 [SMILES: c1ccc(-c2ccc(-c3c4ccccc4c(-c4cccc5cc(-c6ccccc6)c6ccccc6c45)c4ccccc34)cc2)cc1]
In the compounds, Dₙ indicates that n hydrogen atoms are replaced with deuterium atoms, wherein n is an integer ranging from 1 to the maximum number of hydrogen atoms present in the compound.

13. The organic electroluminescent device according to claim 6, wherein the first light-emitting layer further comprises an additional host material, the second light-emitting layer further comprises an additional host material, or both the first light-emitting layer and the second light-emitting layer further comprise an additional host material.

14. The organic electroluminescent device according to claim 6, further comprising a third light-emitting layer adjacent to the second light-emitting layer.

15. The organic electroluminescent device according to claim 6, wherein both the first light-emitting layer and the second light-emitting layer are light-emitting layers that emit blue light.

16. An organic electroluminescent device comprising:
a first electrode;
a second electrode opposing the first electrode;
two or more light-emitting units disposed between the first electrode and the second electrode, and including at least one light-emitting layer; and
an n-type charge generation layer disposed between each of the light-emitting units,
wherein at least one of the light-emitting units comprises a first light-emitting layer and a second light-emitting layer, which are adjacent to each other,
wherein the n-type charge generation layer comprises at least one of compounds having a skeleton of phenanthroline, quinazoline, quinoxaline, terpyridine, and phenanthrooxazole.

17. The organic electroluminescent device according to claim 16, further comprising an electron transport layer positioned between the light-emitting unit and the second electrode, wherein the electron transport layer comprises an organic electroluminescent compound represented by the following Formula 3: 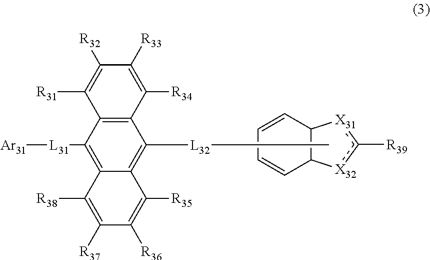
wherein,
R₃₁ to R₃₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀) aliphatic ring and a (C₆-C₃₀) aromatic ring;
R₃₉ represents hydrogen or a substituted or unsubstituted (C₁-C₃₀)alkyl;
L₃₁ and L₃₂ each independently represent a single bond or a substituted or unsubstituted (C₆-C₃₀)arylene;
Ar₃₁ represents a substituted or unsubstituted (C₆-C₃₀)aryl or a substituted or unsubstituted (3- to 30-membered)heteroaryl;
X₃₁ and X₃₂ each independently represent NR₄₀; and
R₄₀ represents a site linked to L₃₂, or hydrogen, deuterium, or a substituted or unsubstituted (C₁-C₃₀)alkyl.

18. The organic electroluminescent device according to claim 16, wherein the first light-emitting layer comprises a compound having a condensed ring comprising four or more rings.

19. The organic electroluminescent device according to claim 16, wherein at least one of the first light-emitting layer and the second light-emitting layer comprises a compound having at least one deuterium atom.

20. The organic electroluminescent device according to claim 16, wherein the first light-emitting layer comprises at least one compound having a skeleton of pyrene, benzanthracene, xanthene, chrysene, fluoranthene, triphenylene, benzoxanthene, dibenzochrysene, benzophenanthrene, and phenanthrofuran, and the second light-emitting layer comprises a compound having a skeleton of anthracene.

21. The organic electroluminescent device according to claim 16, wherein the n-type charge generation layer comprises at least one of the following compounds: 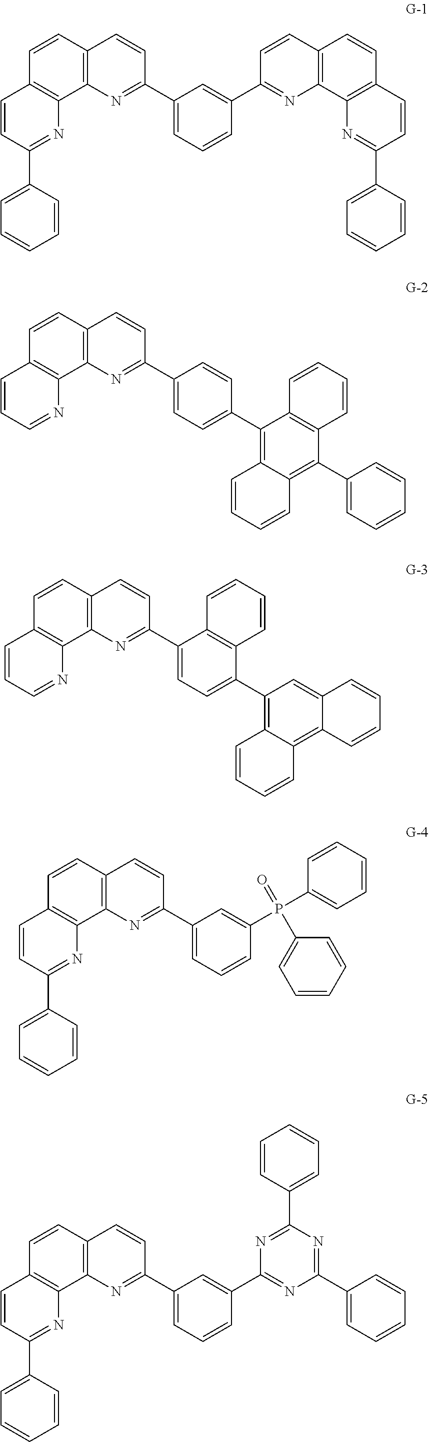 [SMILES: c1ccc(-c2ccc3ccc4ccc(-c5cccc(-c6ccc7ccc8ccc(-c9ccccc9)nc8c7n6)c5)nc4c3n2)cc1 ; c1ccc(-c2c3ccccc3c(-c3ccc(-c4ccc5ccc6cccnc6c5n4)cc3)c3ccccc23)cc1 ; c1ccc2c(c1)cc(-c1ccc(-c3ccc4ccc5cccnc5c4n3)c3ccccc13)c1ccccc12 ; O=P(c1ccccc1)(c1ccccc1)c1cccc(-c2ccc3ccc4ccc(-c5ccccc5)nc4c3n2)c1 ; c1ccc(-c2ccc3ccc4ccc(-c5cccc(-c6nc(-c7ccccc7)nc(-c7ccccc7)n6)c5)nc4c3n2)cc1] 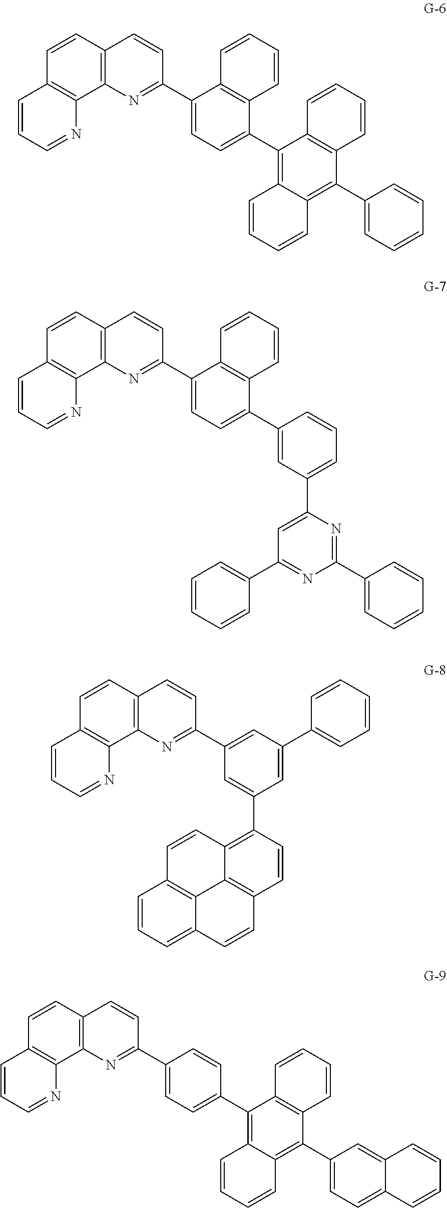 [SMILES: c1ccc(-c2c3ccccc3c(-c3ccc(-c4ccc5ccc6cccnc6c5n4)c4ccccc34)c3ccccc23)cc1 ; c1ccc(-c2cc(-c3cccc(-c4ccc(-c5ccc6ccc7cccnc7c6n5)c5ccccc45)c3)nc(-c3ccccc3)n2)cc1 ; c1ccc(-c2cc(-c3ccc4ccc5cccnc5c4n3)cc(-c3ccc4ccc5cccc6ccc3c4c56)c2)cc1 ; c1ccc2cc(-c3c4ccccc4c(-c4ccc(-c5ccc6ccc7cccnc7c6n5)cc4)c4ccccc34)ccc2c1] 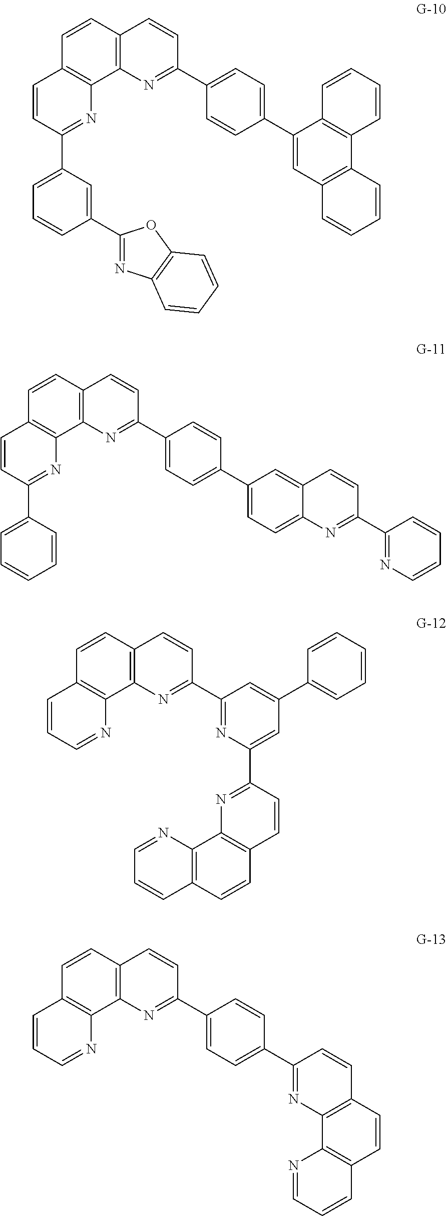 [SMILES: c1cc(-c2ccc3ccc4ccc(-c5ccc(-c6cc7ccccc7c7ccccc67)cc5)nc4c3n2)cc(-c2nc3ccccc3o2)c1 ; c1ccc(-c2ccc3ccc4ccc(-c5ccc(-c6ccc7nc(-c8ccccn8)ccc7c6)cc5)nc4c3n2)cc1 ; c1ccc(-c2cc(-c3ccc4ccc5cccnc5c4n3)nc(-c3ccc4ccc5cccnc5c4n3)c2)cc1 ; c1cnc2c(c1)ccc1ccc(-c3ccc(-c4ccc5ccc6cccnc6c5n4)cc3)nc12] 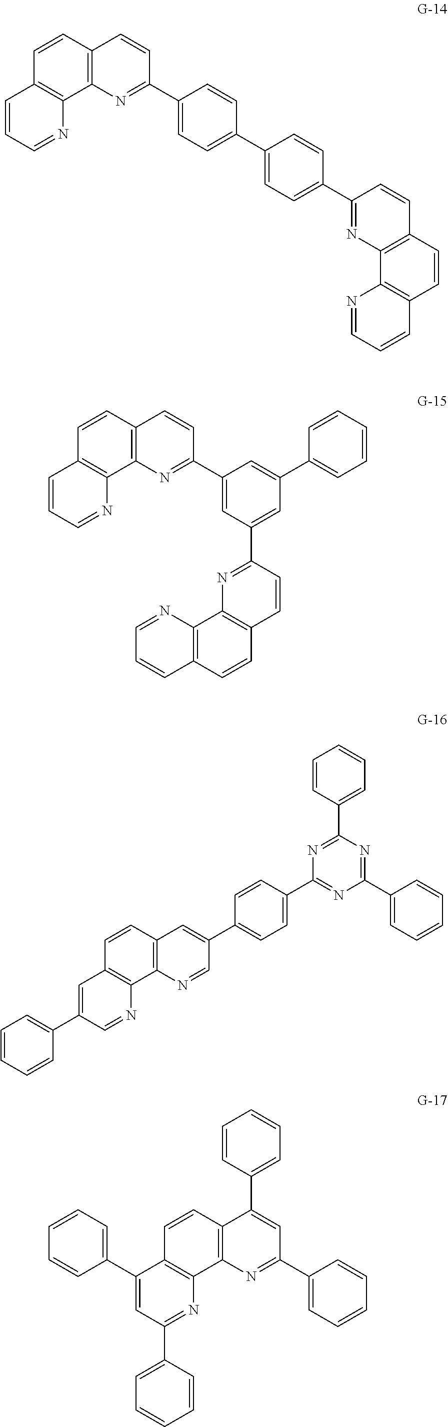 [SMILES: c1cnc2c(c1)ccc1ccc(-c3ccc(-c4ccc(-c5ccc6ccc7cccnc7c6n5)cc4)cc3)nc12 ; c1ccc(-c2cc(-c3ccc4ccc5cccnc5c4n3)cc(-c3ccc4ccc5cccnc5c4n3)c2)cc1 ; c1ccc(-c2cnc3c(ccc4cc(-c5ccc(-c6nc(-c7ccccc7)nc(-c7ccccc7)n6)cc5)cnc43)c2)cc1 ; c1ccc(-c2cc(-c3ccccc3)c3ccc4c(-c5ccccc5)cc(-c5ccccc5)nc4c3n2)cc1] 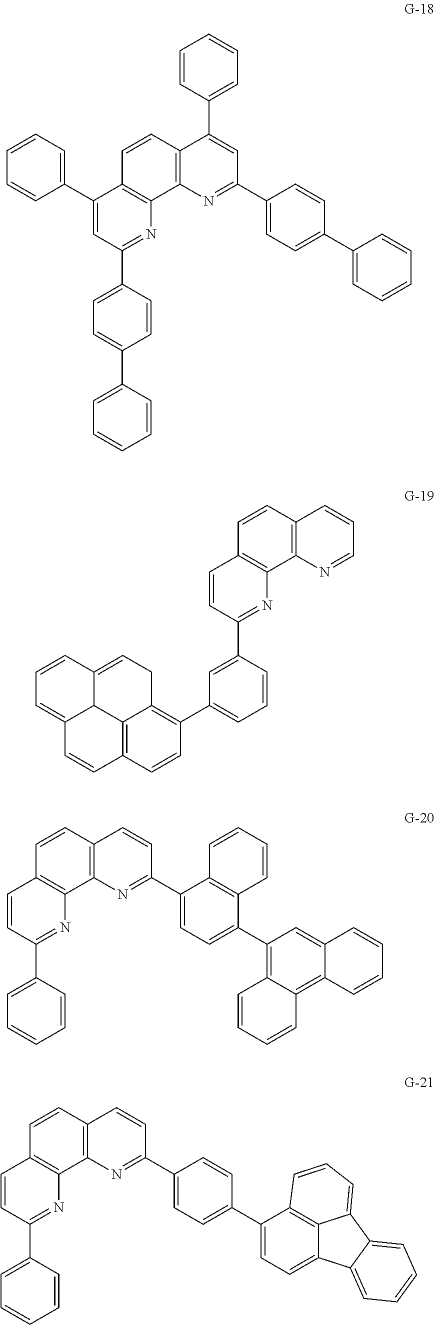 [SMILES: c1ccc(-c2ccc(-c3cc(-c4ccccc4)c4ccc5c(-c6ccccc6)cc(-c6ccc(-c7ccccc7)cc6)nc5c4n3)cc2)cc1 ; C1=CC2=CCc3c(-c4cccc(-c5ccc6ccc7cccnc7c6n5)c4)ccc4c3C2C(=C1)C=C4 ; c1ccc(-c2ccc3ccc4ccc(-c5ccc(-c6cc7ccccc7c7ccccc67)c6ccccc56)nc4c3n2)cc1 ; c1ccc(-c2ccc3ccc4ccc(-c5ccc(-c6ccc7c8c(cccc68)-c6ccccc6-7)cc5)nc4c3n2)cc1] 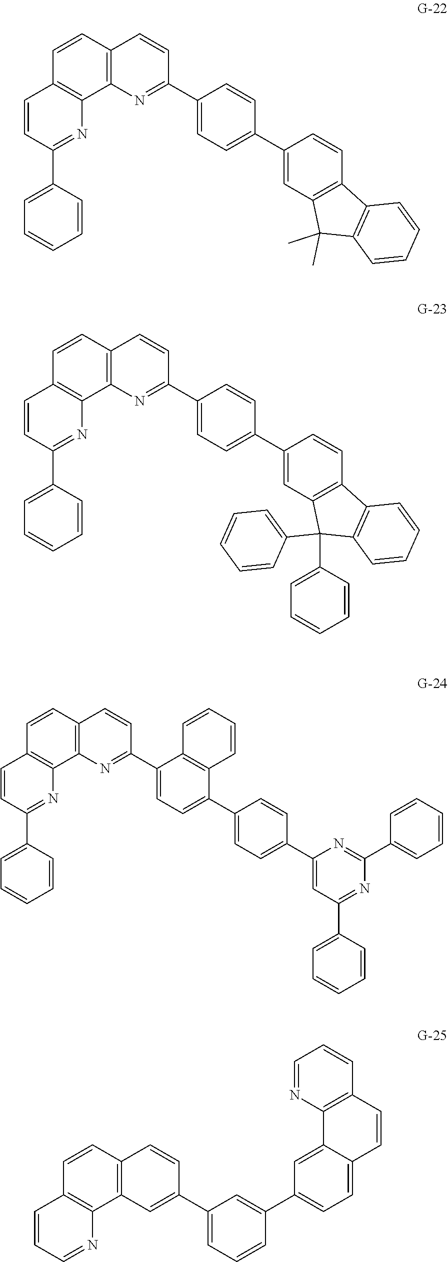 [SMILES: CC1(C)c2ccccc2-c2ccc(-c3ccc(-c4ccc5ccc6ccc(-c7ccccc7)nc6c5n4)cc3)cc21 ; c1ccc(-c2ccc3ccc4ccc(-c5ccc(-c6ccc7c(c6)C(c6ccccc6)(c6ccccc6)c6ccccc6-7)cc5)nc4c3n2)cc1 ; c1ccc(-c2cc(-c3ccc(-c4ccc(-c5ccc6ccc7ccc(-c8ccccc8)nc7c6n5)c5ccccc45)cc3)nc(-c3ccccc3)n2)cc1 ; c1cc(-c2ccc3ccc4cccnc4c3c2)cc(-c2ccc3ccc4cccnc4c3c2)c1] 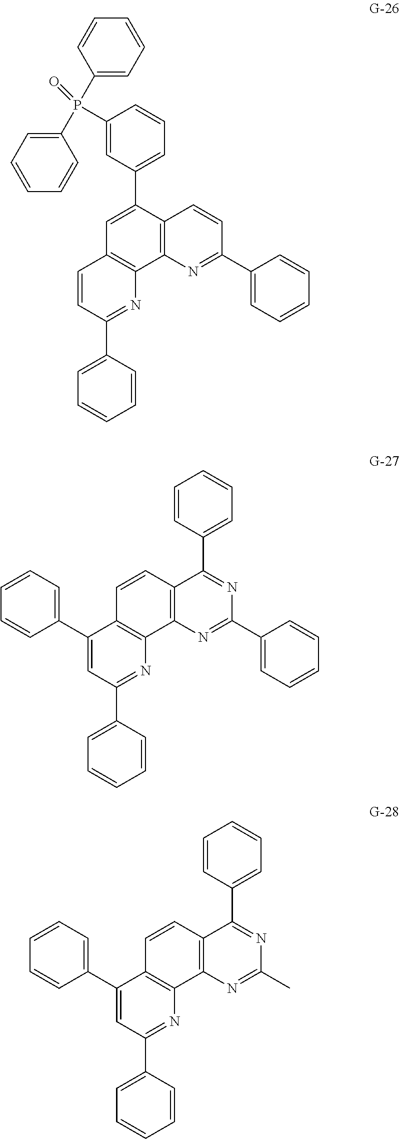 [SMILES: O=P(c1ccccc1)(c1ccccc1)c1cccc(-c2cc3ccc(-c4ccccc4)nc3c3nc(-c4ccccc4)ccc23)c1 ; c1ccc(-c2cc(-c3ccccc3)c3ccc4c(-c5ccccc5)nc(-c5ccccc5)nc4c3n2)cc1 ; Cc1nc(-c2ccccc2)c2ccc3c(-c4ccccc4)cc(-c4ccccc4)nc3c2n1] 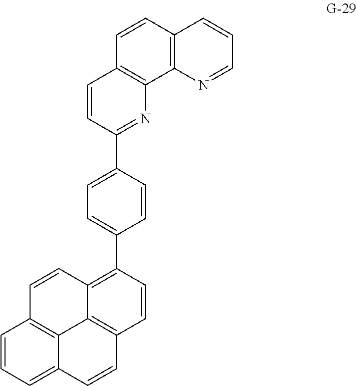 [SMILES: c1cnc2c(c1)ccc1ccc(-c3ccc(-c4ccc5ccc6cccc7ccc4c5c67)cc3)nc12] 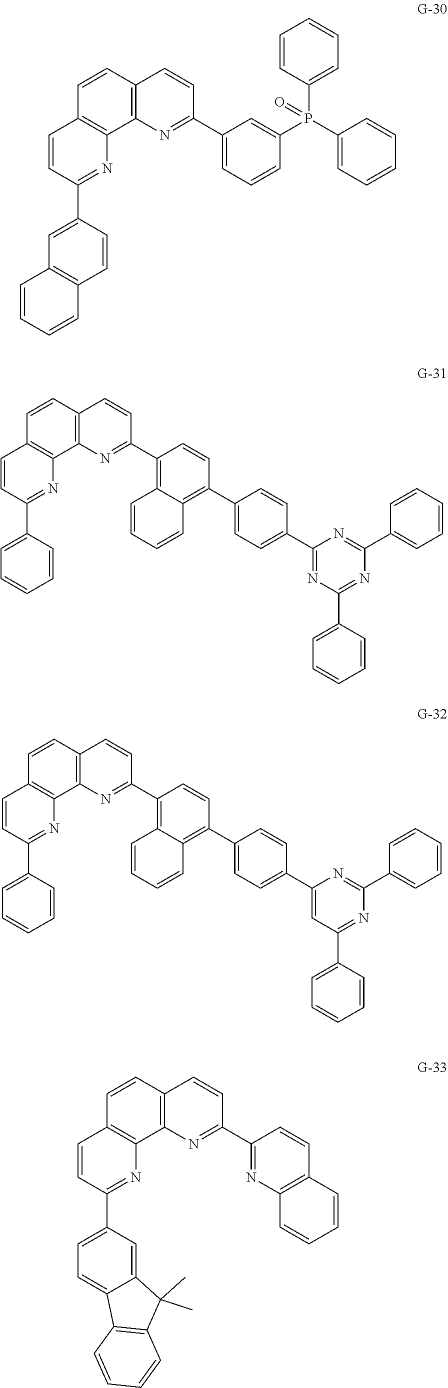 [SMILES: O=P(c1ccccc1)(c1ccccc1)c1cccc(-c2ccc3ccc4ccc(-c5ccc6ccccc6c5)nc4c3n2)c1 ; c1ccc(-c2ccc3ccc4ccc(-c5ccc(-c6ccc(-c7nc(-c8ccccc8)nc(-c8ccccc8)n7)cc6)c6ccccc56)nc4c3n2)cc1 ; c1ccc(-c2cc(-c3ccc(-c4ccc(-c5ccc6ccc7ccc(-c8ccccc8)nc7c6n5)c5ccccc45)cc3)nc(-c3ccccc3)n2)cc1 ; CC1(C)c2ccccc2-c2ccc(-c3ccc4ccc5ccc(-c6ccc7ccccc7n6)nc5c4n3)cc21] 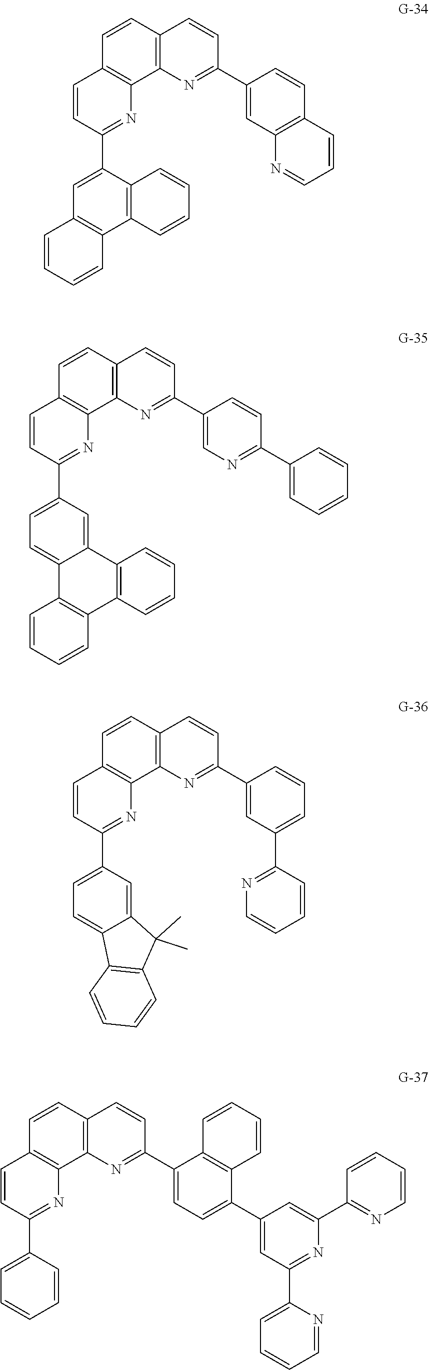 [SMILES: c1cnc2cc(-c3ccc4ccc5ccc(-c6cc7ccccc7c7ccccc67)nc5c4n3)ccc2c1 ; c1ccc(-c2ccc(-c3ccc4ccc5ccc(-c6ccc7c8ccccc8c8ccccc8c7c6)nc5c4n3)cn2)cc1 ; CC1(C)c2ccccc2-c2ccc(-c3ccc4ccc5ccc(-c6cccc(-c7ccccn7)c6)nc5c4n3)cc21 ; c1ccc(-c2ccc3ccc4ccc(-c5ccc(-c6cc(-c7ccccn7)nc(-c7ccccn7)c6)c6ccccc56)nc4c3n2)cc1] 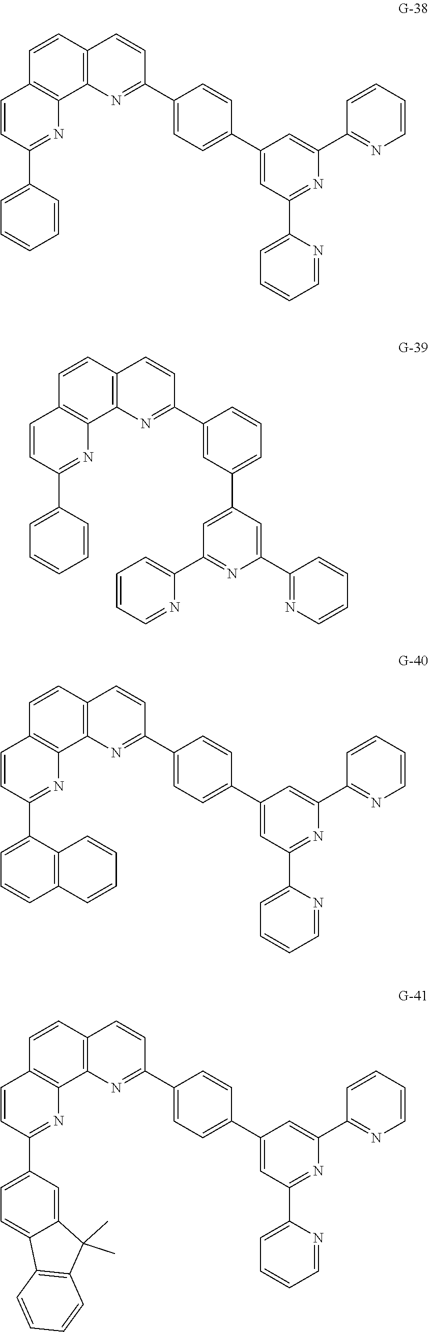 [SMILES: c1ccc(-c2ccc3ccc4ccc(-c5ccc(-c6cc(-c7ccccn7)nc(-c7ccccn7)c6)cc5)nc4c3n2)cc1 ; c1ccc(-c2ccc3ccc4ccc(-c5cccc(-c6cc(-c7ccccn7)nc(-c7ccccn7)c6)c5)nc4c3n2)cc1 ; c1ccc(-c2cc(-c3ccc(-c4ccc5ccc6ccc(-c7cccc8ccccc78)nc6c5n4)cc3)cc(-c3ccccn3)n2)nc1 ; CC1(C)c2ccccc2-c2ccc(-c3ccc4ccc5ccc(-c6ccc(-c7cc(-c8ccccn8)nc(-c8ccccn8)c7)cc6)nc5c4n3)cc21] 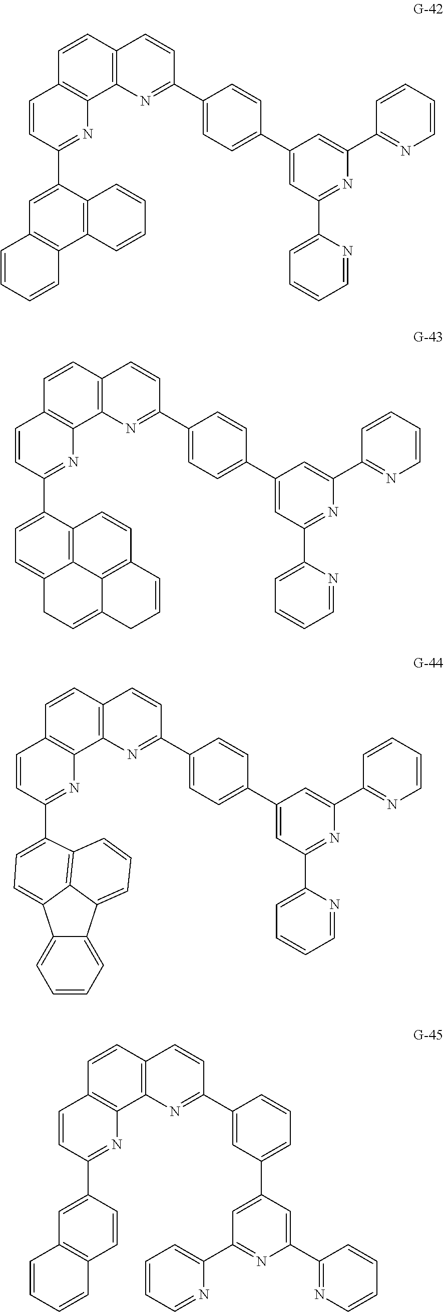 [SMILES: c1ccc(-c2cc(-c3ccc(-c4ccc5ccc6ccc(-c7cc8ccccc8c8ccccc78)nc6c5n4)cc3)cc(-c3ccccn3)n2)nc1 ; C1=Cc2ccc3c(-c4ccc5ccc6ccc(-c7ccc(-c8cc(-c9ccccn9)nc(-c9ccccn9)c8)cc7)nc6c5n4)ccc4c3c2C(=CC4)C1 ; c1ccc(-c2cc(-c3ccc(-c4ccc5ccc6ccc(-c7ccc8c9c(cccc79)-c7ccccc7-8)nc6c5n4)cc3)cc(-c3ccccn3)n2)nc1 ; c1ccc(-c2cc(-c3cccc(-c4ccc5ccc6ccc(-c7ccc8ccccc8c7)nc6c5n4)c3)cc(-c3ccccn3)n2)nc1] 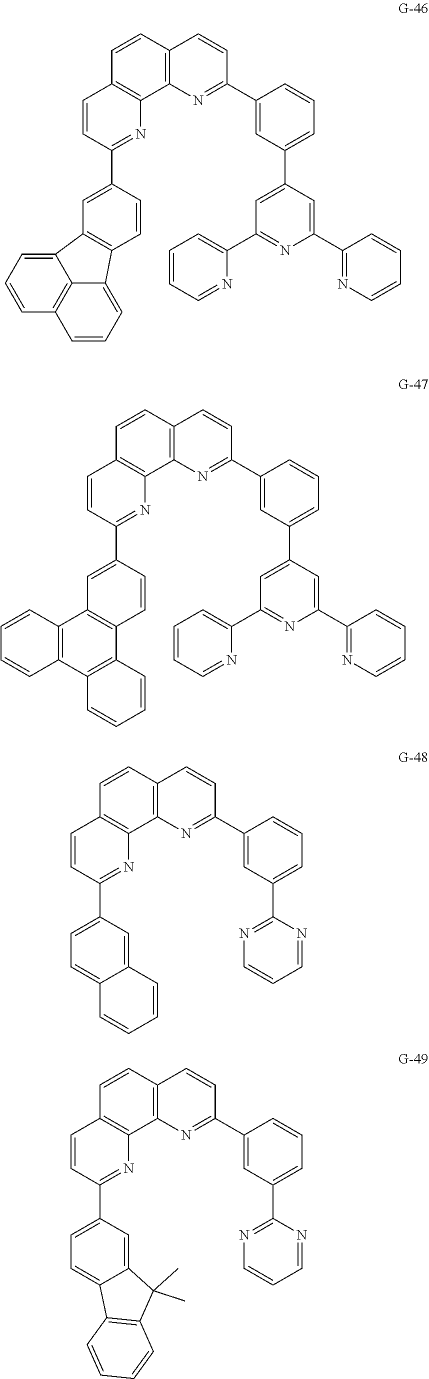 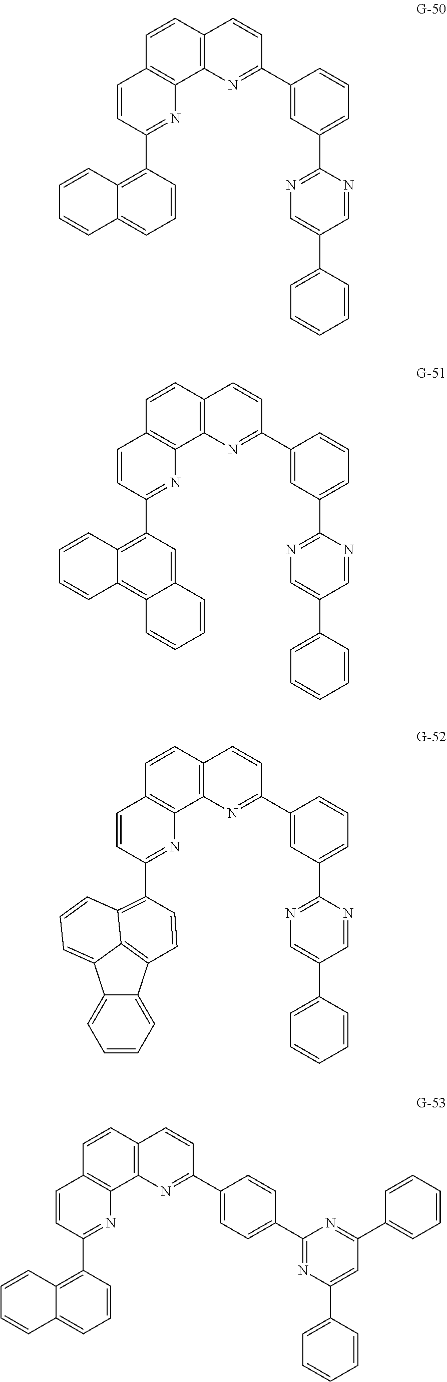 [SMILES: c1ccc(-c2cnc(-c3cccc(-c4ccc5ccc6ccc(-c7cccc8ccccc78)nc6c5n4)c3)nc2)cc1 ; c1ccc(-c2cnc(-c3cccc(-c4ccc5ccc6ccc(-c7cc8ccccc8c8ccccc78)nc6c5n4)c3)nc2)cc1 ; c1ccc(-c2cnc(-c3cccc(-c4ccc5ccc6ccc(-c7ccc8c9c(cccc79)-c7ccccc7-8)nc6c5n4)c3)nc2)cc1 ; c1ccc(-c2cc(-c3ccccc3)nc(-c3ccc(-c4ccc5ccc6ccc(-c7cccc8ccccc78)nc6c5n4)cc3)n2)cc1] 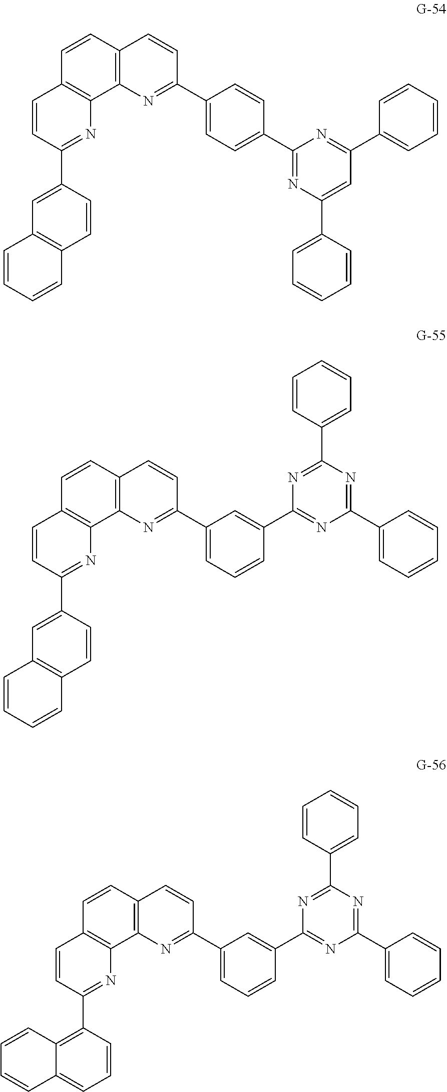 [SMILES: c1ccc(-c2cc(-c3ccccc3)nc(-c3ccc(-c4ccc5ccc6ccc(-c7ccc8ccccc8c7)nc6c5n4)cc3)n2)cc1 ; c1ccc(-c2nc(-c3ccccc3)nc(-c3cccc(-c4ccc5ccc6ccc(-c7ccc8ccccc8c7)nc6c5n4)c3)n2)cc1 ; c1ccc(-c2nc(-c3ccccc3)nc(-c3cccc(-c4ccc5ccc6ccc(-c7cccc8ccccc78)nc6c5n4)c3)n2)cc1] 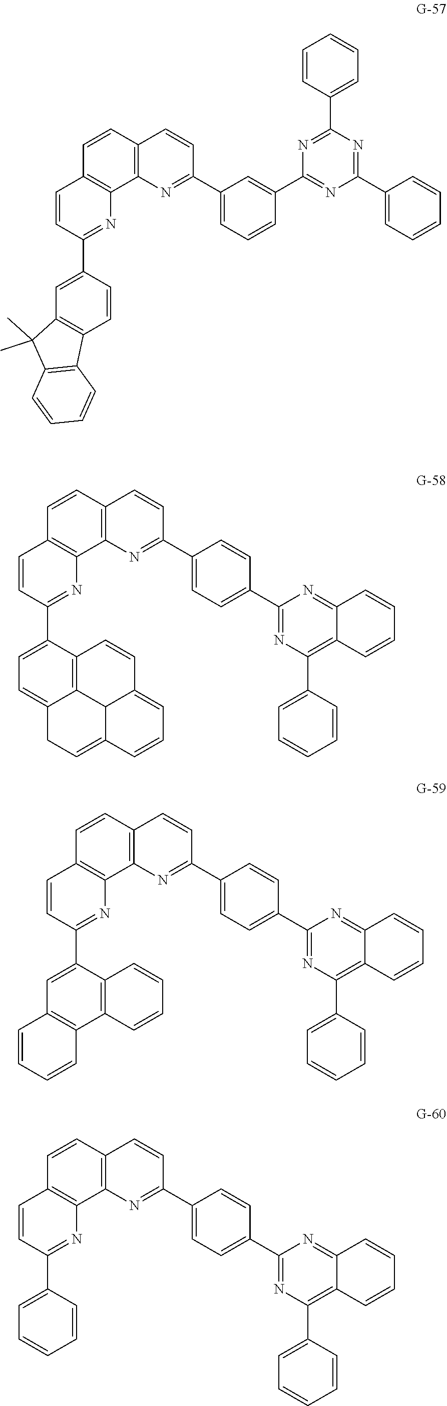 [SMILES: CC1(C)c2ccccc2-c2ccc(-c3ccc4ccc5ccc(-c6cccc(-c7nc(-c8ccccc8)nc(-c8ccccc8)n7)c6)nc5c4n3)cc21 ; C1=CC2=CCc3ccc(-c4ccc5ccc6ccc(-c7ccc(-c8nc(-c9ccccc9)c9ccccc9n8)cc7)nc6c5n4)c4c3C2C(=C1)C=C4 ; c1ccc(-c2nc(-c3ccc(-c4ccc5ccc6ccc(-c7cc8ccccc8c8ccccc78)nc6c5n4)cc3)nc3ccccc23)cc1 ; c1ccc(-c2ccc3ccc4ccc(-c5ccc(-c6nc(-c7ccccc7)c7ccccc7n6)cc5)nc4c3n2)cc1] 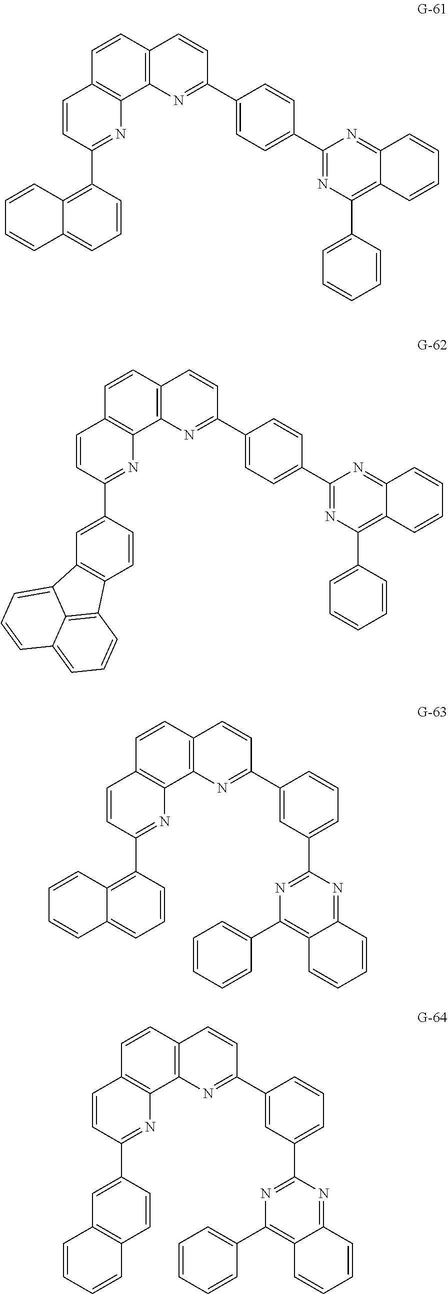 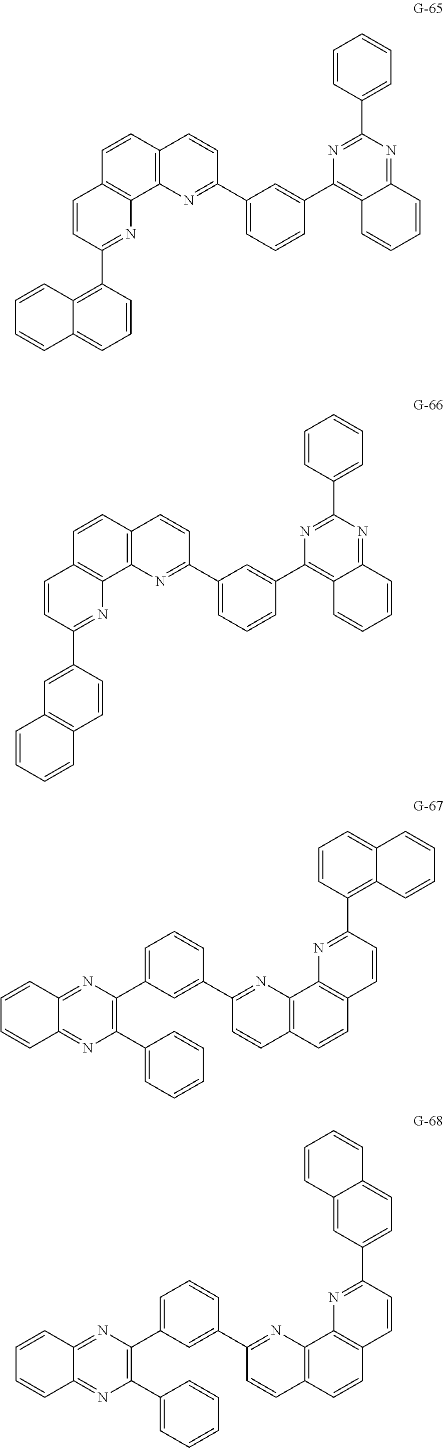 [SMILES: c1ccc(-c2nc(-c3cccc(-c4ccc5ccc6ccc(-c7cccc8ccccc78)nc6c5n4)c3)c3ccccc3n2)cc1 ; c1ccc(-c2nc(-c3cccc(-c4ccc5ccc6ccc(-c7ccc8ccccc8c7)nc6c5n4)c3)c3ccccc3n2)cc1 ; c1ccc(-c2nc3ccccc3nc2-c2cccc(-c3ccc4ccc5ccc(-c6cccc7ccccc67)nc5c4n3)c2)cc1 ; c1ccc(-c2nc3ccccc3nc2-c2cccc(-c3ccc4ccc5ccc(-c6ccc7ccccc7c6)nc5c4n3)c2)cc1] 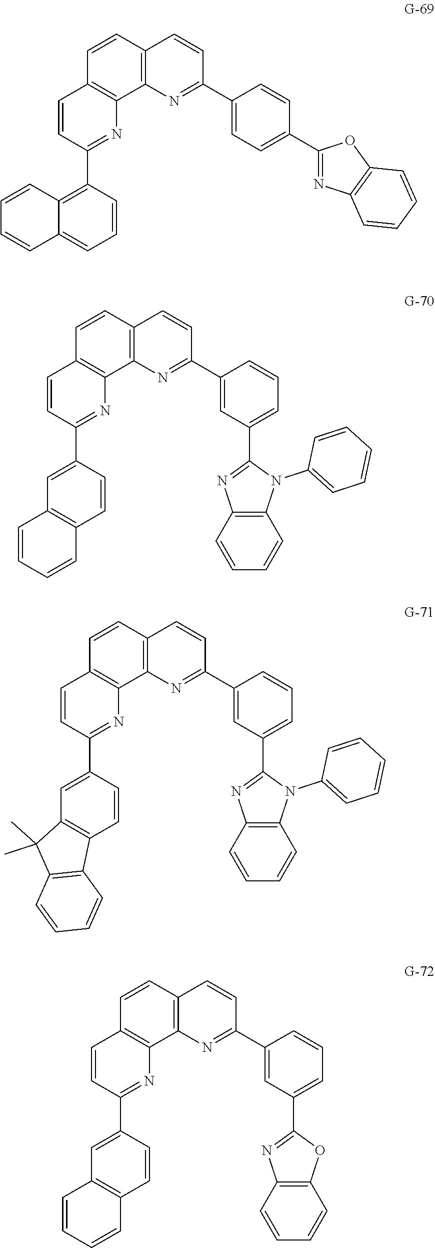 [SMILES: c1ccc2c(-c3ccc4ccc5ccc(-c6ccc(-c7nc8ccccc8o7)cc6)nc5c4n3)cccc2c1 ; c1ccc(-n2c(-c3cccc(-c4ccc5ccc6ccc(-c7ccc8ccccc8c7)nc6c5n4)c3)nc3ccccc32)cc1 ; CC1(C)c2ccccc2-c2ccc(-c3ccc4ccc5ccc(-c6cccc(-c7nc8ccccc8n7-c7ccccc7)c6)nc5c4n3)cc21 ; c1cc(-c2ccc3ccc4ccc(-c5ccc6ccccc6c5)nc4c3n2)cc(-c2nc3ccccc3o2)c1] 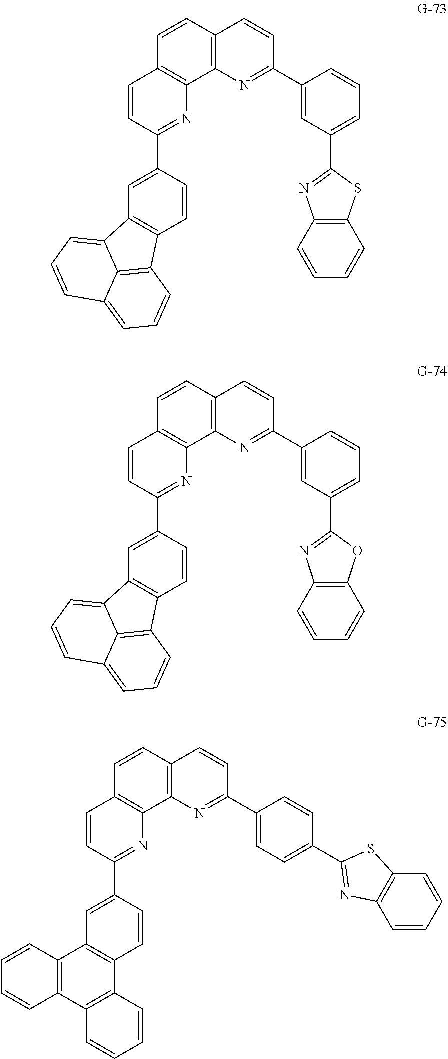 [SMILES: c1cc(-c2ccc3ccc4ccc(-c5ccc6c(c5)-c5cccc7cccc-6c57)nc4c3n2)cc(-c2nc3ccccc3s2)c1 ; c1cc(-c2ccc3ccc4ccc(-c5ccc6c(c5)-c5cccc7cccc-6c57)nc4c3n2)cc(-c2nc3ccccc3o2)c1 ; c1ccc2sc(-c3ccc(-c4ccc5ccc6ccc(-c7ccc8c9ccccc9c9ccccc9c8c7)nc6c5n4)cc3)nc2c1] 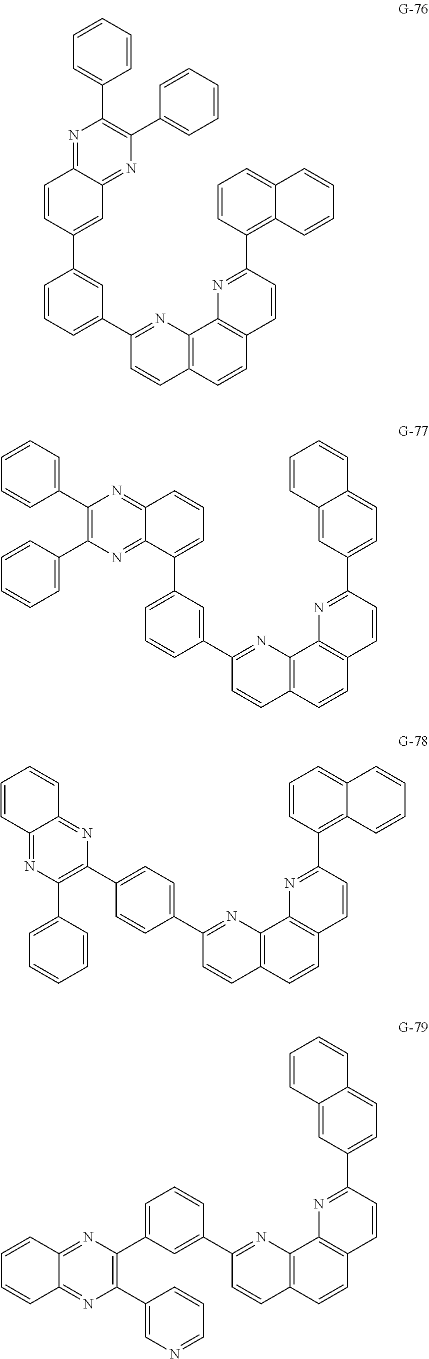 [SMILES: c1ccc(-c2nc3ccc(-c4cccc(-c5ccc6ccc7ccc(-c8cccc9ccccc89)nc7c6n5)c4)cc3nc2-c2ccccc2)cc1 ; c1ccc(-c2nc3cccc(-c4cccc(-c5ccc6ccc7ccc(-c8ccc9ccccc9c8)nc7c6n5)c4)c3nc2-c2ccccc2)cc1 ; c1ccc(-c2nc3ccccc3nc2-c2ccc(-c3ccc4ccc5ccc(-c6cccc7ccccc67)nc5c4n3)cc2)cc1 ; c1cncc(-c2nc3ccccc3nc2-c2cccc(-c3ccc4ccc5ccc(-c6ccc7ccccc7c6)nc5c4n3)c2)c1] 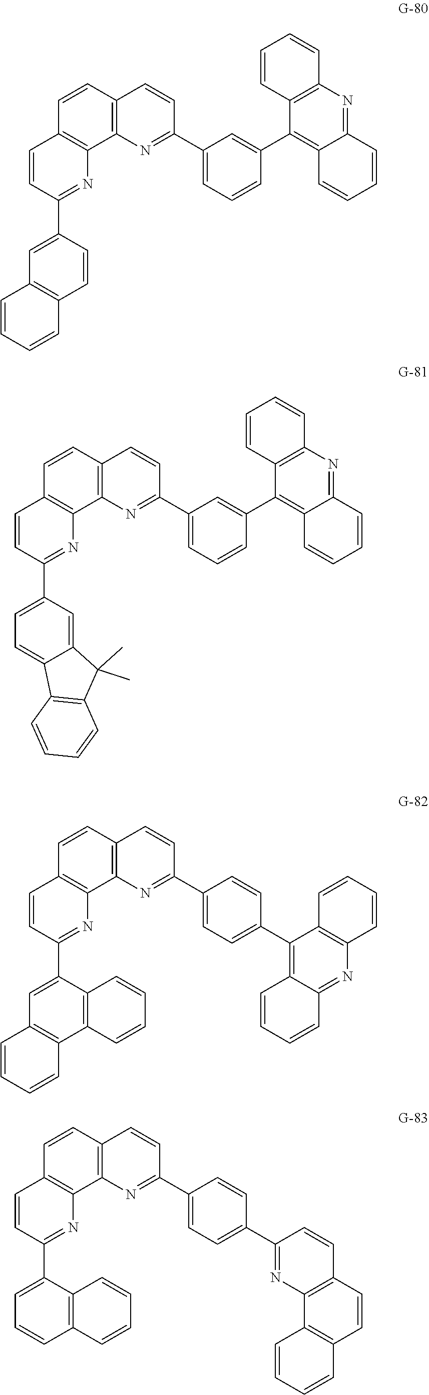 [SMILES: c1cc(-c2ccc3ccc4ccc(-c5ccc6ccccc6c5)nc4c3n2)cc(-c2c3ccccc3nc3ccccc23)c1 ; CC1(C)c2ccccc2-c2ccc(-c3ccc4ccc5ccc(-c6cccc(-c7c8ccccc8nc8ccccc78)c6)nc5c4n3)cc21 ; c1ccc2c(c1)cc(-c1ccc3ccc4ccc(-c5ccc(-c6c7ccccc7nc7ccccc67)cc5)nc4c3n1)c1ccccc12 ; c1ccc2c(-c3ccc4ccc5ccc(-c6ccc(-c7ccc8ccc9ccccc9c8n7)cc6)nc5c4n3)cccc2c1] 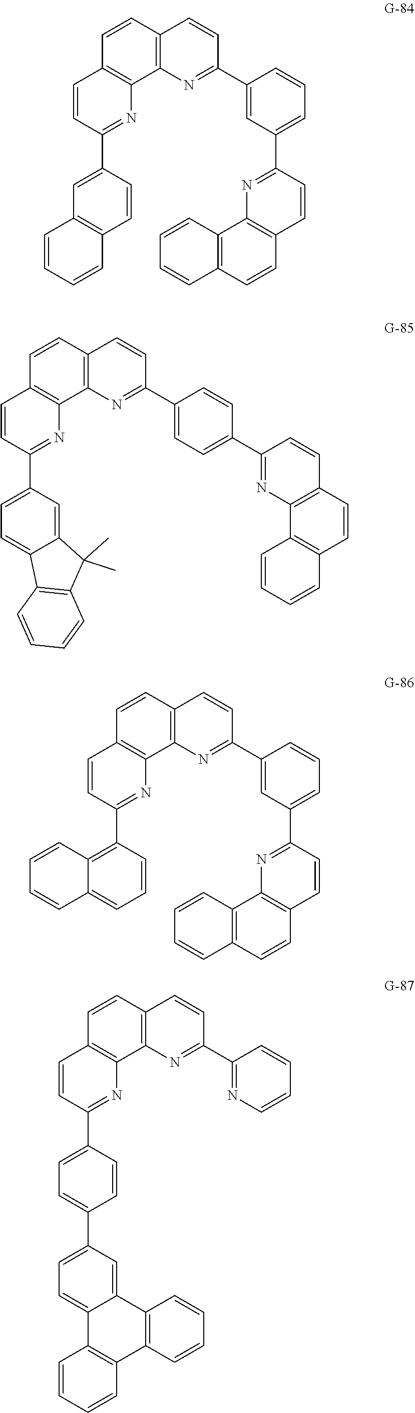 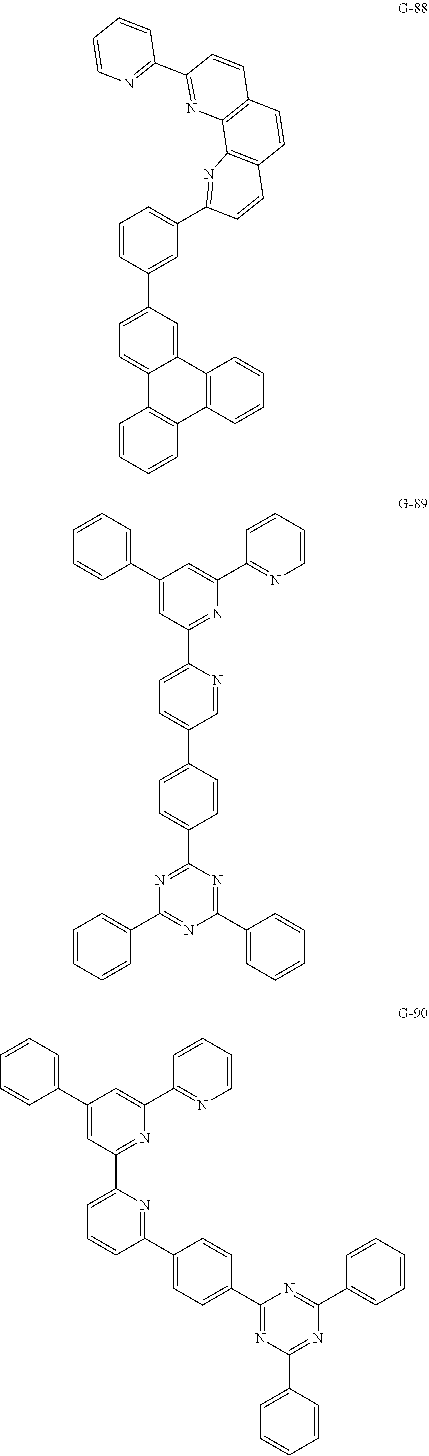 [SMILES: c1ccc(-c2ccc3ccc4ccc(-c5cccc(-c6ccc7c8ccccc8c8ccccc8c7c6)c5)nc4c3n2)nc1 ; c1ccc(-c2cc(-c3ccccn3)nc(-c3ccc(-c4ccc(-c5nc(-c6ccccc6)nc(-c6ccccc6)n5)cc4)cn3)c2)cc1 ; c1ccc(-c2cc(-c3ccccn3)nc(-c3cccc(-c4ccc(-c5nc(-c6ccccc6)nc(-c6ccccc6)n5)cc4)n3)c2)cc1] 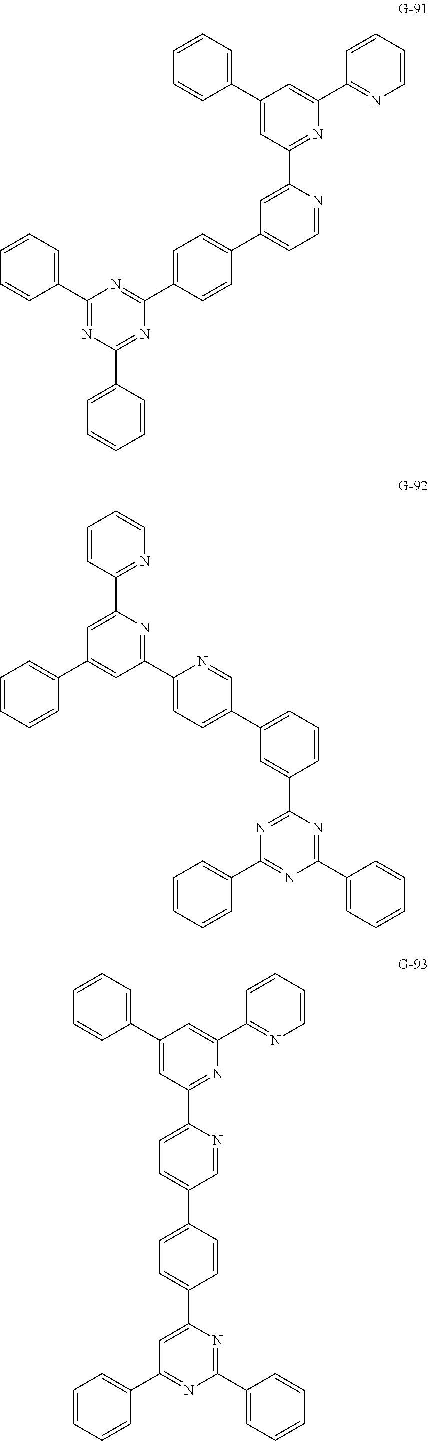 [SMILES: c1ccc(-c2cc(-c3ccccn3)nc(-c3cc(-c4ccc(-c5nc(-c6ccccc6)nc(-c6ccccc6)n5)cc4)ccn3)c2)cc1 ; c1ccc(-c2cc(-c3ccccn3)nc(-c3ccc(-c4cccc(-c5nc(-c6ccccc6)nc(-c6ccccc6)n5)c4)cn3)c2)cc1 ; c1ccc(-c2cc(-c3ccccn3)nc(-c3ccc(-c4ccc(-c5cc(-c6ccccc6)nc(-c6ccccc6)n5)cc4)cn3)c2)cc1] 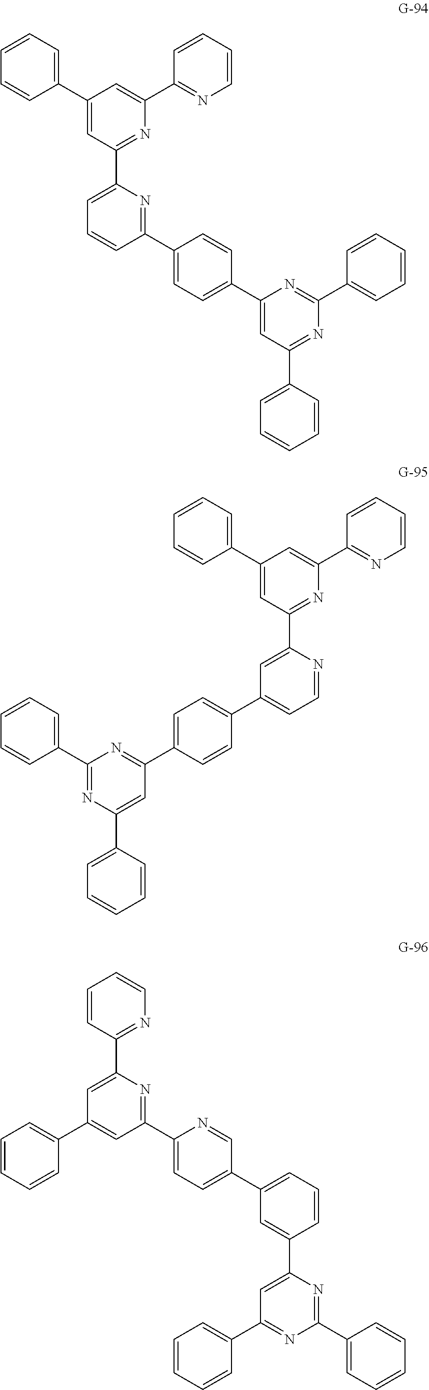 [SMILES: c1ccc(-c2cc(-c3ccccn3)nc(-c3cccc(-c4ccc(-c5cc(-c6ccccc6)nc(-c6ccccc6)n5)cc4)n3)c2)cc1 ; c1ccc(-c2cc(-c3ccccn3)nc(-c3cc(-c4ccc(-c5cc(-c6ccccc6)nc(-c6ccccc6)n5)cc4)ccn3)c2)cc1 ; c1ccc(-c2cc(-c3ccccn3)nc(-c3ccc(-c4cccc(-c5cc(-c6ccccc6)nc(-c6ccccc6)n5)c4)cn3)c2)cc1] 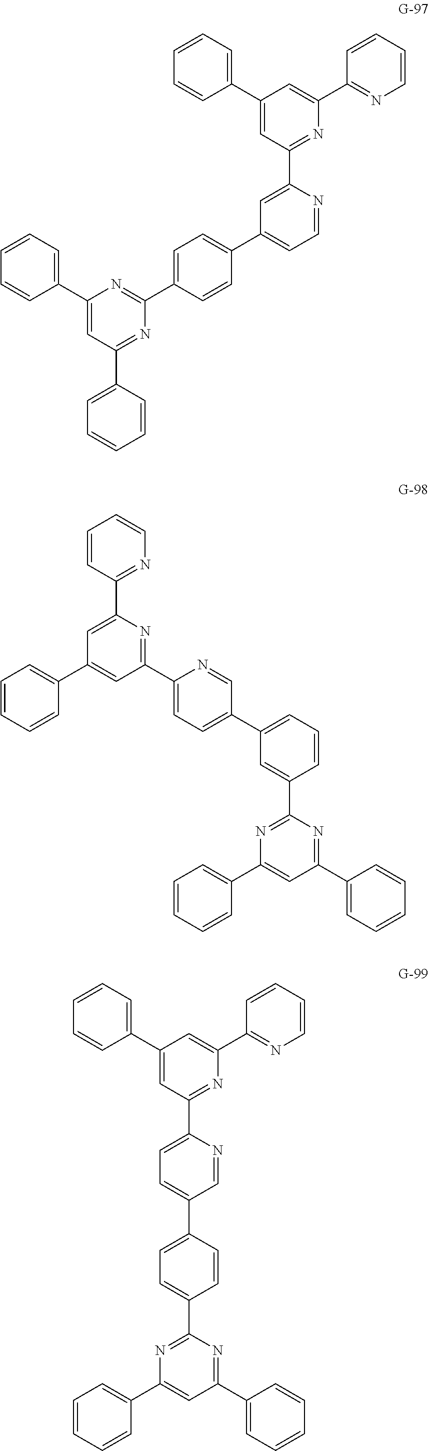 [SMILES: c1ccc(-c2cc(-c3ccccn3)nc(-c3cc(-c4ccc(-c5nc(-c6ccccc6)cc(-c6ccccc6)n5)cc4)ccn3)c2)cc1 ; c1ccc(-c2cc(-c3ccccn3)nc(-c3ccc(-c4cccc(-c5nc(-c6ccccc6)cc(-c6ccccc6)n5)c4)cn3)c2)cc1 ; c1ccc(-c2cc(-c3ccccn3)nc(-c3ccc(-c4ccc(-c5nc(-c6ccccc6)cc(-c6ccccc6)n5)cc4)cn3)c2)cc1] 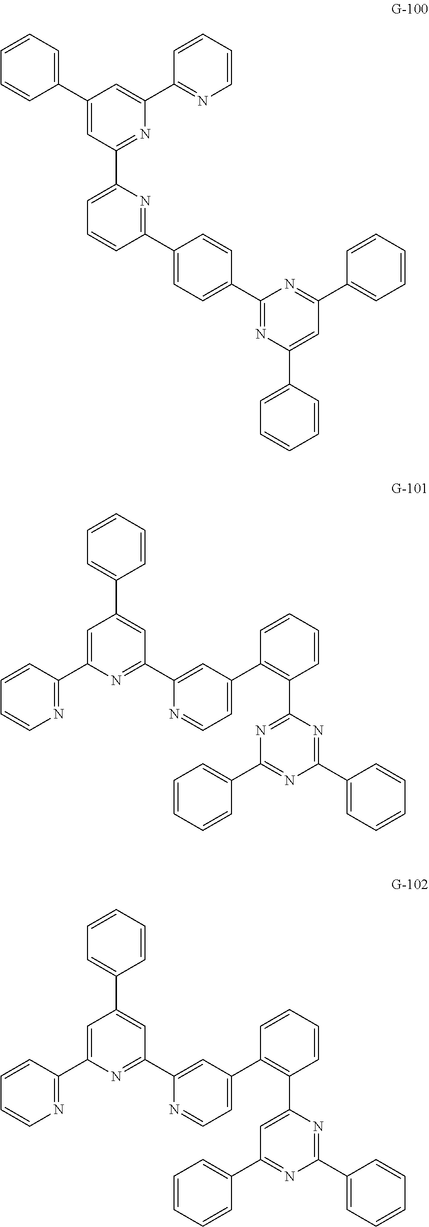 [SMILES: c1ccc(-c2cc(-c3ccccn3)nc(-c3cccc(-c4ccc(-c5nc(-c6ccccc6)cc(-c6ccccc6)n5)cc4)n3)c2)cc1 ; c1ccc(-c2cc(-c3ccccn3)nc(-c3cc(-c4ccccc4-c4nc(-c5ccccc5)nc(-c5ccccc5)n4)ccn3)c2)cc1 ; c1ccc(-c2cc(-c3ccccn3)nc(-c3cc(-c4ccccc4-c4cc(-c5ccccc5)nc(-c5ccccc5)n4)ccn3)c2)cc1] 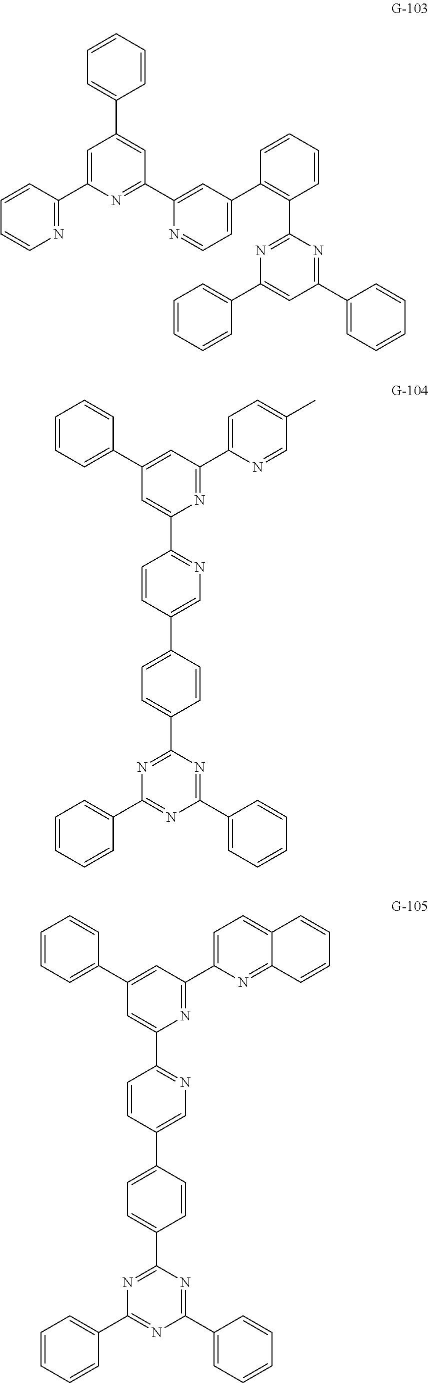 [SMILES: c1ccc(-c2cc(-c3ccccn3)nc(-c3cc(-c4ccccc4-c4nc(-c5ccccc5)cc(-c5ccccc5)n4)ccn3)c2)cc1 ; Cc1ccc(-c2cc(-c3ccccc3)cc(-c3ccc(-c4ccc(-c5nc(-c6ccccc6)nc(-c6ccccc6)n5)cc4)cn3)n2)nc1 ; c1ccc(-c2cc(-c3ccc(-c4ccc(-c5nc(-c6ccccc6)nc(-c6ccccc6)n5)cc4)cn3)nc(-c3ccc4ccccc4n3)c2)cc1] 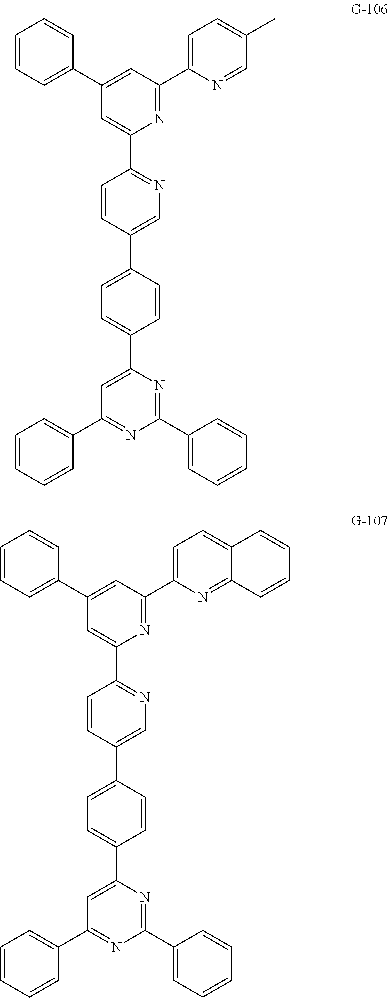 [SMILES: Cc1ccc(-c2cc(-c3ccccc3)cc(-c3ccc(-c4ccc(-c5cc(-c6ccccc6)nc(-c6ccccc6)n5)cc4)cn3)n2)nc1 ; c1ccc(-c2cc(-c3ccc(-c4ccc(-c5cc(-c6ccccc6)nc(-c6ccccc6)n5)cc4)cn3)nc(-c3ccc4ccccc4n3)c2)cc1] 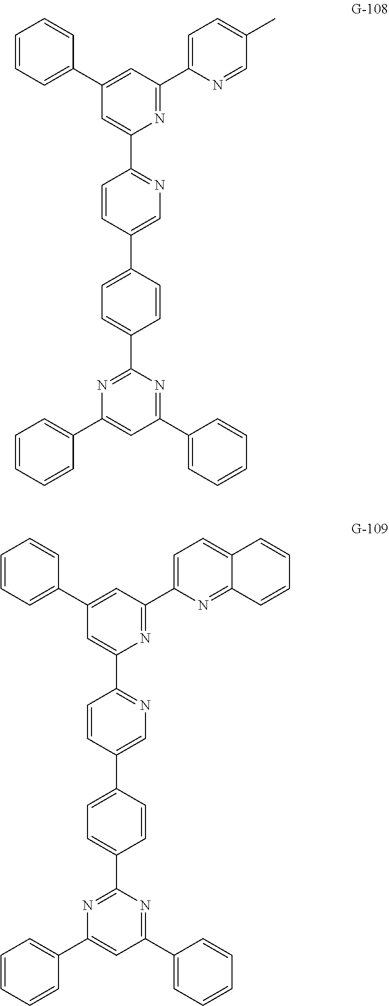 [SMILES: Cc1ccc(-c2cc(-c3ccccc3)cc(-c3ccc(-c4ccc(-c5nc(-c6ccccc6)cc(-c6ccccc6)n5)cc4)cn3)n2)nc1 ; c1ccc(-c2cc(-c3ccc(-c4ccc(-c5nc(-c6ccccc6)cc(-c6ccccc6)n5)cc4)cn3)nc(-c3ccc4ccccc4n3)c2)cc1] 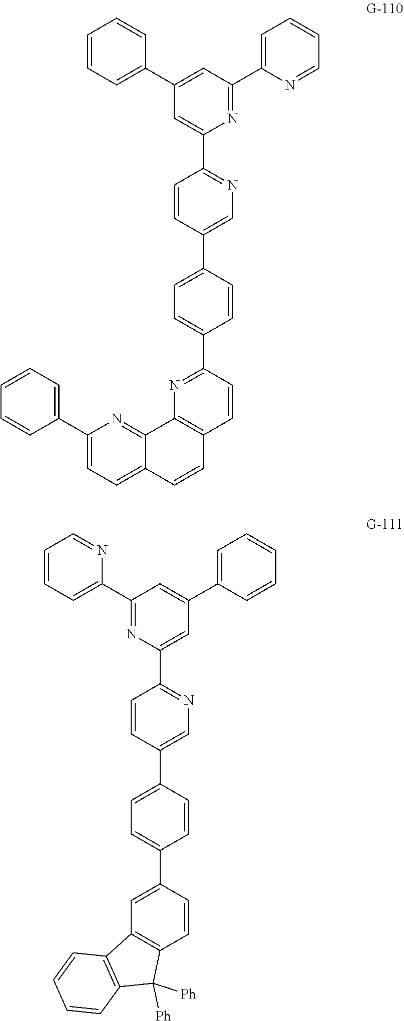 [SMILES: c1ccc(-c2cc(-c3ccccn3)nc(-c3ccc(-c4ccc(-c5ccc6ccc7ccc(-c8ccccc8)nc7c6n5)cc4)cn3)c2)cc1 ; c1ccc(-c2cc(-c3ccccn3)nc(-c3ccc(-c4ccc(-c5ccc6c(c5)-c5ccccc5C6(c5ccccc5)c5ccccc5)cc4)cn3)c2)cc1] 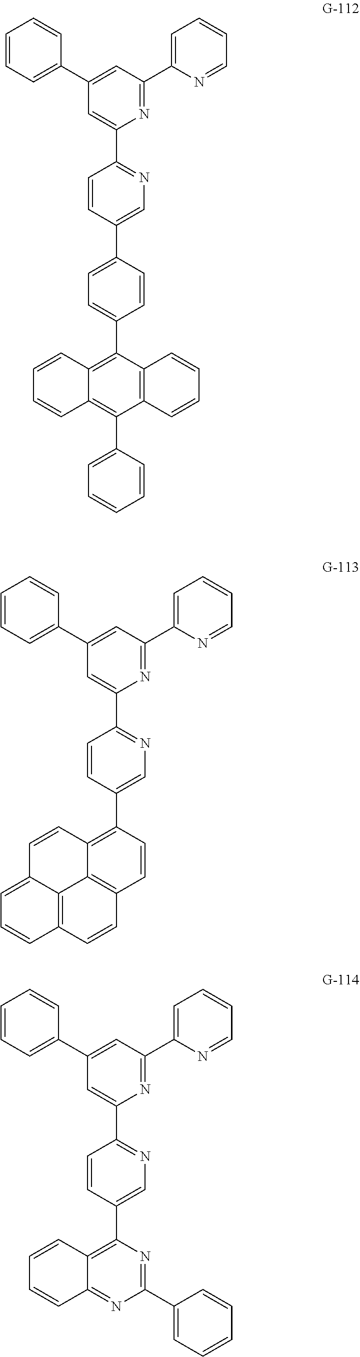 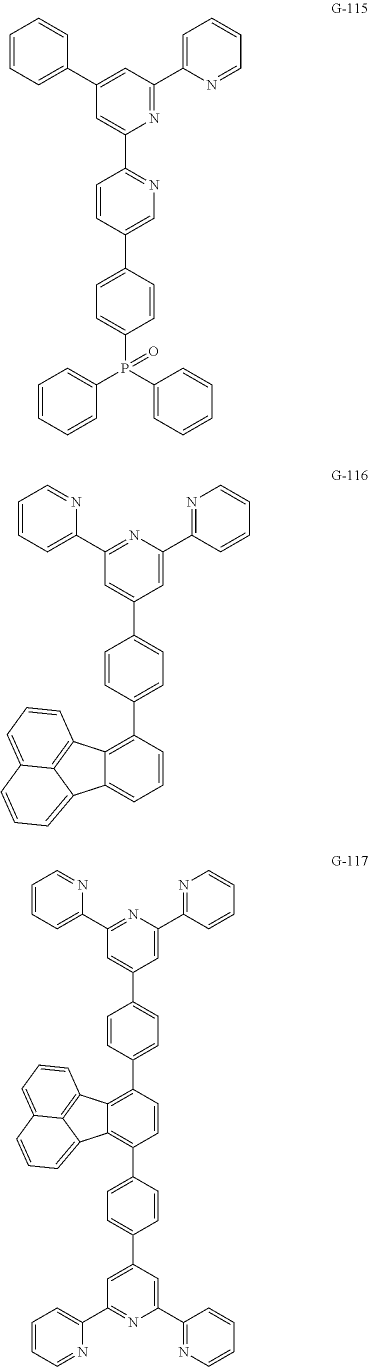 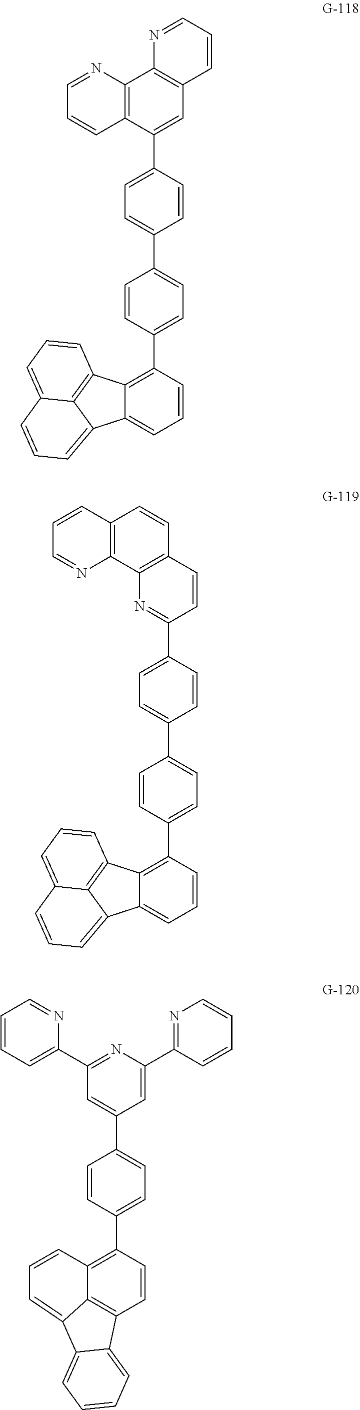 [SMILES: c1cc(-c2ccc(-c3ccc(-c4cc5cccnc5c5ncccc45)cc3)cc2)c2c(c1)-c1cccc3cccc-2c13 ; c1cc(-c2ccc(-c3ccc(-c4ccc5ccc6cccnc6c5n4)cc3)cc2)c2c(c1)-c1cccc3cccc-2c13 ; c1ccc(-c2cc(-c3ccc(-c4ccc5c6c(cccc46)-c4ccccc4-5)cc3)cc(-c3ccccn3)n2)nc1] 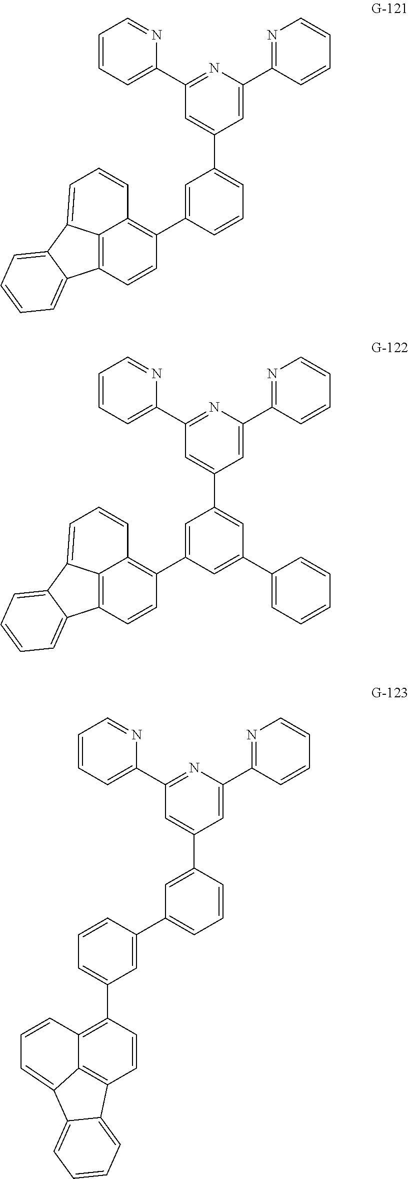 [SMILES: c1ccc(-c2cc(-c3cccc(-c4ccc5c6c(cccc46)-c4ccccc4-5)c3)cc(-c3ccccn3)n2)nc1 ; c1ccc(-c2cc(-c3cc(-c4ccccn4)nc(-c4ccccn4)c3)cc(-c3ccc4c5c(cccc35)-c3ccccc3-4)c2)cc1 ; c1ccc(-c2cc(-c3cccc(-c4cccc(-c5ccc6c7c(cccc57)-c5ccccc5-6)c4)c3)cc(-c3ccccn3)n2)nc1] 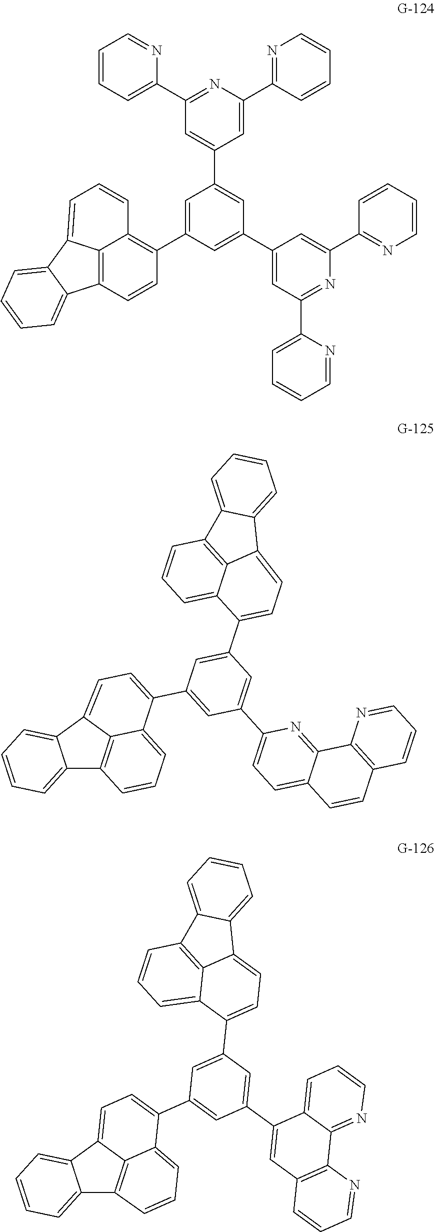 [SMILES: c1ccc(-c2cc(-c3cc(-c4cc(-c5ccccn5)nc(-c5ccccn5)c4)cc(-c4ccc5c6c(cccc46)-c4ccccc4-5)c3)cc(-c3ccccn3)n2)nc1 ; c1ccc2c(c1)-c1cccc3c(-c4cc(-c5ccc6ccc7cccnc7c6n5)cc(-c5ccc6c7c(cccc57)-c5ccccc5-6)c4)ccc-2c13 ; c1ccc2c(c1)-c1cccc3c(-c4cc(-c5ccc6c7c(cccc57)-c5ccccc5-6)cc(-c5cc6cccnc6c6ncccc56)c4)ccc-2c13] 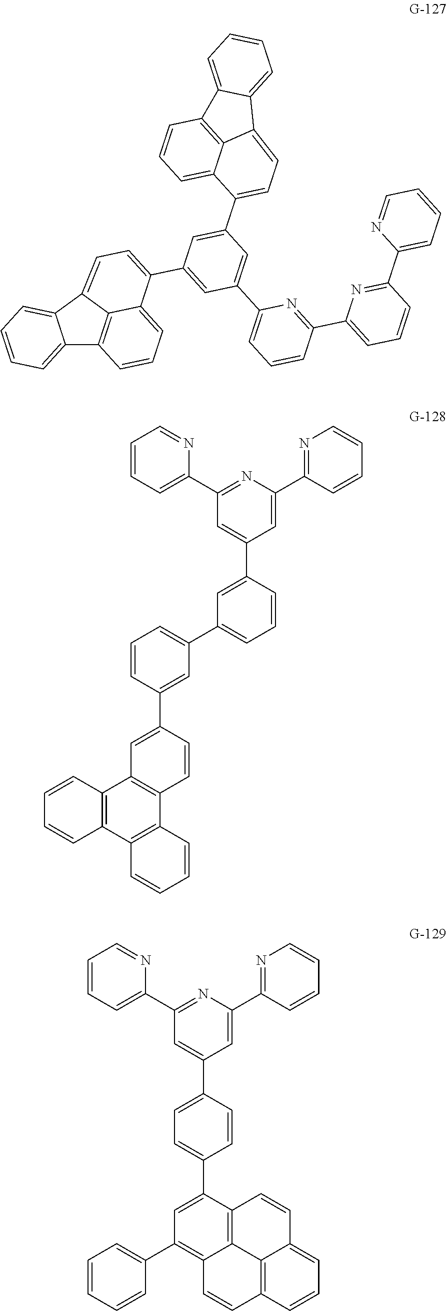 [SMILES: c1ccc(-c2cccc(-c3cccc(-c4cc(-c5ccc6c7c(cccc57)-c5ccccc5-6)cc(-c5ccc6c7c(cccc57)-c5ccccc5-6)c4)n3)n2)nc1 ; c1ccc(-c2cc(-c3cccc(-c4cccc(-c5ccc6c7ccccc7c7ccccc7c6c5)c4)c3)cc(-c3ccccn3)n2)nc1 ; c1ccc(-c2cc(-c3ccc(-c4cc(-c5ccccn5)nc(-c5ccccn5)c4)cc3)c3ccc4cccc5ccc2c3c54)cc1] 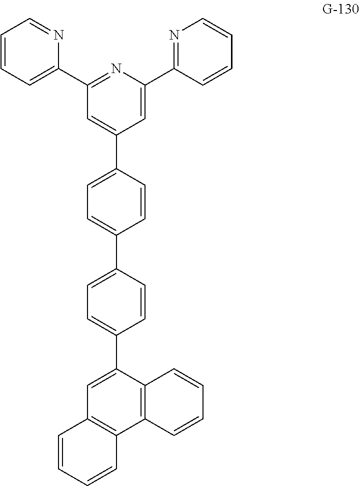 [SMILES: c1ccc(-c2cc(-c3ccc(-c4ccc(-c5cc6ccccc6c6ccccc56)cc4)cc3)cc(-c3ccccn3)n2)nc1] 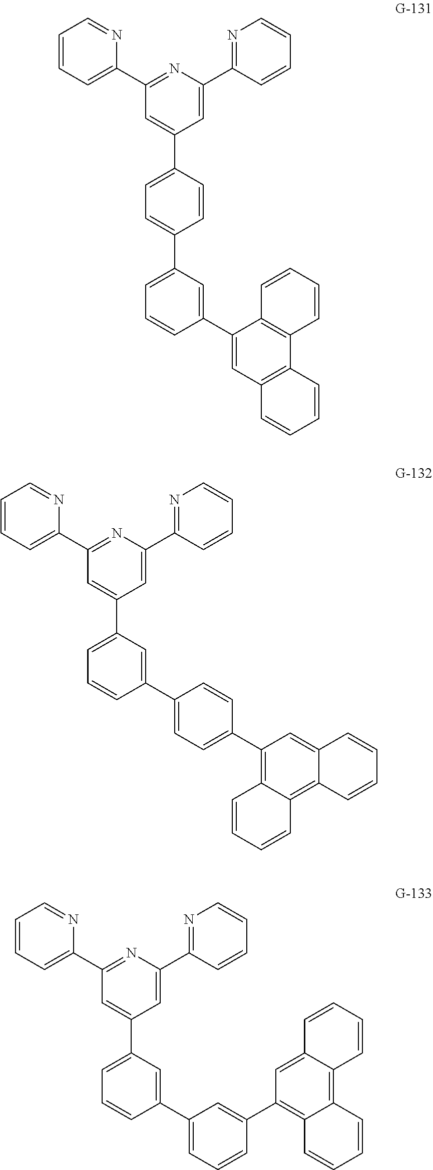 [SMILES: c1ccc(-c2cc(-c3ccc(-c4cccc(-c5cc6ccccc6c6ccccc56)c4)cc3)cc(-c3ccccn3)n2)nc1 ; c1ccc(-c2cc(-c3cccc(-c4ccc(-c5cc6ccccc6c6ccccc56)cc4)c3)cc(-c3ccccn3)n2)nc1 ; c1ccc(-c2cc(-c3cccc(-c4cccc(-c5cc6ccccc6c6ccccc56)c4)c3)cc(-c3ccccn3)n2)nc1] 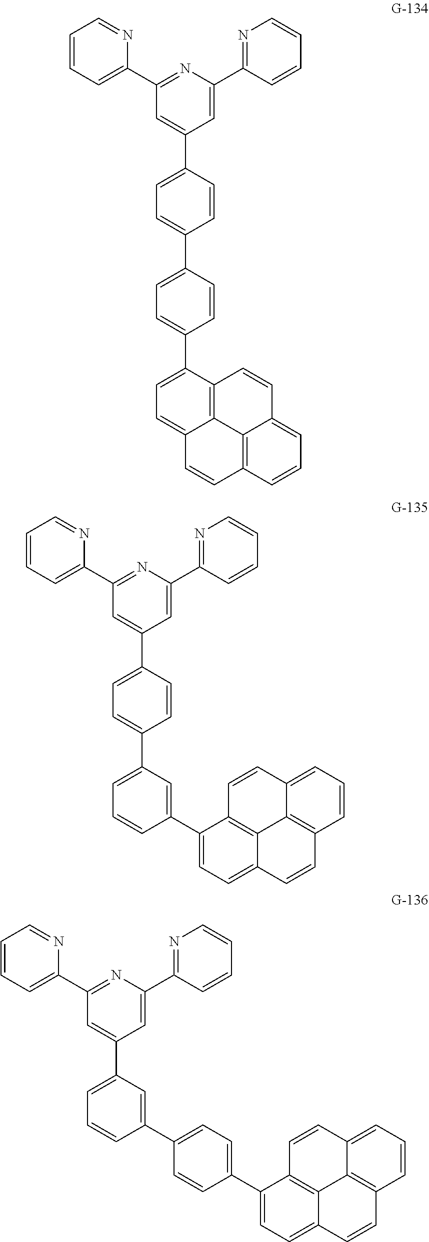 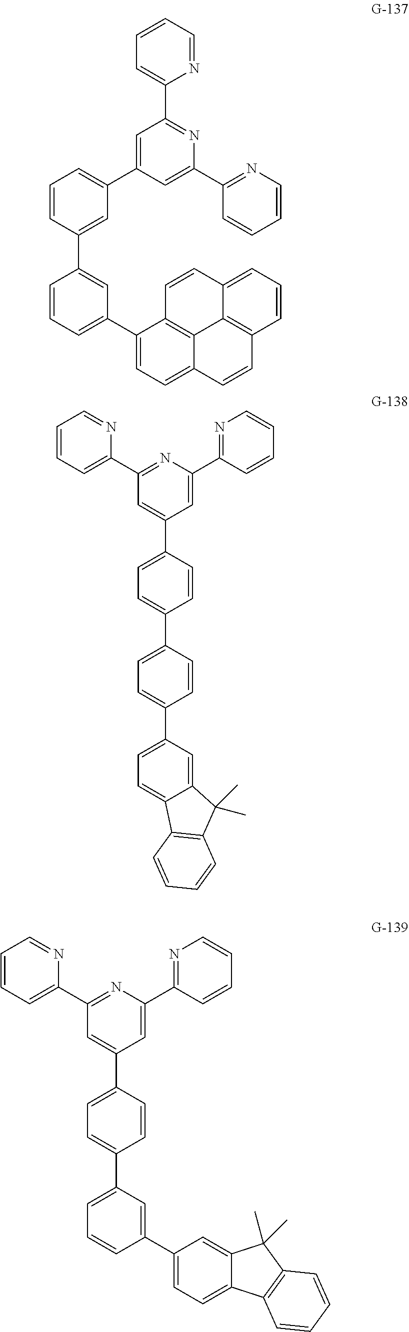 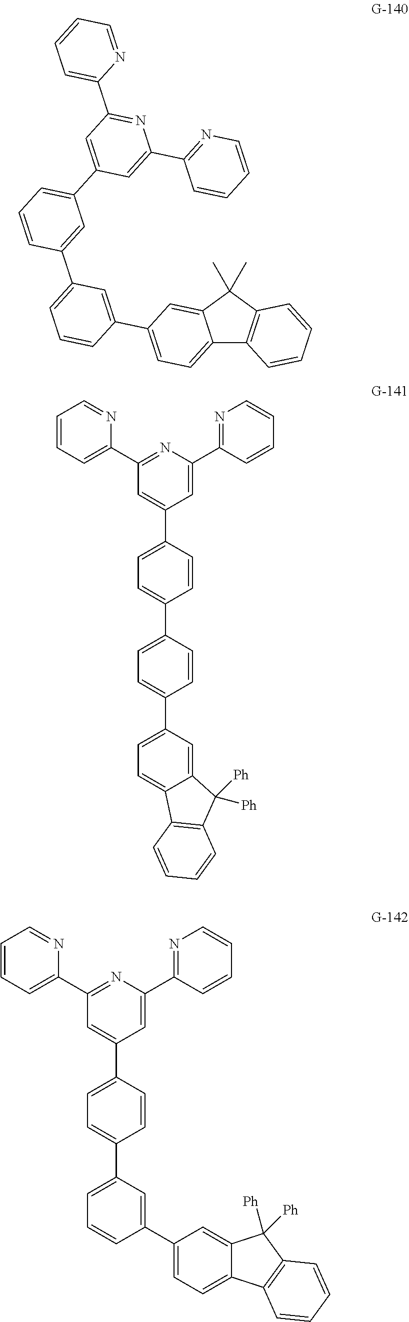 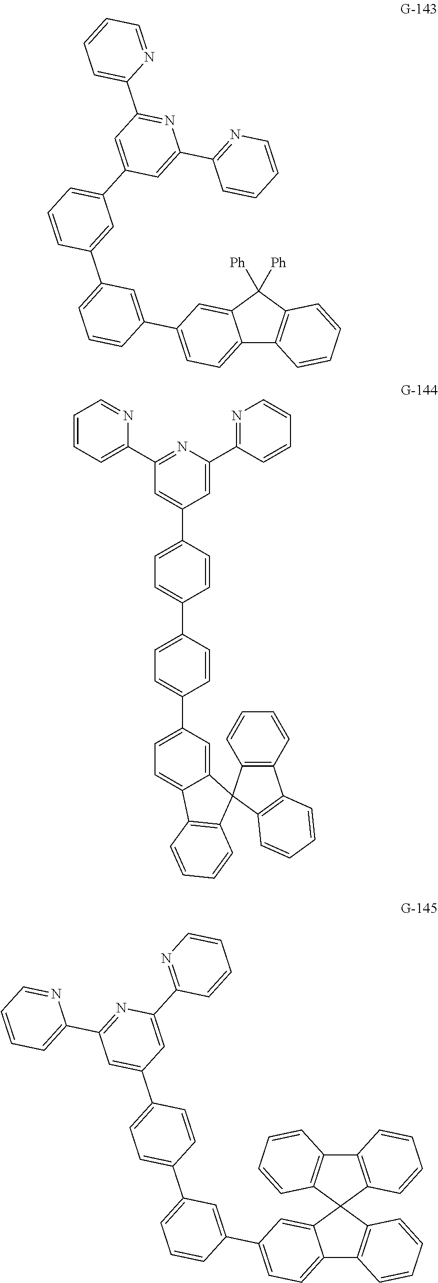 [SMILES: c1ccc(C2(c3ccccc3)c3ccccc3-c3ccc(-c4cccc(-c5cccc(-c6cc(-c7ccccn7)nc(-c7ccccn7)c6)c5)c4)cc32)cc1 ; c1ccc(-c2cc(-c3ccc(-c4ccc(-c5ccc6c(c5)C5(c7ccccc7-c7ccccc75)c5ccccc5-6)cc4)cc3)cc(-c3ccccn3)n2)nc1 ; c1ccc(-c2cc(-c3ccc(-c4cccc(-c5ccc6c(c5)C5(c7ccccc7-c7ccccc75)c5ccccc5-6)c4)cc3)cc(-c3ccccn3)n2)nc1] 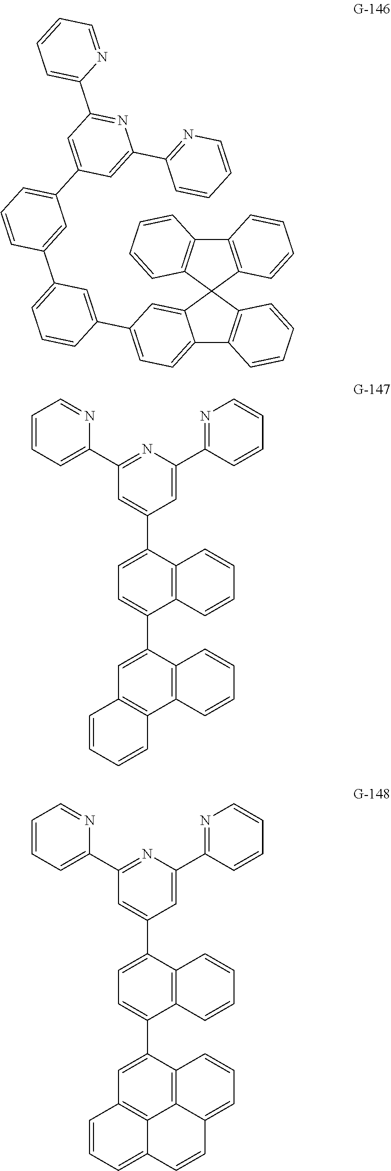 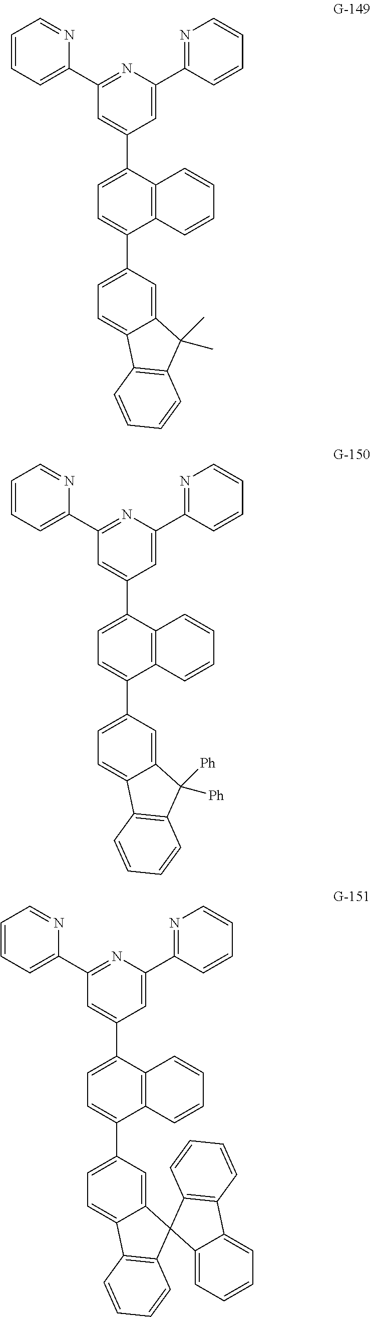 [SMILES: CC1(C)c2ccccc2-c2ccc(-c3ccc(-c4cc(-c5ccccn5)nc(-c5ccccn5)c4)c4ccccc34)cc21 ; c1ccc(C2(c3ccccc3)c3ccccc3-c3ccc(-c4ccc(-c5cc(-c6ccccn6)nc(-c6ccccn6)c5)c5ccccc45)cc32)cc1 ; c1ccc(-c2cc(-c3ccc(-c4ccc5c(c4)C4(c6ccccc6-c6ccccc64)c4ccccc4-5)c4ccccc34)cc(-c3ccccn3)n2)nc1] 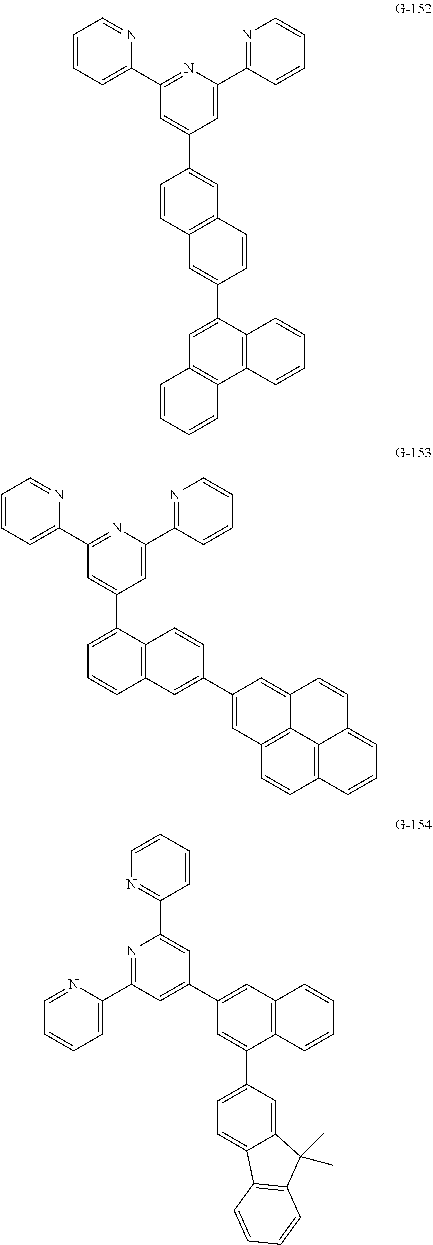 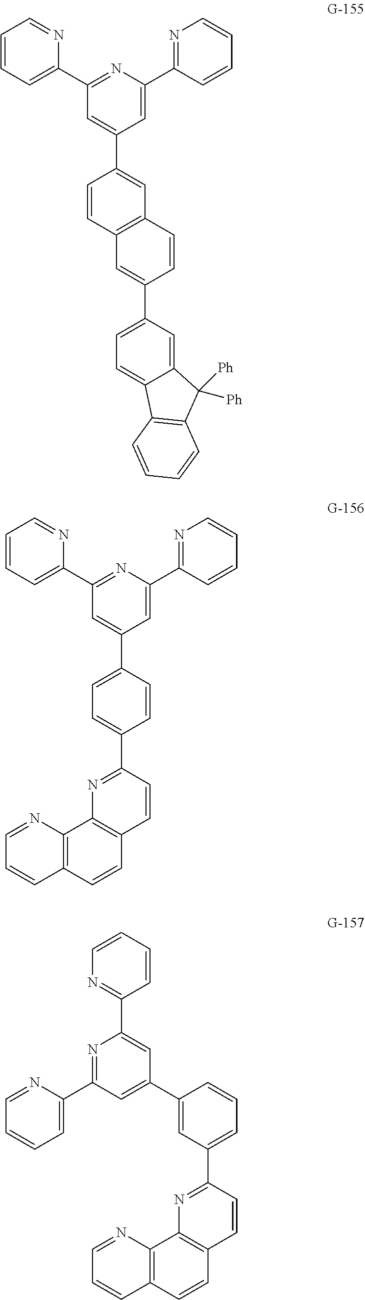 [SMILES: c1ccc(C2(c3ccccc3)c3ccccc3-c3ccc(-c4ccc5cc(-c6cc(-c7ccccn7)nc(-c7ccccn7)c6)ccc5c4)cc32)cc1 ; c1ccc(-c2cc(-c3ccc(-c4ccc5ccc6cccnc6c5n4)cc3)cc(-c3ccccn3)n2)nc1 ; c1ccc(-c2cc(-c3cccc(-c4ccc5ccc6cccnc6c5n4)c3)cc(-c3ccccn3)n2)nc1] 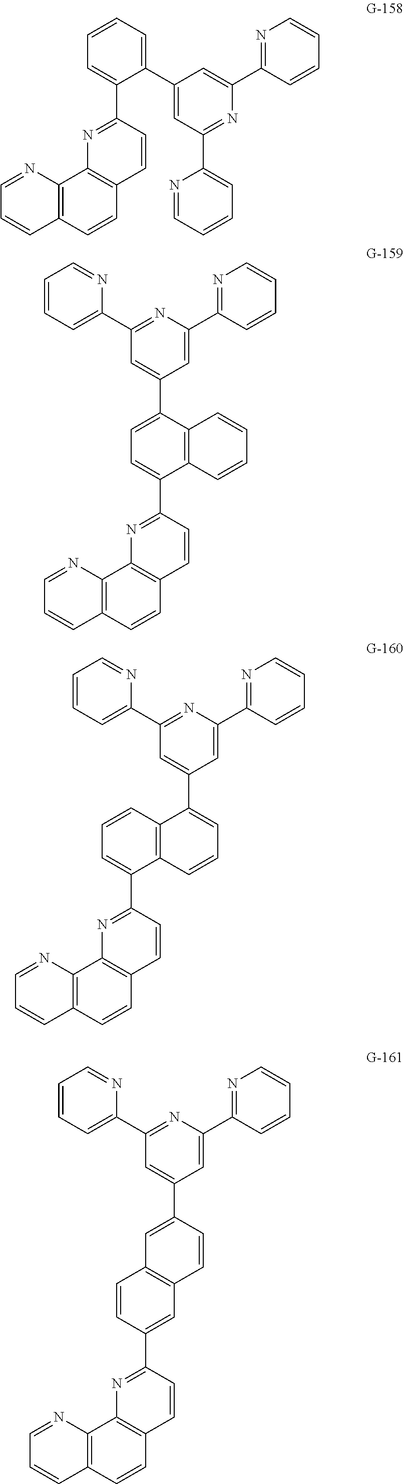 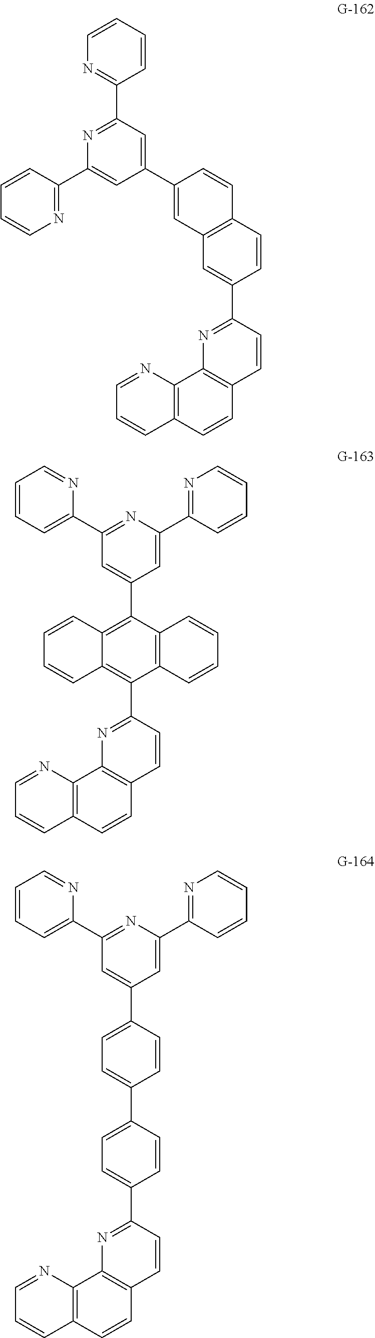 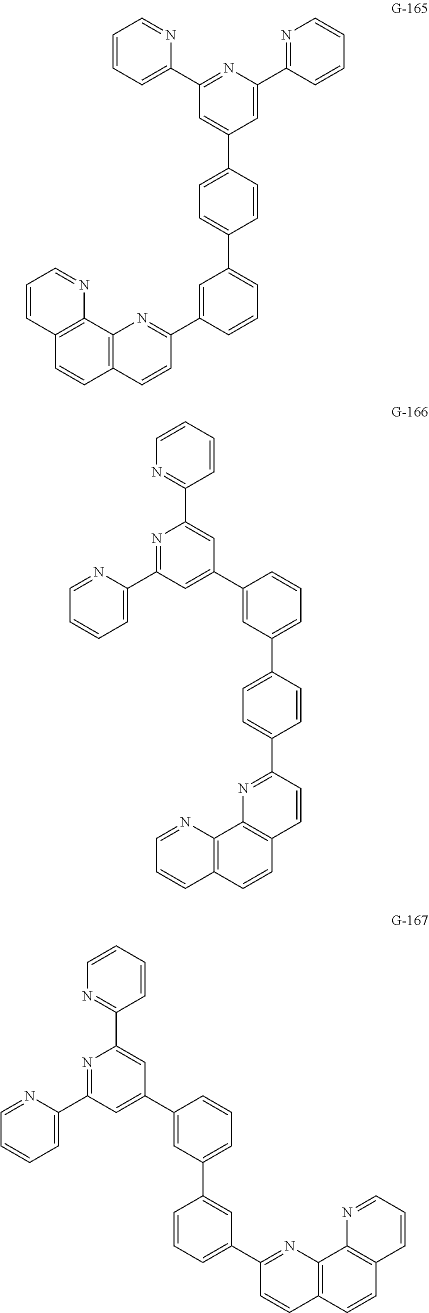 [SMILES: c1ccc(-c2cc(-c3ccc(-c4cccc(-c5ccc6ccc7cccnc7c6n5)c4)cc3)cc(-c3ccccn3)n2)nc1 ; c1ccc(-c2cc(-c3cccc(-c4ccc(-c5ccc6ccc7cccnc7c6n5)cc4)c3)cc(-c3ccccn3)n2)nc1 ; c1ccc(-c2cc(-c3cccc(-c4cccc(-c5ccc6ccc7cccnc7c6n5)c4)c3)cc(-c3ccccn3)n2)nc1] 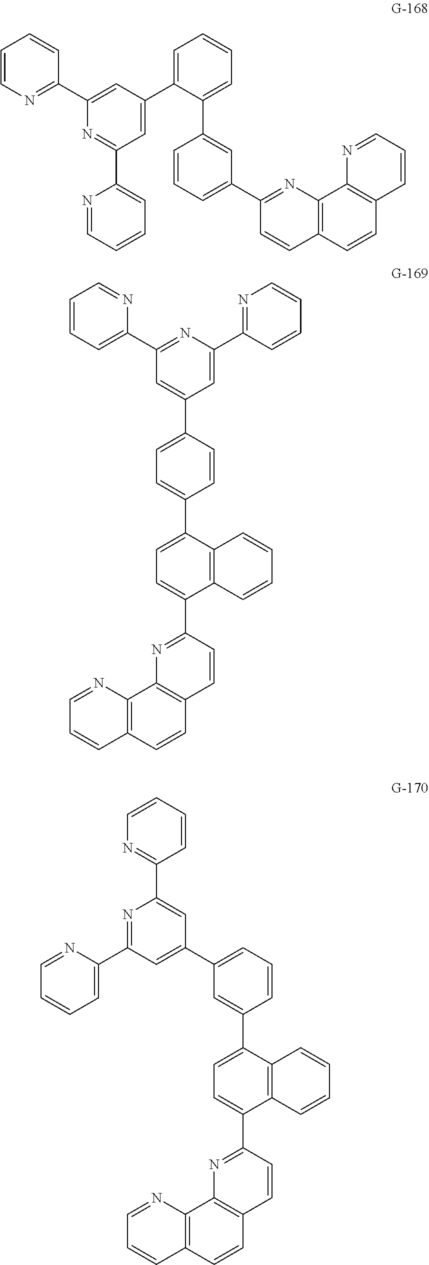 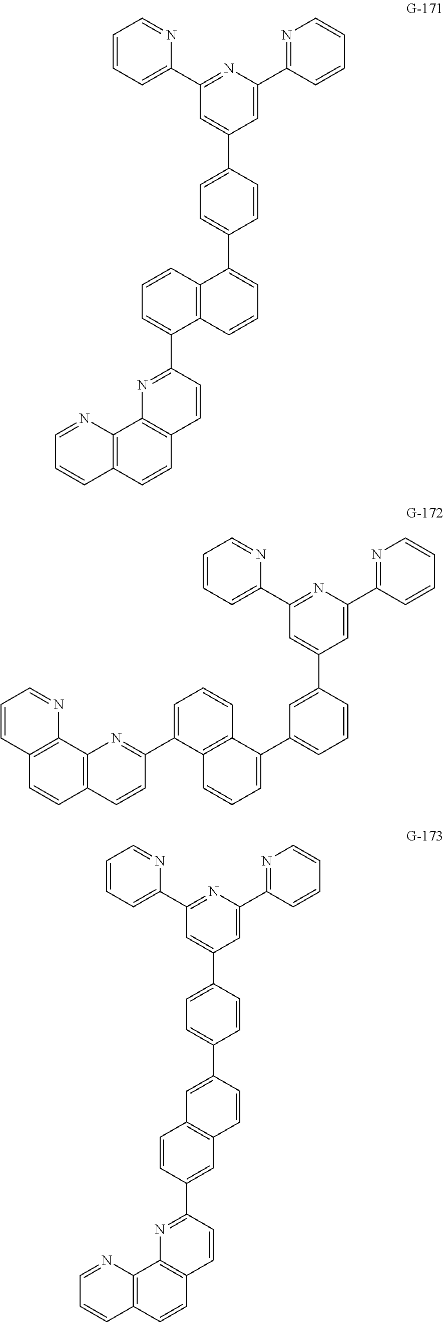 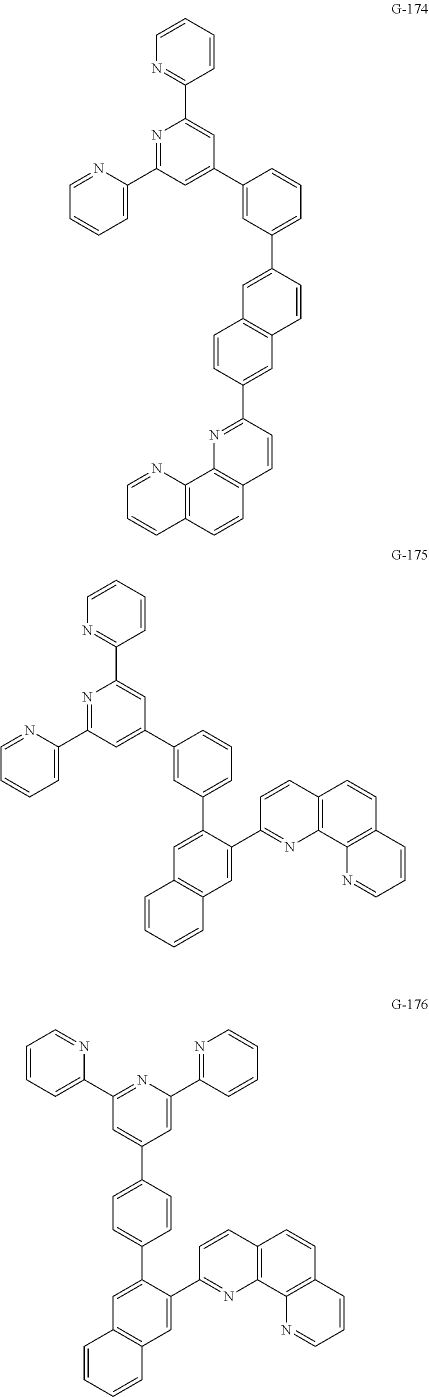 [SMILES: c1ccc(-c2cc(-c3cccc(-c4ccc5cc(-c6ccc7ccc8cccnc8c7n6)ccc5c4)c3)cc(-c3ccccn3)n2)nc1 ; c1ccc(-c2cc(-c3cccc(-c4cc5ccccc5cc4-c4ccc5ccc6cccnc6c5n4)c3)cc(-c3ccccn3)n2)nc1 ; c1ccc(-c2cc(-c3ccc(-c4cc5ccccc5cc4-c4ccc5ccc6cccnc6c5n4)cc3)cc(-c3ccccn3)n2)nc1] 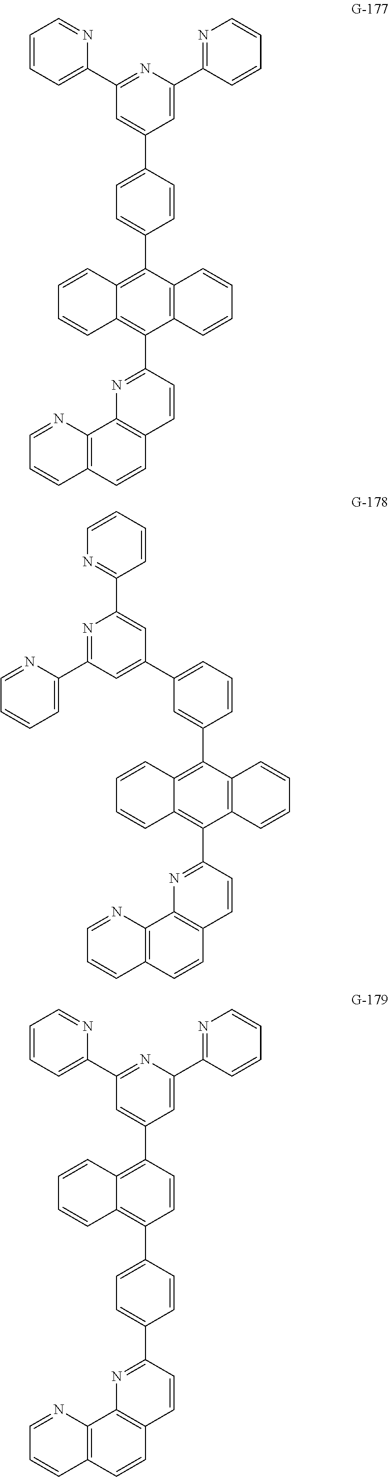 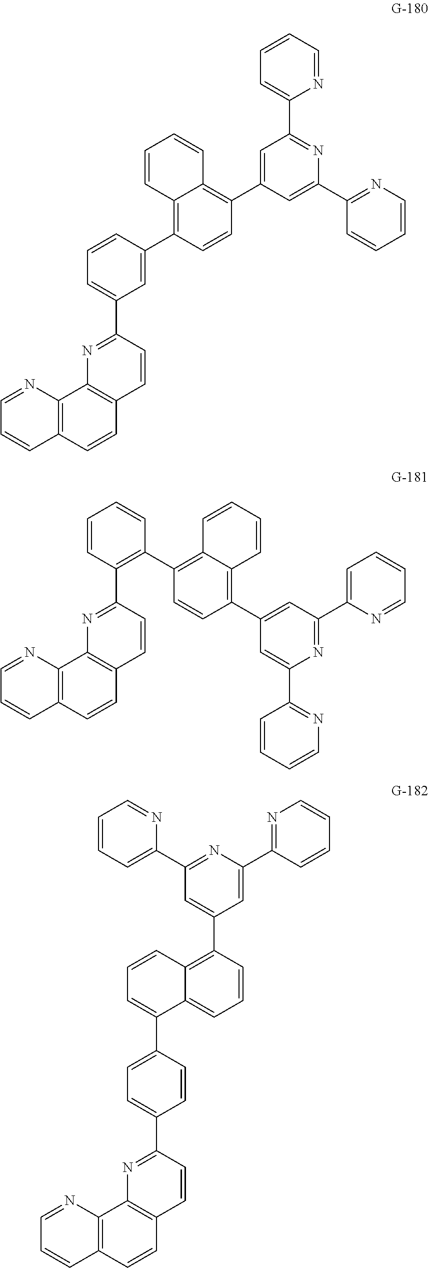 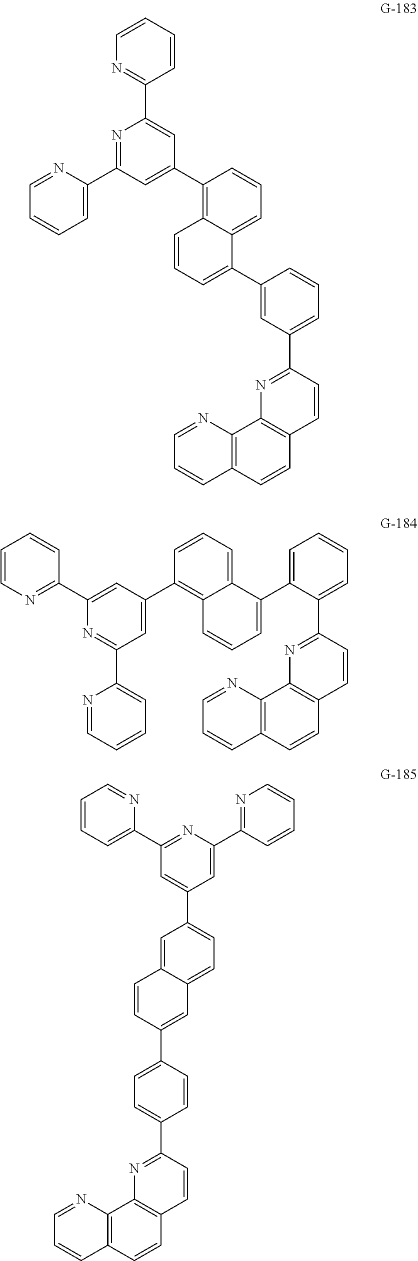 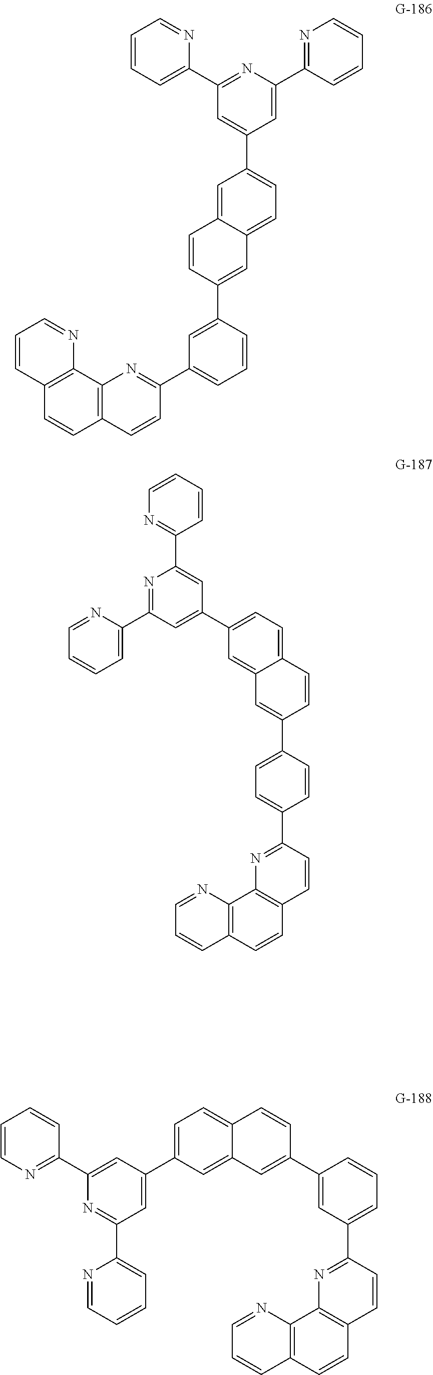 [SMILES: c1ccc(-c2cc(-c3ccc4cc(-c5cccc(-c6ccc7ccc8cccnc8c7n6)c5)ccc4c3)cc(-c3ccccn3)n2)nc1 ; c1ccc(-c2cc(-c3ccc4ccc(-c5ccc(-c6ccc7ccc8cccnc8c7n6)cc5)cc4c3)cc(-c3ccccn3)n2)nc1 ; c1ccc(-c2cc(-c3ccc4ccc(-c5cccc(-c6ccc7ccc8cccnc8c7n6)c5)cc4c3)cc(-c3ccccn3)n2)nc1] 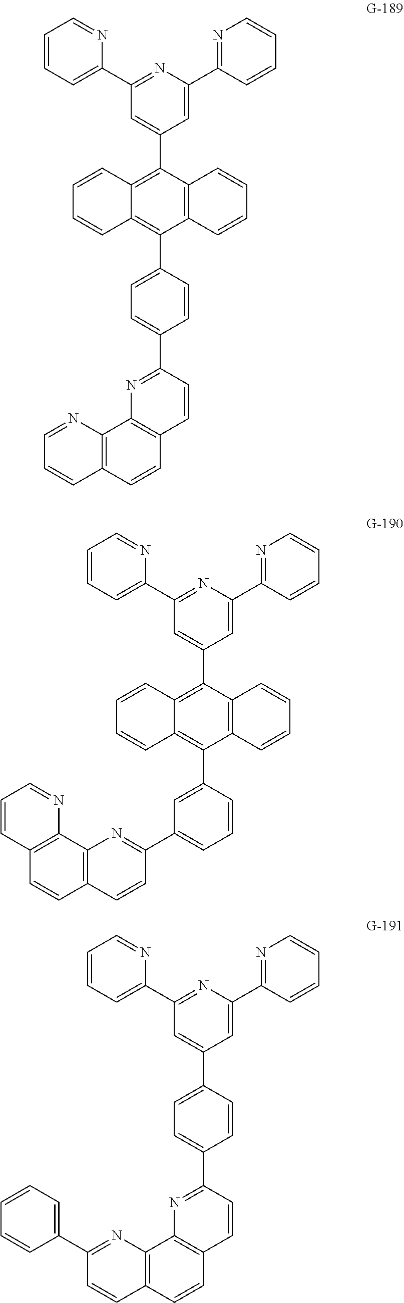 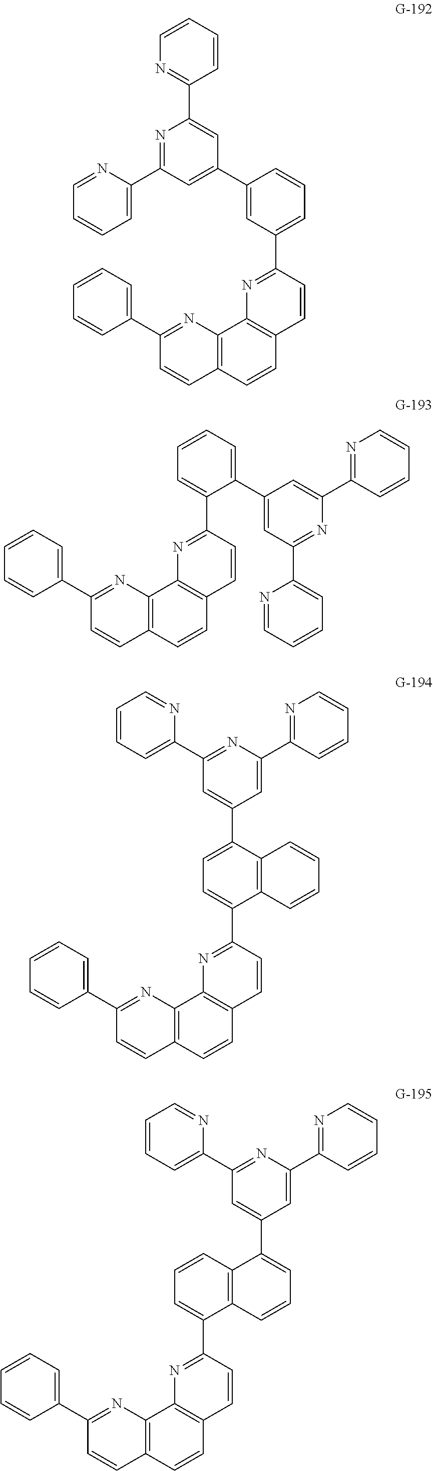 [SMILES: c1ccc(-c2ccc3ccc4ccc(-c5cccc(-c6cc(-c7ccccn7)nc(-c7ccccn7)c6)c5)nc4c3n2)cc1 ; c1ccc(-c2ccc3ccc4ccc(-c5ccccc5-c5cc(-c6ccccn6)nc(-c6ccccn6)c5)nc4c3n2)cc1 ; c1ccc(-c2ccc3ccc4ccc(-c5ccc(-c6cc(-c7ccccn7)nc(-c7ccccn7)c6)c6ccccc56)nc4c3n2)cc1 ; c1ccc(-c2ccc3ccc4ccc(-c5cccc6c(-c7cc(-c8ccccn8)nc(-c8ccccn8)c7)cccc56)nc4c3n2)cc1] 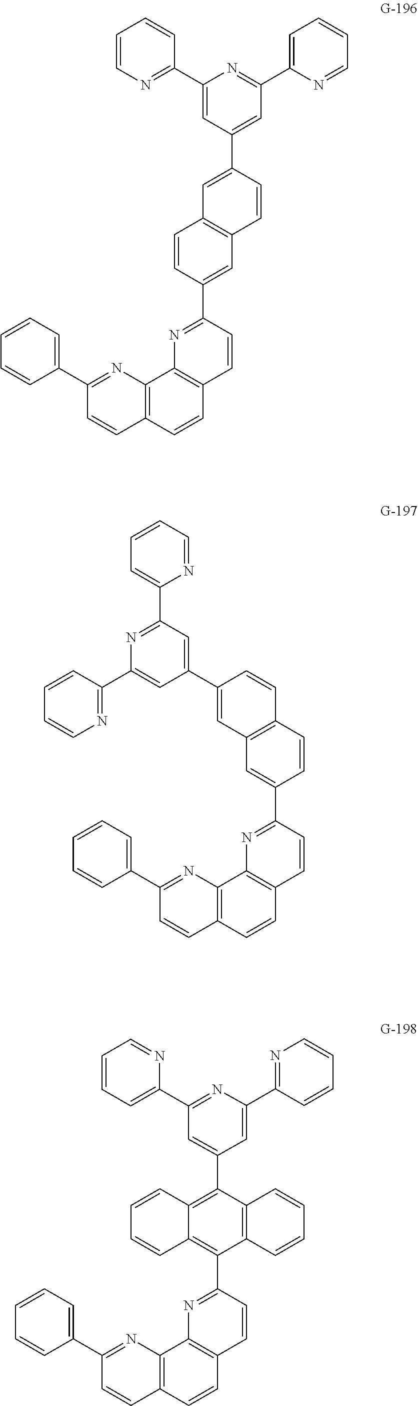 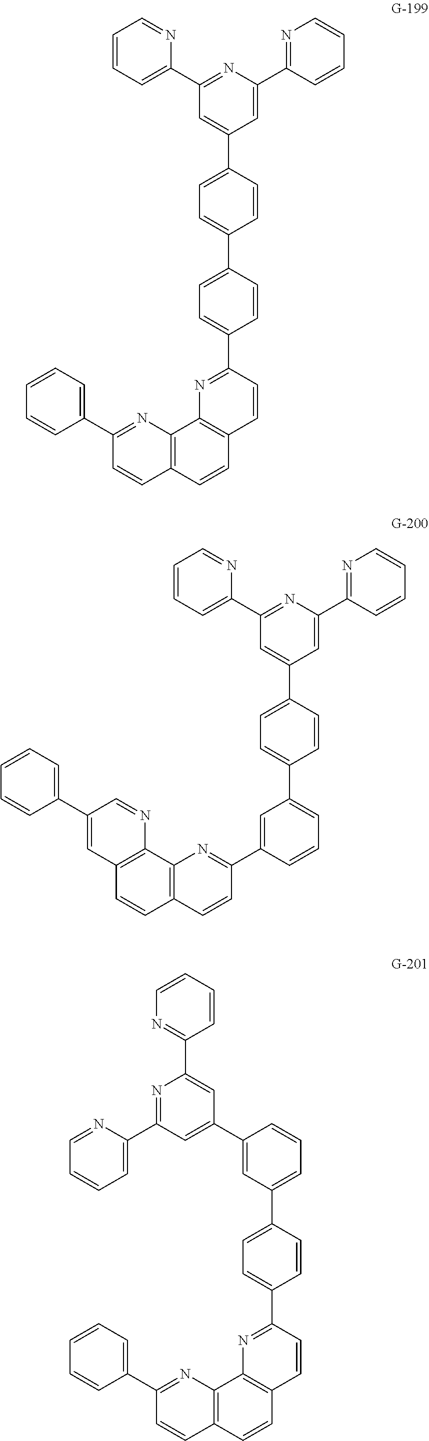 [SMILES: c1ccc(-c2ccc3ccc4ccc(-c5ccc(-c6ccc(-c7cc(-c8ccccn8)nc(-c8ccccn8)c7)cc6)cc5)nc4c3n2)cc1 ; c1ccc(-c2cnc3c(ccc4ccc(-c5cccc(-c6ccc(-c7cc(-c8ccccn8)nc(-c8ccccn8)c7)cc6)c5)nc43)c2)cc1 ; c1ccc(-c2ccc3ccc4ccc(-c5ccc(-c6cccc(-c7cc(-c8ccccn8)nc(-c8ccccn8)c7)c6)cc5)nc4c3n2)cc1] 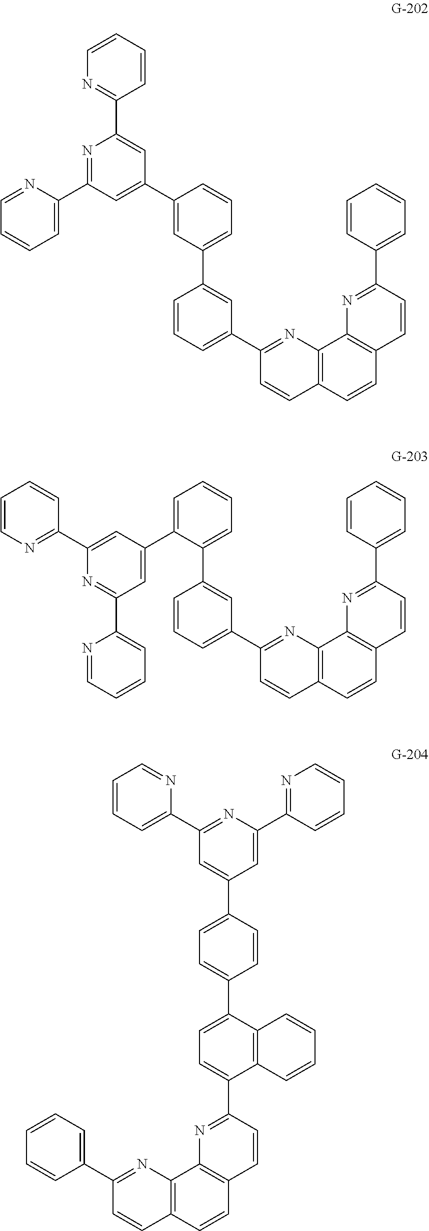 [SMILES: c1ccc(-c2ccc3ccc4ccc(-c5cccc(-c6cccc(-c7cc(-c8ccccn8)nc(-c8ccccn8)c7)c6)c5)nc4c3n2)cc1 ; c1ccc(-c2ccc3ccc4ccc(-c5cccc(-c6ccccc6-c6cc(-c7ccccn7)nc(-c7ccccn7)c6)c5)nc4c3n2)cc1 ; c1ccc(-c2ccc3ccc4ccc(-c5ccc(-c6ccc(-c7cc(-c8ccccn8)nc(-c8ccccn8)c7)cc6)c6ccccc56)nc4c3n2)cc1] 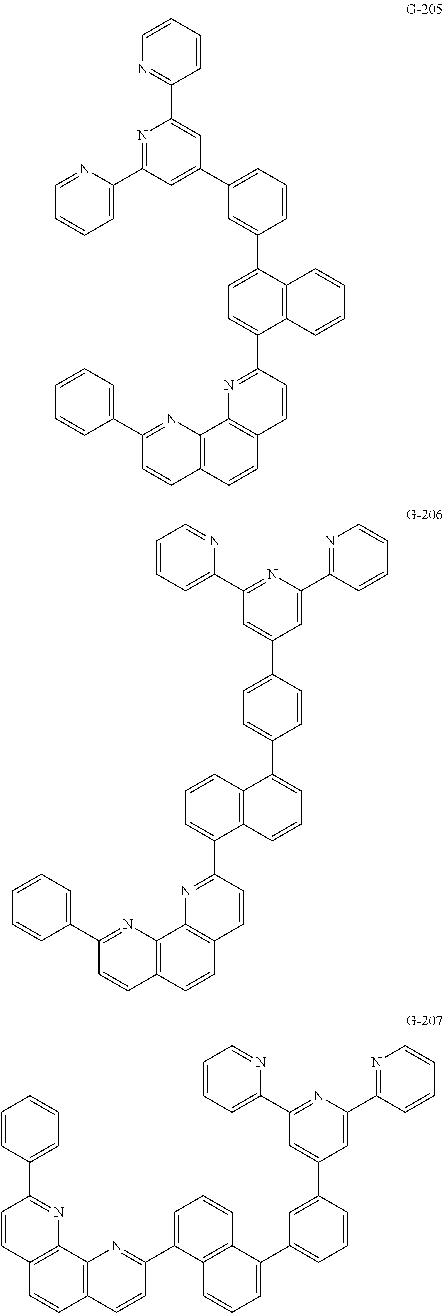 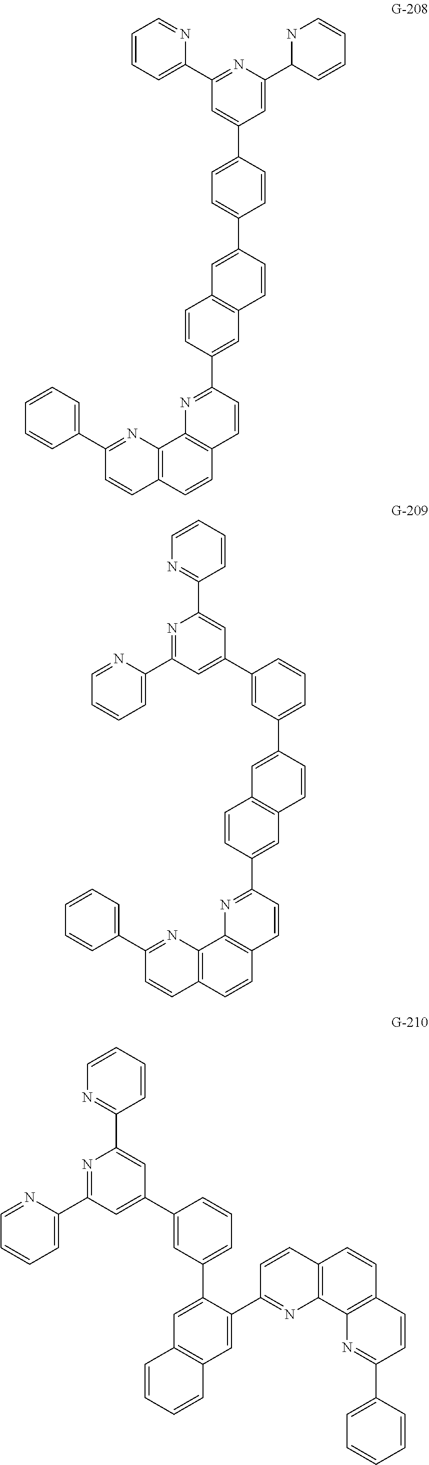 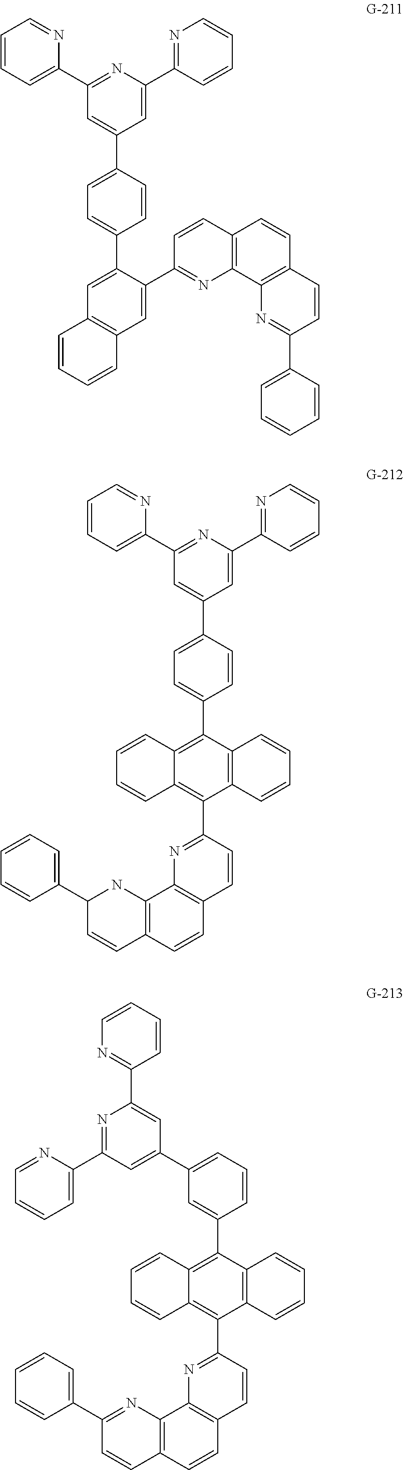 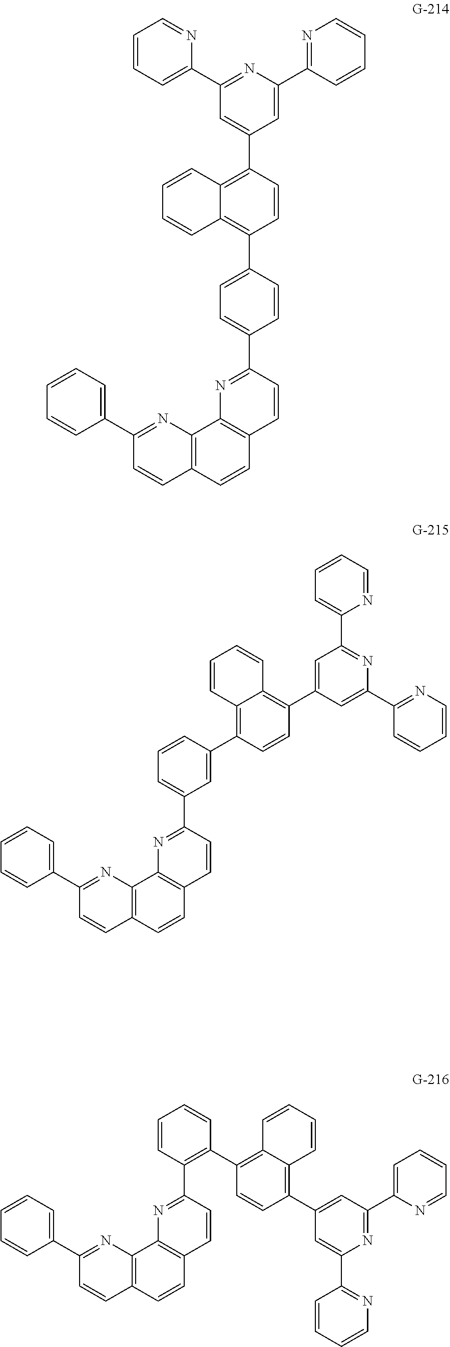 [SMILES: c1ccc(-c2ccc3ccc4ccc(-c5ccc(-c6ccc(-c7cc(-c8ccccn8)nc(-c8ccccn8)c7)c7ccccc67)cc5)nc4c3n2)cc1 ; c1ccc(-c2ccc3ccc4ccc(-c5cccc(-c6ccc(-c7cc(-c8ccccn8)nc(-c8ccccn8)c7)c7ccccc67)c5)nc4c3n2)cc1 ; c1ccc(-c2ccc3ccc4ccc(-c5ccccc5-c5ccc(-c6cc(-c7ccccn7)nc(-c7ccccn7)c6)c6ccccc56)nc4c3n2)cc1] 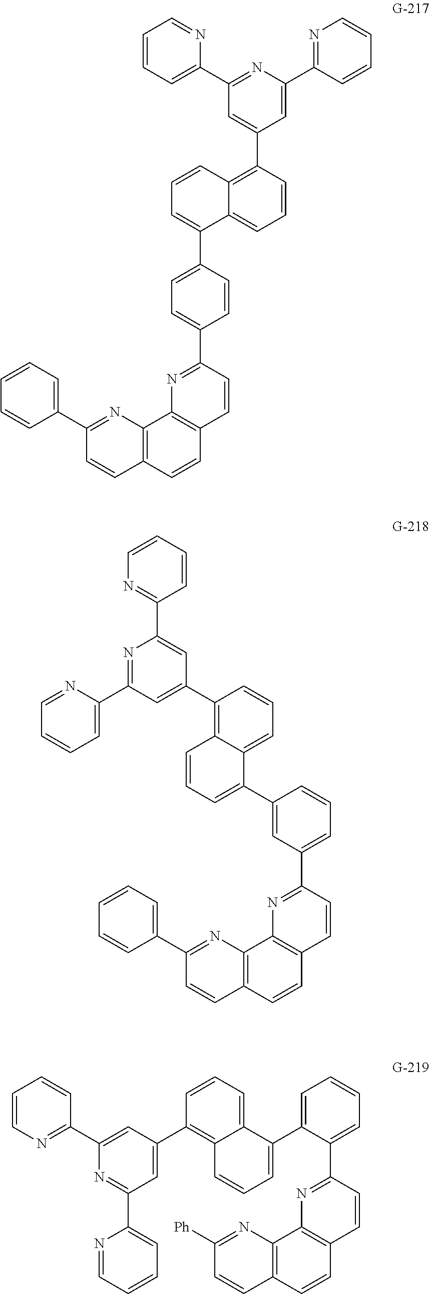 [SMILES: c1ccc(-c2ccc3ccc4ccc(-c5ccc(-c6cccc7c(-c8cc(-c9ccccn9)nc(-c9ccccn9)c8)cccc67)cc5)nc4c3n2)cc1 ; c1ccc(-c2ccc3ccc4ccc(-c5cccc(-c6cccc7c(-c8cc(-c9ccccn9)nc(-c9ccccn9)c8)cccc67)c5)nc4c3n2)cc1 ; c1ccc(-c2ccc3ccc4ccc(-c5ccccc5-c5cccc6c(-c7cc(-c8ccccn8)nc(-c8ccccn8)c7)cccc56)nc4c3n2)cc1] 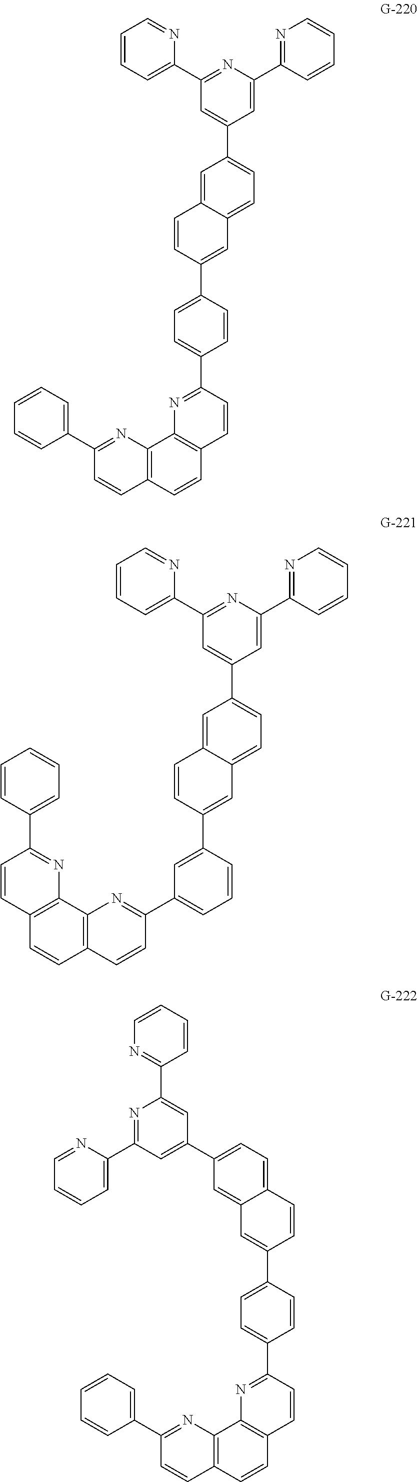 [SMILES: c1ccc(-c2ccc3ccc4ccc(-c5ccc(-c6ccc7cc(-c8cc(-c9ccccn9)nc(-c9ccccn9)c8)ccc7c6)cc5)nc4c3n2)cc1 ; c1ccc(-c2ccc3ccc4ccc(-c5cccc(-c6ccc7cc(-c8cc(-c9ccccn9)nc(-c9ccccn9)c8)ccc7c6)c5)nc4c3n2)cc1 ; c1ccc(-c2ccc3ccc4ccc(-c5ccc(-c6ccc7ccc(-c8cc(-c9ccccn9)nc(-c9ccccn9)c8)cc7c6)cc5)nc4c3n2)cc1] 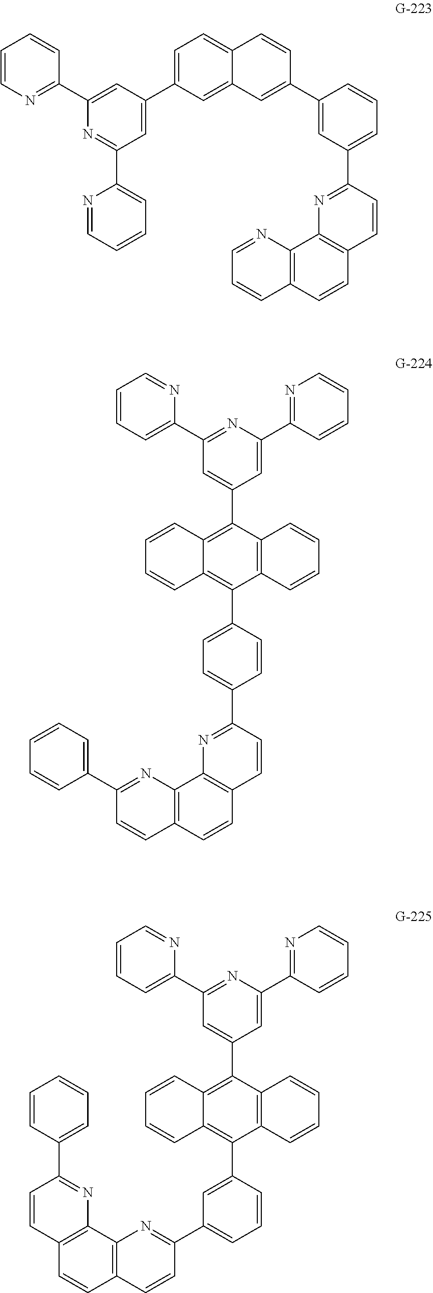 [SMILES: c1ccc(-c2cc(-c3ccc4ccc(-c5cccc(-c6ccc7ccc8cccnc8c7n6)c5)cc4c3)cc(-c3ccccn3)n2)nc1 ; c1ccc(-c2ccc3ccc4ccc(-c5ccc(-c6c7ccccc7c(-c7cc(-c8ccccn8)nc(-c8ccccn8)c7)c7ccccc67)cc5)nc4c3n2)cc1 ; c1ccc(-c2ccc3ccc4ccc(-c5cccc(-c6c7ccccc7c(-c7cc(-c8ccccn8)nc(-c8ccccn8)c7)c7ccccc67)c5)nc4c3n2)cc1] 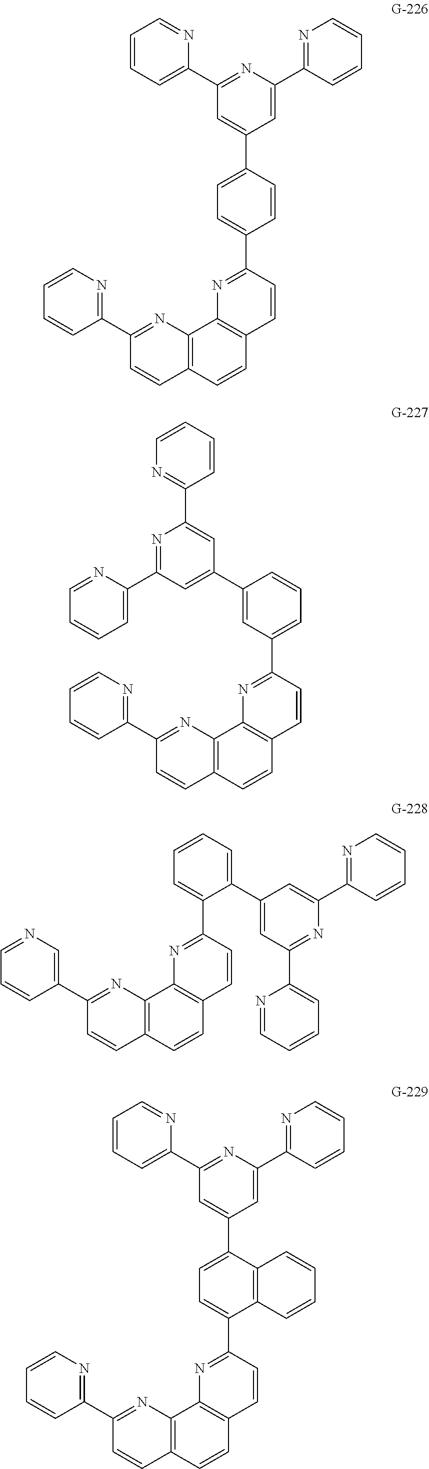 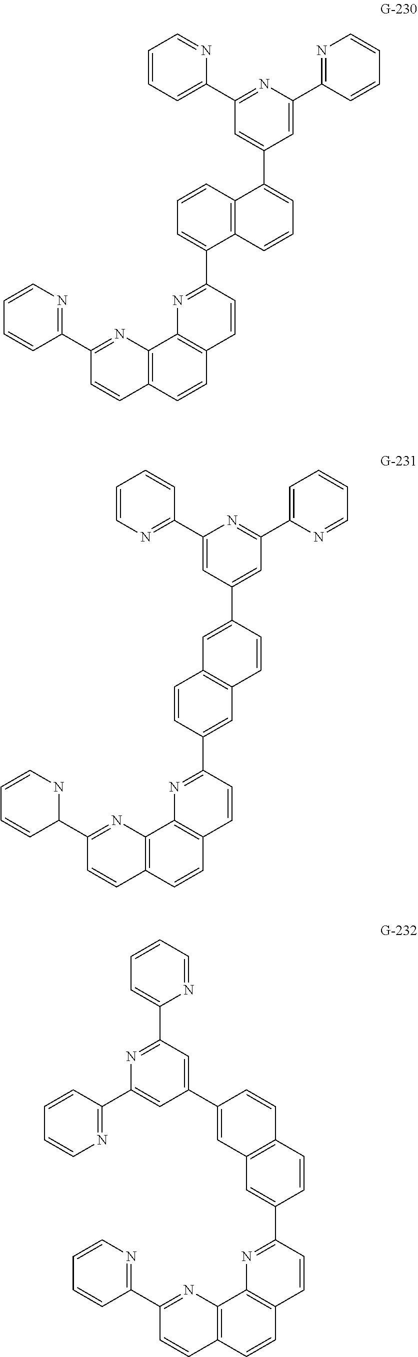 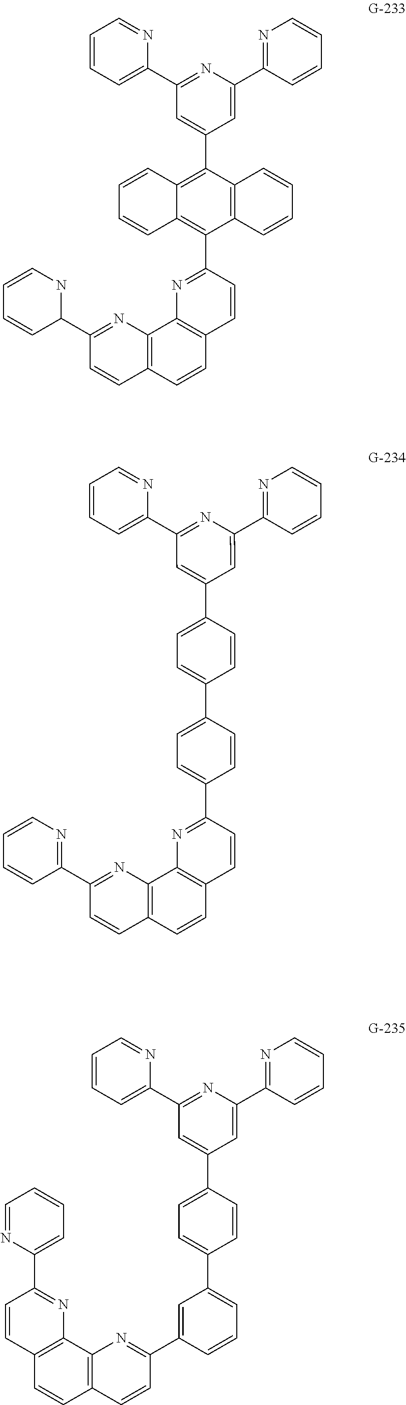 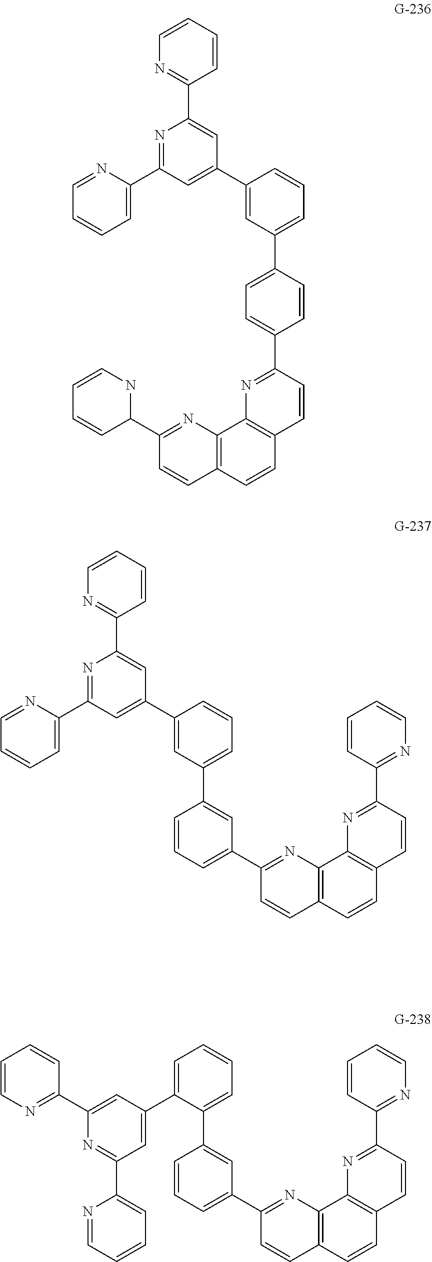 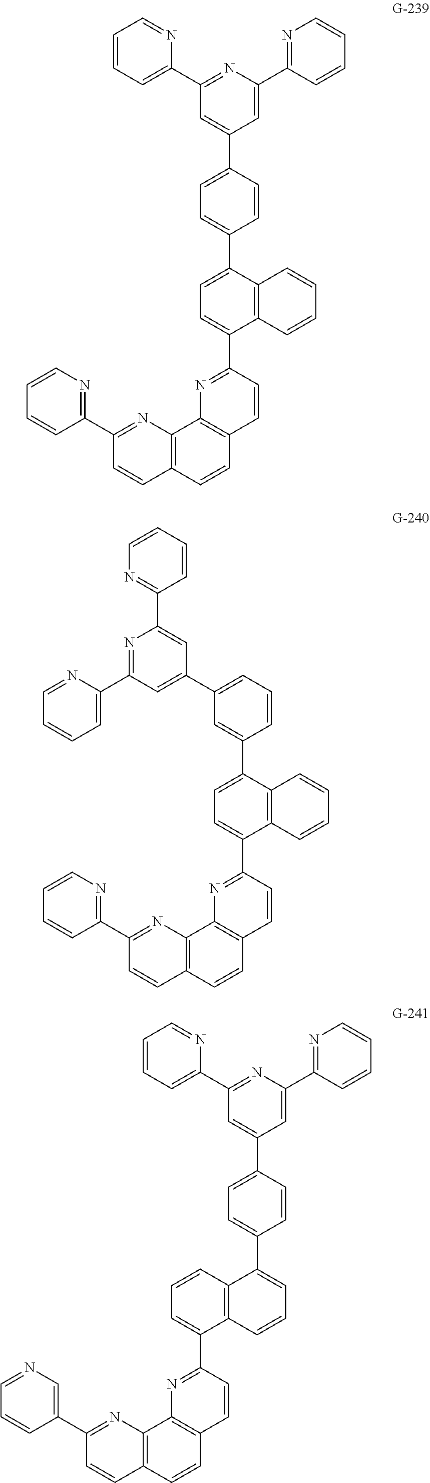 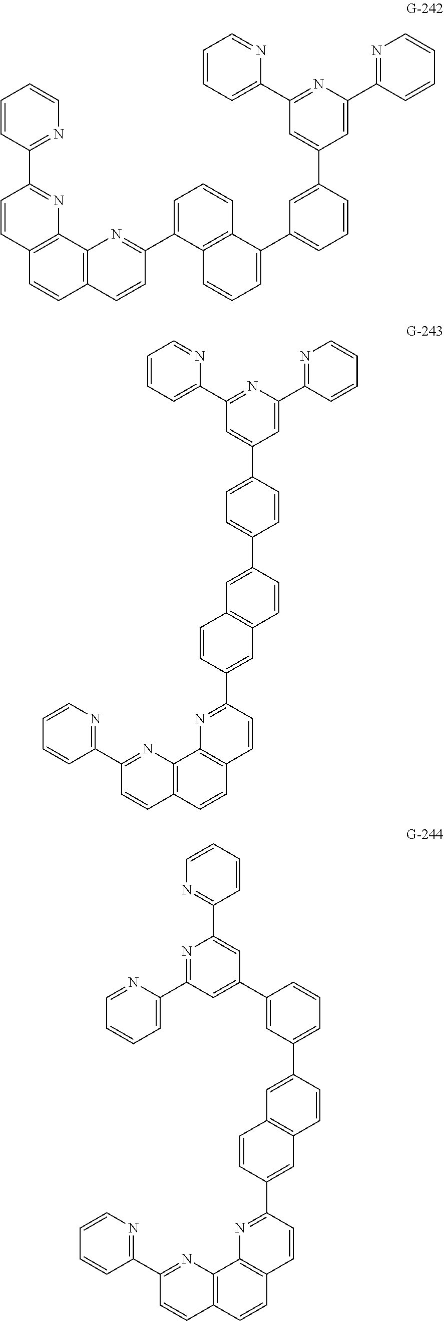 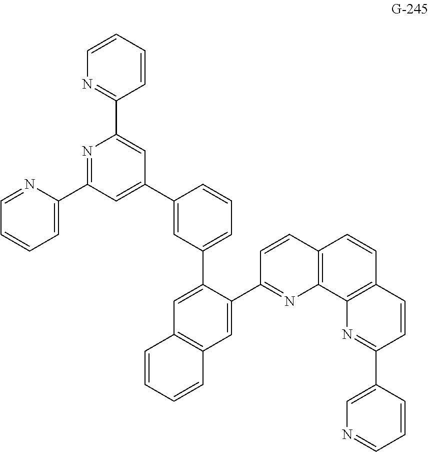 [SMILES: c1ccc(-c2cc(-c3cccc(-c4cc5ccccc5cc4-c4ccc5ccc6ccc(-c7cccnc7)nc6c5n4)c3)cc(-c3ccccn3)n2)nc1] 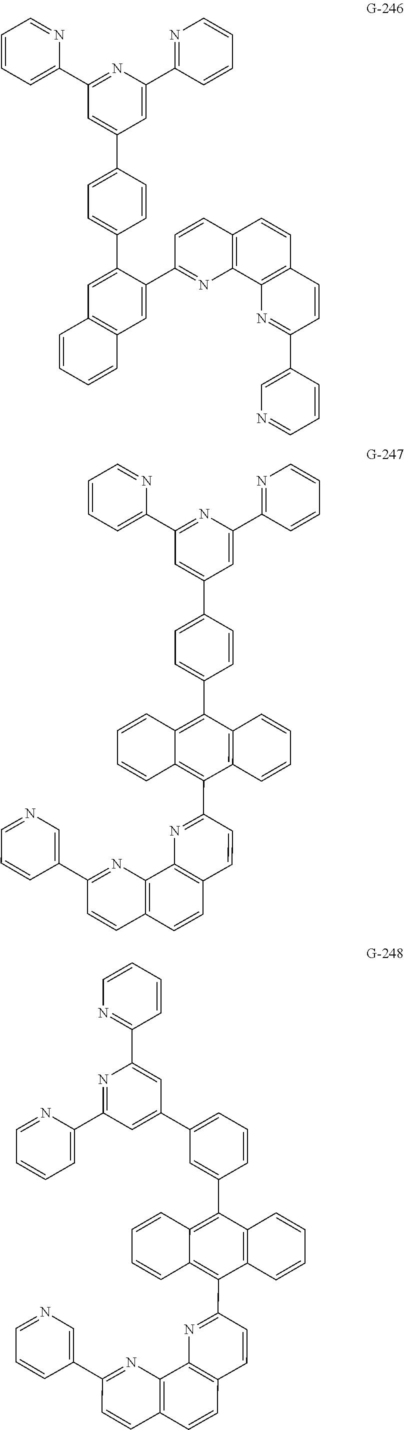 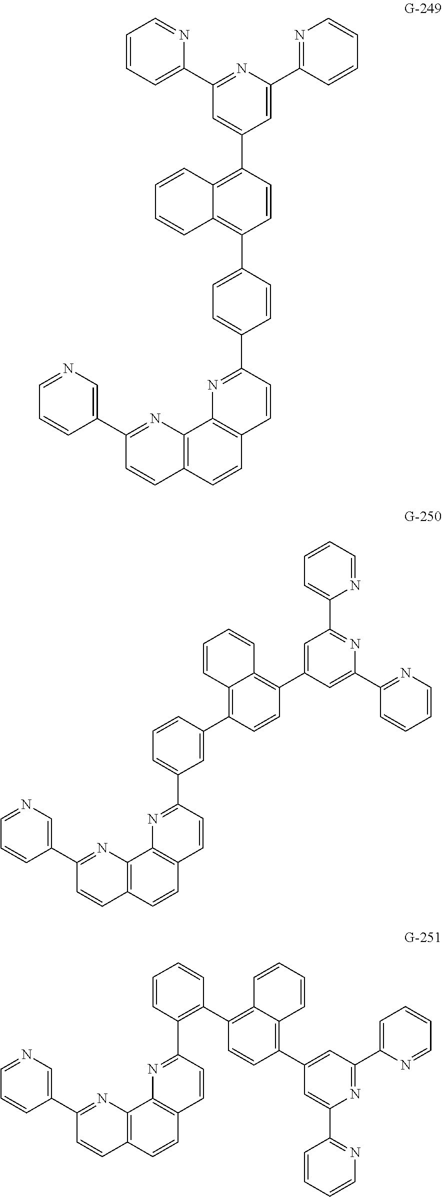 [SMILES: c1ccc(-c2cc(-c3ccc(-c4ccc(-c5ccc6ccc7ccc(-c8cccnc8)nc7c6n5)cc4)c4ccccc34)cc(-c3ccccn3)n2)nc1 ; c1ccc(-c2cc(-c3ccc(-c4cccc(-c5ccc6ccc7ccc(-c8cccnc8)nc7c6n5)c4)c4ccccc34)cc(-c3ccccn3)n2)nc1 ; c1ccc(-c2cc(-c3ccc(-c4ccccc4-c4ccc5ccc6ccc(-c7cccnc7)nc6c5n4)c4ccccc34)cc(-c3ccccn3)n2)nc1] 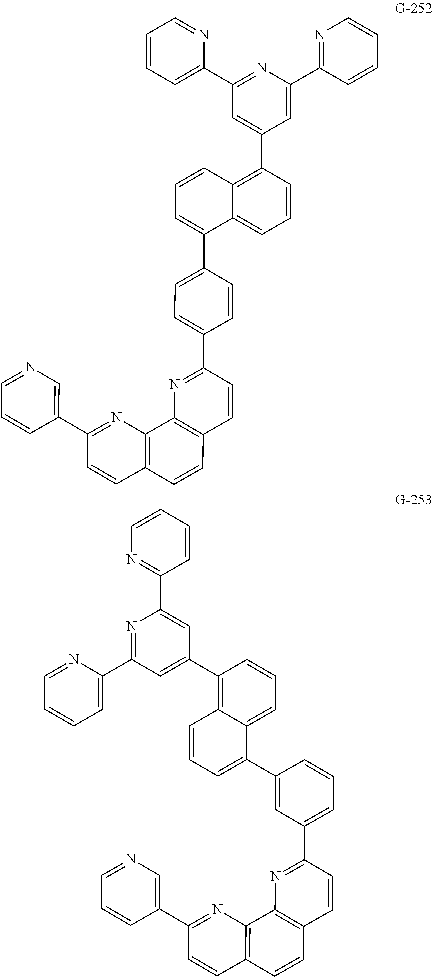 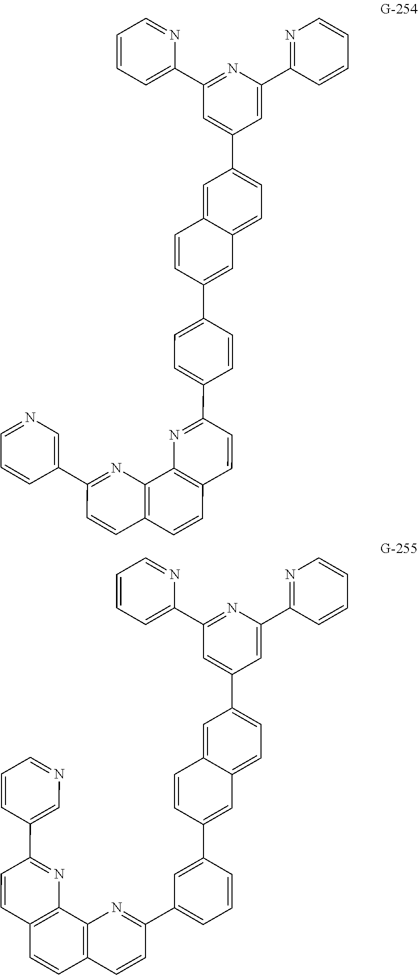 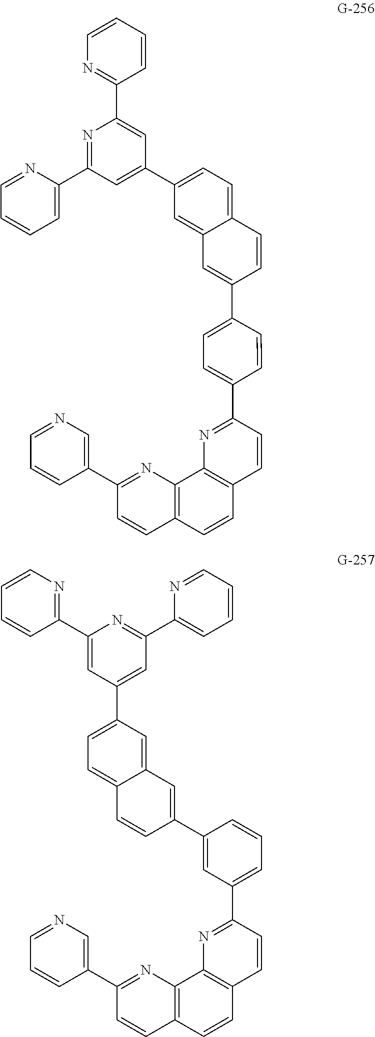 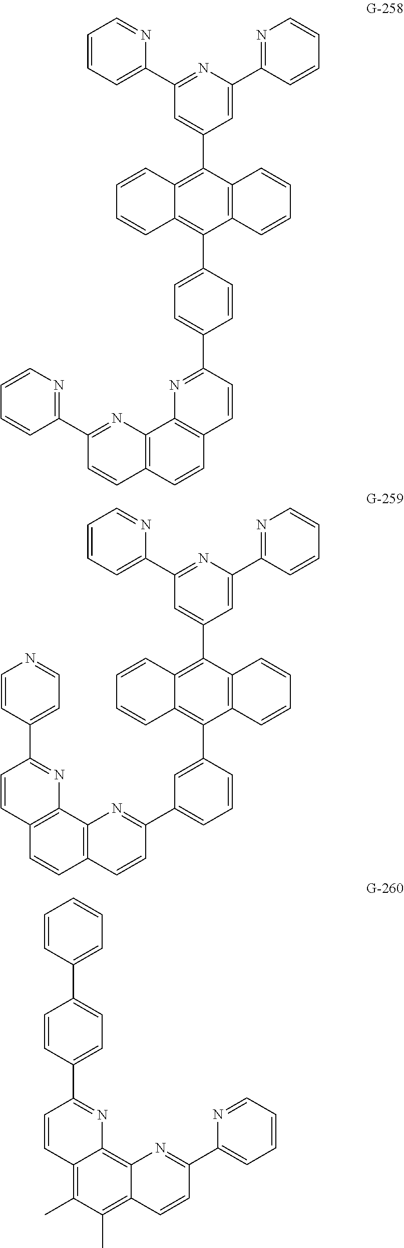 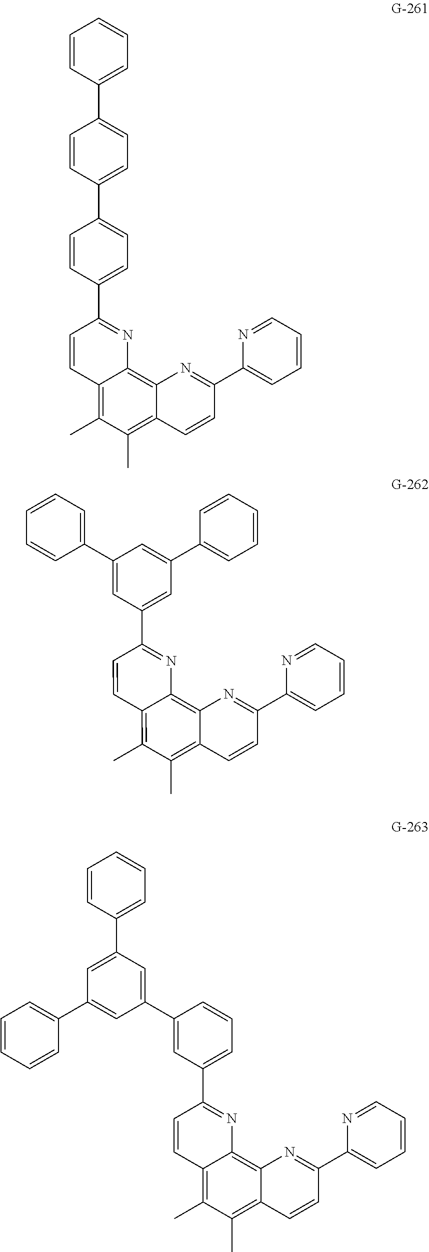 [SMILES: Cc1c(C)c2ccc(-c3ccccn3)nc2c2nc(-c3ccc(-c4ccc(-c5ccccc5)cc4)cc3)ccc12 ; Cc1c(C)c2ccc(-c3ccccn3)nc2c2nc(-c3cc(-c4ccccc4)cc(-c4ccccc4)c3)ccc12 ; Cc1c(C)c2ccc(-c3ccccn3)nc2c2nc(-c3cccc(-c4cc(-c5ccccc5)cc(-c5ccccc5)c4)c3)ccc12] 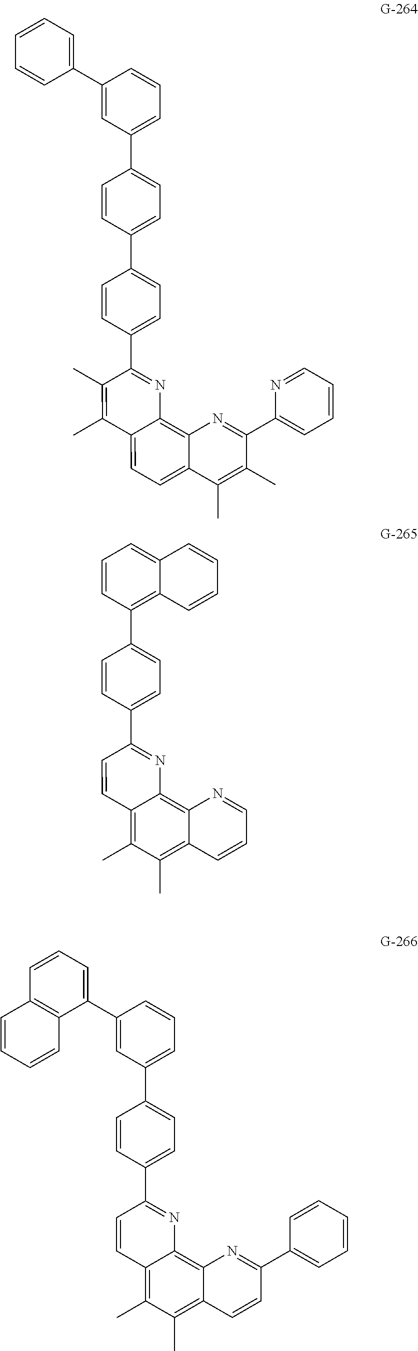 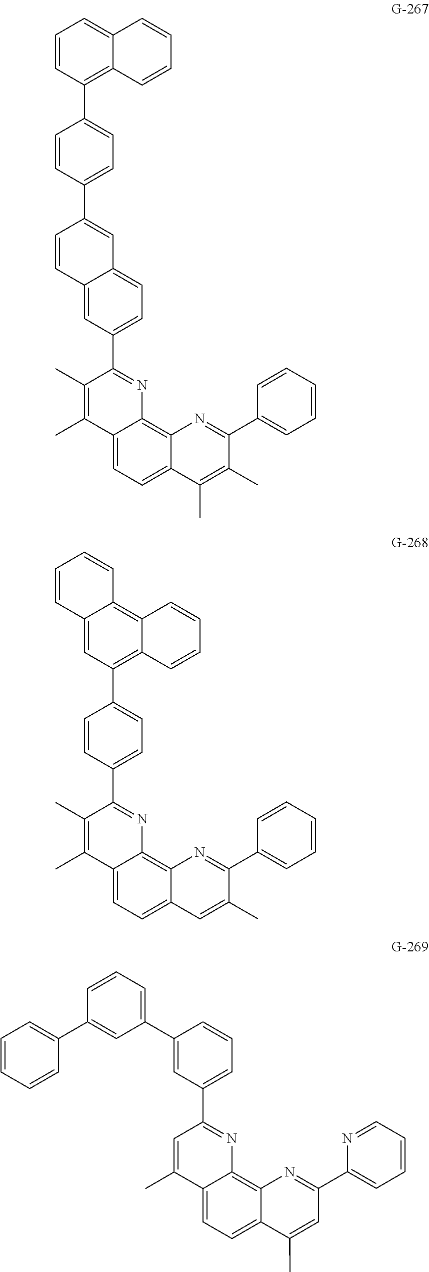 [SMILES: Cc1c(-c2ccccc2)nc2c(ccc3c(C)c(C)c(-c4ccc5cc(-c6ccc(-c7cccc8ccccc78)cc6)ccc5c4)nc32)c1C ; Cc1cc2ccc3c(C)c(C)c(-c4ccc(-c5cc6ccccc6c6ccccc56)cc4)nc3c2nc1-c1ccccc1 ; Cc1cc(-c2cccc(-c3cccc(-c4ccccc4)c3)c2)nc2c1ccc1c(C)cc(-c3ccccn3)nc12] 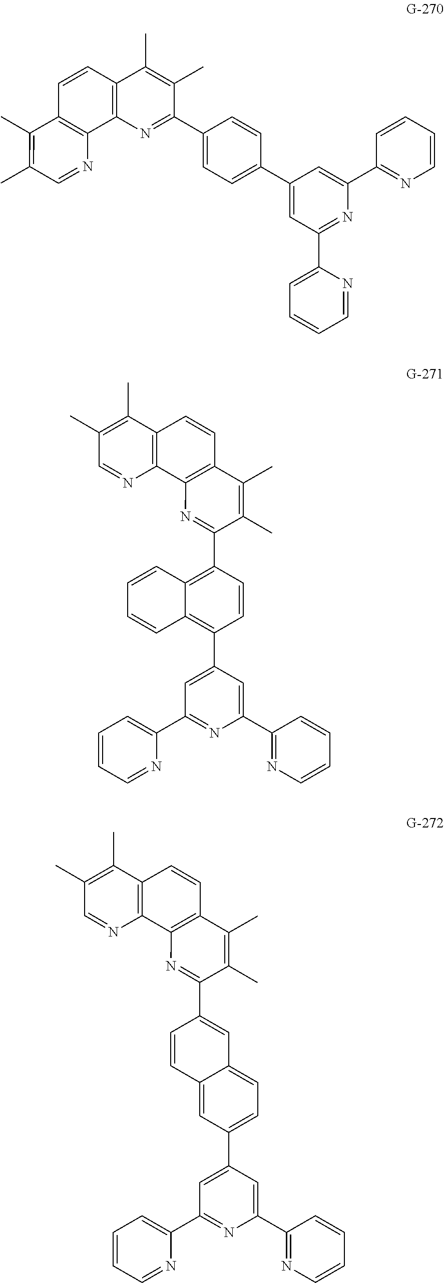 [SMILES: Cc1cnc2c(ccc3c(C)c(C)c(-c4ccc(-c5cc(-c6ccccn6)nc(-c6ccccn6)c5)cc4)nc32)c1C ; Cc1cnc2c(ccc3c(C)c(C)c(-c4ccc(-c5cc(-c6ccccn6)nc(-c6ccccn6)c5)c5ccccc45)nc32)c1C ; Cc1cnc2c(ccc3c(C)c(C)c(-c4ccc5cc(-c6cc(-c7ccccn7)nc(-c7ccccn7)c6)ccc5c4)nc32)c1C] 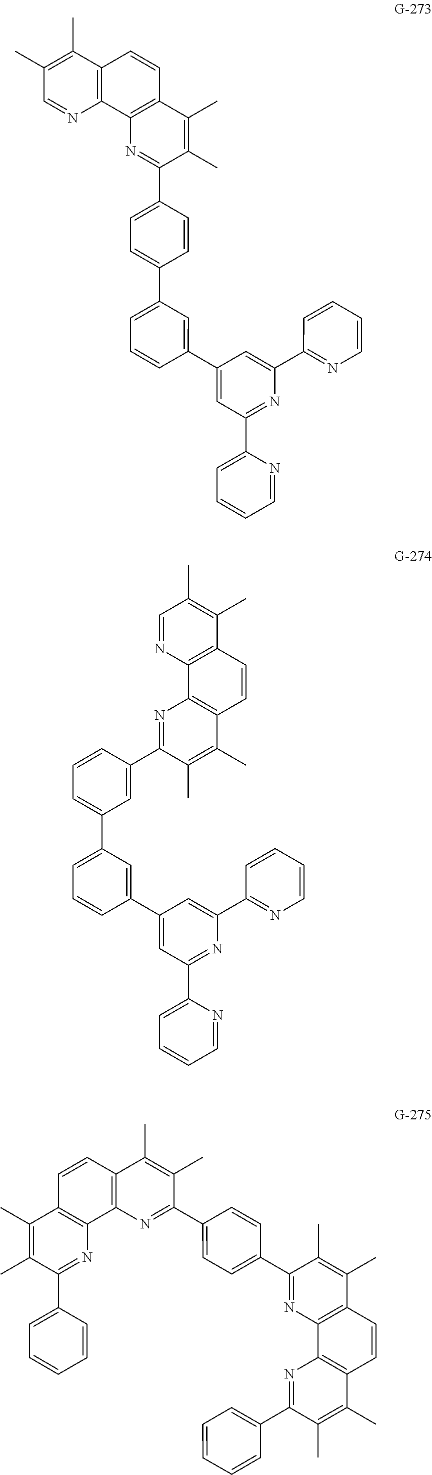 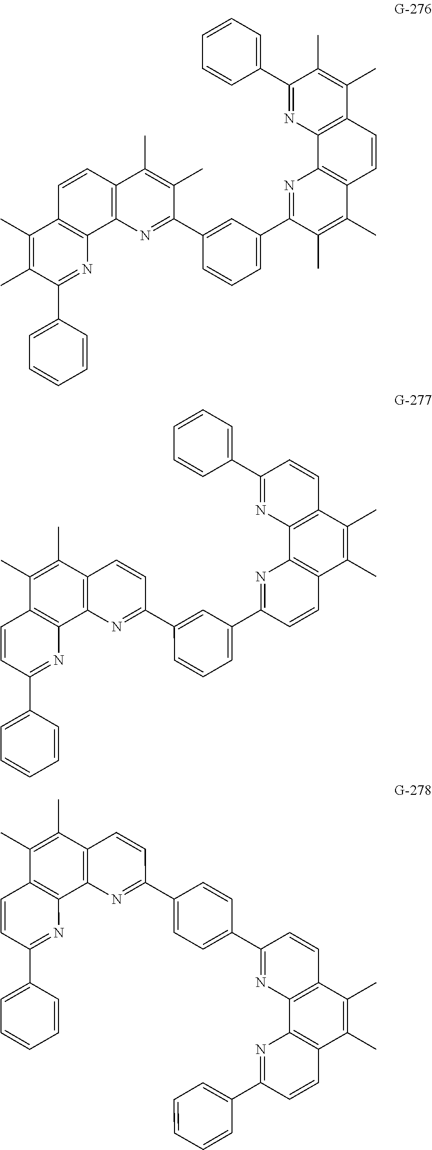 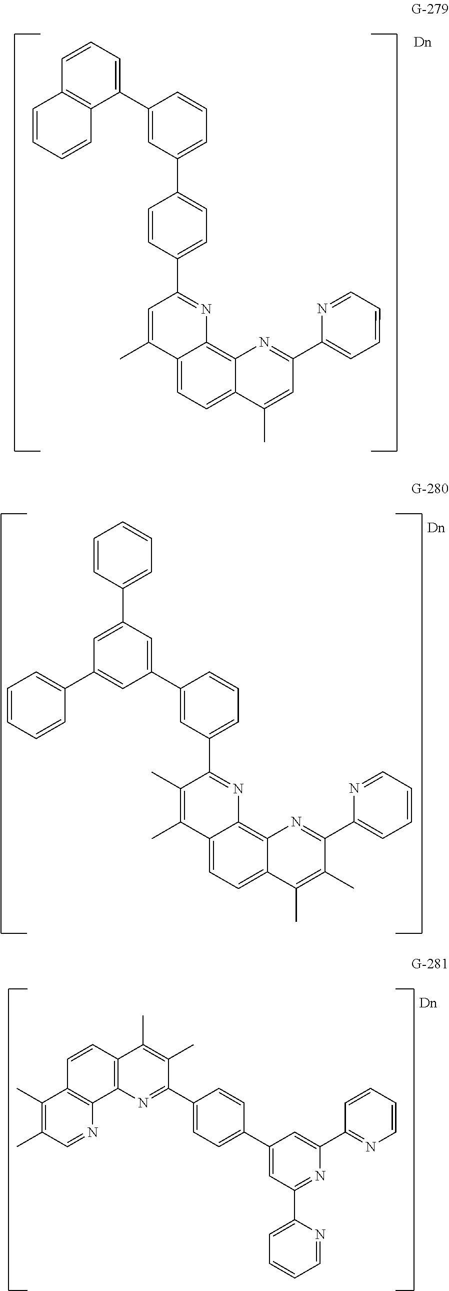 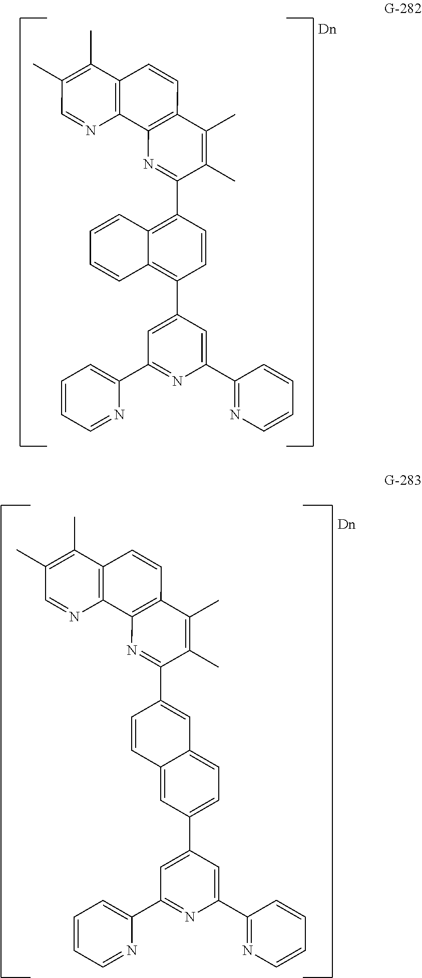 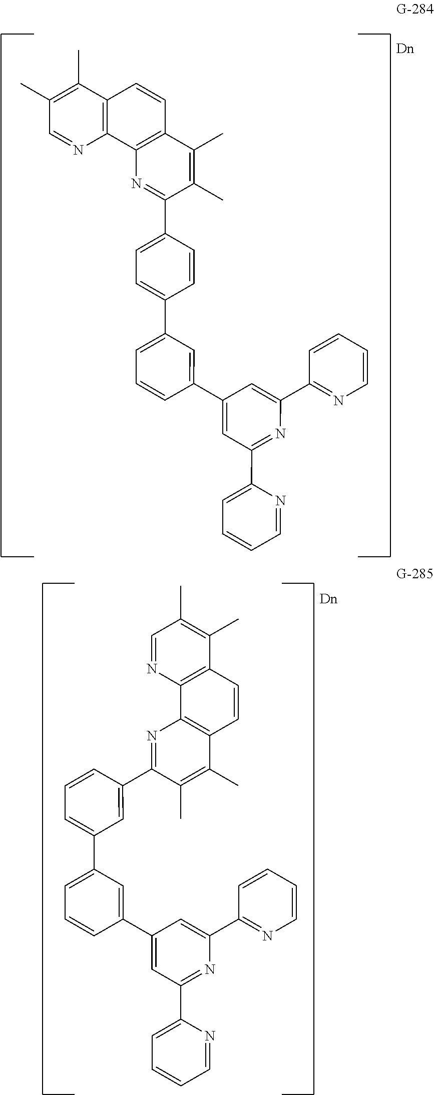 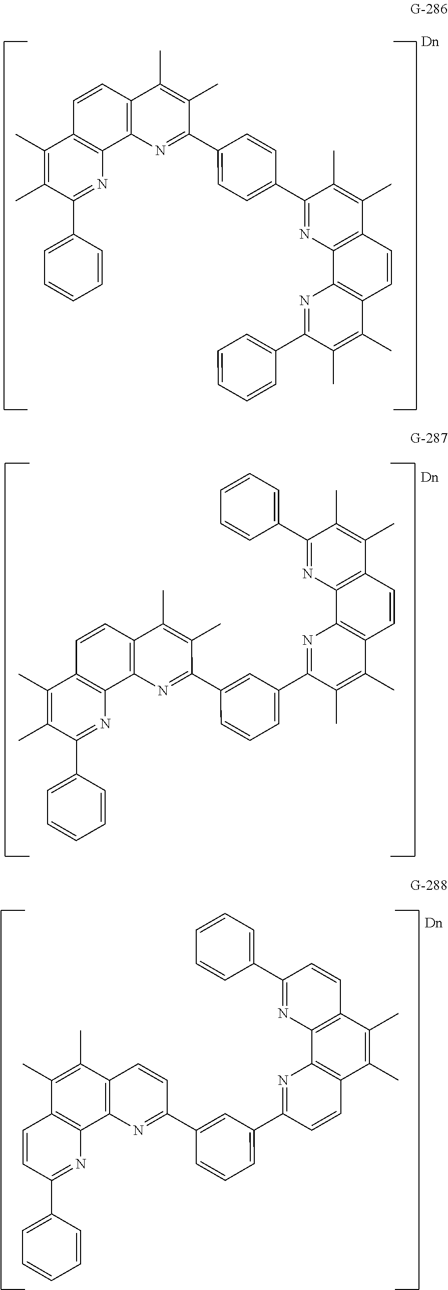 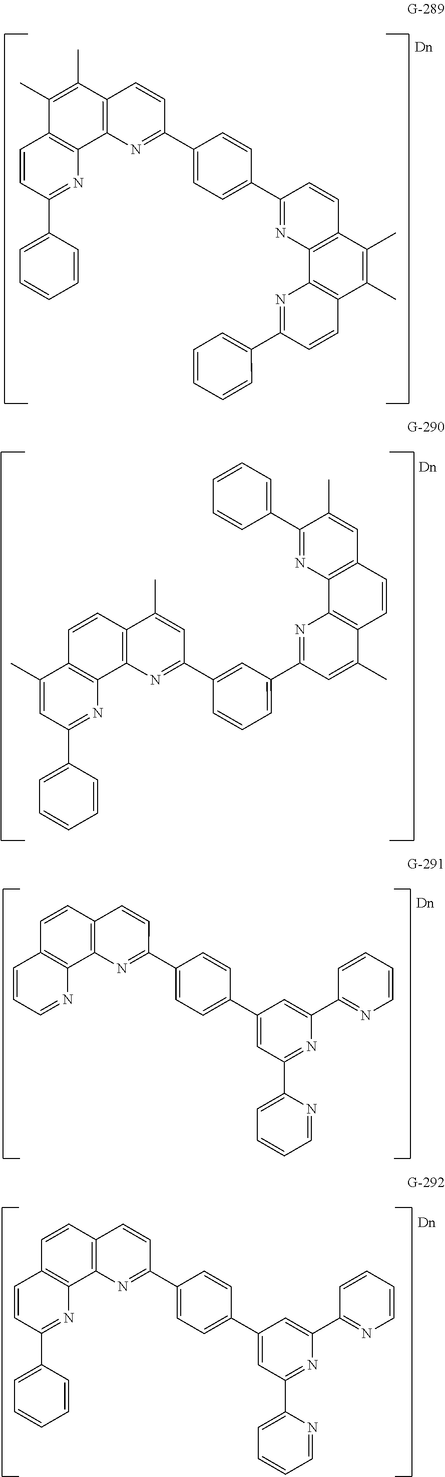 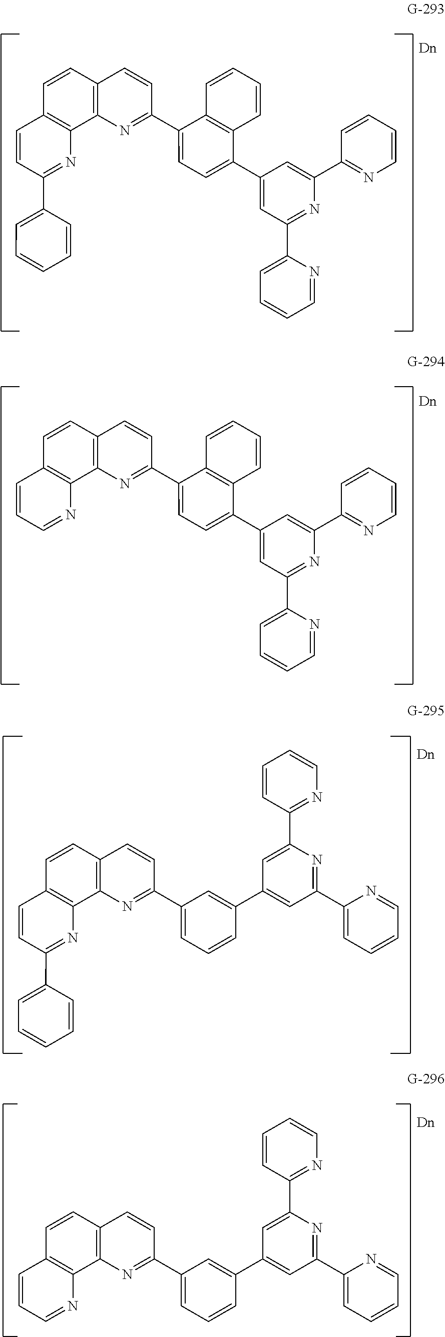 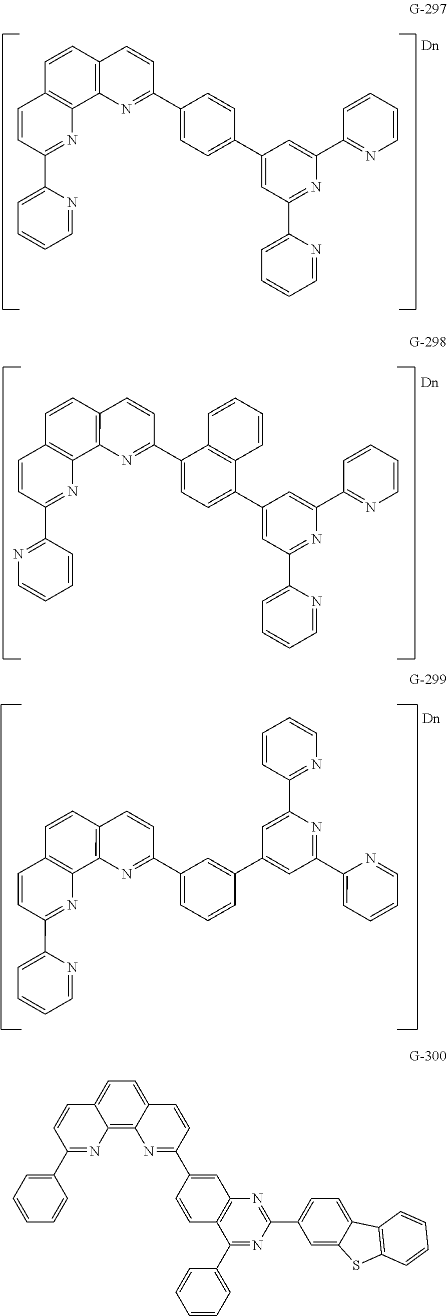 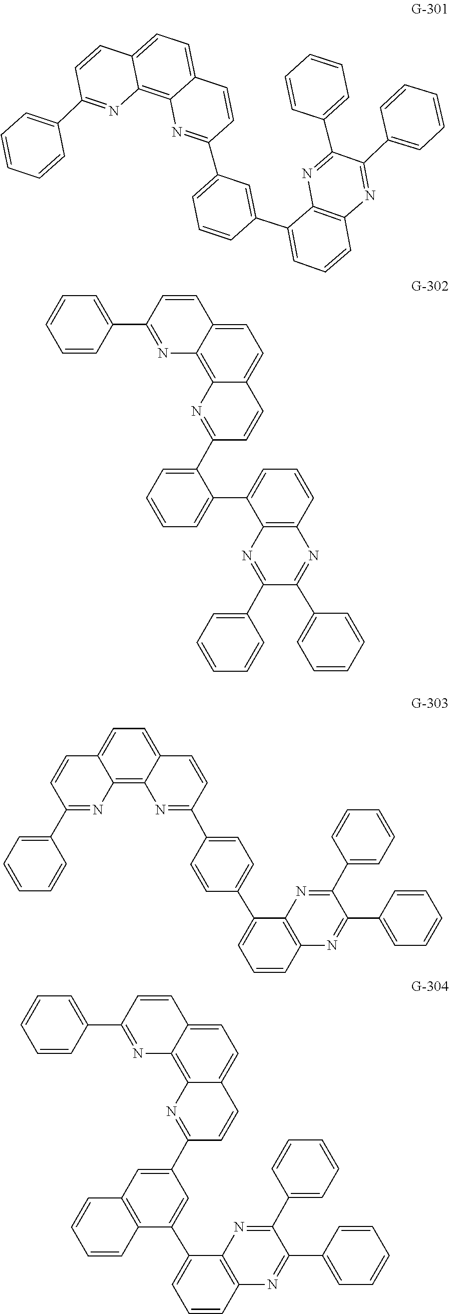 [SMILES: c1ccc(-c2ccc3ccc4ccc(-c5cccc(-c6cccc7nc(-c8ccccc8)c(-c8ccccc8)nc67)c5)nc4c3n2)cc1 ; c1ccc(-c2ccc3ccc4ccc(-c5ccccc5-c5cccc6nc(-c7ccccc7)c(-c7ccccc7)nc56)nc4c3n2)cc1 ; c1ccc(-c2ccc3ccc4ccc(-c5ccc(-c6cccc7nc(-c8ccccc8)c(-c8ccccc8)nc67)cc5)nc4c3n2)cc1 ; c1ccc(-c2ccc3ccc4ccc(-c5cc(-c6cccc7nc(-c8ccccc8)c(-c8ccccc8)nc67)c6ccccc6c5)nc4c3n2)cc1] 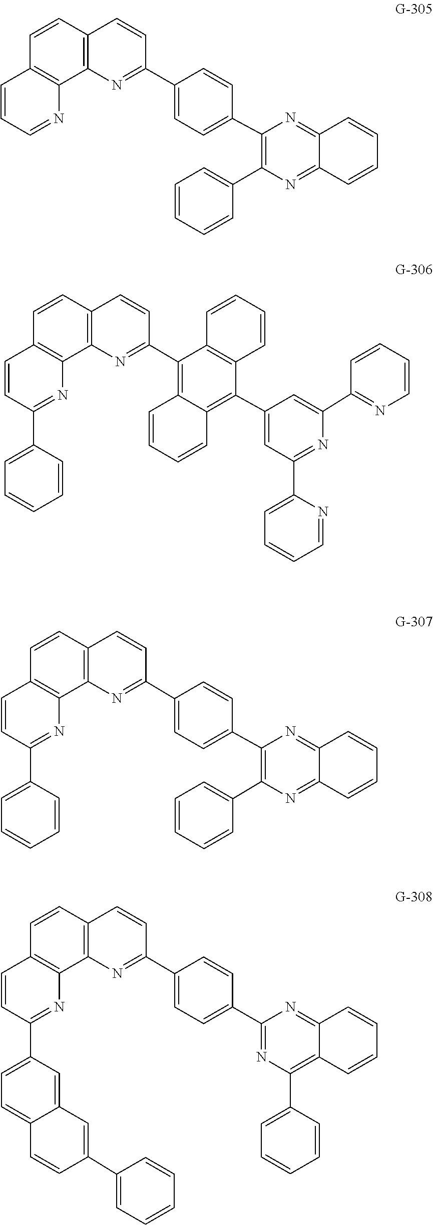 [SMILES: c1ccc(-c2nc3ccccc3nc2-c2ccc(-c3ccc4ccc5cccnc5c4n3)cc2)cc1 ; c1ccc(-c2ccc3ccc4ccc(-c5c6ccccc6c(-c6cc(-c7ccccn7)nc(-c7ccccn7)c6)c6ccccc56)nc4c3n2)cc1 ; c1ccc(-c2ccc3ccc4ccc(-c5ccc(-c6nc7ccccc7nc6-c6ccccc6)cc5)nc4c3n2)cc1 ; c1ccc(-c2ccc3ccc(-c4ccc5ccc6ccc(-c7ccc(-c8nc(-c9ccccc9)c9ccccc9n8)cc7)nc6c5n4)cc3c2)cc1] 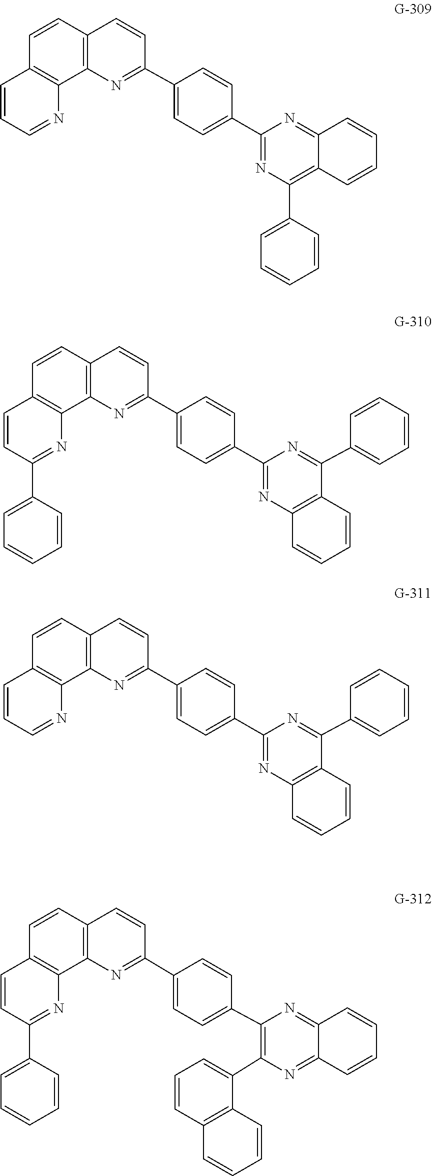 [SMILES: c1ccc(-c2nc(-c3ccc(-c4ccc5ccc6cccnc6c5n4)cc3)nc3ccccc23)cc1 ; c1ccc(-c2ccc3ccc4ccc(-c5ccc(-c6nc(-c7ccccc7)c7ccccc7n6)cc5)nc4c3n2)cc1 ; c1ccc(-c2nc(-c3ccc(-c4ccc5ccc6cccnc6c5n4)cc3)nc3ccccc23)cc1 ; c1ccc(-c2ccc3ccc4ccc(-c5ccc(-c6nc7ccccc7nc6-c6cccc7ccccc67)cc5)nc4c3n2)cc1] 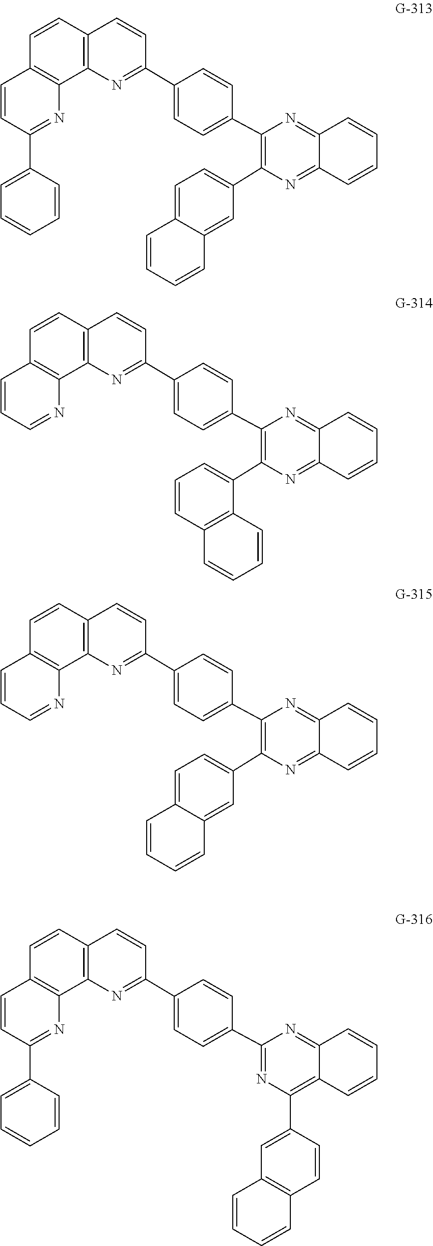 [SMILES: c1ccc(-c2ccc3ccc4ccc(-c5ccc(-c6nc7ccccc7nc6-c6ccc7ccccc7c6)cc5)nc4c3n2)cc1 ; c1ccc2c(-c3nc4ccccc4nc3-c3ccc(-c4ccc5ccc6cccnc6c5n4)cc3)cccc2c1 ; c1ccc2cc(-c3nc4ccccc4nc3-c3ccc(-c4ccc5ccc6cccnc6c5n4)cc3)ccc2c1 ; c1ccc(-c2ccc3ccc4ccc(-c5ccc(-c6nc(-c7ccc8ccccc8c7)c7ccccc7n6)cc5)nc4c3n2)cc1] 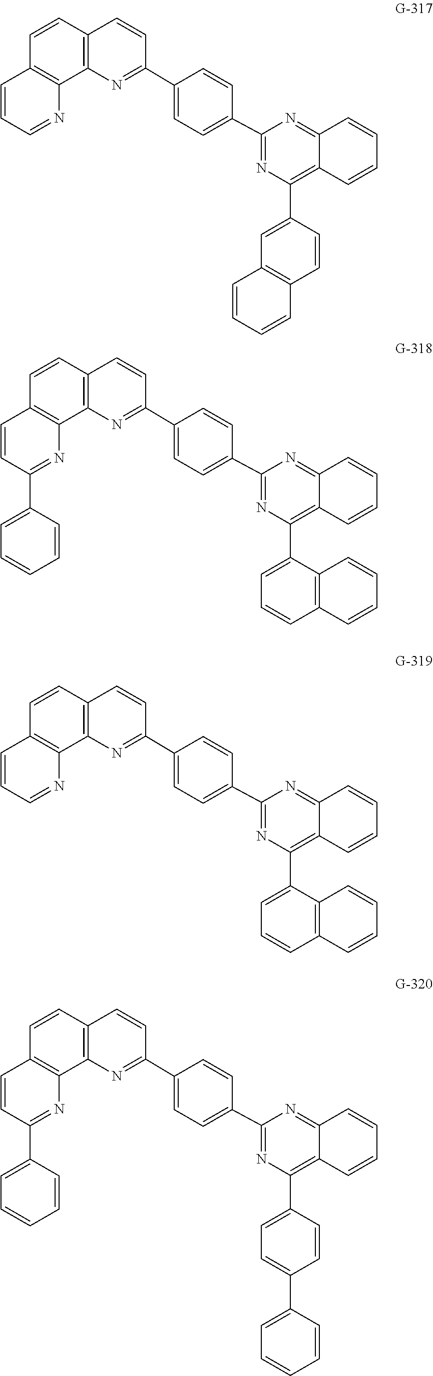 [SMILES: c1ccc2cc(-c3nc(-c4ccc(-c5ccc6ccc7cccnc7c6n5)cc4)nc4ccccc34)ccc2c1 ; c1ccc(-c2ccc3ccc4ccc(-c5ccc(-c6nc(-c7cccc8ccccc78)c7ccccc7n6)cc5)nc4c3n2)cc1 ; c1ccc2c(-c3nc(-c4ccc(-c5ccc6ccc7cccnc7c6n5)cc4)nc4ccccc34)cccc2c1 ; c1ccc(-c2ccc(-c3nc(-c4ccc(-c5ccc6ccc7ccc(-c8ccccc8)nc7c6n5)cc4)nc4ccccc34)cc2)cc1] 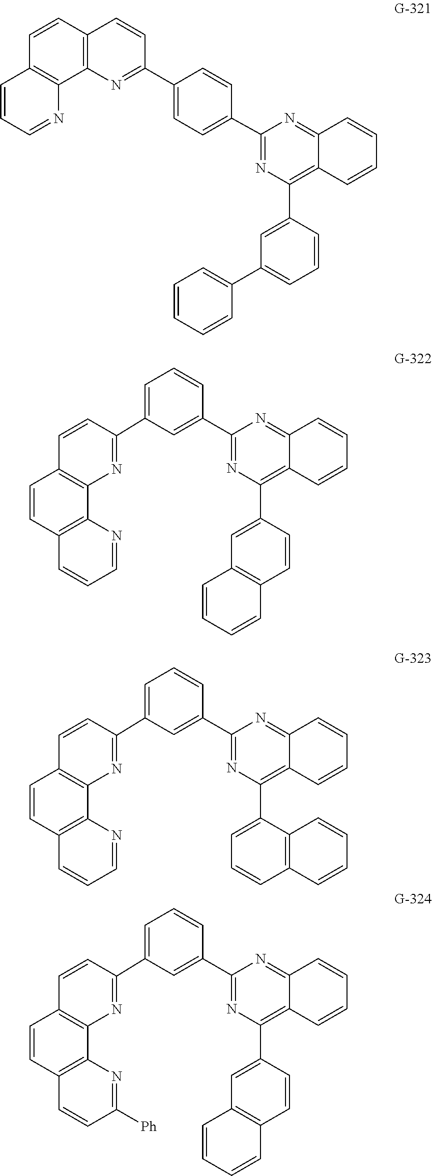 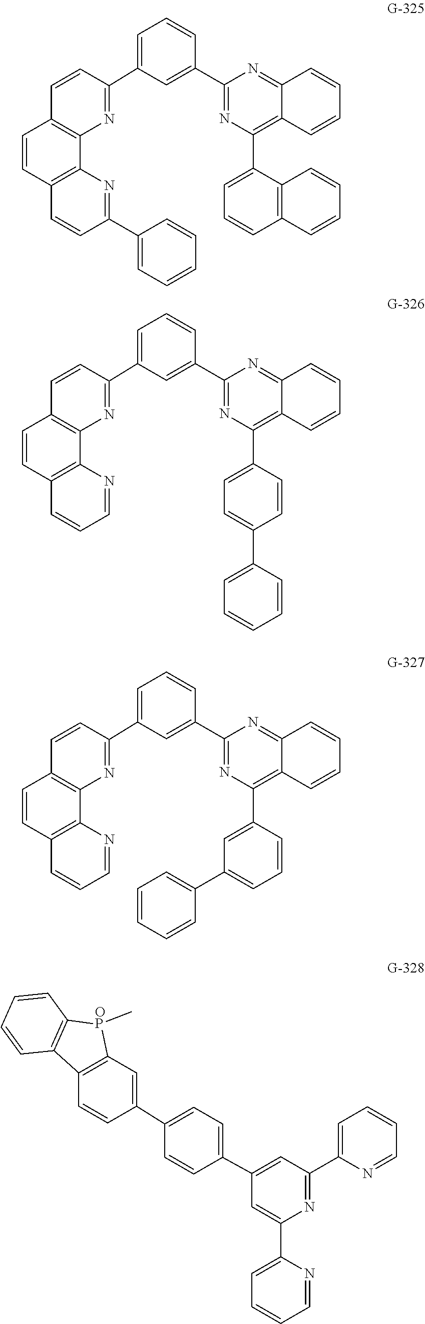 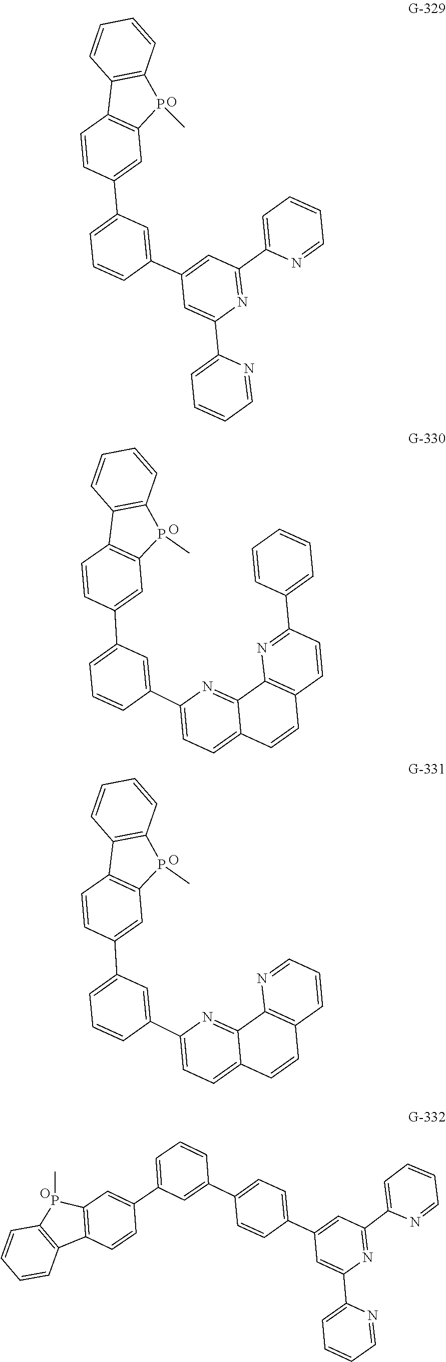 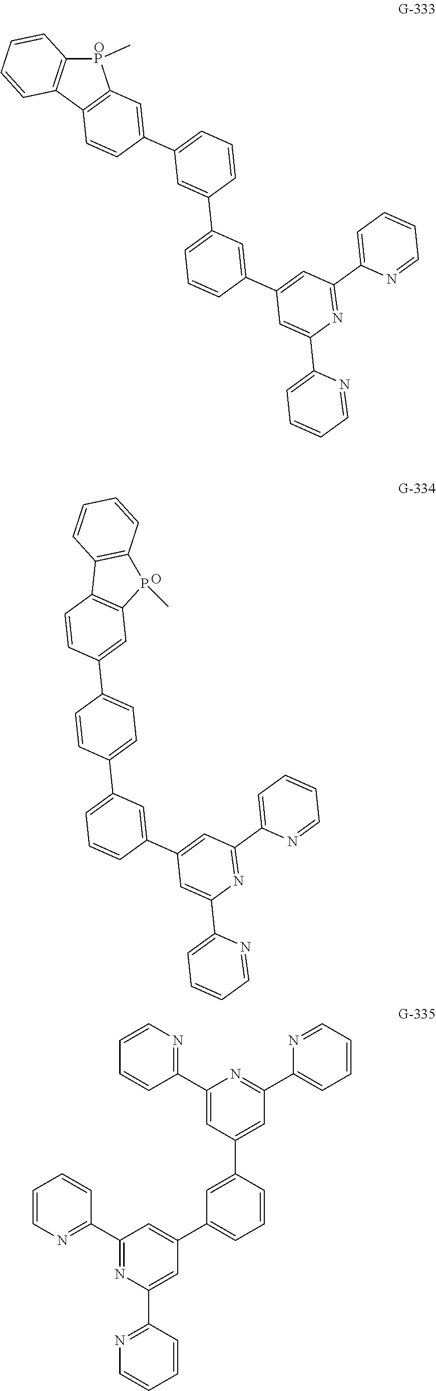 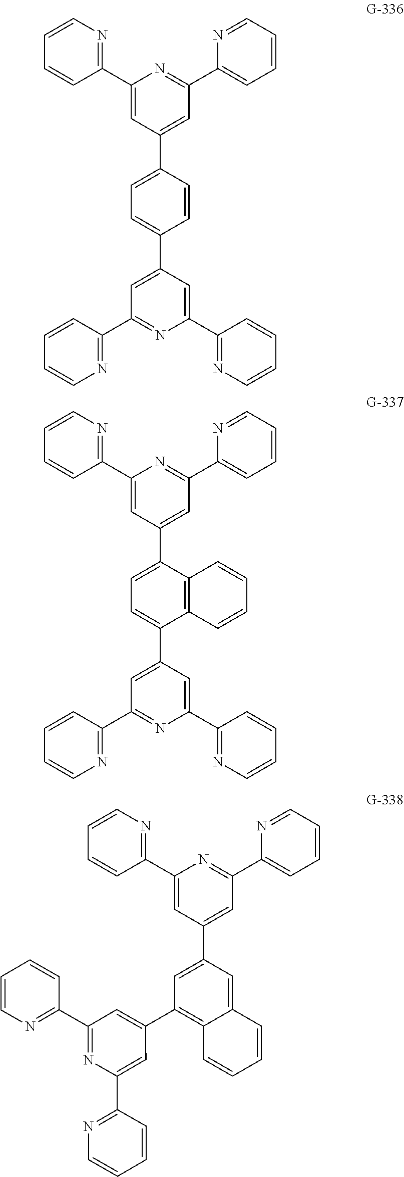 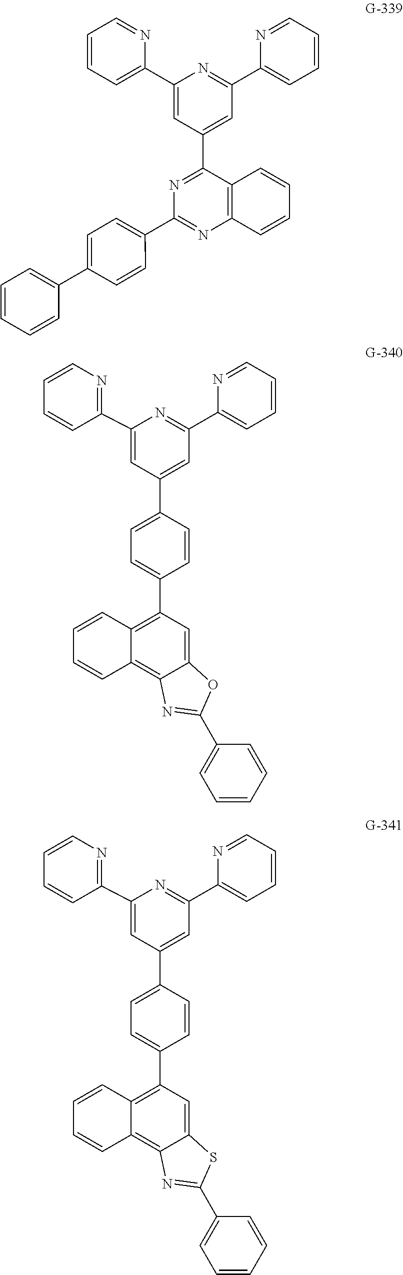 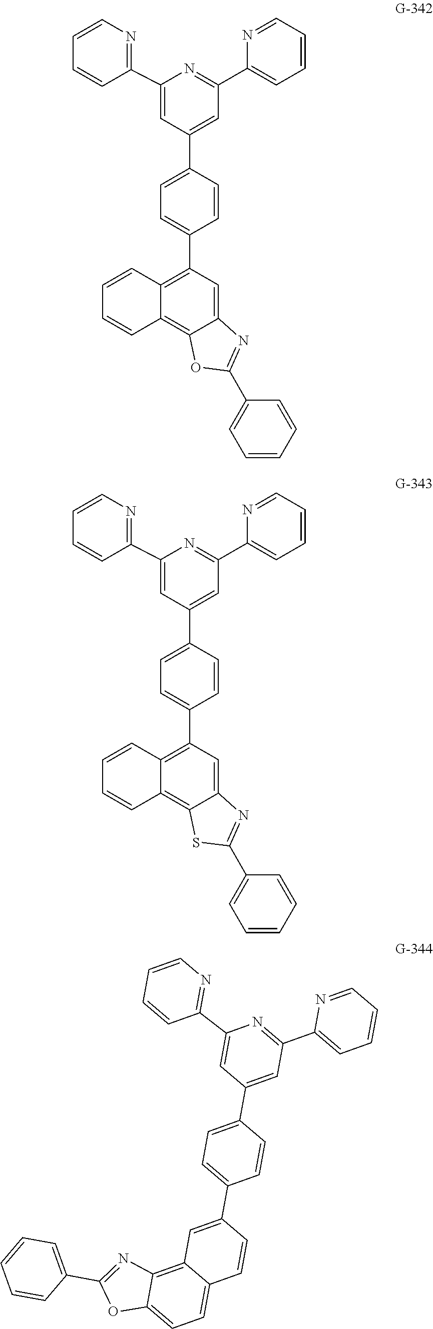 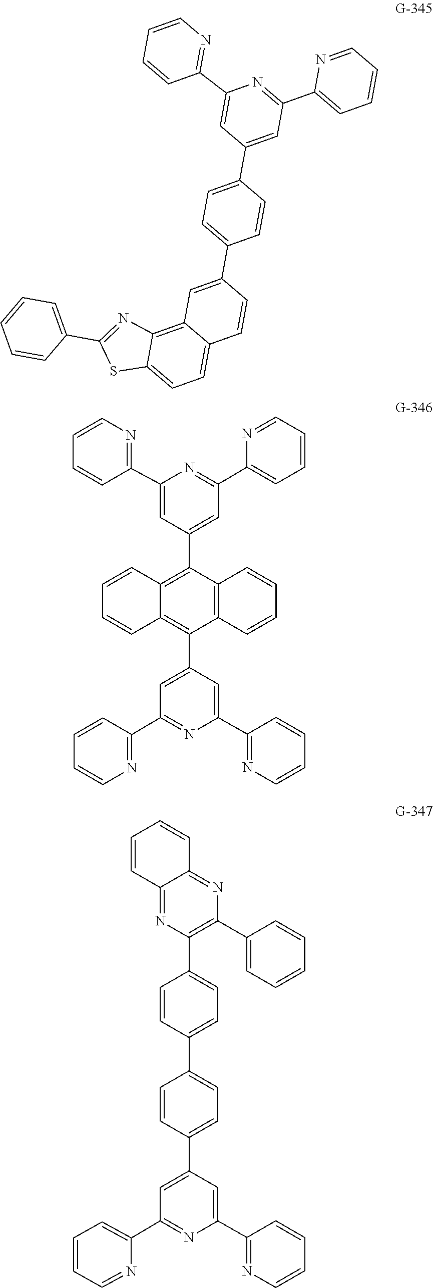 [SMILES: c1ccc(-c2nc3c(ccc4ccc(-c5ccc(-c6cc(-c7ccccn7)nc(-c7ccccn7)c6)cc5)cc43)s2)cc1 ; c1ccc(-c2cc(-c3c4ccccc4c(-c4cc(-c5ccccn5)nc(-c5ccccn5)c4)c4ccccc34)cc(-c3ccccn3)n2)nc1 ; c1ccc(-c2nc3ccccc3nc2-c2ccc(-c3ccc(-c4cc(-c5ccccn5)nc(-c5ccccn5)c4)cc3)cc2)cc1] 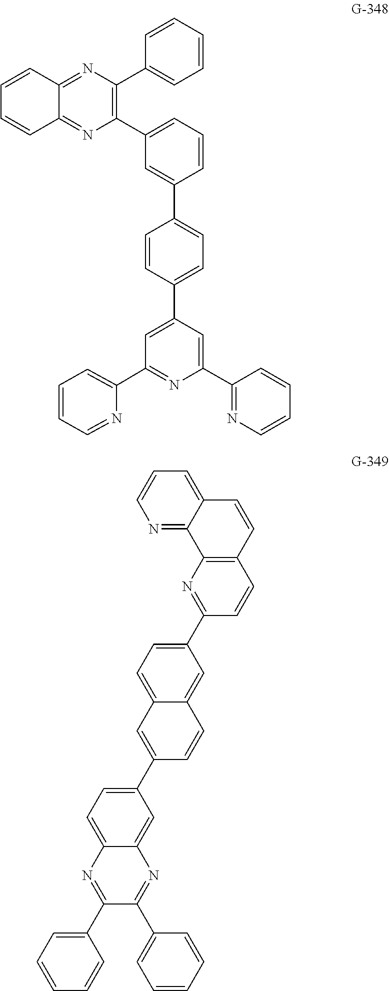 [SMILES: c1ccc(-c2nc3ccccc3nc2-c2cccc(-c3ccc(-c4cc(-c5ccccn5)nc(-c5ccccn5)c4)cc3)c2)cc1 ; c1ccc(-c2nc3ccc(-c4ccc5cc(-c6ccc7ccc8cccnc8c7n6)ccc5c4)cc3nc2-c2ccccc2)cc1] 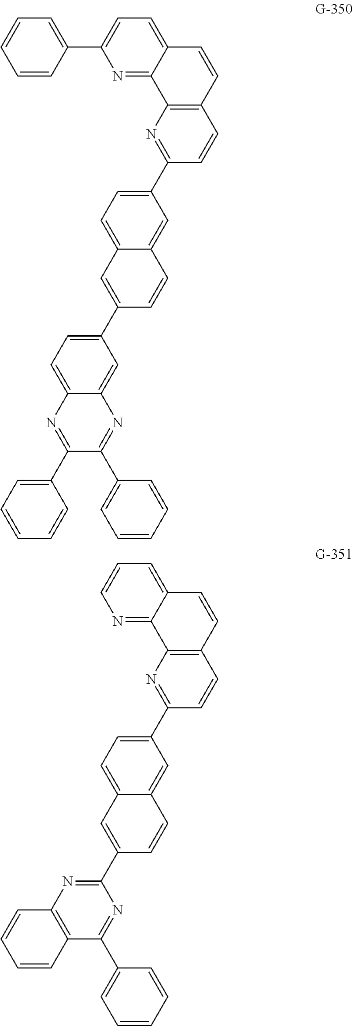 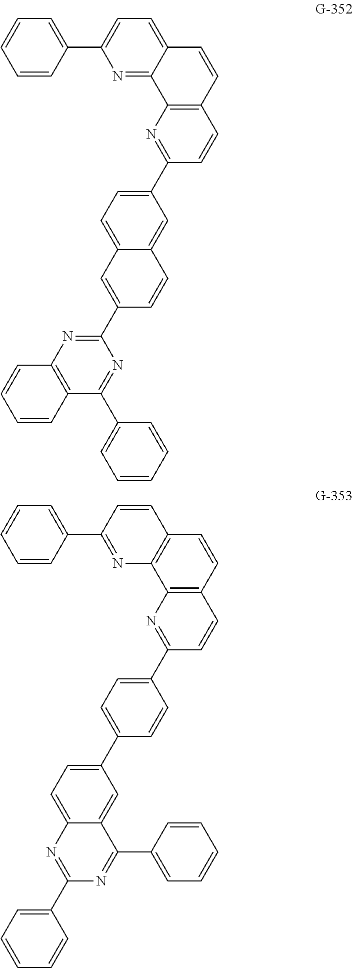 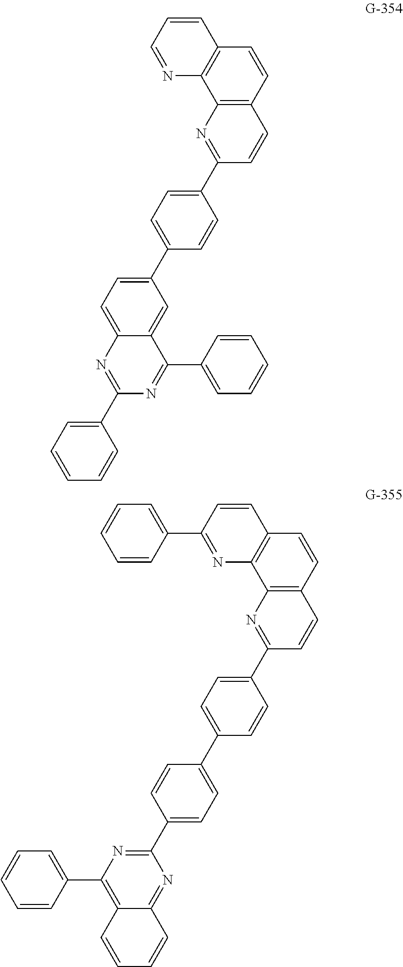 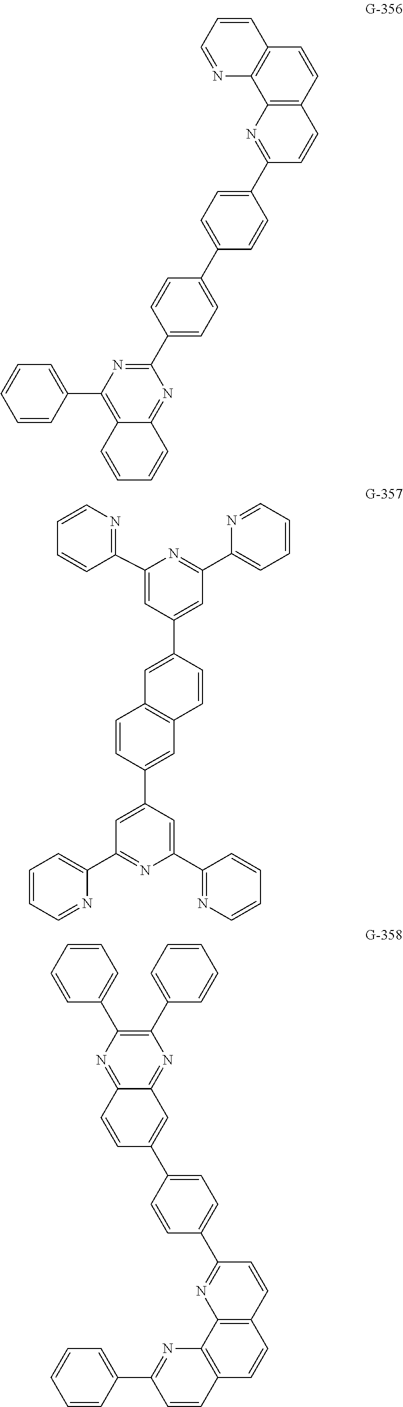 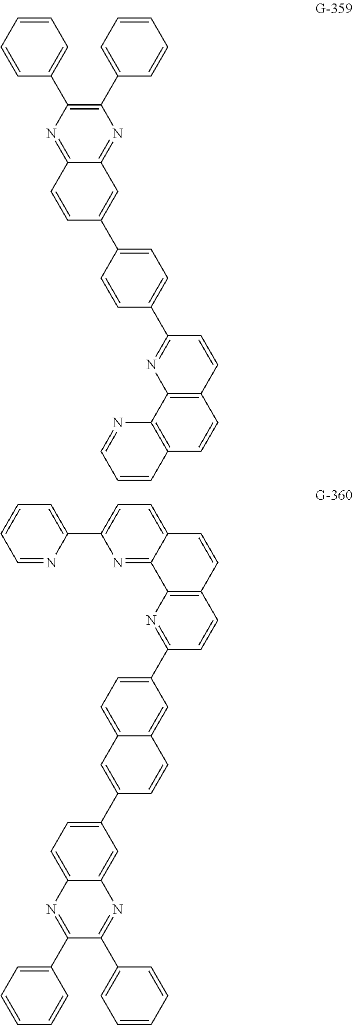 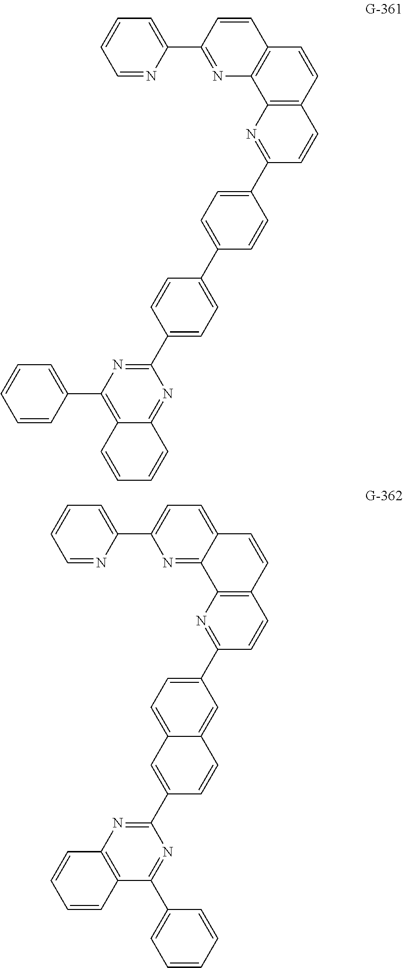 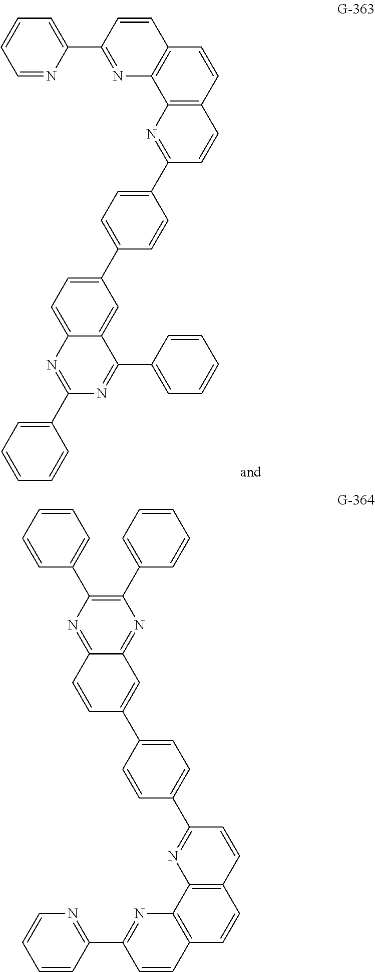
In the compounds, Dₙ indicates that n hydrogen atoms are replaced with deuterium atoms, wherein n is an integer ranging from 1 to the maximum number of hydrogen atoms present in the compound.

22. An organic electroluminescent device comprising:
a first electrode;
a second electrode opposing the first electrode; and
two or more light-emitting units disposed between the first electrode and the second electrode, and including at least one light-emitting layer,
wherein at least one of the light-emitting units sequentially comprises a first light-emitting layer, a second light-emitting layer, and a third light-emitting layer, which are adjacent to one another,
wherein the first light-emitting layer is positioned close to the first electrode, and the third light-emitting layer is positioned close to the second electrode, and
wherein the first light-emitting layer and the third light-emitting layer comprise the same compound.

23. An organic electroluminescent device comprising:
a first electrode;
a second electrode opposing the first electrode; and
a first light-emitting layer, a second light-emitting layer, and a third light-emitting layer which are adjacent to one another, sequentially, between the first electrode and the second electrode,
wherein the first light-emitting layer is positioned close to the first electrode, and the third light-emitting layer is positioned close to the second electrode, and
wherein each of the host materials of the two adjacent light-emitting layers is different from each other.

24. The organic electroluminescent device according to claim 23, wherein the host materials of each of the first light-emitting layer, the second light-emitting layer, and the third light-emitting layer comprise different compounds.

25. The organic electroluminescent device according to claim 23, wherein the host materials of each of the first light-emitting layer and the third light-emitting layer comprise the same compound.

26. The organic electroluminescent device according to claim 23, wherein at least two layers of the host materials of each of the first light-emitting layer, the second light-emitting layer, and the third light-emitting layer comprise a composition including two or more compounds, wherein the composition of each layer comprises the same compound but the content ratios of the compounds are different.
